(19) United States
(12) Patent Application Publication
(10) Pub. No.: US 2009/0215046 A1
(43) Pub. Date: Aug. 27, 2009

(54) Novel nucleotide and amino acid sequences, and assays methods of use thereof for diagnosis of colon cancer

(75) Inventors: Rotem Sorek, Rechovot (IL); Michal Ayalon-Soffer, Ramat-HaSharon (IL); Dvir Dahary, Tel-Aviv (IL); Amit Novik, Beit-HaSharon (IL); Amir Toporik, Azur (IL); Alexander Diber, Richon-LeZion (IL); Guy Kol, Givat Shmuel (IL); Sarah Pollock, Tel-Aviv (IL); Zurit Levine, Herzlia (IL); Gad S. Cojocaru, Ramat-HaSharon (IL); Ronen Shemesh, Modiln (IL); Yossi Cohen, Banstead (GB); Osnat Sella-Tavor, Kfar Kish (IL); Shirley Sameah-Greenwald, Kfar-Saba (IL); Shira Walach, Hod-HaSharon (IL)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(73) Assignee: COMPUGEN LTD., Tel-Aviv (IL)

(21) Appl. No.: 11/976,324

(22) Filed: Oct. 23, 2007

Related U.S. Application Data
(63) Continuation-in-part of application No. 11/050,875, filed on Jan. 27, 2005, now abandoned.
(60) Provisional application No. 60/620,916, filed on Oct. 22, 2004.
(60) Provisional application No. 60/628,123, filed on Nov. 17, 2004.
(60) Provisional application No. 60/621,131, filed on Oct. 25, 2004.
(60) Provisional application No. 60/628,145, filed on Nov. 17, 2004.
(60) Provisional application No. 60/620,656, filed on Oct. 22, 2004.
(60) Provisional application No. 60/620,975, filed on Oct. 22, 2004.
(60) Provisional application No. 60/539,129, filed on Jan. 27, 2004.

(51) Int. Cl. C12Q 1/68; C07H 21/02; C07K 14/47; C12N 15/63; C12Q 1/02; C07K 17/00; G01N 33/574; C12N 5/06; C12P 21/00

(52) U.S. Cl. 435/6; 536/23.5; 530/300; 435/320.1; 435/325; 435/69.1; 435/7.23; 435/7.92; 435/29; 435/7.1; 530/402

(57) ABSTRACT

Novel markers for colon cancer that are both sensitive and accurate. These markers are overexpressed in colon cancer specifically, as opposed to normal colon tissue. The measurement of these markers, alone or in combination, in patient samples provides information that the diagnostician can correlate with a probable diagnosis of colon cancer. The markers of the present invention, alone or in combination, show a high degree of differential detection between colon cancer and non-cancerous states.

### CROSS-REFERENCE TO RELATED APPLICATION(S)

This application is a CIP application of application Ser. No. 11/050,875 filed Jan. 27 2005.

This application is related to Novel Nucleotide and Amino Acid Sequences, and Assays and Methods of use thereof for Diagnosis of Colon Cancer, and claims priority to the below U.S. provisional applications which are incorporated by reference herein:

Application No. 60/620,916 filed Oct. 22, 2004—Differential Expression of Markers in Colon Cancer

Application No. 60/628,123 filed Nov. 17, 2004—Differential Expression of Markers in Colon Cancer II

Application No. 60/621,131 filed Oct. 25, 2004—Diagnostic Markers for Colon Cancer, and Assays and Methods of use thereof.

Application No. 60/628,145 filed Nov. 17, 2004—Differential Expression of Markers in Pancreatic Cancer II

Application No. 60/620,656 filed Oct. 22, 2004—Differential Expression of Markers in Prostate Cancer

Application No. 60/620,975 filed Oct. 22, 2004—Differential Expression of Markers in Brain Cancer

Application No. 60/539,129 filed Jan. 27, 2004—Methods and Systems for Annotating Biomolecular Sequences.

### FIELD OF THE INVENTION

The present invention is related to novel nucleotide and protein sequences that are diagnostic markers for colon cancer, and assays and methods of use thereof.

### BACKGROUND OF THE INVENTION

Colon and rectal cancers are malignant conditions which occur in the corresponding segments of the large intestine. These cancers are sometimes referred to jointly as “colorectal cancer”, and, in many respects, the diseases are considered identical. The major differences between them are the sites where the malignant growths occur and the fact that treatments may differ based on the location of the tumors.

More than 95 percent of cancers of the colon and rectum are adenocarcinomas, which develop in glandular cells lining the inside (lumen) of the colon and rectum. In addition to adenocarcinomas, there are other rarer types of cancers of the large intestine: these include carcinoid tumors usually found in the appendix and rectum; gastrointestinal stromal tumors found in connective tissue in the wall of the colon and rectum; and lymphomas, which are malignancies of immune cells in the colon, rectum and lymph nodes. As with other malignant conditions, a number of genetic abnormalities have been associated with colon tumors (Bos et al, (1987) Nature 327:293-297; Baker et al, (1989) 244:217-2221; Nishisho et al, (1991) 253:665-669).

Colorectal cancer is the second most common cause of cancer death in the United States and the third most prevalent cancer in both men and women. Approximately 100,000 patients every year suffer from colon cancer and approximately half that number die of the disease. In large part this death rate is due to the inability to diagnose the disease at an early stage (Wanebo (1993) Colorectal Cancer, Mosby, St. Louis Mo.). In fact, the prognosis for a case of colon cancer is vastly enhanced when malignant tissue is detected at the early stage known as polyps. Polyps are usually benign growths protruding from the mucous membrane. Nearly all cases of colorectal cancer arise from adenomatous polyps, some of which mature into large polyps, undergo abnormal growth and development, and ultimately progress into cancer. This progression would appear to take at least 10 years in most patients, rendering it a readily treatable form of cancer if diagnosed early, when the cancer is localized. Simple removal of malignant polyps (polypectomy) through colonoscopy is now routine, and curing the condition from this procedure is effectively guaranteed. However, early detection of polyps and tumors depends on diligent and ongoing examination of patients at risk. The most reliable detection procedures to date include fecal occult blood tests, sigmoidoscopy, barium enema X-ray, digital rectal exam, and colonoscopy. Normally a malignant colon cancer will not cause noticeable symptoms (e.g., bowel obstruction, abdominal pain, anemia) until it has reached an advanced and far more serious stage of malignancy. At these stages, only risky, traumatic and/or invasive procedures are available, including chemotherapy, radiation therapy, and colonectomy.

Although current understanding of the etiology of colon cancer is undergoing continual refinement, extensive research in this area points to a combination of factors, including age, hereditary and non-hereditary conditions, and environmental/dietary factors. Age is a key risk factor in the development of colorectal cancer, since men and women over 40 years of age become increasingly susceptible to that cancer. Incidence rates increase considerably in each subsequent decade of life. A number of hereditary and nonhereditary conditions have also been linked to a heightened risk of developing colorectal cancer, including familial adenomatous polyposis (FAP), hereditary nonpolyposis colorectal cancer (Lynch syndrome or HNPCC), a personal and/or family history of colorectal cancer or adenomatous polyps, inflammatory bowel disease, diabetes mellitus, and obesity.

In the case of FAP, the tumor suppressor gene APC (adenomatous polyposis coli), located at 5q21, has been either mutationally inactivated or deleted (Alberts et al., Molecular Biology of the Cell 1288 (3d ed. 1994)). The APC protein plays a role in a number of functions, including cell adhesion, apoptosis, and repression of the c-myc oncogene. Of those patients with colorectal cancer who have normal APC genes, over 65% have such mutations in the cancer cells but not in other tissues. In the case of HPNCC, patients manifest abnormalities in the tumor suppressor gene HNPCC, but only about 15% of tumors contain the mutated gene. A host of other genes have also been implicated in colorectal cancer, including the K-ras, c-Ki-ras, N-ras, H-ras and c-myc oncogenes, and the tumor suppressor genes DCC (deleted in colon carcinoma), Wg/Wnt signal transduction pathway components and p53.

Some tyrosine kinases have been shown up-regulated in colorectal tumor tissues or cell lines like HT29. Focal adhesion kinase (FAK) and its up-stream kinase c-src and c-yes in colonic epithelial cells may play an important role in the promotion of colorectal cancers through the extracellular 1 5 matrix (ECM) and integrin-mediated signaling pathways. The formation of c-src/FAK complexes may coordinately deregulate VEGF expression and apoptosis inhibition.

Recent evidences suggest that a specific signal-transduction pathway for cell survival that implicates integrin engagement leads to FAK activation and thus activates PI-3 kinase and akt. In turn, akt phosphorylates BAD and blocks apoptosis in epithelial cells. The activation of c-src in colon cancer may induce VEGF expression through the hypoxia pathway. Other genes that may be implicated in colorectal cancer include Cox enzymes (Ota, S. et al. Aliment Pharmacol. Ther. 16 (Suppl 2): 102-106 (2002)), estrogen (alAzzawi, F. and Wahab, M. Climacteric 5: 3-14 (2002)), peroxisome proliferator-activated receptor-y (PPAR-y) (Gelman, L. et al. Cell Mol. Life. Sci. 5 5: 932-943 (1999)), IGF-I (Giovannucci (2001)), thymine DNA glycosylase (TDG) (Hardeland, U. et al. Prog. Nucleic Acid Res. Mol. Biol. 68: 235-253 (2001)) and EGF (Mendelsohn, J. EndocrineRelated Cancer 8: 3-9 (2001)).

Procedures used for detecting, diagnosing, monitoring, staging, and prognosticating colon cancer are of critical importance to the outcome of the patient. For example, patients diagnosed with early colon cancer generally have a much greater five-year survival rate as compared to the survival rate for patients diagnosed with distant metastasized colon cancer. Because colon cancer is highly treatable when detected at an early, localized stage, screening should be a part of routine care for all adults starting at age 50, especially those with first-degree relatives with colorectal cancer. One major advantage of colorectal cancer screening over its counterparts in other types of cancer is its ability to not only detect precancerous lesions, but to remove them as well. The key colorectal cancer screening tests in use today are fecal occult blood test, sigmoidoscopy, colonoscopy, double-contrast barium enema, and the carcinoembryonic antigen (CEA) test. New diagnostic methods which are more sensitive and specific for detecting early colon cancer are clearly needed.

Visual examination of the colon for abnormalities can be performed through endoscopic or radiographic techniques such as rigid proctosigmoidoscopy, flexible sigmoidoscopy, colonoscopy, and barium-contrast enema. These methods enable one to detect, biopsy, and remove adenomatous polyps. Despite the advantages of these procedures, there are accompanying downsides: they are expensive, and uncomfortable, and also carry with them a risk of complications. Sigmoidoscopy, by definition, is limited to the sigmoid colon and below, colonoscopy is a relatively expensive procedure, and both share the risk of possible bowel perforation and hemorrhaging. Double-contrast barium enema (DCBE) enables detection of lesions better than FOBT, and almost as well a colonoscopy, but it may be limited in evaluating the winding rectosigmoid region.

Another method of colon cancer diagnosis is the detection of carcinoembryonic antigen (CEA) in a blood sample from a subject, which when present at high levels, may indicate the presence of advanced colon cancer. But CEA levels may also be abnormally high when no cancer is present. Thus, this test is not selective for colon cancer, which limits the test's value as an accurate and reliable diagnostic tool. In addition, elevated CEA levels are not detectable until late-stage colon cancer, when the cure rate is low, treatment options limited, and patient prognosis poor.

Several classification systems have been devised to stage the extent of colorectal cancer, including the Dukes' system and the more detailed International Union against Cancer-American Joint Committee on Cancer TNM staging system, which is considered by many in the field to be a more useful staging system. These most widely used staging systems generally use at least one of the following characteristics for staging: the extent of tumor penetration into the colon wall, with greater penetration generally correlating with a more dangerous tumor; the extent of invasion of the tumor through the colon wall and into other neighboring tissues, with greater invasion generally correlating with a more dangerous tumor; the extent of invasion of the tumor into the regional lymph nodes, with greater invasion generally correlating with a more dangerous tumor; and the extent of metastatic invasion into more distant tissues, such as the liver, with greater metastatic invasion generally correlating with a more dangerous disease state.

“Dukes A” and “Dukes B” colon cancers are neoplasia that have invaded into the wall of the colon but have not spread into other tissues. Dukes A colon cancers are cancers that have not invaded beyond the submucosa. Dukes B colon cancers are subdivided into two groups: Dukes B1 and Dukes B2. “Dukes B1” colon cancers are neoplasias that have invaded up to but not through the muscularis propria. Dukes B2 colon cancers are cancers that have breached completely through the muscularis propria. Over a five year period, patients with Dukes A cancer who receive surgical treatment (i.e. removal of the affected tissue) have a greater than 90% survival rate. Over the same period, patients with Dukes B1 and Dukes B2 cancer receiving surgical treatment have a survival rate of about 85% and 75% respectively. Dukes A, B1 and B2 cancers are also referred to as T1, T2 and T3-T4 cancers, respectively. “Dukes C” colon cancers are cancers that have spread to the regional lymph nodes, such as the lymph nodes of the gut. Patients with Dukes C cancer who receive surgical treatment alone have a 35% survival rate over a five year period, but this survival rate is increased to 60% in patients that receive chemotherapy. “Dukes D” colon cancers are cancers that have metastasized to other organs. The liver is the most common organ in which metastatic colon cancer is found. Patients with Dukes D colon cancer have a survival rate of less than 5% over a five year period, regardless of the treatment regimen.

The TNM system, which is used for either clinical or pathological staging, is divided into four stages, each of which evaluates the extent of cancer growth with respect to primary tumor (T), regional lymph nodes (N), and distant metastasis (M). The system focuses on the extent of tumor invasion into the intestinal wall, invasion of adjacent structures, the number of regional lymph nodes that have been affected, and whether distant metastasis has occurred. Stage 0 is characterized by in situ carcinoma (Tis), in which the cancer cells are located inside the glandular basement membrane (intraepithelial) or lamina propria, (intramucosal). In this stage, the cancer has not spread to the regional lymph nodes (NO), and there is no distant metastasis (N40). In stage 1, there is still no spread of the cancer to the regional lymph nodes and no distant metastasis, but the tumor has invaded the submucosa (T1) or has progressed further to invade the muscularis propria (T2). Stage R also involves no spread of the cancer to the regional lymph nodes and no distant metastasis, but the tumor has invaded the subserosa, or the nonperitonealized pericolic or perirectal tissues (T3), or has progressed to invade other organs or structures, and/or has perforated the visceral peritoneum (T4). Stage 3 is characterized by any of the T substages, no distant metastasis, and either metastasis in 1 to 3 regional lymph nodes (N1) or metastasis in four or more regional lymph nodes (N2). Lastly, stage 4 involves any of the T or N substages, as well as distant metastasis.

Currently, pathological staging of colon cancer is preferable over clinical staging as pathological staging provides a more accurate prognosis. Pathological staging typically involves examination of the resected colon section, along with surgical examination of the abdominal cavity.

### SUMMARY OF THE INVENTION

The background art does not teach or suggest markers for colon cancer that are sufficiently sensitive and/or accurate, alone or in combination. From the foregoing, it is clear that procedures used for detecting, diagnosing, monitoring, staging, prognosticating, and preventing the recurrence of colorectal cancer are of critical importance to the outcome of the patient. Moreover, current procedures, while helpful in each of these analyses, are limited by their specificity, sensitivity, invasiveness, and/or their cost. It would therefore be desirable to provide more sensitive and accurate methods and reagents for the early diagnosis, staging, prognosis, monitoring, and treatment of diseases associated with colon cancer, or to indicate a predisposition to such for preventative measures, as well as to determine whether or not such cancer has metastasized and for monitoring the progress of colon cancer in a human which has not metastasized for the onset of metastasis.

The present invention overcomes the deficiencies of the background art by providing novel markers for colon cancer that are both sensitive and accurate. Furthermore, these markers are able to distinguish between different stages of colon cancer, such as adenocarcinoma (mucinous or signet ring cell originating); leiomyocarcomas; carcinoid. Furthermore, at least some of these markers are able to distinguish, alone or in combination, between colon cancer between non-cancerous polyps. These markers are overexpressed in colon cancer specifically, as opposed to normal colon tissue. The measurement of these markers, alone or in combination, in patient samples provides information that the diagnostician can correlate with a probable diagnosis of colon cancer. The markers of the present invention, alone or in combination, show a high degree of differential detection between colon cancer and non-cancerous states.

According to preferred embodiments of the present invention, examples of suitable biological samples include but are not limited to blood, serum, plasma, blood cells, urine, sputum, saliva, stool, spinal fluid or CSF, lymph fluid, the external secretions of the skin, respiratory, intestinal, and genitourinary tracts, tears, milk, neuronal tissue, colon tissue or mucous and any human organ or tissue. In a preferred embodiment, the biological sample comprises colon tissue and/or a serum sample and/or a urine sample and/or a stool sample and/or any other tissue or liquid sample. The sample can optionally be diluted with a suitable eluant before contacting the sample to an antibody and/or performing any other diagnostic assay.

Information given in the text with regard to cellular localization was determined according to four different software programs: (i) tmhmm (from Center for Biological Sequence Analysis, Technical University of Denmark DTU, http://www.cbs.dtu.dk/services/TMHMM/TMHMM2.0b.guide.php) or (ii) tmpred (from EMBnet, maintained by the ISREC Bionformatics group and the LICR Information Technology Office, Ludwig Institute for Cancer Research, Swiss Institute of Bioinformatics, http://www.ch.embnet.org/software/TMPRED_form.html) for transmembrane region prediction; (iii) signalp_hmm or (iv) signalp_nn (both from Center for Biological Sequence Analysis, Technical University of Denmark DTU, http://www.cbs.dtu.dk/services/SignalP/background/prediction.php) for signal peptide prediction. The terms “signalp_hmm” and “signalp_nn” refer to two modes of operation for the program SignalP: hmm refers to Hidden Markov Model, while nn refers to neural networks. Localization was also determined through manual inspection of known protein localization and/or gene structure, and the use of heuristics by the individual inventor. In some cases for the manual inspection of cellular localization prediction inventors used the ProLoc computational platform [Einat Hazkani-Covo, Erez Levanon, Galit Rotman, Dan Graur and Amit Novik; (2004) “Evolution of multicellularity in metazoa: comparative analysis of the subcellular localization of proteins in Saccharomyces, Drosophila and Caenorhabditis.” Cell Biology International 2004; 28(3):171-8.], which predicts protein localization based on various parameters including, protein domains (e.g., prediction of trans-membranous regions and localization thereof within the protein), pI, protein length, amino acid composition, homology to pre-annotated proteins, recognition of sequence patterns which direct the protein to a certain organelle (such as, nuclear localization signal, NLS, mitochondria localization signal), signal peptide and anchor modeling and using unique domains from Pfam that are specific to a single compartment.

Information is given in the text with regard to SNPs (single nucleotide polymorphisms). A description of the abbreviations is as follows. “T->C”, for example, means that the SNP results in a change at the position given in the table from T to C. Similarly, “M->Q”, for example, means that the SNP has caused a change in the corresponding amino acid sequence, from methionine (M) to glutamine (Q). If, in place of a letter at the right hand side for the nucleotide sequence SNP, there is a space, it indicates that a frameshift has occurred. A frameshift may also be indicated with a hyphen (-). A stop codon is indicated with an asterisk at the right hand side (*). As part of the description of an SNP, a comment may be found in parentheses after the above description of the SNP itself. This comment may include an FTId, which is an identifier to a SwissProt entry that was created with the indicated SNP. An FTId is a unique and stable feature identifier, which allows construction of links directly from position-specific annotation in the feature table to specialized protein-related databases. The FTId is always the last component of a feature in the description field, as follows: FTId=XXX_number, in which XXX is the 3-letter code for the specific feature key, separated by an underscore from a 6-digit number. In the table of the amino acid mutations of the wild type proteins of the selected splice variants of the invention, the header of the first column is “SNP position(s) on amino acid sequence”, representing a position of a known mutation on amino acid sequence. SNPs may optionally be used as diagnostic markers according to the present invention, alone or in combination with one or more other SNPs and/or any other diagnostic marker. Preferred embodiments of the present invention comprise such SNPs, including but not limited to novel SNPs on the known (WT or wild type) protein sequences given below, as well as novel nucleic acid and/or amino acid sequences formed through such SNPs, and/or any SNP on a variant amino acid and/or nucleic acid sequence described herein.

Information given in the text with regard to the Homology to the known proteins was determined by Smith-Waterman version 5.1.2 using special (non default) parameters as follows:

model=sw.model

GAPEXT=0

GAPOP=100.0

MATRIX=blosum100

Information is given with regard to overexpression of a cluster in cancer based on ESTs. A key to the p values with regard to the analysis of such overexpression is as follows:
library-based statistics: P-value without including the level of expression in cell-lines (P1)
library based statistics: P-value including the level of expression in cell-lines (P2)
EST clone statistics: P-value without including the level of expression in cell-lines (SP1)
EST clone statistics: predicted overexpression ratio without including the level of expression in cell-lines (R3)
EST clone statistics: P-value including the level of expression in cell-lines (SP2)
EST clone statistics: predicted overexpression ratio including the level of expression in cell-lines (R4)

Library-based statistics refer to statistics over an entire library, while EST clone statistics refer to expression only for ESTs from a particular tissue or cancer.

Information is given with regard to overexpression of a cluster in cancer based on microarrays. As a microarray reference, in the specific segment paragraphs, the unabbreviated tissue name was used as the reference to the type of chip for which expression was measured. There are two types of microarray results: those from microarrays prepared according to a design by the present inventors, for which the microarray fabrication procedure is described in detail in Materials and Experimental Procedures section herein; and those results from microarrays using Affymetrix technology. As a microarray reference, in the specific segment paragraphs, the unabbreviated tissue name was used as the reference to the type of chip for which expression was measured. For microarrays prepared according to a design by the present inventors, the probe name begins with the name of the cluster (gene), followed by an identifying number. Oligonucleotide microarray results taken from Affymetrix data were from chips available from Affymetrix Inc, Santa Clara, Calif., USA (see for example data regarding the Human Genome U133 (HG-U133) Set at www.affymetrix.com/products/arrays/specific/hgu133.affx; GeneChip Human Genome U133A 2.0 Array at www.affymetrix.com/products/arrays/specific/hgu133av2.affx; and Human Genome U133 Plus 2.0 Array at www.affymetrix.com/products/arrays/specific/hgu133plus.affx). The probe names follow the Affymetrix naming convention. The data is available from NCBI Gene Expression Omnibus (see www.ncbi.nlm.nih.gov/projects/geo/and Edgar et al, Nucleic Acids Research, 2002, Vol. 30, No. 1 207-210). The dataset (including results) is available from www.ncbi.nlm.nih.gov/geo/query/acc.cgi?acc=GSE1133 for the Series GSE1133 database (published on March 2004); a reference to these results is as follows: Su et al (Proc Natl Acad Sci USA. 2004 Apr. 20; 101(16):6062-7. Epub 2004 Apr. 9). A list of probes is given below.

>M85491_0_0_25999

(SEQ ID NO: 1398)

GACATCTTTGCATATCATGTCAGAGCTATAACATCATTGTGGAGAAGCTC
>M85491_0_14_0

(SEQ ID NO: 1399)

GTCATGAAAATCAACACCGAGGTGCGGAGGTTCGGACCTGTGTCCCGCAG
>H53626_0_16_0

(SEQ ID NO: 1400)

ATGCGGGCATGTACATCTGCCTTGGCGCCAACACCATGGGCTACAGCTTC
>H53626_0_0_8391

(SEQ ID NO: 1401)

GGGTCTGGGGTGCTCTCCTGGTCTTTGTGTCGGCGTTCCCCTCCCTACCT
>HSENA78_0_1_0

(SEQ ID NO: 1402)

TGAAGAGTGTGAGGAAAACCTATGTTTGCCGCTTAAGCTTTCAGCTCAGC
>HUMGROG5_0_0_16626

(SEQ ID NO: 1403)

GCAGAAACTTTGCAGTAACACCTTCAGTGAGTTCAAGGCTAGGATCCCTG
>R00299_0_8_0

(SEQ ID NO: 1404)

CCAAGGCTCGTCTGCGCACCTTGTGTCTTGTAGGGTATGGTATGTGGGAC
>S67314_0_0_741

(SEQ ID NO: 1405)

CACAGAGCCAGGATGTTCTTCTGACCTCAGTATCTACTCCAGCTCCAGCT
>S67314_0_0_744

(SEQ ID NO: 1406)

TGGCATGCTGGAACATGGACTCTAGCTAGCAAGAAGGGCTCAAGGAGGTG
>Z44808_0_8_0

(SEQ ID NO: 1407)

AAAAGCATGAGTTTCTGACCAGCGTTCTGGACGCGCTGTCCACGGACATG
>Z44808_0_0_72347

(SEQ ID NO: 1408)

ATGTTCTTAGGAGGCAAGCCAGGAGAAGCCGGGTCTGACTTTTCAGCTCA
>Z44808_0_0_72349

(SEQ ID NO: 1409)

TCCTCCAGACCCAAAGCCACAACCCATCGCAAGTCAAGAACACTTTCCAG
>Z25299_0_3_0

(SEQ ID NO: 1410)

AACTCTGGCACCTTGGGCTGTGGAAGGCTCTGGAAAGTCCTTCAAAGCTG
>HUMCA1XIA_0_0_14909

(SEQ ID NO: 1411)

GCTGCAATCTAAGTTTCGGAATACTTATACCACTCCAGAAATAATCCTCG
>HUMCA1XIA_0_18_0

(SEQ ID NO: 1412)

TTCAGAACTGTTAACATCGCTGACGGGAAGTGGCATCGGGTAGCAATCAG
>HSS100PCB_0_0_12280

(SEQ ID NO: 1413)

CTCAAAATGAAACTCCCTCTCGCAGAGCACAATTCCAATTCGCTCTAAAA
>HUMPHOSLIP_0_0_18458

(SEQ ID NO: 1414)

AAGGAAGCAGGACCAGTGGATGTGAGGCGTGGTCGAAGAACAACAGAAAG
>HUMPHOSLIP_0_0_18487

(SEQ ID NO: 1415)

ACAGGGGCCAGATGGTGACCCATGACCCAGCCTAAAAGGCAGCCAGAGGG
>D11853_0_0_0

(SEQ ID NO: 1416)

GAGGCCCCTGGGTGGGAATGGGGACAGGAATTGACAGTGGAAGGGGTTCT
>D11853_0_0_3085

(SEQ ID NO: 1417)

TGACTCCCTACATACTCCAGGACTAGCTTAGGTCCCAACCCAATAGTTCC
>D11853_0_0_3082

(SEQ ID NO: 1418)

TGGTCCCCATGTGATTCTCCGAGGATCCTGAGGGTCGTGGTTTATGGAGA
>M77903_0_0_21402

(SEQ ID NO: 1419)

ACGTGATGGTTGGAACGCGTACCTTAGAGCTTCCAGTTCCGTCTTAGGAC
>AA583399_0_12_0

(SEQ ID NO: 1420)

ATCCCCACTGAACCCAGTGCTTTCACCAGCCATATTAGCTCCCACTCACC
>AA583399_0_0_1681

(SEQ ID NO: 1421)

CACCGCATGCTGCCAATCTGATGGTGGAGACAGAACAGCAGTCCCGGATG
>AA583399_0_1_1687

(SEQ ID NO: 1422)

TTTCCACACTCAGTGCCACGAAGTGCAGCTCTAAGCTGGGGATTTCTGTG
>HUMCACH1A_0_12_0

(SEQ ID NO: 1423)

ACCCAGCTCCATGTGCGTTCTCAGGGAATGGACGCCAGTGTACTGCCAAT
>HUMCACH1A_0_3_14917

(SEQ ID NO: 1331)

AGAGAATATCACTCCGATGGTCGGTTTCTGACTGTCACGCTAAGGGCAAC
>HUMGACH1A_0_0_14922

(SEQ ID NO: 1332)

GAACACAGAGAACGTCAGCGGTGAAGGCGAGAACCGAGGCTGCTGTGGAA
>HUMCACH1A_0_0_14913

(SEQ ID NO: 1334)

GACTCAGGAGATGAACAGCTCCCAACTATTTGCCGGGAAGACCCAGAGAT
>HUMCACH1A_0_0_14924

(SEQ ID NO: 1333)

GGCCCAGCATTGGGAACCTTGAGCATGTGTCTGAAAATGGGCATCATTCT
>HUMCEA_0_0_96

(SEQ ID NO: 1338)

CAAGAGGGGTTTGGCTGAGACTTTAGGATTGTGATTCAGCTTAGAGGGAC
>HUMCEA_0_0_15183

(SEQ ID NO: 1429)

CCTGGTGGGAGCCCATGAGAAGCGAGTTCTCTGTGCAACGGACTTAGTAA
>HUMCEA_0_0_15182

(SEQ ID NO: 1430)

GCTCCCTGGAGCATCAGCATCATATTCTGGGGTGGAGTCTATCTGGTTCT
>HUMCEA_0_0_15168

(SEQ ID NO: 1339)

TCCTGCCTGTCACCTGAAGTTCTAGATCATTCCCTGGACTCCACTCTATC
>HUMCEA_0_0_15180

(SEQ ID NO: 1432)

TTTAACACAGGATTGGGACAGGATTCAGAGGGACACTGTGGCCCTTCTAC
>M78035_0_0_21693

(SEQ ID NO: 1433)

CCATCCACATTTATGGAAACACTTGCTGTATATCTGGTGATTTACGTGTG
>M78035_0_0_21691

(SEQ ID NO: 1434)

CCTTTCACCACTGTGTGCAAGCGAATACACGCGGAACAATCCTAGTGAAT
>M78035_0_1_21707

(SEQ ID NO: 1435)

TTTGCTAGAAATCTGGTGTGGTGCAGGAGCGACTCCAGGATTCACTCTGT
>T23657_0_18_0

(SEQ ID NO: 1436)

TCCGTGACCCTCAGAGATCCTTTGCCCTGGGAATCCAGTGGATTGTAGTT
>T51958_0_0_50903

(SEQ ID NO: 1437)

CCCATGGTGGCCAGAGTGTCAGGTCTCATCGTGACGCTCTTGTCCTCCTC
>T51958_0_0_50916

(SEQ ID NO: 1438)

GGGGCTGTGCCCAGTCCCCCTGTCAGACCCTCAATGACTGAGGCCTGGGG
>Z17877_0_4_0

(SEQ ID NO: 1439)

ACTTTGCACTGGAACTTACAACACCCGAGCAAGGACGCGACTCTCCCGAC
>HSHCGI_0_0_10611

(SEQ ID NO: 1440)

GCCTACTGATTCATCCACATACAATTCTCAGCGTATATCCAAATGCAGTC
>HSHCGI_0_0_10620

(SEQ ID NO: 1441)

GGACCTCTAAGTCTACAGGTGGTCAAAATGCTGTATCCACCCAATTCCAC

The following list of abbreviations for tissues was used in the TAA histograms. The term “TAA” stands for “Tumor Associated Antigen”, and the TAA histograms, given in the text, represent the cancerous tissue expression pattern as predicted by the biomarkers selection engine, as described in detail in examples 1-5 below:
“BONE” for “bone”;
“COL” for “colon”;
“EPI” for “epithelial”;
“GEN” for “general”;
“LIVER” for “liver”;
“LUN” for “lung”;
“LYMPH” for “lymph nodes”;
“MARROW” for “bone marrow”;
“OVA” for “ovary”;
“PANCREAS” for “pancreas”;
“PRO” for “prostate”;
“STOMACH” for “stomach”;
“TCELL” for “T cells”;
“THYROID” for “Thyroid”;
“MAM” for “breast”;
“BRAIN” for “brain”;
“UTERUS” for “uterus”;
“SKIN” for “skin”;
“KIDNEY” for “kidney”;
“MUSCLE” for “muscle”;
“ADREN” for “adrenal”;
“HEAD” for “head and neck”;
“BLADDER” for “bladder”;

It should be noted that the terms “segment”, “seg” and “node” are used interchangeably in reference to nucleic acid sequences of the present invention; they refer to portions of nucleic acid sequences that were shown to have one or more properties as described below. They are also the building blocks that were used to construct complete nucleic acid sequences as described in greater detail below. Optionally and preferably, they are examples of oligonucleotides which are embodiments of the present invention, for example as amplicons, hybridization units and/or from which primers and/or complementary oligonucleotides may optionally be derived, and/or for any other use.

As used herein the phrase “colon cancer” refers to cancers of the colon or colorectal cancers.

The term “marker” in the context of the present invention refers to a nucleic acid fragment, a peptide, or a polypeptide, which is differentially present in a sample taken from subjects (patients) having colon cancer as compared to a comparable sample taken from subjects who do not have colon cancer.

The phrase “differentially present” refers to differences in the quantity of a marker present in a sample taken from patients having colon cancer as compared to a comparable sample taken from patients who do not have colon cancer. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the present invention can be effected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

As used herein, the term “level” refers to expression levels of RNA and/or protein or to DNA copy number of a marker of the present invention.

Typically the level of the marker in a biological sample obtained from the subject is different (i.e., increased or decreased) from the level of the same variant in a similar sample obtained from a healthy individual (examples of biological samples are described herein).

Numerous well known tissue or fluid collection methods can be utilized to collect the biological sample from the subject in order to determine the level of DNA, RNA and/or polypeptide of the variant of interest in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

Determining the level of the same variant in normal tissues of the same origin is preferably effected along-side to detect an elevated expression and/or amplification and/or a decreased expression, of the variant as opposed to the normal tissues.

A “test amount” of a marker refers to an amount of a marker in a subject's sample that is consistent with a diagnosis of colon cancer. A test amount can be either in absolute amount (e.g., microgram/ml) or a relative amount (e.g., relative intensity of signals).

A “control amount” of a marker can be any amount or a range of amounts to be compared against a test amount of a marker. For example, a control amount of a marker can be the amount of a marker in a patient with colon cancer or a person without colon cancer. A control amount can be either in absolute amount (e.g., microgram/ml) or a relative amount (e.g., relative intensity of signals).

“Detect” refers to identifying the presence, absence or amount of the object to be detected.

A “label” includes any moiety or item detectable by spectroscopic, photo chemical, biochemical, immunochemical, or chemical means. For example, useful labels include ³²P, ³⁵S, fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavadin. The label may be directly or indirectly detectable. Indirect detection can involve the binding of a second label to the first label, directly or indirectly. For example, the label can be the ligand of a binding partner, such as biotin, which is a binding partner for streptavadin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner, Bio/Technology 6:1165 (1988)). Quantitation of the signal is achieved by, e.g., scintillation counting, densitometry, or flow cytometry.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide (or other epitope), refers to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

According to preferred embodiments of the present invention, there is provided an isolated polynucleotide comprising a transcript SEQ ID NOs: 1 and 2.

According to preferred embodiments of the present invention, there is provided an isolated polynucleotide comprising a segment SEQ ID NOs: 89, 90, 91, 92, 93, 94, 95, 96, 97, 98 and 99. According to preferred embodiments of the present invention, there is provided an isolated polypeptide comprising SEQ ID NOs 534 and 535.

According to preferred embodiments of the present invention, there is provided an isolated polynucleotide comprising a transcript SEQ ID NOs: 3, 4, 5 and 6.

According to preferred embodiments of the present invention, there is provided an isolated polynucleotide comprising a segment SEQ ID NOs: 100, 101, 102, 103, 104, 105, 106 and 107.

According to preferred embodiments of the present invention, there is provided an isolated polypeptide comprising SEQ ID NOs 536, 537, 538 and 539.

According to preferred embodiments of the present invention, there is provided an isolated polynucleotide comprising a transcript SEQ ID NO. 7.

According to preferred embodiments of the present invention, there is provided an isolated polynucleotide comprising a segment SEQ ID NOs: 108, 109, 110, 111, 112, 113, 114, 115, 116, 117, 118, 119, 120, 121 and 122.

According to preferred embodiments of the present invention, there is provided an isolated polypeptide comprising SEQ ID NOs 540.

According to preferred embodiments of the present invention, there is provided an isolated polynucleotide comprising a transcript selected from the group consisting of SEQ ID NO. 8 and 9.

According to preferred embodiments of the present invention, there is provided an isolated polynucleotide comprising a segment selected from the group consisting of SEQ ID NOs: 123, 124, 125, 126, 127, 128, 129, 130, 131, 132, 133, 134, 135, 136, 137, 138, 139, 140, 141 and 142.

According to preferred embodiments of the present invention, there is provided an isolated polypeptide comprising SEQ ID NOs 541, 542.

According to preferred embodiments of the present invention, there is provided an isolated polynucleotide comprising a transcript SEQ ID NO. 10.

According to preferred embodiments of the present invention, there is provided an isolated polynucleotide comprising a segment SEQ ID NOs: 143, 144, 145, 146, 147, 148 and 149.

According to preferred embodiments of the present invention, there is provided an isolated polypeptide comprising SEQ ID NOs 543.

According to preferred embodiments of the present invention, there is provided an isolated polynucleotide comprising a transcript SEQ ID NO. 11, 12, 13 and 14.

According to preferred embodiments of the present invention, there is provided an isolated polynucleotide comprising a segment SEQ ID NOs: 150, 151, 152, 153, 154, 155, 156, 157, 158, 159, 160, 161, 162, 163, 164, 165, 166 and 167.

According to preferred embodiments of the present invention, there is provided an isolated polypeptide comprising SEQ ID NOs 544, 545, 546 and 547.

According to preferred embodiments of the present invention, there is provided an isolated polynucleoide comprising a transcript SEQ ID NO. 15.

According to preferred embodiments of the present invention, there is provided an isolated polynucleotide comprising a segment SEQ ID NOs: 168, 169, 170, 171, 172, 173, 174, 175, 176, 177, 178, 179, 180, 181, 182, 183 and 184.

According to preferred embodiments of the present invention, there is provided an isolated polypeptide comprising SEQ ID NO. 548.

According to preferred embodiments of the present invention, there is provided an isolated polynucleotide comprising a transcript SEQ ID NO. 16.

According to preferred embodiments of the present invention, there is provided an isolated polynucleotide comprising a segment SEQ ID NOs: 185, 186, 187, 188, 189, 190, 191, 192, 193, 194, 195 and 196.

According to preferred embodiments of the present invention, there is provided an isolated polypeptide comprising SEQ ID NOs 549.

According to preferred embodiments of the present invention, there is provided an isolated polynucleotide comprising a transcript SEQ ID NO. 17 and 18.

According to preferred embodiments of the present invention, there is provided an isolated polynucleotide comprising a segment SEQ ID NOs: 197, 198, 199, 200, 201, 202, 203, 204, 205, 206, 207, 208, 209, 210 and 211.

According to preferred embodiments of the present invention, there is provided an isolated polypeptide comprising SEQ ID NOs 550 and 551.

According to preferred embodiments of the present invention, there is provided an isolated polynucleotide comprising a transcript SEQ ID NO. 19, 20, 21 and 22.

According to preferred embodiments of the present invention, there is provided an isolated polynucleotide comprising a segment SEQ ID NOs: 212, 213, 214, 215, 216, 217, 218 and 219.

According to preferred embodiments of the present invention, there is provided an isolated polypeptide comprising SEQ ID NOs 552, 553, 554 and 555.

According to preferred embodiments of the present invention, there is provided an isolated polynucleotide comprising a transcript SEQ ID NO. 23, 24, 25, 26 and 27.

According to preferred embodiments of the present invention, there is provided an isolated polynucleotide comprising a segment SEQ ID NOs: 220, 221, 222, 223, 224, 225, 226, 227, 228, 229, 230, 231, 232, 233, 234, 235, 236, 237, 238, 239 and 240.

According to preferred embodiments of the present invention, there is provided an isolated polypeptide comprising SEQ ID NOs 556, 557, 558 and 559.

According to preferred embodiments of the present invention, there is provided an isolated polynucleotide comprising a transcript SEQ ID NO. 28, 29, 30, 31 and 32.

According to preferred embodiments of the present invention, there is provided an isolated polynucleotide comprising a segment SEQ ID NOs: 241, 242, 243, 244, 245, 246, 247, 248, 249, 250 and 251.

According to preferred embodiments of the present invention, there is provided an isolated polypeptide comprising SEQ ID NOs 560, 561, 562 and 563.

According to preferred embodiments of the present invention, there is provided an isolated polynucleotide comprising a transcript SEQ ID NO. 33, 34, and 35.

According to preferred embodiments of the present invention, there is provided an isolated polynucleotide comprising a segment SEQ ID NOs: 252, 253, 254, 255, 256, 257, 258, 259, 260, 261, 262, 263, 264, 265, 266, 267, 267, 269, 270, 271, 272, 273, 274, 275, 276, 277, 278, 279, 280, 281, 282, 283, 284.

According to preferred embodiments of the present invention, there is provided an isolated polypeptide comprising SEQ ID NOs 564, 565, and 566.

According to preferred embodiments of the present invention, there is provided an isolated polynucleotide comprising a transcript SEQ ID NO. 36, 37, 38, 39, 40, 41, 42 and 43.

According to preferred embodiments of the present invention, there is provided an isolated polynucleotide comprising a segment SEQ ID NOs: 285, 286, 287, 288, 289, 290, 291, 292, 293, 294, 295, 296, 297, 298, 299, 300, 301, 302, 303, 304, 305 and 306.

According to preferred embodiments of the present invention, there is provided an isolated polypeptide comprising SEQ ID NOs 567, 568, 569, 570, 571, 572, 573 and 574.

According to preferred embodiments of the present invention, there is provided an isolated polynucleotide comprising a transcript SEQ ID NO. 44.

According to preferred embodiments of the present invention, there is provided an isolated polynucleotide comprising a segment SEQ ID NOs: 307, 308, 309, 310, 311, 312, 313, 314, 315 and 316.

According to preferred embodiments of the present invention, there is provided an isolated polypeptide comprising SEQ ID NO. 575.

According to preferred embodiments of the present invention, there is provided an isolated polynucleotide comprising a transcript SEQ ID NO. 45, 46, 47 and 48.

According to preferred embodiments of the present invention, there is provided an isolated polynucleotide comprising a segment SEQ ID NOs: 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361 and 362.

According to preferred embodiments of the present invention, there is provided an isolated polypeptide comprising SEQ ID NOs 576, 577, 578 and 579.

According to preferred embodiments of the present invention, there is provided an isolated polynucleotide comprising a transcript SEQ ID NO. 49.

According to preferred embodiments of the present invention, there is provided an isolated polynucleotide comprising a segment SEQ ID NOs: 363, 364 and 365.

According to preferred embodiments of the present invention, there is provided an isolated polypeptide comprising SEQ ID NO. 580.

According to preferred embodiments of the present invention, there is provided an isolated polynucleotide comprising a transcript SEQ ID NO. 50, 51, 52, 53, 54, 55 and 56.

According to preferred embodiments of the present invention, there is provided an isolated polynucleotide comprising a segment SEQ ID NOs: 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417 and 418.

According to preferred embodiments of the present invention, there is provided an isolated polypeptide comprising SEQ ID NOs 581, 582, 583, 584, 585, 586 and 587.

According to preferred embodiments of the present invention, there is provided an isolated polynucleotide comprising a transcript SEQ ID NO. 57, 58, 59, 60, 61, 62, 63, 64, 65, 66, 67, 68, 69, 70, 71, 72 , 73 and 74.

According to preferred embodiments of the present invention, there is provided an isolated polynucleotide comprising a segment SEQ ID NOs: 419, 420, 421, 422, 423, 424, 425, 426, 427, 428, 429, 43, 431, 432, 433, 434, 435, 436, 437, 438, 439, 440, 441, 442, 443, 444, 445, 446, 447, 448 and 449.

According to preferred embodiments of the present invention, there is provided an isolated polypeptide comprising SEQ ID NOs 588, 589, 590, 591, 592, 593, 594, 595, 596, 597, 598, 599, 600, 601 and 602.

According to preferred embodiments of the present invention, there is provided an isolated polynucleotide comprising a transcript SEQ ID NO. 75, 76, 77, 78, 79 and 80.

According to preferred embodiments of the present invention, there is provided an isolated polynucleotide comprising a segment SEQ ID NOs: 450, 451, 452, 453, 454, 455, 456, 457, 458, 459, 460, 461, 462, 463, 464, 465, 466, 467, 468, 469, 470, 471, 472, 473, 474 and 475.

According to preferred embodiments of the present invention, there is provided an isolated polypeptide comprising SEQ ID NOs 603, 604, 605, 606 and 607.

According to preferred embodiments of the present invention, there is provided an isolated polynucleotide comprising a transcript SEQ ID NO. 81, 82, 83 and 84.

According to preferred embodiments of the present invention, there is provided an isolated polynucleotide comprising a segment SEQ ID NOs: 476, 477, 478, 479, 480, 481, 482, 483, 484, 485, 486, 487, 488, 489, 490, 491, 492, 493, 494, 495, 496, 497, 498, 499, 500, 501, 502, 503 and 504.

According to preferred embodiments of the present invention, there is provided an isolated polypeptide comprising SEQ ID NOs 608, 609, 610 and 611.

According to preferred embodiments of the present invention, there is provided an isolated polynucleotide comprising a transcript SEQ ID NO. 85, 86, 87 and 88.

According to preferred embodiments of the present invention, there is provided an isolated polynucleotide comprising a segment SEQ ID NOs: 505-532 and 533.

According to preferred embodiments of the present invention, there is provided an isolated polypeptide comprising SEQ ID NOs: 612, 613, 614 and 615.

According to preferred embodiments of the present invention, there is provided an isolated chimeric polypeptide encodings from clusters M85491, T10888, H14624, H53626, HSENA78, HUMGROG5, HUMODCA, R00299, Z19178, S67314, Z44808, Z25299, HUMF5A, HUMANK, Z39818, HUMCA1XIA, HSS100PCB, HUMPHOSLIP, D11853, R11723, M77903 and HSKITCR.

According to preferred embodiments of the present invention, there is provided an isolated chimeric polypeptide encoding for SEQ ID NO. 608, comprising a first amino acid sequence being at least 90% homologous to amino acids 1-207 of SSRA_HUMAN (SEQ ID NO:641), which also corresponds to amino acids 1-207 of SEQ ID NO. 608, and a second amino acid sequence being at least 70%, optionally at least 80%, preferably at least 85%, more preferably at least 90% and most preferably at least 95% homologous to a polypeptide corresponding to amino acids 208-214 of SEQ ID NO. 608, wherein said first amino acid sequence and second amino acid sequence are contiguous and in a sequential order.

According to preferred embodiments of the present invention, there is provided an isolated polypeptide encoding for a tail of SEQ ID NO. 608, comprising a polypeptide being at least 70%, optionally at least about 80%, preferably at least about 85%, more preferably at least about 90% and most preferably at least about 95% homologous to in SEQ ID NO. 608.

According to preferred embodiments of the present invention, there is provided an isolated chimeric polypeptide encoding for SEQ ID NO. 609, comprising a first amino acid sequence being at least 90% homologous to amino acids 1-207 of SSRA_HUMAN (SEQ ID NO:641), which also corresponds to amino acids 1-207 of SEQ ID NO. 609.

According to preferred embodiments of the present invention, there is provided an isolated chimeric polypeptide encoding for SEQ ID NO. 610, comprising a first amino acid sequence being at least 90% homologous to amino acids 1-181 of SSRA_HUMAN (SEQ ID NO:641), which also corresponds to amino acids 1-181 of SEQ ID NO. 610, and a second amino acid sequence being at least 70%, optionally at least 80%, preferably at least 85%, more preferably at least 90% and most preferably at least 95% homologous to a polypeptide corresponding to amino acids 182-192 of SEQ ID NO. 610, wherein said first amino acid sequence and second amino acid sequence are contiguous and in a sequential order.

According to preferred embodiments of the present invention, there is provided an isolated polypeptide encoding for a tail of SEQ ID NO. 610, comprising a polypeptide being at least 70%, optionally at least about 80%, preferably at least about 85%, more preferably at least about 90% and most preferably at least about 95% homologous to in SEQ ID NO. 610.

According to preferred embodiments of the present invention, there is provided an isolated chimeric polypeptide encoding for SEQ ID NO. 611, comprising a first amino acid sequence being at least 90% homologous to amino acids 1-93 of SSRA_HUMAN (SEQ ID NO:641), which also corresponds to amino acids 1-93 of SEQ ID NO. 611, and a second amino acid sequence being at least 70%, optionally at least 80%, preferably at least 85%, more preferably at least 90% and most preferably at least 95% homologous to a polypeptide corresponding to amino acids 94-104 of SEQ ID NO. 611, wherein said first amino acid sequence and second amino acid sequence are contiguous and in a sequential order.

According to preferred embodiments of the present invention, there is provided an isolated polypeptide encoding for a tail of SEQ ID NO. 611, comprising a polypeptide being at least 70%, optionally at least about 80%, preferably at least about 85%, more preferably at least about 90% and most preferably at least about 95% homologous to amino in SEQ ID NO. 611.

According to preferred embodiments of the present invention, there is provided an isolated chimeric polypeptide encoding for SEQ ID NO. 604, comprising a first amino acid sequence being at least 70%, optionally at least 80%, preferably at least 85%, more preferably at least 90% and most preferably at least 95% homologous to a polypeptide corresponding to amino acids 1-110 of SEQ ID NO. 604, and a second amino acid sequence being at least 90% homologous to amino acids 1-112 of Q8IXM0, which also corresponds to amino acids 111-222 of SEQ ID NO. 604, wherein said first and second amino acid sequences are contiguous and in a sequential order.

According to preferred embodiments of the present invention, there is provided an isolated polypeptide encoding for a head of SEQ ID NO. 604, comprising a polypeptide being at least 70%, optionally at least about 80%, preferably at least about 85%, more preferably at least about 90% and most preferably at least about 95% homologous to amino acids 1-110 of SEQ ID NO. 604.

According to preferred embodiments of the present invention, there is provided an isolated chimeric polypeptide encoding for SEQ ID NO. 604, comprising a first amino acid sequence being at least 90% homologous to amino acids 1-83 of Q96AC2, which also corresponds to amino acids 1-83 of SEQ ID NO. 604, and a second amino acid sequence being at least 70%, optionally at least 80%, preferably at least 85%, more preferably at least 90% and most preferably at least 95% homologous to a polypeptide corresponding to amino acids 84-222 of SEQ ID NO. 604, wherein said first and second amino acid sequences are contiguous and in a sequential order.

According to preferred embodiments of the present invention, there is provided an isolated polypeptide encoding for a tail of SEQ ID NO. 604, comprising a polypeptide being at least 70%, optionally at least about 80%, preferably at least about 85%, more preferably at least about 90% and most preferably at least about 95% homologous to amino in SEQ ID NO. 604.

According to preferred embodiments of the present invention, there is provided an isolated chimeric polypeptide encoding for SEQ ID NO. 604, comprising a first amino acid sequence being at least 90% homologous to amino acids 1-83 of Q8N2G4, which also corresponds to amino acids 1-83 of SEQ ID NO. 604, and a second amino acid sequence being at least 70%, optionally at least 80%, preferably at least 85%, more preferably at least 90% and most preferably at least 95% homologous to a polypeptide corresponding to amino acids 84-222 of SEQ ID NO. 604, wherein said first and second amino acid sequences are contiguous and in a sequential order.

According to preferred embodiments of the present invention, there is provided an isolated polypeptide encoding for a tail of SEQ ID NO. 604, comprising a polypeptide being at least 70%, optionally at least about 80%, preferably at least about 85%, more preferably at least about 90% and most preferably at least about 95% homologous to amino in SEQ ID NO. 604.

According to preferred embodiments of the present invention, there is provided an isolated chimeric polypeptide encoding for SEQ ID NO. 604, comprising a first amino acid sequence being at least 90% homologous to amino acids 24-106 of BAC85518 (SEQ ID NO:1396), which also corresponds to amino acids 1-83 of SEQ ID NO. 604, and a second amino acid sequence being at least 70%, optionally at least 80%, preferably at least 85%, more preferably at least 90% and most preferably at least 95% homologous to a polypeptide corresponding to amino acids 84-222 of SEQ ID NO. 604, wherein said first and second amino acid sequences are contiguous and in a sequential order.

According to preferred embodiments of the present invention, there is provided an isolated polypeptide encoding for a tail of SEQ ID NO. 604, comprising a polypeptide being at least 70%, optionally at least about 80%, preferably at least about 85%, more preferably at least about 90% and most preferably at least about 95% homologous to amino in SEQ ID NO. 604.

According to preferred embodiments of the present invention, there is provided an isolated chimeric polypeptide encoding for SEQ ID NO. 605, comprising a first amino acid sequence being at least 90% homologous to amino acids 1-64 of Q96AC2, which also corresponds to amino acids 1-64 of SEQ ID NO. 605, and a second amino acid sequence being at least 70%, optionally at least 80%, preferably at least 85%, more preferably at least 90% and most preferably at least 95% homologous to a polypeptide corresponding to amino acids 65-93 of SEQ ID NO. 605, wherein said first and second amino acid sequences are contiguous and in a sequential order.

According to preferred embodiments of the present invention, there is provided an isolated polypeptide encoding for a tail of SEQ ID NO. 605, comprising a polypeptide being at least 70%, optionally at least about 80%, preferably at least about 85%, more preferably at least about 90% and most preferably at least about 95% homologous to amino acids 65-93 in SEQ ID NO. 605.

According to preferred embodiments of the present invention, there is provided an isolated chimeric polypeptide encoding for SEQ ID NO. 605, comprising a first amino acid sequence being at least 90% homologous to amino acids 1-64 of Q8N2G4, which also corresponds to amino acids 1-64 of SEQ ID NO. 605, and a second amino acid sequence being at least 70%, optionally at least 80%, preferably at least 85%, more preferably at least 90% and most preferably at least 95% homologous to a polypeptide corresponding to amino acids 65-93 of SEQ ID NO. 605, wherein said first and second amino acid sequences are contiguous and in a sequential order.

According to preferred embodiments of the present invention, there is provided an isolated polypeptide encoding for a tail of SEQ ID NO. 605, comprising a polypeptide being at least 70%, optionally at least about 80%, preferably at least about 85%, more preferably at least about 90% and most preferably at least about 95% homologous to amino acids 65-93 in SEQ ID NO. 605.

According to preferred embodiments of the present invention, there is provided an isolated chimeric polypeptide encoding for SEQ ID NO. 605, comprising a first amino acid sequence being at least 70%, optionally at least 80%, preferably at least 85%, more preferably at least 90% and most preferably at least 95% homologous to a polypeptide having the sequence MWVLG corresponding to amino acids 1-5 of SEQ ID NO. 605, second amino acid sequence being at least 90% homologous to amino acids 22-80 of BAC85273 (SEQ ID NO:1397), which also corresponds to amino acids 6-64 of SEQ ID NO. 605, and a third amino acid sequence being at least 70%, optionally at least 80%, preferably at least 85%, more preferably at least 90% and most preferably at least 95% homologous to a polypeptide sequence corresponding to amino acids 65-93 of SEQ ID NO. 605, wherein said first, second and third amino acid sequences are contiguous and in a sequential order.

According to preferred embodiments of the present invention, there is provided an isolated polypeptide encoding for a head of SEQ ID NO. 605, comprising a polypeptide being at least 70%, optionally at least about 80%, preferably at least about 85%, more preferably at least about 90% and most preferably at least about 95% homologous to amino acids 1-5 of SEQ ID NO. 605.

According to preferred embodiments of the present invention, there is provided an isolated polypeptide encoding for a tail of SEQ ID NO. 605, comprising a polypeptide being at least 70%, optionally at least about 80%, preferably at least about 85%, more preferably at least about 90% and most preferably at least about 95% homologous to amino acids 65-93 in SEQ ID NO. 605.

According to preferred embodiments of the present invention, there is provided an isolated chimeric polypeptide encoding for SEQ ID NO. 605, comprising a first amino acid sequence being at least 90% homologous to amino acids 24-87 of BAC85518 (SEQ ID NO:1396), which also corresponds to amino acids 1-64 of SEQ ID NO. 605, and a second amino acid sequence being at least 70%, optionally at least 80%, preferably at least 85%, more preferably at least 90% and most preferably at least 95% homologous to a polypeptide sequence corresponding to amino acids 65-93 of SEQ ID NO. 605, wherein said first and second amino acid sequences are contiguous and in a sequential order.

According to preferred embodiments of the present invention, there is provided an isolated polypeptide encoding for a tail of SEQ ID NO. 605, comprising a polypeptide being at least 70%, optionally at least about 80%, preferably at least about 85%, more preferably at least about 90% and most preferably at least about 95% homologous to amino acids 65-93 in SEQ ID NO. 605.

According to preferred embodiments of the present invention, there is provided an isolated chimeric polypeptide encoding for SEQ ID NO. 605, comprising a first amino acid sequence being at least 90% homologous to amino acids 1-63 of Q96AC2, which also corresponds to amino acids 1-63 of SEQ ID NO. 606, and a second amino acid sequence being at least 70%, optionally at least 80%, preferably at least 85%, more preferably at least 90% and most preferably at least 95% homologous to a polypeptide sequence corresponding to amino acids 64-84 of SEQ ID NO. 606, wherein said first and second amino acid sequences are contiguous and in a sequential order.

According to preferred embodiments of the present invention, there is provided an isolated polypeptide encoding for a tail of SEQ ID NO. 606, comprising a polypeptide being at least 70%, optionally at least about 80%, preferably at least about 85%, more preferably at least about 90% and most preferably at least about 95% homologous to amino acids 64-84 in SEQ ID NO. 606.

According to preferred embodiments of the present invention, there is provided an isolated chimeric polypeptide encoding for SEQ ID NO. 607, comprising a first amino acid sequence being at least 90% homologous to amino acids 1-63 of Q96AC2, which also corresponds to amino acids 1-63 of SEQ ID NO. 607, and a second amino acid sequence being at least 70%, optionally at least 80%, preferably at least 85%, more preferably at least 90% and most preferably at least 95% homologous to a polypeptide sequence corresponding to amino acids 64-90 of SEQ ID NO. 607, wherein said first and second amino acid sequences are contiguous and in a sequential order.

According to preferred embodiments of the present invention, there is provided an isolated polypeptide encoding for a tail of SEQ ID NO. 607, comprising a polypeptide being at least 70%, optionally at least about 80%, preferably at least about 85%, more preferably at least about 90% and most preferably at least about 95% homologous to amino acids 64-90 in SEQ ID NO. 607.

According to preferred embodiments of the present invention, there is provided an isolated chimeric polypeptide encoding for SEQ ID NO. 607, comprising a first amino acid sequence being at least 90% homologous to amino acids 1-63 of Q8N2G4, which also corresponds to amino acids 1-63 of SEQ ID NO. 607, and a second amino acid sequence being at least 70%, optionally at least 80%, preferably at least 85%, more preferably at least 90% and most preferably at least 95% homologous to a polypeptide sequence corresponding to amino acids 64-90 of SEQ ID NO. 607 wherein said first and second amino acid sequences are contiguous and in a sequential order.

According to preferred embodiments of the present invention, there is provided an isolated polypeptide encoding for a tail of SEQ ID NO. 607, comprising a polypeptide being at least 70%, optionally at least about 80%, preferably at least about 85%, more preferably at least about 90% and most preferably at least about 95% homologous to amino acids 64-90 in SEQ ID NO. 607.

According to preferred embodiments of the present invention, there is provided an isolated chimeric polypeptide encoding for SEQ ID NO. 607, comprising a first amino acid sequence being at least 70%, optionally at least 80%, preferably at least 85%, more preferably at least 90% and most preferably at least 95% homologous to a polypeptide sequence corresponding to amino acids 1-5 of SEQ ID NO. 607, second amino acid sequence being at least 90% homologous to amino acids 22-79 of BAC85273 (SEQ ID NO:1397), which also corresponds to amino acids 6-63 of SEQ ID NO. 607, and a third amino acid sequence being at least 70%, optionally at least 80%, preferably at least 85%, more preferably at least 90% and most preferably at least 95% homologous to a polypeptide sequence corresponding to amino acids 64-90 of SEQ ID NO. 607, wherein said first, second and third amino acid sequences are contiguous and in a sequential order.

According to preferred embodiments of the present invention, there is provided an isolated polypeptide encoding for a head of SEQ ID NO. 607, comprising a polypeptide being at least 70%, optionally at least about 80%, preferably at least about 85%, more preferably at least about 90% and most preferably at least about 95% homologous to amino acids 1-5 of SEQ ID NO. 607.

According to preferred embodiments of the present invention, there is provided an isolated polypeptide encoding for a tail of SEQ ID NO. 607, comprising a polypeptide being at least 70%, optionally at least about 80%, preferably at least about 85%, more preferably at least about 90% and most preferably at least about 95% homologous to amino acids 64-90 in SEQ ID NO. 607.

According to preferred embodiments of the present invention, there is provided an isolated chimeric polypeptide encoding for SEQ ID NO. 607, comprising a first amino acid sequence being at least 90% homologous to amino acids 24-86 of BAC85518 (SEQ ID NO:1396), which also corresponds to amino acids 1-63 of SEQ ID NO. 607, and a second amino acid sequence being at least 70%, optionally at least 80%, preferably at least 85%, more preferably at least 90% and most preferably at least 95% homologous to a polypeptide sequence corresponding to amino acids 64-90 of SEQ ID NO. 607, wherein said first and second amino acid sequences are contiguous and in a sequential order.

According to preferred embodiments of the present invention, there is provided an isolated polypeptide encoding for a tail of SEQ ID NO. 607, comprising a polypeptide being at least 70%, optionally at least about 80%, preferably at least about 85%, more preferably at least about 90% and most preferably at least about 95% homologous to amino acids 64-90 in SEQ ID NO. 607.

According to preferred embodiments of the present invention, there is provided an isolated chimeric polypeptide encoding for SEQ ID NO. 588, comprising a first amino acid sequence being at least 70%, optionally at least 80%, preferably at least 85%, more preferably at least 90% and most preferably at least 95% homologous to a polypeptide sequence corresponding to amino acids 1-26 of SEQ ID NO. 588, a second amino acid sequence being at least 90% homologous to amino acids 13-187 of SEQ ID NO. 639, which also corresponds to amino acids 27-201 of SEQ ID NO. 588, a bridging amino acid A corresponding to amino acid 202 of SEQ ID NO. 588, and a third amino acid sequence being at least 90% homologous to amino acids 189-342 of SEQ ID NO. 639, which also corresponds to amino acids 203-356 of SEQ ID NO. 588, wherein said first amino acid sequence, second amino acid sequence, bridging amino acid and third amino acid sequence are contiguous and in a sequential order.

According to preferred embodiments of the present invention, there is provided an isolated polypeptide encoding for a head of SEQ ID NO. 588, comprising a polypeptide being at least 70%, optionally at least about 80%, preferably at least about 85%, more preferably at least about 90% and most preferably at least about 95% homologous to the sequence amino acids 1-26 of SEQ ID NO. 588.

According to preferred embodiments of the present invention, there is provided an isolated chimeric polypeptide encoding for SEQ ID NO. 588, comprising a first amino acid sequence being at least 70%, optionally at least 80%, preferably at least 85%, more preferably at least 90% and most preferably at least 95% homologous to a polypeptide sequence corresponding to amino acids 1-109 of SEQ ID NO. 588, a second amino acid sequence being at least 90% homologous to amino acids 1-159 of SEQ ID NO. 640, which also corresponds to amino acids 110-268 of SEQ ID NO. 588, and a third amino acid sequence being at least 70%, optionally at least 80%, preferably at least 85%, more preferably at least 90% and most preferably at least 95% homologous to a polypeptide sequence corresponding to amino acids 269-356 of SEQ ID NO. 588, wherein said first amino acid sequence, second amino acid sequence and third amino acid sequence are contiguous and in a sequential order.

According to preferred embodiments of the present invention, there is provided an isolated polypeptide encoding for a head of SEQ ID NO. 588, comprising a polypeptide being at least 70%, optionally at least about 80%, preferably at least about 85%, more preferably at least about 90% and most preferably at least about 95% homologous to amino acids 269-356 of SEQ ID NO. 588.

According to preferred embodiments of the present invention, there is provided an isolated polypeptide encoding for a tail of SEQ ID NO. 588, comprising a polypeptide being at least 70%, optionally at least about 80%, preferably at least about 85%, more preferably at least about 90% and most preferably at least about 95% homologous to amino in SEQ ID NO. 588.

According to preferred embodiments of the present invention, there is provided an isolated chimeric polypeptide encoding for SEQ ID NO. 588, comprising a first amino acid sequence being at least 90% homologous to amino acids 1-128 of SEQ ID NO. 638, which also corresponds to amino acids 1-128 of SEQ ID NO. 588, a bridging amino acid L corresponding to amino acid 129 of SEQ ID NO. 588, and a second amino acid sequence being at least 90% homologous to amino acids 130-356 of SEQ ID NO. 638, which also corresponds to amino acids 130-356 of SEQ ID NO. 588, wherein said first amino acid sequence, bridging amino acid and second amino acid sequence are contiguous and in a sequential order.

According to preferred embodiments of the present invention, there is provided an isolated chimeric polypeptide encoding for SEQ ID NO. 589, comprising a first amino acid sequence being at least 70%, optionally at least 80%, preferably at least 85%, more preferably at least 90% and most preferably at least 95% homologous to amino acids 1-26 of SEQ ID NO. 589, a second amino acid sequence being at least 90% homologous to amino acids 13-187 of SEQ ID NO. 639, which also corresponds to amino acids 27-201 of SEQ ID NO. 589, a bridging amino acid A corresponding to amino acid 202 of SEQ ID NO. 589, a third amino acid sequence being at least 90% homologous to amino acids 189-297 of SEQ ID NO. 639, which also corresponds to amino acids 203-311 of SEQ ID NO. 589, and a fourth amino acid sequence being at least 70%, optionally at least 80%, preferably at least 85%, more preferably at least 90% and most preferably at least 95% homologous to a polypeptide sequence corresponding to amino acids 312-315 of SEQ ID NO. 589, wherein said first amino acid sequence, second amino acid sequence, bridging amino acid, third amino acid sequence and fourth amino acid sequence are contiguous and in a sequential order.

According to preferred embodiments of the present invention, there is provided an isolated polypeptide encoding for a head of SEQ ID NO. 589, comprising a polypeptide being at least 70%, optionally at least about 80%, preferably at least about 85%, more preferably at least about 90% and most preferably at least about 95% homologous to amino acids 1-26 of SEQ ID NO. 589.

According to preferred embodiments of the present invention, there is provided an isolated polypeptide encoding for a tail of SEQ ID NO. 589, comprising a polypeptide being at least 70%, optionally at least about 80%, preferably at least about 85%, more preferably at least about 90% and most preferably at least about 95% homologous to amino in SEQ ID NO. 589.

According to preferred embodiments of the present invention, there is provided an isolated chimeric polypeptide encoding for SEQ ID NO. 589, comprising a first amino acid sequence being at least 70%, optionally at least 80%, preferably at least 85%, more preferably at least 90% and most preferably at least 95% homologous to a polypeptide sequence to amino acids 1-109 of SEQ ID NO. 589, a second amino acid sequence being at least 90% homologous to amino acids 1-159 of SEQ ID NO. 640, which also corresponds to amino acids 110-268 of SEQ ID NO. 589, and a third amino acid sequence being at least 70%, optionally at least 80%, preferably at least 85%, more preferably at least 90% and most preferably at least 95% homologous to a polypeptide sequence corresponding to amino acids 269-315 of SEQ ID NO. 589, wherein said first amino acid sequence, second amino acid sequence and third amino acid sequence are contiguous and in a sequential order.

According to preferred embodiments of the present invention, there is provided an isolated polypeptide encoding for a head of SEQ ID NO. 589, comprising a polypeptide being at least 70%, optionally at least about 80%, preferably at least about 85%, more preferably at least about 90% and most preferably at least about 95% homologous to amino acids 1-109 of SEQ ID NO. 589.

According to preferred embodiments of the present invention, there is provided an isolated polypeptide encoding for a tail of SEQ ID NO. 589, comprising a polypeptide being at least 70%, optionally at least about 80%, preferably at least about 85%, more preferably at least about 90% and most preferably at least about 95% homologous to amino in SEQ ID NO. 589.

According to preferred embodiments of the present invention, there is provided an isolated chimeric polypeptide encoding for SEQ ID NO. 589, comprising a first amino acid sequence being at least 90% homologous to amino acids 1-128 of SEQ ID NO. 638, which also corresponds to amino acids 1-128 of SEQ ID NO. 589, a bridging amino acid L corresponding to amino acid 129 of SEQ ID NO. 589, a second amino acid sequence being at least 90% homologous to amino acids 130-311 of SEQ ID NO. 638, which also corresponds to amino acids 130-311 of SEQ ID NO. 589, and a third amino acid sequence being at least 70%, optionally at least 80%, preferably at least 85%, more preferably at least 90% and most preferably at least 95% homologous to a polypeptide sequence corresponding to amino acids 130-311 of SEQ ID NO. 589, wherein said first amino acid sequence, bridging amino acid, second amino acid sequence and third amino acid sequence are contiguous and in a sequential order.

According to preferred embodiments of the present invention, there is provided an isolated polypeptide encoding for a tail of SEQ ID NO. 589, comprising a polypeptide being at least 70%, optionally at least about 80%, preferably at least about 85%, more preferably at least about 90% and most preferably at least about 95% homologous to amino in SEQ ID NO. 589.

According to preferred embodiments of the present invention, there is provided an isolated chimeric polypeptide encoding for SEQ ID NO. 589, comprising a first amino acid sequence being at least 90% homologous to amino acids 1-311 of Q9UJZ1 (SEQ ID NO:637), which also corresponds to amino acids 1-311 of SEQ ID NO. 589, and a second amino acid sequence being at least 70%, optionally at least 80%, preferably at least 85%, more preferably at least 90% and most preferably at least 95% homologous to a polypeptide sequence corresponding to amino acids 312-315 of SEQ ID NO. 589, wherein said first amino acid sequence and second amino acid sequence are contiguous and in a sequential order.

According to preferred embodiments of the present invention, there is provided an isolated polypeptide encoding for a tail of SEQ ID NO. 589, comprising a polypeptide being at least 70%, optionally at least about 80%, preferably at least about 85%, more preferably at least about 90% and most preferably at least about 95% homologous to amino in SEQ ID NO. 589.

According to preferred embodiments of the present invention, there is provided an isolated chimeric polypeptide encoding for SEQ ID NO. 589, comprising a first amino acid sequence being at least 70%, optionally at least 80%, preferably at least 85%, more preferably at least 90% and most preferably at least 95% homologous to a polypeptide sequence corresponding to amino acids 1-109 of SEQ ID NO. 589, a second amino acid sequence being at least 90% homologous to amino acids 1-159 of SEQ ID NO. 640, which also corresponds to amino acids 110-268 of SEQ ID NO. 589, and a third amino acid sequence being at least 70%, optionally at least 80%, preferably at least 85%, more preferably at least 90% and most preferably at least 95% homologous to a polypeptide sequence corresponding to amino acids 269-315 of SEQ ID NO. 589, wherein said first amino acid sequence, second amino acid sequence and third amino acid sequence are contiguous and in a sequential order.

According to preferred embodiments of the present invention, there is provided an isolated polypeptide encoding for a head of SEQ ID NO. 589, comprising a polypeptide being at least 70%, optionally at least about 80%, preferably at least about 85%, more preferably at least about 90% and most preferably at least about 95% homologous to amino acids 1-109 of SEQ ID NO. 589.

According to preferred embodiments of the present invention, there is provided an isolated polypeptide encoding for a tail of SEQ ID NO. 589, comprising a polypeptide being at least 70%, optionally at least about 80%, preferably at least about 85%, more preferably at least about 90% and most preferably at least about 95% homologous to amino in SEQ ID NO. 589.

According to preferred embodiments of the present invention, there is provided an isolated chimeric polypeptide encoding for SEQ ID NO. 589, comprising a first amino acid sequence being at least 90% homologous to amino acids 1-128 of SEQ ID NO. 638, which also corresponds to amino acids 1-128 of SEQ ID NO. 589, a bridging amino acid L corresponding to amino acid 129 of SEQ ID NO. 589, a second amino acid sequence being at least 90% homologous to amino acids 130-311 of SEQ ID NO. 638, which also corresponds to amino acids 130-311 of SEQ ID NO. 589, and a third amino acid sequence being at least 70%, optionally at least 80%, preferably at least 85%, more preferably at least 90% and most preferably at least 95% homologous to a polypeptide sequence corresponding to amino acids 312-315 of SEQ ID NO. 589, wherein said first amino acid sequence, bridging amino acid, second amino acid sequence and third amino acid sequence are contiguous and in a sequential order.

According to preferred embodiments of the present invention, there is provided an isolated polypeptide encoding for a tail of SEQ ID NO. 589, comprising a polypeptide being at least 70%, optionally at least about 80%, preferably at least about 85%, more preferably at least about 90% and most preferably at least about 95% homologous to amino in SEQ ID NO. 589.

According to preferred embodiments of the present invention, there is provided an isolated chimeric polypeptide encoding for SEQ ID NO. 589, comprising a first amino acid sequence being at least 90% homologous to amino acids 1-311 of Q9UJZ1 (SEQ ID NO:637), which also corresponds to amino acids 1-311 of SEQ ID NO. 589, and a second amino acid sequence being at least 70%, optionally at least 80%, preferably at least 85%, more preferably at least 90% and most preferably at least 95% homologous to a polypeptide sequence corresponding to amino acids 312-315 of SEQ ID NO. 589, wherein said first amino acid sequence and second amino acid sequence are contiguous and in a sequential order.

According to preferred embodiments of the present invention, there is provided an isolated polypeptide encoding for a tail of SEQ ID NO. 589, comprising a polypeptide being at least 70%, optionally at least about 80%, preferably at least about 85%, more preferably at least about 90% and most preferably at least about 95% homologous to amino in SEQ ID NO. 589.

According to preferred embodiments of the present invention, there is provided an isolated chimeric polypeptide encoding for SEQ ID NO. 590, comprising a first amino acid sequence being at least 70%, optionally at least 80%, preferably at least 85%, more preferably at least 90% and most preferably at least 95% homologous to a polypeptide sequence corresponding to amino acids 1-26 of SEQ ID NO. 590, a second amino acid sequence being at least 90% homologous to amino acids 13-187 of Q9P042 (SEQ ID NO:639), which also corresponds to amino acids 27-201 of SEQ ID NO. 590, a bridging amino acid A corresponding to amino acid 202 of SEQ ID NO. 590, a third amino acid sequence being at least 90% homologous to amino acids 189-254 of SEQ ID NO. 639, which also corresponds to amino acids 203-268 of SEQ ID NO. 590, and a fourth amino acid sequence being at least 70%, optionally at least 80%, preferably at least 85%, more preferably at least 90% and most preferably at least 95% homologous to a polypeptide sequence corresponding to amino acids 269-290 of SEQ ID NO. 590, wherein said first amino acid sequence, second amino acid sequence, bridging amino acid, third amino acid sequence and fourth amino acid sequence are contiguous and in a sequential order.

According to preferred embodiments of the present invention, there is provided an isolated polypeptide encoding for a head of SEQ ID NO. 590, comprising a polypeptide being at least 70%, optionally at least about 80%, preferably at least about 85%, more preferably at least about 90% and most preferably at least about 95% homologous to amino acids 1-26 of SEQ ID NO. 590.

According to preferred embodiments of the present invention, there is provided an isolated polypeptide encoding for a tail of SEQ ID NO. 590, comprising a polypeptide being at least 70%, optionally at least about 80%, preferably at least about 85%, more preferably at least about 90% and most preferably at least about 95% homologous to amino in SEQ ID NO. 590.

According to preferred embodiments of the present invention, there is provided an isolated chimeric polypeptide encoding for SEQ ID NO. 590, comprising a first amino acid sequence being at least 70%, optionally at least 80%, preferably at least 85%, more preferably at least 90% and most preferably at least 95% homologous to a polypeptide sequence corresponding to amino acids 1-109 of SEQ ID NO. 590, and a second amino acid sequence being at least 90% homologous to corresponding to amino acids 1-181 of SEQ ID NO. 640, which also corresponds to amino acids 110-290 of SEQ ID NO. 590, wherein said first amino acid sequence and second amino acid sequence are contiguous and in a sequential order.

According to preferred embodiments of the present invention, there is provided an isolated polypeptide encoding for a head of SEQ ID NO. 590, comprising a polypeptide being at least 70%, optionally at least about 80%, preferably at least about 85%, more preferably at least about 90% and most preferably at least about 95% homologous to amino acids 1-109 of SEQ ID NO. 590.

According to preferred embodiments of the present invention, there is provided an isolated chimeric polypeptide encoding for SEQ ID NO. 590, comprising a first amino acid sequence being at least 90% homologous to amino acids 1-128 of SEQ ID NO. 638, which also corresponds to amino acids 1-128 of SEQ ID NO. 590, a bridging amino acid L corresponding to amino acid 129 of SEQ ID NO. 590, a second amino acid sequence being at least 90% homologous to amino acids 130-268 of SEQ ID NO. 638, which also corresponds to amino acids 130-268 of SEQ ID NO. 590, and a third amino acid sequence being at least 70%, optionally at least 80%, preferably at least 85%, more preferably at least 90% and most preferably at least 95% homologous to a polypeptide sequence corresponding to amino acids 269-290 of SEQ ID NO. 590, wherein said first amino acid sequence, bridging amino acid, second amino acid sequence and third amino acid sequence are contiguous and in a sequential order.

According to preferred embodiments of the present invention, there is provided an isolated polypeptide encoding for a tail of SEQ ID NO. 590, comprising a polypeptide being at least 70%, optionally at least about 80%, preferably at least about 85%, more preferably at least about 90% and most preferably at least about 95% homologous to amino in SEQ ID NO. 590.

According to preferred embodiments of the present invention, there is provided an isolated chimeric polypeptide encoding for SEQ ID NO. 590, comprising a first amino acid sequence being at least 90% homologous to amino acids 1-268 of Q9UJZ1 (SEQ ID NO:637), which also corresponds to amino acids 1-268 of SEQ ID NO. 590, and a second amino acid sequence being at least 70%, optionally at least 80%, preferably at least 85%, more preferably at least 90% and most preferably at least 95% homologous to a polypeptide sequence corresponding to amino acids 269-290 of SEQ ID NO. 590, wherein said first amino acid sequence and second amino acid sequence are contiguous and in a sequential order.

According to preferred embodiments of the present invention, there is provided an isolated polypeptide encoding for a tail of SEQ ID NO. 590, comprising a polypeptide being at least 70%, optionally at least about 80%, preferably at least about 85%, more preferably at least about 90% and most preferably at least about 95% homologous to amino in SEQ ID NO. 590.

According to preferred embodiments of the present invention, there is provided an isolated chimeric polypeptide encoding for SEQ ID NO. 591, comprising a first amino acid sequence being at least 70%, optionally at least 80%, preferably at least 85%, more preferably at least 90% and most preferably at least 95% homologous to a polypeptide sequence corresponding to amino acids 1-26 of SEQ ID NO. 591, a second amino acid sequence being at least 90% homologous to amino acids 13-187 of SEQ ID NO. 639, which also corresponds to amino acids 27-201 of SEQ ID NO. 591, a bridging amino acid A corresponding to amino acid 202 of SEQ ID NO. 591, a third amino acid sequence being at least 90% homologous to amino acids 189-226 of SEQ ID NO. 639, which also corresponds to amino acids 282-397 of SEQ ID NO. 591, a fourth amino acid sequence being at least 70%, optionally at least 80%, preferably at least 85%, more preferably at least 90% and most preferably at least 95% homologous to a polypeptide sequence corresponding to amino acids 241-281 of SEQ ID NO. 591, and a fifth amino acid sequence being at least 90% homologous to amino acids 227-342 of SEQ ID NO. 639, which also corresponds to amino acids 282-397 of SEQ ID NO. 591, wherein said first amino acid sequence, second amino acid sequence, bridging amino acid, third amino acid sequence, fourth amino acid sequence and fifth amino acid sequence are contiguous and in a sequential order.

According to preferred embodiments of the present invention, there is provided an isolated polypeptide encoding for a head of SEQ ID NO. 591, comprising a polypeptide being at least 70%, optionally at least about 80%, preferably at least about 85%, more preferably at least about 90% and most preferably at least about 95% homologous to amino acids 1-26 of SEQ ID NO. 591.

According to preferred embodiments of the present invention, there is provided an isolated polypeptide encoding for an edge portion of SEQ ID NO. 591, comprising an amino acid sequence being at least 70%, optionally at least about 80%, preferably at least about 85%, more preferably at least about 90% and most preferably at least about 95% homologous to the sequence encoding amino corresponding to SEQ ID NO. 591.

According to preferred embodiments of the present invention, there is provided an isolated chimeric polypeptide encoding for SEQ ID NO. 591, comprising a first amino acid sequence being at least 70%, optionally at least 80%, preferably at least 85%, more preferably at least 90% and most preferably at least 95% homologous to a polypeptide sequence corresponding to amino acids 1-109 of SEQ ID NO. 591, a second amino acid sequence being at least 90% homologous to amino acids 1-131 of SEQ ID NO. 640, which also corresponds to amino acids 110-240 of SEQ ID NO. 591, a third amino acid sequence being at least 70%, optionally at least 80%, preferably at least 85%, more preferably at least 90% and most preferably at least 95% homologous to a polypeptide sequence corresponding to amino acids 241-281 of SEQ ID NO. 591, a fourth amino acid sequence being at least 90% homologous to amino acids 132-159 of SEQ ID NO. 640, which also corresponds to amino acids 282-309 of SEQ ID NO. 591, and a fifth amino acid sequence being at least 70%, optionally at least 80%, preferably at least 85%, more preferably at least 90% and most preferably at least 95% homologous to a polypeptide sequence corresponding to amino acids 310-397 of SEQ ID NO. 591, wherein said first amino acid sequence, second amino acid sequence, third amino acid sequence, fourth amino acid sequence and fifth amino acid sequence are contiguous and in a sequential order.

According to preferred embodiments of the present invention, there is provided an isolated polypeptide encoding for a head of SEQ ID NO. 591, comprising a polypeptide being at least 70%, optionally at least about 80%, preferably at least about 85%, more preferably at least about 90% and most preferably at least about 95% homologous to amino acids 1-109 of SEQ ID NO. 591.

According to preferred embodiments of the present invention, there is provided an isolated polypeptide encoding for an edge portion of SEQ ID NO. 591, comprising an amino acid sequence being at least 70%, optionally at least about 80%, preferably at least about 85%, more preferably at least about 90% and most preferably at least about 95% homologous to the sequence encoding for amino corresponding to SEQ ID NO. 591.

According to preferred embodiments of the present invention, there is provided an isolated polypeptide encoding for a tail of SEQ ID NO. 591, comprising a polypeptide being at least 70%, optionally at least about 80%, preferably at least about 85%, more preferably at least about 90% and most preferably at least about 95% homologous to amino in SEQ ID NO. 591.

According to preferred embodiments of the present invention, there is provided an isolated chimeric polypeptide encoding for SEQ ID NO. 591, comprising a first amino acid sequence being at least 90% homologous to amino acids 1-128 of Q96FY2 (SEQ ID NO:638), which also corresponds to amino acids 1-128 of SEQ ID NO. 591, a bridging amino acid L corresponding to amino acid 129 of SEQ ID NO. 591, a second amino acid sequence being at least 90% homologous to amino acids 241-281 of SEQ ID NO. 638, which also corresponds to amino acids 130-240 of SEQ ID NO. 591, a third amino acid sequence being at least 70%, optionally at least 80%, preferably at least 85%, more preferably at least 90% and most preferably at least 95% homologous to a polypeptide sequence corresponding to amino acids 241-281 of SEQ ID NO. 591, and a fourth amino acid sequence being at least 90% homologous to amino acids 241-356 of SEQ ID NO. 638, which also corresponds to amino acids 282-397 of SEQ ID NO. 591, wherein said first amino acid sequence, bridging amino acid, second amino acid sequence, third amino acid sequence and fourth amino acid sequence are contiguous and in a sequential order.

According to preferred embodiments of the present invention, there is provided an isolated polypeptide encoding for an edge portion of SEQ ID NO. 591, comprising an amino acid sequence being at least 70%, optionally at least about 80%, preferably at least about 85%, more preferably at least about 90% and most preferably at least about 95% homologous to the sequence encoding for amino corresponding to SEQ ID NO. 591.

According to preferred embodiments of the present invention, there is provided an isolated chimeric polypeptide encoding for SEQ ID NO. 591, comprising a first amino acid sequence being at least 90% homologous to amino acids 1-240 of Q9UJZ1 (SEQ ID NO:637), which also corresponds to amino acids 1-240 of SEQ ID NO. 591, a second amino acid sequence being at least 70%, optionally at least 80%, preferably at least 85%, more preferably at least 90% and most preferably at least 95% homologous to a polypeptide sequence corresponding to amino acids 241-281 of SEQ ID NO. 591, and a third amino acid sequence being at least 90% homologous to amino acids 241-356 of Q9UJZ1 (SEQ ID NO:637), which also corresponds to amino acids 282-397 of SEQ ID NO. 591, wherein said first amino acid sequence, second amino acid sequence and third amino acid sequence are contiguous and in a sequential order.

According to preferred embodiments of the present invention, there is provided an isolated polypeptide encoding for an edge portion of SEQ ID NO. 591, comprising an amino acid sequence being at least 70%, optionally at least about 80%, preferably at least about 85%, more preferably at least about 90% and most preferably at least about 95% homologous to the sequence encoding for amino corresponding to SEQ ID NO. 591.

According to preferred embodiments of the present invention, there is provided an isolated chimeric polypeptide encoding for SEQ ID NO. 592, comprising a first amino acid sequence being at least 70%, optionally at least 80%, preferably at least 85%, more preferably at least 90% and most preferably at least 95% homologous to a polypeptide sequence corresponding to amino acids 1-26 of SEQ ID NO. 592, a second amino acid sequence being at least 90% homologous to amino acids 13-187 of SEQ ID NO. 639, which also corresponds to amino acids 27-201 of SEQ ID NO. 592, a bridging amino acid A corresponding to amino acid 202 of SEQ ID NO. 592, a third amino acid sequence being at least 90% homologous to amino acids 189-254 of SEQ ID NO. 639, which also corresponds to amino acids 203-268 of SEQ ID NO. 592, and a fourth amino acid sequence being at least 90% homologous to amino acids 298-342 of SEQ ID NO. 639, which also corresponds to amino acids 269-313 of SEQ ID NO. 592, wherein said first amino acid sequence, second amino acid sequence, bridging amino acid, third amino acid sequence and fourth amino acid sequence are contiguous and in a sequential order.

According to preferred embodiments of the present invention, there is provided an isolated polypeptide encoding for a head of SEQ ID NO. 592, comprising a polypeptide being at least 70%, optionally at least about 80%, preferably at least about 85%, more preferably at least about 90% and most preferably at least about 95% homologous to amino acids 1-26 of SEQ ID NO. 592.

According to preferred embodiments of the present invention, there is provided an isolated chimeric polypeptide encoding for an edge portion of SEQ ID NO. 592, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise HA, having a structure as follows: a sequence starting from any of amino acid numbers 268−x to 268; and ending at any of amino acid numbers 269+((n−2)−x), in which x varies from 0 to n−2.

According to preferred embodiments of the present invention, there is provided an isolated chimeric polypeptide encoding for SEQ ID NO. 592, comprising a first amino acid sequence being at least 70%, optionally at least 80%, preferably at least 85%, more preferably at least 90% and most preferably at least 95% homologous to a polypeptide sequence amino acids 1-109 of SEQ ID NO. 592, a second amino acid sequence being at least 90% homologous to amino acids 1-159 of SEQ ID NO. 640, which also corresponds to amino acids 110-268 of SEQ ID NO. 592, and a third amino acid sequence being at least 70%, optionally at least 80%, preferably at least 85%, more preferably at least 90% and most preferably at least 95% homologous to a polypeptide sequence corresponding to amino acids 269-313 of SEQ ID NO. 592, wherein said first amino acid sequence, second amino acid sequence and third amino acid sequence are contiguous and in a sequential order.

According to preferred embodiments of the present invention, there is provided an isolated polypeptide encoding for a head of SEQ ID NO. 592, comprising a polypeptide being at least 70%, optionally at least about 80%, preferably at least about 85%, more preferably at least about 90% and most preferably at least about 95% homologous to amino acids 1-109 of SEQ ID NO. 592.

According to preferred embodiments of the present invention, there is provided an isolated polypeptide encoding for a tail of SEQ ID NO. 592, comprising a polypeptide being at least 70%, optionally at least about 80%, preferably at least about 85%, more preferably at least about 90% and most preferably at least about 95% homologous to amino in SEQ ID NO. 592.

According to preferred embodiments of the present invention, there is provided an isolated chimeric polypeptide encoding for SEQ ID NO. 592, comprising a first amino acid sequence being at least 90% homologous to amino acids 1-128 of SEQ ID NO. 638, which also corresponds to amino acids 1-128 of SEQ ID NO. 592, a bridging amino acid L corresponding to amino acid 129 of SEQ ID NO. 592, a second amino acid sequence being at least 90% homologous to amino acids 130-268 of SEQ ID NO. 638, which also corresponds to amino acids 130-268 of SEQ ID NO. 592, and a third amino acid sequence being at least 90% homologous to amino acids 312-356 of SEQ ID NO. 638, which also corresponds to amino acids 269-313 of SEQ ID NO. 592, wherein said first amino acid sequence, bridging amino acid, second amino acid sequence and third amino acid sequence are contiguous and in a sequential order.

According to preferred embodiments of the present invention, there is provided an isolated chimeric polypeptide encoding for an edge portion of SEQ ID NO. 592, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise HA, having a structure as follows: a sequence starting from any of amino acid numbers 268−x to 268; and ending at any of amino acid numbers 269+((n−2)−x), in which x varies from 0 to n−2.

According to preferred embodiments of the present invention, there is provided an isolated chimeric polypeptide encoding for SEQ ID NO. 592, comprising a first amino acid sequence being at least 90% homologous to amino acids 1-268 of Q9UJZ1 (SEQ ID NO:637), which also corresponds to amino acids 1-268 of SEQ ID NO. 592, and a second amino acid sequence being at least 90% homologous to amino acids 312-356 of Q9UJZ1 (SEQ ID NO:637), which also corresponds to amino acids 269-313 of SEQ ID NO. 592, wherein said first amino acid sequence and second amino acid sequence are contiguous and in a sequential order.

According to preferred embodiments of the present invention, there is provided an isolated chimeric polypeptide encoding for an edge portion of SEQ ID NO. 592, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise HA, having a structure as follows: a sequence starting from any of amino acid numbers 268−x to 268; and ending at any of amino acid numbers 269+((n−2)−x), in which x varies from 0 to n−2.

According to preferred embodiments of the present invention, there is provided an isolated chimeric polypeptide encoding for SEQ ID NO. 592, comprising a first amino acid sequence being at least 70%, optionally at least 80%, preferably at least 85%, more preferably at least 90% and most preferably at least 95% homologous to a polypeptide sequence amino acids 1-109 of SEQ ID NO. 592, a second amino acid sequence being at least 90% homologous to amino acids 1-159 of SEQ ID NO. 640, which also corresponds to amino acids 110-268 of SEQ ID NO. 592, and a third amino acid sequence being at least 70%, optionally at least 80%, preferably at least 85%, more preferably at least 90% and most preferably at least 95% homologous to a polypeptide sequence corresponding to amino acids 269-313 of SEQ ID NO. 592, wherein said first amino acid sequence, second amino acid sequence and third amino acid sequence are contiguous and in a sequential order.

According to preferred embodiments of the present invention, there is provided an isolated polypeptide encoding for a head of SEQ ID NO. 592, comprising a polypeptide being at least 70%, optionally at least about 80%, preferably at least about 85%, more preferably at least about 90% and most preferably at least about 95% homologous to amino acids 269-313 of SEQ ID NO. 592.

According to preferred embodiments of the present invention, there is provided an isolated polypeptide encoding for a tail of SEQ ID NO. 592, comprising a polypeptide being at least 70%, optionally at least about 80%, preferably at least about 85%, more preferably at least about 90% and most preferably at least about 95% homologous to amino in SEQ ID NO. 592.

According to preferred embodiments of the present invention, there is provided an isolated chimeric polypeptide encoding for SEQ ID NO. 592, comprising a first amino acid sequence being at least 90% homologous to amino acids 1-128 of SEQ ID NO. 638, which also corresponds to amino acids 1-128 of SEQ ID NO. 592, a bridging amino acid L corresponding to amino acid 129 of SEQ ID NO. 592, a second amino acid sequence being at least 90% homologous to amino acids 130-268 of SEQ ID NO. 638, which also corresponds to amino acids 130-268 of SEQ ID NO. 592, and a third amino acid sequence being at least 90% homologous to amino acids 312-356 of SEQ ID NO. 638, which also corresponds to amino acids 269-313 of SEQ ID NO. 592, wherein said first amino acid sequence, bridging amino acid, second amino acid sequence and third amino acid sequence are contiguous and in a sequential order.

According to preferred embodiments of the present invention, there is provided an isolated chimeric polypeptide encoding for an edge portion of SEQ ID NO. 592, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise HA, having a structure as follows: a sequence starting from any of amino acid numbers 268−x to 268; and ending at any of amino acid numbers 269+((n−2)−x), in which x varies from 0 to n−2.

According to preferred embodiments of the present invention, there is provided an isolated chimeric polypeptide encoding for SEQ ID NO. 592, comprising a first amino acid sequence being at least 90% homologous to amino acids 1-268 of Q9UJZ1 (SEQ ID NO:637), which also corresponds to amino acids 1-268 of SEQ ID NO. 592, and a second amino acid sequence being at least 90% homologous to amino acids 312-356 of Q9UJZ1 (SEQ ID NO:637), which also corresponds to amino acids 269-313 of SEQ ID NO. 592, wherein said first amino acid sequence and second amino acid sequence are contiguous and in a sequential order.

According to preferred embodiments of the present invention, there is provided an isolated chimeric polypeptide encoding for an edge portion of SEQ ID NO. 592, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise HA, having a structure as follows: a sequence starting from any of amino acid numbers 268−x to 268; and ending at any of amino acid numbers 269+((n−2)−x), in which x varies from 0 to n−2.

According to preferred embodiments of the present invention, there is provided an isolated chimeric polypeptide encoding for SEQ ID NO. 593, comprising a first amino acid sequence being at least 70%, optionally at least 80%, preferably at least 85%, more preferably at least 90% and most preferably at least 95% homologous to a polypeptide sequence corresponding to amino acids 1-26 of SEQ ID NO. 593, a second amino acid sequence being at least 90% homologous to amino acids 13-187 of SEQ ID NO. 639, which also corresponds to amino acids 27-201 of SEQ ID NO. 593, a bridging amino acid A corresponding to amino acid 202 of SEQ ID NO. 593, a third amino acid sequence being at least 90% homologous to amino acids 227-254 of SEQ ID NO. 639, which also corresponds to amino acids 282-309 of SEQ ID NO. 593, a fourth amino acid sequence being at least 70%, optionally at least 80%, preferably at least 85%, more preferably at least 90% and most preferably at least 95% homologous to a polypeptide sequence corresponding to amino acids 241-281 of SEQ ID NO. 593, a fifth amino acid sequence being at least 90% homologous to amino acids 227-254 of SEQ ID NO. 639, which also corresponds to amino acids 282-309 of SEQ ID NO. 593, and a sixth amino acid sequence being at least 70%, optionally at least 80%, preferably at least 85%, more preferably at least 90% and most preferably at least 95% homologous to a polypeptide sequence corresponding to amino acids 310-331 of SEQ ID NO. 593, wherein said first amino acid sequence, second amino acid sequence, bridging amino acid, third amino acid sequence, fourth amino acid sequence, fifth amino acid sequence and sixth amino acid sequence are contiguous and in a sequential order.

According to preferred embodiments of the present invention, there is provided an isolated polypeptide encoding for a head of SEQ ID NO. 593, comprising a polypeptide being at least 70%, optionally at least about 80%, preferably at least about 85%, more preferably at least about 90% and most preferably at least about 95% homologous to amino acids 1-26 of SEQ ID NO. 593.

According to preferred embodiments of the present invention, there is provided an isolated polypeptide encoding for an edge portion of SEQ ID NO. 593, comprising an amino acid sequence being at least 70%, optionally at least about 80%, preferably at least about 85%, more preferably at least about 90% and most preferably at least about 95% homologous to the sequence encoding for amino corresponding to SEQ ID NO. 593.

According to preferred embodiments of the present invention, there is provided an isolated polypeptide encoding for a tail of SEQ ID NO. 593, comprising a polypeptide being at least 70%, optionally at least about 80%, preferably at least about 85%, more preferably at least about 90% and most preferably at least about 95% homologous to amino in SEQ ID NO. 593.

According to preferred embodiments of the present invention, there is provided an isolated chimeric polypeptide encoding for SEQ ID NO. 593, comprising a first amino acid sequence being at least 70%, optionally at least 80%, preferably at least 85%, more preferably at least 90% and most preferably at least 95% homologous to a polypeptide sequence corresponding to amino acids 1-109 of SEQ ID NO. 593, a second amino acid sequence being at least 90% homologous to amino acids 1-131 of SEQ ID NO. 640, which also corresponds to amino acids 110-240 of SEQ ID NO. 593, a third amino acid sequence being at least 70%, optionally at least 80%, preferably at least 85%, more preferably at least 90% and most preferably at least 95% homologous to a polypeptide sequence corresponding to amino acids 241-281 of SEQ ID NO. 593, and a fourth amino acid sequence being at least 90% homologous to amino acids 132-181 of SEQ ID NO. 640, which also corresponds to amino acids 282-331 of SEQ ID NO. 593, wherein said first amino acid sequence, second amino acid sequence, third amino acid sequence and fourth amino acid sequence are contiguous and in a sequential order.

According to preferred embodiments of the present invention, there is provided an isolated polypeptide encoding for a head of SEQ ID NO. 593, comprising a polypeptide being at least 70%, optionally at least about 80%, preferably at least about 85%, more preferably at least about 90% and most preferably at least about 95% homologous to amino acids 1-109 of SEQ ID NO. 593.

According to preferred embodiments of the present invention, there is provided an isolated polypeptide encoding for an edge portion of SEQ ID NO. 593, comprising an amino acid sequence being at least 70%, optionally at least about 80%, preferably at least about 85%, more preferably at least about 90% and most preferably at least about 95% homologous to the sequence encoding for amino corresponding to SEQ ID NO. 593.

According to preferred embodiments of the present invention, there is provided an isolated chimeric polypeptide encoding for SEQ ID NO. 593, comprising a first amino acid sequence being at least 90% homologous to amino acids 1-128 of SEQ ID NO. 638, which also corresponds to amino acids 1-128 of SEQ ID NO. 593, a bridging amino acid L corresponding to amino acid 129 of SEQ ID NO. 593, a second amino acid sequence being at least 90% homologous to amino acids 130-240 of SEQ ID NO. 638, which also corresponds to amino acids 130-240 of SEQ ID NO. 593, a third amino acid sequence being at least 70%, optionally at least 80%, preferably at least 85%, more preferably at least 90% and most preferably at least 95% homologous to a polypeptide sequence corresponding to amino acids 241-281 of SEQ ID NO. 593, a fourth amino acid sequence being at least 90% homologous to amino acids 241-268 of SEQ ID NO. 638, which also corresponds to amino acids 282-309 of SEQ ID NO. 593, and a fifth amino acid sequence being at least 70%, optionally at least 80%, preferably at least 85%, more preferably at least 90% and most preferably at least 95% homologous to a polypeptide sequence corresponding to amino acids 310-331 of SEQ ID NO. 593, wherein said first amino acid sequence, bridging amino acid, second amino acid sequence, third amino acid sequence, fourth amino acid sequence and fifth amino acid sequence are contiguous and in a sequential order.

According to preferred embodiments of the present invention, there is provided an isolated polypeptide encoding for an edge portion of SEQ ID NO. 593, comprising an amino acid sequence being at least 70%, optionally at least about 80%, preferably at least about 85%, more preferably at least about 90% and most preferably at least about 95% homologous to the sequence encoding for amino corresponding to SEQ ID NO. 593.

According to preferred embodiments of the present invention, there is provided an isolated polypeptide encoding for a tail of SEQ ID NO. 593, comprising a polypeptide being at least 70%, optionally at least about 80%, preferably at least about 85%, more preferably at least about 90% and most preferably at least about 95% homologous to amino in SEQ ID NO. 593.

According to preferred embodiments of the present invention, there is provided an isolated chimeric polypeptide encoding for SEQ ID NO. 593, comprising a first amino acid sequence being at least 90% homologous to amino acids 1-240 of Q9UJZ1 (SEQ ID NO:637), which also corresponds to amino acids 1-240 of SEQ ID NO. 593, a second amino acid sequence being at least 70%, optionally at least 80%, preferably at least 85%, more preferably at least 90% and most preferably at least 95% homologous to a polypeptide sequence corresponding to amino acids 310-331 of SEQ ID NO. 593, a third amino acid sequence being at least 90% homologous to amino acids 241-268 of Q9UJZ1 (SEQ ID NO:637), which also corresponds to amino acids 282-309 of SEQ ID NO. 593, and a fourth amino acid sequence being at least 70%, optionally at least 80%, preferably at least 85%, more preferably at least 90% and most preferably at least 95% homologous to a polypeptide sequence corresponding to amino acids 310-331 of SEQ ID NO. 593, wherein said first amino acid sequence, second amino acid sequence, third amino acid sequence and fourth amino acid sequence are contiguous and in a sequential order.

According to preferred embodiments of the present invention, there is provided an isolated polypeptide encoding for an edge portion of SEQ ID NO. 593, comprising an amino acid sequence being at least 70%, optionally at least about 80%, preferably at least about 85%, more preferably at least about 90% and most preferably at least about 95% homologous to the sequence encoding for amino corresponding to SEQ ID NO. 593.

According to preferred embodiments of the present invention, there is provided an isolated polypeptide encoding for a tail of SEQ ID NO. 593, comprising a polypeptide being at least 70%, optionally at least about 80%, preferably at least about 85%, more preferably at least about 90% and most preferably at least about 95% homologous to amino in SEQ ID NO. 593.

According to preferred embodiments of the present invention, there is provided an isolated chimeric polypeptide encoding for SEQ ID NO. 594, comprising a first amino acid sequence being at least 70%, optionally at least 80%, preferably at least 85%, more preferably at least 90% and most preferably at least 95% homologous to a polypeptide sequence corresponding to amino acids 1-26 of SEQ ID NO. 594, a second amino acid sequence being at least 90% homologous to amino acids 13-134 of SEQ ID NO. 639, which also corresponds to amino acids 27-148 of SEQ ID NO. 594, and a third amino acid sequence being at least 70%, optionally at least 80%, preferably at least 85%, more preferably at least 90% and most preferably at least 95% homologous to a polypeptide sequence corresponding to amino acids 149-183 of SEQ ID NO. 594, wherein said first amino acid sequence, second amino acid sequence and third amino acid sequence are contiguous and in a sequential order.

According to preferred embodiments of the present invention, there is provided an isolated polypeptide encoding for a head of SEQ ID NO. 594, comprising a polypeptide being at least 70%, optionally at least about 80%, preferably at least about 85%, more preferably at least about 90% and most preferably at least about 95% homologous to amino acids 1-26 of SEQ ID NO. 594.

According to preferred embodiments of the present invention, there is provided an isolated polypeptide encoding for a tail of SEQ ID NO. 594, comprising a polypeptide being at least 70%, optionally at least about 80%, preferably at least about 85%, more preferably at least about 90% and most preferably at least about 95% homologous to amino in SEQ ID NO. 594.

According to preferred embodiments of the present invention, there is provided an isolated chimeric polypeptide encoding for SEQ ID NO. 594, comprising a first amino acid sequence being at least 90% homologous to amino acids 1-128 of SEQ ID NO. 638, which also corresponds to amino acids 1-128 of SEQ ID NO. 594, a bridging amino acid L corresponding to amino acid 129 of SEQ ID NO. 594, a second amino acid sequence being at least 90% homologous to amino acids 130-148 of SEQ ID NO. 638, which also corresponds to amino acids 130-148 of SEQ ID NO. 594, and a third amino acid sequence being at least 70%, optionally at least 80%, preferably at least 85%, more preferably at least 90% and most preferably at least 95% homologous to a polypeptide sequence corresponding to amino acids 149-183 of SEQ ID NO. 594, wherein said first amino acid sequence, bridging amino acid, second amino acid sequence and third amino acid sequence are contiguous and in a sequential order.

According to preferred embodiments of the present invention, there is provided an isolated polypeptide encoding for a tail of SEQ ID NO. 594, comprising a polypeptide being at least 70%, optionally at least about 80%, preferably at least about 85%, more preferably at least about 90% and most preferably at least about 95% homologous to amino in SEQ ID NO. 594.

According to preferred embodiments of the present invention, there is provided an isolated chimeric polypeptide encoding for SEQ ID NO. 594, comprising a first amino acid sequence being at least 90% homologous to amino acids 1-148 of Q9UJZ1 (SEQ ID NO:637), which also corresponds to amino acids 1-148 of SEQ ID NO. 594, and a second amino acid sequence being at least 70%, optionally at least 80%, preferably at least 85%, more preferably at least 90% and most preferably at least 95% homologous to a polypeptide sequence corresponding to amino acids 149-183 of SEQ ID NO. 594, wherein said first amino acid sequence and second amino acid sequence are contiguous and in a sequential order.

According to preferred embodiments of the present invention, there is provided an isolated polypeptide encoding for a tail of SEQ ID NO. 594, comprising a polypeptide being at least 70%, optionally at least about 80%, preferably at least about 85%, more preferably at least about 90% and most preferably at least about 95% homologous to amino in SEQ ID NO. 594.

According to preferred embodiments of the present invention, there is provided an isolated chimeric polypeptide encoding for SEQ ID NO. 595, comprising a first amino acid sequence being at least 70%, optionally at least 80%, preferably at least 85%, more preferably at least 90% and most preferably at least 95% homologous to a polypeptide sequence corresponding to amino acids 1-26 of SEQ ID NO. 595, a second amino acid sequence being at least 90% homologous to amino acids 13-180 of SEQ ID NO. 639, which also corresponds to amino acids 27-194 of SEQ ID NO. 595, and a third amino acid sequence being at least 70%, optionally at least 80%, preferably at least 85%, more preferably at least 90% and most preferably at least 95% homologous to a polypeptide sequence corresponding to amino acids 195-220 of SEQ ID NO. 595, wherein said first amino acid sequence, second amino acid sequence and third amino acid sequence are contiguous and in a sequential order.

According to preferred embodiments of the present invention, there is provided an isolated polypeptide encoding for a head of SEQ ID NO. 595, comprising a polypeptide being at least 70%, optionally at least about 80%, preferably at least about 85%, more preferably at least about 90% and most preferably at least about 95% homologous to amino acids 1-26 of SEQ ID NO. 595.

According to preferred embodiments of the present invention, there is provided an isolated polypeptide encoding for a tail of SEQ ID NO. 595, comprising a polypeptide being at least 70%, optionally at least about 80%, preferably at least about 85%, more preferably at least about 90% and most preferably at least about 95% homologous to amino in SEQ ID NO. 595.

According to preferred embodiments of the present invention, there is provided an isolated chimeric polypeptide encoding for SEQ ID NO. 595, comprising a first amino acid sequence being at least 90% homologous to amino acids 1-128 of SEQ ID NO. 638, which also corresponds to amino acids 1-128 of SEQ ID NO. 595, a bridging amino acid L corresponding to amino acid 129 of SEQ ID NO. 595, a second amino acid sequence being at least 90% homologous to amino acids 130-194 of SEQ ID NO. 638, which also corresponds to amino acids 130-194 of SEQ ID NO. 595, and a third amino acid sequence being at least 70%, optionally at least 80%, preferably at least 85%, more preferably at least 90% and most preferably at least 95% homologous to a polypeptide sequence corresponding to amino acids 195-220 of SEQ ID NO. 595, wherein said first amino acid sequence, bridging amino acid, second amino acid sequence and third amino acid sequence are contiguous and in a sequential order.

According to preferred embodiments of the present invention, there is provided an isolated polypeptide encoding for a tail of SEQ ID NO. 595, comprising a polypeptide being at least 70%, optionally at least about 80%, preferably at least about 85%, more preferably at least about 90% and most preferably at least about 95% homologous to amino in SEQ ID NO. 595.

According to preferred embodiments of the present invention, there is provided an isolated chimeric polypeptide encoding for SEQ ID NO. 595, comprising a first amino acid sequence being at least 90% homologous to amino acids 1-194 of Q9UJZ1 (SEQ ID NO:637), which also corresponds to amino acids 1-194 of SEQ ID NO. 595, and a second amino acid sequence being at least 70%, optionally at least 80%, preferably at least 85%, more preferably at least 90% and most preferably at least 95% homologous to a polypeptide sequence corresponding to amino acids 195-220 of SEQ ID NO. 595, wherein said first amino acid sequence and second amino acid sequence are contiguous and in a sequential order.

According to preferred embodiments of the present invention, there is provided an isolated polypeptide encoding for a tail of SEQ ID NO. 595, comprising a polypeptide being at least 70%, optionally at least about 80%, preferably at least about 85%, more preferably at least about 90% and most preferably at least about 95% homologous to amino in SEQ ID NO. 595.

According to preferred embodiments of the present invention, there is provided an isolated chimeric polypeptide encoding for SEQ ID NO. 596, comprising a first amino acid sequence being at least 70%, optionally at least 80%, preferably at least 85%, more preferably at least 90% and most preferably at least 95% homologous to a polypeptide sequence corresponding to amino acids 1-26 of SEQ ID NO. 596, a second amino acid sequence being at least 90% homologous to amino acids 13-134 of SEQ ID NO. 639, which also corresponds to amino acids 27-148 of SEQ ID NO. 596, a third amino acid sequence being at least 90% homologous to amino acids 180-187 of SEQ ID NO. 639, which also corresponds to amino acids 149-156 of SEQ ID NO. 596, a bridging amino acid A corresponding to amino acid 157 of SEQ ID NO. 596, and a fourth amino acid sequence being at least 90% homologous to amino acids 189-342 of SEQ ID NO. 639, which also corresponds to amino acids 158-311 of SEQ ID NO. 596, wherein said first amino acid sequence, second amino acid sequence, third amino acid sequence, bridging amino acid and fourth amino acid sequence are contiguous and in a sequential order.

According to preferred embodiments of the present invention, there is provided an isolated polypeptide encoding for a head of SEQ ID NO. 596, comprising a polypeptide being at least 70%, optionally at least about 80%, preferably at least about 85%, more preferably at least about 90% and most preferably at least about 95% homologous to amino acids 1-26 of SEQ ID NO. 596.

According to preferred embodiments of the present invention, there is provided an isolated chimeric polypeptide encoding for an edge portion of SEQ ID NO. 596, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RV, having a structure as follows: a sequence starting from any of amino acid numbers 148−x to 148; and ending at any of amino acid numbers 149+((n−2)−x), in which x varies from 0 to n−2.

According to preferred embodiments of the present invention, there is provided an isolated chimeric polypeptide encoding for SEQ ID NO. 596, comprising a first amino acid sequence being at least 70%, optionally at least 80%, preferably at least 85%, more preferably at least 90% and most preferably at least 95% homologous to a polypeptide sequence corresponding to amino acids 149-223 of SEQ ID NO. 596, a second amino acid sequence being at least 90% homologous to amino acids 1-39 of SEQ ID NO. 640, which also corresponds to amino acids 110-148 of SEQ ID NO. 596, a third amino acid sequence being at least 90% homologous to amino acids 85-159 of SEQ ID NO. 640, which also corresponds to amino acids 149-223 of SEQ ID NO. 596, and a fourth amino acid sequence being at least 70%, optionally at least 80%, preferably at least 85%, more preferably at least 90% and most preferably at least 95% homologous to a polypeptide sequence corresponding to amino acids 224-311 of SEQ ID NO. 596, wherein said first amino acid sequence, second amino acid sequence, third amino acid sequence and fourth amino acid sequence are contiguous and in a sequential order.

According to preferred embodiments of the present invention, there is provided an isolated polypeptide encoding for a head of SEQ ID NO. 596, comprising a polypeptide being at least 70%, optionally at least about 80%, preferably at least about 85%, more preferably at least about 90% and most preferably at least about 95% homologous to amino acids 1-109 of SEQ ID NO. 596.

According to preferred embodiments of the present invention, there is provided an isolated chimeric polypeptide encoding for an edge portion of SEQ ID NO. 596, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RV, having a structure as follows: a sequence starting from any of amino acid numbers 148−x to 148; and ending at any of amino acid numbers 149+((n−2)−x), in which x varies from 0 to n−2.

According to preferred embodiments of the present invention, there is provided an isolated polypeptide encoding for a tail of SEQ ID NO. 596, comprising a polypeptide being at least 70%, optionally at least about 80%, preferably at least about 85%, more preferably at least about 90% and most preferably at least about 95% homologous to amino in SEQ ID NO. 596.

According to preferred embodiments of the present invention, there is provided an isolated chimeric polypeptide encoding for SEQ ID NO. 596, comprising a first amino acid sequence being at least 90% homologous to amino acids 1-128 of Q96FY2 (SEQ ID NO:638), which also corresponds to amino acids 1-128 of SEQ ID NO. 596, a bridging amino acid L corresponding to amino acid 129 of SEQ ID NO. 596, a second amino acid sequence being at least 90% homologous to amino acids 130-148 of SEQ ID NO. 638, which also corresponds to amino acids 130-148 of SEQ ID NO. 596, and a third amino acid sequence being at least 90% homologous to corresponding to amino acids 194-356 of SEQ ID NO. 638, which also corresponds to amino acids 149-311 of SEQ ID NO. 596, wherein said first amino acid sequence, bridging amino acid, second amino acid sequence and third amino acid sequence are contiguous and in a sequential order.

According to preferred embodiments of the present invention, there is provided an isolated chimeric polypeptide encoding for an edge portion of SEQ ID NO. 596, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RV, having a structure as follows: a sequence starting from any of amino acid numbers 148−x to 148; and ending at any of amino acid numbers 149+((n−2)−x), in which x varies from 0 to n−2.

According to preferred embodiments of the present invention, there is provided an isolated chimeric polypeptide encoding for SEQ ID NO. 596, comprising a first amino acid sequence being at least 90% homologous to amino acids 1-148 of Q9UJZ1 (SEQ ID NO:637), which also corresponds to amino acids 1-148 of SEQ ID NO. 596, and a second amino acid sequence being at least 90% homologous to amino acids 194-356 of Q9UJZ1 (SEQ ID NO:637), which also corresponds to amino acids 149-311 of SEQ ID NO. 596, wherein said first amino acid sequence and second amino acid sequence are contiguous and in a sequential order.

According to preferred embodiments of the present invention, there is provided an isolated chimeric polypeptide encoding for an edge portion of SEQ ID NO. 596, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RV, having a structure as follows: a sequence starting from any of amino acid numbers 148−x to 148; and ending at any of amino acid numbers 149+((n−2)−x), in which x varies from 0 to n−2.

According to preferred embodiments of the present invention, there is provided an isolated chimeric polypeptide encoding for SEQ ID NO. 597, comprising a first amino acid sequence being at least 70%, optionally at least 80%, preferably at least 85%, more preferably at least 90% and most preferably at least 95% homologous to a polypeptide sequence corresponding to amino acids 1-26 of SEQ ID NO. 597, a second amino acid sequence being at least 90% homologous to amino acids 13-143 of SEQ ID NO. 639, which also corresponds to amino acids 27-157 of SEQ ID NO. 597, and a third amino acid sequence being at least 90% homologous to amino acids 295-342 of SEQ ID NO. 639, which also corresponds to amino acids 158-205 of SEQ ID NO. 597, wherein said first amino acid sequence, second amino acid sequence and third amino acid sequence are contiguous and in a sequential order.

According to preferred embodiments of the present invention, there is provided an isolated polypeptide encoding for a head of SEQ ID NO. 597, comprising a polypeptide being at least 70%, optionally at least about 80%, preferably at least about 85%, more preferably at least about 90% and most preferably at least about 95% homologous to amino acids 1-26 of SEQ ID NO. 597.

According to preferred embodiments of the present invention, there is provided an isolated chimeric polypeptide encoding for an edge portion of SEQ ID NO. 597, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IV, having a structure as follows: a sequence starting from any of amino acid numbers 157−x to 157; and ending at any of amino acid numbers 158+((n−2)−x), in which x varies from 0 to n−2.

According to preferred embodiments of the present invention, there is provided an isolated chimeric polypeptide encoding for SEQ ID NO. 597, comprising a first amino acid sequence being at least 90% homologous to amino acids 1-128 of Q96FY2 (SEQ ID NO:638), which also corresponds to amino acids 1-128 of SEQ ID NO. 597, a bridging amino acid L corresponding to amino acid 129 of SEQ ID NO. 597, a second amino acid sequence being at least 90% homologous to amino acids 130-157 of SEQ ID NO. 639, which also corresponds to amino acids 130-157 of SEQ ID NO. 597, and a third amino acid sequence being at least 90% homologous to amino acids 309-356 of ID NO. 639, which also corresponds to amino acids 158-205 of SEQ ID NO. 597, wherein said first amino acid sequence, bridging amino acid, second amino acid sequence and third amino acid sequence are contiguous and in a sequential order.

According to preferred embodiments of the present invention, there is provided an isolated chimeric polypeptide encoding for an edge portion of SEQ ID NO. 597, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IV, having a structure as follows: a sequence starting from any of amino acid numbers 157−x to 157; and ending at any of amino acid numbers 158+((n−2)−x), in which x varies from 0 to n−2.

According to preferred embodiments of the present invention, there is provided an isolated chimeric polypeptide encoding for SEQ ID NO. 597, comprising a first amino acid sequence being at least 90% homologous to amino acids 1-157 of Q9UJZ1 (SEQ ID NO:637), which also corresponds to amino acids 1-157 of SEQ ID NO. 597, and a second amino acid sequence being at least 90% homologous to amino acids 309-356 of Q9UJZ1 (SEQ ID NO:637), which also corresponds to amino acids 158-205 of SEQ ID NO. 597, wherein said first amino acid sequence and second amino acid sequence are contiguous and in a sequential order.

According to preferred embodiments of the present invention, there is provided an isolated chimeric polypeptide encoding for an edge portion of SEQ ID NO. 597, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IV, having a structure as follows: a sequence starting from any of amino acid numbers 157−x to 157; and ending at any of amino acid numbers 158+((n−2)−x), in which x varies from 0 to n−2.

According to preferred embodiments of the present invention, there is provided an isolated chimeric polypeptide encoding for SEQ ID NO. 598, comprising a first amino acid sequence being at least 70%, optionally at least 80%, preferably at least 85%, more preferably at least 90% and most preferably at least 95% homologous to a polypeptide sequence corresponding to amino acids 1-26 of SEQ ID NO. 598, a second amino acid sequence being at least 90% homologous to amino acids 13-128 of SEQ ID NO. 639, which also corresponds to amino acids 27-142 of SEQ ID NO. 598, and a third amino acid sequence being at least 70%, optionally at least 80%, preferably at least 85%, more preferably at least 90% and most preferably at least 95% homologous to a polypeptide sequence corresponding to amino acids 143-161 of SEQ ID NO. 598, wherein said first amino acid sequence, second amino acid sequence and third amino acid sequence are contiguous and in a sequential order.

According to preferred embodiments of the present invention, there is provided an isolated polypeptide encoding for a head of SEQ ID NO. 598, comprising a polypeptide being at least 70%, optionally at least about 80%, preferably at least about 85%, more preferably at least about 90% and most preferably at least about 95% homologous to amino acids 1-26 of SEQ ID NO. 598.

According to preferred embodiments of the present invention, there is provided an isolated polypeptide encoding for a tail of SEQ ID NO. 598, comprising a polypeptide being at least 70%, optionally at least about 80%, preferably at least about 85%, more preferably at least about 90% and most preferably at least about 95% homologous to amino in SEQ ID NO. 598.

According to preferred embodiments of the present invention, there is provided an isolated chimeric polypeptide encoding for SEQ ID NO. 598, comprising a first amino acid sequence being at least 90% homologous to amino acids 1-128 of SEQ ID NO. 638, which also corresponds to amino acids 1-128 of SEQ ID NO. 598, a bridging amino acid L corresponding to amino acid 129 of SEQ ID NO. 598, a second amino acid sequence being at least 90% homologous to amino acids 130-142 of SEQ ID NO. 638, which also corresponds to amino acids 130-142 of SEQ ID NO. 598, and a third amino acid sequence being at least 70%, optionally at least 80%, preferably at least 85%, more preferably at least 90% and most preferably at least 95% homologous to a polypeptide sequence corresponding to amino acids 143-161 of SEQ ID NO. 598, wherein said first amino acid sequence, bridging amino acid, second amino acid sequence and third amino acid sequence are contiguous and in a sequential order.

According to preferred embodiments of the present invention, there is provided an isolated polypeptide encoding for a tail of SEQ ID NO. 598, comprising a polypeptide being at least 70%, optionally at least about 80%, preferably at least about 85%, more preferably at least about 90% and most preferably at least about 95% homologous to amino in SEQ ID NO. 598.

According to preferred embodiments of the present invention, there is provided an isolated chimeric polypeptide encoding for SEQ ID NO. 598, comprising a first amino acid sequence being at least 90% homologous to amino acids 1-142 of Q9UJZ1 (SEQ ID NO:637), which also corresponds to amino acids 1-142 of SEQ ID NO. 598, and a second amino acid sequence being at least 70%, optionally at least 80%, preferably at least 85%, more preferably at least 90% and most preferably at least 95% homologous to a polypeptide sequence corresponding to amino acids 143-161 of SEQ ID NO. 598, wherein said first amino acid sequence and second amino acid sequence are contiguous and in a sequential order.

According to preferred embodiments of the present invention, there is provided an isolated polypeptide encoding for a tail of SEQ ID NO. 598, comprising a polypeptide being at least 70%, optionally at least about 80%, preferably at least about 85%, more preferably at least about 90% and most preferably at least about 95% homologous to amino in SEQ ID NO. 598.

According to preferred embodiments of the present invention, there is provided an isolated chimeric polypeptide encoding for SEQ ID NO. 600, comprising a first amino acid sequence being at least 90% homologous to amino acids 1-61 of SEQ ID NO. 638, which also corresponds to amino acids 1-61 of SEQ ID NO. 600, and a second amino acid sequence being at least 70%, optionally at least 80%, preferably at least 85%, more preferably at least 90% and most preferably at least 95% homologous to a polypeptide sequence corresponding to amino acids 62-102 of SEQ ID NO. 600, wherein said first amino acid sequence and second amino acid sequence are contiguous and in a sequential order.

According to preferred embodiments of the present invention, there is provided an isolated polypeptide encoding for a tail of SEQ ID NO. 600, comprising a polypeptide being at least 70%, optionally at least about 80%, preferably at least about 85%, more preferably at least about 90% and most preferably at least about 95% homologous to the sequence amino in SEQ ID NO. 600.

According to preferred embodiments of the present invention, there is provided an isolated chimeric polypeptide encoding for SEQ ID NO. 600, comprising a first amino acid sequence being at least 90% homologous to amino acids 1-61 of Q9UJZ1 (SEQ ID NO:637), which also corresponds to amino acids 1-61 of SEQ ID NO. 600, and a second amino acid sequence being at least 70%, optionally at least 80%, preferably at least 85%, more preferably at least 90% and most preferably at least 95% homologous to a polypeptide sequence corresponding to amino acids 62-102 of SEQ ID NO. 600, wherein said first amino acid sequence and second amino acid sequence are contiguous and in a sequential order.

According to preferred embodiments of the present invention, there is provided an isolated polypeptide encoding for a tail of SEQ ID NO. 600, comprising a polypeptide being at least 70%, optionally at least about 80%, preferably at least about 85%, more preferably at least about 90% and most preferably at least about 95% homologous to amino in SEQ ID NO. 600.

According to preferred embodiments of the present invention, there is provided an isolated chimeric polypeptide encoding for SEQ ID NO. 601, comprising a first amino acid sequence being at least 70%, optionally at least 80%, preferably at least 85%, more preferably at least 90% and most preferably at least 95% homologous to a polypeptide sequence corresponding to amino acids 1-26 of SEQ ID NO. 601, a second amino acid sequence being at least 90% homologous to amino acids 13-47 of SEQ ID NO. 639, which also corresponds to amino acids 27-61 of SEQ ID NO. 601, and a third amino acid sequence being at least 70%, optionally at least 80%, preferably at least 85%, more preferably at least 90% and most preferably at least 95% homologous to a polypeptide sequence corresponding to amino acids 62-72 of SEQ ID NO. 601, wherein said first amino acid sequence, second amino acid sequence and third amino acid sequence are contiguous and in a sequential order.

According to preferred embodiments of the present invention, there is provided an isolated polypeptide encoding for a head of SEQ ID NO. 601, comprising a polypeptide being at least 70%, optionally at least about 80%, preferably at least about 85%, more preferably at least about 90% and most preferably at least about 95% homologous to amino acids 1-26 of SEQ ID NO. 601.

According to preferred embodiments of the present invention, there is provided an isolated polypeptide encoding for a tail of SEQ ID NO. 601, comprising a polypeptide being at least 70%, optionally at least about 80%, preferably at least about 85%, more preferably at least about 90% and most preferably at least about 95% homologous to amino acids 62-72 in SEQ ID NO. 601.

According to preferred embodiments of the present invention, there is provided an isolated chimeric polypeptide encoding for SEQ ID NO. 601, comprising a first amino acid sequence being at least 90% homologous to amino acids 1-61 of Q96FY2 (SEQ ID NO:638), which also corresponds to amino acids 1-61 of SEQ ID NO. 601, and a second amino acid sequence being at least 70%, optionally at least 80%, preferably at least 85%, more preferably at least 90% and most preferably at least 95% homologous to a polypeptide sequence corresponding to amino acids 62-72 of SEQ ID NO. 601, wherein said first amino acid sequence and second amino acid sequence are contiguous and in a sequential order.

According to preferred embodiments of the present invention, there is provided an isolated polypeptide encoding for a tail of SEQ ID NO. 601, comprising a polypeptide being at least 70%, optionally at least about 80%, preferably at least about 85%, more preferably at least about 90% and most preferably at least about 95% homologous to amino acids 62-72 in SEQ ID NO. 601.

According to preferred embodiments of the present invention, there is provided an isolated chimeric polypeptide encoding for SEQ ID NO. 601, comprising a first amino acid sequence being at least 90% homologous to amino acids 1-61 of Q9UJZ1 (SEQ ID NO:637), which also corresponds to amino acids 1-61 of SEQ ID NO. 601, and a second amino acid sequence being at least 70%, optionally at least 80%, preferably at least 85%, more preferably at least 90% and most preferably at least 95% homologous to a polypeptide sequence corresponding to amino acids 62-72 of SEQ ID NO. 601, wherein said first amino acid sequence and second amino acid sequence are contiguous and in a sequential order.

According to preferred embodiments of the present invention, there is provided an isolated polypeptide encoding for a tail of SEQ ID NO. 601, comprising a polypeptide being at least 70%, optionally at least about 80%, preferably at least about 85%, more preferably at least about 90% and most preferably at least about 95% homologous to amino acids 62-72 in SEQ ID NO. 601.

According to preferred embodiments of the present invention, there is provided an isolated chimeric polypeptide encoding for SEQ ID NO. 602, comprising a first amino acid sequence being at least 70%, optionally at least 80%, preferably at least 85%, more preferably at least 90% and most preferably at least 95% homologous to a polypeptide sequence corresponding to amino acids 1-26 of SEQ ID NO. 602, a second amino acid sequence being at least 90% homologous to amino acids 13-80 of SEQ ID NO. 639, which also corresponds to amino acids 27-94 of SEQ ID NO. 602, and a third amino acid sequence being at least 70%, optionally at least 80%, preferably at least 85%, more preferably at least 90% and most preferably at least 95% homologous to a polypeptide sequence corresponding to amino acids 95-111 of SEQ ID NO. 602, wherein said first amino acid sequence, second amino acid sequence and third amino acid sequence are contiguous and in a sequential order.

According to preferred embodiments of the present invention, there is provided an isolated polypeptide encoding for a head of SEQ ID NO. 602, comprising a polypeptide being at least 70%, optionally at least about 80%, preferably at least about 85%, more preferably at least about 90% and most preferably at least about 95% homologous to amino acids 1-26 of SEQ ID NO. 602.

According to preferred embodiments of the present invention, there is provided an isolated polypeptide encoding for a tail of SEQ ID NO. 602, comprising a polypeptide being at least 70%, optionally at least about 80%, preferably at least about 85%, more preferably at least about 90% and most preferably at least about 95% homologous to amino in SEQ ID NO. 602.

According to preferred embodiments of the present invention, there is provided an isolated chimeric polypeptide encoding for SEQ ID NO. 602, comprising a first amino acid sequence being at least 90% homologous to amino acids 1-94 of SEQ ID NO. 638, which also corresponds to amino acids 1-94 of SEQ ID NO. 602, and a second amino acid sequence being at least 70%, optionally at least 80%, preferably at least 85%, more preferably at least 90% and most preferably at least 95% homologous to a polypeptide sequence corresponding to amino acids 95-111 of SEQ ID NO. 602, wherein said first amino acid sequence and second amino acid sequence are contiguous and in a sequential order.

According to preferred embodiments of the present invention, there is provided an isolated polypeptide encoding for a tail of SEQ ID NO. 602, comprising a polypeptide being at least 70%, optionally at least about 80%, preferably at least about 85%, more preferably at least about 90% and most preferably at least about 95% homologous to amino in SEQ ID NO. 602.

According to preferred embodiments of the present invention, there is provided an isolated chimeric polypeptide encoding for SEQ ID NO. 602, comprising a first amino acid sequence being at least 90% homologous to amino acids 1-94 of Q9UJZ1 (SEQ ID NO:637), which also corresponds to amino acids 1-94 of SEQ ID NO. 602, and a second amino acid sequence being at least 70%, optionally at least 80%, preferably at least 85%, more preferably at least 90% and most preferably at least 95% homologous to a polypeptide sequence corresponding to amino acids 95-111 of SEQ ID NO. 602, wherein said first amino acid sequence and second amino acid sequence are contiguous and in a sequential order.

According to preferred embodiments of the present invention, there is provided an isolated polypeptide encoding for a tail of SEQ ID NO. 602, comprising a polypeptide being at least 70%, optionally at least about 80%, preferably at least about 85%, more preferably at least about 90% and most preferably at least about 95% homologous to amino in SEQ ID NO. 602.

According to preferred embodiments of the present invention, there is provided an isolated chimeric polypeptide encoding for SEQ ID NO. 581, comprising a first amino acid sequence being at least 90% homologous to amino acids 1-67 of PLTP_HUMAN (SEQ ID NO:636), which also corresponds to amino acids 1-67 of SEQ ID NO. 581, and a second amino acid sequence being at least 90% homologous to amino acids 163-493 of PLTP_HUMAN (SEQ ID NO:636), which also corresponds to amino acids 68-398 of SEQ ID NO. 581, wherein said first amino acid sequence and second amino acid sequence are contiguous and in a sequential order.

According to preferred embodiments of the present invention, there is provided an isolated chimeric polypeptide encoding for an edge portion of SEQ ID NO. 581, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EK, having a structure as follows: a sequence starting from any of amino acid numbers 67−x to 67; and ending at any of amino acid numbers 68+((n−2)−x), in which x varies from 0 to n−2.

According to preferred embodiments of the present invention, there is provided an isolated chimeric polypeptide encoding for SEQ ID NO. 582, comprising a first amino acid sequence being at least 90% homologous to amino acids 1-427 of PLTP_HUMAN (SEQ ID NO:636), which also corresponds to amino acids 1-427 of SEQ ID NO. 582, and a second amino acid sequence being at least 70%, optionally at least 80%, preferably at least 85%, more preferably at least 90% and most preferably at least 95% homologous to a polypeptide sequence corresponding to amino acids 428-432 of SEQ ID NO. 582, wherein said first amino acid sequence and second amino acid sequence are contiguous and in a sequential order.

According to preferred embodiments of the present invention, there is provided an isolated polypeptide encoding for a tail of SEQ ID NO. 582, comprising a polypeptide being at least 70%, optionally at least about 80%, preferably at least about 85%, more preferably at least about 90% and most preferably at least about 95% homologous to amino in SEQ ID NO. 582.

According to preferred embodiments of the present invention, there is provided an isolated chimeric polypeptide encoding for SEQ ID NO. 584, comprising a first amino acid sequence being at least 90% homologous to amino acids 1-67 of PLTP_HUMAN (SEQ ID NO:636), which also corresponds to amino acids 1-67 of SEQ ID NO. 584, and a second amino acid sequence being at least 70%, optionally at least 80%, preferably at least 85%, more preferably at least 90% and most preferably at least 95% homologous to a polypeptide sequence corresponding to amino acids 68-98 of SEQ ID NO. 584, wherein said first amino acid sequence and second amino acid sequence are contiguous and in a sequential order.

According to preferred embodiments of the present invention, there is provided an isolated polypeptide encoding for a tail of SEQ ID NO. 584, comprising a polypeptide being at least 70%, optionally at least about 80%, preferably at least about 85%, more preferably at least about 90% and most preferably at least about 95% homologous to amino acids 68-98 in SEQ ID NO. 584.

According to preferred embodiments of the present invention, there is provided an isolated chimeric polypeptide encoding for SEQ ID NO. 585, comprising a first amino acid sequence being at least 90% homologous to amino acids 1-183 of PLTP_HUMAN (SEQ ID NO:636), which also corresponds to amino acids 1-183 of SEQ ID NO. 585, and a second amino acid sequence being at least 70%, optionally at least 80%, preferably at least 85%, more preferably at least 90% and most preferably at least 95% homologous to a polypeptide sequence corresponding to amino acids 184-200 of SEQ ID NO. 585, wherein said first amino acid sequence and second amino acid sequence are contiguous and in a sequential order.

According to preferred embodiments of the present invention, there is provided an isolated polypeptide encoding for a tail of SEQ ID NO. 585, comprising a polypeptide being at least 70%, optionally at least about 80%, preferably at least about 85%, more preferably at least about 90% and most preferably at least about 95% homologous to amino in SEQ ID NO. 585.

According to preferred embodiments of the present invention, there is provided an isolated chimeric polypeptide encoding for SEQ ID NO. 586, comprising a first amino acid sequence being at least 90% homologous to amino acids 1-205 of PLTP_HUMAN (SEQ ID NO:636), which also corresponds to amino acids 1-205 of SEQ ID NO. 586, and a second amino acid sequence being at least 70%, optionally at least 80%, preferably at least 85%, more preferably at least 90% and most preferably at least 95% homologous to a polypeptide sequence corresponding to amino acids 206-217 of SEQ ID NO. 586, wherein said first amino acid sequence and second amino acid sequence are contiguous and in a sequential order.

According to preferred embodiments of the present invention, there is provided an isolated polypeptide encoding for a tail of SEQ ID NO. 586, comprising a polypeptide being at least 70%, optionally at least about 80%, preferably at least about 85%, more preferably at least about 90% and most preferably at least about 95% homologous to amino in SEQ ID NO. 586.

According to preferred embodiments of the present invention, there is provided an isolated chimeric polypeptide encoding for SEQ ID NO. 587, comprising a first amino acid sequence being at least 90% homologous to amino acids 1-109 of PLTP_HUMAN (SEQ ID NO:636), which also corresponds to amino acids 1-109 of SEQ ID NO. 587, a second amino acid sequence bridging amino acid sequence comprising of L, a third amino acid sequence being at least 90% homologous to amino acids 163-183 of PLTP_HUMAN (SEQ ID NO:636), which also corresponds to amino acids 111-131 of SEQ ID NO. 587, and a fourth amino acid sequence being at least 70%, optionally at least 80%, preferably at least 85%, more preferably at least 90% and most preferably at least 95% homologous to a polypeptide sequence corresponding to amino acids 132-148 of SEQ ID NO. 587, wherein said first amino acid sequence, second amino acid sequence, third amino acid sequence and fourth amino acid sequence are contiguous and in a sequential order.

According to preferred embodiments of the present invention, there is provided an isolated polypeptide encoding for an edge portion of SEQ ID NO. 587, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least three amino acids comprise FLK having a structure as follows (numbering according to SEQ ID NO. 587): a sequence starting from any of amino acid numbers 109−x to 109; and ending at any of amino acid numbers 111+((n−2)−x), in which x varies from 0 to n−2.

According to preferred embodiments of the present invention, there is provided an isolated polypeptide encoding for a tail of SEQ ID NO. 587, comprising a polypeptide being at least 70%, optionally at least about 80%, preferably at least about 85%, more preferably at least about 90% and most preferably at least about 95% homologous to amino in SEQ ID NO. 587.

According to preferred embodiments of the present invention, there is provided an isolated chimeric polypeptide encoding for SEQ ID NO. 576, comprising a first amino acid sequence being at least 90% homologous to amino acids 1-1056 of SEQ ID NO. 634, which also corresponds to amino acids 1-1056 of SEQ ID NO. 576, and a second amino acid sequence being at least 70%, optionally at least 80%, preferably at least 85%, more preferably at least 90% and most preferably at least 95% homologous to a polypeptide sequence corresponding to amino acids 1057-1081 of SEQ ID NO. 576, wherein said first amino acid sequence and second amino acid sequence are contiguous and in a sequential order.

According to preferred embodiments of the present invention, there is provided an isolated polypeptide encoding for a tail of SEQ ID NO. 576, comprising a polypeptide being at least 70%, optionally at least about 80%, preferably at least about 85%, more preferably at least about 90% and most preferably at least about 95% homologous to amino acids 1057-1081 in SEQ ID NO. 576.

According to preferred embodiments of the present invention, there is provided an isolated chimeric polypeptide encoding for SEQ ID NO. 577, comprising a first amino acid sequence being at least 90% homologous to amino acids 1-714 of SEQ ID NO. 634, which also corresponds to amino acids 1-714 of SEQ ID NO. 577, and a second amino acid sequence being at least 70%, optionally at least 80%, preferably at least 85%, more preferably at least 90% and most preferably at least 95% homologous to a polypeptide sequence corresponding to amino acids 715-729 of SEQ ID NO. 577, wherein said first amino acid sequence and second amino acid sequence are contiguous and in a sequential order.

According to preferred embodiments of the present invention, there is provided an isolated polypeptide encoding for a tail of SEQ ID NO. 577, comprising a polypeptide being at least 70%, optionally at least about 80%, preferably at least about 85%, more preferably at least about 90% and most preferably at least about 95% homologous to amino in SEQ ID NO. 577.

According to preferred embodiments of the present invention, there is provided an isolated chimeric polypeptide encoding for SEQ ID NO. 578, comprising a first amino acid sequence being at least 90% homologous to amino acids 1-648 of SEQ ID NO. 634, which also corresponds to amino acids 1-648 of SEQ ID NO. 578, a second amino acid sequence being at least 90% homologous to amino acids 667-714 of SEQ ID NO. 634, which also corresponds to amino acids 649-696 of SEQ ID NO. 578, and a third amino acid sequence being at least 70%, optionally at least 80%, preferably at least 85%, more preferably at least 90% and most preferably at least 95% homologous to a polypeptide sequence corresponding to amino acids 697-738 of SEQ ID NO. 578, wherein said first amino acid sequence, second amino acid sequence and third amino acid sequence are contiguous and in a sequential order.

According to preferred embodiments of the present invention, there is provided an isolated chimeric polypeptide encoding for an edge portion of SEQ ID NO. 578, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AG, having a structure as follows: a sequence starting from any of amino acid numbers 648−x to 648; and ending at any of amino acid numbers 649+((n−2)−x), in which x varies from 0 to n−2.

According to preferred embodiments of the present invention, there is provided an isolated polypeptide encoding for a tail of SEQ ID NO. 578, comprising a polypeptide being at least 70%, optionally at least about 80%, preferably at least about 85%, more preferably at least about 90% and most preferably at least about 95% homologous to amino in SEQ ID NO. 578.

According to preferred embodiments of the present invention, there is provided an isolated chimeric polypeptide encoding for SEQ ID NO. 579, comprising a first amino acid sequence being at least 90% homologous to amino acids 1-260 of SEQ ID NO. 634, which also corresponds to amino acids 1-260 of SEQ ID NO. 579, and a second amino acid sequence being at least 70%, optionally at least 80%, preferably at least 85%, more preferably at least 90% and most preferably at least 95% homologous to a polypeptide sequence corresponding to amino acids 261-273 of SEQ ID NO. 579, wherein said first amino acid sequence and second amino acid sequence are contiguous and in a sequential order.

According to preferred embodiments of the present invention, there is provided an isolated polypeptide encoding for a tail of SEQ ID NO. 579, comprising a polypeptide being at least 70%, optionally at least about 80%, preferably at least about 85%, more preferably at least about 90% and most preferably at least about 95% homologous to amino in SEQ ID NO. 579.

According to preferred embodiments of the present invention, there is provided an isolated chimeric polypeptide encoding for SEQ ID NO. 575, comprising a first amino acid sequence being at least 90% homologous to amino acids 1-13 of GFR2_HUMAN (SEQ ID NO:632), which also corresponds to amino acids 1-13 of SEQ ID NO. 575, and a second amino acid sequence being at least 70%, optionally at least 80%, preferably at least 85%, more preferably at least 90% and most preferably at least 95% homologous to a polypeptide sequence corresponding to amino acids 14-30 of SEQ ID NO. 575, wherein said first amino acid sequence and second amino acid sequence are contiguous and in a sequential order.

According to preferred embodiments of the present invention, there is provided an isolated polypeptide encoding for a tail of SEQ ID NO. 575, comprising a polypeptide being at least 70%, optionally at least about 80%, preferably at least about 85%, more preferably at least about 90% and most preferably at least about 95% homologous to amino acids 14-30 in SEQ ID NO. 575.

According to preferred embodiments of the present invention, there is provided an isolated chimeric polypeptide encoding for SEQ ID NO. 567, comprising a first amino acid sequence being at least 90% homologous to amino acids 1-123 of SEQ ID NO. 631, which also corresponds to amino acids 1-123 of SEQ ID NO. 567, and a second amino acid sequence being at least 70%, optionally at least 80%, preferably at least 85%, more preferably at least 90% and most preferably at least 95% homologous to a polypeptide sequence corresponding to amino acids 124-156 of SEQ ID NO. 567, wherein said first amino acid sequence and second amino acid sequence are contiguous and in a sequential order.

According to preferred embodiments of the present invention, there is provided an isolated polypeptide encoding for a tail of SEQ ID NO. 567, comprising a polypeptide being at least 70%, optionally at least about 80%, preferably at least about 85%, more preferably at least about 90% and most preferably at least about 95% homologous to amino in SEQ ID NO. 567.

According to preferred embodiments of the present invention, there is provided an isolated chimeric polypeptide encoding for SEQ ID NO. 567, comprising a first amino acid sequence being at least 70%, optionally at least 80%, preferably at least 85%, more preferably at least 90% and most preferably at least 95% homologous to a polypeptide sequence corresponding to amino acids 1-73 of SEQ ID NO. 567, and a second amino acid sequence being at least 90% homologous to amino acids 1799-1881 of SEQ ID NO. 629, which also corresponds to amino acids 74-156 of SEQ ID NO. 567, wherein said first amino acid sequence and second amino acid sequence are contiguous and in a sequential order.

According to preferred embodiments of the present invention, there is provided an isolated polypeptide encoding for a head of SEQ ID NO. 567, comprising a polypeptide being at least 70%, optionally at least about 80%, preferably at least about 85%, more preferably at least about 90% and most preferably at least about 95% homologous to the sequence amino acids 1-73 of SEQ ID NO. 567.

According to preferred embodiments of the present invention, there is provided an isolated chimeric polypeptide encoding for SEQ ID NO. 567, comprising a first amino acid sequence being at least 90% homologous to amino acids 1-52 of SEQ ID NO. 630, which also corresponds to amino acids 1-52 of SEQ ID NO. 567, a bridging amino acid G corresponding to amino acid 53 of SEQ ID NO. 567, a second amino acid sequence being at least 90% homologous to amino acids 54-124 of SEQ ID NO. 630, which also corresponds to amino acids 54-124 of SEQ ID NO. 567, and a third amino acid sequence being at least 70%, optionally at least 80%, preferably at least 85%, more preferably at least 90% and most preferably at least 95% homologous to a polypeptide sequence corresponding to amino acids 125-156 of SEQ ID NO. 567, wherein said first amino acid sequence, bridging amino acid, second amino acid sequence and third amino acid sequence are contiguous and in a sequential order.

According to preferred embodiments of the present invention, there is provided an isolated polypeptide encoding for a tail of SEQ ID NO. 567, comprising a polypeptide being at least 70%, optionally at least about 80%, preferably at least about 85%, more preferably at least about 90% and most preferably at least about 95% homologous to amino in SEQ ID NO. 567.

According to preferred embodiments of the present invention, there is provided an isolated chimeric polypeptide encoding for SEQ ID NO. 568, comprising a first amino acid sequence being at least 90% homologous to amino acids 1-123 of SEQ ID NO. 631, which also corresponds to amino acids 1-123 of SEQ ID NO. 568, and a second amino acid sequence being at least 70%, optionally at least 80%, preferably at least 85%, more preferably at least 90% and most preferably at least 95% homologous to a polypeptide sequence corresponding to amino acids 124-169 of SEQ ID NO. 568, wherein said first amino acid sequence and second amino acid sequence are contiguous and in a sequential order.

According to preferred embodiments of the present invention, there is provided an isolated polypeptide encoding for a tail of SEQ ID NO. 568, comprising a polypeptide being at least 70%, optionally at least about 80%, preferably at least about 85%, more preferably at least about 90% and most preferably at least about 95% homologous to amino in SEQ ID NO. 568.

According to preferred embodiments of the present invention, there is provided an isolated chimeric polypeptide encoding for SEQ ID NO. 568, comprising a first amino acid sequence being at least 90% homologous to amino acids 1-52 of SEQ ID NO. 630, which also corresponds to amino acids 1-52 of SEQ ID NO. 568, a bridging amino acid G corresponding to amino acid 53 of SEQ ID NO. 568, a second amino acid sequence being at least 90% homologous to amino acids 54-122 of SEQ ID NO. 630, which also corresponds to amino acids 54-122 of SEQ ID NO. 568, a third amino acid sequence being at least 70%, optionally at least 80%, preferably at least 85%, more preferably at least 90% and most preferably at least 95% homologous to a polypeptide sequence corresponding to amino acids 123-136 of SEQ ID NO. 568, and a fourth amino acid sequence being at least 90% homologous to amino acids 123-155 of SEQ ID NO. 630, which also corresponds to amino acids 137-169 of SEQ ID NO. 568, wherein said first amino acid sequence, bridging amino acid, second amino acid sequence, third amino acid sequence and fourth amino acid sequence are contiguous and in a sequential order.

According to preferred embodiments of the present invention, there is provided an isolated polypeptide encoding for an edge portion of SEQ ID NO. 568, comprising an amino acid sequence being at least 70%, optionally at least about 80%, preferably at least about 85%, more preferably at least about 90% and most preferably at least about 95% homologous to the sequence encoding for amino acids 123-136, corresponding to SEQ ID NO. 568.

According to preferred embodiments of the present invention, there is provided an isolated chimeric polypeptide encoding for SEQ ID NO. 569, comprising a first amino acid sequence being at least 90% homologous to amino acids 1-123 of SEQ ID NO. 631, which also corresponds to amino acids 1-123 of SEQ ID NO. 569, and a second amino acid sequence being at least 70%, optionally at least 80%, preferably at least 85%, more preferably at least 90% and most preferably at least 95% homologous to a polypeptide sequence corresponding to amino acids 124-180 of SEQ ID NO. 569, wherein said first amino acid sequence and second amino acid sequence are contiguous and in a sequential order.

According to preferred embodiments of the present invention, there is provided an isolated polypeptide encoding for a tail of SEQ ID NO. 569, comprising a polypeptide being at least 70%, optionally at least about 80%, preferably at least about 85%, more preferably at least about 90% and most preferably at least about 95% homologous to the sequence amino in SEQ ID NO. 569.

According to preferred embodiments of the present invention, there is provided an isolated chimeric polypeptide encoding for SEQ ID NO. 569, comprising a first amino acid sequence being at least 90% homologous to amino acids 1-52 of SEQ ID NO. 630, which also corresponds to amino acids 1-52 of SEQ ID NO. 569, a bridging amino acid G corresponding to amino acid 53 of SEQ ID NO. 569, a second amino acid sequence being at least 90% homologous to amino acids 54-123 of SEQ ID NO. 630, which also corresponds to amino acids 54-123 of SEQ ID NO. 569, a third amino acid sequence being at least 70%, optionally at least 80%, preferably at least 85%, more preferably at least 90% and most preferably at least 95% homologous to a polypeptide sequence corresponding to amino acids 124-148 of SEQ ID NO. 569, and a fourth amino acid sequence being at least 90% homologous to amino acids 124-155 of SEQ ID NO. 630, which also corresponds to amino acids 149-180 of SEQ ID NO. 569, wherein said first amino acid sequence, bridging amino acid, second amino acid sequence, third amino acid sequence and fourth amino acid sequence are contiguous and in a sequential order.

According to preferred embodiments of the present invention, there is provided an isolated polypeptide encoding for an edge portion of SEQ ID NO. 569, comprising an amino acid sequence being at least 70%, optionally at least about 80%, preferably at least about 85%, more preferably at least about 90% and most preferably at least about 95% homologous to the sequence encoding for amino acids 124-148, corresponding to SEQ ID NO. 569.

According to preferred embodiments of the present invention, there is provided an isolated chimeric polypeptide encoding for SEQ ID NO. 570, comprising a first amino acid sequence being at least 90% homologous to amino acids 1-123 of SEQ ID NO. 631, which also corresponds to amino acids 1-123 of SEQ ID NO. 570, and a second amino acid sequence being at least 70%, optionally at least 80%, preferably at least 85%, more preferably at least 90% and most preferably at least 95% homologous to a polypeptide sequence corresponding to amino acids 124-145 of SEQ ID NO. 570, wherein said first amino acid sequence and second amino acid sequence are contiguous and in a sequential order.

According to preferred embodiments of the present invention, there is provided an isolated polypeptide encoding for a tail of SEQ ID NO. 570, comprising a polypeptide being at least 70%, optionally at least about 80%, preferably at least about 85%, more preferably at least about 90% and most preferably at least about 95% homologous to amino in SEQ ID NO. 570.

According to preferred embodiments of the present invention, there is provided an isolated chimeric polypeptide encoding for SEQ ID NO. 570, comprising a first amino acid sequence being at least 90% homologous to amino acids 1-52 of SEQ ID NO. 630, which also corresponds to amino acids 1-52 of SEQ ID NO. 570, a bridging amino acid G corresponding to amino acid 53 of SEQ ID NO. 570, a second amino acid sequence being at least 90% homologous to amino acids 54-124 of SEQ ID NO. 630, which also corresponds to amino acids 54-124 of SEQ ID NO. 570, and a third amino acid sequence being at least 70%, optionally at least 80%, preferably at least 85%, more preferably at least 90% and most preferably at least 95% homologous to a polypeptide sequence corresponding to amino acids 125-145 of SEQ ID NO. 570, wherein said first amino acid sequence, bridging amino acid, second amino acid sequence and third amino acid sequence are contiguous and in a sequential order.

According to preferred embodiments of the present invention, there is provided an isolated polypeptide encoding for a tail of SEQ ID NO. 570, comprising a polypeptide being at least 70%, optionally at least about 80%, preferably at least about 85%, more preferably at least about 90% and most preferably at least about 95% homologous to amino in SEQ ID NO. 570.

According to preferred embodiments of the present invention, there is provided an isolated chimeric polypeptide encoding for SEQ ID NO. 571, comprising a first amino acid sequence being at least 90% homologous to amino acids 1-101 of SEQ ID NO. 631, which also corresponds to amino acids 1-101 of SEQ ID NO. 571, and a second amino acid sequence being at least 70%, optionally at least 80%, preferably at least 85%, more preferably at least 90% and most preferably at least 95% homologous to a polypeptide sequence corresponding to amino acids 102-122 of SEQ ID NO. 571, wherein said first amino acid sequence and second amino acid sequence are contiguous and in a sequential order.

According to preferred embodiments of the present invention, there is provided an isolated polypeptide encoding for a tail of SEQ ID NO. 571, comprising a polypeptide being at least 70%, optionally at least about 80%, preferably at least about 85%, more preferably at least about 90% and most preferably at least about 95% homologous to amino in SEQ ID NO. 571.

According to preferred embodiments of the present invention, there is provided an isolated chimeric polypeptide encoding for SEQ ID NO. 571, comprising a first amino acid sequence being at least 90% homologous to amino acids 1-52 of SEQ ID NO. 630, which also corresponds to amino acids 1-52 of SEQ ID NO. 571, a bridging amino acid G corresponding to amino acid 53 of SEQ ID NO. 571, a second amino acid sequence being at least 90% homologous to amino acids 54-101 of SEQ ID NO. 630, which also corresponds to amino acids 54-101 of SEQ ID NO. 571, and a third amino acid sequence being at least 70%, optionally at least 80%, preferably at least 85%, more preferably at least 90% and most preferably at least 95% homologous to a polypeptide sequence corresponding to amino acids 102-122 of SEQ ID NO. 571, wherein said first amino acid sequence, bridging amino acid, second amino acid sequence and third amino acid sequence are contiguous and in a sequential order.

According to preferred embodiments of the present invention, there is provided an isolated polypeptide encoding for a tail of SEQ ID NO. 571, comprising a polypeptide being at least 70%, optionally at least about 80%, preferably at least about 85%, more preferably at least about 90% and most preferably at least about 95% homologous to amino in SEQ ID NO. 571.

According to preferred embodiments of the present invention, there is provided an isolated chimeric polypeptide encoding for SEQ ID NO. 572, comprising a first amino acid sequence being at least 90% homologous to amino acids 1-62 of SEQ ID NO. 631, which also corresponds to amino acids 1-62 of SEQ ID NO. 572, a bridging amino acid P corresponding to amino acid 63 of SEQ ID NO. 572, a second amino acid sequence being at least 90% homologous to amino acids 64-123 of SEQ ID NO. 631, which also corresponds to amino acids 64-123 of SEQ ID NO. 572, and a third amino acid sequence being at least 70%, optionally at least 80%, preferably at least 85%, more preferably at least 90% and most preferably at least 95% homologous to a polypeptide sequence corresponding to amino acids 124-155 of SEQ ID NO. 572, wherein said first amino acid sequence, bridging amino acid, second amino acid sequence and third amino acid sequence are contiguous and in a sequential order.

According to preferred embodiments of the present invention, there is provided an isolated polypeptide encoding for a tail of SEQ ID NO. 572, comprising a polypeptide being at least 70%, optionally at least about 80%, preferably at least about 85%, more preferably at least about 90% and most preferably at least about 95% homologous to amino in SEQ ID NO. 572.

According to preferred embodiments of the present invention, there is provided an isolated chimeric polypeptide encoding for SEQ ID NO. 572, comprising a first amino acid sequence being at least 90% homologous to amino acids 1-52 of SEQ ID NO. 630, which also corresponds to amino acids 1-52 of SEQ ID NO. 572, a bridging amino acid G corresponding to amino acid 53 of SEQ ID NO. 572, a second amino acid sequence being at least 90% homologous to LSDDEETIS corresponding to amino acids 54-62 of SEQ ID NO. 630, which also corresponds to amino acids 54-62 of SEQ ID NO. 572, a bridging amino acid P corresponding to amino acid 63 of SEQ ID NO. 572, and a third amino acid sequence being at least 90% homologous to amino acids 64-155 of SEQ ID NO. 630, which also corresponds to amino acids 64-155 of SEQ ID NO. 572, wherein said first amino acid sequence, bridging amino acid, second amino acid sequence, bridging amino acid and third amino acid sequence are contiguous and in a sequential order.

According to preferred embodiments of the present invention, there is provided an isolated chimeric polypeptide encoding for SEQ ID NO. 573 comprising a first amino acid sequence being at least 90% homologous to amino acids 1-62 of SEQ ID NO. 631 which also corresponds to amino acids 1-62 of SEQ ID NO. 573, a bridging amino acid P corresponding to amino acid 63 of SEQ ID NO. 573, a second amino acid sequence being at least 90% homologous to amino acids 64-101 of SEQ ID NO. 631, which also corresponds to amino acids 64-101 of SEQ ID NO. 573, and a third amino acid sequence being at least 70%, optionally at least 80%, preferably at least 85%, more preferably at least 90% and most preferably at least 95% homologous to a polypeptide sequence corresponding to amino acids 102-109 of SEQ ID NO. 573, wherein said first amino acid sequence, bridging amino acid, second amino acid sequence and third amino acid sequence are contiguous and in a sequential order.

According to preferred embodiments of the present invention, there is provided an isolated polypeptide encoding for a tail of SEQ ID NO. 573, comprising a polypeptide being at least 70%, optionally at least about 80%, preferably at least about 85%, more preferably at least about 90% and most preferably at least about 95% homologous to amino in SEQ ID NO. 573.

According to preferred embodiments of the present invention, there is provided an isolated chimeric polypeptide encoding for SEQ ID NO. 573, comprising a first amino acid sequence being at least 90% homologous to amino acids 1-52 of SEQ ID NO. 630 which also corresponds to amino acids 1-52 of SEQ ID NO. 573, a bridging amino acid G corresponding to amino acid 53 of SEQ ID NO. 573, a second amino acid sequence being at least 90% homologous to amino acids 54-62 of SEQ ID NO. 630, which also corresponds to amino acids 54-62 of SEQ ID NO. 573, a bridging amino acid P corresponding to amino acid 63 of SEQ ID NO. 573, a third amino acid sequence being at least 90% homologous to amino acids 64-101 of SEQ ID NO. 630, which also corresponds to amino acids 64-101 of SEQ ID NO. 573, and a fourth amino acid sequence being at least 70%, optionally at least 80%, preferably at least 85%, more preferably at least 90% and most preferably at least 95% homologous to a polypeptide sequence corresponding to amino acids 102-109 of SEQ ID NO. 573, wherein said first amino acid sequence, bridging amino acid, second amino acid sequence, bridging amino acid, third amino acid sequence and fourth amino acid sequence are contiguous and in a sequential order.

According to preferred embodiments of the present invention, there is provided an isolated polypeptide encoding for a tail of SEQ ID NO. 573, comprising a polypeptide being at least 70%, optionally at least about 80%, preferably at least about 85%, more preferably at least about 90% and most preferably at least about 95% homologous to amino in SEQ ID NO. 573.

According to preferred embodiments of the present invention, there is provided an isolated chimeric polypeptide encoding for SEQ ID NO. 574, comprising a first amino acid sequence being at least 90% homologous to amino acids 1-62 of SEQ ID NO. 631, which also corresponds to amino acids 1-62 of SEQ ID NO. 574, a bridging amino acid P corresponding to amino acid 63 of SEQ ID NO. 574, a second amino acid sequence being at least 90% homologous to amino acids 64-101 of SEQ ID NO. 631, which also corresponds to amino acids 64-101 of SEQ ID NO. 574, and a third amino acid sequence being at least 70%, optionally at least 80%, preferably at least 85%, more preferably at least 90% and most preferably at least 95% homologous to a polypeptide sequence corresponding to amino acids 102-133 of SEQ ID NO. 574, wherein said first amino acid sequence, bridging amino acid, second amino acid sequence and third amino acid sequence are contiguous and in a sequential order.

According to preferred embodiments of the present invention, there is provided an isolated polypeptide encoding for a tail of SEQ ID NO. 574, comprising a polypeptide being at least 70%, optionally at least about 80%, preferably at least about 85%, more preferably at least about 90% and most preferably at least about 95% homologous to amino in SEQ ID NO. 574.

According to preferred embodiments of the present invention, there is provided an isolated chimeric polypeptide encoding for SEQ ID NO. 574, comprising a first amino acid sequence being at least 90% homologous to amino acids 1-52 of SEQ ID NO. 630, which also corresponds to amino acids 1-52 of SEQ ID NO. 574, a bridging amino acid G corresponding to amino acid 53 of SEQ ID NO. 574, a second amino acid sequence being at least 90% homologous to amino acids 54-62 of SEQ ID NO. 630, which also corresponds to amino acids 54-62 of SEQ ID NO. 574, a bridging amino acid P corresponding to amino acid 63 of SEQ ID NO. 574, a third amino acid sequence being at least 90% homologous to amino acids 64-101 of SEQ ID NO. 630, which also corresponds to amino acids 64-101 of SEQ ID NO. 574, and a fourth amino acid sequence being at least 90% homologous to amino acids 124-155 of SEQ ID NO. 630, which also corresponds to amino acids 102-133 of SEQ ID NO. 574, wherein said first amino acid sequence, bridging amino acid, second amino acid sequence, bridging amino acid, third amino acid sequence and fourth amino acid sequence are contiguous and in a sequential order.

According to preferred embodiments of the present invention, there is provided an isolated chimeric polypeptide encoding for an edge portion of SEQ ID NO. 574, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KV, having a structure as follows: a sequence starting from any of amino acid numbers 101−x to 101; and ending at any of amino acid numbers 102+((n−2)−x), in which x varies from 0 to n−2.

According to preferred embodiments of the present invention, there is provided an isolated chimeric polypeptide encoding for SEQ ID NO. 564, comprising a first amino acid sequence being at least 90% homologous to amino acids 1-1617 of SEQ ID NO. 627, which also corresponds to amino acids 1-1617 of SEQ ID NO. 564, and a second amino acid sequence being at least 70%, optionally at least 80%, preferably at least 85%, more preferably at least 90% and most preferably at least 95% homologous to a polypeptide sequence corresponding to amino acids 1618-1645 of SEQ ID NO. 564, wherein said first amino acid sequence and second amino acid sequence are contiguous and in a sequential order.

According to preferred embodiments of the present invention, there is provided an isolated polypeptide encoding for a tail of SEQ ID NO. 564, comprising a polypeptide being at least 70%, optionally at least about 80%, preferably at least about 85%, more preferably at least about 90% and most preferably at least about 95% homologous to amino acids 1618-1645 in SEQ ID NO. 564.

According to preferred embodiments of the present invention, there is provided an isolated chimeric polypeptide encoding for SEQ ID NO. 565, comprising a first amino acid sequence being at least 90% homologous to amino acids 1-2062 of SEQ ID NO. 627, which also corresponds to amino acids 1-2062 of SEQ ID NO. 565, and a second amino acid sequence being at least 70%, optionally at least 80%, preferably at least 85%, more preferably at least 90% and most preferably at least 95% homologous to a polypeptide sequence corresponding to amino acids 2063-2074 of SEQ ID NO. 565, wherein said first amino acid sequence and second amino acid sequence are contiguous and in a sequential order.

According to preferred embodiments of the present invention, there is provided an isolated polypeptide encoding for a tail of SEQ ID NO. 565, comprising a polypeptide being at least 70%, optionally at least about 80%, preferably at least about 85%, more preferably at least about 90% and most preferably at least about 95% homologous to amino acids 2063-2074 in SEQ ID NO. 565.

According to preferred embodiments of the present invention, there is provided an isolated chimeric polypeptide encoding for SEQ ID NO. 566, comprising a first amino acid sequence being at least 90% homologous to amino acids 1-587 of SEQ ID NO. 627, which also corresponds to amino acids 1-587 of SEQ ID NO. 566, and a second amino acid sequence being at least 70%, optionally at least 80%, preferably at least 85%, more preferably at least 90% and most preferably at least 95% homologous to a polypeptide sequence corresponding to amino acids 588-603 of SEQ ID NO. 566, wherein said first amino acid sequence and second amino acid sequence are contiguous and in a sequential order.

According to preferred embodiments of the present invention, there is provided an isolated polypeptide encoding for a tail of SEQ ID NO. 566, comprising a polypeptide being at least 70%, optionally at least about 80%, preferably at least about 85%, more preferably at least about 90% and most preferably at least about 95% homologous to amino in SEQ ID NO. 566.

According to preferred embodiments of the present invention, there is provided an isolated chimeric polypeptide encoding for SEQ ID NO. 560, comprising a first amino acid sequence being at least 90% homologous to amino acids 1-131 of SEQ ID NO. 625, which also corresponds to amino acids 1-131 of SEQ ID NO. 560, and a second amino acid sequence being at least 70%, optionally at least 80%, preferably at least 85%, more preferably at least 90% and most preferably at least 95% homologous to a polypeptide sequence corresponding to amino acids 132-139 of SEQ ID NO. 560, wherein said first and second amino acid sequences are contiguous and in a sequential order.

According to preferred embodiments of the present invention, there is provided an isolated polypeptide encoding for a tail of SEQ ID NO. 560, comprising a polypeptide being at least 70%, optionally at least about 80%, preferably at least about 85%, more preferably at least about 90% and most preferably at least about 95% homologous to amino in SEQ ID NO. 560.

According to preferred embodiments of the present invention, there is provided an isolated chimeric polypeptide encoding for SEQ ID NO. 561, comprising a first amino acid sequence being at least 90% homologous to amino acids 1-131 of SEQ ID NO. 625, which also corresponds to amino acids 1-131 of SEQ ID NO. 561, and a second amino acid sequence being at least 70%, optionally at least 80%, preferably at least 85%, more preferably at least 90% and most preferably at least 95% homologous to a polypeptide sequence corresponding to amino acids 132-156 of SEQ ID NO. 561, wherein said first and second amino acid sequences are contiguous and in a sequential order.

According to preferred embodiments of the present invention, there is provided an isolated polypeptide encoding for a tail of SEQ ID NO. 561, comprising a polypeptide being at least 70%, optionally at least about 80%, preferably at least about 85%, more preferably at least about 90% and most preferably at least about 95% homologous to amino in SEQ ID NO. 561.

According to preferred embodiments of the present invention, there is provided an isolated chimeric polypeptide encoding for SEQ ID NO. 562, comprising a first amino acid sequence being at least 90% homologous to amino acids 1-81 of SEQ ID NO. 625, which also corresponds to amino acids 1-81 of SEQ ID NO. 562, and a second amino acid sequence being at least 70%, optionally at least 80%, preferably at least 85%, more preferably at least 90% and most preferably at least 95% homologous to a polypeptide sequence corresponding to amino acids 82-89 of SEQ ID NO. 562, wherein said first and second amino acid sequences are contiguous and in a sequential order.

According to preferred embodiments of the present invention, there is provided an isolated polypeptide encoding for a tail of SEQ ID NO. 562, comprising a polypeptide being at least 70%, optionally at least about 80%, preferably at least about 85%, more preferably at least about 90% and most preferably at least about 95% homologous to amino acids 82-89 in SEQ ID NO. 562.

According to preferred embodiments of the present invention, there is provided an isolated chimeric polypeptide encoding for SEQ ID NO. 563, comprising a first amino acid sequence being at least 90% homologous to amino acids 1-82 of SEQ ID NO. 625 which also corresponds to amino acids 1-82 of SEQ ID NO. 563.

According to preferred embodiments of the present invention, there is provided an isolated chimeric polypeptide encoding for SEQ ID NO. 552, comprising a first amino acid sequence being at least 70%, optionally at least 80%, preferably at least 85%, more preferably at least 90% and most preferably at least 95% homologous to a polypeptide sequence corresponding to amino acids 1-116 of FABH_HUMAN (SEQ ID NO:623), which also corresponds to amino acids 1-116 of SEQ ID NO. 552, and a second amino acid sequence being at least 70%, optionally at least 80%, preferably at least 85%, more preferably at least 90% and most preferably at least 95% homologous to a polypeptide sequence corresponding to amino acids 117-215 of SEQ ID NO. 552, wherein said first and second amino acid sequences are contiguous and in a sequential order.

According to preferred embodiments of the present invention, there is provided an isolated polypeptide encoding for a tail of SEQ ID NO. 552, comprising a polypeptide being at least 70%, optionally at least about 80%, preferably at least about 85%, more preferably at least about 90% and most preferably at least about 95% homologous to amino in SEQ ID NO. 552.

According to preferred embodiments of the present invention, there is provided an isolated chimeric polypeptide encoding for SEQ ID NO. 552, comprising a first amino acid sequence being at least 90% homologous to amino acids 1-116 of AAP35373 (SEQ ID NO:1392), which also corresponds to amino acids 1-116 of SEQ ID NO. 552, and a second amino acid sequence being at least 70%, optionally at least 80%, preferably at least 85%, more preferably at least 90% and most preferably at least 95% homologous to a polypeptide sequence corresponding to amino acids 117-215 of SEQ ID NO. 552, wherein said first and second amino acid sequences are contiguous and in a sequential order.

According to preferred embodiments of the present invention, there is provided an isolated polypeptide encoding for a tail of SEQ ID NO. 552, comprising a polypeptide being at least 70%, optionally at least about 80%, preferably at least about 85%, more preferably at least about 90% and most preferably at least about 95% homologous to amino in SEQ ID NO. 552.

According to preferred embodiments of the present invention, there is provided an isolated chimeric polypeptide encoding for SEQ ID NO. 553, comprising a first amino acid sequence being at least 70%, optionally at least 80%, preferably at least 85%, more preferably at least 90% and most preferably at least 95% homologous to a polypeptide sequence amino acids 1-116 of FABH_HUMAN (SEQ ID NO:623), which also corresponds to amino acids 1-116 of SEQ ID NO. 553, and a second amino acid sequence being at least 70%, optionally at least 80%, preferably at least 85%, more preferably at least 90% and most preferably at least 95% homologous to a polypeptide sequence corresponding to amino acids 117-178 of SEQ ID NO. 553, wherein said first and second amino acid sequences are contiguous and in a sequential order.

According to preferred embodiments of the present invention, there is provided an isolated polypeptide encoding for a tail of SEQ ID NO. 553, comprising a polypeptide being at least 70%, optionally at least about 80%, preferably at least about 85%, more preferably at least about 90% and most preferably at least about 95% homologous to amino in SEQ ID NO. 553.

According to preferred embodiments of the present invention, there is provided an isolated chimeric polypeptide encoding for SEQ ID NO. 553, comprising a first amino acid sequence being at least 90% homologous to amino acids 1-116 of AAP35373 (SEQ ID NO:1392), which also corresponds to amino acids 1-116 of SEQ ID NO. 553, and a second amino acid sequence being at least 70%, optionally at least 80%, preferably at least 85%, more preferably at least 90% and most preferably at least 95% homologous to a polypeptide sequence corresponding to amino acids 117-178 of SEQ ID NO. 553, wherein said first and second amino acid sequences are contiguous and in a sequential order.

According to preferred embodiments of the present invention, there is provided an isolated polypeptide encoding for a tail of SEQ ID NO. 553, comprising a polypeptide being at least 70%, optionally at least about 80%, preferably at least about 85%, more preferably at least about 90% and most preferably at least about 95% homologous to in SEQ ID NO. 553.

According to preferred embodiments of the present invention, there is provided an isolated chimeric polypeptide encoding for SEQ ID NO. 553, comprising a first amino acid sequence being at least 70%, optionally at least 80%, preferably at least 85%, more preferably at least 90% and most preferably at least 95% homologous to a polypeptide sequence corresponding to amino acids 1-116 of FABH_HUMAN (SEQ ID NO:623), which also corresponds to amino acids 1-116 of SEQ ID NO. 553, and a second amino acid sequence being at least 70%, optionally at least 80%, preferably at least 85%, more preferably at least 90% and most preferably at least 95% homologous to a polypeptide sequence corresponding to amino acids 117-178 of SEQ ID NO. 553, wherein said first and second amino acid sequences are contiguous and in a sequential order.

According to preferred embodiments of the present invention, there is provided an isolated polypeptide encoding for a tail of SEQ ID NO. 553, comprising a polypeptide being at least 70%, optionally at least about 80%, preferably at least about 85%, more preferably at least about 90% and most preferably at least about 95% homologous to amino in SEQ ID NO. 553.

According to preferred embodiments of the present invention, there is provided an isolated chimeric polypeptide encoding for SEQ ID NO. 553, comprising a first amino acid sequence being at least 90% homologous to amino acids 1-116 of AAP35373 (SEQ ID NO:1392), which also corresponds to amino acids 1-116 of SEQ ID NO. 553, and a second amino acid sequence being at least 70%, optionally at least 80%, preferably at least 85%, more preferably at least 90% and most preferably at least 95% homologous to a polypeptide sequence corresponding to amino acids 117-178 of SEQ ID NO. 553, wherein said first and second amino acid sequences are contiguous and in a sequential order.

According to preferred embodiments of the present invention, there is provided an isolated polypeptide encoding for a tail of SEQ ID NO. 553, comprising a polypeptide being at least 70%, optionally at least about 80%, preferably at least about 85%, more preferably at least about 90% and most preferably at least about 95% homologous to amino in SEQ ID NO. 553.

According to preferred embodiments of the present invention, there is provided an isolated chimeric polypeptide encoding for SEQ ID NO. 554, comprising a first amino acid sequence being at least 70%, optionally at least 80%, preferably at least 85%, more preferably at least 90% and most preferably at least 95% homologous to a polypeptide sequence corresponding to amino acids 1-116 of FABH_HUMAN (SEQ ID NO:623), which also corresponds to amino acids 1-116 of SEQ ID NO. 554, and a second amino acid sequence being at least 70%, optionally at least 80%, preferably at least 85%, more preferably at least 90% and most preferably at least 95% homologous to a polypeptide sequence corresponding to amino acids 117-126 of SEQ ID NO. 554, wherein said first and second amino acid sequences are contiguous and in a sequential order.

According to preferred embodiments of the present invention, there is provided an isolated polypeptide encoding for a tail of SEQ ID NO. 554, comprising a polypeptide being at least 70%, optionally at least about 80%, preferably at least about 85%, more preferably at least about 90% and most preferably at least about 95% homologous to amino in SEQ ID NO. 554.

According to preferred embodiments of the present invention, there is provided an isolated chimeric polypeptide encoding for SEQ ID NO. 554, comprising a first amino acid sequence being at least 90% homologous to amino acids 1-116 of AAP35373 (SEQ ID NO:1392), which also corresponds to amino acids 1-116 of SEQ ID NO. 554, and a second amino acid sequence being at least 70%, optionally at least 80%, preferably at least 85%, more preferably at least 90% and most preferably at least 95% homologous to a polypeptide sequence corresponding to amino acids 117-126 of SEQ ID NO. 554, wherein said first and second amino acid sequences are contiguous and in a sequential order.

According to preferred embodiments of the present invention, there is provided an isolated polypeptide encoding for a tail of SEQ ID NO. 554, comprising a polypeptide being at least 70%, optionally at least about 80%, preferably at least about 85%, more preferably at least about 90% and most preferably at least about 95% homologous to amino in SEQ ID NO. 554.

According to preferred embodiments of the present invention, there is provided an isolated chimeric polypeptide encoding for SEQ ID NO. 555, comprising a first amino acid sequence being at least 90% homologous to amino acids 1-24 of FABH_HUMAN (SEQ ID NO:623), which also corresponds to amino acids 1-24 of SEQ ID NO. 555, second amino acid sequence being at least 70%, optionally at least 80%, preferably at least 85%, more preferably at least 90% and most preferably at least 95% homologous to a polypeptide sequence corresponding to amino acids 25-35 of SEQ ID NO. 555, and a third amino acid sequence being at least 90% homologous to amino acids 25-133 of FABH_HUMAN (SEQ ID NO:623), which also corresponds to amino acids 36-144 of SEQ ID NO. 555, wherein said first, second, third and fourth amino acid sequences are contiguous and in a sequential order.

According to preferred embodiments of the present invention, there is provided an isolated polypeptide encoding for an edge portion of SEQ ID NO. 555, comprising an amino acid sequence being at least 70%, optionally at least about 80%, preferably at least about 85%, more preferably at least about 90% and most preferably at least about 95% homologous to the sequence encoding for amino acids 25-35 corresponding to SEQ ID NO. 555.

According to preferred embodiments of the present invention, there is provided an isolated chimeric polypeptide encoding for SEQ ID NO. 555, comprising a first amino acid sequence being at least 90% homologous to amino acids 1-24 of AAP35373 (SEQ ID NO:1392), which also corresponds to amino acids 1-24 of SEQ ID NO. 555, second amino acid sequence being at least 70%, optionally at least 80%, preferably at least 85%, more preferably at least 90% and most preferably at least 95% homologous to a polypeptide sequence corresponding to amino acids 25-35 of SEQ ID NO. 555, and a third amino acid sequence being at least 90% homologous to GVGFATRQVASMTKPTTIIEKNGDILTLKTHSTFKNTEISFKLGVEFDETTADDRKVKSIVTLDGGKLVHLQK WDGQETTLVRELIDGKLILTLTHGTAVCTRTYEKEA corresponding to amino acids 25-133 of AAP35373 (SEQ ID NO:1392), which also corresponds to amino acids 36-144 of SEQ ID NO. 555, wherein said first, second and third amino acid sequences are contiguous and in a sequential order.

According to preferred embodiments of the present invention, there is provided an isolated polypeptide encoding for an edge portion of SEQ ID NO. 555, comprising an amino acid sequence being at least 70%, optionally at least about 80%, preferably at least about 85%, more preferably at least about 90% and most preferably at least about 95% homologous to the sequence encoding for amino acids 25-35 corresponding to SEQ ID NO. 555.

According to preferred embodiments of the present invention, there is provided an isolated chimeric polypeptide encoding for SEQ ID NO. 534, comprising a first amino acid sequence being at least 90% homologous to amino acids 1-476 of EPB2_HUMAN (SEQ ID NO:616), which also corresponds to amino acids 1-476 of SEQ ID NO. 534, and a second amino acid sequence being at least 70%, optionally at least 80%, preferably at least 85%, more preferably at least 90% and most preferably at least 95% homologous to a polypeptide sequence corresponding to amino acids 477-496 of SEQ ID NO. 534, wherein said first and second amino acid sequences are contiguous and in a sequential order.

According to preferred embodiments of the present invention, there is provided an isolated polypeptide encoding for a tail of SEQ ID NO. 534, comprising a polypeptide being at least 70%, optionally at least about 80%, preferably at least about 85%, more preferably at least about 90% and most preferably at least about 95% homologous to amino in SEQ ID NO. 534.

According to preferred embodiments of the present invention, there is provided an isolated chimeric polypeptide encoding for SEQ ID NO. 535, comprising a first amino acid sequence being at least 90% homologous to amino acids 1-270 of EPB2_HUMAN (SEQ ID NO:616), which also corresponds to amino acids 1-270 of SEQ ID NO. 535, and a second amino acid sequence being at least 70%, optionally at least 80%, preferably at least 85%, more preferably at least 90% and most preferably at least 95% homologous to a polypeptide sequence corresponding to amino acids 271-301 of SEQ ID NO. 535, wherein said first and second amino acid sequences are contiguous and in a sequential order.

According to preferred embodiments of the present invention, there is provided an isolated polypeptide encoding for a tail of SEQ ID NO. 535, comprising a polypeptide being at least 70%, optionally at least about 80%, preferably at least about 85%, more preferably at least about 90% and most preferably at least about 95% homologous to amino in SEQ ID NO. 535.

According to preferred embodiments of the present invention, there is provided an isolated chimeric polypeptide encoding for SEQ ID NO. 536, comprising a first amino acid sequence being at least 90% homologous to amino acids 1-319 of CEA6_HUMAN (SEQ ID NO:617), which also corresponds to amino acids 1-319 of SEQ ID NO. 536, and a second amino acid sequence being at least 70%, optionally at least 80%, preferably at least 85%, more preferably at least 90% and most preferably at least 95% homologous to a polypeptide sequence corresponding to amino acids 320-324 of SEQ ID NO. 536, wherein said first and second amino acid sequences are contiguous and in a sequential order.

According to preferred embodiments of the present invention, there is provided an isolated polypeptide encoding for a tail of SEQ ID NO. 536, comprising a polypeptide being at least 70%, optionally at least about 80%, preferably at least about 85%, more preferably at least about 90% and most preferably at least about 95% homologous to amino in SEQ ID NO. 536.

According to preferred embodiments of the present invention, there is provided an isolated chimeric polypeptide encoding for SEQ ID NO. 537, comprising a first amino acid sequence being at least 90% homologous to amino acids 1-234 of CEA6_HUMAN (SEQ ID NO:617), which also corresponds to amino acids 1-234 of SEQ ID NO. 537, and a second amino acid sequence being at least 70%, optionally at least 80%, preferably at least 85%, more preferably at least 90% and most preferably at least 95% homologous to a polypeptide sequence corresponding to amino acids 235-256 of SEQ ID NO. 537, wherein said first and second amino acid sequences are contiguous and in a sequential order.

According to preferred embodiments of the present invention, there is provided an isolated polypeptide encoding for a tail of SEQ ID NO. 537, comprising a polypeptide being at least 70%, optionally at least about 80%, preferably at least about 85%, more preferably at least about 90% and most preferably at least about 95% homologous to amino in SEQ ID NO. 537.

According to preferred embodiments of the present invention, there is provided an isolated chimeric polypeptide encoding for SEQ ID NO. 537, comprising a first amino acid sequence being at least 90% homologous to amino acids 1-234 of Q13774 (SEQ ID NO:1382), which also corresponds to amino acids 1-234 of SEQ ID NO. 537, and a second amino acid sequence being at least 70%, optionally at least 80%, preferably at least 85%, more preferably at least 90% and most preferably at least 95% homologous to a polypeptide sequence corresponding to amino acids 235-256 of SEQ ID NO. 537, wherein said first and second amino acid sequences are contiguous and in a sequential order.

According to preferred embodiments of the present invention, there is provided an isolated polypeptide encoding for a tail of SEQ ID NO. 537, comprising a polypeptide being at least 70%, optionally at least about 80%, preferably at least about 85%, more preferably at least about 90% and most preferably at least about 95% homologous to in SEQ ID NO. 537.

According to preferred embodiments of the present invention, there is provided an isolated chimeric polypeptide encoding for SEQ ID NO. 538, comprising a first amino acid sequence being at least 90% homologous to amino acids 1-320 of CEA6_HUMAN (SEQ ID NO:617), which also corresponds to amino acids 1-320 of SEQ ID NO. 538, and a second amino acid sequence being at least 70%, optionally at least 80%, preferably at least 85%, more preferably at least 90% and most preferably at least 95% homologous to a polypeptide sequence corresponding to amino acids 321-390 of SEQ ID NO. 538, wherein said first and second amino acid sequences are contiguous and in a sequential order.

According to preferred embodiments of the present invention, there is provided an isolated polypeptide encoding for a tail of SEQ ID NO. 538, comprising a polypeptide being at least 70%, optionally at least about 80%, preferably at least about 85%, more preferably at least about 90% and most preferably at least about 95% homologous to amino in SEQ ID NO. 538.

According to preferred embodiments of the present invention, there is provided an isolated chimeric polypeptide encoding for SEQ ID NO. 539, comprising a first amino acid sequence being at least 90% homologous to amino acids 1-141 of CEA6_HUMAN (SEQ ID NO:617), which also corresponds to amino acids 1-141 of SEQ ID NO. 539, and a second amino acid sequence being at least 70%, optionally at least 80%, preferably at least 85%, more preferably at least 90% and most preferably at least 95% homologous to a polypeptide sequence corresponding to amino acids 142-183 of SEQ ID NO. 539, wherein said first and second amino acid sequences are contiguous and in a sequential order.

According to preferred embodiments of the present invention, there is provided an isolated polypeptide encoding for a tail of SEQ ID NO. 539, comprising a polypeptide being at least 70%, optionally at least about 80%, preferably at least about 85%, more preferably at least about 90% and most preferably at least about 95% homologous to amino in SEQ ID NO. 539.

According to preferred embodiments of the present invention, there is provided an isolated chimeric polypeptide encoding for SEQ ID NO. 540, comprising a first amino acid sequence being at least 90% homologous to amino acids 168-180 of Q9HAP5 (SEQ ID NO:1384), which also corresponds to amino acids 1-167 of SEQ ID NO. 540, and a second amino acid sequence being at least 70%, optionally at least 80%, preferably at least 85%, more preferably at least 90% and most preferably at least 95% homologous to a polypeptide sequence corresponding to amino acids 168-180 of SEQ ID NO. 540, wherein said first and second amino acid sequences are contiguous and in a sequential order.

According to preferred embodiments of the present invention, there is provided an isolated polypeptide encoding for a tail of SEQ ID NO. 540, comprising a polypeptide being at least 70%, optionally at least about 80%, preferably at least about 85%, more preferably at least about 90% and most preferably at least about 95% homologous to amino in SEQ ID NO. 540.

According to preferred embodiments of the present invention, there is provided an isolated chimeric polypeptide encoding for SEQ ID NO. 541, comprising a first amino acid sequence being at least 90% homologous to amino acids 1-357 of Q8N441 (SEQ ID NO:1385), which also corresponds to amino acids 1-357 of SEQ ID NO. 541, second amino acid sequence being at least 70%, optionally at least 80%, preferably at least 85%, more preferably at least 90% and most preferably at least 95% homologous to a polypeptide sequence corresponding to amino acids 358-437 of SEQ ID NO. 541, and a third amino acid sequence being at least 90% homologous to amino acids 358-504 of Q8N441 (SEQ ID NO:1385), which also corresponds to amino acids 438-584 of SEQ ID NO. 541, wherein said first, second and third amino acid sequences are contiguous and in a sequential order.

According to preferred embodiments of the present invention, there is provided an isolated polypeptide encoding for an edge portion of SEQ ID NO. 541, comprising an amino acid sequence being at least 70%, optionally at least about 80%, preferably at least about 85%, more preferably at least about 90% and most preferably at least about 95% homologous to the sequence encoding for corresponding to SEQ ID NO. 541.

According to preferred embodiments of the present invention, there is provided an isolated chimeric polypeptide encoding for SEQ ID NO. 542, comprising a first amino acid sequence being at least 90% homologous to amino acids 1-269 of Q9H4D7 (SEQ ID NO:1386), which also corresponds to amino acids 1-269 of SEQ ID NO. 542, and a second amino acid sequence being at least 70%, optionally at least 80%, preferably at least 85%, more preferably at least 90% and most preferably at least 95% homologous to a polypeptide sequence corresponding to amino acids 270-490 of SEQ ID NO. 542, wherein said first and second amino acid sequences are contiguous and in a sequential order.

According to preferred embodiments of the present invention, there is provided an isolated polypeptide encoding for a tail of SEQ ID NO. 542, comprising a polypeptide being at least 70%, optionally at least about 80%, preferably at least about 85%, more preferably at least about 90% and most preferably at least about 95% homologous to amino in SEQ ID NO. 542.

According to preferred embodiments of the present invention, there is provided an isolated chimeric polypeptide encoding for SEQ ID NO. 542, comprising a first amino acid sequence being at least 90% homologous to amino acids 1-269 of Q8N441 (SEQ ID NO:1385), which also corresponds to amino acids 1-269 of SEQ ID NO. 542, and a second amino acid sequence being at least 70%, optionally at least 80%, preferably at least 85%, more preferably at least 90% and most preferably at least 95% homologous to a polypeptide sequence corresponding to amino acids 270-490 of SEQ ID NO. 542, wherein said first and second amino acid sequences are contiguous and in a sequential order.

According to preferred embodiments of the present invention, there is provided an isolated polypeptide encoding for a tail of SEQ ID NO. 542, comprising a polypeptide being at least 70%, optionally at least about 80%, preferably at least about 85%, more preferably at least about 90% and most preferably at least about 95% homologous to amino in SEQ ID NO. 542.

According to preferred embodiments of the present invention, there is provided an isolated chimeric polypeptide encoding for SEQ ID NO. 543, comprising a first amino acid sequence being at least 90% homologous to amino acids 1-81 of SZ05_HUMAN (SEQ ID NO:618), which also corresponds to amino acids 1-81 of SEQ ID NO. 543.

According to preferred embodiments of the present invention, there is provided an isolated chimeric polypeptide encoding for SEQ ID NO. 544, comprising a first amino acid sequence being at least 90% homologous to amino acids 1-74 of MI2B_HUMAN (SEQ ID NO:619), which also corresponds to amino acids 1-74 of SEQ ID NO. 544.

According to preferred embodiments of the present invention, there is provided an isolated chimeric polypeptide encoding for SEQ ID NO. 545, comprising a first amino acid sequence being at least 90% homologous to amino acids 1-103 of MI2B_HUMAN (SEQ ID NO:619), which also corresponds to amino acids 1-103 of SEQ ID NO. 545.

According to preferred embodiments of the present invention, there is provided an isolated chimeric polypeptide encoding for SEQ ID NO. 546, comprising a first amino acid sequence being at least 90% homologous to amino acids 1-61 of MI2B_HUMAN (SEQ ID NO:619), which also corresponds to amino acids 1-61 of SEQ ID NO. 546, and a second amino acid sequence being at least 70%, optionally at least 80%, preferably at least 85%, more preferably at least 90% and most preferably at least 95% homologous to a polypeptide sequence corresponding to amino acids 62-98 of SEQ ID NO. 546, wherein said first and second amino acid sequences are contiguous and in a sequential order.

According to preferred embodiments of the present invention, there is provided an isolated polypeptide encoding for a tail of SEQ ID NO. 546, comprising a polypeptide being at least 70%, optionally at least about 80%, preferably at least about 85%, more preferably at least about 90% and most preferably at least about 95% homologous to amino acids 62-98 in SEQ ID NO. 546.

According to preferred embodiments of the present invention, there is provided an isolated chimeric polypeptide encoding for SEQ ID NO. 547, comprising a first amino acid sequence being at least 70%, optionally at least 80%, preferably at least 85%, more preferably at least 90% and most preferably at least 95% homologous to a polypeptide sequence corresponding to amino acids 1-103 of SEQ ID NO. 547, and a second amino acid sequence being at least 90% homologous to amino acids 34-107 of MI2B_HUMAN (SEQ ID NO:619), which also corresponds to amino acids 104-177 of SEQ ID NO. 547, wherein said first and second amino acid sequences are contiguous and in a sequential order.

According to preferred embodiments of the present invention, there is provided an isolated polypeptide encoding for a head of SEQ ID NO. 547, comprising a polypeptide being at least 70%, optionally at least about 80%, preferably at least about 85%, more preferably at least about 90% and most preferably at least about 95% homologous to amino acids 1-103 of SEQ ID NO. 547.

According to preferred embodiments of the present invention, there is provided an isolated chimeric polypeptide encoding for SEQ ID NO. 548, comprising a first amino acid sequence being at least 70%, optionally at least 80%, preferably at least 85%, more preferably at least 90% and most preferably at least 95% homologous to a polypeptide sequence corresponding to amino acids 1-29 of SEQ ID NO. 548, and a second amino acid sequence being at least 90% homologous to amino acids 151-461 of DCOR_HUMAN (SEQ ID NO:620), which also corresponds to amino acids 30-340 of SEQ ID NO. 548, wherein said first and second amino acid sequences are contiguous and in a sequential order.

According to preferred embodiments of the present invention, there is provided an isolated polypeptide encoding for a head of SEQ ID NO. 548, comprising a polypeptide being at least 70%, optionally at least about 80%, preferably at least about 85%, more preferably at least about 90% and most preferably at least about 95% homologous to amino acids 1-29 of SEQ ID NO. 548.

According to preferred embodiments of the present invention, there is provided an isolated chimeric polypeptide encoding for SEQ ID NO. 548, comprising a first amino acid sequence being at least 70%, optionally at least 80%, preferably at least 85%, more preferably at least 90% and most preferably at least 95% homologous to a polypeptide sequence corresponding to amino acids 1-29 of SEQ ID NO. 548, and a second amino acid sequence being at least 90% homologous to amino acids 40-350 of AAA59968 (SEQ ID NO:1387), which also corresponds to amino acids 30-340 of SEQ ID NO. 548, wherein said first and second amino acid sequences are contiguous and in a sequential order.

According to preferred embodiments of the present invention, there is provided an isolated polypeptide encoding for a head of SEQ ID NO. 548, comprising a polypeptide being at least 70%, optionally at least about 80%, preferably at least about 85%, more preferably at least about 90% and most preferably at least about 95% homologous to amino acids 1-29 of SEQ ID NO. 548.

According to preferred embodiments of the present invention, there is provided an isolated chimeric polypeptide encoding for SEQ ID NO. 548, comprising a first amino acid sequence being at least 70%, optionally at least 80%, preferably at least 85%, more preferably at least 90% and most preferably at least 95% homologous to a polypeptide sequence corresponding to amino acids 1-29 of SEQ ID NO. 548, and a second amino acid sequence being at least 90% homologous to amino acids 86-396 of AAH14562 (SEQ ID NO:1388), which also corresponds to amino acids 30-340 of SEQ ID NO. 548, wherein said first and second amino acid sequences are contiguous and in a sequential order.

According to preferred embodiments of the present invention, there is provided an isolated polypeptide encoding for a head of SEQ ID NO. 548, comprising a polypeptide being at least 70%, optionally at least about 80%, preferably at least about 85%, more preferably at least about 90% and most preferably at least about 95% homologous to amino acids 1-29 of SEQ ID NO. 548.

According to preferred embodiments of the present invention, there is provided an isolated chimeric polypeptide encoding for SEQ ID NO. 549, comprising a first amino acid sequence being at least 70%, optionally at least 80%, preferably at least 85%, more preferably at least 90% and most preferably at least 95% homologous to a polypeptide sequence corresponding to amino acids 1-44 of SEQ ID NO. 549, second amino acid sequence being at least 90% homologous to amino acids 74-191 of Q9NWT9 (SEQ ID NO:1389), which also corresponds to amino acids 45-162 of SEQ ID NO. 549, and a third amino acid sequence being at least 70%, optionally at least 80%, preferably at least 85%, more preferably at least 90% and most preferably at least 95% homologous to a polypeptide sequence corresponding to amino acids 163-238 of SEQ ID NO. 549, wherein said first, second and third amino acid sequences are contiguous and in a sequential order.

According to preferred embodiments of the present invention, there is provided an isolated polypeptide encoding for a head of SEQ ID NO. 549, comprising a polypeptide being at least 70%, optionally at least about 80%, preferably at least about 85%, more preferably at least about 90% and most preferably at least about 95% homologous to amino acids 1-44 of SEQ ID NO. 549.

According to preferred embodiments of the present invention, there is provided an isolated polypeptide encoding for a tail of SEQ ID NO. 549, comprising a polypeptide being at least 70%, optionally at least about 80%, preferably at least about 85%, more preferably at least about 90% and most preferably at least about 95% homologous to amino in SEQ ID NO. 549.

According to preferred embodiments of the present invention, there is provided an isolated chimeric polypeptide encoding for SEQ ID NO. 549, comprising a first amino acid sequence being at least 70%, optionally at least 80%, preferably at least 85%, more preferably at least 90% and most preferably at least 95% homologous to a polypeptide sequence corresponding to amino acids 1-44 of SEQ ID NO. 549, and a second amino acid sequence being at least 90% homologous to amino acids 21-214 of TESC_HUMAN (SEQ ID NO:621), which also corresponds to amino acids 45-238 of SEQ ID NO. 549, wherein said first and second amino acid sequences are contiguous and in a sequential order.

According to preferred embodiments of the present invention, there is provided an isolated polypeptide encoding for a head of SEQ ID NO. 549, comprising a polypeptide being at least 70%, optionally at least about 80%, preferably at least about 85%, more preferably at least about 90% and most preferably at least about 95% homologous to amino acids 1-44 of SEQ ID NO. 549.

According to preferred embodiments of the present invention, there is provided an isolated chimeric polypeptide encoding for SEQ ID NO. 550, comprising a first amino acid sequence being at least 70%, optionally at least 80%, preferably at least 85%, more preferably at least 90% and most preferably at least 95% homologous to a polypeptide sequence corresponding to amino acids 1-130 of SEQ ID NO. 550, and a second amino acid sequence being at least 90% homologous to amino acids 1-172 of Q96C98 (SEQ ID NO:1390), which also corresponds to amino acids 131-302 of SEQ ID NO. 550, wherein said first and second amino acid sequences are contiguous and in a sequential order.

According to preferred embodiments of the present invention, there is provided an isolated polypeptide encoding for a head of SEQ ID NO. 550, comprising a polypeptide being at least 70%, optionally at least about 80%, preferably at least about 85%, more preferably at least about 90% and most preferably at least about 95% homologous to amino acids 1-130 of SEQ ID NO. 550.

According to preferred embodiments of the present invention, there is provided an isolated chimeric polypeptide encoding for SEQ ID NO. 550, comprising a first amino acid sequence being at least 70%, optionally at least 80%, preferably at least 85%, more preferably at least 90% and most preferably at least 95% homologous to a polypeptide sequence corresponding to amino acids 1-74 of SEQ ID NO. 550, and a second amino acid sequence being at least 90% homologous to amino acids 53-280 of Q9BVA2 (SEQ ID NO:1391), which also corresponds to amino acids 75-302 of SEQ ID NO. 550, wherein said first and second amino acid sequences are contiguous and in a sequential order.

According to preferred embodiments of the present invention, there is provided an isolated polypeptide encoding for a head of SEQ ID NO. 550, comprising a polypeptide being at least 70%, optionally at least about 80%, preferably at least about 85%, more preferably at least about 90% and most preferably at least about 95% homologous to amino acids 1-74 of SEQ ID NO. 550.

According to preferred embodiments of the present invention, there is provided an isolated chimeric polypeptide encoding for SEQ ID NO. 551, comprising a first amino acid sequence being at least 70%, optionally at least 80%, preferably at least 85%, more preferably at least 90% and most preferably at least 95% homologous to a polypeptide sequence corresponding to amino acids 1-34 of SEQ ID NO. 551, and a second amino acid sequence being at least 90% homologous to corresponding to amino acids 60-172 of Q96C98 (SEQ ID NO:1390), which also corresponds to amino acids 35-147 of SEQ ID NO. 551, wherein said first and second amino acid sequences are contiguous and in a sequential order.

According to preferred embodiments of the present invention, there is provided an isolated polypeptide encoding for a head of SEQ ID NO. 551 comprising a polypeptide being at least 70%, optionally at least about 80%, preferably at least about 85%, more preferably at least about 90% and most preferably at least about 95% homologous to amino acids 1-34 of SEQ ID NO. 551.

According to preferred embodiments of the present invention, there is provided an isolated chimeric polypeptide encoding for SEQ ID NO. 551, comprising a first amino acid sequence being at least 70%, optionally at least 80%, preferably at least 85%, more preferably at least 90% and most preferably at least 95% homologous to a polypeptide sequence corresponding to amino acids 1-34 of SEQ ID NO. 551, and a second amino acid sequence being at least 90% homologous to corresponding to amino acids 168-280 of Q9BVA2 (SEQ ID NO:1391), which also corresponds to amino acids 35-147 of SEQ ID NO. 551, wherein said first and second amino acid sequences are contiguous and in a sequential order.

According to preferred embodiments of the present invention, there is provided an isolated polypeptide encoding for a head of SEQ ID NO. 551, comprising a polypeptide being at least 70%, optionally at least about 80%, preferably at least about 85%, more preferably at least about 90% and most preferably at least about 95% homologous to amino acids 1-34 of SEQ ID NO. 551.

According to preferred embodiments of the present invention, there is provided an isolated polypeptide encoding for a head of SEQ ID NO. 548, comprising a polypeptide being at least 70%, optionally at least about 80%, preferably at least about 85%, more preferably at least about 90% and most preferably at least about 95% homologous to amino acids 1-29 of SEQ ID NO. 548.

According to preferred embodiments of the present invention, there is provided an isolated chimeric polypeptide encoding for SEQ ID NO. 556, comprising a first amino acid sequence being at least 90% homologous to amino acids 1-441 of SMO2_HUMAN (SEQ ID NO:624), which also corresponds to amino acids 1-441 of SEQ ID NO. 556, and a second amino acid sequence being at least 70%, optionally at least 80%, preferably at least 85%, more preferably at least 90% and most preferably at least 95% homologous to a polypeptide sequence corresponding to amino acids 442-464 of SEQ ID NO. 556, wherein said first and second amino acid sequences are contiguous and in a sequential order.

According to preferred embodiments of the present invention, there is provided an An isolated polypeptide encoding for a tail of SEQ ID NO. 556, comprising a polypeptide being at least 70%, optionally at least about 80%, preferably at least about 85%, more preferably at least about 90% and most preferably at least about 95% homologous to amino in SEQ ID NO. 556.

According to preferred embodiments of the present invention, there is provided an isolated chimeric polypeptide encoding for SEQ ID NO. 557, comprising a first amino acid sequence being at least 90% homologous to amino acids 1-428 of SMO2_HUMAN (SEQ ID NO:624), which also corresponds to amino acids 1-428 of SEQ ID NO. 557, and a second amino acid sequence being at least 70%, optionally at least 80%, preferably at least 85%, more preferably at least 90% and most preferably at least 95% homologous to a polypeptide sequence corresponding to amino acids 429-434 of SEQ ID NO. 557, wherein said first and second amino acid sequences are contiguous and in a sequential order.

According to preferred embodiments of the present invention, there is provided an isolated polypeptide encoding for a tail of SEQ ID NO. 557, comprising a polypeptide being at least 70%, optionally at least about 80%, preferably at least about 85%, more preferably at least about 90% and most preferably at least about 95% homologous to amino in SEQ ID NO. 557.

According to preferred embodiments of the present invention, there is provided an isolated chimeric polypeptide encoding for SEQ ID NO. 558, comprising a first amino acid sequence being at least 90% homologous to amino acids 1-441 of SMO2_HUMAN (SEQ ID NO:624), which also corresponds to amino acids 1-441 of SEQ ID NO. 558, and a second amino acid sequence being at least 70%, optionally at least 80%, preferably at least 85%, more preferably at least 90% and most preferably at least 95% homologous to a polypeptide sequence corresponding to amino acids 442-454 of SEQ ID NO. 558, wherein said first and second amino acid sequences are contiguous and in a sequential order.

According to preferred embodiments of the present invention, there is provided an isolated polypeptide encoding for a tail of SEQ ID NO. 558, comprising a polypeptide being at least 70%, optionally at least about 80%, preferably at least about 85%, more preferably at least about 90% and most preferably at least about 95% homologous amino in SEQ ID NO. 558.

According to preferred embodiments of the present invention, there is provided an isolated chimeric polypeptide encoding for SEQ ID NO. 559, comprising a first amino acid sequence being at least 90% homologous to amino acids 1-170 of SMO2_HUMAN (SEQ ID NO:624), which also corresponds to amino acids 1-170 of SEQ ID NO. 559, and a second amino acid sequence being at least 90% homologous to amino acids 188-446 of SMO2_HUMAN (SEQ ID NO:624), which also corresponds to amino acids 171-429 of SEQ ID NO. 559, wherein said first and second amino acid sequences are contiguous and in a sequential order.

According to preferred embodiments of the present invention, there is provided an isolated chimeric polypeptide encoding for an edge portion of SEQ ID NO. 559, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise TD, having a structure as follows: a sequence starting from any of amino acid numbers 170−x to 170; and ending at any of amino acid numbers 171+((n−2)−x), in which x varies from 0 to n−2.

According to preferred embodiments of the present invention, there is provided an antibody capable of specifically binding to an epitope of an amino acid sequence from clusters of M85491, T10888, H14624, H53626, HSENA78, HUMGROG5, HUMODCA, R00299, Z19178, S67314, Z44808, Z25299, HUMF5A, HUMANK, Z39818, HUMCA1XIA, HSS100PCB, HUMPHOSLIP, D11853, R11723, M77903 and HSKITCR. Optionally said amino acid sequence corresponds to a bridge, edge portion, tail, head or insertion.

Optionally the antibody is capable of differentiating between a splice variant having said epitope and a corresponding known protein.

According to preferred embodiments of the present invention, there is provided a kit for detecting colon cancer, comprising a kit detecting overexpression of a splice variant from clusters of M85491, T10888, H14624, H53626, HSENA78, HUMGROG5, HUMODCA, R00299, Z19178, S67314, Z44808, Z25299, HUMF5A, HUMANK, Z39818, HUMCA1XIA, HSS100PCB, HUMPHOSLIP, D11853, R11723, M77903 and HSKITCR.

Optionally the kit comprises a NAT-based technology.

Optionally the kit further comprises at least one primer pair capable of selectively hybridizing to a nucleic acid sequence.

Optionally the kit further comprises at least one oligonucleotide capable of selectively hybridizing to a nucleic acid sequence.

Optionally the kit comprises an antibody.

Optionally the kit further comprises at least one reagent for performing an ELISA or a Western blot.

According to preferred embodiments of the present invention, there is provided an method for detecting colon cancer, comprising detecting overexpression of a splice variant from clusters of M85491, T10888, H14624, H53626, HSENA78, HUMGROG5, HUMODCA, R00299, Z19178, S67314, Z44808, Z25299, HUMF5A, HUMANK, Z39818, HUMCA1XIA, HSS100PCB, HUMPHOSLIP, D11853, R11723, M77903 and HSKITCR.

Optionally detecting overexpression is performed with a NAT-based technology.

Optionally said detecting overexpression is performed with an immunoassay.

Optionally the immunoassay comprises an antibody.

According to preferred embodiments of the present invention, there is provided a biomarker capable of detecting colon cancer, comprising nucleic acid sequences or a fragment thereof, or amino acid sequences or a fragment thereof from clusters of M85491, T10888, H14624, H53626, HSENA78, HUMGROG5, HUMODCA, R00299, Z19178, S67314, Z44808, Z25299, HUMF5A, HUMANK, Z39818, HUMCA1XIA, HSS100PCB, HUMPHOSLIP, D11853, R11723, M77903 and HSKITCR.

According to preferred embodiments of the present invention, there is provided a method for screening for colon cancer, comprising detecting colon cancer cells with a biomarker or an antibody or a method or assay.

According to preferred embodiments of the present invention, there is provided a method for diagnosing colon cancer, comprising detecting colon cancer cells with a biomarker or an antibody or a method or assay.

According to preferred embodiments of the present invention, there is provided a method for monitoring disease progression of colon cancer, comprising detecting colon cancer cells with a biomarker or an antibody or a method or assay.

According to preferred embodiments of the present invention, there is provided a method of selecting a therapy for colon cancer, comprising detecting colon cancer cells with a biomarker or an antibody or a method or assay and selecting a therapy according to said detection.

According to preferred embodiments of the present invention, there is provided an isolated polynucleotide comprising a nucleic acid sequence in the table below and/or:

Transcript Name

AA583399_PEA_1_T0 (SEQ ID NO: 643)
AA583399_PEA_1_T1 (SEQ ID NO: 644)
AA583399_PEA_1_T2 (SEQ ID NO: 645)
AA583399_PEA_1_T3 (SEQ ID NO: 646)
AA583399_PEA_1_T4 (SEQ ID NO: 647)
AA583399_PEA_1_T5 (SEQ ID NO: 648)
AA583399_PEA_1_T6 (SEQ ID NO: 649)
AA583399_PEA_1_T7 (SEQ ID NO: 650)
AA583399_PEA_1_T8 (SEQ ID NO: 651)
AA583399_PEA_1_T9 (SEQ ID NO: 652)
AA583399_PEA_1_T10 (SEQ ID NO: 653)
AA583399_PEA_1_T11 (SEQ ID NO: 654)
AA583399_PEA_1_T12 (SEQ ID NO: 655)
AA583399_PEA_1_T15 (SEQ ID NO: 656)
AA583399_PEA_1_T16 (SEQ ID NO: 657)
AA583399_PEA_1_T17 (SEQ ID NO: 658)

a nucleic acid sequence comprising a sequence selected from the table below:

Segment Name

AA583399_PEA_1_node_0 (SEQ ID NO: 659)
AA583399_PEA_1_node_3 (SEQ ID NO: 660)
AA583399_PEA_1_node_9 (SEQ ID NO: 661)
AA583399_PEA_1_node_10 (SEQ ID NO: 662)
AA583399_PEA_1_node_12 (SEQ ID NO: 663)
AA583399_PEA_1_node_14 (SEQ ID NO: 664)
AA583399_PEA_1_node_21 (SEQ ID NO: 665)
AA583399_PEA_1_node_24 (SEQ ID NO: 666)
AA583399_PEA_1_node_25 (SEQ ID NO: 667)
AA583399_PEA_1_node_29 (SEQ ID NO: 668)
AA583399_PEA_1_node_1 (SEQ ID NO: 669)
AA583399_PEA_1_node_2 (SEQ ID NO: 670)
AA583399_PEA_1_node_4 (SEQ ID NO: 671)
AA583399_PEA_1_node_5 (SEQ ID NO: 672)
AA583399_PEA_1_node_6 (SEQ ID NO: 673)
AA583399_PEA_1_node_7 (SEQ ID NO: 674)
AA583399_PEA_1_node_8 (SEQ ID NO: 675)
AA583399_PEA_1_node_11 (SEQ ID NO: 676)
AA583399_PEA_1_node_19 (SEQ ID NO: 677)
AA583399_PEA_1_node_27 (SEQ ID NO: 678)

According to preferred embodiments of the present invention, there is provided an isolated polypeptide comprising an amino acid sequence in the table below:

Protein Name

AA583399_PEA_1_P3 (SEQ ID NO: 683)
AA583399_PEA_1_P2 (SEQ ID NO: 684)
AA583399_PEA_1_P4 (SEQ ID NO: 685)
AA583399_PEA_1_P5 (SEQ ID NO: 686)
AA583399_PEA_1_P6 (SEQ ID NO: 687)
AA583399_PEA_1_P8 (SEQ ID NO: 688)
AA583399_PEA_1_P10 (SEQ ID NO: 689)
AA583399_PEA_1_P11 (SEQ ID NO: 690)
AA583399_PEA_1_P12 (SEQ ID NO: 691)
AA583399_PEA_1_P14 (SEQ ID NO: 692)

According to preferred embodiments of the present invention, there is provided an isolated polynucleotide comprising a nucleic acid sequence in the table below and/or:

Transcript Name

AI684092_PEA_1_T2 (SEQ ID NO: 693)
AI684092_PEA_1_T3 (SEQ ID NO: 694)

a nucleic acid sequence comprising a sequence in the table below:

Segment Name

AI684092_PEA_1_node_0 (SEQ ID NO: 695)
AI684092_PEA_1_node_2 (SEQ ID NO: 696)
AI684092_PEA_1_node_4 (SEQ ID NO: 697)
AI684092_PEA_1_node_5 (SEQ ID NO: 698)
AI684092_PEA_1_node_6 (SEQ ID NO: 699)
AI684092_PEA_1_node_7 (SEQ ID NO: 700)
AI684092_PEA_1_node_8 (SEQ ID NO: 701)
AI684092_PEA_1_node_9 (SEQ ID NO: 702)

According to preferred embodiments of the present invention, there is provided an isolated polypeptide comprising an amino acid sequence in the table below:

Protein Name

AI684092_PEA_1_P1 (SEQ ID NO: 703)
AI684092_PEA_1_P3 (SEQ ID NO: 704)

According to preferred embodiments of the present invention, there is provided an isolated polynucleotide comprising a nucleic acid sequence in the table below and/or:

Transcript Name

HUMCACH1A_PEA_1_T0 (SEQ ID NO: 705)
HUMCACH1A_PEA_1_T1 (SEQ ID NO: 706)
HUMCACH1A_PEA_1_T2 (SEQ ID NO: 707)
HUMCACH1A_PEA_1_T3 (SEQ ID NO: 708)
HUMCACH1A_PEA_1_T4 (SEQ ID NO: 709)
HUMCACH1A_PEA_1_T6 (SEQ ID NO: 710)
HUMCACH1A_PEA_1_T7 (SEQ ID NO: 711)
HUMCACH1A_PEA_1_T8 (SEQ ID NO: 712)
HUMCACH1A_PEA_1_T12 (SEQ ID NO: 713)
HUMCACH1A_PEA_1_T13 (SEQ ID NO: 714)
HUMCACH1A_PEA_1_T14 (SEQ ID NO: 715)
HUMCACH1A_PEA_1_T15 (SEQ ID NO: 716)
HUMCACH1A_PEA_1_T16 (SEQ ID NO: 717)
HUMCACH1A_PEA_1_T17 (SEQ ID NO: 718)
HUMCACH1A_PEA_1_T18 (SEQ ID NO: 719)
HUMCACH1A_PEA_1_T19 (SEQ ID NO: 720)
HUMCACH1A_PEA_1_T20 (SEQ ID NO: 721)
HUMCACH1A_PEA_1_T22 (SEQ ID NO: 722)

a nucleic acid sequence comprising a sequence in the table below:

Segment Name

HUMCACH1A_PEA_1_node_2 (SEQ ID NO: 723)
HUMCACH1A_PEA_1_node_5 (SEQ ID NO: 724)
HUMCACH1A_PEA_1_node_9 (SEQ ID NO: 725)
HUMCACH1A_PEA_1_node_11 (SEQ ID NO: 726)
HUMCACH1A_PEA_1_node_14 (SEQ ID NO: 727)
HUMCACH1A_PEA_1_node_16 (SEQ ID NO: 728)
HUMCACH1A_PEA_1_node_27 (SEQ ID NO: 729)
HUMCACH1A_PEA_1_node_30 (SEQ ID NO: 730)
HUMCACH1A_PEA_1_node_33 (SEQ ID NO: 731)
HUMCACH1A_PEA_1_node_41 (SEQ ID NO: 732)
HUMCACH1A_PEA_1_node_43 (SEQ ID NO: 733)
HUMCACH1A_PEA_1_node_45 (SEQ ID NO: 734)
HUMCACH1A_PEA_1_node_47 (SEQ ID NO: 735)
HUMCACH1A_PEA_1_node_55 (SEQ ID NO: 736)
HUMCACH1A_PEA_1_node_57 (SEQ ID NO: 737)
HUMCACH1A_PEA_1_node_70 (SEQ ID NO: 738)
HUMCACH1A_PEA_1_node_72 (SEQ ID NO: 739)
HUMCACH1A_PEA_1_node_74 (SEQ ID NO: 740)
HUMCACH1A_PEA_1_node_86 (SEQ ID NO: 741)
HUMCACH1A_PEA_1_node_92 (SEQ ID NO: 742)
HUMCACH1A_PEA_1_node_94 (SEQ ID NO: 743)
HUMCACH1A_PEA_1_node_103 (SEQ ID NO: 744)
HUMCACH1A_PEA_1_node_104 (SEQ ID NO: 745)
HUMCACH1A_PEA_1_node_106 (SEQ ID NO: 746)
HUMCACH1A_PEA_1_node_109 (SEQ ID NO: 747)
HUMCACH1A_PEA_1_node_113 (SEQ ID NO: 748)
HUMCACH1A_PEA_1_node_114 (SEQ ID NO: 749)
HUMCACH1A_PEA_1_node_116 (SEQ ID NO: 750)
HUMCACH1A_PEA_1_node_119 (SEQ ID NO: 751)
HUMCACH1A_PEA_1_node_121 (SEQ ID NO: 752)
HUMCACH1A_PEA_1_node_123 (SEQ ID NO: 753)
HUMCACH1A_PEA_1_node_125 (SEQ ID NO: 754)
HUMCACH1A_PEA_1_node_128 (SEQ ID NO: 755)
HUMCACH1A_PEA_1_node_0 (SEQ ID NO: 756)
HUMCACH1A_PEA_1_node_3 (SEQ ID NO: 757)
HUMCACH1A_PEA_1_node_7 (SEQ ID NO: 758)
HUMCACH1A_PEA_1_node_23 (SEQ ID NO: 759)
HUMCACH1A_PEA_1_node_26 (SEQ ID NO: 760)
HUMCACH1A_PEA_1_node_32 (SEQ ID NO: 761)
HUMCACH1A_PEA_1_node_35 (SEQ ID NO: 762)
HUMCACH1A_PEA_1_node_37 (SEQ ID NO: 763)
HUMCACH1A_PEA_1_node_39 (SEQ ID NO: 764)
HUMCACH1A_PEA_1_node_49 (SEQ ID NO: 765)
HUMCACH1A_PEA_1_node_51 (SEQ ID NO: 766)
HUMCACH1A_PEA_1_node_53 (SEQ ID NO: 767)
HUMCACH1A_PEA_1_node_58 (SEQ ID NO: 768)
HUMCACH1A_PEA_1_node_60 (SEQ ID NO: 769)
HUMCACH1A_PEA_1_node_62 (SEQ ID NO: 770)
HUMCACH1A_PEA_1_node_64 (SEQ ID NO: 771)
HUMCACH1A_PEA_1_node_66 (SEQ ID NO: 772)
HUMCACH1A_PEA_1_node_68 (SEQ ID NO: 773)
HUMCACH1A_PEA_1_node_76 (SEQ ID NO: 774)
HUMCACH1A_PEA_1_node_77 (SEQ ID NO: 775)
HUMCACH1A_PEA_1_node_79 (SEQ ID NO: 776)
HUMCACH1A_PEA_1_node_81 (SEQ ID NO: 777)
HUMCACH1A_PEA_1_node_84 (SEQ ID NO: 778)
HUMCACH1A_PEA_1_node_88 (SEQ ID NO: 779)
HUMCACH1A_PEA_1_node_90 (SEQ ID NO: 780)
HUMCACH1A_PEA_1_node_96 (SEQ ID NO: 781)
HUMCACH1A_PEA_1_node_98 (SEQ ID NO: 782)
HUMCACH1A_PEA_1_node_100 (SEQ ID NO: 783)
HUMCACH1A_PEA_1_node_101 (SEQ ID NO: 784)
HUMCACH1A_PEA_1_node_107 (SEQ ID NO: 785)
HUMCACH1A_PEA_1_node_111 (SEQ ID NO: 786)
HUMCACH1A_PEA_1_node_117 (SEQ ID NO: 787)
HUMCACH1A_PEA_1_node_124 (SEQ ID NO: 788)
HUMCACH1A_PEA_1_node_126 (SEQ ID NO: 789)

According to preferred embodiments of the present invention, there is provided an isolated polypeptide comprising an amino acid sequence in the table below:

Protein Name

HUMCACH1A_PEA_1_P2 (SEQ ID NO: 792)
HUMCACH1A_PEA_1_P3 (SEQ ID NO: 793)
HUMCACH1A_PEA_1_P4 (SEQ ID NO: 794)
HUMCACH1A_PEA_1_P5 (SEQ ID NO: 795)
HUMCACH1A_PEA_1_P7 (SEQ ID NO: 796)
HUMCACH1A_PEA_1_P8 (SEQ ID NO: 797)
HUMCACH1A_PEA_1_P9 (SEQ ID NO: 798)
HUMCACH1A_PEA_1_P10 (SEQ ID NO: 799)
HUMCACH1A_PEA_1_P11 (SEQ ID NO: 800)
HUMCACH1A_PEA_1_P12 (SEQ ID NO: 801)
HUMCACH1A_PEA_1_P13 (SEQ ID NO: 802)
HUMCACH1A_PEA_1_P14 (SEQ ID NO: 803)
HUMCACH1A_PEA_1_P15 (SEQ ID NO: 804)
HUMCACH1A_PEA_1_P17 (SEQ ID NO: 805)

According to preferred embodiments of the present invention, there is provided an isolated polynucleotide comprising a nucleic acid sequence in the table below and/or:

Transcript Name

HUMCEA_PEA_1_T8 (SEQ ID NO: 806)
HUMCEA_PEA_1_T9 (SEQ ID NO: 807)
HUMCEA_PEA_1_T12 (SEQ ID NO: 808)
HUMCEA_PEA_1_T14 (SEQ ID NO: 809)
HUMCEA_PEA_1_T16 (SEQ ID NO: 810)
HUMCEA_PEA_1_T20 (SEQ ID NO: 811)
HUMCEA_PEA_1_T25 (SEQ ID NO: 812)
HUMCEA_PEA_1_T26 (SEQ ID NO: 813)
HUMCEA_PEA_1_T29 (SEQ ID NO: 814)
HUMCEA_PEA_1_T30 (SEQ ID NO: 815)

a nucleic acid sequence comprising a sequence in the table below:

Segment Name

HUMCEA_PEA_1_node_0 (SEQ ID NO: 816)
HUMCEA_PEA_1_node_2 (SEQ ID NO: 817)
HUMCEA_PEA_1_node_6 (SEQ ID NO: 818)
HUMCEA_PEA_1_node_11 (SEQ ID NO: 819)
HUMCEA_PEA_1_node_12 (SEQ ID NO: 820)
HUMCEA_PEA_1_node_31 (SEQ ID NO: 821)
HUMCEA_PEA_1_node_36 (SEQ ID NO: 822)
HUMCEA_PEA_1_node_42 (SEQ ID NO: 823)
HUMCEA_PEA_1_node_43 (SEQ ID NO: 824)
HUMCEA_PEA_1_node_44 (SEQ ID NO: 825)
HUMCEA_PEA_1_node_46 (SEQ ID NO: 826)
HUMCEA_PEA_1_node_48 (SEQ ID NO: 827)
HUMCEA_PEA_1_node_63 (SEQ ID NO: 828)
HUMCEA_PEA_1_node_65 (SEQ ID NO: 829)
HUMCEA_PEA_1_node_67 (SEQ ID NO: 830)
HUMCEA_PEA_1_node_3 (SEQ ID NO: 831)
HUMCEA_PEA_1_node_7 (SEQ ID NO: 832)
HUMCEA_PEA_1_node_8 (SEQ ID NO: 833)
HUMCEA_PEA_1_node_9 (SEQ ID NO: 834)
HUMCEA_PEA_1_node_10 (SEQ ID NO: 835)
HUMCEA_PEA_1_node_15 (SEQ ID NO: 836)
HUMCEA_PEA_1_node_16 (SEQ ID NO: 837)
HUMCEA_PEA_1_node_17 (SEQ ID NO: 838)
HUMCEA_PEA_1_node_18 (SEQ ID NO: 839)
HUMCEA_PEA_1_node_19 (SEQ ID NO: 840)
HUMCEA_PEA_1_node_20 (SEQ ID NO: 841)
HUMCEA_PEA_1_node_21 (SEQ ID NO: 842)
HUMCEA_PEA_1_node_22 (SEQ ID NO: 843)
HUMCEA_PEA_1_node_23 (SEQ ID NO: 844)
HUMCEA_PEA_1_node_24 (SEQ ID NO: 845)
HUMCEA_PEA_1_node_27 (SEQ ID NO: 846)
HUMCEA_PEA_1_node_29 (SEQ ID NO: 847)
HUMCEA_PEA_1_node_30 (SEQ ID NO: 848)
HUMCEA_PEA_1_node_33 (SEQ ID NO: 849)
HUMCEA_PEA_1_node_34 (SEQ ID NO: 850)
HUMCEA_PEA_1_node_35 (SEQ ID NO: 851)
HUMCEA_PEA_1_node_45 (SEQ ID NO: 852)
HUMCEA_PEA_1_node_49 (SEQ ID NO: 853)
HUMCEA_PEA_1_node_50 (SEQ ID NO: 854)
HUMCEA_PEA_1_node_51 (SEQ ID NO: 855)
HUMCEA_PEA_1_node_56 (SEQ ID NO: 856)
HUMCEA_PEA_1_node_57 (SEQ ID NO: 857)
HUMCEA_PEA_1_node_58 (SEQ ID NO: 858)
HUMCEA_PEA_1_node_60 (SEQ ID NO: 859)
HUMCEA_PEA_1_node_61 (SEQ ID NO: 860)
HUMCEA_PEA_1_node_62 (SEQ ID NO: 861)
HUMCEA_PEA_1_node_64 (SEQ ID NO: 862)

According to preferred embodiments of the present invention, there is provided an isolated polypeptide comprising an amino acid sequence in the table below:

Protein Name

HUMCEA_PEA_1_P4 (SEQ ID NO: 864)
HUMCEA_PEA_1_P5 (SEQ ID NO: 865)
HUMCEA_PEA_1_P7 (SEQ ID NO: 866)
HUMCEA_PEA_1_P10 (SEQ ID NO: 867)
HUMCEA_PEA_1_P14 (SEQ ID NO: 868)
HUMCEA_PEA_1_P19 (SEQ ID NO: 869)
HUMCEA_PEA_1_P20 (SEQ ID NO: 870)

According to preferred embodiments of the present invention, there is provided an isolated polynucleotide comprising a nucleic acid sequence in the table below and/or:

Transcript Name

M78035_T0 (SEQ ID NO: 871)
M78035_T3 (SEQ ID NO: 872)
M78035_T4 (SEQ ID NO: 873)
M78035_T7 (SEQ ID NO: 874)
M78035_T9 (SEQ ID NO: 875)
M78035_T11 (SEQ ID NO: 876)
M78035_T17 (SEQ ID NO: 877)
M78035_T18 (SEQ ID NO: 878)
M78035_T19 (SEQ ID NO: 879)
M78035_T20 (SEQ ID NO: 880)
M78035_T27 (SEQ ID NO: 881)
M78035_T28 (SEQ ID NO: 882)

a nucleic acid sequence comprising a sequence in the table below:

Segment Name

M78035_node_4 (SEQ ID NO: 883)
M78035_node_6 (SEQ ID NO: 884)
M78035_node_10 (SEQ ID NO: 885)
M78035_node_17 (SEQ ID NO: 886)
M78035_node_18 (SEQ ID NO: 887)
M78035_node_21 (SEQ ID NO: 888)
M78035_node_25 (SEQ ID NO: 889)
M78035_node_33 (SEQ ID NO: 890)
M78035_node_55 (SEQ ID NO: 891)
M78035_node_58 (SEQ ID NO: 892)
M78035_node_60 (SEQ ID NO: 893)
M78035_node_62 (SEQ ID NO: 894)
M78035_node_63 (SEQ ID NO: 895)
M78035_node_64 (SEQ ID NO: 896)
M78035_node_65 (SEQ ID NO: 897)
M78035_node_69 (SEQ ID NO: 898)
M78035_node_71 (SEQ ID NO: 899)
M78035_node_14 (SEQ ID NO: 900)
M78035_node_15 (SEQ ID NO: 901)
M78035_node_20 (SEQ ID NO: 902)
M78035_node_24 (SEQ ID NO: 903)
M78035_node_26 (SEQ ID NO: 904)
M78035_node_28 (SEQ ID NO: 905)
M78035_node_29 (SEQ ID NO: 906)
M78035_node_30 (SEQ ID NO: 907)
M78035_node_31 (SEQ ID NO: 908)
M78035_node_34 (SEQ ID NO: 909)
M78035_node_35 (SEQ ID NO: 910)
M78035_node_37 (SEQ ID NO: 911)
M78035_node_40 (SEQ ID NO: 912)
M78035_node_48 (SEQ ID NO: 913)
M78035_node_49 (SEQ ID NO: 914)
M78035_node_50 (SEQ ID NO: 915)
M78035_node_52 (SEQ ID NO: 916)
M78035_node_53 (SEQ ID NO: 917)
M78035_node_54 (SEQ ID NO: 918)
M78035_node_56 (SEQ ID NO: 919)
M78035_node_57 (SEQ ID NO: 920)
M78035_node_59 (SEQ ID NO: 921)

According to preferred embodiments of the present invention, there is provided an isolated polypeptide comprising an amino acid sequence in the table below:

Protein Name

M78035_P2 (SEQ ID NO: 923)
M78035_P4 (SEQ ID NO: 924)
M78035_P6 (SEQ ID NO: 925)
M78035_P8 (SEQ ID NO: 926)
M78035_P18 (SEQ ID NO: 927)
M78035_P19 (SEQ ID NO: 928)

According to preferred embodiments of the present invention, there is provided an isolated polynucleotide comprising a nucleic acid sequence in the table below and/or:

Transcript Name

R30650_PEA_2_T2 (SEQ ID NO: 929)
R30650_PEA_2_T3 (SEQ ID NO: 930)
R30650_PEA_2_T6 (SEQ ID NO: 931)
R30650_PEA_2_T14 (SEQ ID NO: 932)
R30650_PEA_2_T15 (SEQ ID NO: 933)
R30650_PEA_2_T18 (SEQ ID NO: 934)
R30650_PEA_2_T21 (SEQ ID NO: 935)
R30650_PEA_2_T23 (SEQ ID NO: 936)

a nucleic acid sequence comprising a sequence in the table below:

Segment Name

R30650_PEA_2_node_0 (SEQ ID NO: 937)
R30650_PEA_2_node_1 (SEQ ID NO: 938)
R30650_PEA_2_node_3 (SEQ ID NO: 939)
R30650_PEA_2_node_5 (SEQ ID NO: 940)
R30650_PEA_2_node_9 (SEQ ID NO: 941)
R30650_PEA_2_node_11 (SEQ ID NO: 942)
R30650_PEA_2_node_14 (SEQ ID NO: 943)
R30650_PEA_2_node_20 (SEQ ID NO: 944)
R30650_PEA_2_node_22 (SEQ ID NO: 945)
R30650_PEA_2_node_24 (SEQ ID NO: 946)
R30650_PEA_2_node_26 (SEQ ID NO: 947)
R30650_PEA_2_node_32 (SEQ ID NO: 948)
R30650_PEA_2_node_34 (SEQ ID NO: 949)
R30650_PEA_2_node_36 (SEQ ID NO: 950)
R30650_PEA_2_node_37 (SEQ ID NO: 951)
R30650_PEA_2_node_39 (SEQ ID NO: 952)
R30650_PEA_2_node_41 (SEQ ID NO: 953)
R30650_PEA_2_node_42 (SEQ ID NO: 954)
R30650_PEA_2_node_44 (SEQ ID NO: 955)
R30650_PEA_2_node_46 (SEQ ID NO: 956)
R30650_PEA_2_node_50 (SEQ ID NO: 957)
R30650_PEA_2_node_56 (SEQ ID NO: 958)
R30650_PEA_2_node_60 (SEQ ID NO: 959)
R30650_PEA_2_node_63 (SEQ ID NO: 960)
R30650_PEA_2_node_67 (SEQ ID NO: 961)
R30650_PEA_2_node_70 (SEQ ID NO: 962)
R30650_PEA_2_node_72 (SEQ ID NO: 963)
R30650_PEA_2_node_73 (SEQ ID NO: 964)
R30650_PEA_2_node_75 (SEQ ID NO: 965)
R30650_PEA_2_node_79 (SEQ ID NO: 966)
R30650_PEA_2_node_86 (SEQ ID NO: 967)
R30650_PEA_2_node_87 (SEQ ID NO: 968)
R30650_PEA_2_node_89 (SEQ ID NO: 969)
R30650_PEA_2_node_93 (SEQ ID NO: 970)
R30650_PEA_2_node_8 (SEQ ID NO: 971)
R30650_PEA_2_node_17 (SEQ ID NO: 972)
R30650_PEA_2_node_28 (SEQ ID NO: 973)
R30650_PEA_2_node_31 (SEQ ID NO: 974)
R30650_PEA_2_node_48 (SEQ ID NO: 975)
R30650_PEA_2_node_53 (SEQ ID NO: 976)
R30650_PEA_2_node_58 (SEQ ID NO: 977)
R30650_PEA_2_node_68 (SEQ ID NO: 978)
R30650_PEA_2_node_77 (SEQ ID NO: 979)
R30650_PEA_2_node_82 (SEQ ID NO: 980)
R30650_PEA_2_node_85 (SEQ ID NO: 981)
R30650_PEA_2_node_88 (SEQ ID NO: 982)
R30650_PEA_2_node_90 (SEQ ID NO: 983)
R30650_PEA_2_node_91 (SEQ ID NO: 984)
R30650_PEA_2_node_92 (SEQ ID NO: 985)

According to preferred embodiments of the present invention, there is provided an isolated polypeptide comprising an amino acid sequence in the table below:

Protein Name

R30650_PEA_2_P4 (SEQ ID NO: 991)
R30650_PEA_2_P5 (SEQ ID NO: 992)
R30650_PEA_2_P8 (SEQ ID NO: 993)
R30650_PEA_2_P12 (SEQ ID NO: 994)
R30650_PEA_2_P13 (SEQ ID NO: 995)
R30650_PEA_2_P15 (SEQ ID NO: 996)
R30650_PEA_2_P17 (SEQ ID NO: 997)

According to preferred embodiments of the present invention, there is provided an isolated polynucleotide comprising a nucleic acid sequence in the table below and/or:

Transcript Name

T23657_T0 (SEQ ID NO: 998)
T23657_T1 (SEQ ID NO: 999)
T23657_T2 (SEQ ID NO: 1000)
T23657_T3 (SEQ ID NO: 1001)
T23657_T4 (SEQ ID NO: 1002)
T23657_T5 (SEQ ID NO: 1003)
T23657_T6 (SEQ ID NO: 1004)
T23657_T7 (SEQ ID NO: 1005)
T23657_T8 (SEQ ID NO: 1006)
T23657_T9 (SEQ ID NO: 1007)
T23657_T10 (SEQ ID NO: 1008)
T23657_T11 (SEQ ID NO: 1009)
T23657_T12 (SEQ ID NO: 1010)
T23657_T13 (SEQ ID NO: 1011)
T23657_T14 (SEQ ID NO: 1012)
T23657_T15 (SEQ ID NO: 1013)
T23657_T16 (SEQ ID NO: 1014)
T23657_T17 (SEQ ID NO: 1015)
T23657_T19 (SEQ ID NO: 1016)
T23657_T20 (SEQ ID NO: 1017)
T23657_T21 (SEQ ID NO: 1018)
T23657_T22 (SEQ ID NO: 1019)
T23657_T23 (SEQ ID NO: 1020)
T23657_T24 (SEQ ID NO: 1021)
T23657_T28 (SEQ ID NO: 1022)
T23657_T30 (SEQ ID NO: 1023)
T23657_T31 (SEQ ID NO: 1024)
T23657_T32 (SEQ ID NO: 1025)
T23657_T35 (SEQ ID NO: 1026)
T23657_T37 (SEQ ID NO: 1027)
T23657_T38 (SEQ ID NO: 1028)

a nucleic acid sequence comprising a sequence in the table below:

Segment Name

T23657_node_2 (SEQ ID NO: 1029)
T23657_node_3 (SEQ ID NO: 1030)
T23657_node_8 (SEQ ID NO: 1031)
T23657_node_16 (SEQ ID NO: 1032)
T23657_node_18 (SEQ ID NO: 1033)
T23657_node_23 (SEQ ID NO: 1034)
T23657_node_24 (SEQ ID NO: 1035)
T23657_node_27 (SEQ ID NO: 1036)
T23657_node_29 (SEQ ID NO: 1037)
T23657_node_34 (SEQ ID NO: 1038)
T23657_node_37 (SEQ ID NO: 1039)
T23657_node_38 (SEQ ID NO: 1040)
T23657_node_39 (SEQ ID NO: 1041)
T23657_node_40 (SEQ ID NO: 1042)
T23657_node_45 (SEQ ID NO: 1043)
T23657_node_46 (SEQ ID NO: 1044)
T23657_node_49 (SEQ ID NO: 1045)
T23657_node_0 (SEQ ID NO: 1046)
T23657_node_4 (SEQ ID NO: 1047)
T23657_node_6 (SEQ ID NO: 1048)
T23657_node_11 (SEQ ID NO: 1049)
T23657_node_20 (SEQ ID NO: 1050)
T23657_node_22 (SEQ ID NO: 1051)
T23657_node_25 (SEQ ID NO: 1052)
T23657_node_26 (SEQ ID NO: 1053)
T23657_node_28 (SEQ ID NO: 1054)
T23657_node_30 (SEQ ID NO: 1055)
T23657_node_31 (SEQ ID NO: 1056)
T23657_node_32 (SEQ ID NO: 1057)
T23657_node_41 (SEQ ID NO: 1058)
T23657_node_42 (SEQ ID NO: 1059)
T23657_node_43 (SEQ ID NO: 1060)
T23657_node_44 (SEQ ID NO: 1061)

According to preferred embodiments of the present invention, there is provided an isolated polypeptide comprising an amino acid sequence in the table below:

Protein Name

T23657_P1 (SEQ ID NO: 1063)
T23657_P2 (SEQ ID NO: 1064)
T23657_P3 (SEQ ID NO: 1065)
T23657_P4 (SEQ ID NO: 1066)
T23657_P5 (SEQ ID NO: 1067)
T23657_P6 (SEQ ID NO: 1068)
T23657_P7 (SEQ ID NO: 1069)
T23657_P8 (SEQ ID NO: 1070)
T23657_P9 (SEQ ID NO: 1071)
T23657_P10 (SEQ ID NO: 1072)
T23657_P11 (SEQ ID NO: 1073)
T23657_P12 (SEQ ID NO: 1074)
T23657_P16 (SEQ ID NO: 1075)
T23657_P17 (SEQ ID NO: 1076)
T23657_P19 (SEQ ID NO: 1077)
T23657_P21 (SEQ ID NO: 1078)
T23657_P22 (SEQ ID NO: 1079)
T23657_P23 (SEQ ID NO: 1080)

According to preferred embodiments of the present invention, there is provided an isolated polynucleotide comprising a nucleic acid sequence in the table below and/or:

Transcript Name

T51958_PEA_1_T4 (SEQ ID NO: 1081)
T51958_PEA_1_T5 (SEQ ID NO: 1082)
T51958_PEA_1_T6 (SEQ ID NO: 1083)
T51958_PEA_1_T8 (SEQ ID NO: 1084)
T51958_PEA_1_T12 (SEQ ID NO: 1085)
T51958_PEA_1_T16 (SEQ ID NO: 1086)
T51958_PEA_1_T33 (SEQ ID NO: 1087)
T51958_PEA_1_T35 (SEQ ID NO: 1088)
T51958_PEA_1_T37 (SEQ ID NO: 1089)
T51958_PEA_1_T39 (SEQ ID NO: 1090)
T51958_PEA_1_T40 (SEQ ID NO: 1091)
T51958_PEA_1_T41 (SEQ ID NO: 1092)

a nucleic acid sequence comprising a sequence in the table below:

Segment Name

T51958_PEA_1_node_0 (SEQ ID NO: 1093)
T51958_PEA_1_node_7 (SEQ ID NO: 1094)
T51958_PEA_1_node_8 (SEQ ID NO: 1095)
T51958_PEA_1_node_9 (SEQ ID NO: 1096)
T51958_PEA_1_node_14 (SEQ ID NO: 1097)
T51958_PEA_1_node_16 (SEQ ID NO: 1098)
T51958_PEA_1_node_18 (SEQ ID NO: 1099)
T51958_PEA_1_node_21 (SEQ ID NO: 1100)
T51958_PEA_1_node_22 (SEQ ID NO: 1101)
T51958_PEA_1_node_24 (SEQ ID NO: 1102)
T51958_PEA_1_node_27 (SEQ ID NO: 1103)
T51958_PEA_1_node_29 (SEQ ID NO: 1104)
T51958_PEA_1_node_33 (SEQ ID NO: 1105)
T51958_PEA_1_node_40 (SEQ ID NO: 1106)
T51958_PEA_1_node_41 (SEQ ID NO: 1107)
T51958_PEA_1_node_46 (SEQ ID NO: 1108)
T51958_PEA_1_node_51 (SEQ ID NO: 1109)
T51958_PEA_1_node_55 (SEQ ID NO: 1110)
T51958_PEA_1_node_67 (SEQ ID NO: 1111)
T51958_PEA_1_node_70 (SEQ ID NO: 1112)
T51958_PEA_1_node_74 (SEQ ID NO: 1113)
T51958_PEA_1_node_78 (SEQ ID NO: 1114)
T51958_PEA_1_node_11 (SEQ ID NO: 1115)
T51958_PEA_1_node_15 (SEQ ID NO: 1116)
T51958_PEA_1_node_20 (SEQ ID NO: 1117)
T51958_PEA_1_node_26 (SEQ ID NO: 1118)
T51958_PEA_1_node_35 (SEQ ID NO: 1119)
T51958_PEA_1_node_36 (SEQ ID NO: 1120)
T51958_PEA_1_node_38 (SEQ ID NO: 1121)
T51958_PEA_1_node_39 (SEQ ID NO: 1122)
T51958_PEA_1_node_42 (SEQ ID NO: 1123)
T51958_PEA_1_node_43 (SEQ ID NO: 1124)
T51958_PEA_1_node_44 (SEQ ID NO: 1125)
T51958_PEA_1_node_45 (SEQ ID NO: 1126)
T51958_PEA_1_node_47 (SEQ ID NO: 1127)
T51958_PEA_1_node_48 (SEQ ID NO: 1128)
T51958_PEA_1_node_49 (SEQ ID NO: 1129)
T51958_PEA_1_node_50 (SEQ ID NO: 1130)
T51958_PEA_1_node_54 (SEQ ID NO: 1131)
T51958_PEA_1_node_61 (SEQ ID NO: 1132)
T51958_PEA_1_node_71 (SEQ ID NO: 1133)
T51958_PEA_1_node_72 (SEQ ID NO: 1134)
T51958_PEA_1_node_75 (SEQ ID NO: 1135)
T51958_PEA_1_node_76 (SEQ ID NO: 1136)
T51958_PEA_1_node_77 (SEQ ID NO: 1137)
T51958_PEA_1_node_80 (SEQ ID NO: 1138)
T51958_PEA_1_node_82 (SEQ ID NO: 1139)
T51958_PEA_1_node_84 (SEQ ID NO: 1140)

According to preferred embodiments of the present invention, there is provided an isolated polypeptide comprising an amino acid sequence in the table below:

Protein Name

T51958_PEA_1_P5 (SEQ ID NO: 1151)
T51958_PEA_1_P6 (SEQ ID NO: 1152)
T51958_PEA_1_P28 (SEQ ID NO: 1153)
T51958_PEA_1_P30 (SEQ ID NO: 1154)
T51958_PEA_1_P34 (SEQ ID NO: 1155)
T51958_PEA_1_P35 (SEQ ID NO: 1156)

According to preferred embodiments of the present invention, there is provided an isolated polynucleotide comprising a nucleic acid sequence in the table below and/or:

Transcript Name

Z17877_PEA_1_T0 (SEQ ID NO: 1157)
Z17877_PEA_1_T2 (SEQ ID NO: 1158)
Z17877_PEA_1_T3 (SEQ ID NO: 1159)
Z17877_PEA_1_T4 (SEQ ID NO: 1160)
Z17877_PEA_1_T6 (SEQ ID NO: 1161)
Z17877_PEA_1_T7 (SEQ ID NO: 1162)
Z17877_PEA_1_T8 (SEQ ID NO: 1163)
Z17877_PEA_1_T11 (SEQ ID NO: 1164)
Z17877_PEA_1_T12 (SEQ ID NO: 1165)

a nucleic acid sequence comprising a sequence in the table below:

Segment Name

Z17877_PEA_1_node_0 (SEQ ID NO: 1166)
Z17877_PEA_1_node_3 (SEQ ID NO: 1167)
Z17877_PEA_1_node_8 (SEQ ID NO: 1168)
Z17877_PEA_1_node_9 (SEQ ID NO: 1169)
Z17877_PEA_1_node_10 (SEQ ID NO: 1170)
Z17877_PEA_1_node_11 (SEQ ID NO: 1171)
Z17877_PEA_1_node_13 (SEQ ID NO: 1172)
Z17877_PEA_1_node_15 (SEQ ID NO: 1173)
Z17877_PEA_1_node_16 (SEQ ID NO: 1174)
Z17877_PEA_1_node_18 (SEQ ID NO: 1175)
Z17877_PEA_1_node_1 (SEQ ID NO: 1176)
Z17877_PEA_1_node_2 (SEQ ID NO: 1177)
Z17877_PEA_1_node_4 (SEQ ID NO: 1178)
Z17877_PEA_1_node_5 (SEQ ID NO: 1179)
Z17877_PEA_1_node_6 (SEQ ID NO: 1180)
Z17877_PEA_1_node_14 (SEQ ID NO: 1181)
Z17877_PEA_1_node_17 (SEQ ID NO: 1182)

According to preferred embodiments of the present invention, there is provided an isolated polypeptide comprising an amino acid sequence in the table below

Protein Name

Z17877_PEA_1_P1 (SEQ ID NO: 1183)
Z17877_PEA_1_P2 (SEQ ID NO: 1184)
Z17877_PEA_1_P3 (SEQ ID NO: 1185)
Z17877_PEA_1_P6 (SEQ ID NO: 1186)

According to preferred embodiments of the present invention, there is provided an isolated polynucleotide comprising a nucleic acid sequence in the table below and/or:

Transcript Name

HSHCGI_PEA_3_T0 (SEQ ID NO: 1187)
HSHCGI_PEA_3_T1 (SEQ ID NO: 1188)
HSHCGI_PEA_3_T2 (SEQ ID NO: 1189)
HSHCGI_PEA_3_T3 (SEQ ID NO: 1190)
HSHCGI_PEA_3_T4 (SEQ ID NO: 1191)
HSHCGI_PEA_3_T5 (SEQ ID NO: 1192)
HSHCGI_PEA_3_T6 (SEQ ID NO: 1193)
HSHCGI_PEA_3_T7 (SEQ ID NO: 1194)
HSHCGI_PEA_3_T8 (SEQ ID NO: 1195)
HSHCGI_PEA_3_T9 (SEQ ID NO: 1196)
HSHCGI_PEA_3_T10 (SEQ ID NO: 1197)
HSHCGI_PEA_3_T11 (SEQ ID NO: 1198)
HSHCGI_PEA_3_T12 (SEQ ID NO: 1199)
HSHCGI_PEA_3_T13 (SEQ ID NO: 1200)
HSHCGI_PEA_3_T14 (SEQ ID NO: 1201)
HSHCGI_PEA_3_T15 (SEQ ID NO: 1202)
HSHCGI_PEA_3_T17 (SEQ ID NO: 1203)
HSHCGI_PEA_3_T18 (SEQ ID NO: 1204)
HSHCGI_PEA_3_T19 (SEQ ID NO: 1205)
HSHCGI_PEA_3_T20 (SEQ ID NO: 1206)
HSHCGI_PEA_3_T21 (SEQ ID NO: 1207)
HSHCGI_PEA_3_T22 (SEQ ID NO: 1208)
HSHCGI_PEA_3_T23 (SEQ ID NO: 1209)
HSHCGI_PEA_3_T24 (SEQ ID NO: 1210)

a nucleic acid sequence comprising a sequence in the table below:

Segment Name

HSHCGI_PEA_3_node_0 (SEQ ID NO: 1211)
HSHCGI_PEA_3_node_2 (SEQ ID NO: 1212)
HSHCGI_PEA_3_node_7 (SEQ ID NO: 1213)
HSHCGI_PEA_3_node_8 (SEQ ID NO: 1214)
HSHCGI_PEA_3_node_14 (SEQ ID NO: 1215)
HSHCGI_PEA_3_node_16 (SEQ ID NO: 1216)
HSHCGI_PEA_3_node_18 (SEQ ID NO: 1217)
HSHCGI_PEA_3_node_20 (SEQ ID NO: 1218)
HSHCGI_PEA_3_node_26 (SEQ ID NO: 1219)
HSHCGI_PEA_3_node_28 (SEQ ID NO: 1220)
HSHCGI_PEA_3_node_30 (SEQ ID NO: 1221)
HSHCGI_PEA_3_node_32 (SEQ ID NO: 1222)
HSHCGI_PEA_3_node_33 (SEQ ID NO: 1223)
HSHCGI_PEA_3_node_34 (SEQ ID NO: 1224)
HSHCGI_PEA_3_node_36 (SEQ ID NO: 1225)
HSHCGI_PEA_3_node_1 (SEQ ID NO: 1226)
HSHCGI_PEA_3_node_4 (SEQ ID NO: 1227)
HSHCGI_PEA_3_node_6 (SEQ ID NO: 1228)
HSHCGI_PEA_3_node_9 (SEQ ID NO: 1229)
HSHCGI_PEA_3_node_11 (SEQ ID NO: 1230)
HSHCGI_PEA_3_node_13 (SEQ ID NO: 1231)
HSHCGI_PEA_3_node_19 (SEQ ID NO: 1232)
HSHCGI_PEA_3_node_21 (SEQ ID NO: 1233)
HSHCGI_PEA_3_node_22 (SEQ ID NO: 1234)
HSHCGI_PEA_3_node_23 (SEQ ID NO: 1235)
HSHCGI_PEA_3_node_24 (SEQ ID NO: 1236)
HSHCGI_PEA_3_node_27 (SEQ ID NO: 1237)
HSHCGI_PEA_3_node_31 (SEQ ID NO: 1238)
HSHCGI_PEA_3_node_35 (SEQ ID NO: 1239)

According to preferred embodiments of the present invention, there is provided an isolated polypeptide comprising an amino acid sequence in the table below:

Protein Name

HSHCGI_PEA_3_P17 (SEQ ID NO: 1243)
HSHCGI_PEA_3_P18 (SEQ ID NO: 1244)
HSHCGI_PEA_3_P19 (SEQ ID NO: 1245)
HSHCGI_PEA_3_P1 (SEQ ID NO: 1246)
HSHCGI_PEA_3_P4 (SEQ ID NO: 1247)
HSHCGI_PEA_3_P6 (SEQ ID NO: 1248)
HSHCGI_PEA_3_P7 (SEQ ID NO: 1249)
HSHCGI_PEA_3_P8 (SEQ ID NO: 1250)
HSHCGI_PEA_3_P9 (SEQ ID NO: 1251)
HSHCGI_PEA_3_P12 (SEQ ID NO: 1252)
HSHCGI_PEA_3_P13 (SEQ ID NO: 1253)
HSHCGI_PEA_3_P14 (SEQ ID NO: 1254)
HSHCGI_PEA_3_P15 (SEQ ID NO: 1255)
HSHCGI_PEA_3_P16 (SEQ ID NO: 1256)
HSHCGI_PEA_3_P20 (SEQ ID NO: 1257)
HSHCGI_PEA_3_P21 (SEQ ID NO: 1258)
HSHCGI_PEA_3_P22 (SEQ ID NO: 1259)

According to preferred embodiments of the present invention, there is provided an isolated chimeric polypeptide encoding for HSHCGI_PEA_{—}3_P17 (SEQ ID NO:1243), comprising a first amino acid sequence being at least 90% homologous to MASGQFVNKLQEEVICPICLDILQKPVTIDCGHNFCPQCITQIGETSCGFFKCPLCKTSVRRDAIRFNSLLRNL VEKIQALQASEVQSKRKEATCPRHQEMFHYFCEDDGKFLCFVCRESKDHKSHNVSLIEEAAQNYQGQIQEQ IQVLQQKEKETVQVKAQGVHRVDVFTDQVEHEKQRILTEFELLHQVLEEEKNFLLSRIYWLGHEGTEAGKH YV corresponding to amino acids 1-218 of TM31_HUMAN (SEQ ID NO:1242), which also corresponds to amino acids 1-218 of HSHCGI_PEA_{—}3_P17 (SEQ ID NO:1243), and a second amino acid sequence being at least 70%, optionally at least 80%, preferably at least 85%, more preferably at least 90% and most preferably at least 95% homologous to a polypeptide having the sequence EIPLMPTVERSQEARCYP (SEQ ID NO:1442) corresponding to amino acids 219-236 of HSHCGI_PEA_{—}3_P17 (SEQ ID NO:1243), wherein said first amino acid sequence and second amino acid sequence are contiguous and in a sequential order.

According to preferred embodiments of the present invention, there is provided an isolated polypeptide encoding for a tail of HSHCGI_PEA_{—}3_P17 (SEQ ID NO:1243), comprising a polypeptide being at least 70%, optionally at least about 80%, preferably at least about 85%, more preferably at least about 90% and most preferably at least about 95% homologous to the sequence EIPLMPTVERSQEARCYP (SEQ ID NO:1442) in HSHCGI_PEA_{—}3_P17 (SEQ ID NO:1243).

According to preferred embodiments of the present invention, there is provided an isolated chimeric polypeptide encoding for HSHCGI_PEA_{—}3_P19 (SEQ ID NO:1245), comprising a first amino acid sequence being at least 90% homologous to MASGQFVNKLQEEVICPICLDILQKPVTIDCGHNFCLKCITQIGETSCGFFKCPLCKTSVRRDAIRFNSLLRNL VEKIQALQASEVQSKRKEATCPRHQEMFHYFCEDDGKFLCFVCRESKDHKSHNVSLIEEAAQNYQGQIQEQ IQVLQQKEKETVQVKAQGVHRVDVFTDQVEHEKQRILTEFELLHQVLEEEKNFLLSRIYWLGHEGTEAGKH YVASTEPQLNDLKKLVDSLKTKQNMPPRQLLE corresponding to amino acids 1-248 of TM31_HUMAN (SEQ ID NO:1242)_V2 (SEQ ID NO:1241), which also corresponds to amino acids 1-248 of HSHCGI_PEA_{—}3_P19 (SEQ ID NO:1245), and a second amino acid sequence being at least 70%, optionally at least 80%, preferably at least 85%, more preferably at least 90% and most preferably at least 95% homologous to a polypeptide having the sequence NWRKNSVKQNQDTTPSQGA (SEQ ID NO:1443) corresponding to amino acids 249-267 of HSHCGI_PEA_{—}3_P19 (SEQ ID NO:1245), wherein said first amino acid sequence and second amino acid sequence are contiguous and in a sequential order.

According to preferred embodiments of the present invention, there is provided an isolated polypeptide encoding for a tail of HSHCGI_PEA_{—}3_P19 (SEQ ID NO:1245), comprising a polypeptide being at least 70%, optionally at least about 80%, preferably at least about 85%, more preferably at least about 90% and most preferably at least about 95% homologous to the sequence NWRKNSVKQNQDTTPSQGA (SEQ ID NO:1443) in HSHCGI_PEA_{—}3_P19 (SEQ ID NO:1245).

According to preferred embodiments of the present invention, there is provided an isolated chimeric polypeptide encoding for HSHCGI_PEA_{—}3_P4 (SEQ ID NO:1247), comprising a first amino acid sequence being at least 90% homologous to MASGQFVNKLQEEVICPICLDILQKPVTIDCGHNFCLKCITQIGETSCGFFKCPLCKTSVRKNAIRFNSLLRNL VEKIQALQASEVQSKRKEATCPRHQEMFHYFCEDDGKFLCFVCRESKDHKSHNVSLIEEAAQNYQGQIQEQ IQVLQQKEKETVQVKAQGVHRVDVFTDQVEHEKQRILTEFELLHQVLEEEKNFLLSRIYWLGHEGTEAGKH YVASTEPQLNDLKKLVDSLKTKQNMPPRQLLEDIKVVLCR corresponding to amino acids 1-256 of TM31_HUMAN_V1 (SEQ ID NO:1240), which also corresponds to amino acids 1-256 of HSHCGI_PEA_{—}3_P4 (SEQ ID NO:1247), and a second amino acid sequence being at least 70%, optionally at least 80%, preferably at least 85%, more preferably at least 90% and most preferably at least 95% homologous to a polypeptide having the sequence YDGPPQMYFAY (SEQ ID NO:1444) corresponding to amino acids 257-267 of HSHCGI_PEA_{—}3_P4 (SEQ ID NO:1247), wherein said first amino acid sequence and second amino acid sequence are contiguous and in a sequential order.

According to preferred embodiments of the present invention, there is provided an isolated polypeptide encoding for a tail of HSHCGI_PEA_{—}3_P4 (SEQ ID NO:1247), comprising a polypeptide being at least 70%, optionally at least about 80%, preferably at least about 85%, more preferably at least about 90% and most preferably at least about 95% homologous to the sequence YDGPPQMYFAY (SEQ ID NO:1444) in HSHCGI_PEA_{—}3_P4 (SEQ ID NO:1247).

According to preferred embodiments of the present invention, there is provided an isolated chimeric polypeptide encoding for HSHCGI_PEA_{—}3_P6 (SEQ ID NO:1248), comprising a first amino acid sequence being at least 90% homologous to MASGQFVNKLQEEVICPICLDILQKPVTIDCGHNFCLKCITQIGETSCGFFKCPLCKTSVRKNAIRFNSLLRNL VEKIQALQASEVQSKRKEATCPRHQEMFHYFCEDDGKFLCFVCRESKDHKSHNVSLIEEAAQNYQGQIQEQ IQVLQQKEKETVQVKAQGVHRVDVFTDQVEHEKQRILTEFELLHQVLEEEKNFLLSRIYWLGHEGTEAGKH YVASTEPQLNDLKKLVDSLKTKQNMPPRQLLEDIKVVLCR corresponding to amino acids 1-256 of TM31_HUMAN_V1 (SEQ ID NO:1240), which also corresponds to amino acids 1-256 of HSHCGI_PEA_{—}3_P6 (SEQ ID NO:1248), and a second amino acid sequence being at least 70%, optionally at least 80%, preferably at least 85%, more preferably at least 90% and most preferably at least 95% homologous to a polypeptide having the sequence PTPG (SEQ ID NO:1445) corresponding to amino acids 257-260 of HSHCGI_PEA_{—}3_P6 (SEQ ID NO:1248), wherein said first amino acid sequence and second amino acid sequence are contiguous and in a sequential order.

According to preferred embodiments of the present invention, there is provided an isolated polypeptide encoding for a tail of HSHCGI_PEA_{—}3_P6 (SEQ ID NO:1248), comprising a polypeptide being at least 70%, optionally at least about 80%, preferably at least about 85%, more preferably at least about 90% and most preferably at least about 95% homologous to the sequence PTPG (SEQ ID NO:1445) in HSHCGI_PEA_{—}3_P6 (SEQ ID NO:1248).

According to preferred embodiments of the present invention, there is provided an isolated chimeric polypeptide encoding for HSHCGI_PEA_{—}3_P7 (SEQ ID NO:1249), comprising a first amino acid sequence being at least 90% homologous to MASGQFVNKLQEEVICPICLDILQKPVTIDCGHNFCLKCITQIGETSCGFFKCPLCKTSVRKNAIRFNSLLRNL VEKIQALQASEVQSKRKEATCPRHQEMFHYFCEDDGKFLCFVCRESKDHKSHNVSLIEEAAQNYQGQIQEQ IQVLQQKEKETVQVKAQGVHRVDVFTDQVEHEKQRILTEFELLHQVLEEEKNFLLSRIYWLGHEGTEAGKH YVASTEPQLNDLKKLVDSLKTKQNMPPRQLLEDIKVVLCRS corresponding to amino acids 1-257 of TM31_HUMAN_V1 (SEQ ID NO:1240), which also corresponds to amino acids 1-257 of HSHCGI_PEA_{—}3_P7 (SEQ ID NO:1249), and a second amino acid sequence being at least 70%, optionally at least 80%, preferably at least 85%, more preferably at least 90% and most preferably at least 95% homologous to a polypeptide having the sequence SFSHTSSPDLTNQLNHIFLEVKSFSFSTQPLFLWNWRKNSVKQNQDTTPSQGA (SEQ ID NO:1446) corresponding to amino acids 258-310 of HSHCGI_PEA_{—}3_P7 (SEQ ID NO:1249), wherein said first amino acid sequence and second amino acid sequence are contiguous and in a sequential order.

According to preferred embodiments of the present invention, there is provided an isolated polypeptide encoding for a tail of HSHCGI_PEA_{—}3_P7 (SEQ ID NO:1249), comprising a polypeptide being at least 70%, optionally at least about 80%, preferably at least about 85%, more preferably at least about 90% and most preferably at least about 95% homologous to the sequence SFSHTSSPDLTNQLNHIFLEVKSFSFSTQPLFLWNWRKNSVKQNQDTTPSQGA (SEQ ID NO:1446) in HSHCGI_PEA_{—}3_P7 (SEQ ID NO:1249).

According to preferred embodiments of the present invention, there is provided an isolated chimeric polypeptide encoding for HSHCGI_PEA_{—}3_P8 (SEQ ID NO:1250), comprising a first amino acid sequence being at least 90% homologous to MASGQFVNKLQEEVICPICLDILQKPVTIDCGHNFCLKCITQIGETSCGFFKCPLCKTSVRKNAIRFNSLLRNL VEKIQALQASEVQSKRKEATCPRHQEMFHYFCEDDGKFLCFVCRESKDHKSHNVSLIEEAAQNYQGQIQEQ IQVLQQKEKETVQVKAQGVHRVDVFTDQVEHEKQRILTEFELLHQVLEEEKNFLLSRIYWLGHEGTEAGKH YVASTEPQLNDLKKLVDSLKTKQNMPPRQLLEDIKVVLCRSEEFQFLNPTPVPLELEKKLSEAKSRHDSITGS LKKFKDQLQADRKKDENRFFKSMNKNDMKSWGLLQKNNHKMNKTSEPGSSSAG corresponding to amino acids 1-342 of TM31_HUMAN_V1 (SEQ ID NO:1240), which also corresponds to amino acids 343-349 of HSHCGI_PEA_{—}3_P8 (SEQ ID NO:1250), and a second amino acid sequence being at least 70%, optionally at least 80%, preferably at least 85%, more preferably at least 90% and most preferably at least 95% homologous to a polypeptide having the sequence KSPVSEY (SEQ ID NO:1447) corresponding to amino acids 343-349 of HSHCGI_PEA_{—}3_P8 (SEQ ID NO:1250), wherein said first amino acid sequence and second amino acid sequence are contiguous and in a sequential order.

According to preferred embodiments of the present invention, there is provided an isolated polypeptide encoding for a tail of HSHCGI_PEA_{—}3_P8 (SEQ ID NO:1250), comprising a polypeptide being at least 70%, optionally at least about 80%, preferably at least about 85%, more preferably at least about 90% and most preferably at least about 95% homologous to the sequence KSPVSEY (SEQ ID NO:1447) in HSHCGI_PEA_{—}3_P8 (SEQ ID NO:1250).

According to preferred embodiments of the present invention, there is provided an isolated chimeric polypeptide encoding for HSHCGI_PEA_{—}3_P9 (SEQ ID NO:1251), comprising a first amino acid sequence being at least 90% homologous to MASGQFVNKLQEEVICPICLDILQKPVTIDCGHNFCLKCITQIGETSCGFFKCPLCKTSVRKNAIRFNSLLRNL VEKIQALQASEVQSKRKEATCPRHQEMFHYFCEDDGKFLCFVCRESKDHKSHNVSLIEEAAQNYQGQIQEQ IQVLQQKEKETVQVKAQGVHRVDVFTDQVEHEKQRILTEFELLHQVLEEEKNFLLSRIYWLGHEGTEAGKH YVASTEPQLNDLKKLVDSLKTKQNMPPRQLLEDIKVVLCR corresponding to amino acids 1-256 of TM31_HUMAN_V1 (SEQ ID NO:1240), which also corresponds to amino acids 1-256 of HSHCGI_PEA_{—}3_P9 (SEQ ID NO:1251), and a second amino acid sequence being at least 70%, optionally at least 80%, preferably at least 85%, more preferably at least 90% and most preferably at least 95% homologous to a polypeptide having the sequence TGEKTQ (SEQ ID NO:1448) corresponding to amino acids 257-262 of HSHCGI_PEA_{—}3_P9 (SEQ ID NO:1251), wherein said first amino acid sequence and second amino acid sequence are contiguous and in a sequential order.

According to preferred embodiments of the present invention, there is provided an isolated polypeptide encoding for a tail of HSHCGI_PEA_{—}3_P9 (SEQ ID NO:1251), comprising a polypeptide being at least 70%, optionally at least about 80%, preferably at least about 85%, more preferably at least about 90% and most preferably at least about 95% homologous to the sequence TGEKTQ (SEQ ID NO:1448) in HSHCGI_PEA_{—}3_P9 (SEQ ID NO:1251).

According to preferred embodiments of the present invention, there is provided an isolated chimeric polypeptide encoding for HSHCGI_PEA_{—}3_P12 (SEQ ID NO:1252), comprising a first amino acid sequence being at least 90% homologous to MNKNDMKSWGLLQKNNHKMNKTSEPGSSSAGGRTTSGPPNHHSSAPSHSLFRASSAGKVTFPVCLLASYD EISGQGASSQDTKTFDVALSEELHAALSEWLTAIRAWFCEVPSS corresponding to amino acids 312-425 of TM31_HUMAN (SEQ ID NO:1242), which also corresponds to amino acids 1-114 of HSHCGI_PEA_{—}3_P12 (SEQ ID NO:1252).

According to preferred embodiments of the present invention, there is provided an isolated chimeric polypeptide encoding for HSHCGI_PEA_{—}3_P14 (SEQ ID NO:1254), comprising a first amino acid sequence being at least 90% homologous to MASGQFVNKLQEEVICPICLDILQKPVTIDCGHNFCLKCITQIGETSCGFFKCPLCKTSVRKNAIRFNSLLRNL VEKIQALQASEVQSKRKEATCPRHQEMFHYFCEDDGKFLCFVCRESKDHKSHNVSLIEEAAQNYQGQIQEQ IQVLQQKEKETVQVKAQGVHRVDVFTDQVEHEKQRILTEFELLHQVLEEEKNFLLSRIYWLGHEGTEAGKH YVASTEPQLNDLKKLVDSLKTKQNMPPRQLLEDIKVVLCRSEEFQFLNPTPVPLELEKKLSEAKSRHDSITGS LKKFKDQLQADRKKDENRFFKSMNKNDMKS corresponding to amino acids 1-319 of TM31_HUMAN_V1 (SEQ ID NO:1240), which also corresponds to amino acids 1-319 of HSHCGI_PEA_{—}3_P14 (SEQ ID NO:1254), and a second amino acid sequence being at least 70%, optionally at least 80%, preferably at least 85%, more preferably at least 90% and most preferably at least 95% homologous to a polypeptide having the sequence CK corresponding to amino acids 320-321 of HSHCGI_PEA_{—}3_P14 (SEQ ID NO:1254), wherein said first amino acid sequence and second amino acid sequence are contiguous and in a sequential order.

According to preferred embodiments of the present invention, there is provided an isolated chimeric polypeptide encoding for HSHCGI_PEA_{—}3_P16 (SEQ ID NO:1256), comprising a first amino acid sequence being at least 90% homologous to MASGQFVNKLQEEVICPICLDILQKPVTIDCGHNFCLKCITQIGETSCGFFKCPLCKTSVRKNAIRFNSLLRNL VEKIQALQASEVQSKRKEATCPRHQEMFHYFCEDDGKFLCFVCRESKDHKSHNVSLIEEAAQNYQGQIQEQ IQVLQQKEKETVQVKAQGVHRVDVFT corresponding to amino acids 1-171 of TM31_HUMAN_V1 (SEQ ID NO:1240), which also corresponds to amino acids 1-171 of HSHCGI_PEA_{—}3_P16 (SEQ ID NO:1256), and a second amino acid sequence being at least 70%, optionally at least 80%, preferably at least 85%, more preferably at least 90% and most preferably at least 95% homologous to a polypeptide having the sequence VRKTPSHDLWKQKHLCQSSWNPLLH (SEQ ID NO:1449) corresponding to amino acids 172-196 of HSHCGI_PEA_{—}3_P16 (SEQ ID NO:1256), wherein said first amino acid sequence and second amino acid sequence are contiguous and in a sequential order.

According to preferred embodiments of the present invention, there is provided an isolated polypeptide encoding for a tail of HSHCGI_PEA_{—}3_P16 (SEQ ID NO:1256), comprising a polypeptide being at least 70%, optionally at least about 80%, preferably at least about 85%, more preferably at least about 90% and most preferably at least about 95% homologous to the sequence VRKTPSHDLWKQKHLCQSSWNPLLH (SEQ ID NO:1449) in HSHCGI_PEA_{—}3_P16 (SEQ ID NO:1256).

According to preferred embodiments of the present invention, there is provided an isolated chimeric polypeptide encoding for HSHCGI_PEA_{—}3_P21 (SEQ ID NO:1258), comprising a first amino acid sequence being at least 70%, optionally at least 80%, preferably at least 85%, more preferably at least 90% and most preferably at least 95% homologous to a polypeptide having the sequence MHHSDWGNIMWIFQMSPLQNFRKEERNQ (SEQ ID NO:1450) corresponding to amino acids 1-28 of HSHCGI_PEA_{—}3_P21 (SEQ ID NO:1258), and a second amino acid sequence being at least 90% homologous to FLCFVCRESKDHKSHNVSLIEEAAQNYQGQIQEQIQVLQQKEKETVQVKAQGVHRVDVFTDQVEHEKQRIL TEFELLHQVLEEEKNFLLSRIYWLGHEGTEAGKHYVASTEPQLNDLKKLVDSLKTKQNMPPRQLLEDIKvv LCRSEEFQFLNPTPVPLELEKKLSEAKSRHDSITGSLKKFKDQLQADRKKDENRFFKSMNKNDMKSWGLLQ KNNHKMNKTSEPGSSSAGGRTTSGPPNHHSSAPSHSLFRASSAGKVTFPVCLLASYDEISGQGASSQDTKTF DVALSEELHAALSEWLTAIRAWFCEVPSS corresponding to amino acids 112-425 of TM31_HUMAN (SEQ ID NO:1242), which also corresponds to amino acids 29-342 of HSHCGI_PEA_{—}3_P21 (SEQ ID NO:1258), wherein said first amino acid sequence and second amino acid sequence are contiguous and in a sequential order.

According to preferred embodiments of the present invention, there is provided an isolated polypeptide encoding for a head of HSHCGI_PEA_{—}3_P21 (SEQ ID NO:1258), comprising a polypeptide being at least 70%, optionally at least about 80%, preferably at least about 85%, more preferably at least about 90% and most preferably at least about 95% homologous to the sequence MHHSDWGNIMWIFQMSPLQNFRKEERNQ (SEQ ID NO:1450) of HSHCGI_PEA_{—}3_P21 (SEQ ID NO:1258).

According to preferred embodiments of the present invention, there is provided an isolated chimeric polypeptide encoding for HSHCGI_PEA_{—}3_P22 (SEQ ID NO:1259), comprising a first amino acid sequence being at least 90% homologous to MPPRQLLEDIKVVLCRSEEFQFLNPTPVPLELEKKLSEAKSRHDSITGSLKKFKDQLQADRKKDENRFFKSM NKNDMKSWGLLQKNNHKMNKTSEPGSSSAGGRTTSGPPNHHSSAPSHSLFRASSAGKVTFPVCLLASYDEI SGQGASSQDTKTFDVALSEELHAALSEWLTAIRAWFCEVPSS corresponding to amino acids 241-425 of TM31_HUMAN (SEQ ID NO:1242), which also corresponds to amino acids 1-185 of HSHCGI_PEA_{—}3_P22 (SEQ ID NO:1259).

According to preferred embodiments of the present invention, there is provided an isolated chimeric polypeptide encoding for T51958_PEA_{—}1_P5 (SEQ ID NO:1151), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQPQ TQVTLRCHIDGHPRPTYQWFRDGTPLSDGQSNHTVSSKERNLTLRPAGPEHSGLYSCCAHSAFGQACSSQNF TLSIADESFARVVLAPQDVVVARYEEAMFHCQFSAQPPPSLQWLFEDETPITNRSRPPHLRRATVFANGSLLL TQVRPRNAGIYRCIGQGQRGPPIILEATLHLAEIEDMPLFEPRVFTAGSEERVTCLPPKGLPEPSVWWEHAGV RLPTHGRVYQKGHELVLANIAESDAGVYTCHAANLAGQRRQDVNITVATVPSWLKKPQDSQLEEGKPGYL DCLTQATPKPTVVWYRNQMLISEDSRFEVFKNGTLRINSVEVYDGTWYRCMSSTPAGSIEAQARVQVLEKL KFTPPPQPQQCMEFDKEATVPCSATGREKPTIKWERADGSSLPEWVTDNAGTLHFARVTRDDAGNYTCIAS NGPQGQIRAHVQLTVAVFITFKVEPERTTVYQGHTALLQCEAQGDPKPLIQWKGKDRILDPTKLGPRMHIFQ NGSLVIHDVAPEDSGRYTCIAGNSCNIKHTEAPLYVV corresponding to amino acids 1-682 of PTK7_HUMAN_V4 (SEQ ID NO:1143), which also corresponds to amino acids 1-682 of T51958_PEA_{—}1_P5 (SEQ ID NO:1151), and a second amino acid sequence being at least 70%, optionally at least 80%, preferably at least 85%, more preferably at least 90% and most preferably at least 95% homologous to a polypeptide having the sequence GMGWGGLCCTGSGGPRRLSPCTQPLCTEHGTEAIFVAAVGIRPSHHAAAQS (SEQ ID NO:1451) corresponding to amino acids 683-733 of T51958_PEA_{—}1_P5 (SEQ ID NO:1151), wherein said first amino acid sequence and second amino acid sequence are contiguous and in a sequential order.

According to preferred embodiments of the present invention, there is provided an isolated polypeptide encoding for a tail of T51958_PEA_{—}1_P5 (SEQ ID NO:1151), comprising a polypeptide being at least 70%, optionally at least about 80%, preferably at least about 85%, more preferably at least about 90% and most preferably at least about 95% homologous to the sequence GMGWGGLCCTGSGGPRRLSPCTQPLCTEHGTEAIFVAAVGIRPSHHAAAQS (SEQ ID NO:1451) in T51958_PEA_{—}1_P5 (SEQ ID NO:1151).

According to preferred embodiments of the present invention, there is provided an isolated chimeric polypeptide encoding for T51958_PEA_{—}1_P6 (SEQ ID NO:1152), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQPQ TQVTLRCHIDGHPRPTYQWFRDGTPLSDGQSNHTVSSKERNLTLRPAGPEHSGLYSCCAHSAFGQACSSQNF TLSIADESFARVVLAPQDVVVARYEEAMFHCQFSAQPPPSLQWLFEDETPITNRSRPPHLRRATVFANGSLLL TQVRPRNAGIYRCIGQGQRGPPIILEATLHLAEIEDMPLFEPRVFTAGSEERVTCLPPKGLPEPSVWWEHAGV RLPTHGRVYQKGHELVLANIAESDAGVYTCHAANLAGQRRQDVNITVATVPSWLKKPQDSQLEEGKPGYL DCLTQATPKPTVVWYRNQMLISEDSRFEVFKNGTLRINSVEVYDGTWYRCMSSTPAGSIEAQARVQVLEKL KFTPPPQPQQCMEFDKEATVPCSATGREKPTIKWERADGSSLPEWVTDNAGTLHFARVTRDDAGNYTCIAS NGPQGQIRAHVQLTVAVFITFKVEPERTTVYQGHTALLQCEAQGDPKPLIQWKGKDRILDPTKLGPRM corresponding to amino acids 1-641 of PTK7_HUMAN_V4 (SEQ ID NO:1143), which also corresponds to amino acids 1-641 of T51958_PEA_{—}1_P6 (SEQ ID NO:1152), and a second amino acid sequence being at least 70%, optionally at least 80%, preferably at least 85%, more preferably at least 90% and most preferably at least 95% homologous to a polypeptide having the sequence APW corresponding to amino acids 642-644 of T51958_PEA_{—}1_P6 (SEQ ID NO:1152), wherein said first amino acid sequence and second amino acid sequence are contiguous and in a sequential order.

According to preferred embodiments of the present invention, there is provided an isolated chimeric polypeptide encoding for T51958_PEA_{—}1_P28 (SEQ ID NO:1153), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQPQ TQVTLRCHIDGHPRPTYQWFRDGTPLSDGQSNHTVSSKERNLTLRPAGPEHSGLYSCCAHSAFGQACSSQNF TLSIADESFARVVLAPQDVVVARYEEAMFHCQFSAQPPPSLQWLFEDETPITNRSRPPHLRRATVFANGSLLL TQVRPRNAGIYRCIGQGQRGPPIILEATLHLAEIEDMPLFEPRVFTAGSEERVTCLPPKGLPEPSVWWEHAGV RLPTHGRVYQKGHELVLANIAESDAGVYTCHAANLAGQRRQDVNITVA corresponding to amino acids 1-409 of PTK7_HUMAN_V11 (SEQ ID NO:1144), which also corresponds to amino acids 1-409 of T51958_PEA_{—}1_P28 (SEQ ID NO:1153), and a second amino acid sequence being at least 70%, optionally at least 80%, preferably at least 85%, more preferably at least 90% and most preferably at least 95% homologous to a polypeptide having the sequence SEHLCPEGQGEVEGNTGLGVMDRGFPGTHLRSSQFWALQAWESVHYWESV (SEQ ID NO:1452) corresponding to amino acids 410-459 of T51958_PEA_{—}1_P28 (SEQ ID NO:1153), wherein said first amino acid sequence and second amino acid sequence are contiguous and in a sequential order.

According to preferred embodiments of the present invention, there is provided an isolated polypeptide encoding for a tail of T51958_PEA_{—}1_P28 (SEQ ID NO:1153), comprising a polypeptide being at least 70%, optionally at least about 80%, preferably at least about 85%, more preferably at least about 90% and most preferably at least about 95% homologous to the sequence SEHLCPEGQGEVEGNTGLGVMDRGFPGTHLRSSQFWALQAWESVHYWESV (SEQ ID NO:1452) in T51958_PEA_{—}1_P28 (SEQ ID NO:1153).

According to preferred embodiments of the present invention, there is provided an isolated chimeric polypeptide encoding for T51958_PEA_{—}1_P28 (SEQ ID NO:1153), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQPQ TQVTLRCHIDGHPRPTYQWFRDGTPLSDGQSNHTVSSKERNLTLRPAGPEHSGLYSCCAHSAFGQACSSQNF TLSIADESFARVVLAPQDVVVARYEEAMFHCQFSAQPPPSLQWLFEDETPITNRSRPPHLRRATVFANGSLLL TQVRPRNAGIYRCIGQGQRGPPIILEATLHLAEIEDMPLFEPRVFTAGSEERVTCLPPKGLPEPSVWWEHAGV RLPTHGRVYQKGHELVLANIAESDAGVYTCHAANLAGQRRQDVNITVA corresponding to amino acids 1-409 of Q8NFA5 (SEQ ID NO:1147), which also corresponds to amino acids 1-409 of T51958_PEA_{—}1_P28 (SEQ ID NO:1153), and a second amino acid sequence being at least 70%, optionally at least 80%, preferably at least 85%, more preferably at least 90% and most preferably at least 95% homologous to a polypeptide having the sequence SEHLCPEGQGEVEGNTGLGVMDRGFPGTHLRSSQFWALQAWESVHYWESV (SEQ ID NO:1452) corresponding to amino acids 410-459 of T51958_PEA_{—}1_P28 (SEQ ID NO:1153), wherein said first amino acid sequence and second amino acid sequence are contiguous and in a sequential order.

According to preferred embodiments of the present invention, there is provided an isolated polypeptide encoding for a tail of T51958_PEA_{—}1_P28 (SEQ ID NO:1153), comprising a polypeptide being at least 70%, optionally at least about 80%, preferably at least about 85%, more preferably at least about 90% and most preferably at least about 95% homologous to the sequence SEHLCPEGQGEVEGNTGLGVMDRGFPGTHLRSSQFWALQAWESVHYWESV (SEQ ID NO:1452) in T51958_PEA_{—}1_P28 (SEQ ID NO:1153).

According to preferred embodiments of the present invention, there is provided an isolated chimeric polypeptide encoding for T51958_PEA_{—}1_P28 (SEQ ID NO:1153), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQPQ TQVTLRCHIDGHPRPTYQWFRDGTPLSDGQSNHTVSSKERNLTLRPAGPEHSGLYSCCAHSAFGQACSSQNF TLSIADESFARVVLAPQDVVVARYEEAMFHCQFSAQPPPSLQWLFEDETPITNRSRPPHLRRATVFANGSLLL TQVRPRNAGIYRCIGQGQRGPPIILEATLHLAEIEDMPLFEPRVFTAGSEERVTCLPPKGLPEPSVWWEHAGV RLPTHGRVYQKGHELVLANIAESDAGVYTCHAANLAGQRRQDVNITVA corresponding to amino acids 1-409 of Q8NFA6 (SEQ ID NO:1149), which also corresponds to amino acids 1-409 of T51958_PEA_{—}1_P28 (SEQ ID NO:1153), and a second amino acid sequence being at least 70%, optionally at least 80%, preferably at least 85%, more preferably at least 90% and most preferably at least 95% homologous to a polypeptide having the sequence SEHLCPEGQGEVEGNTGLGVMDRGFPGTHLRSSQFWALQAWESVHYWESV (SEQ ID NO:1452) corresponding to amino acids 410-459 of T51958_PEA_{—}1_P28 (SEQ ID NO:1153), wherein said first amino acid sequence and second amino acid sequence are contiguous and in a sequential order.

According to preferred embodiments of the present invention, there is provided an isolated polypeptide encoding for a tail of T51958_PEA_{—}1_P28 (SEQ ID NO:1153), comprising a polypeptide being at least 70%, optionally at least about 80%, preferably at least about 85%, more preferably at least about 90% and most preferably at least about 95% homologous to the sequence SEHLCPEGQGEVEGNTGLGVMDRGFPGTHLRSSQFWALQAWESVHYWESV (SEQ ID NO:1452) in T51958_PEA_{—}1_P28 (SEQ ID NO:1153).

According to preferred embodiments of the present invention, there is provided an isolated chimeric polypeptide encoding for T51958_PEA_{—}1_P28 (SEQ ID NO:1153), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQPQ TQVTLRCHIDGHPRPTYQWFRDGTPLSDGQSNHTVSSKERNLTLRPAGPEHSGLYSCCAHSAFGQACSSQNF TLSIADESFARVVLAPQDVVVARYEEAMFHCQFSAQPPPSLQWLFEDETPITNRSRPPHLRRATVFANGSLLL TQVRPRNAGIYRCIGQGQRGPPIILEATLHLAEIEDMPLFEPRVFTAGSEERVTCLPPKGLPEPSVWWEHAGV RLPTHGRVYQKGHELVLANIAESDAGVYTCHAANLAGQRRQDVNITVA corresponding to amino acids 1-409 of Q8NFA7 (SEQ ID NO:1148), which also corresponds to amino acids 1-409 of T51958_PEA_{—}1_P28 (SEQ ID NO:1153), and a second amino acid sequence being at least 70%, optionally at least 80%, preferably at least 85%, more preferably at least 90% and most preferably at least 95% homologous to a polypeptide having the sequence SEHLCPEGQGEVEGNTGLGVMDRGFPGTHLRSSQFWALQAWESVHYWESV (SEQ ID NO:1452) corresponding to amino acids 410-459 of T51958_PEA_{—}1_P28 (SEQ ID NO:1153), wherein said first amino acid sequence and second amino acid sequence are contiguous and in a sequential order.

According to preferred embodiments of the present invention, there is provided an isolated polypeptide encoding for a tail of T51958_PEA_{—}1_P28 (SEQ ID NO:1153), comprising a polypeptide being at least 70%, optionally at least about 80%, preferably at least about 85%, more preferably at least about 90% and most preferably at least about 95% homologous to the sequence SEHLCPEGQGEVEGNTGLGVMDRGFPGTHLRSSQFWALQAWESVHYWESV (SEQ ID NO:1452) in T51958_PEA_{—}1_P28 (SEQ ID NO:1153).

According to preferred embodiments of the present invention, there is provided an isolated chimeric polypeptide encoding for T51958_PEA_{—}1_P28 (SEQ ID NO:1153), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQPQ TQVTLRCHIDGHPRPTYQWFRDGTPLSDGQSNHTVSSKERNLTLRPAGPEHSGLYSCCAHSAFGQACSSQNF TLSIADESFARVVLAPQDVVVARYEEAMFHCQFSAQPPPSLQWLFEDETPITNRSRPPHLRRATVFANGSLLL TQVRPRNAGIYRCIGQGQRGPPIILEATLHLAEIEDMPLFEPRVFTAGSEERVTCLPPKGLPEPSVWWEHAGV RLPTHGRVYQKGHELVLANIAESDAGVYTCHAANLAGQRRQDVNITVA corresponding to amino acids 1-409 of Q8NFA8 (SEQ ID NO:1146), which also corresponds to amino acids 1-409 of T51958_PEA_{—}1_P28 (SEQ ID NO:1153), and a second amino acid sequence being at least 70%, optionally at least 80%, preferably at least 85%, more preferably at least 90% and most preferably at least 95% homologous to a polypeptide having the sequence SEHLCPEGQGEVEGNTGLGVMDRGFPGTHLRSSQFWALQAWESVHYWESV (SEQ ID NO:1452) corresponding to amino acids 410-459 of T51958_PEA_{—}1_P28 (SEQ ID NO:1153), wherein said first amino acid sequence and second amino acid sequence are contiguous and in a sequential order.

According to preferred embodiments of the present invention, there is provided an isolated polypeptide encoding for a tail of T51958_PEA_{—}1_P28 (SEQ ID NO:1153), comprising a polypeptide being at least 70%, optionally at least about 80%, preferably at least about 85%, more preferably at least about 90% and most preferably at least about 95% homologous to the sequence SEHLCPEGQGEVEGNTGLGVMDRGFPGTHLRSSQFWALQAWESVHYWESV (SEQ ID NO:1452) in T51958_PEA_{—}1_P28 (SEQ ID NO:1153).

According to preferred embodiments of the present invention, there is provided an isolated chimeric polypeptide encoding for T51958_PEA_{—}1_P28 (SEQ ID NO:1153), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQPQ TQVTLRCHIDGHPRPTYQWFRDGTPLSDGQSNHTVSSKERNLTLRPAGPEHSGLYSCCAHSAFGQACSSQNF TLSIADESFARVVLAPQDVVVARYEEAMFHCQFSAQPPPSLQWLFEDETPITNRSRPPHLRRATVFANGSLLL TQVRPRNAGIYRCIGQGQRGPPIILEATLHLAEIEDMPLFEPRVFTAGSEERVTCLPPKGLPEPSVWWEHAGV RLPTHGRVYQKGHELVLANIAESDAGVYTCHAANLAGQRRQDVNITVA corresponding to amino acids 1-409 of AAN04862 (SEQ ID NO:1150), which also corresponds to amino acids 1-409 of T51958_PEA_{—}1_P28 (SEQ ID NO:1153), and a second amino acid sequence being at least 70%, optionally at least 80%, preferably at least 85%, more preferably at least 90% and most preferably at least 95% homologous to a polypeptide having the sequence SEHLCPEGQGEVEGNTGLGVMDRGFPGTHLRSSQFWALQAWESVHYWESV (SEQ ID NO:1452) corresponding to amino acids 410-459 of T51958_PEA_{—}1_P28 (SEQ ID NO:1153), wherein said first amino acid sequence and second amino acid sequence are contiguous and in a sequential order.

According to preferred embodiments of the present invention, there is provided an isolated polypeptide encoding for a tail of T51958_PEA_{—}1_P28 (SEQ ID NO:1153), comprising a polypeptide being at least 70%, optionally at least about 80%, preferably at least about 85%, more preferably at least about 90% and most preferably at least about 95% homologous to the sequence SEHLCPEGQGEVEGNTGLGVMDRGFPGTHLRSSQFWALQAWESVHYWESV (SEQ ID NO:1452) in T51958_PEA_{—}1_P28 (SEQ ID NO:1153).

According to preferred embodiments of the present invention, there is provided an isolated chimeric polypeptide encoding for T51958_PEA_{—}1_P30 (SEQ ID NO:1154), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIK corresponding to amino acids 1-122 of PTK7_HUMAN (SEQ ID NO:1141)_V13 (SEQ ID NO:1145), which also corresponds to amino acids 1-122 of T51958_PEA_{—}1_P30 (SEQ ID NO:1154), and a second amino acid sequence being at least 70%, optionally at least 80%, preferably at least 85%, more preferably at least 90% and most preferably at least 95% homologous to a polypeptide having the sequence CESQGGCAQSPCQTLND (SEQ ID NO:1453) corresponding to amino acids 123-139 of T51958_PEA_{—}1_P30 (SEQ ID NO:1154), wherein said first amino acid sequence and second amino acid sequence are contiguous and in a sequential order.

According to preferred embodiments of the present invention, there is provided an isolated polypeptide encoding for a tail of T51958_PEA_{—}1_P30 (SEQ ID NO:1154), comprising a polypeptide being at least 70%, optionally at least about 80%, preferably at least about 85%, more preferably at least about 90% and most preferably at least about 95% homologous to the sequence CESQGGCAQSPCQTLND (SEQ ID NO:1453) in T51958_PEA_{—}1_P30 (SEQ ID NO:1154).

According to preferred embodiments of the present invention, there is provided an isolated chimeric polypeptide encoding for T51958_PEA_{—}1_P34 (SEQ ID NO:1155), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQPQ TQVTLRCHIDGHPR corresponding to amino acids 1-157 of PTK7_HUMAN_V3 (SEQ ID NO:1142), which also corresponds to amino acids 1-157 of T51958_PEA_{—}1_P34 (SEQ ID NO:1155).

According to preferred embodiments of the present invention, there is provided an isolated chimeric polypeptide encoding for T51958_PEA_{—}1_P35 (SEQ ID NO:1156), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQPQ TQVTLRCHIDGHPRPTYQWFRDGTPLSDGQSNHTVSSKERNLTLRPAGPEHSGLYSCCAHSAFGQACSSQNF TLSIA corresponding to amino acids 1-220 of PTK7_HUMAN_V11 (SEQ ID NO:1144), which also corresponds to amino acids 1-220 of T51958_PEA_{—}1_P35 (SEQ ID NO:1156), and a second amino acid sequence being at least 70%, optionally at least 80%, preferably at least 85%, more preferably at least 90% and most preferably at least 95% homologous to a polypeptide having the sequence GEPGVGAEGMR (SEQ ID NO:1454) corresponding to amino acids 221-231 of T51958_PEA_{—}1_P35 (SEQ ID NO:1156), wherein said first amino acid sequence and second amino acid sequence are contiguous and in a sequential order.

According to preferred embodiments of the present invention, there is provided an isolated polypeptide encoding for a tail of T51958_PEA_{—}1_P35 (SEQ ID NO:1156), comprising a polypeptide being at least 70%, optionally at least about 80%, preferably at least about 85%, more preferably at least about 90% and most preferably at least about 95% homologous to the sequence GEPGVGAEGMR (SEQ ID NO:1454) in T51958_PEA_{—}1_P35 (SEQ ID NO:1156).

According to preferred embodiments of the present invention, there is provided an isolated chimeric polypeptide encoding for T23657_P2 (SEQ ID NO:1064), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARGT HEVRYVSAGQSVACGWWAFAPPCLQVLNTPKGILFFLCAAAFLQGMTVNGFINTVITSLERRYDLHSYQSG LIASSYDIAACLCLTFVSYFGGSGHKPRWLGWGVLLMGTGSLVFALPHFTAGRYEVELDAGVRTCPANPGA VCADSTSGLSRYQLVFMLGQFLHGVGATPLYTLGVTYLDENVKSSCSPVYIAIFYTAAILGPAAGYLIGGAL LNIYTEMGRRTELTTESPLWVGAWWVGFLGSGAAAFFTAVPILGYPRQLPGSQRYAVMRAAEMHQLKDSS RGEASNPDFGKTIRDLPLSIWLLLKNPTFILLCLAGATEATLITGMSTFSPKFLESQFSLSASEAATLFGYLVVP AGGGGTFLGGFFVNKLRLRGSAVIKFCLFCTVVSLLGILVFSLHCPSVPMAGVTASYGGSLLPEGHLNLTAP CNAACSCQPEHYSPVCGSDGLMYFSLCHAGCPAATETNVDGQKVYRDCSCIPQNLSSGFGHATAGKCTSTC QRKPLLLVFIFVVIFFTFLSSIPALTATLRCVRDPQRSFALGIQWIVVRILGGIPGPIAFGWVIDKACLLWQDQC GQQGSCLVYQNSAMSRYILIMGLLYK corresponding to amino acids 1-675 of S21C_HUMAN (SEQ ID NO:1062), which also corresponds to amino acids 1-675 of T23657_P2 (SEQ ID NO:1064), and a second amino acid sequence being at least 70%, optionally at least 80%, preferably at least 85%, more preferably at least 90% and most preferably at least 95% homologous to a polypeptide having the sequence FQLPEVHHSLNVLNRKFQKQTVHNL (SEQ ID NO:1455) corresponding to amino acids 676-700 of T23657_P2 (SEQ ID NO:1064), wherein said first amino acid sequence and second amino acid sequence are contiguous and in a sequential order.

According to preferred embodiments of the present invention, there is provided an isolated polypeptide encoding for a tail of T23657_P2 (SEQ ID NO:1064), comprising a polypeptide being at least 70%, optionally at least about 80%, preferably at least about 85%, more preferably at least about 90% and most preferably at least about 95% homologous to the sequence FQLPEVHHSLNVLNRKFQKQTVHNL (SEQ ID NO:1455) in T23657_P2 (SEQ ID NO:1064).

According to preferred embodiments of the present invention, there is provided an isolated chimeric polypeptide encoding for T23657_P3 (SEQ ID NO:1065), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARGT HEVRYVSAGQSVACGWWAFAPPCLQVLNTPKGILFFLCAAAFLQGMTVNGFINTVITSLERRYDLHSYQSG LIASSYDIAACLCLTFVSYFGGSGHKPRWLGWGVLLMGTGSLVFALPHFTAGRYEVELDAGVRTCPANPGA VCADSTSGLSRYQLVFMLGQFLHGVGATPLYTLGVTYLDENVKSSCSPVYIAIFYTAAILGPAAGYLIGGAL LNIYTEMGRRTELTTESPLWVGAWWVGFLGSGAAAFFTAVPILGYPRQLPGSQRYAVMRAAEMHQLKDSS RGEASNPDFGKTIRDLPLSIWLLLKNPTFILLCLAGATEATLITGMSTFSPKFLESQFSLSASEAATLFGYLVVP AGGGGTFLGGFFVNKLRLRGSAVIKFCLFCTVVSLLGILVFSLHCPSVPMAGVTASYGGSLLPEGHLNLTAP CNAACSCQPEHYSPVCGSDGLMYFSLCHAGCPAATETNVDGQKVYRDCSCIPQNLSSGFGHATAGKCTSTC QRKPLLLVFIFVVIFFTFLSSIPALTATLRCVRDPQRSFALGIQWIVVRILGGIPGPIAFGWVIDKACLLWQDQC GQQGSCLVYQNSAMSRYILIMGLLYK corresponding to amino acids 1-675 of S21C_HUMAN (SEQ ID NO:1062), which also corresponds to amino acids 1-675 of T23657_P3 (SEQ ID NO:1065), and a second amino acid sequence being at least 70%, optionally at least 80%, preferably at least 85%, more preferably at least 90% and most preferably at least 95% homologous to a polypeptide having the sequence TIKHKAF (SEQ ID NO:1456) corresponding to amino acids 676-682 of T23657_P3 (SEQ ID NO:1065), wherein said first amino acid sequence and second amino acid sequence are contiguous and in a sequential order.

According to preferred embodiments of the present invention, there is provided an isolated polypeptide encoding for a tail of T23657_P3 (SEQ ID NO:1065), comprising a polypeptide being at least 70%, optionally at least about 80%, preferably at least about 85%, more preferably at least about 90% and most preferably at least about 95% homologous to the sequence TIKHKAF (SEQ ID NO:1456) in T23657_P3 (SEQ ID NO:1065).

According to preferred embodiments of the present invention, there is provided an isolated chimeric polypeptide encoding for T23657_P4 (SEQ ID NO:1066), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARGT HEVRYVSAGQSVACGWWAFAPPCLQVLNTPKGILFFLCAAAFLQGMTVNGFINTVITSLERRYDLHSYQSG LIASSYDIAACLCLTFVSYFGGSGHKPRWLGWGVLLMGTGSLVFALPHFTAGRYEVELDAGVRTCPANPGA VCADSTSGLSRYQLVFMLGQFLHGVGATPLYTLGVTYLDENVKSSCSPVYIAIFYTAAILGPAAGYLIGGAL LNIYTEMGRRTELTTESPLWVGAWWVGFLGSGAAAFFTAVPILGYPRQLPGSQRYAVMRAAEMHQLKDSS RGEASNPDFGKTIRDLPLSIWLLLKNPTFILLCLAGATEATLITGMSTFSPKFLESQFSLSASEAATLFGYLVVP AGGGGTFLGGFFVNKLRLRGSAVIKFCLFCTVVSLLGILVFSLHCPSVPMAGVTASYGGSLLPEGHLNLTAP CNAACSCQPEHYSPVCGSDGLMYFSLCHAGCPAATETNVDGQKVYRDCSCIPQNLSSGFGHATAGKCTSTC QRKPLLLVFIFVVIFFTFLSSIPALTATLRCVRDPQRSFALGIQWIVVRIL corresponding to amino acids 1-625 of S21C_HUMAN (SEQ ID NO:1062), which also corresponds to amino acids 1-625 of T23657_P4 (SEQ ID NO:1066), a second amino acid sequence being at least 70%, optionally at least 80%, preferably at least 85%, more preferably at least 90% and most preferably at least 95% homologous to a polypeptide having the sequence GTVQCEEAMVSCTVCSLHKGM corresponding to amino acids 626-646 of T23657_P4 (SEQ ID NO:1066), a third amino acid sequence being at least 90% homologous to GGIPGPIAFGWVIDKACLLWQDQCGQQGSCLVYQNSAMSRYILIMGLLYK corresponding to amino acids 626-675 of S21C_HUMAN (SEQ ID NO:1062), which also corresponds to amino acids 647-696 of T23657_P4 (SEQ ID NO:1066), and a fourth amino acid sequence being at least 70%, optionally at least 80%, preferably at least 85%, more preferably at least 90% and most preferably at least 95% homologous to a polypeptide having the sequence TIKHKAF (SEQ ID NO:1456) corresponding to amino acids 697-703 of T23657_P4 (SEQ ID NO:1066), wherein said first amino acid sequence, second amino acid sequence, third amino acid sequence and fourth amino acid sequence are contiguous and in a sequential order.

According to preferred embodiments of the present invention, there is provided an isolated polypeptide encoding for an edge portion of T23657_P4 (SEQ ID NO:1066), comprising an amino acid sequence being at least 70%, optionally at least about 80%, preferably at least about 85%, more preferably at least about 90% and most preferably at least about 95% homologous to the sequence encoding for GTVQCEEAMVSCTVCSLHKGM, corresponding to T23657_P4 (SEQ ID NO:1066).

According to preferred embodiments of the present invention, there is provided an isolated polypeptide encoding for a tail of T23657_P4 (SEQ ID NO:1066), comprising a polypeptide being at least 70%, optionally at least about 80%, preferably at least about 85%, more preferably at least about 90% and most preferably at least about 95% homologous to the sequence TIKHKAF (SEQ ID NO:1456) in T23657_P4 (SEQ ID NO:1066).

According to preferred embodiments of the present invention, there is provided an isolated chimeric polypeptide encoding for T23657_P5 (SEQ ID NO:1067), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARGT HEVRYVSAGQSVACGWWAFAPPCLQVLNTPKGILFFLCAAAFLQGMTVNGFINTVITSLERRYDLHSYQSG LIASSYDIAACLCLTFVSYFGGSGHKPRWLGWGVLLMGTGSLVFALPHFTAGRYEVELDAGVRTCPANPGA VCADSTSGLSRYQLVFMLGQFLHGVGATPLYTLGVTYLDENVKSSCSPVYIAIFYTAAILGPAAGYLIGGAL LNIYTEMGRRTELTTESPLWVGAWWVGFLGSGAAAFFTAVPILGYPRQLPGSQRYAVMRAAEMHQLKDSS RGEASNPDFGKTIRDLPLSIWLLLKNPTFILLCLAGATEATLITGMSTFSPKFLESQFSLSASEAATLFGYLVVP AGGGGTFLGGFFVNKLRLRGSAVIKFCLFCTVVSLLGILVFSLHCPSVPMAGVTASYGGSLLPEGHLNLTAP CNAACSCQPEHYSPVCGSDGLMYFSLCHAGCPAATETNVDGQKVYRDCSCIPQNLSSGFGHATAGKCTSTC QRKPLLLVFIFVVIFFTFLSSIPALTATLR corresponding to amino acids 1-604 of S21C_HUMAN (SEQ ID NO:1062), which also corresponds to amino acids 1-604 of T23657_P5 (SEQ ID NO:1067).

According to preferred embodiments of the present invention, there is provided an isolated chimeric polypeptide encoding for T23657_P6 (SEQ ID NO:1068), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARGT HEVRYVSAGQSVACGWWAFAPPCLQVLNTPKGILFFLCAAAFLQGMTVNGFINTVITSLERRYDLHSYQSG LIASSYDIAACLCLTFVSYFGGSGHKPRWLGWGVLLMGTGSLVFALPHFTAGRYEVELDAGVRTCPANPGA VCADSTSGLSRYQLVFMLGQFLHGVGATPLYTLGVTYLDENVKSSCSPVYIAIFYTAAILGPAAGYLIGGAL LNIYTEMGRRTELTTESPLWVGAWWVGFLGSGAAAFFTAVPILGYPRQLPGSQRYAVMRAAEMHQLKDSS RGEASNPDFGKTIRDLPLSIWLLLKNPTFILLCLAGATEATLITGMSTFSPKFLESQFSLSASEAATLFGYLVVP AGGGGTFLGGFFVNKLRLRGSAVIKFCLFCTVVSLLGILVFSLHCPSVPMAGVTASYGGSLLPEGHLNLTAP CNAACSCQPEHYSPVCGSDGLMYFSLCHAGCPAATETNVDGQKV corresponding to amino acids 1-547 of S21C_HUMAN (SEQ ID NO:1062), which also corresponds to amino acids 1-574 of T23657_P6 (SEQ ID NO:1068), and a second amino acid sequence being at least 70%, optionally at least 80%, preferably at least 85%, more preferably at least 90% and most preferably at least 95% homologous to a polypeptide having the sequence SGAAAYRPCPPLDPGKGPPCLPLVIGAIVGLPRCTETVAVSLRIFPLVLAMPLQGNALQLVRESPSFWFSYSL (SEQ ID NO:1458) corresponding to amino acids 548-620 of T23657_P6 (SEQ ID NO:1068), wherein said first amino acid sequence and second amino acid sequence are contiguous and in a sequential order.

According to preferred embodiments of the present invention, there is provided an isolated polypeptide encoding for a tail of T23657_P6 (SEQ ID NO:1068), comprising a polypeptide being at least 70%, optionally at least about 80%, preferably at least about 85%, more preferably at least about 90% and most preferably at least about 95% homologous to the sequence SGAAAYRPCPPLDPGKGPPCLPLVIGAIVGLPRCTETVAVSLRIFPLVLAMPLQGNALQLVRESPSFWFSYSL (SEQ ID NO:1458) in T23657_P6 (SEQ ID NO:1068).

According to preferred embodiments of the present invention, there is provided an isolated chimeric polypeptide encoding for T23657_P7 (SEQ ID NO:1069), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARGT HEVRYVSAGQSVACGWWAFAPPCLQVLNTPKGILFFLCAAAFLQGMTVNGFINTVITSLERRYDLHSYQSG LIASSYDIAACLCLTFVSYFGGSGHKPRWLGWGVLLMGTGSLVFALPHFTAGRYEVELDAGVRTCPANPGA VCADSTSGLSRYQLVFMLGQFLHGVGATPLYTLGVTYLDENVKSSCSPVYIAIFYTAAILGPAAGYLIGGAL LNIYTEMGRRTELTTESPLWVGAWWVGFLGSGAAAFFTAVPILGYPRQLPGSQRYAVMRAAEMHQLKDSS RGEASNPDFGKTIRDLPLSIWLLLKNPTFILLCLAGATEATLITGMSTFSPKFLESQFSLSASEAATLFGYLVVP AGGGGTFLGGFFVNKLRLRGSAVIKFCLFCTVVSLLGILVFSLHCPSVPMAGVTASYGGSLLPEGHLNLTAP CNAACSCQPEHYSPVCGSDGLMYFSLCHAGCPAATETNVDGQK corresponding to amino acids 1-546 of S21C_HUMAN (SEQ ID NO:1062), which also corresponds to amino acids 1-546 of T23657_P7 (SEQ ID NO:1069), and a second amino acid sequence being at least 70%, optionally at least 80%, preferably at least 85%, more preferably at least 90% and most preferably at least 95% homologous to a polypeptide having the sequence MCP corresponding to amino acids 547-549 of T23657_P7 (SEQ ID NO:1069), wherein said first amino acid sequence and second amino acid sequence are contiguous and in a sequential order.

According to preferred embodiments of the present invention, there is provided an isolated chimeric polypeptide encoding for T23657_P8 (SEQ ID NO:1070), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARGT HEVRYVSAGQSVACGWWAFAPPCLQVLNTPKGILFFLCAAAFLQGMTVNGFINTVITSLERRYDLHSYQSG LIASSYDIAACLCLTFVSYFGGSGHKPRWLGWGVLLMGTGSLVFALPHFTAGRYEVELDAGVRTCPANPGA VCADSTSGLSRYQLVFMLGQFLHGVGATPLYTLGVTYLDENVKSSCSPVYIAIFYTAAILGPAAGYLIGGAL LNIYTEMGRRTELTTESPLWVGAWWVGFLGSGAAAFFTAVPILGYPRQLPGSQRYAVMRAAEMHQLKDSS RGEASNPDFGKTIRDLPLSIWLLLKNPTFILLCLAGATEATLITGMSTFSPKFLESQFSLSASEAATLFGYLVVP AGGGGTFLGGFFVNKLRLRGSAVIKFCLFCTVVSLLGILVFSLHCPSVPMAGVTASYGGSLLPEGHLNLTAP CNAACSCQPEHYSPVCGSDGLMYFSLCHAGCPAATETNVDGQK corresponding to amino acids 1-546 of S21C_HUMAN (SEQ ID NO:1062), which also corresponds to amino acids 1-546 of T23657_P8 (SEQ ID NO:1070), and a second amino acid sequence being at least 70%, optionally at least 80%, preferably at least 85%, more preferably at least 90% and most preferably at least 95% homologous to a polypeptide having the sequence QHSCTNGNSTMCP (SEQ ID NO:1459) corresponding to amino acids 547-559 of T23657_P8 (SEQ ID NO:1070), wherein said first amino acid sequence and second amino acid sequence are contiguous and in a sequential order.

According to preferred embodiments of the present invention, there is provided an isolated polypeptide encoding for a tail of T23657_P8 (SEQ ID NO:1070), comprising a polypeptide being at least 70%, optionally at least about 80%, preferably at least about 85%, more preferably at least about 90% and most preferably at least about 95% homologous to the sequence QHSCTNGNSTMCP (SEQ ID NO:1459) in T23657_P8 (SEQ ID NO:1070).

According to preferred embodiments of the present invention, there is provided an isolated chimeric polypeptide encoding for T23657_P10 (SEQ ID NO:1072), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARGT HEVRYVSAGQSVACGWWAFAPPCLQVLNTPKGILFFLCAAAFLQGMTVNGFINTVITSLERRYDLHSYQSG LIASSYDIAACLCLTFVSYFGGSGHKPRWLGWGVLLMGTGSLVFALPHFTAGRYEVELDAGVRTCPANPGA VCADSTSGLSRYQLVFMLGQFLHGVGATPLYTLGVTYLDENVKSSCSPVYIAIFYTAAILGPAAGYLIGGAL LNIYTEMGRRTELTTESPLWVGAWWVGFLGSGAAAFFTAVPILGYPRQLPGSQRYAVMRAAEMHQLKDSS RGEASNPDFGKTIRDLPLSIWLLLKNPTFILLCLAGATEATLITGMSTFSPKFLESQFSLSASEAATLFGYLVVP AGGGGTFLGGFFVNKLRLRGSAVIKFCLFCTVVSLLGILVFSLHCPSVPMAGVTASYGGSLLPEGHLNLTAP CNAACSCQPEHYSPVCGSDGLMYFSLCHAGCPAATETNVDGQKVYRDCSCIPQNLSSGFGHATAGKCTSTC QRKPLLLVFIFVVIFFTFLSSIPALTATLRCVRDPQRSFALGIQWIVVRIL corresponding to amino acids 1-625 of S21C_HUMAN (SEQ ID NO:1062), which also corresponds to amino acids 1-625 of T23657_P10 (SEQ ID NO:1072), a second amino acid sequence being at least 70%, optionally at least 80%, preferably at least 85%, more preferably at least 90% and most preferably at least 95% homologous to a polypeptide having the sequence GTVQCEEAMVSCTVCSLHKGM corresponding to amino acids 626-646 of T23657_P10 (SEQ ID NO:1072), and a third amino acid sequence being at least 90% homologous to GGIPGPIAFGWVIDKACLLWQDQCGQQGSCLVYQNSAMSRYILIMGLLYKVLGVLFFAIACFLYKPLSESSD GLETCLPSQSSAPDSATDSQLQSSV corresponding to amino acids 626-722 of S21C_HUMAN (SEQ ID NO:1062), which also corresponds to amino acids 647-743 of T23657_P10 (SEQ ID NO:1072), wherein said first amino acid sequence, second amino acid sequence and third amino acid sequence are contiguous and in a sequential order.

According to preferred embodiments of the present invention, there is provided an isolated polypeptide encoding for an edge portion of T23657_P10 (SEQ ID NO:1072), comprising an amino acid sequence being at least 70%, optionally at least about 80%, preferably at least about 85%, more preferably at least about 90% and most preferably at least about 95% homologous to the sequence encoding for GTVQCEEAMVSCTVCSLHKGM, corresponding to T23657_P10 (SEQ ID NO:1072).

According to preferred embodiments of the present invention, there is provided an isolated chimeric polypeptide encoding for T23657_P11 (SEQ ID NO:1073), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARGT HEVRYVSAGQSVACGWWAFAPPCLQVLNTPKGILFFLCAAAFLQGMTVNGFINTVITSLERRYDLHSYQSG LIASSYDIAACLCLTFVSYFGGSGHKPRWLGWGVLLMGTGSLVFALPHFTAGRYEVELDAGVRTCPANPGA VCADSTSGLSRYQLVFMLGQFLHGVGATPLYTLGVTYLDENVKSSCSPVYIAIFYTAAILGPAAGYLIGGAL LNIYTEMGRRTELTTESPLWVGAWWVGFLGSGAAAFFTAVPILGYPRQLPGSQRYAVMRAAEMHQLKDSS RGEASNPDFGKTIRDLPLSIWLLLKNPTFILLCLAGATEATLITGMSTFSPKFLESQFSLSASEAATLF corresponding to amino acids 1-425 of S21C_HUMAN (SEQ ID NO:1062), which also corresponds to amino acids 1-425 of T23657_P11 (SEQ ID NO:1073), and a second amino acid sequence being at least 70%, optionally at least 80%, preferably at least 85%, more preferably at least 90% and most preferably at least 95% homologous to a polypeptide having the sequence ASCPKAT (SEQ ID NO:1460) corresponding to amino acids 426-432 of T23657_P11 (SEQ ID NO:1073), wherein said first amino acid sequence and second amino acid sequence are contiguous and in a sequential order.

According to preferred embodiments of the present invention, there is provided an isolated polypeptide encoding for a tail of T23657_P11 (SEQ ID NO:1073), comprising a polypeptide being at least 70%, optionally at least about 80%, preferably at least about 85%, more preferably at least about 90% and most preferably at least about 95% homologous to the sequence ASCPKAT (SEQ ID NO:1460) in T23657_P11 (SEQ ID NO:1073).

According to preferred embodiments of the present invention, there is provided an isolated chimeric polypeptide encoding for T23657_P12 (SEQ ID NO:1074), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARGT HEVRYVSAGQSVACGWWAFAPPCLQVLNTPKGILFFLCAAAFLQGMTVNGFINTVITSLERRYDLHSYQSG LIASSYDIAACLCLTFVSYFGGSGHKPRWLGWGVLLMGTGSLVFALPHFTAGRYEVELDAGVRTCPANPGA VCADSTSGLSRYQLVFMLGQFLHGVGATPLYTLGVTYLDENVKSSCSPVYIAIFYTAAILGPAAGYLIGGAL LNIYTEMGRRTELTTESPLWVGAWWVGFLGSGAAAFFTAVPILGYPRQLPGSQRYAVMRAAEMHQLKDSS RGEASNPDFGKTIRDLPLSIWLLLKNPTFILLCLAGATEATLITGMSTFSPKFLESQFSLSASEAATLFGYLVVP AGGGGTFLGGFFVNKLRLRGSAVIKFCLFCTVVSLLGILVFSLHCPSVPMAGVTASYGGSLLPEGHLNLTAP CNAACSCQPEHYSPVCGSDGLMYFSLCHAGCPAATETNVDGQKVYRDCSCIPQNLSSGFGHATAGKCTSTC QRKPLLLVFIFVVIFFTFLSSIPALTATLRCVRDPQRSFALGIQWUVRILGGIPGPIAFGWVIDKACLLWQDQC GQQGSCLVYQNSAMSRYILIMGLLYK corresponding to amino acids 1-675 of S21C_HUMAN (SEQ ID NO:1062), which also corresponds to amino acids 1-675 of T23657_P12 (SEQ ID NO:1074), and a second amino acid sequence being at least 70%, optionally at least 80%, preferably at least 85%, more preferably at least 90% and most preferably at least 95% homologous to a polypeptide having the sequence EEENEFRRL (SEQ ID NO:1461) corresponding to amino acids 676-684 of T23657_P12 (SEQ ID NO:1074), wherein said first amino acid sequence and second amino acid sequence are contiguous and in a sequential order.

According to preferred embodiments of the present invention, there is provided an isolated polypeptide encoding for a tail of T23657_P12 (SEQ ID NO:1074), comprising a polypeptide being at least 70%, optionally at least about 80%, preferably at least about 85%, more preferably at least about 90% and most preferably at least about 95% homologous to the sequence EEENEFRRL (SEQ ID NO:1461) in T23657_P12 (SEQ ID NO:1074).

According to preferred embodiments of the present invention, there is provided an isolated chimeric polypeptide encoding for T23657_P16 (SEQ ID NO:1075), comprising a first amino acid sequence being at least 70%, optionally at least 80%, preferably at least 85%, more preferably at least 90% and most preferably at least 95% homologous to a polypeptide having the sequence MGTSPMADPVPAGRQHGSGLDPTTRLSPLC (SEQ ID NO:1462) corresponding to amino acids 1-30 of T23657_P16 (SEQ ID NO:1075), and a second amino acid sequence being at least 90% homologous to SLLPEGHLNLTAPCNAACSCQPEHYSPVCGSDGLMYFSLCHAGCPAATETNVDGQKVYRDCSCIPQNLSSG FGHATAGKCTSTCQRKPLLLVFIFVVIFFTFLSSIPALTATLRCVRDPQRSFALGIQWIVVRILGGIPGPIAFGW VIDKACLLWQDQCGQQGSCLVYQNSAMSRYILIMGLLYKVLGVLFFAIACFLYKPLSESSDGLETCLPSQSS APDSATDSQLQSSV corresponding to amino acids 491-722 of S21C_HUMAN (SEQ ID NO:1062), which also corresponds to amino acids 31-262 of T23657_P16 (SEQ ID NO:1075), wherein said first amino acid sequence and second amino acid sequence are contiguous and in a sequential order.

According to preferred embodiments of the present invention, there is provided an isolated polypeptide encoding for a head of T23657_P16 (SEQ ID NO:1075), comprising a polypeptide being at least 70%, optionally at least about 80%, preferably at least about 85%, more preferably at least about 90% and most preferably at least about 95% homologous to the sequence MGTSPMADPVPAGRQHGSGLDPTTRLSPLC (SEQ ID NO:1462) of T23657_P16 (SEQ ID NO:1075).

According to preferred embodiments of the present invention, there is provided an isolated chimeric polypeptide encoding for T23657_P17 (SEQ ID NO:1076), comprising a first amino acid sequence being at least 90% homologous to MYFSLCHAGCPAATETNVDGQKVYRDCSCIPQNLSSGFGHATAGKCTSTCQRKPLLLVFIFVVIFFTFLSSIP ALTATLRCVRDPQRSFALGIQWIVVRILGGIPGPIAFGWVIDKACLLWQDQCGQQGSCLVYQNSAMSRYILI MGLLYKVLGVLFFAIACFLYKPLSESSDGLETCLPSQSSAPDSATDSQLQSSV corresponding to amino acids 525-722 of S21C_HUMAN (SEQ ID NO:1062), which also corresponds to amino acids 1-198 of T23657_P17 (SEQ ID NO:1076).

According to preferred embodiments of the present invention, there is provided an isolated chimeric polypeptide encoding for T23657_P21 (SEQ ID NO:1078), comprising a first amino acid sequence being at least 70%, optionally at least 80%, preferably at least 85%, more preferably at least 90% and most preferably at least 95% homologous to a polypeptide having the sequence MWTAR (SEQ ID NO:1463) corresponding to amino acids 1-5 of T23657_P21 (SEQ ID NO:1078), and a second amino acid sequence being at least 90% homologous to RCVRDPQRSFALGIQWIVVRILGGIPGPIAFGWVIDKACLLWQDQCGQQGSCLVYQNSAMSRYILIMGLLYK VLGVLFFAIACFLYKPLSESSDGLETCLPSQSSAPDSATDSQLQSSV corresponding to amino acids 604-722 of S21C HUMAN (SEQ ID NO:1062), which also corresponds to amino acids 6-124 of T23657_P21 (SEQ ID NO:1078), wherein said first amino acid sequence and second amino acid sequence are contiguous and in a sequential order.

According to preferred embodiments of the present invention, there is provided an isolated polypeptide encoding for a head of T23657_P21 (SEQ ID NO:1078), comprising a polypeptide being at least 70%, optionally at least about 80%, preferably at least about 85%, more preferably at least about 90% and most preferably at least about 95% homologous to the sequence MWTAR (SEQ ID NO:1463) of T23657_P21 (SEQ ID NO:1078).

According to preferred embodiments of the present invention, there is provided an isolated chimeric polypeptide encoding for T23657_P23 (SEQ ID NO:1080), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARGT HEVRYVSAGQSVACGWWAFAPPCLQVLNTPKGILFFLCAAAFLQGMTVNGFINTVITSLERRYDLHSYQSG LIASSYDIAACLCLTFVSYFGGSGHKPRWLGWGVLLMGTGSLVFALPHFTAGRYEVELDAGVRTCPANPGA VCADSTSGLSRYQLVFMLGQFLHGVGATPLYTLGVTYLDENVKSSCSPVYIAIFYTAAILGPAAGYLIGGAL LNIYTEMGRRTELTTESPLWVGAWWVGFLGSGAAAFFTAVPILGYPRQLPGSQRYAVMRAAEMHQLKDSS RGEASNPDFGKTIRDLPLSIWLLLKNPTFILLCLAGATEATLITGMSTFSPKFLESQFSLSASEAATLFGYLVVP AGGGGTFLGGFFVNKLRLRGSAVIKFCLFCTVVSLLGILVFSLHCPSVPMAGVTASYGGSLLPEGHLNLTAP CNAACSCQPEHYSPVCGSDGLMYFSLCHAGCPAATETNVDGQKV corresponding to amino acids 1-547 of S21C_HUMAN (SEQ ID NO:1062), which also corresponds to amino acids 1-547 of T23657_P23 (SEQ ID NO:1080), and a second amino acid sequence being at least 70%, optionally at least 80%, preferably at least 85%, more preferably at least 90% and most preferably at least 95% homologous to a polypeptide having the sequence SGAAAYRPCPPLDPGKGPPCLPLVIGAIVGLPRCTETVAVSLRIFPLVLAMHCREMHFNLSEKAPPSGFHIRC NFLYIPQQHSCTNGNSTVSWGRVCACPELSLQHPEAELCRS (SEQ ID NO:1464) corresponding to amino acids 548-661 of T23657_P23 (SEQ ID NO:1080), wherein said first amino acid sequence and second amino acid sequence are contiguous and in a sequential order.

According to preferred embodiments of the present invention, there is provided an isolated polypeptide encoding for a tail of T23657_P23 (SEQ ID NO:1080), comprising a polypeptide being at least 70%, optionally at least about 80%, preferably at least about 85%, more preferably at least about 90% and most preferably at least about 95% homologous to the sequence SGAAAYRPCPPLDPGKGPPCLPLVIGAIVGLPRCTETVAVSLRIFPLVLAMHCREMHFNLSEKAPPSGFHIRC NFLYIPQQHSCTNGNSTVSWGRVCACPELSLQHPEAELCRS (SEQ ID NO:1464) in T23657_P23 (SEQ ID NO:1080).

According to preferred embodiments of the present invention, there is provided an isolated chimeric polypeptide encoding for R30650_PEA_{—}2_P4 (SEQ ID NO:991), comprising a first amino acid sequence being at least 90% homologous to MYLHIGEEIDGVDMRAEVGLLSRNIIVMGEMEDKCYPYRNHICNFFDFDTFGGHIKFALGFKAAHLEGTELK HMGQQLVGQYPIHFHLAGDVDERGGYDPPTYIRDLSIHHTFSRCVTVHGSNGLLIKDVVGYNSLGHCFFTE DGPEERNTFDHCLGLLVKSGTLLPSDRDSKMCKMITEDSYPGYIPKPRQDCNAVSTFWMANPNNNLINCAA AGSEETGFWFIFHHVPTGPSVGMYSPGYSEHIPLGKFYNNRAHSNYRAGMIIDNGVKTTEASAKDKRPFLSII SARYSPHQDADPLKPREPAIIRHFIAYKNQDHGAWLRGGDVWLDSCRFADNGIGLTLASGGTFPYDDGSKQ EIKNSLFVGESGNVGTEMMDNRIWGPGGLDHSGRTLPIGQNFPIRGIQLYDGPINIQNCTFRKFVALEGRHTS ALAFRLNNAWQSCPHNNVTGIAFEDVPITSRVFFGEPGPWFNQLDMDGDKTSVFHDVDGSVSEYPGSYLTK NDNWLVRHPDCINVPDWRGAICSGCYAQMYIQAYKTSNLRMKIIKNDFPSHPLYLEGALTRSTHYQQYQPV VTLQKGYTIHWDQTAPAELAIWLINFNKGDWIRVGLCYPRGTTFSILSDVHNRLLKQTSKTGVFVRTLQMD KVEQSYPGRSHYYWDEDSGLLFLKLKAQNEREKFAFCSMKGCERIKIKALIPKNAGVSDCTATAYPKFTER AVVDVPMPKKLFGSQLKTKDHFLEVKMESSKQHFFHLWNDFAYIEVDGKKYPSSEDGIQVVVIDGNQGRV VSHTSFRNSILQGIPWQLFNYVATIPDNSIVLMASKGRYVSRGPWTRVLEKLGADRGLKLKEQMAFVGFKG SFRPIWVTLDTEDHKAKIFQVVPIPVVKKKKL corresponding to amino acids 126-1013 of Q9ULM1 (SEQ ID NO:989), which also corresponds to amino acids 1-888 of R30650_PEA_{—}2_P4 (SEQ ID NO:991).

According to preferred embodiments of the present invention, there is provided an isolated chimeric polypeptide encoding for R30650_PEA_{—}2_P4 (SEQ ID NO:991), comprising a first amino acid sequence being at least 90% homologous to MYLHIGEEIDGVDMRAEVGLLSRNIWMGEMEDKCYPYRNHICNFFDFDTFGGHIKFALGFKAAHLEGTELK HMGQQLVGQYPIHFHLAGDVDERGGYDPPTYIRDLSIHHTFSRCVTVHGSNGLLIKDVVGYNSLGHCFFTE DGPEERNTFDHCLGLLVKSGTLLPSDRDSKMCKMITEDSYPGYIPKPRQDCNAVSTFWMANPNNNLINCAA AGSEETGFWFIFHHVPTGPSVGMYSPGYSEHIPLGKFYNNRAHSNYRAGMIIDNGVKTTEASAKDKRPFLSII SARYSPHQDADPLKPREPAIIRHFIAYKNQDHGAWLRGGDVWLDSCRFADNGIGLTLASGGTFPYDDGSKQ EIKNSLFVGESGNVGTEMMDNRIWGPGGLDHSGRTLPIGQNFPIRGIQLYDGPINIQNCTFRKFVALEGRHTS ALAFRLNNAWQSCPHNNVTGIAFEDVPITSRVFFGEPGPWFNQLDMDGDKTSVFHDVDGSVSEYPGSYLTK ND corresponding to amino acids 474-977 of Q8WUJ3 (SEQ ID NO:987), which also corresponds to amino acids 1-504 of R30650_PEA_{—}2_P4 (SEQ ID NO:991), and a second amino acid sequence being at least 70%, optionally at least 80%, preferably at least 85%, more preferably at least 90% and most preferably at least 95% homologous to a polypeptide having the sequence NWLVRHPDCINVPDWRGAICSGCYAQMYIQAYKTSNLRMKIIKNDFPSHPLYLEGALTRSTHYQQYQPVVT LQKGYTIHWDQTAPAELAIWLINFNKGDWIRVGLCYPRGTTFSILSDVHNRLLKQTSKTGVFVRTLQMDKV EQSYPGRSHYYWDEDSGLLFLKLKAQNEREKFAFCSMKGCERIKIKALIPKNAGVSDCTATAYPKFTERAV VDVPMPKKLFGSQLKTKDHFLEVKMESSKQHFFHLWNDFAYIEVDGKKYPSSEDGIQVVVIDGNQGRVVS HTSFRNSILQGIPWQLFNYVATIPDNSIVLMASKGRYVSRGPWTRVLEKLGADRGLKLKEQMAFVGFKGSF RPIWVTLDTEDHKAKIFQVVPIPVVKKKKL (SEQ ID NO:1465) corresponding to amino acids 505-888 of R30650_PEA_{—}2_P4 (SEQ ID NO:991), wherein said first amino acid sequence and second amino acid sequence are contiguous and in a sequential order.

According to preferred embodiments of the present invention, there is provided an isolated polypeptide encoding for a tail of R30650_PEA_{—}2_P4 (SEQ ID NO:991), comprising a polypeptide being at least 70%, optionally at least about 80%, preferably at least about 85%, more preferably at least about 90% and most preferably at least about 95% homologous to the sequence NWLVRHPDCINVPDWRGAICSGCYAQMYIQAYKTSNLRMKIIKNDFPSHPLYLEGALTRSTHYQQYQPVVT LQKGYTIHWDQTAPAELAIWLINFNKGDWIRVGLCYPRGTTFSILSDVHNRLLKQTSKTGVFVRTLQMDKV EQSYPGRSHYYWDEDSGLLFLKLKAQNEREKFAFCSMKGCERIKIKALIPKNAGVSDCTATAYPKFTERAV VDVPMPKKLFGSQLKTKDHFLEVKMESSKQHFFHLWNDFAYIEVDGKKYPSSEDGIQVVVIDGNQGRVVS HTSFRNSILQGIPWQLFNYVATIPDNSIVLMASKGRYVSRGPWTRVLEKLGADRGLKLKEQMAFVGFKGSF RPIWVTLDTEDHKAKIFQVVPIPVVKKKKL (SEQ ID NO:1465) in R30650_PEA_{—}2_P4 (SEQ ID NO:991).

According to preferred embodiments of the present invention, there is provided an isolated chimeric polypeptide encoding for R30650_PEA_{—}2_P4 (SEQ ID NO:991), comprising a first amino acid sequence being at least 70%, optionally at least 80%, preferably at least 85%, more preferably at least 90% and most preferably at least 95% homologous to a polypeptide having the sequence MYLHIGEEIDGVDMRAEVGLLSRNIIVMGEMEDKCYPYRNHICNFFDFDTFGGHIKFALGFKAAHLEGTELK HMGQQLVGQYPIHFHLAGD (SEQ ID NO:1466) corresponding to amino acids 1-91 of R30650_PEA_{—}2_P4 (SEQ ID NO:991), and a second amino acid sequence being at least 90% homologous to VDERGGYDPPTYIRDLSIHHTFSRCVTVHGSNGLLIKDVVGYNSLGHCFFTEDGPEERNTFDHCLGLLVKSG TLLPSDRDSKMCKMITEDSYPGYIPKPRQDCNAVSTFWMANPNNNLINCAAAGSEETGFWFIFHHVPTGPSV GMYSPGYSEHIPLGKFYNNPAHSNYRAGMIIDNGVKTTEASAKDKRPFLSIISARYSPHQDADPLKPREPAII RHFIAYKNQDHGAWLRGGDVWLDSCRFADNGIGLTLASGGTFPYDDGSKQEIKNSLFVGESGNVGTEMMD NRIWGPGGLDHSGRTLPIGQNFPIRGIQLYDGPINIQNCTFRKFVALEGRHTSALAFRLNNAWQSCPHNNVT GIAFEDVPITSRVFFGEPGPWFNQLDMDGDKTSVFHDVDGSVSEYPGSYLTKNDNWLVRHPDCINVPDWRG AICSGCYAQMYIQAYKTSNLRMKIIKNDFPSHPLYLEGALTRSTHYQQYQPVVTLQKGYTIHWDQTAPAEL AIWLINFNKGDWIRVGLCYPRGTTFSILSDVHNRLLKQTSKTGVFVRTLQMDKVEQSYPGRSHYYWDEDSG LLFLKLKAQNEREKFAFCSMKGCERIKIKALIPKNAGVSDCTATAYPKFTERAVVDVPMPKKLFGSQLKTK DHFLEVKMESSKQHFFHLWNDFAYIEVDGKKYPSSEDGIQVVVIDGNQGRVVSHTSFRNSILQGIPWQLFNY VATIPDNSIVLMASKGRYVSRGPWTRVLEKLGADRGLKLKEQMAFVGFKGSFRPIWVTLDTEDHKAKIFQV VPIPVVKKKKL corresponding to amino acids 8-804 of Q9NPN9 (SEQ ID NO:988), which also corresponds to amino acids 92-888 of R30650_PEA_{—}2_P4 (SEQ ID NO:991), wherein said first amino acid sequence and second amino acid sequence are contiguous and in a sequential order.

According to preferred embodiments of the present invention, there is provided an isolated polypeptide encoding for a head of R30650_PEA_{—}2_P4 (SEQ ID NO:991), comprising a polypeptide being at least 70%, optionally at least about 80%, preferably at least about 85%, more preferably at least about 90% and most preferably at least about 95% homologous to the sequence MYLHIGEEIDGVDMRAEVGLLSRNIIVMGEMEDKCYPYRNHICNFFDFDTFGGHIKFALGFKAAHLEGTELK HMGQQLVGQYPIHFHLAGD (SEQ ID NO:1466) of R30650_PEA_{—}2_P4 (SEQ ID NO:991).

According to preferred embodiments of the present invention, there is provided an isolated chimeric polypeptide encoding for R30650_PEA_{—}2_P4 (SEQ ID NO:991), comprising a first amino acid sequence being at least 70%, optionally at least 80%, preferably at least 85%, more preferably at least 90% and most preferably at least 95% homologous to a polypeptide having the sequence MYLHIGEEIDGVDMRAEVGLLSRNIIVMGEMEDKCYPYRNHICNFFDFDTFGGHIKFALGFKAAHLEGTELK HMGQQLVGQYPIHFHLAGDVDERGGYDPPTYIRDLSIHHTFSRCVTVHGSNGLLIKDVVGYNSLGHCFFTE DGPEERNTFDHCLGLLVKSGTLLPSDRDSKMCKMITEDSYPGYIPKPRQDCNAVSTFWMANPNNNLINCAA AGSEETGFWFIFHHVPTGPSVGMYSPGYSEHIPLGKFYNNPAHSNYRAGMIIDNGVKTTEASAKDKRPFLSII SARYSPHQDADPLKPREPAIIRHFIAYKNQDHGAWLRGGDVWLDSCRFADNGIGLTLASGGTFPYDDGSKQ EIKNSLFVGESGNVGTEMMDNRIWGPGGLDH corresponding to amino acids 1-389 of R30650_PEA_{—}2_P4 (SEQ ID NO:991), and a second amino acid sequence being at least 90% homologous to SGRTLPIGQNFPIRGIQLYDGPINIQNCTFRKFVALEGRHTSALAFRLNNAWQSCPHNNVTGIAFEDVPITSRV FFGEPGPWFNQLDMDGDKTSVFHDVDGSVSEYPGSYLTKNDNWLVRHPDCINVPDWRGAICSGCYAQMYI QAYKTSNLRMKIIKNDFPSHPLYLEGALTRSTHYQQYQPVVTLQKGYTIHWDQTAPAELAIWLINFNKGDW IRVGLCYPRGTTFSILSDVHNRLLKQTSKTGVFVRTLQMDKVEQSYPGRSHYYWDEDSGLLFLKLKAQNER EKFAFCSMKGCERIKIKALIPKNAGVSDCTATAYPKFTERAVVDVPMPKKLFGSQLKTKDHFLEVKMESSK QHFFHLWNDFAYIEVDGKKYPSSEDGIQVVVIDGNQGRVVSHTSFRNSILQGIPWQLFNYVATIPDNSIVLM ASKGRYVSRGPWTRVLEKLGADRGLKLKEQMAFVGFKGSFRPIWVTLDTEDHKAKIFQVVPIPVVKKKKL corresponding to amino acids 2-500 of Q9H1K5 (SEQ ID NO:990), which also corresponds to amino acids 390-888 of R30650_PEA_{—}2_P4 (SEQ ID NO:991), wherein said first amino acid sequence and second amino acid sequence are contiguous and in a sequential order.

According to preferred embodiments of the present invention, there is provided an isolated polypeptide encoding for a head of R30650_PEA_{—}2_P4 (SEQ ID NO:991), comprising a polypeptide being at least 70%, optionally at least about 80%, preferably at least about 85%, more preferably at least about 90% and most preferably at least about 95% homologous to the sequence MYLHIGEEIDGVDMRAEVGLLSRNIIVMGEMEDKCYPYRNHICNFFDFDTFGGHIKFALGFKAAHLEGTELK HMGQQLVGQYPIHFHLAGDVDERGGYDPPTYIRDLSIHHTFSRCVTVHGSNGLLIKDVVGYNSLGHCFFTE DGPEERNTFDHCLGLLVKSGTLLPSDRDSKMCKMITEDSYPGYIPKPRQDCNAVSTFWMANPNNNLINCAA AGSEETGFWFIFHHVPTGPSVGMYSPGYSEHIPLGKFYNNRAHSNYRAGMIIDNGVKTTEASAKDKRPFLSII SARYSPHQDADPLKPREPAIIRHFIAYKNQDHGAWLRGGDVWLDSCRFADNGIGLTLASGGTFPYDDGSKQ EIKNSLFVGESGNVGTEMMDNRIWGPGGLDH of R30650_PEA_{—}2_P4 (SEQ ID NO:991).

According to preferred embodiments of the present invention, there is provided an isolated chimeric polypeptide encoding for R30650_PEA_{—}2_P5 (SEQ ID NO:992), comprising a first amino acid sequence being at least 90% homologous to MDGVNLSTEVVYKKGQDYRFACYDRGRACRSYRVRFLCGKPVRPKLTVTIDTNVNSTILNLEDNVQSWKP GDTLVIASTDYSMYQAEEFQVLPCRSCAPNQVKVAGKPMYLHIGEEIDGVDMRAEVGLLSRNIIVMGEMED KCYPYRNHICNFFDFDTFGGHIKFALGFKAAHLEGTELKHMGQQLVGQYPIHFHLAGDVDERGGYDPPTYI RDLSIHHTFSRCVTVHGSNGLLIKDVVGYNSLGHCFFTEDGPEERNTFDHCLGLLVKSGTLLPSDRDSKMCK MITEDSYPGYIPKPRQDCNAVSTFWMANPNNNLINCAAAGSEETGFWFIFHHVPTGPSVGMYSPGYSEHIPL GKFYNNRAHSNYRAGMIIDNGVKTTEASAKDKRPFLSIISARYSPHQDADPLKPREPAIIRHFIAYKNQDHGA WLRGGDVWLDSCRFADNGIGLTLASGGTFPYDDGSKQEIKNSLFVGESGNVGTEMMDNRIWGPGGLDHSG RTLPIGQNFPIRGIQLYDGPINIQNCTFRKFVALEGRHTSALAFRLNNAWQSCPHNNVTGIAFEDVPITSRVFF GEPGPWFNQLDMDGDKTSVFHDVDGSVSEYPGSYLTKNDNWLVRHPDCINVPDWRGAICSGCYAQMYIQ AYKTSNLRMKIIKNDFPSHPLYLEGALTRSTHYQQYQPVVTLQKGYTIHWDQTAPAELAIWLINFNKGDWI RVGLCYPRGTTFSILSDVHNRLLKQTSKTGVFVRTLQMDKVEQSYPGRSHYYWDEDSGLLFLKLKAQNERE KFAFCSMKGCERIKIKALIPKNAGVSDCTATAYPKFTERAVVDVPMPKKLFGSQLKTKDHFLEVKMESSKQ HFFHLWNDFAYIEVDGKKYPSSEDGIQVVVIDGNQGRVVSHTSFRNSILQGIPWQLFNYVATIPDNSIVLMA SKGRYVSRGPWTRVLEKLGADRGLKLKEQMAFVGFKGSFRPIWVTLDTEDHKAKIFQVVPIPVVKKKKL corresponding to amino acids 18-1013 of Q9ULM1 (SEQ ID NO:989), which also corresponds to amino acids 1-996 of R30650_PEA_{—}2_P5 (SEQ ID NO:992).

According to preferred embodiments of the present invention, there is provided an isolated chimeric polypeptide encoding for R30650_PEA_{—}2_P5 (SEQ ID NO:992), comprising a first amino acid sequence being at least 90% homologous to MDGVNLSTEVVYKKGQDYRFACYDRGRACRSYRVRFLCGKPVRPKLTVTIDTNVNSTILNLEDNVQSWKP GDTLVIASTDYSMYQAEEFQVLPCRSCAPNQVKVAGKPMYLHIGEEIDGVDMRAEVGLLSRNIIVMGEMED KCYPYRNHICNFFDFDTFGGHIKFALGFKAAHLEGTELKHMGQQLVGQYPIHFHLAGDVDERGGYDPPTYI RDLSIHHTFSRCVTVHGSNGLLIKDVVGYNSLGHCFFTEDGPEERNTFDHCLGLLVKSGTLLPSDRDSKMCK MITEDSYPGYIPKPRQDCNAVSTFWMANPNNNLINCAAAGSEETGFWFIFHHVPTGPSVGMYSPGYSEHIPL GKFYNNRAHSNYRAGMIIDNGVKTTEASAKDKRPFLSIISARYSPHQDADPLKPREPAIIRHFIAYKNQDHGA WLRGGDVWLDSCRFADNGIGLTLASGGTFPYDDGSKQEIKNSLFVGESGNVGTEMMDNRIWGPGGLDHSG RTLPIGQNFPIRGIQLYDGPINIQNCTFRKFVALEGRHTSALAFRLNNAWQSCPHNNVTGIAFEDVPITSRVFF GEPGPWFNQLDMDGDKTSVFHDVDGSVSEYPGSYLTKND corresponding to amino acids 366-977 of Q8WUJ3 (SEQ ID NO:987), which also corresponds to amino acids 1-612 of R30650_PEA_{—}2_P5 (SEQ ID NO:992), and a second amino acid sequence being at least 70%, optionally at least 80%, preferably at least 85%, more preferably at least 90% and most preferably at least 95% homologous to a polypeptide having the sequence NWLVRHPDCINVPDWRGAICSGCYAQMYIQAYKTSNLRMKIIKNDFPSHPLYLEGALTRSTHYQQYQPVVT LQKGYTIHWDQTAPAELAIWLINFNKGDWIRVGLCYPRGTTFSILSDVHNRLLKQTSKTGVFVRTLQMDKV EQSYPGRSHYYWDEDSGLLFLKLKAQNEREKFAFCSMKGCERIKIKALIPKNAGVSDCTATAYPKFTERAV VDVPMPKKLFGSQLKTKDHFLEVKMESSKQHFFHLWNDFAYIEVDGKKYPSSEDGIQVVVIDGNQGRVVS HTSFRNSILQGIPWQLFNYVATIPDNSIVLMASKGRYVSRGPWTRVLEKLGADRGLKLKEQMAFVGFKGSF RPIWVTLDTEDHKAKIFQVVPIPVVKKKKL (SEQ ID NO:1465) corresponding to amino acids 613-996 of R30650_PEA_{—}2_P5 (SEQ ID NO:992), wherein said first amino acid sequence and second amino acid sequence are contiguous and in a sequential order.

According to preferred embodiments of the present invention, there is provided an isolated polypeptide encoding for a tail of R30650_PEA_{—}2_P5 (SEQ ID NO:992), comprising a polypeptide being at least 70%, optionally at least about 80%, preferably at least about 85%, more preferably at least about 90% and most preferably at least about 95% homologous to the sequence NWLVRHPDCINVPDWRGAICSGCYAQMYIQAYKTSNLRMKIIKNDFPSHPLYLEGALTRSTHYQQYQPVVT LQKGYTIHWDQTAPAELAIWLINFNKGDWIRVGLCYPRGTTFSILSDVHNRLLKQTSKTGVFVRTLQMDKV EQSYPGRSHYYWDEDSGLLFLKLKAQNEREKFAFCSMKGCERIKIKALIPKNAGVSDCTATAYPKFTERAV VDVPMPKKLFGSQLKTKDHFLEVKMESSKQHFFHLWNDFAYIEVDGKKYPSSEDGIQVVVIDGNQGRVVS HTSFRNSILQGIPWQLFNYVATIPDNSIVLMASKGRYVSRGPWTRVLEKLGADRGLKLKEQMAFVGFKGSF RPIWVTLDTEDHKAKIFQVVPIPVVKKKKL (SEQ ID NO:1465) in R30650_PEA_{—}2_P5 (SEQ ID NO:992).

According to preferred embodiments of the present invention, there is provided an isolated chimeric polypeptide encoding for R30650_PEA_{—}2_P5 (SEQ ID NO:992), comprising a first amino acid sequence being at least 70%, optionally at least 80%, preferably at least 85%, more preferably at least 90% and most preferably at least 95% homologous to a polypeptide having the sequence MDGVNLSTEVVYKKGQDYRFACYDRGRACRSYRVRFLCGKPVRPKLTVTIDTNVNSTILNLEDNVQSWKP GDTLVIASTDYSMYQAEEFQVLPCRSCAPNQVKVAGKPMYLHIGEEIDGVDMRAEVGLLSRNIIVMGEMED KCYPYRNHICNFFDFDTFGGHIKFALGFKAAHLEGTELKHMGQQLVGQYPIHFHLAGD (SEQ ID NO:1468) corresponding to amino acids 1-199 of R30650_PEA_{—}2_P5 (SEQ ID NO:992), and a second amino acid sequence being at least 90% homologous to VDERGGYDPPTYIRDLSIHHTFSRCVTVHGSNGLLIKDVVGYNSLGHCFFTEDGPEERNTFDHCLGLLVKSG TLLPSDRDSKMCKMITEDSYPGYIPKPRQDCNAVSTFWMANPNNNLINCAAAGSEETGFWFIFHHVPTGPSV GMYSPGYSEHIPLGKFYNNRAHSNYRAGMIIDNGVKTTEASAKDKRPFLSIISARYSPHQDADPLKPREPAII RHFIAYKNQDHGAWLRGGDVWLDSCRFADNGIGLTLASGGTFPYDDGSKQEIKNSLFVGESGNVGTEMMD NRIWGPGGLDHSGRTLPIGQNFPIRGIQLYDGPINIQNCTFRKFVALEGRHTSALAFRLNNAWQSCPHNNVT GIAFEDVPITSRVFFGEPGPWFNQLDMDGDKTSVFHDVDGSVSEYPGSYLTKNDNWLVRHPDCINVPDWRG AICSGCYAQMYIQAYKTSNLRMKIIKNDFPSHPLYLEGALTRSTHYQQYQPVVTLQKGYTIHWDQTAPAEL AIWLINFNKGDWIRVGLCYPRGTTFSILSDVHNRLLKQTSKTGVFVRTLQMDKVEQSYPGRSHYYWDEDSG LLFLKLKAQNEREKFAFCSMKGCERIKIKALIPKNAGVSDCTATAYPKFTERAVVDVPMPKKLFGSQLKTK DHFLEVKMESSKQHFFHLWNDFAYIEVDGKKYPSSEDGIQVVVIDGNQGRVVSHTSFRNSILQGIPWQLFNY VATIPDNSIVLMASKGRYVSRGPWTRVLEKLGADRGLKLKEQMAFVGFKGSFRPIWVTLDTEDHKAKIFQV VPIPVVKKKKL corresponding to amino acids 8-804 of Q9NPN9 (SEQ ID NO:988), which also corresponds to amino acids 200-996 of R30650_PEA_{—}2_P5 (SEQ ID NO:992), wherein said first amino acid sequence and second amino acid sequence are contiguous and in a sequential order.

According to preferred embodiments of the present invention, there is provided an isolated polypeptide encoding for a head of R30650_PEA_{—}2_P5 (SEQ ID NO:992), comprising a polypeptide being at least 70%, optionally at least about 80%, preferably at least about 85%, more preferably at least about 90% and most preferably at least about 95% homologous to the sequence MDGVNLSTEVVYKKGQDYRFACYDRGRACRSYRVRFLCGKPVRPKLTVTIDTNVNSTILNLEDNVQSWKP GDTLVIASTDYSMYQAEEFQVLPCRSCAPNQVKVAGKPMYLHIGEEIDGVDMRAEVGLLSRNIIVMGEMED KCYPYRNHICNFFDFDTFGGHIKFALGFKAAHLEGTELKHMGQQLVGQYPIHFHLAGD (SEQ ID NO:1468) of R30650_PEA_{—}2_P5 (SEQ ID NO:992).

According to preferred embodiments of the present invention, there is provided an isolated chimeric polypeptide encoding for R30650_PEA_{—}2_P5 (SEQ ID NO:992), comprising a first amino acid sequence being at least 70%, optionally at least 80%, preferably at least 85%, more preferably at least 90% and most preferably at least 95% homologous to a polypeptide having the sequence MDGVNLSTEVVYKKGQDYRFACYDRGPACRSYRVRFLCGKPVRPKLTVTIDTNVNSTILNLEDNVQSWKP GDTLVIASTDYSMYQAEEFQVLPCRSCAPNQVKVAGKPMYLHIGEEIDGVDMRAEVGLLSRNIIVMGEMED KCYPYRNHICNFFDFDTFGGHIKFALGFKAAHLEGTELKHMGQQLVGQYPIHFHLAGDVDERGGYDPPTYI RDLSIHHTFSRCVTVHGSNGLLIKDVVGYNSLGHCFFTEDGPEERNTFDHCLGLLVKSGTLLPSDRDSKMCK MITEDSYPGYIPKPRQDCNAVSTFWMANPNNNLINCAAAGSEETGFWFIFHHVPTGPSVGMYSPGYSEHIPL GKFYNNPAHSNYRAGMIIDNGVKTTEASAKDKRPFLSIISARYSPHQDADPLKPREPAIIRHFIAYKNQDHGA WLRGGDVWLDSCRFADNGIGLTLASGGTFPYDDGSKQEIKNSLFVGESGNVGTEMMDNRIWGPGGLDH corresponding to amino acids 1-497 of R30650_PEA_{—}2_P5 (SEQ ID NO:992), and a second amino acid sequence being at least 90% homologous to SGRTLPIGQNFPIRGIQLYDGPINIQNCTFRKFVALEGRHTSALAFRLNNAWQSCPHNNVTGIAFEDVPITSRV FFGEPGPWFNQLDMDGDKTSVFHDVDGSVSEYPGSYLTKNDNWLVRHPDCINVPDWRGAICSGCYAQMYI QAYKTSNLRMKIIKNDFPSHPLYLEGALTRSTHYQQYQPVVTLQKGYTIHWDQTAPAELAIWLINFNKGDW IRVGLCYPRGTTFSILSDVHNRLLKQTSKTGVFVRTLQMDKVEQSYPGRSHYYWDEDSGLLFLKLKAQNER EKFAFCSMKGCERIKIKALIPKNAGVSDCTATAYPKFTERAVVDVPMPKKLFGSQLKTKDHFLEVKMESSK QHFFHLWNDFAYIEVDGKKYPSSEDGIQVVVIDGNQGRVVSHTSFRNSILQGIPWQLFNYVATIPDNSIVLM ASKGRYVSRGPWTRVLEKLGADRGLKLKEQMAFVGFKGSFRPIWVTLDTEDHKAKIFQVVPIPVVKKKKL corresponding to amino acids 2-500 of Q9H1K5 (SEQ ID NO:990), which also corresponds to amino acids 498-996 of R30650_PEA_{—}2_P5 (SEQ ID NO:992), wherein said first amino acid sequence and second amino acid sequence are contiguous and in a sequential order.

According to preferred embodiments of the present invention, there is provided an isolated polypeptide encoding for a head of R30650_PEA_{—}2_P5 (SEQ ID NO:992), comprising a polypeptide being at least 70%, optionally at least about 80%, preferably at least about 85%, more preferably at least about 90% and most preferably at least about 95% homologous to the sequence MDGVNLSTEVVYKKGQDYRFACYDRGRACRSYRVRFLCGKPVRPKLTVTIDTNVNSTILNLEDNVQSWKP GDTLVIASTDYSMYQAEEFQVLPCRSCAPNQVKVAGKPMYLHIGEEIDGVDMRAEVGLLSRNIIVMGEMED KCYPYRNHICNFFDFDTFGGHIKFALGFKAAHLEGTELKHMGQQLVGQYPIHFHLAGDVDERGGYDPPTYI RDLSIHHTFSRCVTVHGSNGLLIKDVVGYNSLGHCFFTEDGPEERNTFDHCLGLLVKSGTLLPSDRDSKMCK MITEDSYPGYIPKPRQDCNAVSTFWMANPNNNLINCAAAGSEETGFWFIFHHVPTGPSVGMYSPGYSEHIPL GKFYNNRAHSNYRAGMIIDNGVKTTEASAKDKRPFLSIISARYSPHQDADPLKPREPAIIRHFIAYKNQDHGA WLRGGDVWLDSCRFADNGIGLTLASGGTFPYDDGSKQEIKNSLFVGESGNVGTEMMDNRIWGPGGLDH of R30650_PEA_{—}2_P5 (SEQ ID NO:992).

According to preferred embodiments of the present invention, there is provided an isolated chimeric polypeptide encoding for R30650_PEA_{—}2_P8 (SEQ ID NO:993), comprising a first amino acid sequence being at least 70%, optionally at least 80%, preferably at least 85%, more preferably at least 90% and most preferably at least 95% homologous to a polypeptide having the sequence MGAAGRQDFLFKAMLTISWLTLTCFPGATSTVAAGCPDQSPELQPWNPGHDQDHHVHIGQGKTLLLTSSAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRHPWSFLTVKGNPSSSVEDHIE YHGHRGSAAARVFKLFQTEHGEYFNVSLSSEWVQDVEWTEWFDHDKVSQTKGGEKISDLWK (SEQ ID NO:1469) corresponding to amino acids 1-348 of R30650_PEA_{—}2_P8 (SEQ ID NO:993), a second amino acid sequence being at least 90% homologous to AHPGKICNRPIDIQATTMDGVNLSTEVVYKKGQDYRFACYDRGRACRSYRVRFLCGKPVRPKLTVTIDTNV NSTILNLEDNVQSWKPGDTLVIASTDYSMYQAEEFQVLPCRSCAPNQVKVAGKPMYLHIGEEIDGVDMRAE VGLLSRNIIVMGEMEDKCYPYRNHICNFFDFDTFGGHIKFALGFKAAHLEGTELKHMGQQLVGQYPIHFHL AGDVDERGGYDPPTYIRDLSIHHTFSRCVTVHGSNGLLIKDVVGYNSLGHCFFTEDGPEERNTFDHCLGLLV KSGTLLPSDRDSKMCKMITEDSYPGYIPKPRQDCNAVSTFWMANPNNNLINCAAAGSEETGFWFIFHHVPT GPSVGMYSPGYSEHIPLGKFYNNRAHSNYRAGMIIDNGVKTTEASAKDKRPFLSIISARYSPHQDADPLKPR EPAIIRHFIAYKNQDHGAWLRGGDVWLDSCRFADNGIGLTLASGGTFPYDDGSKQEIKNSLFVGESGNVGT EMMDNRIWGPGGLDHSGRTLPIGQNFPIRGIQLYDGPINIQNCTFRKFVALEGRHTSALAFRLNNAWQSCPH NNVTGIAFEDVPITSRVFFGEPGPWFNQLDMDGDKTSVFHDVDGSVSEYPGSYLTKNDNWLVRHPDCINVP DWRGAICSGCYAQMYIQAYKTSNLRMKIIKNDFPSHPLYLEGALTRSTHYQQYQPVVTLQKGYTIHWDQT APAELAIWLINFNKGDWIRVGLCYPRGTTFSILSDVHNRLLKQTSKTGVFVRTLQMDKVEQSYPGRSHYYW DEDSG corresponding to amino acids 1-788 of Q9ULM1 (SEQ ID NO:989), which also corresponds to amino acids 349-1136 of R30650_PEA_{—}2_P8 (SEQ ID NO:993), and a third amino acid sequence being at least 70%, optionally at least 80%, preferably at least 85%, more preferably at least 90% and most preferably at least 95% homologous to a polypeptide having the sequence KQRTISWR (SEQ ID NO:1470) corresponding to amino acids 1137-1144 of R30650_PEA_{—}2_P8 (SEQ ID NO:993), wherein said first amino acid sequence, second amino acid sequence and third amino acid sequence are contiguous and in a sequential order.

According to preferred embodiments of the present invention, there is provided an isolated polypeptide encoding for a head of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence MGAAGRQDFLFKAMLTISWLTLTCFPGATSTVAAGCPDQSPELQPWNPGHDQDHHVHIGQGKTLLLTSSAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRHPWSFLTVKGNPSSSVEDHIE YHGHRGSAAARVFKLFQTEHGEYFNVSLSSEWVQDVEWTEWFDHDKVSQTKGGEKISDLWK (SEQ ID NO:1469) of R30650_PEA_{—}2_P8 (SEQ ID NO:993).

According to preferred embodiments of the present invention, there is provided an isolated polypeptide encoding for a tail of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence KQRTISWR (SEQ ID NO:1470) in R30650_PEA_{—}2_P8 (SEQ ID NO:993).

According to preferred embodiments of the present invention, there is provided an isolated chimeric polypeptide encoding for R30650_PEA_{—}2_P8 (SEQ ID NO:993), comprising a first amino acid sequence being at least 90% homologous to MGAAGRQDFLFKAMLTISWLTLTCFPGATSTVAAGCPDQSPELQPWNPGHDQDHHVRHIGQGKTLLLTSSAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRIIPWSFLTVKGNPSSSVEDHIE YHGHRGSAAARVFKLFQTEHGEYFNVSLSSEWVQDVEWTEWFDHDKVSQTKGGEKISDLWKAHPGKICN RPIDIQATTMDGVNLSTEVVYKKGQDYRFACYDRGRACRSYRVRFLCGKPVRPKLTVTIDTNVNSTILNLE DNVQSWKPGDTLVIASTDYSMYQAEEFQVLPCRSCAPNQVKVAGKPMYLHIGEEIDGVDMRAEVGLLSRN IIVMGEMEDKCYPYRNHICNFFDFDTFGGHIKFALGFKAAHLEGTELKHMGQQLVGQYPIHFHLAGDVDER GGYDPPTYIRDLSIHHTFSRCVTVHGSNGLLIKDVVGYNSLGHCFFTEDGPEERNTFDHCLGLLVKSGTLLPS DRDSKMCKMITEDSYPGYIPKPRQDCNAVSTFWMANPNNNLINCAAAGSEETGFWFIFHHVPTGPSVGMYS PGYSEHIPLGKFYNNRAHSNYRAGMIIDNGVKTTEASAKDKRPFLSIISARYSPHQDADPLKPREPAIIRHFIA YKNQDHGAWLRGGDVWLDSCRFADNGIGLTLASGGTFPYDDGSKQEIKNSLFVGESGNVGTEMMDNRIW GPGGLDHSGRTLPIGQNFPIRGIQLYDGPINIQNCTFRKFVALEGRHTSALAFRLNNAWQSCPHNNVTGIAFE DVPITSRVFFGEPGPWFNQLDMDGDKTSVFHDVDGSVSEYPGSYLTKND corresponding to amino acids 1-977 of Q8WUJ3 (SEQ ID NO:987), which also corresponds to amino acids 1-977 of R30650_PEA_{—}2_P8 (SEQ ID NO:993), and a second amino acid sequence being at least 70%, optionally at least 80%, preferably at least 85%, more preferably at least 90% and most preferably at least 95% homologous to a polypeptide having the sequence NWLVRHPDCINVPDWRGAICSGCYAQMYIQAYKTSNLRMKIIKNDFPSHPLYLEGALTRSTHYQQYQPVVT LQKGYTIHWDQTAPAELAIWLINFNKGDWIRVGLCYPRGTTFSILSDVHNRLLKQTSKTGVFVRTLQMDKV EQSYPGRSHYYWDEDSGKQRTISWR corresponding to amino acids 978-1144 of R30650_PEA_{—}2_P8 (SEQ ID NO:993), wherein said first amino acid sequence and second amino acid sequence are contiguous and in a sequential order.

According to preferred embodiments of the present invention, there is provided an isolated polypeptide encoding for a tail of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence NWLVRHPDCINVPDWRGAICSGCYAQMYIQAYKTSNLRMKIIKNDFPSHPLYLEGALTRSTHYQQYQPVVT LQKGYTIHWDQTAPAELAIWLINFNKGDWIRVGLCYPRGTTFSILSDVHNRLLKQTSKTGVFVRTLQMDKV EQSYPGRSHYYWDEDSGKQRTISWR in R30650_PEA_{—}2_P8 (SEQ ID NO:993).

According to preferred embodiments of the present invention, there is provided an isolated chimeric polypeptide encoding for R30650_PEA_{—}2_P8 (SEQ ID NO:993), comprising a first amino acid sequence being at least 70%, optionally at least 80%, preferably at least 85%, more preferably at least 90% and most preferably at least 95% homologous to a polypeptide having the sequence MGAAGRQDFLFKAMLTISWLTLTCFPGATSTVAAGCPDQSPELQPWNPGHDQDHHVHIGQGKTLLLTSSAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRHPWSFLTVKGNPSSSVEDHIE YHGHRGSAAARVFKLFQTEHGEYFNVSLSSEWVQDVEWTEWFDHDKVSQTKGGEKISDLWKAHPGKICN RPIDIQATTMDGVNLSTEVVYKKGQDYRFACYDRGRACRSYRVRFLCGKPVRPKLTVTIDTNVNSTILNLE DNVQSWKPGDTLVIASTDYSMYQAEEFQVLPCRSCAPNQVKVAGKPMYLHIGEEIDGVDMRAEVGLLSRN IIVMGEMEDKCYPYRNHICNFFDFDTFGGHIKFALGFKAAHLEGTELKHMGQQLVGQYPIHFHLAGD corresponding to amino acids 1-564 of R30650_PEA_{—}2_P8 (SEQ ID NO:993), a second amino acid sequence being at least 90% homologous to VDERGGYDPPTYIRDLSIHHTFSRCVTVHGSNGLLIKDVVGYNSLGHCFFTEDGPEERNTFDHCLGLLVKSG TLLPSDRDSKMCKMITEDSYPGYIPKPRQDCNAVSTFWMANPNNNLINCAAAGSEETGFWFIFHHVPTGPSV GMYSPGYSEHIPLGKFYNNRAHSNYRAGMIIDNGVKTTEASAKDKRPFLSIISARYSPHQDADPLKPREPAI RHFIAYKNQDHGAWLRGGDVWLDSCRFADNGIGLTLASGGTFPYDDGSKQEIKNSLFVGESGNVGTEMMD NRIWGPGGLDHSGRTLPIGQNFPIRGIQLYDGPINIQNCTFRKFVALEGRHTSALAFRLNNAWQSCPHNNVT GIAFEDVPITSRVFFGEPGPWFNQLDMDGDKTSVFHDVDGSVSEYPGSYLTKNDNWLVRHPDCINVPDWRG AICSGCYAQMYIQAYKTSNLRMKIIKNDFPSIHPLYLEGALTRSTHYQQYQPVVTLQKGYTIHWDQTAPAEL AIWLINFNKGDWIRVGLCYPRGTTFSILSDVHNRLLKQTSKTGVFVRTLQMDKVEQSYPGRSHYYWDEDSG corresponding to amino acids 8-579 of Q9NPN9 (SEQ ID NO:988), which also corresponds to amino acids 565-1136 of R30650_PEA_{—}2_P8 (SEQ ID NO:993), and a third amino acid sequence being at least 70%, optionally at least 80%, preferably at least 85%, more preferably at least 90% and most preferably at least 95% homologous to a polypeptide having the sequence KQRTISWR (SEQ ID NO:1470) corresponding to amino acids 1137-1144 of R30650_PEA_{—}2_P8 (SEQ ID NO:993), wherein said first amino acid sequence, second amino acid sequence and third amino acid sequence are contiguous and in a sequential order.

According to preferred embodiments of the present invention, there is provided an isolated polypeptide encoding for a head of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence MGAAGRQDFLFKAMLTISWLTLTCFPGATSTVAAGCPDQSPELQPWNPGHDQDHHVHIGQGKTLLLTSSAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRHPWSFLTVKGNPSSSVEDHIE YHGHRGSAAARVFKLFQTEHGEYFNVSLSSEWVQDVEWTEWFDHDKVSQTKGGEKISDLWKAHPGKICN RPIDIQATTMDGVNLSTEVVYKKGQDYRFACYDRGRACRSYRVRFLCGKPVRPKLTVTIDTNVNSTILNLE DNVQSWKPGDTLVIASTDYSMYQAEEFQVLPCRSCAPNQVKVAGKPMYLHIGEEIDGVDMRAEVGLLSRN IIVMGEMEDKCYPYRNHICNFFDFDTFGGHIKFALGFKAAHLEGTELKHMGQQLVGQYPIHFHLAGD of R30650_PEA_{—}2_P8 (SEQ ID NO:993).

According to preferred embodiments of the present invention, there is provided an isolated polypeptide encoding for a tail of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence KQRTISWR (SEQ ID NO:1470) in R30650_PEA_{—}2_P8 (SEQ ID NO:993).

According to preferred embodiments of the present invention, there is provided an isolated chimeric polypeptide encoding for R30650_PEA_{—}2_P8 (SEQ ID NO:993), comprising a first amino acid sequence being at least 70%, optionally at least 80%, preferably at least 85%, more preferably at least 90% and most preferably at least 95% homologous to a polypeptide having the sequence MGAAGRQDFLFKAMLTISWLTLTCFPGATSTVAAGCPDQSPELQPWNPGHDQDHHVHIGQGKTLLLTSSAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRHPWSFLTVKGNPSSSVEDHIE YHGHRGSAAARVFKLFQTEHGEYFNVSLSSEWVQDVEWTEWFDHDKVSQTKGGEKISDLWKAHPGKICN RPIDIQATTMDGVNLSTEVVYKKGQDYRFACYDRGRACRSYRVRFLCGKPVRPKLTVTIDTNVNSTILNLE DNVQSWKPGDTLVIASTDYSMYQAEEFQVLPCRSCAPNQVKVAGKPMYLHIGEEIDGVDMRAEVGLLSRN IIVMGEMEDKCYPYRNHICNFFDFDTFGGHIKFALGFKAAHLEGTELKHMGQQLVGQYPIHFHLAGDVDER GGYDPPTYIRDLSIHHTFSRCVTVHGSNGLLIKDVVGYNSLGHCFFTEDGPEERNTFDHCLGLLVKSGTLLPS DRDSKMCKMITEDSYPGYIPKPRQDCNAVSTFWMANPNNNLINCAAAGSEETGFWFIFHHVPTGPSVGMYS PGYSEHIPLGKFYNNRAHSNYRAGMIIDNGVKTTEASAKDKRPFLSIISARYSPHQDADPLKPREPAIIRHFIA YKNQDHGAWLRGGDVWLDSCRFADNGIGLTLASGGTFPYDDGSKQEIKNSLFVGESGNVGTEMMDNRIW GPGGLDH corresponding to amino acids 1-862 of R30650_PEA_{—}2_P8 (SEQ ID NO:993), a second amino acid sequence being at least 90% homologous to SGRTLPIGQNFPIRGIQLYDGPINIQNCTFRKFVALEGRHTSALAFRLNNAWQSCPHNNVTGIAFEDVPITSRV FFGEPGPWFNQLDMDGDKTSVFHDVDGSVSEYPGSYLTKNDNWLVRHPDCINVPDWRGAICSGCYAQMYI QAYKTSNLRMKIIKNDFPSHPLYLEGALTRSTHYQQYQPVVTLQKGYTIHWDQTAPAELAIWLINFNKGDW IRVGLCYPRGTTFSILSDVHNRLLKQTSKTGVFVRTLQMDKVEQSYPGRSHYYWDEDSG corresponding to amino acids 2-275 of Q9H1K5 (SEQ ID NO:990), which also corresponds to amino acids 863-1136 of R30650_PEA_{—}2_P8 (SEQ ID NO:993), and a third amino acid sequence being at least 70%, optionally at least 80%, preferably at least 85%, more preferably at least 90% and most preferably at least 95% homologous to a polypeptide having the sequence KQRTISWR (SEQ ID NO:1470) corresponding to amino acids 1137-1144 of R30650_PEA_{—}2_P8 (SEQ ID NO:993), wherein said first amino acid sequence, second amino acid sequence and third amino acid sequence are contiguous and in a sequential order.

According to preferred embodiments of the present invention, there is provided an isolated polypeptide encoding for a head of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence MGAAGRQDFLFKAMLTISWLTLTCFPGATSTVAAGCPDQSPELQPWNPGHDQDHHVHIGQGKTLLLTSSAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRHPWSFLTVKGNPSSSVEDHIE YHGHRGSAAARVFKLFQTEHGEYFNVSLSSEWVQDVEWTEWFDHDKVSQTKGGEKISDLWKAHPGKICN RPIDIQATTMDGVNLSTEVVYKKGQDYRFACYDRGRACRSYRVRFLCGKPVRPKLTVTIDTNVNSTILNLE DNVQSWKPGDTLVIASTDYSMYQAEEFQVLPCRSCAPNQVKVAGKPMYLHIGEEIDGVDMRAEVGLLSRN IIVMGEMEDKCYPYRNHICNFFDFDTFGGHIKFALGFKAAHLEGTELKHMGQQLVGQYPIHFHLAGDVDER GGYDPPTYIRDLSIHHTFSRCVTVHGSNGLLIKDVVGYNSLGHCFFTEDGPEERNTFDHCLGLLVKSGTLLPS DRDSKMCKMITEDSYPGYIPKPRQDCNAVSTFWMANPNNNLINCAAAGSEETGFWFIFHHVPTGPSVGMYS PGYSEHIPLGKFYNNRAHSNYRAGMIIDNGVKTTEASAKDKRPFLSIISARYSPHQDADPLKPREPAIIRHFIA YKNQDHGAWLRGGDVWLDSCRFADNGIGLTLASGGTFPYDDGSKQEIKNSLFVGESGNVGTEMMDNRIW GPGGLDH of R30650_PEA_{—}2_P8 (SEQ ID NO:993).

According to preferred embodiments of the present invention, there is provided an isolated polypeptide encoding for a tail of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence KQRTISWR (SEQ ID NO:1470) in R30650_PEA_{—}2_P8 (SEQ ID NO:993).

According to preferred embodiments of the present invention, there is provided an isolated chimeric polypeptide encoding for R30650_PEA_{—}2_P15 (SEQ ID NO:996), comprising a first amino acid sequence being at least 70%, optionally at least 80%, preferably at least 85%, more preferably at least 90% and most preferably at least 95% homologous to a polypeptide having the sequence MGAAGRQDFLFKAMLTISWLTLTCFPGATSTVAAGCPDQSPELQPWNPGHDQDHHVHIGQGKTLLLTS SAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRHPWSFLTVKGNPSSSVEDHIE YHGHRGSAAARVFKLFQTEHGEYFNVSLSSEWVQDVEWTEWFDHDKVSQTKGGEKISDLWK (SEQ ID NO:1469) corresponding to amino acids 1-348 of R30650_PEA_{—}2_P15 (SEQ ID NO:996), and a second amino acid sequence being at least 90% homologous to AHPGKICNRPIDIQATTMDGVNLSTEVVYKKGQDYRFACYDRGRACRSYRVRFLCGKPVRPKLTVTIDTNV NSTILNLEDNVQSWKPGDTLVIASTDYSMYQAEEFQVLPCRSCAPNQVKVAGKPMYLHIGEEIDGVDMRAE VGLLSRNIIVMGEMEDKCYPYRNHICNFFDFDTFGGHIKFALGFKAAHLEGTELKHMGQQLVGQYPIHFHL AGDVDERGGYDPPTYIRDLSIHHTFSRCVTVHGSNGLLIKDVVGYNSLGHCFFTEDGPEERNTFDHCLGLLV KSGTLLPSDRDSKMCKMITEDSYPGYIPKPRQDCNAVSTFWMANPNNNLINCAAAGSEETGFWFIFHHVPT GPSVGMYSPGYSEHIPLGKFYNNRAHSNYRAGMIIDNGVKTTEASAKDKRPFLSIISARYSPHQDADPLKPR EPAIIRHFIAYKNQDHGAWLRGGDVWLDSCRFADNGIGLTLASGGTFPYDDGSKQEIKNSLFVGESGNVGT EMMDNRIWGPGGLDHSGRTLPIGQNFPIRGIQLYDGPINIQNCTFRKFVALEGRHTSALAFRLNNAWQSCPH NNVTGIAFEDVPITSRVFFGEPGPWFNQLDMDGDKTSVFHDVDGSVSEYPGSYLTKNDNWLVRHPDCINVP DWRGAICSGCYAQMYIQAYKTSNLRMKIIKNDFPSHPLYLEGALTRSTHYQQYQPVVTLQKGYTIHWDQT APAELAIWLINFNKGDWIRVGLCYPRGTTFSILSDVHNRLLKQTSKTGVFVRTLQMDKVEQSYPGRSHYYW DEDSG corresponding to amino acids 1-788 of Q9ULM1 (SEQ ID NO:989), which also corresponds to amino acids 349-1136 of R30650_PEA_{—}2_P15 (SEQ ID NO:996), wherein said first amino acid sequence and second amino acid sequence are contiguous and in a sequential order.

According to preferred embodiments of the present invention, there is provided an isolated polypeptide encoding for a head of R30650_PEA_{—}2_P15 (SEQ ID NO:996), comprising a polypeptide being at least 70%, optionally at least about 80%, preferably at least about 85%, more preferably at least about 90% and most preferably at least about 95% homologous to the sequence MGAAGRQDFLFKAMLTISWLTLTCFPGATSTVAAGCPDQSPELQPWNPGHDQDHHVHIGQGKTLLLTS SAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRPWSFLTVKGNPSSSVEDHIE YHGHRGSAAARVFKLFQTEHGEYFNVSLSSEWVQDVEWTEWFDHDKVSQTKGGEKISDLWK (SEQ ID NO:1469) of R30650_PEA_{—}2_P15 (SEQ ID NO:996).

According to preferred embodiments of the present invention, there is provided an isolated chimeric polypeptide encoding for R30650_PEA_{—}2_P15 (SEQ ID NO:996), comprising a first amino acid sequence being at least 90% homologous to MGAAGRQDFLFKAMLTISWLTLTCFPGATSTVAAGCPDQSPELQPWNPGHDQDHHVHIGQGKTLLLTSSAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRHPWSFLTVKGNPSSSVEDHIE YHGHRGSAAARVFKLFQTEHGEYFNVSLSSEWVQDVEWTEWFDHDKVSQTKGGEKISDLWKAHPGKICN RPIDIQATTMDGVNLSTEVVYKKGQDYRFACYDRGPACRSYRVRFLCGKPVRPKLTVTIDTNVNSTILNLE DNVQSWKPGDTLVIASTDYSMYQAEEFQVLPCRSCAPNQVKVAGKPMYLHIGEEIDGVDMRAEVGLLSRN IIVMGEMEDKCYPYRNHICNFFDFDTFGGHIKFALGFKAAHLEGTELKHMGQQLVGQYPIHFHLAGDVDER GGYDPPTYIRDLSIHHTFSRCVTVHGSNGLLIKDVVGYNSLGHCFFTEDGPEERNTFDHCLGLLVKSGTLLPS DRDSKMCKMITEDSYPGYIPKPRQDCNAVSTFWMANPNNNLINCAAAGSEETGFWFIFHHVPTGPSVGMYS PGYSEHIPLGKFYNNRAHSNYRAGMIIDNGVKTTEASAKDKRPFLSIISARYSPHQDADPLKPREPAIIRHFIA YKNQDHGAWLRGGDVWLDSCRFADNGIGLTLASGGTFPYDDGSKQEIKNSLFVGESGNVGTEMMDNRIW GPGGLDHSGRTLPIGQNFPIRGIQLYDGPINIQNCTFRKFVALEGRHTSALAFRLNNAWQSCPHNNVTGIAFE DVPITSRVFFGEPGPWFNQLDMDGDKTSVFHDVDGSVSEYPGSYLTKND corresponding to amino acids 1-977 of Q8WUJ3 (SEQ ID NO:987), which also corresponds to amino acids 1-977 of R30650_PEA_{—}2_P15 (SEQ ID NO:996), and a second amino acid sequence being at least 70%, optionally at least 80%, preferably at least 85%, more preferably at least 90% and most preferably at least 95% homologous to a polypeptide having the sequence NWLVRHPDCINVPDWRGAICSGCYAQMYIQAYKTSNLRMKIIKNDFPSHPLYLEGALTRSTHYQQYQPVVT LQKGYTIHWDQTAPAELAIWLINFNKGDWIRVGLCYPRGTTFSILSDVHNRLLKQTSKTGVFVRTLQMDKV EQSYPGRSHYYWDEDSG (SEQ ID NO:1472) corresponding to amino acids 978-1136 of R30650_PEA_{—}2_P15 (SEQ ID NO:996), wherein said first amino acid sequence and second amino acid sequence are contiguous and in a sequential order.

According to preferred embodiments of the present invention, there is provided an isolated polypeptide encoding for a tail of R30650_PEA_{—}2_P15 (SEQ ID NO:996), comprising a polypeptide being at least 70%, optionally at least about 80%, preferably at least about 85%, more preferably at least about 90% and most preferably at least about 95% homologous to the sequence NWLVRHPDCINVPDWRGAICSGCYAQMYIQAYKTSNLRMKIIKNDFPSHPLYLEGALTRSTHYQQYQPVVT LQKGYTIHWDQTAPAELAIWLINFNKGDWIRVGLCYPRGTTFSILSDVHNRLLKQTSKTGVFVRTLQMDKV EQSYPGRSHYYWDEDSG (SEQ ID NO:1472) in R30650_PEA_{—}2_P15 (SEQ ID NO:996).

According to preferred embodiments of the present invention, there is provided an isolated chimeric polypeptide encoding for R30650_PEA_{—}2_P15 (SEQ ID NO:996), comprising a first amino acid sequence being at least 70%, optionally at least 80%, preferably at least 85%, more preferably at least 90% and most preferably at least 95% homologous to a polypeptide having the sequence MGAAGRQDFLFKAMLTISWLTLTCFPGATSTVAAGCPDQSPELQPWNPGHDQDHHVHIGQGKTLLLTSSAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRHPWSFLTVKGNPSSSVEDHIE YHGHRGSAAARVFKLFQTEHGEYFNVSLSSEWVQDVEWTEWFDHDKVSQTKGGEKISDLWKAHPGKICN RPIDIQATTMDGVNLSTEVVYKKGQDYRFACYDRGRACRSYRVRFLCGKPVRPKLTVTIDTNVNSTILNLE DNVQSWKPGDTLVIASTDYSMYQAEEFQVLPCRSCAPNQVKVAGKPMYLHIGEEIDGVDMRAEVGLLSRN IIVMGEMEDKCYPYRNHICNFFDFDTFGGHIKFALGFKAAHLEGTELKHMGQQLVGQYPIHFHLAGD corresponding to amino acids 1-564 of R30650_PEA_{—}2_P15 (SEQ ID NO:996), and a second amino acid sequence being at least 90% homologous to VDERGGYDPPTYIRDLSIHHTFSRCVTVHGSNGLLIKDVVGYNSLGHCFFTEDGPEERNTFDHCLGLLVKSG TLLPSDRDSKMCKMITEDSYPGYIPKPRQDCNAVSTFWMANPNNNLINCAAAGSEETGFWFIFHHVPTGPSV GMYSPGYSEHIPLGKFYNNRAHSNYRAGMIIDNGVKTTEASAKDKRPFLSIISARYSPHQDADPLKPREPAII RHFIAYKNQDHGAWLRGGDVWLDSCRFADNGIGLTLASGGTFPYDDGSKQEIKNSLFVGESGNVGTEMMD NRIWGPGGLDHSGRTLPIGQNFPIRGIQLYDGPINIQNCTFRKFVALEGRHTSALAFRLNNAWQSCPHNNVT GIAFEDVPITSRVFFGEPGPWFNQLDMDGDKTSVFHDVDGSVSEYPGSYLTKNDNWLVRHPDCINVPDWRG AICSGCYAQMYIQAYKTSNLRMKIIKNDFPSHPLYLEGALTRSTHYQQYQPVVTLQKGYTIHWDQTAPAEL AIWLINFNKGDWIRVGLCYPRGTTFSILSDVHNRLLKQTSKTGVFVRTLQMDKVEQSYPGRSHYYWDEDSG corresponding to amino acids 8-579 of Q9NPN9 (SEQ ID NO:988), which also corresponds to amino acids 565-1136 of R30650_PEA_{—}2_P15 (SEQ ID NO:996), wherein said first amino acid sequence and second amino acid sequence are contiguous and in a sequential order.

According to preferred embodiments of the present invention, there is provided an isolated polypeptide encoding for a head of R30650_PEA_{—}2_P15 (SEQ ID NO:996), comprising a polypeptide being at least 70%, optionally at least about 80%, preferably at least about 85%, more preferably at least about 90% and most preferably at least about 95% homologous to the sequence MGAAGRQDFLFKAMLTISWLTLTCFPGATSTVAAGCPDQSPELQPWNPGHDQDHHVHIGQGKTLLLTSSAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRHPWSFLTVKGNPSSSVEDHIE YHGHRGSAAARVFKLFQTEHGEYFNVSLSSEWVQDVEWTEWFDHDKVSQTKGGEKISDLWKAHPGKICN RPIDIQATTMDGVNLSTEVVYKKGQDYRFACYDRGRACRSYRVRFLCGKPVRPKLTVTIDTNVNSTILNLE DNVQSWKPGDTLVIASTDYSMYQAEEFQVLPCRSCAPNQVKVAGKPMYLHIGEEIDGVDMRAEVGLLSRN IIVMGEMEDKCYPYRNHICNFFDFDTFGGHIKFALGFKAAHLEGTELKHMGQQLVGQYPIHFHLAGD of R30650_PEA_{—}2_P15 (SEQ ID NO:996).

According to preferred embodiments of the present invention, there is provided an isolated chimeric polypeptide encoding for R30650_PEA_{—}2_P15 (SEQ ID NO:996), comprising a first amino acid sequence being at least 70%, optionally at least 80%, preferably at least 85%, more preferably at least 90% and most preferably at least 95% homologous to a polypeptide having the sequence MGAAGRQDFLFKAMLTISWLTLTCFPGATSTVAAGCPDQSPELQPWNPGHDQDHHVHIGQGKTLLLTS SAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRHPWSFLTVKGNPSSSVEDHIE YHGHRGSAAARVFKLFQTEHGEYFNVSLSSEWVQDVEWTEWFDHDKVSQTKGGEKISDLWKAHPGKICN RPIDIQATTMDGVNLSTEVVYKKGQDYRFACYDRGRACRSYRVRFLCGKPVRPKLTVTIDTNVNSTILNLE DNVQSWKPGDTLVIASTDYSMYQAEEFQVLPCRSCAPNQVKVAGKPMYLHIGEEIDGVDMRAEVGLLSRN IIVMGEMEDKCYPYRNHICNFFDFDTFGGHIKFALGFKAAHLEGTELKHMGQQLVGQYPIHFHLAGDVDER GGYDPPTYIRDLSIHHTFSRCVTVHGSNGLLIKDVVGYNSLGHCFFTEDGPEERNTFDHCLGLLVKSGTLLPS DRDSKMCKMITEDSYPGYIPKPRQDCNAVSTFWMANPNNNLINCAAAGSEETGFWFIFHHVPTGPSVGMYS PGYSEHIPLGKFYNNRAHSNYRAGMIIDNGVKTTEASAKDKRPFLSIISARYSPHQDADPLKPREPAIIRHFIA YKNQDHGAWLRGGDVWLDSCRFADNGIGLTLASGGTFPYDDGSKQEIKNSLFVGESGNVGTEMMDNRIW GPGGLDH corresponding to amino acids 1-862 of R30650_PEA_{—}2_P15 (SEQ ID NO:996), and a second amino acid sequence being at least 90% homologous to SGRTLPIGQNFPIRGIQLYDGPINIQNCTFRKFVALEGRHTSALAFRLNNAWQSCPHNNVTGIAFEDVPITSRV FFGEPGPWFNQLDMDGDKTSVFHDVDGSVSEYPGSYLTKNDNWLVRHPDCINVPDWRGAICSGCYAQMYI QAYKTSNLRMKIIKNDFPSHPLYLEGALTRSTHYQQYQPVVTLQKGYTIHWDQTAPAELAIWLINFNKGDW IRVGLCYPRGTTFSILSDVHNRLLKQTSKTGVFVRTLQMDKVEQSYPGRSHYYWDEDSG corresponding to amino acids 2-275 of Q9H1K5 (SEQ ID NO:990), which also corresponds to amino acids 863-1136 of R30650_PEA_{—}2_P15 (SEQ ID NO:996), wherein said first amino acid sequence and second amino acid sequence are contiguous and in a sequential order.

According to preferred embodiments of the present invention, there is provided an isolated polypeptide encoding for a head of R30650_PEA_{—}2_P15 (SEQ ID NO:996), comprising a polypeptide being at least 70%, optionally at least about 80%, preferably at least about 85%, more preferably at least about 90% and most preferably at least about 95% homologous to the sequence MGAAGRQDFLFKAMLTISWLTLTCFPGATSTVAAGCPDQSPELQPWNPGHDQDHHVHIGQGKTLLLTSSAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRHPWSFLTVKGNPSSSVEDHIE YHGHRGSAAARVFKLFQTEHGEYFNVSLSSEWVQDVEWTEWFDHDKVSQTKGGEKISDLWKAHPGKICN RPIDIQATTMDGVNLSTEVVYKKGQDYRFACYDRGRACRSYRVRFLCGKPVRPKLTVTIDTNVNSTILNLE DNVQSWKPGDTLVIASTDYSMYQAEEFQVLPCRSCAPNQVKVAGKPMYLHIGEEIDGVDMRAEVGLLSRN IIVMGEMEDKCYPYRNHICNFFDFDTFGGHIKFALGFKAAHLEGTELKHMGQQLVGQYPIHFHLAGDVDER GGYDPPTYIRDLSIHHTFSRCVTVHGSNGLLIKDVVGYNSLGHCFFTEDGPEERNTFDHCLGLLVKSGTLLPS DRDSKMCKMITEDSYPGYIPKPRQDCNAVSTFWMANPNNNLINCAAAGSEETGFWFIFHHVPTGPSVGMYS PGYSEHIPLGKFYNNRAHSNYRAGMIIDNGVKTTEASAKDKRPFLSIISARYSPHQDADPLKPREPAIIRHFIA YKNQDHGAWLRGGDVWLDSCRFADNGIGLTLASGGTFPYDDGSKQEIKNSLFVGESGNVGTEMMDNRIW GPGGLDH of R30650_PEA_{—}2_P15 (SEQ ID NO:996).

According to preferred embodiments of the present invention, there is provided an isolated chimeric polypeptide encoding for R30650_PEA_{—}2_P17 (SEQ ID NO:997), comprising a first amino acid sequence being at least 90% homologous to MGAAGRQDFLFKAMLTISWLTLTCFPGATSTVAAGCPDQSPELQPWNPGHDQDHHVHIGQGKTLLLTSSAT VYSIHISEGGKLVIKDHDEPIVLRTRHILIDNGGELHAGSALCPFQGNFTIILYGRADEGIQPDPYYGLKYIGV GKGGALELHGQKKLSWTFLNKTLHPGGMAEGGYFFERSWGHRGVIVHVIDPKSGTVIHSDRFDTYRSKKES ERLVQYLNAVPDGRILSVAVNDEGSRNLDDMARKAMTKLGSKHFLHLGFRHPWSFLTVKGNPSSSVEDHIE YHGHRGSAAARVFKLFQTEHGEYFNVSLSSEWVQ corresponding to amino acids 1-321 of Q8WUJ3 (SEQ ID NO:987), which also corresponds to amino acids 1-321 of R30650_PEA_{—}2_P17 (SEQ ID NO:997), and a second amino acid sequence being at least 70%, optionally at least 80%, preferably at least 85%, more preferably at least 90% and most preferably at least 95% homologous to a polypeptide having the sequence GEEFQTIW (SEQ ID NO:1473) corresponding to amino acids 322-329 of R30650_PEA_{—}2_P17 (SEQ ID NO:997), wherein said first amino acid sequence and second amino acid sequence are contiguous and in a sequential order.

According to preferred embodiments of the present invention, there is provided an isolated polypeptide encoding for a tail of R30650_PEA_{—}2_P17 (SEQ ID NO:997), comprising a polypeptide being at least 70%, optionally at least about 80%, preferably at least about 85%, more preferably at least about 90% and most preferably at least about 95% homologous to the sequence GEEFQTIW (SEQ ID NO:1473) in R30650_PEA_{—}2_P17 (SEQ ID NO:997).

According to preferred embodiments of the present invention, there is provided an isolated chimeric polypeptide encoding for M78035_P4 (SEQ ID NO:924), comprising a first amino acid sequence being at least 90% homologous to MPGLMRMRERYSASKPLKGARIAGCLHMTVETAVLIETLVTLGAEVQWSSCNIFSTQDHAAAAIAKAGIPV YAWKGETDEEYLWCIEQTLYFKDGPLNMILDDGGDLTNLIHTKYPQLLPGIRGISEETTTGVHNLYKMMAN GILKVPAINVNDSVTKSKFDNLYGCRESLIDGIKRATDVMIAGKVAVVAGYGDVGKGCAQALRGFGARVII TEIDPINALQAAMEGYEVTTMDEACQEGNIFVTTTGCIDIILGRHFEQMKDDAIVCNIGHFDVEIDVKWLNE NAVEKVNIKPQVDRYRLKNGRRIILLAEGRLVNLGCAMGHPSFVMSNSFTNQVMAQIELWTHPDKYPVGV HFLPKKLDEAVAEAHLGKLNVKLTKLTEKQAQYLGMSCDGPFKPDHYRY corresponding to amino acids 29-432 of SAHH_HUMAN (SEQ ID NO:922), which also corresponds to amino acids 1-404 of M78035_P4 (SEQ ID NO:924).

According to preferred embodiments of the present invention, there is provided an isolated chimeric polypeptide encoding for M78035_P6 (SEQ ID NO:925), comprising a first amino acid sequence being at least 90% homologous to MILDDGGDLTNLIHTKYPQLLPGIRGISEETTTGVHNLYKMMANGILKVPAINVNDSVTKSKFDNLYGCRES LIDGIKRATDVMIAGKVAVVAGYGDVGKGCAQALRGFGARVIITEIDPINALQAAMEGYEVTTMDEACQE GNIFVTTTGCIDIILGRHFEQMKDDAIVCNIGHFDVEIDVKWLNENAVEKVNIKPQVDRYRLKNGRRIILLAE GRLVNLGCAMGHPSFVMSNSFTNQVMAQIELWTHPDKYPVGVHFLPKKLDEAVAEAHLGKLNVKLTKLT EKQAQYLGMSCDGPFKPDHYRY corresponding to amino acids 127-432 of SAHH_HUMAN (SEQ ID NO:922), which also corresponds to amino acids 1-306 of M78035_P6 (SEQ ID NO:925).

According to preferred embodiments of the present invention, there is provided an isolated chimeric polypeptide encoding for M78035_P8 (SEQ ID NO:926), comprising a first amino acid sequence being at least 70%, optionally at least 80%, preferably at least 85%, more preferably at least 90% and most preferably at least 95% homologous to a polypeptide having the sequence MSDKLPYKV (SEQ ID NO:1474) corresponding to amino acids 1-9 of M78035_P8 (SEQ ID NO:926), and a second amino acid sequence being at least 90% homologous to VYAWKGETDEEYLWCIEQTLYFKDGPLNMILDDGGDLTNLIHTKYPQLLPGIRGISEETTTGVHNLYKMMA NGILKVPAINVNDSVTKSKFDNLYGCRESLIDGIKRATDVMIAGKVAVVAGYGDVGKGCAQALRGFGARVI ITEIDPINALQAAMEGYEVTTMDEACQEGNIFVTTTGCIDIILGRHFEQMKDDAIVCNIGHFDVEIDVKWLNE NAVEKVNIKPQVDRYRLKNGRRIILLAEGRLVNLGCAMGHPSFVMSNSFTNQVMAQIELWTHPDKYPVGV HFLPKKLDEAVAEAHLGKLNVKLTKLTEKQAQYLGMSCDGPFKPDHYRY corresponding to amino acids 99-432 of SAHH_HUMAN (SEQ ID NO:922), which also corresponds to amino acids 10-343 of M78035_P8 (SEQ ID NO:926), wherein said first amino acid sequence and second amino acid sequence are contiguous and in a sequential order.

According to preferred embodiments of the present invention, there is provided an isolated polypeptide encoding for a head of M78035_P8 (SEQ ID NO:926), comprising a polypeptide being at least 70%, optionally at least about 80%, preferably at least about 85%, more preferably at least about 90% and most preferably at least about 95% homologous to the sequence MSDKLPYKV (SEQ ID NO:1474) of M78035_P8 (SEQ ID NO:926).

According to preferred embodiments of the present invention, there is provided an isolated chimeric polypeptide encoding for HUMCEA_PEA_{—}1_P4 (SEQ ID NO:864), comprising a first amino acid sequence being at least 90% homologous to MESPSAPPHRWCIPWQRLLLTASLLTFWNPPTTAKLTIESTPFNVAEGKEVLLLVHNLPQHLFGYSWYKGER VDGNRQIIGYVIGTQQATPGPAYSGREIIYPNASLLIQNIIQNDTGFYTLHVIKSDLVNEEATGQFRVYPELPK PSISSNNSKPVEDKDAVAFTCEPETQDATYLWWVNNQSLPVSPRLQLSNGNRTLTLFNVTRNDTASYKCET QNPVSARRSDSVILNVL corresponding to amino acids 1-234 of CEA5_HUMAN (SEQ ID NO:863), which also corresponds to amino acids 1-234 of HUMCEA_PEA_{—}1_P4 (SEQ ID NO:864), and a second amino acid sequence being at least 70%, optionally at least 80%, preferably at least 85%, more preferably at least 90% and most preferably at least 95% homologous to a polypeptide having the sequence CEYICSSLAQAASPNPQGQRQDFSVPLRFKYTDPQPWTSRLSVTFCPRKTWADQVLTKNRRGGAASVLGGS GSTPYDGRNR (SEQ ID NO:1475) corresponding to amino acids 235-315 of HUMCEA_PEA_{—}1_P4 (SEQ ID NO:864), wherein said first amino acid sequence and second amino acid sequence are contiguous and in a sequential order.

According to preferred embodiments of the present invention, there is provided an isolated polypeptide encoding for a tail of HUMCEA_PEA_{—}1_P4 (SEQ ID NO:864), comprising a polypeptide being at least 70%, optionally at least about 80%, preferably at least about 85%, more preferably at least about 90% and most preferably at least about 95% homologous to the sequence CEYICSSLAQAASPNPQGQRQDFSVPLRFKYTDPQPWTSRLSVTFCPRKTWADQVLTKNRRGGAASVLGGS GSTPYDGRNR (SEQ ID NO:1475) in HUMCEA_PEA_{—}1_P4 (SEQ ID NO:864).

According to preferred embodiments of the present invention, there is provided an isolated chimeric polypeptide encoding for HUMCEA_PEA_{—}1_P5 (SEQ ID NO:865), comprising a first amino acid sequence being at least 90% homologous to MESPSAPPHRWCIPWQRLLLTASLLTFWNPPTTAKLTIESTPFNVAEGKEVLLLVHNLPQHLFGYSWYKGER VDGNRQIIGYVIGTQQATPGPAYSGREIIYPNASLLIQNIIQNDTGFYTLHVIKSDLVNEEATGQFRVYPELPK PSISSNNSKPVEDKDAVAFTCEPETQDATYLWWVNNQSLPVSPRLQLSNGNRTLTLFNVTRNDTASYKCET QNPVSARRSDSVILNVLYGPDAPTISPLNTSYRSGENLNLSCHAASNPPAQYSWFVNGTFQQSTQELFIPNIT VNNSGSYTCQAHNSDTGLNRTTVTTITVYAEPPKPFITSNNSNPVEDEDAVALTCEPEIQNTTYLWWVNNQS LPVSPRLQLSNDNRTLTLLSVTRNDVGPYECGIQNELSVDHSDPVILNVLYGPDDPTISPSYTYYRPGVNLSL SCHAASNPPAQYSWLIDGNIQQHTQELFISNITEKNSGLYTCQANNSASGHSRTTVKTITVSAELPKPSISSNN SKPVEDKDAVAFTCEPEAQNTTYLWWVNGQSLPVSPRLQLSNGNRTLTLFNVTRNDARAYVCGIQNSVSA NRSDPVTLDVLYGPDTPIISPPDSSYLSGANLNLSCHSASNPSPQYSWRINGIPQQHTQVLFIAKITPNNNGTY ACFVSNLATGRNNSIVKSITVS corresponding to amino acids 1-675 of CEA5_HUMAN (SEQ ID NO:863), which also corresponds to amino acids 1-675 of HUMCEA_PEA_{—}1_P5 (SEQ ID NO:865), and a second amino acid sequence being at least 70%, optionally at least 80%, preferably at least 85%, more preferably at least 90% and most preferably at least 95% homologous to a polypeptide having the sequence GKWLPGASASYSGVESIWFSPKSQEDIFFPSLCSMGTRKSQILS (SEQ ID NO:1476) corresponding to amino acids 676-719 of HUMCEA_PEA_{—}1_P5 (SEQ ID NO:865), wherein said first amino acid sequence and second amino acid sequence are contiguous and in a sequential order.

According to preferred embodiments of the present invention, there is provided an isolated polypeptide encoding for a tail of HUMCEA_PEA_{—}1_P5 (SEQ ID NO:865), comprising a polypeptide being at least 70%, optionally at least about 80%, preferably at least about 85%, more preferably at least about 90% and most preferably at least about 95% homologous to the sequence GKWLPGASASYSGVESIWFSPKSQEDIFFPSLCSMGTRKSQILS (SEQ ID NO:1476) in HUMCEA_PEA_{—}1_P5 (SEQ ID NO:865).

According to preferred embodiments of the present invention, there is provided an isolated chimeric polypeptide encoding for HUMCEA_PEA_{—}1_P7 (SEQ ID NO:866), comprising a first amino acid sequence being at least 90% homologous to MESPSAPPHRWCIPWQRLLLTASLLTFWNPPTTAKLTIESTPFNVAEGKEVLLLVHNLPQHLFGYSWYKGER VDGNRQIIGYVIGTQQATPGPAYSGREIIYPNASLLIQNIIQNDTGFYTLHVIKSDLVNEEATGQFRVYPELPK PSISSNNSKPVEDKDAVAFTCEPETQDATYLWWVNNQSLPVSPRLQLSNGNRTLTLFNVTRNDTASYKCET QNPVSARRSDSVILNVLYGPDAPTISPLNTSYRSGENLNLSCHAASNPPAQYSWFVNGTFQQSTQELFIPNIT VNNSGSYTCQAHNSDTGLNRTTVTTITVYAEPPKPFITSNNSNPVEDEDAVALTCEPEIQNTTYLWWVNNQS LPVSPRLQLSNDNRTLTLLSVTRNDVGPYECGIQNELSVDHSDPVILNVLYGPDDPTISPSYTYYRPGVNLSL SCHAASNPPAQYSWLIDGNIQQHTQELFISNITEKNSGLYTCQANNSASGHSRTTVKTITVSAELPKPSISSNN SKPVEDKDAVAFTCEPEAQNTTYLWWVNGQSLPVSPRLQLSNGNRTLTLFNVTRNDARAYVCGIQNSVSA NRSDPVTLDVLYGPDTPIISPPDSSYLSGANLNLSCHSASNPSPQYSWRINGIPQQHTQVLFIAKITPNNNGTY ACFVSNLATGRNNSIVKSITV corresponding to amino acids 1-674 of CEA5_HUMAN (SEQ ID NO:863), which also corresponds to amino acids 1-674 of HUMCEA_PEA_{—}1_P7 (SEQ ID NO:866), and a second amino acid sequence being at least 90% homologous to SAGATVGIMIGVLVGVALI corresponding to amino acids 684-702 of CEA5_HUMAN (SEQ ID NO:863), which also corresponds to amino acids 675-693 of HUMCEA_PEA_{—}1_P7 (SEQ ID NO:866), wherein said first amino acid sequence and second amino acid sequence are contiguous and in a sequential order.

According to preferred embodiments of the present invention, there is provided an isolated chimeric polypeptide encoding for an edge portion of HUMCEA_PEA_{—}1_P7 (SEQ ID NO:866), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise VS, having a structure as follows: a sequence starting from any of amino acid numbers 674−x to 674; and ending at any of amino acid numbers 675+((n−2)−x), in which x varies from 0 to n−2.

According to preferred embodiments of the present invention, there is provided an isolated chimeric polypeptide encoding for HUMCEA_PEA_{—}1_P10 (SEQ ID NO:867), comprising a first amino acid sequence being at least 90% homologous to MESPSAPPHRWCIPWQRLLLTASLLTFWNPPTTAKLTIESTPFNVAEGKEVLLLVHNLPQHLFGYSWYKGER VDGNRQIIGYVIGTQQATPGPAYSGREIIYPNASLLIQNIIQNDTGFYTLHVIKSDLVNEEATGQFRVYPELPK PSISSNNSKPVEDKDAVAFTCEPETQDATYLWWVNNQSLPVSPRLQLSNGNRTLTLFNVTRNDTASYKCET QNPVSARRSDS corresponding to amino acids 1-228 of CEA5_HUMAN (SEQ ID NO:863), which also corresponds to amino acids 1-228 of HUMCEA_PEA_{—}1_P10 (SEQ ID NO:867), and a second amino acid sequence being at least 90% homologous to VILNVLYGPDDPTISPSYTYYRPGVNLSLSCHAASNPPAQYSWLIDGNIQQHTQELFISNITEKNSGLYTCQA NNSASGHSRTTVKTITVSAELPKPSISSNNSKPVEDKDAVAFTCEPEAQNTTYLWWVNGQSLPVSPRLQLSN GNRTLTLFNVTRNDARAYVCGIQNSVSANRSDPVTLDVLYGPDTPIISPPDSSYLSGANLNLSCHSASNPSPQ YSWRINGIPQQHTQVLFIAKITPNNNGTYACFVSNLATGRNNSIVKSITVSASGTSPGLSAGATVGIMIGVLV GVALI corresponding to amino acids 1-228 of CEA5_HUMAN (SEQ ID NO:863), which also corresponds to amino acids 229-524 of HUMCEA_PEA_{—}1_P10 (SEQ ID NO:867), wherein said first amino acid sequence and second amino acid sequence are contiguous and in a sequential order.

According to preferred embodiments of the present invention, there is provided an isolated chimeric polypeptide encoding for an edge portion of HUMCEA_PEA_{—}1_P10 (SEQ ID NO:867), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise SV, having a structure as follows: a sequence starting from any of amino acid numbers 228−x to 228; and ending at any of amino acid numbers 229+((n−2)−x), in which x varies from 0 to n−2.

According to preferred embodiments of the present invention, there is provided an isolated chimeric polypeptide encoding for HUMCEA_PEA_{—}1_P19 (SEQ ID NO:869), comprising a first amino acid sequence being at least 90% homologous to MESPSAPPHRWCIPWQRLLLTASLLTFWNPPTTAKLTIESTPFNVAEGKEVLLLVHNLPQHLFGYSWYKGER VDGNRQIIGYVIGTQQATPGPAYSGREIIYPNASLLIQNIIQNDTGFYTLHVIKSDLVNEEATGQFRVYPELPK PSISSNNSKPVEDKDAVAFTCEPETQDATYLWWVNNQSLPVSPRLQLSNGNRTLTLFNVTRNDTASYKCET QNPVSARRSDSVILN corresponding to amino acids 1-232 of CEA5_HUMAN (SEQ ID NO:863), which also corresponds to amino acids 1-232 of HUMCEA_PEA_{—}1_P19 (SEQ ID NO:869), and a second amino acid sequence being at least 90% homologous to VLYGPDTPIISPPDSSYLSGANLNLSCHSASNPSPQYSWRINGIPQQHTQVLFIAKITPNNNGTYACFVSNLAT GRNNSIVKSITVSASGTSPGLSAGATVGIMIGVLVGVALI corresponding to amino acids 589-702 of CEA5_HUMAN (SEQ ID NO:863), which also corresponds to amino acids 233-346 of HUMCEA_PEA_{—}1_P19 (SEQ ID NO:869), wherein said first amino acid sequence and second amino acid sequence are contiguous and in a sequential order.

According to preferred embodiments of the present invention, there is provided an isolated chimeric polypeptide encoding for an edge portion of HUMCEA_PEA_{—}1_P19 (SEQ ID NO:869), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise NV, having a structure as follows: a sequence starting from any of amino acid numbers 232−x to 232; and ending at any of amino acid numbers 233+((n−2)−x), in which x varies from 0 to n−2.

According to preferred embodiments of the present invention, there is provided an isolated chimeric polypeptide encoding for HUMCEA_PEA_{—}1_P20 (SEQ ID NO:870), comprising a first amino acid sequence being at least 90% homologous to MESPSAPPHRWCIPWQRLLLTASLLTFWNPPTTAKLTIESTPFNVAEGKEVLLLVHNLPQHLFGYSWYKGER VDGNRQIIGYVIGTQQATPGPAYSGREIIYPNASLLIQNIIQNDTGFYTLHVIKSDLVNEEATGQFRVYP corresponding to amino acids 1-142 of CEA5_HUMAN (SEQ ID NO:863), which also corresponds to amino acids 1-142 of HUMCEA_PEA_{—}1_P20 (SEQ ID NO:870), and a second amino acid sequence being at least 90% homologous to ELPKPSISSNNSKPVEDKDAVAFTCEPEAQNTTYLWWVNGQSLPVSPRLQLSNGNRTLTLFNVTRNDARAY VCGIQNSVSANRSDPVTLDVLYGPDTPIISPPDSSYLSGANLNLSCHSASNPSPQYSWRINGIPQQHTQVLFIA KITPNNNGTYACFVSNLATGRNNSIVKSITVSASGTSPGLSAGATVGIMIGVLVGVALI corresponding to amino acids 499-702 of CEA5_HUMAN (SEQ ID NO:863), which also corresponds to amino acids 143-346 of HUMCEA_PEA_{—}1_P20 (SEQ ID NO:870), wherein said first amino acid sequence and second amino acid sequence are contiguous and in a sequential order.

According to preferred embodiments of the present invention, there is provided an isolated chimeric polypeptide encoding for an edge portion of HUMCEA_PEA_{—}1_P20 (SEQ ID NO:870), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise PE, having a structure as follows: a sequence starting from any of amino acid numbers 142−x to 142; and ending at any of amino acid numbers 143+((n−2)−x), in which x varies from 0 to n−2.

According to preferred embodiments of the present invention, there is provided an isolated chimeric polypeptide encoding for HUMCACH1A_PEA_{—}1_P7 (SEQ ID NO:796), comprising a first amino acid sequence being at least 90% homologous to MPTSETESVNTENVSGEGENRGCCGSL corresponding to amino acids 466-492 of CCAD_HUMAN_V3 (SEQ ID NO:791), which also corresponds to amino acids 1-27 of HUMCACH1A_PEA_{—}1_P7 (SEQ ID NO:796), a second amino acid sequence being at least 70%, optionally at least 80%, preferably at least 85%, more preferably at least 90% and most preferably at least 95% homologous to a polypeptide having the sequence WCWWRRRGAAKAGPSGCRRWG corresponding to amino acids 28-48 of HUMCACH1A_PEA_{—}1_P7 (SEQ ID NO:796), and a third amino acid sequence being at least 90% homologous to QAISKSKLSRRWRRWNRFNRRRCRAAVKSVTFYWLVIVLVFLNTLTISSEHYNQPDWLTQIQDIANKVLLA LFTCEMLVKMYSLGLQAYFVSLFNRFDCFVVCGGITETILVELEIMSPLGISVFRCVRLLRIFKVTRHWTSLS NLVASLLNSMKSIASLLLLLFLFIIIFSLLGMQLFGGKFNFDETQTKRSTFDNFPQALLTVFQILTGEDWNAVM YDGIMAYGGPSSSGMIVCIYFIILFICGNYILLNVFLAIAVDNLADAESLNTAQKEEAEEKERKKIARKESLEN KKNNKPEVNQIANSDNKVTIDDYREEDEDKDPYPPCDVPVGEEEEEEEEDEPEVPAGPRPRRISELNMKEKI APIPEGSAFFILSKTNPIRVGCHKLINHHIFTNLILVFIMLSSAALAAEDPIRSHSFRNTILGYFDYAFTAIFTVEI LLKMTTFGAFLHKGAFCRNYFNLLDMLVVGVSLVSFGIQSSAISVVKILRVLRVLRPLRAINRAKGLKHVVQ CVFVAIRTIGNIMIVTTLLQFMFACIGVQLFKGKFYRCTDEAKSNPEECRGLFILYKDGDVDSPVVRERIWQN SDFNFDNVLSAMMALFTVSTFEGWPALLYKAIDSNGENIGPIYNHRVEISIFFIIYIIIVAFFMMNIFVGFVIVTF QEQGEKEYKNCELDKNQRQCVEYALKARPLRRYIPKNPYQYKFWYVVNSSPFEYMMFVLIMLNTLCLAM QHYEQSKMFNDAMDILNMVFTGVFTVEMVLKVIAFKPKGYFSDAWNTFDSLIVIGSIIDVALSEADPTESEN VPVPTATPGNSEESNRISITFFRLFRVMRLVKLLSRGEGIRTLLWTFIKSFQALPYVALLIAMLFFIYAVIGMQ MFGKVAMRDNNQINRNNNFQTFPQAVLLLFRCATGEAWQEIMLACLPGKLCDPESDYNPGEEYTCGSNFAI VYFISFYMLCAFLIINLFVAVIMDNFDYLTRDWSILGPHHLDEFKRIWSEYDPEAKGRIKHLDVVTLLRRIQPP LGFGKLCPHRVACKRLVAMNMPLNSDGTVMFNATLFALVRTALKIKTEGNLEQANEELRAVIKKIWKKTS MKLLDQVVPPAGDDEVTVGKFYATFLIQDYFRKFKKRKEQGLVGKYPAKNTTIALQAGLRTLHDIGPEIRR AISCDLQDDEPEETKREEEDDVFKRNGALLGNHVNHVNSDRRDSLQQTNTTHRPLHVQRPSIPPASDTEKPL FPPAGNSVCHNHHNHNSIGKQVPTSTNANLNNANMSKAAHGKRPSIGNLEHVSENGHHSSHKHDREPQRR SSVKRTRYYETYIRSDSGDEQLPTICREDPEIHGYFRDPHCLGEQEYFSSEECYEDDSSPTWSRQNYGYYSRY PGRNIDSERPRGYHHPQGFLEDDDSPVCYDSRRSPRRRLLPPTPASHRRSSFNFECLRRQSSQEEVPSSPIFPH RTALPLHLMQQQIMAVAGLDSSKAQKYSPSHSTRSWATPPATPPYRDWTPCYTPLIQVEQSEALDQVNGSL PSLHRSSWYTDEPDISYRTFTPASLTVPSSFRNKNSDKQRSADSLVEAVLISEGLGRYARDPKFVSATKHETA DACDLTIDEMESAASTLLNGNVRPRANGDVGPLSHRQDYELQDFGPGYSDEEPDPGRDEEDLADEMICITTL corresponding to amino acids 494-2161 of CCAD_HUMAN_V3 (SEQ ID NO:791), which also corresponds to amino acids 49-1716 of HUMCACH1A_PEA_{—}1_P7 (SEQ ID NO:796), wherein said first amino acid sequence, second amino acid sequence and third amino acid sequence are contiguous and in a sequential order.

According to preferred embodiments of the present invention, there is provided an isolated polypeptide encoding for an edge portion of HUMCACH1A_PEA_{—}1_P7 (SEQ ID NO:796), comprising an amino acid sequence being at least 70%, optionally at least about 80%, preferably at least about 85%, more preferably at least about 90% and most preferably at least about 95% homologous to the sequence encoding for WCWWRRRGAAKAGPSGCRRWG, corresponding to HUMCACH1A_PEA_{—}1_P7 (SEQ ID NO:796).

According to preferred embodiments of the present invention, there is provided a bridge portion of HUMCACH1A_PEA_{—}1_P7 (SEQ ID NO:796), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise L, having a structure as follows (numbering according to HUMCACH1A_PEA_{—}1_P7 (SEQ ID NO:796)): a sequence starting from any of amino acid numbers 492−x to 492; and ending at any of amino acid numbers 28+((n−2)−x), in which x varies from 0 to n−2.

According to preferred embodiments of the present invention, there is provided an isolated chimeric polypeptide encoding for HUMCACH1A_PEA_{—}1_P13 (SEQ ID NO:802), comprising a first amino acid sequence being at least 70%, optionally at least 80%, preferably at least 85%, more preferably at least 90% and most preferably at least 95% homologous to a polypeptide having the sequence MLRPRCLLRRTAHPPHSAPAPAPARSKCLGSWSNVLIRESSVWSLRL (SEQ ID NO:1477) corresponding to amino acids 1-47 of HUMCACH1A_PEA_{—}1_P13 (SEQ ID NO:802), and a second amino acid sequence being at least 90% homologous to DDEVTVGKFYATFLIQDYFRKFKKRKEQGLVGKYPAKNTTIALQAGLRTLHDIGPEIRRAISCDLQDDEPEE TKREEEDDVFKRNGALLGNHVNHVNSDRRDSLQQTNTTHRPLHVQRPSIPPASDTEKPLFPPAGNSVCHNH HNHNSIGKQVPTSTNANLNNANMSKAAHGKRPSIGNLEHVSENGHHSSHKHDREPQRRSSVKRTRYYETYI RSDSGDEQLPTICREDPEIHGYFRDPHCLGEQEYFSSEECYEDDSSPTWSRQNYGYYSRYPGRNIDSERPRGY HHPQGFLEDDDSPVCYDSRRSPRRRLLPPTPASHRRSSFNFECLRRQSSQEEVPSSPIFPHRTALPLHLMQQQI MAVAGLDSSKAQKYSPSHSTRSWATPPATPPYRDWTPCYTPLIQVEQSEALDQVNGSLPSLHRSSWYTDEP DISYRTFTPASLTVPSSFRNKNSDKQRSADSLVEAVLISEGLGRYARDPKFVSATKHEIADACDLTIDEMESA ASTLLNGNVRPRANGDVGPLSHRQDYELQDFGPGYSDEEPDPGRDEEDLADEMICITTL corresponding to amino acids 1598-2161 of CCAD_HUMAN (SEQ ID NO:790), which also corresponds to amino acids 48-611 of HUMCACH1A_PEA_{—}1_P13 (SEQ ID NO:802), wherein said first amino acid sequence and second amino acid sequence are contiguous and in a sequential order.

According to preferred embodiments of the present invention, there is provided an isolated polypeptide encoding for a head of HUMCACH1A_PEA_{—}1_P13 (SEQ ID NO:802), comprising a polypeptide being at least 70%, optionally at least about 80%, preferably at least about 85%, more preferably at least about 90% and most preferably at least about 95% homologous to the sequence MLRPRCLLRRTAHPPHSAPAPAPARSKCLGSWSNVLIRESSVWSLRL (SEQ ID NO:1477) of HUMCACH1A_PEA_{—}1_P13 (SEQ ID NO:802).

According to preferred embodiments of the present invention, there is provided an isolated chimeric polypeptide encoding for HUMCACH1A_PEA_{—}1_P14 (SEQ ID NO:803), comprising a first amino acid sequence being at least 90% homologous to MSKAAHGKRPSIGNLEHVSENGHHSSHKHDREPQRRSSVKRTRYYETYIRSDSGDEQLPTICREDPEIHGYF RDPHCLGEQEYFSSEECYEDDSSPTWSRQNYGYYSRYPGRNIDSERPRGYHHPQGFLEDDDSPVCYDSRRSP RRRLLPPTPASHRRSSFNFECLRRQSSQEEVPSSPIFPHRTALPLHLMQQQIMAVAGLDSSKAQKYSPSHSTRS WATPPATPPYRDWTPCYTPLIQVEQSEALDQVNGSLPSLHRSSWYTDEPDISYRTFTPASLTVPSSFRNKNSD KQRSADSLVEAVLISEGLGRYARDPKFVSATKHEIADACDLTIDEMESAASTLLNGNVRPRANGDVGPLSH RQDYELQDFGPGYSDEEPDPGRDEEDLADEMICITTL corresponding to amino acids 1763-2161 of CCAD_HUMAN (SEQ ID NO:790), which also corresponds to amino acids 1-399 of HUMCACH1A_PEA_{—}1_P14 (SEQ ID NO:803).

According to preferred embodiments of the present invention, there is provided an isolated chimeric polypeptide encoding for HUMCACH1A_PEA_{—}1_P17 (SEQ ID NO:805), comprising a first amino acid sequence being at least 90% homologous to MMMMMMMKKMQHQRQQQADHANEANYARGTRLPLSGEGPTSQPNSSKQTVLSWQAAIDAARQAKAA QTMSTSAPPPVGSLSQRKRQQYAKSKKQGNSSNSRPARALFCLSLNNPIRRACISIVEWKPFDIFILLAIFANC VALAIYIPFPEDDSNSTNHNLEKVEYAFLIIFTVETFLKIIAYGLLLHPNAYVRNGWNLLDFVIVIVGLFSVILE QLTKETEGGNHSSGKSGGFDVKALRAFRVLRPLRLVSGVPSLQVVLNSIIKAMVPLLHIALLVLFVIIIYAIIG LELFIGKMHKTCFFADSDIVAEEDPAPCAFSGNGRQCTANGTECRSGWVGPNGGITNFDNFAFAMLTVFQCI TMEGWTDVLYWMNDAMGFELPWVYFVSLVIFGSFFVLNLVLGVLSG corresponding to amino acids 1-407 of CCAD_HUMAN (SEQ ID NO:790), which also corresponds to amino acids 1-407 of HUMCACH1A_PEA_{—}1_P17 (SEQ ID NO:805), and a second amino acid sequence being at least 70%, optionally at least 80%, preferably at least 85%, more preferably at least 90% and most preferably at least 95% homologous to a polypeptide having the sequence HGGSRL (SEQ ID NO:1478) corresponding to amino acids 408-413 of HUMCACH1A_PEA_{—}1_P17 (SEQ ID NO:805), wherein said first amino acid sequence and second amino acid sequence are contiguous and in a sequential order.

According to preferred embodiments of the present invention, there is provided an isolated polypeptide encoding for a tail of HUMCACH1A_PEA_{—}1_P17 (SEQ ID NO:805), comprising a polypeptide being at least 70%, optionally at least about 80%, preferably at least about 85%, more preferably at least about 90% and most preferably at least about 95% homologous to the sequence HGGSRL (SEQ ID NO:1478) in HUMCACH1A_PEA_{—}1_P17 (SEQ ID NO:805).

According to preferred embodiments of the present invention, there is provided an isolated chimeric polypeptide encoding for AA583399_PEA_{—}1_P2 (SEQ ID NO:684), comprising a first amino acid sequence being at least 90% homologous to MFTRQAGHFVEGSKAGRSRGRLCLSQALRVAVRGAFVSLWFAAGAGDRERNKGDKGAQTGAGLSQEAED VDVSRARRVTDAPQGTLCGTGNRNSGSQSARVVGVAHLGEAFRVGVEQAISSCPEEVHGRHGLSMEIMWA RMDVALRSPGRGLLAGAGALCMTLAESSCPDYERGRRACLTLHRHPTPHCSTWGLPLRVAGSWLTVVTVE ALGGWRMGVRRTGQVGPTMHPPPVSGASPLLLHHLLLLLLIIILTC corresponding to amino acids 59-313 of MYEO_HUMAN_V1 (SEQ ID NO:680), which also corresponds to amino acids 1-255 of AA583399_PEA_{—}1_P2 (SEQ ID NO:684).

According to preferred embodiments of the present invention, there is provided an isolated chimeric polypeptide encoding for AA583399_PEA_{—}1_P4 (SEQ ID NO:685), comprising a first amino acid sequence being at least 70%, optionally at least 80%, preferably at least 85%, more preferably at least 90% and most preferably at least 95% homologous to a polypeptide having the sequence MSDLFIGFLVCSLSPLGTGTRCSCSPG (SEQ ID NO:1479) corresponding to amino acids 1-27 of AA583399_PEA_{—}1_P4 (SEQ ID NO:685), and a second amino acid sequence being at least 90% homologous to RNSGSQSARVVGVAHLGEAFRVGVEQAISSCPEEVHGRHGLSMEIMWARMDVALRSPGRGLLAGAGALC MTLAESSCPDYERGRRACLTLHRHPTPHCSTWGLPLRVAGSWLTVVTVEALGGWRMGVRRTGQVGPTMH PPPVSGASPLLLHHLLLLLLIIILTC corresponding to amino acids 150-313 of MYEO_HUMAN_V1 (SEQ ID NO:680), which also corresponds to amino acids 28-191 of AA583399_PEA_{—}1_P4 (SEQ ID NO:685), wherein said first amino acid sequence and second amino acid sequence are contiguous and in a sequential order.

According to preferred embodiments of the present invention, there is provided an isolated polypeptide encoding for a head of AA583399_PEA_{—}1_P4 (SEQ ID NO:685), comprising a polypeptide being at least 70%, optionally at least about 80%, preferably at least about 85%, more preferably at least about 90% and most preferably at least about 95% homologous to the sequence MSDLFIGFLVCSLSPLGTGTRCSCSPG (SEQ ID NO:1479) of AA583399_PEA_{—}1_P4 (SEQ ID NO:685).

According to preferred embodiments of the present invention, there is provided an isolated chimeric polypeptide encoding for AA583399_PEA_{—}1_P5 (SEQ ID NO:686), comprising a first amino acid sequence being at least 90% homologous to MEIMWARMDVALRSPGRGLLAGAGALCMTLAESSCPDYERGRRACLTLHRHPTPHCSTWGLPLRVAGSW LTVVTVEALGGWRMGVRRTGQVGPTMHPPPVSGASPLLLHHLLLLLLIIILTC corresponding to amino acids 192-313 of MYEO_HUMAN_V2 (SEQ ID NO:681), which also corresponds to amino acids 1-122 of AA583399_PEA_{—}1_P5 (SEQ ID NO:686).

According to preferred embodiments of the present invention, there is provided an isolated chimeric polypeptide encoding for AA583399_PEA_{—}1_P10 (SEQ ID NO:689), comprising a first amino acid sequence being at least 90% homologous to MFTRQAGHFVEGSKAGRSRGRLCLSQALRVAVRGAFVSLWFAAGAGDRERNKGDKGAQTGAGLSQEAED VDVSRARRVTDAPQGTLCGTGNRNSGSQSARAVGVAHLGEAFRVGVEQAISSCPEEVHGRHGLSMEIMWA QMDVALRSPGRGLLAGAGALCMTLAESSCPDYERGRRACLTLHRHPTPHCSTWGLPLRVAGSWLTVVTVE ALGRWRMGVRRTGQVGPTMHPPPVSGASPLLLHHLLLLLLIIILTC corresponding to amino acids 59-313 of MYEO_HUMAN_V3 (SEQ ID NO:682), which also corresponds to amino acids 1-255 of AA583399_PEA_{—}1_P10 (SEQ ID NO:689).

According to preferred embodiments of the present invention, there is provided an antibody capable of specifically binding to an epitope of an amino acid sequence as described herein.

Optionally the amino acid sequence corresponds to a bridge, edge portion, tail, head or insertion as described herein.

Optionally the antibody is capable of differentiating between a splice variant having said epitope and a corresponding known protein.

According to preferred embodiments of the present invention, there is provided a kit for detecting colon cancer, comprising a kit detecting overexpression of a splice variant as described herein.

Optionally the kit comprises a NAT-based technology.

Optionally said the kit further comprises at least one primer pair capable of selectively hybridizing to a nucleic acid sequence as described herein. Optionally the kit further comprises at least one oligonucleotide capable of selectively hybridizing to a nucleic acid sequence as described herein. The kit optionally comprises an antibody as described herein. The kit optionally further comprises at least one reagent for performing an ELISA or a Western blot.

There is optionally provided a method for detecting colon cancer, comprising detecting overexpression of a splice variant as described herein. Detecting overexpression is optionally performed with a NAT-based technology.

Optionally s detecting overexpression is performed with an immunoassay, optionally wherein said immunoassay comprises an antibody as described herein. A biomarker capable of detecting colon cancer, comprising any of the above nucleic acid sequences or a fragment thereof, or any of the above amino acid sequences or a fragment thereof. A method for screening for colon cancer, comprising detecting colon cancer cells with a biomarker or an antibody or a method or assay as described herein. A method for diagnosing colon cancer, comprising detecting colon cancer cells with a biomarker or an antibody or a method or assay as described herein. A method for monitoring disease progression and/or treatment efficacy and/or relapse of colon cancer, comprising detecting colon cancer cells with a biomarker or an antibody or a method or assay as described herein. A method of selecting a therapy for colon cancer, comprising detecting colon cancer cells with a biomarker or an antibody or a method or assay as described herein and selecting a therapy according to said detection.

According to preferred embodiments of the present invention, preferably any of the above nucleic acid and/or amino acid sequences further comprises any sequence having at least about 70%, preferably at least about 80%, more preferably at least about 90%, most preferably at least about 95% homology thereto.

Unless otherwise noted, all experimental data relates to variants of the present invention, named according to the segment being tested (as expression was tested through RT-PCR as described).

All nucleic acid sequences and/or amino acid sequences shown herein as embodiments of the present invention relate to their isolated form, as isolated polynucleotides (including for all transcripts), oligonucleotides (including for all segments, amplicons and primers), peptides (including for all tails, bridges, insertions or heads, optionally including other antibody epitopes as described herein) and/or polypeptides (including for all proteins). It should be noted that oligonucleotide and polynucleotide, or peptide and polypeptide, may optionally be used interchangeably.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein. As used herein, the following terms have the meanings ascribed to them unless specified otherwise.

### BRIEF DESCRIPTION OF DRAWINGS

FIG. 1. is schematic summary of cancer biomarkers selection engine and the wet validation stages.

FIG. 2. Schematic illustration, depicting grouping of transcripts of a given cluster based on presence or absence of unique sequence regions.

FIG. 3 is schematic summary of quantitative real-time PCR analysis.

FIG. 4 is schematic presentation of the oligonucleotide based microarray fabrication.

FIG. 5 is schematic summary of the oligonucleotide based microarray experimental flow.

FIG. 6 is a histogram showing Cancer and cell-line vs. normal tissue expression for Cluster M85491.

FIG. 7 is a histogram showing expression of the Ephrin type-B receptor 2 precursor (EC 2.7.1.112) (Tyrosine-protein kinase receptor EPH-3) M85491 transcripts which are detectable by amplicon as depicted in sequence name M85491seg24 (SEQ ID NO:1276) in normal and cancerous colon tissues.

FIG. 8 is a histogram showing the expression of M85491 transcripts which are detectable by amplicon as depicted in sequence name M85491seg24 (SEQ ID NO:1276) in different normal issues.

FIG. 9 is histogram, showing Cancer and cell-line vs. normal tissue expression for Cluster T10888, demonstrating overexpression in colorectal cancer, a mixture of malignant tumors from different tissues, pancreas carcinoma and gastric carcinoma.

FIG. 10 is a histogram showing expression of the CEA6_HUMAN Carcinoembryonic antigen-related cell adhesion molecule 6 (T10888) transcripts which are detectable by amplicon as depicted in sequence name T10888 junc11-17 (SEQ ID NO: 1279), in normal and cancerous colon tissues.

FIG. 11 is a the histogram showing the expression of T10888 transcripts, which are detectable by amplicon as depicted in sequence name T10888junc11-17 (SEQ ID NO: 1282), in different normal tissues.

FIG. 12 is a histogram showing Cancer and cell-line vs. normal tissue expression for Cluster H14624.

FIG. 13 is a histogram, showing Cancer and cell-line vs. normal tissue expression for Cluster H53626, demonstrating overexpression in the epithelial malignant tumors, a mixture of malignant tumors from different tissues and myosarcoma.

FIG. 14 is a histogram showing expression of the above-indicated Homo sapiens fibroblast growth factor receptor-like 1 (FGFRL1) H53626 transcripts, which are detectable by amplicon as depicted in sequence name H53626 junc24-27F1R3 (SEQ ID NO: 1285), in normal and cancerous colon tissues.

FIG. 15 is the expression of Homo sapiens fibroblast growth factor receptor-like 1 (FGFRL1) H53626 transcripts, which are detectable by amplicon as depicted in sequence name H53626seg25 (SEQ ID NO: 1288), in normal and cancerous colon tissues.

FIG. 16 is a a histogram, showing Cancer and cell-line vs. normal tissue expression for Cluster HSENA78, demonstrating overexpression in the epithelial malignant tumors and lung malignant tumors.

FIG. 17 is a histogram, showing Cancer and cell-line vs. normal tissue expression for the Cluster HUMODCA, demonstrating overexpression in the brain malignant tumors, colorectal cancer, epithelial malignant tumors and a mixture of malignant tumors from different tissues.

FIG. 18 is a histogram, showing Cancer and cell-line vs. normal tissue expression for the cluster R00299, demonstratin overexpression in the lung malignant tumors.

FIG. 19 is the histograms showing Cancer and cell-line vs. normal tissue expression for the cluster Z44808, demonstrating overexpression in the colorectal cancer, lung cancer and pancreas carcinoma.

FIG. 20 is the histograms showing Cancer and cell-line vs. normal tissue expression for the cluster Z25299, demonstrating overexpression in the brain malignant tumors, a mixture of malignant tumors from different tissues and ovarian carcinoma.

FIG. 21 is a histogram showing expression of Z25299 transcripts, which are detectable by amplicon as depicted in sequence name Z25299seg20 (SEQ ID NO: 1294), in normal and cancerous colon tissues.

FIG. 22 is a histogram showing the expression of Secretory leukocyte protease inhibitor Acid-stable proteinase inhibitor with strong affinities for trypsin, chymotrypsin, elastase, and cathepsin G. May prevent elastase-mediated damage to oral and possibly other mucosal tissues Z25299 transcripts which are detectable by amplicon as depicted in sequence name Z25299seg20 (SEQ ID NO: 1294) in different normal tissues.

FIG. 23 is the histograms showing Cancer and cell-line vs. normal tissue expression for the cluster HUMANK, demonstrating overexpression in epithelial malignant tumors.

FIG. 24 is the histograms showing Cancer and cell-line vs. normal tissue expression for the cluster HUMCA1XIA, demonstrating overexpression in the bone malignant tumors, epithelial malignant tumors, a mixture of malignant tumors from different tissues and lung malignant tumors.

FIG. 25 is the histograms showing Cancer and cell-line vs. normal tissue expression for the cluster HSS100PCB, demonstrating overexpression in the mixture of malignant tumors from different tissues.

FIG. 26 is the histograms showing Cancer and cell-line vs. normal tissue expression for the cluster D11853, demonstrating overexpression in the brain malignant tumors, colorectal cancer and a mixture of malignant tumors from different tissues.

FIG. 27 is the histograms showing Cancer and cell-line vs. normal tissue expression for the cluster R11723, demonstrating overexpression in the epithelial malignant tumors, a mixture of malignant tumors from different tissues and kidney malignant tumors

FIG. 28 is the histogram showing expression of the R11723 transcripts, which are detectable by amplicon as depicted in sequence name R11723 seg13 (SEQ ID NO: 1297) in normal and cancerous colon tissues.

FIG. 29 is the histogram showing expression of the R11723 transcripts, which are detectable by amplicon as depicted in sequence name R11723 junc11-18 (SEQ ID NO: 1300) in normal and cancerous colon tissues.

FIG. 30 is the histogram showing the expression of R11723 transcripts, detectable by amplicon depicted in sequence name R11723seg13 (SEQ ID NO: 1297) in different normal tissues.

FIG. 31 is the histogram showing the expression of R11723 transcripts, detectable by amplicon in sequence name R11723 junc11-18 (SEQ ID NO: 1300) in different normal tissues.

FIG. 32 is a histogram showing over expression of the SMO2_HUMAN SPARC related modular calcium-binding protein 2 precursor (Secreted modular calcium-binding protein 2) (SMOC-2) (Smooth muscle-associated protein 2) Z44808 transcripts which are detectable by amplicon as depicted in sequence name Z44808junc8-11 (SEQ ID NO: 1291) in cancerous colon samples relative to the normal samples

FIG. 33 is the histograms showing Cancer and cell-line vs. normal tissue expression for the cluster M77903, demonstrating overexpression in ovarian carcinoma and uterine malignancies.

FIG. 34 is the histogram showing expression of the SSR-alpha M77903 transcripts, which are detectable by amplicon, as depicted in sequence name M77903seg18 (SEQ ID NO: 1303) in normal and cancerous colon tissues.

FIG. 35 is the histogram showing low over expression for amplicon M77903 junc20-34-35 (SEQ ID NO: 1309) in the experiment carried out with colon.

FIG. 36 is the histogram showing low over expression for amplicon M77903 junc20-28 (SEQ ID NO: 1306) in the experiment carried out with colon

FIGS. 37-38 are histograms showing differential expression of 6 sequences: (M85491seg24 (SEQ ID NO: 1276), M77903 seg18 (SEQ ID NO: 1303), M77903junc20-28 (SEQ ID NO: 1306), Z44808 junc8-11 (SEQ ID NO: 1291), Z25299 seg 20 (SEQ ID NO: 1294) and HSKITCR seg3 (SEQ ID NO: 1309) in normal and cancerous colon tissues, in different combinations.

FIG. 39 is a histogram showing the expression of SMO2_HUMAN SPARC related modular calcium-binding protein 2 precursor (Secreted modular calcium-binding protein 2) (SMOC-2) (Smooth muscle-associated protein 2) Z44808 transcripts which are detectable by amplicon as depicted in sequence name Z44808 junc8-11 (SEQ ID NO: 1291) in different normal tissues.

FIG. 40 is the histogram showing Cancer and cell-line vs. normal tissue expression for the cluster AA583399, demonstrating overexpression in brain malignant tumors, epithelial malignant tumors, a mixture of malignant tumors from different tissues and gastric carcinoma.

FIG. 41 is the histogram showing expression of the AA583399 transcripts, which are detectable by amplicon as depicted in sequence name AA583399seg30-32 (SEQ ID NO: 1321), in normal and cancerous colon tissues.

FIG. 42 is the histogram showing expression of the AA583399 transcripts which are detectable by amplicon as depicted in sequence name AA583399seg17 (SEQ ID NO: 1324) in normal and cancerous colon tissues.

FIG. 43 is the histogram showing expression of the AA583399 transcripts which are detectable by amplicon as depicted in sequence name AA583399seg1 (SEQ ID NO: 1327) in normal and cancerous colon tissues.

FIG. 44 is the histogram showing Cancer and cell-line vs. normal tissue expression for the cluster AI684092, demonstrating overexpression in brain malignant tumors, epithelial malignant tumors and a mixture of malignant tumors from different tissues.

FIG. 45 is the histogram showing expression of the AA5315457 transcripts which are detectable by amplicon as depicted in sequence name AA5315457seg8 (SEQ ID NO: 1330) in normal and cancerous colon tissues.

FIG. 46 is the histogram showing Cancer and cell-line vs. normal tissue expression for the cluster HUMCACH1A, demonstrating overexpression in a mixture of malignant tumors from different tissues.

FIG. 47 is the histogram showing expression of the Voltage-dependent L-type calcium channel alpha-1D subunit Calcium channel, L type, alpha-1 polypeptide, isoform 2 Transcripts, which are detectable by seg 113, 35, 109, 125, in normal and cancerous colon tissues.

FIG. 48 is the histogram showing expression of the HUMCACH1A Transcripts, which are detectable by amplicon as depicted in sequence name HUMCACH1Aseg101 (SEQ ID NO: 1337), in normal and cancerous colon tissues.

FIG. 49 is the histogram showing Cancer and cell-line vs. normal tissue expression for the cluster HUMCEA, demonstrating overexpression in epithelial malignant tumors, a mixture of malignant tumors from different tissues and pancreas carcinoma.

FIG. 50 is the histogram showing expression of the HUMCEA transcripts which are detectable by seg12 and seg9, in normal and cancerous colon tissues.

FIG. 51 is the histogram showing expression of the Carcinoembryonic antigen-related cell adhesion molecule 5 CEACAM5 HUMCEA transcripts which are detectable by amplicon as depicted in sequence name HUMCEA seg31 (SEQ ID NO: 1342) in normal and cancerous colon tissues.

FIG. 52 is the histogram showing expression of the Carcinoembryonic antigen-related cell adhesion molecule 5 CEACAM5 HUMCEA transcripts which are detectable by amplicon as depicted in sequence name HUMCEA seg33 (SEQ ID NO: 1345) in normal and cancerous colon tissues.

FIG. 53 is the histogram showing expression of the Carcinoembryonic antigen-related cell adhesion molecule 5 CEACAM5 HUMCEA transcripts which are detectable by amplicon as depicted in sequence name HUMCEA seg35 (SEQ ID NO: 1348) in normal and cancerous colon tissues.

FIG. 54 is the histogram showing Cancer and cell-line vs. normal tissue expression for the cluster M78035, demonstrating overexpression in brain malignant tumors, colorectal cancer, epithelial malignant tumors, a mixture of malignant tumors from different tissues, malignant tumors involving the lymph nodes and pancreas carcinoma.

FIG. 55 is the histogram showing expression of the S-adenosylhomocysteine hydrolase (AHCY) M78035 transcripts, which are detectable by amplicon as depicted in sequence name M78035seg42 (SEQ ID NO: 1351), in normal and cancerous colon tissues

FIG. 56 is the histogram showing Cancer and cell-line vs. normal tissue expression for the cluster R30650, demonstrating overexpression in epithelial malignant tumors and a mixture of malignant tumors from different tissues.

FIG. 57 is the histogram showing expression of the R30650 transcripts which are detectable by amplicon as depicted in sequence name R30650 seg76 (SEQ ID NO: 1354) in normal and cancerous colon tissues.

FIG. 58 is the histogram showing Cancer and cell-line vs. normal tissue expression for the cluster T23657, demonstrating overexpression in epithelial malignant tumors.

FIG. 59 is the histogram showing expression of solute carrier organic anion transporter family, member 4A1 (SLCO4A1) T23657 transcripts, which are detectable by amplicon as depicted in sequence name T23657 seg17-18 (SEQ ID NO: 1357), in normal and cancerous colon tissues.

FIG. 60 is the histogram showing expression of solute carrier organic anion transporter family, member 4A1 (SLCO4A1) T23657 transcripts, which are detectable by amplicon as depicted in sequence name T23657 seg22 (SEQ ID NO: 1360), in normal and cancerous colon tissues.

FIG. 61 is the histogram showing expression of solute carrier organic anion transporter family, member 4A1 (SLCO4A1) T23657 transcripts, which are detectable by amplicon as depicted in sequence name T23657 seg29-32 (SEQ ID NO: 1363), in normal and cancerous colon tissues.

FIG. 62 is the histogram showing expression of solute carrier organic anion transporter family, member 4A1 (SLCO4A1) T23657 transcripts, which are detectable by amplicon as depicted in sequence name T23657 seg41 (SEQ ID NO: 1366), in normal and cancerous colon tissues.

FIG. 63 is the histogram showing Cancer and cell-line vs. normal tissue expression for the cluster T51958, demonstrating overexpression in epithelial malignant tumors and a mixture of malignant tumors from different tissues.

FIG. 64 is the histogram showing expression of PTK7 protein tyrosine kinase 7 (PTK7) T51958 transcripts which are detectable by amplicon as depicted in sequence name T51958seg38 (SEQ ID NO: 1369) in normal and cancerous colon tissues.

FIG. 65 is the histogram showing expression of PTK7 protein tyrosine kinase 7 (PTK7) T51958 transcripts which are detectable by amplicon as depicted in sequence name T51958seg7 (SEQ ID NO: 1372) in normal and cancerous colon tissues.

FIG. 66 is the histogram showing Cancer and cell-line vs. normal tissue expression for the cluster Z17877, demonstrating overexpression in brain malignant tumors and malignant tumors involving the bone marrow.

FIG. 67 is the histogram showing expression of c-myc-P64 mRNA, initiating from promoter P0 Z17877 transcripts, which are detectable by amplicon as depicted in sequence name Z17877seg8 (SEQ ID NO: 1375), in normal and cancerous colon tissues.

FIG. 68 is the histogram showing combined expression of 19 sequences (T23657seg 29 (SEQ ID NO: 1363), T23657seg 22 (SEQ ID NO: 1360), T23657seg 41 (SEQ ID NO: 1366), T23657seg17-18 (SEQ ID NO: 1357), AA315457seg8, R30650seg76 (SEQ ID NO: 1354), HUM-CEASeg 33 (SEQ ID NO: 1345), CEA-Seg35 (SEQ ID NO: 1348), CEA-Seg31 (SEQ ID NO: 1342), AA583399seg1 (SEQ ID NO: 1327), AA583399seg17 (SEQ ID NO: 1324), AA58339-seg30-32 (SEQ ID NO: 1321), HUMCACH1Aseg101 (SEQ ID NO: 1337), HSHCGI seg20 (SEQ ID NO: 1378), HSHCGI seg35 (SEQ ID NO: 1381), M78035seg 42 (SEQ ID NO: 1351), T51958seg7 (SEQ ID NO: 1372), T51958 seg3 (SEQ ID NO: 1369) and, Z17877 seg8 (SEQ ID NO: 1375)) in normal and cancerous colon tissues.

FIG. 69 is the histogram showing expression of TRIM31 tripartite motif HSHCGI transcripts which are detectable by amplicon as depicted in sequence name HSHCGI seg20 (SEQ ID NO: 1378) in normal and cancerous colon tissues.

FIG. 70 is the histogram showing expression of TRIM31 tripartite motif HSHCGI transcripts which are detectable by amplicon as depicted in sequence name HSHCGI seg35 (SEQ ID NO: 1381) in normal and cancerous colon tissues.

FIG. 71 is a histogram showing the expression of fibroblast growth factor receptor-like 1 (FGFRL1) transcripts detectable by or according to H53626 seg25 (SEQ ID NO: 1288) amplicon(s) and H53626 seg25F (SEQ ID NO: 1286) and H53626 seg25R (SEQ ID NO: 1287) in different normal tissues.

FIG. 72 is a histogram showing the expression of fibroblast growth factor receptor-like 1 (FGFRL1) transcripts detectable by or according to H53626 seg25 (SEQ ID NO: 1285) amplicon(s) and H53626 seg25F (SEQ ID NO: 1283) and H53626 junc24-27F1R3 (SEQ ID NO: 1284) in different normal tissues.

FIG. 73 is a histogram showing over expression of the Matrix metalloproteinase 11 (stromelysin 3) (MMP11) transcripts, which are detectable by amplicon as depicted in sequence name HSSTROL3 junc21-27 (SEQ ID NO: 1312), in cancerous colon samples relative to the normal samples.

FIG. 74 is a histogram showing over expression of the Matrix metalloproteinase 11 (stromelysin 3) (MMP11) transcripts, which are detectable by amplicon as depicted in sequence name HSSTROL3 seg25 (SEQ ID NO: 1315), in cancerous colon samples relative to the normal samples.

FIG. 75 is the histogram showing Cancer and cell-line vs. normal tissue expression for the cluster HSSTROL3, demonstrating overexpression in transitional cell carcinoma, epithelial malignant tumors, a mixture of malignant tumors from different tissues and pancreas carcinoma.

FIG. 76 is a histogram showing the expression of Stromelysin-3 HSSTROL3 transcripts, which are detectable by amplicon as depicted in sequence name HSSTROL3 seg24, in different normal tissues.

FIG. 77 is a histogram showing over expression of the Homo sapiens collagen, type XI, alpha 1 (COL11A1) transcripts detectable by seg55—HUMCA1XIA_seg55 amplicon in cancerous Colon samples relative to the normal samples.

FIG. 78 is a histogram showing expression of the Homo sapiens collagen, type XI, alpha 1 (COL11A1) transcripts detectable by seg55—HUMCA1XIA_seg55 amplicon in various normal tissues.

FIG. 79 is a histogram showing over expression of the Homo sapiens collagen, type XI, alpha 1 (COL11A1) transcripts detectable by HUMCA1XIA_seg54-55F2R2 in normal and cancerous Colon tissues.

FIG. 80 is a histogram showing expression of the Homo sapiens collagen, type XI, alpha 1 (COL11A1) transcripts detectable by HUMCA1XIA_seg54-55F2R2 in various normal tissues.

### DESCRIPTION OF PREFERRED EMBODIMENTS

The present invention is of novel markers for colon cancer that are both sensitive and accurate. Biomolecular sequences (amino acid and/or nucleic acid sequences) uncovered using the methodology of the present invention and described herein can be efficiently utilized as tissue or pathological markers and/or as drugs or drug targets for treating or preventing a disease.

These markers are specifically released to the bloodstream under conditions of colon cancer and/or other colon pathology, and/or are otherwise expressed at a much higher level and/or specifically expressed in colon cancer tissue or cells. The measurement of these markers, alone or in combination, in patient samples provides information that the diagnostician can correlate with a probable diagnosis of colon cancer and/or pathology.

The present invention therefore also relates to diagnostic assays for colon cancer and/or colon pathology, and methods of use of such markers for detection of colon cancer and/or colon pathology, optionally and preferably in a sample taken from a subject (patient), which is more preferably some type of blood sample.

In another embodiment, the present invention relates to bridges, tails, heads and/or insertions, and/or analogs, homologs and derivatives of such peptides. Such bridges, tails, heads and/or insertions are described in greater detail below with regard to the Examples.

As used herein a “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding known protein, while at least a second portion of the variant comprises the tail.

As used herein a “head” refers to a peptide sequence at the beginning of an amino acid sequence that is unique to a splice variant according to the present invention. Therefore, a splice variant having such a head may optionally be considered as a chimera, in that at least a first portion of the splice variant comprises the head, while at least a second portion is typically highly homologous (often 100% identical) to a portion of the corresponding known protein.

As used herein “an edge portion” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now contiguous in the splice variant that were not contiguous in the known protein. A “bridge” may optionally be an edge portion as described above, but may also include a join between a bead and a “known protein” portion of a variant, or a join between a tail and a “known protein” portion of a variant, or a join between an insertion and a “known protein” portion of a variant.

Optionally and preferably, a bridge between a tail or a head or a unique insertion, and a “known protein” portion of a variant, comprises at least about 10 amino acids, more preferably at least about 20 amino acids, most preferably at least about 30 amino acids, and even more preferably at least about 40 amino acids, in which at least one amino acid is from the tail/head/insertion and at least one amino acid is from the “known protein” portion of a variant. Also optionally, the bridge may comprise any number of amino acids from about 10 to about 40 amino acids (for example, 10, 11, 12, 13 . . . 37, 38, 39, 40 amino acids in length, or any number in between).

It should be noted that a bridge cannot be extended beyond the length of the sequence in either direction, and it should be assumed that every bridge description is to be read in such manner that the bridge length does not extend beyond the sequence itself.

Furthermore, bridges are described with regard to a sliding window in certain contexts below. For example, certain descriptions of the bridges feature the following format: a bridge between two edges (in which a portion of the known protein is not present in the variant) may optionally be described as follows: a bridge portion of CONTIG-NAME_P1 (representing the name of the protein), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise XX (2 amino acids in the center of the bridge, one from each end of the edge), having a structure as follows (numbering according to the sequence of CONTIG-NAME_P1): a sequence starting from any of amino acid numbers 49−x to 49 (for example); and ending at any of amino acid numbers 50+((n−2)−x) (for example), in which x varies from 0 to n−2. In this example, it should also be read as including bridges in which n is any number of amino acids between 10-50 amino acids in length. Furthermore, the bridge polypeptide cannot extend beyond the sequence, so it should be read such that 49−x (for example) is not less than 1, nor 50+((n−2)−x) (for example) greater than the total sequence length.

In another embodiment, this invention provides antibodies specifically recognizing the splice variants and polypeptide fragments thereof of this invention. Preferably such antibodies differentially recognize splice variants of the present invention but do not recognize a corresponding known protein (such known proteins are discussed with regard to their splice variants in the Examples below).

In another embodiment, this invention provides an isolated nucleic acid molecule encoding for a splice variant according to the present invention, having a nucleotide sequence as set forth in any one of the sequences listed herein, or a sequence complementary thereto. In another embodiment, this invention provides an isolated nucleic acid molecule, having a nucleotide sequence as set forth in any one of the sequences listed herein, or a sequence complementary thereto. In another embodiment, this invention provides an oligonucleotide of at least about 12 nucleotides, specifically hybridizable with the nucleic acid molecules of this invention. In another embodiment, this invention provides vectors, cells, liposomes and compositions comprising the isolated nucleic acids of this invention.

In another embodiment, this invention provides a method for detecting a splice variant according to the present invention in a biological sample, comprising: contacting a biological sample with an antibody specifically recognizing a splice variant according to the present invention under conditions whereby the antibody specifically interacts with the splice variant in the biological sample but do not recognize known corresponding proteins (wherein the known protein is discussed with regard to its splice variant(s) in the Examples below), and detecting said interaction; wherein the presence of an interaction correlates with the presence of a splice variant in the biological sample.

In another embodiment, this invention provides a method for detecting a splice variant nucleic acid sequences in a biological sample, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of a splice variant nucleic acid sequence in the biological sample.

According to the present invention, the splice variants described herein are non-limiting examples of markers for diagnosing colon cancer and/or colon pathology. Each splice variant marker of the present invention can be used alone or in combination, for various uses, including but not limited to, prognosis, prediction, screening, early diagnosis, determination of progression, therapy selection and treatment monitoring of colon cancer and/or colon pathology.

According to optional but preferred embodiments of the present invention, any marker according to the present invention may optionally be used alone or combination. Such a combination may optionally comprise a plurality of markers described herein, optionally including any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker. With regard to such a ratio between any marker described herein (or a combination thereof) and a known marker, more preferably the known marker comprises the “known protein” as described in greater detail below with regard to each cluster or gene.

According to other preferred embodiments of the present invention, a splice variant protein or a fragment thereof, or a splice variant nucleic acid sequence or a fragment thereof, may be featured as a biomarker for detecting colon cancer and/or colon pathology, such that a biomarker may optionally comprise any of the above.

According to still other preferred embodiments, the present invention optionally and preferably encompasses any amino acid sequence or fragment thereof encoded by a nucleic acid sequence corresponding to a splice variant protein as described herein. Any oligopeptide or peptide relating to such an amino acid sequence or fragment thereof may optionally also (additionally or alternatively) be used as a biomarker, including but not limited to the unique amino acid sequences of these proteins that are depicted as tails, heads, insertions, edges or bridges. The present invention also optionally encompasses antibodies capable of recognizing, and/or being elicited by, such oligopeptides or peptides.

The present invention also optionally and preferably encompasses any nucleic acid sequence or fragment thereof, or amino acid sequence or fragment thereof, corresponding to a splice variant of the present invention as described above, optionally for any application.

Non-limiting examples of methods or assays are described below.

The present invention also relates to kits based upon such diagnostic methods or assays.

### Nucleic Acid Sequences and Oligonucleotides

Various embodiments of the present invention encompass nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or artificially induced, either randomly or in a targeted fashion.

The present invention encompasses nucleic acid sequences described herein; fragments thereof, sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth below], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

A “nucleic acid fragment” or an “oligonucleotide” or a “polynucleotide” are used herein interchangeably to refer to a polymer of nucleic acids. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Preferred embodiments of the present invention encompass oligonucleotide probes.

An example of an oligonucleotide probe which can be utilized by the present invention is a single stranded polynucleotide which includes a sequence complementary to the unique sequence region of any variant according to the present invention, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Alternatively, an oligonucleotide probe of the present invention can be designed to hybridize with a nucleic acid sequence encompassed by any of the above nucleic acid sequences, particularly the portions specified above, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases. Preferably, the oligonucleotide of the present invention features at least 17, at least 18, at least 19, at least 20, at least 22, at least 25, at least 30 or at least 40, bases specifically hybridizable with the biomarkers of the present invention.

The oligonucleotides of the present invention may comprise heterocylic nucleosides consisting of purines and the pyrimidines bases, bonded in a 3′ to 5′ phosphodiester linkage.

Preferably used oligonucleotides are those modified at one or more of the backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases particularly useful for increasing the binding affinity of the oligomeric compounds of the invention include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

It will be appreciated that oligonucleotides of the present invention may include further modifications for more efficient use as diagnostic agents and/or to increase bioavailability, therapeutic efficacy and reduce cytotoxicity.

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific, lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters (Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the trasgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

### Hybridization Assays

Detection of a nucleic acid of interest in a biological sample may optionally be effected by hybridization-based assays using an oligonucleotide probe (non-limiting examples of probes according to the present invention were previously described).

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Hybridization based assays which allow the detection of a variant of interest (i.e., DNA or RNA) in a biological sample rely on the use of oligonucleotides which can be 10, 15, 20, or 30 to 100 nucleotides long preferably from 10 to 50, more preferably from 40 to 50 nucleotides long.

Thus, the isolated polynucleotides (oligonucleotides) of the present invention are preferably hybridizable with any of the herein described nucleic acid sequences under moderate to stringent hybridization conditions.

Moderate to stringent hybridization conditions are characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

More generally, hybridization of short nucleic acids (below 200 bp in length, e.g. 17-40 bp in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of radioactive labels include 3H, 14C, 32P, and 35S, Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, FluorX (Amersham) and others [e.g., Kricka et al. (1992), Academic Press San Diego, Calif] can be attached to the oligonucleotides.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods.

As commonly known, radioactive nucleotides can be incorporated into probes of the invention by several methods. Non-limiting examples of radioactive labels include ³H, ¹⁴C, 32P, and 35S.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA), and preferably of DNA.

### NAT Assays

Detection of a nucleic acid of interest in a biological sample may also optionally be effected by NAT-based assays, which involve nucleic acid amplification technology, such as PCR for example (or variations thereof such as real-time PCR for example).

As used herein, a “primer” defines an oligonucleotide which is capable of annealing to (hybridizing with) a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14 Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

It will be appreciated that antisense oligonucleotides may be employed to quantify expression of a splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity.

The polymerase chain reaction and other nucleic acid amplification reactions are well known in the art (various non-limiting examples of these reactions are described in greater detail below). The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Polymerase Chain Reaction (PCR): The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes: see for example Segev, PCT Publication No. WO9001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR/NASBA): The self-sustained sequence replication reaction (3SR) is a transcription-based in vitro amplification system that can exponentially amplify RNA sequences at a uniform temperature. The amplified RNA can then be utilized for mutation detection. In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55 degrees C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (I+X)ⁿ=y, where “X” is the mean efficiency (percent copied in each cycle), “n” is the number of cycles, and “y” is the overall efficiency, or yield of the reaction. If every copy of a target DNA is utilized as a template in every cycle of a polymerase chain reaction, then the mean efficiency is 100%. If 20 cycles of PCR are performed, then the yield will be 220, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The detection of at least one sequence change according to various preferred embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis or Dideoxy fingerprinting (ddF).

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations. Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered. Recently, endonucleases encoded by group I introns have been discovered that might have greater than 12 base-pair specificity, but again, these are few in number.

Allele specific oligonucleotide (ASO): If the change is not in a recognition sequence, then allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations. The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes and gsp/gip oncogenes. Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE. Attaching a GC clamp to one primer is critical to ensure that the amplified sequence has a low dissociation temperature. Modifications of the technique have been developed, using temperature gradients, and the method can be also applied to RNA:RNA duplexes.

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGEi). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient. TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations.

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations. The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

According to a presently preferred embodiment of the present invention the step of searching for any of the nucleic acid sequences described here, in tumor cells or in cells derived from a cancer patient is effected by any suitable technique, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

Detection may also optionally be performed with a chip or other such device. The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station. describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for a disease and/or pathological condition both rapidly and easily.

### Amino Acid Sequences and Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The present invention also encompasses polypeptides encoded by the polynucleotide sequences of the present invention, as well as polypeptides according to the amino acid sequences described herein. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth below, as can be determined using BlastP software of the National Center of Biotechnology Information (NCBI) using default parameters, optionally and preferably including the following: filtering on (this option filters repetitive or low-complexity sequences from the query using the Seg (protein) program), scoring matrix is BLOSUM62 for proteins, word size is 3, E value is 10, gap costs are 11, 1 (initialization and extension), and number of alignments shown is 50. Optionally and preferably, nucleic acid sequence homology/identity may be determined by using BlastN software of the National Center of Biotechnology Information (NCBI) using default parameters, which preferably include using the DUST filter program, and also preferably include having an E value of 10, filtering low complexity sequences and a word size of 11. Finally, the present invention also encompasses fragments of the above described polypeptides and polypeptides having mutations, such as deletions, insertions or substitutions of one or more amino acids, either naturally occurring or artificially induced, either randomly or in a targeted fashion.

It will be appreciated that peptides identified according the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

TABLE A
Table A: non-conventional or modified amino acids
which can be used with the present invention.

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| Carboxylate | | L-N-methylaspartic acid | Nmasp |
| aminoisobutyric acid | Aib | L-N-methylcysteine | Nmcys |
| aminonorbomyl- | Norb | L-N-methylglutamine | Nmgin |
| Carboxylate | | L-N-methylglutamic acid | Nmglu |
| Cyclohexylalanine | Chexa | L-N-methylhistidine | Nmhis |
| Cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

Since the peptides of the present invention are preferably utilized in diagnostics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclicization does not severely interfere with peptide characteristics, cyclic forms of the peptide can also be utilized.

The peptides of present invention can be biochemically synthesized such as by using standard solid phase techniques. These methods include exclusive solid phase synthesis well known in the art, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Synthetic peptides can be purified by preparative high performance liquid chromatography and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463 and also as described above.

### Antibodies

“Antibody” refers to a polypeptide ligand that is preferably substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)2, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′) or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Preferably, the antibody of this aspect of the present invention specifically binds at least one epitope of the polypeptide variants of the present invention. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

Optionally, a unique epitope may be created in a variant due to a change in one or more post-translational modifications, including but not limited to glycosylation and/or phosphorylation, as described below. Such a change may also cause a new epitope to be created, for example through removal of glycosylation at a particular site.

An epitope according to the present invention may also optionally comprise part or all of a unique sequence portion of a variant according to the present invention in combination with at least one other portion of the variant which is not contiguous to the unique sequence portion in the linear polypeptide itself, yet which are able to form an epitope in combination. One or more unique sequence portions may optionally combine with one or more other non-contiguous portions of the variant (including a portion which may have high homology to a portion of the known protein) to form an epitope.

### Immunoassays

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

To prepare an antibody that specifically binds to a marker, purified protein markers can be used. Antibodies that specifically bind to a protein marker can be prepared using any suitable methods known in the art.

After the antibody is provided, a marker can be detected and/or quantified using any of a number of well recognized immunological binding assays. Useful assays include, for example, an enzyme immune assay (EIA) such as enzyme-linked immunosorbent assay (ELISA), a radioimmune assay (RIA), a Western blot assay, or a slot blot assay see, e.g., U.S. Pat. Nos. 4,366,241; 4,376,110; 4,517,288; and 4,837,168). Generally, a sample obtained from a subject can be contacted with the antibody that specifically binds the marker.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead. Antibodies can also be attached to a solid support.

After incubating the sample with antibodies, the mixture is washed and the antibody-marker complex formed can be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal.

Preferably used are antibodies which specifically interact with the polypeptides of the present invention and not with wild type proteins or other isoforms thereof, for example. Such antibodies are directed, for example, to the unique sequence portions of the polypeptide variants of the present invention, including but not limited to bridges, heads, tails and insertions described in greater detail below. Preferred embodiments of antibodies according to the present invention are described in greater detail with regard to the section entitled “Antibodies”.

Radio-immunoassay (RIA): In one version, this method involves precipitation of the desired substrate and in the methods detailed hereinbelow, with a specific antibody and radiolabelled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a calorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a colorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

### Radio-Imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference as if fully set forth herein.

### Display Libraries

According to still another aspect of the present invention there is provided a display library comprising a plurality of display vehicles (such as phages, viruses or bacteria) each displaying at least 6, at least 7, at least 8, at least 9, at least 10, 10-15, 12-17, 15-20, 15-30 or 20-50 consecutive amino acids derived from the polypeptide sequences of the present invention.

Methods of constructing such display libraries are well known in the art. Such methods are described in, for example, Young A C, et al., “The three-dimensional structures of a polysaccharide binding antibody to Cryptococcus neoformans and its complex with a peptide from a phage display library: implications for the identification of peptide mimotopes” J Mol Biol 1997 Dec. 12; 274(4):622-34; Giebel L B et al. “Screening of cyclic peptide phage libraries identifies ligands that bind streptavidin with high affinities” Biochemistry 1995 Nov. 28; 34(47):15430-5; Davies E L et al., “Selection of specific phage-display antibodies using libraries derived from chicken immunoglobulin genes” J Immunol Methods 1995 Oct. 12; 186(1):125-35; Jones C R T al. “Current trends in molecular recognition and bioseparation” J Chromatogr A 1995 Jul. 14; 707(1):3-22; Deng S J et al. “Basis for selection of improved carbohydrate-binding single-chain antibodies from synthetic gene libraries” Proc Natl Acad Sci USA 1995 May 23; 92(11):4992-6; and Deng S J et al. “Selection of antibody single-chain variable fragments with improved carbohydrate binding by phage display” J Biol Chem 1994 Apr. 1; 269(13):9533-8, which are incorporated herein by reference.

The following sections relate to Candidate Marker Examples (first section) and to Experimental Data for these Marker Examples (second section).

It should be noted that Table numbering is restarted within each section.

### Candidate Marker Examples Section

This Section relates to Examples of sequences according to the present invention, including illustrative methods of selection thereof.

Description of the Methodology Undertaken to Uncover the Biomolecular Sequences of the Present Invention

Human ESTs and cDNAs were obtained from GenBank versions 136 (Jun. 15, 2003 ftp.ncbi.nih.gov/genbank/release.notes/gb136.release.notes); NCBI genome assembly of April 2003; RefSeq sequences from June 2003; Genbank version 139 (December 2003); Human Genome from NCBI (Build 34) (from October 2003); and RefSeq sequences from December 2003; and from the LifeSeq library of Incyte Corporation (Wilmington, Del., USA; ESTs only). With regard to GenBank sequences, the human EST sequences from the EST (GBEST) section and the human mRNA sequences from the primate (GBPRI) section were used; also the human nucleotide RefSeq mRNA sequences were used (see for example www.ncbi.nlm.nih.gov/Genbank/GenbankOverview.html and for a reference to the EST section, see www.ncbi.nlm.nih.gov/dbEST/; a general reference to dbEST, the EST database in GenBank, may be found in Boguski et al, Nat. Genet. 1993 August; 4(4):332-3; all of which are hereby incorporated by reference as if fully set forth herein).

Novel splice variants were predicted using the LEADS clustering and assembly system as described in Sorek, R., Ast, G. & Graur, D. Alu-containing exons are alternatively spliced. Genome Res 12, 1060-7 (2002); U.S. Pat. No. 6,625,545; and U.S. patent application Ser. No. 10/426,002, published as US20040101876 on May 27, 2004; all of which are hereby incorporated by reference as if fully set forth herein. Briefly, the software cleans the expressed sequences from repeats, vectors and immunoglobulins. It then aligns the expressed sequences to the genome taking alternatively splicing into account and clusters overlapping expressed sequences into “clusters” that represent genes or partial genes.

These were annotated using the GeneCarta (Compugen, Tel-Aviv, Israel) platform. The GeneCarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

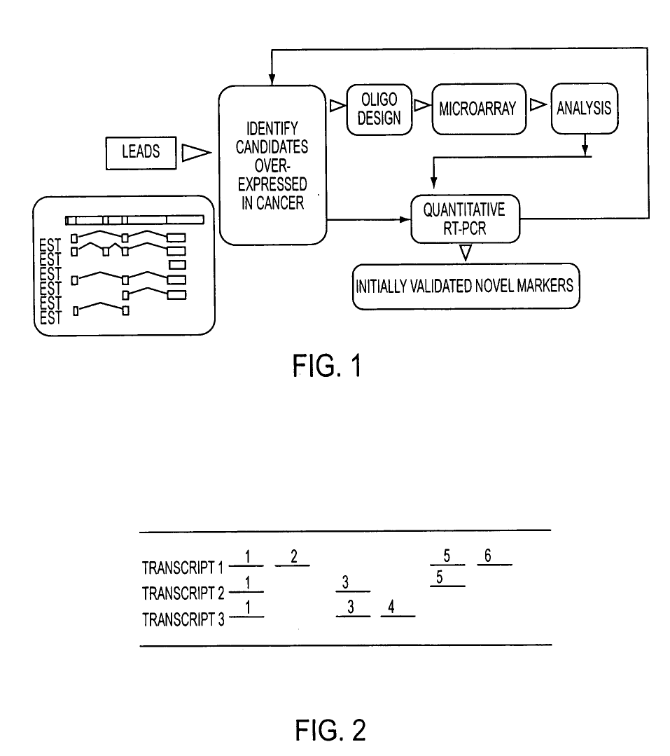

A brief explanation is provided with regard to the method of selecting the candidates. However, it should noted that this explanation is provided for descriptive purposes only, and is not intended to be limiting in any way. The potential markers were identified by a computational process that was designed to find genes and/or their splice variants that are over-expressed in tumor tissues, by using databases of expressed sequences. Various parameters related to the information in the EST libraries, determined according to a manual classification process, were used to assist in locating genes and/or splice variants thereof that are over-expressed in cancerous tissues. The detailed description of the selection method is presented in Example 1 below. The cancer biomarkers selection engine and the following wet validation stages are schematically summarized in FIG. 1.

### Example 1

### Identification of Differentially Expressed Gene Products

### Algorithm

In order to distinguish between differentially expressed gene products and constitutively expressed genes (i.e., house keeping genes ) an algorithm based on an analysis of frequencies was configured. A specific algorithm for identification of transcripts over expressed in cancer is described hereinbelow.

Dry Analysis

Library annotation—EST libraries are manually classified according to:

(i) Tissue origin

(ii) Biological source—Examples of frequently used biological sources for construction of EST libraries include cancer cell-lines; normal tissues; cancer tissues; fetal tissues; and others such as normal cell lines and pools of normal cell-lines, cancer cell-lines and combinations thereof. A specific description of abbreviations used below with regard to these tissues/cell lines etc is given above.

(iii) Protocol of library construction—various methods are known in the art for library construction including normalized library construction; non-normalized library construction; subtracted libraries; ORESTES and others. It will be appreciated that at times the protocol of library construction is not indicated.

The following rules are followed:

EST libraries originating from identical biological samples are considered as a single library.

EST libraries which included above-average levels of contamination, such as DNA contamination for example, were eliminated. The presence of such contamination was determined as follows. For each library, the number of unspliced ESTs that are not fully contained within other spliced sequences was counted. If the percentage of such sequences (as compared to all other sequences) was at least 4 standard deviations above the average for all libraries being analyzed, this library was tagged as being contaminated and was eliminated from further consideration in the below analysis (see also Sorek, R. & Safer, H. M. A novel algorithm for computational identification of contaminated EST libraries. Nucleic Acids Res 31, 1067-74 (2003) for further details).

Clusters (genes) having at least five sequences including at least two sequences from the tissue of interest were analyzed. Splice variants were identified by using the LEADS software package as described above.

### Example 2

### Identification of Genes Over Expressed in Cancer

Two different scoring algorithms were developed.

Libraries score—candidate sequences which are supported by a number of cancer libraries, are more likely to serve as specific and effective diagnostic markers.

The basic algorithm—for each cluster the number of cancer and normal libraries contributing sequences to the cluster was counted. Fisher exact test was used to check if cancer libraries are significantly over-represented in the cluster as compared to the total number of cancer and normal libraries.

Library counting: Small libraries (e.g., less than 1000 sequences) were excluded from consideration unless they participate in the cluster. For this reason, the total number of libraries is actually adjusted for each cluster.

Clones no. score—Generally, when the number of ESTs is much higher in the cancer libraries relative to the normal libraries it might indicate actual over-expression.

The Algorithm—

Clone counting: For counting EST clones each library protocol class was given a weight based on our belief of how much the protocol reflects actual expression levels:

(i) non-normalized: 1

(ii) normalized: 0.2

(iii) all other classes: 0.1

Clones number score—The total weighted number of EST clones from cancer libraries was compared to the EST clones from normal libraries. To avoid cases where one library contributes to the majority of the score, the contribution of the library that gives most clones for a given cluster was limited to 2 clones.

The score was computed as

$$\frac{\frac{c + 1}{C}}{\frac{n + 1}{N}}$$

where:

c—weighted number of “cancer” clones in the cluster.

C—weighted number of clones in all “cancer” libraries.

n—weighted number of “normal” clones in the cluster.

N—weighted number of clones in all “normal” libraries.

Clones number score significance—Fisher exact test was used to check if EST clones from cancer libraries are significantly over-represented in the cluster as compared to the total number of EST clones from cancer and normal libraries.

Two search approaches were used to find either general cancer-specific candidates or tumor specific candidates.
Libraries/sequences originating from tumor tissues are counted as well as libraries originating from cancer cell-lines (“normal” cell-lines were ignored).
Only libraries/sequences originating from tumor tissues are counted

### Example 3

### Identification of Tissue Specific Genes

For detection of tissue specific clusters, tissue libraries/sequences were compared to the total number of libraries/sequences in cluster. Similar statistical tools to those described in above were employed to identify tissue specific genes. Tissue abbreviations are the same as for cancerous tissues, but are indicated with the header “normal tissue”.

The algorithm—for each tested tissue T and for each tested cluster the following were examined:

1. Each cluster includes at least 2 libraries from the tissue T. At least 3 clones (weighed—as described above) from tissue T in the cluster; and

2. Clones from the tissue T are at least 40% from all the clones participating in the tested cluster

Fisher exact test P-values were computed both for library and weighted clone counts to check that the counts are statistically significant.

### Example 4

### Identification of Splice Variants Over Expressed in Cancer of Clusters which are not Over Expressed in Cancer

Cancer-specific splice variants containing a unique region were identified.

Identification of unique sequence regions in splice variants

A Region is defined as a group of adjacent exons that always appear or do not appear together in each splice variant.

A “segment” (sometimes referred also as “seg” or “node”) is defined as the shortest contiguous transcribed region without known splicing inside.

Only reliable ESTs were considered for region and segment analysis. An EST was defined as unreliable if:

(i) Unspliced;

(ii) Not covered by RNA;

(iii) Not covered by spliced ESTs; and

(iv) Alignment to the genome ends in proximity of long poly-A stretch or starts in proximity of long poly-T stretch.

Only reliable regions were selected for further scoring. Unique sequence regions were considered reliable if:

(i) Aligned to the genome; and

(ii) Regions supported by more than 2 ESTs.

The Algorithm

Each unique sequence region divides the set of transcripts into 2 groups:

| | |
|---|---|
| (i) Transcripts containing this region | (group TA). |
| (ii) Transcripts not containing this region | (group TB). |

The set of EST clones of every cluster is divided into 3 groups:

(i) Supporting (originating from) transcripts of group TA (S1).

(ii) Supporting transcripts of group TB (S2).

(iii) Supporting transcripts from both groups (S3).

Library and clones number scores described above were given to S1 group.

Fisher Exact Test P-values were used to check if:

S1 is significantly enriched by cancer EST clones compared to S2; and

S1 is significantly enriched by cancer EST clones compared to cluster background (S1+S2+S3).

Identification of unique sequence regions and division of the group of transcripts accordingly is illustrated in FIG. 2. Each of these unique sequence regions corresponds to a segment, also termed herein a “node”. Region 1: common to all transcripts, thus it is not considered; Region 2: specific to Transcript 1: T_{—}1 unique regions (2+6) against T_{—}2+3 unique regions (3+4); Region 3: specific to Transcripts 2+3: T_{—}2+3 unique regions (3+4) against T1 unique regions (2+6); Region 4: specific to Transcript 3: T_{—}3 unique regions (4) against T1+2 unique regions (2+5+6); Region 5: specific to Transcript 1+2: T_{—}1+2 unique regions (2+5+6) against T3 unique regions (4); Region 6: specific to Transcript 1: same as region 2.

### Example 5

### Identification of Cancer Specific Splice Variants of Genes Over Expressed in Cancer

A search for EST supported (no mRNA) regions for genes of:

(i) known cancer markers

(ii) Genes shown to be over-expressed in cancer in published micro-array experiments.

Reliable EST supported-regions were defined as supported by minimum of one of the following:

(i) 3 spliced ESTs; or

(ii) 2 spliced ESTs from 2 libraries;

(iii) 10 unspliced ESTs from 2 libraries, or

(iv) 3 libraries.

### Actual Marker Examples

The following examples relate to specific actual marker examples.

### Experimental Examples Section

This Section relates to Examples describing experiments involving these sequences, and illustrative, non-limiting examples of methods, assays and uses thereof. The materials and experimental procedures are explained first, as all experiments used them as a basis for the work that was performed.

The markers of the present invention were tested with regard to their expression in various cancerous and non-cancerous tissue samples. A description of the samples used in the panel is provided in Tables 1 and 1_{—}1 below. A description of the samples used in the normal tissue panel is provided in Tables 2 and 2_{—}2 below. Tests were then performed as described in the “Materials and Experimental Procedures” section below. The key for the is listed in tables 1_{—}1_{—}1.

TABLE 1
Tissue samples in testing panel

| COLON PANEL | | | | | | |
|---|---|---|---|---|---|---|
| sample name | Lot No. | tissue | source | pathology | Grade | gender/age |
| 58-B-Adeno G1 | A609152 | Colon | biochain | Adenocarcinoma | 1 | M/73 |
| 59-B-Adeno G1 | A609059 | Colon | biochain | Adenocarcinoma, Ulcer | 1 | M/58 |
| 14-CG-Polypoid Adeno G1 D-C | CG-222 (2) | Rectum | Ichilov | Well polypoid adeocarcinoma Duke's C | | F/49 |
| 17-CG-Adeno G1-2 | CG-163 | Rectum | Ichilov | Adenocarcinoma | 2 | M/73 |
| 10-CG-Adeno G1-2 D-B2 | CG-311 | Sigmod co [symbol] | Ichilov | Adenocarcinoma Astler-Coller B2. | 1-2 | M/88 |
| 11-CG-Adeno G1-2 D-C2 | CG-337 | Colon | Ichilov | Adenocarcinoma Astler-Coller C2. | 1-2 | NA |
| 6-CG-Adeno G1-2 D-C2 | CG-303 (3) | Colon | Ichilov | Adenocarcinoma Astler-Coller C2. | 1-2 | F/77 |
| 5-CG-Adeno G2 | CG-308 | Colon Sign [symbol] | Ichilov | Adenocarcinoma. | 2 | F/80 |
| 16-CG-Adeno G2 | CG-278C | colon | Ichilov | Adenocarcinoma | 2 | F/60 |
| 56-B-Adeno G2 | A609148 | Colon | biochain | Adenocarcinoma | 2 | F48 |
| 61-B-Adeno G2 | A606258 | Colon | biochain | Adenocarcinoma, Ulcer | 2 | M/41 |
| 60-B-Adeno G2 | A609058 | Colon | biochain | Adenocarcinoma, Ulcer | 2 | M/67 |
| 22-CG-Adeno G2 D-B | CG-229C | Colon | Ichilov | Adenocarcinoma Duke's B | 2 | F/55 |
| 1-CG-Adeno G2 D-B2 | CG-335 | Cecum | Ichilov | Adenocarcinoma Dukes B2. | 2 | F/66 |
| 12-CG-Adeno G2 D-B2 | CG-340 | Colon Sign [symbol] | Ichilov | Adenocarcinoma Astler-Coller B2. | 2 | M/66 |
| 28-CG-Adeno G2 D-B2 | CG-284 | sigma | Ichilov | Adenocarcinoma Duke's B2 | 2 | F/72 |
| 2-CG-Adeno G2 D-C2 | CG-307 X2 | Cecum | Ichilov | Adenocarcinoma Astler-Coller C2. | 2 | F/89 |
| 9-CG-Adeno G2 D-D | CG-297 X2 | Rectum | Ichilov | Adenocarcinoma Dukes D. | 2 | M/62 |
| 13-CG-Adeno G2 D-D | CG-290 X2 | Rectosigm [symbol] | Ichilov | Adenocarcinoma Dukes D. | 2 | M/47 |
| 26-CG-Adeno G2 D-D | CG-283 | sigma | Ichilov | Colonic adenocarcinoma Duke's D | 2 | F/63 |
| 4-CG-Adeno G3 | CG-276 | Colon | Ichilov | Carcinoma. | 3 | M/64 |
| 53-B-Adeno G3 | A609161 | Colon | biochain | Adenocarcinoma | 3 | F/53 |
| 54-B-Adeno G3 | A609142 | Colon | biochain | Adenocarcinoma | 3 | M/53 |
| 55-B-Adeno G3 | A609144 | Colon | biochain | Adenocarcinoma | 3 | M/68 |
| 57-B-Adeno G3 | A609150 | Colon | biochain | Adenocarcinoma | 3 | F/45 |
| 72-CG-Adeno G3 | CG-309 | colon | Ichilov | Adenocarcinoma | 3 | F/88 |
| 20-CG-Adeno G3 D-B2 | CG-249 | Colon | Ichilov | Ulcerated adenocarcinoma Duke's B2 | 3 | M/36 |
| 7-CG-Adeno D-A | CG-235 | Rectum | Ichilov | Adenocarcinoma intramucosal Duke's A. | | F/66 |
| 23-CG-Adeno D-C | CG-282 | sigma | Ichilov | Mucinus adenocarcinoma Astler Coller C | | M/51 |
| 3-CG-Muc adeno D-D | CG-224 | Colon | Ichilov | Mucinois adenocarcinoma Duke's D | | M/48 |
| 18-CG-Adeno | CG-22C | Colon | Ichilov | Adenocarcinoma | | NA |
| 19-CG-Adeno | CG-19C (1) | Colon | Ichilov | Adenocarcinoma | | NA |
| 21-CG-Adeno | CG-18C | Colon | Ichilov | Adenocarcinoma | | NA |
| 24-CG-Adeno | CG-12 (2) | Colon | Ichilov | Adenocarcinoma | | NA |
| 25-CG-Adeno | CG-2 | Colon | Ichilov | Adenocarcinoma | | NA |
| 27-CG-Adeno | CG-4 | Colon | Ichilov | Adenocarcinoma | | NA |
| 8-CG-diverticolosis, diverticulitis | CG-291 | Wall of sig [symbol] | Ichilov | Diverticolosis and diverticulitis of the Colon | | F/65 |
| 46-CG-Crohn's disease | CG-338C | Cecum | Ichilov | Crohn's disease | | M/22 |
| 47-CG-Crohn's disease | CG-338AC | Colon | Ichilov | Crohn's disease. | | M/22 |
| 42-CG-N M20 | CG-249N | Colon | Ichilov | Normal | | M/36 |
| 43-CG-N M8 | CG-291N | Wall of sig [symbol] | Ichilov | Normal | | F/65 |
| 44-CG-N M21 | CG-18N | Colon | Ichilov | Normal | | NA |
| 45-CG-N M11 | CG-337N | Colon | Ichilov | Normal | | M/75 |
| 49-CG-N M14 | CG-222N | Rectum | Ichilov | Normal | | F/49 |
| 50-CG-N M5 | CG-308N | Sigma | Ichilov | Within normal limits | | F/80 |
| 51-CG-N M26 | CG-283N | Sigma | Ichilov | Normal | | F/63 |
| 41-B-N | A501156 | Colon | biochain | Normal PM | | M/78 |
| 52-CG-N | CG-309TR | Colon | Ichilov | Within normal limits | | F/88 |
| 62-B-N | A608273 | Colon | biochain | Normal PM | | M/66 |
| 63-B-N | A609260 | Colon | biochain | Normal PM | | M/61 |
| 64-B-N | A609261 | Colon | biochain | Normal PM | | F/68 |
| 65-B-N | A607115 | Colon | biochain | Normal PM | | M/24 |
| 66-B-N | A609262 | Colon | biochain | Normal PM | | M/58 |
| 67-B-N | A406029 | Colon | biochain | Normal PM (Pool of 10) | | |
| 69-B-N | A411078 | Colon | biochain | Normal PM (Pool of 10) | | F&M |
| 70-Cl-N | 1110101 | Colon | clontech | Normal PM (Pool of 3) | | |
| 71-Am-N | 071P10B | Colon | Ambion | Normal (IC BLEED) | | F/34 |
| 15-CG-Adeno D-A | CG-235 | Rectum | Ichilov | Adenocarcinoma intramucosal Duke's A. | | F/66 |

[symbol]

indicates data missing or illegible when filed

TABLE 1_1
Colon cancer testing panel

| | | | sample_id | | | | | | | |
|---|---|---|---|---|---|---|---|---|---|---|
| | | | (GCI)/ | | | | | | | |
| | | | case id | | | | | | | |
| | | | (Asterand)/ | TISSUE ID | | | | | | |
| | | | lot | (GCI)/ | | | | | | |
| | | | no. | specimen | Sample | | | | | |
| | Source/ | sample | (old | ID | ID | | Diag | Specimen | | |
| Tissue | Delivery | name | samples | (Asterand) | (Asterand) | Diag | remarks | location | Gr | TNM |
| CC | Asterand | 1-As- | 18036 | 31312 | 31312B1 | Aden | | Cec | 3 | TXN0M0 |
| | | AdenS0 | | | | | | | | |
| CC | GCI | 2-GC- | 4QDH8 | 4QDH8ADT | | Aden | | Dis C | | |
| | | Adeno | | | | | | | | |
| | | SI | | | | | | | | |
| CC | Ichilov | 3-(7)- | CG-235 | | | Al | | Rectum | UN | |
| | | Ic- | | | | | | | | |
| | | Adeno | | | | | | | | |
| | | SI | | | | | | | | |
| CC | GCI | 4-GC- | NTAI8 | NTAI8AOU | | Aden | | Cec | | |
| | | Adeno | | | | | | | | |
| | | SI | | | | | | | | |
| CC | GCI | 5-GC- | ARA7P | ARA7PAQA | | Aden | | Ret, | | |
| | | Adeno | | | | | | Low | | |
| | | SI | | | | | | Ant | | |
| CC | Ichilov | 6-(20)- | CG-249 | | | UA | | | 3 | |
| | | Ic- | | | | | | | | |
| | | Adeno | | | | | | | | |
| | | SIIA | | | | | | | | |
| CC | GCI | 7-GC- | AFTS6 | AFTS6AP6 | | Aden | | | | |
| | | Adeno | | | | | | | | |
| | | SIIA | | | | | | | | |
| CC | GCI | 8-GC- | 5CYDK | 5CYDKACS | | Aden | | | | |
| | | Adeno | | | | | | | | |
| | | SIIA | | | | | | | | |
| CC | GCI | 9-GC- | XKSLS | XKSLSAF7 | | Aden | | | | |
| | | Adeno | | | | | | | | |
| | | SIIA | | | | | | | | |
| CC | GCI | 10- | B4RU8 | B4RU8A8Q | | Aden | | | | |
| | | GC- | | | | | | | | |
| | | Adeno | | | | | | | | |
| | | SIIA | | | | | | | | |
| CC | GCI | 11- | HB8EY | HB8EYA8I | | Aden | | | | |
| | | GC- | | | | | | | | |
| | | Adeno | | | | | | | | |
| | | SIIA | | | | | | | | |
| CC | Ichilov | 12- | CG-229C | | | Aden | | | 2 | |
| | | (22)- | | | | | | | | |
| | | Adeno | | | | | | | | |
| | | SII | | | | | | | | |
| CC | GCI | 13- | X8C7X | X8C7XATL | | Aden | | | | |
| | | GC- | | | | | | | | |
| | | Adeno | | | | | | | | |
| | | SIIA | | | | | | | | |
| CC | GCI | 14- | HCP6K | HCP6KA8Z | | Aden | | | | |
| | | GC- | | | | | | | | |
| | | Adeno | | | | | | | | |
| | | SIIA | | | | | | | | |
| CC | GCI | 15- | ZX4X7 | ZX4X7AXA | | Aden | | | | |
| | | GC- | | | | | | | | |
| | | Adeno | | | | | | | | |
| | | SIIA | | | | | | | | |
| CC | Asterand | 16-As- | 17915 | 31176 | 31176A1 | Aden | | | 2-3 | T3N0M0 |
| | | Adeno | | | | | | | | |
| | | SIIA | | | | | | | | |
| CC | Ichilov | 17-(1)- | CG-335 | | | Aden | | Cec | 2 | |
| | | Ic- | | | | | | | | |
| | | Adeno | | | | | | | | |
| | | SIIA | | | | | | | | |
| CC | Asterand | 19-As- | 12772 | 18885 | 18885A1 | Aden | | rectum | 2 | T3NXM0 |
| | | Adeno | | | | | | | | |
| | | SIIA | | | | | | | | |
| CC | GCI | 20- | JFYXP | JFYXPAMP | | Aden | | | | |
| | | GC- | | | | | | | | |
| | | Adeno | | | | | | | | |
| | | SIIA | | | | | | | | |
| CC | GCI | 21- | OJXW9 | OJXW9ASR | | Aden | | | | |
| | | GC- | | | | | | | | |
| | | Adeno | | | | | | | | |
| | | SIIA | | | | | | | | |
| CC | Ichilov | 22- | CG-284 | | | Aden | | sigma | 2 | |
| | | (28)- | | | | | | | | |
| | | Ic- | | | | | | | | |
| | | Adeno | | | | | | | | |
| | | SIIA | | | | | | | | |
| CC | Ichilov | 23- | CG-311 | | | Aden | | Sig | 1-2 | |
| | | (10)- | | | | | | Col | | |
| | | Ic- | | | | | | | | |
| | | Adeno | | | | | | | | |
| | | SIIA | | | | | | | | |
| CC | Ichilov | 24- | CG-222 | | | WP | | Rectum | | |
| | | (14)- | (2) | | | Aden | | | | |
| | | Ic- | | | | | | | | |
| | | Adeno | | | | | | | | |
| | | SIII | | | | | | | | |
| CC | Ichilov | 25- | CG-282 | | | MA | | sigma | UN | |
| | | (23)- | | | | | | | | |
| | | Ic- | | | | | | | | |
| | | Adeno | | | | | | | | |
| | | SIII | | | | | | | | |
| CC | GCI | 26- | OTPI7 | OTPI7AWY | | Aden | | | | |
| | | GC- | | | | | | | | |
| | | Adeno | | | | | | | | |
| | | SIII | | | | | | | | |
| CC | GCI | 27- | IG9NK | IG9NKAD3 | | MA | | | | |
| | | GC- | | | | | | | | |
| | | Adeno | | | | | | | | |
| | | SIII | | | | | | | | |
| CC | GCI | 28- | 53OM7 | 53OM7AGL | | Aden | | | | |
| | | GC- | | | | | | | | |
| | | Adeno | | | | | | | | |
| | | SIII | | | | | | | | |
| CC | GCI | 29- | BLUW6 | BLUW6A6Y | | Aden | | | | |
| | | GC- | | | | | | | | |
| | | Adeno | | | | | | | | |
| | | SIII | | | | | | | | |
| CC | GCI | 30- | VZ6QA | VZ6QAAFA | | Aden | | RECTUM | | |
| | | GC- | | | | | | | | |
| | | Adeno | | | | | | | | |
| | | SIII | | | | | | | | |
| CC | Ichilov | 31-(6)- | CG-303 | | | Aden | | | 1-2 | |
| | | Ic- | (3) | | | | | | | |
| | | Adeno | | | | | | | | |
| | | SIII | | | | | | | | |
| CC | Ichilov | 32-(2)- | CG-307 | | | Aden | | Cecum | 2 | |
| | | Ic- | | | | | | | | |
| | | Adeno | | | | | | | | |
| | | SIII | | | | | | | | |
| CC | Ichilov | 33- | CG-337 | | | Aden | | | 1-2 | |
| | | (11)- | | | | | | | | |
| | | Ic- | | | | | | | | |
| | | Adeno | | | | | | | | |
| | | SIII | | | | | | | | |
| CC | Asterand | 34-As- | 18462 | 40971 | 40971A1 | TA | | Sig | 2 | TXN2M0 |
| | | Adeno | | | | | | Col | | |
| | | SIIIC | | | | | | | | |
| CC | Ichilov | 35- | CG-290 | | | Aden | | Rect | 2 | |
| | | (13)- | | | | | | Col | | |
| | | Ic- | | | | | | | | |
| | | Adeno | | | | | | | | |
| | | SIV | | | | | | | | |
| CC | GCI | 36- | 7D7QV | 7D7QVAE6 | | Aden | | | | |
| | | GC- | | | | | | | | |
| | | Adeno | | | | | | | | |
| | | SIV | | | | | | | | |
| CC | GCI | 37- | 38U4V | 38U4VAA4 | | Aden | | | | |
| | | GC- | | | | | | | | |
| | | Adeno | | | | | | | | |
| | | SIV | | | | | | | | |
| CC | Ichilov | 38-(9)- | CG-297 | | | Aden | | Rectum | 2 | |
| | | Ic- | | | | | | | | |
| | | Adeno | | | | | | | | |
| | | SIV | | | | | | | | |
| CC | Ichilov | 71- | CG-278C | | | Aden | | | 2 | |
| | | (16)- | | | | | | | | |
| | | Ic- | | | | | | | | |
| | | Adeno | | | | | | | | |
| CC | Ichilov | 72-(4)- | CG-276 | | | Carc | | | 3 | |
| | | Ic- | | | | | | | | |
| | | Adeno | | | | | | | | |
| CC | Ichilov | 73- | CG-163 | | | Aden | | Rectum | 2 | |
| | | (17)- | | | | | | | | |
| | | Ic- | | | | | | | | |
| | | Adeno | | | | | | | | |
| CC | Ichilov | 74-(5)- | CG-308 | | | Aden | | Col | 2 | |
| | | Ic- | | | | | | Sig | | |
| | | Adeno | | | | | | | | |
| CC | Ichilov | 75- | CG-309 | | | Aden | | | 3 | |
| | | (72)- | | | | | | | | |
| | | Ic- | | | | | | | | |
| | | Adeno | | | | | | | | |
| CC | Ichilov | 76- | CG-22C | | | Aden | | | UN | |
| | | (18)- | | | | | | | | |
| | | Ic- | | | | | | | | |
| | | Adeno | | | | | | | | |
| CC | Ichilov | 78- | CG-18C | | | Aden | | | UN | |
| | | (21)- | | | | | | | | |
| | | Ic- | | | | | | | | |
| | | Adeno | | | | | | | | |
| CC | Ichilov | 79- | CG-12 | | | Aden | | | UN | |
| | | (24)- | | | | | | | | |
| | | Ic- | | | | | | | | |
| | | Adeno | | | | | | | | |
| CC | Ichilov | 80- | CG-2 | | | Aden | | | UN | |
| | | (25)- | | | | | | | | |
| | | Ic- | | | | | | | | |
| | | Adeno | | | | | | | | |
| CC | biochain | 82- | A606258 | | | Aden, | | | 2 | |
| | | (61)- | | | | Ulcer | | | | |
| | | Bc- | | | | | | | | |
| | | Adeno | | | | | | | | |
| CC | biochain | 83- | A609150 | | | Aden | | | 3 | |
| | | (57)- | | | | | | | | |
| | | Bc- | | | | | | | | |
| | | Adeno | | | | | | | | |
| CC | biochain | 84- | A609148 | | | Aden | | | 2 | |
| | | (56)- | | | | | | | | |
| | | Bc- | | | | | | | | |
| | | Adeno | | | | | | | | |
| CC | biochain | 85- | A609161 | | | Aden | | | 3 | |
| | | (53)- | | | | | | | | |
| | | Bc- | | | | | | | | |
| | | Adeno | | | | | | | | |
| CC | biochain | 86- | A609142 | | | Aden | | | 3 | |
| | | (54)- | | | | | | | | |
| | | Bc- | | | | | | | | |
| | | Adeno | | | | | | | | |
| CC | biochain | 87- | A609059 | | | Aden, | | | 1 | |
| | | (59)- | | | | Ulcer | | | | |
| | | Bc- | | | | | | | | |
| | | Adeno | | | | | | | | |
| CC | biochain | 88- | A609058 | | | Aden, | | | 2 | |
| | | (60)- | | | | Ulcer | | | | |
| | | Bc- | | | | | | | | |
| | | Adeno | | | | | | | | |
| CC | biochain | 89- | A609144 | | | Aden | | | 3 | |
| | | (55)- | | | | | | | | |
| | | Bc- | | | | | | | | |
| | | Adeno | | | | | | | | |
| CC | biochain | 90- | A609152 | | | Aden | | | 1 | |
| | | (58)- | | | | | | | | |
| | | Bc- | | | | | | | | |
| | | Adeno | | | | | | | | |
| CB | GCI | 40- | IG3OY | IG3OYN7S | | TS | | RT Col | | |
| | | GC- | | | | Aden | | | | |
| | | Ben | | | | | | | | |
| CB | GCI | 41- | GKIEY | GKIEYAV4 | | TS | | Prox T | | |
| | | GC- | | | | Aden | | Col | | |
| | | Ben | | | | HGD | | | | |
| CN | GCI | 42- | AGVTC | AGVTCNK7 | | NC | DIV | | | |
| | | GC-N | | | | | | | | |
| | | PS | | | | | | | | |
| CN | Asterand | 43-As- | 8956 | 9153 | 9153B1 | NC | | | | |
| | | N PS | | | | | | | | |
| CN | GCI | 44- | IG3OY | IG3OYN7S | | NC | | RT Col | | |
| | | GC-N | | | | | | | | |
| | | PS | | | | | | | | |
| CN | GCI | 45- | K9OYX | K9OYXN4F | | NC | Divs | LT Col | | |
| | | GC-N | | | | | w/F DIV | | | |
| | | PS | | | | | | | | |
| CN | Asterand | 46-As- | 23024 | 74445 | 74445B1 | NC | Chr | | | |
| | | N PS | | | | | Divs | | | |
| CN | Asterand | 47-As- | 23049 | 71410 | 71410B2 | NC | Chr | | | |
| | | N PS | | | | | Divs | | | |
| CN | GCI | 48- | G7JJX | G7JJXAX7 | | NC | Divs | Sig | | |
| | | GC-N | | | | | w/ | Col | | |
| | | PS | | | | | DIV . . . | | | |
| CN | Asterand | 49-As- | 22900 | 74446 | 74446B1 | NC | AD | | | |
| | | N PS | | | | | w/AF | | | |
| CN | GCI | 50- | XVPZ2 | XVPZ2NDD | | NC | Div | | | |
| | | GC-N | | | | | | | | |
| | | PS | | | | | | | | |
| CN | GCI | 51- | CDSUV | CDSUVNR3 | | NC | CU | | | |
| | | GC-N | | | | | | | | |
| | | PS | | | | | | | | |
| CN | GCI | 52- | GP5KH | GP5KHAOC | | NC | Div | | | |
| | | GC-N | | | | | | | | |
| | | PS | | | | | | | | |
| CN | GCI | 53- | YUZNR | YUZNRNDN | | NC | Divs | Sig | | |
| | | GC-N | | | | | | Col | | |
| | | PS | | | | | | | | |
| CN | GCI | 54- | 28QN6 | 28QN6NI1 | | NC | TS | RT Col | | |
| | | GC-N | | | | | Aden | | | |
| | | PS | | | | | | | | |
| CN | GCI | 55- | GV6N8 | GV6N8NG9 | | NC | Divs, | | | |
| | | GC-N | | | | | PA | | | |
| | | PS | | | | | | | | |
| CN | GCI | 56- | ZJ17R | ZJ17RNIH | | NC | Tub | RT Col | | |
| | | GC-N | | | | | Aden | | | |
| | | PS | | | | | | | | |
| CN | GCI | 57- | 2EEBJ | 2EEBJN2Q | | NC | Div/Chr | | | |
| | | GC-N | | | | | Infl | | | |
| | | PS | | | | | | | | |
| CN | GCI | 58- | 68IX5 | 68IX5N1H | | NC | Chr Div | LT Col | | |
| | | GC-N | | | | | | | | |
| | | PS | | | | | | | | |
| CN | GCI | 59- | 9GEGL | 9GEGLN1V | | NC | Ext Divs | Sig | | |
| | | GC-N | | | | | | Col | | |
| | | PS | | | | | | | | |
| CN | GCI | 60- | PKU8O | PKU8OAJ3 | | NC | Divs, | Sig | | |
| | | GC-N | | | | | Chr | Col | | |
| | | PS | | | | | Div . . . | | | |
| CN | Asterand | 61-As- | 22903 | 74452 | 74452B1 | NC | MU | | | |
| | | N PS | | | | | w/MI | | | |
| CN | Asterand | 62-As- | 16364 | 31802 | 31802B1 | NC | UC | | | |
| | | N PS | | | | | | | | |
| CN | biochain | 63- | A607115 | | | N-PM | PM | | | |
| | | (65)- | | | | | | | | |
| | | Bc-N | | | | | | | | |
| | | PM | | | | | | | | |
| CN | Ambion | 64- | 071P10B | | | N-PM | PM | | | |
| | | (71)- | | | | | | | | |
| | | Am-N | | | | | | | | |
| | | PM | | | | | | | | |
| CN | biochain | 65- | A609262 | | | N-PM | PM | | | |
| | | (66)- | | | | | | | | |
| | | Bc-N | | | | | | | | |
| | | PM | | | | | | | | |
| CN | biochain | 66- | A609260 | | | N-PM | PM | | | |
| | | (63)- | | | | | | | | |
| | | Bc-N | | | | | | | | |
| | | PM | | | | | | | | |
| CN | biochain | 67- | A608273 | | | N-PM | PM | | | |
| | | (62)- | | | | | | | | |
| | | Bc-N | | | | | | | | |
| | | PM | | | | | | | | |
| CN | biochain | 68- | A609261 | | | N-PM | PM | | | |
| | | (64)- | | | | | | | | |
| | | Bc-N | | | | | | | | |
| | | PM | | | | | | | | |
| CN | biochain | 69- | A501156 | | | N-PM | PM | | | |
| | | (41)- | | | | | | | | |
| | | Bc-N | | | | | | | | |
| | | PM | | | | | | | | |
| CN | biochain | 70- | A406029 + | | | N-PM | PM | | | |
| | | (67)- | A411078 | | | P10 | | | | |
| | | Bc-N | | | | | | | | |
| | | PM | | | | | | | | |

| | | | | | | | | Dr. | | | |
|---|---|---|---|---|---|---|---|---|---|---|---|
| | | | | | | | Alcohol | per | Alc. | Recovery | Exc. |
| Tissue | CS | CS2 | Tumor % | Gender | age | Ethnic B | Status | day | Dur. | Type | Y. |
| CC | 0 | | 80 | F | 43 | CAU | NU | | | Auto | 2004 |
| CC | I | Duke A | 85 | F | 44 | WCAU | Y | 4 | | Surg | |
| CC | I | Duke A | | F | 66 | | | | | | |
| CC | I | Duke | 80 | M | 53 | WCAU | Y | — | | Surg | |
| | | B1 | | | | | | | | | |
| CC | I | Duke | 70 | F | 70 | WCAU | Y | 0 | | Surg | |
| | | B1 | | | | | | | | | |
| CC | IIA | Duke | | M | 36 | | | | | | |
| | | B2 | | | | | | | | | |
| CC | IIA | Duke | 75 | M | 39 | WCAU | N | 0 | | Surg | |
| | | B2 | | | | | | | | | |
| CC | IIA | Duke | 65 | M | 44 | WCAU | N | — | | Surg | |
| | | B2 | | | | | | | | | |
| CC | IIA | Duke | 65 | M | 48 | WCAU | Y | 10 | | Surg | |
| | | B2 | | | | | | | | | |
| CC | IIA | Duke | 65 | F | 50 | WCAU | N | — | | Surg | |
| | | B2 | | | | | | | | | |
| CC | IIA | Duke | 65 | M | 53 | WCAU | N | — | | Surg | |
| | | B2 | | | | | | | | | |
| CC | II | Duke B | | F | 55 | | | | | | |
| CC | IIA | Duke | 90 | M | 56 | WCAU | N | — | | Surg | |
| | | B2 | | | | | | | | | |
| CC | IIA | Duke | 80 | M | 58 | WCAU | Y | 4 | | Surg | |
| | | B2. | | | | | | | | | |
| CC | IIA | Duke | 90 | M | 60 | WCAU | Y | 5 | | Surg | |
| | | B2 | | | | | | | | | |
| CC | IIA | Duke | 60 | F | 64 | CAU | occ | 1 | 21-30 | Auto | 2004 |
| | | B2 | | | | | | drink/ | years | | |
| | | | | | | | | week | | | |
| CC | IIA | Duke | | F | 66 | | | | | | |
| | | B2 | | | | | | | | | |
| CC | IIA | Duke | 60 | F | 67 | CAU | NU | | | Surg | 2004 |
| | | B2 | | | | | | | | | |
| CC | IIA | Duke | 60 | F | 68 | WCAU | Y | — | | Surg | |
| | | B2 | | | | | | | | | |
| CC | IIA | Duke | 90 | F | 69 | WCAU | N | — | | Surg | |
| | | B2 | | | | | | | | | |
| CC | IIA | Duke | | F | 72 | | | | | | |
| | | B2 | | | | | | | | | |
| CC | e | Duke | | M | 88 | | | | | | |
| | IIA | B2 | | | | | | | | | |
| CC | III | Duke C | | F | 49 | | | | | | |
| CC | III | Duke C | | M | 51 | | | | | | |
| CC | III | Duke | 70 | F | 54 | WCAU | N | — | | Surg | |
| | | C2 | | | | | | | | | |
| CC | III | Duke | 90 | F | 54 | WCAU | N | — | | Surg | |
| | | C2 | | | | | | | | | |
| CC | III | Duke | 75 | F | 61 | WCAU | N | — | | Surg | |
| | | C2 | | | | | | | | | |
| CC | III | Duke | 85 | F | 64 | WCAU | N | — | | Surg | |
| | | C2 | | | | | | | | | |
| CC | III | Duke | 60 | M | 67 | WCAU | Y | 14 | | Surg | |
| | | C2 | | | | | | | | | |
| CC | III | Duke | | F | 77 | | | | | | |
| | | C2. | | | | | | | | | |
| CC | III | Duke | | F | 89 | | | | | | |
| | | C2. | | | | | | | | | |
| CC | III | Duke | | NA | NA | | | | | | |
| | | C2. | | | | | | | | | |
| CC | IIIC | | 76 | F | 68 | CAU | NU | | | Surg | 2005 |
| CC | IV | Duke | | M | 47 | | | | | | |
| | | D. | | | | | | | | | |
| CC | IV | Duke D | 80 | F | 52 | WCAU | Y | 3 | | Surg | |
| CC | IV | Duke D | 85 | F | 53 | WCAU | | | | Surg | |
| CC | IV | Duke | | M | 62 | | | | | | |
| | | D. | | | | | | | | | |
| CC | | UN | 50 | F | 60 | | | | | | |
| CC | | UN | 75 | M | 64 | | | | | | |
| CC | | UN | | M | 73 | | | | | | |
| CC | | UN | | F | 80 | | | | | | |
| CC | | UN | | F | 88 | | | | | | |
| CC | | UN | | NA | NA | | | | | | |
| CC | | UN | | NA | NA | | | | | | |
| CC | | UN | | NA | NA | | | | | | |
| CC | | UN | | NA | NA | | | | | | |
| CC | | UN | | M | 41 | | | | | | |
| CC | | UN | | F | 45 | | | | | | |
| CC | | UN | 40 | F | 48 | | | | | | |
| CC | | UN | | F | 53 | | | | | | |
| CC | | UN | | M | 53 | | | | | | |
| CC | | UN | | M | 58 | | | | | | |
| CC | | UN | | M | 67 | | | | | | |
| CC | | UN | | M | 68 | | | | | | |
| CC | | UN | | M | 73 | | | | | | |
| CB | | | | F | 48 | WCAU | Y | 1 | | Surg | |
| CB | | | | F | 75 | WCAU | N | — | | Surg | |
| CN | | | 0 | M | 45 | WCAU | N | — | | Surg | |
| CN | | | 0 | F | 46 | CAU | NU | | | Surg | 2002 |
| CN | | | 0 | F | 48 | WCAU | Y | 1 | | Surg | |
| CN | | | 0 | F | 50 | WCAU | N | — | | Surg | |
| CN | | | 0 | F | 52 | CAU | Occ | | | Surg | 2005 |
| CN | | | 0 | F | 52 | CAU | occ | | | Surg | 2005 |
| CN | | | 0 | M | 52 | WCAU | N | — | | Surg | |
| CN | | | 0 | M | 54 | CAU | Cur U | | | Surg | 2005 |
| CN | | | 0 | F | 55 | WCAU | N | — | | Surg | |
| CN | | | 0 | M | 55 | WCAU | N | — | | Surg | |
| CN | | | 0 | F | 57 | WCAU | Y | 6 | | Surg | |
| CN | | | 0 | F | 57 | WCAU | Y | 1 | | Surg | |
| CN | | | 0 | M | 59 | WCAU | Y | 42 | | Surg | |
| CN | | | 0 | F | 61 | WCAU | Y | 3 | | Surg | |
| CN | | | 0 | M | 61 | WCAU | Y | — | | Surg | |
| CN | | | 0 | F | 66 | WCAU | Y | 4 | | Surg | |
| CN | | | 0 | F | 66 | WCAU | N | — | | Surg | |
| CN | | | 0 | M | 68 | WCAU | N | — | | Surg | |
| CN | | | 0 | F | 69 | WCAU | N | — | | Surg | |
| CN | | | 0 | M | 71 | CAU | Occ | | | Surg | 2005 |
| CN | | | 0 | F | 74 | WCAU | Occ | | | Surg | 2004 |
| CN | | | | M | 24 | | | | | | |
| CN | | | | F | 34 | | | | | | |
| CN | | | | M | 58 | | | | | | |
| CN | | | | M | 61 | | | | | | |
| CN | | | | M | 66 | | | | | | |
| CN | | | | F | 68 | | | | | | |
| CN | | | | M | 78 | | | | | | |
| CN | | | | F&M | M | | | | | | |
| | | | | | (26-78) | | | | | | |
| | | | | | &F | | | | | | |
| | | | | | (53-77). | | | | | | |

TABLE 1_1_1

| Key | Full Name |
|---|---|
| CC | Colon Cancer |
| CB | Colon Benign |
| CN | Colon Normal |
| WT | Weight |
| HT | Height |
| Aden | Adenocarcinoma |
| AI | Adenocarcinoma intramucosal |
| UA | Ulcerated adenocarcinoma |
| WP Aden | Well polypoid adeocarcinoma |
| MA | Mucinus adenocarcinoma |
| TA | Tubular adenocarcinoma |
| Carc | Carcinoma |
| TS Aden | TUBULOVILLOUS ADENOMA |
| TS Aden HGD | TUBULOVILLOUS ADENOMA |
| | with HIGH GRADE DYSPLASIA |
| NC | Normal Colon |
| N-PM | Normal PM |
| N-PM P10 | Normal PM (Pool 10) |
| Diag | Diagnosis |
| Div | DIVERTICULITIS |
| Divs w/F DIV | Diverticulosis with Focal DIVERTICULITIS |
| Chr Divs | Chronic diverticulosis |
| Divs w/DIV . . . | DIVERTICULOSIS WITH DIVERTICULITIS |
| | AND FOCAL ABSCESS FORMATION; NO |
| | MALIGNANCY |
| AD w/AF | Acute diverticulitis with abscess formation |
| CU | CECAL ULCERATION |
| Divs, PA | DIVERTICULOSIS AND PERICOLIC ABSCESS |
| Tub Aden | TUBULAR ADENOMA |
| Div/Chr Infl | DIVERTICULOSIS/CHRONIC INFLAMMATION |
| Chr Div | CHRONIC DIVERTICULITIS |
| Ext Divs | EXTENSIVE DIVERTICULOSIS |
| Divs, Chr Div . . . | DIVERTICULOSIS AND |
| | CHRONIC DIVERTICULITIS, |
| | SEROSAL FIBROSIS AND CHRONIC |
| | SEROSITIS |
| MU w/MI | Mucosal ulceration with mural inflammation |
| UC | Ulcerative colitis |
| Cec | cecum |
| Dis C | DISTAL COLON |
| Ret, Low Ant | RETROSIGMOID, LOW ANTERIOR |
| Rect Col | Rectosigmoidal colon |
| Sig col | Sigmod colon |
| Col Sig | Colon Sigma |
| RT Col | RIGHT COLON |
| Prox T Col | PROXIMAL TRANSVERSE COLON |
| LT Col | Left Colon |
| Gr | Grade |
| CS | Cancer Stage |
| Ethnic B | Ethnic background |
| NU | Never Used |
| Occ | Occasion |
| Cur U | Current use |
| Dr. per day | Drinks per day |
| Alc. Dur. | Alcohol Duration |
| Auto. | Autopsy |
| Surg. | Surgical |
| Exc. Y. | Excision Year |

TABLE 2
Tissue samples in normal panel:

| | Lot no. | Source | Tissue | Pathology | Sex/Age |
|---|---|---|---|---|---|
| 1-Am-Colon (C71) | 071P10B | Ambion | Colon | PM | F/43 |
| 2-B-Colon (C69) | A411078 | Biochain | Colon | PM-Pool of 10 | M&F |
| 3-Cl-Colon (C70) | 1110101 | Clontech | Colon | PM-Pool of 3 | M&F |
| 4-Am-Small Intestine | 091P0201A | Ambion | Small Intestine | PM | M/75 |
| 5-B-Small Intestine | A501158 | Biochain | Small Intestine | PM | M/63 |
| 6-B-Rectum | A605138 | Biochain | Rectum | PM | M/25 |
| 7-B-Rectum | A610297 | Biochain | Rectum | PM | M/24 |
| 8-B-Rectum | A610298 | Biochain | Rectum | PM | M/27 |
| 9-Am-Stomach | 110P04A | Ambion | Stomach | PM | M/16 |
| 10-B-Stomach | A501159 | Biochain | Stomach | PM | M/24 |
| 11-B-Esophagus | A603814 | Biochain | Esophagus | PM | M/26 |
| 12-B-Esophagus | A603813 | Biochain | Esophagus | PM | M/41 |
| 13-Am-Pancreas | 071P25C | Ambion | Pancreas | PM | M/25 |
| 14-CG-Pancreas | CG-255-2 | Ichilov | Pancreas | PM | M/75 |
| 15-B-Lung | A409363 | Biochain | Lung | PM | F/26 |
| 16-Am-Lung (L93) | 111P0103A | Ambion | Lung | PM | F/61 |
| 17-B-Lung (L92) | A503204 | Biochain | Lung | PM | M/28 |
| 18-Am-Ovary (O47) | 061P43A | Ambion | Ovary | PM | F/16 |
| 19-B-Ovary (O48) | A504087 | Biochain | Ovary | PM | F/51 |
| 20-B-Ovary (O46) | A504086 | Biochain | Ovary | PM | F/41 |
| 21-Am-Cervix | 101P0101A | Ambion | Cervix | PM | F/40 |
| 22-B-Cervix | A408211 | Biochain | Cervix | PM | F/36 |
| 23-B-Cervix | A504089 | Biochain | Cervix | PM-Pool of 5 | M&F |
| 24-B-Uterus | A411074 | Biochain | Uterus | PM-Pool of 10 | M&F |
| 25-B-Uterus | A409248 | Biochain | Uterus | PM | F/43 |
| 26-B-Uterus | A504090 | Biochain | Uterus | PM-Pool of 5 | M&F |
| 27-B-Bladder | A501157 | Biochain | Bladder | PM | M/29 |
| 28-Am-Bladder | 071P02C | Ambion | Bladder | PM | M/20 |
| 29-B-Bladder | A504088 | Biochain | Bladder | PM-Pool of 5 | M&F |
| 30-Am-Placenta | 021P33A | Ambion | Placenta | PB | F/33 |
| 31-B-Placenta | A410165 | Biochain | Placenta | PB | F/26 |
| 32-B-Placenta | A411073 | Biochain | Placenta | PB-Pool of 5 | M&F |
| 33-B-Breast (B59) | A607155 | Biochain | Breast | PM | F/36 |
| 34-Am-Breast (B63) | 26486 | Ambion | Breast | PM | F/43 |
| 35-Am-Breast (B64) | 23036 | Ambion | Breast | PM | F/57 |
| 36-Cl-Prostate (P53) | 1070317 | Clontech | Prostate | PB-Pool of 47 | M&F |
| 37-Am-Prostate (P42) | 061P04A | Ambion | Prostate | PM | M/47 |
| 38-Am-Prostate (P59) | 25955 | Ambion | Prostate | PM | M/62 |
| 39-Am-Testis | 111P0104A | Ambion | Testis | PM | M/25 |
| 40-B-Testis | A411147 | Biochain | Testis | PM | M/74 |
| 41-Cl-Testis | 1110320 | Clontech | Testis | PB-Pool of 45 | M&F |
| 42-CG-Adrenal | CG-184-10 | Ichilov | Adrenal | PM | F/81 |
| 43-B-Adrenal | A610374 | Biochain | Adrenal | PM | F/83 |
| 44-B-Heart | A411077 | Biochain | Heart | PB-Pool of 5 | M&F |
| 45-CG-Heart | CG-255-9 | Ichilov | Heart | PM | M/75 |
| 46-CG-Heart | CG-227-1 | Ichilov | Heart | PM | F/36 |
| 47-Am-Liver | 081P0101A | Ambion | Liver | PM | M/64 |
| 48-CG-Liver | CG-93-3 | Ichilov | Liver | PM | F/19 |
| 49-CG-Liver | CG-124-4 | Ichilov | Liver | PM | F/34 |
| 50-Cl-BM | 1110932 | Clontech | Bone Marrow | PM-Pool of 8 | M&F |
| 51-CGEN-Blood | WBC#5 | CGEN | Blood | | M |
| 52-CGEN-Blood | WBC#4 | CGEN | Blood | | M |
| 53-CGEN-Blood | WBC#3 | CGEN | Blood | | M |
| 54-CG-Spleen | CG-267 | Ichilov | Spleen | PM | F/25 |
| 55-CG-Spleen | 111P0106B | Ambion | Spleen | PM | M/25 |
| 56-CG-Spleen | A409246 | Biochain | Spleen | PM | F/12 |
| 56-CG-Thymus | CG-98-7 | Ichilov | Thymus | PM | F/28 |
| 58-Am-Thymus | 101P0101A | Ambion | Thymus | PM | M/14 |
| 59-B-Thymus | A409278 | Biochain | Thymus | PM | M/28 |
| 60-B-Thyroid | A610287 | Biochain | Thyroid | PM | M/27 |
| 61-B-Thyroid | A610286 | Biochain | Thyroid | PM | M/24 |
| 62-CG-Thyroid | CG-119-2 | Ichilov | Thyroid | PM | F/66 |
| 63-Cl-Salivary Gland | 1070319 | Clontech | Salivary Gland | PM-Pool of 24 | M&F |
| 64-Am-Kidney | 111P0101B | Ambion | Kidney | PM-Pool of 14 | M&F |
| 65-Cl-Kidney | 1110970 | Clontech | Kidney | PM-Pool of 14 | M&F |
| 66-B-Kidney | A411080 | Biochain | Kidney | PM-Pool of 5 | M&F |
| 67-CG-Cerebellum | CG-183-5 | Ichilov | Cerebellum | PM | M/74 |
| 68-CG-Cerebellum | CG-212-5 | Ichilov | Cerebellum | PM | M/54 |
| 69-B-Brain | A411322 | Biochain | Brain | PM | M/28 |
| 70-Cl-Brain | 1120022 | Clontech | Brain | PM-Pool of 2 | M&F |
| 71-B-Brain | A411079 | Biochain | Brain | PM-Pool of 2 | M&F |
| 72-CG-Brain | CG-151-1 | Ichilov | Brain | PM | F/86 |
| 73-Am-Skeletal Muscle | 101P013A | Ambion | Skeletal Muscle | PM | F/28 |
| 74-Cl-Skeletal Muscle | 1061038 | Clontech | Skeletal Muscle | PM-Pool of 2 | M&F |

TABLE 1_5
Tissue samples in normal panel:

| | | Sample id(GCI)/ | Tissue id (GCI)/ | Sample id |
|---|---|---|---|---|
| | | case id | Specimen id | (Asterand)/RNA id |
| sample name | Source | (Asterand) Lot no. | (Asternd) | (GCI) |
| 1-(7)-Bc-Rectum | Biochain | A610297 | | |
| 2-(8)-Bc-Rectum | Biochain | A610298 | | |
| 3-GC-Colon | GCI | CDSUV | CDSUVNR3 | |
| 4-As-Colon | Asterand | 16364 | 31802 | 31802B1 |
| 5-As-Colon | Asterand | 22900 | 74446 | 74446B1 |
| 6-GC-Small bowl | GCI | V9L7D | V9L7DN6Z | |
| 7-GC-Small bowl | GCI | M3GVT | M3GVTN5R | |
| 8-GC-Small bowl | GCI | 196S2 | 196S2AJN | |
| 9-(9)-Am-Stomach | Ambion | 110P04A | | |
| 10-(10)-Bc-Stomach | Biochain | A501159 | | |
| 11-(11)-Bc-Esoph | Biochain | A603814 | | |
| 12-(12)-Bc-Esoph | Biochain | A603813 | | |
| 13-As-Panc | Asterand | 8918 | 9442 | 9442C1 |
| 14-As-Panc | Asterand | 10082 | 11134 | 11134B1 |
| 15-(48)-Ic-Liver | Ichilov | CG-93-3 | | |
| 16-As-Liver | Asterand | 7916 | 7203 | 7203B1 |
| 17-(28)-Am-Bladder | Ambion | 071P02C | | |
| 18-(29)-Bc-Bladder | Biochain | A504088 | | |
| 19-(64)-Am-Kidney | Ambion | 111P0101B | | |
| 20-(65)-Cl-Kidney | Clontech | 1110970 | | |
| 21-(66)-Bc-Kidney | Biochain | A411080 | | |
| 22-GC-Kidney | GCI | N1EVZ | N1EVZN91 | |
| 23-GC-Kidney | GCI | BMI6W | BMI6WN9F | |
| 24-(42)-Ic-Adrenal | Ichilov | CG-184-10 | | |
| 25-(43)-Bc-Adrenal | Biochain | A610374 | | |
| 26-(16)-Am-Lung | Ambion | 111P0103A | | |
| 27-(17)-Bc-Lung | Biochain | A503204 | | |
| 28-As-Lung | Asterand | 9078 | 9275 | 9275B1 |
| 29-As-Lung | Asterand | 6692 | 6161 | 6161A1 |
| 30-As-Lung | Asterand | 7900 | 7180 | 7180F1 |
| 31-(75)-GC-Ovary | GCI | L629FRV1 | | |
| 32-(76)-GC-Ovary | GCI | DWHTZRQX | | |
| 33-(77)-GC-Ovary | GCI | FDPL9NJ6 | | |
| 34-(78)-GC-Ovary | GCI | GWXUZN5M | | |
| 35-(21)-Am-Cerix | Ambion | 101P0101A | | |
| 36-GC-cervix | GCI | E2P2N | E2P2NAP4 | |
| 37-(24)-Bc-Uterus | Biochain | A411074 | | |
| 38-(26)-Bc-Uterus | Biochain | A504090 | | |
| 39-(30)-Am-Placen | Ambion | 021P33A | | |
| 40-(32)-Bc-Placen | Biochain | A411073 | | |
| 41-GC-Breast | GCI | DHLR1 | | |
| 42-GC-Breast | GCI | TG6J6 | | |
| 43-GC-Breast | GCI | E6UDD | E6UDDNCF | |
| 44-(38)-Am-Prostate | Ambion | 25955 | | |
| 45-Bc-Prostate | Biochain | A609258 | | |
| 46-As-Testis | Asterand | 13071 | 19567 | 19567B1 |
| 47-As-Testis | Asterand | 19671 | 42120 | 42120A1 |
| 48-GC-Artery | GCI | 7FUUP | 7FUUPAMP | |
| 49-GC-Artery | GCI | YGTVY | YGTVYAIN | |
| 50-Th-Blood-PBMC | Tel-Hashomer | 52497 | | |
| 51-Th-Blood-PBMC | Tel-Hashomer | 31055 | | |
| 52-Th-Blood-PBMC | Tel-Hashomer | 31058 | | |
| 53-(54)-Ic-Spleen | Ichilov | CG-267 | | |
| 54-(55)-Ic-Spleen | Ichilov | 111P0106B | | |
| 55-(57)-Ic-Thymus | Ichilov | CG-98-7 | | |
| 56-(58)-Am-Thymus | Ambion | 101P0101A | | |
| 57-(60)-Bc-Thyroid | Biochain | A610287 | | |
| 58-(62)-Ic-Thyroid | Ichilov | CG-119-2 | | |
| 59-Gc-Sali gland | GCI | NNSMV | NNSMVNJC | |
| 60-(67)-Ic-Cerebellum | Ichilov | CG-183-5 | | |
| 61-(68)-Ic-Cerebellum | Ichilov | CG-212-5 | | |
| 62-(69)-Bc-Brain | Biochain | A411322 | | |
| 63-(71)-Bc-Brain | Biochain | A411079 | | |
| 64-(72)-Ic-Brain | Ichilov | CG-151-1 | | |
| 65-(44)-Bc-Heart | Biochain | A411077 | | |
| 66-(46)-Ic-Heart | Ichilov | CG-227-1 | | |
| 67-(45)-Ic-Heart | Ichilov | CG-255-9 | | |
| (Fibrotic) | | | | |
| 68-GC-Skel Mus | GCI | T8YZS | T8YZSN7O | |
| 69-GC-Skel Mus | GCI | Q3WKA | Q3WKANCJ | |
| 70-As-Skel Mus | Asterand | 8774 | 8235 | 8235G1 |
| 71-As-Skel Mus | Asterand | 8775 | 8244 | 8244A1 |
| 72-As-Skel Mus | Asterand | 10937 | 12648 | 12648C1 |
| 73-As-Skel Mus | Asterand | 6692 | 6166 | 6166A1 |

### Materials and Experimental Procedures

RNA preparation—RNA was obtained from Clontech (Franklin Lakes, N.J. USA 07417, www.clontech.com), BioChain Inst. Inc. (Hayward, Calif. 94545 USA www.biochain.com), ABS (Wilmington, Del. 19801, USA, http://www.absbioreagents.com) or Ambion (Austin, Tex. 78744 USA, http://www.ambion.com). Alternatively, RNA was generated from tissue samples using TRI-Reagent (Molecular Research Center), according to Manufacturer's instructions. Tissue and RNA samples were obtained from patients or from postmortem. Total RNA samples were treated with DNaseI (Ambion) and purified using RNeasy columns (Qiagen).

RT PCR—Purified RNA (1 μg) was mixed with 150 ng Random Hexamer primers (Invitrogen) and 500 μM dNTP in a total volume of 15.6 μl. The mixture was incubated for 5 min at 65° C. and then quickly chilled on ice. Thereafter, 5 μl of 5× SuperscriptII first strand buffer (Invitrogen), 2.4 μl 0.1M DTT and 40 units RNasin (Promega) were added, and the mixture was incubated for 10 min at 25° C., followed by further incubation at 42° C. for 2 min. Then, 1 μl (200 units) of SuperscriptII (Invitrogen) was added and the reaction (final volume of 25 μl) was incubated for 50 min at 42° C. and then inactivated at 70° C. for 15 min. The resulting cDNA was diluted 1:20 in TE buffer (10 mM Tris pH=8, 1 mM EDTA pH=8).

Real-Time RT-PCR analysis—cDNA (5 μl), prepared as described above, was used as a template in Real-Time PCR reactions using the SYBR Green I assay (PE Applied Biosystem) with specific primers and UNG Enzyme (Eurogentech or ABI or Roche). The amplification was effected as follows: 50° C. for 2 min, 95° C. for 10 min, and then 40 cycles of 95° C. for 15 sec, followed by 60° C. for 1 min. Detection was performed by using the PE Applied Biosystem SDS 7000. The cycle in which the reactions achieved a threshold level (Ct) of fluorescence was registered and was used to calculate the relative transcript quantity in the RT reactions. The relative quantity was calculated using the equation Q=efficiencŷ^{-Ct}. The efficiency of the PCR reaction was calculated from a standard curve, created by using serial dilutions of several reverse transcription (RT) reactions. To minimize inherent differences in the RT reaction, the resulting relative quantities were normalized to normalization factor calculated in one of the following methods as indicated in the text:

Method 1—the geometric mean of the relative quantities of the selected housekeeping (HSKP) genes was used as normalization factor.

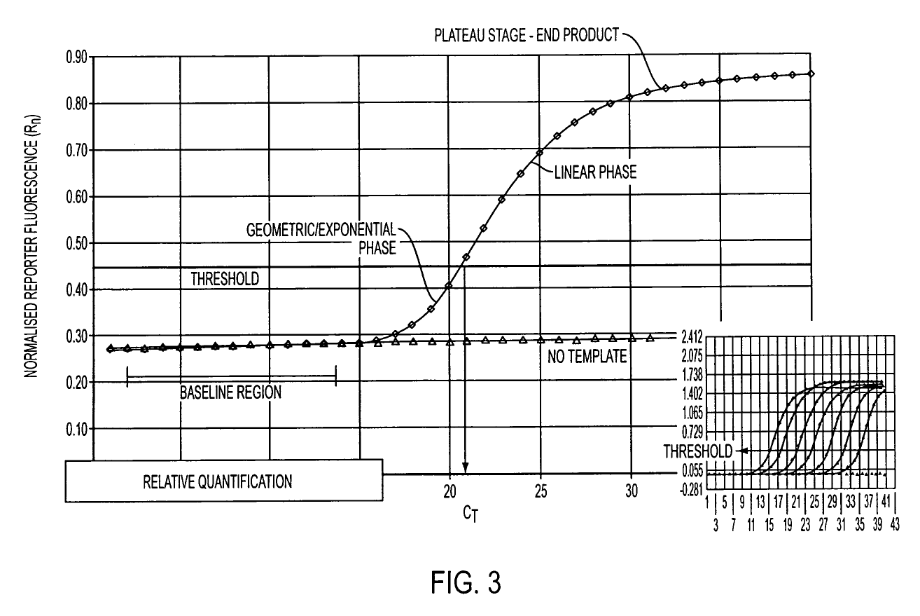

Method 2—The expression of several housekeeping (HSKP) genes was checked on every panel. The relative quantity (Q) of each housekeeping gene in each sample, calculated as described above, was divided by the median quantity of this gene in all panel samples to obtain the “relative Q rel to MED”. Then, for each sample the median of the “relative Q rel to MED” of the selected housekeeping genes was calculated and served as normalization factor of this sample for further calculations. Schematic summary of quantitative real-time PCR analysis is presented in FIG. 3. As shown, the x-axis shows the cycle number. The CT=Threshold Cycle point, which is the cycle that the amplification curve crosses the fluorescence threshold that was set in the experiment. This point is a calculated cycle number in which PCR products signal is above the background level (passive dye ROX) and still in the Geometric/Exponential phase (as shown, once the level of fluorescence crosses the measurement threshold, it has a geometrically increasing phase, during which measurements are most accurate, followed by a linear phase and a plateau phase; for quantitative measurements, the latter two phases do not provide accurate measurements). The y-axis shows the normalized reporter fluorescence. It should be noted that this type of analysis provides relative quantification.

Unless defined otherwise, the normalization of the Real-Time RT-PCR analysis results described herein was carried out according to method 1 above.

The sequences of the housekeeping genes measured in all the examples on tissue testing panel were as follows:

PBGD (GenBank Accession No. BC019323)
(SEQ ID NO:1576),
PBGD Forward primer (SEQ ID NO: 529):
TGAGAGTGATTCGCGTGGG
PBGD Reverse primer (SEQ ID NO: 530):
CCAGGGTACGAGGCTTTCAAT
PBGD-amplicon (SEQ ID NO: 531):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194)
(SEQ ID NO: 1577),
HPRT1 Forward primer (SEQ ID NO: 532):
TGACACTGGCAAAACAATGCA
HPRT1 Reverse primer (SEQ ID NO: 533):
GGTCCTTTTCACCAGCAAGCT
HPRT1-amplicon (SEQ ID NO: 612):
TGACACTGGCAAAACAATGCAGACTTTGCTTTCCTTGGTCAGGCAGTATA
ATCCAAAGATGGTCAAGGTCGCAAGCTTGCTGGTGAAAAGGACC
G6PD (GenBank Accession No. NM_000402)
(SEQ ID NO:1578)
G6PD Forward primer (SEQ ID NO: 613):
gaggccgtcaccaagaacat
G6PD Reverse primer (SEQ ID NO: 614):
ggacagccggtcagagctc
G6PD-amplicon (SEQ ID NO: 615):
gaggccgtcaccaagaacattcacgagtcctgcatgagccagataggctg
gaaccgcatcatcgtggagaagcccttcgggagggacctgcagagctctg
accggctgtcc
RPS27A (GenBank Accession No. NM_002954)
(SEQ ID NO:1579)
RPS27A Forward primer (SEQ ID NO: 642):
CTGGCAAGCAGCTGGAAGAT
RPS27A Reverse primer (SEQ ID NO:1260):
TTTCTTAGCACCACCACGAAGTC
RPS27A-amplicon (SEQ ID NO: 1261):
CTGGCAAGCAGCTGGAAGATGGACGTACTTTGTCTGACTACAATATTCAA
AAGGAGTCTACTCTTCATCTTGTGTTGAGACTTCGTGGTGGTGCTAAGAA
A

The sequences of the housekeeping genes measured in all the examples on normal tissue panel were as follows:

RPL19 (GenBank Accession No. NM_000981)
(SEQ ID NO: 1580),
RPL19 Forward primer (SEQ ID NO: 1262):
TGGCAAGAAGAAGGTCTGGTTAG
RPL19 Reverse primer (SEQ ID NO: 1263):
TGATCAGCCCATCTTTGATGAG
RPL19 amplicon (SEQ ID NO: 1264):
TGGCAAGAAGAAGGTCTGGTTAGACCCCAATGAGACCAATGAAATCGCCA
ATGCCAACTCCCGTCAGCAGATCCGGAAGCTCATCAAAGATGGGCTGATC
A
TATA box (GenBank Accession No. NM_003194)
(SEQ ID NO: 1581),
TATA box Forward primer (SEQ ID NO: 1265):
CGGTTTGCTGCGGTAATCAT
TATA box Reverse primer (SEQ ID NO: 1266):
TTTCTTGCTGCCAGTCTGGAC
TATA box amplicon (SEQ ID NO: 1267):
CGGTTTGCTGCGGTAATCATGAGGATAAGAGAGCCACGAACCACGGCACT
GATTTTCAGTTCTGGGAAAATGGTGTGCACAGGAGCCAAGAGTGAAGAAC
AGTCCAGACTGGCAGCAAGAAA
Ubiquitin (GenBank Accession No. BC000449)
(SEQ ID NO: 1582)
Uhiquitin Forward primer (SEQ ID NO: 1268):
ATTTGGGTCGCGGTTCTTG
Ubiquitin Reverse primer (SEQ ID NO: 1269):
TGCCTTGACATTCTCGATGGT
Ubiquitin amplicon (SEQ ID NO: 1270):
ATTTGGGTCGCGGTTCTTGTTTGTGGATCGCTGTGATCGTCACTTGACAA
TGCAGATCTTCGTGAAGACTCTGACTGGTAAGACCATCACCCTCGAGG T
TGAGCCCAGTGACACCATCGAGAATGTCAAGGCA
SDHA (GenBank Accession No. NM_004168)
(SEQ ID NO: 1583)
SDHA Forward primer (SEQ ID NO: 1271):
TGGGAACAAGAGGGCATCTG
SDHA Reverse primer (SEQ ID NO: 1272):
CCACCACTGCATCAAATTCATG
SDHA-amplicon (SEQ ID NO: 1273):
TGGGAACAAGAGGGCATCTGCTAAAGTTTCAGATTCCATTTCTGCTCAGT
ATCCAGTAGTGGATCATGAATTTGATGCAGTGGTGG

Oligonucleotide-Based Micro-Array Experiment Protocol—

### Microarray Fabrication

Microarrays (chips) were printed by pin deposition using the MicroGrid II MGII 600 robot from BioRobtics Limited (Cambridge, UK). 50-mer oligonucleotides target sequences were designed by Compugen Ltd (Tel-Aviv, Ill.) as described by A. Shoshan et al, “Optical technologies and informatics”, Proceedings of SPIE. Vol 4266, pp. 86-95 (2001). The designed oligonucleotides were synthesized and purified by desalting with the Sigma-Genosys system (The Woodlands, Tex., US) and all of the oligonucleotides were joined to a C6 amino-modified linker at the 5′ end, or being attached directly to CodeLink slides (Cat #25-6700-01. Amersham Bioscience, Piscataway, N.J., US). The 50-mer oligonucleotides, forming the target sequences, were first suspended in Ultra-pure DDW (Cat # 01-866-1A Kibbutz Beit-Haemek, Israel) to a concentration of 50 μM. Before printing the slides, the oligonucleotides were resuspended in 300 mM sodium phosphate (pH 8.5) to final concentration of 150 mM and printed at 35-40% relative humidity at 21° C.

Each slide contained a total of 9792 features in 32 subarrays. Of these features, 4224 features were sequences of interest according to the present invention and negative controls that were printed in duplicate. An additional 288 features (96 target sequences printed in triplicate) contained housekeeping genes from Human Evaluation Library2, Compugen Ltd, Israel. Another 384 features are E. coli spikes 1-6, which are oligos to E-Coli genes which are commercially available in the Array Control product (Array control-sense oligo spots, Ambion Inc. Austin, Tex. Cat #1781, Lot #112K06).

### Post-Coupling Processing of Printed Slides

After the spotting of the oligonucleotides to the glass (CodeLink) slides, the slides were incubated for 24 hours in a sealed saturated NaCl humidification chamber (relative humidity 70-75%).

Slides were treated for blocking of the residual reactive groups by incubating them in blocking solution at 50° C. for 15 minutes (10 ml/slide of buffer containing 0.1M Tris, 50 mM ethanolamine, 0.1% SDS). The slides were then rinsed twice with Ultra-pure DDW (double distilled water). The slides were then washed with wash solution (10 ml/slide. 4×SSC, 0.1% SDS)) at 50° C. for 30 minutes on the shaker. The slides were then rinsed twice with Ultra-pure DDW, followed by drying by centrifugation for 3 minutes at 800 rpm.

Next, in order to assist in automatic operation of the hybridization protocol, the slides were treated with Ventana Discovery hybridization station barcode adhesives. The printed slides were loaded on a Bio-Optica (Milan, Italy) hematology staining device and were incubated for 10 minutes in 50 ml of 3-Aminopropyl Triethoxysilane (Sigma A3648 lot #122K589). Excess fluid was dried and slides were then incubated for three hours in 20 mm/Hg in a dark vacuum desiccator (Pelco 2251, Ted Pella, Inc. Redding Calif.).

The following protocol was then followed with the Genisphere 900-RP (random primer), with mini elute columns on the Ventana Discovery HybStation™, to perform the microarray experiments. Briefly, the protocol was performed as described with regard to the instructions and information provided with the device itself. The protocol included cDNA synthesis and labeling. cDNA concentration was measured with the TBS-380 (Turner Biosystems. Sunnyvale, Calif.) PicoFlour, which is used with the OliGreen ssDNA Quantitation reagent and kit.

Hybridization was performed with the Ventana Hybridization device, according to the provided protocols (Discovery Hybridization Station Tuscon Ariz.).

The slides were then scanned with GenePix 4000B dual laser scanner from Axon Instruments Inc, and analyzed by GenePix Pro 5.0 software.

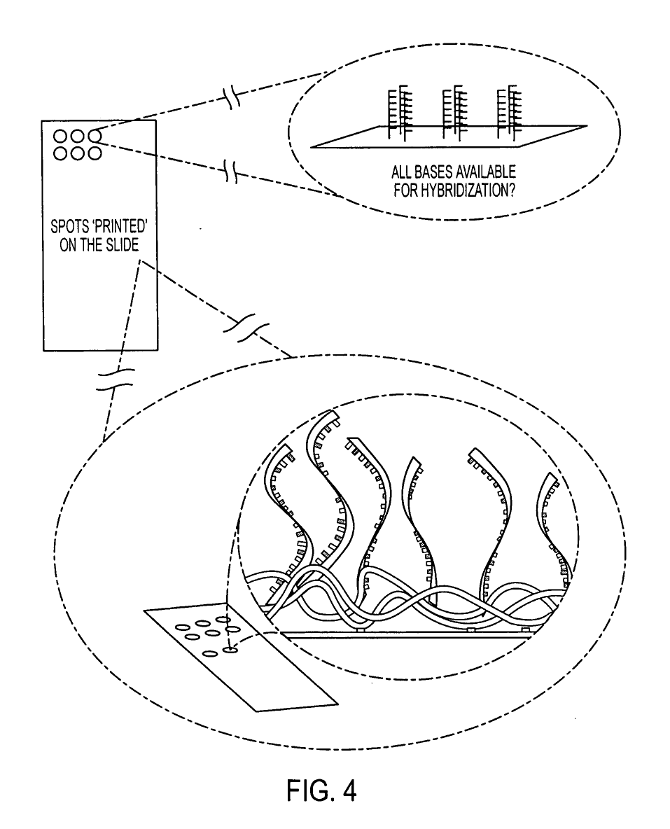

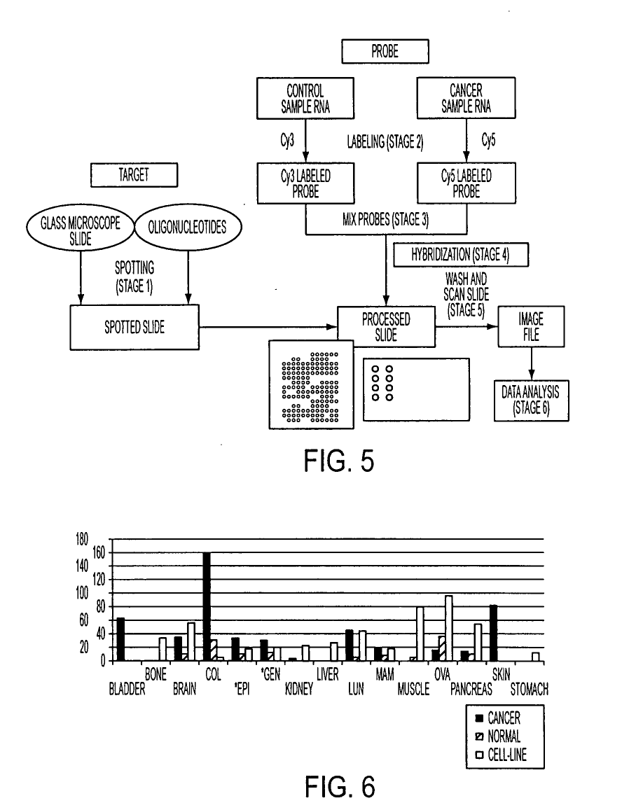

Schematic summary of the oligonucleotide based microarray fabrication and the experimental flow is presented in FIGS. 4 and 5.

Briefly, as shown in FIG. 4, DNA oligonucleotides at 25 uM were deposited (printed) onto Amersham ‘CodeLink’ glass slides generating a well defined ‘spot’. These slides are covered with a long-chain, hydrophilic polymer chemistry that creates an active 3-D surface that covalently binds the DNA oligonucleotides 5′-end via the C6-amine modification. This binding ensures that the full length of the DNA oligonucleotides is available for hybridization to the cDNA and also allows lower background, high sensitivity and reproducibility.

FIG. 5 shows a schematic method for performing the microarray experiments. It should be noted that stages on the left-hand or right-hand side may optionally be performed in any order, including in parallel, until stage 4 (hybridization). Briefly, on the left-hand side, the target oligonucleotides are being spotted on a glass microscope slide (although optionally other materials could be used) to form a spotted slide (stage 1). On the right hand side, control sample RNA and cancer sample RNA are Cy3 and Cy5 labeled, respectively (stage 2), to form labeled probes. It should be noted that the control and cancer samples come from corresponding tissues (for example, normal prostate tissue and cancerous prostate tissue). Furthermore, the tissue from which the RNA was taken is indicated below in the specific examples of data for particular clusters, with regard to overexpression of an oligonucleotide from a “chip” (microarray), as for example “prostate” for chips in which prostate cancerous tissue and normal tissue were tested as described above. In stage 3, the probes are mixed. In stage 4, hybridization is performed to form a processed slide. In stage 5, the slide is washed and scanned to form an image file, followed by data analysis in stage 6.

### Description for Cluster M85491

Cluster M85491 features 2 transcript(s) and 11 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| M85491_PEA_1_T16 | 1 |
| M85491_PEA_1_T20 | 2 |

TABLE 2
Segments of interest

| Segment Name | SEQ ID NO: |
|---|---|
| M85491_PEA_1_node_0 | 89 |
| M85491_PEA_1_node_13 | 90 |
| M85491_PEA_1_node_21 | 91 |
| M85491_PEA_1_node_23 | 92 |
| M85491_PEA_1_node_24 | 93 |
| M85491_PEA_1_node_8 | 94 |
| M85491_PEA_1_node_9 | 95 |
| M85491_PEA_1_node_10 | 96 |
| M85491_PEA_1_node_18 | 97 |
| M85491_PEA_1_node_19 | 98 |
| M85491_PEA_1_node_6 | 99 |

TABLE 3
Proteins of interest

| Protein Name | SEQ ID NO: |
|---|---|
| M85491_PEA_1_P13 | 534 |
| M85491_PEA_1_P14 | 535 |

These sequences are variants of the known protein Ephrin type-B receptor 2 [precursor] (SwissProt accession identifier EPB2_HUMAN; known also according to the synonyms EC 2.7.1.112; Tyrosine-protein kinase receptor EPH-3; DRT; Receptor protein-tyrosine kinase HEK5; ERK), SEQ ID NO: 616, referred to herein as the previously known protein.

Protein Ephrin type-B receptor 2 [precursor] is known or believed to have the following function(s): Receptor for members of the ephrin-B family. The sequence for protein Ephrin type-B receptor 2 [precursor] is given at the end of the application, as “Ephrin type-B receptor 2 [precursor] amino acid sequence” (SEQ ID NO:616). Known polymorphisms for this sequence are as shown in Table 4.

TABLE 4
Amino acid mutations for Known Protein

| SNP position(s) on | |
|---|---|
| amino acid sequence | Comment |
| 671 | A -> R./FTId = VAR_004162. |
| 1-20 | MALRRLGAALLLLPLLAAVE -> |
| | MWVPVLALPVCTYA |
| 923 | E -> 22K |
| 956 | L -> 22V |
| 958 | V -> 22L |
| 154 | G -> 22D |
| 476 | K -> 22KQ |
| 495-496 | Missing |
| 532 | E -> 22D |
| 568 | R -> 22RR |
| 589 | M -> 22I |
| 788 | I -> 22F |
| 853 | S -> 22A |

Protein Ephrin type-B receptor 2 [precursor] (SEQ ID NO:616) localization is believed to be Type I membrane protein.

The following GO Annotation(s) apply to the previously known protein. The following annotation(s) were found: protein amino acid phosphorylation; transmembrane receptor protein tyrosine kinase signaling pathway; neurogenesis, which are annotation(s) related to Biological Process; protein tyrosine kinase; receptor; transmembrane-ephrin receptor; ATP binding; transferase, which are annotation(s) related to Molecular Function; and integral membrane protein, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster M85491 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 6 and Table 5. This cluster is overexpressed (at least at a minimum level) in the following pathological conditions: epithelial malignant tumors and a mixture of malignant tumors from different tissues.

TABLE 5
Normal tissue distribution

| Name of Tissue | Number |
|---|---|
| bladder | 0 |
| Bone | 0 |
| Brain | 10 |
| Colon | 31 |
| epithelial | 10 |
| general | 12 |
| Kidney | 0 |
| Liver | 0 |
| Lung | 5 |
| Breast | 8 |
| Muscle | 5 |
| Ovary | 36 |
| pancreas | 10 |
| Skin | 0 |
| stomach | 0 |

TABLE 6
P values and ratios for expression in cancerous tissue

| Name of Tissue | P1 | P2 | SP1 | R3 | SP2 | R4 |
|---|---|---|---|---|---|---|
| bladder | 5.4e−01 | 6.0e−01 | 3.2e−01 | 2.5 | 4.6e−01 | 1.9 |
| Bone | 1 | 2.8e−01 | 1 | 1.0 | 7.0e−01 | 1.8 |
| Brain | 3.4e−01 | 3.6e−01 | 1.2e−01 | 2.9 | 1.8e−02 | 2.7 |
| Colon | 3.4e−02 | 5.7e−02 | 8.2e−02 | 2.8 | 2.0e−01 | 2.1 |
| epithelial | 1.7e−03 | 3.5e−03 | 2.0e−03 | 2.8 | 1.1e−02 | 2.2 |
| general | 4.8e−04 | 5.2e−04 | 6.7e−04 | 2.3 | 1.3e−03 | 1.9 |
| Kidney | 4.3e−01 | 3.7e−01 | 1 | 1.1 | 7.0e−01 | 1.5 |
| Liver | 1 | 4.5e−01 | 1 | 1.0 | 6.9e−01 | 1.5 |
| Lung | 2.2e−01 | 2.7e−01 | 6.9e−02 | 3.6 | 3.4e−02 | 3.6 |
| Breast | 8.2e−01 | 7.3e−01 | 6.9e−01 | 1.2 | 6.8e−01 | 1.2 |
| Muscle | 9.2e−01 | 4.8e−01 | 1 | 0.8 | 1.5e−01 | 3.2 |
| Ovary | 8.5e−01 | 7.3e−01 | 9.0e−01 | 0.7 | 6.7e−01 | 1.0 |
| pancreas | 5.5e−01 | 2.0e−01 | 6.7e−01 | 1.2 | 3.5e−01 | 1.8 |
| Skin | 2.9e−01 | 4.7e−01 | 1.4e−01 | 7.0 | 6.4e−01 | 1.6 |
| stomach | 1.5e−01 | 3.2e−01 | 1 | 1.0 | 8.0e−01 | 1.3 |

As noted above, cluster M85491 features 2 transcript(s), which were listed in Table 1 above. These transcript(s) encode for protein(s) which are variant(s) of protein Ephrin type-B receptor 2 [precursor]. A description of each variant protein according to the present invention is now provided.

Variant protein M85491_PEA_{—}1_P13 (SEQ ID NO:534) according to the present invention has an amino acid sequence as given at the end of the application; it is encoded by transcript(s) M85491_PEA_{—}1_T16 (SEQ ID NO:1). An alignment is given to the known protein (Ephrin type-B receptor 2 [precursor]) at the end of the application. One or more alignments to one or more previously published protein sequences are given at the end of the application. A brief description of the relationship of the variant protein according to the present invention to each such aligned protein is as follows:

Comparison Report Between M85491_PEA_{—}1_P13 (SEQ ID NO:534) and EPB2_HUMAN (SEQ ID NO:616):

1. An isolated chimeric polypeptide encoding for M85491_PEA_{—}1_P13 (SEQ ID NO:534), comprising a first amino acid sequence being at least 90% homologous to MALRRLGAALLLLPLLAAVEETLMDSTTATAELGWMVHPPSGWEEVSGYDENMNTIRTYQVCNVFESSQ NNWLRTKFIRRRGAHRIHVEMKFSVRDCSSIPSVPGSCKETFNLYYYEADFDSATKTFPNWMENPWVKVD TIAADESFSQVDLGGRVMKINTEVRSFGPVSRSGFYLAFQDYGGCMSLIAVRVFYRKCPRIIQNGAIFQETL SGAESTSLVAARGSCIANAEEVDVPIKLYCNGDGEWLVPIGRCMCKAGFEAVENGTVCRGCPSGTFKANQ GDEACTHCPINSRTTSEGATNCVCRNGYYRADLDPLDMPCTTIPSAPQAVISSVNETSLMLEWTPPRDSGG REDLVYNIICKSCGSGRGACTRCGDNVQYAPRQLGLTEPRIYISDLLAHTQYTFEIQAVNGVTDQSPFSPQF ASVNITTNQAAPSAVSIMHQVSRTVDSITLSWSQPDQPNGVILDYELQYYEK corresponding to amino acids 1-476 of EPB2_HUMAN (SEQ ID NO:616), which also corresponds to amino acids 1-476 of M85491_PEA_{—}1_P13 (SEQ ID NO:534), and a second amino acid sequence being at least 70%, optionally at least 80%, preferably at least 85%, more preferably at least 90% and most preferably at least 95% homologous to a polypeptide having the sequence VPIGWVLSPSPTSLRAPLPG (SEQ ID NO:1480) corresponding to amino acids 477-496 of M85491_PEA_{—}1_P13 (SEQ ID NO:534), wherein said first and second amino acid sequences are contiguous and in a sequential order.

2. An isolated polypeptide encoding for a tail of M85491_PEA_{—}1_P13 (SEQ ID NO:534), comprising a polypeptide being at least 70%, optionally at least about 80%, preferably at least about 85%, more preferably at least about 90% and most preferably at least about 95% homologous to the sequence VPIGWVLSPSPTSLRAPLPG (SEQ ID NO:1480) in M85491_PEA_{—}1_P13 (SEQ ID NO:534).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein M85491_PEA_{—}1_P13 (SEQ ID NO:534) is encoded by the following transcript(s): M85491_PEA_{—}1_T16 (SEQ ID NO:1), for which the sequence(s) is/are given at the end of the application. The coding portion of transcript M85491_PEA_{—}1_T16 (SEQ ID NO:1) is shown in bold; this coding portion starts at position 143 and ends at position 1630. The transcript also has the following SNPs as listed in Table 7 (given according to their position on the nucleotide sequence, with the alternative nucleic acid listed; the last column indicates whether the SNP is known or not; the presence of known SNPs in variant protein M85491_PEA_{—}1_P13 (SEQ ID NO:534) sequence provides support for the deduced sequence of this variant protein according to the present invention).

TABLE 7
Nucleic acid SNPs

| SNP position on nucleotide | Alternative | Previously |
|---|---|---|
| sequence | nucleic acid | known SNP? |
| 799 | G -> A | Yes |
| 1066 | C -> T | Yes |
| 1519 | A -> G | Yes |
| 1872 | C -> T | Yes |
| 2044 | T -> C | Yes |
| 2156 | G -> A | Yes |
| 2606 | C -> A | Yes |
| 2637 | G -> C | Yes |

Variant protein M85491_PEA_{—}1_P14 (SEQ ID NO:535) according to the present invention has an amino acid sequence as given at the end of the application; it is encoded by transcript(s) M85491_PEA_{—}1_T20 (SEQ ID NO:2). An alignment is given to the known protein (Ephrin type-B receptor 2 [precursor]) at the end of the application. One or more alignments to one or more previously published protein sequences are given at the end of the application. A brief description of the relationship of the variant protein according to the present invention to each such aligned protein is as follows:

Comparison Report Between M85491_PEA_{—}1_P14 (SEQ ID NO:535) and EPB2_HUMAN (SEQ ID NO:616):

1. An isolated chimeric polypeptide encoding for M85491_PEA_{—}1_P14 (SEQ ID NO:535), comprising a first amino acid sequence being at least 90% homologous to MALRRLGAALLLLPLLAAVEETLMDSTTATAELGWMVHPPSGWEEVSGYDENMNTIRTYQVCNVFESSQ NNWLRTKFIRRRGAHRIHVEMKFSVRDCSSIPSVPGSCKETFNLYYYEADFDSATKTFPNWMENPWVKVD TIAADESFSQVDLGGRVMKINTEVRSFGPVSRSGFYLAFQDYGGCMSLIAVRVFYRKCPRIIQNGAIFQETL SGAESTSLVAARGSCIANAEEVDVPIKLYCNGDGEWLVPIGRCMCKAGFEAVENGTVCR corresponding to amino acids 1-270 of EPB2_HUMAN (SEQ ID NO:616), which also corresponds to amino acids 1-270 of M85491_PEA_{—}1_P14 (SEQ ID NO:535), and a second amino acid sequence being at least 70%, optionally at least 80%, preferably at least 85%, more preferably at least 90% and most preferably at least 95% homologous to a polypeptide having the sequence ERQDLTMLSRLVLNSWPQMILPPQPPKVLEL (SEQ ID NO:1481) corresponding to amino acids 271-301 of M85491_PEA_{—}1_P14 (SEQ ID NO:535), wherein said first and second amino acid sequences are contiguous and in a sequential order.

2. An isolated polypeptide encoding for a tail of M85491_PEA_{—}1_P14 (SEQ ID NO:535), comprising a polypeptide being at least 70%, optionally at least about 80%, preferably at least about 85%, more preferably at least about 90% and most preferably at least about 95% homologous to the sequence ERQDLTMLSRLVLNSWPQMILPPQPPKVLEL (SEQ ID NO:1481) in M85491_PEA_{—}1_P14 (SEQ ID NO:535). The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein M85491_PEA_{—}1_P14 (SEQ ID NO:535) is encoded by the following transcript(s): M85491_PEA_{—}1_T20 (SEQ ID NO:2), for which the sequence(s) is/are given at the end of the application. The coding portion of transcript M85491_PEA_{—}1_T20 (SEQ ID NO:2) is shown in bold; this coding portion starts at position 143 and ends at position 1045. The transcript also has the following SNPs as listed in Table 8 (given according to their position on the nucleotide sequence, with the alternative nucleic acid listed; the last column indicates whether the SNP is known or not; the presence of known SNPs in variant protein M85491_PEA_{—}1_P14 (SEQ ID NO:535) sequence provides support for the deduced sequence of this variant protein according to the present invention).

TABLE 8
Nucleic acid SNPs

| SNP position on nucleotide | Alternative | Previously |
|---|---|---|
| sequence | nucleic acid | known SNP? |
| 799 | G -> A | Yes |
| 1135 | T -> C | Yes |
| 1160 | T -> C | Yes |
| 1172 | A -> C | Yes |
| 1176 | T -> A | Yes |

As noted above, cluster M85491 features 11 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster M85491_PEA_{—}1_node_{—}0 (SEQ ID NO:89) according to the present invention is supported by 5 libraries. The number of libraries was determined as previously described. This segment can be found in the following transcript(s): M85491_PEA_{—}1_T16 (SEQ ID NO:1) and M85491_PEA_{—}1_T20 (SEQ ID NO:2). Table 9 below describes the starting and ending position of this segment on each transcript.

TABLE 9
Segment location on transcripts

| | Segment starting | Segment ending |
|---|---|---|
| Transcript name | position | position |
| M85491_PEA_1_T16 (SEQ ID | 1 | 203 |
| NO: 1) | | |
| M85491_PEA_1_T20 (SEQ ID | 1 | 203 |
| NO: 2) | | |

Segment cluster M85491_PEA_{—}1_node_{—}13 (SEQ ID NO:90) according to the present invention is supported by 6 libraries. The number of libraries was determined as previously described. This segment can be found in the following transcript(s): M85491_PEA_{—}1_T20 (SEQ ID NO:2). Table 10 below describes the starting and ending position of this segment on each transcript.

TABLE 10
Segment location on transcripts

| | Segment starting | Segment ending |
|---|---|---|
| Transcript name | position | position |
| M85491_PEA_1_T20 (SEQ ID | 954 | 1182 |
| NO: 2) | | |

Microarray (chip) data is also available for this segment as follows. As described above with regard to the cluster itself, various oligonucleotides were tested for being differentially expressed in various disease conditions, particularly cancer. The following oligonucleotides were found to hit this segment, shown in Table 11.

TABLE 11
Oligonucleotides related to this segment

| | Overexpressed | Chip |
|---|---|---|
| Oligonucleotide name | in cancers | reference |
| M85491_0_0_25999 | colorectal cancer | Colon |
| (SEQ ID NO: 1398) | | |

Segment cluster M85491_PEA_{—}1_node_{—}21 (SEQ. ID NO:91) according to the present invention is supported by 18 libraries. The number of libraries was determined as previously described. This segment can be found in the following transcript(s): M85491_PEA_{—}1_T16 (SEQ ID NO:1). Table 12 below describes the starting and ending position of this segment on each transcript.

TABLE 12
Segment location on transcripts

| | Segment | |
|---|---|---|
| Transcript name | starting position | Segment ending position |
| M85491_PEA_1_T16 | 1110 | 1445 |
| (SEQ ID NO: 1) | | |

Segment cluster M85491_PEA_{—}1_node_{—}23 (SEQ ID NO:92) according to the present invention is supported by 18 libraries. The number of libraries was determined as previously described. This segment can be found in the following transcript(s): M85491_PEA_{—}1_T16 (SEQ ID NO:1). Table 13 below describes the starting and ending position of this segment on each transcript.

TABLE 13
Segment location on transcripts

| | Segment | |
|---|---|---|
| Transcript name | starting position | Segment ending position |
| M85491_PEA_1_T16 | 1446 | 1570 |
| (SEQ ID NO: 1) | | |

Segment cluster M85491_PEA_{—}1_node_{—}24 (SEQ ID NO:93) according to the present invention is supported by 3 libraries. The number of libraries was determined as previously described. This segment can be found in the following transcript(s): M85491_PEA_{—}1_T16 (SEQ ID NO:1). Table 14 below describes the starting and ending position of this segment on each transcript.

TABLE 14
Segment location on transcripts

| | Segment | |
|---|---|---|
| Transcript name | starting position | Segment ending position |
| M85491_PEA_1_T16 | 1571 | 2875 |
| (SEQ ID NO: 1) | | |

Segment cluster M85491_PEA_{—}1_node_{—}8 (SEQ ID NO:94) according to the present invention is supported by 25 libraries. The number of libraries was determined as previously described. This segment can be found in the following transcript(s): M85491_PEA_{—}1_T16 (SEQ ID NO:1) and M85491_PEA_{—}1_T20 (SEQ ID NO:2). Table 15 below describes the starting and ending position of this segment on each transcript.

TABLE 15
Segment location on transcripts

| | Segment | |
|---|---|---|
| | starting | Segment ending |
| Transcript name | position | position |
| M85491_PEA_1_T16 (SEQ ID NO: 1) | 269 | 672 |
| M85491_PEA_1_T20 (SEQ ID NO: 2) | 269 | 672 |

Microarray (chip) data is also available for this segment as follows. As described above with regard to the cluster itself, various oligonucleotides were tested for being differentially expressed in various disease conditions, particularly cancer. The following oligonucleotides were found to hit this segment with regard to colon cancer, shown in Table 16.

TABLE 16
Oligonucleotides related to this segment

| Oligonucleotide name | Overexpressed in cancers | Chip reference |
|---|---|---|
| M85491_0_14_0 | colorectal cancer | Colon |
| (SEQ ID NO: 1399) | | |

Segment cluster M85491_PEA_{—}1_node_{—}9 (SEQ ID NO:95) according to the present invention is supported by 20 libraries. The number of libraries was determined as previously described. This segment can be found in the following transcript(s): M85491_PEA_{—}1_T16 (SEQ ID NO:1) and M85491_PEA_{—}1_T20 (SEQ ID NO:2). Table 17 below describes the starting and ending position of this segment on each transcript.

TABLE 17
Segment location on transcripts

| | Segment | |
|---|---|---|
| | starting | Segment ending |
| Transcript name | position | position |
| M85491_PEA_1_T16 (SEQ ID NO: 1) | 673 | 856 |
| M85491_PEA_1_T20 (SEQ ID NO: 2) | 673 | 856 |

According to an optional embodiment of the present invention, short segments related to the above cluster are also provided. These segments are up to about 120 bp in length, and so are included in a separate description.

Segment cluster M85491_PEA_{—}1_node_{—}10 (SEQ ID NO:96) according to the present invention is supported by 17 libraries. The number of libraries was determined as previously described. This segment can be found in the following transcript(s): M85491_PEA_{—}1_T16 (SEQ ID NO:1) and M85491_PEA_{—}1_T20 (SEQ ID NO:2). Table 18 below describes the starting and ending position of this segment on each transcript.

TABLE 18
Segment location on transcripts

| | Segment | |
|---|---|---|
| | starting | Segment ending |
| Transcript name | position | position |
| M85491_PEA_1_T16 (SEQ ID NO: 1) | 857 | 953 |
| M85491_PEA_1_T20 (SEQ ID NO: 2) | 857 | 953 |

Segment cluster M85491_PEA_{—}1_node_{—}18 (SEQ ID NO:97) according to the present invention is supported by 15 libraries. The number of libraries was determined as previously described. This segment can be found in the following transcript(s): M85491_PEA_{—}1_T16 (SEQ ID NO:1). Table 19 below describes the starting and ending position of this segment on each transcript.

TABLE 19
Segment location on transcripts

| | Segment | |
|---|---|---|
| Transcript name | starting position | Segment ending position |
| M85491_PEA_1_T16 | 954 | 1044 |
| (SEQ ID NO: 1) | | |

Segment cluster M85491_PEA_{—}1_node_{—}19 (SEQ ID NO:98) according to the present invention is supported by 15 libraries. The number of libraries was determined as previously described. This segment can be found in the following transcript(s): M85491_PEA_{—}1_T16 (SEQ ID NO:1). Table 20 below describes the starting and ending position of this segment on each transcript.

TABLE 20
Segment location on transcripts

| | Segment | |
|---|---|---|
| Transcript name | starting position | Segment ending position |
| M85491_PEA_1_T16 | 1045 | 1109 |
| (SEQ ID NO: 1) | | |

Segment cluster M85491_PEA_{—}1_node_{—}6 (SEQ ID NO:99) according to the present invention is supported by 11 libraries. The number of libraries was determined as previously described. This segment can be found in the following transcript(s): M85491_PEA_{—}1_T16 (SEQ ID NO:1) and M85491_PEA_{—}1_T20 (SEQ ID NO:2). Table 21 below describes the starting and ending position of this segment on each transcript.

TABLE 21
Segment location on transcripts

| | Segment | |
|---|---|---|
| | starting | Segment ending |
| Transcript name | position | position |
| M85491_PEA_1_T16 (SEQ ID NO: 1) | 204 | 268 |
| M85491_PEA_1_T20 (SEQ ID NO: 2) | 204 | 268 |

### Variant Protein Alignment to the Previously Known Protein:

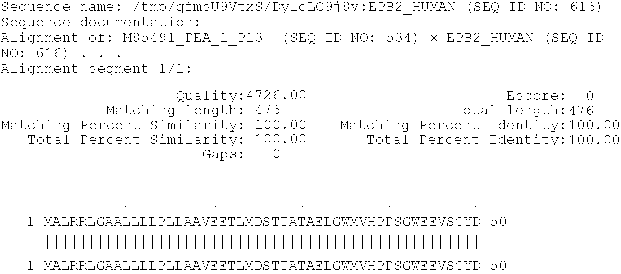

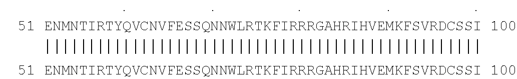

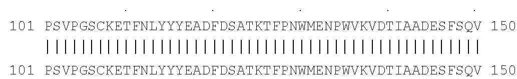

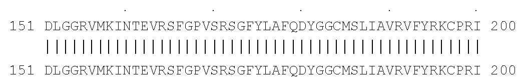

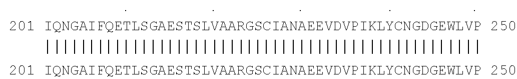

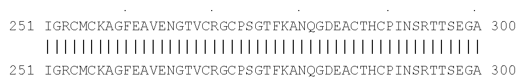

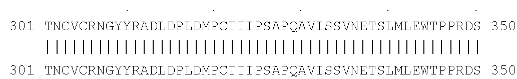

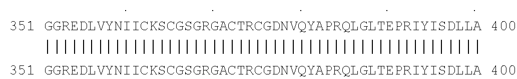

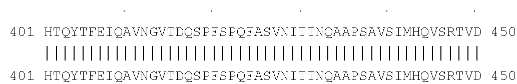

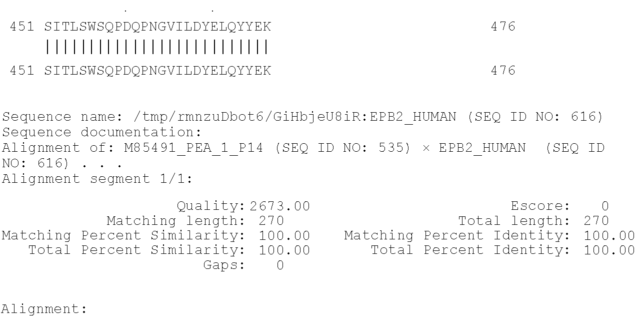

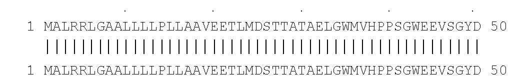

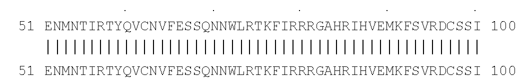

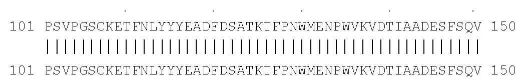

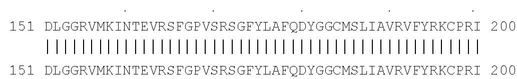

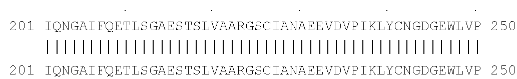

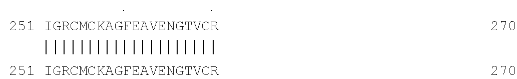

Expression of Ephrin Type-B Receptor 2 Precursor (EC 2.7.1.112) (Tyrosine-Protein Kinase Receptor EPH-3) M85491 Transcripts which are Detectable by Amplicon as Depicted in Sequence Name M85491Seg24 (SEQ ID NO:1276) in Normal and Cancerous Colon Tissues

Expression of Ephrin type-B receptor 2 precursor (EC 2.7.1.112) (Tyrosine-protein kinase receptor EPH-3) transcripts detectable by or according to seg24, M85491seg24 amplicon (SEQ ID NO:1276) and M85491seg24F (SEQ ID NO:1274) and M85491seg24R (SEQ ID NO:1275) primers was measured by real time PCR. In parallel the expression of four housekeeping genes —PBGD (GenBank Accession No. BC019323 (SEQ ID NO:1576); amplicon —PBGD-amplicon, SEQ ID NO:531), HPRT1 (GenBank Accession No. NM_{—}000194 (SEQ ID NO:1577); amplicon —HPRT1-amplicon, SEQ ID NO:612), G6PD (GenBank Accession No. NM_{—}000402 (SEQ ID NO:1578); G6PD amplicon, SEQ ID NO:615), and RPS27A (GenBank Accession No. NM_{—}002954 (SEQ ID NO:1579); RPS27A amplicon, SEQ ID NO:1261) was measured similarly. For each RT (RT-PCR) sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, “Tissue samples in testing panel”, above), to obtain a value of fold up-regulation for each sample relative to median of the normal PM samples.

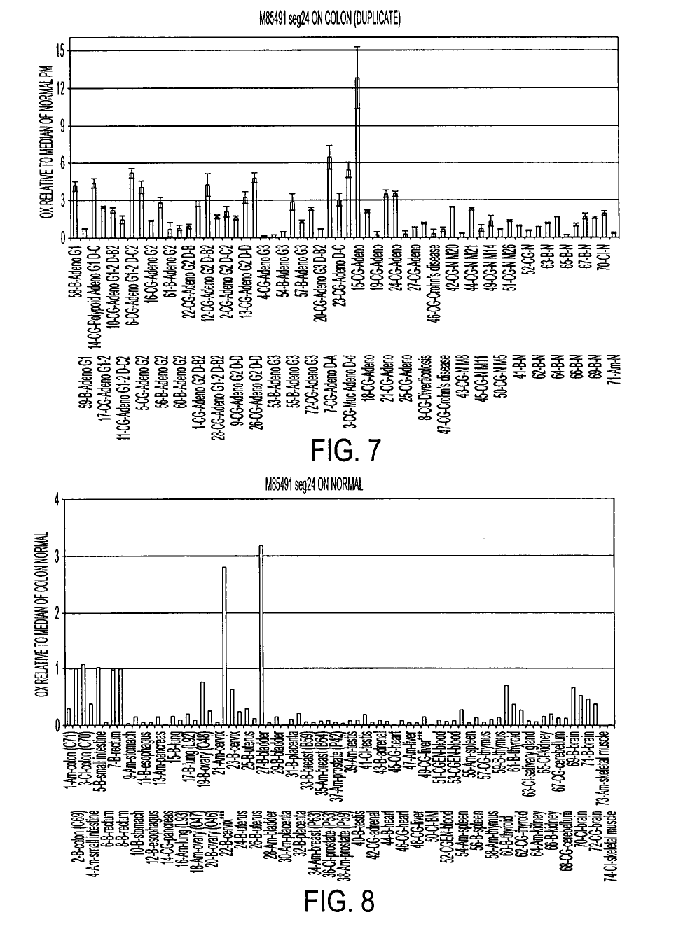

FIG. 7 is a histogram showing over expression of the above-indicated Ephrin type-B receptor 2 precursor (EC 2.7.1.112) (Tyrosine-protein kinase receptor EPH-3) transcripts in cancerous colon samples relative to the normal samples. Values represent the average of duplicate experiments. Error bars indicate the minimal and maximal values obtained.

As is evident from FIG. 7, the expression of Ephrin type-B receptor 2 precursor (EC 2.7.1.112) (Tyrosine-protein kinase receptor EPH-3) transcripts detectable by the above amplicon in cancer samples was significantly higher than in the non-cancerous samples (Sample Nos. 41, 52, 62-67, 69-71 Table 1, “Tissue samples in testing panel”). Notably over-expression of at least 3 fold was found in 13 out of 37 adenocarcinoma samples.

Statistical analysis was applied to verify the significance of these results, as described below.

The P value for the difference in the expression levels of Ephrin type-B receptor 2 precursor (EC 2.7.1.112) (Tyrosine-protein kinase receptor EPH-3) transcripts detectable by the above amplicon(s) in colon cancer samples versus the normal tissue samples was determined by T test as 6.83E-04 Threshold of 3 fold over expression was found to differentiate between cancer and normal samples with P value of 2.66E-02 in as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: M85491seg24F forward primer (SEQ ID NO:1274); and M85491seg24R reverse primer (SEQ ID NO:1275).

The present invention also preferably encompasses any amplicon obtained through the use of any suitable primer pair; for example, for the above experiment, the following amplicon was obtained as a non-limiting illustrative example only of a suitable amplicon: M85491seg24 (SEQ ID NO:1276).

M85491seg24F-

(SEQ ID NO: 1274)

GGCGTCTTTCTCCCTCTGAAC
M85491seg24R-

(SEQ ID NO: 1275)

GTCCCATTCTGGGTGCTGTG
M85491seg24-

(SEQ ID NO: 1276)

GGCGTCTTTCTCCCTCTGAACCTCAGTTTCCACCTGTGTCGAGTGTGGGT
GAGACCCCTCGCGGGGAGCTATGCAGGTTACGGAGAAAAGGCAGCACAGC
ACCCAGAATGGGAC

Expression of Ephrin Type-B Receptor 2 Precursor (EC 2.7.1.112) (Tyrosine-Protein Kinase Receptor EPH-3) M85491 Transcripts which are Detectable by Amplicon as Depicted in Sequence Name M85491Seg24 (SEQ. ID NO:1276) in Different Normal Tissues.
Expression of Ephrin type-B receptor 2 precursor transcripts detectable by or according to M85491 seg24 amplicon(s) (SEQ ID NO:1276) and M85491 seg24F (SEQ ID NO:1274) and M85491 seg24R (SEQ ID NO:1275) was measured by real time PCR. In parallel the expression of four housekeeping genes—RPL19 (GenBank Accession No. NM_{—}000981 (SEQ ID NO:1580); RPL19 amplicon), TATA box (GenBank Accession No. NM_{—}003194 (SEQ ID NO:1581); TATA amplicon), Ubiquitin (GenBank Accession No. BC000449 (SEQ ID NO:1582); amplicon-Ubiquitin-amplicon) and SDHA (GenBank Accession No. NM_{—}004168 (SEQ ID NO:1583); amplicon—SDHA-amplicon) was measured similarly. For each RT sample, the expression of the above amplicon was normalized to the geometric mean of the quantities of the housekeeping genes. The normalized quantity of each RT sample was then divided by the median of the quantities of the lung samples (Sample Nos. 15-17 Table 2 Tissue samples in normal panel), to obtain a value of relative expression of each sample relative to median of the lung samples.

The results are described in FIG. 8, presenting the histogram showing the expression of M85491 transcripts which are detectable by amplicon as depicted in sequence name M85491seg24 (SEQ ID NO:1276) in different normal tissues.

Forward primer (SEQ ID NO: 1274):
GGCGTCTTTCTCCCTCTGAAC
Reverse primer (SEQ ID NO: 1275):
GTCCCATTCTGGGTGCTGTG
Amplicon (SEQ ID NO: 1276):
GGCGTCTTTCTCCCTCTGAACCTCAGTTTCCACCTGTGTCGAGTGTGGGT
GAGACCCCTCGCGGGGAGCTATGCAGGTTACGGAGAAAAGGCAGCACAGC
ACCCAGAATGGGAC

### Description for Cluster T10888

Cluster T10888 features 4 transcript(s) and 8 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| T10888_PEA_1_T1 | 3 |
| T10888_PEA_1_T4 | 4 |
| T10888_PEA_1_T5 | 5 |
| T10888_PEA_1_T6 | 6 |

TABLE 2
Segments of interest

| Segment Name | SEQ ID NO: |
|---|---|
| T10888_PEA_1_node_11 | 100 |
| T10888_PEA_1_node_12 | 101 |
| T10888_PEA_1_node_17 | 102 |
| T10888_PEA_1_node_4 | 103 |
| T10888_PEA_1_node_6 | 104 |
| T10888_PEA_1_node_7 | 105 |
| T10888_PEA_1_node_9 | 106 |
| T10888_PEA_1_node_15 | 107 |

TABLE 3
Proteins of interest

| Protein Name | SEQ ID NO: |
|---|---|
| T10888_PEA_1_P2 | 536 |
| T10888_PEA_1_P4 | 537 |
| T10888_PEA_1_P5 | 538 |
| T10888_PEA_1_P6 | 539 |

These sequences are variants of the known protein Carcinoembryonic antigen-related cell adhesion molecule 6 precursor (SwissProt accession identifier CEA6_HUMAN; known also according to the synonyms Normal cross-reacting antigen; Nonspecific crossreacting antigen; CD66c antigen), SEQ ID NO: 617, referred to herein as the previously known protein.

The sequence for protein Carcinoembryonic antigen-related cell adhesion molecule 6 precursor (SEQ ID NO:617) is given at the end of the application, as “Carcinoembryonic antigen-related cell adhesion molecule 6 precursor amino acid sequence”. Known polymorphisms for this sequence are as shown in Table 4.

TABLE 4
Amino acid mutations for Known Protein

| SNP position(s) on amino | |
|---|---|
| acid sequence | Comment |
| 138 | F -> L |
| 239 | V -> G |

Protein Carcinoembryonic antigen-related cell adhesion molecule 6 precursor (SEQ ID NO:617) localization is believed to be Attached to the membrane by a GPI-anchor.

The previously known protein also has the following indication(s) and/or potential therapeutic use(s): Cancer. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Immunostimulant. A therapeutic role for a protein represented by the cluster has been predicted. The cluster was assigned this field because there was information in the drug database or the public databases (e.g., described herein above) that this protein, or part thereof, is used or can be used for a potential therapeutic indication: Imaging agent; Anticancer; Immunostimulant; Immunoconjugate; Monoclonal antibody, murine; Antisense therapy; antibody.

The following GO Annotation(s) apply to the previously known protein. The following annotation(s) were found: signal transduction; cell-cell signaling, which are annotation(s) related to Biological Process; and integral plasma membrane protein, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster T10888 can be used as a diagnostic marker according to overexpression of transcripts of this cluster in cancer. Expression of such transcripts in normal tissues is also given according to the previously described methods. The term “number” in the right hand column of the table and the numbers on the y-axis of the figure below refer to weighted expression of ESTs in each category, as “parts per million” (ratio of the expression of ESTs for a particular cluster to the expression of all ESTs in that category, according to parts per million).

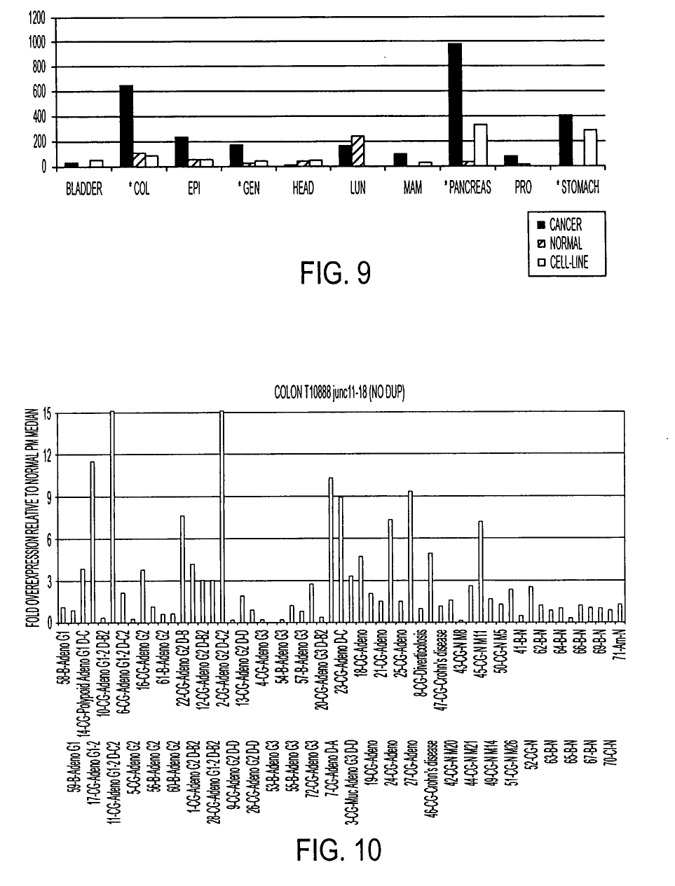

Overall, the following results were obtained as shown with regard to the histograms in FIG. 9 and Table 5. This cluster is overexpressed (at least at a minimum level) in the following pathological conditions: colorectal cancer, a mixture of malignant tumors from different tissues, pancreas carcinoma and gastric carcinoma.

TABLE 5
Normal tissue distribution

| Name of Tissue | Number |
|---|---|
| bladder | 0 |
| Colon | 107 |
| epithelial | 52 |
| general | 22 |
| head and neck | 40 |
| lung | 237 |
| breast | 0 |
| pancreas | 32 |
| prostate | 12 |
| stomach | 0 |

TABLE 6
P values and ratios for expression in cancerous tissue

| Name of Tissue | P1 | P2 | SP1 | R3 | SP2 | R4 |
|---|---|---|---|---|---|---|
| bladder | 5.4e−01 | 3.4e−01 | 5.6e−01 | 1.8 | 4.6e−01 | 1.9 |
| colon | 1.2e−01 | 1.7e−01 | 2.8e−05 | 3.7 | 7.9e−04 | 2.8 |
| epithelial | 3.3e−02 | 2.1e−01 | 2.8e−20 | 2.8 | 4.8e−10 | 1.9 |
| general | 3.3e−05 | 2.2e−03 | 1.9e−44 | 4.9 | 4.6e−27 | 3.3 |
| head and neck | 4.6e−01 | 4.3e−01 | 1 | 0.8 | 7.5e−01 | 1.0 |
| lung | 7.6e−01 | 8.2e−01 | 8.9e−01 | 0.6 | 1 | 0.3 |
| breast | 3.7e−02 | 4.1e−02 | 1.5e−01 | 3.3 | 3.1e−01 | 2.4 |
| pancreas | 2.6e−01 | 2.4e−01 | 8.6e−23 | 2.8 | 1.5e−19 | 4.5 |
| prostate | 9.1e−01 | 9.3e−01 | 4.1e−02 | 1.2 | 1.0e−01 | 1.0 |
| stomach | 4.5e−02 | 5.6e−02 | 5.1e−04 | 4.1 | 4.7e−04 | 6.3 |

As noted above, cluster T10888 features 4 transcript(s), which were listed in Table 1 above. These transcript(s) encode for protein(s) which are variant(s) of protein Carcinoembryonic antigen-related cell adhesion molecule 6 precursor (SEQ ID NO:617). A description of each variant protein according to the present invention is now provided.

Variant protein T10888_PEA_{—}1_P2 (SEQ ID NO:536) according to the present invention has an amino acid sequence as given at the end of the application; it is encoded by transcript(s) T10888_PEA_{—}1_T1 (SEQ ID NO:3). An alignment is given to the known protein (Carcinoembryonic antigen-related cell adhesion molecule 6 precursor (SEQ ID NO:617)) at the end of the application. One or more alignments to one or more previously published protein sequences are given at the end of the application. A brief description of the relationship of the variant protein according to the present invention to each such aligned protein is as follows:

Comparison Report Between T10888_PEA_{—}1_P2 (SEQ ID NO:536) and CEA6_HUMAN (SEQ ID NO:617):

1. An isolated chimeric polypeptide encoding for T10888_PEA_{—}1_P2 (SEQ ID NO:536), comprising a first amino acid sequence being at least 90% homologous to MGPPSAPPCRLHVPWKEVLLTASLLTFWNPPTTAKLTIESTPFNVAEGKEVLLLAHNLPQNRIGYSWYKGE RVDGNSLIVGYVIGTQQATPGPAYSGRETIYPNASLLIQNVTQNDTGFYTLQVIKSDLVNEEATGQFHVYP ELPKPSISSNNSNPVEDKDAVAFTCEPEVQNTTYLWWVNGQSLPVSPRLQLSNGNMTLTLLSVKRNDAGS YECEIQNPASANRSDPVTLNVLYGPDVPTISPSKANYRPGENLNLSCHAASNPPAQYSWFINGTFQQSTQEL FIPNITVNNSGSYMCQAHNSATGLNRTTVTMITVS corresponding to amino acids 1-319 of CEA6_HUMAN (SEQ ID NO:617), which also corresponds to amino acids 1-319 of T10888_PEA_{—}1_P2 (SEQ ID NO:536), and a second amino acid sequence being at least 70%, optionally at least 80%, preferably at least 85%, more preferably at least 90% and most preferably at least 95% homologous to a polypeptide having the sequence DWTRP (SEQ ID NO:1482) corresponding to amino acids 320-324 of T10888_PEA_{—}1_P2 (SEQ ID NO:536), wherein said first and second amino acid sequences are contiguous and in a sequential order.

2. An isolated polypeptide encoding for a tail of T10888_PEA_{—}1_P2 (SEQ ID NO:536), comprising a polypeptide being at least 70%, optionally at least about 80%, preferably at least about 85%, more preferably at least about 90% and most preferably at least about 95% homologous to the sequence DWTRP (SEQ ID NO:1482) in T10888_PEA_{—}1_P2 (SEQ ID NO:536).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein T10888_PEA_{—}1_P2 (SEQ ID NO:536) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein T10888_PEA_{—}1_P2 (SEQ ID NO:536) sequence provides support for the deduced sequence of this variant protein according to the present invention).

TABLE 7
Amino acid mutations

| SNP position(s) on amino | Alternative | Previously |
|---|---|---|
| acid sequence | amino acid(s) | known SNP? |
| 13 | V -> | No |
| 232 | N -> D | No |
| 324 | P -> | No |
| 63 | I -> | No |
| 92 | G -> | No |

Variant protein T10888_PEA_{—}1_P2 (SEQ ID NO:536) is encoded by the following transcript(s): T10888_PEA_{—}1_T1 (SEQ ID NO:3), for which the sequence(s) is/are given at the end of the application. The coding portion of transcript T10888_PEA_{—}1_T1 (SEQ ID NO:3) is shown in bold; this coding portion starts at position 151 and ends at position 1122. The transcript also has the following SNPs as listed in Table 8 (given according to their position on the nucleotide sequence, with the alternative nucleic acid listed; the last column indicates whether the SNP is known or not; the presence of known SNPs in variant protein T10888_PEA_{—}1_P2 (SEQ ID NO:536) sequence provides support for the deduced sequence of this variant protein according to the present invention).

TABLE 8
Nucleic acid SNPs

| SNP position on | Alternative | Previously |
|---|---|---|
| nucleotide sequence | nucleic acid | known SNP? |
| 119 | C -> T | No |
| 120 | A -> T | No |
| 1062 | A -> G | Yes |
| 1120 | C -> | No |
| 1297 | G -> T | Yes |
| 1501 | A -> G | Yes |
| 1824 | G -> A | No |
| 2036 | A -> C | No |
| 2036 | A -> G | No |
| 2095 | A -> C | No |
| 2242 | A -> C | No |
| 2245 | A -> C | No |
| 189 | C -> | No |
| 2250 | A -> T | Yes |
| 2339 | C -> A | Yes |
| 276 | G -> A | Yes |
| 338 | T -> | No |
| 424 | G -> | No |
| 546 | A -> G | No |
| 702 | C -> T | No |
| 844 | A -> G | No |
| 930 | C -> T | Yes |

Variant protein T10888_PEA_{—}1_P4 (SEQ ID NO:537) according to the present invention has an amino acid sequence as given at the end of the application; it is encoded by transcript(s) T10888_PEA_{—}1_T4 (SEQ ID NO:4). An alignment is given to the known protein (Carcinoembryonic antigen-related cell adhesion molecule 6 precursor (SEQ ID NO:617)) at the end of the application. One or more alignments to one or more previously published protein sequences are given at the end of the application. A brief description of the relationship of the variant protein according to the present invention to each such aligned protein is as follows:

Comparison Report Between T10888_PEA_{—}1_P4 (SEQ ID NO:537) and CEA6_HUMAN (SEQ ID NO:617):

1. An isolated chimeric polypeptide encoding for T10888_PEA_{—}1_P4 (SEQ ID NO:537), comprising a first amino acid sequence being at least 90% homologous to MGPPSAPPCRLHVPWKEVLLTASLLTFWNPPTTAKLTIESTPFNVAEGKEVLLLAHNLPQNRIGYSWYKGE RVDGNSLIVGYVIGTQQATPGPAYSGRETIYPNASLLIQNVTQNDTGFYTLQVIKSDLVNEEATGQFHVYP ELPKPSISSNNSNPVEDKDAVAFTCEPEVQNTTYLWWVNGQSLPVSPRLQLSNGNMTLTLLSVKRNDAGS YECEIQNPASANRSDPVTLNVL corresponding to amino acids 1-234 of CEA6_HUMAN (SEQ ID NO:617), which also corresponds to amino acids 1-234 of T10888_PEA_{—}1_P4 (SEQ ID NO:537), and a second amino acid sequence being at least 70%, optionally at least 80%, preferably at least 85%, more preferably at least 90% and most preferably at least 95% homologous to a polypeptide having the sequence LLLSSQLWPPSASRLECWPGWL (SEQ ID NO:1483) corresponding to amino acids 235-256 of T10888_PEA_{—}1_P4 (SEQ ID NO:537), wherein said first and second amino acid sequences are contiguous and in a sequential order.

2. An isolated polypeptide encoding for a tail of T10888_PEA_{—}1_P4 (SEQ ID NO:537) comprising a polypeptide being at least 70%, optionally at least about 80%, preferably at least about 85%, more preferably at least about 90% and most preferably at least about 95% homologous to the sequence LLLSSQLWPPSASRLECWPGWL (SEQ ID NO:1483) in T10888_PEA_{—}1_P4 (SEQ ID NO:537).

Comparison Report Between T10888_PEA_{—}1_P4 (SEQ ID NO:537) and Q13774 (SEQ ID NO:1382):

1. An isolated chimeric polypeptide encoding for T10888_PEA_{—}1_P4 (SEQ ID NO:537), comprising a first amino acid sequence being at least 90% homologous to MGPPSAPPCRLHVPWKEVLLTASLLTFWNPPTTAKLTIESTPFNVAEGKEVLLLAHNLPQNRIGYSWYKGE RVDGNSLIVGYVIGTQQATPGPAYSGRETIYPNASLLIQNVTQNDTGFYTLQVIKSDLVNEEATGQFHVYP ELPKPSISSNNSNPVEDKDAVAFTCEPEVQNTTYLWWVNGQSLPVSPRLQLSNGNMTLTLLSVKRNDAGS YECEIQNPASANRSDPVTLNVL corresponding to amino acids 1-234 of Q13774 (SEQ ID NO:1382), which also corresponds to amino acids 1-234 of T10888_PEA_{—}1_P4 (SEQ ID NO:537), and a second amino acid sequence being at least 70%, optionally at least 80%, preferably at least 85%, more preferably at least 90% and most preferably at least 95% homologous to a polypeptide having the sequence LLLSSQLWPPSASRLECWPGWL (SEQ ID NO:1483) corresponding to amino acids 235-256 of T10888_PEA_{—}1_P4 (SEQ ID NO:537), wherein said first and second amino acid sequences are contiguous and in a sequential order.

2. An isolated polypeptide encoding for a tail of T10888_PEA_{—}1_P4 (SEQ ID NO:537), comprising a polypeptide being at least 70%, optionally at least about 80%, preferably at least about 85%, more preferably at least about 90% and most preferably at least about 95% homologous to the sequence LLLSSQLWPPSASRLECWPGWL (SEQ ID NO:1483) in T10888_PEA_{—}1_P4 (SEQ ID NO:537).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein T10888_PEA_{—}1_P4 (SEQ ID NO:537) also has the following non-silent SNPs (Single Nucleotide Polymorphisms) as listed in Table 9, (given according to their position(s) on the amino acid sequence, with the alternative amino acid(s) listed; the last column indicates whether the SNP is known or not; the presence of known SNPs in variant protein T10888_PEA_{—}1_P4 (SEQ ID NO:537) sequence provides support for the deduced sequence of this variant protein according to the present invention).

TABLE 9
Amino acid mutations

| SNP position(s) on amino | Alternative | Previously |
|---|---|---|
| acid sequence | amino acid(s) | known SNP? |
| 13 | V -> | No |
| 232 | N -> D | No |
| 63 | I -> | No |
| 92 | G -> | No |

Variant protein T10888_PEA_{—}1_P4 (SEQ ID NO:537) is encoded by the following transcript(s): T10888_PEA_{—}1_T4 (SEQ ID NO:4), for which the sequence(s) is/are given at the end of the application. The coding portion of transcript T10888_PEA_{—}1_T4 (SEQ ID NO:4) is shown in bold; this coding portion starts at position 151 and ends at position 918. The transcript also has the following SNPs as listed in Table 10 (given according to their position on the nucleotide sequence, with the alternative nucleic acid listed; the last column indicates whether the SNP is known or not; the presence of known SNPs in variant protein T10888_PEA_{—}1_P4 (SEQ ID NO:537) sequence provides support for the deduced sequence of this variant protein according to the present invention).

TABLE 10
Nucleic acid SNPs

| SNP position on | Alternative | Previously |
|---|---|---|
| nucleotide sequence | nucleic acid | known SNP? |
| 119 | C -> T | No |
| 120 | A -> T | No |
| 978 | C -> | No |
| 1155 | G -> T | Yes |
| 1359 | A -> G | Yes |
| 1682 | G -> A | No |
| 1894 | A -> C | No |
| 1894 | A -> G | No |
| 1953 | A -> C | No |
| 2100 | A -> C | No |
| 2103 | A -> C | No |
| 2108 | A -> T | Yes |
| 189 | C -> | No |
| 2197 | C -> A | Yes |
| 276 | G -> A | Yes |
| 338 | T -> | No |
| 424 | G -> | No |
| 546 | A -> G | No |
| 702 | C -> T | No |
| 844 | A -> G | No |
| 958 | G -> | No |

Variant protein T10888_PEA_{—}1_P5 (SEQ ID NO:538) according to the present invention has an amino acid sequence as given at the end of the application; it is encoded by transcript(s) T10888_PEA_{—}1_T5 (SEQ ID NO:5). An alignment is given to the known protein (Carcinoembryonic antigen-related cell adhesion molecule 6 precursor (SEQ ID NO:617)) at the end of the application. One or more alignments to one or more previously published protein sequences are given at the end of the application. A brief description of the relationship of the variant protein according to the present invention to each such aligned protein is as follows:

Comparison Report Between T10888_PEA_{—}1_P5 (SEQ ID NO:538) and CEA6_HUMAN (SEQ ID NO:617):

1. An isolated chimeric polypeptide encoding for T10888_PEA_{—}1_P5 (SEQ ID NO:538), comprising a first amino acid sequence being at least 90% homologous to MGPPSAPPCRLHVPWKEVLLTASLLTFWNPPTTAKLTIESTPFNVAEGKEVLLLAHNLPQNRIGYSWYKGE RVDGNSLIVGYVIGTQQATPGPAYSGRETIYPNASLLIQNVTQNDTGFYTLQVIKSDLVNEEATGQFHVYP ELPKPSISSNNSNPVEDKDAVAFTCEPEVQNTTYLWWVNGQSLPVSPRLQLSNGNMTLTLLSVKRNDAGS YECEIQNPASANRSDPVTLNVLYGPDVPTISPSKANYRPGENLNLSCHAASNPPAQYSWFINGTFQQSTQEL FIPNITVNNSGSYMCQAHNSATGLNRTTVTMITVSG corresponding to amino acids 1-320 of CEA6_HUMAN (SEQ ID NO:617), which also corresponds to amino acids 1-320 of T10888_PEA_{—}1_P5 (SEQ ID NO:538), and a second amino acid sequence being at least 70%, optionally at least 80%, preferably at least 85%, more preferably at least 90% and most preferably at least 95% homologous to a polypeptide having the sequence KWIHEALASHFQVESGSQRRARKKFSFPTCVQGAHANPKFSPEPSQFTSADSFPLVFLFFVVFCFLISHV (SEQ ID NO:1484) corresponding to amino acids 321-390 of T10888_PEA_{—}1_P5 (SEQ ID NO:538), wherein said first and second amino acid sequences are contiguous and in a sequential order.

2. An isolated polypeptide encoding for a tail of T10888_PEA_{—}1_P5 (SEQ ID NO:538), comprising a polypeptide being at least 70%, optionally at least about 80%, preferably at least about 85%, more preferably at least about 90% and most preferably at least about 95% homologous to the sequence KWIHEALASHFQVESGSQRRARKKFSFPTCVQGAHANPKFSPEPSQFTSADSFPLVFLFFVVFCFLISHV (SEQ ID NO:1484) in T10888_PEA_{—}1_P5 (SEQ ID NO:538).

The location of the variant protein was determined according to results from a number of different software programs and analyses, including analyses from SignalP and other specialized programs. The variant protein is believed to be located as follows with regard to the cell: membrane. The protein localization is believed to be membrane because although both signal-peptide prediction programs agree that this protein has a signal peptide, both trans-membrane region prediction programs predict that this protein has a trans-membrane region downstream of this signal peptide.

Variant protein T10888_PEA_{—}1_P5 (SEQ ID NO:538) also has the following non-silent SNPs (Single Nucleotide Polymorphisms) as listed in Table 11, (given according to their position(s) on the amino acid sequence, with the alternative amino acid(s) listed; the last column indicates whether the SNP is known or not; the presence of known SNPs in variant protein T10888_PEA_{—}1_P5 (SEQ ID NO:538) sequence provides support for the deduced sequence of this variant protein according to the present invention).

TABLE 11
Amino acid mutations

| SNP position(s) on amino | Alternative | Previously |
|---|---|---|
| acid sequence | amino acid(s) | known SNP? |
| 13 | V -> | No |
| 232 | N -> D | No |
| 63 | I -> | No |
| 92 | G -> | No |

Variant protein T10888_PEA_{—}1_P5 (SEQ ID NO:538) is encoded by the following transcript(s): T10888_PEA_{—}1_T5 (SEQ ID NO:5), for which the sequence(s) is/are given at the end of the application. The coding portion of transcript T10888_PEA_{—}1_T5 (SEQ ID NO:5) is shown in bold; this coding portion starts at position 151 and ends at position 1320. The transcript also has the following SNPs as listed in Table 12 (given according to their position on the nucleotide sequence, with the alternative nucleic acid listed; the last column indicates whether the SNP is known or not; the presence of known SNPs in variant protein T10888_PEA_{—}1_P5 (SEQ ID NO:538) sequence provides support for the deduced sequence of this variant protein according to the present invention).

TABLE 12
Nucleic acid SNPs

| SNP position on | Alternative | Previously |
|---|---|---|
| nucleotide sequence | nucleic acid | known SNP? |
| 119 | C -> T | No |
| 120 | A -> T | No |
| 1062 | A -> G | Yes |
| 1943 | C -> A | Yes |
| 2609 | C -> T | Yes |
| 2647 | C -> G | No |
| 2701 | C -> T | Yes |
| 2841 | T -> C | Yes |
| 189 | C -> | No |
| 276 | G -> A | Yes |
| 338 | T -> | No |
| 424 | G -> | No |
| 546 | A -> G | No |
| 702 | C -> T | No |
| 844 | A -> G | No |
| 930 | C -> T | Yes |

Variant protein T10888_PEA_{—}1_P6 (SEQ ID NO:539) according to the present invention has an amino acid sequence as given at the end of the application; it is encoded by transcript(s) T10888_PEA_{—}1_T6 (SEQ ID NO:6). An alignment is given to the known protein (Carcinoembryonic antigen-related cell adhesion molecule 6 precursor (SEQ ID NO:617)) at the end of the application. One or more alignments to one or more previously published protein sequences are given at the end of the application.

Comparison Report Between T10888_PEA_{—}1_P6 (SEQ ID NO:539) and CEA6_HUMAN (SEQ ID NO:617):

1. An isolated chimeric polypeptide encoding for T10888_PEA_{—}1_P6 (SEQ ID NO:539), comprising a first amino acid sequence being at least 90% homologous to MGPPSAPPCRLHVPWKEVLLTASLLTFWNPPTTAKLTIESTPFNVAEGKEVLLLAHNLPQNRIGYSWYKGE RVDGNSLIVGYVIGTQQATPGPAYSGRETIYPNASLLIQNVTQNDTGFYTLQVIKSDLVNEEATGQFHVY corresponding to amino acids 1-141 of CEA6_HUMAN (SEQ ID NO:617), which also corresponds to amino acids 1-141 of T10888_PEA_{—}1_P6 (SEQ ID NO:539), and a second amino acid sequence being at least 70%, optionally at least 80%, preferably at least 85%, more preferably at least 90% and most preferably at least 95% homologous to a polypeptide having the sequence REYFHMTSGCWGSVLLPTYGIVRPGLCLWPSLHYILYQGLDI (SEQ ID NO:1485) corresponding to amino acids 142-183 of T10888_PEA_{—}1_P6 (SEQ ID NO:539), wherein said first and second amino acid sequences are contiguous and in a sequential order.

2. An isolated polypeptide encoding for a tail of T10888_PEA_{—}1_P6 (SEQ ID NO:539), comprising a polypeptide being at least 70%, optionally at least about 80%, preferably at least about 85%, more preferably at least about 90% and most preferably at least about 95% homologous to the sequence REYFHMTSGCWGSVLLPTYGIVRPGLCLWPSLHYILYQGLDI (SEQ ID NO:1485) in T10888_PEA_{—}1_P6 (SEQ ID NO:539).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein T10888_PEA_{—}1_P6 (SEQ ID NO:539) also has the following non-silent SNPs (Single Nucleotide Polymorphisms) as listed in Table 13, (given according to their position(s) on the amino acid sequence, with the alternative amino acid(s) listed; the last column indicates whether the SNP is known or not; the presence of known SNPs in variant protein T10888_PEA_{—}1_P6 (SEQ ID NO:539) sequence provides support for the deduced sequence of this variant protein according to the present invention).

TABLE 13
Amino acid mutations

| SNP position(s) on amino | Alternative | Previously |
|---|---|---|
| acid sequence | amino acid(s) | known SNP? |
| 13 | V -> | No |
| 63 | I -> | No |
| 92 | G -> | No |

Variant protein T10888_PEA_{—}1_P6 (SEQ ID NO:539) is encoded by the following transcript(s): T10888_PEA_{—}1_T6 (SEQ ID NO:6), for which the sequence(s) is/are given at the end of the application. The coding portion of transcript T10888_PEA_{—}1_T6 (SEQ ID NO:6) is shown in bold; this coding portion starts at position 151 and ends at position 699. The transcript also has the following SNPs as listed in Table 14 (given according to their position on the nucleotide sequence, with the alternative nucleic acid listed; the last column indicates whether the SNP is known or not; the presence of known SNPs in variant protein T10888_PEA_{—}1_P6 (SEQ ID NO:539) sequence provides support for the deduced sequence of this variant protein according to the present invention).

TABLE 14
Nucleic acid SNPs

| SNP position on | Alternative | Previously |
|---|---|---|
| nucleotide sequence | nucleic acid | known SNP? |
| 119 | C -> T | No |
| 120 | A -> T | No |
| 189 | C -> | No |
| 276 | G -> A | Yes |
| 338 | T -> | No |
| 424 | G -> | No |
| 546 | A -> G | No |

As noted above, cluster T10888 features 8 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster T10888_PEA_{—}1_node_{—}11 (SEQ ID NO:100) according to the present invention is supported by 57 libraries. The number of libraries was determined as previously described. This segment can be found in the following transcript(s): T10888_PEA_{—}1_T1 (SEQ ID NO:3) and T10888_PEA_{—}1_T5 (SEQ ID NO:5). Table 15 below describes the starting and ending position of this segment on each transcript.

TABLE 15
Segment location on transcripts

| | Segment | |
|---|---|---|
| | starting | Segment |
| Transcript name | position | ending position |
| T10888_PEA_1_T1 (SEQ ID NO: 3) | 854 | 1108 |
| T10888_PEA_1_T5 (SEQ ID NO: 5) | 854 | 1108 |

Segment cluster T10888_PEA_{—}1_node_{—}12 (SEQ ID NO:101) according to the present invention is supported by 9 libraries. The number of libraries was determined as previously described. This segment can be found in the following transcript(s): T10888_PEA_{—}1_T5 (SEQ ID NO:5). Table 16 below describes the starting and ending position of this segment on each transcript.

TABLE 16
Segment location on transcripts

| | Segment | |
|---|---|---|
| | starting | Segment |
| Transcript name | position | ending position |
| T10888_PEA_1_T5 (SEQ ID NO: 5) | 1109 | 3004 |

Segment cluster T10888_PEA_{—}1_node_{—}17 (SEQ ID NO:102) according to the present invention is supported by 160 libraries. The number of libraries was determined as previously described. This segment can be found in the following transcript(s): T10888_PEA_{—}1_T1 (SEQ ID NO:3) and T10888_PEA_{—}1_T4 (SEQ ID NO:4). Table 17 below describes the starting and ending position of this segment on each transcript.

TABLE 17
Segment location on transcripts

| | Segment | |
|---|---|---|
| | starting | Segment |
| Transcript name | position | ending position |
| T10888_PEA_1_T1 (SEQ ID NO: 3) | 1109 | 2518 |
| T10888_PEA_1_T4 (SEQ ID NO: 4) | 967 | 2376 |

Segment cluster T10888_PEA_{—}1_node_{—}4 (SEQ ID NO:103) according to the present invention is supported by 61 libraries. The number of libraries was determined as previously described. This segment can be found in the following transcript(s): T10888_PEA_{—}1_T1 (SEQ ID NO:3), T10888_PEA_{—}1_T4 (SEQ ID NO:4), T10888_PEA_{—}1_T5 (SEQ ID NO:5) and T10888_PEA_{—}1_T6 (SEQ ID NO:6). Table 18 below describes the starting and ending position of this segment on each transcript.

TABLE 18
Segment location on transcripts

| | Segment | |
|---|---|---|
| | starting | Segment |
| Transcript name | position | ending position |
| T10888_PEA_1_T1 (SEQ ID NO: 3) | 1 | 214 |
| T10888_PEA_1_T4 (SEQ ID NO: 4) | 1 | 214 |
| T10888_PEA_1_T5 (SEQ ID NO: 5) | 1 | 214 |
| T10888_PEA_1_T6 (SEQ ID NO: 6) | 1 | 214 |

Segment cluster T10888_PEA_{—}1_node_{—}6 (SEQ ID NO:104) according to the present invention is supported by 81 libraries. The number of libraries was determined as previously described. This segment can be found in the following transcript(s): T10888_PEA_{—}1_T1 (SEQ ID NO:3), T10888_PEA_{—}1_T4 (SEQ ID NO:4), T10888_PEA_{—}1_T5 (SEQ ID NO:5) and T10888_PEA_{—}1_T6 (SEQ ID NO:6). Table 19 below describes the starting and ending position of this segment on each transcript.

TABLE 19
Segment location on transcripts

| | | Segment |
|---|---|---|
| | Segment | ending |
| Transcript name | starting position | position |
| T10888_PEA_1_T1 (SEQ ID NO: 3) | 215 | 574 |
| T10888_PEA_1_T4 (SEQ ID NO: 4) | 215 | 574 |
| T10888_PEA_1_T5 (SEQ ID NO: 5) | 215 | 574 |
| T10888_PEA_1_T6 (SEQ ID NO: 6) | 215 | 574 |

Segment cluster T10888_PEA_{—}1_node_{—}7 (SEQ ID NO:105) according to the present invention is supported by 4 libraries. The number of libraries was determined as previously described. This segment can be found in the following transcript(s): T10888_PEA_{—}1_T6 (SEQ ID NO:6). Table 20 below describes the starting and ending position of this segment on each transcript.

TABLE 20
Segment location on transcripts

| | | Segment |
|---|---|---|
| | Segment | ending |
| Transcript name | starting position | position |
| T10888_PEA_1_T6 (SEQ ID NO: 6) | 575 | 1410 |

Segment cluster T10888_PEA_{—}1_node_{—}9 (SEQ ID NO:106) according to the present invention is supported by 72 libraries. The number of libraries was determined as previously described. This segment can be found in the following transcript(s): T10888_PEA_{—}1_T1 (SEQ ID NO:3), T10888_PEA_{—}1_T4 (SEQ ID NO:4) and T10888_PEA_{—}1_T5 (SEQ ID NO:5). Table 21 below describes the starting and ending position of this segment on each transcript.

TABLE 21
Segment location on transcripts

| | | Segment |
|---|---|---|
| | Segment | ending |
| Transcript name | starting position | position |
| T10888_PEA_1_T1 (SEQ ID NO: 3) | 575 | 853 |
| T10888_PEA_1_T4 (SEQ ID NO: 4) | 575 | 853 |
| T10888_PEA_1_T5 (SEQ ID NO: 5) | 575 | 853 |

According to an optional embodiment of the present invention, short segments related to the above cluster are also provided. These segments are up to about 120 bp in length, and so are included in a separate description.

Segment cluster T10888_PEA_{—}1_node_{—}15 (SEQ ID NO:107) according to the present invention is supported by 39 libraries. The number of libraries was determined as previously described. This segment can be found in the following transcript(s): T10888_PEA_{—}1_T4 (SEQ ID NO:4). Table 22 below describes the starting and ending position of this segment on each transcript.

TABLE 22
Segment location on transcripts

| | | Segment |
|---|---|---|
| | Segment | ending |
| Transcript name | starting position | position |
| T10888_PEA_1_T4 (SEQ ID NO: 4) | 854 | 966 |

### Variant Protein Alignment to the Previously Known Protein:

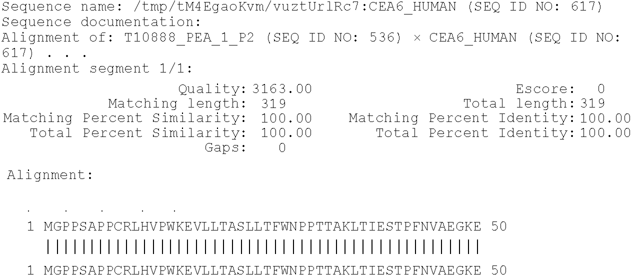

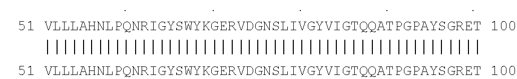

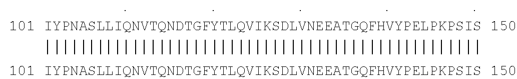

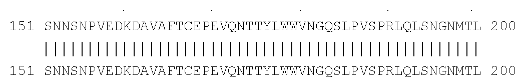

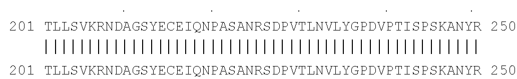

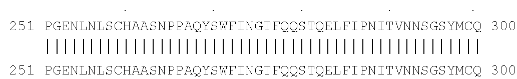

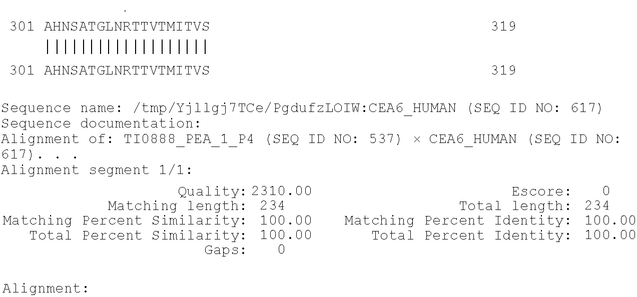

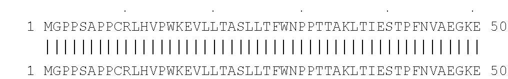

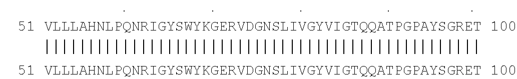

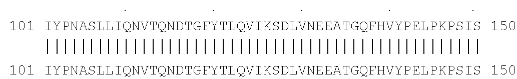

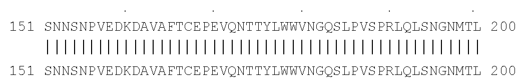

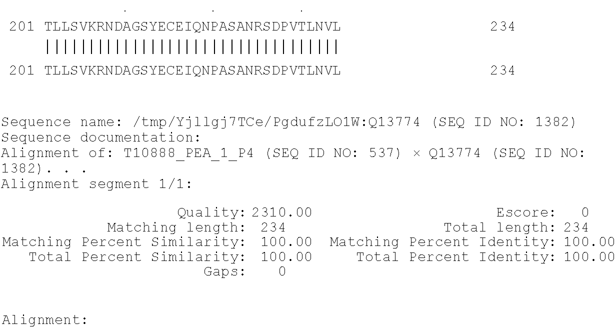

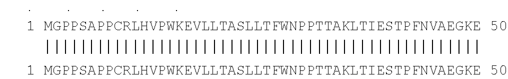

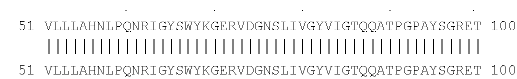

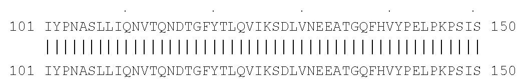

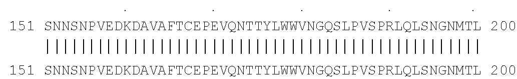

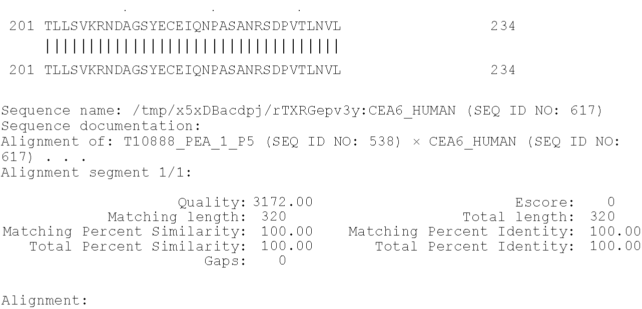

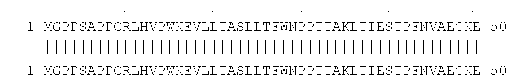

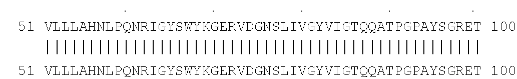

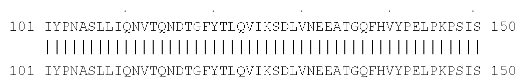

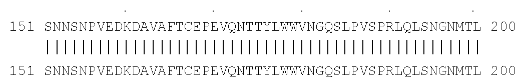

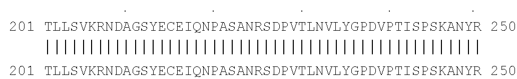

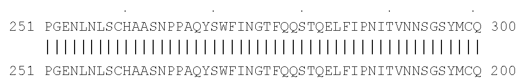

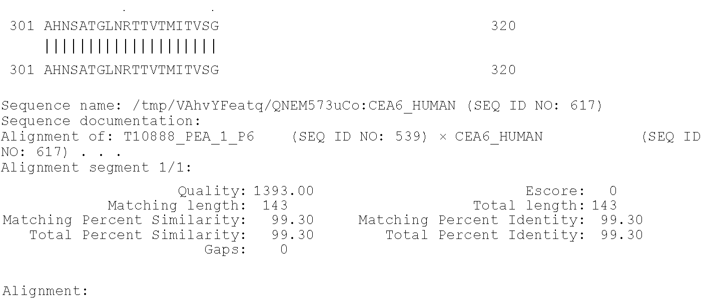

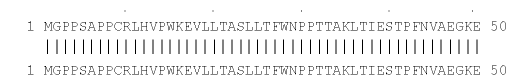

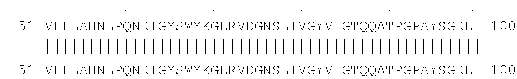

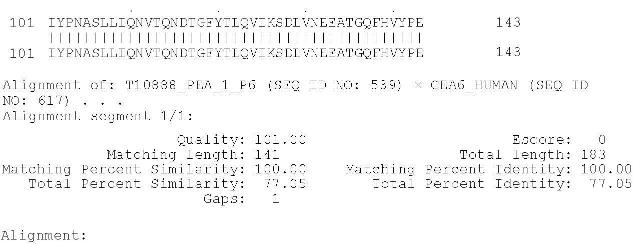

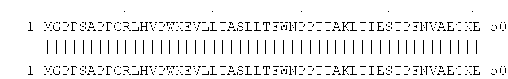

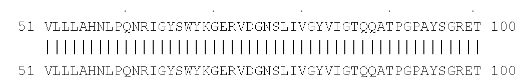

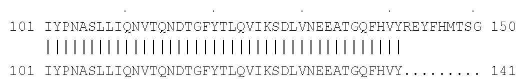

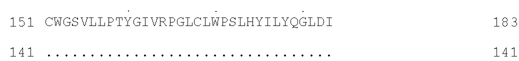

### Expression of CEA6_HUMAN Carcinoembryonic Antigen-Related Cell Adhesion Molecule 6 (T10888)] Transcripts which are Detectable by Amplicon as Depicted in Sequence Name [T10888 junc11-17] (SEQ ID NO: 1279) in Normal and Cancerous Colon Tissues

Expression of CEA6_HUMAN Carcinoembryonic antigen-related cell adhesion molecule 6 transcripts detectable by or according to junc11-17 [node(s)/edge], T10888 junc11-17 amplicon (SEQ ID NO: 1279) and junc11-17 primers (SEQ ID NO: 1277-1278) was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon —HPRT1-amplicon, SEQ ID NO:612), G6PD (GenBank Accession No. NM_{—}000402 (SEQ ID NO:1578); G6PD amplicon, SEQ ID NO:615), and RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, “Tissue samples in testing panel”, above), to obtain a value of fold up-regulation for each sample relative to median of the normal PM samples.

FIG. 10 is a histogram showing over expression of the above-indicated CEA6_HUMAN Carcinoembryonic antigen-related cell adhesion molecule 6 transcripts in cancerous colon samples relative to the normal samples. As is evident from FIG. 10, the expression of CEA6_HUMAN Carcinoembryonic antigen-related cell adhesion molecule 6 transcripts detectable by the above amplicon(s) in cancer samples was significantly higher than in the non-cancerous samples (Sample Nos. 41, 52, 62-67, 69-71, Table 1, “Tissue samples in testing panel”, above). Notably an over-expression of at least 3 fold was found in 15 out of 36 adenocarcinoma samples

Statistical analysis was applied to verify the significance of these results, as described below.

The P value for the difference in the expression levels of CEA6_HUMAN Carcinoembryonic antigen-related cell adhesion molecule 6 transcripts detectable by the above amplicon(s) in colon cancer samples versus the normal tissue samples was determined by T test as 5.36E-03.

Threshold of 3 fold overexpression was found to differentiate between cancer and normal samples with P value of 7.41E-03 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: T10888junc11-17F forward primer (SEQ ID NO: 1277); and T10888junc11-17R reverse primer (SEQ ID NO: 1278).

The present invention also preferably encompasses any amplicon obtained through the use of any suitable primer pair; for example, for the above experiment, the following amplicon was obtained as a non-limiting illustrative example only of a suitable amplicon: T10888junc11-17 (SEQ ID NO: 1279).

Forward:-

(SEQ ID NO: 1277)

CCAGCAATCCACACAAGAGCT
Reverse-

(SEQ ID NO: 1278)

CAGGGTCTGGTCCAATCAGAG
Amplicon-

(SEQ ID NO: 1279)

CCAGCAATCCACACAAGAGCTCTTTATCCCCAACATCACTGTGAATAATA
GCGGATCCTATATGTGCCAAGCCCATAACTCAGCCACTGGCCTCAATAGG
ACCACAGTCACGATGATCACAGTCTCTGATTGGACCAGACCCTG

### Expression of CEA6_HUMAN Carcinoembryonic Antigen-Related Cell Adhesion Molecule 6 T10888 Transcripts, which are Detectable by Amplicon as Depicted in Sequence Name T10888 junc11-17 (SEQ ID NO: 1282) in Different Normal Tissues

Expression of CEA6_HUMAN Carcinoembryonic antigen-related cell adhesion molecule 6 transcripts detectable by or according to T10888 junc11-17 amplicon (SEQ ID NO: 1282) and T10888 junc11-17F (SEQ ID NO: 1280) and T10888 junc11-17R (SEQ ID NO: 1281) was measured by real time PCR. In parallel the expression of four housekeeping genes —RPL19 (GenBank Accession No. NM_{—}000981 (SEQ ID NO:1580); RPL19 amplicon, SEQ ID NO:1264), TATA box (GenBank Accession No. NM_{—}003194 (SEQ ID NO:1581); TATA amplicon, SEQ ID NO:1267), Ubiquitin (GenBank Accession No. BC000449 (SEQ ID NO:1582); amplicon—Ubiquitin-amplicon) and SDHA (GenBank Accession No. NM_{—}004168 (SEQ ID NO:1583); amplicon—SDHA-amplicon, SEQ ID NO:1273) was measured similarly. For each RT sample, the expression of the above amplicon was normalized to the geometric mean of the quantities of the housekeeping genes. The normalized quantity of each RT sample was then divided by the median of the quantities of the ovary samples (Sample Nos. 18-20 Table 2 Tissue samples in normal panel), to obtain a value of relative expression of each sample relative to median of the ovary samples.

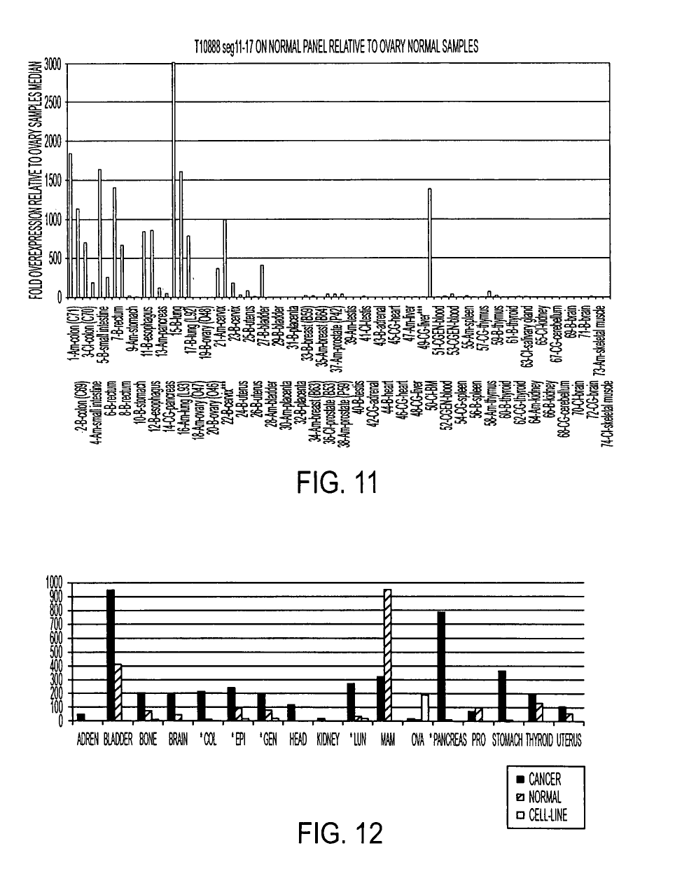

The results are described in FIG. 11, presenting the histogram showing the expression of T10888 transcripts, which are detectable by amplicon as depicted in sequence name T10888junc11-17 (SEQ ID NO: 1282) in different normal tissues.

Forward primer (SEQ ID NO: 1280):
CCAGCAATCCACACAAGAGCT
Reverse primer (SEQ ID NO: 1281):
CAGGGTCTGGTCCAATCAGAG
Amplicon (SEQ ID NO: 1282):
CCAGCAATCCACACAAGAGCTCTTTATCCCCAACATCACTGTGAATAATA
GCGGATCCTATATGTGCCAAGCCCATAACTCAGCCACTGGCCTCAATAGG
ACCACAGTCACGATGATCACAGTCTCTGATTGGACCAGACCCTG

### Description for Cluster H14624

Cluster H14624 features 1 transcript(s) and 15 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| H14624_T20 | 7 |

TABLE 2
Segments of interest

| Segment Name | SEQ ID NO: |
|---|---|
| H14624_node_0 | 108 |
| H14624_node_16 | 109 |
| H14624_node_3 | 110 |
| H14624_node_10 | 111 |
| H14624_node_11 | 112 |
| H14624_node_12 | 113 |
| H14624_node_13 | 114 |
| H14624_node_14 | 115 |
| H14624_node_15 | 116 |
| H14624_node_4 | 117 |
| H14624_node_5 | 118 |
| H14624_node_6 | 119 |
| H14624_node_7 | 120 |
| H14624_node_8 | 121 |
| H14624_node_9 | 122 |

TABLE 3
Proteins of interest

| Protein Name | SEQ ID NO: |
|---|---|
| H14624_P15 | 540 |

Cluster H14624 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 12 and Table 4. This cluster is overexpressed (at least at a minimum level) in the following pathological conditions: colorectal cancer, epithelial malignant tumors, a mixture of malignant tumors from different tissues, lung malignant tumors and pancreas carcinoma.

TABLE 4
Normal tissue distribution

| Name of Tissue | Number |
|---|---|
| adrenal | 0 |
| bladder | 410 |
| bone | 71 |
| brain | 42 |
| colon | 6 |
| epithelial | 91 |
| general | 74 |
| head and neck | 0 |
| kidney | 0 |
| lung | 30 |
| breast | 949 |
| ovary | 7 |
| pancreas | 2 |
| prostate | 94 |
| stomach | 3 |
| Thyroid | 128 |
| uterus | 54 |

TABLE 5
P values and ratios for expression in cancerous tissue

| Name of Tissue | P1 | P2 | SP1 | R3 | SP2 | R4 |
|---|---|---|---|---|---|---|
| adrenal | 4.2e−01 | 4.6^{e}−01 | 4.6e−01 | 2.2 | 5.3e−01 | 1.9 |
| bladder | 5.4e−01 | 6.0e−01 | 1.2e−02 | 1.6 | 2.2e−01 | 1.0 |
| bone | 4.9e−01 | 8.5^{e}−01 | 1.8e−01 | 1.3 | 7.5e−01 | 0.6 |
| brain | 4.7e−01 | 7.0^{e}−01 | 6.3e−05 | 2.3 | 9.4e−03 | 1.4 |
| colon | 4.4e−02 | 9.9^{e}−02 | 4.5e−03 | 5.4 | 2.0e−02 | 3.9 |
| epithelial | 7.7e−03 | 3.6e−01 | 1.5e−11 | 2.0 | 2.9e−02 | 1.1 |
| general | 5.1e−03 | 5.9^{e}−01 | 8.3e−21 | 2.2 | 1.5e−04 | 1.2 |
| head and neck | 1.4e−01 | 2.8^{e}−01 | 4.6e−01 | 2.2 | 7.5e−01 | 1.3 |
| kidney | 6.5e−01 | 7.2^{e}−01 | 5.8e−01 | 1.7 | 7.0e−01 | 1.4 |
| lung | 6.1e−02 | 1.4e−01 | 3.3e−05 | 5.8 | 8.1e−03 | 2.9 |
| breast | 2.4e−01 | 4.1e−01 | 1 | 0.3 | 1 | 0.2 |
| ovary | 8.5e−01 | 7.3^{e}−01 | 6.8e−01 | 1.2 | 1.6e−01 | 1.6 |
| pancreas | 7.5e−03 | 4.9^{e}−02 | 1.2e−21 | 22.4 | 2.4e−16 | 15.1 |
| prostate | 8.3e−01 | 8.9e−01 | 7.2e−01 | 0.8 | 8.8e−01 | 0.6 |
| stomach | 4.6e−01 | 8.5^{e}−01 | 1.0e−03 | 2.7 | 1.1e−01 | 1.4 |
| Thyroid | 7.0e−01 | 7.0^{e}−01 | 5.9e−01 | 1.0 | 5.9e−01 | 1.0 |
| uterus | 4.1e−01 | 7.3^{e}−01 | 2.3e−01 | 1.2 | 6.2e−01 | 0.7 |

As noted above, cluster H14624 features 1 transcript(s), which were listed in Table 1 above. A description of each variant protein according to the present invention is now provided.

Variant protein H14624_P15 (SEQ ID NO:540) according to the present invention has an amino acid sequence as given at the end of the application; it is encoded by transcript(s) H14624_T20 (SEQ ID NO:7). One or more alignments to one or more previously published protein sequences are given at the end of the application. A brief description of the relationship of the variant protein according to the present invention to each such aligned protein is as follows:

Comparison Report Between H14624_P15 (SEQ ID NO:540) and Q9HAP5 (SEQ ID NO:1384) (SEQ ID NO:1384):

1. An isolated chimeric polypeptide encoding for H14624_P15 (SEQ ID NO:540), comprising a first amino acid sequence being at least 90% homologous to MLQGPGSLLLLFLASHCCLGSARGLFLFGQPDFSYKRSNCKPIPANLQLCHGIEYQNMRLPNLLGHETMKE VLEQAGAWIPLVMKQCHPDTKKFLCSLFAPVCLDDLDETIQPCHSLCVQVKDRCAPVMSAFGFPWPDML ECDRFPQDNDLCIPLASSDHLLPATEE corresponding to amino acids 1-167 of Q9HAP5 (SEQ ID NO:1384), which also corresponds to amino acids 1-167 of H14624_P15 (SEQ ID NO:540), and a second amino acid sequence being at least 70%, optionally at least 80%, preferably at least 85%, more preferably at least 90% and most preferably at least 95% homologous to a polypeptide having the sequence GKPSLLLPHSLLG (SEQ ID NO:1486) corresponding to amino acids 168-180 of H14624_P15 (SEQ ID NO:540), wherein said first and second amino acid sequences are contiguous and in a sequential order.

2. An isolated polypeptide encoding for a tail of H14624_P15 (SEQ ID NO:540), comprising a polypeptide being at least 70%, optionally at least about 80%, preferably at least about 85%, more preferably at least about 90% and most preferably at least about 95% homologous to the sequence GKPSLLLPHSLLG (SEQ ID NO:1486) in H14624_P15 (SEQ ID NO:540).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein H14624_P15 (SEQ ID NO:540) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein H14624_P15 (SEQ ID NO:540) sequence provides support for the deduced sequence of this variant protein according to the present invention).

TABLE 6
Amino acid mutations

| SNP position(s) on | Alternative | Previously |
|---|---|---|
| amino acid sequence | amino acid(s) | known SNP? |
| 11 | L -> | No |
| 170 | P -> S | Yes |
| 28 | F -> | No |
| 29 | G -> | No |
| 38 | S -> | No |
| 45 | A -> V | Yes |
| 60 | L -> | No |

Variant protein H14624_P15 (SEQ ID NO:540) is encoded by the following transcript(s): H14624_T20 (SEQ ID NO:7), for which the sequence(s) is/are given at the end of the application. The coding portion of transcript H14624_T20 (SEQ ID NO:7) is shown in bold; this coding portion starts at position 857 and ends at position 1396. The transcript also has the following SNPs as listed in Table 7 (given according to their position on the nucleotide sequence, with the alternative nucleic acid listed; the last column indicates whether the SNP is known or not; the presence of known SNPs in variant protein H14624_P15 (SEQ ID NO:540) sequence provides support for the deduced sequence of this variant protein according to the present invention).

TABLE 7
Nucleic acid SNPs

| SNP position on | Alternative | Previously |
|---|---|---|
| nucleotide sequence | nucleic acid | known SNP? |
| 389 | A -> G | No |
| 476 | C -> T | No |
| 969 | G -> | No |
| 988 | G -> T | Yes |
| 990 | C -> T | Yes |
| 1034 | C -> | No |
| 1168 | C -> T | Yes |
| 1364 | C -> T | Yes |
| 488 | T -> C | No |
| 819 | C -> G | Yes |
| 851 | C -> | No |
| 887 | C -> | No |
| 922 | G -> A | Yes |
| 934 | C -> T | Yes |
| 938 | T -> | No |
| 943 | C -> | No |

As noted above, cluster H14624 features 15 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster H14624_node_{—}0 (SEQ ID NO:108) according to the present invention is supported by 3 libraries. The number of libraries was determined as previously described. This segment can be found in the following transcript(s): H14624_T20 (SEQ ID NO:7). Table 8 below describes the starting and ending position of this segment on each transcript.

TABLE 8
Segment location on transcripts

| | Segment | Segment |
|---|---|---|
| Transcript name | starting position | ending position |
| H14624_T20 (SEQ ID NO: 7) | 1 | 573 |

Segment cluster H14624_node_{—}16 (SEQ ID NO:109) according to the present invention is supported by 3 libraries. The number of libraries was determined as previously described. This segment can be found in the following transcript(s): H14624_T20 (SEQ ID NO:7). Table 9 below describes the starting and ending position of this segment on each transcript.

TABLE 9
Segment location on transcripts

| | Segment | Segment |
|---|---|---|
| Transcript name | starting position | ending position |
| H14624_T20 (SEQ ID NO: 7) | 1359 | 1745 |

Segment cluster H14624_node_{—}3 (SEQ ID NO:110) according to the present invention is supported by 67 libraries. The number of libraries was determined as previously described. This segment can be found in the following transcript(s): H14624_T20 (SEQ ID NO:7). Table 10 below describes the starting and ending position of this segment on each transcript.

TABLE 10
Segment location on transcripts

| | Segment | Segment |
|---|---|---|
| Transcript name | starting position | ending position |
| H14624_T20 (SEQ ID NO: 7) | 574 | 822 |

According to an optional embodiment of the present invention, short segments related to the above cluster are also provided. These segments are up to about 120 bp in length, and so are included in a separate description.

Segment cluster H14624_node_{—}10 (SEQ ID NO:111) according to the present invention can be found in the following transcript(s): H14624_T20 (SEQ ID NO:7). Table 11 below describes the starting and ending position of this segment on each transcript.

TABLE 11
Segment location on transcripts

| | Segment | Segment |
|---|---|---|
| Transcript name | starting position | ending position |
| H14624_T20 (SEQ ID NO: 7) | 1070 | 1079 |

Segment cluster H14624_node_{—}11 (SEQ ID NO:112) according to the present invention is supported by 99 libraries. The number of libraries was determined as previously described. This segment can be found in the following transcript(s): H14624_T20 (SEQ ID NO:7). Table 12 below describes the starting and ending position of this segment on each transcript.

TABLE 12
Segment location on transcripts

| | Segment | Segment |
|---|---|---|
| Transcript name | starting position | ending position |
| H14624_T20 (SEQ ID NO: 7) | 1080 | 1114 |

Segment cluster H14624_node_{—}12 (SEQ ID NO:113) according to the present invention can be found in the following transcript(s): H14624_T20 (SEQ ID NO:7). Table 13 below describes the starting and ending position of this segment on each transcript.

TABLE 13
Segment location on transcripts

| | Segment | Segment |
|---|---|---|
| Transcript name | starting position | ending position |
| H14624_T20 (SEQ ID NO: 7) | 1115 | 1135 |

Segment cluster H14624_node_{—}13 (SEQ ID NO:114) according to the present invention is supported by 124 libraries. The number of libraries was determined as previously described. This segment can be found in the following transcript(s): H14624_T20 (SEQ ID NO:7). Table 14 below describes the starting and ending position of this segment on each transcript.

TABLE 14
Segment location on transcripts

| | Segment | Segment |
|---|---|---|
| Transcript name | starting position | ending position |
| H14624_T20 (SEQ ID NO: 7) | 1136 | 1227 |

Segment cluster H14624_node_{—}14 (SEQ ID NO:115) according to the present invention is supported by 114 libraries. The number of libraries was determined as previously described. This segment can be found in the following transcript(s): H14624_T20 (SEQ ID NO:7). Table 15 below describes the starting and ending position of this segment on each transcript.

TABLE 15
Segment location on transcripts

| | Segment | Segment |
|---|---|---|
| Transcript name | starting position | ending position |
| H14624_T20 (SEQ ID NO: 7) | 1228 | 1287 |

Segment cluster H14624_node_{—}15 (SEQ ID NO:115) according to the present invention is supported by 124 libraries. The number of libraries was determined as previously described. This segment can be found in the following transcript(s): H14624_T20 (SEQ ID NO:7). Table 16 below describes the starting and ending position of this segment on each transcript.

TABLE 16
Segment location on transcripts

| | Segment | Segment |
|---|---|---|
| Transcript name | starting position | ending position |
| H14624_T20 (SEQ ID NO: 7) | 1288 | 1358 |

Segment cluster H14624_node_{—}4 (SEQ ID NO:117) according to the present invention is supported by 65 libraries. The number of libraries was determined as previously described. This segment can be found in the following transcript(s): H14624_T20 (SEQ ID NO:7). Table 17 below describes the starting and ending position of this segment on each transcript.

TABLE 17
Segment location on transcripts

| | Segment | Segment |
|---|---|---|
| Transcript name | starting position | ending position |
| H14624_T20 (SEQ ID NO: 7) | 823 | 892 |

Segment cluster H14624_node_{—}5 (SEQ ID NO:118) according to the present invention can be found in the following transcript(s): H14624_T20 (SEQ ID NO:7). Table 18 below describes the starting and ending position of this segment on each transcript.

TABLE 18
Segment location on transcripts

| | Segment | Segment |
|---|---|---|
| Transcript name | starting position | ending position |
| H14624_T20 (SEQ ID NO: 7) | 893 | 903 |

Segment cluster H14624_node_{—}6 (SEQ ID NO:119) according to the present invention can be found in the following transcript(s): H14624_T20 (SEQ ID NO:7). Table 19 below describes the starting and ending position of this segment on each transcript.

TABLE 19
Segment location on transcripts

| | Segment | Segment |
|---|---|---|
| Transcript name | starting position | ending position |
| H14624_T20 (SEQ ID NO: 7) | 904 | 927 |

Segment cluster H14624_node_{—}7 (SEQ ID NO:120) according to the present invention can be found in the following transcript(s): H14624_T20 (SEQ ID NO:7). Table 20 below describes the starting and ending position of this segment on each transcript.

TABLE 20
Segment location on transcripts

| | Segment | Segment |
|---|---|---|
| Transcript name | starting position | ending position |
| H14624_T20 (SEQ ID NO: 7) | 928 | 934 |

Segment cluster H14624_node_{—}8 (SEQ ID NO:121) according to the present invention is supported by 85 libraries. The number of libraries was determined as previously described. This segment can be found in the following transcript(s): H14624_T20 (SEQ ID NO:7). Table 21 below describes the starting and ending position of this segment on each transcript.

TABLE 21
Segment location on transcripts

| | Segment | Segment |
|---|---|---|
| Transcript name | starting position | ending position |
| H14624_T20 (SEQ ID NO: 7) | 935 | 1014 |

Segment cluster H14624_node_{—}9 (SEQ ID NO:122) according to the present invention is supported by 87 libraries. The number of libraries was determined as previously described. This segment can be found in the following transcript(s): H14624_T20 (SEQ ID NO:7). Table 22 below describes the starting and ending position of this segment on each transcript.

TABLE 22
Segment location on transcripts

| | Segment | Segment |
|---|---|---|
| Transcript name | starting position | ending position |
| H14624_T20 (SEQ ID NO: 7) | 1015 | 1069 |

### Variant Protein Alignment to the Previously Known Protein:

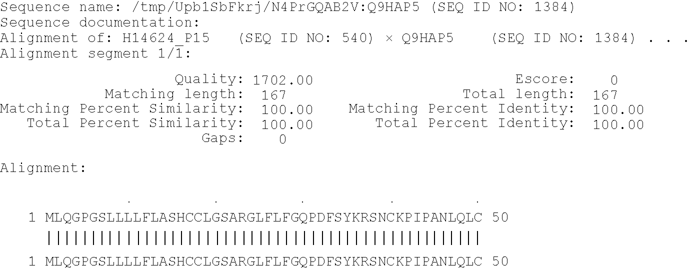

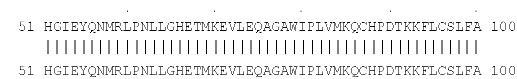

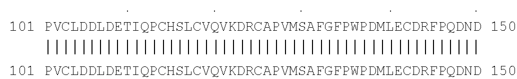

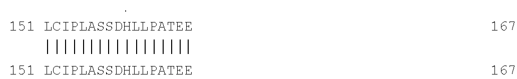

### Description for Cluster H53626

Cluster H53626 features 2 transcript(s) and 20 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| H53626_PEA_1_T15 | 8 |
| H53626_PEA_1_T16 | 9 |

TABLE 2
Segments of interest

| Segment Name | SEQ ID NO: |
|---|---|
| H53626_PEA_1_node_15 | 123 |
| H53626_PEA_1_node_22 | 124 |
| H53626_PEA_1_node_25 | 125 |
| H53626_PEA_1_node_26 | 126 |
| H53626_PEA_1_node_27 | 127 |
| H53626_PEA_1_node_34 | 128 |
| H53626_PEA_1_node_35 | 129 |
| H53626_PEA_1_node_36 | 130 |
| H53626_PEA_1_node_11 | 131 |
| H53626_PEA_1_node_12 | 132 |
| H53626_PEA_1_node_16 | 133 |
| H53626_PEA_1_node_19 | 134 |
| H53626_PEA_1_node_20 | 135 |
| H53626_PEA_1_node_24 | 136 |
| H53626_PEA_1_node_28 | 137 |
| H53626_PEA_1_node_29 | 138 |
| H53626_PEA_1_node_30 | 139 |
| H53626_PEA_1_node_31 | 140 |
| H53626_PEA_1_node_32 | 141 |
| H53626_PEA_1_node_33 | 142 |

TABLE 3
Proteins of interest

| Protein Name | SEQ ID NO: |
|---|---|
| H53626_PEA_1_P4 | 541 |
| H53626_PEA_1_P5 | 542 |

Cluster H53626 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

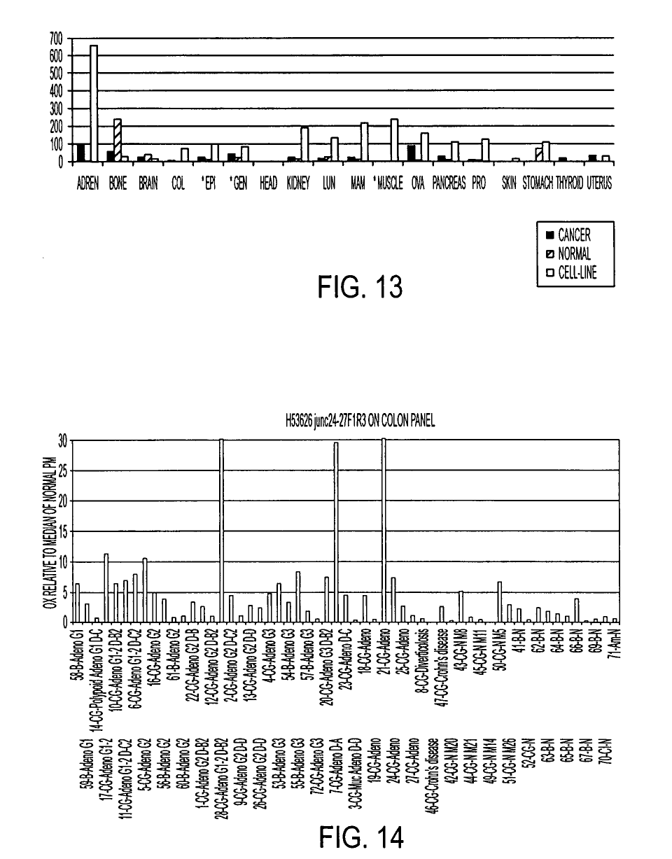

Overall, the following results were obtained as shown with regard to the histograms in FIG. 13 and Table 4. This cluster is overexpressed (at least at a minimum level) in the following pathological conditions: epithelial malignant tumors, a mixture of malignant tumors from different tissues and myosarcoma.

TABLE 4
Normal tissue distribution

| Name of Tissue | Number |
|---|---|
| Adrenal | 4 |
| Bone | 239 |
| Brain | 39 |
| Colon | 0 |
| Epithelial | 12 |
| General | 18 |
| head and neck | 0 |
| Kidney | 8 |
| Lung | 26 |
| Breast | 8 |
| Muscle | 0 |
| Ovary | 7 |
| Pancreas | 10 |
| Prostate | 8 |
| Skin | 0 |
| Stomach | 73 |
| Thyroid | 0 |
| Uterus | 0 |

TABLE 5
P values and ratios for expression in cancerous tissue

| Name of Tissue | P1 | P2 | SP1 | R3 | SP2 | R4 |
|---|---|---|---|---|---|---|
| adrenal | 6.4^{e}−01 | 4.2e−01 | 2.1e−01 | 3.1 | 1.3e−02 | 4.1 |
| bone | 5.8^{e}−01 | 8.1e−01 | 9.8e−01 | 0.3 | 1 | 0.3 |
| Brain | 2.8^{e}−01 | 3.3e−01 | 8.7e−01 | 0.7 | 9.4e−01 | 0.5 |
| Colon | 2.3^{e}−01 | 1.4e−01 | 1 | 1.2 | 4.6e−01 | 1.9 |
| epithelial | 7.2^{e}−02 | 3.7e−03 | 5.8e−02 | 1.6 | 1.4e−08 | 4.3 |
| general | 2.7e−03 | 1.8e−05 | 7.8e−04 | 1.6 | 8.2e−13 | 3.0 |
| Head and neck | 2.1^{e}−01 | 3.3e−01 | 0.0e+00 | 0.0 | 0.0e+00 | 0.0 |
| Kidney | 7.3e−01 | 5.8e−01 | 5.8e−01 | 1.3 | 4.0e−02 | 2.0 |
| lung | 8.4e−01 | 5.8e−01 | 7.9e−01 | 0.8 | 3.7e−02 | 2.0 |
| Breast | 6.5^{e}−01 | 2.7e−01 | 6.9e−01 | 1.2 | 7.8e−02 | 1.9 |
| Muscle | 1 | 2.9e−01 | 1 | 1.0 | 3.5e−03 | 4.1 |
| Ovary | 6.7e−01 | 5.6e−01 | 1.5e−01 | 1.7 | 7.0e−02 | 2.7 |
| pancreas | 2.3e−01 | 2.0e−01 | 3.9e−01 | 1.9 | 8.2e−02 | 2.3 |
| prostate | 9.0^{e}−01 | 9.0e−01 | 6.7e−01 | 1.1 | 1.3e−01 | 1.9 |
| skin | 1 | 4.4e−01 | 1 | 1.0 | 4.1e−01 | 2.1 |
| stomach | 9.0^{e}−01 | 3.4e−01 | 1 | 0.3 | 6.1e−01 | 0.9 |
| Thyroid | 2.4e−01 | 2.4e−01 | 1 | 1.1 | 1 | 1.1 |
| Uterus | 2.1^{e}−01 | 2.4e−01 | 2.9e−01 | 2.5 | 2.6e−01 | 2.2 |

As noted above, cluster H53626 features 2 transcript(s), which were listed in Table 1 above. A description of each variant protein according to the present invention is now provided.

Variant protein H53626_PEA_{—}1_P4 (SEQ ID NO:541) according to the present invention has an amino acid sequence as given at the end of the application; it is encoded by transcript(s) H53626_PEA_{—}1_T15 (SEQ ID NO:8). One or more alignments to one or more previously published protein sequences are given at the end of the application. A brief description of the relationship of the variant protein according to the present invention to each such aligned protein is as follows:

### Comparison Report Between H53626_PEA_{—}1_P4 (SEQ ID NO:541) and Q8N441 (SEQ ID NO:1385):

1. An isolated chimeric polypeptide encoding for H53626_PEA_{—}1_P4 (SEQ ID NO:541), comprising a first amino acid sequence being at least 90% homologous to MTPSPLLLLLLPPLLLGAFPPAAAARGPPKMADKVVPRQVARLGRTVRLQCPVEGDPPPLTMWTKDGRTI HSGWSRFRVLPQGLKVKQVEREDAGVYVCKATNGFGSLSVNYTLVVLDDISPGKESLGPDSSSGGQEDPA SQQWARPRFTQPSKMRRRVIARPVGSSVRLKCVASGHPRPDITWMKDDQALTRPEAAEPRKKKWTLSLK NLRPEDSGKYTCRVSNRAGAINATYKVDVIQRTRSKPVLTGTHPVNTTVDFGGTTSFQCKVRSDVKPVIQ WLKRVEYGAEGRHNSTIDVGGQKFVVLPTGDVWSRPDGSYLNKLLITRARQDDAGMYICLGANTMGYSF RSAFLTVLP corresponding to amino acids 1-357 of Q8N441 (SEQ ID NO:1385), which also corresponds to amino acids 1-357 of H53626_PEA_{—}1_P4 (SEQ ID NO:541), second amino acid sequence being at least 70%, optionally at least 80%, preferably at least 85%, more preferably at least 90% and most preferably at least 95% homologous to a polypeptide having the sequence GARLPRHATPCWCPDPPPGPGVPPTGWGPTLPSRAVLARSSAEGGQPRGTVSTAPGMGLGCSPGLCVGVP LPTSFPLALA (SEQ ID NO:1487) corresponding to amino acids 358-437 of H53626_PEA_{—}1_P4 (SEQ ID NO:541), and a third amino acid sequence being at least 90% homologous to DPKPPGPPVASSSSATSLPWPVVIGIPAGAVFILGTLLLWLCQAQKKPCTPAPAPPLPGHRPPGTARDRSGD KDLPSLAALSAGPGVGLCEEHGSPAAPQHLLGPGPVAGPKLYPKLYTDIHTHTHTHSHTHSHVEGKVHQH IHYQC corresponding to amino acids 358-504 of Q8N441 (SEQ ID NO:1385), which also corresponds to amino acids 438-584 of H53626_PEA_{—}1_P4 (SEQ ID NO:541), wherein said first, second and third amino acid sequences are contiguous and in a sequential order.

2. An isolated polypeptide encoding for an edge portion of H53626_PEA_{—}1_P4 (SEQ ID NO:541) comprising an amino acid sequence being at least 70%, optionally at least about 80%, preferably at least about 85%, more preferably at least about 90% and most preferably at least about 95% homologous to the sequence encoding for GARLPRHATPCWCPDPPPGPGVPPTGWGPTLPSRAVLARSSAEGGQPRGTVSTAPGMGLGCSPGLCVGVP LPTSFPLALA (SEQ ID NO:1487), corresponding to H53626_PEA_{—}1_P4 (SEQ ID NO:541).

The location of the variant protein was determined according to results from a number of different software programs and analyses, including analyses from SignalP and other specialized programs. The variant protein is believed to be located as follows with regard to the cell: membrane. The protein localization is believed to be membrane because although both signal-peptide prediction programs agree that this protein has a signal peptide, both trans-membrane region prediction programs predict that this protein has a trans-membrane region downstream of this signal peptide.

Variant protein H53626_PEA_{—}1_P4 (SEQ ID NO:541) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein H53626_PEA_{—}1_P4 (SEQ ID NO:541) sequence provides support for the deduced sequence of this variant protein according to the present invention).

TABLE 6
Amino acid mutations

| SNP position(s) on amino acid | Alternative | |
|---|---|---|
| sequence | amino acid(s) | Previously known SNP? |
| 193 | R -> L | Yes |
| 300 | G -> | No |
| 319 | Y -> H | No |
| 442 | P -> Q | Yes |
| 504 | R -> L | Yes |
| 521 | G -> | No |
| 544 | P -> L | Yes |
| 573 | E -> G | No |

Variant protein H53626_PEA_{—}1_P4 (SEQ ID NO:541) is encoded by the following transcript(s): H53626_PEA_{—}1_T15 (SEQ ID NO:8), for which the sequence(s) is/are given at the end of the application. The coding portion of transcript H53626_PEA_{—}1_T15 (SEQ ID NO:8) is shown in bold; this coding portion starts at position 17 and ends at position 1768. The transcript also has the following SNPs as listed in Table 7 (given according to their position on the nucleotide sequence, with the alternative nucleic acid listed; the last column indicates whether the SNP is known or not; the presence of known SNPs in variant protein H53626_PEA_{—}1_P4 (SEQ ID NO:541) sequence provides support for the deduced sequence of this variant protein according to the present invention).

TABLE 7
Nucleic acid SNPs

| SNP position on | Alternative | Previously |
|---|---|---|
| nucleotide sequence | nucleic acid | known SNP? |
| 76 | G -> A | Yes |
| 340 | G -> T | No |
| 1647 | C -> T | Yes |
| 1734 | A -> G | No |
| 1797 | G -> | No |
| 1948 | A -> G | Yes |
| 2193 | C -> T | Yes |
| 2308 | C -> T | Yes |
| 2333 | C -> G | Yes |
| 2648 | C -> T | Yes |
| 2649 | G -> A | Yes |
| 2765 | C -> T | Yes |
| 594 | G -> T | Yes |
| 2972 | G -> A | Yes |
| 3027 | C -> G | Yes |
| 907 | T -> C | Yes |
| 916 | C -> | No |
| 971 | T -> C | No |
| 1135 | G -> A | Yes |
| 1341 | C -> A | Yes |
| 1527 | G -> T | Yes |
| 1579 | C -> | No |

Variant protein H53626_PEA_{—}1_P5 (SEQ ID NO:542) according to the present invention has an amino acid sequence as given at the end of the application; it is encoded by transcript(s) H53626_PEA_{—}1_T16 (SEQ ID NO:9). One or more alignments to one or more previously published protein sequences are given at the end of the application. A brief description of the relationship of the variant protein according to the present invention to each such aligned protein is as follows:

Comparison Report Between H53626_PEA_{—}1_P5 (SEQ ID NO:542) and Q9H4D7 (SEQ ID NO:1386):

1. An isolated chimeric polypeptide encoding for H53626_PEA_{—}1_P5 (SEQ ID NO:542), comprising a first amino acid sequence being at least 90% homologous to MTPSPLLLLLLPPLLLGAFPPAAAARGPPKMADKVVPRQVARLGRTVRLQCPVEGDPPPLTMWTKDGRTI HSGWSRFRVLPQGLKVKQVEREDAGVYVCKATNGFGSLSVNYTLVVLDDISPGKESLGPDSSSGGQEDPA SQQWARPRFTQPSKMRRRVIARPVGSSVRLKCVASGHPRPDITWMKDDQALTRPEAAEPRKKKWTLSLK NLRPEDSGKYTCRVSNRAGAINATYKVDVIQRTRSKPVLTGTHPVNTTVDFGGTTSFQCK corresponding to amino acids 1-269 of Q9H4D7 (SEQ ID NO:1386), which also corresponds to amino acids 1-269 of H53626_PEA_{—}1_P5 (SEQ ID NO:542), and a second amino acid sequence being at least 70%, optionally at least 80%, preferably at least 85%, more preferably at least 90% and most preferably at least 95% homologous to a polypeptide having the sequence TQNRQGHLWPPRPRPLACRGPWSSASQPALSSSWAPCSCGFARPRRSRAPPRLPLPCLGTARRGRPATAAE TRTFPRWPPSALALVWGCVRSMGLRQPPSTYWAQAQLLALSCTPNSTQTSTHTHTHTLTHTHTWRARSTS TSTISARRHRICSGHGGAGQTGRLGGWRTELQTKAGDPWRGGMASTPGSLCVRHSPWTHTHRHTHYLDA CMHTHARTRAP (SEQ ID NO:1488) corresponding to amino acids 270-490 of H53626_PEA_{—}1_P5 (SEQ ID NO:542), wherein said first and second amino acid sequences are contiguous and in a sequential order.

2. An isolated polypeptide encoding for a tail of H53626_PEA_{—}1_P5 (SEQ ID NO:542), comprising a polypeptide being at least 70%, optionally at least about 80%, preferably at least about 85%, more preferably at least about 90% and most preferably at least about 95% homologous to the sequence TQNRQGHLWPPRPRPLACRGPWSSASQPALSSSWAPCSCGFARPRRSRAPPRLPLPCLGTARRGRPATAAE TRTFPRWPPSALALVWGCVRSMGLRQPPSTYWAQAQLLALSCTPNSTQTSTHTHTHTLTHTHTWRARSTS TSTISARRHRICSGHGGAGQTGRLGGWRTELQTKAGDPWRGGMASTPGSLCVRHSPWTHTHRHTHYLDA CMHTHARTRAP (SEQ ID NO:1488) in H53626_PEA_{—}1_P5 (SEQ ID NO:542).

Comparison Report Between H53626_PEA_{—}1_P5 (SEQ ID NO:542) and Q8N441 (SEQ ID NO:1385):

1. An isolated chimeric polypeptide encoding for H53626_PEA_{—}1_P5 (SEQ ID NO:542), comprising a first amino acid sequence being at least 90% homologous to MTPSPLLLLLLPPLLLGAFPPAAAARGPPKMADKVVPRQVARLGRTVRLQCPVEGDPPPLTMWTKDGRTI HSGWSRFRVLPQGLKVKQVEREDAGVYVCKATNGFGSLSVNYTLVVLDDISPGKESLGPDSSSGGQEDPA SQQWARPRFTQPSKMRRRVIARPVGSSVRLKCVASGHPRPDITWMKDDQALTRPEAAEPRKKKWTLSLK NLRPEDSGKYTCRVSNRAGAINATYKVDVIQRTRSKPVLTGTHPVNTTVDFGGTTSFQCK corresponding to amino acids 1-269 of Q8N441 (SEQ ID NO:1385), which also corresponds to amino acids 1-269 of H53626_PEA_{—}1_P5 (SEQ ID NO:542), and a second amino acid sequence being at least 70%, optionally at least 80%, preferably at least 85%, more preferably at least 90% and most preferably at least 95% homologous to a polypeptide having the sequence TQNRQGHLWPPRPRPLACRGPWSSASQPALSSSWAPCSCGFARPRRSRAPPRLPLPCLGTARRGRPATAAE TRTFPRWPPSALALVWGCVRSMGLRQPPSTYWAQAQLLALSCTPNSTQTSTHTHTHTLTHTHTWRARSTS TSTISARRHRICSGHGGAGQTGRLGGWRTELQTKAGDPWRGGMASTPGSLCVRHSPWTHTHRHTHYLDA CMHTHARTRAP (SEQ ID NO:1488) corresponding to amino acids 270-490 of H53626_PEA_{—}1_P5 (SEQ ID NO:542), wherein said first and second amino acid sequences are contiguous and in a sequential order.

2. An isolated polypeptide encoding for a tail of H53626_PEA_{—}1_P5 (SEQ ID NO:542), comprising a polypeptide being at least 70%, optionally at least about 80%, preferably at least about 85%, more preferably at least about 90% and most preferably at least about 95% homologous to the sequence TQNRQGHLWPPRPRPLACRGPWSSASQPALSSSWAPCSCGFARPRRSRAPPRLPLPCLGTARRGRPATAAE TRTFPRWPPSALALVWGCVRSMGLRQPPSTYWAQAQLLALSCTPNSTQTSTHTHTHTLTHTHTWRARSTS TSTISARRHRICSGHGGAGQTGRLGGWRTELQTKAGDPWRGGMASTPGSLCVRHSPWTHTHRHTHYLDA CMHTHARTRAP (SEQ ID NO:1488) in H53626_PEA_{—}1_P5 (SEQ ID NO:542).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein H53626_PEA_{—}1_P5 (SEQ ID NO:542) also has the following non-silent SNPs (Single Nucleotide Polymorphisms) as listed in Table 8, (given according to their position(s) on the amino acid sequence, with the alternative amino acid(s) listed; the last column indicates whether the SNP is known or not; the presence of known SNPs in variant protein H53626_PEA_{—}1_P5 (SEQ ID NO:542) sequence provides support for the deduced sequence of this variant protein according to the present invention).

TABLE 8
Amino acid mutations

| SNP position(s) on | Alternative | Previously |
|---|---|---|
| amino acid sequence | amino acid(s) | known SNP? |
| 193 | R -> L | Yes |
| 274 | Q -> K | Yes |
| 336 | A -> S | Yes |
| 353 | A -> | No |
| 376 | Q -> * | Yes |
| 405 | R -> G | No |
| 426 | G -> | No |
| 476 | Y -> C | Yes |

Variant protein H53626_PEA_{—}1_P5 (SEQ ID NO:542) is encoded by the following transcript(s): H53626_PEA_{—}1_T16 (SEQ ID NO:9), for which the sequence(s) is/are given at the end of the application. The coding portion of transcript H53626_PEA_{—}1_T16 (SEQ ID NO:9) is shown in bold; this coding portion starts at position 17 and ends at position 1486. The transcript also has the following SNPs as listed in Table 9 (given according to their position on the nucleotide sequence, with the alternative nucleic acid listed; the last column indicates whether the SNP is known or not; the presence of known SNPs in variant protein H53626_PEA_{—}1_P5 (SEQ ID NO:542) sequence provides support for the deduced sequence of this variant protein according to the present invention).

TABLE 9
Nucleic acid SNPs

| SNP position on | Alternative | Previously |
|---|---|---|
| nucleotide sequence | nucleic acid | known SNP? |
| 76 | G -> A | Yes |
| 340 | G -> T | No |
| 1688 | C -> T | Yes |
| 1803 | C -> T | Yes |
| 1828 | C -> G | Yes |
| 2143 | C -> T | Yes |
| 2144 | G -> A | Yes |
| 2260 | C -> T | Yes |
| 2467 | G -> A | Yes |
| 2522 | C -> G | Yes |
| 594 | G -> T | Yes |
| 836 | C -> A | Yes |
| 1022 | G -> T | Yes |
| 1074 | C -> | No |
| 1142 | C -> T | Yes |
| 1229 | A -> G | No |
| 1292 | G -> | No |
| 1443 | A -> G | Yes |

As noted above, cluster H53626 features 20 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster H53626_PEA_{—}1_node_{—}15 (SEQ ID NO:123) according to the present invention is supported by 25 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 10 below describes the starting and ending position of this segment on each transcript.

TABLE 10
Segment location on transcripts

| | Segment | Segment |
|---|---|---|
| Transcript name | starting position | ending position |
| H53626_PEA_1_T15 (SEQ | 96 | 343 |
| ID NO: 8) | | |
| H53626_PEA_1_T16 (SEQ | 96 | 343 |
| ID NO: 9) | | |

Segment cluster H53626_PEA_{—}1_node_{—}22 (SEQ ID NO:124) according to the present invention is supported by 42 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 11 below describes the starting and ending position of this segment on each transcript.

TABLE 11
Segment location on transcripts

| | Segment | Segment |
|---|---|---|
| Transcript name | starting position | ending position |
| H53626_PEA_1_T15 (SEQ ID | 450 | 734 |
| NO: 8) | | |
| H53626_PEA_1_T16 (SEQ ID | 450 | 734 |
| NO: 9) | | |

Segment cluster H53626_PEA_{—}1_node_{—}25 (SEQ ID NO:125) according to the present invention is supported by 41 libraries. The number of libraries was determined as previously described. This segment can be found in the following transcript(s): H53626_PEA_{—}1_T15 (SEQ ID NO:8). Table 12 below describes the starting and ending position of this segment on each transcript.

TABLE 12
Segment location on transcripts

| | Segment | Segment |
|---|---|---|
| Transcript name | starting position | ending position |
| H53626_PEA_1_T15 (SEQ ID | 824 | 1088 |
| NO: 8) | | |

Segment cluster H53626_PEA_{—}1_node_{—}26 (SEQ ID NO:126) according to the present invention is supported by 5 libraries. The number of libraries was determined as previously described. This segment can be found in the following transcript(s): H53626_PEA_{—}1_T15 (SEQ ID NO:8). Table 14 below describes the starting and ending position of this segment on each transcript.

TABLE 14
Segment location on transcripts

| | Segment | Segment |
|---|---|---|
| Transcript name | starting position | ending position |
| H53626_PEA_1_T15 (SEQ ID | 1089 | 1328 |
| NO: 8) | | |

Microarray (chip) data is also available for this segment as follows. As described above with regard to the cluster itself, various oligonucleotides were tested for being differentially expressed in various disease conditions, particularly cancer. The following oligonucleotides (related to colon cancer) were found to hit this segment, shown in Table 15.

TABLE 15
Oligonucleotides related to this segment

| | Overexpressed | |
|---|---|---|
| Oligonucleotide name | in cancers | Chip reference |
| H53626_0_0_8391 (SEQ ID | colorectal cancer | Colon |
| NO: 1401) | | |

Segment cluster H53626_PEA_{—}1_node_{—}27 (SEQ ID NO:127) according to the present invention is supported by 106 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 16 below describes the starting and ending position of this segment on each transcript.

TABLE 16
Segment location on transcripts

| | Segment | Segment |
|---|---|---|
| Transcript name | starting position | ending position |
| H53626_PEA_1_T15 (SEQ ID | 1329 | 2228 |
| NO: 8) | | |
| H53626_PEA_1_T16 (SEQ ID | 824 | 1723 |
| NO: 9) | | |

Segment cluster H53626_PEA_{—}1_node_{—}34 (SEQ ID NO:128) according to the present invention is supported by 121 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 17 below describes the starting and ending position of this segment on each transcript.

TABLE 17
Segment location on transcripts

| | Segment | Segment |
|---|---|---|
| Transcript name | starting position | ending position |
| H53626_PEA_1_T15 (SEQ ID | 2507 | 2977 |
| NO: 8) | | |
| H53626_PEA_1_T16 (SEQ ID | 2002 | 2472 |
| NO: 9) | | |

Segment cluster H53626_PEA_{—}1_node_{—}35 (SEQ ID NO:129) according to the present invention is supported by 85 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 18 below describes the starting and ending position of this segment on each transcript.

TABLE 18
Segment location on transcripts

| | Segment | Segment |
|---|---|---|
| Transcript name | starting position | ending position |
| H53626_PEA_1_T15 (SEQ ID | 2978 | 3148 |
| NO: 8) | | |
| H53626_PEA_1_T16 (SEQ ID | 2473 | 2643 |
| NO: 9) | | |

Segment cluster H53626_PEA_{—}1_node_{—}36 (SEQ ID NO:130) according to the present invention is supported by 69 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 19 below describes the starting and ending position of this segment on each transcript.

TABLE 19
Segment location on transcripts

| | Segment | Segment |
|---|---|---|
| Transcript name | starting position | ending position |
| H53626_PEA_1_T15 (SEQ ID | 3149 | 3322 |
| NO: 8) | | |
| H53626_PEA_1_T16 (SEQ ID | 2644 | 2817 |
| NO: 9) | | |

According to an optional embodiment of the present invention, short segments related to the above cluster are also provided. These segments are up to about 120 bp in length, and so are included in a separate description.

Segment cluster H53626_PEA_{—}1_node_{—}11 (SEQ ID NO:131) according to the present invention is supported by 12 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 20 below describes the starting and ending position of this segment on each transcript.

TABLE 20
Segment location on transcripts

| | Segment | Segment |
|---|---|---|
| Transcript name | starting position | ending position |
| H53626_PEA_1_T15 (SEQ ID | 1 | 55 |
| NO: 8) | | |
| H53626_PEA_1_T16 (SEQ ID | 1 | 55 |
| NO: 9) | | |

Segment cluster H53626_PEA_{—}1_node_{—}12 (SEQ ID NO:132) according to the present invention is supported by 11 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 21 below describes the starting and ending position of this segment on each transcript.

TABLE 21
Segment location on transcripts

| | Segment | Segment |
|---|---|---|
| Transcript name | starting position | ending position |
| H53626_PEA_1_T15 (SEQ ID | 56 | 95 |
| NO: 8) | | |
| H53626_PEA_1_T16 (SEQ ID | 56 | 95 |
| NO: 9) | | |

Segment cluster H53626_PEA_{—}1_node_{—}16 (SEQ ID NO:133) according to the present invention can be found in the following transcript(s): H53626_PEA_{—}1_T15 (SEQ ID NO:8) and H53626_PEA_{—}1_T16 (SEQ ID NO:9). Table 22 below describes the starting and ending position of this segment on each transcript.

TABLE 22
Segment location on transcripts

| | Segment | Segment |
|---|---|---|
| Transcript name | starting position | ending position |
| H53626_PEA_1_T15 (SEQ ID | 344 | 368 |
| NO: 8) | | |
| H53626_PEA_1_T16 (SEQ ID | 344 | 368 |
| NO: 9) | | |

Segment cluster H53626_PEA_{—}1_node_{—}19 (SEQ ID NO:134) according to the present invention is supported by 25 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 23 below describes the starting and ending position of this segment on each transcript.

TABLE 23
Segment location on transcripts

| | Segment | Segment |
|---|---|---|
| | starting | ending |
| Transcript name | position | position |
| H53626_PEA_1_T15 (SEQ ID NO: 8) | 369 | 419 |
| H53626_PEA_1_T16 (SEQ ID NO: 9) | 369 | 419 |

Segment cluster H53626_PEA_{—}1_node_{—}20 (SEQ ID NO:13b) according to the present invention is supported by 27 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 24 below describes the starting and ending position of this segment on each transcript.

TABLE 24
Segment location on transcripts

| | Segment | Segment |
|---|---|---|
| | starting | ending |
| Transcript name | position | position |
| H53626_PEA_1_T15 (SEQ ID NO: 8) | 420 | 449 |
| H53626_PEA_1_T16 (SEQ ID NO: 9) | 420 | 449 |

Segment cluster H53626_PEA_{—}1_node_{—}24 (SEQ ID NO:136) according to the present invention is supported by 34 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 25 below describes the starting and ending position of this segment on each transcript.

TABLE 25
Segment location on transcripts

| | Segment | Segment |
|---|---|---|
| | starting | ending |
| Transcript name | position | position |
| H53626_PEA_1_T15 (SEQ ID NO: 8) | 735 | 823 |
| H53626_PEA_1_T16 (SEQ ID NO: 9) | 735 | 823 |

Segment cluster H53626_PEA_{—}1_node_{—}28 (SEQ ID NO:137) according to the present invention is supported by 66 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 26 below describes the starting and ending position of this segment on each transcript.

TABLE 26
Segment location on transcripts

| | Segment | Segment |
|---|---|---|
| | starting | ending |
| Transcript name | position | position |
| H53626_PEA_1_T15 (SEQ ID NO: 8) | 2229 | 2306 |
| H53626_PEA_1_T16 (SEQ ID NO: 9) | 1724 | 1801 |

Segment cluster H53626_PEA_{—}1_node_{—}29 (SEQ ID NO:138) according to the present invention is supported by 73 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 27 below describes the starting and ending position of this segment on each transcript.

TABLE 27
Segment location on transcripts

| | Segment | Segment |
|---|---|---|
| | starting | ending |
| Transcript name | position | position |
| H53626_PEA_1_T15 (SEQ ID NO: 8) | 2307 | 2396 |
| H53626_PEA_1_T16 (SEQ ID NO: 9) | 1802 | 1891 |

Segment cluster H53626_PEA_{—}1_node_{—}30 (SEQ ID NO:139) according to the present invention is supported by 71 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 28 below describes the starting and ending position of this segment on each transcript.

TABLE 28
Segment location on transcripts

| | Segment | Segment |
|---|---|---|
| | starting | ending |
| Transcript name | position | position |
| H53626_PEA_1_T15 (SEQ ID NO: 8) | 2397 | 2442 |
| H53626_PEA_1_T16 (SEQ ID NO: 9) | 1892 | 1937 |

Segment cluster H53626_PEA_{—}1_node_{—}31 (SEQ ID NO:140) according to the present invention is supported by 67 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 29 below describes the starting and ending position of this segment on each transcript.

TABLE 29
Segment location on transcripts

| | Segment | Segment |
|---|---|---|
| | starting | ending |
| Transcript name | position | position |
| H53626_PEA_1_T15 (SEQ ID NO: 8) | 2443 | 2469 |
| H53626_PEA_1_T16 (SEQ ID NO: 9) | 1938 | 1964 |

Segment cluster H53626_PEA_{—}1_node_{—}32 (SEQ ID NO:141) according to the present invention is supported by 65 libraries. The number of libraries was determined as previously described. This segment can be found in the following transcript(s): H53626_PEA_{—}1_T15 (SEQ ID NO:8) and H53626_PEA_{—}1_T16 (SEQ ID NO:9). Table 30 below describes the starting and ending position of this segment on each transcript.

TABLE 30
Segment location on transcripts

| | Segment | Segment |
|---|---|---|
| | starting | ending |
| Transcript name | position | position |
| H53626_PEA_1_T15 (SEQ ID NO: 8) | 2470 | 2498 |
| H53626_PEA_1_T16 (SEQ ID NO: 9) | 1965 | 1993 |

Segment cluster H53626_PEA_{—}1_node_{—}33 (SEQ ID NO:142) according to the present invention can be found in the following transcript(s): H53626_PEA_{—}1_T15 (SEQ ID NO:8) and H53626_PEA_{—}1_T16 (SEQ ID NO:9). Table 31 below describes the starting and ending position of this segment on each transcript.

TABLE 31
Segment location on transcripts

| | Segment | Segment |
|---|---|---|
| | starting | ending |
| Transcript name | position | position |
| H53626_PEA_1_T15 (SEQ ID NO: 8) | 2499 | 2506 |
| H53626_PEA_1_T16 (SEQ ID NO: 9) | 1994 | 2001 |

Expression of Homo sapiens Fibroblast Growth Factor Receptor-Like 1 (FGFRL1) H53626 Transcripts, which are Detectable by Amplicon as Depicted in Sequence Name H53626 junc24-27F1R3 (SEQ ID NO: 1285) in Normal and Cancerous Colon Tissues.

Expression of Homo sapiens fibroblast growth factor receptor-like 1 (FGFRL1) transcripts detectable by or according to junc24-27, H53626 junc24-27F1R3 amplicon (SEQ ID NO: 1285) and H53626 junc24-27F1 (SEQ ID NO: 1283) and H53626 junc24-27R3 (SEQ ID NO: 1284) primers was measured by real time PCR. In parallel the expression of four housekeeping genes —PBGD (GenBank Accession No. BC019323 (SEQ ID NO:1576); amplicon —PBGD-amplicon, SEQ ID NO:531), HPRT1 (GenBank Accession No. NM_{—}000194 (SEQ ID NO:1577); amplicon —HPRT1-amplicon, SEQ ID NO:612), and G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 3 above, “Tissue sample in colon cancer testing panel”), to obtain a value of fold up-regulation for each sample relative to median of the normal PM samples.

FIG. 14 is a histogram showing over expression of the above-indicated Homo sapiens fibroblast growth factor receptor-like 1 (FGFRL1) transcripts in cancerous colon samples relative to the normal samples. As is evident from FIG. 14, the expression of Homo sapiens fibroblast growth factor receptor-like 1 (FGFRL1) transcripts detectable by the above amplicon in cancer samples was significantly higher than in the non-cancerous samples (Sample Nos. 41, 52, 62-67, 69-71 Table 3, “Tissue sample in colon cancer testing panel”). Notably an over-expression of at least 5 fold was found in 13 out of 36 adenocarcinoma samples.

Primer pairs are also optionally and preferably encompassed within the present invention; for example, for the above experiment, the following primer pair was used as a non-limiting illustrative example only of a suitable primer pair: H53626 junc24-27F1 forward primer (SEQ ID NO: 1283); and H53626 junc24-27R3 reverse primer (SEQ ID NO: 1284).

The present invention also preferably encompasses any amplicon obtained through the use of any suitable primer pair; for example, for the above experiment, the following amplicon was obtained as a non-limiting illustrative example only of a suitable amplicon: H53626 junc24-27F1R3 (SEQ ID NO: 1285).

Forward primer (SEQ ID NO: 1283):
GTCCTTCCAGTGCAAGACCCA
Reverse primer (SEQ ID NO: 1284):
TGGGCCTGGCAAAGCC
Amplicon (SEQ ID NO: 1285):
GTCCTTCCAGTGCAAGACCCAAAACCGCCAGGGCCACCTGTGGCCTCCTC
GTCCTCGGCCACTAGCCTGCCGTGGCCCGTGGTCATCGGCATCCCAGCCG
GCGCTGTCTTCATCCTGGGCACCCTGCTCCTGTGGCTTTGCCAGGCCCA

Expression of Homo sapiens Fibroblast Growth Factor Receptor-Like 1 (FGFRL1) H53626 Transcripts, which are Detectable by Amplicon as Depicted in Sequence Name H53626 Seg25 (SEQ ID NO: 1288) in Normal and Cancerous Colon Tissues.

Expression of Homo sapiens fibroblast growth factor receptor-like 1 (FGFRL1) transcripts detectable by or according to seg25, H53626 seg25 amplicon (SEQ ID NO: 1288) and H53626 seg25F (SEQ ID NO: 1286) and H53626 seg25R (SEQ ID NO: 1287) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon —HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 3 above, “Tissue samples in colon cancer testing panel”), to obtain a value of fold up-regulation for each sample relative to median of the normal PM samples.

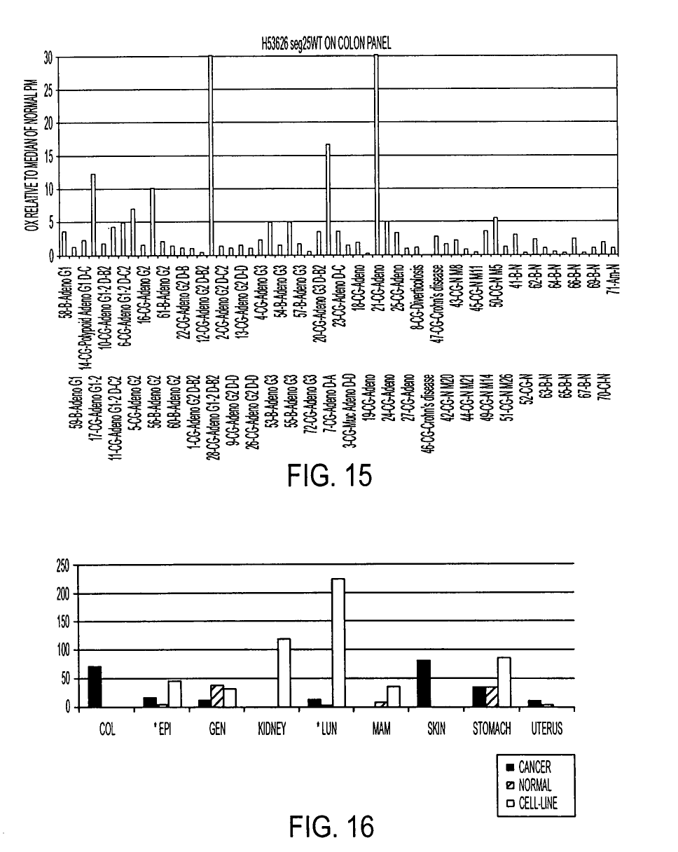

FIG. 15 is a histogram showing over expression of the above-indicated Homo sapiens fibroblast growth factor receptor-like 1 (FGFRL1) transcripts in cancerous colon samples relative to the normal samples. As is evident from FIG. 15, the expression of Homo sapiens fibroblast growth factor receptor-like 1 (FGFRL1) transcripts detectable by the above amplicon was higher in a few cancer samples than in the non-cancerous samples (Sample Nos. 41, 52, 62-67, 69-71 Table 3, “Tissue samples in colon cancer testing panel”). Notably an over-expression of at least 5 fold was found in 6 out of 36 adenocarcinoma samples.

Primer pairs are also optionally and preferably encompassed within the present invention; for example, for the above experiment, the following primer pair was used as a non-limiting illustrative example only of a suitable primer pair: H53626 seg25F forward primer (SEQ ID NO: 1286); and H53626 seg25R reverse primer (SEQ ID NO: 1287).

The present invention also preferably encompasses any amplicon obtained through the use of any suitable primer pair; for example, for the above experiment, the following amplicon was obtained as a non-limiting illustrative example only of a suitable amplicon: H53626 seg25 (SEQ ID NO: 1288).

Forward primer (SEQ ID NO: 1286):
CCGACGGCTCCTACCTCAA
Reverse primer (SEQ ID NO: 1287):
GGAAGCTGTAGCCCATGGTGT
Amplicon (SEQ ID NO: 1288):
CCGACGGCTCCTACCTCAATAAGCTGCTCATCACCCGTGCCCGCCAGGAC
GATGCGGGCATGTACATCTGCCTTGGCGCCAACACCATGGGCTACAGCTT
CC

It should be noted that the variant expression pattern was found to be similar to the expression pattern of the wild-type (previously known) transcript. However, in some cases (as for colon cancer) overexpression of the variant (for example H53626_FGF-RL_T16 transcript) seems to be higher than that the of previously known transcript.

### Expression of Homo sapiens Fibroblast Growth Factor Receptor-Like 1 (FGFRL1) H53626 Transcripts, which are Detectable by Amplicon as Depicted in Sequence Name H53626 Seg25 (SEQ ID NO: 1288) in Different Normal Tissues.

Expression of Homo sapiens fibroblast growth factor receptor-like 1 (FGFRL1) transcripts detectable by or according to H53626 seg25 amplicon (SEQ ID NO: 1288) and H53626 seg25F (SEQ ID NO: 1286) and H53626 seg25R (SEQ ID NO: 1287) was measured by real time PCR. In parallel the expression of four housekeeping genes: RPL19 (GenBank Accession No. NM_{—}000981 (SEQ ID NO:1580); RPL19 amplicon, SEQ ID NO:1264), TATA box (GenBank Accession No. NM_{—}003194 (SEQ ID NO:1581); TATA amplicon, SEQ ID NO:1267), UBC (GenBank Accession No. BC000449 (SEQ ID NO:1582); amplicon—Ubiquitin-amplicon) and SDHA (GenBank Accession No. NM_{—}004168 (SEQ ID NO:1583); amplicon—SDHA-amplicon, SEQ ID NO:1273) was measured similarly. For each RT sample, the expression of the above amplicon was normalized to the geometric mean of the quantities of the housekeeping genes. The normalized quantity of each RT sample was then divided by the median of the quantities of the lung samples (Sample Nos. 15-17 Table 2 above, “Tissue samples in normal panel”), to obtain a value of relative expression of each sample relative to median of the lung samples.

Forward primer (SEQ ID NO: 1286):
CCGACGGCTCCTACCTCAA
Reverse primer (SEQ ID NO: 1287):
GGAAGCTGTAGCCCATGGTGT
Amplicon (SEQ ID NO: 1288):
CCGACGGCTCCTACCTCAATAAGCTGCTCATCACCCGTGCCCGCCAGGAC
GATGCGGGCATGTACATCTGCCTTGGCGCCAACACCATGGGCTACAGCTT
CC

The results are presented in FIG. 71, showing the expression of fibroblast growth factor receptor-like 1 (FGFRL1) transcripts detectable by or according to H53626 seg25 amplicon(s) (SEQ ID NO: 1288) and H53626 seg25F (SEQ ID NO: 1286) and H53626 seg25R (SEQ ID NO: 1287) in different normal tissues.

### Expression of Homo sapiens Fibroblast Growth Factor Receptor-Like 1 (FGFRL1) H53626 Transcripts which are Detectable by Amplicon as Depicted in Sequence Name H53626 junc24-27F1R3 (SEQ ID NO: 1285) in Different Normal Tissues

Expression of Homo sapiens fibroblast growth factor receptor-like 1 (FGFRL1) transcripts detectable by or according to H53626 junc24-27F1R3 amplicon (SEQ ID NO:1285) and H53626 junc24-27F1 (SEQ ID NO:1283) and H53626 junc24-27R3 (SEQ ID NO:1284) was measured by real time PCR. In parallel the expression of four housekeeping genes —RPL19 (GenBank Accession No. NM_{—}000981 (SEQ ID NO:1580); RPL19 amplicon, SEQ ID NO:1264), TATA box (GenBank Accession No. NM_{—}003194 (SEQ ID NO:1581); TATA amplicon, SEQ ID NO:1267), UBC (GenBank Accession No. BC000449 (SEQ ID NO:1582); amplicon-Ubiquitin-amplicon) and SDHA (GenBank Accession No. NM_{—}004168 (SEQ ID NO:1583); amplicon—SDHA-amplicon, SEQ ID NO:1273) was measured similarly. For each RT sample, the expression of the above amplicon was normalized to the geometric mean of the quantities of the housekeeping genes. The normalized quantity of each RT sample was then divided by the median of the quantities of the lung samples (Sample Nos. 15-17 Table 2 above, “Tissue samples in normal panel”), to obtain a value of relative expression of each sample relative to median of the lung samples.

Forward primer (SEQ ID NO: 1283):
GTCCTTCCAGTGCAAGACCCA
Reverse primer (SEQ ID NO: 1284):
TGGGCCTGGCAAAGCC
Amplicon (SEQ ID NO: 1285):
GTCCTTCCAGTGCAAGACCCAAAACCGCCAGGGCCACCTGTGGCCTCCTC
GTCCTCGGCCACTAGCCTGCCGTGGCCCGTGGTCATCGGCATCCCAGCCG
GCGCTGTCTTCATCCTGGGCACCCTGCTCCTGTGGCTTTGCCAGGCCCA

The results are presented in FIG. 72, showing the expression of fibroblast growth factor receptor-like 1 (FGFRL1) transcripts detectable by or according to H53626 seg25 (SEQ ID NO: 1285) amplicon(s) and H53626 seg25F (SEQ ID NO: 1283) and H53626 junc24-27F1R3 (SEQ ID NO: 1284) in different normal tissues.

### Variant Protein Alignment to the Previously Known Protein:

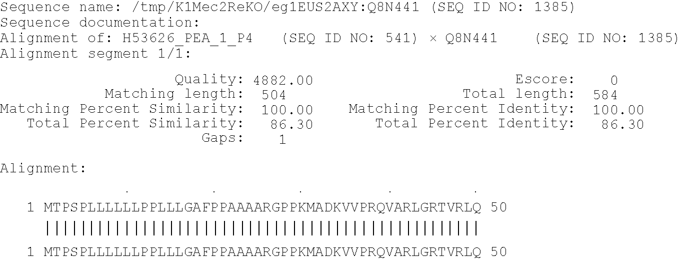

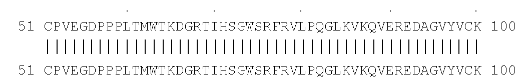

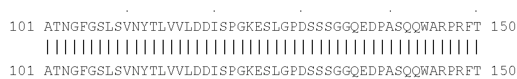

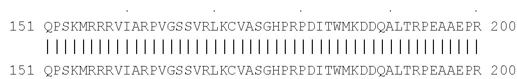

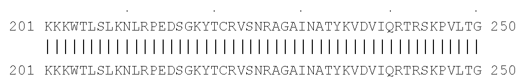

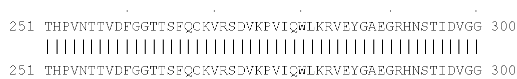

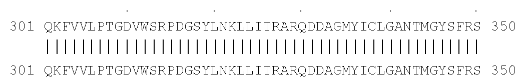

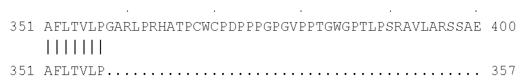

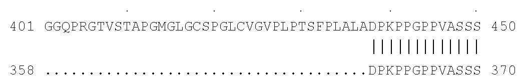

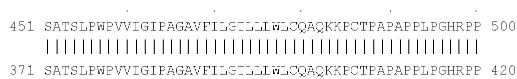

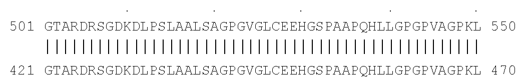

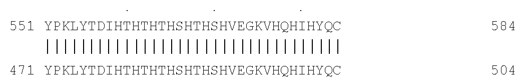

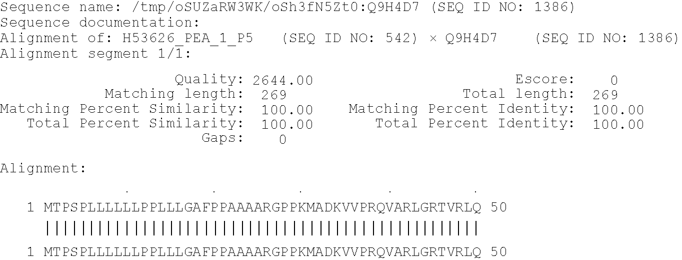

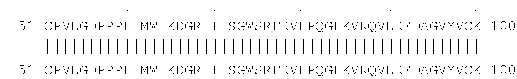

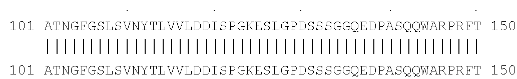

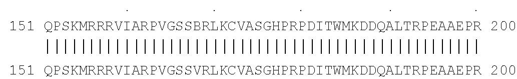

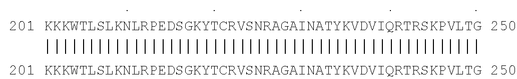

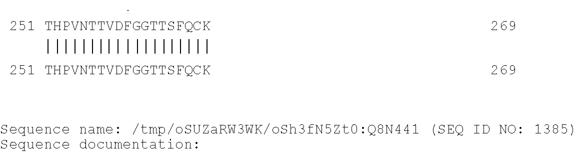

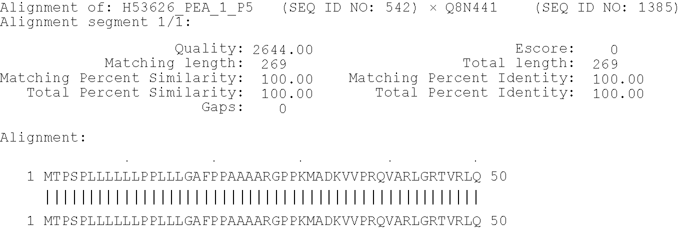

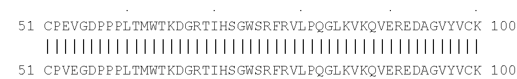

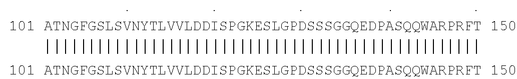

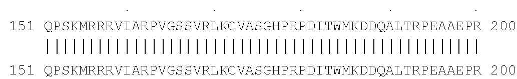

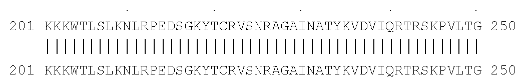

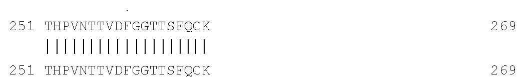

### Description for Cluster HSENA78

Cluster HSENA78 features 1 transcript(s) and 7 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| HSENA78_T5 | 10 |

TABLE 2
Segments of interest

| Segment Name | SEQ ID NO: |
|---|---|
| HSENA78_node_0 | 143 |
| HSENA78_node_2 | 144 |
| HSENA78_node_6 | 145 |
| HSENA78_node_9 | 146 |
| HSENA78_node_3 | 147 |
| HSENA78_node_4 | 148 |
| HSENA78_node_8 | 149 |

TABLE 3
Proteins of interest

| Protein Name | SEQ ID NO: |
|---|---|
| HSENA78_P2 | 543 |

These sequences are variants of the known protein Small inducible cytokine B5 precursor (SwissProt accession identifier SZ05_HUMAN; known also according to the synonyms CXCL5; Epithelial-derived neutrophil activating protein 78; Neutrophil-activating peptide ENA-78), SEQ ID NO: 618, referred to herein as the previously known protein.

Protein Small inducible cytokine B5 precursor (SEQ ID NO:618) is known or believed to have the following function(s): Involved in neutrophil activation. The sequence for protein Small inducible cytokine B5 precursor is given at the end of the application, as “Small inducible cytokine B5 precursor amino acid sequence”. Protein Small inducible cytokine B5 precursor localization is believed to be Secreted.

The following GO Annotation(s) apply to the previously known protein. The following annotation(s) were found: chemotaxis; signal transduction; cell-cell signaling; positive control of cell proliferation, which are annotation(s) related to Biological Process; and chemokine, which are annotation(s) related to Molecular Function.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster HSENA78 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 16 and Table 4. This cluster is overexpressed (at least at a minimum level) in the following pathological conditions: epithelial malignant tumors and lung malignant tumors.

TABLE 4
Normal tissue distribution

| Name of Tissue | Number |
|---|---|
| colon | 0 |
| epithelial | 2 |
| general | 38 |
| kidney | 0 |
| lung | 3 |
| breast | 8 |
| skin | 0 |
| stomach | 36 |
| uterus | 4 |

TABLE 5
P values and ratios for expression in cancerous tissue

| Name of Tissue | P1 | P2 | SP1 | R3 | SP2 | R4 |
|---|---|---|---|---|---|---|
| colon | 2.6e−01 | 3.3e−01 | 1.7e−01 | 2.7 | 2.7e−01 | 2.2 |
| epithelial | 2.5e−01 | 9.0e−02 | 3.2e−03 | 4.1 | 8.5e−07 | 5.5 |
| general | 8.4e−01 | 7.2e−01 | 1 | 0.3 | 1 | 0.4 |
| kidney | 1 | 7.2e−01 | 1 | 1.0 | 1.7e−01 | 1.9 |
| lung | 8.5e−01 | 4.8e−01 | 4.1e−01 | 1.9 | 4.0e−05 | 3.8 |
| breast | 9.5e−01 | 8.7e−01 | 1 | 0.8 | 6.8e−01 | 1.2 |
| skin | 2.9e−01 | 4.7e−01 | 1.4e−01 | 7.0 | 6.4e−01 | 1.6 |
| stomach | 5.0e−01 | 4.3e−01 | 7.5e−01 | 1.0 | 4.3e−01 | 1.3 |
| uterus | 7.1e−01 | 8.5e−01 | 6.6e−01 | 1.3 | 8.0e−01 | 1.0 |

As noted above, cluster HSENA78 features 1 transcript(s), which were listed in Table 1 above. These transcript(s) encode for protein(s) which are variant(s) of protein Small inducible cytokine B5 precursor (SEQ ID NO:618). A description of each variant protein according to the present invention is now provided.

Variant protein HSENA78_P2 (SEQ ID NO:543) according to the present invention has an amino acid sequence as given at the end of the application; it is encoded by transcript(s) HSENA78_T5 (SEQ ID NO:10). An alignment is given to the known protein (Small inducible cytokine B5 precursor (SEQ ID NO:618)) at the end of the application. One or more alignments to one or more previously published protein sequences are given at the end of the application. A brief description of the relationship of the variant protein according to the present invention to each such aligned protein is as follows:

Comparison Report Between HSENA78_P2 (SEQ ID NO:543) and SZ05_HUMAN (SEQ ID NO:618):

1. An isolated chimeric polypeptide encoding for HSENA78_P2 (SEQ ID NO:543), comprising a first amino acid sequence being at least 90% homologous to MSLLSSRAARVPGPSSSLCALLVLLLLLTQPGPIASAGPAAAVLRELRCVCLQTTQGVHPKMISNLQVFAIG PQCSKVEVV corresponding to amino acids 1-81 of SZ05_HUMAN (SEQ ID NO:618), which also corresponds to amino acids 1-81 of HSENA78_P2 (SEQ ID NO:543).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSENA78_P2 (SEQ ID NO:543) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein HSENA78_P2 (SEQ ID NO:543) sequence provides support for the deduced sequence of this variant protein according to the present invention).

TABLE 6
Amino acid mutations

| SNP position(s) on amino acid | Alternative | Previously |
|---|---|---|
| sequence | amino acid(s) | known SNP? |
| 80 | V -> | No |
| 81 | V -> | No |

Variant protein HSENA78_P2 (SEQ ID NO:543) is encoded by the following transcript(s): HSENA78_T5 (SEQ ID NO:10), for which the sequence(s) is/are given at the end of the application. The coding portion of transcript HSENA78_T5 (SEQ ID NO:10) is shown in bold; this coding portion starts at position 149 and ends at position 391. The transcript also has the following SNPs as listed in Table 7 (given according to their position on the nucleotide sequence, with the alternative nucleic acid listed; the last column indicates whether the SNP is known or not; the presence of known SNPs in variant protein HSENA78_P2 (SEQ ID NO:543) sequence provides support for the deduced sequence of this variant protein according to the present invention).

TABLE 7
Nucleic acid SNPs

| SNP position on nucleotide | Alternative | Previously |
|---|---|---|
| sequence | nucleic acid | known SNP? |
| 92 | C -> T | Yes |
| 144 | C -> T | No |
| 1151 | A -> T | Yes |
| 1389 | T -> C | No |
| 1867 | C -> G | Yes |
| 145 | C -> T | No |
| 181 | C -> T | Yes |
| 316 | G -> A | Yes |
| 388 | G -> | No |
| 390 | T -> | No |
| 605 | T -> | No |
| 972 | C -> T | Yes |
| 1105 | A -> G | Yes |

As noted above, cluster HSENA78 features 7 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HSENA78_node_{—}0 (SEQ ID NO:143) according to the present invention is supported by 24 libraries. The number of libraries was determined as previously described. This segment can be found in the following transcript(s): HSENA78_T5 (SEQ ID NO:10). Table 8 below describes the starting and ending position of this segment on each transcript.

TABLE 8
Segment location on transcripts

| | | Segment |
|---|---|---|
| Transcript name | Segment starting position | ending position |
| HSENA78_T5 (SEQ ID | 1 | 257 |
| NO: 10) | | |

Segment cluster HSENA78_node_{—}2 (SEQ ID NO:144) according to the present invention is supported by 22 libraries. The number of libraries was determined as previously described. This segment can be found in the following transcript(s): HSENA78_T5 (SEQ ID NO:10). Table 9 below describes the starting and ending position of this segment on each transcript.

TABLE 9
Segment location on transcripts

| Transcript name | Segment starting position | Segment ending position |
|---|---|---|
| HSENA78_T5 | 258 | 390 |
| (SEQ ID NO: 10) | | |

Segment cluster HSENA78_node_{—}6 (SEQ ID NO:145) according to the present invention is supported by 68 libraries. The number of libraries was determined as previously described. This segment can be found in the following transcript(s): HSENA78_T5 (SEQ ID NO:10). Table 10 below describes the starting and ending position of this segment on each transcript.

TABLE 10
Segment location on transcripts

| | Segment | |
|---|---|---|
| | starting | |
| Transcript name | position | Segment ending position |
| HSENA78_T5 (SEQ ID NO: 10) | 585 | 2370 |

Segment cluster HSENA78_node_{—}9 (SEQ ID NO:146) according to the present invention is supported by 28 libraries. The number of libraries was determined as previously described. This segment can be found in the following transcript(s): HSENA78_T5 (SEQ ID NO:10). Table 11 below describes the starting and ending position of this segment on each transcript.

TABLE 11
Segment location on transcripts

| | Segment | |
|---|---|---|
| | starting | |
| Transcript name | position | Segment ending position |
| HSENA78_T5 (SEQ ID NO: 10) | 2394 | 2546 |

According to an optional embodiment of the present invention, short segments related to the above cluster are also provided. These segments are up to about 120 bp in length, and so are included in a separate description.

Segment cluster HSENA78_node_{—}3 (SEQ ID NO:147) according to the present invention is supported by 1 libraries. The number of libraries was determined as previously described. This segment can be found in the following transcript(s): HSENA78_T5 (SEQ ID NO:10). Table 12 below describes the starting and ending position of this segment on each transcript.

TABLE 12
Segment location on transcripts

| | Segment | |
|---|---|---|
| | starting | |
| Transcript name | position | Segment ending position |
| HSENA78_T5 (SEQ ID NO: 10) | 391 | 500 |

Segment cluster HSENA78_node_{—}4 (SEQ ID NO:148) according to the present invention is supported by 17 libraries. The number of libraries was determined as previously described. This segment can be found in the following transcript(s): HSENA78_T5 (SEQ ID NO:10). Table 13 below describes the starting and ending position of this segment on each transcript.

TABLE 13
Segment location on transcripts

| | Segment | |
|---|---|---|
| | starting | |
| Transcript name | position | Segment ending position |
| HSENA78_T5 (SEQ ID NO: 10) | 501 | 584 |

Segment cluster HSENA78_node_{—}8 (SEQ ID NO:149) according to the present invention can be found in the following transcript(s): HSENA78_T5 (SEQ ID NO:10). Table 14 below describes the starting and ending position of this segment on each transcript.

TABLE 14
Segment location on transcripts

| | Segment | |
|---|---|---|
| | starting | |
| Transcript name | position | Segment ending position |
| HSENA78_T5 (SEQ ID NO: 10) | 2371 | 2393 |

Microarray (chip) data is also available for this gene as follows. As described above with regard to the cluster itself, various oligonucleotides were tested for being differentially expressed in various disease conditions, particularly cancer. The following oligonucleotides were found to hit this segment with regard to colon cancer, shown in Table 15.

TABLE 15
Oligonucleotides related to this gene

| | Overexpressed | Chip |
|---|---|---|
| Oligonucleotide name | in cancers | reference |
| HSENA78_0_1_0 (SEQ ID NO: 1402) | Colon cancer | Colon |

### Variant Protein Alignment to the Previously Known Protein:

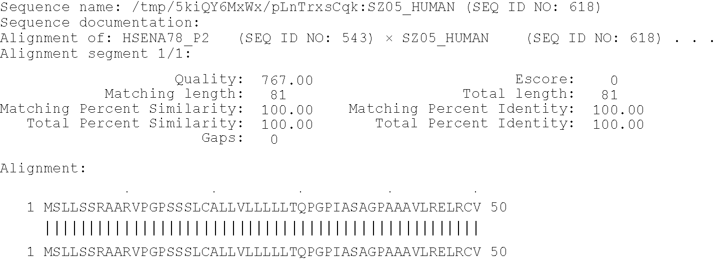

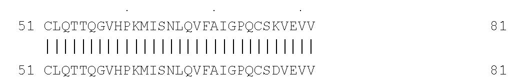

### Description for Cluster HUMGROG5

Cluster HUMGROG5 features 4 transcript(s) and 18 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| HUMGROG5_PEA_1_T3 | 11 |
| HUMGROG5_PEA_1_T4 | 12 |
| HUMGROG5_PEA_1_T6 | 13 |
| HUMGROG5_PEA_1_T9 | 14 |

TABLE 2
Segments of interest

| Segment Name | SEQ ID NO: |
|---|---|
| HUMGROG5_PEA_1_node_18 | 150 |
| HUMGROG5_PEA_1_node_19 | 151 |
| HUMGROG5_PEA_1_node_21 | 152 |
| HUMGROG5_PEA_1_node_23 | 153 |
| HUMGROG5_PEA_1_node_6 | 154 |
| HUMGROG5_PEA_1_node_10 | 155 |
| HUMGROG5_PEA_1_node_11 | 156 |
| HUMGROG5_PEA_1_node_12 | 157 |
| HUMGROG5_PEA_1_node_13 | 158 |
| HUMGROG5_PEA_1_node_14 | 159 |
| HUMGROG5_PEA_1_node_15 | 160 |
| HUMGROG5_PEA_1_node_16 | 161 |
| HUMGROG5_PEA_1_node_17 | 162 |
| HUMGROG5_PEA_1_node_20 | 163 |
| HUMGROG5_PEA_1_node_22 | 164 |
| HUMGROG5_PEA_1_node_7 | 165 |
| HUMGROG5_PEA_1_node_8 | 166 |
| HUMGROG5_PEA_1_node_9 | 167 |

TABLE 3
Proteins of interest

| Protein Name | SEQ ID NO: |
|---|---|
| HUMGROG5_PEA_1_P2 | 544 |
| HUMGROG5_PEA_1_P3 | 545 |
| HUMGROG5_PEA_1_P7 | 546 |
| HUMGROG5_PEA_1_P12 | 547 |

These sequences are variants of the known protein Macrophage inflammatory protein-2-beta precursor (SwissProt accession identifier MI2B_HUMAN; known also according to the synonyms MIP2-beta; CXCL3; Growth regulated protein gamma; GRO-gamma), SEQ ID NO: 619, referred to herein as the previously known protein.

Protein Macrophage inflammatory protein-2-beta precursor (SEQ ID NO:619) is known or believed to have the following function(s): May play a role in inflammation and exert its effects on endothelial cells in an autocrine fashion. The sequence for protein Macrophage inflammatory protein-2-beta precursor is given at the end of the application, as “Macrophage inflammatory protein-2-beta precursor amino acid sequence”. Known polymorphisms for this sequence are as shown in Table 4.

TABLE 4
Amino acid mutations for Known Protein

| SNP position(s) on amino | |
|---|---|
| acid sequence | Comment |
| 27-28 | AA -> G |

Protein Macrophage inflammatory protein-2-beta precursor (SEQ ID NO:619) localization is believed to be Secreted.

The following GO Annotation(s) apply to the previously known protein. The following annotation(s) were found: chemokine, which are annotation(s) related to Molecular Function; and extracellular space, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

As noted above, cluster HUMGROG5 features 4 transcript(s), which were listed in Table 1 above. These transcript(s) encode for protein(s) which are variant(s) of protein Macrophage inflammatory protein-2-beta precursor (SEQ ID NO:619). A description of each variant protein according to the present invention is now provided.

Variant protein HUMGROG5_PEA_{—}1_P2 (SEQ ID NO:544) according to the present invention has an amino acid sequence as given at the end of the application; it is encoded by transcript(s) HUMGROG5_PEA_{—}1_T3 (SEQ ID NO:11). An alignment is given to the known protein (Macrophage inflammatory protein-2-beta precursor (SEQ ID NO:619)) at the end of the application. One or more alignments to one or more previously published protein sequences are given at the end of the application. A brief description of the relationship of the variant protein according to the present invention to each such aligned protein is as follows:

Comparison Report Between HUMGROG5_PEA_{—}1_P2 (SEQ ID NO:544) and MI2B_HUMAN (SEQ ID NO:619):

1. An isolated chimeric polypeptide encoding for HUMGROG5_PEA_{—}1_P2 (SEQ ID NO:544), comprising a first amino acid sequence being at least 90% homologous to MAHATLSAAPSNPRLLRVALLLLLLVAASRRAAGASVVTELRCQCLQTLQGIHLKNIQSVNVRSPGPHCA QTEV corresponding to amino acids 1-74 of MI2B_HUMAN (SEQ ID NO:619), which also corresponds to amino acids 1-74 of HUMGROG5_PEA_{—}1_P2 (SEQ ID NO:544).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMGROG5_PEA_{—}1_P2 (SEQ ID NO:544) also has the following non-silent SNPs (Single Nucleotide Polymorphisms) as listed in Table 5, (given according to their position(s) on the amino acid sequence, with the alternative amino acid(s) listed; the last column indicates whether the SNP is known or not; the presence of known SNPs in variant protein HUMGROG5_PEA_{—}1_P2 (SEQ ID NO:544) sequence provides support for the deduced sequence of this variant protein according to the present invention).

TABLE 5
Amino acid mutations

| SNP position(s) on amino | Alternative | |
|---|---|---|
| acid sequence | amino acid(s) | Previously known SNP? |
| 3 | H -> R | Yes |
| 33 | A -> | No |

Variant protein HUMGROG5_PEA_{—}1_P2 (SEQ ID NO:544) is encoded by the following transcript(s): HUMGROG5_PEA_{—}1_T3 (SEQ ID NO:11), for which the sequence(s) is/are given at the end of the application. The coding portion of transcript HUMGROG5_PEA_{—}1_T3 (SEQ ID NO:11) is shown in bold; this coding portion starts at position 196 and ends at position 420. The transcript also has the following SNPs as listed in Table 6 (given according to their position on the nucleotide sequence, with the alternative nucleic acid listed; the last column indicates whether the SNP is known or not; the presence of known SNPs in variant protein HUMGROG5_PEA_{—}1_P2 (SEQ ID NO:544) sequence provides support for the deduced sequence of this variant protein according to the present invention).

TABLE 6
Nucleic acid SNPs

| SNP position on nucleotide | | Previously |
|---|---|---|
| sequence | Alternative nucleic acid | known SNP? |
| 203 | A -> G | Yes |
| 292 | G -> | No |
| 1062 | A -> G | Yes |
| 1294 | A -> G | Yes |
| 1764 | A -> G | Yes |
| 1901 | A -> T | Yes |

Variant protein HUMGROG5_PEA_{—}1_P3 (SEQ ID NO:545) according to the present invention has an amino acid sequence as given at the end of the application; it is encoded by transcript(s) HUMGROG5_PEA_{—}1_T4 (SEQ ID NO:12). An alignment is given to the known protein (Macrophage inflammatory protein-2-beta precursor (SEQ ID NO:619)) at the end of the application. One or more alignments to one or more previously published protein sequences are given at the end of the application. A brief description of the relationship of the variant protein according to the present invention to each such aligned protein is as follows:

Comparison Report Between HUMGROG5_PEA_{—}1_P3 (SEQ ID NO:545) and MI2B_HUMAN (SEQ ID NO:619):

1. An isolated chimeric polypeptide encoding for HUMGROG5_PEA_{—}1_P3 (SEQ ID NO:545), comprising a first amino acid sequence being at least 90% homologous to MAHATLSAAPSNPRLLRVALLLLLLVAASRRAAGASVVTELRCQCLQTLQGIHLKNIQSVNVRSPGPHCA QTEVIATLKNGKKACLNPASPMVQKIIEKILNK corresponding to amino acids 1-103 of MI2B_HUMAN (SEQ ID NO:619), which also corresponds to amino acids 1-103 of HUMGROG5_PEA_{—}1_P3 (SEQ ID NO:545). The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMGROG5_PEA_{—}1_P3 (SEQ ID NO:545) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein HUMGROG5_PEA_{—}1_P3 (SEQ ID NO:545) sequence provides support for the deduced sequence of this variant protein according to the present invention).

TABLE 7
Amino acid mutations

| SNP position(s) on amino acid | Alternative | |
|---|---|---|
| sequence | amino acid(s) | Previously known SNP? |
| 3 | H -> R | Yes |
| 33 | A -> | No |

Variant protein HUMGROG5_PEA_{—}1_P3 (SEQ ID NO:545) is encoded by the following transcript(s): HUMGROG5_PEA_{—}1_T4 (SEQ ID NO:12), for which the sequence(s) is/are given at the end of the application. The coding portion of transcript HUMGROG5_PEA_{—}1_T4 (SEQ ID NO:12) is shown in bold; this coding portion starts at position 196 and ends at position 504. The transcript also has the following SNPs as listed in Table 8 (given according to their position on the nucleotide sequence, with the alternative nucleic acid listed; the last column indicates whether the SNP is known or not; the presence of known SNPs in variant protein HUMGROG5_PEA_{—}1_P3 (SEQ ID NO:545) sequence provides support for the deduced sequence of this variant protein according to the present invention).

TABLE 8
Nucleic acid SNPs

| SNP position on nucleotide | Alternative | Previously |
|---|---|---|
| sequence | nucleic acid | known SNP? |
| 203 | A -> G | Yes |
| 292 | G -> | No |
| 949 | A -> G | Yes |
| 1181 | A -> G | Yes |
| 1651 | A -> G | Yes |
| 1788 | A -> T | Yes |

Variant protein HUMGROG5_PEA_{—}1_P7 (SEQ ID NO:546) according to the present invention has an amino acid sequence as given at the end of the application; it is encoded by transcript(s) HUMGROG5_PEA_{—}1_T9 (SEQ ID NO:14). An alignment is given to the known protein (Macrophage inflammatory protein-2-beta precursor (SEQ ID NO:619)) at the end of the application. One or more alignments to one or more previously published protein sequences are given at the end of the application. A brief description of the relationship of the variant protein according to the present invention to each such aligned protein is as follows:

Comparison Report Between HUMGROG5_PEA_{—}1_P7 (SEQ ID NO:546) and MI2B_HUMAN (SEQ ID NO:619):

1. An isolated chimeric polypeptide encoding for HUMGROG5_PEA_{—}1_P7 (SEQ ID NO:546), comprising a first amino acid sequence being at least 90% homologous to MAHATLSAAPSNPRLLRVALLLLLLVAASRRAAGASVVTELRCQCLQTLQGIHLKNIQSVN corresponding to amino acids 1-61 of MI2B_HUMAN (SEQ ID NO:619), which also corresponds to amino acids 1-61 of HUMGROG5_PEA_{—}1_P7 (SEQ ID NO:546), and a second amino acid sequence being at least 70%, optionally at least 80%, preferably at least 85%, more preferably at least 90% and most preferably at least 95% homologous to a polypeptide having the sequence SHTQEWEESLSQPRIPHGSENHRKDTEQGEHQLTGEK (SEQ ID NO:1489) corresponding to amino acids 62-98 of HUMGROG5_PEA_{—}1_P7 (SEQ ID NO:546), wherein said first and second amino acid sequences are contiguous and in a sequential order.

2. An isolated polypeptide encoding for a tail of HUMGROG5_PEA_{—}1_P7 (SEQ ID NO:546), comprising a polypeptide being at least 70%, optionally at least about 80%, preferably at least about 85%, more preferably at least about 90% and most preferably at least about 95% homologous to the sequence SHTQEWEESLSQPRIPHGSENHRKDTEQGEHQLTGEK (SEQ ID NO:1489) in HUMGROG5_PEA_{—}1_P7 (SEQ ID NO:546).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMGROG5_PEA_{—}1_P7 (SEQ ID NO:546) also has the following non-silent SNPs (Single Nucleotide Polymorphisms) as listed in Table 9, (given according to their position(s) on the amino acid sequence, with the alternative amino acid(s) listed; the last column indicates whether the SNP is known or not; the presence of known SNPs in variant protein HUMGROG5_PEA_{—}1_P7 (SEQ ID NO:546) sequence provides support for the deduced sequence of this variant protein according to the present invention).

TABLE 9
Amino acid mutations

| SNP position(s) on amino acid | Alternative | |
|---|---|---|
| sequence | amino acid(s) | Previously known SNP? |
| 3 | H -> R | Yes |
| 33 | A -> | No |

Variant protein HUMGROG5_PEA_{—}1_P7 (SEQ ID NO:546) is encoded by the following transcript(s): HUMGROG5_PEA_{—}1_T9 (SEQ ID NO:14), for which the sequence(s) is/are given at the end of the application. The coding portion of transcript HUMGROG5_PEA_{—}1_T9 (SEQ ID NO:14) is shown in bold; this coding portion starts at position 196 and ends at position 489. The transcript also has the following SNPs as listed in Table 10 (given according to their position on the nucleotide sequence, with the alternative nucleic acid listed; the last column indicates whether the SNP is known or not; the presence of known SNPs in variant protein HUMGROG5_PEA_{—}1_P7 (SEQ ID NO:546) sequence provides support for the deduced sequence of this variant protein according to the present invention).

TABLE 10
Nucleic acid SNPs

| SNP position on nucleotide | Alternative | Previously |
|---|---|---|
| sequence | nucleic acid | known SNP? |
| 203 | A -> G | Yes |
| 292 | G -> | No |
| 793 | A -> G | Yes |
| 930 | A -> T | Yes |

Variant protein HUMGROG5_PEA_{—}1_P12 (SEQ ID NO:547) according to the present invention has an amino acid sequence as given at the end of the application; it is encoded by transcript(s) HUMGROG5_PEA_{—}1_T6 (SEQ ID NO:13). An alignment is given to the known protein (Macrophage inflammatory protein-2-beta precursor (SEQ ID NO:619)) at the end of the application. One or more alignments to one or more previously published protein sequences are given at the end of the application. A brief description of the relationship of the variant protein according to the present invention to each such aligned protein is as follows:

Comparison Report Between HUMGROG5_PEA_{—}1_P12 (SEQ ID NO:547) and MI2B_HUMAN (SEQ ID NO:619):

1. An isolated chimeric polypeptide encoding for HUMGROG5_PEA_{—}1_P12 (SEQ ID NO:547) comprising a first amino acid sequence being at least 70%, optionally at least 80%, preferably at least 85%, more preferably at least 90% and most preferably at least 95% homologous to a polypeptide having the sequence MHKKGSPILGSHTARVAGTSPPALPLLAQLPDASAEPHGPRHALRRPQQSPAPAGGAAAPAPGGRQPARS RWVPAPWGPRAGRGWGGRPAPTAPLNQRVYSSL (SEQ ID NO:1490) corresponding to amino acids 1-103 of HUMGROG5_PEA_{—}1_P12 (SEQ ID NO:547), and a second amino acid sequence being at least 90% homologous to GASVVTELRCQCLQTLQGIHLKNIQSVNVRSPGPHCAQTEVIATLKNGKKACLNPASPMVQKIIEKILNKGS TN corresponding to amino acids 34-107 of MI2B_HUMAN (SEQ ID NO:619), which also corresponds to amino acids 104-177 of HUMGROG5_PEA_{—}1_P12 (SEQ ID NO:547), wherein said first and second amino acid sequences are contiguous and in a sequential order.

2. An isolated polypeptide encoding for a head of HUMGROG5_PEA_{—}1_P12 (SEQ ID NO:547) comprising a polypeptide being at least 70%, optionally at least about 80%, preferably at least about 85%, more preferably at least about 90% and most preferably at least about 95% homologous to the sequence MHKKGSPILGSHTARVAGTSPPALPLLAQLPDASAEPHGPRHALRRPQQSPAPAGGAAAPAPGGRQPARS RWVPAPWGPRAGRGWGGRPAPTAPLNQRVYSSL (SEQ ID NO:1490) of HUMGROG5_PEA_{—}1_P12 (SEQ ID NO:547).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein HUMGROG5_PEA_{—}1_P12 (SEQ ID NO:547) also has the following non-silent SNPs (Single Nucleotide Polymorphisms) as listed in Table 11, (given according to their position(s) on the amino acid sequence, with the alternative amino acid(s) listed; the last column indicates whether the SNP is known or not; the presence of known SNPs in variant protein HUMGROG5_PEA_{—}1_P12 (SEQ ID NO:547) sequence provides support for the deduced sequence of this variant protein according to the present invention).

TABLE 11
Amino acid mutations

| SNP position(s) on amino acid | Alternative | Previously |
|---|---|---|
| sequence | amino acid(s) | known SNP? |
| 70 | S -> | No |

Variant protein HUMGROG5_PEA_{—}1_P12 (SEQ ID NO:547) is encoded by the following transcript(s): HUMGROG5_PEA_{—}1_T6 (SEQ ID NO:13), for which the sequence(s) is/are given at the end of the application. The coding portion of transcript HUMGROG5_PEA_{—}1_T6 (SEQ ID NO:13) is shown in bold; this coding portion starts at position 84 and ends at position 614. The transcript also has the following SNPs as listed in Table 12 (given according to their position on the nucleotide sequence, with the alternative nucleic acid listed; the last column indicates whether the SNP is known or not; the presence of known SNPs in variant protein HUMGROG5_PEA_{—}1_P12 (SEQ ID NO:547) sequence provides support for the deduced sequence of this variant protein according to the present invention).

TABLE 12
Nucleic acid SNPs

| SNP position on nucleotide | Alternative | Previously |
|---|---|---|
| sequence | nucleic acid | known SNP? |
| 203 | A -> G | Yes |
| 292 | G -> | No |
| 932 | A -> G | Yes |
| 1069 | A -> T | Yes |

As noted above, cluster HUMGROG5 features 18 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HUMGROG5_PEA_{—}1_node_{—}18 (SEQ ID NO:150) according to the present invention is supported by 23 libraries. The number of libraries was determined as previously described. This segment can be found in the following transcript(s): HUMGROG5_PEA_{—}1_T3 (SEQ ID NO:11) and HUMGROG5_PEA_{—}1_T4 (SEQ ID NO:12). Table 13 below describes the starting and ending position of this segment on each transcript.

TABLE 13
Segment location on transcripts

| | Segment | Segment |
|---|---|---|
| | starting | ending |
| Transcript name | position | position |
| HUMGROG5_PEA_1_T3 (SEQ ID NO: 11) | 617 | 1433 |
| HUMGROG5_PEA_1_T4 (SEQ ID NO: 12) | 504 | 1320 |

Microarray (chip) data is also available for this segment as follows. As described above with regard to the cluster itself, various oligonucleotides were tested for being differentially expressed in various disease conditions, particularly cancer. The following oligonucleotides were found to hit this segment with regard to colon cancer, shown in Table 14.

TABLE 14
Oligonucleotides related to this segment

| Oligonucleotide name | Overexpressed in cancers | Chip reference |
|---|---|---|
| HUMGROG5_0_0_16626 | colorectal cancer | Colon |
| (SEQ ID NO: 1403) | | |

Segment cluster HUMGROG5_PEA_{—}1_node_{—}19 (SEQ ID NO:151) according to the present invention is supported by 40 libraries. The number of libraries was determined as previously described. This segment can be found in the following transcript(s): HUMGROG5_PEA_{—}1_T3 (SEQ ID NO:11), HUMGROG5_PEA_{—}1_T4 (SEQ ID NO:12), HUMGROG5_PEA_{—}1_T6 (SEQ ID NO:13) and HUMGROG5_PEA_{—}1_T9 (SEQ ID NO:14). Table 15 below describes the starting and ending position of this segment on each transcript.

TABLE 15
Segment location on transcripts

| | Segment | Segment |
|---|---|---|
| | starting | ending |
| Transcript name | position | position |
| HUMGROG5_PEA_1_T3 (SEQ ID NO: 11) | 1434 | 1593 |
| HUMGROG5_PEA_1_T4 (SEQ ID NO: 12) | 1321 | 1480 |
| HUMGROG5_PEA_1_T6 (SEQ ID NO: 13) | 602 | 761 |
| HUMGROG5_PEA_1_T9 (SEQ ID NO: 14) | 463 | 622 |

Segment cluster HUMGROG5_PEA_{—}1_node_{—}21 (SEQ ID NO:152) according to the present invention is supported by 45 libraries. The number of libraries was determined as previously described. This segment can be found in the following transcript(s): HUMGROG5_PEA_{—}1_T3 (SEQ ID NO:11), HUMGROG5_PEA_{—}1_T4 (SEQ ID NO:12), HUMGROG5_PEA_{—}1_T6 (SEQ ID NO:13) and HUMGROG5_PEA_{—}1_T9 (SEQ ID NO:14). Table 16 below describes the starting and ending position of this segment on each transcript.

TABLE 16
Segment location on transcripts

| | Segment | Segment |
|---|---|---|
| | starting | ending |
| Transcript name | position | position |
| HUMGROG5_PEA_1_T3 (SEQ ID NO: 11) | 1607 | 1782 |
| HUMGROG5_PEA_1_T4 (SEQ ID NO: 12) | 1494 | 1669 |
| HUMGROG5_PEA_1_T6 (SEQ ID NO: 13) | 775 | 950 |
| HUMGROG5_PEA_1_T9 (SEQ ID NO: 14) | 636 | 811 |

Segment cluster HUMGROG5_PEA_{—}1_node_{—}23 (SEQ ID NO:153) according to the present invention is supported by 60 libraries. The number of libraries was determined as previously described. This segment can be found in the following transcript(s): HUMGROG5_PEA_{—}1_T3 (SEQ ID NO:11), HUMGROG5_PEA_{—}1_T4 (SEQ ID NO:12), HUMGROG5_PEA_{—}1_T6 (SEQ ID NO:13) and HUMGROG5_PEA_{—}1_T9 (SEQ ID NO:14). Table 17 below describes the starting and ending position of this segment on each transcript.

TABLE 17
Segment location on transcripts

| | Segment | Segment |
|---|---|---|
| | starting | ending |
| Transcript name | position | position |
| HUMGROG5_PEA_1_T3 (SEQ ID NO: 11) | 1796 | 2131 |
| HUMGROG5_PEA_1_T4 (SEQ ID NO: 12) | 1683 | 2018 |
| HUMGROG5_PEA_1_T6 (SEQ ID NO: 13) | 964 | 1299 |
| HUMGROG5_PEA_1_T9 (SEQ ID NO: 14) | 825 | 1160 |

Segment cluster HUMGROG5_PEA_{—}1_node_{—}6 (SEQ ID NO:154) according to the present invention is supported by 22 libraries. The number of libraries was determined as previously described. This segment can be found in the following transcript(s): HUMGROG5_PEA_{—}1_T3 (SEQ ID NO:11), HUMGROG5_PEA_{—}1_T4 (SEQ ID NO:12), HUMGROG5_PEA_{—}1_T6 (SEQ ID NO:13) and HUMGROG5_PEA_{—}1_T9 (SEQ ID NO:14). Table 18 below describes the starting and ending position of this segment on each transcript.

TABLE 18
Segment location on transcripts

| | Segment | Segment |
|---|---|---|
| | starting | ending |
| Transcript name | position | position |
| HUMGROG5_PEA_1_T3 (SEQ ID NO: 11) | 1 | 222 |
| HUMGROG5_PEA_1_T4 (SEQ ID NO: 12) | 1 | 222 |
| HUMGROG5_PEA_1_T6 (SEQ ID NO: 13) | 1 | 222 |
| HUMGROG5_PEA_1_T9 (SEQ ID NO: 14) | 1 | 222 |

According to an optional embodiment of the present invention, short segments related to the above cluster are also provided. These segments are up to about 120 bp in length, and so are included in a separate description.

Segment cluster HUMGROG5_PEA_{—}1_node_{—}10 (SEQ ID NO:155) according to the present invention can be found in the following transcript(s): HUMGROG5_PEA_{—}1_T3 (SEQ ID NO:11), HUMGROG5_PEA_{—}1_T4 (SEQ ID NO:12), HUMGROG5_PEA_{—}1_T6 (SEQ ID NO:13) and HUMGROG5_PEA_{—}1_T9 (SEQ ID NO:14). Table 19 below describes the starting and ending position of this segment on each transcript.

TABLE 19
Segment location on transcripts

| | Segment | Segment |
|---|---|---|
| | starting | ending |
| Transcript name | position | position |
| HUMGROG5_PEA_1_T3 (SEQ ID NO: 11) | 296 | 315 |
| HUMGROG5_PEA_1_T4 (SEQ ID NO: 12) | 296 | 315 |
| HUMGROG5_PEA_1_T6 (SEQ ID NO: 13) | 394 | 413 |
| HUMGROG5_PEA_1_T9 (SEQ ID NO: 14) | 296 | 315 |

Segment cluster HUMGROG5_PEA_{—}1_node_{—}11 (SEQ ID NO:156) according to the present invention is supported by 33 libraries. The number of libraries was determined as previously described. This segment can be found in the following transcript(s): HUMGROG5_PEA_{—}1_T3 (SEQ ID NO:11), HUMGROG5_PEA_{—}1_T4 (SEQ ID NO:12), HUMGROG5_PEA_{—}1_T6 (SEQ ID NO:13) and HUMGROG5_PEA_{—}1_T9 (SEQ ID NO:14). Table 20 below describes the starting and ending position of this segment on each transcript.

TABLE 20
Segment location on transcripts

| | Segment | Segment |
|---|---|---|
| | starting | ending |
| Transcript name | position | position |
| HUMGROG5_PEA_1_T3 (SEQ ID NO: 11) | 316 | 378 |
| HUMGROG5_PEA_1_T4 (SEQ ID NO: 12) | 316 | 378 |
| HUMGROG5_PEA_1_T6 (SEQ ID NO: 13) | 414 | 476 |
| HUMGROG5_PEA_1_T9 (SEQ ID NO: 14) | 316 | 378 |

Segment cluster HUMGROG5_PEA_{—}1_node_{—}12 (SEQ ID NO:157) according to the present invention can be found in the following transcript(s): HUMGROG5_PEA_{—}1_T3 (SEQ ID NO:11), HUMGROG5_PEA_{—}1_T4 (SEQ ID NO:12) and HUMGROG5_PEA_{—}1_T6 (SEQ ID NO:13). Table 21 below describes the starting and ending position of this segment on each transcript.

TABLE 21
Segment location on transcripts

| | Segment | Segment |
|---|---|---|
| | starting | ending |
| Transcript name | position | position |
| HUMGROG5_PEA_1_T3 (SEQ ID NO: 11) | 379 | 399 |
| HUMGROG5_PEA_1_T4 (SEQ ID NO: 12) | 379 | 399 |
| HUMGROG5_PEA_1_T6 (SEQ ID NO: 13) | 477 | 497 |

Segment cluster HUMGROG5_PEA_{—}1_node_{—}13 (SEQ ID NO:158) according to the present invention can be found in the following transcript(s): HUMGROG5_PEA_{—}1_T3 (SEQ ID NO:11), HUMGROG5_PEA_{—}1_T4 (SEQ ID NO:12) and HUMGROG5_PEA_{—}1_T6 (SEQ ID NO:13). Table 22 below describes the starting and ending position of this segment on each transcript.

TABLE 22
Segment location on transcripts

| | Segment | Segment |
|---|---|---|
| | starting | ending |
| Transcript name | position | position |
| HUMGROG5_PEA_1_T3 (SEQ ID NO: 11) | 400 | 419 |
| HUMGROG5_PEA_1_T4 (SEQ ID NO: 12) | 400 | 419 |
| HUMGROG5_PEA_1_T6 (SEQ ID NO: 13) | 498 | 517 |

Segment cluster HUMGROG5_PEA_{—}1_node_{—}14 (SEQ ID NO:159) according to the present invention is supported by 8 libraries. The number of libraries was determined as previously described. This segment can be found in the following transcript(s): HUMGROG5_PEA_{—}1_T3 (SEQ ID NO:11). Table 23 below describes the starting and ending position of this segment on each transcript.

TABLE 23
Segment location on transcripts

| | Segment | Segment |
|---|---|---|
| | starting | ending |
| Transcript name | position | position |
| HUMGROG5_PEA_1_T3 (SEQ ID NO: 11) | 420 | 451 |

Segment cluster HUMGROG5_PEA_{—}1_node_{—}15 (SEQ ID NO:160) according to the present invention is supported by 7 libraries. The number of libraries was determined as previously described. This segment can be found in the following transcript(s): HUMGROG5_PEA_{—}1_T3 (SEQ ID NO:11). Table 24 below describes the starting and ending position of this segment on each transcript.

TABLE 24
Segment location on transcripts

| | Segment | Segment |
|---|---|---|
| | starting | ending |
| Transcript name | position | position |
| HUMGROG5_PEA_1_T3 (SEQ ID NO: 11) | 452 | 499 |

Segment cluster HUMGROG5_PEA_{—}1_node_{—}16 (SEQ ID NO:161) according to the present invention is supported by 6 libraries. The number of libraries was determined as previously described. This segment can be found in the following transcript(s): HUMGROG5_PEA_{—}1_T3 (SEQ ID NO:11). Table. 25 below describes the starting and ending position of this segment on each transcript.

TABLE 25
Segment location on transcripts

| | Segment | Segment |
|---|---|---|
| | starting | ending |
| Transcript name | position | position |
| HUMGROG5_PEA_1_T3 (SEQ ID NO: 11) | 500 | 532 |

Segment cluster HUMGROG5_PEA_{—}1_node_{—}17 (SEQ ID NO:162) according to the present invention is supported by 29 libraries. The number of libraries was determined as previously described. This segment can be found in the following transcript(s): HUMGROG5_PEA_{—}1_T3 (SEQ ID NO:11), HUMGROG5_PEA_{—}1_T4 (SEQ ID NO:12), HUMGROG5_PEA_{—}1_T6 (SEQ ID NO:13) and HUMGROG5_PEA_{—}1_T9 (SEQ ID NO:14). Table 26 below describes the starting and ending position of this segment on each transcript.

TABLE 26
Segment location on transcripts

| | Segment | Segment |
|---|---|---|
| | starting | ending |
| Transcript name | position | position |
| HUMGROG5_PEA_1_T3 (SEQ ID NO: 11) | 533 | 616 |
| HUMGROG5_PEA_1_T4 (SEQ ID NO: 12) | 420 | 503 |
| HUMGROG5_PEA_1_T6 (SEQ ID NO: 13) | 518 | 601 |
| HUMGROG5_PEA_1_T9 (SEQ ID NO: 14) | 379 | 462 |

Segment cluster HUMGROG5_PEA_{—}1_node_{—}20 (SEQ ID NO:163) according to the present invention can be found in the following transcript(s): HUMGROG5_PEA_{—}1_T3 (SEQ ID NO:11), HUMGROG5_PEA_{—}1_T4 (SEQ ID NO:12), HUMGROG5_PEA_{—}1_T6 (SEQ ID NO:13) and HUMGROG5_PEA_{—}1_T9 (SEQ ID NO:14). Table 27 below describes the starting and ending position of this segment on each transcript.

TABLE 27
Segment location on transcripts

| | Segment | Segment |
|---|---|---|
| | starting | ending |
| Transcript name | position | position |
| HUMGROG5_PEA_1_T3 (SEQ ID NO: 11) | 1594 | 1606 |
| HUMGROG5_PEA_1_T4 (SEQ ID NO: 12) | 1481 | 1493 |
| HUMGROG5_PEA_1_T6 (SEQ ID NO: 13) | 762 | 774 |
| HUMGROG5_PEA_1_T9 (SEQ ID NO: 14) | 623 | 635 |

Segment cluster HUMGROG5_PEA_{—}1_node_{—}22 (SEQ ID NO:164) according to the present invention can be found in the following transcript(s): HUMGROG5_PEA_{—}1_T3 (SEQ ID NO:11), HUMGROG5_PEA_{—}1_T4 (SEQ ID NO:12), HUMGROG5_PEA_{—}1_T6 (SEQ ID NO:13) and HUMGROG5_PEA_{—}1_T9 (SEQ ID NO:14). Table 28 below describes the starting and ending position of this segment on each transcript.

TABLE 28
Segment location on transcripts

| | Segment | Segment |
|---|---|---|
| | starting | ending |
| Transcript name | position | position |
| HUMGROG5_PEA_1_T3 (SEQ ID NO: 11) | 1783 | 1795 |
| HUMGROG5_PEA_1_T4 (SEQ ID NO: 12) | 1670 | 1682 |
| HUMGROG5_PEA_1_T6 (SEQ ID NO: 13) | 951 | 963 |
| HUMGROG5_PEA_1_T9 (SEQ ID NO: 14) | 812 | 824 |

Segment cluster HUMGROG5_PEA_{—}1_node_{—}7 (SEQ ID NO:165) according to the present invention is supported by 23 libraries. The number of libraries was determined as previously described. This segment can be found in the following transcript(s): HUMGROG5_PEA_{—}1_T3 (SEQ ID NO:11), HUMGROG5_PEA_{—}1_T4 (SEQ ID NO:12), HUMGROG5_PEA_{—}1_T6 (SEQ ID NO:13) and HUMGROG5_PEA_{—}1_T9 (SEQ ID NO:14). Table 29 below describes the starting and ending position of this segment on each transcript.

TABLE 29
Segment location on transcripts

| | Segment | Segment |
|---|---|---|
| | starting | ending |
| Transcript name | position | position |
| HUMGROG5_PEA_1_T3 (SEQ ID NO: 11) | 223 | 270 |
| HUMGROG5_PEA_1_T4 (SEQ ID NO: 12) | 223 | 270 |
| HUMGROG5_PEA_1_T6 (SEQ ID NO: 13) | 223 | 270 |
| HUMGROG5_PEA_1_T9 (SEQ ID NO: 14) | 223 | 270 |

Segment cluster HUMGROG5_PEA_{—}1_node_{—}8 (SEQ ID NO:166) according to the present invention can be found in the following transcript(s): HUMGROG5_PEA_{—}1_T3 (SEQ ID NO:11), HUMGROG5_PEA_{—}1_T4 (SEQ ID NO:12), HUMGROG5_PEA_{—}1_T6 (SEQ ID NO:13) and HUMGROG5_PEA_{—}1_T9 (SEQ ID NO:14). Table 30 below describes the starting and ending position of this segment on each transcript.

TABLE 30
Segment location on transcripts

| | Segment | Segment |
|---|---|---|
| | starting | ending |
| Transcript name | position | position |
| HUMGROG5_PEA_1_T3 (SEQ ID NO: 11) | 271 | 295 |
| HUMGROG5_PEA_1_T4 (SEQ ID NO: 12) | 271 | 295 |
| HUMGROG5_PEA_1_T6 (SEQ ID NO: 13) | 271 | 295 |
| HUMGROG5_PEA_1_T9 (SEQ ID NO: 14) | 271 | 295 |

Segment cluster HUMGROG5_PEA_{—}1_node_{—}9 (SEQ ID NO:167) according to the present invention is supported by 3 libraries. The number of libraries was determined as previously described. This segment can be found in the following transcript(s): HUMGROG5_PEA_{—}1_T6 (SEQ ID NO:13). Table 31 below describes the starting and ending position of this segment on each transcript.

TABLE 31
Segment location on transcripts

| | Segment | Segment |
|---|---|---|
| Transcript name | starting position | ending position |
| HUMGROG5_PEA_1_T6 (SEQ ID | 296 | 393 |
| NO: 13) | | |

### Variant Protein Alignment to the Previously Known Protein:

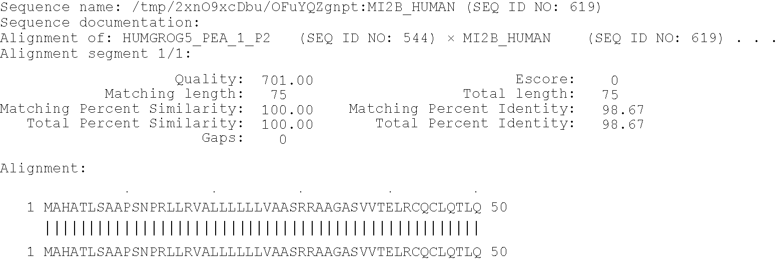

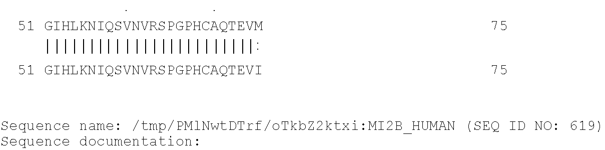

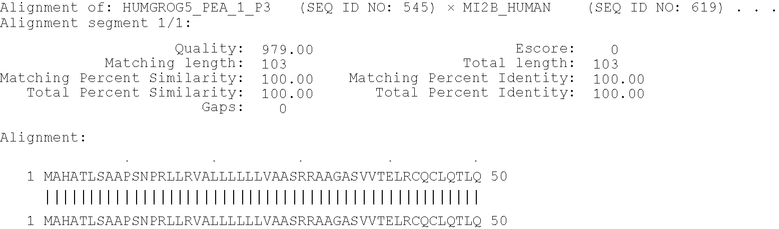

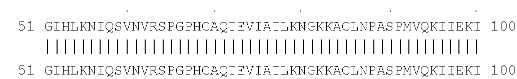

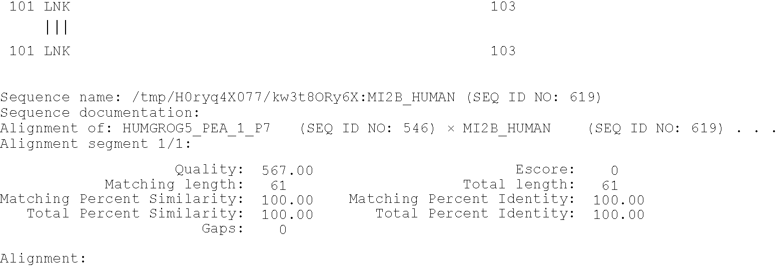

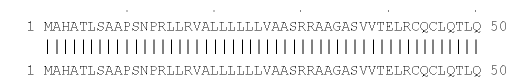

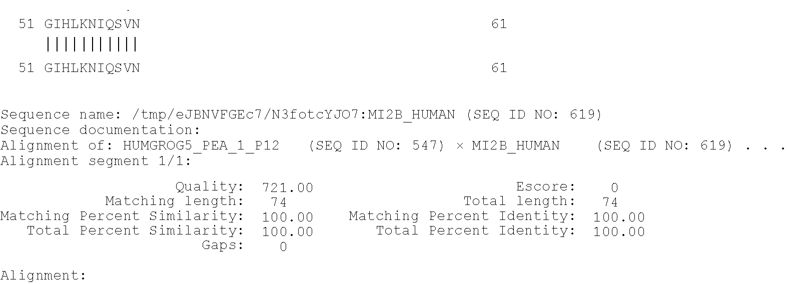

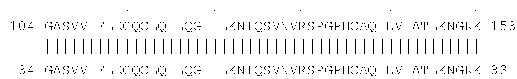

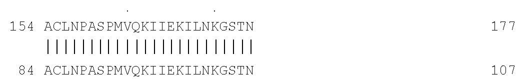

### Description for Cluster HUMODCA

Cluster HUMODCA features 1 transcript(s) and 17 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| HUMODCA_T17 | 15 |

TABLE 2
Segments of interest

| Segment Name | SEQ ID NO: |
|---|---|
| HUMODCA_node_1 | 168 |
| HUMODCA_node_25 | 169 |
| HUMODCA_node_32 | 170 |
| HUMODCA_node_36 | 171 |
| HUMODCA_node_39 | 172 |
| HUMODCA_node_41 | 173 |
| HUMODCA_node_0 | 174 |
| HUMODCA_node_10 | 175 |
| HUMODCA_node_12 | 176 |
| HUMODCA_node_13 | 177 |
| HUMODCA_node_2 | 178 |
| HUMODCA_node_27 | 179 |
| HUMODCA_node_3 | 180 |
| HUMODCA_node_30 | 181 |
| HUMODCA_node_34 | 182 |
| HUMODCA_node_38 | 183 |
| HUMODCA_node_40 | 184 |

TABLE 3
Proteins of interest

| Protein Name | SEQ ID NO: |
|---|---|
| HUMODCA_P9 | 548 |

These sequences are variants of the known protein Ornithine decarboxylase (SwissProt accession identifier DCOR_HUMAN; known also according to the synonyms EC 4.1.1.17; ODC), SEQ ID NO: 620, referred to herein as the previously known protein.

Protein Ornithine decarboxylase (SEQ ID NO:620) is known or believed to have the following function(s): Polyamine biosynthesis; first (rate-limiting) step. The sequence for protein Ornithine decarboxylase is given at the end of the application, as “Ornithine decarboxylase amino acid sequence”. Known polymorphisms for this sequence are as shown in Table 4.

TABLE 4
Amino acid mutations for Known Protein

| SNP position(s) on amino | |
|---|---|
| acid sequence | Comment |
| 415 | Q -> E |

The following GO Annotation(s) apply to the previously known protein. The following annotation(s) were found: polyamine biosynthesis, which are annotation(s) related to Biological Process; and ornithine decarboxylase; lyase, which are annotation(s) related to Molecular Function.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster HUMODCA can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

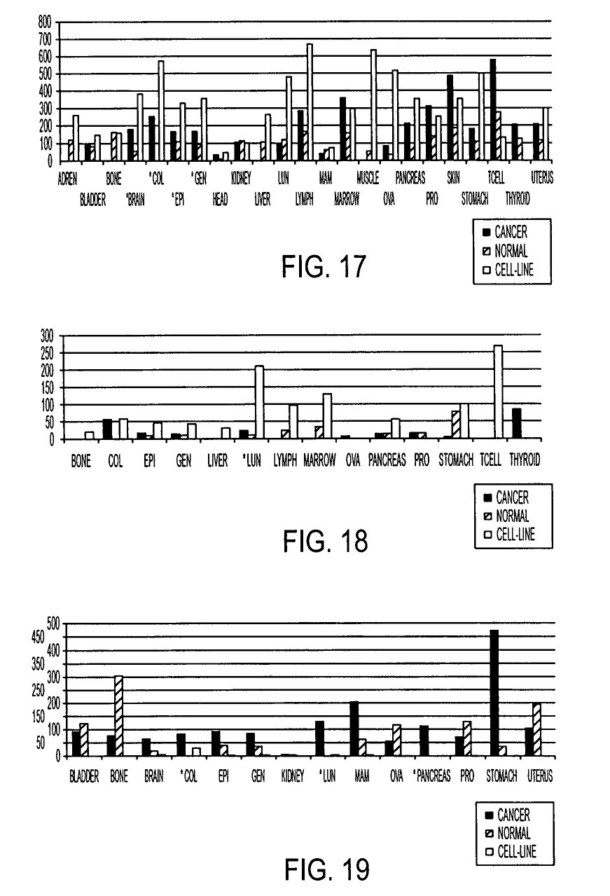

Overall, the following results were obtained as shown with regard to the histograms in FIG. 17 and Table 5. This cluster is overexpressed (at least at a minimum level) in the following pathological conditions: brain malignant tumors, colorectal cancer, epithelial malignant tumors and a mixture of malignant tumors from different tissues.

TABLE 5
Normal tissue distribution

| Name of Tissue | Number |
|---|---|
| Adrenal | 120 |
| Bladder | 82 |
| Bone | 161 |
| Brain | 53 |
| colon | 0 |
| epithelial | 107 |
| general | 94 |
| head and neck | 10 |
| kidney | 114 |
| liver | 107 |
| lung | 120 |
| lymph nodes | 165 |
| breast | 61 |
| bone marrow | 156 |
| muscle | 55 |
| ovary | 36 |
| pancreas | 102 |
| prostate | 140 |
| skin | 188 |
| stomach | 109 |
| T cells | 278 |
| Thyroid | 128 |
| uterus | 118 |

TABLE 6
P values and ratios for expression in cancerous tissue

| Name of Tissue | P1 | P2 | SP1 | R3 | SP2 | R4 |
|---|---|---|---|---|---|---|
| adrenal | 8.3e−01 | 7.8e−01 | 1 | 0.2 | 8.5e−01 | 0.7 |
| bladder | 5.4e−01 | 5.1e−01 | 6.2e−01 | 1.1 | 5.0e−01 | 1.1 |
| bone | 8.3e−01 | 3.2e−01 | 1 | 0.2 | 8.4e−01 | 0.7 |
| brain | 2.6e−01 | 3.8e−02 | 6.5e−04 | 2.8 | 8.7e−10 | 3.6 |
| colon | 2.2e−02 | 5.8e−03 | 1.5e−03 | 6.9 | 6.7e−05 | 9.9 |
| epithelial | 6.4e−02 | 2.7e−03 | 1.4e−03 | 1.5 | 1.6e−12 | 2.1 |
| general | 1.3e−03 | 5.4e−08 | 1.9e−08 | 1.7 | 1.4e−39 | 2.6 |
| head and neck | 1.7e−01 | 1.7e−01 | 1 | 1.2 | 7.5e−01 | 1.3 |
| kidney | 7.7e−01 | 7.6e−01 | 7.1e−01 | 0.8 | 6.6e−01 | 0.9 |
| liver | 7.3e−01 | 5.7e−01 | 1 | 0.3 | 2.4e−01 | 1.2 |
| lung | 7.8e−01 | 5.8e−01 | 7.6e−01 | 0.6 | 7.3e−04 | 1.7 |
| lymph nodes | 3.9e−01 | 2.5e−01 | 1.8e−01 | 1.1 | 1.4e−04 | 2.1 |
| breast | 7.8e−01 | 4.7e−01 | 7.7e−01 | 0.8 | 6.4e−01 | 1.0 |
| bone marrow | 3.4e−01 | 2.6e−01 | 2.8e−01 | 2.1 | 1.6e−01 | 1.2 |
| muscle | 8.5e−01 | 6.1e−01 | 1 | 0.2 | 7.1e−05 | 1.0 |
| ovary | 1.7e−01 | 9.3e−02 | 3.8e−01 | 1.7 | 2.2e−02 | 2.6 |
| pancreas | 2.2e−01 | 3.2e−01 | 5.7e−02 | 1.6 | 6.6e−03 | 1.5 |
| prostate | 5.0e−01 | 4.9e−01 | 3.8e−02 | 1.9 | 4.5e−02 | 1.7 |
| skin | 6.2e−01 | 5.8e−01 | 5.4e−02 | 0.9 | 1.5e−02 | 0.5 |
| stomach | 4.2e−01 | 2.6e−01 | 3.7e−01 | 0.7 | 7.3e−03 | 2.3 |
| T cells | 1 | 1 | 5.5e−01 | 1.5 | 8.1e−01 | 0.9 |
| Thyroid | 8.3e−02 | 8.3e−02 | 5.9e−01 | 1.3 | 5.9e−01 | 1.3 |
| uterus | 4.2e−01 | 2.4e−01 | 1.6e−01 | 1.2 | 4.9e−02 | 1.7 |

As noted above, cluster HUMODCA features 1 transcript(s), which were listed in Table 1 above. These transcript(s) encode for protein(s) which are variant(s) of protein Ornithine decarboxylase (SEQ ID NO:620). A description of each variant protein according to the present invention is now provided.

Variant protein HUMODCA_P9 (SEQ ID NO:548) according to the present invention has an amino acid sequence as given at the end of the application; it is encoded by transcript(s) HUMODCA_T17 (SEQ ID NO:15). An alignment is given to the known protein (Ornithine decarboxylase (SEQ ID NO:620)) at the end of the application. One or more alignments to one or more previously published protein sequences are given at the end of the application. A brief description of the relationship of the variant protein according to the present invention to each such aligned protein is as follows:

Comparison Report Between HUMODCA_P9 (SEQ ID NO:548) and DCOR_HUMAN (SEQ ID NO:620):

1. An isolated chimeric polypeptide encoding for HUMODCA_P9 (SEQ ID NO:548), comprising a first amino acid sequence being at least 70%, optionally at least 80%, preferably at least 85%, more preferably at least 90% and most preferably at least 95% homologous to a polypeptide having the sequence MKSLTATSSMKVLLPRTFWTRKLMKFLLL (SEQ ID NO:1491) corresponding to amino acids 1-29 of HUMODCA_P9 (SEQ ID NO:548), and a second amino acid sequence being at least 90% homologous to LVLRIATDDSKAVCRLSVKFGATLRTSRLLLERAKELNIDVVGVSFHVGSGCTDPETFVQAISDARCVFDM GAEVGFSMYLLDIGGGFPGSEDVKLKFEEITGVINPALDKYFPSDSGVRIIAEPGRYYVASAFTLAVNIIAKK IVLKEQTGSDDEDESSEQTFMYYVNDGVYGSFNCILYDHAHVKPLLQKRPKPDEKYYSSSIWGPTCDGLD RIVERCDLPEMHVGDWMLFENMGAYTVAAASTFNGFQRPTIYYVMSGPAWQLMQQFQNPDFPPEVEEQ DASTLPVSCAWESGMKRHRAACASASINV corresponding to amino acids 151-461 of DCOR_HUMAN (SEQ ID NO:620), which also corresponds to amino acids 30-340 of HUMODCA_P9 (SEQ ID NO:548), wherein said first and second amino acid sequences are contiguous and in a sequential order.

2. An isolated polypeptide encoding for a head of HUMODCA_P9 (SEQ ID NO:548), comprising a polypeptide being at least 70%, optionally at least about 80%, preferably at least about 85%, more preferably at least about 90% and most preferably at least about 95% homologous to the sequence MKSLTATSSMKVLLPRTFWTRKLMKFLLL (SEQ ID NO:1491) of HUMODCA_P9 (SEQ ID NO:548).

Comparison Report Between HUMODCA_P9 (SEQ ID NO:548) and AAA59968 (SEQ ID NO:1387):

1. An isolated chimeric polypeptide encoding for HUMODCA_P9 (SEQ ID NO:548), comprising a first amino acid sequence being at least 70%, optionally at least 80%, preferably at least 85%, more preferably at least 90% and most preferably at least 95% homologous to a polypeptide having the sequence MKSLTATSSMKVLLPRTFWTRKLMKFLLL (SEQ ID NO:1491) corresponding to amino acids 1-29 of HUMODCA_P9 (SEQ ID NO:548), and a second amino acid sequence being at least 90% homologous to LVLRIATDDSKAVCRLSVKFGATLRTSRLLLERAKELNIDVVGVSFHVGSGCTDPETFVQAISDARCVFDM GAEVGFSMYLLDIGGGFPGSEDVKLKFEEITGVINPALDKYFPSDSGVRIIAEPGRYYVASAFTLAVNIIAKK IVLKEQTGSDDEDESSEQTFMYYVNDGVYGSFNCILYDHAHVKPLLQKRPKPDEKYYSSSIWGPTCDGLD RIVERCDLPEMHVGDWMLFENMGAYTVAAASTFNGFQRPTIYYVMSGPAWQLMQQFQNPDFPPEVEEQ DASTLPVSCAWESGMKRHRAACASASINV corresponding to amino acids 40-350 of AAA59968 (SEQ ID NO:1387), which also corresponds to amino acids 30-340 of HUMODCA_P9 (SEQ ID NO:548), wherein said first and second amino acid sequences are contiguous and in a sequential order.

2. An isolated polypeptide encoding for a head of HUMODCA_P9 (SEQ ID NO:548), comprising a polypeptide being at least 70%, optionally at least about 80%, preferably at least about 85%, more preferably at least about 90% and most preferably at least about 95% homologous to the sequence MKSLTATSSMKVLLPRTFWTRKLMKFLLL (SEQ ID NO:1491) of HUMODCA_P9 (SEQ ID NO:548).

Comparison Report Between HUMODCA_P9 (SEQ ID NO:548) and AAH14562 (SEQ ID NO:1388):

1. An isolated chimeric polypeptide encoding for HUMODCA_P9 (SEQ ID NO:548), comprising a first amino acid sequence being at least 70%, optionally at least 80%, preferably at least 85%, more preferably at least 90% and most preferably at least 95% homologous to a polypeptide having the sequence MKSLTATSSMKVLLPRTFWTRKLMKFLLL (SEQ ID NO:1491) corresponding to amino acids 1-29 of HUMODCA_P9 (SEQ ID NO:548), and a second amino acid sequence being at least 90% homologous to LVLRIATDDSKAVCRLSVKFGATLRTSRLLLERAKELNIDVVGVSFHVGSGCTDPETFVQAISDARCVFDM GAEVGFSMYLLDIGGGFPGSEDVKLKFEEITGVINPALDKYFPSDSGVRIIAEPGRYYVASAFTLAVNIIAKK IVLKEQTGSDDEDESSEQTFMYYVNDGVYGSFNCILYDHAHVKPLLQKRPKPDEKYYSSSIWGPTCDGLD RIVERCDLPEMHVGDWMLFENMGAYTVAAASTFNGFQRPTIYYVMSGPAWQLMQQFQNPDFPPEVEEQ DASTLPVSCAWESGMKRHRAACASASINV corresponding to amino acids 86-396 of AAH14562 (SEQ ID NO:1388), which also corresponds to amino acids 30-340 of HUMODCA_P9 (SEQ ID NO:548), wherein said first and second amino acid sequences are contiguous and in a sequential order.

2. An isolated polypeptide encoding for a head of HUMODCA_P9 (SEQ ID NO:548), comprising a polypeptide being at least 70%, optionally at least about 80%, preferably at least about 85%, more preferably at least about 90% and most preferably at least about 95% homologous to the sequence MKSLTATSSMKVLLPRTFWTRKLMKFLLL (SEQ ID NO:1491) of HUMODCA_P9 (SEQ ID NO:548).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMODCA_P9 (SEQ ID NO:548) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein HUMODCA_P9 (SEQ ID NO:548) sequence provides support for the deduced sequence of this variant protein according to the present invention).

TABLE 7
Amino acid mutations

| SNP position(s) on amino | Alternative | Previously |
|---|---|---|
| acid sequence | amino acid(s) | known SNP? |
| 150 | I -> S | No |
| 150 | I -> V | No |
| 262 | F -> L | No |
| 263 | E -> | No |
| 263 | E -> G | No |
| 30 | L -> | No |
| 301 | N -> | No |
| 301 | N -> K | No |
| 309 | E -> K | No |
| 312 | D -> N | No |
| 323 | E -> K | No |
| 329 | H -> P | No |
| 174 | I -> | No |
| 34 | I -> | No |
| 59 | L -> | No |
| 70 | V -> | No |
| 86 | T -> | No |
| 86 | T -> N | No |
| 90 | A -> | No |
| 94 | A -> | No |
| 97 | V -> | No |
| 97 | V -> G | No |
| 198 | N -> D | No |
| 200 | G -> | No |
| 3 | S -> | No |
| 207 | C -> G | No |
| 207 | C -> R | No |
| 223 | P -> | No |
| 262 | F -> | No |

Variant protein HUMODCA_P9 (SEQ ID NO:548) is encoded by the following transcript(s): HUMODCA_T17 (SEQ ID NO:15), for which the sequence(s) is/are given at the end of the application. The coding portion of transcript HUMODCA_T17 (SEQ ID NO:15) is shown in bold; this coding portion starts at position 528 and ends at position 1547. The transcript also has the following SNPs as listed in Table 8 (given according to their position on the nucleotide sequence, with the alternative nucleic acid listed; the last column indicates whether the SNP is known or not; the presence of known SNPs in variant protein HUMODCA_P9 (SEQ ID NO:548) sequence provides support for the deduced sequence of this variant protein according to the present invention).

TABLE 8
Nucleic acid SNPs

| SNP position on | Alternative | Previously |
|---|---|---|
| nucleotide sequence | nucleic acid | known SNP? |
| 28 | C -> G | Yes |
| 210 | C -> | No |
| 536 | T -> | No |
| 615 | T -> | No |
| 628 | T -> | No |
| 703 | T -> | No |
| 736 | T -> | No |
| 784 | C -> | No |
| 784 | C -> A | No |
| 797 | A -> | No |
| 797 | A -> T | No |
| 808 | C -> | No |
| 217 | C -> | No |
| 817 | T -> | No |
| 817 | T -> G | No |
| 869 | C -> T | Yes |
| 975 | A -> G | No |
| 976 | T -> G | No |
| 1048 | T -> | No |
| 1119 | A -> G | No |
| 1127 | C -> | No |
| 1127 | C -> G | No |
| 1146 | T -> C | No |
| 366 | G -> C | No |
| 1146 | T -> G | No |
| 1194 | C -> | No |
| 1283 | T -> C | Yes |
| 1311 | T -> | No |
| 1311 | T -> C | No |
| 1315 | A -> | No |
| 1315 | A -> G | No |
| 1430 | C -> | No |
| 1430 | C -> A | No |
| 1433 | C -> G | No |
| 366 | G -> T | No |
| 1433 | C -> T | Yes |
| 1452 | G -> A | No |
| 1461 | G -> A | No |
| 1494 | G -> A | No |
| 1513 | A -> C | No |
| 1632 | T -> | No |
| 1673 | C -> | No |
| 1739 | T -> | No |
| 1739 | T -> G | No |
| 1742 | T -> C | No |
| 447 | G -> A | Yes |
| 1786 | C -> | No |
| 1786 | C -> G | No |
| 1832 | T -> C | Yes |
| 1877 | C -> T | No |
| 464 | T -> G | Yes |
| 473 | A -> G | Yes |
| 506 | G -> A | Yes |
| 521 | T -> | No |

As noted above, cluster HUMODCA features 17 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HUMODCA_node_{—}1 (SEQ ID NO:168) according to the present invention is supported by 76 libraries. The number of libraries was determined as previously described. This segment can be found in the following transcript(s): HUMODCA_T17 (SEQ ID NO:15). Table 9 below describes the starting and ending position of this segment on each transcript.

TABLE 9
Segment location on transcripts

| | Segment | Segment |
|---|---|---|
| Transcript name | starting position | ending position |
| HUMODCA_T17 (SEQ ID NO: 15) | 118 | 256 |

Segment cluster HUMODCA_node_{—}25 (SEQ ID NO:169) according to the present invention is supported by 190 libraries. The number of libraries was determined as previously described. This segment can be found in the following transcript(s): HUMODCA_T17 (SEQ ID NO:15). Table 10 below describes the starting and ending position of this segment on each transcript.

TABLE 10
Segment location on transcripts

| | Segment | Segment |
|---|---|---|
| Transcript name | starting position | ending position |
| HUMODCA_T17 (SEQ ID NO: 15) | 614 | 748 |

Segment cluster HUMODCA_node_{—}32 (SEQ ID NO:170) according to the present invention is supported by 249 libraries. The number of libraries was determined as previously described. This segment can be found in the following transcript(s): HUMODCA_T17 (SEQ ID NO:15). Table 11 below describes the starting and ending position of this segment on each transcript.

TABLE 11
Segment location on transcripts

| | Segment | Segment |
|---|---|---|
| Transcript name | starting position | ending position |
| HUMODCA_T17 (SEQ ID NO: 15) | 915 | 1077 |

Segment cluster HUMODCA_node_{—}36 (SEQ ID NO:171) according to the present invention is supported by 348 libraries. The number of libraries was determined as previously described. This segment can be found in the following transcript(s): HUMODCA_T17 (SEQ ID NO:15). Table 12 below describes the starting and ending position of this segment on each transcript.

TABLE 12
Segment location on transcripts

| | Segment | Segment |
|---|---|---|
| Transcript name | starting position | ending position |
| HUMODCA_T17 (SEQ ID NO: 15) | 1191 | 1405 |

Segment cluster HUMODCA_node_{—}39 (SEQ ID NO:172) according to the present invention is supported by 297 libraries. The number of libraries was determined as previously described. This segment can be found in the following transcript(s): HUMODCA_T17 (SEQ ID NO:15). Table 13 below describes the starting and ending position of this segment on each transcript.

TABLE 13
Segment location on transcripts

| | Segment | Segment |
|---|---|---|
| Transcript name | starting position | ending position |
| HUMODCA_T17 (SEQ ID NO: 15) | 1461 | 1633 |

Segment cluster HUMODCA_node_{—}41 (SEQ ID NO:173) according to the present invention is supported by 230 libraries. The number of libraries was determined as previously described. This segment can be found in the following transcript(s): HUMODCA_T17 (SEQ ID NO:15). Table 14 below describes the starting and ending position of this segment on each transcript.

TABLE 14
Segment location on transcripts

| | Segment | Segment |
|---|---|---|
| Transcript name | starting position | ending position |
| HUMODCA_T17 (SEQ ID NO: 15) | 1728 | 1893 |

According to an optional embodiment of the present invention, short segments related to the above cluster are also provided. These segments are up to about 120 bp in length, and so are included in a separate description.

Segment cluster HUMODCA_node_{—}0 (SEQ ID NO:174) according to the present invention is supported by 9 libraries. The number of libraries was determined as previously described. This segment can be found in the following transcript(s): HUMODCA_T17 (SEQ ID NO:15). Table 15 below describes the starting and ending position of this segment on each transcript.

TABLE 15
Segment location on transcripts

| | Segment | Segment |
|---|---|---|
| Transcript name | starting position | ending position |
| HUMODCA_T17 (SEQ ID NO: 15) | 1 | 117 |

Segment cluster HUMODCA_node_{—}10 (SEQ ID NO:175) according to the present invention is supported by 107 libraries. The number of libraries was determined as previously described. This segment can be found in the following transcript(s): HUMODCA_T17 (SEQ ID NO:15). Table 16 below describes the starting and ending position of this segment on each transcript.

TABLE 16
Segment location on transcripts

| | Segment | Segment |
|---|---|---|
| Transcript name | starting position | ending position |
| HUMODCA_T17 (SEQ ID NO: 15) | 385 | 494 |

Segment cluster HUMODCA_node_{—}12 (SEQ ID NO:176) according to the present invention is supported by 132 libraries. The number of libraries was determined as previously described. This segment can be found in the following transcript(s): HUMODCA_T17 (SEQ ID NO:15). Table 17 below describes the starting and ending position of this segment on each transcript.

TABLE 17
Segment location on transcripts

| | Segment | Segment |
|---|---|---|
| Transcript name | starting position | ending position |
| HUMODCA_T17 (SEQ ID NO: 15) | 495 | 586 |

Segment cluster HUMODCA_node_{—}13 (SEQ ID NO:177) according to the present invention is supported by 126 libraries. The number of libraries was determined as previously described. This segment can be found in the following transcript(s): HUMODCA_T17 (SEQ ID NO:15). Table 18 below describes the starting and ending position of this segment on each transcript.

TABLE 18
Segment location on transcripts

| | Segment | Segment |
|---|---|---|
| Transcript name | starting position | ending position |
| HUMODCA_T17 (SEQ ID NO: 15) | 587 | 613 |

Segment cluster HUMODCA_node_{—}2 (SEQ ID NO:178) according to the present invention is supported by 81 libraries. The number of libraries was determined as previously described. This segment can be found in the following transcript(s): HUMODCA_T17 (SEQ ID NO:15). Table 19 below describes the starting and ending position of this segment on each transcript.

TABLE 19
Segment location on transcripts

| | Segment | Segment |
|---|---|---|
| Transcript name | starting position | ending position |
| HUMODCA_T17 (SEQ ID NO: 15) | 257 | 328 |

Segment cluster HUMODCA_node_{—}27 (SEQ ID NO:179) according to the present invention is supported by 185 libraries. The number of libraries was determined as previously described. This segment can be found in the following transcript(s): HUMODCA_T17 (SEQ ID NO:15). Table 20 below describes the starting and ending position of this segment on each transcript.

TABLE 20
Segment location on transcripts

| | Segment | Segment |
|---|---|---|
| Transcript name | starting position | ending position |
| HUMODCA_T17 (SEQ ID NO: 15) | 749 | 830 |

Segment cluster HUMODCA_node_{—}3 (SEQ ID NO:180) according to the present invention is supported by 85 libraries. The number of libraries was determined as previously described. This segment can be found in the following transcript(s): HUMODCA_T17 (SEQ ID NO:15). Table 21 below describes the starting and ending position of this segment on each transcript.

TABLE 21
Segment location on transcripts

| | Segment | Segment |
|---|---|---|
| Transcript name | starting position | ending position |
| HUMODCA_T17 (SEQ ID NO: 15) | 329 | 384 |

Segment cluster HUMODCA_node_{—}30 (SEQ ID NO:181) according to the present invention is supported by 196 libraries. The number of libraries was determined as previously described. This segment can be found in the following transcript(s): HUMODCA_T17 (SEQ ID NO:15). Table 22 below describes the starting and ending position of this segment on each transcript.

TABLE 22
Segment location on transcripts

| | Segment | Segment |
|---|---|---|
| Transcript name | starting position | ending position |
| HUMODCA_T17 (SEQ ID NO: 15) | 831 | 914 |

Segment cluster HUMODCA_node_{—}34 (SEQ ID NO:182) according to the present invention is supported by 259 libraries. The number of libraries was determined as previously described. This segment can be found in the following transcript(s): HUMODCA_T17 (SEQ ID NO:15). Table 23 below describes the starting and ending position of this segment on each transcript.

TABLE 23
Segment location on transcripts

| | Segment | Segment |
|---|---|---|
| Transcript name | starting position | ending position |
| HUMODCA_T17 (SEQ ID NO: 15) | 1078 | 1190 |

Segment cluster HUMODCA_node_{—}38 (SEQ ID NO:183) according to the present invention is supported by 272 libraries. The number of libraries was determined as previously described. This segment can be found in the following transcript(s): HUMODCA_T17 (SEQ ID NO:15). Table 24 below describes the starting and ending position of this segment on each transcript.

TABLE 24
Segment location on transcripts

| | Segment | Segment |
|---|---|---|
| Transcript name | starting position | ending position |
| HUMODCA_T17 (SEQ ID NO: 15) | 1406 | 1460 |

Segment cluster HUMODCA_node_{—}40 (SEQ ID NO:184) according to the present invention is supported by 239 libraries. The number of libraries was determined as previously described. This segment can be found in the following transcript(s): HUMODCA_T17 (SEQ ID NO:15). Table 25 below describes the starting and ending position of this segment on each transcript.

TABLE 25
Segment location on transcripts

| | Segment | Segment |
|---|---|---|
| Transcript name | starting position | ending position |
| HUMODCA_T17 (SEQ ID NO: 15) | 1634 | 1727 |

### Variant Protein Alignment to the Previously Known Protein:

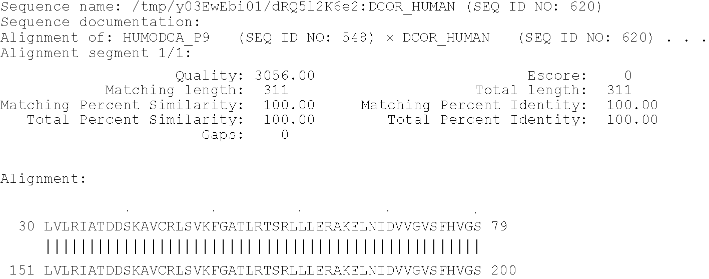

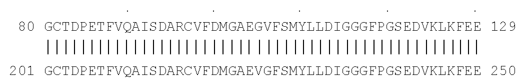

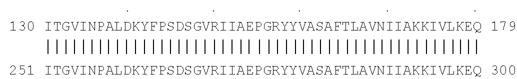

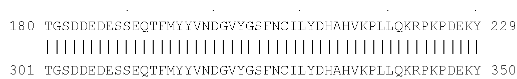

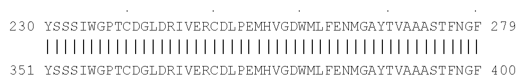

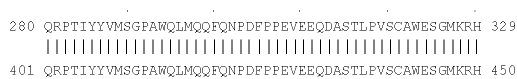

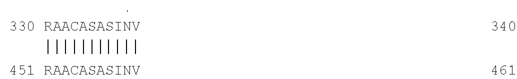

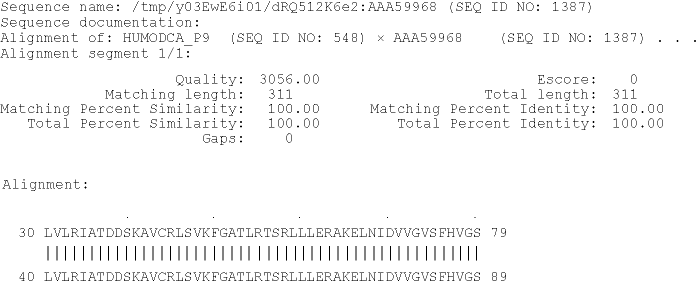

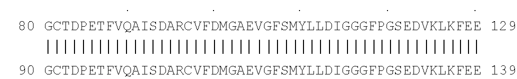

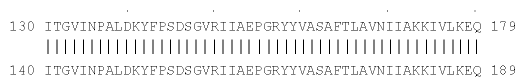

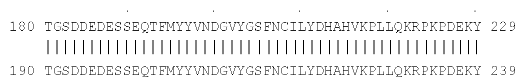

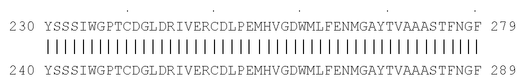

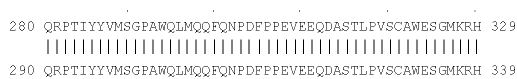

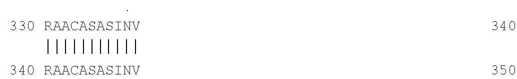

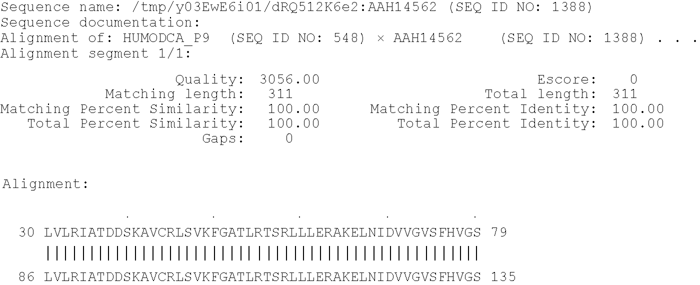

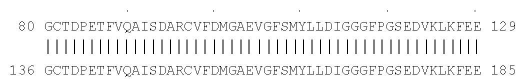

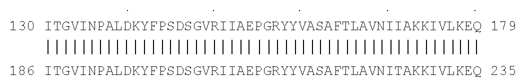

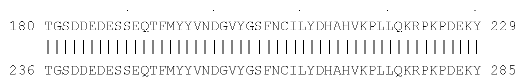

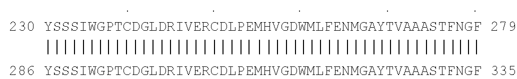

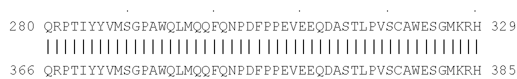

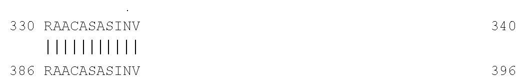

### Description for Cluster R00299

Cluster R00299 features 1 transcript(s) and 12 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| R00299_T2 | 16 |

TABLE 2
Segments of interest

| Segment Name | SEQ ID NO: |
|---|---|
| R00299_node_2 | 185 |
| R00299_node_30 | 186 |
| R00299_node_10 | 187 |
| R00299_node_14 | 188 |
| R00299_node_15 | 189 |
| R00299_node_20 | 190 |
| R00299_node_23 | 191 |
| R00299_node_25 | 192 |
| R00299_node_28 | 193 |
| R00299_node_31 | 194 |
| R00299_node_5 | 195 |
| R00299_node_9 | 196 |

TABLE 3
Proteins of interest

| Protein Name | SEQ ID NO: |
|---|---|
| R00299_P3 | 549 |

These sequences are variants of the known protein Tescalcin (SwissProt accession identifier TESC_HUMAN; known also according to the synonyms TSC), SEQ ID NO: 621, referred to herein as the previously known protein.

Protein Tescalcin is known or believed to have the following function: Binds calcium. The sequence for protein Tescalcin (SEQ ID NO:621) is given at the end of the application, as “Tescalcin amino acid sequence”.

The following GO Annotation(s) apply to the previously known protein. The following annotation(s) were found: calcium binding, which are annotation(s) related to Molecular Function.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster R00299 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 18 and Table 4. This cluster is overexpressed (at least at a minimum level) in the following pathological conditions: lung malignant tumors.

TABLE 4
Normal tissue distribution

| Name of Tissue | Number |
|---|---|
| bone | 0 |
| Colon | 0 |
| epithelial | 11 |
| general | 11 |
| Liver | 0 |
| lung | 10 |
| lymph nodes | 22 |
| bone marrow | 31 |
| ovary | 0 |
| pancreas | 14 |
| prostate | 16 |
| stomach | 76 |
| T cells | 0 |
| Thyroid | 0 |

TABLE 5
P values and ratios for expression in cancerous tissue

| Name of Tissue | P1 | P2 | SP1 | R3 | SP2 | R4 |
|---|---|---|---|---|---|---|
| bone | 1 | 6.7e−01 | 1 | 1.0 | 7.0e−01 | 1.4 |
| colon | 5.0e−02 | 5.3e−02 | 2.4e−01 | 2.8 | 2.1e−01 | 2.8 |
| epithelial | 7.7e−02 | 9.5e−02 | 4.0e−01 | 1.3 | 6.1e−03 | 1.9 |
| general | 2.3e−01 | 2.6e−01 | 5.3e−01 | 1.0 | 2.6e−04 | 1.9 |
| liver | 1 | 4.5e−01 | 1 | 1.0 | 6.9e−01 | 1.5 |
| lung | 4.9e−01 | 2.7e−01 | 6.5e−01 | 1.7 | 5.6e−04 | 3.8 |
| lymph nodes | 8.5e−01 | 8.7e−01 | 1 | 0.5 | 2.0e−01 | 1.1 |
| bone marrow | 8.6e−01 | 8.5e−01 | 1 | 0.5 | 2.3e−01 | 1.4 |
| ovary | 4.0e−01 | 4.4e−01 | 1 | 1.1 | 1 | 1.1 |
| pancreas | 7.2e−01 | 6.9e−01 | 6.7e−01 | 1.0 | 3.5e−01 | 1.5 |
| prostate | 8.7e−01 | 9.1e−01 | 6.7e−01 | 1.0 | 7.5e−01 | 0.9 |
| stomach | 6.6e−01 | 7.5e−01 | 1 | 0.4 | 6.7e−01 | 0.7 |
| T cells | 1 | 6.7e−01 | 1 | 1.0 | 5.2e−01 | 1.8 |
| Thyroid | 1.8e−01 | 1.8e−01 | 6.7e−01 | 1.6 | 6.7e−01 | 1.6 |

As noted above, cluster R00299 features 1 transcript(s), which were listed in Table 1 above. These transcript(s) encode for protein(s) which are variant(s) of protein Tescalcin (SEQ ID NO:621). A description of each variant protein according to the present invention is now provided.

Variant protein R00299_P3 (SEQ ID NO:549) according to the present invention has an amino acid sequence as given at the end of the application; it is encoded by transcript(s) R00299_T2 (SEQ ID NO:16). An alignment is given to the known protein (Tescalcin (SEQ ID NO:621)) at the end of the application. One or more alignments to one or more previously published protein sequences are given at the end of the application. A brief description of the relationship of the variant protein according to the present invention to each such aligned protein is as follows:

Comparison Report Between R00299_P3 (SEQ ID NO:549) and Q9NWT9 (SEQ ID NO:1389):

1. An isolated chimeric polypeptide encoding for R00299_P3 (SEQ ID NO:549), comprising a first amino acid sequence being at least 70%, optionally at least 80%, preferably at least 85%, more preferably at least 90% and most preferably at least 95% homologous to a polypeptide having the sequence MAEKALLCPSSAGLGTWPWVLNSAWPVLPLAVDQGVDWRPRGPV (SEQ ID NO:1492) corresponding to amino acids 1-44 of R00299_P3 (SEQ ID NO:549), second amino acid sequence being at least 90% homologous to SSDQIEQLHRRFKQLSGDQPTIRKENFNNVPDLELNPIRSKIVRAFFDNRNLRKGPSGLADEINFEDFLTIMS YFRPIDTTMDEEQVELSRKEKLRFLFHMYDSDSDGRITLEEYRNV corresponding to amino acids 74-191 of Q9NWT9 (SEQ ID NO:1389), which also corresponds to amino acids 45-162 of R00299_P3 (SEQ ID NO:549), and a third amino acid sequence being at least 70%, optionally at least 80%, preferably at least 85%, more preferably at least 90% and most preferably at least 95% homologous to a polypeptide having the sequence VEELLSGNPHIEKESARSIADGAMMEAASVCMGQMEPDQVYEGITFEDFLKIWQGIDIETKMHVRFLNME TMALCH (SEQ ID NO:1493) corresponding to amino acids 163-238 of R00299_P3 (SEQ ID NO:549), wherein said first, second and third amino acid sequences are contiguous and in a sequential order.

2. An isolated polypeptide encoding for a head of R00299_P3 (SEQ ID NO:549), comprising a polypeptide being at least 70%, optionally at least about 80%, preferably at least about 85%, more preferably at least about 90% and most preferably at least about 95% homologous to the sequence MAEKALLCPSSAGLGTWPWVLNSAWPVLPLAVDQGVDWRPRGPV (SEQ ID NO:1492) of R00299_P3 (SEQ ID NO:549).

3. An isolated polypeptide encoding for a tail of R00299_P3 (SEQ ID NO:549), comprising a polypeptide being at least 70%, optionally at least about 80%, preferably at least about 85%, more preferably at least about 90% and most preferably at least about 95% homologous to the sequence VEELLSGNPHIEKESARSIADGAMMEAASVCMGQMEPDQVYEGITFEDFLKIWQGIDIETKMHVRFLNME TMALCH (SEQ ID NO:1493) in R00299_P3 (SEQ ID NO:549).

Comparison Report Between R00299_P3 (SEQ ID NO:549) and TESC_HUMAN (SEQ ID NO:621):

1. An isolated chimeric polypeptide encoding for R00299_P3 (SEQ ID NO:549), comprising a first amino acid sequence being at least 70%, optionally at least 80%, preferably at least 85%, more preferably at least 90% and most preferably at least 95% homologous to a polypeptide having the sequence MAEKALLCPSSAGLGTWPWVLNSAWPVLPLAVDQGVDWRPRGPV (SEQ ID NO:1492) corresponding to amino acids 1-44 of R00299_P3 (SEQ ID NO:549), and a second amino acid sequence being at least 90% homologous to SSDQIEQLHRRFKQLSGDQPTIRKENFNNVPDLELNPIRSKIVRAFFDNRNLRKGPSGLADEINFEDFLTIMS YFRPIDTTMDEEQVELSRKEKLRFLFHMYDSDSDGRITLEEYRNVVEELLSGNPHIEKESARSIADGAMME AASVCMGQMEPDQVYEGITFEDFLKIWQGIDIETKMHVRFLNMETMALCH corresponding to amino acids 21-214 of TESC_HUMAN (SEQ ID NO:621), which also corresponds to amino acids 45-238 of R00299_P3 (SEQ ID NO:549), wherein said first and second amino acid sequences are contiguous and in a sequential order.

2. An isolated polypeptide encoding for a head of R00299_P3 (SEQ ID NO:549), comprising a polypeptide being at least 70%, optionally at least about 80%, preferably at least about 85%, more preferably at least about 90% and most preferably at least about 95% homologous to the sequence MAEKALLCPSSAGLGTWPWVLNSAWPVLPLAVDQGVDWRPRGPV (SEQ ID NO:1492) of R00299_P3 (SEQ ID NO:549).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein R00299_P3 (SEQ ID NO:549) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein R00299_P3 (SEQ ID NO:549) sequence provides support for the deduced sequence of this variant protein according to the present invention).

TABLE 6
Amino acid mutations

| SNP position(s) on amino acid | | Previously |
|---|---|---|
| sequence | Alternative amino acid(s) | known SNP? |
| 120 | R -> G | No |
| 120 | R -> W | No |

Variant protein R00299_P3 (SEQ ID NO:549) is encoded by the following transcript(s): R00299_T2 (SEQ ID NO:16), for which the sequence(s) is/are given at the end of the application. The coding portion of transcript

R00299_T2 (SEQ ID NO:16) is shown in bold; this coding portion starts at position 142 and ends at position 855. The transcript also has the following SNPs as listed in Table 7 (given according to their position on the nucleotide sequence, with the alternative nucleic acid listed; the last column indicates whether the SNP is known or not; the presence of known SNPs in variant protein R00299_P3 (SEQ ID NO:549) sequence provides support for the deduced sequence of this variant protein according to the present invention).

TABLE 7
Nucleic acid SNPs

| SNP position on | | |
|---|---|---|
| nucleotide sequence | Alternative nucleic acid | Previously known SNP? |
| 177 | C -> A | Yes |
| 499 | C -> G | No |
| 499 | C -> T | No |
| 900 | G -> T | Yes |
| 916 | G -> | No |
| 969 | G -> | No |
| 969 | G -> A | No |
| 987 | A -> C | No |

As noted above, cluster R00299 features 12 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster R00299_node_{—}2 (SEQ ID NO:185) according to the present invention is supported by 3 libraries. The number of libraries was determined as previously described. This segment can be found in the following transcript(s): R00299_T2 (SEQ ID NO:16). Table 8 below describes the starting and ending position of this segment on each transcript.

TABLE 8
Segment location on transcripts

| | Segment | Segment |
|---|---|---|
| Transcript name | starting position | ending position |
| R00299_T2 (SEQ ID NO: 16) | 1 | 271 |

Segment cluster R00299_node_{—}30 (SEQ ID NO:186) according to the present invention is supported by 75 libraries. The number of libraries was determined as previously described. This segment can be found in the following transcript(s): R00299_T2 (SEQ ID NO:16). Table 9 below describes the starting and ending position of this segment on each transcript.

TABLE 9
Segment location on transcripts

| | Segment | Segment |
|---|---|---|
| Transcript name | starting position | ending position |
| R00299_T2 (SEQ ID NO: 16) | 790 | 961 |

According to an optional embodiment of the present invention, short segments related to the above cluster are also provided. These segments are up to about 120 bp in length, and so are included in a separate description.

Segment cluster R00299_node_{—}10 (SEQ ID NO:187) according to the present invention is supported by 46 libraries. The number of libraries was determined as previously described. This segment can be found in the following transcript(s): R00299_T2 (SEQ ID NO:16). Table 10 below describes the starting and ending position of this segment on each transcript.

TABLE 10
Segment location on transcripts

| | Segment | Segment |
|---|---|---|
| Transcript name | starting position | ending position |
| R00299_T2 (SEQ ID NO: 16) | 346 | 422 |

Segment cluster R00299_node_{—}14 (SEQ ID NO:188) according to the present invention is supported by 61 libraries. The number of libraries was determined as previously described. This segment can be found in the following transcript(s): R00299_T2 (SEQ ID NO:16). Table 11 below describes the starting and ending position of this segment on each transcript.

TABLE 11
Segment location on transcripts

| | Segment | Segment |
|---|---|---|
| Transcript name | starting position | ending position |
| R00299_T2 (SEQ ID NO: 16) | 423 | 537 |

Segment cluster R00299_node_{—}15 (SEQ ID NO:189) according to the present invention can be found in the following transcript(s): R00299_T2 (SEQ ID NO:16). Table 12 below describes the starting and ending position of this segment on each transcript.

TABLE 12
Segment location on transcripts

| | Segment | Segment |
|---|---|---|
| Transcript name | starting position | ending position |
| R00299_T2 (SEQ ID NO: 16) | 538 | 562 |

Segment cluster R00299_node_{—}20 (SEQ ID NO:190) according to the present invention is supported by 66 libraries. The number of libraries was determined as previously described. This segment can be found in the following transcript(s): R00299_T2 (SEQ ID NO:16). Table 13 below describes the starting and ending position of this segment on each transcript.

TABLE 13
Segment location on transcripts

| | Segment | Segment |
|---|---|---|
| Transcript name | starting position | ending position |
| R00299_T2 (SEQ ID NO: 16) | 563 | 624 |

Segment cluster R00299_node_{—}23 (SEQ ID NO:191) according to the present invention is supported by 71 libraries. The number of libraries was determined as previously described. This segment can be found in the following transcript(s): R00299_T2 (SEQ ID NO:16). Table 14 below describes the starting and ending position of this segment on each transcript.

TABLE 14
Segment location on transcripts

| | Segment | Segment |
|---|---|---|
| Transcript name | starting position | ending position |
| R00299_T2 (SEQ ID NO: 16) | 625 | 732 |

Segment cluster R00299_node_{—}25 (SEQ ID NO:192) according to the present invention is supported by 62 libraries. The number of libraries was determined as previously described. This segment can be found in the following transcript(s): R00299_T2 (SEQ ID NO:16). Table 15 below describes the starting and ending position of this segment on each transcript.

TABLE 15
Segment location on transcripts

| | Segment | Segment |
|---|---|---|
| Transcript name | starting position | ending position |
| R00299_T2 (SEQ ID NO: 16) | 733 | 780 |

Segment cluster R00299_node_{—}28 (SEQ ID NO:193) according to the present invention can be found in the following transcript(s): R00299_T2 (SEQ ID NO:16). Table 16 below describes the starting and ending position of this segment on each transcript.

TABLE 16
Segment location on transcripts

| | Segment | Segment |
|---|---|---|
| Transcript name | starting position | ending position |
| R00299_T2 (SEQ ID NO: 16) | 781 | 789 |

Segment cluster R00299_node_{—}31 (SEQ ID NO:194) according to the present invention is supported by 48 libraries. The number of libraries was determined as previously described. This segment can be found in the following transcript(s): R00299_T2 (SEQ ID NO:16). Table 17 below describes the starting and ending position of this segment on each transcript.

TABLE 17
Segment location on transcripts

| | Segment | Segment |
|---|---|---|
| Transcript name | starting position | ending position |
| R00299_T2 (SEQ ID NO: 16) | 962 | 1069 |

Segment cluster R00299_node_{—}5 (SEQ ID NO:195) according to the present invention is supported by 45 libraries. The number of libraries was determined as previously described. This segment can be found in the following transcript(s): R00299_T2 (SEQ ID NO:16). Table 18 below describes the starting and ending position of this segment on each transcript.

TABLE 18
Segment location on transcripts

| | Segment | Segment |
|---|---|---|
| Transcript name | starting position | ending position |
| R00299_T2 (SEQ ID NO: 16) | 272 | 341 |

Segment cluster R00299_node_{—}9 (SEQ ID NO:196) according to the present invention can be found in the following transcript(s): R00299_T2 (SEQ ID NO:16). Table 19 below describes the starting and ending position of this segment on each transcript.

TABLE 19
Segment location on transcripts

| | Segment | Segment |
|---|---|---|
| Transcript name | starting position | ending position |
| R00299_T2 (SEQ ID NO: 16) | 342 | 345 |

Microarray (chip) data is also available for this gene as follows. As described above with regard to the cluster itself, various oligonucleotides were tested for being differentially expressed in various disease conditions, particularly cancer. The following oligonucleotide was found to hit this segment with regard to colon cancer, shown in Table 20.

TABLE 20
Oligonucleotide related to this gene

| | Overexpressed | |
|---|---|---|
| Oligonucleotide name | in cancers | Chip reference |
| R00299_0_8_0 (SEQ ID NO: 1404) | Colon cancer | Colon |

### Variant Protein Alignment to the Previously Known Protein:

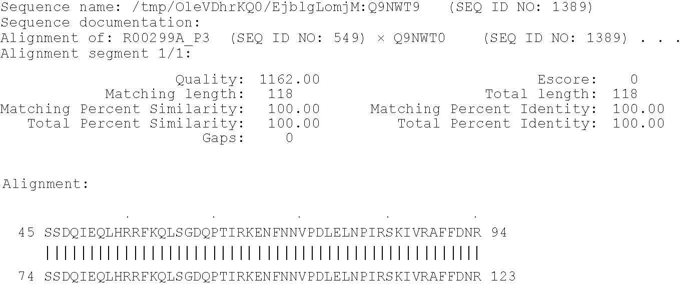

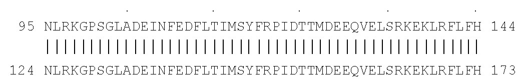

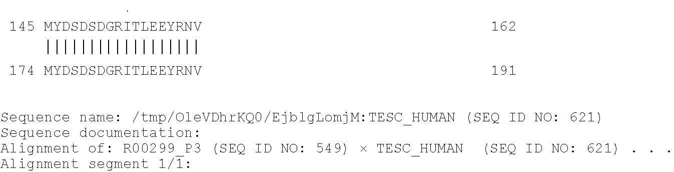

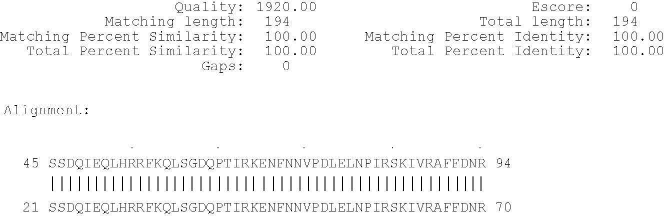

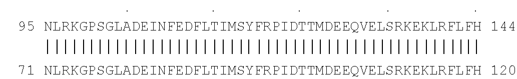

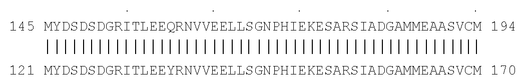

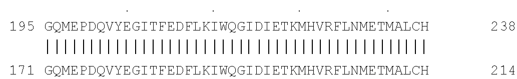

### Description for Cluster Z19178

Cluster Z19178 features 2 transcript(s) and 15 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| Z19178_PEA_1_T5 | 17 |
| Z19178_PEA_1_T9 | 18 |

TABLE 2
Segments of interest

| Segment Name | SEQ ID NO: |
|---|---|
| Z19178_PEA_1_node_15 | 197 |
| Z19178_PEA_1_node_17 | 198 |
| Z19178_PEA_1_node_2 | 199 |
| Z19178_PEA_1_node_22 | 200 |
| Z19178_PEA_1_node_23 | 201 |
| Z19178_PEA_1_node_24 | 202 |
| Z19178_PEA_1_node_10 | 203 |
| Z19178_PEA_1_node_11 | 204 |
| Z19178_PEA_1_node_14 | 205 |
| Z19178_PEA_1_node_18 | 206 |
| Z19178_PEA_1_node_19 | 207 |
| Z19178_PEA_1_node_3 | 208 |
| Z19178_PEA_1_node_4 | 209 |
| Z19178_PEA_1_node_5 | 210 |
| Z19178_PEA_1_node_9 | 211 |

TABLE 3
Proteins of interest

| Protein Name | SEQ ID NO: |
|---|---|
| Z19178_PEA_1_P5 | 550 |
| Z19178_PEA_1_P6 | 551 |

These sequences are variants of the known protein Skeletal muscle LIM-protein 2 (SwissProt accession identifier SLI2_HUMAN; known also according to the synonyms SLIM 2; Four and a half LIM domains protein 3; FHL-3), SEQ ID NO: 622, referred to herein as the previously known protein.

The sequence for protein Skeletal muscle LIM-protein 2 is given at the end of the application, as “Skeletal muscle LIM-protein 2 amino acid sequence” (SEQ ID NO: 622).

The following GO Annotation(s) apply to the previously known protein. The following annotation(s) were found: muscle development, which are annotation(s) related to Biological Process.

The GO assignment relies on information from one or more of the SwissProt/TremB1 Protein knowledgebase, available from <http://www.expasy.chlsprot/>; or Locuslink, available from <http://www.ncbi.nlm.nih.gov/projects/LocusLink/>.

As noted above, cluster Z19178 features 2 transcript(s), which were listed in Table 1 above. These transcript(s) encode for protein(s) which are variant(s) of protein Skeletal muscle LIM-protein 2 (SEQ ID NO:622). A description of each variant protein according to the present invention is now provided.

Variant protein Z19178_PEA_{—}1_P5 (SEQ ID NO:550) according to the present invention has an amino acid sequence as given at the end of the application; it is encoded by transcript(s) Z19178_PEA_{—}1_T5 (SEQ ID NO:17). An alignment is given to the known protein (Skeletal muscle LIM-protein 2 (SEQ ID NO:622)) at the end of the application. One or more alignments to one or more previously published protein sequences are given at the end of the application. A brief description of the relationship of the variant protein according to the present invention to each such aligned protein is as follows:

### Comparison Report Between Z19178_PEA_{—}1_P5 (SEQ ID NO:550) and Q96C98 (SEQ ID NO:1390):

1. An isolated chimeric polypeptide encoding for Z19178_PEA_{—}1_P5 (SEQ ID NO:550), comprising a first amino acid sequence being at least 70%, optionally at least 80%, preferably at least 85%, more preferably at least 90% and most preferably at least 95% homologous to a polypeptide having the sequence GGGGRADWRPKGRWGRGLAPAAGWGAGVRGPGGAGPRSLPRGGVGAALPLAHTVRLQSAASPAARSA PAWPGPQELFYEDRHFHEGCFRCCRCQRSLADEPFTCQDSELLCNDCYCSAFSSQCSACGETV (SEQ ID NO:1494) corresponding to amino acids 1-130 of Z19178_PEA_{—}1_P5 (SEQ ID NO:550), and a second amino acid sequence being at least 90% homologous to MPGSRKLEYGGQTWHEHCFLCSGCEQPLGSRSFVPDKGAHYCVPCYENKFAPRCARCSKTLTQGGVTYR DQPWHRECLVCTGCQTPLAGQQFTSRDEDPYCVACFGELFAPKCSSCKRPIVGLGGGKYVSFEDRHWHH NCFSCARCSTSLVGQGFVPDGDQVLCQGCSQAGP corresponding to amino acids 1-172 of Q96C98 (SEQ ID NO:1390), which also corresponds to amino acids 131-302 of Z19178_PEA_{—}1_P5 (SEQ ID NO:550), wherein said first and second amino acid sequences are contiguous and in a sequential order.

2. An isolated polypeptide encoding for a head of Z19178_PEA_{—}1_P5 (SEQ ID NO:550), comprising a polypeptide being at least 70%, optionally at least about 80%, preferably at least about 85%, more preferably at least about 90% and most preferably at least about 95% homologous to the sequence GGGGRADWRPKGRWGRGLAPAAGWGAGVRGPGGAGPRSLPRGGVGAALPLAHTVRLQSAASPAARSA PAWPGPQELFYEDRHFHEGCFRCCRCQRSLADEPFTCQDSELLCNDCYCSAFSSQCSACGETV (SEQ ID NO:1494) of Z19178_PEA_{—}1_P5 (SEQ ID NO:550).

Comparison Report Between Z19178_PEA_{—}1_P5 (SEQ ID NO:550) and Q9BVA2 (SEQ ID NO:1391):

1. An isolated chimeric polypeptide encoding for Z19178_PEA_L_P5 (SEQ ID NO:550), comprising a first amino acid sequence being at least 70%, optionally at least 80%, preferably at least 85%, more preferably at least 90% and most preferably at least 95% homologous to a polypeptide having the sequence GGGGRADWRPKGRWGRGLAPAAGWGAGVRGPGGAGPRSLPRGGVGAALPLAHTVRLQSAASPAARSA PAWPGPQ corresponding to amino acids 1-74 of Z19178_PEA_{—}1_P5 (SEQ ID NO:550), and a second amino acid sequence being at least 90% homologous to ELFYEDRHFHEGCFRCCRCQRSLADEPFTCQDSELLCNDCYCSAFSSQCSACGETVMPGSRKLEYGGQTW HEHCFLCSGCEQPLGSRSFVPDKGAHYCVPCYENKFAPRCARCSKTLTQGGVTYRDQPWHRECLVCTGC QTPLAGQQFTSRDEDPYCVACFGELFAPKCSSCKRPIVGLGGGKYVSFEDRHWHHNCFSCARCSTSLVGQ GFVPDGDQVLCQGCSQAGP corresponding to amino acids 53-280 of Q9BVA2 (SEQ ID NO:1391), which also corresponds to amino acids 75-302 of Z19178_PEA_{—}1_P5 (SEQ ID NO:550), wherein said first and second amino acid sequences are contiguous and in a sequential order.

2. An isolated polypeptide encoding for a head of Z19178_PEA_{—}1_P5 (SEQ ID NO:550), comprising a polypeptide being at least 70%, optionally at least about 80%, preferably at least about 85%, more preferably at least about 90% and most preferably at least about 95% homologous to the sequence GGGGRADWRPKGRWGRGLAPAAGWGAGVRGPGGAGPRSLPRGGVGAALPLAHTVRLQSAASPAARSA PAWPGPQ of Z19178_PEA_{—}1_P5 (SEQ ID NO:550).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein Z19178_PEA_{—}1_P5 (SEQ ID NO:550) also has the following non-silent SNPs (Single Nucleotide Polymorphisms) as listed in Table 4, (given according to their position(s) on the amino acid sequence, with the alternative amino acid(s) listed; the last column indicates whether the SNP is known or not; the presence of known SNPs in variant protein Z19178_PEA_{—}1_P5 (SEQ ID NO:550) sequence provides support for the deduced sequence of this variant protein according to the present invention).

TABLE 4
Amino acid mutations

| SNP position(s) on amino | Alternative | Previously |
|---|---|---|
| acid sequence | amino acid(s) | known SNP? |
| 247 | K -> | No |
| 278 | T -> N | No |

Variant protein Z19178_PEA_{—}1_P5 (SEQ ID NO:550) is encoded by the following transcript(s): Z19178_PEA_{—}1_T5 (SEQ ID NO:17), for which the sequence(s) is/are given at the end of the application. The coding portion of transcript Z19178_PEA_{—}1_T5 (SEQ ID NO:17) is shown in bold; this coding portion starts at position 1 and ends at position 907. The transcript also has the following SNPs as listed in Table 5 (given according to their position on the nucleotide sequence, with the alternative nucleic acid listed; the last column indicates whether the SNP is known or not; the presence of known SNPs in variant protein Z19178_PEA_{—}1_P5 (SEQ ID NO:550) sequence provides support for the deduced sequence of this variant protein according to the present invention).

TABLE 5
Nucleic acid SNPs

| SNP position on | Alternative | Previously |
|---|---|---|
| nucleotide sequence | nucleic acid | known SNP? |
| 607 | G -> A | Yes |
| 742 | G -> | No |
| 834 | C -> A | No |
| 1082 | C -> G | Yes |
| 1089 | T -> A | Yes |
| 1110 | C -> T | Yes |
| 1236 | C -> T | Yes |
| 1326 | C -> T | No |
| 1450 | C -> T | No |
| 1523 | C -> T | No |

Variant protein Z19178_PEA_{—}1_P6 (SEQ ID NO:551) according to the present invention has an amino acid sequence as given at the end of the application; it is encoded by transcript(s) Z19178_PEA_{—}1_T9 (SEQ ID NO:18). An alignment is given to the known protein (Skeletal muscle LIM-protein 2 (SEQ ID NO:622)) at the end of the application. One or more alignments to one or more previously published protein sequences are given at the end of the application. A brief description of the relationship of the variant protein according to the present invention to each such aligned protein is as follows:

Comparison Report Between Z19178_PEA_{—}1_P6 (SEQ ID NO:551) and Q96C98 (SEQ ID NO:1390):

1. An isolated chimeric polypeptide encoding for Z19178_PEA_{—}1_P6 (SEQ ID NO:551), comprising a first amino acid sequence being at least 70%, optionally at least 80%, preferably at least 85%, more preferably at least 90% and most preferably at least 95% homologous to a polypeptide having the sequence MNPSPARTVSCSAMTATAVRFPRSAPLVGRLSCL (SEQ ID NO:1495) corresponding to amino acids 1-34 of Z19178_PEA_{—}1_P6 (SEQ ID NO:551), and a second amino acid sequence being at least 90% homologous to TLTQGGVTYRDQPWHRECLVCTGCQTPLAGQQFTSRDEDPYCVACFGELFAPKCSSCKRPIVGLGGGKYV SFEDRHWHHNCFSCARCSTSLVGQGFVPDGDQVLCQGCSQAGP corresponding to amino acids 60-172 of Q96C98 (SEQ ID NO:1390), which also corresponds to amino acids 35-147 of Z19178_PEA_{—}1_P6 (SEQ ID NO:551), wherein said first and second amino acid sequences are contiguous and in a sequential order.

2. An isolated polypeptide encoding for a head of Z19178_PEA_{—}1_P6 (SEQ ID NO:551), comprising a polypeptide being at least 70%, optionally at least about 80%, preferably at least about 85%, more preferably at least about 90% and most preferably at least about 95% homologous to the sequence MNPSPARTVSCSAMTATAVRFPRSAPLVGRLSCL (SEQ ID NO:1495) of Z19178_PEA_{—}6 (SEQ ID NO:551).

Comparison Report Between Z19178_PEA_{—}1_P6 (SEQ ID NO:551) and Q9BVA2 (SEQ ID NO:1391):

1. An isolated chimeric polypeptide encoding for Z19178_PEA_{—}1_P6 (SEQ ID NO:551), comprising a first amino acid sequence being at least 70%, optionally at least 80%, preferably at least 85%, more preferably at least 90% and most preferably at least 95% homologous to a polypeptide having the sequence MNPSPARTVSCSAMTATAVRFPRSAPLVGRLSCL (SEQ ID NO:1495) corresponding to amino acids 1-34 of Z19178_PEA_{—}1_P6 (SEQ ID NO:551), and a second amino acid sequence being at least 90% homologous to TLTQGGVTYRDQPWHRECLVCTGCQTPLAGQQFTSRDEDPYCVACFGELFAPKCSSCKRPIVGLGGGKYV SFEDRHWHHNCFSCARCSTSLVGQGFVPDGDQVLCQGCSQAGP corresponding to amino acids 168-280 of Q9BVA2 (SEQ ID NO:1391), which also corresponds to amino acids 35-147 of Z19178_PEA_{—}1_P6 (SEQ ID NO:551), wherein said first and second amino acid sequences are contiguous and in a sequential order.

2. An isolated polypeptide encoding for a head of Z19178_PEA_{—}1_P6 (SEQ ID NO:551), comprising a polypeptide being at least 70%, optionally at least about 80%, preferably at least about 85%, more preferably at least about 90% and most preferably at least about 95% homologous to the sequence MNPSPARTVSCSAMTATAVRFPRSAPLVGRLSCL (SEQ ID NO:1495) of Z19178_PEA_{—}1_P6 (SEQ ID NO:551).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein Z19178_PEA_{—}1_P6 (SEQ ID NO:551) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein Z19178_PEA_{—}1_P6 (SEQ ID NO:551) sequence provides support for the deduced sequence of this variant protein according to the present invention).

TABLE 6
Amino acid mutations

| SNP position(s) on amino | Alternative | Previously |
|---|---|---|
| acid sequence | amino acid(s) | known SNP? |
| 123 | T -> N | No |
| 92 | K -> | No |

Variant protein Z19178_PEA_{—}1_P6 (SEQ ID NO:551) is encoded by the following transcript(s): Z19178_PEA_{—}1_T9 (SEQ ID NO:18), for which the sequence(s) is/are given at the end of the application. The coding portion of transcript Z19178_PEA_{—}1_T9 (SEQ ID NO:18) is shown in bold; this coding portion starts at position 379 and ends at position 819. The transcript also has the following SNPs as listed in Table 7 (given according to their position on the nucleotide sequence, with the alternative nucleic acid listed; the last column indicates whether the SNP is known or not; the presence of known SNPs in variant protein Z19178_PEA_{—}1_P6 (SEQ ID NO:551) sequence provides support for the deduced sequence of this variant protein according to the present invention).

TABLE 7
Nucleic acid SNPs

| SNP position on | Alternative | Previously |
|---|---|---|
| nucleotide sequence | nucleic acid | known SNP? |
| 519 | G -> A | Yes |
| 654 | G -> | No |
| 746 | C -> A | No |
| 994 | C -> G | Yes |
| 1001 | T -> A | Yes |
| 1022 | C -> T | Yes |
| 1148 | C -> T | Yes |
| 1238 | C -> T | No |
| 1362 | C -> T | No |
| 1435 | C -> T | No |

As noted above, cluster Z19178 features 15 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster Z19178_PEA_{—}1_node_{—}15 (SEQ ID NO:197) according to the present invention is supported by 50 libraries. The number of libraries was determined as previously described. This segment can be found in the following transcript(s): Z19178_PEA_{—}1_T5 (SEQ ID NO:17). Table 8 below describes the starting and ending position of this segment on each transcript.

TABLE 8
Segment location on transcripts

| | Segment | |
|---|---|---|
| | starting | Segment |
| Transcript name | position | ending position |
| Z19178_PEA_1_T5 (SEQ ID NO: 17) | 449 | 568 |

Segment cluster Z19178_PEA_{—}1_node_{—}17 (SEQ ID NO:198) according to the present invention is supported by 50 libraries. The number of libraries was determined as previously described. This segment can be found in the following transcript(s): Z19178_PEA_{—}1_T5 (SEQ ID NO:17) and Z19178_PEA_{—}1_T19 (SEQ ID NO:18). Table 9 below describes the starting and ending position of this segment on each transcript.

TABLE 9
Segment location on transcripts

| | Segment | Segment |
|---|---|---|
| | starting | ending |
| Transcript name | position | position |
| Z19178_PEA_1_T5 (SEQ ID NO: 17) | 569 | 699 |
| Z19178_PEA_1_T9 (SEQ ID NO: 18) | 481 | 611 |

Segment cluster Z19178_PEA_{—}1_node_{—}2 (SEQ ID NO:199) according to the present invention is supported by 43 libraries. The number of libraries was determined as previously described. This segment can be found in the following transcript(s): Z19178_PEA_{—}1_T5 (SEQ ID NO:17) and Z19178_PEA_{—}1_T9 (SEQ ID NO:18). Table 10 below describes the starting and ending position of this segment on each transcript.

TABLE 10
Segment location on transcripts

| | Segment | Segment |
|---|---|---|
| | starting | ending |
| Transcript name | position | position |
| Z19178_PEA_1_T5 (SEQ ID NO: 17) | 1 | 217 |
| Z19178_PEA_1_T9 (SEQ ID NO: 18) | 1 | 217 |

Segment cluster Z19178_PEA_{—}1_node_{—}22 (SEQ ID NO:200) according to the present invention is supported by 61 libraries. The number of libraries was determined as previously described. This segment can be found in the following transcript(s): Z19178_PEA_{—}1_T5 (SEQ ID NO:17) and Z19178_PEA_{—}1_T9 (SEQ ID NO:18). Table 11 below describes the starting and ending position of this segment on each transcript.

TABLE 11
Segment location on transcripts

| | Segment | Segment |
|---|---|---|
| | starting | ending |
| Transcript name | position | position |
| Z19178_PEA_1_T5 (SEQ ID NO: 17) | 756 | 1000 |
| Z19178_PEA_1_T9 (SEQ ID NO: 18) | 668 | 912 |

Segment cluster Z19178_PEA_{—}1_node_{—}23 (SEQ ID NO:201) according to the present invention is supported by 81 libraries. The number of libraries was determined as previously described. This segment can be found in the following transcript(s): Z19178_PEA_{—}1_T5 (SEQ ID NO:17) and Z19178_PEA_{—}1_T19 (SEQ ID NO:18). Table 12 below describes the starting and ending position of this segment on each transcript.

TABLE 12
Segment location on transcripts

| | Segment | Segment |
|---|---|---|
| | starting | ending |
| Transcript name | position | position |
| Z19178_PEA_1_T5 (SEQ ID NO: 17) | 1001 | 1404 |
| Z19178_PEA_1_T9 (SEQ ID NO: 18) | 913 | 1316 |

Segment cluster Z19178_PEA_{—}1_node_{—}24 (SEQ ID NO:202) according to the present invention is supported by 58 libraries. The number of libraries was determined as previously described. This segment can be found in the following transcript(s): Z19178_PEA_{—}1_T5 (SEQ ID NO:17) and Z19178_PEA_{—}1_T9 (SEQ ID NO:18). Table 13 below describes the starting and ending position of this segment on each transcript.

TABLE 13
Segment location on transcripts

| | Segment | Segment |
|---|---|---|
| | starting | ending |
| Transcript name | position | position |
| Z19178_PEA_1_T5 (SEQ ID NO: 17) | 1405 | 1554 |
| Z19178_PEA_1_T9 (SEQ ID NO: 18) | 1317 | 1466 |

According to an optional embodiment of the present invention, short segments related to the above cluster are also provided. These segments are up to about 120 bp in length, and so are included in a separate description.

Segment cluster Z19178_PEA_{—}1_node_{—}10 (SEQ ID NO:203) according to the present invention is supported by 60 libraries. The number of libraries was determined as previously described. This segment can be found in the following transcript(s): Z19178_PEA_{—}1_T5 (SEQ ID NO:17) and Z19178_PEA_{—}1_T19 (SEQ ID NO:18). Table 14 below describes the starting and ending position of this segment on each transcript.

TABLE 14
Segment location on transcripts

| | Segment | Segment |
|---|---|---|
| | starting | ending |
| Transcript name | position | position |
| Z19178_PEA_1_T5 (SEQ ID NO: 17) | 277 | 325 |
| Z19178_PEA_1_T9 (SEQ ID NO: 18) | 359 | 407 |

Segment cluster Z19178_PEA_{—}1_node_{—}11 (SEQ ID NO:204) according to the present invention is supported by 56 libraries. The number of libraries was determined as previously described. This segment can be found in the following transcript(s): Z19178_PEA_{—}1_T5 (SEQ ID NO:17) and Z19178_PEA_{—}1_T19 (SEQ ID NO:18). Table 15 below describes the starting and ending position of this segment on each transcript.

TABLE 15
Segment location on transcripts

| | Segment | Segment |
|---|---|---|
| | starting | ending |
| Transcript name | position | position |
| Z19178_PEA_1_T5 (SEQ ID NO: 17) | 326 | 398 |
| Z19178_PEA_1_T9 (SEQ ID NO: 18) | 408 | 480 |

Segment cluster Z19178_PEA_{—}1_node_{—}14 (SEQ ID NO:205) according to the present invention is supported by 53 libraries. The number of libraries was determined as previously described. This segment can be found in the following transcript(s): Z19178_PEA_{—}1_T5 (SEQ ID NO:17). Table 16 below describes the starting and ending position of this segment on each transcript.

TABLE 16
Segment location on transcripts

| | Segment | Segment |
|---|---|---|
| | starting | ending |
| Transcript name | position | position |
| Z19178_PEA_1_T5 (SEQ ID NO: 17) | 399 | 448 |

Segment cluster Z19178_PEA_{—}1_node_{—}18 (SEQ ID NO:206) according to the present invention is supported by 47 libraries. The number of libraries was determined as previously described. This segment can be found in the following transcript(s): Z19178_PEA_{—}1_T5 (SEQ ID NO:17) and Z19178_PEA_{—}1_T9 (SEQ ID NO:18). Table 17 below describes the starting and ending position of this segment on each transcript.

TABLE 17
Segment location on transcripts

| | Segment | Segment |
|---|---|---|
| | starting | ending |
| Transcript name | position | position |
| Z19178_PEA_1_T5 (SEQ ID NO: 17) | 700 | 751 |
| Z19178_PEA_1_T9 (SEQ ID NO: 18) | 612 | 663 |

Segment cluster Z19178_PEA_{—}1_node_{—}19 (SEQ ID NO:207) according to the present invention can be found in the following transcript(s): Z19178_PEA_{—}1_T5 (SEQ ID NO:17) and Z19178_PEA_{—}1_T19 (SEQ ID NO:18). Table 18 below describes the starting and ending position of this segment on each transcript.

TABLE 18
Segment location on transcripts

| | Segment | Segment |
|---|---|---|
| | starting | ending |
| Transcript name | position | position |
| Z19178_PEA_1_T5 (SEQ ID NO: 17) | 752 | 755 |
| Z19178_PEA_1_T9 (SEQ ID NO: 18) | 664 | 667 |

Segment cluster Z19178_PEA_{—}1_node_{—}3 (SEQ ID NO:208) according to the present invention can be found in the following transcript(s): Z19178_PEA_{—}1_T5 (SEQ ID NO:17) and Z19178_PEA_{—}1_T9 (SEQ ID NO:18). Table 19 below describes the starting and ending position of this segment on each transcript.

TABLE 19
Segment location on transcripts

| | Segment | Segment |
|---|---|---|
| | starting | ending |
| Transcript name | position | position |
| Z19178_PEA_1_T5 (SEQ ID NO: 17) | 218 | 223 |
| Z19178_PEA_1_T9 (SEQ ID NO: 18) | 218 | 223 |

Segment cluster Z19178_PEA_{—}1_node_{—}4 (SEQ ID NO:209) according to the present invention is supported by 29 libraries. The number of libraries was determined as previously described. This segment can be found in the following transcript(s): Z19178_PEA_{—}1_T9 (SEQ ID NO:18). Table 20 below describes the starting and ending position of this segment on each transcript.

TABLE 20
Segment location on transcripts

| | Segment | Segment |
|---|---|---|
| | starting | ending |
| Transcript name | position | position |
| Z19178_PEA_1_T9 (SEQ ID NO: 18) | 224 | 266 |

Segment cluster Z19178_PEA_{—}1_node_{—}5 (SEQ ID NO:210) according to the present invention is supported by 31 libraries. The number of libraries was determined as previously described. This segment can be found in the following transcript(s): Z19178_PEA_{—}1_T9 (SEQ ID NO:18). Table 21 below describes the starting and ending position of this segment on each transcript.

TABLE 21
Segment location on transcripts

| | Segment | Segment |
|---|---|---|
| | starting | ending |
| Transcript name | position | position |
| Z19178_PEA_1_T9 (SEQ ID NO: 18) | 267 | 305 |

Segment cluster Z19178_PEA_{—}1_node_{—}9 (SEQ ID NO:211) according to the present invention is supported by 58 libraries. The number of libraries was determined as previously described. This segment can be found in the following transcript(s): Z19178_PEA_{—}1_T5 (SEQ ID NO:17) and Z19178_PEA_{—}1_T9 (SEQ ID NO:18). Table 22 below describes the starting and ending position of this segment on each transcript.

TABLE 22
Segment location on transcripts

| | Segment | Segment |
|---|---|---|
| | starting | ending |
| Transcript name | position | position |
| Z19178_PEA_1_T5 (SEQ ID NO: 17) | 224 | 276 |
| Z19178_PEA_1_T9 (SEQ ID NO: 18) | 306 | 358 |

### Variant Protein Alignment to the Previously Known Protein:

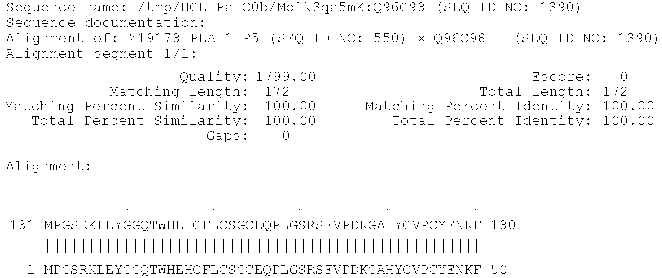

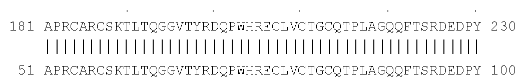

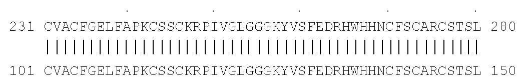

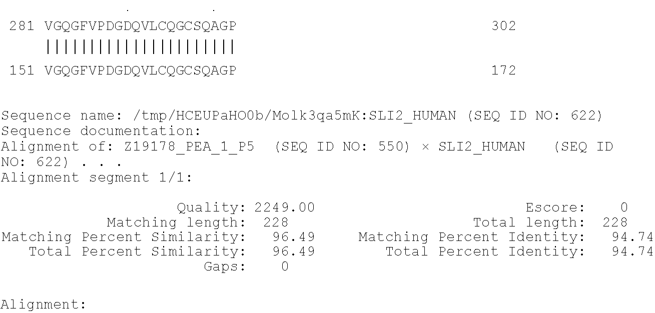

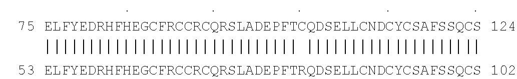

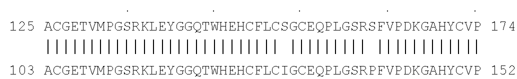

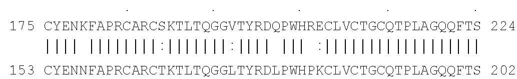

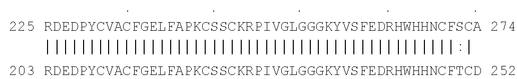

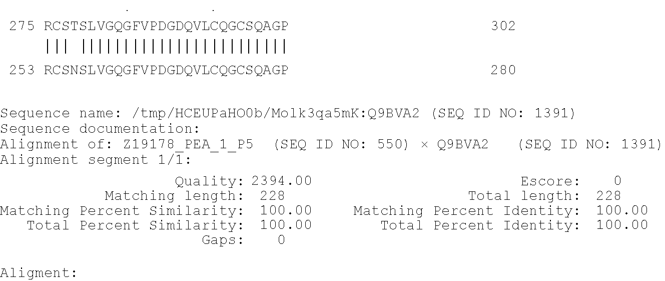

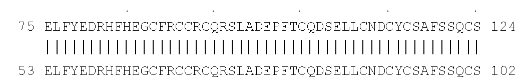

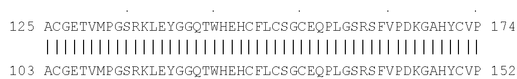

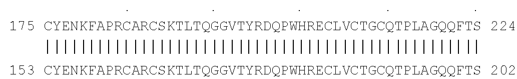

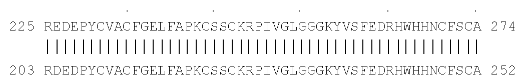

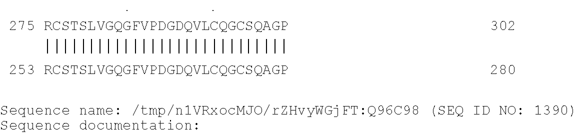

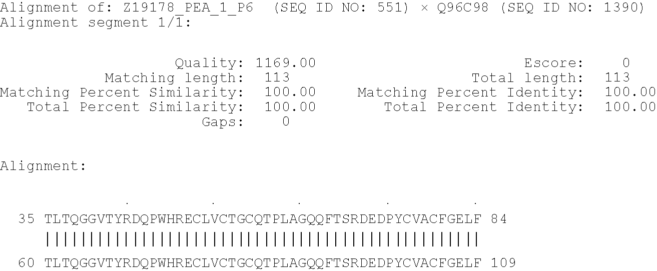

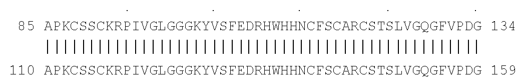

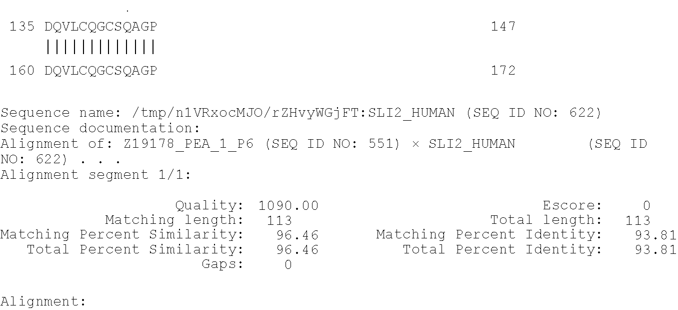

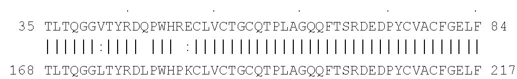

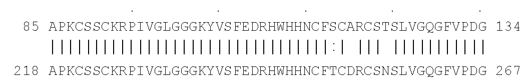

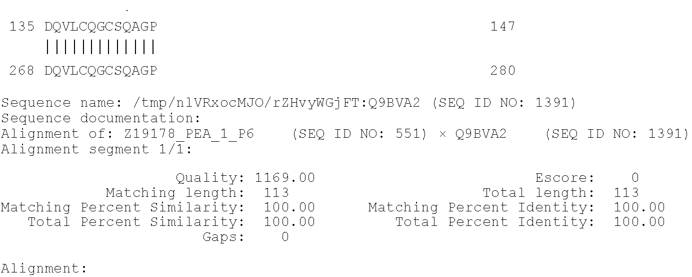

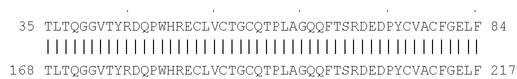

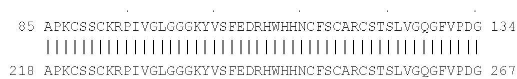

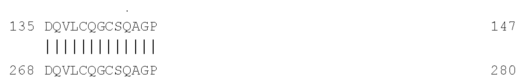

### Description for Cluster S67314

Cluster S67314 features 4 transcript(s) and 8 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| S67314_PEA_1_T4 | 19 |
| S67314_PEA_1_T5 | 20 |
| S67314_PEA_1_T6 | 21 |
| S67314_PEA_1_T7 | 22 |

TABLE 2
Segments of interest

| Segment Name | SEQ ID NO: |
|---|---|
| S67314_PEA_1_node_0 | 212 |
| S67314_PEA_1_node_11 | 213 |
| S67314_PEA_1_node_13 | 214 |
| S67314_PEA_1_node_15 | 215 |
| S67314_PEA_1_node_17 | 216 |
| S67314_PEA_1_node_4 | 217 |
| S67314_PEA_1_node_10 | 218 |
| S67314_PEA_1_node_3 | 219 |

TABLE 3
Proteins of interest

| Protein Name | SEQ ID NO: |
|---|---|
| S67314_PEA_1_P4 | 552 |
| S67314_PEA_1_P5 | 553 |
| S67314_PEA_1_P6 | 554 |
| S67314_PEA_1_P7 | 555 |

These sequences are variants of the known protein Fatty acid-binding protein, heart (SwissProt accession identifier FABH_HUMAN; known also according to the synonyms H-FABP; Muscle fatty acid-binding protein; M-FABP; Mammary-derived growth inhibitor; MDGI), SEQ ID NO: 623, referred to herein as the previously known protein.

Protein Fatty acid-binding protein (SEQ ID NO:623), heart is known or believed to have the following function(s): FABP are thought to play a role in the intracellular transport of long-chain fatty acids and their acyl-CoA esters. The sequence for protein Fatty acid-binding protein, heart is given at the end of the application, as “Fatty acid-binding protein, heart amino acid sequence”. Known polymorphisms for this sequence are as shown in Table 4.

TABLE 4
Amino acid mutations for Known Protein

| SNP position(s) on amino | |
|---|---|
| acid sequence | Comment |
| 1 | V -> A |
| 104 | L -> K |
| 124 | C -> S |
| 129 | E -> Q |

Protein Fatty acid-binding protein (SEQ ID NO:623), heart localization is believed to be Cytoplasmic.

The following GO Annotation(s) apply to the previously known protein. The following annotation(s) were found: negative control of cell proliferation, which are annotation(s) related to Biological Process; and lipid binding, which are annotation(s) related to Molecular Function.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

As noted above, cluster S67314 features 4 transcript(s), which were listed in Table 1 above. These transcript(s) encode for protein(s) which are variant(s) of protein Fatty acid-binding protein (SEQ ID NO:623), heart. A description of each variant protein according to the present invention is now provided.

Variant protein S67314_PEA_{—}1_P4 (SEQ ID NO:552) according to the present invention has an amino acid sequence as given at the end of the application; it is encoded by transcript(s) S67314_PEA_{—}1_T4 (SEQ ID NO:19).

An alignment is given to the known protein (Fatty acid-binding protein (SEQ ID NO:623), heart) at the end of the application. One or more alignments to one or more previously published protein sequences are given at the end of the application. A brief description of the relationship of the variant protein according to the present invention to each such aligned protein is as follows:

Comparison Report Between S67314_PEA_{—}1_P4 (SEQ ID NO:552) and FABH_HUMAN (SEQ ID NO:623):

1. An isolated chimeric polypeptide encoding for S67314_PEA_{—}1_P4 (SEQ ID NO:552), comprising a first amino acid sequence being at least 70%, optionally at least 80%, preferably at least 85%, more preferably at least 90% and most preferably at least 95% homologous to a polypeptide having the sequence MVDAFLGTWKLVDSKNFDDYMKSLGVGFATRQVASMTKPTTIIEKNGDILTLKTHSTFKNTEISFKLGVEF DETTADDRKVKSIVTLDGGKLVHLQKWDGQETTLVRELIDGKLIL corresponding to amino acids 1-116 of FABH_HUMAN (SEQ ID NO:623), which also corresponds to amino acids 1-116 of S67314_PEA_{—}1_P4 (SEQ ID NO:552), and a second amino acid sequence being at least 70%, optionally at least 80%, preferably at least 85%, more preferably at least 90% and most preferably at least 95% homologous to a polypeptide having the sequence VRWATLELYLIGYYYCSFSQACSKKPSPPLRAVEAGTREWLWVRVVSGGNFLCSGFGLTQAGTQILPYRL HDCGQITFSKCNCKTGINNTNLVGLLGSL (SEQ ID NO:1496) corresponding to amino acids 117-215 of S67314_PEA_{—}1_P4 (SEQ ID NO:552), wherein said first and second amino acid sequences are contiguous and in a sequential order.

2. An isolated polypeptide encoding for a tail of S67314_PEA_{—}1_P4 (SEQ ID NO:552), comprising a polypeptide being at least 70%, optionally at least about 80%, preferably at least about 85%, more preferably at least about 90% and most preferably at least about 95% homologous to the sequence VRWATLELYLIGYYYCSFSQACSKKPSPPLRAVEAGTREWLWVRVVSGGNFLCSGFGLTQAGTQILPYRL HDCGQITFSKCNCKTGINNTNLVGLLGSL (SEQ ID NO:1496) in S67314_PEA_{—}1_P4 (SEQ ID NO:552).

Comparison Report Between S67314_PEA_{—}1_P4 (SEQ ID NO:552) and AAP35373 (SEQ ID NO:1392):

1. An isolated chimeric polypeptide encoding for S67314_PEA_{—}1_P4 (SEQ ID NO:552), comprising a first amino acid sequence being at least 90% homologous to MVDAFLGTWKLVDSKNFDDYMKSLGVGFATRQVASMTKPTTIIEKNGDILTLKTHSTFKNTEISFKLGVEF DETTADDRKVKSIVTLDGGKLVHLQKWDGQETTLVRELIDGKLIL corresponding to amino acids 1-116 of AAP35373 (SEQ ID NO:1392), which also corresponds to amino acids 1-116 of S67314_PEA_{—}1_P4 (SEQ ID NO:552), and a second amino acid sequence being at least 70%, optionally at least 80%, preferably at least 85%, more preferably at least 90% and most preferably at least 95% homologous to a polypeptide having the sequence VRWATLELYLIGYYYCSFSQACSKKPSPPLRAVEAGTREWLWVRVVSGGNFLCSGFGLTQAGTQILPYRL HDCGQITFSKCNCKTGINNTNLVGLLGSL (SEQ ID NO:1496) corresponding to amino acids 117-215 of S67314_PEA_{—}1_P4 (SEQ ID NO:552), wherein said first and second amino acid sequences are contiguous and in a sequential order.

2. An isolated polypeptide encoding for a tail of S67314_PEA_{—}1_P4 (SEQ ID NO:552), comprising a polypeptide being at least 70%, optionally at least about 80%, preferably at least about 85%, more preferably at least about 90% and most preferably at least about 95% homologous to the sequence VRWATLELYLIGYYYCSFSQACSKKPSPPLRAVEAGTREWLWVRVVSGGNFLCSGFGLTQAGTQILPYRL HDCGQITFSKCNCKTGINNTNLVGLLGSL (SEQ ID NO:1496) in S67314_PEA_{—}1_P4 (SEQ ID NO:552).

The location of the variant protein was determined according to results from a number of different software programs and analyses, including analyses from SignalP and other specialized programs. The variant protein is believed to be located as follows with regard to the cell: intracellularly. The protein localization is believed to be intracellular because neither of the trans-membrane region prediction programs predicted a trans-membrane region for this protein. In addition both signal-peptide prediction programs predict that this protein is a non-secreted protein.

Variant protein S67314_PEA_{—}1_P4 (SEQ ID NO:552) also has the following non-silent SNPs (Single Nucleotide Polymorphisms) as listed in Table 5, (given according to their position(s) on the amino acid sequence, with the alternative amino acid(s) listed; the last column indicates whether the SNP is known or not; the presence of known SNPs in variant protein S67314_PEA_{—}1_P4 (SEQ ID NO:552) sequence provides support for the deduced sequence of this variant protein according to the present invention).

TABLE 5
Amino acid mutations

| SNP position(s) on | Alternative | Previously |
|---|---|---|
| amino acid sequence | amino acid(s) | known SNP? |
| 53 | K -> R | Yes |

Variant protein S67314_PEA_{—}1_P4 (SEQ ID NO:552) is encoded by the following transcript(s): S67314_PEA_{—}1_T4 (SEQ ID NO:19), for which the sequence(s) is/are given at the end of the application. The coding portion of transcript S67314_PEA_{—}1_T4 (SEQ ID NO:19) is shown in bold; this coding portion starts at position 925 and ends at position 1569. The transcript also has the following SNPs as listed in Table 6 (given according to their position on the nucleotide sequence, with the alternative nucleic acid listed; the last column indicates whether the SNP is known or not; the presence of known SNPs in variant protein S67314_PEA_{—}1_P4 (SEQ ID NO:552) sequence provides support for the deduced sequence of this variant protein according to the present invention).

TABLE 6
Nucleic acid SNPs

| SNP position on | Alternative | Previously |
|---|---|---|
| nucleotide sequence | nucleic acid | known SNP? |
| 580 | T -> C | Yes |
| 1082 | A -> G | Yes |
| 1670 | A -> C | Yes |

Variant protein S67314_PEA_{—}1_P5 (SEQ ID NO:553) according to the present invention has an amino acid sequence as given at the end of the application; it is encoded by transcript(s) S67314_PEA_{—}1_T5 (SEQ ID NO:20). An alignment is given to the known protein (Fatty acid-binding protein (SEQ ID NO:623), heart) at the end of the application. One or more alignments to one or more previously published protein sequences are given at the end of the application. A brief description of the relationship of the variant protein according to the present invention to each such aligned protein is as follows:

Comparison Report Between S67314_PEA_{—}1_P5 (SEQ ID NO:553) and FABH_HUMAN (SEQ ID NO:623):

1. An isolated chimeric polypeptide encoding for S67314_PEA_{—}1_P5 (SEQ ID NO:553), comprising a first amino acid sequence being at least 70%, optionally at least 80%, preferably at least 85%, more preferably at least 90% and most preferably at least 95% homologous to a polypeptide having the sequence MVDAFLGTWKLVDSKNFDDYMKSLGVGFATRQVASMTKPTTIIEKNGDILTLKTHSTFKNTEISFKLGVEF DETTADDRKVKSIVTLDGGKLVHLQKWDGQETTLVRELIDGKLIL corresponding to amino acids 1-116 of FABH_HUMAN (SEQ ID NO:623), which also corresponds to amino acids 1-116 of S67314_PEA_{—}1_P5 (SEQ ID NO:553), and a second amino acid sequence being at least 70%, optionally at least 80%, preferably at least 85%, more preferably at least 90% and most preferably at least 95% homologous to a polypeptide having the sequence DVLTAWPSIYRRQVKVLREDEITILPWHLQWSREKATKLLRPTLPSYNNHGWEELRVGKSIV (SEQ ID NO:1497) corresponding to amino acids 117-178 of S67314_PEA_{—}1_P5 (SEQ ID NO:553), wherein said first and second amino acid sequences are contiguous and in a sequential order.

2. An isolated polypeptide encoding for a tail of S67314_PEA_{—}1_P5 (SEQ ID NO:553), comprising a polypeptide being at least 70%, optionally at least about 80%, preferably at least about 85%, more preferably at least about 90% and most preferably at least about 95% homologous to the sequence DVLTAWPSIYRRQVKVLREDEITILPWHLQWSREKATKLLRPTLPSYNNHGWEELRVGKSIV (SEQ ID NO:1497) in S67314_PEA_{—}1_P5 (SEQ ID NO:553).

Comparison Report Between S67314_PEA_{—}1_P5 (SEQ ID NO:553) and AAP35373 (SEQ ID NO:1392):

1. An isolated chimeric polypeptide encoding for S67314_PEA_{—}1_P5 (SEQ ID NO:553), comprising a first amino acid sequence being at least 90% homologous to MVDAFLGTWKLVDSKNFDDYMKSLGVGFATRQVASMTKPTTIIEKNGDILTLKTHSTFKNTEISFKLGVEF DETTADDRKVKSIVTLDGGKLVHLQKWDGQETTLVRELIDGKLIL corresponding to amino acids 1-116 of AAP35373 (SEQ ID NO:1392), which also corresponds to amino acids 1-116 of S67314_PEA_{—}1_P5 (SEQ ID NO:553), and a second amino acid sequence being at least 70%, optionally at least 80%, preferably at least 85%, more preferably at least 90% and most preferably at least 95% homologous to a polypeptide having the sequence DVLTAWPSIYRRQVKVLREDEITILPWHLQWSREKATKLLRPTLPSYNNHGWEELRVGKSIV (SEQ ID NO:1497) corresponding to amino acids 117-178 of S67314_PEA_{—}1_P5 (SEQ ID NO:553), wherein said first and second amino acid sequences are contiguous and in a sequential order.

2. An isolated polypeptide encoding for a tail of S67314_PEA_{—}1_P5 (SEQ ID NO:553), comprising a polypeptide being at least 70%, optionally at least about 80%, preferably at least about 85%, more preferably at least about 90% and most preferably at least about 95% homologous to the sequence DVLTAWPSIYRRQVKVLREDEITILPWHLQWSREKATKLLRPTLPSYNNHGWEELRVGKSIV (SEQ ID NO:1497) in S67314_PEA_{—}1_P5 (SEQ ID NO:553).

The location of the variant protein was determined according to results from a number of different software programs and analyses, including analyses from SignalP and other specialized programs. The variant protein is believed to be located as follows with regard to the cell: intracellularly. The protein localization is believed to be intracellular because neither of the trans-membrane region prediction programs predicted a trans-membrane region for this protein. In addition both signal-peptide prediction programs predict that this protein is a non-secreted protein.

Variant protein S67314_PEA_{—}1_P5 (SEQ ID NO:553) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein S67314_PEA_{—}1_P5 (SEQ ID NO:553) sequence provides support for the deduced sequence of this variant protein according to the present invention).

TABLE 7
Amino acid mutations

| SNP position(s) on | Alternative | Previously |
|---|---|---|
| amino acid sequence | amino acid(s) | known SNP? |
| 53 | K -> R | Yes |

Variant protein S67314_PEA_{—}1_P5 (SEQ ID NO:553) is encoded by the following transcript(s): S67314_PEA_{—}1_T5 (SEQ ID NO:20), for which the sequence(s) is/are given at the end of the application. The coding portion of transcript S67314_PEA_{—}1_T5 (SEQ ID NO:20) is shown in bold; this coding portion starts at position 925 and ends at position 1458. The transcript also has the following SNPs as listed in Table 8 (given according to their position on the nucleotide sequence, with the alternative nucleic acid listed; the last column indicates whether the SNP is known or not; the presence of known SNPs in variant protein S67314_PEA_{—}1_P5 (SEQ ID NO:553) sequence provides support for the deduced sequence of this variant protein according to the present invention).

TABLE 8
Nucleic acid SNPs

| SNP position on | Alternative | Previously |
|---|---|---|
| nucleotide sequence | nucleic acid | known SNP? |
| 580 | T -> C | Yes |
| 1082 | A -> G | Yes |
| 1326 | A -> G | Yes |

Variant protein S67314_PEA_{—}1_P6 (SEQ ID NO:554) according to the present invention has an amino acid sequence as given at the end of the application; it is encoded by transcript(s) S67314_PEA_{—}1_T6 (SEQ ID NO:21). An alignment is given to the known protein (Fatty acid-binding protein (SEQ ID NO:623), heart) at the end of the application. One or more alignments to one or more previously published protein sequences are given at the end of the application. A brief description of the relationship of the variant protein according to the present invention to each such aligned protein is as follows:

Comparison Report Between S67314_PEA_{—}1_P6 (SEQ ID NO:554) and FABH_HUMAN (SEQ ID NO:623):

1. An isolated chimeric polypeptide encoding for S67314_PEA_{—}1_P6 (SEQ ID NO:554), comprising a first amino acid sequence being at least 70%, optionally at least 80%, preferably at least 85%, more preferably at least 90% and most preferably at least 95% homologous to a polypeptide having the sequence MVDAFLGTWKLVDSKNFDDYMKSLGVGFATRQVASMTKPTTIIEKNGDILTLKTHSTFKNTEISFKLGVEF DETTADDRKVKSIVTLDGGKLVHLQKWDGQETTLVRELIDGKLIL corresponding to amino acids 1-116 of FABH_HUMAN (SEQ ID NO:623), which also corresponds to amino acids 1-116 of S67314_PEA_{—}1_P6 (SEQ ID NO:554), and a second amino acid sequence being at least 70%, optionally at least 80%, preferably at least 85%, more preferably at least 90% and most preferably at least 95% homologous to a polypeptide having the sequence MEKLQLRNVK (SEQ ID NO:1498) corresponding to amino acids 117-126 of S67314_PEA_{—}1_P6 (SEQ ID NO:554), wherein said first and second amino acid sequences are contiguous and in a sequential order.

2. An isolated polypeptide encoding for a tail of S67314_PEA_{—}1_P6 (SEQ ID NO:554), comprising a polypeptide being at least 70%, optionally at least about 80%, preferably at least about 85%, more preferably at least about 90% and most preferably at least about 95% homologous to the sequence MEKLQLRNVK (SEQ ID NO:1498) in S67314_PEA_{—}1_P6 (SEQ ID NO:554).

Comparison Report Between S67314_PEA_{—}1_P6 (SEQ ID NO:554) and AAP35373 (SEQ ID NO:1392):

1. An isolated chimeric polypeptide encoding for S67314_PEA_{—}1_P6 (SEQ ID NO:554), comprising a first amino acid sequence being at least 90% homologous to MVDAFLGTWKLVDSKNFDDYMKSLGVGFATRQVASMTKPTTIIEKNGDILTLKTHSTFKNTEISFKLGVEF DETTADDRKVKSIVTLDGGKLVHLQKWDGQETTLVRELIDGKLIL corresponding to amino acids 1-116 of AAP35373 (SEQ ID NO:1392), which also corresponds to amino acids 1-116 of S67314_PEA_{—}1_P6 (SEQ ID NO:554), and a second amino acid sequence being at least 70%, optionally at least 80%, preferably at least 85%, more preferably at least 90% and most preferably at least 95% homologous to a polypeptide having the sequence MEKLQLRNVK (SEQ ID NO:1498) corresponding to amino acids 117-126 of S67314_PEA_{—}1_P6 (SEQ ID NO:554), wherein said first and second amino acid sequences are contiguous and in a sequential order.

2. An isolated polypeptide encoding for a tail of S67314_PEA_{—}1_P6 (SEQ ID NO:554), comprising a polypeptide being at least 70%, optionally at least about 80%, preferably at least about 85%, more preferably at least about 90% and most preferably at least about 95% homologous to the sequence MEKLQLRNVK (SEQ ID NO:1498) in S67314_PEA_{—}1_P6 (SEQ ID NO:554).

The location of the variant protein was determined according to results from a number of different software programs and analyses, including analyses from SignalP and other specialized programs. The variant protein is believed to be located as follows with regard to the cell: intracellularly. The protein localization is believed to be intracellular because neither of the trans-membrane region prediction programs predicted a trans-membrane region for this protein. In addition both signal-peptide prediction programs predict that this protein is a non-secreted protein.

Variant protein S67314_PEA_{—}1_P6 (SEQ ID NO:554) also has the following non-silent SNPs (Single Nucleotide Polymorphisms) as listed in Table 9, (given according to their position(s) on the amino acid sequence, with the alternative amino acid(s) listed; the last column indicates whether the SNP is known or not; the presence of known SNPs in variant protein S67314_PEA_{—}1_P6 (SEQ ID NO:554) sequence provides support for the deduced sequence of this variant protein according to the present invention).

TABLE 9
Amino acid mutations

| SNP position(s) on | Alternative | Previously |
|---|---|---|
| amino acid sequence | amino acid(s) | known SNP? |
| 53 | K -> R | Yes |

Variant protein S67314_PEA_{—}1_P6 (SEQ ID NO:554) is encoded by the following transcript(s): S67314_PEA_{—}1_T6 (SEQ ID NO:21), for which the sequence(s) is/are given at the end of the application. The coding portion of transcript S67314_PEA_{—}1_T6 (SEQ ID NO:21) is shown in bold; this coding portion starts at position 925 and ends at position 1302. The transcript also has the following SNPs as listed in Table 10 (given according to their position on the nucleotide sequence, with the alternative nucleic acid listed; the last column indicates whether the SNP is known or not; the presence of known SNPs in variant protein S67314_PEA_{—}1_P6 (SEQ ID NO:554) sequence provides support for the deduced sequence of this variant protein according to the present invention).

TABLE 10
Nucleic acid SNPs

| SNP position on | Alternative | Previously |
|---|---|---|
| nucleotide sequence | nucleic acid | known SNP? |
| 580 | T -> C | Yes |
| 1082 | A -> G | Yes |
| 1444 | T -> C | Yes |

Variant protein S67314_PEA_{—}1_P7 (SEQ ID NO:555) according to the present invention has an amino acid sequence as given at the end of the application; it is encoded by transcript(s) S67314_PEA_{—}1_T7 (SEQ ID NO:22). An alignment is given to the known protein (Fatty acid-binding protein (SEQ ID NO:623), heart) at the end of the application. One or more alignments to one or more previously published protein sequences are given at the end of the application. A brief description of the relationship of the variant protein according to the present invention to each such aligned protein is as follows:

Comparison Report Between S67314_PEA_{—}1_P7 (SEQ ID NO:555) and FABH_HUMAN (SEQ ID NO:623):

1. An isolated chimeric polypeptide encoding for S67314_PEA_{—}1_P7 (SEQ ID NO:555), comprising a first amino acid sequence being at least 90% homologous to MVDAFLGTWKLVDSKNFDDYMKSL corresponding to amino acids 1-24 of FABH_HUMAN (SEQ ID NO:623), which also corresponds to amino acids 1-24 of S67314_PEA_{—}1_P7 (SEQ ID NO:555), second amino acid sequence being at least 70%, optionally at least 80%, preferably at least 85%, more preferably at least 90% and most preferably at least 95% homologous to a polypeptide having the sequence AHILITFPLPS (SEQ ID NO:1499) corresponding to amino acids 25-35 of S67314_PEA_{—}1_P7 (SEQ ID NO:555), and a third amino acid sequence being at least 90% homologous to GVGFATRQVASMTKPTTIIEKNGDILTLKTHSTFKNTEISFKLGVEFDETTADDRKVKSIVTLDGGKLVHLQ KWDGQETTLVRELIDGKLILTLTHGTAVCTRTYEKEA corresponding to amino acids 25-133 of FABH_HUMAN (SEQ ID NO:623), which also corresponds to amino acids 36-144 of S67314_PEA_{—}1_P7 (SEQ ID NO:555), wherein said first, second, third and fourth amino acid sequences are contiguous and in a sequential order.

2. An isolated polypeptide encoding for an edge portion of S67314_PEA_{—}1_P7 (SEQ ID NO:555) comprising an amino acid sequence being at least 70%, optionally at least about 80%, preferably at least about 85%, more preferably at least about 90% and most preferably at least about 95% homologous to the sequence encoding for AHILITFPLPS (SEQ ID NO:1499), corresponding to S67314_PEA_{—}1_P7 (SEQ ID NO:555).

Comparison Report Between S67314_PEA_{—}1_P7 (SEQ ID NO:555) and AAP35373 (SEQ ID NO:1392):

1. An isolated chimeric polypeptide encoding for S67314_PEA_{—}1_P7 (SEQ ID NO:555), comprising a first amino acid sequence being at least 90% homologous to MVDAFLGTWKLVDSKNFDDYMKSL corresponding to amino acids 1-24 of AAP35373 (SEQ ID NO:1392), which also corresponds to amino acids 1-24 of S67314_PEA_{—}1_P7 (SEQ ID NO:555), second amino acid sequence being at least 70%, optionally at least 80%, preferably at least 85%, more preferably at least 90% and most preferably at least 95% homologous to a polypeptide having the sequence AHILITFPLPS (SEQ ID NO:1499) corresponding to amino acids 25-35 of S67314_PEA_{—}1_P7 (SEQ ID NO:555), and a third amino acid sequence being at least 90% homologous to GVGFATRQVASMTKPTTHEKNGDILTLKTHSTFKNTEISFKLGVEFDETTADDRKVKSIVTLDGGKLVHLQ KWDGQETTLVRELIDGKLILTLTHGTAVCTRTYEKEA corresponding to amino acids 25-133 of AAP35373 (SEQ ID NO:1392), which also corresponds to amino acids 36-144 of S67314_PEA_{—}1_P7 (SEQ ID NO:555), wherein said first, second and third amino acid sequences are contiguous and in a sequential order.

2. An isolated polypeptide encoding for an edge portion of S67314_PEA_{—}1_P7 (SEQ ID NO:555) comprising an amino acid sequence being at least 70%, optionally at least about 80%, preferably at least about 85%, more preferably at least about 90% and most preferably at least about 95% homologous to the sequence encoding for AHILITFPLPS (SEQ ID NO:1499), corresponding to S67314_PEA_{—}1_P7 (SEQ ID NO:555).

The location of the variant protein was determined according to results from a number of different software programs and analyses, including analyses from SignalP and other specialized programs. The variant protein is believed to be located as follows with regard to the cell: intracellularly. The protein localization is believed to be intracellular because neither of the trans-membrane region prediction programs predicted a trans-membrane region for this protein. In addition both signal-peptide prediction programs predict that this protein is a non-secreted protein.

Variant protein S67314_PEA_{—}1_P7 (SEQ ID NO:555) also has the following non-silent SNPs (Single Nucleotide Polymorphisms) as listed in Table 11, (given according to their position(s) on the amino acid sequence, with the alternative amino acid(s) listed; the last column indicates whether the SNP is known or not; the presence of known SNPs in variant protein S67314_PEA_{—}1_P7 (SEQ ID NO:555) sequence provides support for the deduced sequence of this variant protein according to the present invention).

TABLE 11
Amino acid mutations

| SNP position(s) on | Alternative | Previously |
|---|---|---|
| amino acid sequence | amino acid(s) | known SNP? |
| 64 | K -> R | Yes |

Variant protein S67314_PEA_{—}1_P7 (SEQ ID NO:555) is encoded by the following transcript(s): S67314_PEA_{—}1_T7 (SEQ ID NO:22), for which the sequence(s) is/are given at the end of the application. The coding portion of transcript S67314_PEA_{—}1_T7 (SEQ ID NO:22) is shown in bold; this coding portion starts at position 925 and ends at position 1356. The transcript also has the following SNPs as listed in Table 12 (given according to their position on the nucleotide sequence, with the alternative nucleic acid listed; the last column indicates whether the SNP is known or not; the presence of known SNPs in variant protein S67314_PEA_{—}1_P7 (SEQ ID NO:555) sequence provides support for the deduced sequence of this variant protein according to the present invention).

TABLE 12
Nucleic acid SNPs

| SNP position on | Alternative | Previously |
|---|---|---|
| nucleotide sequence | nucleic acid | known SNP? |
| 580 | T -> C | Yes |
| 1115 | A -> G | Yes |
| 2772 | G -> A | Yes |
| 2896 | C -> A | Yes |
| 2918 | G -> C | Yes |
| 3003 | A -> G | Yes |
| 3074 | T -> G | Yes |
| 1344 | T -> C | Yes |
| 1522 | -> T | No |
| 1540 | -> A | No |
| 1540 | -> T | No |
| 1578 | G -> A | Yes |
| 1652 | G -> A | Yes |
| 2263 | G -> A | Yes |
| 2605 | T -> C | Yes |

As noted above, cluster S67314 features 8 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster S67314_PEA_{—}1_node_{—}0 (SEQ ID NO:212) according to the present invention is supported by 90 libraries. The number of libraries was determined as previously described. This segment can be found in the following transcript(s): S67314_PEA_{—}1_T4 (SEQ ID NO:19), S67314_PEA_{—}1_T5 (SEQ ID NO:20), S67314_PEA_{—}1_T6 (SEQ ID NO:21) and S67314_PEA_{—}1_T7 (SEQ ID NO:22). Table 13 below describes the starting and ending position of this segment on each transcript.

TABLE 13
Segment location on transcripts

| | Segment | Segment |
|---|---|---|
| | starting | ending |
| Transcript name | position | position |
| S67314_PEA_1_T4 (SEQ ID NO: 19) | 1 | 997 |
| S67314_PEA_1_T5 (SEQ ID NO: 20) | 1 | 997 |
| S67314_PEA_1_T6 (SEQ ID NO: 21) | 1 | 997 |
| S67314_PEA_1_T7 (SEQ ID NO: 22) | 1 | 997 |

Segment cluster S67314_PEA_{—}1_node_{—}11 (SEQ ID NO:213) according to the present invention is supported by 6 libraries. The number of libraries was determined as previously described. This segment can be found in the following transcript(s): S67314_PEA_{—}1_T4 (SEQ ID NO:19). Table 14 below describes the starting and ending position of this segment on each transcript.

TABLE 14
Segment location on transcripts

| | Segment | Segment |
|---|---|---|
| | starting | ending |
| Transcript name | position | position |
| S67314_PEA_1_T4 (SEQ ID NO: 19) | 1273 | 2110 |

Segment cluster S67314_PEA_{—}1_node_{—}13 (SEQ ID NO:214) according to the present invention is supported by 76 libraries. The number of libraries was determined as previously described. This segment can be found in the following transcript(s): S67314_PEA_{—}1_T7 (SEQ ID NO:22). Table 15 below describes the starting and ending position of this segment on each transcript.

TABLE 15
Segment location on transcripts

| | Segment | Segment |
|---|---|---|
| | starting | ending |
| Transcript name | position | position |
| S67314_PEA_1_T7 (SEQ ID NO: 22) | 1306 | 3531 |

Segment cluster S67314_PEA_{—}1_node_{—}15 (SEQ ID NO:215) according to the present invention is supported by 1 libraries. The number of libraries was determined as previously described. This segment can be found in the following transcript(s): S67314_PEA_{—}1_T5 (SEQ ID NO:20). Table 16 below describes the starting and ending position of this segment on each transcript.

TABLE 16
Segment location on transcripts

| | Segment | Segment |
|---|---|---|
| | starting | ending |
| Transcript name | position | position |
| S67314_PEA_1_T5 (SEQ ID NO: 20) | 1273 | 1733 |

Segment cluster S67314_PEA_{—}1_node_{—}17 (SEQ ID NO:216) according to the present invention is supported by 4 libraries. The number of libraries was determined as previously described. This segment can be found in the following transcript(s): S67314_PEA_{—}1_T6 (SEQ ID NO:21). Table 17 below describes the starting and ending position of this segment on each transcript.

TABLE 17
Segment location on transcripts

| | Segment | Segment |
|---|---|---|
| | starting | ending |
| Transcript name | position | position |
| S67314_PEA_1_T6 (SEQ ID NO: 21) | 1273 | 1822 |

Microarray (chip) data is also available for this segment as follows. As described above with regard to the cluster itself, various oligonucleotides were tested for being differentially expressed in various disease conditions, particularly cancer. The following oligonucleotides were found to hit this segment with regard to colon cancer, shown in Table 18.

TABLE 18
Oligonucleotides related to this segment

| | Overexpressed | Chip |
|---|---|---|
| Oligonucleotide name | in cancers | reference |
| S67314_0_0_744 (SEQ ID NO: 1406) | colorectal cancer | Colon |

As a general note, oligonucleotide S67314_{—}0_{—}0_{—}741 (SEQ ID NO:1405) was overexpressed in colon cancer; this oligonucleotide maps to at least one part of this cluster.

Segment cluster S67314_PEA_{—}1_node_{—}4 (SEQ ID NO:217) according to the present invention is supported by 101 libraries. The number of libraries was determined as previously described. This segment can be found in the following transcript(s): S67314_PEA_{—}1_T4 (SEQ ID NO:19), S67314_PEA_{—}1_T5 (SEQ ID NO:20), S67314_PEA_{—}1_T6 (SEQ ID NO:21) and S67314_PEA_{—}1_T7 (SEQ ID NO:22). Table 19 below describes the starting and ending position of this segment on each transcript.

TABLE 19
Segment location on transcripts

| | Segment | Segment |
|---|---|---|
| | starting | ending |
| Transcript name | position | position |
| S67314_PEA_1_T4 (SEQ ID NO: 19) | 998 | 1170 |
| S67314_PEA_1_T5 (SEQ ID NO: 20) | 998 | 1170 |
| S67314_PEA_1_T6 (SEQ ID NO: 21) | 998 | 1170 |
| S67314_PEA_1_T7 (SEQ ID NO: 22) | 1031 | 1203 |

According to an optional embodiment of the present invention, short segments related to the above cluster are also provided. These segments are up to about 120 bp in length, and so are included in a separate description.

Segment cluster S67314_PEA_{—}1_node_{—}10 (SEQ ID NO:218) according to the present invention is supported by 64 libraries. The number of libraries was determined as previously described. This segment can be found in the following transcript(s): S67314_PEA_{—}1_T4 (SEQ ID NO:19), S67314_PEA_{—}1_T5 (SEQ ID NO:20), S67314_PEA_{—}1_T6 (SEQ ID NO:21) and S67314_PEA_{—}1_T7 (SEQ ID NO:22). Table 20 below describes the starting and ending position of this segment on each transcript.

TABLE 20
Segment location on transcripts

| | Segment | Segment |
|---|---|---|
| | starting | ending |
| Transcript name | position | position |
| S67314_PEA_1_T4 (SEQ ID NO: 19) | 1171 | 1272 |
| S67314_PEA_1_T5 (SEQ ID NO: 20) | 1171 | 1272 |
| S67314_PEA_1_T6 (SEQ ID NO: 21) | 1171 | 1272 |
| S67314_PEA_1_T7 (SEQ ID NO: 22) | 1204 | 1305 |

Segment cluster S67314_PEA_{—}1_node_{—}3 (SEQ ID NO:219) according to the present invention is supported by 1 libraries. The number of libraries was determined as previously described. This segment can be found in the following transcript(s): S67314_PEA_{—}1_T7 (SEQ ID NO:22). Table 21 below describes the starting and ending position of this segment on each transcript.

TABLE 21
Segment location on transcripts

| | Segment | Segment |
|---|---|---|
| | starting | ending |
| Transcript name | position | position |
| S67314_PEA_1_T7 (SEQ ID NO: 22) | 998 | 1030 |

### Variant Protein Alignment to the Previously Known Protein:

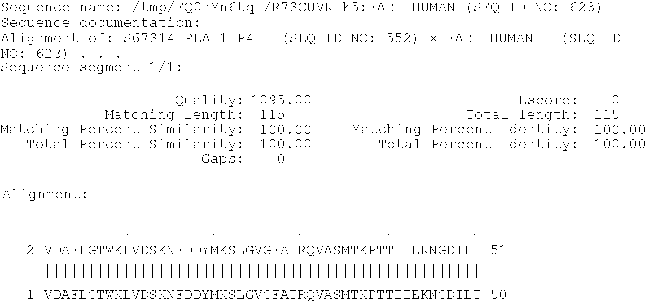

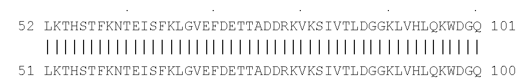

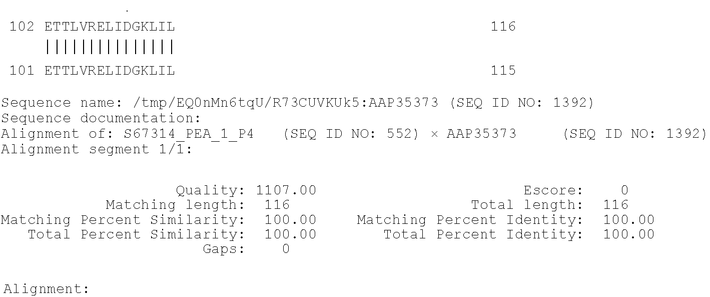

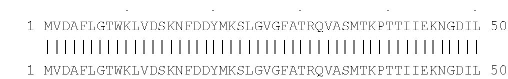

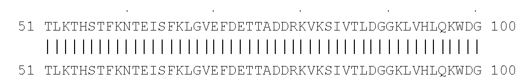

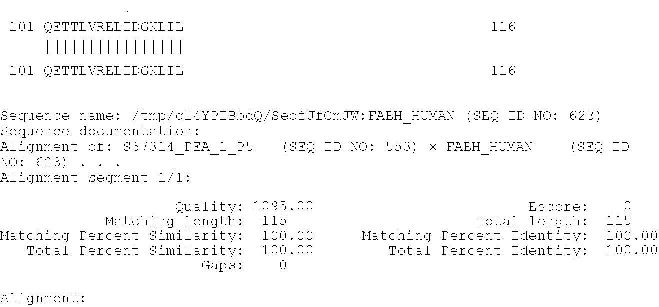

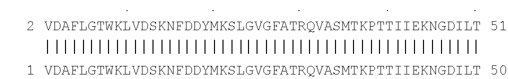

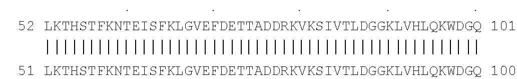

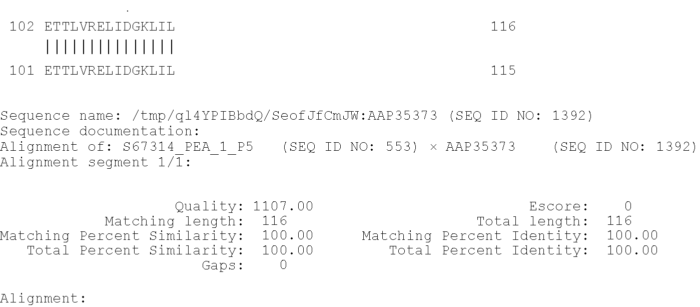

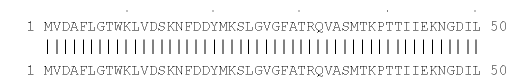

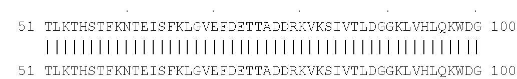

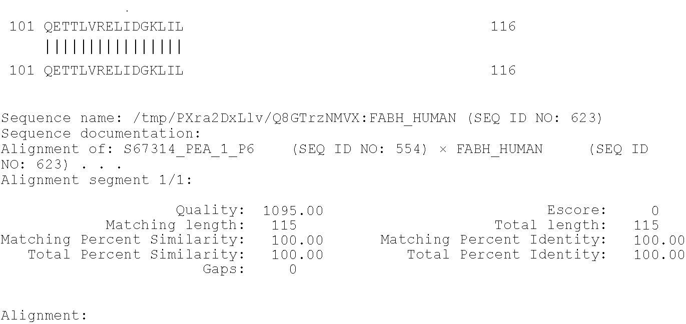

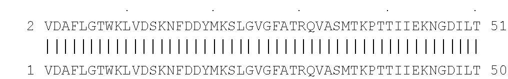

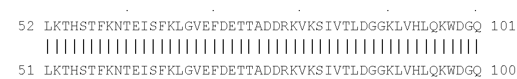

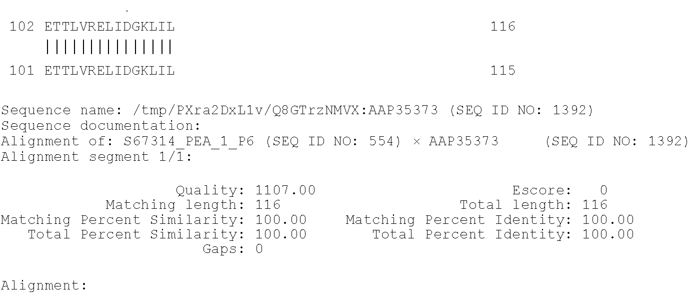

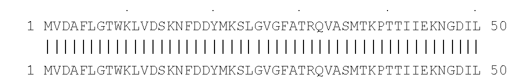

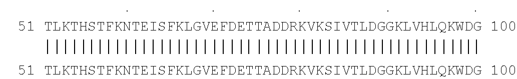

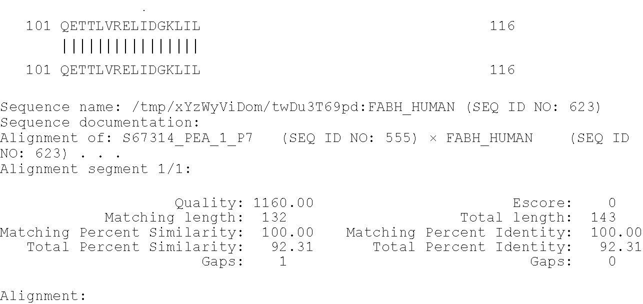

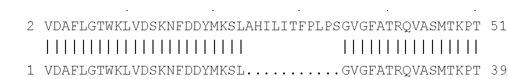

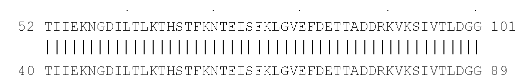

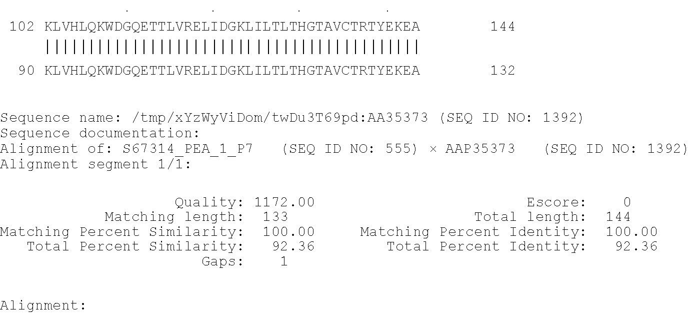

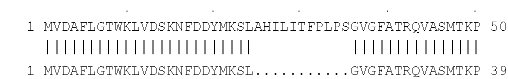

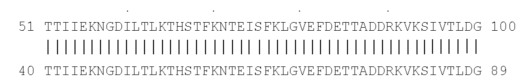

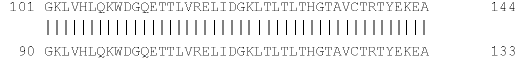

### Description for Cluster Z44808

Cluster Z44808 features 5 transcript(s) and 21 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| Z44808_PEA_1_T11 | 23 |
| Z44808_PEA_1_T4 | 24 |
| Z44808_PEA_1_T5 | 25 |
| Z44808_PEA_1_T8 | 26 |
| Z44808_PEA_1_T9 | 27 |

TABLE 2
Segments of interest

| Segment Name | SEQ ID NO: |
|---|---|
| Z44808_PEA_1_node_0 | 220 |
| Z44808_PEA_1_node_16 | 221 |
| Z44808_PEA_1_node_2 | 222 |
| Z44808_PEA_1_node_24 | 223 |
| Z44808_PEA_1_node_32 | 224 |
| Z44808_PEA_1_node_33 | 225 |
| Z44808_PEA_1_node_36 | 226 |
| Z44808_PEA_1_node_37 | 227 |
| Z44808_PEA_1_node_41 | 228 |
| Z44808_PEA_1_node_11 | 229 |
| Z44808_PEA_1_node_13 | 230 |
| Z44808_PEA_1_node_18 | 231 |
| Z44808_PEA_1_node_22 | 232 |
| Z44808_PEA_1_node_26 | 233 |
| Z44808_PEA_1_node_30 | 234 |
| Z44808_PEA_1_node_34 | 235 |
| Z44808_PEA_1_node_35 | 236 |
| Z44808_PEA_1_node_39 | 237 |
| Z44808_PEA_1_node_4 | 238 |
| Z44808_PEA_1_node_6 | 239 |
| Z44808_PEA_1_node_8 | 240 |

TABLE 3
Proteins of interest

| Protein Name | SEQ ID NO: |
|---|---|
| Z44808_PEA_1_P5 | 556 |
| Z44808_PEA_1_P6 | 557 |
| Z44808_PEA_1_P7 | 558 |
| Z44808_PEA_1_P11 | 559 |

These sequences are variants of the known protein SPARC related modular calcium-binding protein 2 precursor (SwissProt accession identifier SMO2_HUMAN; known also according to the synonyms Secreted modular calcium-binding protein 2; SMOC-2; Smooth muscle-associated protein 2; SMAP-2; MSTP117), SEQ ID NO: 624, referred to herein as the previously known protein.

Protein SPARC related modular calcium-binding protein 2 precursor is known or believed to have the following function(s): calcium binding . The sequence for protein SPARC related modular calcium-binding protein 2 precursor (SEQ ID NO:624) is given at the end of the application, as “SPARC related modular calcium-binding protein 2 precursor amino acid sequence”. Known polymorphisms for this sequence are as shown in Table 4.

TABLE 4
Amino acid mutations for Known Protein

| SNP position(s) on | |
|---|---|
| amino acid sequence | Comment |
| 169-170 | KT -> TR |
| 212 | S -> P |
| 429-466 | TPRGHAESTSNRQPRKQG -> RSKRNL |
| 434 | A -> V |
| 439 | N -> Y |

Protein SPARC related modular calcium-binding protein 2 precursor (SEQ ID NO:624) localization is believed to be Secreted (Probable).

Cluster Z44808 can be used as a diagnostic marker according to overexpression of transcripts of this cluster in cancer. Expression of such transcripts in normal tissues is also given according to the previously described methods. The term “number” in the right hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 19 and Table 5. This cluster is overexpressed (at least at a minimum level) in the following pathological conditions: colorectal cancer, lung cancer and pancreas carcinoma.

TABLE 5
Normal tissue distribution

| Name of Tissue | Number |
|---|---|
| bladder | 123 |
| bone | 304 |
| brain | 18 |
| colon | 0 |
| epithelial | 40 |
| general | 37 |
| kidney | 2 |
| lung | 0 |
| breast | 61 |
| ovary | 116 |
| pancreas | 0 |
| prostate | 128 |
| stomach | 36 |
| uterus | 195 |

TABLE 6
P values and ratios for expression in cancerous tissue

| Name of Tissue | P1 | P2 | SP1 | R3 | SP2 | R4 |
|---|---|---|---|---|---|---|
| bladder | 6.8e−01 | 7.6e−01 | 7.7e−01 | 0.8 | 9.1e−01 | 0.6 |
| bone | 7.0e−01 | 8.8e−01 | 9.9e−01 | 0.3 | 1 | 0.2 |
| brain | 6.8e−01 | 7.2e−01 | 3.0e−02 | 2.6 | 1.7e−01 | 1.6 |
| colon | 9.2e−03 | 1.3e−02 | 1.2e−01 | 3.6 | 1.6e−01 | 3.1 |
| epithelial | 2.1e−02 | 4.0e−01 | 1.0e−04 | 1.9 | 2.7e−01 | 1.0 |
| general | 2.6e−02 | 7.2e−01 | 4.9e−07 | 1.9 | 3.0e−01 | 1.0 |
| kidney | 7.3e−01 | 8.1e−01 | 1 | 1.0 | 1 | 1.0 |
| lung | 4.0e−03 | 1.8e−02 | 8.0e−04 | 12.2 | 2.1e−02 | 6.0 |
| breast | 4.8e−01 | 6.1e−01 | 9.8e−02 | 2.0 | 3.9e−01 | 1.2 |
| ovary | 8.1e−01 | 8.3e−01 | 9.1e−01 | 0.6 | 9.7e−01 | 0.5 |
| pancreas | 1.2e−01 | 2.1e−01 | 1.0e−03 | 6.5 | 5.9e−03 | 4.6 |
| prostate | 8.4e−01 | 8.9e−01 | 9.0e−01 | 0.6 | 9.8e−01 | 0.4 |
| stomach | 5.0e−01 | 8.7e−01 | 9.6e−04 | 1.5 | 1.9e−01 | 0.8 |
| uterus | 6.7e−01 | 7.9e−01 | 9.2e−01 | 0.5 | 1 | 0.3 |

As noted above, cluster Z44808 features 5 transcript(s), which were listed in Table 1 above. These transcript(s) encode for protein(s) which are variant(s) of protein SPARC related modular calcium-binding protein 2 precursor (SEQ ID NO:624). A description of each variant protein according to the present invention is now provided.

Variant protein Z44808_PEA_{—}1_P5 (SEQ ID NO:556) according to the present invention has an amino acid sequence as given at the end of the application; it is encoded by transcript(s) Z44808_PEA_{—}1_T4 (SEQ ID NO:24). An alignment is given to the known protein (SPARC related modular calcium-binding protein 2 precursor (SEQ ID NO:624)) at the end of the application. One or more alignments to one or more previously published protein sequences are given at the end of the application. A brief description of the relationship of the variant protein according to the present invention to each such aligned protein is as follows:

Comparison Report Between Z44808_PEA_{—}1_P5 (SEQ ID NO:556) and SMO2_HUMAN (SEQ ID NO:624):

1. An isolated chimeric polypeptide encoding for Z44808_PEA_{—}1_P5 (SEQ ID NO:556), comprising a first amino acid sequence being at least 90% homologous to MLLPQLCWLPLLAGLLPPVPAQKFSALTFLRVDQDKDKDCSLDCAGSPQKPLCASDGRTFLSRCEFQRAK CKDPQLEIAYRGNCKDVSRCVAERKYTQEQARKEFQQVFIPECNDDGTYSQVQCHSYTGYCWCVTPNGR PISGTAVAHKTPRCPGSVNEKLPQREGTGKTDDAAAPALETQPQGDEEDIASRYPTLWTEQVKSRQNKTN KNSVSSCDQEHQSALEEAKQPKNDNVVIPECAHGGLYKPVQCHPSTGYCWCVLVDTGRPIPGTSTRYEQP KCDNTARAHPAKARDLYKGRQLQGCPGAKKHEFLTSVLDALSTDMVHAASDPSSSSGRLSEPDPSHTLEE RVVHWYFKLLDKNSSGDIGKKEIKPFKRFLRKKSKPKKCVKKFVEYCDVNNDKSISVQELMGCLGVAKE DGKADTKKRHTPRGHAESTSNRQ corresponding to amino acids 1-441 of SMO2_HUMAN (SEQ ID NO:624), which also corresponds to amino acids 1-441 of Z44808_PEA_{—}1_P5 (SEQ ID NO:556), and a second amino acid sequence being at least 70%, optionally at least 80%, preferably at least 85%, more preferably at least 90% and most preferably at least 95% homologous to a polypeptide having the sequence DAMVVSSRPKATTHRKSRTLSRR (SEQ ID NO:1500) corresponding to amino acids 442-464 of Z44808_PEA_{—}1_P5 (SEQ ID NO:556), wherein said first and second amino acid sequences are contiguous and in a sequential order.

2. An isolated polypeptide encoding for a tail of Z44808_PEA_{—}1_P5 (SEQ ID NO:556), comprising a polypeptide being at least 70%, optionally at least about 80%, preferably at least about 85%, more preferably at least about 90% and most preferably at least about 95% homologous to the sequence DAMVVSSRPKATTHRKSRTLSRR (SEQ ID NO:1500) in Z44808_PEA_{—}1_P5 (SEQ ID NO:556).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein Z44808_PEA_{—}1_P5 (SEQ ID NO:556) is encoded by the following transcript(s): Z44808_PEA_{—}1_T4 (SEQ ID NO:24), for which the sequence(s) is/are given at the end of the application. The coding portion of transcript Z44808_PEA_{—}1_T4 (SEQ ID NO:24) is shown in bold; this coding portion starts at position 586 and ends at position 1977. The transcript also has the following SNPs as listed in Table 7 (given according to their position on the nucleotide sequence, with the alternative nucleic acid listed; the last column indicates whether the SNP is known or not; the presence of known SNPs in variant protein Z44808_PEA_{—}1_P5 (SEQ ID NO:556) sequence provides support for the deduced sequence of this variant protein according to the present invention).

TABLE 7
Nucleic acid SNPs

| SNP position on nucleotide | Alternative | Previously |
|---|---|---|
| sequence | nucleic acid | known SNP? |
| 549 | A -> G | No |
| 648 | T -> G | No |
| 4403 | G -> T | No |
| 4456 | G -> A | Yes |
| 4964 | G -> C | Yes |
| 1025 | C -> | No |
| 1677 | T -> C | No |
| 2691 | C -> T | Yes |
| 3900 | T -> C | No |
| 3929 | G -> A | Yes |
| 4099 | G -> T | Yes |
| 4281 | T -> C | No |
| 4319 | G -> C | Yes |

Variant protein Z44808_PEA_{—}1_P6 (SEQ ID NO:557) according to the present invention has an amino acid sequence as given at the end of the application; it is encoded by transcript(s) Z44808_PEA_{—}1_T5 (SEQ ID NO:25). An alignment is given to the known protein (SPARC related modular calcium-binding protein 2 precursor (SEQ ID NO:624)) at the end of the application. One or more alignments to one or more previously published protein sequences are given at the end of the application. A brief description of the relationship of the variant protein according to the present invention to each such aligned protein is as follows:

Comparison Report Between Z44808_PEA_{—}1_P6 (SEQ ID NO:557) and SMO2_HUMAN (SEQ ID NO:624):

1. An isolated chimeric polypeptide encoding for Z44808_PEA_{—}1_P6 (SEQ ID NO:557), comprising a first amino acid sequence being at least 90% homologous to MLLPQLCWLPLLAGLLPPVPAQKFSALTFLRVDQDKDKDCSLDCAGSPQKPLCASDGRTFLSRCEFQRAK CKDPQLEIAYRGNCKDVSRCVAERKYTQEQARKEFQQVFIPECNDDGTYSQVQCHSYTGYCWCVTPNGR PISGTAVAHKTPRCPGSVNEKLPQREGTGKTDDAAAPALETQPQGDEEDIASRYPTLWTEQVKSRQNKTN KNSVSSCDQEHQSALEEAKQPKNDNVVIPECAHGGLYKPVQCHPSTGYCWCVLVDTGRPIPGTSTRYEQP KCDNTARAHPAKARDLYKGRQLQGCPGAKKHEFLTSVLDALSTDMVHAASDPSSSSGRLSEPDPSHTLEE RVVHWYFKLLDKNSSGDIGKKEIKPFKRFLRKKSKPKKCVKKFVEYCDVNNDKSISVQELMGCLGVAKE DGKADTKKRH corresponding to amino acids 1-428 of SMO2_HUMAN (SEQ ID NO:624), which also corresponds to amino acids 1-428 of Z44808_PEA_{—}1_P6 (SEQ ID NO:557), and a second amino acid sequence being at least 70%, optionally at least 80%, preferably at least 85%, more preferably at least 90% and most preferably at least 95% homologous to a polypeptide having the sequence RSKRNL (SEQ ID NO:1501) corresponding to amino acids 1-428 of Z44808_PEA_{—}1_P6 (SEQ ID NO:557), wherein said first and second amino acid sequences are contiguous and in a sequential order.

2. An isolated polypeptide encoding for a tail of Z44808_PEA_{—}1_P6 (SEQ ID NO:557), comprising a polypeptide being at least 70%, optionally at least about 80%, preferably at least about 85%, more preferably at least about 90% and most preferably at least about 95% homologous to the sequence RSKRNL (SEQ ID NO:1501) in Z44808_PEA_{—}1_P6 (SEQ ID NO:557).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein Z44808_PEA_{—}1_P6 (SEQ ID NO:557) also has the following non-silent SNPs (Single Nucleotide Polymorphisms) as listed in Table 8, (given according to their position(s) on the amino acid sequence, with the alternative amino acid(s) listed; the last column indicates whether the SNP is known or not; the presence of known SNPs in variant protein Z44808_PEA_{—}1_P6 (SEQ ID NO:557) sequence provides support for the deduced sequence of this variant protein according to the present invention).

TABLE 8
Amino acid mutations

| SNP position(s) on amino acid | Alternative | Previously |
|---|---|---|
| sequence | amino acid(s) | known SNP? |
| 147 | A -> | No |

Variant protein Z44808_PEA_{—}1_P6 (SEQ ID NO:557) is encoded by the following transcript(s): Z44808_PEA_{—}1_T5 (SEQ ID NO:25), for which the sequence(s) is/are given at the end of the application. The coding portion of transcript Z44808_PEA_{—}1_T5 (SEQ ID NO:25) is shown in bold; this coding portion starts at position 586 and ends at position 1887. The transcript also has the following SNPs as listed in Table 9 (given according to their position on the nucleotide sequence, with the alternative nucleic acid listed; the last column indicates whether the SNP is known or not; the presence of known SNPs in variant protein Z44808_PEA_{—}1_P6 (SEQ ID NO:557) sequence provides support for the deduced sequence of this variant protein according to the present invention).

TABLE 9
Nucleic acid SNPs

| SNP position on nucleotide | Alternative | Previously |
|---|---|---|
| sequence | nucleic acid | known SNP? |
| 549 | A -> G | No |
| 648 | T -> G | No |
| 2866 | G -> A | Yes |
| 3374 | G -> C | Yes |
| 1025 | C -> | No |
| 1677 | T -> C | No |
| 2310 | T -> C | No |
| 2339 | G -> A | Yes |
| 2509 | G -> T | Yes |
| 2691 | T -> C | No |
| 2729 | G -> C | Yes |
| 2813 | G -> T | No |

Variant protein Z44808_PEA_{—}1_P7 (SEQ ID NO:558) according to the present invention has an amino acid sequence as given at the end of the application; it is encoded by transcript(s) Z44808_PEA_{—}1_T9 (SEQ ID NO:27). An alignment is given to the known protein (SPARC related modular calcium-binding protein 2 precursor (SEQ ID NO:624)) at the end of the application. One or more alignments to one or more previously published protein sequences are given at the end of the application. A brief description of the relationship of the variant protein according to the present invention to each such aligned protein is as follows:

Comparison Report Between Z44808_PEA_{—}1_P7 (SEQ ID NO:558) and SMO2_HUMAN (SEQ ID NO:624):

1. An isolated chimeric polypeptide encoding for Z44808_PEA_{—}1_P7 (SEQ ID NO:558), comprising a first amino acid sequence being at least 90% homologous to MLLPQLCWLPLLAGLLPPVPAQKFSALTFLRVDQDKDKDCSLDCAGSPQKPLCASDGRTFLSRCEFQRAK CKDPQLEIAYRGNCKDVSRCVAERKYTQEQARKEFQQVFIPECNDDGTYSQVQCHSYTGYCWCVTPNGR PISGTAVAHKTPRCPGSVNEKLPQREGTGKTDDAAAPALETQPQGDEEDIASRYPTLWTEQVKSRQNKTN KNSVSSCDQEHQSALEEAKQPKNDNVVIPECAHGGLYKPVQCHPSTGYCWCVLVDTGRPIPGTSTRYEQP KCDNTARAHPAKARDLYKGRQLQGCPGAKKHEFLTSVLDALSTDMVHAASDPSSSSGRLSEPDPSHTLEE RVVHWYFKLLDKNSSGDIGKKEIKPFKRFLRKKSKPKKCVKKFVEYCDVNNDKSISVQELMGCLGVAKE DGKADTKKRHTPRGHAESTSNRQ corresponding to amino acids 1-441 of SMO2_HUMAN (SEQ ID NO:624), which also corresponds to amino acids 1-441 of Z44808_PEA_{—}1_P7 (SEQ ID NO:558), and a second amino acid sequence being at least 70%, optionally at least 80%, preferably at least 85%, more preferably at least 90% and most preferably at least 95% homologous to a polypeptide having the sequence LLWLRGKVSFYCF (SEQ ID NO:1502) corresponding to amino acids 442-454 of Z44808_PEA_{—}1_P7 (SEQ ID NO:558), wherein said first and second amino acid sequences are contiguous and in a sequential order.

2. An isolated polypeptide encoding for a tail of Z44808_PEA_{—}1_P7 (SEQ ID NO:558), comprising a polypeptide being at least 70%, optionally at least about 80%, preferably at least about 85%, more preferably at least about 90% and most preferably at least about 95% homologous to the sequence LLWLRGKVSFYCF (SEQ ID NO:1502) in Z44808_PEA_{—}1_P7 (SEQ ID NO:558).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein Z44808_PEA_{—}1_P7 (SEQ ID NO:558) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein Z44808_PEA_{—}1_P7 (SEQ ID NO:558) sequence provides support for the deduced sequence of this variant protein according to the present invention).

TABLE 10
Amino acid mutations

| SNP position(s) on amino acid | Alternative | Previously |
|---|---|---|
| sequence | amino acid(s) | known SNP? |
| 147 | A -> | No |

Variant protein Z44808_PEA_{—}1_P7 (SEQ ID NO:558) is encoded by the following transcript(s): Z44808_PEA_{—}1_T9 (SEQ ID NO:27), for which the sequence(s) is/are given at the end of the application. The coding portion of transcript Z44808_PEA_{—}1_T9 (SEQ ID NO:27) is shown in bold; this coding portion starts at position 586 and ends at position 1947. The transcript also has the following SNPs as listed in Table 11 (given according to their position on the nucleotide sequence, with the alternative nucleic acid listed; the last column indicates whether the SNP is known or not; the presence of known SNPs in variant protein Z44808_PEA_{—}1_P7 (SEQ ID NO:558) sequence provides support for the deduced sequence of this variant protein according to the present invention).

TABLE 11
Nucleic acid SNPs

| SNP position on nucleotide | Alternative | Previously |
|---|---|---|
| sequence | nucleic acid | known SNP? |
| 549 | A -> G | No |
| 648 | T -> G | No |
| 1025 | C -> | No |
| 1677 | T -> C | No |
| 2169 | C -> A | Yes |

Variant protein Z44808_PEA_{—}1_P11 (SEQ ID NO:559) according to the present invention has an amino acid sequence as given at the end of the application; it is encoded by transcript(s) Z44808_PEA_{—}1_T11 (SEQ ID NO:23). The identification of this transcript was performed using a non-EST based method for identification of alternative splicing, described in the following reference: “Sorek R et al., Genome Res. (2004) 14:1617-23.” An alignment is given to the known protein (SPARC related modular calcium-binding protein 2 precursor (SEQ ID NO:624)) at the end of the application. One or more alignments to one or more previously published protein sequences are given at the end of the application. A brief description of the relationship of the variant protein according to the present invention to each such aligned protein is as follows:

Comparison Report Between Z44808_PEA_{—}1_P11 (SEQ ID NO:559) and SMO2_HUMAN (SEQ ID NO:624):

1. An isolated chimeric polypeptide encoding for Z44808_PEA_{—}1_P11 (SEQ ID NO:559), comprising a first amino acid sequence being at least 90% homologous to MLLPQLCWLPLLAGLLPPVPAQKFSALTFLRVDQDKDKDCSLDCAGSPQKPLCASDGRTFLSRCEFQRAK CKDPQLEIAYRGNCKDVSRCVAERKYTQEQARKEFQQVFIPECNDDGTYSQVQCHSYTGYCWCVTPNGR PISGTAVAHKTPRCPGSVNEKLPQREGTGKT corresponding to amino acids 1-170 of SMO2_HUMAN (SEQ ID NO:624), which also corresponds to amino acids 1-170 of Z44808_PEA_{—}1_P11 (SEQ ID NO:559), and a second amino acid sequence being at least 90% homologous to DIASRYPTLWTEQVKSRQNKTNKNSVSSCDQEHQSALEEAKQPKNDNVVIPECAHGGLYKPVQCHPSTGY CWCVLVDTGRPIPGTSTRYEQPKCDNTARAHPAKARDLYKGRQLQGCPGAKKHEFLTSVLDALSTDMVH AASDPSSSSGRLSEPDPSHTLEERVVHWYFKLLDKNSSGDIGKKEIKPFKRFLRKKSKPKKCVKKFVEYCD VNNDKSISVQELMGCLGVAKEDGKADTKKRHTPRGHAESTSNRQPRKQG corresponding to amino acids 188-446 of SMO2_HUMAN (SEQ ID NO:624), which also corresponds to amino acids 171-429 of Z44808_PEA_{—}1_P11 (SEQ ID NO:559), wherein said first and second amino acid sequences are contiguous and in a sequential order.

2. An isolated chimeric polypeptide encoding for an edge portion of Z44808_PEA_{—}1_P11 (SEQ ID NO:559) comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise TD, having a structure as follows: a sequence starting from any of amino acid numbers 170−x to 170; and ending at any of amino acid numbers 171+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein Z44808_PEA_{—}1_P11 (SEQ ID NO:559) also has the following non-silent SNPs (Single Nucleotide Polymorphisms) as listed in Table 12, (given according to their position(s) on the amino acid sequence, with the alternative amino acid(s) listed; the last column indicates whether the SNP is known or not; the presence of known SNPs in variant protein Z44808_PEA_{—}1_P11 (SEQ ID NO:559) sequence provides support for the deduced sequence of this variant protein according to the present invention).

TABLE 12
Amino acid mutations

| SNP position(s) on amino acid | Alternative | Previously |
|---|---|---|
| sequence | amino acid(s) | known SNP? |
| 147 | A -> | No |

Variant protein Z44808_PEA_{—}1_P11 (SEQ ID NO:559) is encoded by the following transcript(s): Z44808_PEA_{—}1_T11 (SEQ ID NO:23), for which the sequence(s) is/are given at the end of the application. The coding portion of transcript Z44808_PEA_{—}1_T11 (SEQ ID NO:23) is shown in bold; this coding portion starts at position 586 and ends at position 1872. The transcript also has the following SNPs as listed in Table 13 (given according to their position on the nucleotide sequence, with the alternative nucleic acid listed; the last column indicates whether the SNP is known or not; the presence of known SNPs in variant protein Z44808_PEA_{—}1_P11 (SEQ ID NO:559) sequence provides support for the deduced sequence of this variant protein according to the present invention).

TABLE 13
Nucleic acid SNPs

| SNP position on | Alternative | Previously |
|---|---|---|
| nucleotide sequence | nucleic acid | known SNP? |
| 549 | A -> G | No |
| 648 | T -> G | No |
| 2720 | G -> A | Yes |
| 3228 | G -> C | Yes |
| 1025 | C -> | No |
| 1626 | T -> C | No |
| 2164 | T -> C | No |
| 2193 | G -> A | Yes |
| 2363 | G -> T | Yes |
| 2545 | T -> C | No |
| 2583 | G -> C | Yes |
| 2667 | G -> T | No |

As noted above, cluster Z44808 features 21 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster Z44808_PEA_{—}1_node_{—}0 (SEQ ID NO:220) according to the present invention is supported by 29 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24), Z44808_PEA_{—}1_T5 (SEQ ID NO:25), Z44808_PEA_{—}1_T8 (SEQ ID NO:26) and Z44808_PEA_{—}1_T9 (SEQ ID NO:27). Table 14 below describes the starting and ending position of this segment on each transcript.

TABLE 14
Segment location on transcripts

| | Segment | |
|---|---|---|
| | starting | Segment |
| Transcript name | position | ending position |
| Z44808_PEA_1_T11 (SEQ ID NO: 23) | 1 | 669 |
| Z44808_PEA_1_T4 (SEQ ID NO: 24) | 1 | 669 |
| Z44808_PEA_1_T5 (SEQ ID NO: 25) | 1 | 669 |
| Z44808_PEA_1_T8 (SEQ ID NO: 26) | 1 | 669 |
| Z44808_PEA_1_T9 (SEQ ID NO: 27) | 1 | 669 |

Segment cluster Z44808_PEA_{—}1_node_{—}16 (SEQ ID NO:221) according to the present invention is supported by 39 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24), Z44808_PEA_{—}1_T5 (SEQ ID NO:25), Z44808_PEA_{—}1_T8 (SEQ ID NO:26) and Z44808_PEA_{—}1_T9 (SEQ ID NO:27). Table 15 below describes the starting and ending position of this segment on each transcript.

TABLE 15
Segment location on transcripts

| | Segment | |
|---|---|---|
| | starting | Segment |
| Transcript name | position | ending position |
| Z44808_PEA_1_T11 (SEQ ID NO: 23) | 1172 | 1358 |
| Z44808_PEA_1_T4 (SEQ ID NO: 24) | 1223 | 1409 |
| Z44808_PEA_1_T5 (SEQ ID NO: 25) | 1223 | 1409 |
| Z44808_PEA_1_T8 (SEQ ID NO: 26) | 1223 | 1409 |
| Z44808_PEA_1_T9 (SEQ ID NO: 27) | 1223 | 1409 |

Segment cluster Z44808_PEA_{—}1_node_{—}2 (SEQ ID NO:222) according to the present invention is supported by 34 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24), Z44808_PEA_{—}1_T5 (SEQ ID NO:25), Z44808_PEA_{—}1_T8 (SEQ ID NO:26) and Z44808_PEA_{—}1_T9 (SEQ ID NO:27). Table 16 below describes the starting and ending position of this segment on each transcript.

TABLE 16
Segment location on transcripts

| | Segment | |
|---|---|---|
| | starting | Segment |
| Transcript name | position | ending position |
| Z44808_PEA_1_T11 (SEQ ID NO: 23) | 670 | 841 |
| Z44808_PEA_1_T4 (SEQ ID NO: 24) | 670 | 841 |
| Z44808_PEA_1_T5 (SEQ ID NO: 25) | 670 | 841 |
| Z44808_PEA_1_T8 (SEQ ID NO: 26) | 670 | 841 |
| Z44808_PEA_1_T9 (SEQ ID NO: 27) | 670 | 841 |

Segment cluster Z44808_PEA_{—}1_node_{—}24 (SEQ ID NO:223) according to the present invention is supported by 52 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24), Z44808_PEA_{—}1_T5 (SEQ ID NO:25), Z44808_PEA_{—}1_T8 (SEQ ID NO:26) and Z44808_PEA_{—}1_T9 (SEQ ID NO:27). Table 17 below describes the starting and ending position of this segment on each transcript.

TABLE 17
Segment location on transcripts

| | Segment | |
|---|---|---|
| | starting | Segment |
| Transcript name | position | ending position |
| Z44808_PEA_1_T11 (SEQ ID NO: 23) | 1545 | 1819 |
| Z44808_PEA_1_T4 (SEQ ID NO: 24) | 1596 | 1870 |
| Z44808_PEA_1_T5 (SEQ ID NO: 25) | 1596 | 1870 |
| Z44808_PEA_1_T8 (SEQ ID NO: 26) | 1596 | 1870 |
| Z44808_PEA_1_T9 (SEQ ID NO: 27) | 1596 | 1870 |

Segment cluster Z44808_PEA_{—}1_node_{—}32 (SEQ ID NO:224) according to the present invention is supported by 17 libraries. The number of libraries was determined as previously described. This segment can be found in the following transcript(s): Z44808_PEA_{—}1_T4 (SEQ ID NO:24) and Z44808_PEA_{—}1_T8 (SEQ ID NO:26). Table 18 below describes the starting and ending position of this segment on each transcript.

TABLE 18
Segment location on transcripts

| | Segment | |
|---|---|---|
| | starting | Segment |
| Transcript name | position | ending position |
| Z44808_PEA_1_T4 (SEQ ID NO: 24) | 1909 | 3593 |
| Z44808_PEA_1_T8 (SEQ ID NO: 26) | 1909 | 2397 |

Segment cluster Z44808_PEA_{—}1_node_{—}33 (SEQ ID NO:225) according to the present invention is supported by 133 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24) and Z44808_PEA_{—}1_T5 (SEQ ID NO:25). Table 20 below describes the starting and ending position of this segment on each transcript.

TABLE 20
Segment location on transcripts

| | Segment | |
|---|---|---|
| | starting | Segment |
| Transcript name | position | ending position |
| Z44808_PEA_1_T11 (SEQ ID NO: 23) | 1858 | 2734 |
| Z44808_PEA_1_T4 (SEQ ID NO: 24) | 3594 | 4470 |
| Z44808_PEA_1_T5 (SEQ ID NO: 25) | 2004 | 2880 |

Segment cluster Z44808_PEA_{—}1_node_{—}36 (SEQ ID NO:226) according to the present invention is supported by 117 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24) and Z44808_PEA_{—}1_T5 (SEQ ID NO:25). Table 21 below describes the starting and ending position of this segment on each transcript.

TABLE 21
Segment location on transcripts

| | Segment | |
|---|---|---|
| | starting | Segment |
| Transcript name | position | ending position |
| Z44808_PEA_1_T11 (SEQ ID NO: 23) | 2829 | 3080 |
| Z44808_PEA_1_T4 (SEQ ID NO: 24) | 4565 | 4816 |
| Z44808_PEA_1_T5 (SEQ ID NO: 25) | 2975 | 3226 |

Segment cluster Z44808_PEA_{—}1_node_{—}37 (SEQ ID NO:227) according to the present invention is supported by 120 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24) and Z44808_PEA_{—}1_T5 (SEQ ID NO:25). Table 22 below describes the starting and ending position of this segment on each transcript.

TABLE 22
Segment location on transcripts

| | Segment | |
|---|---|---|
| | starting | Segment |
| Transcript name | position | ending position |
| Z44808_PEA_1_T11 (SEQ ID NO: 23) | 3081 | 3429 |
| Z44808_PEA_1_T4 (SEQ ID NO: 24) | 4817 | 5165 |
| Z44808_PEA_1_T5 (SEQ ID NO: 25) | 3227 | 3575 |

Segment cluster Z44808_PEA_{—}1_node_{—}41 (SEQ ID NO:228) according to the present invention is supported by 2 libraries. The number of libraries was determined as previously described. This segment can be found in the following transcript(s): Z44808_PEA_{—}1_T9 (SEQ ID NO:27). Table 23 below describes the starting and ending position of this segment on each transcript.

TABLE 23
Segment location on transcripts

| | Segment | |
|---|---|---|
| | starting | Segment |
| Transcript name | position | ending position |
| Z44808_PEA_1_T9 (SEQ ID NO: 27) | 1974 | 2206 |

According to an optional embodiment of the present invention, short segments related to the above cluster are also provided. These segments are up to about 120 bp in length, and so are included in a separate description.

Segment cluster Z44808_PEA_{—}1_node_{—}11 (SEQ ID NO:229) according to the present invention is supported by 25 libraries. The number of libraries was determined as previously described. This segment can be found in the following transcript(s): Z44808_PEA_{—}1_T4 (SEQ ID NO:24), Z44808_PEA_{—}1_T5 (SEQ ID NO:25), Z44808_PEA_{—}1_T8 (SEQ ID NO:26) and Z44808_PEA_{—}1_T9 (SEQ ID NO:27). Table 24 below describes the starting and ending position of this segment on each transcript.

TABLE 24
Segment location on transcripts

| | Segment | |
|---|---|---|
| | starting | Segment |
| Transcript name | position | ending position |
| Z44808_PEA_1_T4 (SEQ ID NO: 24) | 1097 | 1147 |
| Z44808_PEA_1_T5 (SEQ ID NO: 25) | 1097 | 1147 |
| Z44808_PEA_1_T8 (SEQ ID NO: 26) | 1097 | 1147 |
| Z44808_PEA_1_T9 (SEQ ID NO: 27) | 1097 | 1147 |

Segment cluster Z44808_PEA_{—}1_node_{—}13 (SEQ ID NO:230) according to the present invention is supported by 28 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24), Z44808_PEA_{—}1_T5 (SEQ ID NO:25), Z44808_PEA_{—}1_T8 (SEQ ID NO:26) and Z44808_PEA_{—}1_T9 (SEQ ID NO:27). Table 25 below describes the starting and ending position of this segment on each transcript.

TABLE 25
Segment location on transcripts

| | Segment | |
|---|---|---|
| | starting | Segment |
| Transcript name | position | ending position |
| Z44808_PEA_1_T11 (SEQ ID NO: 23) | 1097 | 1171 |
| Z44808_PEA_1_T4 (SEQ ID NO: 24) | 1148 | 1222 |
| Z44808_PEA_1_T5 (SEQ ID NO: 25) | 1148 | 1222 |
| Z44808_PEA_1_T8 (SEQ ID NO: 26) | 1148 | 1222 |
| Z44808_PEA_1_T9 (SEQ ID NO: 27) | 1148 | 1222 |

Segment cluster Z44808_PEA_{—}1_node_{—}18 (SEQ ID NO:231) according to the present invention is supported by 27 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24), Z44808_PEA_{—}1_T5 (SEQ ID NO:25), Z44808_PEA_{—}1_T8 (SEQ ID NO:26) and Z44808_PEA_{—}1_T9 (SEQ ID NO:27). Table 26 below describes the starting and ending position of this segment on each transcript.

TABLE 26
Segment location on transcripts

| | Segment | |
|---|---|---|
| | starting | Segment |
| Transcript name | position | ending position |
| Z44808_PEA_1_T11 (SEQ ID NO: 23) | 1359 | 1441 |
| Z44808_PEA_1_T4 (SEQ ID NO: 24) | 1410 | 1492 |
| Z44808_PEA_1_T5 (SEQ ID NO: 25) | 1410 | 1492 |
| Z44808_PEA_1_T8 (SEQ ID NO: 26) | 1410 | 1492 |
| Z44808_PEA_1_T9 (SEQ ID NO: 27) | 1410 | 1492 |

Segment cluster Z44808_PEA_{—}1_node_{—}22 (SEQ ID NO:232) according to the present invention is supported by 33 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24), Z44808_PEA_{—}1_T5 (SEQ ID NO:25), Z44808_PEA_{—}1_T8 (SEQ ID NO:26) and Z44808_PEA_{—}1_T9 (SEQ ID NO:27). Table 27 below describes the starting and ending position of this segment on each transcript.

TABLE 27
Segment location on transcripts

| | Segment | |
|---|---|---|
| | starting | Segment |
| Transcript name | position | ending position |
| Z44808_PEA_1_T11 (SEQ ID NO: 23) | 1442 | 1544 |
| Z44808_PEA_1_T4 (SEQ ID NO: 24) | 1493 | 1595 |
| Z44808_PEA_1_T5 (SEQ ID NO: 25) | 1493 | 1595 |
| Z44808_PEA_1_T8 (SEQ ID NO: 26) | 1493 | 1595 |
| Z44808_PEA_1_T9 (SEQ ID NO: 27) | 1493 | 1595 |

Microarray (chip) data is also available for this segment as follows. As described above with regard to the cluster itself, various oligonucleotides were tested for being differentially expressed in various disease conditions, particularly cancer. The following oligonucleotides were found to hit this segment, shown in Table 28.

TABLE 28
Oligonucleotides related to this segment

| | Overexpressed | |
|---|---|---|
| Oligonucleotide name | in cancers | Chip reference |
| Z44808_0_8_0 (SEQ ID NO: 1407) | Lung squamous | LUN |
| | cell carcinoma | |

Segment cluster Z44808_PEA_{—}1_node_{—}26 (SEQ ID NO:233) according to the present invention is supported by 2 libraries. The number of libraries was determined as previously described. This segment can be found in the following transcript(s): Z44808_PEA_{—}1_T5 (SEQ ID NO:25). Table 29 below describes the starting and ending position of this segment on each transcript.

TABLE 29
Segment location on transcripts

| | | Segment |
|---|---|---|
| | Segment | ending |
| Transcript name | starting position | position |
| Z44808_PEA_1_T5 (SEQ ID NO: 25) | 1871 | 1965 |

Segment cluster Z44808_PEA_{—}1_node_{—}30 (SEQ ID NO:234) according to the present invention is supported by 44 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24), Z44808_PEA_{—}1_T5 (SEQ ID NO:25), Z44808_PEA_{—}1_T8 (SEQ ID NO:26) and Z44808_PEA_{—}1_T9 (SEQ ID NO:27). Table 31 below describes the starting and ending position of this segment on each transcript.

TABLE 31
Segment location on transcripts

| | | Segment |
|---|---|---|
| | Segment | ending |
| Transcript name | starting position | position |
| Z44808_PEA_1_T11 (SEQ ID NO: 23) | 1820 | 1857 |
| Z44808_PEA_1_T4 (SEQ ID NO: 24) | 1871 | 1908 |
| Z44808_PEA_1_T5 (SEQ ID NO: 25) | 1966 | 2003 |
| Z44808_PEA_1_T8 (SEQ ID NO: 26) | 1871 | 1908 |
| Z44808_PEA_1_T9 (SEQ ID NO: 27) | 1871 | 1908 |

Segment cluster Z44808_PEA_{—}1_node_{—}34 (SEQ ID NO:235) according to the present invention is supported by 70 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24) and Z44808_PEA_{—}1_T5 (SEQ ID NO:25). Table 32 below describes the starting and ending position of this segment on each transcript.

TABLE 32
Segment location on transcripts

| | | Segment |
|---|---|---|
| | Segment | ending |
| Transcript name | starting position | position |
| Z44808_PEA_1_T11 (SEQ ID NO: 23) | 2735 | 2809 |
| Z44808_PEA_1_T4 (SEQ ID NO: 24) | 4471 | 4545 |
| Z44808_PEA_1_T5 (SEQ ID NO: 25) | 2881 | 2955 |

Segment cluster Z44808_PEA_{—}1_node_{—}35 (SEQ ID NO:236) according to the present invention can be found in the following transcript(s): Z44808_PEA_{—}1_T11 (SEQ ID NO:23), Z44808_PEA_{—}1_T4 (SEQ ID NO:24) and Z44808_PEA_{—}1_T5 (SEQ ID NO:25). Table 33 below describes the starting and ending position of this segment on each transcript.

TABLE 33
Segment location on transcripts

| | | Segment |
|---|---|---|
| | Segment | ending |
| Transcript name | starting position | position |
| Z44808_PEA_1_T11 (SEQ ID NO: 23) | 2810 | 2828 |
| Z44808_PEA_1_T4 (SEQ ID NO: 24) | 4546 | 4564 |
| Z44808_PEA_1_T5 (SEQ ID NO: 25) | 2956 | 2974 |

Segment cluster Z44808_PEA_{—}1_node_{—}39 (SEQ ID NO:237) according to the present invention is supported by 1 libraries. The number of libraries was determined as previously described. This segment can be found in the following transcript(s): Z44808_PEA_{—}1_T9 (SEQ ID NO:27). Table 34 below describes the starting and ending position of this segment on each transcript.

TABLE 34
Segment location on transcripts

| | | Segment |
|---|---|---|
| | Segment | ending |
| Transcript name | starting position | position |
| Z44808_PEA_1_T9 (SEQ ID NO: 27) | 1909 | 1973 |

Segment cluster Z44808_PEA_{—}1_node_{—}4 (SEQ ID NO:238) according to the present invention is supported by 33 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24), Z44808_PEA_{—}1_T5 (SEQ ID NO:25) Z44808_PEA_{—}1_T8 (SEQ ID NO:26) and Z44808_PEA_{—}1_T9 (SEQ ID NO:27). Table 35 below describes the starting and ending position of this segment on each transcript.

TABLE 35
Segment location on transcripts

| | | Segment |
|---|---|---|
| | Segment | ending |
| Transcript name | starting position | position |
| Z44808_PEA_1_T11 (SEQ ID NO: 23) | 842 | 948 |
| Z44808_PEA_1_T4 (SEQ ID NO: 24) | 842 | 948 |
| Z44808_PEA_1_T5 (SEQ ID NO: 25) | 842 | 948 |
| Z44808_PEA_1_T8 (SEQ ID NO: 26) | 842 | 948 |
| Z44808_PEA_1_T9 (SEQ ID NO: 27) | 842 | 948 |

Segment cluster Z44808_PEA_{—}1_node_{—}6 (SEQ ID NO:239) according to the present invention is supported by 30 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24), Z44808_PEA_{—}1_T5 (SEQ ID NO:25), Z44808_PEA_{—}1_T8 (SEQ ID NO:26) and Z44808_PEA_{—}1_T9 (SEQ ID NO:27). Table 36 below describes the starting and ending position of this segment on each transcript.

TABLE 36
Segment location on transcripts

| | | Segment |
|---|---|---|
| | Segment | ending |
| Transcript name | starting position | position |
| Z44808_PEA_1_T11 (SEQ ID NO: 23) | 949 | 1048 |
| Z44808_PEA_1_T4 (SEQ ID NO: 24) | 949 | 1048 |
| Z44808_PEA_1_T5 (SEQ ID NO: 25) | 949 | 1048 |
| Z44808_PEA_1_T8 (SEQ ID NO: 26) | 949 | 1048 |
| Z44808_PEA_1_T9 (SEQ ID NO: 27) | 949 | 1048 |

Segment cluster Z44808_PEA_{—}1_node_{—}8 (SEQ ID NO:240) according to the present invention is supported by 25 libraries. The number of libraries was determined as previously described. This segment can be found in the following transcript(s): Z44808_PEA_{—}1_T11 (SEQ ID NO:23), Z44808_PEA_{—}1_T4 (SEQ ID NO:24), Z44808_PEA_{—}1_T5 (SEQ ID NO:25), Z44808_PEA_{—}1_T8 (SEQ ID NO:26) and Z44808_PEA_{—}1_T9 (SEQ ID NO:27). Table 37 below describes the starting and ending position of this segment on each transcript.

TABLE 37
Segment location on transcripts

| | | Segment |
|---|---|---|
| | Segment | ending |
| Transcript name | starting position | position |
| Z44808_PEA_1_T11 (SEQ ID NO: 23) | 1049 | 1096 |
| Z44808_PEA_1_T4 (SEQ ID NO: 24) | 1049 | 1096 |
| Z44808_PEA_1_T5 (SEQ ID NO: 25) | 1049 | 1096 |
| Z44808_PEA_1_T8 (SEQ ID NO: 26) | 1049 | 1096 |
| Z44808_PEA_1_T9 (SEQ ID NO: 27) | 1049 | 1096 |

### Variant Protein Alignment to the Previously Known Protein:

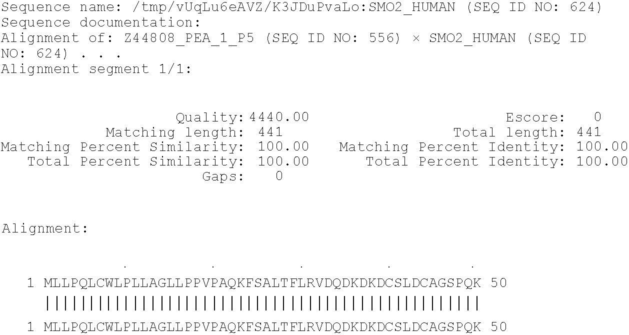

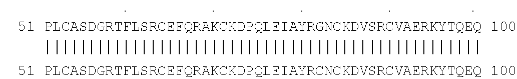

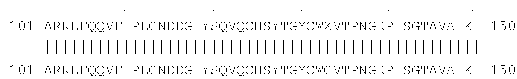

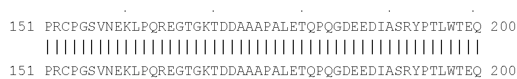

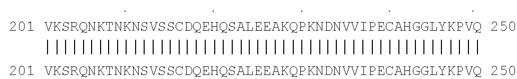

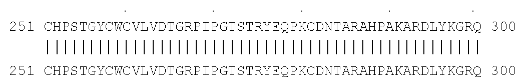

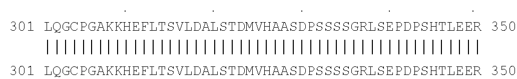

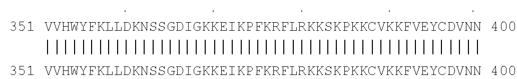

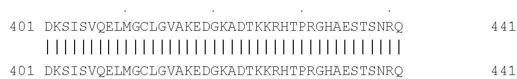

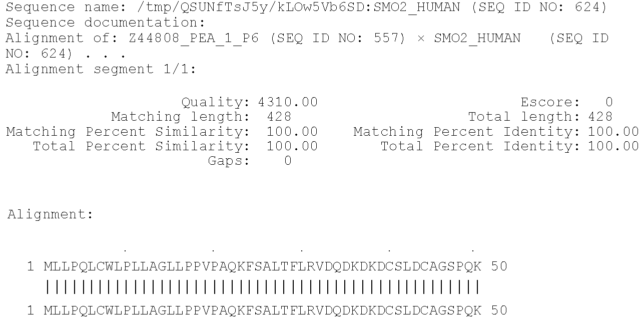

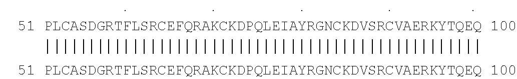

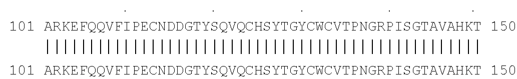

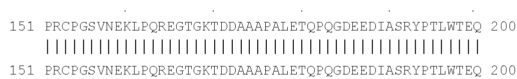

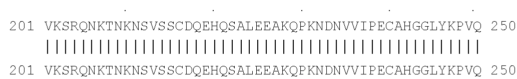

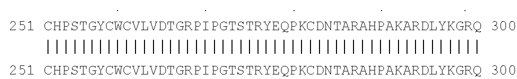

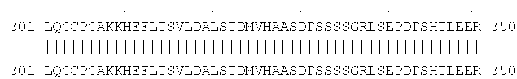

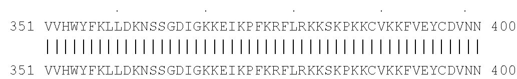

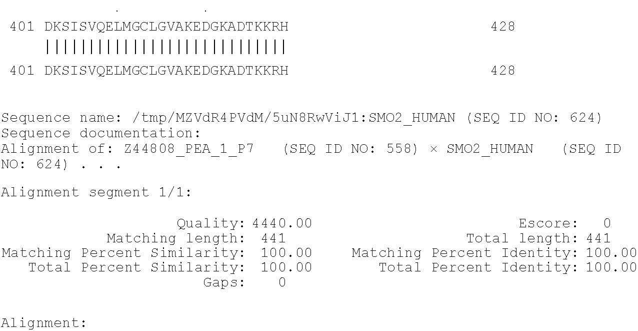

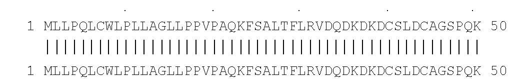

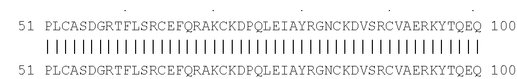

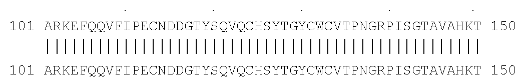

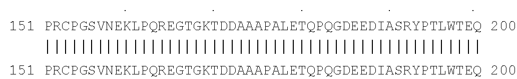

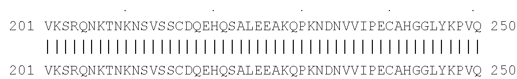

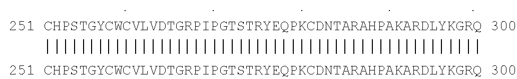

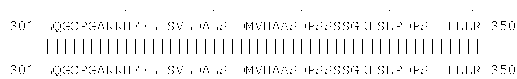

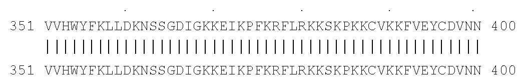

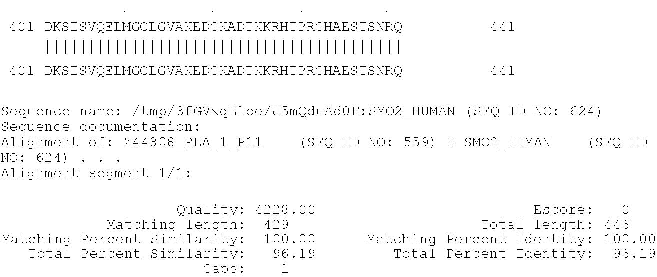

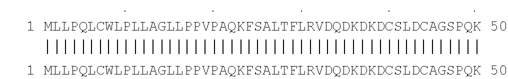

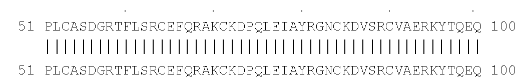

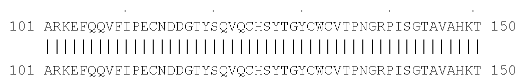

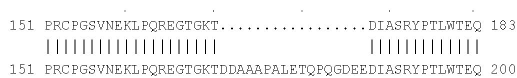

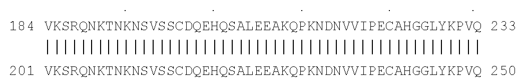

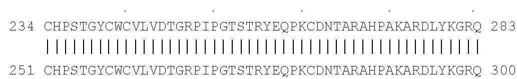

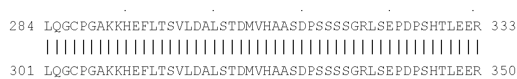

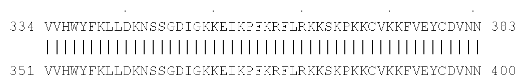

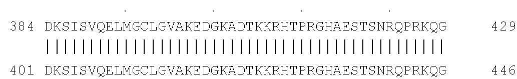

### Expression of SMO2_HUMAN SPARC Related Modular Calcium-Binding Protein 2 Precursor (Secreted Modular Calcium-Binding Protein 2) (SMOC-2) (Smooth Muscle-Associated Protein 2) Z44808 Transcripts which are Detectable by Amplicon as Depicted in Sequence Name Z44808junc8-11 (SEQ ID NO: 1291) in Normal and Cancerous Colon Tissues

Expression of SMO2_HUMAN SPARC related modular calcium-binding protein 2 precursor (Secreted modular calcium-binding protein 2) (SMOC-2) (Smooth muscle-associated protein 2) transcripts detectable by or according to junc8-11, Z44808junc8-11 amplicon (SEQ ID NO: 1291) and primers Z44808junc8-11 (SEQ ID NO: 1289) and Z44808junc8-11R (SEQ ID NO: 1290) was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon —HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

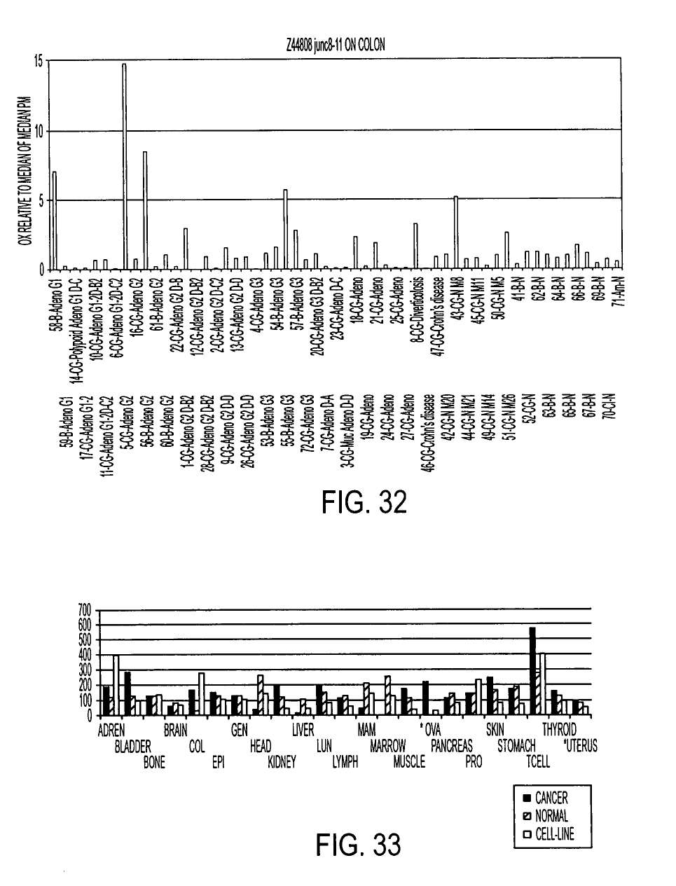

FIG. 32 is a histogram showing over expression of the above-indicated SMO2_HUMAN SPARC related modular calcium-binding protein 2 precursor (Secreted modular calcium-binding protein 2) (SMOC-2) (Smooth muscle-associated protein 2) transcripts in cancerous colon samples relative to the normal samples.

As is evident from FIG. 32, the expression of SMO2_HUMAN SPARC related modular calcium-binding protein 2 precursor (Secreted modular calcium-binding protein 2) (SMOC-2) (Smooth muscle-associated protein 2) transcripts detectable by the above amplicon in cancer samples was higher in a few samples than in the non-cancerous samples (Sample Nos. 41, 52, 62-67, 69-71, Table 1, above, “Tissue samples in testing panel”). Notably an over-expression of at least 5 fold was found in 4 out of 36 adenocarcinoma samples.

Primer pairs are also optionally and preferably encompassed within the present invention; for example, for the above experiment, the following primer pair was used as a non-limiting illustrative example only of a suitable primer pair: Z44808junc8-11F forward primer (SEQ ID NO: 1289); and Z44808junc8-11R reverse primer (SEQ ID NO: 1290).

The present invention also preferably encompasses any amplicon obtained through the use of any suitable primer pair; for example, for the above experiment, the following amplicon was obtained as a non-limiting illustrative example only of a suitable amplicon: Z44808junc8-11 (SEQ ID NO: 1291).

Primers:
Forward primer Z44808junc8-11F (SEQ ID NO: 1289):
GAAGGCACAGGAAAAACAGATATTG
Reverse primer Z44808junc8-11R (SEQ ID NO: 1290):
TGGTGCTCTTGGTCACAGGAT
Amplicon Z44808junc8-11 (SEQ ID NO: 1291):
GAAGGCACAGGAAAAACAGATATTGCATCACGTTACCCTACCCTTTGGAC
TGAACAGGTTAAAAGTCGGCAGAACAAAACCAATAAGAATTCAGTGTCAT
CCTGTGACCAAGAGCACCA

### Expression of SMO2_HUMAN SPARC Related Modular Calcium-Binding Protein 2 Precursor (Secreted Modular Calcium-Binding Protein 2) (SMOC-2) (Smooth Muscle-Associated Protein 2) Z44808 Transcripts which are Detectable by Amplicon as Depicted in Sequence Name Z44808 junc8-11 (SEQ ID NO: 1291) in Different Normal Tissues

Expression of SMO2_HUMAN SPARC related modular calcium-binding protein 2 precursor (Secreted modular calcium-binding protein 2) (SMOC-2) (Smooth muscle-associated protein 2) transcripts detectable by or according to Z44808 junc8-11 amplicon (SEQ ID NO: 1291) and primers: Z44808 junc8-11F (SEQ ID NO: 1289) and Z44808 junc8-11R (SEQ ID NO: 1290) was measured by real time PCR. In parallel the expression of four housekeeping genes —RPL19 (GenBank Accession No. NM_{—}000981 (SEQ ID NO:1580); RPL19 amplicon, SEQ ID NO:1264), TATA box (GenBank Accession No. NM_{—}003194 (SEQ ID NO:1581); TATA amplicon, SEQ ID NO:1267), Ubiquitin (GenBank Accession No. BC000449 (SEQ ID NO:1582); amplicon-Ubiquitin-amplicon) and SDHA (GenBank Accession No. NM_{—}004168 (SEQ ID NO:1583); amplicon—SDHA-amplicon, SEQ ID NO:1273) was measured similarly. For each RT sample, the expression of the above amplicon was normalized to the geometric mean of the quantities of the housekeeping genes. The normalized quantity of each RT sample was then divided by the median of the quantities of the ovary samples (Sample Nos. 18-20, Table 2, “Tissue samples in normal panel”), to obtain a value of relative expression of each sample relative to median of the ovary samples.

Primers:
Forward primer Z44808junc8-11F (SEQ ID NO: 1289):
GAAGGCACAGGAAAAACAGATATTG
Reverse primer Z44808junc8-11R (SEQ ID NO: 1290):
TGGTGCTCTTGGTCACAGGAT
Amplicon Z44808junc8-11 (SEQ ID NO: 1291):
GAAGGCACAGGAAAAACAGATATTGCATCACGTTACCCTACCCTTTGGAC
TGAACAGGTTAAAAGTCGGCAGAACAAAACCAATAAGAATTCAGTGTCAT
CCTGTGACCAAGAGCACCA

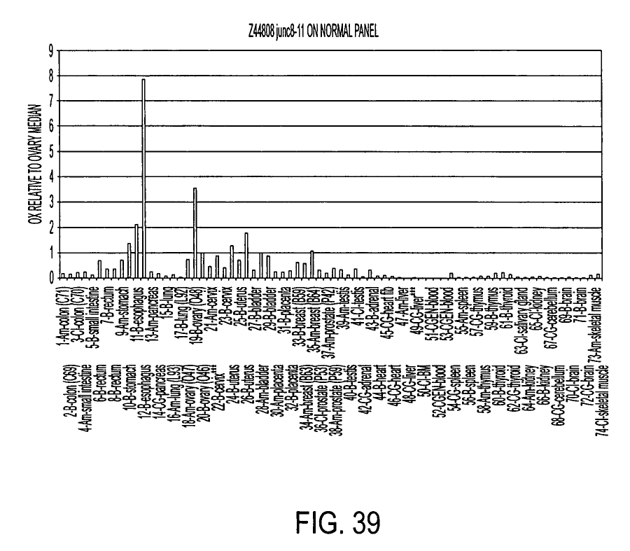

The results are shown in FIG. 39, demonstrating the expression of SMO2_HUMAN SPARC related modular calcium-binding protein 2 precursor (Secreted modular calcium-binding protein 2) (SMOC-2) (Smooth muscle-associated protein 2) Z44808 transcripts which are detectable by amplicon as depicted in sequence name Z44808 junc8-11 (SEQ ID NO: 1291) in different normal tissues.

### Description for Cluster Z25299

Cluster Z25299 features 5 transcript(s) and 11 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| Z25299_PEA_2_T1 | 28 |
| Z25299_PEA_2_T2 | 29 |
| Z25299_PEA_2_T3 | 30 |
| Z25299_PEA_2_T6 | 31 |
| Z25299_PEA_2_T9 | 32 |

TABLE 2
Segments of interest

| Segment Name | SEQ ID NO: |
|---|---|
| Z25299_PEA_2_node_20 | 241 |
| Z25299_PEA_2_node_21 | 242 |
| Z25299_PEA_2_node_23 | 243 |
| Z25299_PEA_2_node_24 | 244 |
| Z25299_PEA_2_node_8 | 245 |
| Z25299_PEA_2_node_12 | 246 |
| Z25299_PEA_2_node_13 | 247 |
| Z25299_PEA_2_node_14 | 248 |
| Z25299_PEA_2_node_17 | 249 |
| Z25299_PEA_2_node_18 | 250 |
| Z25299_PEA_2_node_19 | 251 |

TABLE 3
Proteins of interest

| Protein Name | SEQ ID NO: |
|---|---|
| Z25299_PEA_2_P2 | 560 |
| Z25299_PEA_2_P3 | 561 |
| Z25299_PEA_2_P7 | 562 |
| Z25299_PEA_2_P10 | 563 |

These sequences are variants of the known protein Antileukoproteinase 1 precursor (SwissProt accession identifier ALK1_HUMAN; known also according to the synonyms ALP; HUSI-1; Seminal proteinase inhibitor; Secretory leukocyte protease inhibitor; BLPI; Mucus proteinase inhibitor; MPI; WAP four-disulfide core domain protein 4; Protease inhibitor WAP4), SEQ ID NO: 625, referred to herein as the previously known protein.

Protein Antileukoproteinase 1 precursor (SEQ ID NO:625) is known or believed to have the following function(s): Acid-stable proteinase inhibitor with strong affinities for trypsin, chymotrypsin, elastase, and cathepsin G. May prevent elastase-mediated damage to oral and possibly other mucosal tissues. The sequence for protein Antileukoproteinase 1 precursor is given at the end of the application, as “Antileukoproteinase 1 precursor amino acid sequence”. Protein Antileukoproteinase 1 precursor localization is believed to be Secreted.

It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Elastase inhibitor; Tryptase inhibitor. A therapeutic role for a protein represented by the cluster has been predicted. The cluster was assigned this field because there was information in the drug database or the public databases (e.g., described herein above) that this protein, or part thereof, is used or can be used for a potential therapeutic indication: Anti-inflammatory; Antiasthma.

The following GO Annotation(s) apply to the previously known protein. The following annotation(s) were found: proteinase inhibitor; serine protease inhibitor, which are annotation(s) related to Molecular Function.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster Z25299 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

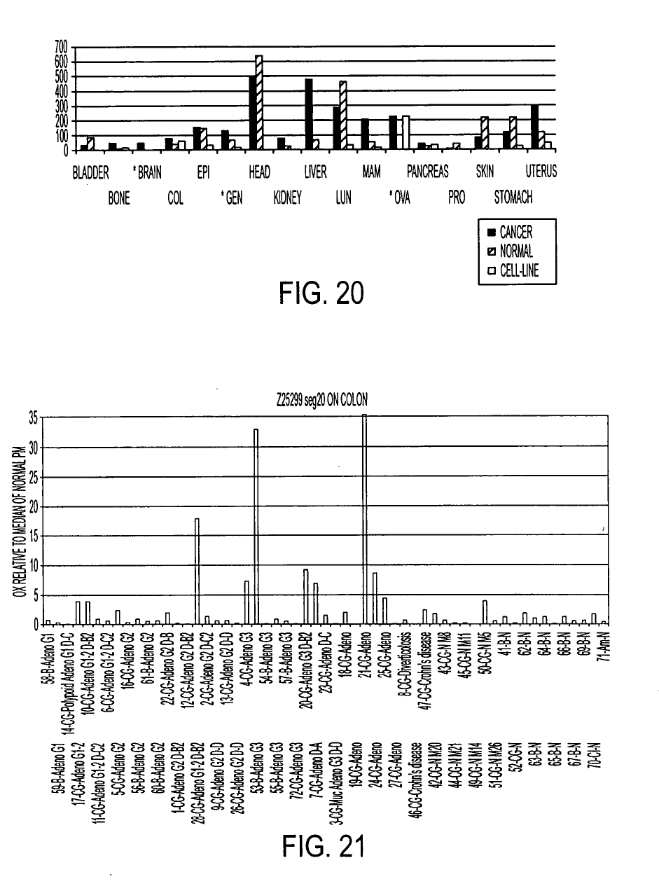

Overall, the following results were obtained as shown with regard to the histograms in FIG. 20 and Table 4. This cluster is overexpressed (at least at a minimum level) in the following pathological conditions: brain malignant tumors, a mixture of malignant tumors from different tissues and ovarian carcinoma.

TABLE 4
Normal tissue distribution

| Name of Tissue | Number |
|---|---|
| Bladder | 82 |
| Bone | 6 |
| Brain | 0 |
| colon | 37 |
| epithelial | 145 |
| general | 73 |
| head and neck | 638 |
| kidney | 26 |
| liver | 68 |
| lung | 465 |
| breast | 52 |
| ovary | 0 |
| pancreas | 20 |
| prostate | 36 |
| skin | 215 |
| stomach | 219 |
| uterus | 113 |

TABLE 5
P values and ratios for expression in cancerous tissue

| Name of Tissue | P1 | P2 | SP1 | R3 | SP2 | R4 |
|---|---|---|---|---|---|---|
| bladder | 8.2e−01 | 8.5e−01 | 9.2e−01 | 0.6 | 9.7e−01 | 0.5 |
| bone | 5.5e−01 | 7.3e−01 | 4.0e−01 | 2.1 | 4.9e−01 | 1.5 |
| brain | 8.8e−02 | 1.5e−01 | 2.3e−03 | 7.7 | 1.2e−02 | 4.8 |
| colon | 3.3e−01 | 2.8e−01 | 4.2e−01 | 1.6 | 4.2e−01 | 1.5 |
| epithelial | 2.5e−01 | 7.6e−01 | 3.8e−01 | 1.0 | 1 | 0.6 |
| general | 6.4e−03 | 2.5e−01 | 1.7e−06 | 1.6 | 5.2e−01 | 0.9 |
| head and neck | 3.6e−01 | 5.9e−01 | 7.6e−01 | 0.6 | 1 | 0.3 |
| kidney | 7.4e−01 | 8.4e−01 | 2.1e−01 | 2.1 | 4.2e−01 | 1.4 |
| liver | 4.1e−01 | 9.1e−01 | 4.2e−02 | 3.2 | 6.4e−01 | 0.8 |
| lung | 7.6e−01 | 8.3e−01 | 9.8e−01 | 0.5 | 1 | 0.3 |
| breast | 5.0e−01 | 5.5e−01 | 9.8e−02 | 1.6 | 3.4e−01 | 1.1 |
| ovary | 3.7e−02 | 3.0e−02 | 6.9e−03 | 6.1 | 4.9e−03 | 5.6 |
| pancreas | 3.8e−01 | 3.6e−01 | 3.6e−01 | 1.7 | 3.9e−01 | 1.5 |
| prostate | 9.1e−01 | 9.2e−01 | 8.9e−01 | 0.5 | 9.4e−01 | 0.5 |
| skin | 6.0e−01 | 8.1e−01 | 9.3e−01 | 0.4 | 1 | 0.1 |
| stomach | 3.0e−01 | 8.1e−01 | 9.1e−01 | 0.6 | 1 | 0.3 |
| uterus | 1.6e−01 | 1.3e−01 | 3.2e−02 | 1.6 | 3.0e−01 | 1.1 |

As noted above, cluster Z25299 features 5 transcript(s), which were listed in Table 1 above. These transcript(s) encode for protein(s) which are variant(s) of protein Antileukoproteinase 1 precursor (SEQ ID NO:625). A description of each variant protein according to the present invention is now provided.

Variant protein Z25299_PEA_{—}2_P2 (SEQ ID NO:560) according to the present invention has an amino acid sequence as given at the end of the application; it is encoded by transcript(s) Z25299_PEA_{—}2_T1 (SEQ ID NO:28). An alignment is given to the known protein (Antileukoproteinase 1 precursor (SEQ ID NO:625)) at the end of the application. One or more alignments to one or more previously published protein sequences are given at the end of the application. A brief description of the relationship of the variant protein according to the present invention to each such aligned protein is as follows:

Comparison Report Between Z25299_PEA_{—}2_P2 (SEQ ID NO:560) and ALK1_HUMAN (SEQ ID NO:625):

1. An isolated chimeric polypeptide encoding for Z25299_PEA_{—}2_P2 (SEQ ID NO:560), comprising a first amino acid sequence being at least 90% homologous to MKSSGLFPFLVLLALGTLAPWAVEGSGKSFKAGVCPPKKSAQCLRYKKPECQSDWQCPGKKRCCPDTCGI KCLDPVDTPNPTRRKPGKCPVTYGQCLMLNPPNFCEMDGQCKRDLKCCMGMCGKSCVSPVK corresponding to amino acids 1-131 of ALK1_HUMAN (SEQ ID NO:625), which also corresponds to amino acids 1-131 of Z25299_PEA_{—}2_P2 (SEQ ID NO:560), and a second amino acid sequence being at least 70%, optionally at least 80%, preferably at least 85%, more preferably at least 90% and most preferably at least 95% homologous to a polypeptide having the sequence GKQGMRAH (SEQ ID NO:1503) corresponding to amino acids 132-139 of Z25299_PEA_{—}2_P2 (SEQ ID NO:560), wherein said first and second amino acid sequences are contiguous and in a sequential order.

2. An isolated polypeptide encoding for a tail of Z25299_PEA_{—}2_P2 (SEQ ID NO:560), comprising a polypeptide being at least 70%, optionally at least about 80%, preferably at least about 85%, more preferably at least about 90% and most preferably at least about 95% homologous to the sequence GKQGMRAH (SEQ ID NO:1503) in Z25299_PEA_{—}2_P2 (SEQ ID NO:560).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein Z25299_PEA_{—}2_P2 (SEQ ID NO:560) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein Z25299_PEA_{—}2_P2 (SEQ ID NO:560) sequence provides support for the deduced sequence of this variant protein according to the present invention).

TABLE 6
Amino acid mutations

| SNP position(s) | | |
|---|---|---|
| on amino acid | Alternative | Previously |
| sequence | amino acid(s) | known SNP? |
| 136 | M -> T | Yes |
| 20 | P -> | No |
| 43 | C -> R | No |
| 48 | K -> N | No |
| 83 | R -> K | No |
| 84 | R -> W | No |

Variant protein Z25299_PEA_{—}2_P2 (SEQ ID NO:560) is encoded by the following transcript(s): Z25299_PEA_{—}2_T1 (SEQ ID NO:28), for which the sequence(s) is/are given at the end of the application. The coding portion of transcript Z25299_PEA_{—}2_T1 (SEQ ID NO:28) is shown in bold; this coding portion starts at position 124 and ends at position 540. The transcript also has the following SNPs as listed in Table 7 (given according to their position on the nucleotide sequence, with the alternative nucleic acid listed; the last column indicates whether the SNP is known or not; the presence of known SNPs in variant protein Z25299_PEA_{—}2_P2 (SEQ ID NO:560) sequence provides support for the deduced sequence of this variant protein according to the present invention).

TABLE 7
Nucleic acid SNPs

| SNP position on | Alternative | Previously |
|---|---|---|
| nucleotide sequence | nucleic acid | known SNP? |
| 122 | C -> T | No |
| 123 | C -> T | No |
| 530 | T -> C | Yes |
| 989 | C -> T | Yes |
| 1127 | C -> T | Yes |
| 1162 | A -> C | Yes |
| 1180 | A -> C | Yes |
| 1183 | A -> C | Yes |
| 1216 | A -> C | Yes |
| 1262 | G -> A | Yes |
| 183 | T -> | No |
| 250 | T -> C | No |
| 267 | A -> C | No |
| 267 | A -> G | No |
| 339 | C -> T | Yes |
| 371 | G -> A | No |
| 373 | A -> T | No |
| 435 | C -> T | No |

Variant protein Z25299_PEA_{—}2_P3 (SEQ ID NO:561) according to the present invention has an amino acid sequence as given at the end of the application; it is encoded by transcript(s) Z25299_PEA_{—}2_T2 (SEQ ID NO:29). An alignment is given to the known protein (Antileukoproteinase 1 precursor (SEQ ID NO:625)) at the end of the application. One or more alignments to one or more previously published protein sequences are given at the end of the application. A brief description of the relationship of the variant protein according to the present invention to each such aligned protein is as follows:

Comparison Report Between Z25299_PEA_{—}2_P3 (SEQ ID NO:561) and ALK1_HUMAN (SEQ ID NO:625):

1. An isolated chimeric polypeptide encoding for Z25299_PEA_{—}2_P3 (SEQ ID NO:561), comprising a first amino acid sequence being at least 90% homologous to MKSSGLFPFLVLLALGTLAPWAVEGSGKSFKAGVCPPKKSAQCLRYKKPECQSDWQCPGKKRCCPDTCGI KCLDPVDTPNPTRRKPGKCPVTYGQCLMLNPPNFCEMDGQCKRDLKCCMGMCGKSCVSPVK corresponding to amino acids 1-131 of ALK1_HUMAN (SEQ ID NO:625), which also corresponds to amino acids 1-131 of Z25299_PEA_{—}2_P3 (SEQ ID NO:561), and a second amino acid sequence being at least 70%, optionally at least 80%, preferably at least 85%, more preferably at least 90% and most preferably at least 95% homologous to a polypeptide having the sequence GEKRHHKQLRDQEVDPLEMRRHSAG (SEQ ID NO:1504) corresponding to amino acids 132-156 of Z25299_PEA_{—}2_P3 (SEQ ID NO:561), wherein said first and second amino acid sequences are contiguous and in a sequential order.

2. An isolated polypeptide encoding for a tail of Z25299_PEA_{—}2_P3 (SEQ ID NO:561), comprising a polypeptide being at least 70%, optionally at least about 80%, preferably at least about 85%, more preferably at least about 90% and most preferably at least about 95% homologous to the sequence GEKRHHKQLRDQEVDPLEMRRHSAG (SEQ ID NO:1504) in Z25299_PEA_{—}2_P3 (SEQ ID NO:561).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein Z25299_PEA_{—}2_P3 (SEQ ID NO:561) also has the following non-silent SNPs (Single Nucleotide Polymorphisms) as listed in Table 8, (given according to their position(s) on the amino acid sequence, with the alternative amino acid(s) listed; the last column indicates whether the SNP is known or not; the presence of known SNPs in variant protein Z25299_PEA_{—}2_P3 (SEQ ID NO:561) sequence provides support for the deduced sequence of this variant protein according to the present invention).

TABLE 8
Amino acid mutations

| SNP position(s) on amino acid | Alternative | |
|---|---|---|
| sequence | amino acid(s) | Previously known SNP? |
| 20 | P -> | No |
| 43 | C -> R | No |
| 48 | K -> N | No |
| 83 | R -> K | No |
| 84 | R -> W | No |

Variant protein Z25299_PEA_{—}2_P3 (SEQ ID NO:561) is encoded by the following transcript(s): Z25299_PEA_{—}2_T2 (SEQ ID NO:29), for which the sequence(s) is/are given at the end of the application. The coding portion of transcript Z25299_PEA_{—}2_T2 (SEQ ID NO:29) is shown in bold; this coding portion starts at position 124 and ends at position 591. The transcript also has the following SNPs as listed in Table 9 (given according to their position on the nucleotide sequence, with the alternative nucleic acid listed; the last column indicates whether the SNP is known or not; the presence of known SNPs in variant protein Z25299_PEA_{—}2_P3 (SEQ ID NO:561) sequence provides support for the deduced sequence of this variant protein according to the present invention).

TABLE 9
Nucleic acid SNPs

| SNP position on | Alternative | Previously |
|---|---|---|
| nucleotide sequence | nucleic acid | known SNP? |
| 122 | C -> T | No |
| 123 | C -> T | No |
| 183 | T -> | No |
| 250 | T -> C | No |
| 267 | A -> C | No |
| 267 | A -> G | No |
| 339 | C -> T | Yes |
| 371 | G -> A | No |
| 373 | A -> T | No |
| 435 | C -> T | No |

Variant protein Z25299_PEA_{—}2_P7 (SEQ ID NO:562) according to the present invention has an amino acid sequence as given at the end of the application; it is encoded by transcript(s) Z25299_PEA_{—}2_T6 (SEQ ID NO:31). An alignment is given to the known protein (Antileukoproteinase 1 precursor (SEQ ID NO:625)) at the end of the application. One or more alignments to one or more previously published protein sequences are given at the end of the application. A brief description of the relationship of the variant protein according to the present invention to each such aligned protein is as follows:

Comparison Report Between Z25299_PEA_{—}2_P7 (SEQ ID NO:562) and ALK1_HUMAN (SEQ ID NO:625):

1. An isolated chimeric polypeptide encoding for Z25299_PEA_{—}2_P7 (SEQ ID NO:562), comprising a first amino acid sequence being at least 90% homologous to MKSSGLFPFLVLLALGTLAPWAVEGSGKSFKAGVCPPKKSAQCLRYKKPECQSDWQCPGKKRCCPDTCGI KCLDPVDTPNP corresponding to amino acids 1-81 of ALK1_HUMAN (SEQ ID NO:625), which also corresponds to amino acids 1-81 of Z25299_PEA_{—}2_P7 (SEQ ID NO:562), and a second amino acid sequence being at least 70%, optionally at least 80%, preferably at least 85%, more preferably at least 90% and most preferably at least 95% homologous to a polypeptide having the sequence RGSLGSAQ (SEQ ID NO:1505) corresponding to amino acids 82-89 of Z25299_PEA_{—}2_P7 (SEQ ID NO:562), wherein said first and second amino acid sequences are contiguous and in a sequential order.

2. An isolated polypeptide encoding for a tail of Z25299_PEA_{—}2_P7 (SEQ ID NO:562), comprising a polypeptide being at least 70%, optionally at least about 80%, preferably at least about 85%, more preferably at least about 90% and most preferably at least about 95% homologous to the sequence RGSLGSAQ (SEQ ID NO:1505) in Z25299_PEA_{—}2_P7 (SEQ ID NO:562).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein Z25299_PEA_{—}2_P7 (SEQ ID NO:562) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein Z25299_PEA_{—}2_P7 (SEQ ID NO:562) sequence provides support for the deduced sequence of this variant protein according to the present invention).

TABLE 10
Amino acid mutations

| SNP position(s) on amino acid | Alternative | |
|---|---|---|
| sequence | amino acid(s) | Previously known SNP? |
| 20 | P -> | No |
| 43 | C -> R | No |
| 48 | K -> N | No |
| 82 | R -> S | No |

Variant protein Z25299_PEA_{—}2_P7 (SEQ ID NO:562) is encoded by the following transcript(s): Z25299_PEA_{—}2_T6 (SEQ ID NO:31), for which the sequence(s) is/are given at the end of the application. The coding portion of transcript Z25299_PEA_{—}2_T6 (SEQ ID NO:31) is shown in bold; this coding portion starts at position 124 and ends at position 390. The transcript also has the following SNPs as listed in Table 11 (given according to their position on the nucleotide sequence, with the alternative nucleic acid listed; the last column indicates whether the SNP is known or not; the presence of known SNPs in variant protein Z25299_PEA_{—}2_P7 (SEQ ID NO:562) sequence provides support for the deduced sequence of this variant protein according to the present invention).

TABLE 11
Nucleic acid SNPs

| SNP position on | | |
|---|---|---|
| nucleotide sequence | Alternative nucleic acid | Previously known SNP? |
| 122 | C -> T | No |
| 123 | C -> T | No |
| 576 | A -> C | Yes |
| 594 | A -> C | Yes |
| 597 | A -> C | Yes |
| 630 | A -> C | Yes |
| 676 | G -> A | Yes |
| 183 | T -> | No |
| 250 | T -> C | No |
| 267 | A -> C | No |
| 267 | A -> G | No |
| 339 | C -> T | Yes |
| 369 | A -> T | No |
| 431 | C -> T | No |
| 541 | C -> T | Yes |

Variant protein Z25299_PEA_{—}2_P10 (SEQ ID NO:563) according to the present invention has an amino acid sequence as given at the end of the application; it is encoded by transcript(s) Z25299_PEA_{—}2_T9 (SEQ ID NO:32). An alignment is given to the known protein (Antileukoproteinase 1 precursor (SEQ ID NO:625)) at the end of the application. One or more alignments to one or more previously published protein sequences are given at the end of the application. A brief description of the relationship of the variant protein according to the present invention to each such aligned protein is as follows:

Comparison Report Between Z25299_PEA_{—}2_P10 (SEQ ID NO:563) and ALK1_HUMAN (SEQ ID NO:625):

1. An isolated chimeric polypeptide encoding for Z25299_PEA_{—}2_P10 (SEQ ID NO:563), comprising a first amino acid sequence being at least 90% homologous to MKSSGLFPFLVLLALGTLAPWAVEGSGKSFKAGVCPPKKSAQCLRYKKPECQSDWQCPGKKRCCPDTCGI KCLDPVDTPNPT corresponding to amino acids 1-82 of ALK1_HUMAN (SEQ ID NO:625), which also corresponds to amino acids 1-82 of Z25299_PEA_{—}2_P10 (SEQ ID NO:563).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein Z25299_PEA_{—}2_P10 (SEQ ID NO:563) also has the following non-silent SNPs (Single Nucleotide Polymorphisms) as listed in Table 12, (given according to their position(s) on the amino acid sequence, with the alternative amino acid(s) listed; the last column indicates whether the SNP is known or not; the presence of known SNPs in variant protein Z25299_PEA_{—}2_P10 (SEQ ID NO:563) sequence provides support for the deduced sequence of this variant protein according to the present invention).

TABLE 12
Amino acid mutations

| SNP position(s) on amino acid | Alternative | |
|---|---|---|
| sequence | amino acid(s) | Previously known SNP? |
| 20 | P -> | No |
| 43 | C -> R | No |
| 48 | K -> N | No |

Variant protein Z25299_PEA_{—}2_P10 (SEQ ID NO:563) is encoded by the following transcript(s): Z25299_PEA_{—}2_T9 (SEQ ID NO:32), for which the sequence(s) is/are given at the end of the application. The coding portion of transcript Z25299_PEA_{—}2_T9 (SEQ ID NO:32) is shown in bold; this coding portion starts at position 124 and ends at position 369. The transcript also has the following SNPs as listed in Table 13 (given according to their position on the nucleotide sequence, with the alternative nucleic acid listed; the last column indicates whether the SNP is known or not; the presence of known SNPs in variant protein Z25299_PEA_{—}2_P10 (SEQ ID NO:563) sequence provides support for the deduced sequence of this variant protein according to the present invention).

TABLE 13
Nucleic acid SNPs

| SNP position on | | |
|---|---|---|
| nucleotide sequence | Alternative nucleic acid | Previously known SNP? |
| 122 | C -> T | No |
| 123 | C -> T | No |
| 451 | A -> C | Yes |
| 484 | A -> C | Yes |
| 530 | G -> A | Yes |
| 183 | T -> | No |
| 250 | T -> C | No |
| 267 | A -> C | No |
| 267 | A -> G | No |
| 339 | C -> T | Yes |
| 395 | C -> T | Yes |
| 430 | A -> C | Yes |
| 448 | A -> C | Yes |

As noted above, cluster Z25299 features 11 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster Z25299_PEA_{—}2_node_{—}20 (SEQ ID NO:241) according to the present invention is supported by 6 libraries. The number of libraries was determined as previously described. This segment can be found in the following transcript(s): Z25299_PEA_{—}2_T1 (SEQ ID NO:28). Table 14 below describes the starting and ending position of this segment on each transcript.

TABLE 14
Segment location on transcripts

| | Segment | |
|---|---|---|
| | starting | Segment ending |
| Transcript name | position | position |
| Z25299_PEA_2_T1 (SEQ ID NO: 28) | 518 | 1099 |

Segment cluster Z25299_PEA_{—}2_node_{—}21 (SEQ ID NO:242) according to the present invention is supported by 162 libraries. The number of libraries was determined as previously described. This segment can be found in the following transcript(s): Z25299_PEA_{—}2_T1 (SEQ ID NO:28), Z25299_PEA_{—}2_T6 (SEQ ID NO:31) and Z25299_PEA_{—}2_T9 (SEQ ID NO:32). Table 15 below describes the starting and ending position of this segment on each transcript.

TABLE 15
Segment location on transcripts

| | Segment | |
|---|---|---|
| | starting | Segment ending |
| Transcript name | position | position |
| Z25299_PEA_2_T1 (SEQ ID NO: 28) | 1100 | 1292 |
| Z25299_PEA_2_T6 (SEQ ID NO: 31) | 514 | 706 |
| Z25299_PEA_2_T9 (SEQ ID NO: 32) | 368 | 560 |

Segment cluster Z25299_PEA_{—}2_node_{—}23 (SEQ ID NO:243) according to the present invention is invention is supported by 2 libraries. The number of libraries was determined as previously described. This segment can be found in the following transcript(s): Z25299_PEA_{—}2_T2 (SEQ ID NO:29). Table 16 below describes the starting and ending position of this segment on each transcript.

TABLE 16
Segment location on transcripts

| | Segment | |
|---|---|---|
| | starting | Segment |
| Transcript name | position | ending position |
| Z25299_PEA_2_T2 (SEQ ID NO: 29) | 518 | 707 |

Segment cluster Z25299_PEA_{—}2_node_{—}24 (SEQ ID NO:244) according to the present invention is supported by 2 libraries. The number of libraries was determined as previously described. This segment can be found in the following transcript(s): Z25299_PEA_{—}2_T2 (SEQ ID NO:29) and Z25299_PEA_{—}2_T3 (SEQ ID NO:30). Table 17 below describes the starting and ending position of this segment on each transcript.

TABLE 17
Segment location on transcripts

| | Segment | |
|---|---|---|
| | starting | Segment ending |
| Transcript name | position | position |
| Z25299_PEA_2_T2 (SEQ ID NO: 29) | 708 | 886 |
| Z25299_PEA_2_T3 (SEQ ID NO: 30) | 518 | 696 |

Segment cluster Z25299_PEA_{—}2_node_{—}8 (SEQ ID NO:245) according to the present invention is supported by 218 libraries. The number of libraries was determined as previously described. This segment can be found in the following transcript(s): Z25299_PEA_{—}2_T1 (SEQ ID NO:28), Z25299_PEA_{—}2_T2 (SEQ ID NO:29), Z25299_PEA_{—}2_T3 (SEQ ID NO:30), Z25299_PEA_{—}2_T6 (SEQ ID NO:31) and Z25299_PEA_{—}2_T9 (SEQ ID NO:32). Table 18 below describes the starting and ending position of this segment on each transcript.

TABLE 18
Segment location on transcripts

| | Segment | |
|---|---|---|
| | starting | Segment ending |
| Transcript name | position | position |
| Z25299_PEA_2_T1 (SEQ ID NO: 28) | 1 | 208 |
| Z25299_PEA_2_T2 (SEQ ID NO: 29) | 1 | 208 |
| Z25299_PEA_2_T3 (SEQ ID NO: 30) | 1 | 208 |
| Z25299_PEA_2_T6 (SEQ ID NO: 31) | 1 | 208 |
| Z25299_PEA_2_T9 (SEQ ID NO: 32) | 1 | 208 |

According to an optional embodiment of the present invention, short segments related to the above cluster are also provided. These segments are up to about 120 bp in length, and so are included in a separate description.

Segment cluster Z25299_PEA_{—}2_node_{—}12 (SEQ ID NO:246) according to the present invention is supported by 228 libraries. The number of libraries was determined as previously described. This segment can be found in the following transcript(s): Z25299_PEA_{—}2_T1 (SEQ ID NO:28), Z25299_PEA_{—}2_T2 (SEQ ID NO:29), Z25299_PEA_{—}2_T3 (SEQ ID NO:30), Z25299_PEA_{—}2_T6 (SEQ ID NO:31) and Z25299_PEA_{—}2_T9 (SEQ ID NO:32). Table 20 below describes the starting and ending position of this segment on each transcript.

TABLE 20
Segment location on transcripts

| | Segment | |
|---|---|---|
| | starting | Segment ending |
| Transcript name | position | position |
| Z25299_PEA_2_T1 (SEQ ID NO: 28) | 209 | 245 |
| Z25299_PEA_2_T2 (SEQ ID NO: 29) | 209 | 245 |
| Z25299_PEA_2_T3 (SEQ ID NO: 30) | 209 | 245 |
| Z25299_PEA_2_T6 (SEQ ID NO: 31) | 209 | 245 |
| Z25299_PEA_2_T9 (SEQ ID NO: 32) | 209 | 245 |

Segment cluster Z25299_PEA_{—}2_node_{—}13 (SEQ ID NO:247) according to the present invention is supported by 246 libraries. The number of libraries was determined as previously described. This segment can be found in the following transcript(s): Z25299_PEA_{—}2_T1 (SEQ ID NO:28), Z25299_PEA_{—}2_T2 (SEQ ID NO:29), Z25299_PEA_{—}2_T3 (SEQ ID NO:30) Z25299_PEA_{—}2_T6 (SEQ ID NO:31) and Z25299_PEA_{—}2_T9 (SEQ ID NO:32). Table 22 below describes the starting and ending position of this segment on each transcript.

TABLE 22
Segment location on transcripts

| | Segment | |
|---|---|---|
| | starting | Segment ending |
| Transcript name | position | position |
| Z25299_PEA_2_T1 (SEQ ID NO: 28) | 246 | 357 |
| Z25299_PEA_2_T2 (SEQ ID NO: 29) | 246 | 357 |
| Z25299_PEA_2_T3 (SEQ ID NO: 30) | 246 | 357 |
| Z25299_PEA_2_T6 (SEQ ID NO: 31) | 246 | 357 |
| Z25299_PEA_2_T9 (SEQ ID NO: 32) | 246 | 357 |

Segment cluster Z25299_PEA_{—}2_node_{—}14 (SEQ ID NO:248) according to the present invention can be found in the following transcript(s): Z25299_PEA_{—}2_T1 (SEQ ID NO:28), Z25299_PEA_{—}2_T2 (SEQ ID NO:29), Z25299_PEA_{—}2_T3 (SEQ ID NO:30), Z25299_PEA_{—}2_T6 (SEQ ID NO:31) and Z25299_PEA_{—}2_T9 (SEQ ID NO:32). Table 23 below describes the starting and ending position of this segment on each transcript.

TABLE 23
Segment location on transcripts

| | Segment | |
|---|---|---|
| | starting | Segment ending |
| Transcript name | position | position |
| Z25299_PEA_2_T1 (SEQ ID NO: 28) | 358 | 367 |
| Z25299_PEA_2_T2 (SEQ ID NO: 29) | 358 | 367 |
| Z25299_PEA_2_T3 (SEQ ID NO: 30) | 358 | 367 |
| Z25299_PEA_2_T6 (SEQ ID NO: 31) | 358 | 367 |
| Z25299_PEA_2_T9 (SEQ ID NO: 32) | 358 | 367 |

Segment cluster Z25299_PEA_{—}2_node_{—}17 (SEQ ID NO:249) according to the present invention can be found in the following transcript(s): Z25299_PEA_{—}2_T1 (SEQ ID NO:28), Z25299_PEA_{—}2_T2 (SEQ ID NO:29) and Z25299_PEA_{—}2_T3 (SEQ ID NO:30). Table 24 below describes the starting and ending position of this segment on each transcript.

TABLE 24
Segment location on transcripts

| | Segment | |
|---|---|---|
| | starting | Segment ending |
| Transcript name | position | position |
| Z25299_PEA_2_T1 (SEQ ID NO: 28) | 368 | 371 |
| Z25299_PEA_2_T2 (SEQ ID NO: 29) | 368 | 371 |
| Z25299_PEA_2_T3 (SEQ ID NO: 30) | 368 | 371 |

Segment cluster Z25299_PEA_{—}2_node_{—}18 (SEQ ID NO:250) according to the present invention is supported by 221 libraries. The number of libraries was determined as previously described. This segment can be found in the following transcript(s): Z25299_PEA_{—}2_T1 (SEQ ID NO:28), Z25299_PEA_{—}2_T2 (SEQ ID NO:29), Z25299_PEA_{—}2_T3 (SEQ ID NO:30) and Z25299_PEA_{—}2_T6 (SEQ ID NO:31). Table 25 below describes the starting and ending position of this segment on each transcript.

TABLE 25
Segment location on transcripts

| | Segment | |
|---|---|---|
| | starting | Segment ending |
| Transcript name | position | position |
| Z25299_PEA_2_T1 (SEQ ID NO: 28) | 372 | 427 |
| Z25299_PEA_2_T2 (SEQ ID NO: 29) | 372 | 427 |
| Z25299_PEA_2_T3 (SEQ ID NO: 30) | 372 | 427 |
| Z25299_PEA_2_T6 (SEQ ID NO: 31) | 368 | 423 |

Segment cluster Z25299_PEA_{—}2_node_{—}19 (SEQ ID NO:251) according to the present invention is supported by 197 libraries. The number of libraries was determined as previously described. This segment can be found in the following transcript(s): Z25299_PEA_{—}2_T1 (SEQ ID NO:28), Z25299_PEA_{—}2_T2 (SEQ ID NO:29), Z25299_PEA_{—}2_T3 (SEQ ID NO:30) and Z25299_PEA_{—}2_T6 (SEQ ID NO:31). Table 26 below describes the starting and ending position of this segment on each transcript.

TABLE 26
Segment location on transcripts

| | | Segment |
|---|---|---|
| | Segment starting | ending |
| Transcript name | position | position |
| Z25299_PEA_2_T1 (SEQ ID NO: 28) | 428 | 517 |
| Z25299_PEA_2_T2 (SEQ ID NO: 29) | 428 | 517 |
| Z25299_PEA_2_T3 (SEQ ID NO: 30) | 428 | 517 |
| Z25299_PEA_2_T6 (SEQ ID NO: 31) | 424 | 513 |

### Variant Protein Alignment to the Previously Known Protein:

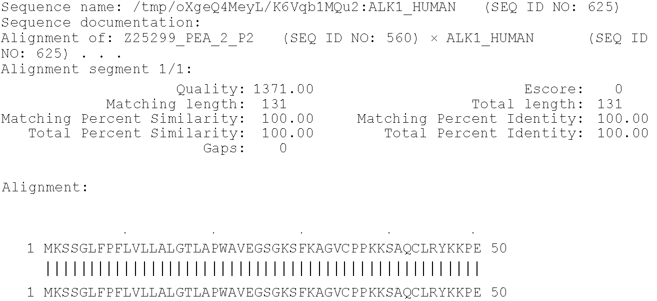

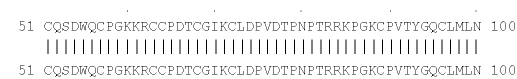

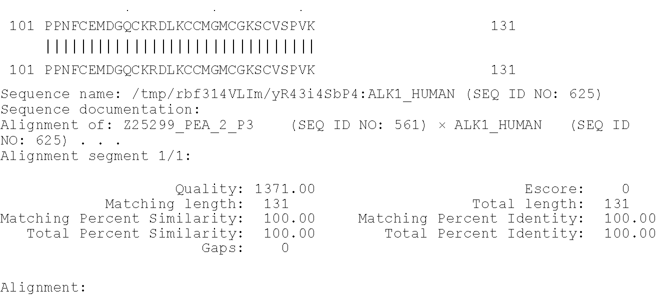

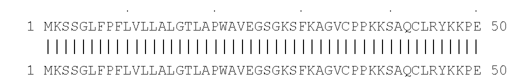

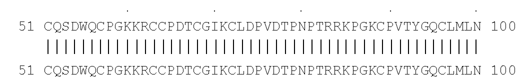

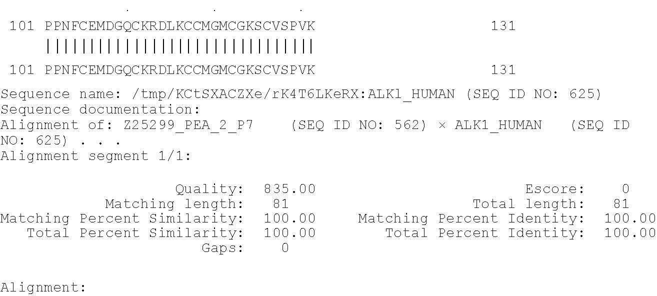

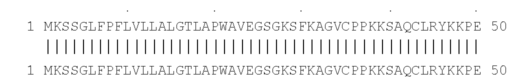

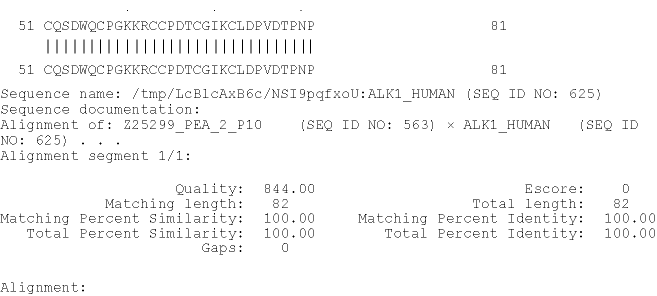

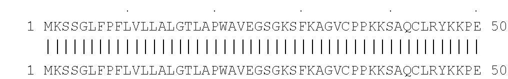

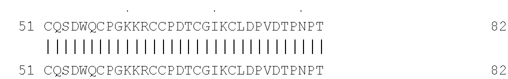

### Expression of Secretory Leukocyte Protease Inhibitor Acid-Stable Proteinase Inhibitor with Strong Affinities for Trypsin, Chymotrypsin, Elastase, and Cathepsin G

### Z25299 Transcripts, which are Detectable by Amplicon as Depicted in Sequence Name Z25299 seg20 (SEQ ID NO:1294), Were Examined for Expression in Normal and Cancerous Colon Tissues

Transcripts detectable by or according to seg20, Z25299 seg20 amplicon (SEQ ID NO: 1294) and Z25299 seg20F (SEQ ID NO: 1292) and Z25299 seg20R (SEQ ID NO: 1293) primers were measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); HPRT1-amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above Tissue samples in testing panel), to obtain a value of fold up-regulation for each sample relative to median of the normal PM samples.

FIG. 21 is a histogram showing over expression of the above-indicated variant.

Transcript expression in cancerous colon samples relative to the normal samples are shown.

As is evident from FIG. 21, transcripts detectable by the above amplicon(s) in cancer samples were significantly higher than in the non-cancerous samples (Sample Nos. 41, 52, 62-67, 69-71 Table 1 Tissue samples in testing panel). Notably an over-expression of at least 5 fold was found in 7 out of 36 adenocarcinoma samples.

Statistical analysis was applied to verify the significance of these results, as described below. The P value for the difference in the expression levels of this variant was determined.

Transcripts detectable by the above amplicon in colon cancer samples versus the normal tissue samples were determined by T test as 6.98E-02.

Threshold of 5 fold overexpression was found to differentiate between cancer and normal samples with P value of 1.33E-02 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: Z25299 seg20F forward primer (SEQ ID NO: 1292); and Z25299 seg20R reverse primer (SEQ ID NO: 1293).

The present invention also preferably encompasses any amplicon obtained through the use of any suitable primer pair; for example, for the above experiment, the following amplicon was obtained as a non-limiting illustrative example only of a suitable amplicon: Z25299 seg20 (SEQ ID NO: 1294).

Forward primer (SEQ ID NO: 1292):
CTCCTGAACCCTACTCCAAGCA
Reverse primer (SEQ ID NO: 1293):
CAGGCGATCCTATGGAAATCC
Amplicon (SEQ ID NO: 1294):
CTCCTGAACCCTACTCCAAGCACAGCCTCTGTCTGACTCCCTTGTCCTTT
CAAGAGAACTGTTCTCCAGGTCTCAGGGCCAGGATTTCCATAGGATCGCC
TG

### Expression of Secretory Leukocyte Protease Inhibitor Acid-Stable Proteinase Inhibitor with Strong Affinities for Trypsin, Chymotrypsin, Elastase, and Cathepsin G. May Prevent Elastase-Mediated Damage to Oral and Possibly Other Mucosal Tissues Z25299 Transcripts which are Detectable by Amplicon as Depicted in Sequence Name Z25299Seg20 (SEQ ID NO: 1294) in Different Normal Tissues

Expression of Secretory leukocyte protease inhibitor Acid-stable proteinase inhibitor with strong affinities for trypsin, chymotrypsin, elastase, and cathepsin G. May prevent elastase-mediated damage to oral and possibly other mucosal tissues transcripts detectable by or according to Z25299seg20 amplicon (SEQ ID NO: 1294) and primers: Z25299seg20F (SEQ ID NO: 1294) and Z25299seg20R (SEQ ID NO: 1294) was measured by real time PCR. In parallel the expression of four housekeeping genes —RPL19 (GenBank Accession No. NM_{—}000981 (SEQ ID NO:1580); RPL19 amplicon), TATA box (GenBank Accession No. NM_{—}003194 (SEQ ID NO:1581); TATA amplicon (SEQ ID NO: 1267)), Ubiquitin (GenBank Accession No. BC000449 (SEQ ID NO:1582); amplicon-Ubiquitin-amplicon) and SDHA (GenBank Accession No. NM_{—}004168 (SEQ ID NO:1583); amplicon—SDHA-amplicon (SEQ ID NO: 1273)) was measured similarly. For each RT sample, the expression of the above amplicon was normalized to the geometric mean of the quantities of the housekeeping genes. The normalized quantity of each RT sample was then divided by the median of the quantities of the ovary samples (Sample Nos. 18-20, Table 2, “Tissue samples on normal panel”), to obtain a value of relative expression of each sample relative to median of the ovary samples.

Forward primer (SEQ ID NO: 1292):
CTCCTGAACCCTACTCCAAGCA
Reverse primer (SEQ ID NO: 1293):
CAGGCGATCCTATGGAAATCC
Amplicon (SEQ ID NO: 1294):
CTCCTGAACCCTACTCCAAGCACAGCCTCTGTCTGACTCCCTTGTCCTTC
AAGAGAACTGTTCTCCAGGTCTCAGGGCCAGGATTTCCATAGGATCGCCT
G

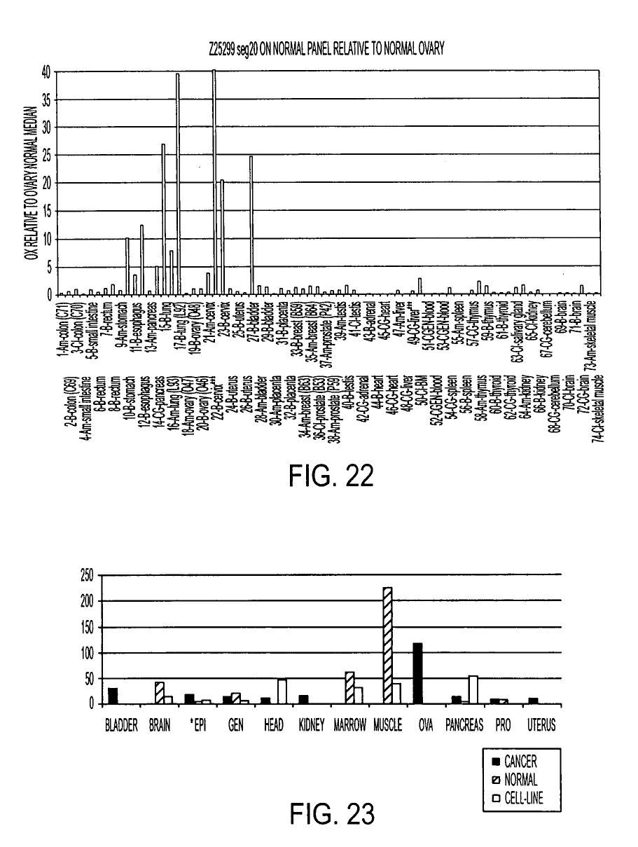

The results are demonstrated in FIG. 22, showing the expression of Secretory leukocyte protease inhibitor Acid-stable proteinase inhibitor with strong affinities for trypsin, chymotrypsin, elastase, and cathepsin G. May prevent elastase-mediated damage to oral and possibly other mucosal tissues Z25299 transcripts which are detectable by amplicon as depicted in sequence name Z25299seg20 (SEQ ID NO: 1294) in different normal tissues.

### Description for Cluster HUMF5A

Cluster HUMF5A features 3 transcript(s) and 33 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| HUMF5A_PEA_1_T1 | 33 |
| HUMF5A_PEA_1_T3 | 34 |
| HUMF5A_PEA_1_T7 | 35 |

TABLE 2
Segments of interest

| Segment Name | SEQ ID NO: |
|---|---|
| HUMF5A_PEA_1_node_0 | 252 |
| HUMF5A_PEA_1_node_4 | 253 |
| HUMF5A_PEA_1_node_6 | 254 |
| HUMF5A_PEA_1_node_8 | 255 |
| HUMF5A_PEA_1_node_10 | 256 |
| HUMF5A_PEA_1_node_12 | 257 |
| HUMF5A_PEA_1_node_14 | 258 |
| HUMF5A_PEA_1_node_18 | 259 |
| HUMF5A_PEA_1_node_21 | 260 |
| HUMF5A_PEA_1_node_22 | 261 |
| HUMF5A_PEA_1_node_24 | 262 |
| HUMF5A_PEA_1_node_26 | 263 |
| HUMF5A_PEA_1_node_27 | 264 |
| HUMF5A_PEA_1_node_29 | 265 |
| HUMF5A_PEA_1_node_35 | 266 |
| HUMF5A_PEA_1_node_37 | 267 |
| HUMF5A_PEA_1_node_39 | 268 |
| HUMF5A_PEA_1_node_47 | 269 |
| HUMF5A_PEA_1_node_50 | 270 |
| HUMF5A_PEA_1_node_53 | 271 |
| HUMF5A_PEA_1_node_56 | 272 |
| HUMF5A_PEA_1_node_60 | 273 |
| HUMF5A_PEA_1_node_2 | 274 |
| HUMF5A_PEA_1_node_16 | 275 |
| HUMF5A_PEA_1_node_31 | 276 |
| HUMF5A_PEA_1_node_32 | 277 |
| HUMF5A_PEA_1_node_33 | 278 |
| HUMF5A_PEA_1_node_41 | 279 |
| HUMF5A_PEA_1_node_43 | 280 |
| HUMF5A_PEA_1_node_45 | 281 |
| HUMF5A_PEA_1_node_51 | 282 |
| HUMF5A_PEA_1_node_57 | 283 |
| HUMF5A_PEA_1_node_59 | 284 |

TABLE 3
Proteins of interest

| | SEQ | |
|---|---|---|
| Protein Name | ID NO: | Corresponding Transcript(s) |
| HUMF5A_PEA_1_P3 | 564 | HUMF5A_PEA_1_T1 (SEQ ID |
| | | NO: 33) |
| HUMF5A_PEA_1_P4 | 565 | HUMF5A_PEA_1_T3 (SEQ ID |
| | | NO: 34) |
| HUMF5A_PEA_1_P8 | 566 | HUMF5A_PEA_1_T7 (SEQ ID |
| | | NO: 35) |

These sequences are variants of the known protein Coagulation factor V precursor (SwissProt accession identifier FA5_HUMAN; known also according to the synonyms Activated protein C cofactor), SEQ ID NO: 626, referred to herein as the previously known protein.

Protein Coagulation factor V precursor (SEQ ID NO:626) is known or believed to have the following function(s): Coagulation factor V is a cofactor that participates with factor Xa to activate prothrombin to thrombin. The sequence for protein Coagulation factor V precursor is given at the end of the application, as “Coagulation factor V precursor amino acid sequence”. Known polymorphisms for this sequence are as shown in Table 4.

TABLE 4
Amino acid mutations for Known Protein

| SNP position(s) | |
|---|---|
| on amino | |
| acid sequence | Comment |
| 107 | D -> H (in dbSNP: 6019). /FTId = VAR_013886. |
| 334 | R -> G (in APCR; Hong Kong). /FTId = VAR_013620. |
| 334 | R -> T (in APCR; Cambridge). /FTId = VAR_013621. |
| 413 | M -> T (in dbSNP: 6033). /FTId = VAR_013887. |
| 513 | R -> K (in dbSNP: 6020). /FTId = VAR_013622. |
| 534 | R -> Q (in APCR; Leiden; dbSNP: 6025). |
| | /FTId = VAR_001213. |
| 809 | P -> S (in dbSNP: 6031). /FTId = VAR_013888. |
| 817 | N -> T (in dbSNP: 6018). /FTId = VAR_013889. |
| 858 | K -> R (in dbSNP: 4524). /FTId = VAR_001214. |
| 865 | H -> R (in dbSNP: 4525). /FTId = VAR_001215. |
| 925 | K -> E (in dbSNP: 6032). /FTId = VAR_013890. |
| 1146 | H -> Q (in dbSNP: 6005). /FTId = VAR_013891. |
| 1285 | L -> I (in dbSNP: 1046712). /FTId = VAR_013892. |
| 1327 | H -> R (in dbSNP: 1800595). /FTId = VAR_013893. |
| 1530 | E -> A (in dbSNP: 6007). /FTId = VAR_013894. |
| 1685 | T -> S (in dbSNP: 6011). /FTId = VAR_013895. |
| 1749 | L -> V (in dbSNP: 6034). /FTId = VAR_013896. |
| 1764 | V -> M (in dbSNP: 6030). /FTId = VAR_013897. |
| 1820 | M -> I (in dbSNP: 6026). /FTId = VAR_013898. |
| 2102 | R -> H (in APCR). /FTId = VAR_017329. |
| 2222 | D -> G (in dbSNP: 6027). /FTId = VAR_013899. |
| 2213 | T -> A |

The following GO Annotation(s) apply to the previously known protein. The following annotation(s) were found: cell adhesion; blood coagulation, which are annotation(s) related to Biological Process; and blood coagulation factor; copper binding, which are annotation(s) related to Molecular Function.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

As noted above, cluster HUMF5A features 3 transcript(s), which were listed in Table 1 above. These transcript(s) encode for protein(s) which are variant(s) of protein Coagulation factor V precursor (SEQ ID NO:626). A description of each variant protein according to the present invention is now provided.

Variant protein HUMF5A_PEA_{—}1_P3 (SEQ ID NO:564) according to the present invention has an amino acid sequence as given at the end of the application; it is encoded by transcript(s) HUMF5A_PEA_{—}1_T1 (SEQ ID NO:33). An alignment is given to the known protein (Coagulation factor V precursor (SEQ ID NO:626)) at the end of the application. One or more alignments to one or more previously published protein sequences are given at the end of the application. A brief description of the relationship of the variant protein according to the present invention to each such aligned protein is as follows:

Comparison Report Between HUMF5A_PEA_{—}1_P3 (SEQ ID NO:564) and FA5_HUMAN_V1 (SEQ ID NO 627):

1. An isolated chimeric polypeptide encoding for HUMF5A_PEA_{—}1_P3 (SEQ ID NO:564), comprising a first amino acid sequence being at least 90% homologous to MFPGCPRLWVLVVLGTSWVGWGSQGTEAAQLRQFYVAAQGISWSYRPEPTNSSLNLSVTSFKKIVYREY EPYFKKEKPQSTISGLLGPTLYAEVGDIIKVHFKNKADKPLSIHPQGIRYSKLSEGASYLDHTFPAEKMDDA VAPGREYTYEWSISEDSGPTHDDPPCLTHIYYSHENLIEDFNSGLIGPLLICKKGTLTEGGTQKTFDKQIVLL FAVFDESKSWSQSSSLMYTVNGYVNGTMPDITVCAHDHISWHLLGMSSGPELFSIHFNGQVLEQNHHKVS AITLVSATSTTANMTVGPEGKWIISSLTPKHLQAGMQAYIDIKNCPKKTRNLKKITREQRREMKRWEYFIA AEEVIWDYAPVIPANMDKKYRSQHLDNFSNQIGKHYKKVMYTQYEDESFTKHTVNPNMKEDGILGPIIRA QVRDTLKIVFKNMASRPYSIYPHGVTFSPYEDEVNSSFTSGRNNTMIRAVQPGETYTYKWNILEFDEPTEN DAQCLTRPYYSDVDIMRDIASGLIGLLLICKSRSLDRRGIQRAADIEQQAVFAVFDENKSWYLEDNINKFCE NPDEVKRDDPKFYESNIMSTINGYVPESITTLGFCFDDTVQWHFCSVGTQNEILTIHFTGHSFIYGKRHEDTL TLFPMRGESVTVTMDNVGTWMLTSMNSSPRSKKLRLKFRDVKCIPDDDEDSYEIFEPPESTVMATRKMHD RLEPEDEESDADYDYQNRLAAALGIRSFRNSSLNQEEEEFNLTALALENGTEFVSSNTDIIVGSNYSSPSNIS KFTVNNLAEPQKAPSHQQATTAGSPLRHLIGKNSVLNSSTAEHSSPYSEDPIEDPLQPDVTGIRLLSLGAGEF RSQEHAKRKGPKVERDQAAKHRFSWMKLLAHKVGRHLSQDTGSPSGMRPWEDLPSQDTGSPSRMRPWK DPPSDLLLLKQSNSSKILVGRWHLASEKGSYEIIQDTDEDTAVNNWLISPQNASRAWGESTPLANKPGKQS GHPKFPRVRHKSLQVRQDGGKSRLKKSQFLIKTRKKKKEKHTHHAPLSPRTFHPLRSEAYNTFSERRLKHS LVLHKSNETSLPTDLNQTLPSMDFGWIASLPDHNQNSSNDTGQASCPPGLYQTVPPEEHYQTFPIQDPDQM HSTSDPSHRSSSPELSEMLEYDRSHKSFPTDISQMSPSSEHEVWQTVISPDLSQVTLSPELSQTNLSPDLSHTT LSPELIQRNLSPALGQMPISPDLSHTTLSPDLSHTTLSLDLSQTNLSPELSQTNLSPALGQMPLSPDLSHTTLS LDFSQTNLSPELSHMTLSPELSQTNLSPALGQMPISPDLSHTTLSLDFSQTNLSPELSQTNLSPALGQMPLSP DPSHTTLSLDLSQTNLSPELSQTNLSPDLSEMPLFADLSQIPLTPDLDQMTLSPDLGETDLSPNFGQMSLSPD LSQVTLSPDISDTTLLPDLSQISPPPDLDQIFYPSESSQSLLLQEFNESFPYPDLGQMPSPSSPTLNDTFLSKEF NPLVIVGLSKDGTDYIEIIPKEEVQSSEDDYAEIDYVPYDDPYKTDVRTNINSSRDPDNIAAWYLRSNNGNR RNYYIAAEEISWDYSEFVQRETDIEDSDDIPEDTTYKK corresponding to amino acids 1-1617 of FA5_HUMAN_V1 (SEQ ID NO:627), which also corresponds to amino acids 1-1617 of HUMF5A_PEA_{—}1_P3 (SEQ ID NO:564), and a second amino acid sequence being at least 70%, optionally at least 80%, preferably at least 85%, more preferably at least 90% and most preferably at least 95% homologous to a polypeptide having the sequence GSMKSISEFLVLLSELKWMMLSKFVLKI (SEQ ID NO:1506) corresponding to amino acids 1618-1645 of HUMF5A_PEA_{—}1_P3 (SEQ ID NO:564), wherein said first amino acid sequence and second amino acid sequence are contiguous and in a sequential order.

2. An isolated polypeptide encoding for a tail of HUMF5A_PEA_{—}1_P3 (SEQ ID NO:564), comprising a polypeptide being at least 70%, optionally at least about 80%, preferably at least about 85%, more preferably at least about 90% and most preferably at least about 95% homologous to the sequence GSMKSISEFLVLLSELKWMMLSKFVLKI (SEQ ID NO:1506) in HUMF5A_PEA_{—}1_P3 (SEQ ID NO:564).

It should be noted that the known protein sequence (FA5_HUMAN (SEQ ID NO:626)) has one or more changes than the sequence given at the end of the application and named as being the amino acid sequence for FA5_HUMAN_V1 (SEQ ID NO:627). These changes were previously known to occur and are listed in the table below.

TABLE 5
Changes to FA5_HUMAN_V1 (SEQ ID NO: 627)

| SNP position(s) on amino | |
|---|---|
| acid sequence | Type of change |
| 859 | variant |
| 866 | variant |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMF5A_PEA_{—}1_P3 (SEQ ID NO:564) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein HUMF5A_PEA_{—}1_P3 (SEQ ID NO:564) sequence provides support for the deduced sequence of this variant protein according to the present invention).

TABLE 6
Amino acid mutations

| SNP position(s) on amino acid | Alternative | |
|---|---|---|
| sequence | amino acid(s) | Previously known SNP? |
| 15 | G -> S | Yes |
| 107 | D -> H | Yes |
| 413 | M -> T | Yes |
| 513 | R -> K | Yes |
| 534 | R -> Q | Yes |
| 781 | S -> R | Yes |
| 809 | P -> S | Yes |
| 817 | N -> T | Yes |
| 858 | R -> K | Yes |
| 865 | R -> H | Yes |
| 915 | T -> S | Yes |
| 925 | K -> E | Yes |
| 969 | N -> S | Yes |
| 980 | R -> L | Yes |
| 1146 | H -> Q | Yes |
| 1169 | D -> | No |
| 1285 | L -> I | Yes |
| 1327 | H -> R | Yes |
| 1397 | L -> F | Yes |
| 1404 | P -> S | Yes |
| 1530 | E -> A | Yes |

Variant protein HUMF5A_PEA_{—}1_P3 (SEQ ID NO:564) is encoded by the following transcript(s): HUMF5A_PEA_{—}1_T1 (SEQ ID NO:33), for which the sequence(s) is/are given at the end of the application. The coding portion of transcript HUMF5A_PEA_{—}1_T1 (SEQ ID NO:33) is shown in bold; this coding portion starts at position 183 and ends at position 5117. The transcript also has the following SNPs as listed in Table 7 (given according to their position on the nucleotide sequence, with the alternative nucleic acid listed; the last column indicates whether the SNP is known or not; the presence of known SNPs in variant protein HUMF5A_PEA_{—}1_P3 (SEQ ID NO:564) sequence provides support for the deduced sequence of this variant protein according to the present invention).

TABLE 7
Nucleic acid SNPs

| SNP position on | Alternative | Previously |
|---|---|---|
| nucleotide sequence | nucleic acid | known SNP? |
| 16 | C -> T | Yes |
| 225 | G -> A | Yes |
| 419 | A -> G | Yes |
| 501 | G -> C | Yes |
| 587 | G -> A | Yes |
| 734 | G -> T | Yes |
| 746 | G -> C | Yes |
| 951 | C -> T | Yes |
| 998 | C -> T | Yes |
| 1420 | T -> C | Yes |
| 1424 | A -> G | Yes |
| 1562 | C -> T | Yes |
| 1720 | G -> A | Yes |
| 1783 | G -> A | Yes |
| 1898 | G -> A | Yes |
| 2102 | C -> T | Yes |
| 2108 | C -> A | Yes |
| 2390 | T -> C | Yes |
| 2417 | C -> T | Yes |
| 2471 | A -> G | Yes |
| 2483 | G -> A | Yes |
| 2525 | T -> G | Yes |
| 2607 | C -> T | Yes |
| 2632 | A -> C | Yes |
| 2755 | G -> A | Yes |
| 2776 | G -> A | Yes |
| 2925 | A -> T | Yes |
| 2955 | A -> G | Yes |
| 3088 | A -> G | Yes |
| 3121 | G -> T | Yes |
| 3437 | A -> G | Yes |
| 3620 | C -> G | Yes |
| 3686 | A -> C | Yes |
| 3688 | A -> | No |
| 3689 | T -> | No |
| 3764 | C -> T | Yes |
| 3986 | T -> C | Yes |
| 4035 | C -> A | Yes |
| 4130 | C -> T | Yes |
| 4162 | A -> G | Yes |
| 4277 | C -> T | Yes |
| 4371 | C -> T | Yes |
| 4392 | C -> T | Yes |
| 4771 | A -> C | Yes |
| 5152 | A -> G | Yes |
| 5184 | C -> G | Yes |
| 5375 | C -> G | Yes |
| 5420 | G -> A | Yes |
| 5590 | G -> A | Yes |
| 6573 | T -> C | Yes |
| 6684 | A -> G | Yes |
| 6795 | A -> G | Yes |

Variant protein HUMF5A_PEA_{—}1_P4 (SEQ ID NO:565) according to the present invention has an amino acid sequence as given at the end of the application; it is encoded by transcript(s) HUMF5A_PEA_{—}1_T3 (SEQ ID NO:34). An alignment is given to the known protein (Coagulation factor V precursor (SEQ ID NO:626)) at the end of the application. One or more alignments to one or more previously published protein sequences are given at the end of the application. A brief description of the relationship of the variant protein according to the present invention to each such aligned protein is as follows:

Comparison Report Between HUMF5A_PEA_{—}1_P4 (SEQ ID NO:565) and FA5_HUMAN_V1 (SEQ ID NO:627):

1. An isolated chimeric polypeptide encoding for HUMF5A_PEA_{—}1_P4 (SEQ ID NO:565), comprising a first amino acid sequence being at least 90% homologous to MFPGCPRLWVLVVLGTSWVGWGSQGTEAAQLRQFYVAAQGISWSYRPEPTNSSLNLSVTSFKKIVYREY EPYFKKEKPQSTISGLLGPTLYAEVGDIIKVHFKNKADKPLSIHPQGIRYSKLSEGASYLDHTFPAEKMDDA VAPGREYTYEWSISEDSGPTHDDPPCLTHIYYSHENLIEDFNSGLIGPLLICKKGTLTEGGTQKTFDKQIVLL FAVFDESKSWSQSSSLMYTVNGYVNGTMPDITVCAHDHISWHLLGMSSGPELFSIHFNGQVLEQNHHKVS AITLVSATSTTANMTVGPEGKWIISSLTPKHLQAGMQAYIDIKNCPKKTRNLKKITREQRRHMKRWEYFIA AEEVIWDYAPVIPANMDKKYRSQHLDNFSNQIGKHYKKVMYTQYEDESFTKHTVNPNMKEDGILGPIIRA QVRDTLKIVFKNMASRPYSIYPHGVTFSPYEDEVNSSFTSGRNNTMIRAVQPGETYTYKWNILEFDEPTEN DAQCLTRPYYSDVDIMRDIASGLIGLLLICKSRSLDRRGIQRAADIEQQAVFAVFDENKSWYLEDNINKFCE NPDEVKRDDPKFYESNIMSTINGYVPESITTLGFCFDDTVQWHFCSVGTQNEILTIHFTGHSFIYGKRHEDTL TLFPMRGESVTVTMDNVGTWMLTSMNSSPRSKKLRLKFRDVKCIPDDDEDSYEIFEPPESTVMATRKMHD RLEPEDEESDADYDYQNRLAAALGIRSFRNSSLNQEEEEFNLTALALENGTEFVSSNTDIIVGSNYSSPSNIS KFTVNNLAEPQKAPSHQQATTAGSPLRHLIGKNSVLNSSTAEHSSPYSEDPIEDPLQPDVTGIRLLSLGAGEF RSQEHAKRKGPKVERDQAAKHRFSWMKLLAHKVGRHLSQDTGSPSGMRPWEDLPSQDTGSPSRMRPWK DPPSDLLLLKQSNSSKILVGRWHLASEKGSYEIIQDTDEDTAVNNWLISPQNASRAWGESTPLANKPGKQS GHPKFPRVRHKSLQVRQDGGKSRLKKSQFLIKTRKKKKEKHTHHAPLSPRTFHPLRSEAYNTFSERRLKHS LVLHKSNETSLPTDLNQTLPSMDFGWIASLPDHNQNSSNDTGQASCPPGLYQTVPPEEHYQTFPIQDPDQM HSTSDPSHRSSSPELSEMLEYDRSHKSFPTDISQMSPSSEHEVWQTVISPDLSQVTLSPELSQTNLSPDLSHTT LSPELIQRNLSPALGQMPISPDLSHTTLSPDLSHTTLSLDLSQTNLSPELSQTNLSPALGQMPLSPDLSHTTLS LDFSQTNLSPELSHMTLSPELSQTNLSPALGQMPISPDLSHTTLSLDFSQTNLSPELSQTNLSPALGQMPLSP DPSHTTLSLDLSQTNLSPELSQTNLSPDLSEMPLFADLSQIPLTPDLDQMTLSPDLGETDLSPNFGQMSLSPD LSQVTLSPDISDTTLLPDLSQISPPPDLDQIFYPSESSQSLLLQEFNESFPYPDLGQMPSPSSPTLNDTFLSKEF NPLVIVGLSKDGTDYIEIIPKEEVQSSEDDYAEIDYVPYDDPYKTDVRTNINSSRDPDNIAAWYLRSNNGNR RNYYIAAEEISWDYSEFVQRETDIEDSDDIPEDTTYKKVVFRKYLDSTFTKRDPRGEYEEHLGILGPIIRAEV DDVIQVRFKNLASRPYSLHAHGLSYEKSSEGKTYEDDSPEWFKEDNAVQPNSSYTYVWHATERSGPESPG SACRAWAYYSAVNPEKDIHSGLIGPLLICQKGILHKDSNMPVDMREFVLLFMTFDEKKSWYYEKKSRSSW RLTSSEMKKSHEFHAINGMIYSLPGLKMYEQEWVRLHLLNIGGSQDIHVVHFHGQTLLENGNKQHQLGV WPLLPGSFKTLEMKASKPGWWLLNTEVGENQRAGMQTPFLIMDRDCRMPMGLSTGIISDSQIKASEFLGY WEPRLARLNNGGSYNAWSVEKLAAEFASKPWIQVDMQKEVIITGIQTQGAKHYLKSCYTTEFYVAYSSN QINWQIFKGNSTRNVMYFNGNSDASTIKENQFDPPIVARYIRISPTRAYNRPTLRLELQGCE corresponding to amino acids 1-2062 of FA5_HUMAN_V1 (SEQ ID NO:627), which also corresponds to amino acids 1-2062 of HUMF5A_PEA_{—}1_P4 (SEQ ID NO:565), and a second amino acid sequence being at least 70%, optionally at least 80%, preferably at least 85%, more preferably at least 90% and most preferably at least 95% homologous to a polypeptide having the sequence DVPHPWVWKMER (SEQ ID NO:1507) corresponding to amino acids 2063-2074 of HUMF5A_PEA_{—}1_P4 (SEQ ID NO:565), wherein said first amino acid sequence and second amino acid sequence are contiguous and in a sequential order.

2. An isolated polypeptide encoding for a tail of HUMF5A_PEA_{—}1_P4 (SEQ ID NO:565), comprising a polypeptide being at least 70%, optionally at least about 80%, preferably at least about 85%, more preferably at least about 90% and most preferably at least about 95% homologous to the sequence DVPHPWVWKMER (SEQ ID NO:1507) in HUMF5A_PEA_{—}1_P4 (SEQ ID NO:565).

It should be noted that the known protein sequence (FA5_HUMAN (SEQ ID NO:626)) has one or more changes than the sequence given at the end of the application and named as being the amino acid sequence for FA5_HUMAN_V1 (SEQ ID NO:627). These changes were previously known to occur and are listed in the table below.

TABLE 8
Changes to FA5_HUMAN_V1 (SEQ ID NO: 627)

| SNP position(s) on amino | |
|---|---|
| acid sequence | Type of change |
| 859 | variant |
| 866 | variant |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMF5A_PEA_{—}1_P4 (SEQ ID NO:565) also has the following non-silent SNPs (Single Nucleotide Polymorphisms) as listed in Table 9, (given according to their position(s) on the amino acid sequence, with the alternative amino acid(s) listed; the last column indicates whether the SNP is known or not; the presence of known SNPs in variant protein HUMF5A_PEA_{—}1_P4 (SEQ ID NO:565) sequence provides support for the deduced sequence of this variant protein according to the present invention).

TABLE 9
Amino acid mutations

| SNP position(s) on amino acid | Alternative | |
|---|---|---|
| sequence | amino acid(s) | Previously known SNP? |
| 15 | G -> S | Yes |
| 107 | D -> H | Yes |
| 413 | M -> T | Yes |
| 513 | R -> K | Yes |
| 534 | R -> Q | Yes |
| 781 | S -> R | Yes |
| 809 | P -> S | Yes |
| 817 | N -> T | Yes |
| 858 | R -> K | Yes |
| 865 | R -> H | Yes |
| 915 | T -> S | Yes |
| 925 | K -> E | Yes |
| 969 | N -> S | Yes |
| 980 | R -> L | Yes |
| 1146 | H -> Q | Yes |
| 1169 | D -> | No |
| 1285 | L -> I | Yes |
| 1327 | H -> R | Yes |
| 1397 | L -> F | Yes |
| 1404 | P -> S | Yes |
| 1530 | E -> A | Yes |
| 1685 | T -> S | Yes |
| 1749 | L -> V | Yes |
| 1764 | V -> M | Yes |
| 1820 | M -> I | Yes |

Variant protein HUMF5A_PEA_{—}1_P4 (SEQ ID NO:565) is encoded by the following transcript(s): HUMF5A_PEA_{—}1_T3 (SEQ ID NO:34), for which the sequence(s) is/are given at the end of the application. The coding portion of transcript HUMF5A_PEA_{—}1_T3 (SEQ ID NO:34) is shown in bold; this coding portion starts at position 183 and ends at position 6404. The transcript also has the following SNPs as listed in Table 10 (given according to their position on the nucleotide sequence, with the alternative nucleic acid listed; the last column indicates whether the SNP is known or not; the presence of known SNPs in variant protein HUMF5A_PEA_{—}1_P4 (SEQ ID NO:565) sequence provides support for the deduced sequence of this variant protein according to the present invention).

TABLE 10
Nucleic acid SNPs

| SNP position on | Alternative | Previously |
|---|---|---|
| nucleotide sequence | nucleic acid | known SNP? |
| 16 | C -> T | Yes |
| 225 | G -> A | Yes |
| 419 | A -> G | Yes |
| 501 | G -> C | Yes |
| 587 | G -> A | Yes |
| 734 | G -> T | Yes |
| 746 | G -> C | Yes |
| 951 | C -> T | Yes |
| 998 | C -> T | Yes |
| 1420 | T -> C | Yes |
| 1424 | A -> G | Yes |
| 1562 | C -> T | Yes |
| 1720 | G -> A | Yes |
| 1783 | G -> A | Yes |
| 1898 | G -> A | Yes |
| 2102 | C -> T | Yes |
| 2108 | C -> A | Yes |
| 2390 | T -> C | Yes |
| 2417 | C -> T | Yes |
| 2471 | A -> G | Yes |
| 2483 | G -> A | Yes |
| 2525 | T -> G | Yes |
| 2607 | C -> T | Yes |
| 2632 | A -> C | Yes |
| 2755 | G -> A | Yes |
| 2776 | G -> A | Yes |
| 2925 | A -> T | Yes |
| 2955 | A -> G | Yes |
| 3088 | A -> G | Yes |
| 3121 | G -> T | Yes |
| 3437 | A -> G | Yes |
| 3620 | C -> G | Yes |
| 3686 | A -> C | Yes |
| 3688 | A -> | No |
| 3689 | T -> | No |
| 3764 | C -> T | Yes |
| 3986 | T -> C | Yes |
| 4035 | C -> A | Yes |
| 4130 | C -> T | Yes |
| 4162 | A -> G | Yes |
| 4277 | C -> T | Yes |
| 4371 | C -> T | Yes |
| 4392 | C -> T | Yes |
| 4771 | A -> C | Yes |
| 5204 | A -> G | Yes |
| 5236 | C -> G | Yes |
| 5427 | C -> G | Yes |
| 5472 | G -> A | Yes |
| 5642 | G -> A | Yes |
| 6618 | T -> C | Yes |
| 6729 | A -> G | Yes |
| 6840 | A -> G | Yes |

Variant protein HUMF5A_PEA_{—}1_P8 (SEQ ID NO:566) according to the present invention has an amino acid sequence as given at the end of the application; it is encoded by transcript(s) HUMF5A_PEA_{—}1_T7 (SEQ ID NO:35). An alignment is given to the known protein (Coagulation factor V precursor (SEQ ID NO:626)) at the end of the application. One or more alignments to one or more previously published protein sequences are given at the end of the application. A brief description of the relationship of the variant protein according to the present invention to each such aligned protein is as follows:

Comparison Report Between HUMF5A_PEA_{—}1_P8 (SEQ ID NO:566) and FA5_HUMAN (SEQ ID NO:626):

1. An isolated chimeric polypeptide encoding for HUMF5A_PEA_{—}1_P8 (SEQ ID NO:566), comprising a first amino acid sequence being at least 90% homologous to MFPGCPRLWVLVVLGTSWVGWGSQGTEAAQLRQFYVAAQGISWSYRPEPTNSSLNLSVTSFKKIVYREY EPYFKKEKPQSTISGLLGPTLYAEVGDIIKVHFKNKADKPLSIHPQGIRYSKLSEGASYLDHTFPAEKMDDA VAPGREYTYEWSISEDSGPTHDDPPCLTHIYYSHENLIEDFNSGLIGPLLICKKGTLTEGGTQKTFDKQIVLL FAVFDESKSWSQSSSLMYTVNGYVNGTMPDITVCAHDHISWHLLGMSSGPELFSIHFNGQVLEQNHHKVS AITLVSATSTTANMTVGPEGKWIISSLTPKHLQAGMQAYIDIKNCPKKTRNLKKITREQRRHMKRWEYFIA AEEVIWDYAPVIPANMDKKYRSQHLDNFSNQIGKHYKKVMYTQYEDESFTKHTVNPNMKEDGILGPIIRA QVRDTLKIVFKNMASRPYSIYPHGVTFSPYEDEVNSSFTSGRNNTMIRAVQPGETYTYKWNILEFDEPTEN DAQCLTRPYYSDVDIMRDIASGLIGLLLICKSRSLDRRGIQRAADIEQQAVFAVFDENKSWYLEDNINKFCE NPDEVKRDDPKFYESNIMS corresponding to amino acids 1-587 of FA5_HUMAN (SEQ ID NO:626), which also corresponds to amino acids 1-587 of HUMF5A_PEA_{—}1_P8 (SEQ ID NO:566), and a second amino acid sequence being at least 70%, optionally at least 80%, preferably at least 85%, more preferably at least 90% and most preferably at least 95% homologous to a polypeptide having the sequence SKSEYYFCSSVFHSCG (SEQ ID NO:1508) corresponding to amino acids 588-603 of HUMF5A_PEA_{—}1_P8 (SEQ ID NO:566), wherein said first amino acid sequence and second amino acid sequence are contiguous and in a sequential order.

2. An isolated polypeptide encoding for a tail of HUMF5A_PEA_{—}1_P8 (SEQ ID NO:566), comprising a polypeptide being at least 70%, optionally at least about 80%, preferably at least about 85%, more preferably at least about 90% and most preferably at least about 95% homologous to the sequence SKSEYYFCSSVFHSCG (SEQ ID NO:1508) in HUMF5A_PEA_{—}1_P8 (SEQ ID NO:566).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMF5A_PEA_{—}1_P8 (SEQ ID NO:566) also has the following non-silent SNPs (Single Nucleotide Polymorphisms) as listed in Table 11, (given according to their position(s) on the amino acid sequence, with the alternative amino acid(s) listed; the last column indicates whether the SNP is known or not; the presence of known SNPs in variant protein HUMF5A_PEA_{—}1_P8 (SEQ ID NO:566) sequence provides support for the deduced sequence of this variant protein according to the present invention).

TABLE 11
Amino acid mutations

| SNP position(s) on amino acid | Alternative | |
|---|---|---|
| sequence | amino acid(s) | Previously known SNP? |
| 15 | G -> S | Yes |
| 107 | D -> H | Yes |
| 413 | M -> T | Yes |
| 513 | R -> K | Yes |
| 534 | R -> Q | Yes |

The glycosylation sites of variant protein HUMF5A_PEA_{—}1_P8 (SEQ ID NO:566), as compared to the known protein Coagulation factor V precursor (SEQ ID NO:626), are described in Table 12 (given according to their position(s) on the amino acid sequence in the first column; the second column indicates whether the glycosylation site is present in the variant protein; and the last column indicates whether the position is different on the variant protein).

TABLE 12
Glycosylation site(s)

| Position(s) on known amino | Present in | Position |
|---|---|---|
| acid sequence | variant protein? | in variant protein? |
| 821 | no | |
| 554 | yes | 554 |
| 1703 | no | |
| 741 | no | |
| 55 | yes | 55 |
| 297 | yes | 297 |
| 752 | no | |
| 468 | yes | 468 |
| 460 | yes | 460 |
| 1559 | no | |
| 782 | no | |
| 1479 | no | |
| 938 | no | |
| 776 | no | |
| 760 | no | |
| 1103 | no | |
| 1499 | no | |
| 1106 | no | |
| 977 | no | |
| 2010 | no | |
| 239 | yes | 239 |
| 1074 | no | |
| 2209 | no | |
| 1083 | no | |
| 51 | yes | 51 |
| 382 | yes | 382 |

The phosphorylation sites of variant protein HUMF5A_PEA_{—}1_P8 (SEQ ID NO:566), as compared to the known protein Coagulation factor V precursor (SEQ ID NO:626), are described in Table 13 (given according to their position(s) on the amino acid sequence in the first column; the second column indicates whether the phosphorylation site is present in the variant protein; and the last column indicates whether the position is different on the variant protein).

TABLE 13
Phosphorylation site(s)

| Position(s) on known amino acid | |
|---|---|
| sequence | Present in variant protein? |
| 724 | no |
| 726 | no |
| 1543 | no |
| 1538 | no |
| 693 | no |
| 1593 | no |
| 1522 | no |

Variant protein HUMF5A_PEA_{—}1_P8 (SEQ ID NO:566) is encoded by the following transcript(s): HUMF5A_PEA_{—}1_T7 (SEQ ID NO:35), for which the sequence(s) is/are given at the end of the application. The coding portion of transcript HUMF5A_PEA_{—}1_T7 (SEQ ID NO:35) is shown in bold; this coding portion starts at position 183 and ends at position 1991. The transcript also has the following SNPs as listed in Table 14 (given according to their position on the nucleotide sequence, with the alternative nucleic acid listed; the last column indicates whether the SNP is known or not; the presence of known SNPs in variant protein HUMF5A_PEA_{—}1_P8 (SEQ ID NO:566) sequence provides support for the deduced sequence of this variant protein according to the present invention).

TABLE 14
Nucleic acid SNPs

| SNP position on | Alternative | Previously |
|---|---|---|
| nucleotide sequence | nucleic acid | known SNP? |
| 16 | C -> T | Yes |
| 225 | G -> A | Yes |
| 419 | A -> G | Yes |
| 501 | G -> C | Yes |
| 587 | G -> A | Yes |
| 734 | G -> T | Yes |
| 746 | G -> C | Yes |
| 951 | C -> T | Yes |
| 998 | C -> T | Yes |
| 1420 | T -> C | Yes |
| 1424 | A -> G | Yes |
| 1562 | C -> T | Yes |
| 1720 | G -> A | Yes |
| 1783 | G -> A | Yes |
| 1898 | G -> A | Yes |
| 2088 | G -> A | Yes |
| 2095 | G -> A | Yes |

As noted above, cluster HUMF5A features 33 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HUMF5A_PEA_{—}1_node_{—}0 (SEQ ID NO:252) according to the present invention is supported by 9 libraries. The number of libraries was determined as previously described. This segment can be found in the following transcript(s): HUMF5A_PEA_{—}1_T1 (SEQ ID NO:33), HUMF5A_PEA_{—}1_T3 (SEQ ID NO:34) and HUMF5A_PEA_{—}1_T7 (SEQ ID NO:35). Table 15 below describes the starting and ending position of this segment on each transcript.

TABLE 15
Segment location on transcripts

| | Segment | |
|---|---|---|
| | starting | Segment ending |
| Transcript name | position | position |
| HUMF5A_PEA_1_T1 (SEQ ID NO: 33) | 1 | 340 |
| HUMF5A_PEA_1_T3 (SEQ ID NO: 34) | 1 | 340 |
| HUMF5A_PEA_1_T7 (SEQ ID NO: 35) | 1 | 340 |

Segment cluster HUMF5A_PEA_{—}1_node_{—}4 (SEQ ID NO:253) according to the present invention is supported by 7 libraries. The number of libraries was determined as previously described. This segment can be found in the following transcript(s): HUMF5A_PEA_{—}1_T1 (SEQ ID NO:33), HUMF5A_PEA_{—}1_T3 (SEQ ID NO:34) and HUMF5A_PEA_{—}1_T7 (SEQ ID NO:35). Table 16 below describes the starting and ending position of this segment on each transcript.

TABLE 16
Segment location on transcripts

| | Segment | |
|---|---|---|
| | starting | Segment ending |
| Transcript name | position | position |
| HUMF5A_PEA_1_T1 (SEQ ID NO: 33) | 433 | 555 |
| HUMF5A_PEA_1_T3 (SEQ ID NO: 34) | 433 | 555 |
| HUMF5A_PEA_1_T7 (SEQ ID NO: 35) | 433 | 555 |

Segment cluster HUMF5A_PEA_{—}1_node_{—}6 (SEQ ID NO:254) according to the present invention is supported by 11 libraries. The number of libraries was determined as previously described. This segment can be found in the following transcript(s): HUMF5A_PEA_{—}1_T1 (SEQ ID NO:33), HUMF5A_PEA_{—}1_T3 (SEQ ID NO:34) and HUMF5A_PEA_{—}1_T7 (SEQ ID NO:35). Table 17 below describes the starting and ending position of this segment on each transcript.

TABLE 17
Segment location on transcripts

| | Segment | |
|---|---|---|
| | starting | Segment ending |
| Transcript name | position | position |
| HUMF5A_PEA_1_T1 (SEQ ID NO: 33) | 556 | 768 |
| HUMF5A_PEA_1_T3 (SEQ ID NO: 34) | 556 | 768 |
| HUMF5A_PEA_1_T7 (SEQ ID NO: 35) | 556 | 768 |

Segment cluster HUMF5A_PEA_{—}1_node_{—}8 (SEQ ID NO:255) according to the present invention is supported by 8 libraries. The number of libraries was determined as previously described. This segment can be found in the following transcript(s): HUMF5A_PEA_{—}1_T1 (SEQ ID NO:33), HUMF5A_PEA_{—}1_T3 (SEQ ID NO:34) and HUMF5A_PEA_{—}1_T7 (SEQ ID NO:35). Table 18 below describes the starting and ending position of this segment on each transcript.

TABLE 18
Segment location on transcripts

| | Segment | |
|---|---|---|
| | starting | Segment ending |
| Transcript name | position | position |
| HUMF5A_PEA_1_T1 (SEQ ID NO: 33) | 769 | 912 |
| HUMF5A_PEA_1_T3 (SEQ ID NO: 34) | 769 | 912 |
| HUMF5A_PEA_1_T7 (SEQ ID NO: 35) | 769 | 912 |

Segment cluster HUMF5A_PEA_{—}1_node_{—}10 (SEQ ID NO:256) according to the present invention is supported by 7 libraries. The number of libraries was determined as previously described. This segment can be found in the following transcript(s): HUMF5A_PEA_{—}1_T1 (SEQ ID NO:33) HUMF5A_PEA_{—}1_T3 (SEQ ID NO:34) and HUMF5A_PEA_{—}1_T7 (SEQ ID NO:35). Table 19 below describes the starting and ending position of this segment on each transcript.

TABLE 19
Segment location on transcripts

| | Segment | |
|---|---|---|
| | starting | Segment ending |
| Transcript name | position | position |
| HUMF5A_PEA_1_T1 (SEQ ID NO: 33) | 913 | 1134 |
| HUMF5A_PEA_1_T3 (SEQ ID NO: 34) | 913 | 1134 |
| HUMF5A_PEA_1_T7 (SEQ ID NO: 35) | 913 | 1134 |

Segment cluster HUMF5A_PEA_{—}1_node_{—}12 (SEQ ID NO:257) according to the present invention is supported by 10 libraries. The number of libraries was determined as previously described. This segment can be found in the following transcript(s): HUMF5A_PEA_{—}1_T1 (SEQ ID NO:33), HUMF5A_PEA_{—}1_T3 (SEQ ID NO:34) and HUMF5A_PEA_{—}1_T7 (SEQ ID NO:35). Table 20 below describes the starting and ending position of this segment on each transcript.

TABLE 20
Segment location on transcripts

| | Segment | |
|---|---|---|
| | starting | Segment ending |
| Transcript name | position | position |
| HUMF5A_PEA_1_T1 (SEQ ID NO: 33) | 1135 | 1300 |
| HUMF5A_PEA_1_T3 (SEQ ID NO: 34) | 1135 | 1300 |
| HUMF5A_PEA_1_T7 (SEQ ID NO: 35) | 1135 | 1300 |

Segment cluster HUMF5A_PEA_{—}1_node_{—}14 (SEQ ID NO:258) according to the present invention is supported by 9 libraries. The number of libraries was determined as previously described. This segment can be found in the following transcript(s): HUMF5A_PEA_{—}1_T1 (SEQ ID NO:33), HUMF5A_PEA_{—}1_T3 (SEQ ID NO:34) and HUMF5A_PEA_{—}1_T7 (SEQ ID NO:35). Table 21 below describes the starting and ending position of this segment on each transcript.

TABLE 21
Segment location on transcripts

| | Segment | |
|---|---|---|
| | starting | Segment ending |
| Transcript name | position | position |
| HUMF5A_PEA_1_T1 (SEQ ID NO: 33) | 1301 | 1478 |
| HUMF5A_PEA_1_T3 (SEQ ID NO: 34) | 1301 | 1478 |
| HUMF5A_PEA_1_T7 (SEQ ID NO: 35) | 1301 | 1478 |

Segment cluster HUMF5A_PEA_{—}1_node_{—}18 (SEQ ID NO:259) according to the present invention is supported by 10 libraries. The number of libraries was determined as previously described. This segment can be found in the following transcript(s): HUMF5A_PEA_{—}1_T1 (SEQ ID NO:33), HUMF5A_PEA_{—}1_T3 (SEQ ID NO:34) and HUMF5A_PEA_{—}1_T7 (SEQ ID NO:35). Table 22 below describes the starting and ending position of this segment on each transcript.

TABLE 22
Segment location on transcripts

| | Segment | |
|---|---|---|
| | starting | Segment ending |
| Transcript name | position | position |
| HUMF5A_PEA_1_T1 (SEQ ID NO: 33) | 1579 | 1793 |
| HUMF5A_PEA_1_T3 (SEQ ID NO: 34) | 1579 | 1793 |
| HUMF5A_PEA_1_T7 (SEQ ID NO: 35) | 1579 | 1793 |

Segment cluster HUMF5A_PEA_{—}1_node_{—}21 (SEQ ID NO:260) according to the present invention is supported by 12 libraries. The number of libraries was determined as previously described. This segment can be found in the following transcript(s): HUMF5A_PEA_{—}1_T1 (SEQ ID NO:33), HUMF5A_PEA_{—}1_T3 (SEQ ID NO:34) and HUMF5A_PEA_{—}1_T7 (SEQ ID NO:35). Table 23 below describes the starting and ending position of this segment on each transcript.

TABLE 23
Segment location on transcripts

| | Segment | |
|---|---|---|
| | starting | Segment ending |
| Transcript name | position | position |
| HUMF5A_PEA_1_T1 (SEQ ID NO: 33) | 1794 | 1944 |
| HUMF5A_PEA_1_T3 (SEQ ID NO: 34) | 1794 | 1944 |
| HUMF5A_PEA_1_T7 (SEQ ID NO: 35) | 1794 | 1944 |

Segment cluster HUMF5A_PEA_{—}1_node_{—}22 (SEQ ID NO:261) according to the present invention is supported by 1 libraries. The number of libraries was determined as previously described. This segment can be found in the following transcript(s): HUMF5A_PEA_{—}1_T7 (SEQ ID NO:35). Table 24 below describes the starting and ending position of this segment on each transcript.

TABLE 24
Segment location on transcripts

| | Segment | |
|---|---|---|
| | starting | Segment ending |
| Transcript name | position | position |
| HUMF5A_PEA_1_T7 (SEQ ID NO: 35) | 1945 | 2097 |

Segment cluster HUMF5A_PEA_{—}1_node_{—}24 (SEQ ID NO:262) according to the present invention is supported by 13 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 25 below describes the starting and ending position of this segment on each transcript.

TABLE 25
Segment location on transcripts

| | Segment | |
|---|---|---|
| | starting | Segment ending |
| Transcript name | position | position |
| HUMF5A_PEA_1_T1 (SEQ ID NO: 33) | 1945 | 2157 |
| HUMF5A_PEA_1_T3 (SEQ ID NO: 34) | 1945 | 2157 |

Segment cluster HUMF5A_PEA_{—}1_node_{—}26 (SEQ ID NO:263) according to the present invention is supported by 33 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 26 below describes the starting and ending position of this segment on each transcript.

TABLE 26
Segment location on transcripts

| | Segment | |
|---|---|---|
| | starting | Segment ending |
| Transcript name | position | position |
| HUMF5A_PEA_1_T1 (SEQ ID NO: 33) | 2158 | 3766 |
| HUMF5A_PEA_1_T3 (SEQ ID NO: 34) | 2158 | 3766 |

Segment cluster HUMF5A_PEA_{—}1_node_{—}27 (SEQ ID NO:264) according to the present invention is supported by 12 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 27 below describes the starting and ending position of this segment on each transcript.

TABLE 27
Segment location on transcripts

| | Segment | |
|---|---|---|
| | starting | Segment ending |
| Transcript name | position | position |
| HUMF5A_PEA_1_T1 (SEQ ID NO: 33) | 3767 | 3936 |
| HUMF5A_PEA_1_T3 (SEQ ID NO: 34) | 3767 | 3936 |

Segment cluster HUMF5A_PEA_{—}1_node_{—}29 (SEQ ID NO:265) according to the present invention is supported by 22 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 28 below describes the starting and ending position of this segment on each transcript.

TABLE 28
Segment location on transcripts

| | Segment | |
|---|---|---|
| | starting | Segment ending |
| Transcript name | position | position |
| HUMF5A_PEA_1_T1 (SEQ ID NO: 33) | 3937 | 4978 |
| HUMF5A_PEA_1_T3 (SEQ ID NO: 34) | 3937 | 4978 |

Segment cluster HUMF5A_PEA_{—}1_node_{—}35 (SEQ ID NO:266) according to the present invention is supported by 7 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 29 below describes the starting and ending position of this segment on each transcript.

TABLE 29
Segment location on transcripts

| | Segment | |
|---|---|---|
| | starting | Segment ending |
| Transcript name | position | position |
| HUMF5A_PEA_1_T1 (SEQ ID NO: 33) | 5102 | 5338 |
| HUMF5A_PEA_1_T3 (SEQ ID NO: 34) | 5154 | 5390 |

Segment cluster HUMF5A_PEA_{—}1_node_{—}37 (SEQ ID NO:267) according to the present invention is supported by 9 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 30 below describes the starting and ending position of this segment on each transcript.

TABLE 30
Segment location on transcripts

| | | Segment |
|---|---|---|
| | Segment | ending |
| Transcript name | starting position | position |
| HUMF5A_PEA_1_T1 (SEQ ID NO: 33) | 5339 | 5549 |
| HUMF5A_PEA_1_T3 (SEQ ID NO: 34) | 5391 | 5601 |

Segment cluster HUMF5A_PEA_{—}1_node_{—}39 (SEQ ID NO:268) according to the present invention is supported by 10 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 31 below describes the starting and ending position of this segment on each transcript.

TABLE 31
Segment location on transcripts

| | | Segment |
|---|---|---|
| | Segment | ending |
| Transcript name | starting position | position |
| HUMF5A_PEA_1_T1 (SEQ ID NO: 33) | 5550 | 5729 |
| HUMF5A_PEA_1_T3 (SEQ ID NO: 34) | 5602 | 5781 |

Segment cluster HUMF5A_PEA_{—}1_node_{—}47 (SEQ ID NO:269) according to the present invention is supported by 14 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 32 below describes the starting and ending position of this segment on each transcript.

TABLE 32
Segment location on transcripts

| | | Segment |
|---|---|---|
| | Segment | ending |
| Transcript name | starting position | position |
| HUMF5A_PEA_1_T1 (SEQ ID NO: 33) | 6023 | 6178 |
| HUMF5A_PEA_1_T3 (SEQ ID NO: 34) | 6075 | 6230 |

Segment cluster HUMF5A_PEA_{—}1_node_{—}50 (SEQ ID NO:270) according to the present invention is supported by 20 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 33 below describes the starting and ending position of this segment on each transcript.

TABLE 33
Segment location on transcripts

| | | Segment |
|---|---|---|
| | Segment | ending |
| Transcript name | starting position | position |
| HUMF5A_PEA_1_T1 (SEQ ID NO: 33) | 6179 | 6316 |
| HUMF5A_PEA_1_T3 (SEQ ID NO: 34) | 6231 | 6368 |

Segment cluster HUMF5A_PEA_{—}1_node_{—}53 (SEQ ID NO:271) according to the present invention is supported by 29 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 34 below describes the starting and ending position of this segment on each transcript.

TABLE 34
Segment location on transcripts

| | | Segment |
|---|---|---|
| | Segment | ending |
| Transcript name | starting position | position |
| HUMF5A_PEA_1_T1 (SEQ ID NO: 33) | 6324 | 6475 |
| HUMF5A_PEA_1_T3 (SEQ ID NO: 34) | 6369 | 6520 |

Segment cluster HUMF5A_PEA_{—}1_node_{—}56 (SEQ ID NO:272) according to the present invention is supported by 24 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 35 below describes the starting and ending position of this segment on each transcript.

TABLE 35
Segment location on transcripts

| | | Segment |
|---|---|---|
| | Segment | ending |
| Transcript name | starting position | position |
| HUMF5A_PEA_1_T1 (SEQ ID NO: 33) | 6476 | 6611 |
| HUMF5A_PEA_1_T3 (SEQ ID NO: 34) | 6521 | 6656 |

Segment cluster HUMF5A_PEA_{—}1_node_{—}60 (SEQ ID NO:273) according to the present invention is supported by 24 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 36 below describes the starting and ending position of this segment on each transcript.

TABLE 36
Segment location on transcripts

| | | Segment |
|---|---|---|
| | Segment | ending |
| Transcript name | starting position | position |
| HUMF5A_PEA_1_T1 (SEQ ID NO: 33) | 6666 | 6951 |
| HUMF5A_PEA_1_T3 (SEQ ID NO: 34) | 6711 | 6996 |

According to an optional embodiment of the present invention, short segments related to the above cluster are also provided. These segments are up to about 120 bp in length, and so are included in a separate description.

Segment cluster HUMF5A_PEA_{—}1_node_{—}2 (SEQ ID NO:274) according to the present invention is supported by 6 libraries. The number of libraries was determined as previously described. This segment can be found in the following transcript(s): HUMF5A_PEA_{—}1_T1 (SEQ ID NO:33), HUMF5A_PEA_{—}1_T3 (SEQ ID NO:34) and HUMF5A_PEA_{—}1_T7 (SEQ ID NO:35). Table 37 below describes the starting and ending position of this segment on each transcript.

TABLE 37
Segment location on transcripts

| | | Segment |
|---|---|---|
| | Segment | ending |
| Transcript name | starting position | position |
| HUMF5A_PEA_1_T1 (SEQ ID NO: 33) | 341 | 432 |
| HUMF5A_PEA_1_T3 (SEQ ID NO: 34) | 341 | 432 |
| HUMF5A_PEA_1_T7 (SEQ ID NO: 35) | 341 | 432 |

Segment cluster HUMF5A_PEA_{—}1_node_{—}16 (SEQ ID NO:275) according to the present invention is supported by 10 libraries. The number of libraries was determined as previously described. This segment can be found in the following transcript(s): HUMF5A_PEA_{—}1_T1 (SEQ ID NO:33), HUMF5A_PEA_{—}1_T3 (SEQ ID NO:34) and HUMF5A_PEA_{—}1_T7 (SEQ ID NO:35). Table 38 below describes the starting and ending position of this segment on each transcript.

TABLE 38
Segment location on transcripts

| | | Segment |
|---|---|---|
| | Segment | ending |
| Transcript name | starting position | position |
| HUMF5A_PEA_1_T1 (SEQ ID NO: 33) | 1479 | 1578 |
| HUMF5A_PEA_1_T3 (SEQ ID NO: 34) | 1479 | 1578 |
| HUMF5A_PEA_1_T7 (SEQ ID NO: 35) | 1479 | 1578 |

Segment cluster HUMF5A_PEA_{—}1_node_{—}31 (SEQ ID NO:276) according to the present invention is supported by 3 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 39 below describes the starting and ending position of this segment on each transcript.

TABLE 39
Segment location on transcripts

| | | Segment |
|---|---|---|
| | Segment | ending |
| Transcript name | starting position | position |
| HUMF5A_PEA_1_T1 (SEQ ID NO: 33) | 4979 | 5033 |
| HUMF5A_PEA_1_T3 (SEQ ID NO: 34) | 4979 | 5033 |

Segment cluster HUMF5A_PEA_{—}1_node_{—}32 (SEQ ID NO:277) according to the present invention is supported by 2 libraries. The number of libraries was determined as previously described. This segment can be found in the following transcript(s): HUMF5A_PEA_{—}1_T3 (SEQ ID NO:34). Table 40 below describes the starting and ending position of this segment on each transcript.

TABLE 40
Segment location on transcripts

| | | Segment |
|---|---|---|
| | Segment | ending |
| Transcript name | starting position | position |
| HUMF5A_PEA_1_T3 (SEQ ID NO: 34) | 5034 | 5085 |

Segment cluster HUMF5A_PEA_{—}1_node_{—}33 (SEQ ID NO:278) according to the present invention is supported by 4 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 41 below describes the starting and ending position of this segment on each transcript.

TABLE 41
Segment location on transcripts

| | | Segment |
|---|---|---|
| | Segment | ending |
| Transcript name | starting position | position |
| HUMF5A_PEA_1_T1 (SEQ ID NO: 33) | 5034 | 5101 |
| HUMF5A_PEA_1_T3 (SEQ ID NO: 34) | 5086 | 5153 |

Segment cluster HUMF5A_PEA_{—}1_node_{—}41 (SEQ ID NO:279) according to the present invention is supported by 8 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 42 below describes the starting and ending position of this segment on each transcript.

TABLE 42
Segment location on transcripts

| | | Segment |
|---|---|---|
| | Segment | ending |
| Transcript name | starting position | position |
| HUMF5A_PEA_1_T1 (SEQ ID NO: 33) | 5730 | 5846 |
| HUMF5A_PEA_1_T3 (SEQ ID NO: 34) | 5782 | 5898 |

Segment cluster HUMF5A_PEA_{—}1_node_{—}43 (SEQ ID NO:280) according to the present invention is supported by 6 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 43 below describes the starting and ending position of this segment on each transcript.

TABLE 43
Segment location on transcripts

| | | Segment |
|---|---|---|
| | Segment | ending |
| Transcript name | starting position | position |
| HUMF5A_PEA_1_T1 (SEQ ID NO: 33) | 5847 | 5918 |
| HUMF5A_PEA_1_T3 (SEQ ID NO: 34) | 5899 | 5970 |

Segment cluster HUMF5A_PEA_{—}1_node_{—}45 (SEQ ID NO:281) according to the present invention is supported by 12 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 44 below describes the starting and ending position of this segment on each transcript.

TABLE 44
Segment location on transcripts

| | | Segment |
|---|---|---|
| | Segment | ending |
| Transcript name | starting position | position |
| HUMF5A_PEA_1_T1 (SEQ ID NO: 33) | 5919 | 6022 |
| HUMF5A_PEA_1_T3 (SEQ ID NO: 34) | 5971 | 6074 |

Segment cluster HUMF5A_PEA_{—}1_node_{—}51 (SEQ ID NO:282) according to the present invention can be found in the following transcript(s): HUMF5A_PEA_{—}1_T1 (SEQ ID NO:33). Table 45 below describes the starting and ending position of this segment on each transcript.

TABLE 45
Segment location on transcripts

| | Segment | Segment |
|---|---|---|
| Transcript name | starting position | ending position |
| HUMF5A_PEA_1_T1 | 6317 | 6323 |
| (SEQ ID NO: 33) | | |

Segment cluster HUMF5A_PEA_{—}1_node_{—}57 (SEQ ID NO:283) according to the present invention is supported by 18 libraries. The number of libraries was determined as previously described. This segment can be found in the following transcript(s): HUMF5A_PEA_{—}1_T1 (SEQ ID NO:33) and HUMF5A_PEA_{—}1_T3 (SEQ ID NO:34). Table 46 below describes the starting and ending position of this segment on each transcript.

TABLE 46
Segment location on transcripts

| | | Segment |
|---|---|---|
| | Segment | ending |
| Transcript name | starting position | position |
| HUMF5A_PEA_1_T1 (SEQ ID NO: 33) | 6612 | 6658 |
| HUMF5A_PEA_1_T3 (SEQ ID NO: 34) | 6657 | 6703 |

Segment cluster HUMF5A_PEA_{—}1_node_{—}59 (SEQ ID NO:284) according to the present invention can be found in the following transcript(s): HUMF5A_PEA_{—}1_T1 (SEQ ID NO:33) and HUMF5A_PEA_{—}1_T3 (SEQ ID NO:34). Table 47 below describes the starting and ending position of this segment on each transcript.

TABLE 47
Segment location on transcripts

| | | Segment |
|---|---|---|
| | Segment | ending |
| Transcript name | starting position | position |
| HUMF5A_PEA_1_T1 (SEQ ID NO: 33) | 6659 | 6665 |
| HUMF5A_PEA_1_T3 (SEQ ID NO: 34) | 6704 | 6710 |

### Variant Protein Alignment to the Previously Known Protein:

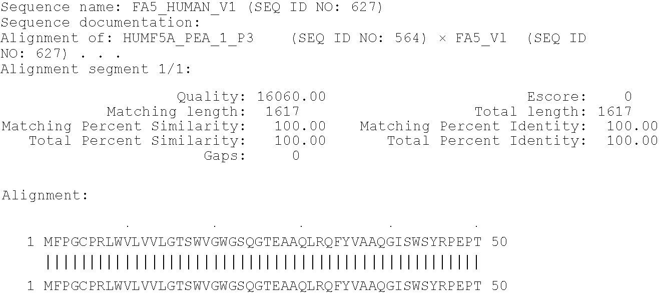

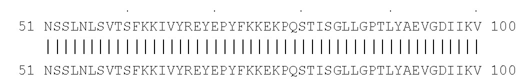

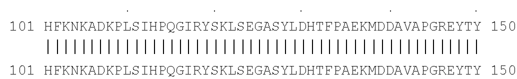

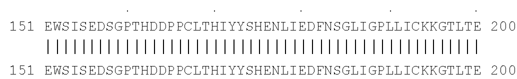

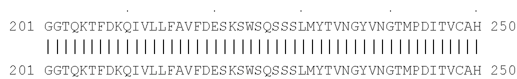

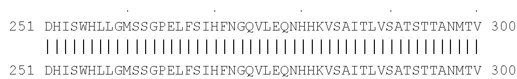

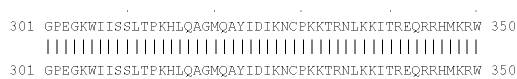

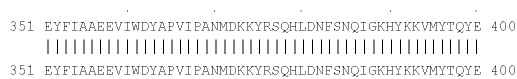

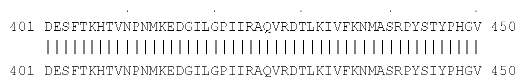

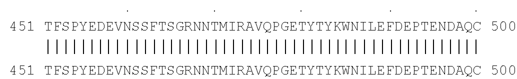

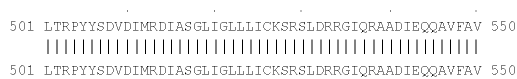

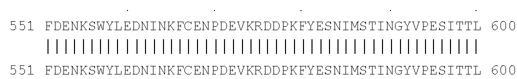

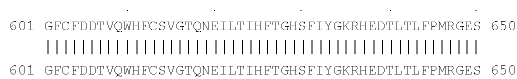

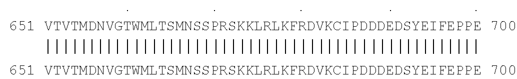

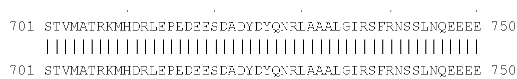

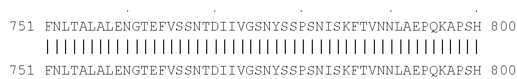

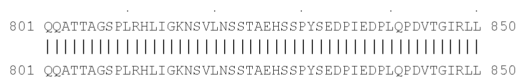

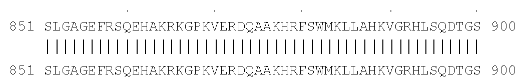

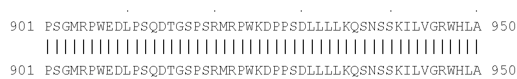

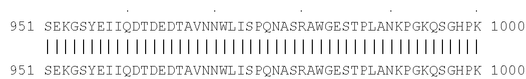

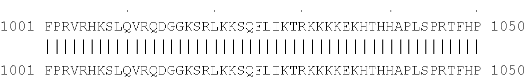

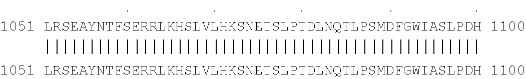

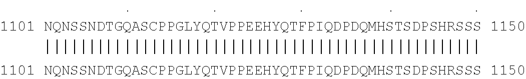

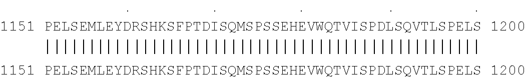

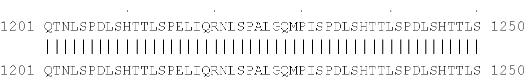

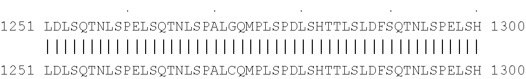

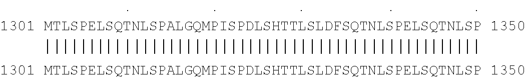

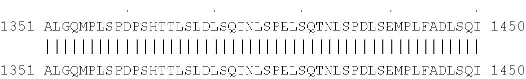

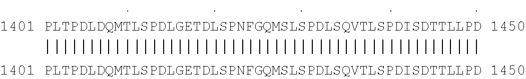

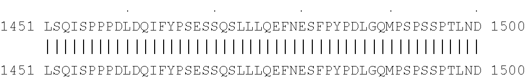

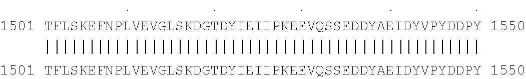

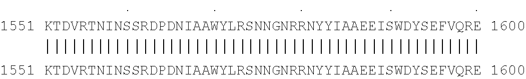

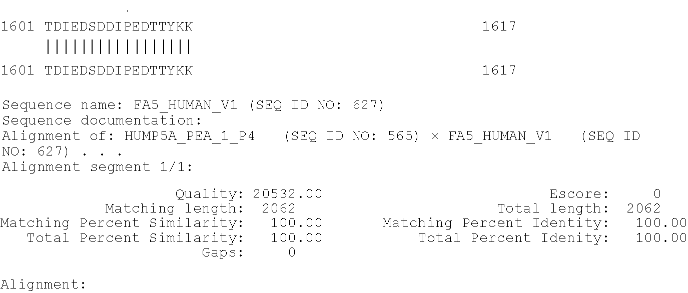

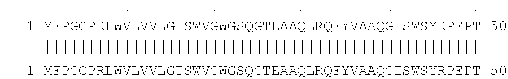

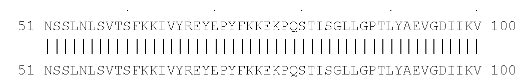

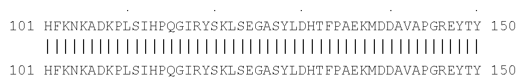

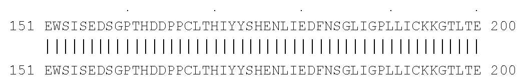

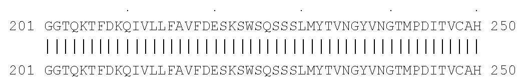

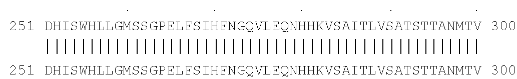

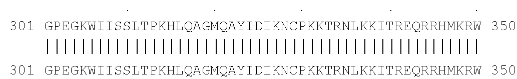

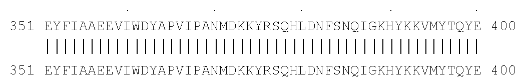

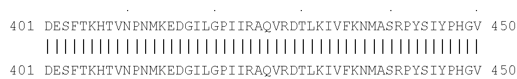

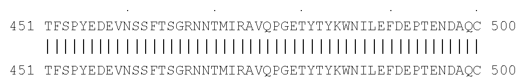

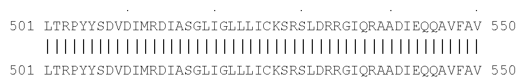

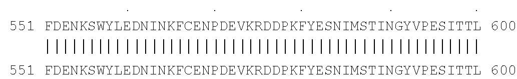

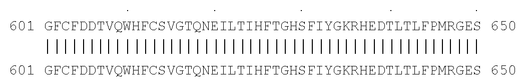

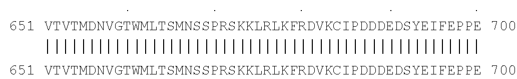

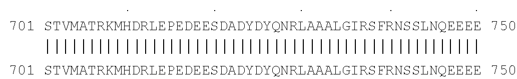

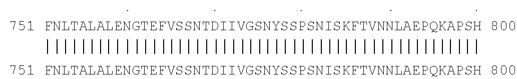

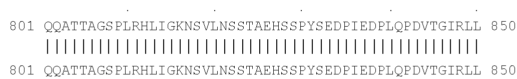

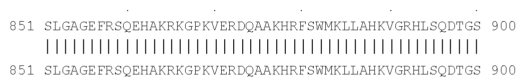

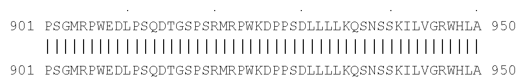

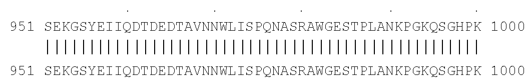

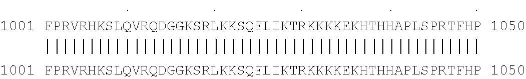

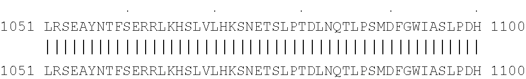

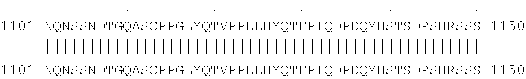

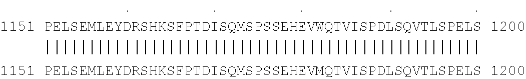

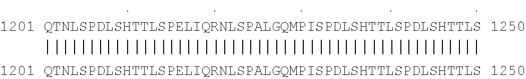

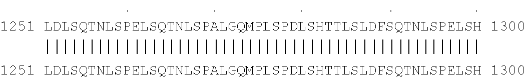

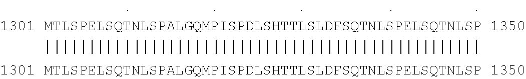

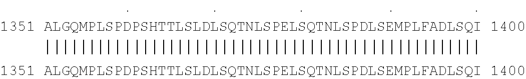

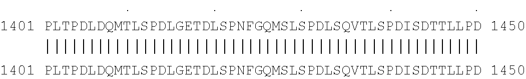

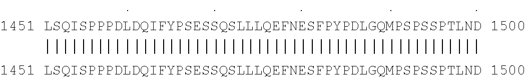

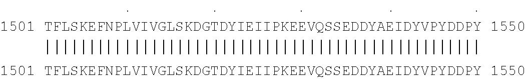

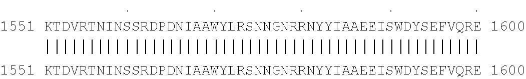

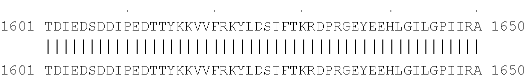

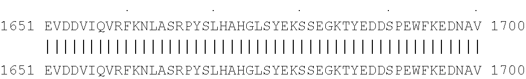

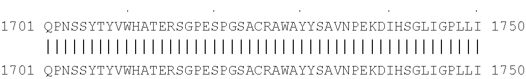

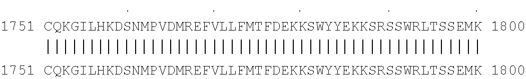

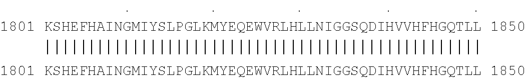

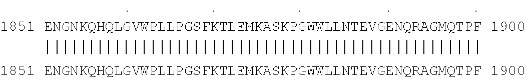

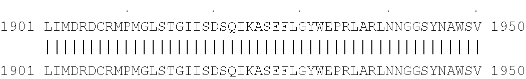

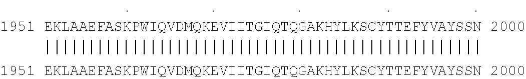

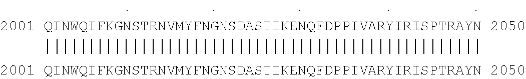

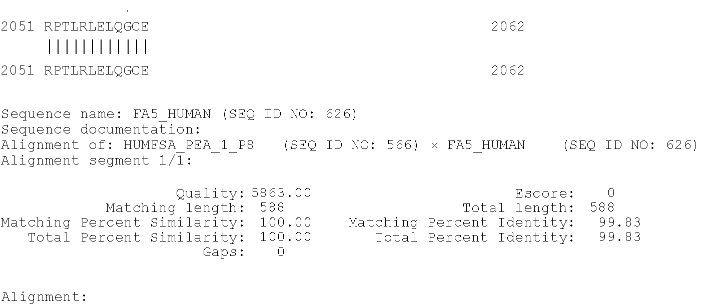

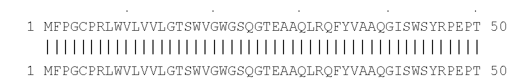

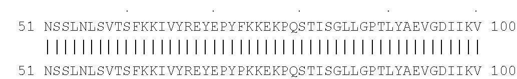

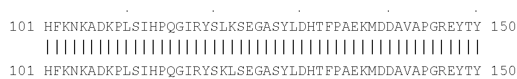

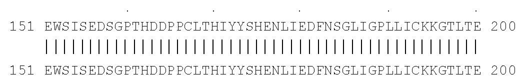

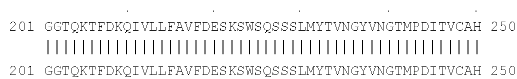

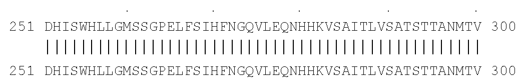

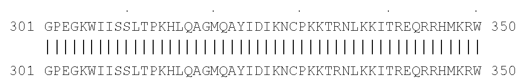

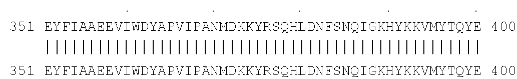

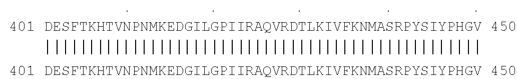

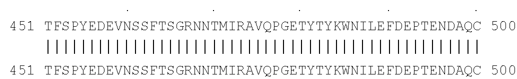

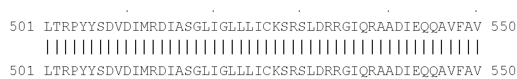

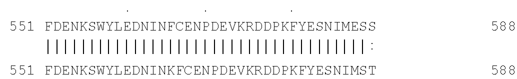

PBGD-amplicon, SEQ ID NO:531HPRT1-amplicon, SEQ ID NO:612HPRT1-amplicon, SEQ ID NO:615RPS27A amplicon, SEQ ID NO:1261

### Description for Cluster HUMANK

Cluster HUMANK features 8 transcript(s) and 22 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| HUMANK_T3 | 36 |
| HUMANK_T13 | 37 |
| HUMANK_T23 | 38 |
| HUMANK_T24 | 39 |
| HUMANK_T26 | 40 |
| HUMANK_T27 | 41 |
| HUMANK_T28 | 42 |
| HUMANK_T35 | 43 |

TABLE 2
Segments of interest

| Segment Name | SEQ ID NO: |
|---|---|
| HUMANK_node_91 | 285 |
| HUMANK_node_92 | 286 |
| HUMANK_node_93 | 287 |
| HUMANK_node_100 | 288 |
| HUMANK_node_108 | 289 |
| HUMANK_node_113 | 290 |
| HUMANK_node_115 | 291 |
| HUMANK_node_117 | 292 |
| HUMANK_node_119 | 293 |
| HUMANK_node_120 | 294 |
| HUMANK_node_94 | 295 |
| HUMANK_node_95 | 296 |
| HUMANK_node_98 | 297 |
| HUMANK_node_99 | 298 |
| HUMANK_node_102 | 299 |
| HUMANK_node_103 | 300 |
| HUMANK_node_104 | 301 |
| HUMANK_node_105 | 302 |
| HUMANK_node_106 | 303 |
| HUMANK_node_112 | 304 |
| HUMANK_node_114 | 305 |
| HUMANK_node_116 | 306 |

TABLE 3
Proteins of interest

| Protein Name | SEQ ID NO: | Corresponding Transcript(s) |
|---|---|---|
| HUMANK_P12 | 567 | HUMANK_T13 (SEQ ID NO: 37) |
| HUMANK_P21 | 568 | HUMANK_T26 (SEQ ID NO: 40) |
| HUMANK_P22 | 569 | HUMANK_T27 (SEQ ID NO: 41) |
| HUMANK_P23 | 570 | HUMANK_T28 (SEQ ID NO: 42) |
| HUMANK_P27 | 571 | HUMANK_T35 (SEQ ID NO: 43) |
| HUMANK_P29 | 572 | HUMANK_T3 (SEQ ID NO: 36) |
| HUMANK_P33 | 573 | HUMANK_T23 (SEQ ID NO: 38) |
| HUMANK_P34 | 574 | HUMANK_T24 (SEQ ID NO: 39) |

These sequences are variants of the known protein Ankyrin 1 (SwissProt accession identifier ANK1_HUMAN; known also according to the synonyms Erythrocyte ankyrin; Ankyrin R), SEQ ID NO: 628, referred to herein as the previously known protein.

Protein Ankyrin 1 (SEQ ID NO:628) is known or believed to have the following function(s): Attach integral membrane proteins to cytoskeletal elements; bind to the erythrocyte membrane protein band 4.2, to Na—K ATPase, to the lymphocyte membrane protein GP85, and to the cytoskeletal proteins fodrin, tubulin, vimentin and desmin. Erythrocyte ankyrins also link spectrin (beta chain) to the cytoplasmic domain of the erythrocytes anion exchange protein; they retain most or all of these binding functions. The sequence for protein Ankyrin 1 is given at the end of the application, as “Ankyrin 1 amino acid sequence”. Known polymorphisms for this sequence are as shown in Table 4.

TABLE 4
Amino acid mutations for Known Protein

| SNP position(s) | |
|---|---|
| on amino | |
| acid sequence | Comment |
| 20 | R -> T. /FTId = VAR_000595. |
| 462 | V -> I (in HS). /FTId = VAR_000596. |
| 618 | R -> H (in Brueggen). /FTId = VAR_000597. |
| 749 | V -> A. /FTId = VAR_000598. |
| 844 | D -> E. /FTId = VAR_000599. |
| 1285 | E -> D. /FTId = VAR_000601. |
| 1391 | S -> T. /FTId = VAR_000600. |
| 1591 | D -> N (in Duesseldorf). /FTId = VAR_000602. |
| 1698 | R -> D. /FTId = VAR_000603. |
| 229 | A -> S |
| 1545 | V -> I |

Protein Ankyrin 1 (SEQ ID NO:628) localization is believed to be Cytoplasmic surface of erythrocytic plasma membrane.

The following GO Annotation(s) apply to the previously known protein. The following annotation(s) were found: exocytosis; cytoskeleton organization and biogenesis; signal transduction, which are annotation(s) related to Biological Process; structural protein; structural protein of cytoskeleton; cytoskeletal adaptor, which are annotation(s) related to Molecular Function; and cytoskeleton; plasma membrane; actin cytoskeleton; basolateral plasma membrane, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster HUMANK can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 23 and Table 5. This cluster is overexpressed (at least at a minimum level) in the following pathological conditions: epithelial malignant tumors.

TABLE 5
Normal tissue distribution

| Name of Tissue | Number |
|---|---|
| Bladder | 0 |
| Brain | 41 |
| Epithelial | 2 |
| General | 20 |
| head and neck | 0 |
| Kidney | 0 |
| bone marrow | 62 |
| Muscle | 225 |
| Ovary | 0 |
| Pancreas | 4 |
| prostate | 8 |
| uterus | 0 |

TABLE 6
P values and ratios for expression in cancerous tissue

| Name of Tissue | P1 | P2 | SP1 | R3 | SP2 | R4 |
|---|---|---|---|---|---|---|
| bladder | 5.4e−01 | 6.0e−01 | 5.6e−01 | 1.8 | 6.8e−01 | 1.5 |
| brain | 8.9e−01 | 8.9e−01 | 1 | 0.1 | 1 | 0.2 |
| epithelial | 1.0e−01 | 1.7e−01 | 1.8e−03 | 3.8 | 1.8e−02 | 2.6 |
| general | 9.1e−01 | 9.5e−01 | 9.3e−01 | 0.5 | 1 | 0.4 |
| head and neck | 4.3e−01 | 2.8e−01 | 1 | 1.1 | 7.5e−01 | 1.4 |
| kidney | 6.5e−01 | 7.2e−01 | 5.8e−01 | 1.7 | 7.0e−01 | 1.4 |
| bone marrow | 7.1e−01 | 8.4e−01 | 1 | 0.3 | 9.0e−01 | 0.6 |
| muscle | 5.4e−01 | 6.2e−01 | 1 | 0.1 | 1 | 0.2 |
| ovary | 3.8e−01 | 4.2e−01 | 6.9e−02 | 2.4 | 1.6e−01 | 1.9 |
| pancreas | 5.5e−01 | 2.0e−01 | 4.2e−01 | 1.7 | 1.5e−01 | 2.6 |
| prostate | 9.1e−01 | 9.3e−01 | 6.7e−01 | 1.1 | 7.5e−01 | 1.0 |
| uterus | 4.7e−01 | 6.4e−01 | 6.6e−01 | 1.5 | 8.0e−01 | 1.2 |

As noted above, cluster HUMANK features 8 transcript(s), which were listed in Table 1 above. These transcript(s) encode for protein(s) which are variant(s) of protein Ankyrin 1 (SEQ ID NO:628). A description of each variant protein according to the present invention is now provided.

Variant protein HUMANK_P12 (SEQ ID NO:567) according to the present invention has an amino acid sequence as given at the end of the application; it is encoded by transcript(s) HUMANK_T13 (SEQ ID NO:37). An alignment is given to the known protein (Ankyrin 1 (SEQ ID NO:628)) at the end of the application. One or more alignments to one or more previously published protein sequences are given at the end of the application. A brief description of the relationship of the variant protein according to the present invention to each such aligned protein is as follows:

Comparison Report Between HUMANK_P12 (SEQ ID NO:567) and AAH07930 (SEQ ID NO 631):

1. An isolated chimeric polypeptide encoding for HUMANK_P12 (SEQ ID NO:567), comprising a first amino acid sequence being at least 90% homologous to MWTFVTQLLVTLVLLSFFLVSCQNVMHIVRGSLCFVLKHIHQELDKELGESEGLSDDEETISTRVVRRRVF LKGNEFQNIPGEQVTEEQFTDEQGNIVTKKIIRKVVRQIDLSSADAAQEHEE corresponding to amino acids 1-123 of AAH07930 (SEQ ID NO:631), which also corresponds to amino acids 1-123 of HUMANK_P12 (SEQ ID NO:567), and a second amino acid sequence being at least 70%, optionally at least 80%, preferably at least 85%, more preferably at least 90% and most preferably at least 95% homologous to a polypeptide having the sequence VTVEGPLEDPSELEVDIDYFMKHSKDHTSTPNP (SEQ ID NO:1509) corresponding to amino acids 124-156 of HUMANK_P12 (SEQ ID NO:567), wherein said first amino acid sequence and second amino acid sequence are contiguous and in a sequential order.

2. An isolated polypeptide encoding for a tail of HUMANK_P12 (SEQ ID NO:567), comprising a polypeptide being at least 70%, optionally at least about 80%, preferably at least about 85%, more preferably at least about 90% and most preferably at least about 95% homologous to the sequence VTVEGPLEDPSELEVDIDYFMKHSKDHTSTPNP (SEQ ID NO:1509) in HUMANK_P12 (SEQ ID NO:567).

Comparison Report Between HUMANK_P12 (SEQ ID NO:567) and ANK1_HUMAN_V1 (SEQ ID NO 629):

1. An isolated chimeric polypeptide encoding for HUMANK_P12 (SEQ ID NO:567), comprising a first amino acid sequence being at least 70%, optionally at least 80%, preferably at least 85%, more preferably at least 90% and most preferably at least 95% homologous to a polypeptide having the sequence MWTFVTQLLVTLVLLSFFLVSCQNVMHIVRGSLCFVLKHIHQELDKELGESEGLSDDEETISTRVVRRRVF LK (SEQ ID NO:1510) corresponding to amino acids 1-73 of HUMANK_P12 (SEQ ID NO:567), and a second amino acid sequence being at least 90% homologous to GNEFQNIPGEQVTEEQFTDEQGNIVTKKIIRKVVRQIDLSSADAAQEHEEVTVEGPLEDPSELEVDIDYFMK HSKDHTSTPNP (SEQ ID NO:1509) corresponding to amino acids 1799-1881 of ANK1_HUMAN_V1 (SEQ ID NO:629), which also corresponds to amino acids 74-156 of HUMANK_P12 (SEQ ID NO:567), wherein said first amino acid sequence and second amino acid sequence are contiguous and in a sequential order.

2. An isolated polypeptide encoding for a head of HUMANK_P12 (SEQ ID NO:567), comprising a polypeptide being at least 70%, optionally at least about 80%, preferably at least about 85%, more preferably at least about 90% and most preferably at least about 95% homologous to the sequence MWTFVTQLLVTLVLLSFFLVSCQNVMHIVRGSLCFVLKHIHQELDKELGESEGLSDDEETISTRVVRRRVF LK (SEQ ID NO:1510) of HUMANK_P12 (SEQ ID NO:567).

It should be noted that the known protein sequence (ANK1_HUMAN (SEQ ID NO:628)) has one or more changes than the sequence given at the end of the application and named as being the amino acid sequence for ANK1_HUMAN_V1 (SEQ ID NO:629). These changes were previously known to occur and are listed in the table below.

TABLE 7
Changes to ANK1_HUMAN_V1 (SEQ ID NO: 629)

| SNP position(s) on amino | |
|---|---|
| acid sequence | Type of change |
| 1 | init_met |

Comparison Report Between HUMANK_P12 (SEQ ID NO:567) and Q8N604 (SEQ ID NO: 630):

1. An isolated chimeric polypeptide encoding for HUMANK_P12 (SEQ ID NO:567), comprising a first amino acid sequence being at least 90% homologous to MWTFVTQLLVTLVLLSFFLVSCQNVMHIVRGSLCFVLKHIHQELDKELGESE corresponding to amino acids 1-52 of Q8N604 (SEQ ID NO:630), which also corresponds to amino acids 1-52 of HUMANK_P12 (SEQ ID NO:567), a bridging amino acid G corresponding to amino acid 53 of HUMANK_P12 (SEQ ID NO:567), a second amino acid sequence being at least 90% homologous to LSDDEETISTRVVRRRVFLKGNEFQNIPGEQVTEEQFTDEQGNIVTKKIIRKVVRQIDLSSADAAQEHEEV corresponding to amino acids 54-124 of Q8N604 (SEQ ID NO:630), which also corresponds to amino acids 54-124 of HUMANK_P12 (SEQ ID NO:567), and a third amino acid sequence being at least 70%, optionally at least 80%, preferably at least 85%, more preferably at least 90% and most preferably at least 95% homologous to a polypeptide having the sequence TVEGPLEDPSELEVDIDYFMKHSKDHTSTPNP corresponding to amino acids 125-156 of HUMANK_P12 (SEQ ID NO:567), wherein said first amino acid sequence, bridging amino acid, second amino acid sequence and third amino acid sequence are contiguous and in a sequential order.

2. An isolated polypeptide encoding for a tail of HUMANK_P12 (SEQ ID NO:567), comprising a polypeptide being at least 70%, optionally at least about 80%, preferably at least about 85%, more preferably at least about 90% and most preferably at least about 95% homologous to the sequence TVEGPLEDPSELEVDIDYFMKHSKDHTSTPNP in HUMANK_P12 (SEQ ID NO:567).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMANK_P12 (SEQ ID NO:567) also has the following non-silent SNPs (Single Nucleotide Polymorphisms) as listed in Table 8, (given according to their position(s) on the amino acid sequence, with the alternative amino acid(s) listed; the last column indicates whether the SNP is known or not; the presence of known SNPs in variant protein HUMANK_P12 (SEQ ID NO:567) sequence provides support for the deduced sequence of this variant protein according to the present invention).

TABLE 8
Amino acid mutations

| SNP position(s) on amino acid | Alternative | Previously |
|---|---|---|
| sequence | amino acid(s) | known SNP? |
| 12 | L -> P | No |
| 63 | T -> P | No |
| 82 | G -> | No |
| 82 | G -> V | No |
| 89 | Q -> P | No |

Variant protein HUMANK_P12 (SEQ ID NO:567) is encoded by the following transcript(s): HUMANK_T13 (SEQ ID NO:37), for which the sequence(s) is/are given at the end of the application. The coding portion of transcript HUMANK_T13 (SEQ ID NO:37) is shown in bold; this coding portion starts at position 2053 and ends at position 2520. The transcript also has the following SNPs as listed in Table 9 (given according to their position on the nucleotide sequence, with the alternative nucleic acid listed; the last column indicates whether the SNP is known or not; the presence of known SNPs in variant protein HUMANK_P12 (SEQ ID NO:567) sequence provides support for the deduced sequence of this variant protein according to the present invention).

TABLE 9
Nucleic acid SNPs

| SNP position on nucleotide | Alternative | |
|---|---|---|
| sequence | nucleic acid | Previously known SNP? |
| 1603 | C -> T | No |
| 1969 | A -> G | No |
| 2005 | -> G | No |
| 2014 | C -> A | No |
| 2087 | T -> C | No |
| 2238 | C -> A | No |
| 2239 | A -> C | No |
| 2297 | G -> | No |
| 2297 | G -> T | No |
| 2318 | A -> C | No |
| 2533 | -> C | No |
| 2533 | -> T | No |
| 2733 | A -> | No |
| 2733 | A -> C | No |
| 2742 | A -> C | No |
| 2772 | A -> C | No |
| 2796 | A -> | No |
| 2796 | A -> C | No |
| 2890 | C -> T | No |
| 3305 | -> G | No |
| 3306 | -> C | No |
| 4118 | T -> A | Yes |
| 4175 | -> T | No |
| 4228 | G -> | No |
| 4603 | G -> T | Yes |
| 5012 | T -> | No |

Variant protein HUMANK_P21 (SEQ ID NO:568) according to the present invention has an amino acid sequence as given at the end of the application; it is encoded by transcript(s) HUMANK_T26 (SEQ ID NO:40). An alignment is given to the known protein (Ankyrin 1 (SEQ ID NO:628)) at the end of the application. One or more alignments to one or more previously published protein sequences are given at the end of the application. A brief description of the relationship of the variant protein according to the present invention to each such aligned protein is as follows:

Comparison Report Between HUMANK_P21 (SEQ ID NO:568) and AAH07930 (SEQ ID NO: 631):

1. An isolated chimeric polypeptide encoding for HUMANK_P21 (SEQ ID NO:568), comprising a first amino acid sequence being at least 90% homologous to MWTFVTQLLVTLVLLSFFLVSCQNVMHIVRGSLCFVLKHIHQELDKELGESEGLSDDEETISTRVVRRRVF LKGNEFQNIPGEQVTEEQFTDEQGNIVTKKIIRKVVRQIDLSSADAAQEHEE corresponding to amino acids 1-123 of AAH07930 (SEQ ID NO:631), which also corresponds to amino acids 1-123 of HUMANK_P21 (SEQ ID NO:568), and a second amino acid sequence being at least 70%, optionally at least 80%, preferably at least 85%, more preferably at least 90% and most preferably at least 95% homologous to a polypeptide having the sequence VTVEGPLEDPSELEVELRGSGLQPDLIEGRKGAQIVKRASLKRGKQ (SEQ ID NO:1511) corresponding to amino acids 124-169 of HUMANK_P21 (SEQ ID NO:568), wherein said first amino acid sequence and second amino acid sequence are contiguous and in a sequential order.

2. An isolated polypeptide encoding for a tail of HUMANK_P21 (SEQ ID NO:568), comprising a polypeptide being at least 70%, optionally at least about 80%, preferably at least about 85%, more preferably at least about 90% and most preferably at least about 95% homologous to the sequence VTVEGPLEDPSELEVELRGSGLQPDLIEGRKGAQIVKRASLKRGKQ (SEQ ID NO:1511) in HUMANK_P21 (SEQ ID NO:568).

Comparison Report Between HUMANK_P21 (SEQ ID NO:568) and Q8N604 (SEQ ID NO:630):

1. An isolated chimeric polypeptide encoding for HUMANK_P21 (SEQ ID NO:568), comprising a first amino acid sequence being at least 90% homologous to MWTFVTQLLVTLVLLSFFLVSCQNVMHIVRGSLCFVLKHIHQELDKELGESE corresponding to amino acids 1-52 of Q8N604 (SEQ ID NO:630), which also corresponds to amino acids 1-52 of HUMANK_P21 (SEQ ID NO:568), a bridging amino acid G corresponding to amino acid 53 of HUMANK_P21 (SEQ ID NO:568), a second amino acid sequence being at least 90% homologous to LSDDEETISTRVVRRRVFLKGNEFQNIPGEQVTEEQFTDEQGNIVTKKIIRKVVRQIDLSSADAAQEHE corresponding to amino acids 54-122 of Q8N604 (SEQ ID NO:630), which also corresponds to amino acids 54-122 of HUMANK_P21 (SEQ ID NO:568), a third amino acid sequence being at least 70%, optionally at least 80%, preferably at least 85%, more preferably at least 90% and most preferably at least 95% homologous to a polypeptide having the sequence EVTVEGPLEDPSEL (SEQ ID NO:1512) corresponding to amino acids 123-136 of HUMANK_P21 (SEQ ID NO:568), and a fourth amino acid sequence being at least 90% homologous to EVELRGSGLQPDLIEGRKGAQIVKRASLKRGKQ corresponding to amino acids 123-155 of Q8N604 (SEQ ID NO:630), which also corresponds to amino acids 137-169 of HUMANK_P21 (SEQ ID NO:568), wherein said first amino acid sequence, bridging amino acid, second amino acid sequence, third amino acid sequence and fourth amino acid sequence are contiguous and in a sequential order.

2. An isolated polypeptide encoding for an edge portion of HUMANK_P21 (SEQ ID NO:568), comprising an amino acid sequence being at least 70%, optionally at least about 80%, preferably at least about 85%, more preferably at least about 90% and most preferably at least about 95% homologous to the sequence encoding for EVTVEGPLEDPSEL (SEQ ID NO:1512), corresponding to HUMANK_P21 (SEQ ID NO:568).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMANK_P21 (SEQ ID NO:568) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein HUMANK_P21 (SEQ ID NO:568) sequence provides support for the deduced sequence of this variant protein according to the present invention).

TABLE 10
Amino acid mutations

| SNP position(s) on amino acid | Alternative | Previously |
|---|---|---|
| sequence | amino acid(s) | known SNP? |
| 12 | L -> P | No |
| 63 | T -> P | No |
| 82 | G -> | No |
| 82 | G -> V | No |
| 89 | Q -> P | No |

Variant protein HUMANK_P21 (SEQ ID NO:568) is encoded by the following transcript(s): HUMANK_T26 (SEQ ID NO:40), for which the sequence(s) is/are given at the end of the application. The coding portion of transcript HUMANK_T26 (SEQ ID NO:40) is shown in bold; this coding portion starts at position 2053 and ends at position 2559. The transcript also has the following SNPs as listed in Table 11 (given according to their position on the nucleotide sequence, with the alternative nucleic acid listed; the last column indicates whether the SNP is known or not; the presence of known SNPs in variant protein HUMANK_P21 (SEQ ID NO:568) sequence provides support for the deduced sequence of this variant protein according to the present invention).

TABLE 11
Nucleic acid SNPs

| SNP position on nucleotide | Alternative | Previously |
|---|---|---|
| sequence | nucleic acid | known SNP? |
| 1603 | C -> T | No |
| 1969 | A -> G | No |
| 2005 | -> G | No |
| 2014 | C -> A | No |
| 2087 | T -> C | No |
| 2238 | C -> A | No |
| 2239 | A -> C | No |
| 2297 | G -> | No |
| 2297 | G -> T | No |
| 2318 | A -> C | No |
| 2635 | -> C | No |
| 2635 | -> T | No |
| 2835 | A -> | No |
| 2835 | A -> C | No |
| 2844 | A -> C | No |
| 2874 | A -> C | No |
| 2898 | A -> | No |
| 2898 | A -> C | No |
| 2992 | C -> T | No |
| 3407 | -> G | No |
| 3408 | -> C | No |
| 4220 | T -> A | Yes |
| 4277 | -> T | No |
| 4330 | G -> | No |
| 4705 | G -> T | Yes |
| 5114 | T -> | No |

Variant protein HUMANK_P22 (SEQ ID NO:569) according to the present invention has an amino acid sequence as given at the end of the application; it is encoded by transcript(s) HUMANK_T27 (SEQ ID NO:41). An alignment is given to the known protein (Ankyrin 1 (SEQ ID NO:628)) at the end of the application. One or more alignments to one or more previously published protein sequences are given at the end of the application. A brief description of the relationship of the variant protein according to the present invention to each such aligned protein is as follows:

Comparison Report Between HUMANK_P22 (SEQ ID NO:569) and AAH07930 (SEQ ID NO:631):

1. An isolated chimeric polypeptide encoding for HUMANK_P22 (SEQ ID NO:569), comprising a first amino acid sequence being at least 90% homologous to MWTFVTQLLVTLVLLSFFLVSCQNVMHIVRGSLCFVLKHIHQELDKELGESEGLSDDEETISTRVVRRRVF LKGNEFQNIPGEQVTEEQFTDEQGNIVTKKIIRKVVRQIDLSSADAAQEHEE corresponding to amino acids 1-123 of AAH07930 (SEQ ID NO:631), which also corresponds to amino acids 1-123 of HUMANK_P22 (SEQ ID NO:569), and a second amino acid sequence being at least 70%, optionally at least 80%, preferably at least 85%, more preferably at least 90% and most preferably at least 95% homologous to a polypeptide having the sequence VTVEGPLEDPSELEVDIDYFMKHSKVELRGSGLQPDLIEGRKGAQIVKRASLKRGKQ (SEQ ID NO:1513) corresponding to amino acids 124-180 of HUMANK_P22 (SEQ ID NO:569), wherein said first amino acid sequence and second amino acid sequence are contiguous and in a sequential order.

2. An isolated polypeptide encoding for a tail of HUMANK_P22 (SEQ ID NO:569), comprising a polypeptide being at least 70%, optionally at least about 80%, preferably at least about 85%, more preferably at least about 90% and most preferably at least about 95% homologous to the sequence VTVEGPLEDPSELEVDIDYFMKHSKVELRGSGLQPDLIEGRKGAQIVKRASLKRGKQ (SEQ ID NO:1513) in HUMANK_P22 (SEQ ID NO:569).

Comparison Report Between HUMANK_P22 (SEQ ID NO:569) and Q8N604 (SEQ ID NO:630):

1. An isolated chimeric polypeptide encoding for HUMANK_P22 (SEQ ID NO:569), comprising a first amino acid sequence being at least 90% homologous to MWTFVTQLLVTLVLLSFFLVSCQNVMHIVRGSLCFVLKHIHQELDKELGESE corresponding to amino acids 1-52 of Q8N604 (SEQ ID NO:630), which also corresponds to amino acids 1-52 of HUMANK_P22 (SEQ ID NO:569), a bridging amino acid G corresponding to amino acid 53 of HUMANK_P22 (SEQ ID NO:569), a second amino acid sequence being at least 90% homologous to LSDDEETISTRVVRRRVFLKGNEFQNIPGEQVTEEQFTDEQGNIVTKKIIRKVVRQIDLSSADAAQEHEE corresponding to amino acids 54-123 of Q8N604 (SEQ ID NO:630), which also corresponds to amino acids 54-123 of HUMANK_P22 (SEQ ID NO:569), a third amino acid sequence being at least 70%, optionally at least 80%, preferably at least 85%, more preferably at least 90% and most preferably at least 95% homologous to a polypeptide having the sequence VTVEGPLEDPSELEVDIDYFMKHSK (SEQ ID NO:1514) corresponding to amino acids 124-148 of HUMANK_P22 (SEQ ID NO:569), and a fourth amino acid sequence being at least 90% homologous to VELRGSGLQPDLIEGRKGAQIVKRASLKRGKQ (SEQ ID NO:1517) corresponding to amino acids 124-155 of Q8N604 (SEQ ID NO:630), which also corresponds to amino acids 149-180 of HUMANK_P22 (SEQ ID NO:569), wherein said first amino acid sequence, bridging amino acid, second amino acid sequence, third amino acid sequence and fourth amino acid sequence are contiguous and in a sequential order.

2. An isolated polypeptide encoding for an edge portion of HUMANK_P22 (SEQ ID NO:569), comprising an amino acid sequence being at least 70%, optionally at least about 80%, preferably at least about 85%, more preferably at least about 90% and most preferably at least about 95% homologous to the sequence encoding for VTVEGPLEDPSELEVDIDYFMKHSK (SEQ ID NO:1514), corresponding to HUMANK_P22 (SEQ ID NO:569).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMANK_P22 (SEQ ID NO:569) also has the following non-silent SNPs (Single Nucleotide Polymorphisms) as listed in Table 12, (given according to their position(s) on the amino acid sequence, with the alternative amino acid(s) listed; the last column indicates whether the SNP is known or not; the presence of known SNPs in variant protein HUMANK_P22 (SEQ ID NO:569) sequence provides support for the deduced sequence of this variant protein according to the present invention).

TABLE 12
Amino acid mutations

| SNP position(s) on amino acid | Alternative | Previously |
|---|---|---|
| sequence | amino acid(s) | known SNP? |
| 12 | L -> P | No |
| 63 | T -> P | No |
| 82 | G -> | No |
| 82 | G -> V | No |
| 89 | Q -> P | No |

Variant protein HUMANK_P22 (SEQ ID NO:569) is encoded by the following transcript(s): HUMANK_T27 (SEQ ID NO:41), for which the sequence(s) is/are given at the end of the application. The coding portion of transcript HUMANK_T27 (SEQ ID NO:41) is shown in bold; this coding portion starts at position 2053 and ends at position 2592. The transcript also has the following SNPs as listed in Table 13 (given according to their position on the nucleotide sequence, with the alternative nucleic acid listed; the last column indicates whether the SNP is known or not; the presence of known SNPs in variant protein HUMANK_P22 (SEQ ID NO:569) sequence provides support for the deduced sequence of this variant protein according to the present invention).

TABLE 13
Nucleic acid SNPs

| SNP position on nucleotide | Alternative | Previously |
|---|---|---|
| sequence | nucleic acid | known SNP? |
| 1603 | C -> T | No |
| 1969 | A -> G | No |
| 2005 | -> G | No |
| 2014 | C -> A | No |
| 2087 | T -> C | No |
| 2238 | C -> A | No |
| 2239 | A -> C | No |
| 2297 | G -> | No |
| 2297 | G -> T | No |
| 2318 | A -> C | No |
| 2668 | -> C | No |
| 2668 | -> T | No |
| 2868 | A -> | No |
| 2868 | A -> C | No |
| 2877 | A -> C | No |
| 2907 | A -> C | No |
| 2931 | A -> | No |
| 2931 | A -> C | No |
| 3025 | C -> T | No |
| 3440 | -> G | No |
| 3441 | -> C | No |
| 4253 | T -> A | Yes |
| 4310 | -> T | No |
| 4363 | G -> | No |
| 4738 | G -> T | Yes |
| 5147 | T -> | No |

Variant protein HUMANK_P23 (SEQ ID NO:570) according to the present invention has an amino acid sequence as given at the end of the application; it is encoded by transcript(s) HUMANK_T28 (SEQ ID NO:42). An alignment is given to the known protein (Ankyrin 1 (SEQ ID NO:628)) at the end of the application. One or more alignments to one or more previously published protein sequences are given at the end of the application. A brief description of the relationship of the variant protein according to the present invention to each such aligned protein is as follows:

Comparison Report Between HUMANK_P23 (SEQ ID NO:570) and AAH07930 (SEQ ID NO:631):

1. An isolated chimeric polypeptide encoding for HUMANK_P23 (SEQ ID NO:570), comprising a first amino acid sequence being at least 90% homologous to MWTFVTQLLVTLVLLSFFLVSCQNVMHIVRGSLCFVLKHIHQELDKELGESEGLSDDEETISTRVVRRRVF LKGNEFQNIPGEQVTEEQFTDEQGNUVTKKIIRKVVRQIDLSSADAAQEHEE corresponding to amino acids 1-123 of AAH07930 (SEQ ID NO:631), which also corresponds to amino acids 1-123 of HUMANK_P23 (SEQ ID NO:570), and a second amino acid sequence being at least 70%, optionally at least 80%, preferably at least 85%, more preferably at least 90% and most preferably at least 95% homologous to a polypeptide having the sequence VTVEGPLEDPSELEDHTSTPNP (SEQ ID NO:1515) corresponding to amino acids 124-145 of HUMANK_P23 (SEQ ID NO:570), wherein said first amino acid sequence and second amino acid sequence are contiguous and in a sequential order.

2. An isolated polypeptide encoding for a tail of HUMANK_P23 (SEQ ID NO:570), comprising a polypeptide being at least 70%, optionally at least about 80%, preferably at least about 85%, more preferably at least about 90% and most preferably at least about 95% homologous to the sequence VTVEGPLEDPSELEDHTSTPNP (SEQ ID NO:1515) in HUMANK_P23 (SEQ ID NO:570).

Comparison Report Between HUMANK_P23 (SEQ ID NO:570) and Q8N604 (SEQ ID NO:630):

1. An isolated chimeric polypeptide encoding for HUMANK_P23 (SEQ ID NO:570), comprising a first amino acid sequence being at least 90% homologous to MWTFVTQLLVTLVLLSFFLVSCQNVMHIVRGSLCFVLKHIHQELDKELGESE corresponding to amino acids 1-52 of Q8N604 (SEQ ID NO:630), which also corresponds to amino acids 1-52 of HUMANK_P23 (SEQ ID NO:570), a bridging amino acid G corresponding to amino acid 53 of HUMANK_P23 (SEQ ID NO:570), a second amino acid sequence being at least 90% homologous to LSDDEETISTRVVRRRVFLKGNEFQNIPGEQVTEEQFTDEQGNIVTKKIIRKVVRQIDLSSADAAQEHEEV corresponding to amino acids 54-124 of Q8N604 (SEQ ID NO:630), which also corresponds to amino acids 54-124 of HUMANK_P23 (SEQ ID NO:570), and a third amino acid sequence being at least 70%, optionally at least 80%, preferably at least 85%, more preferably at least 90% and most preferably at least 95% homologous to a polypeptide having the sequence TVEGPLEDPSELEDHTSTPNP corresponding to amino acids 125-145 of HUMANK_P23 (SEQ ID NO:570), wherein said first amino acid sequence, bridging amino acid, second amino acid sequence and third amino acid sequence are contiguous and in a sequential order.

2. An isolated polypeptide encoding for a tail of HUMANK_P23 (SEQ ID NO:570), comprising a polypeptide being at least 70%, optionally at least about 80%, preferably at least about 85%, more preferably at least about 90% and most preferably at least about 95% homologous to the sequence TVEGPLEDPSELEDHTSTPNP in HUMANK_P23 (SEQ ID NO:570).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMANK_P23 (SEQ ID NO:570) also has the following non-silent SNPs (Single Nucleotide Polymorphisms) as listed in Table 14, (given according to their position(s) on the amino acid sequence, with the alternative amino acid(s) listed; the last column indicates whether the SNP is known or not; the presence of known SNPs in variant protein HUMANK_P23 (SEQ ID NO:570) sequence provides support for the deduced sequence of this variant protein according to the present invention).

TABLE 14
Amino acid mutations

| SNP position(s) on | | |
|---|---|---|
| amino acid sequence | Alternative amino acid(s) | Previously known SNP? |
| 12 | L -> P | No |
| 63 | T -> P | No |
| 82 | G -> | No |
| 82 | G -> V | No |
| 89 | Q -> P | No |

Variant protein HUMANK_P23 (SEQ ID NO:570) is encoded by the following transcript(s): HUMANK_T28 (SEQ ID NO:42), for which the sequence(s) is/are given at the end of the application. The coding portion of transcript HUMANK_T28 (SEQ ID NO:42) is shown in bold; this coding portion starts at position 2053 and ends at position 2487. The transcript also has the following SNPs as listed in Table 15 (given according to their position on the nucleotide sequence, with the alternative nucleic acid listed; the last column indicates whether the SNP is known or not; the presence of known SNPs in variant protein HUMANK_P23 (SEQ ID NO:570) sequence provides support for the deduced sequence of this variant protein according to the present invention).

TABLE 15
Nucleic acid SNPs

| SNP position on | | |
|---|---|---|
| nucleotide sequence | Alternative nucleic acid | Previously known SNP? |
| 1603 | C -> T | No |
| 1969 | A -> G | No |
| 2005 | -> G | No |
| 2014 | C -> A | No |
| 2087 | T -> C | No |
| 2238 | C -> A | No |
| 2239 | A -> C | No |
| 2297 | G -> | No |
| 2297 | G -> T | No |
| 2318 | A -> C | No |
| 2500 | -> C | No |
| 2500 | -> T | No |
| 2700 | A -> | No |
| 2700 | A -> C | No |
| 2709 | A -> C | No |
| 2739 | A -> C | No |
| 2763 | A -> | No |
| 2763 | A -> C | No |
| 2857 | C -> T | No |
| 3272 | -> G | No |
| 3273 | -> C | No |
| 4085 | T -> A | Yes |
| 4142 | -> T | No |
| 4195 | G -> | No |
| 4570 | G -> T | Yes |
| 4979 | T -> | No |

Variant protein HUMANK_P27 (SEQ ID NO:571) according to the present invention has an amino acid sequence as given at the end of the application; it is encoded by transcript(s) HUMANK_T35 (SEQ ID NO:43). An alignment is given to the known protein (Ankyrin 1 (SEQ ID NO:628)) at the end of the application. One or more alignments to one or more previously published protein sequences are given at the end of the application. A brief description of the relationship of the variant protein according to the present invention to each such aligned protein is as follows:

Comparison Report Between HUMANK_P27 (SEQ ID NO:571) and AAH07930 (SEQ ID NO:631):

1. An isolated chimeric polypeptide encoding for HUMANK_P27 (SEQ ID NO:571), comprising a first amino acid sequence being at least 90% homologous to MWTFVTQLLVTLVLLSFFLVSCQNVMHIVRGSLCFVLKHIHQELDKELGESEGLSDDEETISTRVVRRRVF LKGNEFQNIPGEQVTEEQFTDEQGNIVTKK corresponding to amino acids 1-101 of AAH07930 (SEQ ID NO:631), which also corresponds to amino acids 1-101 of HUMANK_P27 (SEQ ID NO:571), and a second amino acid sequence being at least 70%, optionally at least 80%, preferably at least 85%, more preferably at least 90% and most preferably at least 95% homologous to a polypeptide having the sequence VGAECSPLCWGEAGGLEAKRW (SEQ ID NO:1516) corresponding to amino acids 102-122 of HUMANK_P27 (SEQ ID NO:571), wherein said first amino acid sequence and second amino acid sequence are contiguous and in a sequential order.

2. An isolated polypeptide encoding for a tail of HUMANK_P27 (SEQ ID NO:571), comprising a polypeptide being at least 70%, optionally at least about 80%, preferably at least about 85%, more preferably at least about 90% and most preferably at least about 95% homologous to the sequence VGAECSPLCWGEAGGLEAKRW (SEQ ID NO:1516) in HUMANK_P27 (SEQ ID NO:571).

Comparison Report Between HUMANK_P27 (SEQ ID NO:571) and Q8N604 (SEQ ID NO:630):

1. An isolated chimeric polypeptide encoding for HUMANK_P27 (SEQ ID NO:571), comprising a first amino acid sequence being at least 90% homologous to MWTFVTQLLVTLVLLSFFLVSCQNVMHIVRGSLCFVLKHIHQELDKELGESE corresponding to amino acids 1-52 of Q8N604 (SEQ ID NO:630), which also corresponds to amino acids 1-52 of HUMANK_P27 (SEQ ID NO:571), a bridging amino acid G corresponding to amino acid 53 of HUMANK_P27 (SEQ ID NO:571), a second amino acid sequence being at least 90% homologous to LSDDEETISTRVVRRRVFLKGNEFQNIPGEQVTEEQFTDEQGNIVTKK corresponding to amino acids 54-101 of Q8N604 (SEQ ID NO:630), which also corresponds to amino acids 54-101 of HUMANK_P27 (SEQ ID NO:571), and a third amino acid sequence being at least 70%, optionally at least 80%, preferably at least 85%, more preferably at least 90% and most preferably at least 95% homologous to a polypeptide having the sequence VGAECSPLCWGEAGGLEAKRW (SEQ ID NO:1516) corresponding to amino acids 102-122 of HUMANK_P27 (SEQ ID NO:571), wherein said first amino acid sequence, bridging amino acid, second amino acid sequence and third amino acid sequence are contiguous and in a sequential order.

2. An isolated polypeptide encoding for a tail of HUMANK_P27 (SEQ ID NO:571), comprising a polypeptide being at least 70%, optionally at least about 80%, preferably at least about 85%, more preferably at least about 90% and most preferably at least about 95% homologous to the sequence VGAECSPLCWGEAGGLEAKRW (SEQ ID NO:1516) in HUMANK_P27 (SEQ ID NO:571).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMANK_P27 (SEQ ID NO:571) also has the following non-silent SNPs (Single Nucleotide Polymorphisms) as listed in Table 16, (given according to their position(s) on the amino acid sequence, with the alternative amino acid(s) listed; the last column indicates whether the SNP is known or not; the presence of known SNPs in variant protein HUMANK_P27 (SEQ ID NO:571) sequence provides support for the deduced sequence of this variant protein according to the present invention).

TABLE 16
Amino acid mutations

| SNP position(s) on | | |
|---|---|---|
| amino acid sequence | Alternative amino acid(s) | Previously known SNP? |
| 12 | L -> P | No |
| 63 | T -> P | No |
| 82 | G -> | No |
| 82 | G -> V | No |
| 89 | Q -> P | No |

Variant protein HUMANK_P27 (SEQ ID NO:571) is encoded by the following transcript(s): HUMANK_T35 (SEQ ID NO:43), for which the sequence(s) is/are given at the end of the application. The coding portion of transcript HUMANK_T35 (SEQ ID NO:43) is shown in bold; this coding portion starts at position 2053 and ends at position 2418. The transcript also has the following SNPs as listed in Table 17 (given according to their position on the nucleotide sequence, with the alternative nucleic acid listed; the last column indicates whether the SNP is known or not; the presence of known SNPs in variant protein HUMANK_P27 (SEQ ID NO:571) sequence provides support for the deduced sequence of this variant protein according to the present invention).

TABLE 17
Nucleic acid SNPs

| SNP position on | | |
|---|---|---|
| nucleotide sequence | Alternative nucleic acid | Previously known SNP? |
| 1603 | C -> T | No |
| 1969 | A -> G | No |
| 2005 | -> G | No |
| 2014 | C -> A | No |
| 2087 | T -> C | No |
| 2238 | C -> A | No |
| 2239 | A -> C | No |
| 2297 | G -> | No |
| 2297 | G -> T | No |
| 2318 | A -> C | No |

Variant protein HUMANK_P29 (SEQ ID NO:572) according to the present invention has an amino acid sequence as given at the end of the application; it is encoded by transcript(s) HUMANK_T3 (SEQ ID NO:36). An alignment is given to the known protein (Ankyrin 1 (SEQ ID NO:628)) at the end of the application. One or more alignments to one or more previously published protein sequences are given at the end of the application. A brief description of the relationship of the variant protein according to the present invention to each such aligned protein is as follows:

Comparison Report Between HUMANK_P29 (SEQ ID NO:572) and AAH07930 (SEQ ID NO:631):

1. An isolated chimeric polypeptide encoding for HUMANK_P29 (SEQ ID NO:572), comprising a first amino acid sequence being at least 90% homologous to MWTFVTQLLVTLVLLSFFLVSCQNVMHIVRGSLCFVLKHIHQELDKELGESEGLSDDEETIS corresponding to amino acids 1-62 of AAH07930 (SEQ ID NO:631), which also corresponds to amino acids 1-62 of HUMANK_P29 (SEQ ID NO:572), a bridging amino acid P corresponding to amino acid 63 of HUMANK_P29 (SEQ ID NO:572), a second amino acid sequence being at least 90% homologous to RVVRRRVFLKGNEFQNIPGEQVTEEQFTDEQGNIVTKKIIRKVVRQIDLSSADAAQEHEE corresponding to amino acids 64-123 of AAH07930 (SEQ ID NO:631), which also corresponds to amino acids 64-123 of HUMANK_P29 (SEQ ID NO:572), and a third amino acid sequence being at least 70%, optionally at least 80%, preferably at least 85%, more preferably at least 90% and most preferably at least 95% homologous to a polypeptide having the sequence VELRGSGLQPDLIEGRKGAQIVKRASLKRGKQ (SEQ ID NO:1517) corresponding to amino acids 124-155 of HUMANK_P29 (SEQ ID NO:572), wherein said first amino acid sequence, bridging amino acid, second amino acid sequence and third amino acid sequence are contiguous and in a sequential order.

2. An isolated polypeptide encoding for a tail of HUMANK_P29 (SEQ ID NO:572), comprising a polypeptide being at least 70%, optionally at least about 80%, preferably at least about 85%, more preferably at least about 90% and most preferably at least about 95% homologous to the sequence VELRGSGLQPDLIEGRKGAQIVKRASLKRGKQ (SEQ ID NO:1517) in HUMANK_P29 (SEQ ID NO:572).

Comparison Report Between HUMANK_P29 (SEQ ID NO:572) and Q8N604 (SEQ ID NO:630):

1. An isolated chimeric polypeptide encoding for HUMANK_P29 (SEQ ID NO:572), comprising a first amino acid sequence being at least 90% homologous to MWTFVTQLLVTLVLLSFFLVSCQNVMHIVRGSLCFVLKHIHQELDKELGESE corresponding to amino acids 1-52 of Q8N604 (SEQ ID NO:630), which also corresponds to amino acids 1-52 of HUMANK_P29 (SEQ ID NO:572), a bridging amino acid G corresponding to amino acid 53 of HUMANK_P29 (SEQ ID NO:572), a second amino acid sequence being at least 90% homologous to LSDDEETIS corresponding to amino acids 54-62 of Q8N604 (SEQ ID NO:630), which also corresponds to amino acids 54-62 of HUMANK_P29 (SEQ ID NO:572), a bridging amino acid P corresponding to amino acid 63 of HUMANK_P29 (SEQ ID NO:572), and a third amino acid sequence being at least 90% homologous to RVVRRRVFLKGNEFQNIPGEQVTEEQFTDEQGNIVTKKIIRKVVRQIDLSSADAAQEHEEVELRGSGLQPDL IEGRKGAQIVKRASLKRGKQ corresponding to amino acids 64-155 of Q8N604 (SEQ ID NO:630), which also corresponds to amino acids 64-155 of HUMANK_P29 (SEQ ID NO:572), wherein said first amino acid sequence, bridging amino acid, second amino acid sequence, bridging amino acid and third amino acid sequence are contiguous and in a sequential order.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMANK_P29 (SEQ ID NO:572) also has the following non-silent SNPs (Single Nucleotide Polymorphisms) as listed in Table 18, (given according to their position(s) on the amino acid sequence, with the alternative amino acid(s) listed; the last column indicates whether the SNP is known or not; the presence of known SNPs in variant protein HUMANK_P29 (SEQ ID NO:572) sequence provides support for the deduced sequence of this variant protein according to the present invention).

TABLE 18
Amino acid mutations

| SNP position(s) on | | |
|---|---|---|
| amino acid sequence | Alternative amino acid(s) | Previously known SNP? |
| 12 | L -> P | No |
| 63 | P -> T | No |
| 82 | G -> | No |
| 82 | G -> V | No |
| 89 | Q -> P | No |

Variant protein HUMANK_P29 (SEQ ID NO:572) is encoded by the following transcript(s): HUMANK_T3 (SEQ ID NO:36), for which the sequence(s) is/are given at the end of the application. The coding portion of transcript HUMANK_T3 (SEQ ID NO:36) is shown in bold; this coding portion starts at position 2053 and ends at position 2517. The transcript also has the following SNPs as listed in Table 19 (given according to their position on the nucleotide sequence, with the alternative nucleic acid listed; the last column indicates whether the SNP is known or not; the presence of known SNPs in variant protein HUMANK_P29 (SEQ ID NO:572) sequence provides support for the deduced sequence of this variant protein according to the present invention).

TABLE 19
Nucleic acid SNPs

| SNP position on | | |
|---|---|---|
| nucleotide sequence | Alternative nucleic acid | Previously known SNP? |
| 1603 | C -> T | No |
| 1969 | A -> G | No |
| 2005 | -> G | No |
| 2014 | C -> A | No |
| 2087 | T -> C | No |
| 2238 | C -> A | No |
| 2239 | A -> C | No |
| 2297 | G -> | No |
| 2297 | G -> T | No |
| 2318 | A -> C | No |
| 2593 | -> C | No |
| 2593 | -> T | No |
| 2793 | A -> | No |
| 2793 | A -> C | No |
| 2802 | A -> C | No |
| 2832 | A -> C | No |
| 2856 | A -> | No |
| 2856 | A -> C | No |
| 2950 | C -> T | No |
| 3242 | G -> T | Yes |
| 3244 | C -> A | Yes |
| 3245 | T -> A | Yes |
| 3246 | C -> T | Yes |
| 3379 | -> G | No |
| 3380 | -> C | No |
| 4192 | T -> A | Yes |
| 4249 | -> T | No |
| 4302 | G -> | No |
| 4677 | G -> T | Yes |
| 5086 | T -> | No |

Variant protein HUMANK_P33 (SEQ ID NO:573) according to the present invention has an amino acid sequence as given at the end of the application; it is encoded by transcript(s) HUMANK_T23 (SEQ ID NO:38). An alignment is given to the known protein (Ankyrin 1 (SEQ ID NO:628)) at the end of the application. One or more alignments to one or more previously published protein sequences are given at the end of the application. A brief description of the relationship of the variant protein according to the present invention to each such aligned protein is as follows:

Comparison Report Between HUMANK_P33 (SEQ ID NO:573) and AAH07930 (SEQ ID NO:631):

1. An isolated chimeric polypeptide encoding for HUMANK_P33 (SEQ ID NO:573), comprising a first amino acid sequence being at least 90% homologous to MWTFVTQLLVTLVLLSFFLVSCQNVMHIVRGSLCFVLKHIHQELDKELGESEGLSDDEETIS corresponding to amino acids 1-62 of AAH07930 (SEQ ID NO:631), which also corresponds to amino acids 1-62 of HUMANK_P33 (SEQ ID NO:573), a bridging amino acid P corresponding to amino acid 63 of HUMANK_P33 (SEQ ID NO:573), a second amino acid sequence being at least 90% homologous to RVVRRRVFLKGNEFQNIPGEQVTEEQFTDEQGNIVTKK corresponding to amino acids 64-101 of AAH07930 (SEQ ID NO:631), which also corresponds to amino acids 64-101 of HUMANK_P33 (SEQ ID NO:573), and a third amino acid sequence being at least 70%, optionally at least 80%, preferably at least 85%, more preferably at least 90% and most preferably at least 95% homologous to a polypeptide having the sequence DHTSTPNP (SEQ ID NO:1518) corresponding to amino acids 102-109 of HUMANK_P33 (SEQ ID NO:573), wherein said first amino acid sequence, bridging amino acid, second amino acid sequence and third amino acid sequence are contiguous and in a sequential order.

2. An isolated polypeptide encoding for a tail of HUMANK_P33 (SEQ ID NO:573), comprising a polypeptide being at least 70%, optionally at least about 80%, preferably at least about 85%, more preferably at least about 90% and most preferably at least about 95% homologous to the sequence DHTSTPNP (SEQ ID NO:1518) in HUMANK_P33 (SEQ ID NO:573).

Comparison Report Between HUMANK_P33 (SEQ ID NO:573) and Q8N604 (SEQ ID NO:630):

1. An isolated chimeric polypeptide encoding for HUMANK_P33 (SEQ ID NO:573), comprising a first amino acid sequence being at least 90% homologous to MWTFVTQLLVTLVLLSFFLVSCQNVMHIVRGSLCFVLKHIHQELDKELGESE corresponding to amino acids 1-52 of Q8N604 (SEQ ID NO:630), which also corresponds to amino acids 1-52 of HUMANK_P33 (SEQ ID NO:573), a bridging amino acid G corresponding to amino acid 53 of HUMANK_P33 (SEQ ID NO:573), a second amino acid sequence being at least 90% homologous to LSDDEETIS corresponding to amino acids 54-62 of Q8N604 (SEQ ID NO:630), which also corresponds to amino acids 54-62 of HUMANK_P33 (SEQ ID NO:573), a bridging amino acid P corresponding to amino acid 63 of HUMANK_P33 (SEQ ID NO:573), a third amino acid sequence being at least 90% homologous to RVVRRRVFLKGNEFQNIPGEQVTEEQFTDEQGNIVTKK corresponding to amino acids 64-101 of Q8N604 (SEQ ID NO:630), which also corresponds to amino acids 64-101 of HUMANK_P33 (SEQ ID NO:573), and a fourth amino acid sequence being at least 70%, optionally at least 80%, preferably at least 85%, more preferably at least 90% and most preferably at least 95% homologous to a polypeptide having the sequence DHTSTPNP (SEQ ID NO:1518) corresponding to amino acids 102-109 of HUMANK_P33 (SEQ ID NO:573), wherein said first amino acid sequence, bridging amino acid, second amino acid sequence, bridging amino acid, third amino acid sequence and fourth amino acid sequence are contiguous and in a sequential order.

2. An isolated polypeptide encoding for a tail of HUMANK_P33 (SEQ ID NO:573), comprising a polypeptide being at least 70%, optionally at least about 80%, preferably at least about 85%, more preferably at least about 90% and most preferably at least about 95% homologous to the sequence DHTSTPNP (SEQ ID NO:1518) in HUMANK_P33 (SEQ ID NO:573).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMANK_P33 (SEQ ID NO:573) also has the following non-silent SNPs (Single Nucleotide Polymorphisms) as listed in Table 20, (given according to their position(s) on the amino acid sequence, with the alternative amino acid(s) listed; the last column indicates whether the SNP is known or not; the presence of known SNPs in variant protein HUMANK_P33 (SEQ ID NO:573) sequence provides support for the deduced sequence of this variant protein according to the present invention).

TABLE 20
Amino acid mutations

| SNP position(s) on | | |
|---|---|---|
| amino acid sequence | Alternative amino acid(s) | Previously known SNP? |
| 12 | L -> P | No |
| 63 | P -> T | No |
| 82 | G -> | No |
| 82 | G -> V | No |
| 89 | Q -> P | No |

Variant protein HUMANK_P33 (SEQ ID NO:573) is encoded by the following transcript(s): HUMANK_T23 (SEQ ID NO:38), for which the sequence(s) is/are given at the end of the application. The coding portion of transcript HUMANK_T23 (SEQ ID NO:38) is shown in bold; this coding portion starts at position 2053 and ends at position 2379. The transcript also has the following SNPs as listed in Table 21 (given according to their position on the nucleotide sequence, with the alternative nucleic acid listed; the last column indicates whether the SNP is known or not; the presence of known SNPs in variant protein HUMANK_P33 (SEQ ID NO:573) sequence provides support for the deduced sequence of this variant protein according to the present invention).

TABLE 21
Nucleic acid SNPs

| SNP position on | | |
|---|---|---|
| nucleotide sequence | Alternative nucleic acid | Previously known SNP? |
| 1603 | C -> T | No |
| 1969 | A -> G | No |
| 2005 | -> G | No |
| 2014 | C -> A | No |
| 2087 | T -> C | No |
| 2238 | C -> A | No |
| 2239 | A -> C | No |
| 2297 | G -> | No |
| 2297 | G -> T | No |
| 2318 | A -> C | No |
| 2392 | -> C | No |
| 2392 | -> T | No |
| 2592 | A -> | No |
| 2592 | A -> C | No |
| 2601 | A -> C | No |
| 2631 | A -> C | No |
| 2655 | A -> | No |
| 2655 | A -> C | No |
| 2749 | C -> T | No |
| 3164 | -> G | No |
| 3165 | -> C | No |
| 3977 | T -> A | Yes |
| 4034 | -> T | No |
| 4087 | G -> | No |
| 4462 | G -> T | Yes |
| 4871 | T -> | No |

Variant protein HUMANK_P34 (SEQ ID NO:574) according to the present invention has an amino acid sequence as given at the end of the application; it is encoded by transcript(s) HUMANK_T24 (SEQ ID NO:39). An alignment is given to the known protein (Ankyrin 1 (SEQ ID NO:628)) at the end of the application. One or more alignments to one or more previously published protein sequences are given at the end of the application. A brief description of the relationship of the variant protein according to the present invention to each such aligned protein is as follows:

Comparison Report Between HUMANK_P34 (SEQ ID NO:574) and AAH07930 (SEQ ID NO:631):

1. An isolated chimeric polypeptide encoding for HUMANK_P34 (SEQ ID NO:574), comprising a first amino acid sequence being at least 90% homologous to MWTFVTQLLVTLVLLSFFLVSCQNVMHIVRGSLCFVLKHIHQELDKELGESEGLSDDEETIS corresponding to amino acids 1-62 of AAH07930 (SEQ ID NO:631), which also corresponds to amino acids 1-62 of HUMANK_P34 (SEQ ID NO:574), a bridging amino acid P corresponding to amino acid 63 of HUMANK_P34 (SEQ ID NO:574), a second amino acid sequence being at least 90% homologous to RVVRRRVFLKGNEFQNIPGEQVTEEQFTDEQGNIVTKK corresponding to amino acids 64-101 of AAH07930 (SEQ ID NO:631), which also corresponds to amino acids 64-101 of HUMANK_P34 (SEQ ID NO:574), and a third amino acid sequence being at least 70%, optionally at least 80%, preferably at least 85%, more preferably at least 90% and most preferably at least 95% homologous to a polypeptide having the sequence VELRGSGLQPDLIEGRKGAQIVKRASLKRGKQ (SEQ ID NO:1517) corresponding to amino acids 102-133 of HUMANK_P34 (SEQ ID NO:574), wherein said first amino acid sequence, bridging amino acid, second amino acid sequence and third amino acid sequence are contiguous and in a sequential order.

2. An isolated polypeptide encoding for a tail of HUMANK_P34 (SEQ ID NO:574), comprising a polypeptide being at least 70%, optionally at least about 80%, preferably at least about 85%, more preferably at least about 90% and most preferably at least about 95% homologous to the sequence VELRGSGLQPDLIEGRKGAQIVKRASLKRGKQ (SEQ ID NO:1517) in HUMANK_P34 (SEQ ID NO:574).

Comparison Report Between HUMANK_P34 (SEQ ID NO:574) and Q8N604 (SEQ ID NO:630):

1. An isolated chimeric polypeptide encoding for HUMANK_P34 (SEQ ID NO:574), comprising a first amino acid sequence being at least 90% homologous to MWTFVTQLLVTLVLLSFFLVSCQNVMHIVRGSLCFVLKHIHQELDKELGESE corresponding to amino acids 1-52 of Q8N604 (SEQ ID NO:630), which also corresponds to amino acids 1-52 of HUMANK_P34 (SEQ ID NO:574), a bridging amino acid G corresponding to amino acid 53 of HUMANK_P34 (SEQ ID NO:574), a second amino acid sequence being at least 90% homologous to LSDDEETIS corresponding to amino acids 54-62 of Q8N604 (SEQ ID NO:630), which also corresponds to amino acids 54-62 of HUMANK_P34 (SEQ ID NO:574), a bridging amino acid P corresponding to amino acid 63 of HUMANK_P34 (SEQ ID NO:574), a third amino acid sequence being at least 90% homologous to RVVRRRVFLKGNEFQNIPGEQVTEEQFTDEQGNIVTKK corresponding to amino acids 64-101 of Q8N604 (SEQ ID NO:630), which also corresponds to amino acids 64-101 of HUMANK_P34 (SEQ ID NO:574), and a fourth amino acid sequence being at least 90% homologous to VELRGSGLQPDLIEGRKGAQIVKRASLKRGKQ (SEQ ID NO:1517) corresponding to amino acids 124-155 of Q8N604 (SEQ ID NO:630), which also corresponds to amino acids 102-133 of HUMANK_P34 (SEQ ID NO:574), wherein said first amino acid sequence, bridging amino acid, second amino acid sequence, bridging amino acid, third amino acid sequence and fourth amino acid sequence are contiguous and in a sequential order.

2. An isolated chimeric polypeptide encoding for an edge portion of HUMANK_P34 (SEQ ID NO:574), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KV, having a structure as follows: a sequence starting from any of amino acid numbers 101−x to 101; and ending at any of amino acid numbers 102+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMANK_P34 (SEQ ID NO:574) also has the following non-silent SNPs (Single Nucleotide Polymorphisms) as listed in Table 22, (given according to their position(s) on the amino acid sequence, with the alternative amino acid(s) listed; the last column indicates whether the SNP is known or not; the presence of known SNPs in variant protein HUMANK_P34 (SEQ ID NO:574) sequence provides support for the deduced sequence of this variant protein according to the present invention).

TABLE 22
Amino acid mutations

| SNP position(s) on | | |
|---|---|---|
| amino acid sequence | Alternative amino acid(s) | Previously known SNP? |
| 12 | L -> P | No |
| 63 | P -> T | No |
| 82 | G -> | No |
| 82 | G -> V | No |
| 89 | Q -> P | No |

Variant protein HUMANK_P34 (SEQ ID NO:574) is encoded by the following transcript(s): HUMANK_T24 (SEQ ID NO:39), for which the sequence(s) is/are given at the end of the application. The coding portion of transcript HUMANK_T24 (SEQ ID NO:39) is shown in bold; this coding portion starts at position 2053 and ends at position 2451. The transcript also has the following SNPs as listed in Table 23 (given according to their position on the nucleotide sequence, with the alternative nucleic acid listed; the last column indicates whether the SNP is known or not; the presence of known SNPs in variant protein HUMANK_P34 (SEQ ID NO:574) sequence provides support for the deduced sequence of this variant protein according to the present invention).

TABLE 23
Nucleic acid SNPs

| SNP position on | | |
|---|---|---|
| nucleotide sequence | Alternative nucleic acid | Previously known SNP? |
| 1603 | C -> T | No |
| 1969 | A -> G | No |
| 2005 | -> G | No |
| 2014 | C -> A | No |
| 2087 | T -> C | No |
| 2238 | C -> A | No |
| 2239 | A -> C | No |
| 2297 | G -> | No |
| 2297 | G -> T | No |
| 2318 | A -> C | No |
| 2527 | -> C | No |
| 2527 | -> T | No |
| 2727 | A -> | No |
| 2727 | A -> C | No |
| 2736 | A -> C | No |
| 2766 | A -> C | No |
| 2790 | A -> | No |
| 2790 | A -> C | No |
| 2884 | C -> T | No |
| 3299 | -> G | No |
| 3300 | -> C | No |
| 4112 | T -> A | Yes |
| 4169 | -> T | No |
| 4222 | G -> | No |
| 4597 | G -> T | Yes |
| 5006 | T -> | No |

As noted above, cluster HUMANK features 22 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HUMANK_node_{—}91 (SEQ ID NO:285) according to the present invention is supported by 8 libraries. The number of libraries was determined as previously described. This segment can be found in the following transcript(s): HUMANK_T3 (SEQ ID NO:36), HUMANK_T13 (SEQ ID NO:37), HUMANK_T23 (SEQ ID NO:38), HUMANK_T24 (SEQ ID NO:39), HUMANK_T26 (SEQ ID NO:40), HUMANK_T27 (SEQ ID NO:41), HUMANK_T28 (SEQ ID NO:42) and HUMANK_T35 (SEQ ID NO:43). Table 24 below describes the starting and ending position of this segment on each transcript.

TABLE 24
Segment location on transcripts

| | Segment | Segment |
|---|---|---|
| Transcript name | starting position | ending position |
| HUMANK_T3 (SEQ ID NO: 36) | 1 | 1543 |
| HUMANK_T13 (SEQ ID NO: 37) | 1 | 1543 |
| HUMANK_T23 (SEQ ID NO: 38) | 1 | 1543 |
| HUMANK_T24 (SEQ ID NO: 39) | 1 | 1543 |
| HUMANK_T26 (SEQ ID NO: 40) | 1 | 1543 |
| HUMANK_T27 (SEQ ID NO: 41) | 1 | 1543 |
| HUMANK_T28 (SEQ ID NO: 42) | 1 | 1543 |
| HUMANK_T35 (SEQ ID NO: 43) | 1 | 1543 |

Segment cluster HUMANK_node_{—}92 (SEQ ID NO:286) according to the present invention is supported by 19 libraries. The number of libraries was determined as previously described. This segment can be found in the following transcript(s): HUMANK_T3 (SEQ ID NO:36), HUMANK_T13 (SEQ ID NO:37), HUMANK_T23 (SEQ ID NO:38), HUMANK_T24 (SEQ ID NO:39), HUMANK_T26 (SEQ ID NO:40), HUMANK_T27 (SEQ ID NO:41), HUMANK_T28 (SEQ ID NO:42) and HUMANK_T35 (SEQ ID NO:43). Table 25 below describes the starting and ending position of this segment on each transcript.

TABLE 25
Segment location on transcripts

| | Segment | Segment |
|---|---|---|
| Transcript name | starting position | ending position |
| HUMANK_T3 (SEQ ID NO: 36) | 1544 | 1928 |
| HUMANK_T13 (SEQ ID NO: 37) | 1544 | 1928 |
| HUMANK_T23 (SEQ ID NO: 38) | 1544 | 1928 |
| HUMANK_T24 (SEQ ID NO: 39) | 1544 | 1928 |
| HUMANK_T26 (SEQ ID NO: 40) | 1544 | 1928 |
| HUMANK_T27 (SEQ ID NO: 41) | 1544 | 1928 |
| HUMANK_T28 (SEQ ID NO: 42) | 1544 | 1928 |
| HUMANK_T35 (SEQ ID NO: 43) | 1544 | 1928 |

Segment cluster HUMANK_node_{—}93 (SEQ ID NO:287) according to the present invention is supported by 31 libraries. The number of libraries was determined as previously described. This segment can be found in the following transcript(s): HUMANK_T3 (SEQ ID NO:36), HUMANK_T13 (SEQ ID NO:37), HUMANK_T23 (SEQ ID NO:38), HUMANK_T24 (SEQ ID NO:39), HUMANK_T26 (SEQ ID NO:40), HUMANK_T27 (SEQ ID NO:41), HUMANK_T28 (SEQ ID NO:42) and HUMANK_T35 (SEQ ID NO:43). Table 26 below describes the starting and ending position of this segment on each transcript.

TABLE 26
Segment location on transcripts

| | Segment | Segment |
|---|---|---|
| Transcript name | starting position | ending position |
| HUMANK_T3 (SEQ ID NO: 36) | 1929 | 2178 |
| HUMANK_T13 (SEQ ID NO: 37) | 1929 | 2178 |
| HUMANK_T23 (SEQ ID NO: 38) | 1929 | 2178 |
| HUMANK_T24 (SEQ ID NO: 39) | 1929 | 2178 |
| HUMANK_T26 (SEQ ID NO: 40) | 1929 | 2178 |
| HUMANK_T27 (SEQ ID NO: 41) | 1929 | 2178 |
| HUMANK_T28 (SEQ ID NO: 42) | 1929 | 2178 |
| HUMANK_T35 (SEQ ID NO: 43) | 1929 | 2178 |

Segment cluster HUMANK_node_{—}100 (SEQ ID NO:288) according to the present invention is supported by 1 libraries. The number of libraries was determined as previously described. This segment can be found in the following transcript(s): HUMANK_T35 (SEQ ID NO:43). Table 27 below describes the starting and ending position of this segment on each transcript.

TABLE 27
Segment location on transcripts

| | Segment | Segment |
|---|---|---|
| Transcript name | starting position | ending position |
| HUMANK_T35 (SEQ ID NO: 43) | 2356 | 2490 |

Segment cluster HUMANK_node_{—}108 (SEQ ID NO:289) according to the present invention is supported by 33 libraries. The number of libraries was determined as previously described. This segment can be found in the following transcript(s): HUMANK_T3 (SEQ ID NO:36), HUMANK_T24 (SEQ ID NO:39), HUMANK_T26 (SEQ ID NO:40) and HUMANK_T27 (SEQ ID NO:41). Table 28 below describes the starting and ending position of this segment on each transcript.

TABLE 28
Segment location on transcripts

| | Segment | Segment |
|---|---|---|
| Transcript name | starting position | ending position |
| HUMANK_T3 (SEQ ID NO: 36) | 2422 | 2556 |
| HUMANK_T24 (SEQ ID NO: 39) | 2356 | 2490 |
| HUMANK_T26 (SEQ ID NO: 40) | 2464 | 2598 |
| HUMANK_T27 (SEQ ID NO: 41) | 2497 | 2631 |

Segment cluster HUMANK_node_{—}113 (SEQ ID NO:290) according to the present invention is supported by 56 libraries. The number of libraries was determined as previously described. This segment can be found in the following transcript(s): HUMANK_T3 (SEQ ID NO:36), HUMANK_T13 (SEQ ID NO:37), HUMANK_T23 (SEQ ID NO:38), HUMANK_T24 (SEQ ID NO:39), HUMANK_T26 (SEQ ID NO:40), HUMANK_T27 (SEQ ID NO:41) and HUMANK_T28 (SEQ ID NO:42). Table 29 below describes the starting and ending position of this segment on each transcript.

TABLE 29
Segment location on transcripts

| | Segment | Segment |
|---|---|---|
| Transcript name | starting position | ending position |
| HUMANK_T3 (SEQ ID NO: 36) | 2596 | 2735 |
| HUMANK_T13 (SEQ ID NO: 37) | 2536 | 2675 |
| HUMANK_T23 (SEQ ID NO: 38) | 2395 | 2534 |
| HUMANK_T24 (SEQ ID NO: 39) | 2530 | 2669 |
| HUMANK_T26 (SEQ ID NO: 40) | 2638 | 2777 |
| HUMANK_T27 (SEQ ID NO: 41) | 2671 | 2810 |
| HUMANK_T28 (SEQ ID NO: 42) | 2503 | 2642 |

Segment cluster HUMANK_node_{—}115 (SEQ ID NO:291) according to the present invention is supported by 63 libraries. The number of libraries was determined as previously described. This segment can be found in the following transcript(s): HUMANK_T3 (SEQ ID NO:36), HUMANK_T13 (SEQ ID NO:37), HUMANK_T23 (SEQ ID NO:38), HUMANK_T24 (SEQ ID NO:39), HUMANK_T26 (SEQ ID NO:40), HUMANK_T27 (SEQ ID NO:41) and HUMANK T28 (SEQ ID NO:42). Table 30 below describes the starting and ending position of this segment on each transcript.

TABLE 30
Segment location on transcripts

| | Segment | Segment |
|---|---|---|
| Transcript name | starting position | ending position |
| HUMANK_T3 (SEQ ID NO: 36) | 2821 | 3234 |
| HUMANK_T13 (SEQ ID NO: 37) | 2761 | 3174 |
| HUMANK_T23 (SEQ ID NO: 38) | 2620 | 3033 |
| HUMANK_T24 (SEQ ID NO: 39) | 2755 | 3168 |
| HUMANK_T26 (SEQ ID NO: 40) | 2863 | 3276 |
| HUMANK_T27 (SEQ ID NO: 41) | 2896 | 3309 |
| HUMANK_T28 (SEQ ID NO: 42) | 2728 | 3141 |

Segment cluster HUMANK_node_{—}117 (SEQ ID NO:292) according to the present invention is supported by 28 libraries. The number of libraries was determined as previously described. This segment can be found in the following transcript(s): HUMANK_T3 (SEQ ID NO:36), HUMANK_T13 (SEQ ID NO:37), HUMANK_T23 (SEQ ID NO:38), HUMANK_T24 (SEQ ID NO:39), HUMANK_T26 (SEQ ID NO:40), HUMANK_T27 (SEQ ID NO:41) and HUMANK_T28 (SEQ ID NO:42). Table 31 below describes the starting and ending position of this segment on each transcript.

TABLE 31
Segment location on transcripts

| | Segment | Segment |
|---|---|---|
| Transcript name | starting position | ending position |
| HUMANK_T3 (SEQ ID NO: 36) | 3249 | 4438 |
| HUMANK_T13 (SEQ ID NO: 37) | 3175 | 4364 |
| HUMANK_T23 (SEQ ID NO: 38) | 3034 | 4223 |
| HUMANK_T24 (SEQ ID NO: 39) | 3169 | 4358 |
| HUMANK_T26 (SEQ ID NO: 40) | 3277 | 4466 |
| HUMANK_T27 (SEQ ID NO: 41) | 3310 | 4499 |
| HUMANK_T28 (SEQ ID NO: 42) | 3142 | 4331 |

Segment cluster HUMANK_node_{—}119 (SEQ ID NO:293) according to the present invention is supported by 10 libraries. The number of libraries was determined as previously described. This segment can be found in the following transcript(s): HUMANK_T3 (SEQ ID NO:36), HUMANK_T13 (SEQ ID NO:37), HUMANK_T23 (SEQ ID NO:38), HUMANK_T24 (SEQ ID NO:39), HUMANK_T26 (SEQ ID NO:40), HUMANK_T27 (SEQ ID NO:41) and HUMANK_T28 (SEQ ID NO:42). Table 32 below describes the starting and ending position of this segment on each transcript.

TABLE 32
Segment location on transcripts

| | Segment | Segment |
|---|---|---|
| Transcript name | starting position | ending position |
| HUMANK_T3 (SEQ ID NO: 36) | 4439 | 4797 |
| HUMANK_T13 (SEQ ID NO: 37) | 4365 | 4723 |
| HUMANK_T23 (SEQ ID NO: 38) | 4224 | 4582 |
| HUMANK_T24 (SEQ ID NO: 39) | 4359 | 4717 |
| HUMANK_T26 (SEQ ID NO: 40) | 4467 | 4825 |
| HUMANK_T27 (SEQ ID NO: 41) | 4500 | 4858 |
| HUMANK_T28 (SEQ ID NO: 42) | 4332 | 4690 |

Segment cluster HUMANK_node_{—}120 (SEQ ID NO:294) according to the present invention is supported by 13 libraries. The number of libraries was determined as previously described. This segment can be found in the following transcript(s): HUMANK_T3 (SEQ ID NO:36), HUMANK_T13 (SEQ ID NO:37), HUMANK_T23 (SEQ ID NO:38), HUMANK_T24 (SEQ ID NO:39), HUMANK_T26 (SEQ ID NO:40), HUMANK_T27 (SEQ ID NO:41) and HUMANK_T28 (SEQ ID NO:42). Table 33 below describes the starting and ending position of this segment on each transcript.

TABLE 33
Segment location on transcripts

| | Segment | Segment |
|---|---|---|
| Transcript name | starting position | ending position |
| HUMANK_T3 (SEQ ID NO: 36) | 4798 | 5100 |
| HUMANK_T13 (SEQ ID NO: 37) | 4724 | 5026 |
| HUMANK_T23 (SEQ ID NO: 38) | 4583 | 4885 |
| HUMANK_T24 (SEQ ID NO: 39) | 4718 | 5020 |
| HUMANK_T26 (SEQ ID NO: 40) | 4826 | 5128 |
| HUMANK_T27 (SEQ ID NO: 41) | 4859 | 5161 |
| HUMANK_T28 (SEQ ID NO: 42) | 4691 | 4993 |

According to an optional embodiment of the present invention, short segments related to the above cluster are also provided. These segments are up to about 120 bp in length, and so are included in a separate description.

Segment cluster HUMANK_node_{—}94 (SEQ ID NO:295) according to the present invention is supported by 28 libraries. The number of libraries was determined as previously described. This segment can be found in the following transcript(s): HUMANK_T3 (SEQ ID NO:36), HUMANK_T13 (SEQ ID NO:37), HUMANK_T23 (SEQ ID NO:38), HUMANK_T24 (SEQ ID NO:39), HUMANK_T26 (SEQ ID NO:40), HUMANK_T27 (SEQ ID NO:41), HUMANK_T28 (SEQ ID NO:42) and HUMANK_T35 (SEQ ID NO:43). Table 34 below describes the starting and ending position of this segment on each transcript.

TABLE 34
Segment location on transcripts

| | Segment | Segment |
|---|---|---|
| Transcript name | starting position | ending position |
| HUMANK_T3 (SEQ ID NO: 36) | 2179 | 2240 |
| HUMANK_T13 (SEQ ID NO: 37) | 2179 | 2240 |
| HUMANK_T23 (SEQ ID NO: 38) | 2179 | 2240 |
| HUMANK_T24 (SEQ ID NO: 39) | 2179 | 2240 |
| HUMANK_T26 (SEQ ID NO: 40) | 2179 | 2240 |
| HUMANK_T27 (SEQ ID NO: 41) | 2179 | 2240 |
| HUMANK_T28 (SEQ ID NO: 42) | 2179 | 2240 |
| HUMANK_T35 (SEQ ID NO: 43) | 2179 | 2240 |

Segment cluster HUMANK_node_{—}95 (SEQ ID NO:296) according to the present invention is supported by 28 libraries. The number of libraries was determined as previously described. This segment can be found in the following transcript(s): HUMANK_T3 (SEQ ID NO:36), HUMANK_T13 (SEQ ID NO:37), HUMANK_T23 (SEQ ID NO:38), HUMANK_T24 (SEQ ID NO:39), HUMANK_T26 (SEQ ID NO:40), HUMANK_T27 (SEQ ID NO:41), HUMANK_T28 (SEQ ID NO:42) and HUMANK_T35 (SEQ ID NO:43). Table 35 below describes the starting and ending position of this segment on each transcript.

TABLE 35
Segment location on transcripts

| | Segment | Segment |
|---|---|---|
| Transcript name | starting position | ending position |
| HUMANK_T3 (SEQ ID NO: 36) | 2241 | 2271 |
| HUMANK_T13 (SEQ ID NO: 37) | 2241 | 2271 |
| HUMANK_T23 (SEQ ID NO: 38) | 2241 | 2271 |
| HUMANK_T24 (SEQ ID NO: 39) | 2241 | 2271 |
| HUMANK_T26 (SEQ ID NO: 40) | 2241 | 2271 |
| HUMANK_T27 (SEQ ID NO: 41) | 2241 | 2271 |
| HUMANK_T28 (SEQ ID NO: 42) | 2241 | 2271 |
| HUMANK_T35 (SEQ ID NO: 43) | 2241 | 2271 |

Segment cluster HUMANK_node_{—}98 (SEQ ID NO:297) according to the present invention is supported by 53 libraries. The number of libraries was determined as previously described. This segment can be found in the following transcript(s): HUMANK_T3 (SEQ ID NO:36), HUMANK_T13 (SEQ ID NO:37), HUMANK_T23 (SEQ ID NO:38), HUMANK_T24 (SEQ ID NO:39), HUMANK_T26 (SEQ ID NO:40), HUMANK_T27 (SEQ ID NO:41), HUMANK_T28 (SEQ ID NO:42) and HUMANK_T35 (SEQ ID NO:43). Table 36 below describes the starting and ending position of this segment on each transcript.

TABLE 36
Segment location on transcripts

| | Segment | Segment |
|---|---|---|
| Transcript name | starting position | ending position |
| HUMANK_T3 (SEQ ID NO: 36) | 2272 | 2348 |
| HUMANK_T13 (SEQ ID NO: 37) | 2272 | 2348 |
| HUMANK_T23 (SEQ ID NO: 38) | 2272 | 2348 |
| HUMANK_T24 (SEQ ID NO: 39) | 2272 | 2348 |
| HUMANK_T26 (SEQ ID NO: 40) | 2272 | 2348 |
| HUMANK_T27 (SEQ ID NO: 41) | 2272 | 2348 |
| HUMANK_T28 (SEQ ID NO: 42) | 2272 | 2348 |
| HUMANK_T35 (SEQ ID NO: 43) | 2272 | 2348 |

Segment cluster HUMANK_node_{—}99 (SEQ ID NO:298) according to the present invention can be found in the following transcript(s): HUMANK_T3 (SEQ ID NO:36), HUMANK_T13 (SEQ ID NO:37), HUMANK_T23 (SEQ ID NO:38), HUMANK_T24 (SEQ ID NO:39), HUMANK_T26 (SEQ ID NO:40), HUMANK_T27 (SEQ ID NO:41), HUMANK_T28 (SEQ ID NO:42) and HUMANK_T35 (SEQ ID NO:43). Table 37 below describes the starting and ending position of this segment on each transcript.

TABLE 37
Segment location on transcripts

| | Segment | Segment |
|---|---|---|
| Transcript name | starting position | ending position |
| HUMANK_T3 (SEQ ID NO: 36) | 2349 | 2355 |
| HUMANK_T13 (SEQ ID NO: 37) | 2349 | 2355 |
| HUMANK_T23 (SEQ ID NO: 38) | 2349 | 2355 |
| HUMANK_T24 (SEQ ID NO: 39) | 2349 | 2355 |
| HUMANK_T26 (SEQ ID NO: 40) | 2349 | 2355 |
| HUMANK_T27 (SEQ ID NO: 41) | 2349 | 2355 |
| HUMANK_T28 (SEQ ID NO: 42) | 2349 | 2355 |
| HUMANK_T35 (SEQ ID NO: 43) | 2349 | 2355 |

Segment cluster HUMANK_node_{—}102 (SEQ ID NO:299) according to the present invention is supported by 57 libraries. The number of libraries was determined as previously described. This segment can be found in the following transcript(s): HUMANK_T3 (SEQ ID NO:36), HUMANK_T13 (SEQ ID NO:37), HUMANK_T26 (SEQ ID NO:40), HUMANK_T27 (SEQ ID NO:41) and HUMANK_T28 (SEQ ID NO:42). Table 38 below describes the starting and ending position of this segment on each transcript.

TABLE 38
Segment location on transcripts

| | Segment | Segment |
|---|---|---|
| Transcript name | starting position | ending position |
| HUMANK_T3 (SEQ ID NO: 36) | 2356 | 2387 |
| HUMANK_T13 (SEQ ID NO: 37) | 2356 | 2387 |
| HUMANK_T26 (SEQ ID NO: 40) | 2356 | 2387 |
| HUMANK_T27 (SEQ ID NO: 41) | 2356 | 2387 |
| HUMANK_T28 (SEQ ID NO: 42) | 2356 | 2387 |

Segment cluster HUMANK_node_{—}103 (SEQ ID NO:300) according to the present invention can be found in the following transcript(s): HUMANK_T3 (SEQ ID NO:36), HUMANK_T13 (SEQ ID NO:37), HUMANK_T26 (SEQ ID NO:40), HUMANK_T27 (SEQ ID NO:41) and HUMANK_T28 (SEQ ID NO:42). Table 39 below describes the starting and ending position of this segment on each transcript.

TABLE 39
Segment location on transcripts

| | Segment | Segment |
|---|---|---|
| Transcript name | starting position | ending position |
| HUMANK_T3 (SEQ ID NO: 36) | 2388 | 2409 |
| HUMANK_T13 (SEQ ID NO: 37) | 2388 | 2409 |
| HUMANK_T26 (SEQ ID NO: 40) | 2388 | 2409 |
| HUMANK_T27 (SEQ ID NO: 41) | 2388 | 2409 |
| HUMANK_T28 (SEQ ID NO: 42) | 2388 | 2409 |

Segment cluster HUMANK_node_{—}104 (SEQ ID NO:301) according to the present invention can be found in the following transcript(s): HUMANK_T3 (SEQ ID NO:36), HUMANK_T13 (SEQ ID NO:37), HUMANK_T26 (SEQ ID NO:40), HUMANK_T27 (SEQ ID NO:41) and HUMANK_T28 (SEQ ID NO:42). Table 40 below describes the starting and ending position of this segment on each transcript.

TABLE 40
Segment location on transcripts

| | Segment | Segment |
|---|---|---|
| Transcript name | starting position | ending position |
| HUMANK_T3 (SEQ ID NO: 36) | 2410 | 2421 |
| HUMANK_T13 (SEQ ID NO: 37) | 2410 | 2421 |
| HUMANK_T26 (SEQ ID NO: 40) | 2410 | 2421 |
| HUMANK_T27 (SEQ ID NO: 41) | 2410 | 2421 |
| HUMANK_T28 (SEQ ID NO: 42) | 2410 | 2421 |

Segment cluster HUMANK_node_{—}105 (SEQ ID NO:302) according to the present invention is supported by 33 libraries. The number of libraries was determined as previously described. This segment can be found in the following transcript(s): HUMANK_T13 (SEQ ID NO:37), HUMANK_T26 (SEQ ID NO:40), HUMANK_T27 (SEQ ID NO:41) and HUMANK_T28 (SEQ ID NO:42). Table 41 below describes the starting and ending position of this segment on each transcript.

TABLE 41
Segment location on transcripts

| | Segment | Segment |
|---|---|---|
| Transcript name | starting position | ending position |
| HUMANK_T13 (SEQ ID NO: 37) | 2422 | 2463 |
| HUMANK_T26 (SEQ ID NO: 40) | 2422 | 2463 |
| HUMANK_T27 (SEQ ID NO: 41) | 2422 | 2463 |
| HUMANK_T28 (SEQ ID NO: 42) | 2422 | 2463 |

Segment cluster HUMANK_node_{—}106 (SEQ ID NO:303) according to the present invention is supported by 31 libraries. The number of libraries was determined as previously described. This segment can be found in the following transcript(s): HUMANK_T13 (SEQ ID NO:37) and HUMANK_T27 (SEQ ID NO:41). Table 42 below describes the starting and ending position of this segment on each transcript.

TABLE 42
Segment location on transcripts

| | Segment | Segment |
|---|---|---|
| Transcript name | starting position | ending position |
| HUMANK_T13 (SEQ ID NO: 37) | 2464 | 2496 |
| HUMANK_T27 (SEQ ID NO: 41) | 2464 | 2496 |

Segment cluster HUMANK_node_{—}112 (SEQ ID NO:304) according to the present invention is supported by 56 libraries. The number of libraries was determined as previously described. This segment can be found in the following transcript(s): HUMANK_T3 (SEQ ID NO:36), HUMANK_T13 (SEQ ID NO:37), HUMANK_T23 (SEQ ID NO:38), HUMANK T24 (SEQ ID NO:39), HUMANK T26 (SEQ ID NO:40), HUMANK_T27 (SEQ ID NO:41) and HUMANK_T28 (SEQ ID NO:42). Table 43 below describes the starting and ending position of this segment on each transcript.

TABLE 43
Segment location on transcripts

| | Segment | Segment |
|---|---|---|
| Transcript name | starting position | ending position |
| HUMANK_T3 (SEQ ID NO: 36) | 2557 | 2595 |
| HUMANK_T13 (SEQ ID NO: 37) | 2497 | 2535 |
| HUMANK_T23 (SEQ ID NO: 38) | 2356 | 2394 |
| HUMANK_T24 (SEQ ID NO: 39) | 2491 | 2529 |
| HUMANK_T26 (SEQ ID NO: 40) | 2599 | 2637 |
| HUMANK_T27 (SEQ ID NO: 41) | 2632 | 2670 |
| HUMANK_T28 (SEQ ID NO: 42) | 2464 | 2502 |

Segment cluster HUMANK_node_{—}114 (SEQ ID NO:305) according to the present invention is supported by 55 libraries. The number of libraries was determined as previously described. This segment can be found in the following transcript(s): HUMANK_T3 (SEQ ID NO:36), HUMANK_T13 (SEQ ID NO:37), HUMANK_T23 (SEQ ID NO:38), HUMANK_T24 (SEQ ID NO:39), HUMANK_T26 (SEQ ID NO:40), HUMANK_T27 (SEQ ID NO:41) and HUMANK_T28 (SEQ ID NO:42). Table 44 below describes the starting and ending position of this segment on each transcript.

TABLE 44
Segment location on transcripts

| | Segment | Segment |
|---|---|---|
| Transcript name | starting position | ending position |
| HUMANK_T3 (SEQ ID NO: 36) | 2736 | 2820 |
| HUMANK_T13 (SEQ ID NO: 37) | 2676 | 2760 |
| HUMANK_T23 (SEQ ID NO: 38) | 2535 | 2619 |
| HUMANK_T24 (SEQ ID NO: 39) | 2670 | 2754 |
| HUMANK_T26 (SEQ ID NO: 40) | 2778 | 2862 |
| HUMANK_T27 (SEQ ID NO: 41) | 2811 | 2895 |
| HUMANK_T28 (SEQ ID NO: 42) | 2643 | 2727 |

Segment cluster HUMANK_node_{—}116 (SEQ ID NO:306) according to the present invention can be found in the following transcript(s): HUMANK_T3 (SEQ ID NO:36). Table 45 below describes the starting and ending position of this segment on each transcript.

TABLE 45
Segment location on transcripts

| | Segment | Segment |
|---|---|---|
| Transcript name | starting position | ending position |
| HUMANK_T3 (SEQ ID NO: 36) | 3235 | 3248 |

### Variant Protein Alignment to the Previously Known Protein:

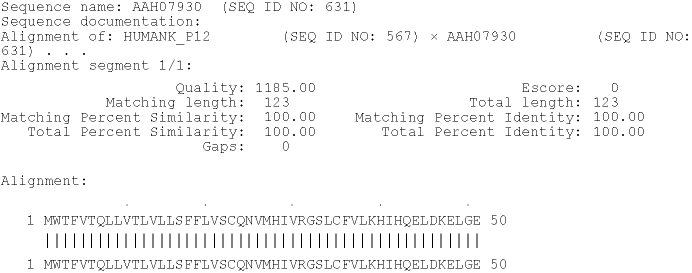

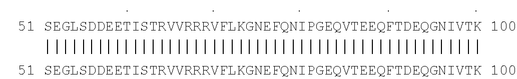

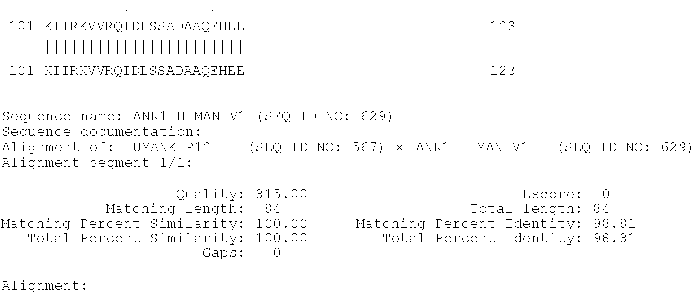

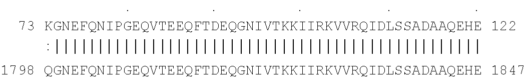

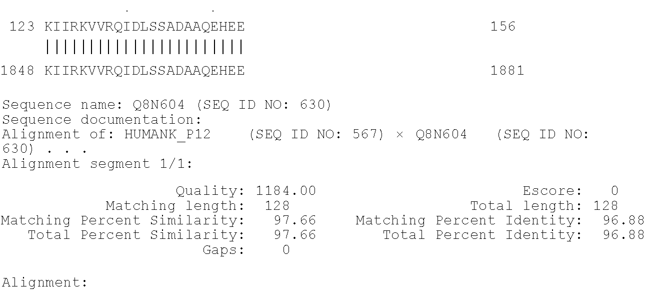

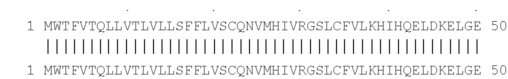

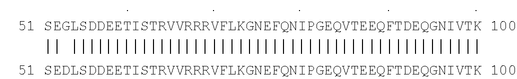

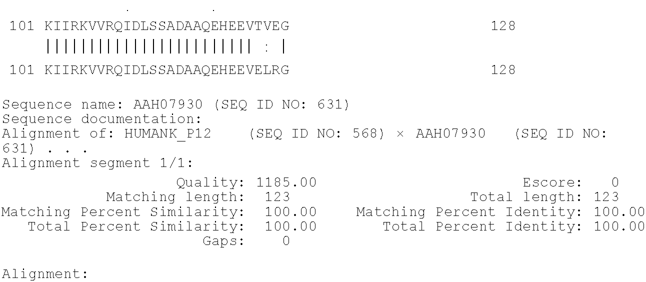

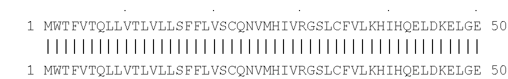

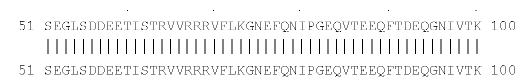

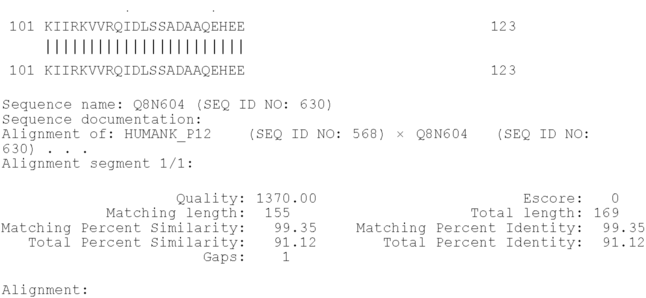

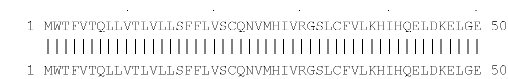

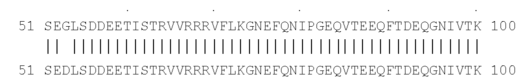

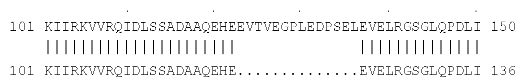

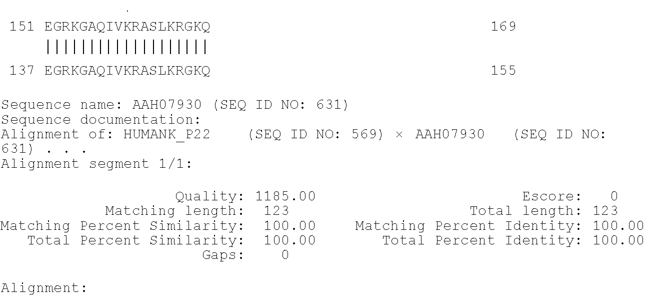

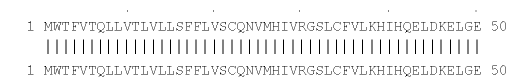

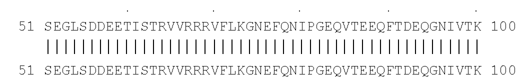

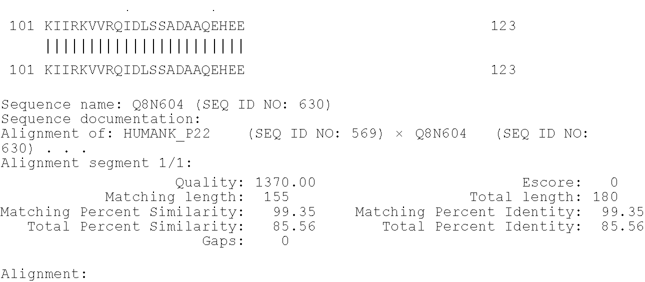

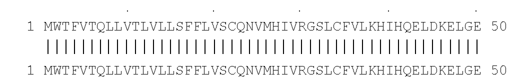

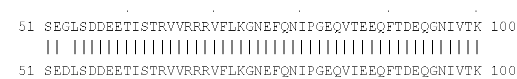

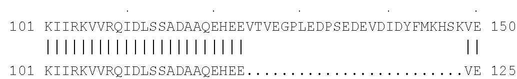

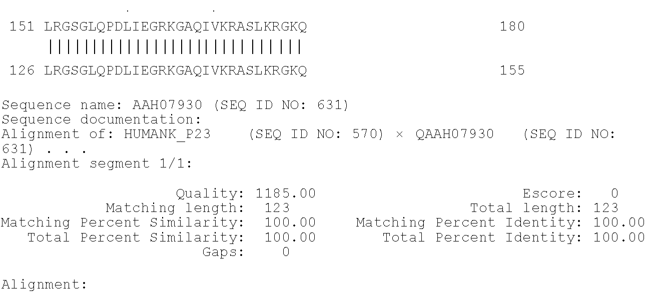

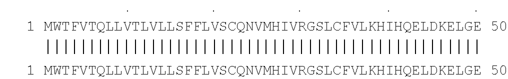

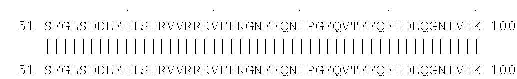

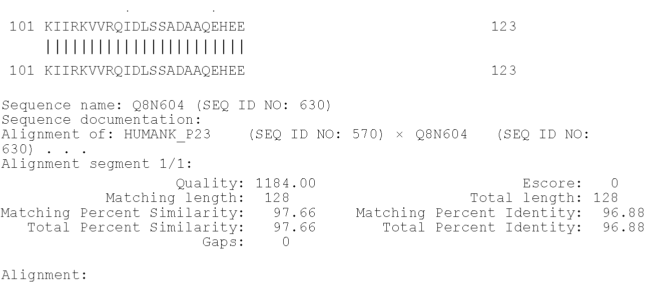

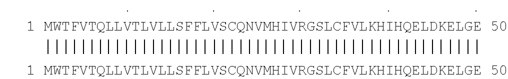

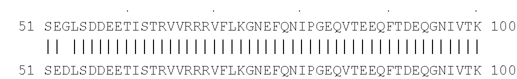

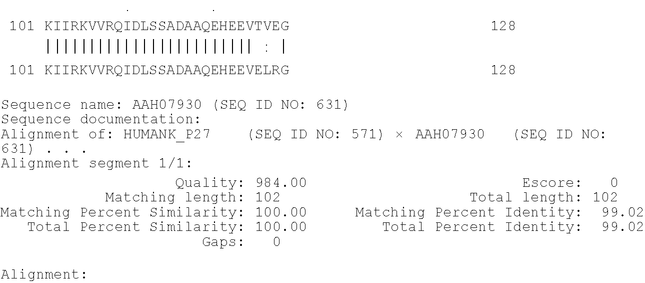

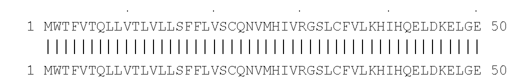

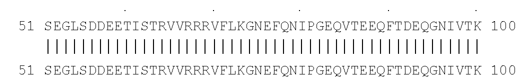

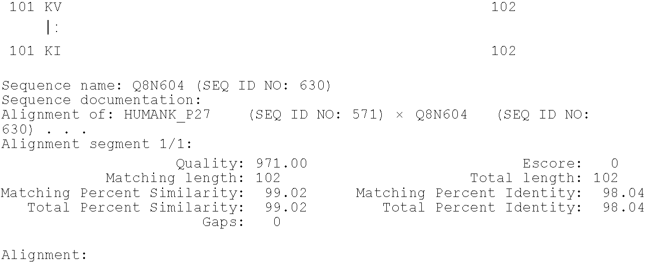

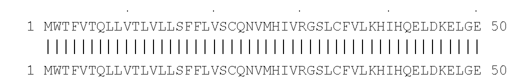

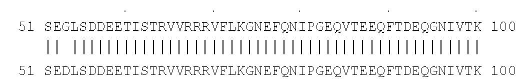

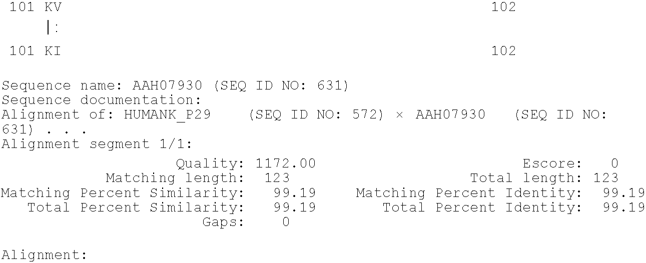

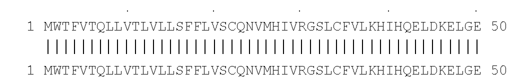

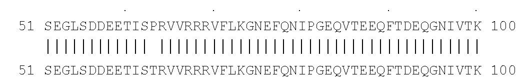

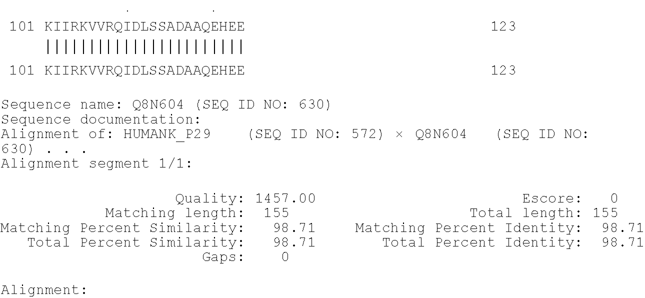

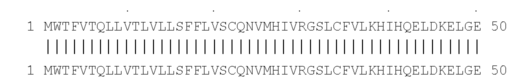

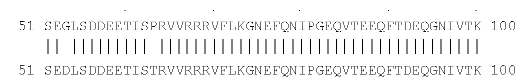

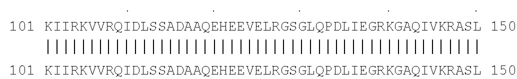

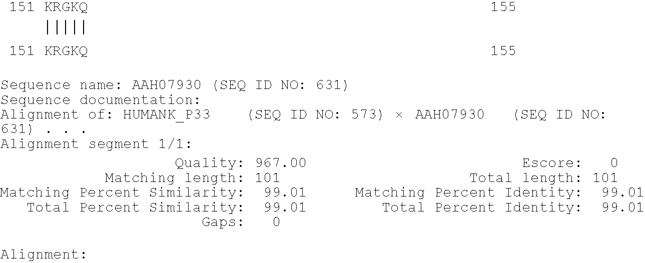

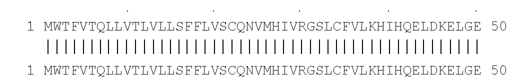

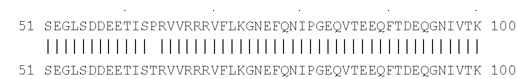

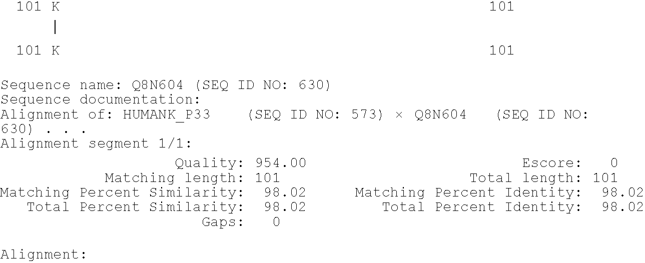

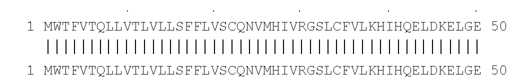

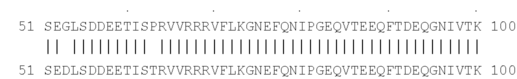

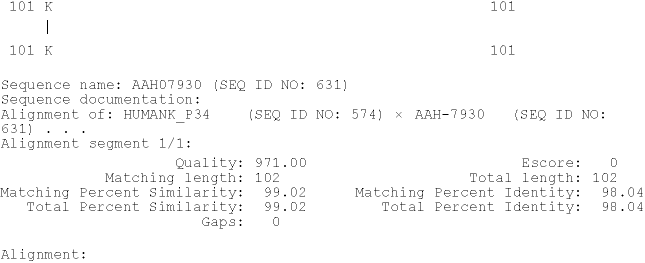

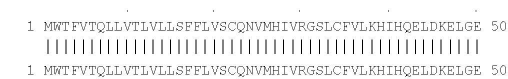

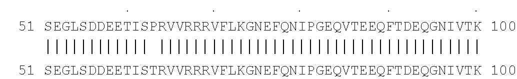

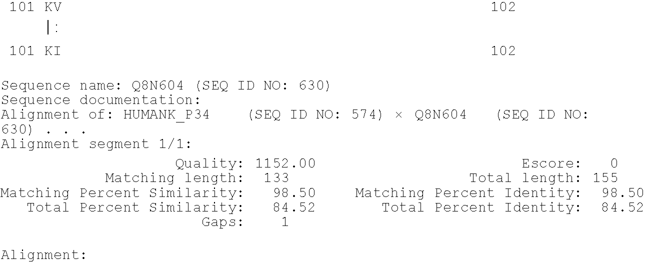

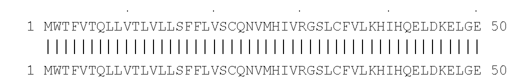

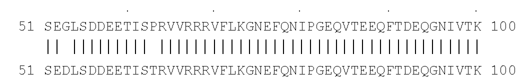

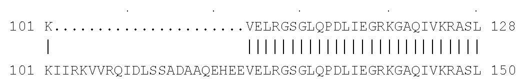

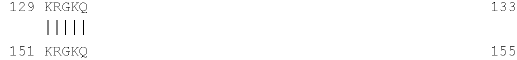

### Description for Cluster Z39819

Cluster Z39819 features 1 transcript(s) and 10 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| Z39819_PEA_1_T2 | 44 |

TABLE 2
Segments of interest

| Segment Name | SEQ ID NO: |
|---|---|
| Z39819_PEA_1_node_2 | 307 |
| Z39819_PEA_1_node_6 | 308 |
| Z39819_PEA_1_node_10 | 309 |
| Z39819_PEA_1_node_14 | 310 |
| Z39819_PEA_1_node_16 | 311 |
| Z39819_PEA_1_node_21 | 312 |
| Z39819_PEA_1_node_3 | 313 |
| Z39819_PEA_1_node_8 | 314 |
| Z39819_PEA_1_node_12 | 315 |
| Z39819_PEA_1_node_19 | 316 |

TABLE 3
Proteins of interest

| Protein Name | SEQ ID NO: | Corresponding Transcript(s) |
|---|---|---|
| Z39819_PEA_1_P6 | 575 | Z39819_PEA_1_T2 (SEQ ID |
| | | NO: 44) |

These sequences are variants of the known protein GDNF family receptor alpha 2 precursor (SwissProt accession identifier GFR2_HUMAN; known also according to the synonyms GFR-alpha 2; Neurturin receptor alpha; NTNR-alpha; NRTNR-alpha; TGF-beta related neurotrophic factor receptor 2; GDNF receptor beta; GDNFR-beta; RET ligand 2), SEQ ID NO:632, referred to herein as the previously known protein.

Protein GDNF family receptor alpha 2 precursor (SEQ ID NO:632) is known or believed to have the following function(s): Receptor for neurturin. Mediates the NRTN-induced autophosphorylation and activation of the RET receptor. Also able to mediate GDNF signaling through the RET tyrosine kinase receptor. The sequence for protein GDNF family receptor alpha 2 precursor is given at the end of the application, as “GDNF family receptor alpha 2 precursor amino acid sequence”. Known polymorphisms for this sequence are as shown in Table 4.

TABLE 4
Amino acid mutations for Known Protein

| SNP position(s) on amino | |
|---|---|
| acid sequence | Comment |
| 6 | V -> A |
| 462 | Q -> L |

Protein GDNF family receptor alpha 2 precursor (SEQ ID NO:632) localization is believed to be attached to the membrane by a GPI-anchor (By similarity).

The following GO Annotation(s) apply to the previously known protein. The following annotation(s) were found: transmembrane receptor protein tyrosine kinase signaling pathway, which are annotation(s) related to Biological Process; and receptor; glial cell line-derived neurotrophic factor receptor, which are annotation(s) related to Molecular Function.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

As noted above, cluster Z39819 features 1 transcript(s), which were listed in Table 1 above. These transcript(s) encode for protein(s) which are variant(s) of protein GDNF family receptor alpha 2 precursor (SEQ ID NO:632). A description of each variant protein according to the present invention is now provided.

Variant protein Z39819_PEA_{—}1_P6 (SEQ ID NO:575) according to the present invention has an amino acid sequence as given at the end of the application; it is encoded by transcript(s) Z39819_PEA_{—}1_T2 (SEQ ID NO:44). An alignment is given to the known protein (GDNF family receptor alpha 2 precursor (SEQ ID NO:632)) at the end of the application. One or more alignments to one or more previously published protein sequences are given at the end of the application. A brief description of the relationship of the variant protein according to the present invention to each such aligned protein is as follows:

Comparison Report Between Z39819_PEA_{—}1_P6 (SEQ ID NO:575) and GFR2_HUMAN (SEQ ID NO:632):

1. An isolated chimeric polypeptide encoding for Z39819_PEA_{—}1_P6 (SEQ ID NO:575), comprising a first amino acid sequence being at least 90% homologous to MILANVFCLFFFL corresponding to amino acids 1-13 of GFR2_HUMAN (SEQ ID NO:632), which also corresponds to amino acids 1-13 of Z39819_PEA_{—}1_P6 (SEQ ID NO:575), and a second amino acid sequence being at least 70%, optionally at least 80%, preferably at least 85%, more preferably at least 90% and most preferably at least 95% homologous to a polypeptide having the sequence GPRAPRLAPPSGLCPGQ (SEQ ID NO:1520) corresponding to amino acids 14-30 of Z39819_PEA_{—}1_P6 (SEQ ID NO:575), wherein said first amino acid sequence and second amino acid sequence are contiguous and in a sequential order.

2. An isolated polypeptide encoding for a tail of Z39819_PEA_{—}1_P6 (SEQ ID NO:575), comprising a polypeptide being at least 70%, optionally at least about 80%, preferably at least about 85%, more preferably at least about 90% and most preferably at least about 95% homologous to the sequence GPRAPRLAPPSGLCPGQ (SEQ ID NO:1520) in Z39819_PEA_{—}1_P6 (SEQ ID NO:575).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

The glycosylation sites of variant protein Z39819_PEA_{—}1_P6 (SEQ ID NO:575), as compared to the known protein GDNF family receptor alpha 2 precursor (SEQ ID NO:632), are described in Table 5 (given according to their position(s) on the amino acid sequence in the first column; the second column indicates whether the glycosylation site is present in the variant protein; and the last column indicates whether the position is different on the variant protein).

TABLE 5
Glycosylation site(s)

| Position(s) on known amino acid | |
|---|---|
| sequence | Present in variant protein? |
| 413 | No |
| 357 | No |
| 52 | No |

Variant protein Z39819_PEA_{—}1_P6 (SEQ ID NO:575) is encoded by the following transcript(s): Z39819_PEA_{—}1_T2 (SEQ ID NO:44), for which the sequence(s) is/are given at the end of the application. The coding portion of transcript Z39819_PEA_{—}1_T2 (SEQ ID NO:44) is shown in bold; this coding portion starts at position 715 and ends at position 804. The transcript also has the following SNPs as listed in Table 6 (given according to their position on the nucleotide sequence, with the alternative nucleic acid listed; the last column indicates whether the SNP is known or not; the presence of known SNPs in variant protein Z39819_PEA_{—}1_P6 (SEQ ID NO:575) sequence provides support for the deduced sequence of this variant protein according to the present invention).

TABLE 6
Nucleic acid SNPs

| SNP position on nucleotide | Alternative | Previously |
|---|---|---|
| sequence | nucleic acid | known SNP? |
| 633 | C -> G | No |
| 1088 | T -> G | No |
| 1114 | G -> C | No |
| 1120 | G -> C | No |
| 1372 | C -> | No |
| 1380 | C -> | No |
| 1872 | C -> T | No |
| 2058 | A -> T | Yes |
| 2090 | G -> T | Yes |
| 2161 | T -> C | No |
| 2165 | A -> | No |
| 2165 | A -> C | No |
| 2256 | C -> A | No |

As noted above, cluster Z39819 features 10 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster Z39819_PEA_{—}1_node_{—}2 (SEQ ID NO:307) according to the present invention is supported by 17 libraries. The number of libraries was determined as previously described. This segment can be found in the following transcript(s): Z39819_PEA_{—}1_T2 (SEQ ID NO:44). Table 7 below describes the starting and ending position of this segment on each transcript.

TABLE 7
Segment location on transcripts

| | Segment | Segment |
|---|---|---|
| Transcript name | starting position | ending position |
| Z39819_PEA_1_T2 | 1 | 679 |
| (SEQ ID NO: 44) | | |

Segment cluster Z39819_PEA_{—}1_node_{—}6 (SEQ ID NO:308) according to the present invention is supported by 17 libraries. The number of libraries was determined as previously described. This segment can be found in the following transcript(s): Z39819_PEA_{—}1_T2 (SEQ ID NO:44). Table 8 below describes the starting and ending position of this segment on each transcript.

TABLE 8
Segment location on transcripts

| | Segment | Segment |
|---|---|---|
| Transcript name | starting position | ending position |
| Z39819_PEA_1_T2 | 755 | 1028 |
| (SEQ ID NO: 44) | | |

Segment cluster Z39819_PEA_{—}1_node_{—}10 (SEQ ID NO:309) according to the present invention is supported by 23 libraries. The number of libraries was determined as previously described. This segment can be found in the following transcript(s): Z39819_PEA_{—}1_T2 (SEQ ID NO:44). Table 9 below describes the starting and ending position of this segment on each transcript.

TABLE 9
Segment location on transcripts

| | Segment | Segment |
|---|---|---|
| Transcript name | starting position | ending position |
| Z39819_PEA_1_T2 | 1113 | 1467 |
| (SEQ ID NO: 44) | | |

Segment cluster Z39819_PEA_{—}1_node_{—}14 (SEQ ID NO:310) according to the present invention is supported by 25 libraries. The number of libraries was determined as previously described. This segment can be found in the following transcript(s): Z39819_PEA_{—}1_T2 (SEQ ID NO:44). Table 10 below describes the starting and ending position of this segment on each transcript.

TABLE 10
Segment location on transcripts

| | Segment | Segment |
|---|---|---|
| Transcript name | starting position | ending position |
| Z39819_PEA_1_T2 | 1578 | 1718 |
| (SEQ ID NO: 44) | | |

Segment cluster Z39819_PEA_{—}1_node_{—}16 (SEQ ID NO:311) according to the present invention is supported by 26 libraries. The number of libraries was determined as previously described. This segment can be found in the following transcript(s): Z39819_PEA_{—}1_T2 (SEQ ID NO:44). Table 11 below describes the starting and ending position of this segment on each transcript.

TABLE 11
Segment location on transcripts

| | Segment | Segment |
|---|---|---|
| Transcript name | starting position | ending position |
| Z39819_PEA_1_T2 | 1719 | 1891 |
| (SEQ ID NO: 44) | | |

Segment cluster Z39819_PEA_{—}1_node_{—}21 (SEQ ID NO:312) according to the present invention is supported by 61 libraries. The number of libraries was determined as previously described. This segment can be found in the following transcript(s): Z39819_PEA_{—}1_T2 (SEQ ID NO:44). Table 12 below describes the starting and ending position of this segment on each transcript.

TABLE 12
Segment location on transcripts

| | Segment | Segment |
|---|---|---|
| Transcript name | starting position | ending position |
| Z39819_PEA_1_T2 | 1946 | 3332 |
| (SEQ ID NO: 44) | | |

According to an optional embodiment of the present invention, short segments related to the above cluster are also provided. These segments are up to about 120 bp in length, and so are included in a separate description.

Segment cluster Z39819_PEA_{—}1_node_{—}3 (SEQ ID NO:313) according to the present invention is supported by 14 libraries. The number of libraries was determined as previously described. This segment can be found in the following transcript(s): Z39819_PEA_{—}1_T2 (SEQ ID NO:44). Table 13 below describes the starting and ending position of this segment on each transcript.

TABLE 13
Segment location on transcripts

| | Segment | Segment |
|---|---|---|
| Transcript name | starting position | ending position |
| Z39819_PEA_1_T2 | 680 | 754 |
| (SEQ ID NO: 44) | | |

Segment cluster Z39819_PEA_{—}1_node_{—}8 (SEQ ID NO:314) according to the present invention is supported by 14 libraries. The number of libraries was determined as previously described. This segment can be found in the following transcript(s): Z39819_PEA_{—}1_T2 (SEQ ID NO:44). Table 14 below describes the starting and ending position of this segment on each transcript.

TABLE 14
Segment location on transcripts

| | Segment | |
|---|---|---|
| | starting | Segment |
| Transcript name | position | ending position |
| Z39819_PEA_1_T2 (SEQ ID NO: 44) | 1029 | 1112 |

Segment cluster Z39819_PEA_{—}1_node_{—}12 (SEQ ID NO:315) according to the present invention is supported by 20 libraries. The number of libraries was determined as previously described. This segment can be found in the following transcript(s): Z39819_PEA_{—}1_T2 (SEQ ID NO:44). Table 15 below describes the starting and ending position of this segment on each transcript.

TABLE 15
Segment location on transcripts

| | Segment | |
|---|---|---|
| | starting | Segment |
| Transcript name | position | ending position |
| Z39819_PEA_1_T2 (SEQ ID NO: 44) | 1468 | 1577 |

Segment cluster Z39819_PEA_{—}1_node_{—}19 (SEQ ID NO:316) according to the present invention is supported by 27 libraries. The number of libraries was determined as previously described. This segment can be found in the following transcript(s): Z39819_PEA_{—}1_T2 (SEQ ID NO:44). Table 16 below describes the starting and ending position of this segment on each transcript.

TABLE 16
Segment location on transcripts

| | Segment | |
|---|---|---|
| | starting | Segment |
| Transcript name | position | ending position |
| Z39819_PEA_1_T2 (SEQ ID NO: 44) | 1892 | 1945 |

### Variant Protein Alignment to the Previously Known Protein:

Sequence name: GFR2_HUMAN (SEQ ID NO: 632)
Sequence documentation:
Alignment of: Z39819_PEA_1 PG (SEQ ID NO: 575) × GFR2_HUMAN (SEQ ID
NO: 632) . .
Alignment segment 1/1:

| | | | |
|---|---|---|---|
| Quality: | 146.00 | Escore: | 0 |
| Matching length: | 26 | Total length: | 26 |
| Matching Percent Similarity: | 69.23 | Matching Percent Identity: | 69.23 |
| Total Percent Similarity: | 69.23 | Total Percent Identity: | 69.23 |
| Gaps: | 0 | | |

Alignment:

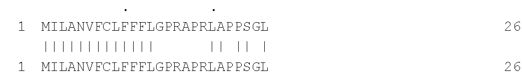

### Description for Cluster HUMCA1XIA

Cluster HUMCA1XIA features 4 transcript(s) and 46 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| HUMCA1XIA_T16 | 45 |
| HUMCA1XIA_T17 | 46 |
| HUMCA1XIA_T19 | 47 |
| HUMCA1XIA_T20 | 48 |

TABLE 2
Segments of interest

| Segment Name | SEQ ID NO: |
|---|---|
| HUMCA1XIA_node_0 | 317 |
| HUMCA1XIA_node_2 | 318 |
| HUMCA1XIA_node_4 | 319 |
| HUMCA1XIA_node_6 | 320 |
| HUMCA1XIA_node_8 | 321 |
| HUMCA1XIA_node_9 | 322 |
| HUMCA1XIA_node_18 | 323 |
| HUMCA1XIA_node_54 | 324 |
| HUMCA1XIA_node_55 | 325 |
| HUMCA1XIA_node_92 | 326 |
| HUMCA1XIA_node_11 | 327 |
| HUMCA1XIA_node_15 | 328 |
| HUMCA1XIA_node_19 | 329 |
| HUMCA1XIA_node_21 | 330 |
| HUMCA1XIA_node_23 | 331 |
| HUMCA1XIA_node_25 | 332 |
| HUMCA1XIA_node_27 | 333 |
| HUMCA1XIA_node_29 | 334 |
| HUMCA1XIA_node_31 | 335 |
| HUMCA1XIA_node_33 | 336 |
| HUMCA1XIA_node_35 | 337 |
| HUMCA1XIA_node_37 | 338 |
| HUMCA1XIA_node_39 | 339 |
| HUMCA1XIA_node_41 | 340 |
| HUMCA1XIA_node_43 | 341 |
| HUMCA1XIA_node_45 | 342 |
| HUMCA1XIA_node_47 | 343 |
| HUMCA1XIA_node_49 | 344 |
| HUMCA1XIA_node_51 | 345 |
| HUMCA1XIA_node_57 | 346 |
| HUMCA1XIA_node_59 | 347 |
| HUMCA1XIA_node_62 | 348 |
| HUMCA1XIA_node_64 | 349 |
| HUMCA1XIA_node_66 | 350 |
| HUMCA1XIA_node_68 | 351 |
| HUMCA1XIA_node_70 | 352 |
| HUMCA1XIA_node_72 | 353 |
| HUMCA1XIA_node_74 | 354 |
| HUMCA1XIA_node_76 | 355 |
| HUMCA1XIA_node_78 | 356 |
| HUMCA1XIA_node_81 | 357 |
| HUMCA1XIA_node_83 | 358 |
| HUMCA1XIA_node_85 | 359 |
| HUMCA1XIA_node_87 | 360 |
| HUMCA1XIA_node_89 | 361 |
| HUMCA1XIA_node_91 | 362 |

TABLE 3
Proteins of interest

| Protein Name | SEQ ID NO: | Corresponding Transcript(s) |
|---|---|---|
| HUMCA1XIA_P14 | 576 | HUMCA1XIA_T16 (SEQ ID |
| | | NO: 45) |
| HUMCA1XIA_P15 | 577 | HUMCA1XIA_T17 (SEQ ID |
| | | NO: 46) |
| HUMCA1XIA_P16 | 578 | HUMCA1XIA_T19 (SEQ ID |
| | | NO: 47) |
| HUMCA1XIA_P17 | 579 | HUMCA1XIA_T20 (SEQ ID |
| | | NO: 48) |

These sequences are variants of the known protein Collagen alpha 1 (SwissProt accession identifier CA1B_HUMAN; known also according to the synonyms XI), SEQ ID NO: 633, referred to herein as the previously known protein.

Protein Collagen alpha 1 (SEQ ID NO:633) is known or believed to have the following function(s): May play an important role in fibrillogenesis by controlling lateral growth of collagen II fibrils. The sequence for protein Collagen alpha 1 is given at the end of the application, as “Collagen alpha 1 amino acid sequence”. Known polymorphisms for this sequence are as shown in Table 4.

TABLE 4
Amino acid mutations for Known Protein

| SNP position(s) on | |
|---|---|
| amino acid sequence | Comment |
| 625 | G -> V (in STL2). /FTId = VAR_013583. |
| 676 | G -> R (in STL2; overlapping phenotype |
| | with Marshall syndrome). /FTId = VAR_013584. |
| 921-926 | Missing (in STL2; overlapping phenotype |
| | with Marshall syndrome). /FTId = VAR_013585. |
| 1313-1315 | Missing (in STL2; overlapping phenotype |
| | with Marshall syndrome). /FTId = VAR_013586. |
| 1516 | G -> V (in STL2; overlapping phenotype |
| | with Marshall syndrome). /FTId = VAR_013587. |
| 941-944 | KDGL -> RMGC |
| 986 | Y -> H |
| 1074 | R -> P |
| 1142 | G -> D |
| 1218 | M -> W |
| 1758 | T -> A |
| 1786 | S -> N |

The following GO Annotation(s) apply to the previously known protein. The following annotation(s) were found: cartilage condensation; vision; hearing; cell-cell adhesion; extracellular matrix organization and biogenesis, which are annotation(s) related to Biological Process; extracellular matrix structural protein; extracellular matrix protein, adhesive, which are annotation(s) related to Molecular Function; and extracellular matrix; collagen; collagen type XI, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster HUMCA1XIA can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

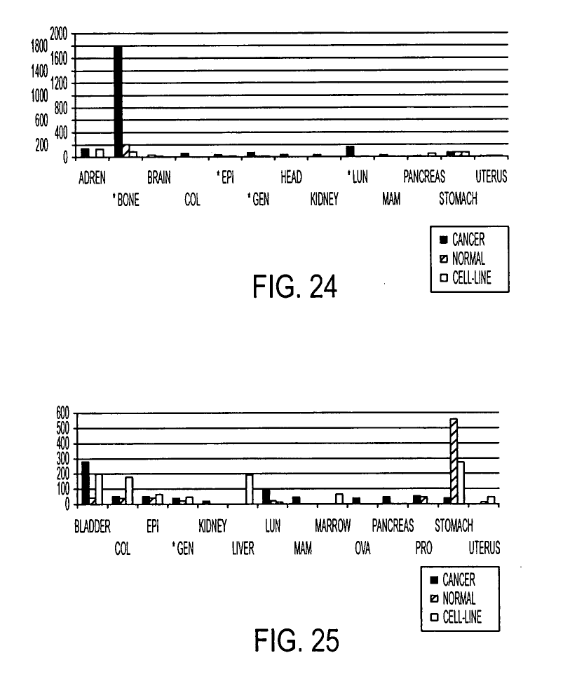

Overall, the following results were obtained as shown with regard to the histograms in FIG. 24 and Table 5. This cluster is overexpressed (at least at a minimum level) in the following pathological conditions: bone malignant tumors, epithelial malignant tumors, a mixture of malignant tumors from different tissues and lung malignant tumors.

TABLE 5
Normal tissue distribution

| Name of Tissue | Number |
|---|---|
| adrenal | 0 |
| Bone | 207 |
| brain | 13 |
| colon | 0 |
| epithelial | 11 |
| general | 11 |
| Head and neck | 0 |
| kidney | 0 |
| Lung | 0 |
| breast | 8 |
| pancreas | 0 |
| stomach | 73 |
| uterus | 9 |

TABLE 6
P values and ratios for expression in cancerous tissue

| Name of Tissue | P1 | P2 | SP1 | R3 | SP2 | R4 |
|---|---|---|---|---|---|---|
| adrenal | 4.2e−01 | 1.9e−01 | 9.6e−02 | 3.4 | 8.2e−02 | 3.6 |
| bone | 2.4e−01 | 6.3e−01 | 7.7e−10 | 4.3 | 5.3e−03 | 1.6 |
| brain | 5.0e−01 | 6.9e−01 | 1.8e−01 | 2.1 | 4.2e−01 | 1.3 |
| colon | 1.3e−02 | 2.9e−02 | 2.4e−01 | 3.0 | 3.5e−01 | 2.4 |
| epithelial | 3.9e−04 | 3.2e−03 | 1.3e−03 | 2.3 | 1.8e−02 | 1.7 |
| general | 5.6e−05 | 1.6e−03 | 9.5e−17 | 4.5 | 1.1e−09 | 2.8 |
| head and neck | 1.2e−01 | 2.1e−01 | 1 | 1.3 | 1 | 1.1 |
| kidney | 6.5e−01 | 7.2e−01 | 3.4e−01 | 2.4 | 4.9e−01 | 1.9 |
| lung | 5.3e−02 | 9.1e−02 | 5.5e−05 | 7.3 | 5.0e−03 | 4.0 |
| breast | 4.3e−01 | 5.6e−01 | 6.9e−01 | 1.4 | 8.2e−01 | 1.1 |
| pancreas | 3.3e−01 | 1.8e−01 | 4.2e−01 | 2.4 | 1.5e−01 | 3.7 |
| stomach | 5.0e−01 | 6.1e−01 | 6.9e−01 | 1.0 | 6.7e−01 | 0.8 |
| uterus | 7.1e−01 | 7.0e−01 | 6.6e−01 | 1.1 | 6.4e−01 | 1.1 |

As noted above, cluster HUMCA1XIA features 4 transcript(s), which were listed in Table 1 above. These transcript(s) encode for protein(s) which are variant(s) of protein Collagen alpha 1 (SEQ ID NO:633). A description of each variant protein according to the present invention is now provided.

Variant protein HUMCA1XIA_P14 (SEQ ID NO:576) according to the present invention has an amino acid sequence as given at the end of the application; it is encoded by transcript(s) HUMCA1XIA_T16 (SEQ ID NO:45). An alignment is given to the known protein (Collagen alpha 1 (SEQ ID NO:633)) at the end of the application. One or more alignments to one or more previously published protein sequences are given at the end of the application. A brief description of the relationship of the variant protein according to the present invention to each such aligned protein is as follows:

Comparison Report Between HUMCA1XIA_P14 (SEQ ID NO:576) and CA1B_HUMAN_V5 (SEQ ID NO 634):

1. An isolated chimeric polypeptide encoding for HUMCA1XIA_P14 (SEQ ID NO:576), comprising a first amino acid sequence being at least 90% homologous to MEPWSSRWKTKRWLWDFTVTTLALTFLFQAREVRGAAPVDVLKALDFHNSPEGISKTTGFCTNRKNSKG SDTAYRVSKQAQLSAPTKQLFPGGTFPEDFSILFTVKPKKGIQSFLLSIYNEHGIQQIGVEVGRSPVFLFEDH TGKPAPEDYPLFRTVNIADGKWHRVAISVEKKTVTMIVDCKKKTTKPLDRSERAIVDTNGITVFGTRILDE EVFEGDIQQFLITGDPKAAYDYCEHYSPDCDSSAPKAAQAQEPQIDEYAPEDIIEYDYEYGEAEYKEAESVT EGPTVTEETIAQTEANIVDDFQEYNYGTMESYQTEAPRHVSGTNEPNPVEEIFTEEYLTGEDYDSQRKNSE DTLYENKEIDGRDSDLLVDGDLGEYDFYEYKEYEDKPTSPPNEEFGPGVPAETDITETSINGHGAYGEKGQ KGEPAVVEPGMLVEGPPGPAGPAGIMGPPGLQGPTGPPGDPGDRGPPGRPGLPGADGLPGPPGTMLMLPF RYGGDGSKGPTISAQEAQAQAILQQARIALRGPPGPMGLTGRPGPVGGPGSSGAKGESGDPGPQGPRGVQ GPPGPTGKPGKRGRPGADGGRGMPGEPGAKGDRGFDGLPGLPGDKGHRGERGPQGPPGPPGDDGMRGE DGEIGPRGLPGEAGPRGLLGPRGTPGAPGQPGMAGVDGPPGPKGNMGPQGEPGPPGQQGNPGPQGLPGPQ GPIGPPGEKGPQGKPGLAGLPGADGPPGHPGKEGQSGEKGALGPPGPQGPIGYPGPRGVKGADGVRGLKG SKGEKGEDGFPGFKGDMGLKGDRGEVGQIGPRGEDGPEGPKGRAGPTGDPGPSGQAGEKGKLGVPGLPG YPGRQGPKGSTGFPGFPGANGEKGARGVAGKPGPRGQRGPTGPRGSRGARGPTGKPGPKGTSGGDGPPGP PGERGPQGPQGPVGFPGPKGPPGPPGKDGLPGHPGQRGETGFQGKTGPPGPGGVVGPQGPTGETGPIGERG HPGPPGPPGEQGLPGAAGKEGAKGDPGPQGISGKDGPAGLRGFPGERGLPGAQGAPGLKGGEGPQGPPGP V corresponding to amino acids 1-1056 of CA1B_HUMAN_V5 (SEQ ID NO:634), which also corresponds to amino acids 1-1056 of HUMCA1XIA_P14 (SEQ ID NO:576), and a second amino acid sequence being at least 70%, optionally at least 80%, preferably at least 85%, more preferably at least 90% and most preferably at least 95% homologous to a polypeptide having the sequence VSMMIINSQTIMVVNYSSSFITLML (SEQ ID NO:1521) corresponding to amino acids 1057-1081 of HUMCA1XIA_P14 (SEQ ID NO:576), wherein said first amino acid sequence and second amino acid sequence are contiguous and in a sequential order.

2. An isolated polypeptide encoding for a tail of HUMCA1XIA_P14 (SEQ ID NO:576), comprising a polypeptide being at least 70%, optionally at least about 80%, preferably at least about 85%, more preferably at least about 90% and most preferably at least about 95% homologous to the sequence VSMMIINSQTIMVVNYSSSFITLML (SEQ ID NO:1521) in HUMCA1XIA_P14 (SEQ ID NO:576).

It should be noted that the known protein sequence (CA1B_HUMAN (SEQ ID NO:633)) has one or more changes than the sequence given at the end of the application and named as being the amino acid sequence for CA1B_HUMAN_V5 (SEQ ID NO:634). These changes were previously known to occur and are listed in the table below.

TABLE 7
Changes to CA1B_HUMAN_V5(SEQ ID NO: 634)

| SNP position(s) on amino | |
|---|---|
| acid sequence | Type of change |
| 987 | conflict |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMCA1XIA_P14 (SEQ ID NO:576) also has the following non-silent SNPs (Single Nucleotide Polymorphisms) as listed in Table 8, (given according to their position(s) on the amino acid sequence, with the alternative amino acid(s) listed; the last column indicates whether the SNP is known or not; the presence of known SNPs in variant protein HUMCA1XIA_P14 (SEQ ID NO:576) sequence provides support for the deduced sequence of this variant protein according to the present invention).

TABLE 8
Amino acid mutations

| SNP position(s) on amino | Alternative | Previously |
|---|---|---|
| acid sequence | amino acid(s) | known SNP? |
| 8 | W -> G | Yes |
| 46 | D -> E | Yes |
| 559 | G -> S | Yes |
| 832 | G -> * | Yes |
| 986 | H -> Y | Yes |
| 1061 | I -> M | Yes |
| 1070 | V -> A | Yes |

Variant protein HUMCA1XIA_P14 (SEQ ID NO:576) is encoded by the following transcript(s): HUMCA1XIA_T16 (SEQ ID NO:45), for which the sequence(s) is/are given at the end of the application. The coding portion of transcript HUMCA1XIA_T16 (SEQ ID NO:45) is shown in bold; this coding portion starts at position 319 and ends at position 3561. The transcript also has the following SNPs as listed in Table 9 (given according to their position on the nucleotide sequence, with the alternative nucleic acid listed; the last column indicates whether the SNP is known or not; the presence of known SNPs in variant protein HUMCA1XIA_P14 (SEQ ID NO:576) sequence provides support for the deduced sequence of this variant protein according to the present invention).

TABLE 9
Nucleic acid SNPs

| SNP position on | Alternative | Previously |
|---|---|---|
| nucleotide sequence | nucleic acid | known SNP? |
| 157 | A -> G | No |
| 241 | T -> A | Yes |
| 340 | T -> G | Yes |
| 456 | T -> G | Yes |
| 1993 | G -> A | Yes |
| 2812 | G -> T | Yes |
| 3274 | C -> T | Yes |
| 3282 | C -> T | Yes |
| 3501 | A -> G | Yes |
| 3527 | T -> C | Yes |

Variant protein HUMCA1XIA_P15 (SEQ ID NO:577) according to the present invention has an amino acid sequence as given at the end of the application; it is encoded by transcript(s) HUMCA1XIA_T17 (SEQ ID NO:46). An alignment is given to the known protein (Collagen alpha 1 (SEQ ID NO:633)) at the end of the application. One or more alignments to one or more previously published protein sequences are given at the end of the application. A brief description of the relationship of the variant protein according to the present invention to each such aligned protein is as follows:

Comparison Report Between HUMCA1XIA_P15 (SEQ ID NO:577) and CA1B_HUMAN (SEQ ID NO:633):

1. An isolated chimeric polypeptide encoding for HUMCA1XIA_P15 (SEQ ID NO:577), comprising a first amino acid sequence being at least 90% homologous to MEPWSSRWKTKRWLWDFTVTTLALTFLFQAREVRGAAPVDVLKALDFHNSPEGISKTTGFCTNRKNSKG SDTAYRVSKQAQLSAPTKQLFPGGTFPEDFSILFTVKPKKGIQSFLLSIYNEHGIQQIGVEVGRSPVFLFEDH TGKPAPEDYPLFRTVNIADGKWHRVAISVEKKTVTMIVDCKKKTTKPLDRSERAIVDTNGITVFGTRILDE EVFEGDIQQFLITGDPKAAYDYCEHYSPDCDSSAPKAAQAQEPQIDEYAPEDIIEYDYEYGEAEYKEAESVT EGPTVTEETIAQTEANIVDDFQEYNYGTMESYQTEAPRHVSGTNEPNPVEEIFTEEYLTGEDYDSQRKNSE DTLYENKEIDGRDSDLLVDGDLGEYDFYEYKEYEDKPTSPPNEEFGPGVPAETDITETSINGHGAYGEKGQ KGEPAVVEPGMLVEGPPGPAGPAGIMGPPGLQGPTGPPGDPGDRGPPGRPGLPGADGLPGPPGTMLMLPF RYGGDGSKGPTISAQEAQAQAILQQARIALRGPPGPMGLTGRPGPVGGPGSSGAKGESGDPGPQGPRGVQ GPPGPTGKPGKRGRPGADGGRGMPGEPGAKGDRGFDGLPGLPGDKGHRGERGPQGPPGPPGDDGMRGE DGEIGPRGLPGEAGPRGLLGPRGTPGAPGQPGMAGVDGPPGPKGNMGPQGEPGPPGQQGNPGPQGLPGPQ GPIGPPGEK corresponding to amino acids 1-714 of CA1B_HUMAN (SEQ ID NO:633), which also corresponds to amino acids 1-714 of HUMCA1XIA_P15 (SEQ ID NO:577), and a second amino acid sequence being at least 70%, optionally at least 80%, preferably at least 85%, more preferably at least 90% and most preferably at least 95% homologous to a polypeptide having the sequence MCCNLSFGILIPLQK (SEQ ID NO:1522) corresponding to amino acids 715-729 of HUMCA1XIA_P15 (SEQ ID NO:577), wherein said first amino acid sequence and second amino acid sequence are contiguous and in a sequential order.

2. An isolated polypeptide encoding for a tail of HUMCA1XIA_P15 (SEQ ID NO:577), comprising a polypeptide being at least 70%, optionally at least about 80%, preferably at least about 85%, more preferably at least about 90% and most preferably at least about 95% homologous to the sequence MCCNLSFGILIPLQK (SEQ ID NO:1522) in HUMCA1XIA_P15 (SEQ ID NO:577).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMCA1XIA_P15 (SEQ ID NO:577) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein HUMCA1XIA_P15 (SEQ ID NO:577) sequence provides support for the deduced sequence of this variant protein according to the present invention).

TABLE 10
Amino acid mutations

| SNP position(s) on amino | Alternative | Previously |
|---|---|---|
| acid sequence | amino acid(s) | known SNP? |
| 8 | W -> G | Yes |
| 46 | D -> E | Yes |
| 559 | G -> S | Yes |

The glycosylation sites of variant protein HUMCA1XIA_P15 (SEQ ID NO:577), as compared to the known protein Collagen alpha 1 (SEQ ID NO:633), are described in Table 11 (given according to their position(s) on the amino acid sequence in the first column; the second column indicates whether the glycosylation site is present in the variant protein; and the last column indicates whether the position is different on the variant protein).

TABLE 11
Glycosylation site(s)

| Position(s) on known amino acid | |
|---|---|
| sequence | Present in variant protein? |
| 1640 | no |

Variant protein HUMCA1XIA_P15 (SEQ ID NO:577) is encoded by the following transcript(s): HUMCA1XIA_T17 (SEQ ID NO:46), for which the sequence(s) is/are given at the end of the application. The coding portion of transcript HUMCA1XIA_T17 (SEQ ID NO:46) is shown in bold; this coding portion starts at position 319 and ends at position 2505. The transcript also has the following SNPs as listed in Table 12 (given according to their position on the nucleotide sequence, with the alternative nucleic acid listed; the last column indicates whether the SNP is known or not; the presence of known SNPs in variant protein HUMCA1XIA_P15 (SEQ ID NO:577) sequence provides support for the deduced sequence of this variant protein according to the present invention).

TABLE 12
Nucleic acid SNPs

| SNP position on | Alternative | Previously |
|---|---|---|
| nucleotide sequence | nucleic acid | known SNP? |
| 157 | A -> G | No |
| 241 | T -> A | Yes |
| 340 | T -> G | Yes |
| 456 | T -> G | Yes |
| 1993 | G -> A | Yes |
| 2473 | C -> T | Yes |

Variant protein HUMCA1XIA_P16 (SEQ ID NO:578) according to the present invention has amino acid sequence as given at the end of the application; it is encoded by transcript(s) HUMCA1XIA_T19 (SEQ ID NO:47). An alignment is given to the known protein (Collagen alpha 1 (SEQ ID NO:633)) at the end of the application. One or more alignments to one or more previously published protein sequences are given at the end of the application. A brief description of the relationship of the variant protein according to the present invention to each such aligned protein is as follows:

Comparison Report Between HUMCA1XIA_P16 (SEQ ID NO:578) and CA1B_HUMAN (SEQ ID NO:633):

1. An isolated chimeric polypeptide encoding for HUMCA1XIA_P16 (SEQ ID NO:578), comprising a first amino acid sequence being at least 90% homologous to MEPWSSRWKTKRWLWDFTVTTLALTFLFQAREVRGAAPVDVLKALDFHNSPEGISKTTGFCTNRKNSKG SDTAYRVSKQAQLSAPTKQLFPGGTFPEDFSILFTVKPKKGIQSFLLSIYNEHGIQQIGVEVGRSPVFLFEDH TGKPAPEDYPLFRTVNIADGKWHRVAISVEKKTVTMIVDCKKKTTKPLDRSERAIVDTNGITVFGTRILDE EVFEGDIQQFLITGDPKAAYDYCEHYSPDCDSSAPKAAQAQEPQIDEYAPEDIIEYDYEYGEAEYKEAESVT EGPTVTEETIAQTEANIVDDFQEYNYGTMESYQTEAPRHVSGTNEPNPVEEIFTEEYLTGEDYDSQRKNSE DTLYENKEIDGRDSDLLVDGDLGEYDFYEYKEYEDKPTSPPNEEFGPGVPAETDITETSINGHGAYGEKGQ KGEPAVVEPGMLVEGPPGPAGPAGIMGPPGLQGPTGPPGDPGDRGPPGRPGLPGADGLPGPPGTMLMLPF RYGGDGSKGPTISAQEAQAQAILQQARIALRGPPGPMGLTGRPGPVGGPGSSGAKGESGDPGPQGPRGVQ GPPGPTGKPGKRGRPGADGGRGMPGEPGAKGDRGFDGLPGLPGDKGHRGERGPQGPPGPPGDDGMRGE DGEIGPRGLPGEA corresponding to amino acids 1-648 of CA1B_HUMAN (SEQ ID NO:633), which also corresponds to amino acids 1-648 of HUMCA1XIA_P16 (SEQ ID NO:578), a second amino acid sequence being at least 90% homologous to GMAGVDGPPGPKGNMGPQGEPGPPGQQGNPGPQGLPGPQGPIGPPGEK corresponding to amino acids 667-714 of CA1B HUMAN (SEQ ID NO:633), which also corresponds to amino acids 649-696 of HUMCA1XIA_P16 (SEQ ID NO:578), and a third amino acid sequence being at least 70%, optionally at least 80%, preferably at least 85%, more preferably at least 90% and most preferably at least 95% homologous to a polypeptide having the sequence VSFSFSLFYKKVIKFACDKRFVGRHDERKVVKLSLPLYLIYE (SEQ ID NO:1523) corresponding to amino acids 649-696 of HUMCA1XIA_P16 (SEQ ID NO:578), wherein said first amino acid sequence, second amino acid sequence and third amino acid sequence are contiguous and in a sequential order.

2. An isolated chimeric polypeptide encoding for an edge portion of HUMCA1XIA_P16 (SEQ ID NO:578), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AG, having a structure as follows: a sequence starting from any of amino acid numbers 648−x to 648; and ending at any of amino acid numbers 649+((n−2)−x), in which x varies from 0 to n−2.

3. An isolated polypeptide encoding for a tail of HUMCA1XIA_P16 (SEQ ID NO:578), comprising a polypeptide being at least 70%, optionally at least about 80%, preferably at least about 85%, more preferably at least about 90% and most preferably at least about 95% homologous to the sequence VSFSFSLFYKKVIKFACDKRFVGRHDERKVVKLSLPLYLIYE (SEQ ID NO:1523) in HUMCA1XIA_P16 (SEQ ID NO:578).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMCA1XIA_P16 (SEQ ID NO:578) also has the following non-silent SNPs (Single Nucleotide Polymorphisms) as listed in Table 13, (given according to their position(s) on the amino acid sequence, with the alternative amino acid(s) listed; the last column indicates whether the SNP is known or not; the presence of known SNPs in variant protein HUMCA1XIA_P16 (SEQ ID NO:578) sequence provides support for the deduced sequence of this variant protein according to the present invention).

TABLE 13
Amino acid mutations

| SNP position(s) on amino | Alternative | Previously |
|---|---|---|
| acid sequence | amino acid(s) | known SNP? |
| 8 | W -> G | Yes |
| 46 | D -> E | Yes |
| 559 | G -> S | Yes |

The glycosylation sites of variant protein HUMCA1XIA_P16 (SEQ ID NO:578), as compared to the known protein Collagen alpha 1 (SEQ ID NO:633), are described in Table 14 (given according to their position(s) on the amino acid sequence in the first column; the second column indicates whether the glycosylation site is present in the variant protein; and the last column indicates whether the position is different on the variant protein).

TABLE 14
Glycosylation site(s)

| Position(s) on known | Present in | Position in |
|---|---|---|
| amino acid sequence | variant protein? | variant protein? |
| 1640 | no | |

Variant protein HUMCA1XIA_P16 (SEQ ID NO:578) is encoded by the following transcript(s): HUMCA1XIA_T19 (SEQ ID NO:47), for which the sequence(s) is/are given at the end of the application. The coding portion of transcript HUMCA1XIA_T19 (SEQ ID NO:47) is shown in bold; this coding portion starts at position 319 and ends at position 2532. The transcript also has the following SNPs as listed in Table 15 (given according to their position on the nucleotide sequence, with the alternative nucleic acid listed; the last column indicates whether the SNP is known or not; the presence of known SNPs in variant protein HUMCA1XIA_P16 (SEQ ID NO:578) sequence provides support for the deduced sequence of this variant protein according to the present invention).

TABLE 15
Nucleic acid SNPs

| SNP position on | Alternative | Previously |
|---|---|---|
| nucleotide sequence | nucleic acid | known SNP? |
| 157 | A -> G | No |
| 241 | T -> A | Yes |
| 340 | T -> G | Yes |
| 456 | T -> G | Yes |
| 1993 | G -> A | Yes |
| 2606 | C -> A | Yes |
| 2677 | T -> G | Yes |
| 2849 | C -> T | Yes |

Variant protein HUMCA1XIA_P17 (SEQ ID NO:579) according to the present invention has an amino acid sequence as given at the end of the application; it is encoded by transcript(s) HUMCA1XIA_T20 (SEQ ID NO:48). An alignment is given to the known protein (Collagen alpha 1 (SEQ ID NO:633)) at the end of the application. One or more alignments to one or more previously published protein sequences are given at the end of the application. A brief description of the relationship of the variant protein according to the present invention to each such aligned protein is as follows:

Comparison Report Between HUMCA1XIA_P17 (SEQ ID NO:579) and CA1B_HUMAN (SEQ ID NO:633):

1. An isolated chimeric polypeptide encoding for HUMCA1XIA_P17 (SEQ ID NO:579), comprising a first amino acid sequence being at least 90% homologous to MEPWSSRWKTKRWLWDFTVTTLALTFLFQAREVRGAAPVDVLKALDFHNSPEGISKTTGFCTNRKNSKG SDTAYRVSKQAQLSAPTKQLFPGGTFPEDFSILFTVKPKKGIQSFLLSIYNEHGIQQIGVEVGRSPVFLFEDH TGKPAPEDYPLFRTVNIADGKWHRVAISVEKKTVTMIVDCKKKTTKPLDRSERAIVDTNGITVFGTRILDE EVFEGDIQQFLITGDPKAAYDYCEHYSPDCDSSAPKAAQAQEPQIDE corresponding to amino acids 1-260 of CA1B_HUMAN (SEQ ID NO:633), which also corresponds to amino acids 1-260 of HUMCA1XIA_P17 (SEQ ID NO:579), and a second amino acid sequence being at least 70%, optionally at least 80%, preferably at least 85%, more preferably at least 90% and most preferably at least 95% homologous to a polypeptide having the sequence VRSTRPEKVFVFQ (SEQ ID NO:1524) corresponding to amino acids 261-273 of HUMCA1XIA_P17 (SEQ ID NO:579), wherein said first amino acid sequence and second amino acid sequence are contiguous and in a sequential order.

2. An isolated polypeptide encoding for a tail of HUMCA1XIA_P17 (SEQ ID NO:579), comprising a polypeptide being at least 70%, optionally at least about 80%, preferably at least about 85%, more preferably at least about 90% and most preferably at least about 95% homologous to the sequence VRSTRPEKVFVFQ (SEQ ID NO:1524) in HUMCA1XIA_P17 (SEQ ID NO:579).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMCA1XIA_P17 (SEQ ID NO:579) also has the following non-silent SNPs (Single Nucleotide Polymorphisms) as listed in Table 16, (given according to their position(s) on the amino acid sequence, with the alternative amino acid(s) listed; the last column indicates whether the SNP is known or not; the presence of known SNPs in variant protein HUMCA1XIA_P17 (SEQ ID NO:579) sequence provides support for the deduced sequence of this variant protein according to the present invention).

TABLE 16
Amino acid mutations

| SNP position(s) on | Alternative | Previously |
|---|---|---|
| amino acid sequence | amino acid(s) | known SNP? |
| 8 | W -> G | Yes |
| 46 | D -> E | Yes |

The glycosylation sites of variant protein HUMCA1XIA_P17 (SEQ ID NO:579), as compared to the known protein Collagen alpha 1 (SEQ ID NO:633), are described in Table 17 (given according to their position(s) on the amino acid sequence in the first column; the second column indicates whether the glycosylation site is present in the variant protein; and the last column indicates whether the position is different on the variant protein).

TABLE 17
Glycosylation site(s)

| Position(s) on known | Present in |
|---|---|
| amino acid sequence | variant protein? |
| 1640 | no |

Variant protein HUMCA1XIA_P17 (SEQ ID NO:579) is encoded by the following transcript(s): HUMCA1XIA_T20 (SEQ ID NO:48), for which the sequence(s) is/are given at the end of the application. The coding portion of transcript HUMCA1XIA_T20 (SEQ ID NO:48) is shown in bold; this coding portion starts at position 319 and ends at position 1137. The transcript also has the following SNPs as listed in Table 18 (given according to their position on the nucleotide sequence, with the alternative nucleic acid listed; the last column indicates whether the SNP is known or not; the presence of known SNPs in variant protein HUMCA1XIA_P17 (SEQ ID NO:579) sequence provides support for the deduced sequence of this variant protein according to the present invention).

TABLE 18
Nucleic acid SNPs

| SNP position on | Alternative | Previously |
|---|---|---|
| nucleotide sequence | nucleic acid | known SNP? |
| 157 | A -> G | No |
| 241 | T -> A | Yes |
| 340 | T -> G | Yes |
| 456 | T -> G | Yes |
| 1150 | A -> C | Yes |

As noted above, cluster HUMCA1XIA features 46 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HUMCA1XIA_node_{—}0 (SEQ ID NO:317) according to the present invention is supported by 13 libraries. The number of libraries was determined as previously described. This segment can be found in the following transcript(s): HUMCA1XIA_T16 (SEQ ID NO:45), HUMCA1XIA_T17 (SEQ ID NO:46), HUMCA1XIA_T19 (SEQ ID NO:47) and HUMCA1XIA_T20 (SEQ ID NO:48). Table 19 below describes the starting and ending position of this segment on each transcript.

TABLE 19
Segment location on transcripts

| | Segment | Segment |
|---|---|---|
| | starting | ending |
| Transcript name | position | position |
| HUMCA1XIA_T16 (SEQ ID NO: 45) | 1 | 424 |
| HUMCA1XIA_T17 (SEQ ID NO: 46) | 1 | 424 |
| HUMCA1XIA_T19 (SEQ ID NO: 47) | 1 | 424 |
| HUMCA1XIA_T20 (SEQ ID NO: 48) | 1 | 424 |

Segment cluster HUMCA1XIA_node_{—}2 (SEQ ID NO:318) according to the present invention is supported by 9 libraries. The number of libraries was determined as previously described. This segment can be found in the following transcript(s): HUMCA1XIA_T16 (SEQ ID NO:45), HUMCA1XIA_T17 (SEQ ID NO:46), HUMCA1XIA_T19 (SEQ ID NO:47) and HUMCA1XIA_T20 (SEQ ID NO:48). Table 20 below describes the starting and ending position of this segment on each transcript.

TABLE 20
Segment location on transcripts

| | Segment | Segment |
|---|---|---|
| | starting | ending |
| Transcript name | position | position |
| HUMCA1XIA_T16 (SEQ ID NO: 45) | 425 | 592 |
| HUMCA1XIA_T17 (SEQ ID NO: 46) | 425 | 592 |
| HUMCA1XIA_T19 (SEQ ID NO: 47) | 425 | 592 |
| HUMCA1XIA_T20 (SEQ ID NO: 48) | 425 | 592 |

Segment cluster HUMCA1XIA_node_{—}4 (SEQ ID NO:319) according to the present invention is supported by 5 libraries. The number of libraries was determined as previously described. This segment can be found in the following transcript(s): HUMCA1XIA_T16 (SEQ ID NO:45), HUMCA1XIA_T17 (SEQ ID NO:46), HUMCA1XIA_T19 (SEQ ID NO:47) and HUMCA1XIA_T20 (SEQ ID NO:48). Table 21 below describes the starting and ending position of this segment on each transcript.

TABLE 21
Segment location on transcripts

| | Segment | Segment |
|---|---|---|
| | starting | ending |
| Transcript name | position | position |
| HUMCA1XIA_T16 (SEQ ID NO: 45) | 593 | 806 |
| HUMCA1XIA_T17 (SEQ ID NO: 46) | 593 | 806 |
| HUMCA1XIA_T19 (SEQ ID NO: 47) | 593 | 806 |
| HUMCA1XIA_T20 (SEQ ID NO: 48) | 593 | 806 |

Microarray (chip) data is also available for this segment as follows. As described above with regard to the cluster itself, various oligonucleotides were tested for being differentially expressed in various disease conditions, particularly cancer. The following oligonucleotides were found to hit this segment (in relation to colon cancer), shown in Table 22.

TABLE 22
Oligonucleotides related to this segment

| | Overexpressed | Chip |
|---|---|---|
| Oligonucleotide name | in cancers | reference |
| HUMCA1XIA_0_18_0 | colorectal cancer | Colon |
| (SEQ ID NO: 1412) | | |

Segment cluster HUMCA1XIA_node_{—}6 (SEQ ID NO:320) according to the present invention is supported by 5 libraries. The number of libraries was determined as previously described. This segment can be found in the following transcript(s): HUMCA1XIA_T16 (SEQ ID NO:45), HUMCA1XIA_T17 (SEQ ID NO:46), HUMCA1XIA_T19 (SEQ ID NO:47) and HUMCA1XIA_T20 (SEQ ID NO:48). Table 23 below describes the starting and ending position of this segment on each transcript.

TABLE 23
Segment location on transcripts

| | Segment | Segment |
|---|---|---|
| | starting | ending |
| Transcript name | position | position |
| HUMCA1XIA_T16 (SEQ ID NO: 45) | 807 | 969 |
| HUMCA1XIA_T17 (SEQ ID NO: 46) | 807 | 969 |
| HUMCA1XIA_T19 (SEQ ID NO: 47) | 807 | 969 |
| HUMCA1XIA_T20 (SEQ ID NO: 48) | 807 | 969 |

Microarray (chip) data is also available for this segment as follows. As described above with regard to the cluster itself, various oligonucleotides were tested for being differentially expressed in various disease conditions, particularly cancer. The following oligonucleotides were found to hit this segment, shown in Table 24.

TABLE 24
Oligonucleotides related to this segment

| | | Chip |
|---|---|---|
| Oligonucleotide name | Overexpressed in cancers | reference |
| HUMCA1XIA_0_18_0 | breast malignant tumors | BRS |
| (SEQ ID NO: 1412) | | |
| HUMCA1XIA_0_18_0 | colorectal cancer | Colon |
| (SEQ ID NO: 1412) | | |
| HUMCA1XIA_0_18_0 | lung malignant tumors | LUN |
| (SEQ ID NO: 1412) | | |

Segment cluster HUMCA1XIA_node_{—}8 (SEQ ID NO:321) according to the present invention is supported by 5 libraries. The number of libraries was determined as previously described. This segment can be found in the following transcript(s): HUMCA1XIA_T16 (SEQ ID NO:45), HUMCA1XIA_T17 (SEQ ID NO:46), HUMCA1XIA_T19 (SEQ ID NO:47) and HUMCA1XIA_T20 (SEQ ID NO:48). Table 25 below describes the starting and ending position of this segment on each transcript.

TABLE 25
Segment location on transcripts

| | Segment | Segment |
|---|---|---|
| | starting | ending |
| Transcript name | position | position |
| HUMCA1XIA_T16 (SEQ ID NO: 45) | 970 | 1098 |
| HUMCA1XIA_T17 (SEQ ID NO: 46) | 970 | 1098 |
| HUMCA1XIA_T19 (SEQ ID NO: 47) | 970 | 1098 |
| HUMCA1XIA_T20 (SEQ ID NO: 48) | 970 | 1098 |

Segment cluster HUMCA1XIA_node_{—}9 (SEQ ID NO:322) according to the present invention is supported by 2 libraries. The number of libraries was determined as previously described. This segment can be found in the following transcript(s): HUMCA1XIA_T20 (SEQ ID NO:48). Table 26 below describes the starting and ending position of this segment on each transcript.

TABLE 26
Segment location on transcripts

| | Segment | Segment |
|---|---|---|
| | starting | ending |
| Transcript name | position | position |
| HUMCA1XIA_T20 (SEQ ID NO: 48) | 1099 | 1271 |

Segment cluster HUMCA1XIA_node_{—}18 (SEQ ID NO:323) according to the present invention is supported by 6 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 27 below describes the starting and ending position of this segment on each transcript.

TABLE 27
Segment location on transcripts

| | Segment | Segment |
|---|---|---|
| | starting | ending |
| Transcript name | position | position |
| HUMCA1XIA_T16 (SEQ ID NO: 45) | 1309 | 1522 |
| HUMCA1XIA_T17 (SEQ ID NO: 46) | 1309 | 1522 |
| HUMCA1XIA_T19 (SEQ ID NO: 47) | 1309 | 1522 |

Segment cluster HUMCA1XIA_node_{—}54 (SEQ ID NO:324) according to the present invention is supported by 2 libraries. The number of libraries was determined as previously described. This segment can be found in the following transcript(s): HUMCA1XIA_T19 (SEQ ID NO:47). Table 28 below describes the starting and ending position of this segment on each transcript.

TABLE 28
Segment location on transcripts

| | Segment | Segment |
|---|---|---|
| | starting | ending |
| Transcript name | position | position |
| HUMCA1XIA_T19 (SEQ ID NO: 47) | 2407 | 2836 |

Segment cluster HUMCA1XIA_node_{—}55 (SEQ ID NO:325) according to the present invention is supported by 4 libraries. The number of libraries was determined as previously described. This segment can be found in the following transcript(s): HUMCA1XIA_T17 (SEQ ID NO:46) and HUMCA1XIA_T19 (SEQ ID NO:47). Table 29 below describes the starting and ending position of this segment on each transcript.

TABLE 29
Segment location on transcripts

| | Segment | |
|---|---|---|
| | starting | Segment |
| Transcript name | position | ending position |
| HUMCA1XIA_T17 (SEQ ID NO: 46) | 2461 | 2648 |
| HUMCA1XIA_T19 (SEQ ID NO: 47) | 2837 | 3475 |

Segment cluster HUMCA1XIA_node_{—}92 (SEQ ID NO:326) according to the present invention is supported by 2 libraries. The number of libraries was determined as previously described. This segment can be found in the following transcript(s): HUMCA1XIA_T16 (SEQ ID NO:45). Table 31 below describes the starting and ending position of this segment on each transcript.

TABLE 31
Segment location on transcripts

| | Segment | |
|---|---|---|
| | starting | Segment |
| Transcript name | position | ending position |
| HUMCA1XIA_T16 (SEQ ID NO: 45) | 3487 | 3615 |

According to an optional embodiment of the present invention, short segments related to the above cluster are also provided. These segments are up to about 120 bp in length, and so are included in a separate description.

Segment cluster HUMCA1XIA_node_{—}11 (SEQ ID NO:327) according to the present invention is supported by 3 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 32 below describes the starting and ending position of this segment on each transcript.

TABLE 32
Segment location on transcripts

| | Segment | |
|---|---|---|
| | starting | Segment |
| Transcript name | position | ending position |
| HUMCA1XIA_T16 (SEQ ID NO: 45) | 1099 | 1215 |
| HUMCA1XIA_T17 (SEQ ID NO: 46) | 1099 | 1215 |
| HUMCA1XIA_T19 (SEQ ID NO: 47) | 1099 | 1215 |

Segment cluster HUMCA1XIA_node_{—}15 (SEQ ID NO:328) according to the present invention is supported by 5 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 33 below describes the starting and ending position of this segment on each transcript.

TABLE 33
Segment location on transcripts

| | Segment | |
|---|---|---|
| | starting | Segment |
| Transcript name | position | ending position |
| HUMCA1XIA_T16 (SEQ ID NO: 45) | 1216 | 1308 |
| HUMCA1XIA_T17 (SEQ ID NO: 46) | 1216 | 1308 |
| HUMCA1XIA_T19 (SEQ ID NO: 47) | 1216 | 1308 |

Segment cluster HUMCA1XIA_node_{—}19 (SEQ ID NO:329) according to the present invention is supported by 3 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 34 below describes the starting and ending position of this segment on each transcript.

TABLE 34
Segment location on transcripts

| | Segment | |
|---|---|---|
| | starting | Segment |
| Transcript name | position | ending position |
| HUMCA1XIA_T16 (SEQ ID NO: 45) | 1523 | 1563 |
| HUMCA1XIA_T17 (SEQ ID NO: 46) | 1523 | 1563 |
| HUMCA1XIA_T19 (SEQ ID NO: 47) | 1523 | 1563 |

Segment cluster HUMCA1XIA_node_{—}21 (SEQ ID NO:330) according to the present invention is supported by 2 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 35 below describes the starting and ending position of this segment on each transcript.

TABLE 35
Segment location on transcripts

| | Segment | |
|---|---|---|
| | starting | Segment |
| Transcript name | position | ending position |
| HUMCA1XIA_T16 (SEQ ID NO: 45) | 1564 | 1626 |
| HUMCA1XIA_T17 (SEQ ID NO: 46) | 1564 | 1626 |
| HUMCA1XIA_T19 (SEQ ID NO: 47) | 1564 | 1626 |

Segment cluster HUMCA1XIA_node_{—}23 (SEQ ID NO:331) according to the present invention is supported by 3 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 36 below describes the starting and ending position of this segment on each transcript.

TABLE 36
Segment location on transcripts

| | Segment | |
|---|---|---|
| | starting | Segment |
| Transcript name | position | ending position |
| HUMCA1XIA_T16 (SEQ ID NO: 45) | 1627 | 1668 |
| HUMCA1XIA_T17 (SEQ ID NO: 46) | 1627 | 1668 |
| HUMCA1XIA_T19 (SEQ ID NO: 47) | 1627 | 1668 |

Segment cluster HUMCA1XIA_node_{—}25 (SEQ ID NO:332) according to the present invention is supported by 3 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 37 below describes the starting and ending position of this segment on each transcript.

TABLE 37
Segment location on transcripts

| | Segment | |
|---|---|---|
| | starting | Segment |
| Transcript name | position | ending position |
| HUMCA1XIA_T16 (SEQ ID NO: 45) | 1669 | 1731 |
| HUMCA1XIA_T17 (SEQ ID NO: 46) | 1669 | 1731 |
| HUMCA1XIA_T19 (SEQ ID NO: 47) | 1669 | 1731 |

Segment cluster HUMCA1XIA_node_{—}27 (SEQ ID NO:333) according to the present invention is supported by 2 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 38 below describes the starting and ending position of this segment on each transcript.

TABLE 38
Segment location on transcripts

| | Segment | |
|---|---|---|
| | starting | Segment |
| Transcript name | position | ending position |
| HUMCA1XIA_T16 (SEQ ID NO: 45) | 1732 | 1806 |
| HUMCA1XIA_T17 (SEQ ID NO: 46) | 1732 | 1806 |
| HUMCA1XIA_T19 (SEQ ID NO: 47) | 1732 | 1806 |

Segment cluster HUMCA1XIA_node_{—}29 (SEQ ID NO:334) according to the present invention is supported by 3 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 39 below describes the starting and ending position of this segment on each transcript.

TABLE 39
Segment location on transcripts

| | Segment | |
|---|---|---|
| | starting | Segment |
| Transcript name | position | ending position |
| HUMCA1XIA_T16 (SEQ ID NO: 45) | 1807 | 1890 |
| HUMCA1XIA_T17 (SEQ ID NO: 46) | 1807 | 1890 |
| HUMCA1XIA_T19 (SEQ ID NO: 47) | 1807 | 1890 |

Segment cluster HUMCA1XIA_node_{—}31 (SEQ ID NO:335) according to the present invention is supported by 3 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 40 below describes the starting and ending position of this segment on each transcript.

TABLE 40
Segment location on transcripts

| | Segment | |
|---|---|---|
| | starting | Segment |
| Transcript name | position | ending position |
| HUMCA1XIA_T16 (SEQ ID NO: 45) | 1891 | 1947 |
| HUMCA1XIA_T17 (SEQ ID NO: 46) | 1891 | 1947 |
| HUMCA1XIA_T19 (SEQ ID NO: 47) | 1891 | 1947 |

Segment cluster HUMCA1XIA_node_{—}33 (SEQ ID NO:336) according to the present invention is supported by 3 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 41 below describes the starting and ending position of this segment on each transcript.

TABLE 41
Segment location on transcripts

| | Segment | |
|---|---|---|
| | starting | Segment |
| Transcript name | position | ending position |
| HUMCA1XIA_T16 (SEQ ID NO: 45) | 1948 | 2001 |
| HUMCA1XIA_T17 (SEQ ID NO: 46) | 1948 | 2001 |
| HUMCA1XIA_T19 (SEQ ID NO: 47) | 1948 | 2001 |

Segment cluster HUMCA1XIA_node_{—}35 (SEQ ID NO:337) according to the present invention is supported by 4 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 42 below describes the starting and ending position of this segment on each transcript.

TABLE 42
Segment location on transcripts

| | Segment | |
|---|---|---|
| | starting | Segment |
| Transcript name | position | ending position |
| HUMCA1XIA_T16 (SEQ ID NO: 45) | 2002 | 2055 |
| HUMCA1XIA_T17 (SEQ ID NO: 46) | 2002 | 2055 |
| HUMCA1XIA_T19 (SEQ ID NO: 47) | 2002 | 2055 |

Segment cluster HUMCA1XIA_node_{—}37 (SEQ ID NO:338) according to the present invention is supported by 4 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 43 below describes the starting and ending position of this segment on each transcript.

TABLE 43
Segment location on transcripts

| | Segment | |
|---|---|---|
| | starting | Segment |
| Transcript name | position | ending position |
| HUMCA1XIA_T16 (SEQ ID NO: 45) | 2056 | 2109 |
| HUMCA1XIA_T17 (SEQ ID NO: 46) | 2056 | 2109 |
| HUMCA1XIA_T19 (SEQ ID NO: 47) | 2056 | 2109 |

Segment cluster HUMCA1XIA_node_{—}39 (SEQ ID NO:339) according to the present invention is supported by 5 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 44 below describes the starting and ending position of this segment on each transcript.

TABLE 44
Segment location on transcripts

| | Segment | |
|---|---|---|
| | starting | Segment |
| Transcript name | position | ending position |
| HUMCA1XIA_T16 (SEQ ID NO: 45) | 2110 | 2163 |
| HUMCA1XIA_T17 (SEQ ID NO: 46) | 2110 | 2163 |
| HUMCA1XIA_T19 (SEQ ID NO: 47) | 2110 | 2163 |

Segment cluster HUMCA1XIA_node_{—}41 (SEQ ID NO:340) according to the present invention is supported by 4 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 45 below describes the starting and ending position of this segment on each transcript.

TABLE 45
Segment location on transcripts

| | | |
|---|---|---|
| | Segment | Segment |
| | starting | ending |
| Transcript name | position | position |

| | | |
|---|---|---|
| HUMCA1XIA_T16 (SEQ ID NO: 45) | 2164 | 2217 |
| HUMCA1XIA_T17 (SEQ ID NO: 46) | 2164 | 2217 |
| HUMCA1XIA_T19 (SEQ ID NO: 47) | 2164 | 2217 |

Segment cluster HUMCA1XIA_node_{—}43 (SEQ ID NO:341) according to the present invention is supported by 5 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 46 below describes the starting and ending position of this segment on each transcript.

TABLE 46
Segment location on transcripts

| | Segment | Segment |
|---|---|---|
| | starting | ending |
| Transcript name | position | position |
| HUMCA1XIA_T16 (SEQ ID NO: 45) | 2218 | 2262 |
| HUMCA1XIA_T17 (SEQ ID NO: 46) | 2218 | 2262 |
| HUMCA1XIA_T19 (SEQ ID NO: 47) | 2218 | 2262 |

Segment cluster HUMCA1XIA_node_{—}45 (SEQ ID NO:342) according to the present invention is supported by 4 libraries. The number of libraries was determined as previously described. This segment can be found in the following transcript(s): HUMCA1XIA_T16 (SEQ ID NO:45) and HUMCA1XIA_T17 (SEQ ID NO:46). Table 47 below describes the starting and ending position of this segment on each transcript.

TABLE 47
Segment location on transcripts

| | Segment | Segment |
|---|---|---|
| | starting | ending |
| Transcript name | position | position |
| HUMCA1XIA_T16 (SEQ ID NO: 45) | 2263 | 2316 |
| HUMCA1XIA_T17 (SEQ ID NO: 46) | 2263 | 2316 |

Segment cluster HUMCA1XIA_node_{—}47 (SEQ ID NO:343) according to the present invention is supported by 5 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 48 below describes the starting and ending position of this segment on each transcript.

TABLE 48
Segment location on transcripts

| | Segment | Segment |
|---|---|---|
| | starting | ending |
| Transcript name | position | position |
| HUMCA1XIA_T16 (SEQ ID NO: 45) | 2317 | 2361 |
| HUMCA1XIA_T17 (SEQ ID NO: 46) | 2317 | 2361 |
| HUMCA1XIA_T19 (SEQ ID NO: 47) | 2263 | 2307 |

Segment cluster HUMCA1XIA_node_{—}49 (SEQ ID NO:344) according to the present invention is supported by 5 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 49 below describes the starting and ending position of this segment on each transcript.

TABLE 49
Segment location on transcripts

| | Segment | Segment |
|---|---|---|
| | starting | ending |
| Transcript name | position | position |
| HUMCA1XIA_T16 (SEQ ID NO: 45) | 2362 | 2415 |
| HUMCA1XIA_T17 (SEQ ID NO: 46) | 2362 | 2415 |
| HUMCA1XIA_T19 (SEQ ID NO: 47) | 2308 | 2361 |

Segment cluster HUMCA1XIA_node_{—}51 (SEQ ID NO:345) according to the present invention is supported by 7 libraries. The number of libraries was determined as previously described. This segment can be found in the following transcript(s): HUMCA1XIA_T16 (SEQ ID NO:45), HUMCA1XIA_T17 (SEQ ID NO:46) and HUMCA1XIA_T19 (SEQ ID NO:47). Table 50 below describes the starting and ending position of this segment on each transcript.

TABLE 50
Segment location on transcripts

| | Segment | Segment |
|---|---|---|
| | starting | ending |
| Transcript name | position | position |
| HUMCA1XIA_T16 (SEQ ID NO: 45) | 2416 | 2460 |
| HUMCA1XIA_T17 (SEQ ID NO: 46) | 2416 | 2460 |
| HUMCA1XIA_T19 (SEQ ID NO: 47) | 2362 | 2406 |

Segment cluster HUMCA1XIA_node_{—}57 (SEQ ID NO:346) according to the present invention is supported by 4 libraries. The number of libraries was determined as previously described. This segment can be found in the following transcript(s): HUMCA1XIA_T16 (SEQ ID NO:45). Table 51 below describes the starting and ending position of this segment on each transcript.

TABLE 51
Segment location on transcripts

| | Segment | Segment |
|---|---|---|
| | starting | ending |
| Transcript name | position | position |
| HUMCA1XIA_T16 (SEQ ID NO: 45) | 2461 | 2514 |

Segment cluster HUMCA1XIA_node_{—}59 (SEQ ID NO:347) according to the present invention is supported by 3 libraries. The number of libraries was determined as previously described. This segment can be found in the following transcript(s): HUMCA1XIA_T16 (SEQ ID NO:45). Table 52 below describes the starting and ending position of this segment on each transcript.

TABLE 52
Segment location on transcripts

| | Segment | Segment |
|---|---|---|
| | starting | ending |
| Transcript name | position | position |
| HUMCA1XIA_T16 (SEQ ID NO: 45) | 2515 | 2559 |

Segment cluster HUMCA1XIA_node_{—}62 (SEQ ID NO:348) according to the present invention is supported by 3 libraries. The number of libraries was determined as previously described. This segment can be found in the following transcript(s): HUMCA1XIA_T16 (SEQ ID NO:45). Table 53 below describes the starting and ending position of this segment on each transcript.

TABLE 53
Segment location on transcripts

| | Segment | Segment |
|---|---|---|
| | starting | ending |
| Transcript name | position | position |
| HUMCA1XIA_T16 (SEQ ID NO: 45) | 2560 | 2613 |

Segment cluster HUMCA1XIA_node_{—}64 (SEQ ID NO:349) according to the present invention is supported by 4 libraries. The number of libraries was determined as previously described. This segment can be found in the following transcript(s): HUMCA1XIA_T16 (SEQ ID NO:45). Table 54 below describes the starting and ending position of this segment on each transcript.

TABLE 54
Segment location on transcripts

| | Segment | Segment |
|---|---|---|
| | starting | ending |
| Transcript name | position | position |
| HUMCA1XIA_T16 (SEQ ID NO: 45) | 2614 | 2658 |

Segment cluster HUMCA1XIA_node_{—}66 (SEQ ID NO:350) according to the present invention is supported by 4 libraries. The number of libraries was determined as previously described. This segment can be found in the following transcript(s): HUMCA1XIA_T16 (SEQ ID NO:45). Table 55 below describes the starting and ending position of this segment on each transcript.

TABLE 55
Segment location on transcripts

| | Segment | Segment |
|---|---|---|
| | starting | ending |
| Transcript name | position | position |
| HUMCA1XIA_T16 (SEQ ID NO: 45) | 2659 | 2712 |

Segment cluster HUMCA1XIA_node_{—}68 (SEQ ID NO:351) according to the present invention is supported by 7 libraries. The number of libraries was determined as previously described. This segment can be found in the following transcript(s): HUMCA1XIA_T16 (SEQ ID NO:45). Table 56 below describes the starting and ending position of this segment on each transcript.

TABLE 56
Segment location on transcripts

| | Segment | Segment |
|---|---|---|
| | starting | ending |
| Transcript name | position | position |
| HUMCA1XIA_T16 (SEQ ID NO: 45) | 2713 | 2820 |

Segment cluster HUMCA1XIA_node_{—}70 (SEQ ID NO:352) according to the present invention is supported by 6 libraries. The number of libraries was determined as previously described. This segment can be found in the following transcript(s): HUMCA1XIA_T16 (SEQ ID NO:45). Table 57 below describes the starting and ending position of this segment on each transcript.

TABLE 57
Segment location on transcripts

| | Segment | Segment |
|---|---|---|
| | starting | ending |
| Transcript name | position | position |
| HUMCA1XIA_T16 (SEQ ID NO: 45) | 2821 | 2874 |

Segment cluster HUMCA1XIA_node_{—}72 (SEQ ID NO:353) according to the present invention is supported by 6 libraries. The number of libraries was determined as previously described. This segment can be found in the following transcript(s): HUMCA1XIA_T16 (SEQ ID NO:45). Table 58 below describes the starting and ending position of this segment on each transcript.

TABLE 58
Segment location on transcripts

| | Segment | Segment |
|---|---|---|
| | starting | ending |
| Transcript name | position | position |
| HUMCA1XIA_T16 (SEQ ID NO: 45) | 2875 | 2928 |

Segment cluster HUMCA1XIA_node_{—}74 (SEQ ID NO:354) according to the present invention is supported by 5 libraries. The number of libraries was determined as previously described. This segment can be found in the following transcript(s): HUMCA1XIA_T16 (SEQ ID NO:45). Table 59 below describes the starting and ending position of this segment on each transcript.

TABLE 59
Segment location on transcripts

| | Segment | Segment |
|---|---|---|
| | starting | ending |
| Transcript name | position | position |
| HUMCA1XIA_T16 (SEQ ID NO: 45) | 2929 | 2973 |

Segment cluster HUMCA1XIA_node_{—}76 (SEQ ID NO:355) according to the present invention is supported by 6 libraries. The number of libraries was determined as previously described. This segment can be found in the following transcript(s): HUMCA1XIA_T16 (SEQ ID NO:45). Table 60 below describes the starting and ending position of this segment on each transcript.

TABLE 60
Segment location on transcripts

| | Segment | |
|---|---|---|
| | starting | Segment |
| Transcript name | position | ending position |
| HUMCA1XIA_T16 (SEQ ID NO: 45) | 2974 | 3027 |

Segment cluster HUMCA1XIA_node_{—}78 (SEQ ID NO:356) according to the present invention is supported by 6 libraries. The number of libraries was determined as previously described. This segment can be found in the following transcript(s): HUMCA1XIA_T16 (SEQ ID NO:45). Table 61 below describes the starting and ending position of this segment on each transcript.

TABLE 61
Segment location on transcripts

| | Segment | |
|---|---|---|
| | starting | Segment |
| Transcript name | position | ending position |
| HUMCA1XIA_T16 (SEQ ID NO: 45) | 3028 | 3072 |

Segment cluster HUMCA1XIA_node_{—}81 (SEQ ID NO:357) according to the present invention is supported by 8 libraries. The number of libraries was determined as previously described. This segment can be found in the following transcript(s): HUMCA1XIA_T16 (SEQ ID NO:45). Table 62 below describes the starting and ending position of this segment on each transcript.

TABLE 62
Segment location on transcripts

| | Segment | |
|---|---|---|
| | starting | Segment |
| Transcript name | position | ending position |
| HUMCA1XIA_T16 (SEQ ID NO: 45) | 3073 | 3126 |

Segment cluster HUMCA1XIA_node_{—}83 (SEQ ID NO:358) according to the present invention is supported by 7 libraries. The number of libraries was determined as previously described. This segment can be found in the following transcript(s): HUMCA1XIA_T16 (SEQ ID NO:45). Table 63 below describes the starting and ending position of this segment on each transcript.

TABLE 63
Segment location on transcripts

| | Segment | |
|---|---|---|
| | starting | Segment |
| Transcript name | position | ending position |
| HUMCA1XIA_T16 (SEQ ID NO: 45) | 3127 | 3180 |

Segment cluster HUMCA1XIA_node_{—}85 (SEQ ID NO:359) according to the present invention is supported by 6 libraries. The number of libraries was determined as previously described. This segment can be found in the following transcript(s): HUMCA1XIA_T16 (SEQ ID NO:45). Table 64 below describes the starting and ending position of this segment on each transcript.

TABLE 64
Segment location on transcripts

| | Segment | |
|---|---|---|
| | starting | Segment |
| Transcript name | position | ending position |
| HUMCA1XIA_T16 (SEQ ID NO: 45) | 3181 | 3234 |

Segment cluster HUMCA1XIA_node_{—}87 (SEQ ID NO:360) according to the present invention is supported by 10 libraries. The number of libraries was determined as previously described. This segment can be found in the following transcript(s): HUMCA1XIA_T16 (SEQ ID NO:45). Table 65 below describes the starting and ending position of this segment on each transcript.

TABLE 65
Segment location on transcripts

| | Segment | |
|---|---|---|
| | starting | Segment |
| Transcript name | position | ending position |
| HUMCA1XIA_T16 (SEQ ID NO: 45) | 3235 | 3342 |

Segment cluster HUMCA1XIA_node_{—}89 (SEQ ID NO:361) according to the present invention is supported by 9 libraries. The number of libraries was determined as previously described. This segment can be found in the following transcript(s): HUMCA1XIA_T16 (SEQ ID NO:45). Table 66 below describes the starting and ending position of this segment on each transcript.

TABLE 66
Segment location on transcripts

| | Segment | |
|---|---|---|
| | starting | Segment |
| Transcript name | position | ending position |
| HUMCA1XIA_T16 (SEQ ID NO: 45) | 3343 | 3432 |

Segment cluster HUMCA1XIA_node_{—}91 (SEQ ID NO:362) according to the present invention is supported by 11 libraries. The number of libraries was determined as previously described. This segment can be found in the following transcript(s): HUMCA1XIA_T16 (SEQ ID NO:45). Table 67 below describes the starting and ending position of this segment on each transcript.

TABLE 67
Segment location on transcripts

| | Segment | |
|---|---|---|
| | starting | Segment |
| Transcript name | position | ending position |
| HUMCA1XIA_T16 (SEQ ID NO: 45) | 3433 | 3486 |

### Variant Protein Alignment to the Previously Known Protein:

Sequence name: CA1B_HUMAN_V5 (SEQ ID NO: 634)
Sequence documentation:
Alignment of: HUMCA1XIA_P14 (SEQ ID NO: 576) × CA1B_HUMAN_V5 (SEQ ID
NO: 634)
Alignment segment 1/1:

| | | | |
|---|---|---|---|
| Quality: | 10456.00 | Escore: | 0 |
| Matching length: | 1058 | Total length: | 1058 |
| Matching Percent Similarity: | 99.91 | Matching Percent Identity: | 99.91 |
| Total Percent Similarity: | 99.91 | Total Percent Identity: | 99.91 |
| Gaps: | 0 | | |

Alignment:

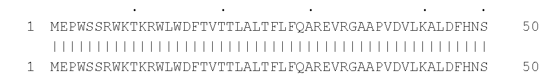

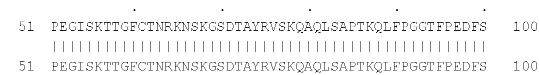

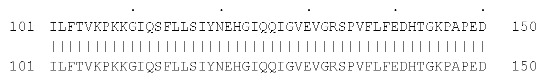

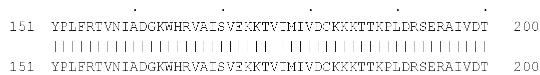

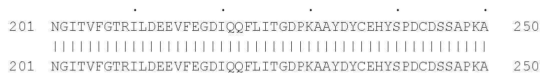

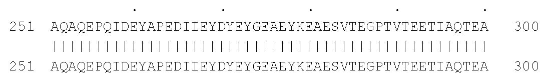

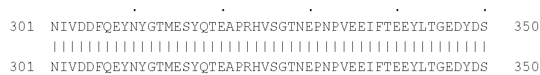

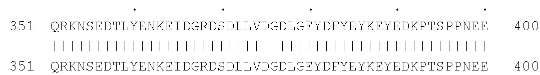

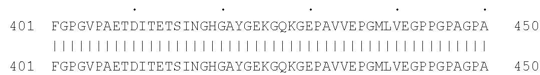

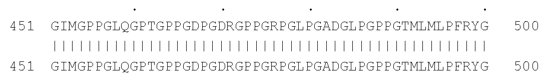

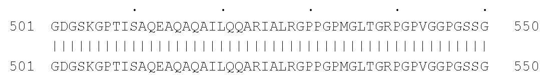

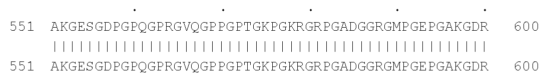

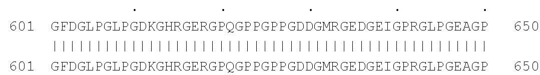

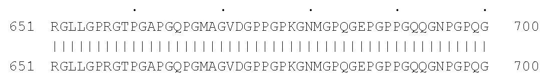

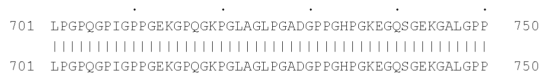

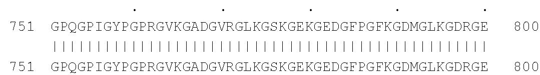

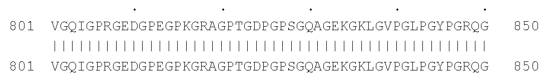

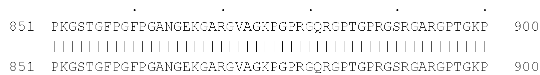

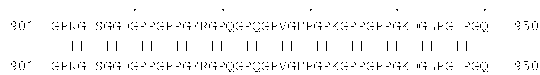

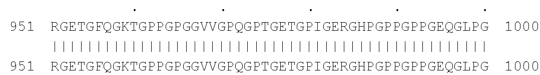

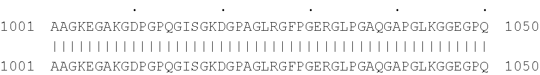

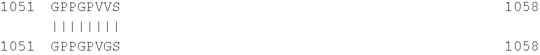

Sequence name: CA1B_HUMAN (SEQ ID NO: 633)
Sequence documentation:
Alignment of:
HUMCA1XIA_P15 (SEQ ID NO: 577) × CA1B_HUMAN (SEQ ID NO: 633)
Alignment segment 1/1:

| | | | |
|---|---|---|---|
| Quality: | 7073.00 | Escore: | 0 |
| Matching length: | 714 | Total length: | 714 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

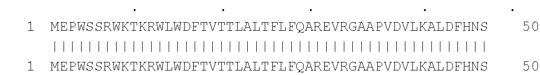

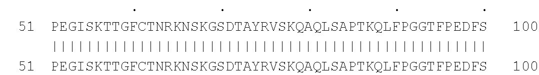

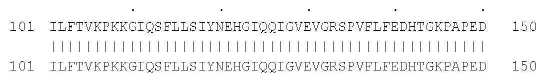

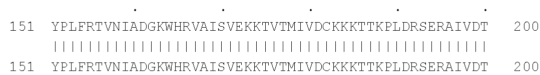

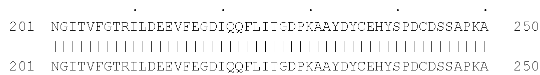

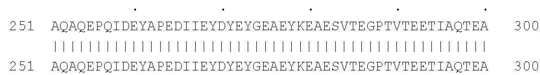

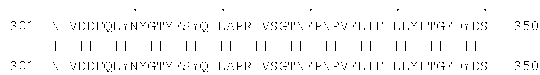

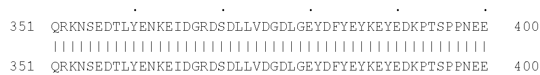

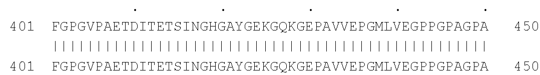

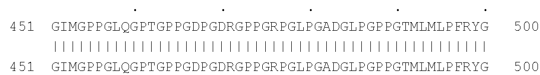

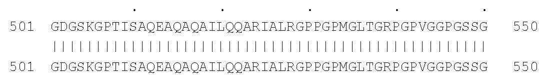

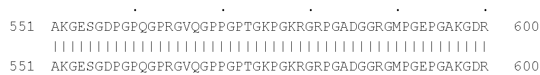

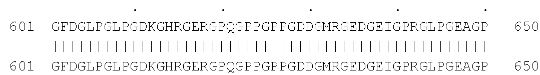

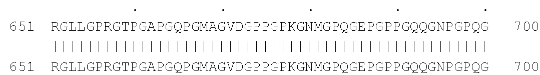

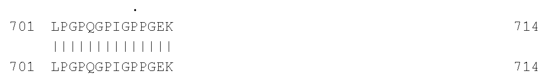

Sequence name: CA1B_HUMAN (SEQ ID NO: 633)
Sequence documentation:
Alignment of:
HUMCA1XIA_P16 (SEQ ID NO: 578) × CA1B_HUMAN (SEQ ID NO: 633)
Alignment segment 1/1:

| | | | |
|---|---|---|---|
| Quality: | 6795.00 | Escore: | 0 |
| Matching length: | 696 | Total length: | 714 |
| Matching Percent Similarity: | 100.00 | Matching Percent Identity: | 100.00 |
| Total Percent Similarity: | 97.48 | Total Percent Identity: | 97.48 |
| Gaps: | 1 | | |

Alignment:

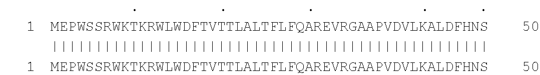

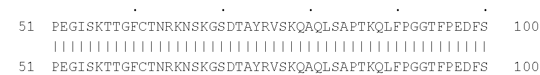

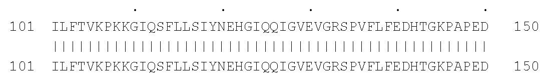

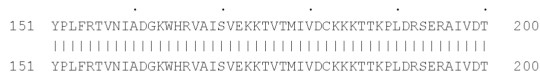

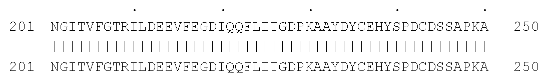

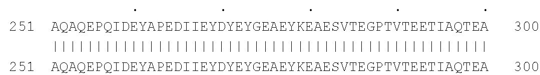

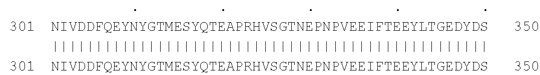

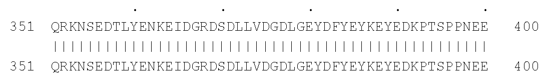

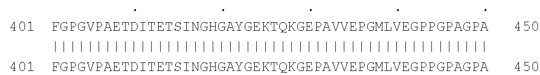

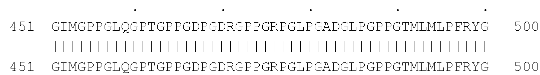

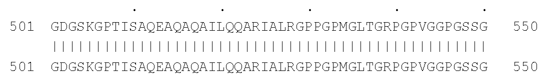

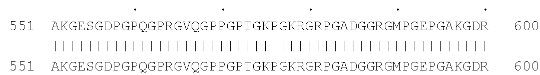

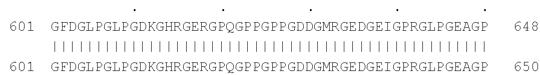

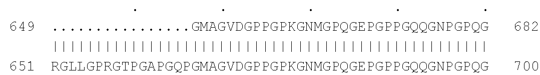

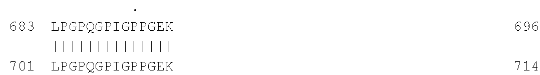

Sequence name: CA1B HUMAN (SEQ ID NO: 633)
Sequence documentation:
Alignment of:
HUMCA1XIA_P17 (SEQ ID NO: 579) × CA1B_HUMAN (SEQ ID NO: 633)
Alignment segment 1/1:

| | | | |
|---|---|---|---|
| Quality: | 2561.00 | Escore: | 0 |
| Matching length: | 260 | Total length: | 260 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

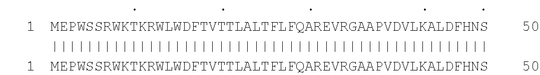

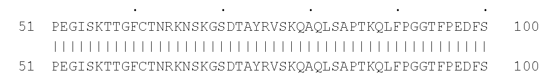

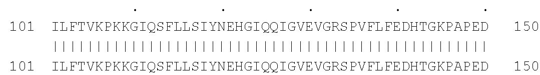

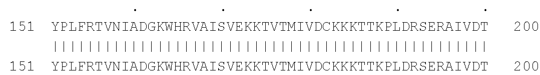

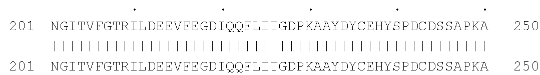

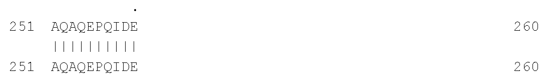

### Experimental Results for Seg55 Amplicon Expression in Cancerous Colon Tissue

Expression of Homo sapiens collagen, type XI, alpha 1 (COL11A1) transcripts detectable by or according to seg55—HUMCA1XIA_seg55 amplicon (SEQ ID NO:1586) and primers HUMCA1XIA_seg55F (SEQ ID NO: 1584) and HUMCA1XIA_seg55R (SEQ ID NO:1585) was measured by real time PCR. Non-detected sample no. 63 was assigned Ct value of 41 and was calculated accordingly. In parallel the expression of several housekeeping genes —HPRT1 (GenBank Accession No. NM_{—}000194 (SEQ ID NO: 1577); amplicon—HPRT1-amplicon (SEQ ID NO: 612)), PBGD (GenBank Accession No. BC019323 (SEQ ID NO: 1576); amplicon—PBGD-amplicon (SEQ ID NO: 531)), and G6PD (GenBank Accession No. NM_{—}000402 (SEQ ID NO: 1578); G6PD amplicon (SEQ ID NO: 615)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 42, 43, 44, 45, 46, 47, 48, 49, 50, 51, 52, 53, 54 and 55, Table 1_{—}1 above), to obtain a value of fold up-regulation for each sample relative to median of the normal samples.

FIG. 77 is a histogram showing over expression of the above-indicated Homo sapiens collagen, type XI, alpha 1 (COL11A1) transcripts in cancerous Colon samples relative to the normal samples.

As is evident from FIG. 77, the expression of Homo sapiens collagen, type XI, alpha 1 (COL11A1) transcripts detectable by the above amplicon in cancer samples was significantly higher than in the non-cancerous samples (sample numbers 42, 43, 44, 45, 46, 47, 48, 49, 50, 51, 52, 53, 54 and 55, Table 1_{—}1 above). Notably an over-expression of at least 5 fold was found in 18 out of 37 adenocarcinoma samples.

Statistical analysis was applied to verify the significance of these results, as described below.

The P value for the difference in the expression levels of Homo sapiens collagen, type XI, alpha 1 (COL11A1) transcripts detectable by the above amplicon in Colon cancer samples versus the normal tissue samples was determined by T test as 3.33e-005.

Threshold of 5 fold over expression was found to differentiate between cancer and normal samples with P value of 2.58e-006 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: HUMCA1XIA_seg55F (SEQ ID NO: 1584) forward primer; and HUMCA1XIA_seg55R (SEQ ID NO: 1585) reverse primer.

The present invention also preferably encompasses any amplicon obtained through the use of any suitable primer pair; for example, for the above experiment, the following amplicon was obtained as a non-limiting illustrative example only of a suitable amplicon: HUMCA1XIA_seg55 (SEQ ID NO: 1586).

Forward Primer
(HUMCA1XIA_seg55F (SEQ ID NO: 1584)):
>HUMCA1XIA_seg55F

(SEQ ID NO: 1584)

TTCTCATAGTATTCCATTGATTGGGTA
Reverse Primer
(HUMCA1XIA_seg55R (SEQ ID NO: 1585)):
>HUMCA1XIA_seg55R

(SEQ ID NO: 1585)

CACCGGTATGGAGAATAGCGA
Amplicon (HUMCA1XIA_seg55) (SEQ ID NO: 1586):
>HUMCA1XIA_seg55

(SEQ ID NO: 1586)

TTCTCATAGTATTCCATTGATTGGGTATACCAGGTTCTGTTTACTTTTAC
TTGGCAGTTGATAGAATAGGTGTAGTTTATACTTTTTCGCTATTCTCCAT
ACCGGTG

### Experimental Results for Seg55 Amplicon Expression in Normal Tissue

Expression of Homo sapiens collagen, type XI, alpha 1 (COL11 A1) transcripts detectable by or according to seg55—HUMCA1XIA_seg55 amplicon (SEQ ID NO: 1586) and primers HUMCA1XIA_seg55F (SEQ ID NO: 1584) and HUMCA1XIA_seg55R (SEQ ID NO: 1585) was measured by real time PCR. In parallel the expression of several housekeeping genes—SDHA (GenBank Accession No. NM_{—}004168 (SEQ ID NO: 1583); amplicon —SDHA-amplicon (SEQ ID NO: 1273)), Ubiquitin (GenBank Accession No. BC000449 (SEQ ID NO: 1582); amplicon—Ubiquitin-amplicon (SEQ ID NO: 1270)) and TATA box (GenBank Accession No. NM_{—}003194 (SEQ ID NO: 1581); TATA amplicon (SEQ ID NO: 1267)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the colon samples (sample numbers 3, 4 and 5, Table 2_{—}1 above), to obtain a value of relative expression of each sample relative to median of the colon samples.

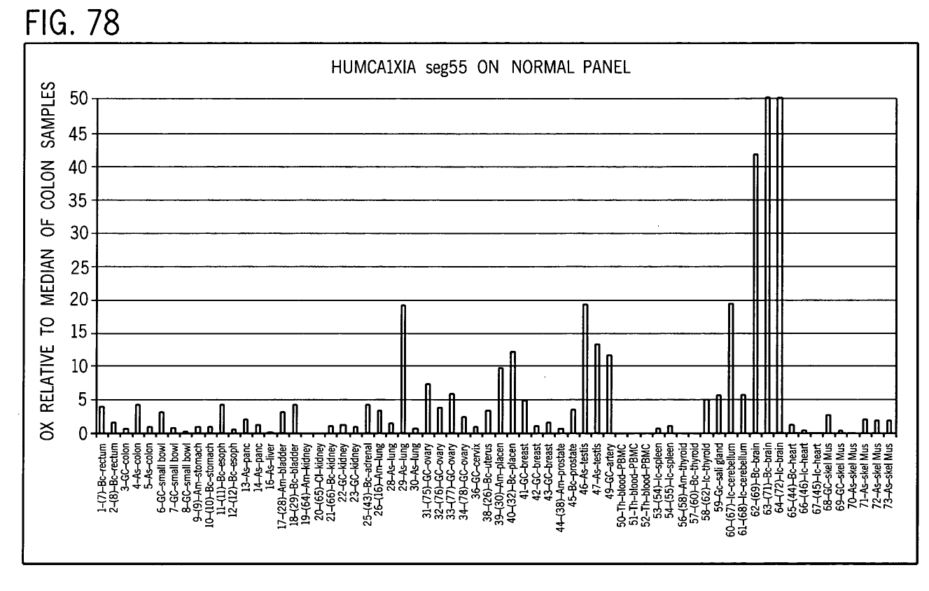

Results are shown in FIG. 78. Low expression was seen in all normal tissues, although a certain level of expression was seen in brain tissue samples.

Forward Primer
(HUMCA1XIA_seg55F (SEQ ID NO: 1584)):
>HUMCA1XIA_seg55F

(SEQ ID NO: 1584)

TTCTCATAGTATTCCATTGATTGGGTA
Reverse Primer
(HUMCA1XIA_seg55R (SEQ ID NO: 1585)):
>HUMCA1XIA_seg55R

(SEQ ID NO: 1585)

CACCGGTATGGAGAATAGCGA
Amplicon (HUMCA1XIA_seg55) (SEQ ID NO: 1586):
>HUMCA1XIA_seg55

(SEQ ID NO: 1586)

TTCTCATAGTATTCCATTGATTGGGTATACCAGGTTCTGTTTACTTTTAC
TTGGCAGTTGATAGAATAGGTGTAGTTTATACTTTTTCGCTATTCTCCAT
ACCGGTG

Expression of Homo sapiens Collagen, Type XI, Alpha 1 (COL11A1) HUMCA1XIA Transcripts which are Detectable by Amplicon as Depicted in Sequence Name HUMCA1XIA_Seg54-55F2R2 (SEQ ID NO: 1589) in Normal and Cancerous Colon Tissues

Expression of Homo sapiens collagen, type XI, alpha 1 (COL11A1) transcripts detectable by or according to seg54-55F2R2—HUMCA1XIA_seg54-55F2R2 (SEQ ID NO: 1589) amplicon and primers HUMCA1XIA_seg54-55F2 (SEQ ID NO: 1587) and HUMCA1XIA_seg54-55R2 (SEQ ID NO: 1588) was measured by real time PCR. Non-detected samples (sample(s) no. 28, 36 and 63) were assigned Ct value of 41 and were calculated accordingly. In parallel the expression of several housekeeping genes —HPRT1 (GenBank Accession No. NM_{—}000194 (SEQ ID NO: 1577); amplicon—HPRT1-amplicon (SEQ ID NO: 612)), PBGD (GenBank Accession No. BC019323 (SEQ ID NO: 1576); amplicon—PBGD-amplicon (SEQ ID NO: 531)), RPS27A (GenBank Accession No. NM_{—}002954 (SEQ ID NO: 1579); RPS27A amplicon (SEQ ID NO: 1261)) and G6PD (GenBank Accession No. NM_{—}000402 (SEQ ID NO: 1578); G6PD amplicon (SEQ ID NO: 615)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 42, 43, 44, 45, 46, 47, 48, 49, 50, 51, 52, 53, 54 and 55, Table 1_{—}1 above), to obtain a value of fold up-regulation for each sample relative to median of the normal samples.

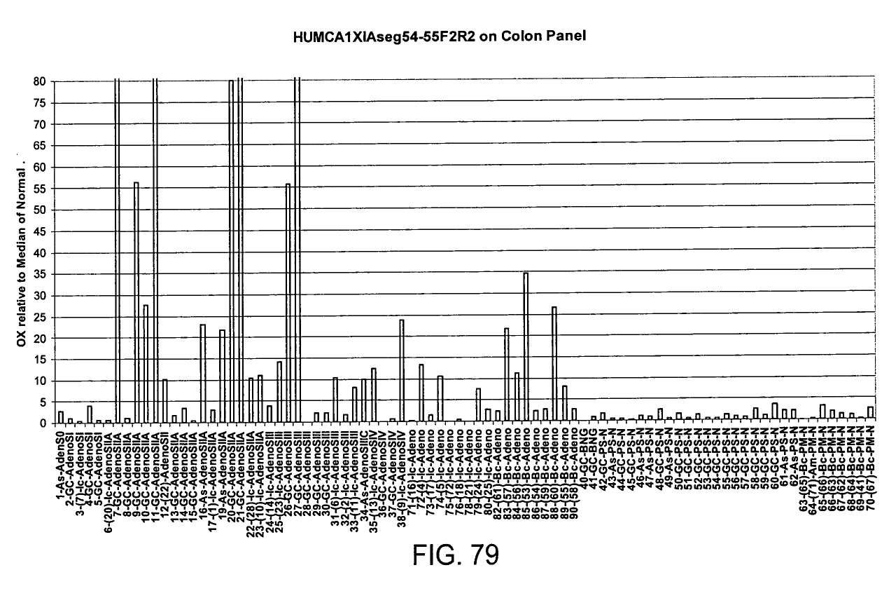

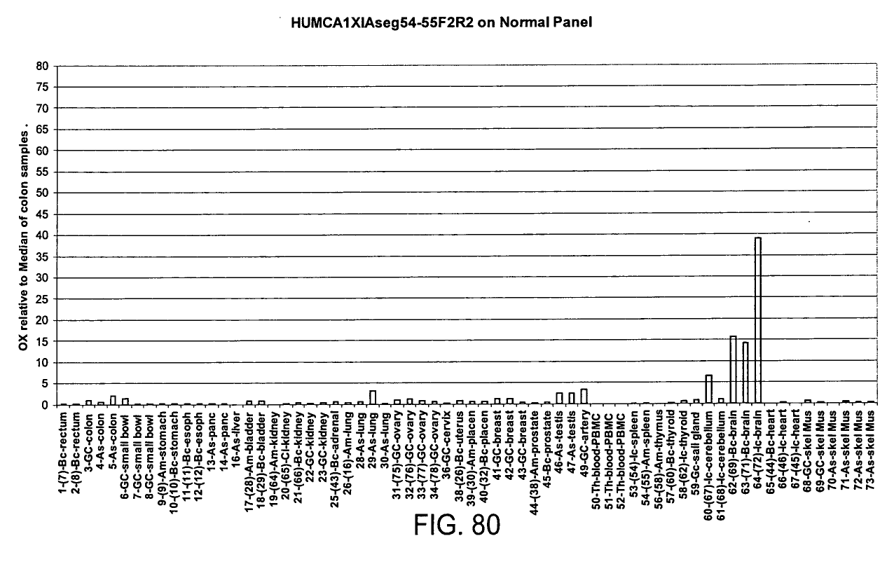

FIG. 79 is a histogram showing over expression of the above-indicated Homo sapiens collagen, type XI, alpha 1 (COL11A1) transcripts in cancerous Colon samples relative to the normal samples.

As is evident from FIG. 79, the expression of Homo sapiens collagen, type XI, alpha 1 (COL11A1) transcripts detectable by the above amplicon in cancer samples was significantly higher than in the non-cancerous samples (sample numbers 42, 43, 44, 45, 46, 47, 48, 49, 50, 51, 52, 53, 54 and 55, Table 1_{—}1 above). Notably an over-expression of at least 5 fold was found in 27 out of 55 adenocarcinoma samples.

Statistical analysis was applied to verify the significance of these results, as described below.

The P value for the difference in the expression levels of Homo sapiens collagen, type XI, alpha 1 (COL11A1) transcripts detectable by the above amplicon in Colon cancer samples versus the normal tissue samples was determined by T test as 7.01e-005.

Threshold of 5 fold over expression was found to differentiate between cancer and normal samples with P value of 5.09e-007 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: HUMCA1XIA_seg54-55F2 (SEQ ID NO: 1587) forward primer; and HUMCA1XIA_seg54-55R2 (SEQ ID NO: 1588) reverse primer.

The present invention also preferably encompasses any amplicon obtained through the use of any suitable primer pair; for example, for the above experiment, the following amplicon was obtained as a non-limiting illustrative example only of a suitable amplicon: HUMCA1XIA_seg54-55F2R2 (SEQ ID NO: 1588).

Forward Primer
(HUMCA1XIA_seg54-55F2 (SEQ ID NO: 1587)):
>HUMCA1XIA_seg54-55F2

(SEQ ID MO: 1587)

TCGAAGTGTAATTTAAATACTATAAATATTCCCTT
Reverse Primer
(HUMCA1XIA_seg54-55R2 (SEQ ID NO: 1588)):
>HUMCA1XIA_seg54-55R2

(SEQ ID NO: 1588)

GGAAACATGGACTGAATATTAACACG
Amplicon
(HUMCA1XIA_seg54-55F2R2 (SEQ ID NO: 1589)):
>HUMCA1XIA_seg54-55F2R2

(SEQ ID NO: 1589)

TCGAAGTGTAATTTAAATACTATAAATATTCCCTTGTTTTAAGTGTACAA
GTGAATTATTTTTACTAAATTTACAGATGTGCTGCAATCTAAGTTTCGGA
ATACTTATACCACTCCAGAAATAATCCTCGTGTTAATATTCAGTCCATGT
TTCC

Expression of Homo sapiens Collagen, Type XI, Alpha 1 (COL11A1) HUMCA1XIA Transcripts which are Detectable by Amplicon as Depicted in Sequence Name HUMCA1XIA_Seg54-55F2R2 (SEQ ID NO:1589) in Different Normal Tissues

Expression of Homo sapiens collagen, type XI, alpha 1 (COL11A1) transcripts detectable by or according to seg54-55F2R2—HUMCA1XIA_seg54-55F2R2 amplicon (SEQ ID NO: 1589) and primers HUMCA1XIA_seg54-55F2 (SEQ ID NO: 1587) and HUMCA1XIA_seg54-55R2 (SEQ ID NO: 1588) was measured by real time PCR. Non-detected samples (samples no. 16, 19, 50, 51, 52, 56, 65, 67 and 70) were assigned Ct value of 41 and were calculated accordingly. In parallel the expression of several housekeeping genes—SDHA (GenBank Accession No. NM_{—}004168 (SEQ ID NO: 1583); amplicon—SDHA-amplicon (SEQ ID NO: 1273)), Ubiquitin (GenBank Accession No. BC000449 (SEQ ID NO: 1582); amplicon—Ubiquitin-amplicon (SEQ ID NO: 1270)), RPL19 (GenBank Accession No. NM_{—}000981 (SEQ ID NO: 1580); RPL19 amplicon (SEQ ID NO: 1264)) and TATA box (GenBank Accession No. NM_{—}003194 (SEQ ID NO: 1581); TATA amplicon (SEQ ID NO: 1267)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the colon samples (sample numbers 3, 4 and 5, Table 2_{—}1 above), to obtain a value of relative expression of each sample relative to median of the colon samples.

Forward Primer
(HUMCA1XIA_seg54-55F2 (SEQ ID NO: 1587)):
>HUMCA1XIA_seg54-55F2

(SEQ ID NO: 1587)

TCGAAGTGTAATTTAAATACTATAAATATTCCCTT
Reverse Primer
(HUMCA1XIA_seg54-55R2 (SEQ ID NO: 1588)):
>HUMCA1XIA_seg54-55R2

(SEQ ID NO: 1588)

GGAAACATGGACTGAATATTAACACG
Amplicon
(HUMCA1XIA_seg54-55F2R2) (SEQ ID NO: 1589):
>HUMCA1XIA_seg54-55F2R2

(SEQ ID NO: 1589)

TCGAAGTGTAATTTAAATACTATAAATATTCCCTTGTTTTAAGTGTACAA
GTGAATTATTTTTACTAAATTTACAGATGTGCTGCAATCTAAGTTTCGGA
ATACTTATACCACTCCAGAAATAATCCTCGTGTTAATATTCAGTCCATGT
TTCC

The results are shown in FIG. 80, demonstrating expression of Homo sapiens collagen, type XI, alpha 1 (COL11A1) HUMCA1XIA transcripts which are detectable by amplicon as depicted in sequence name HUMCA1XIA_seg54-55F2R2 (SEQ ID NO: 1589) in different normal tissues.

Expression of Homo sapiens Collagen, Type XI, Alpha 1 (COL11A1) HUMCA1XIA Transcripts which are Detectable by Amplicon as Depicted in Sequence Name HUMCA1XIA_Seg52-56F1R1 (SEQ ID NO: 1592) in Normal and Cancerous Colon Tissues

Expression of Homo sapiens collagen, type XI, alpha 1 (COL11A1) transcripts detectable by or according to seg52-56F1R1—HUMCA1XIA_seg52-56F1R1 (SEQ ID NO: 1592) amplicon and primers HUMCA1XIA_seg52-56F1 (SEQ ID NO: 1590) and HUMCA1XIA_seg52-56R1 (SEQ ID NO: 1591) was measured by real time PCR. In parallel the expression of several housekeeping genes —HPRT1 (GenBank Accession No. NM_{—}000194 (SEQ ID NO: 1577); amplicon—HPRT1-amplicon (SEQ ID NO: 612)), PBGD (GenBank Accession No. BC019323 (SEQ ID NO: 1576); amplicon—PBGD-amplicon (SEQ ID NO: 531)), RPS27A (GenBank Accession No. NM_{—}002954 (SEQ ID NO: 1579); RPS27A amplicon (SEQ ID NO: 1261)) and G6PD (GenBank Accession No. NM_{—}000402 (SEQ ID NO: 1578); G6PD amplicon (SEQ ID NO: 615)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the normal samples (sample numbers 42-70, Table 1_{—}1 above), to obtain a value of fold differential expression for each sample relative to median of the normal samples.

In one experiment that was carried out no differential expression in the cancerous samples relative to the normal samples was observed.

Primer pairs are also optionally and preferably encompassed within the present invention; for example, for the above experiment, the following primer pair was used as a non-limiting illustrative example only of a suitable primer pair: HUMCA1XIA_seg52-56F1 (SEQ ID NO: 1590) forward primer; and HUMCA1XIA_seg52-56R1 (SEQ ID NO: 1591) reverse primer.

The present invention also preferably encompasses any amplicon obtained through the use of any suitable primer pair; for example, for the above experiment, the following amplicon was obtained as a non-limiting illustrative example only of a suitable amplicon: HUMCA1XIA_seg52-56F1R1 (SEQ ID NO: 1592).

Forward Primer
(HUMCA1XIA_seg52-56F1 (SEQ ID NO: 1590)):
>HUMCA1XIA_seg52-56F1

(SEQ ID NO: 1590)

GGTCTTCCTGGTCCACAAGGT
Reverse Primer
(HUMCA1XIA_seg52-56R1 (SEQ ID NO: 1591)):
>HUMCA1XIA_seg52-56R1

(SEQ ID NO: 1591)

GAATATTAACACGAGGATTATTTCTGGAG
Amplicon
(HUMCA1XIA_seg52-56F1R1 (SEQ ID NO: 1592)):
>HUMCA1XIA_seg52-5GF1R1

(SEQ ID NO: 1592)

GGTCTTCCTGGTCCACAAGGTCCAATTGGTCCTCCTGGTGAAAAAATGTG
CTGCAATCTAAGTTTCGGAATACTTATACCACTCCAGAAATAATCCTCGT
GTTAATATTC

### Description for Cluster HSS100PCB

Cluster HSS100PCB features 1 transcript(s) and 3 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| HSS100PCB_T1 | 49 |

TABLE 2
Segments of interest

| Segment Name | SEQ ID NO: |
|---|---|
| HSS100PCB_node_3 | 363 |
| HSS100PCB_node_4 | 364 |
| HSS100PCB_node_5 | 365 |

TABLE 3
Proteins of interest

| Protein Name | SEQ ID NO: | Corresponding Transcript(s) |
|---|---|---|
| HSS100PCB_P3 | 580 | HSS100PCB_T1 (SEQ ID NO: 49) |

These sequences are variants of the known protein S-100P protein (SwissProt accession identifier S10P_HUMAN), SEQ ID NO: 635, referred to herein as the previously known protein.

The sequence for protein S-100P protein (SEQ ID NO:635) is given at the end of the application, as “S-100P protein amino acid sequence”. Known polymorphisms for this sequence are as shown in Table 4.

TABLE 4
Amino acid mutations for Known Protein

| SNP position(s) on amino | |
|---|---|
| acid sequence | Comment |
| 32 | E -> T |
| 44 | F -> E |

The following GO Annotation(s) apply to the previously known protein. The following annotation(s) were found: calcium binding; protein binding, which are annotation(s) related to Molecular Function.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster HSS100PCB can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 25 and Table 5. This cluster is overexpressed (at least at a minimum level) in the following pathological conditions: a mixture of malignant tumors from different tissues.

TABLE 5
Normal tissue distribution

| Name of Tissue | Number |
|---|---|
| bladder | 41 |
| colon | 37 |
| epithelial | 38 |
| general | 22 |
| kidney | 0 |
| liver | 0 |
| Lung | 18 |
| breast | 0 |
| bone marrow | 0 |
| ovary | 0 |
| pancreas | 0 |
| prostate | 46 |
| stomach | 553 |
| uterus | 13 |

TABLE 6
P values and ratios for expression in cancerous tissue

| Name of Tissue | P1 | P2 | SP1 | R3 | SP2 | R4 |
|---|---|---|---|---|---|---|
| bladder | 3.3e−01 | 2.9e−01 | 2.9e−02 | 2.8 | 3.5e−02 | 2.8 |
| colon | 3.0e−01 | 1.9e−01 | 5.2e−01 | 1.2 | 2.4e−01 | 1.7 |
| epithelial | 4.7e−02 | 1.6e−02 | 2.0e−01 | 1.2 | 6.1e−02 | 1.3 |
| general | 1.1e−03 | 6.8e−05 | 1.4e−02 | 1.5 | 4.9e−04 | 1.7 |
| kidney | 6.5e−01 | 7.2e−01 | 5.8e−01 | 1.7 | 7.0e−01 | 1.4 |
| liver | 9.1e−01 | 4.9e−01 | 1 | 1.0 | 7.7e−02 | 2.1 |
| lung | 6.8e−01 | 7.3e−01 | 2.2e−02 | 2.9 | 1.3e−01 | 1.7 |
| breast | 2.8e−01 | 3.2e−01 | 4.7e−01 | 2.0 | 6.8e−01 | 1.5 |
| bone marrow | 1 | 6.7e−01 | 1 | 1.0 | 2.8e−01 | 2.8 |
| ovary | 2.6e−01 | 3.0e−01 | 4.7e−01 | 2.0 | 5.9e−01 | 1.7 |
| pancreas | 3.3e−01 | 4.4e−01 | 7.6e−02 | 3.7 | 1.5e−01 | 2.8 |
| prostate | 9.1e−01 | 9.3e−01 | 5.8e−01 | 0.6 | 7.6e−01 | 0.5 |
| stomach | 3.7e−01 | 3.2e−01 | 1 | 0.1 | 1 | 0.3 |
| uterus | 9.4e−01 | 7.0e−01 | 1 | 0.6 | 4.1e−01 | 1.1 |

As noted above, cluster HSS100PCB features 1 transcript(s), which were listed in Table 1 above. These transcript(s) encode for protein(s) which are variant(s) of protein S-100P protein (SEQ ID NO:635). A description of each variant protein according to the present invention is now provided.

Variant protein HSS100PCB_P3 (SEQ ID NO:580) according to the present invention has an amino acid sequence as given at the end of the application; it is encoded by transcript(s) HSS100PCB_T1 (SEQ ID NO:49). The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSS100PCB_P3 (SEQ ID NO:580) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein HSS100PCB_P3 (SEQ ID NO:580) sequence provides support for the deduced sequence of this variant protein according to the present invention).

TABLE 7
Amino acid mutations

| SNP position(s) on amino | | Previously known |
|---|---|---|
| acid sequence | Alternative amino acid(s) | SNP? |
| 1 | M -> R | Yes |
| 11 | M -> L | Yes |
| 20 | L -> F | Yes |

Variant protein HSS100PCB_P3 (SEQ ID NO:580) is encoded by the following transcript(s): HSS100PCB_T1 (SEQ ID NO:49), for which the sequence(s) is/are given at the end of the application. The coding portion of transcript HSS100PCB_T1 (SEQ ID NO:49) is shown in bold; this coding portion starts at position 1057 and ends at position 1533. The transcript also has the following SNPs as listed in Table 8 (given according to their position on the nucleotide sequence, with the alternative nucleic acid listed; the last column indicates whether the SNP is known or not; the presence of known SNPs in variant protein HSS100PCB_P3 (SEQ ID NO:580) sequence provides support for the deduced sequence of this variant protein according to the present invention).

TABLE 8
Nucleic acid SNPs

| SNP position on nucleotide | | Previously known |
|---|---|---|
| sequence | Alternative nucleic acid | SNP? |
| 52 | C -> T | Yes |
| 107 | A -> C | Yes |
| 458 | C -> T | Yes |
| 468 | A -> G | Yes |
| 648 | C -> T | Yes |
| 846 | C -> G | Yes |
| 882 | G -> A | Yes |
| 960 | C -> T | No |
| 965 | C -> T | Yes |
| 1058 | T -> G | Yes |
| 1087 | A -> C | Yes |
| 1114 | C -> T | Yes |
| 1968 | G -> A | Yes |
| 1971 | C -> T | Yes |
| 2010 | C -> A | Yes |
| 2099 | G -> | No |

As noted above, cluster HSS100PCB features 3 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HSS100PCB_node_{—}3 (SEQ ID NO:363) according to the present invention is supported by 16 libraries. The number of libraries was determined as previously described. This segment can be found in the following transcript(s): HSS100PCB_T1 (SEQ ID NO:49). Table 9 below describes the starting and ending position of this segment on each transcript.

TABLE 9
Segment location on transcripts

| | Segment | Segment |
|---|---|---|
| Transcript name | starting position | ending position |
| HSS100PCB_T1 (SEQ ID NO: 49) | 1 | 1133 |

Segment cluster HSS100PCB_node_{—}4 (SEQ ID NO:364) according to the present invention is supported by 29 libraries. The number of libraries was determined as previously described. This segment can be found in the following transcript(s): HSS100PCB_T1 (SEQ ID NO:49). Table 10 below describes the starting and ending position of this segment on each transcript.

TABLE 10
Segment location on transcripts

| | Segment | Segment |
|---|---|---|
| Transcript name | starting position | ending position |
| HSS100PCB_T1 (SEQ ID NO: 49) | 1134 | 1923 |

Microarray (chip) data is also available for this segment as follows. As described above with regard to the cluster itself, various oligonucleotides were tested for being differentially expressed in various disease conditions, particularly cancer. The following oligonucleotides were found to hit this segment (related to colon cancer), shown in Table 11.

TABLE 11
Oligonucleotides related to this segment

| Oligonucleotide name | Overexpressed in cancers | Chip reference |
|---|---|---|
| HSS100PCB_0_0_12280 | colorectal cancer | Colon |
| (SEQ ID NO: 1413) | | |

Segment cluster HSS100PCB_node_{—}5 (SEQ ID NO:365) according to the present invention is supported by 141 libraries. The number of libraries was determined as previously described. This segment can be found in the following transcript(s): HSS100PCB_T1 (SEQ ID NO:49). Table 12 below describes the starting and ending position of this segment on each transcript.

TABLE 12
Segment location on transcripts

| | Segment | Segment |
|---|---|---|
| Transcript name | starting position | ending position |
| HSS100PCB_T1 (SEQ ID NO: 49) | 1924 | 2201 |

### Description for Cluster HUMPHOSLIP

Cluster HUMPHOSLIP features 7 transcript(s) and 53 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| HUMPHOSLIP_PEA_2_T6 | 50 |
| HUMPHOSLIP_PEA_2_T7 | 51 |
| HUMPHOSLIP_PEA_2_T14 | 52 |
| HUMPHOSLIP_PEA_2_T16 | 53 |
| HUMPHOSLIP_PEA_2_T17 | 54 |
| HUMPHOSLIP_PEA_2_T18 | 55 |
| HUMPHOSLIP_PEA_2_T19 | 56 |

TABLE 2
Segments of interest

| Segment Name | SEQ ID NO: |
|---|---|
| HUMPHOSLIP_PEA_2_node_0 | 366 |
| HUMPHOSLIP_PEA_2_node_19 | 367 |
| HUMPHOSLIP_PEA_2_node_34 | 368 |
| HUMPHOSLIP_PEA_2_node_68 | 369 |
| HUMPHOSLIP_PEA_2_node_70 | 370 |
| HUMPHOSLIP_PEA_2_node_75 | 371 |
| HUMPHOSLIP_PEA_2_node_2 | 372 |
| HUMPHOSLIP_PEA_2_node_3 | 373 |
| HUMPHOSLIP_PEA_2_node_4 | 374 |
| HUMPHOSLIP_PEA_2_node_6 | 375 |
| HUMPHOSLIP_PEA_2_node_7 | 376 |
| HUMPHOSLIP_PEA_2_node_8 | 377 |
| HUMPHOSLIP_PEA_2_node_9 | 378 |
| HUMPHOSLIP_PEA_2_node_14 | 379 |
| HUMPHOSLIP_PEA_2_node_15 | 380 |
| HUMPHOSLIP_PEA_2_node_16 | 381 |
| HUMPHOSLIP_PEA_2_node_17 | 382 |
| HUMPHOSLIP_PEA_2_node_23 | 383 |
| HUMPHOSLIP_PEA_2_node_24 | 384 |
| HUMPHOSLIP_PEA_2_node_25 | 385 |
| HUMPHOSLIP_PEA_2_node_26 | 386 |
| HUMPHOSLIP_PEA_2_node_29 | 387 |
| HUMPHOSLIP_PEA_2_node_30 | 388 |
| HUMPHOSLIP_PEA_2_node_33 | 389 |
| HUMPHOSLIP_PEA_2_node_36 | 390 |
| HUMPHOSLIP_PEA_2_node_37 | 391 |
| HUMPHOSLIP_PEA_2_node_39 | 392 |
| HUMPHOSLIP_PEA_2_node_40 | 393 |
| HUMPHOSLIP_PEA_2_node_41 | 394 |
| HUMPHOSLIP_PEA_2_node_42 | 395 |
| HUMPHOSLIP_PEA_2_node_44 | 396 |
| HUMPHOSLIP_PEA_2_node_45 | 397 |
| HUMPHOSLIP_PEA_2_node_47 | 398 |
| HUMPHOSLIP_PEA_2_node_51 | 399 |
| HUMPHOSLIP_PEA_2_node_52 | 400 |
| HUMPHOSLIP_PEA_2_node_53 | 401 |
| HUMPHOSLIP_PEA_2_node_54 | 402 |
| HUMPHOSLIP_PEA_2_node_55 | 403 |
| HUMPHOSLIP_PEA_2_node_58 | 404 |
| HUMPHOSLIP_PEA_2_node_59 | 405 |
| HUMPHOSLIP_PEA_2_node_60 | 406 |
| HUMPHOSLIP_PEA_2_node_61 | 407 |
| HUMPHOSLIP_PEA_2_node_62 | 408 |
| HUMPHOSLIP_PEA_2_node_63 | 409 |
| HUMPHOSLIP_PEA_2_node_64 | 410 |
| HUMPHOSLIP_PEA_2_node_65 | 411 |
| HUMPHOSLIP_PEA_2_node_66 | 412 |
| HUMPHOSLIP_PEA_2_node_67 | 413 |
| HUMPHOSLIP_PEA_2_node_69 | 414 |
| HUMPHOSLIP_PEA_2_node_71 | 415 |
| HUMPHOSLIP_PEA_2_node_72 | 416 |
| HUMPHOSLIP_PEA_2_node_73 | 417 |
| HUMPHOSLIP_PEA_2_node_74 | 418 |

TABLE 3
Proteins of interest

| | SEQ | |
|---|---|---|
| | ID | |
| Protein Name | NO: | Corresponding Transcript(s) |
| HUMPHOSLIP_PEA_2_P10 | 581 | HUMPHOSLIP_PEA_2_T17 |
| | | (SEQ ID NO: 54) |
| HUMPHOSLIP_PEA_2_P12 | 582 | HUMPHOSLIP_PEA_2_T19 |
| | | (SEQ ID NO: 56) |
| HUMPHOSLIP_PEA_2_P30 | 583 | HUMPHOSLIP_PEA_2_T6 |
| | | (SEQ ID NO: 50) |
| HUMPHOSLIP_PEA_2_P31 | 584 | HUMPHOSLIP_PEA_2_T7 |
| | | (SEQ ID NO: 51) |
| HUMPHOSLIP_PEA_2_P33 | 585 | HUMPHOSLIP_PEA_2_T14 |
| | | (SEQ ID NO: 52) |
| HUMPHOSLIP_PEA_2_P34 | 586 | HUMPHOSLIP_PEA_2_T16 |
| | | (SEQ ID NO: 53) |
| HUMPHOSLIP_PEA_2_P35 | 587 | HUMPHOSLIP_PEA_2_T18 |
| | | (SEQ ID NO: 55) |

These sequences are variants of the known protein Phospholipid transfer protein precursor (SwissProt accession identifier PLTP_HUMAN; known also according to the synonyms Lipid transfer protein II), SEQ ID NO: 636, referred to herein as the previously known protein.

Protein Phospholipid transfer protein precursor (SEQ ID NO:636) is known or believed to have the following function(s): Converts HDL into larger and smaller particles. May play a key role in extracellular phospholipid transport and modulation of hdl particles. The sequence for protein Phospholipid transfer protein precursor is given at the end of the application, as “Phospholipid transfer protein precursor amino acid sequence”. Known polymorphisms for this sequence are as shown in Table 4.

TABLE 4
Amino acid mutations for Known Protein

| SNP position(s) | |
|---|---|
| on amino | |
| acid sequence | Comment |
| 282 | R -> Q. /FTId = VAR_017020. |
| 372 | R -> H. /FTId = VAR_017021. |
| 380 | R -> W (in dbSNP: 6065903). /FTId = VAR_017022. |
| 444 | F -> L (in dbSNP: 1804161). /FTId = VAR_012073. |
| 487 | T -> K (in dbSNP: 1056929). /FTId = VAR_012074. |
| 18 | E -> V |

Protein Phospholipid transfer protein precursor (SEQ ID NO:636) localization is believed to be secreted.

The following GO Annotation(s) apply to the previously known protein. The following annotation(s) were found: lipid metabolism; lipid transport, which are annotation(s) related to Biological Process; lipid binding, which are annotation(s) related to Molecular Function; and extracellular, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

For this cluster, at least one oligonucleotide was found to demonstrate overexpression of the cluster, although not of at least one transcript/segment as listed below. Microarray (chip) data is also available for this cluster as follows. Various oligonucleotides were tested for being differentially expressed in various disease conditions, particularly cancer, as previously described. The following oligonucleotides were found to hit this cluster (in relation to colon cancer) but not other segments/transcripts below, shown in Table 5.

TABLE 5
Oligonucleotides related to this cluster

| Oligonucleotide name | Overexpressed in cancers | Chip reference |
|---|---|---|
| HUMPHOSLIP_0_0_18458 | colorectal cancer | Colon |
| (SEQ ID NO: 1414) | | |

AS noted above, cluster HUMPHOSLIP features/transcript(s), which were listed in Table 1 above. These transcript(s) encode for protein(s) which are variant(s) of protein Phospholipid transfer protein precursor (SEQ ID NO:636). A description of each variant protein according to the present invention is now provided.

Variant protein HUMPHOSLIP_PEA_{—}2_P10 (SEQ ID NO:581) according to the present invention has an amino acid sequence as given at the end of the application; it is encoded by transcript(s) HUMPHOSLIP_PEA_{—}2_T17 (SEQ ID NO:54). An alignment is given to the known protein (Phospholipid transfer protein precursor (SEQ ID NO:636)) at the end of the application. One or more alignments to one or more previously published protein sequences are given at the end of the application. A brief description of the relationship of the variant protein according to the present invention to each such aligned protein is as follows:

Comparison Report Between HUMPHOSLIP_PEA_{—}2_P10 (SEQ ID NO:581) and PLTP_HUMAN (SEQ ID NO:636):

1. An isolated chimeric polypeptide encoding for HUMPHOSLIP_PEA_{—}2_P10 (SEQ ID NO:581) comprising a first amino acid sequence being at least 90% homologous to MALFGALFLALLAGAHAEFPGCKIRVTSKALELVKQEGLRFLEQELETITIPDLRGKEGBIFYYNISE corresponding to amino acids 1-67 of PLTP_HUMAN (SEQ ID NO:636), which also corresponds to amino acids 1-67 of HUMPHOSLIP_PEA_{—}2_P10 (SEQ ID NO:581), and a second amino acid sequence being at least 90% homologous to KVYDFLSTFITSGMRFLLNQQICPVLYHAGTVLLNSLLDTVPVRSSVDELVGIDYSLMKDPVASTSNLDMD FRGAFFPLTERNWSLPNRAVEPQLQEEERMVYVAFSEFFFDSAMESYFRAGALQLLLVGDKVPHDLDMLL RATYFGSIVLLSPAVIDSPLKLELRVLAPPRCTIKPSGTTISVTASVTIALVPPDQPEVQLSSMTMDARLSAK MALRGKALRTQLDLRRFRIYSNHSALESLALIPLQAPLKTMLQIGVMPMLNERTWRGVQIPLPEGINFVHE VVTNHAGFLTIGADLHFAKGLREVIEKNRPADVRASTAPTPSTAAV corresponding to amino acids 163-493 of PLTP_HUMAN (SEQ ID NO:636), which also corresponds to amino acids 68-398 of HUMPHOSLIP_PEA_{—}2_P10 (SEQ ID NO:581), wherein said first amino acid sequence and second amino acid sequence are contiguous and in a sequential order.

2. An isolated chimeric polypeptide encoding for an edge portion of HUMPHOSLIP_PEA_{—}2_P10 (SEQ ID NO:581), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EK, having a structure as follows: a sequence starting from any of amino acid numbers 67−x to 67; and ending at any of amino acid numbers 68+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMPHOSLIP_PEA_{—}2_P10 (SEQ ID NO:581) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein HUMPHOSLIP_PEA_{—}2_P10 (SEQ ID NO:581) sequence provides support for the deduced sequence of this variant protein according to the present invention).

TABLE 6
Amino acid mutations

| SNP position(s) on | Alternative | Previously |
|---|---|---|
| amino acid sequence | amino acid(s) | known SNP? |
| 16 | H -> R | Yes |
| 18 | E -> V | Yes |
| 113 | S -> F | Yes |
| 118 | V -> | No |
| 140 | R -> | No |
| 140 | R -> P | No |
| 150 | N -> | No |
| 160 | P -> | No |
| 201 | P -> | No |
| 274 | M -> | No |
| 285 | R -> W | Yes |
| 292 | Q -> | No |
| 315 | L -> * | No |
| 330 | M -> I | Yes |
| 349 | F -> L | Yes |
| 392 | T -> K | Yes |

The glycosylation sites of variant protein HUMPHOSLIP_PEA_{—}2_P10 (SEQ ID NO:581), as compared to the known protein Phospholipid transfer protein precursor (SEQ ID NO:636), are described in Table 7 (given according to their position(s) on the amino acid sequence in the first column; the second column indicates whether the glycosylation site is present in the variant protein; and the last column indicates whether the position is different on the variant protein).

TABLE 7
Glycosylation site(s)

| Position(s) on known | Precent in | Position in |
|---|---|---|
| amino acid sequence | variant protein? | variant protein? |
| 94 | No | |
| 143 | No | |
| 64 | Yes | 64 |
| 245 | Yes | 150 |
| 398 | Yes | 303 |
| 117 | No | |

Variant protein HUMPHOSLIP_PEA_{—}2_P10 (SEQ ID NO:581) is encoded by the following transcript(s): HUMPHOSLIP_PEA_{—}2_T17 (SEQ ID NO:54), for which the sequence(s) is/are given at the end of the application. The coding portion of transcript HUMPHOSLIP_PEA_{—}2_T17 (SEQ ID NO:54) is shown in bold; this coding portion starts at position 276 and ends at position 1469. The transcript also has the following SNPs as listed in Table 8 (given according to their position on the nucleotide sequence, with the alternative nucleic acid listed; the last column indicates whether the SNP is known or not; the presence of known SNPs in variant protein HUMPHOSLIP_PEA_{—}2_P10 (SEQ ID NO:581) sequence provides support for the deduced sequence of this variant protein according to the present invention).

TABLE 8
Nucleic acid SNPs

| SNP position on | Alternative | Previously |
|---|---|---|
| nucleotide sequence | nucleic acid | known SNP? |
| 174 | G -> T | No |
| 175 | A -> T | No |
| 322 | A -> G | Yes |
| 328 | A -> T | Yes |
| 431 | G -> A | Yes |
| 551 | C -> T | Yes |
| 613 | C -> T | Yes |
| 628 | T -> | No |
| 694 | G -> | No |
| 694 | G -> C | No |
| 723 | A -> | No |
| 753 | C -> | No |
| 876 | C -> | No |
| 1037 | C -> T | Yes |
| 1097 | G -> | No |
| 1128 | C -> T | Yes |
| 1149 | C -> | No |
| 1219 | T -> A | No |
| 1230 | C -> T | Yes |
| 1265 | G -> C | Yes |
| 1322 | T -> A | Yes |
| 1450 | C -> A | Yes |
| 1469 | C -> T | No |
| 1549 | C -> T | Yes |
| 1565 | A -> G | No |
| 1565 | A -> T | No |
| 1630 | A -> G | Yes |
| 1654 | T -> A | No |
| 1731 | G -> T | Yes |
| 1864 | G -> A | Yes |
| 1893 | G -> T | Yes |
| 2073 | G -> A | Yes |
| 2269 | C -> T | Yes |
| 2325 | G -> T | Yes |
| 2465 | C -> T | Yes |
| 2566 | C -> T | Yes |
| 2881 | A -> G | No |

Variant protein HUMPHOSLIP_PEA_{—}2_P12 (SEQ ID NO:582) according to the present invention has an amino acid sequence as given at the end of the application; it is encoded by transcript(s) HUMPHOSLIP_PEA_{—}2_T19 (SEQ ID NO:56). An alignment is given to the known protein (Phospholipid transfer protein precursor (SEQ ID NO:636)) at the end of the application. One or more alignments to one or more previously published protein sequences are given at the end of the application. A brief description of the relationship of the variant protein according to the present invention to each such aligned protein is as follows:

Comparison Report Between HUMPHOSLIP_PEA_{—}2_P12 (SEQ ID NO:582) and PLTP_HUMAN (SEQ ID NO:636):

1. An isolated chimeric polypeptide encoding for HUMPHOSLIP_PEA_{—}2_P12 (SEQ ID NO:582) comprising a first amino acid sequence being at least 90% homologous to MALFGALFLALLAGAHAEFPGCKIRVTSKALELVKQEGLRFLEQELETITIPDLRGKEGHFYYNISEVKVTE LQLTSSELDFQPQQELMLQITNASLGLRFRRQLLYWFFYDGGYINASAEGVSIRTGLELSRDPAGRMKVSN VSCQASVSRMHAAFGGTFKKVYDFLSTFITSGMRFLLNQQICPVLYHAGTVLLNSLLDTVPVRSSVDELVG IDYSLMKDPVASTSNLDMDFRGAFFPLTERNWSLPNRAVEPQLQEEERMVYVAFSEFFFDSAMESYFRAG ALQLLLVGDKVPHDLDMLLRATYFGSIVLLSPAVIDSPLKLELRVLAPPRCTIKPSGTTISVTASVTIALVPP DQPEVQLSSMTMDARLSAKMALRGKALRTQLDLRRFRIYSNHSALESLALIPLQAPLKTMLQIGVMPMLN corresponding to amino acids 1-427 of PLTP_HUMAN (SEQ ID NO:636), which also corresponds to amino acids 1-427 of HUMPHOSLIP_PEA_{—}2_P12 (SEQ ID NO:582), and a second amino acid sequence being at least 70%, optionally at least 80%, preferably at least 85%, more preferably at least 90% and most preferably at least 95% homologous to a polypeptide having the sequence GKAGV (SEQ ID NO:1525) corresponding to amino acids 428-432 of HUMPHOSLIP_PEA_{—}2_P12 (SEQ ID NO:582), wherein said first amino acid sequence and second amino acid sequence are contiguous and in a sequential order.

2. An isolated polypeptide encoding for a tail of HUMPHOSLIP_PEA_{—}2_P12 (SEQ ID NO:582) comprising a polypeptide being at least 70%, optionally at least about 80%, preferably at least about 85%, more preferably at least about 90% and most preferably at least about 95% homologous to the sequence GKAGV (SEQ ID NO:1525) in HUMPHOSLIP_PEA_{—}2_P12 (SEQ ID NO:582).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMPHOSLIP_PEA_{—}2_P12 (SEQ ID NO:582) also has the following non-silent SNPs (Single Nucleotide Polymorphisms) as listed in Table 9, (given according to their position(s) on the amino acid sequence, with the alternative amino acid(s) listed; the last column indicates whether the SNP is known or not; the presence of known SNPs in variant protein HUMPHOSLIP_PEA_{—}2_P12 (SEQ ID NO:582) sequence provides support for the deduced sequence of this variant protein according to the present invention).

TABLE 9
Amino acid mutations

| SNP position(s) on | Alternative | Previously |
|---|---|---|
| amino acid sequence | amino acid(s) | known SNP? |
| 16 | H -> R | Yes |
| 18 | E -> V | Yes |
| 81 | D -> H | Yes |
| 124 | S -> Y | Yes |
| 160 | T -> | No |
| 160 | T -> N | No |
| 208 | S -> F | Yes |
| 213 | V -> | No |
| 235 | R -> P | No |
| 235 | R -> | No |
| 245 | N -> | No |
| 255 | P -> | No |
| 296 | P -> | No |
| 369 | M -> | No |
| 380 | R -> W | Yes |
| 387 | Q -> | No |
| 410 | L -> * | No |
| 425 | M -> I | Yes |

The glycosylation sites of variant protein HUMPHOSLIP_PEA_{—}2_P12 (SEQ ID NO:582), as compared to the known protein Phospholipid transfer protein precursor (SEQ ID NO:636), are described in Table 10 (given according to their position(s) on the amino acid sequence in the first column; the second column indicates whether the glycosylation site is present in the variant protein; and the last column indicates whether the position is different on the variant protein).

TABLE 10
Glycosylation site(s)

| Position(s) on known | Present in | Position in |
|---|---|---|
| amino acid sequence | variant protein? | variant protein? |
| 94 | Yes | 94 |
| 143 | Yes | 143 |
| 64 | Yes | 64 |
| 245 | Yes | 245 |
| 398 | Yes | 398 |
| 117 | Yes | 117 |

Variant protein HUMPHOSLIP_PEA_{—}2_P12 (SEQ ID NO:582) is encoded by the following transcript(s): HUMPHOSLIP_PEA_{—}2_T19 (SEQ ID NO:56), for which the sequence(s) is/are given at the end of the application. The coding portion of transcript HUMPHOSLIP_PEA_{—}2_T19 (SEQ ID NO:56) is shown in bold; this coding portion starts at position 276 and ends at position 1571. The transcript also has the following SNPs as listed in Table 11 (given according to their position on the nucleotide sequence, with the alternative nucleic acid listed; the last column indicates whether the SNP is known or not; the presence of known SNPs in variant protein HUMPHOSLIP_PEA_{—}2_P12 (SEQ ID NO:582) sequence provides support for the deduced sequence of this variant protein according to the present invention).

TABLE 11
Nucleic acid SNPs

| SNP position on | Alternative | Previously |
|---|---|---|
| nucleotide sequence | nucleic acid | known SNP? |
| 174 | G -> T | No |
| 175 | A -> T | No |
| 322 | A -> G | Yes |
| 328 | A -> T | Yes |
| 431 | G -> A | Yes |
| 516 | G -> C | Yes |
| 644 | G -> A | Yes |
| 646 | C -> A | Yes |
| 754 | C -> | No |
| 754 | C -> A | No |
| 836 | C -> T | Yes |
| 898 | C -> T | Yes |
| 913 | T -> | No |
| 979 | G -> | No |
| 979 | G -> C | No |
| 1008 | A -> | No |
| 1038 | C -> | No |
| 1161 | C -> | No |
| 1322 | C -> T | Yes |
| 1382 | G -> | No |
| 1413 | C -> T | Yes |
| 1434 | C -> | No |
| 1504 | T -> A | No |
| 1515 | C -> T | Yes |
| 1550 | G -> C | Yes |
| 1690 | T -> A | Yes |
| 1818 | C -> A | Yes |
| 1837 | C -> T | No |
| 1917 | C -> T | Yes |
| 1933 | A -> G | No |
| 1933 | A -> T | No |
| 1998 | A -> G | Yes |
| 2022 | T -> A | No |
| 2099 | G -> T | Yes |
| 2232 | G -> A | Yes |
| 2261 | G -> T | Yes |
| 2441 | G -> A | Yes |
| 2637 | C -> T | Yes |
| 2693 | G -> T | Yes |
| 2833 | C -> T | Yes |
| 2934 | C -> T | Yes |
| 3249 | A -> G | No |

Variant protein HUMPHOSLIP_PEA_{—}2_P30 (SEQ ID NO:583) according to the present invention has an amino acid sequence as given at the end of the application; it is encoded by transcript(s) HUMPHOSLIP_PEA_{—}2_T6 (SEQ ID NO:50). The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMPHOSLIP_PEA_{—}2_P30 (SEQ ID NO:583) also has the following non-silent SNPs (Single Nucleotide Polymorphisms) as listed in Table 12, (given according to their position(s) on the amino acid sequence, with the alternative amino acid(s) listed; the last column indicates whether the SNP is known or not; the presence of known SNPs in variant protein HUMPHOSLIP_PEA_{—}2_P30 (SEQ ID NO:583) sequence provides support for the deduced sequence of this variant protein according to the present invention).

TABLE 12
Amino acid mutations

| SNP position(s) on | Alternative | Previously |
|---|---|---|
| amino acid sequence | amino acid(s) | known SNP? |
| 16 | H -> R | Yes |
| 18 | E -> V | Yes |
| 37 | R -> Q | Yes |

Variant protein HUMPHOSLIP_PEA_{—}2_P30 (SEQ ID NO:583) is encoded by the following transcript(s): HUMPHOSLIP_PEA_{—}2_T6 (SEQ ID NO:50), for which the sequence(s) is/are given at the end of the application. The coding portion of transcript HUMPHOSLIP_PEA_{—}2_T6 (SEQ ID NO:50) is shown in bold; this coding portion starts at position 276 and ends at position 431. The transcript also has the following SNPs as listed in Table 13 (given according to their position on the nucleotide sequence, with the alternative nucleic acid listed; the last column indicates whether the SNP is known or not; the presence of known SNPs in variant protein HUMPHOSLIP_PEA_{—}2_P30 (SEQ ID NO:583) sequence provides support for the deduced sequence of this variant protein according to the present invention).

TABLE 13
Nucleic acid SNPs

| SNP position on | Alternative | Previously |
|---|---|---|
| nucleotide sequence | nucleic acid | known SNP? |
| 174 | G -> T | No |
| 175 | A -> T | No |
| 322 | A -> G | Yes |
| 328 | A -> T | Yes |
| 385 | G -> A | Yes |
| 470 | G -> C | Yes |
| 598 | G -> A | Yes |
| 600 | C -> A | Yes |
| 708 | C -> | No |
| 708 | C -> A | No |
| 790 | C -> T | Yes |
| 852 | C -> T | Yes |
| 867 | T -> | No |
| 933 | G -> | No |
| 933 | G -> C | No |
| 962 | A -> | No |
| 992 | C -> | No |
| 1115 | C -> | No |
| 1276 | C -> T | Yes |
| 1336 | G -> | No |
| 1367 | C -> T | Yes |
| 1388 | C -> | No |
| 1458 | T -> A | No |
| 1469 | C -> T | Yes |
| 1504 | G -> C | Yes |
| 1561 | T -> A | Yes |
| 1689 | C -> A | Yes |
| 1708 | C -> T | No |
| 1788 | C -> T | Yes |
| 1804 | A -> G | No |
| 1804 | A -> T | No |
| 1869 | A -> G | Yes |
| 1893 | T -> A | No |
| 1970 | G -> T | Yes |
| 2103 | G -> A | Yes |
| 2132 | G -> T | Yes |
| 2312 | G -> A | Yes |
| 2508 | C -> T | Yes |
| 2564 | G -> T | Yes |
| 2704 | C -> T | Yes |
| 2805 | C -> T | Yes |
| 3120 | A -> G | No |

Variant protein HUMPHOSLIP_PEA_{—}2_P31 (SEQ ID NO:584) according to the present invention has an amino acid sequence as given at the end of the application; it is encoded by transcript(s) HUMPHOSLIP_PEA_{—}2_T7 (SEQ ID NO:51). An alignment is given to the known protein (Phospholipid transfer protein precursor (SEQ ID NO:636)) at the end of the application. One or more alignments to one or more previously published protein sequences are given at the end of the application. A brief description of the relationship of the variant protein according to the present invention to each such aligned protein is as follows:

Comparison Report Between HUMPHOSLIP_PEA_{—}2_P31 (SEQ ID NO:584) and PLTP_HUMAN (SEQ ID NO:636):

1. An isolated chimeric polypeptide encoding for HUMPHOSLIP_PEA_{—}2_P31 (SEQ ID NO:584) comprising a first amino acid sequence being at least 90% homologous to MALFGALFLALLAGAHAEFPGCKIRVTSKALELVKQEGLRFLEQELETITIPDLRGKEGHFYYNISE corresponding to amino acids 1-67 of PLTP_HUMAN (SEQ ID NO:636), which also corresponds to amino acids 1-67 of HUMPHOSLIP_PEA_{—}2_P31 (SEQ ID NO:584), and a second amino acid sequence being at least 70%, optionally at least 80%, preferably at least 85%, more preferably at least 90% and most preferably at least 95% homologous to a polypeptide having the sequence PGLERGADKFPVVGGSSLFLALDLTLRPPVG (SEQ ID NO:1526) corresponding to amino acids 68-98 of HUMPHOSLIP_PEA_{—}2_P31 (SEQ ID NO:584), wherein said first amino acid sequence and second amino acid sequence are contiguous and in a sequential order.

2. An isolated polypeptide encoding for a tail of HUMPHOSLIP_PEA_{—}2_P31 (SEQ ID NO:584) comprising a polypeptide being at least 70%, optionally at least about 80%, preferably at least about 85%, more preferably at least about 90% and most preferably at least about 95% homologous to the sequence PGLERGADKFPVVGGSSLFLALDLTLRPPVG (SEQ ID NO:1526) in HUMPHOSLIP_PEA_{—}2_P31 (SEQ ID NO:584).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMPHOSLIP_PEA_{—}2_P31 (SEQ ID NO:584) also has the following non-silent SNPs (Single Nucleotide Polymorphisms) as listed in Table 14, (given according to their position(s) on the amino acid sequence, with the alternative amino acid(s) listed; the last column indicates whether the SNP is known or not; the presence of known SNPs in variant protein HUMPHOSLIP_PEA_{—}2_P31 (SEQ ID NO:584) sequence provides support for the deduced sequence of this variant protein according to the present invention).

TABLE 14
Amino acid mutations

| SNP position(s) on | Alternative | Previously |
|---|---|---|
| amino acid sequence | amino acid(s) | known SNP? |
| 16 | H -> R | Yes |
| 18 | E -> V | Yes |

The glycosylation sites of variant protein HUMPHOSLIP_PEA_{—}2_P31 (SEQ ID NO:584), as compared to the known protein Phospholipid transfer protein precursor (SEQ ID NO:636), are described in Table 15 (given according to their position(s) on the amino acid sequence in the first column; the second column indicates whether the glycosylation site is present in the variant protein; and the last column indicates whether the position is different on the variant protein).

TABLE 15
Glycosylation site(s)

| Position(s) on known | Present in | Position in |
|---|---|---|
| amino acid sequence | variant protein? | variant protein? |
| 94 | No | |
| 143 | No | |
| 64 | Yes | 64 |
| 245 | No | |
| 398 | No | |
| 117 | No | |

Variant protein HUMPHOSLIP_PEA_{—}2_P31 (SEQ ID NO:584) is encoded by the following transcript(s): HUMPHOSLIP_PEA_{—}2_T7 (SEQ ID NO:51), for which the sequence(s) is/are given at the end of the application. The coding portion of transcript HUMPHOSLIP_PEA_{—}2_T7 (SEQ ID NO:51) is shown in bold; this coding portion starts at position 276 and ends at position 569. The transcript also has the following SNPs as listed in Table 16 (given according to their position on the nucleotide sequence, with the alternative nucleic acid listed; the last column indicates whether the SNP is known or not; the presence of known SNPs in variant protein HUMPHOSLIP_PEA_{—}2_P31 (SEQ ID NO:584) sequence provides support for the deduced sequence of this variant protein according to the present invention).

TABLE 16
Nucleic acid SNPs

| SNP position on | Alternative | Previously |
|---|---|---|
| nucleotide sequence | nucleic acid | known SNP? |
| 174 | G -> T | No |
| 175 | A -> T | No |
| 322 | A -> G | Yes |
| 328 | A -> T | Yes |
| 431 | G -> A | Yes |
| 608 | G -> C | Yes |
| 736 | G -> A | Yes |
| 738 | C -> A | Yes |
| 846 | C -> | No |
| 846 | C -> A | No |
| 928 | C -> T | Yes |
| 990 | C -> T | Yes |
| 1005 | T -> | No |
| 1071 | G -> | No |
| 1071 | G -> C | No |
| 1100 | A -> | No |
| 1130 | C -> | No |
| 1253 | C -> | No |
| 1414 | C -> T | Yes |
| 1474 | G -> | No |
| 1505 | C -> T | Yes |
| 1526 | C -> | No |
| 1596 | T -> A | No |
| 1607 | C -> T | Yes |
| 1642 | G -> C | Yes |
| 1699 | T -> A | Yes |
| 1827 | C -> A | Yes |
| 1846 | C -> T | No |
| 1926 | C -> T | Yes |
| 1942 | A -> G | No |
| 1942 | A -> T | No |
| 2007 | A -> G | Yes |
| 2031 | T -> A | No |
| 2108 | G -> T | Yes |
| 2241 | G -> A | Yes |
| 2270 | G -> T | Yes |
| 2450 | G -> A | Yes |
| 2646 | C -> T | Yes |
| 2702 | G -> T | Yes |
| 2842 | C -> T | Yes |
| 2943 | C -> T | Yes |
| 3258 | A -> G | No |

Variant protein HUMPHOSLIP_PEA_{—}2_P33 (SEQ ID NO:585) according to the present invention has an amino acid sequence as given at the end of the application; it is encoded by transcript(s) HUMPHOSLIP_PEA_{—}2_T14 (SEQ ID NO:52). An alignment is given to the known protein (Phospholipid transfer protein precursor (SEQ ID NO:636)) at the end of the application. One or more alignments to one or more previously published protein sequences are given at the end of the application. A brief description of the relationship of the variant protein according to the present invention to each such aligned protein is as follows:

Comparison Report Between HUMPHOSLIP_PEA_{—}2_P33 (SEQ ID NO:585) and PLTP_HUMAN (SEQ ID NO:636):

1. An isolated chimeric polypeptide encoding for HUMPHOSLIP_PEA_{—}2_P33 (SEQ ID NO:585) comprising a first amino acid sequence being at least 90% homologous to MALFGALFLALLAGAHAEFPGCKIRVTSKALELVKQEGLRFLEQELETITIPDLRGKEGHFYYNISEVKVTE LQLTSSELDFQPQQELMLQITNASLGLRFRRQLLYWFFYDGGYINASAEGVSIRTGLELSRDPAGRMKVSN VSCQASVSRMHAAFGGTFKKVYDFLSTFITSGMRFLLNQQ corresponding to amino acids 1-183 of PLTP_HUMAN (SEQ ID NO:636), which also corresponds to amino acids 1-183 of HUMPHOSLIP_PEA_{—}2_P33 (SEQ ID NO:585), and a second amino acid sequence being at least 70%, optionally at least 80%, preferably at least 85%, more preferably at least 90% and most preferably at least 95% homologous to a polypeptide having the sequence VWAATGRRVARVGMLSL (SEQ ID NO:1527) corresponding to amino acids 184-200 of HUMPHOSLIP_PEA_{—}2_P33 (SEQ ID NO:585), wherein said first amino acid sequence and second amino acid sequence are contiguous and in a sequential order.

2. An isolated polypeptide encoding for a tail of HUMPHOSLIP_PEA_{—}2_P33 (SEQ ID NO:585) comprising a polypeptide being at least 70%, optionally at least about 80%, preferably at least about 85%, more preferably at least about 90% and most preferably at least about 95% homologous to the sequence VWAATGRRVARVGMLSL (SEQ ID NO:1527) in HUMPHOSLIP_PEA_{—}2_P33 (SEQ ID NO:585).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMPHOSLIP_PEA_{—}2_P33 (SEQ ID NO:585) also has the following non-silent SNPs (Single Nucleotide Polymorphisms) as listed in Table 17, (given according to their position(s) on the amino acid sequence, with the alternative amino acid(s) listed; the last column indicates whether the SNP is known or not; the presence of known SNPs in variant protein HUMPHOSLIP_PEA_{—}2_P33 (SEQ ID NO:585) sequence provides support for the deduced sequence of this variant protein according to the present invention).

TABLE 17
Amino acid mutations

| SNP position(s) on | Alternative | Previously |
|---|---|---|
| amino acid sequence | amino acid(s) | known SNP? |
| 16 | H -> R | Yes |
| 18 | E -> V | Yes |
| 81 | D -> H | Yes |
| 124 | S -> Y | Yes |
| 160 | T -> | No |
| 160 | T -> N | No |

The glycosylation sites of variant protein HUMPHOSLIP_PEA_{—}2_P33 (SEQ ID NO:585), as compared to the known protein Phospholipid transfer protein precursor (SEQ ID NO:636), are described in Table 18 (given according to their position(s) on the amino acid sequence in the first column; the second column indicates whether the glycosylation site is present in the variant protein; and the last column indicates whether the position is different on the variant protein).

TABLE 18
Glycosylation site(s)

| Position(s) on known | Present in | Position in |
|---|---|---|
| amino acid sequence | variant protein? | variant protein? |
| 94 | Yes | 94 |
| 143 | Yes | 143 |
| 64 | Yes | 64 |
| 245 | No | |
| 398 | No | |
| 117 | Yes | 117 |

Variant protein HUMPHOSLIP_PEA_{—}2_P33 (SEQ ID NO:585) is encoded by the following transcript(s): HUMPHOSLIP_PEA_{—}2_T14 (SEQ ID NO:52), for which the sequence(s) is/are given at the end of the application. The coding portion of transcript HUMPHOSLIP_PEA_{—}2_T14 (SEQ ID NO:52) is shown in bold; this coding portion starts at position 276 and ends at position 875. The transcript also has the following SNPs as listed in Table 19 (given according to their position on the nucleotide sequence, with the alternative nucleic acid listed; the last column indicates whether the SNP is known or not; the presence of known SNPs in variant protein HUMPHOSLIP_PEA_{—}2_P33 (SEQ ID NO:585) sequence provides support for the deduced sequence of this variant protein according to the present invention).

TABLE 19
Nucleic acid SNPs

| SNP position on | Alternative | Previously |
|---|---|---|
| nucleotide sequence | nucleic acid | known SNP? |
| 174 | G -> T | No |
| 175 | A -> T | No |
| 322 | A -> G | Yes |
| 328 | A -> T | Yes |
| 431 | G -> A | Yes |
| 516 | G -> C | Yes |
| 644 | G -> A | Yes |
| 646 | C -> A | Yes |
| 754 | C -> | No |
| 754 | C -> A | No |
| 921 | C -> T | Yes |
| 983 | C -> T | Yes |
| 998 | T -> | No |
| 1064 | G -> | No |
| 1064 | G -> C | No |
| 1093 | A -> | No |
| 1123 | C -> | No |
| 1246 | C -> | No |
| 1407 | C -> T | Yes |
| 1467 | G -> | No |
| 1498 | C -> T | Yes |
| 1519 | C -> | No |
| 1589 | T -> A | No |
| 1600 | C -> T | Yes |
| 1635 | G -> C | Yes |
| 1692 | T -> A | Yes |
| 1820 | C -> A | Yes |
| 1839 | C -> T | No |
| 1919 | C -> T | Yes |
| 1935 | A -> G | No |
| 1935 | A -> T | No |
| 2000 | A -> G | Yes |
| 2024 | T -> A | No |
| 2101 | G -> T | Yes |
| 2234 | G -> A | Yes |
| 2263 | G -> T | Yes |
| 2443 | G -> A | Yes |
| 2639 | C -> T | Yes |
| 2695 | G -> T | Yes |
| 2835 | C -> T | Yes |
| 2936 | C -> T | Yes |
| 3251 | A -> G | No |

Variant protein HUMPHOSLIP_PEA_{—}2_P34 (SEQ ID NO:586) according to the present invention has an amino acid sequence as given at the end of the application; it is encoded by transcript(s) HUMPHOSLIP_PEA_{—}2_T16 (SEQ ID NO:53). An alignment is given to the known protein (Phospholipid transfer protein precursor (SEQ ID NO:636)) at the end of the application. One or more alignments to one or more previously published protein sequences are given at the end of the application. A brief description of the relationship of the variant protein according to the present invention to each such aligned protein is as follows:

Comparison Report Between HUMPHOSLIP_PEA_{—}2_P34 (SEQ ID NO:586) and PLTP_HUMAN (SEQ ID NO:636):

1. An isolated chimeric polypeptide encoding for HUMPHOSLIP_PEA_{—}2_P34 (SEQ ID NO:586) comprising a first amino acid sequence being at least 90% homologous to MALFGALFLALLAGAHAEFPGCKIRVTSKALELVKQEGLRFLEQELETITIPDLRGKEGHFYYNISEVKVTE LQLTSSELDFQPQQELMLQITNASLGLRFRRQLLYWFFYDGGYINASAEGVSIRTGLELSRDPAGRMKVSN VSCQASVSRMHAAFGGTFKKVYDFLSTFITSGMRFLLNQQICPVLYHAGTVLLNSLLDTVPV corresponding to amino acids 1-205 of PLTP_HUMAN (SEQ ID NO:636), which also corresponds to amino acids 1-205 of HUMPHOSLIP_PEA_{—}2_P34 (SEQ ID NO:586), and a second amino acid sequence being at least 70%, optionally at least 80%, preferably at least 85%, more preferably at least 90% and most preferably at least 95% homologous to a polypeptide having the sequence LWTSLLALTIPS (SEQ ID NO:1528) corresponding to amino acids 206-217 of HUMPHOSLIP_PEA_{—}2_P34 (SEQ ID NO:586), wherein said first amino acid sequence and second amino acid sequence are contiguous and in a sequential order.

2. An isolated polypeptide encoding for a tail of HUMPHOSLIP_PEA_{—}2_P34 (SEQ ID NO:586) comprising a polypeptide being at least 70%, optionally at least about 80%, preferably at least about 85%, more preferably at least about 90% and most preferably at least about 95% homologous to the sequence LWTSLLALTIPS (SEQ ID NO:1528) in HUMPHOSLIP_PEA_{—}2_P34 (SEQ ID NO:586).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMPHOSLIP_PEA_{—}2_P34 (SEQ ID NO:586) also has the following non-silent SNPs (Single Nucleotide Polymorphisms) as listed in Table 20, (given according to their position(s) on the amino acid sequence, with the alternative amino acid(s) listed; the last column indicates whether the SNP is known or not; the presence of known SNPs in variant protein HUMPHOSLIP_PEA_{—}2_P34 (SEQ ID NO:586) sequence provides support for the deduced sequence of this variant protein according to the present invention).

TABLE 20
Amino acid mutations

| SNP position(s) on | Alternative | Previously |
|---|---|---|
| amino acid sequence | amino acid(s) | kn own SNP? |
| 16 | H -> R | Yes |
| 18 | E -> V | Yes |
| 81 | D -> H | Yes |
| 124 | S -> Y | Yes |
| 160 | T -> | No |
| 160 | T -> N | No |
| 211 | L -> | No |

The glycosylation sites of variant protein HUMPHOSLIP_PEA_{—}2_P34 (SEQ ID NO:586), as compared to the known protein Phospholipid transfer protein precursor (SEQ ID NO:636), are described in Table 21 (given according to their position(s) on the amino acid sequence in the first column; the second column indicates whether the glycosylation site is present in the variant protein; and the last column indicates whether the position is different on the variant protein).

TABLE 21
Glycosylation site(s)

| Position(s) on known | Present in | Position in |
|---|---|---|
| amino acid sequence | variant protein? | variant protein? |
| 94 | Yes | 94 |
| 143 | Yes | 143 |
| 64 | Yes | 64 |
| 245 | No | |
| 398 | No | |
| 117 | Yes | 117 |

Variant protein HUMPHOSLIP_PEA_{—}2_P34 (SEQ ID NO:586) is encoded by the following transcript(s): HUMPHOSLIP_PEA_{—}2_T16 (SEQ ID NO:53), for which the sequence(s) is/are given at the end of the application. The coding portion of transcript HUMPHOSLIP_PEA_{—}2_T16 (SEQ ID NO:53) is shown in bold; this coding portion starts at position 276 and ends at position 926. The transcript also has the following SNPs as listed in Table 22 (given according to their position on the nucleotide sequence, with the alternative nucleic acid listed; the last column indicates whether the SNP is known or not; the presence of known SNPs in variant protein HUMPHOSLIP_PEA_{—}2_P34 (SEQ ID NO:586) sequence provides support for the deduced sequence of this variant protein according to the present invention).

TABLE 22
Nucleic acid SNPs

| SNP position on | Alternative | Previously |
|---|---|---|
| nucleotide sequence | nucleic acid | known SNP? |
| 174 | G -> T | No |
| 175 | A -> T | No |
| 322 | A -> G | Yes |
| 328 | A -> T | Yes |
| 431 | G -> A | Yes |
| 516 | G -> C | Yes |
| 644 | G -> A | Yes |
| 646 | C -> A | Yes |
| 754 | C -> | No |
| 754 | C -> A | No |
| 836 | C -> T | Yes |
| 891 | C -> T | Yes |
| 906 | T -> | No |
| 972 | G -> | No |
| 972 | G -> C | No |
| 1001 | A -> | No |
| 1031 | C -> | No |
| 1154 | C -> | No |
| 1315 | C -> T | Yes |
| 1375 | G -> | No |
| 1406 | C -> T | Yes |
| 1427 | C -> | No |
| 1497 | T -> A | No |
| 1508 | C -> T | Yes |
| 1543 | G -> C | Yes |
| 1600 | T -> A | Yes |
| 1728 | C -> A | Yes |
| 1747 | C -> T | No |
| 1827 | C -> T | Yes |
| 1843 | A -> G | No |
| 1843 | A -> T | No |
| 1908 | A -> G | Yes |
| 1932 | T -> A | No |
| 2009 | G -> T | Yes |
| 2142 | G -> A | Yes |
| 2171 | G -> T | Yes |
| 2351 | G -> A | Yes |
| 2547 | C -> T | Yes |
| 2603 | G -> T | Yes |
| 2743 | C -> T | Yes |
| 2844 | C -> T | Yes |
| 3159 | A -> G | No |

Variant protein HUMPHOSLIP_PEA_{—}2_P35 (SEQ ID NO:587) according to the present invention has an amino acid sequence as given at the end of the application; it is encoded by transcript(s) HUMPHOSLIP_PEA_{—}2_T18 (SEQ ID NO:55). An alignment is given to the known protein (Phospholipid transfer protein precursor (SEQ ID NO:636)) at the end of the application. One or more alignments to one or more previously published protein sequences are given at the end of the application. A brief description of the relationship of the variant protein according to the present invention to each such aligned protein is as follows:

Comparison Report Between HUMPHOSLIP_PEA_{—}2_P35 (SEQ ID NO:587) and PLTP_HUMAN (SEQ ID NO:636):

1. An isolated chimeric polypeptide encoding for HUMPHOSLIP_PEA_{—}2_P35 (SEQ ID NO:587) comprising a first amino acid sequence being at least 90% homologous to MALFGALFLALLAGAHAEFPGCKIRVTSKALELVKQEGLRFLEQELETITIPDLRGKEGHFYYNISEVKVTE LQLTSSELDFQPQQELMLQITNASLGLRFRRQLLYWF corresponding to amino acids 1-109 of PLTP_HUMAN (SEQ ID NO:636), which also corresponds to amino acids 1-109 of HUMPHOSLIP_PEA_{—}2_P35 (SEQ ID NO:587), a second amino acid sequence bridging amino acid sequence comprising of L, a third amino acid sequence being at least 90% homologous to KVYDFLSTFITSGMRFLLNQQ corresponding to amino acids 163-183 of PLTP_HUMAN (SEQ ID NO:636), which also corresponds to amino acids 111-131 of HUMPHOSLIP_PEA_{—}2_P35 (SEQ ID NO:587), and a fourth amino acid sequence being at least 70%, optionally at least 80%, preferably at least 85%, more preferably at least 90% and most preferably at least 95% homologous to a polypeptide having the sequence VWAATGRRVARVGMLSL (SEQ ID NO:1527) corresponding to amino acids 132-148 of HUMPHOSLIP_PEA_{—}2_P35 (SEQ ID NO:587), wherein said first amino acid sequence, second amino acid sequence, third amino acid sequence and fourth amino acid sequence are contiguous and in a sequential order.

2. An isolated polypeptide encoding for an edge portion of HUMPHOSLIP_PEA_{—}2_P35 (SEQ ID NO:587), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise

FLK having a structure as follows (numbering according to HUMPHOSLIP_PEA_{—}2_P35 (SEQ ID NO:587)): a sequence starting from any of amino acid numbers 109−x to 109; and ending at any of amino acid numbers 111+((n−2)−x), in which x varies from 0 to n−2.

3. An isolated polypeptide encoding for a tail of HUMPHOSLIP_PEA_{—}2_P35 (SEQ ID NO:587) comprising a polypeptide being at least 70%, optionally at least about 80%, preferably at least about 85%, more preferably at least about 90% and most preferably at least about 95% homologous to the sequence VWAATGRRVARVGMLSL (SEQ ID NO:1527) in HUMPHOSLIP_PEA_{—}2_P35 (SEQ ID NO:587).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMPHOSLIP_PEA_{—}2_P35 (SEQ ID NO:587) also has the following non-silent SNPs (Single Nucleotide Polymorphisms) as listed in Table 23, (given according to their position(s) on the amino acid sequence, with the alternative amino acid(s) listed; the last column indicates whether the SNP is known or not; the presence of known SNPs in variant protein HUMPHOSLIP_PEA_{—}2_P35 (SEQ ID NO:587) sequence provides support for the deduced sequence of this variant protein according to the present invention).

TABLE 23
Amino acid mutations

| SNP position(s) on | Alternative | Previously |
|---|---|---|
| amino acid sequence | amino acid(s) | known SNP? |
| 16 | H -> R | Yes |
| 18 | E -> V | Yes |
| 81 | D -> H | Yes |

The glycosylation sites of variant protein HUMPHOSLIP_PEA_{—}2_P35 (SEQ ID NO:587), as compared to the known protein Phospholipid transfer protein precursor (SEQ ID NO:636), are described in Table 24 (given according to their position(s) on the amino acid sequence in the first column; the second column indicates whether the glycosylation site is present in the variant protein; and the last column indicates whether the position is different on the variant protein).

TABLE 24
Glycosylation site(s)

| Position(s) on known | Present in | Position in |
|---|---|---|
| amino acid sequence | variant protein? | variant protein? |
| 94 | Yes | 94 |
| 143 | No | |
| 64 | Yes | 64 |
| 245 | No | |
| 398 | No | |
| 117 | No | |

Variant protein HUMPHOSLIP_PEA_{—}2_P35 (SEQ ID NO:587) is encoded by the following transcript(s): HUMPHOSLIP_PEA_{—}2_T18 (SEQ ID NO:55), for which the sequence(s) is/are given at the end of the application. The coding portion of transcript HUMPHOSLIP_PEA_{—}2_T18 (SEQ ID NO:55) is shown in bold; this coding portion starts at position 276 and ends at position 719. The transcript also has the following SNPs as listed in Table 25 (given according to their position on the nucleotide sequence, with the alternative nucleic acid listed; the last column indicates whether the SNP is known or not; the presence of known SNPs in variant protein HUMPHOSLIP_PEA_{—}2_P35 (SEQ ID NO:587) sequence provides support for the deduced sequence of this variant protein according to the present invention).

TABLE 25
Nucleic acid SNPs

| SNP position on | Alternative | Previously |
|---|---|---|
| nucleotide sequence | nucleic acid | known SNP? |
| 174 | G -> T | No |
| 175 | A -> T | No |
| 322 | A -> G | Yes |
| 328 | A -> T | Yes |
| 431 | G -> A | Yes |
| 516 | G -> C | Yes |
| 765 | C -> T | Yes |
| 827 | C -> T | Yes |
| 842 | T -> | No |
| 908 | G -> | No |
| 908 | G -> C | No |
| 937 | A -> | No |
| 967 | C -> | No |
| 1090 | C -> | No |
| 1251 | C -> T | Yes |
| 1311 | G -> | No |
| 1342 | C -> T | Yes |
| 1363 | C -> | No |
| 1433 | T -> A | No |
| 1444 | C -> T | Yes |
| 1479 | G -> C | Yes |
| 1536 | T -> A | Yes |
| 1664 | C -> A | Yes |
| 1683 | C -> T | No |
| 1763 | C -> T | Yes |
| 1779 | A -> G | No |
| 1779 | A -> T | No |
| 1844 | A -> G | Yes |
| 1868 | T -> A | No |
| 1945 | G -> T | Yes |
| 2078 | G -> A | Yes |
| 2107 | G -> T | Yes |
| 2287 | G -> A | Yes |
| 2483 | C -> T | Yes |
| 2539 | G -> T | Yes |
| 2679 | C -> T | Yes |
| 2780 | C -> T | Yes |
| 3095 | A -> G | No |

As noted above, cluster HUMPHOSLIP features 53 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HUMPHOSLIP_PEA_{—}2_node_{—}0 (SEQ ID NO:366) according to the present invention is supported by 150 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 26 below describes the starting and ending position of this segment on each transcript.

TABLE 26
Segment location on transcripts

| | Segment | Segment |
|---|---|---|
| | starting | ending |
| Transcript name | position | position |
| HUMPHOSLIP_PEA_2_T6 (SEQ ID NO: 50) | 1 | 264 |
| HUMPHOSLIP_PEA_2_T7 (SEQ ID NO: 51) | 1 | 264 |
| HUMPHOSLIP_PEA_2_T14 (SEQ ID NO: 52) | 1 | 264 |
| HUMPHOSLIP_PEA_2_T16 (SEQ ID NO: 53) | 1 | 264 |
| HUMPHOSLIP_PEA_2_T17 (SEQ ID NO: 54) | 1 | 264 |
| HUMPHOSLIP_PEA_2_T18 (SEQ ID NO: 55) | 1 | 264 |
| HUMPHOSLIP_PEA_2_T19 (SEQ ID NO: 56) | 1 | 264 |

Segment cluster HUMPHOSLIP_PEA_{—}2_node_{—}19 (SEQ ID NO:367) according to the present invention is supported by 186 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53) and HUMPHOSLIP_PEA_{—}2_T19 (SEQ ID NO:56). Table 27 below describes the starting and ending position of this segment on each transcript.

TABLE 27
Segment location on transcripts

| | Segment | Segment |
|---|---|---|
| | starting | ending |
| Transcript name | position | position |
| HUMPHOSLIP_PEA_2_T6 (SEQ ID NO: 50) | 559 | 714 |
| HUMPHOSLIP_PEA_2_T7 (SEQ ID NO: 51) | 697 | 852 |
| HUMPHOSLIP_PEA_2_T14 (SEQ ID NO: 52) | 605 | 760 |
| HUMPHOSLIP_PEA_2_T16 (SEQ ID NO: 53) | 605 | 760 |
| HUMPHOSLIP_PEA_2_T19 (SEQ ID NO: 56) | 605 | 760 |

Segment cluster HUMPHOSLIP_PEA_{—}2_node_{—}34 (SEQ ID NO:368) according to the present invention is supported by 191 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 28 below describes the starting and ending position of this segment on each transcript.

TABLE 28
Segment location on transcripts

| | Segment | Segment |
|---|---|---|
| | starting | ending |
| Transcript name | position | position |
| HUMPHOSLIP_PEA_2_T6 (SEQ ID NO: 50) | 971 | 1111 |
| HUMPHOSLIP_PEA_2_T7 (SEQ ID NO: 51) | 1109 | 1249 |
| HUMPHOSLIP_PEA_2_T14 (SEQ ID NO: 52) | 1102 | 1242 |
| HUMPHOSLIP_PEA_2_T16 (SEQ ID NO: 53) | 1010 | 1150 |
| HUMPHOSLIP_PEA_2_T17 (SEQ ID NO: 54) | 732 | 872 |
| HUMPHOSLIP_PEA_2_T18 (SEQ ID NO: 55) | 946 | 1086 |
| HUMPHOSLIP_PEA_2_T19 (SEQ ID NO: 56) | 1017 | 1157 |

Segment cluster HUMPHOSLIP_PEA_{—}2_node_{—}68 (SEQ ID NO:369) according to the present invention is supported by 131 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 29 below describes the starting and ending position of this segment on each transcript.

TABLE 29
Segment location on transcripts

| | Segment | Segment |
|---|---|---|
| | starting | ending |
| Transcript name | position | position |
| HUMPHOSLIP_PEA_2_T6 (SEQ ID NO: 50) | 1867 | 2285 |
| HUMPHOSLIP_PEA_2_T7 (SEQ ID NO: 51) | 2005 | 2423 |
| HUMPHOSLIP_PEA_2_T14 (SEQ ID NO: 52) | 1998 | 2416 |
| HUMPHOSLIP_PEA_2_T16 (SEQ ID NO: 53) | 1906 | 2324 |
| HUMPHOSLIP_PEA_2_T17 (SEQ ID NO: 54) | 1628 | 2046 |
| HUMPHOSLIP_PEA_2_T18 (SEQ ID NO: 55) | 1842 | 2260 |
| HUMPHOSLIP_PEA_2_T19 (SEQ ID NO: 56) | 1996 | 2414 |

Segment cluster HUMPHOSLIP_PEA_{—}2_node_{—}70 (SEQ ID NO:370) according to the present invention is supported by 5 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 30 below describes the starting and ending position of this segment on each transcript.

TABLE 30
Segment location on transcripts

| | Segment | Segment |
|---|---|---|
| | starting | ending |
| Transcript name | position | position |
| HUMPHOSLIP_PEA_2_T6 (SEQ ID NO: 50) | 2298 | 2529 |
| HUMPHOSLIP_PEA_2_T7 (SEQ ID NO: 51) | 2436 | 2667 |
| HUMPHOSLIP_PEA_2_T14 (SEQ ID NO: 52) | 2429 | 2660 |
| HUMPHOSLIP_PEA_2_T16 (SEQ ID NO: 53) | 2337 | 2568 |
| HUMPHOSLIP_PEA_2_T17 (SEQ ID NO: 54) | 2059 | 2290 |
| HUMPHOSLIP_PEA_2_T18 (SEQ ID NO: 55) | 2273 | 2504 |
| HUMPHOSLIP_PEA_2_T19 (SEQ ID NO: 56) | 2427 | 2658 |

Segment cluster HUMPHOSLIP_PEA_{—}2_node_{—}75 (SEQ ID NO:371) according to the present invention is supported by 14 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 31 below describes the starting and ending position of this segment on each transcript.

TABLE 31
Segment location on transcripts

| | Segment | Segment |
|---|---|---|
| | starting | ending |
| Transcript name | position | position |
| HUMPHOSLIP_PEA_2_T6 (SEQ ID NO: 50) | 2846 | 3125 |
| HUMPHOSLIP_PEA_2_T7 (SEQ ID NO: 51) | 2984 | 3263 |
| HUMPHOSLIP_PEA_2_T14 (SEQ ID NO: 52) | 2977 | 3256 |
| HUMPHOSLIP_PEA_2_T16 (SEQ ID NO: 53) | 2885 | 3164 |
| HUMPHOSLIP_PEA_2_T17 (SEQ ID NO: 54) | 2607 | 2886 |
| HUMPHOSLIP_PEA_2_T18 (SEQ ID NO: 55) | 2821 | 3100 |
| HUMPHOSLIP_PEA_2_T19 (SEQ ID NO: 56) | 2975 | 3254 |

According to an optional embodiment of the present invention, short segments related to the above cluster are also provided. These segments are up to about 120 bp in length, and so are included in a separate description.

Segment cluster HUMPHOSLIP_PEA_{—}2_node_{—}2 (SEQ ID NO:372) according to the present invention is supported by 159 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 32 below describes the starting and ending position of this segment on each transcript.

TABLE 32
Segment location on transcripts

| | Segment | Segment |
|---|---|---|
| | starting | ending |
| Transcript name | position | position |
| HUMPHOSLIP_PEA_2_T6 (SEQ ID NO: 50) | 265 | 337 |
| HUMPHOSLIP_PEA_2_T7 (SEQ ID NO: 51) | 265 | 337 |
| HUMPHOSLIP_PEA_2_T14 (SEQ ID NO: 52) | 265 | 337 |
| HUMPHOSLIP_PEA_2_T16 (SEQ ID NO: 53) | 265 | 337 |
| HUMPHOSLIP_PEA_2_T17 (SEQ ID NO: 54) | 265 | 337 |
| HUMPHOSLIP_PEA_2_T18 (SEQ ID NO: 55) | 265 | 337 |
| HUMPHOSLIP_PEA_2_T19 (SEQ ID NO: 56) | 265 | 337 |

Segment cluster HUMPHOSLIP_PEA_{—}2_node_{—}3 (SEQ ID NO:373) according to the present invention can be found in the following transcript(s): HUMPHOSLIP_PEA_{—}2_T7 (SEQ ID NO:51) (SEQ ID NO:52), HUMPHOSLIP_PEA_{—}2_T16 (SEQ ID NO:53) (SEQ ID NO:54), HUMPHOSLIP_PEA_{—}2_T18 (SEQ ID NO:55) and HUMPHOSLIP_PEA_{—}2_T19 (SEQ ID NO:56). Table 33 below describes the starting and ending position of this segment on each transcript.

TABLE 33
Segment location on transcripts

| | Segment | Segment |
|---|---|---|
| | starting | ending |
| Transcript name | position | position |
| HUMPHOSLIP_PEA_2_T7 (SEQ ID NO: 51) | 338 | 355 |
| HUMPHOSLIP_PEA_2_T14 (SEQ ID NO: 52) | 338 | 355 |
| HUMPHOSLIP_PEA_2_T16 (SEQ ID NO: 53) | 338 | 355 |
| HUMPHOSLIP_PEA_2_T17 (SEQ ID NO: 54) | 338 | 355 |
| HUMPHOSLIP_PEA_2_T18 (SEQ ID NO: 55) | 338 | 355 |
| HUMPHOSLIP_PEA_2_T19 (SEQ ID NO: 56) | 338 | 355 |

Segment cluster HUMPHOSLIP_PEA_{—}2_node_{—}4 (SEQ ID NO:374) according to the present invention can be found in the following transcript(s): HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 34 below describes the starting and ending position of this segment on each transcript.

TABLE 34
Segment location on transcripts

| | Segment | Segment |
|---|---|---|
| | starting | ending |
| Transcript name | position | position |
| HUMPHOSLIP_PEA_2_T7 (SEQ ID NO: 51) | 356 | 375 |
| HUMPHOSLIP_PEA_2_T14 (SEQ ID NO: 52) | 356 | 375 |
| HUMPHOSLIP_PEA_2_T16 (SEQ ID NO: 53) | 356 | 375 |
| HUMPHOSLIP_PEA_2_T17 (SEQ ID NO: 54) | 356 | 375 |
| HUMPHOSLIP_PEA_2_T18 (SEQ ID NO: 55) | 356 | 375 |
| HUMPHOSLIP_PEA_2_T19 (SEQ ID NO: 56) | 356 | 375 |

Segment cluster HUMPHOSLIP_PEA_{—}2_node_{—}6 (SEQ ID NO:375) according to the present invention can be found in the following transcript(s): HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 35 below describes the starting and ending position of this segment on each transcript.

TABLE 35
Segment location on transcripts

| | Segment | Segment |
|---|---|---|
| | starting | ending |
| Transcript name | position | position |
| HUMPHOSLIP_PEA_2_T7 (SEQ ID NO: 51) | 376 | 383 |
| HUMPHOSLIP_PEA_2_T14 (SEQ ID NO: 52) | 376 | 383 |
| HUMPHOSLIP_PEA_2_T16 (SEQ ID NO: 53) | 376 | 383 |
| HUMPHOSLIP_PEA_2_T17 (SEQ ID NO: 54) | 376 | 383 |
| HUMPHOSLIP_PEA_2_T18 (SEQ ID NO: 55) | 376 | 383 |
| HUMPHOSLIP_PEA_2_T19 (SEQ ID NO: 56) | 376 | 383 |

Segment cluster HUMPHOSLIP_PEA_{—}2_node_{—}7 (SEQ ID NO:376) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 36 below describes the starting and ending position of this segment on each transcript.

TABLE 36
Segment location on transcripts

| | Segment | Segment |
|---|---|---|
| | starting | ending |
| Transcript name | position | position |
| HUMPHOSLIP_PEA_2_T6 (SEQ ID NO: 50) | 338 | 343 |
| HUMPHOSLIP_PEA_2_T7 (SEQ ID NO: 51) | 384 | 389 |
| HUMPHOSLIP_PEA_2_T14 (SEQ ID NO: 52) | 384 | 389 |
| HUMPHOSLIP_PEA_2_T16 (SEQ ID NO: 53) | 384 | 389 |
| HUMPHOSLIP_PEA_2_T17 (SEQ ID NO: 54) | 384 | 389 |
| HUMPHOSLIP_PEA_2_T18 (SEQ ID NO: 55) | 384 | 389 |
| HUMPHOSLIP_PEA_2_T19 (SEQ ID NO: 56) | 384 | 389 |

Segment cluster HUMPHOSLIP_PEA_{—}2_node_{—}8 (SEQ ID NO:377) according to the present invention is supported by 171 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 37 below describes the starting and ending position of this segment on each transcript.

TABLE 37
Segment location on transcripts

| | Segment | Segment |
|---|---|---|
| | starting | ending |
| Transcript name | position | position |
| HUMPHOSLIP_PEA_2_T6 (SEQ ID NO: 50) | 334 | 378 |
| HUMPHOSLIP_PEA_2_T7 (SEQ ID NO: 51) | 390 | 424 |
| HUMPHOSLIP_PEA_2_T14 (SEQ ID NO: 52) | 390 | 424 |
| HUMPHOSLIP_PEA_2_T16 (SEQ ID NO: 53) | 390 | 424 |
| HUMPHOSLIP_PEA_2_T17 (SEQ ID NO: 54) | 390 | 424 |
| HUMPHOSLIP_PEA_2_T18 (SEQ ID NO: 55) | 390 | 424 |
| HUMPHOSLIP_PEA_2_T19 (SEQ ID NO: 56) | 390 | 424 |

Segment cluster HUMPHOSLIP_PEA_{—}2_node_{—}9 (SEQ ID NO:378) according to the present invention is supported by 168 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 38 below describes the starting and ending position of this segment on each transcript.

TABLE 38
Segment location on transcripts

| | Segment | Segment |
|---|---|---|
| | starting | ending |
| Transcript name | position | position |
| HUMPHOSLIP_PEA_2_T6 (SEQ ID NO: 50) | 379 | 429 |
| HUMPHOSLIP_PEA_2_T7 (SEQ ID NO: 51) | 425 | 475 |
| HUMPHOSLIP_PEA_2_T14 (SEQ ID NO: 52) | 425 | 475 |
| HUMPHOSLIP_PEA_2_T16 (SEQ ID NO: 53) | 425 | 475 |
| HUMPHOSLIP_PEA_2_T17 (SEQ ID NO: 54) | 425 | 475 |
| HUMPHOSLIP_PEA_2_T18 (SEQ ID NO: 55) | 425 | 475 |
| HUMPHOSLIP_PEA_2_T19 (SEQ ID NO: 56) | 425 | 475 |

Segment cluster HUMPHOSLIP_PEA_{—}2_node_{—}14 (SEQ ID NO:379) according to the present invention is supported by 6 libraries. The number of libraries was determined as previously described. This segment can be found in the following transcript(s): HUMPHOSLIP_PEA_{—}2_T7 (SEQ ID NO:51). Table 39 below describes the starting and ending position of this segment on each transcript.

TABLE 39
Segment location on transcripts

| | Segment | Segment |
|---|---|---|
| | starting | ending |
| Transcript name | position | position |
| HUMPHOSLIP_PEA_2_T7 (SEQ ID NO: 51) | 476 | 567 |

Segment cluster HUMPHOSLIP_PEA_{—}2_node_{—}15 (SEQ ID NO:380) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T18 (SEQ ID NO:55) and HUMPHOSLIP_PEA_{—}2_T19 (SEQ ID NO:56). Table 40 below describes the starting and ending position of this segment on each transcript.

TABLE 40
Segment location on transcripts

| | Segment | Segment |
|---|---|---|
| | starting | ending |
| Transcript name | position | position |
| HUMPHOSLIP_PEA_2_T6 (SEQ ID NO: 50) | 430 | 445 |
| HUMPHOSLIP_PEA_2_T7 (SEQ ID NO: 51) | 568 | 583 |
| HUMPHOSLIP_PEA_2_T14 (SEQ ID NO: 52) | 476 | 491 |
| HUMPHOSLIP_PEA_2_T16 (SEQ ID NO: 53) | 476 | 491 |
| HUMPHOSLIP_PEA_2_T18 (SEQ ID NO: 55) | 476 | 491 |
| HUMPHOSLIP_PEA_2_T19 (SEQ ID NO: 56) | 476 | 491 |

Segment cluster HUMPHOSLIP_PEA_{—}2_node_{—}16 (SEQ ID NO:381) according to the present invention is supported by 179 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T18 (SEQ ID NO:55) and HUMPHOSLIP_PEA_{—}2_T19 (SEQ ID NO:56). Table 41 below describes the starting and ending position of this segment on each transcript.

TABLE 41
Segment location on transcripts

| | Segment | Segment |
|---|---|---|
| | starting | ending |
| Transcript name | position | position |
| HUMPHOSLIP_PEA_2_T6 (SEQ ID NO: 50) | 446 | 534 |
| HUMPHOSLIP_PEA_2_T7 (SEQ ID NO: 51) | 584 | 672 |
| HUMPHOSLIP_PEA_2_T14 (SEQ ID NO: 52) | 492 | 580 |
| HUMPHOSLIP_PEA_2_T16 (SEQ ID NO: 53) | 492 | 580 |
| HUMPHOSLIP_PEA_2_T18 (SEQ ID NO: 55) | 492 | 580 |
| HUMPHOSLIP_PEA_2_T19 (SEQ ID NO: 56) | 492 | 580 |

Segment cluster HUMPHOSLIP_PEA_{—}2_node_{—}17 (SEQ ID NO:382) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T18 (SEQ ID NO:55) and HUMPHOSLIP_PEA_{—}2_T19 (SEQ ID NO:56). Table 42 below describes the starting and ending position of this segment on each transcript.

TABLE 42
Segment location on transcripts

| | Segment | Segment |
|---|---|---|
| | starting | ending |
| Transcript name | position | position |
| HUMPHOSLIP_PEA_2_T6 (SEQ ID NO: 50) | 535 | 558 |
| HUMPHOSLIP_PEA_2_T7 (SEQ ID NO: 51) | 673 | 696 |
| HUMPHOSLIP_PEA_2_T14 (SEQ ID NO: 52) | 581 | 604 |
| HUMPHOSLIP_PEA_2_T16 (SEQ ID NO: 53) | 581 | 604 |
| HUMPHOSLIP_PEA_2_T18 (SEQ ID NO: 55) | 581 | 604 |
| HUMPHOSLIP_PEA_2_T19 (SEQ ID NO: 56) | 581 | 604 |

Segment cluster HUMPHOSLIP_PEA_{—}2_node_{—}23 (SEQ ID NO:383) according to the present invention is supported by 168 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 43 below describes the starting and ending position of this segment on each transcript.

TABLE 43
Segment location on transcripts

| | Segment | Segment |
|---|---|---|
| | starting | ending |
| Transcript name | position | position |
| HUMPHOSLIP_PEA_2_T6 (SEQ ID NO: 50) | 715 | 766 |
| HUMPHOSLIP_PEA_2_T7 (SEQ ID NO: 51) | 853 | 904 |
| HUMPHOSLIP_PEA_2_T14 (SEQ ID NO: 52) | 761 | 812 |
| HUMPHOSLIP_PEA_2_T16 (SEQ ID NO: 53) | 761 | 812 |
| HUMPHOSLIP_PEA_2_T17 (SEQ ID NO: 54) | 476 | 527 |
| HUMPHOSLIP_PEA_2_T18 (SEQ ID NO: 55) | 605 | 656 |
| HUMPHOSLIP_PEA_2_T19 (SEQ ID NO: 56) | 761 | 812 |

Segment cluster HUMPHOSLIP_PEA_{—}2_node_{—}24 (SEQ ID NO:384) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 44 below describes the starting and ending position of this segment on each transcript.

TABLE 44
Segment location on transcripts

| | Segment | Segment |
|---|---|---|
| | starting | ending |
| Transcript name | position | position |
| HUMPHOSLIP_PEA_2_T6 (SEQ ID NO: 50) | 767 | 778 |
| HUMPHOSLIP_PEA_2_T7 (SEQ ID NO: 51) | 905 | 916 |
| HUMPHOSLIP_PEA_2_T14 (SEQ ID NO: 52) | 813 | 824 |
| HUMPHOSLIP_PEA_2_T16 (SEQ ID NO: 53) | 813 | 824 |
| HUMPHOSLIP_PEA_2_T17 (SEQ ID NO: 54) | 528 | 539 |
| HUMPHOSLIP_PEA_2_T18 (SEQ ID NO: 55) | 657 | 668 |
| HUMPHOSLIP_PEA_2_T19 (SEQ ID NO: 56) | 813 | 824 |

Segment cluster HUMPHOSLIP_PEA_{—}2_node_{—}25 (SEQ ID NO:385) according to the present invention is supported by 5 libraries. The number of libraries was determined as previously described. This segment can be found in the following transcript(s): HUMPHOSLIP_PEA_{—}2_T14 (SEQ ID NO:52) and HUMPHOSLIP_PEA_{—}2_T18 (SEQ ID NO:55). Table 45 below describes the starting and ending position of this segment on each transcript.

TABLE 45
Segment location on transcripts

| | Segment | Segment |
|---|---|---|
| | starting | ending |
| Transcript name | position | position |
| HUMPHOSLIP_PEA_2_T14 (SEQ ID NO: 52) | 825 | 909 |
| HUMPHOSLIP_PEA_2_T18 (SEQ ID NO: 55) | 669 | 753 |

Segment cluster HUMPHOSLIP_PEA_{—}2_node_{—}26 (SEQ ID NO:386) according to the present invention is supported by 163 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 46 below describes the starting and ending position of this segment on each transcript.

TABLE 46
Segment location on transcripts

| | Segment | Segment |
|---|---|---|
| | starting | ending |
| Transcript name | position | position |
| HUMPHOSLIP_PEA_2_T6 (SEQ ID NO: 50) | 779 | 842 |
| HUMPHOSLIP_PEA_2_T7 (SEQ ID NO: 51) | 917 | 980 |
| HUMPHOSLIP_PEA_2_T14 (SEQ ID NO: 52) | 910 | 973 |
| HUMPHOSLIP_PEA_2_T16 (SEQ ID NO: 53) | 825 | 888 |
| HUMPHOSLIP_PEA_2_T17 (SEQ ID NO: 54) | 540 | 603 |
| HUMPHOSLIP_PEA_2_T18 (SEQ ID NO: 55) | 754 | 817 |
| HUMPHOSLIP_PEA_2_T19 (SEQ ID NO: 56) | 825 | 888 |

Segment cluster HUMPHOSLIP_PEA_{—}2_node_{—}29 (SEQ ID NO:387) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 47 below describes the starting and ending position of this segment on each transcript.

TABLE 47
Segment location on transcripts

| | Segment | Segment |
|---|---|---|
| | starting | ending |
| Transcript name | position | position |
| HUMPHOSLIP_PEA_2_T6 (SEQ ID NO: 50) | 843 | 849 |
| HUMPHOSLIP_PEA_2_T7 (SEQ ID NO: 51) | 981 | 387 |
| HUMPHOSLIP_PEA_2_T14 (SEQ ID NO: 52) | 974 | 980 |
| HUMPHOSLIP_PEA_2_T17 (SEQ ID NO: 54) | 604 | 610 |
| HUMPHOSLIP_PEA_2_T18 (SEQ ID NO: 55) | 818 | 824 |
| HUMPHOSLIP_PEA_2_T19 (SEQ ID NO: 56) | 889 | 895 |

Segment cluster HUMPHOSLIP_PEA_{—}2_node_{—}30 (SEQ ID NO:388) according to the present invention is supported by 181 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 48 below describes the starting and ending position of this segment on each transcript.

TABLE 48
Segment location on transcripts

| | Segment | Segment |
|---|---|---|
| | starting | ending |
| Transcript name | position | position |
| HUMPHOSLIP_PEA_2_T6 (SEQ ID NO: 50) | 850 | 934 |
| HUMPHOSLIP_PEA_2_T7 (SEQ ID NO: 51) | 988 | 1072 |
| HUMPHOSLIP_PEA_2_T14 (SEQ ID NO: 52) | 981 | 1065 |
| HUMPHOSLIP_PEA_2_T16 (SEQ ID NO: 53) | 889 | 973 |
| HUMPHOSLIP_PEA_2_T17 (SEQ ID NO: 54) | 611 | 695 |
| HUMPHOSLIP_PEA_2_T18 (SEQ ID NO: 55) | 825 | 909 |
| HUMPHOSLIP_PEA_2_T19 (SEQ ID NO: 56) | 896 | 980 |

Segment cluster HUMPHOSLIP_PEA_{—}2_node_{—}33 (SEQ ID NO:389) according to the present invention is supported by 173 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 49 below describes the starting and ending position of this segment on each transcript.

TABLE 49
Segment location on transcripts

| | Segment | Segment |
|---|---|---|
| | starting | ending |
| Transcript name | position | position |
| HUMPHOSLIP_PEA_2_T6 (SEQ ID NO: 50) | 935 | 970 |
| HUMPHOSLIP_PEA_2_T7 (SEQ ID NO: 51) | 1073 | 1108 |
| HUMPHOSLIP_PEA_2_T14 (SEQ ID NO: 52) | 1066 | 1101 |
| HUMPHOSLIP_PEA_2_T16 (SEQ ID NO: 53) | 974 | 1009 |
| HUMPHOSLIP_PEA_2_T17 (SEQ ID NO: 54) | 696 | 731 |
| HUMPHOSLIP_PEA_2_T18 (SEQ ID NO: 55) | 910 | 945 |
| HUMPHOSLIP_PEA_2_T19 (SEQ ID NO: 56) | 981 | 1016 |

Segment cluster HUMPHOSLIP_PEA_{—}2_node_{—}36 (SEQ ID NO:390) according to the present invention is supported by 163 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 50 below describes the starting and ending position of this segment on each transcript.

TABLE 50
Segment location on transcripts

| | Segment | Segment |
|---|---|---|
| | starting | ending |
| Transcript name | position | position |
| HUMPHOSLIP_PEA_2_T6 (SEQ ID NO: 50) | 1112 | 1156 |
| HUMPHOSLIP_PEA_2_T7 (SEQ ID NO: 51) | 1250 | 1294 |
| HUMPHOSLIP_PEA_2_T14 (SEQ ID NO: 52) | 1243 | 1287 |
| HUMPHOSLIP_PEA_2_T16 (SEQ ID NO: 53) | 1151 | 1195 |
| HUMPHOSLIP_PEA_2_T17 (SEQ ID NO: 54) | 873 | 917 |
| HUMPHOSLIP_PEA_2_T18 (SEQ ID NO: 55) | 1087 | 1131 |
| HUMPHOSLIP_PEA_2_T19 (SEQ ID NO: 56) | 1158 | 1202 |

Segment cluster HUMPHOSLIP_PEA_{—}2_node_{—}37 (SEQ ID NO:391) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 51 below describes the starting and ending position of this segment on each transcript.

TABLE 51
Segment location on transcripts

| | Segment | Segment |
|---|---|---|
| | starting | ending |
| Transcript name | position | position |
| HUMPHOSLIP_PEA_2_T6 (SEQ ID NO: 50) | 1157 | 1171 |
| HUMPHOSLIP_PEA_2_T7 (SEQ ID NO: 51) | 1295 | 1309 |
| HUMPHOSLIP_PEA_2_T14 (SEQ ID NO: 52) | 1288 | 1302 |
| HUMPHOSLIP_PEA_2_T16 (SEQ ID NO: 53) | 1196 | 1210 |
| HUMPHOSLIP_PEA_2_T17 (SEQ ID NO: 54) | 918 | 932 |
| HUMPHOSLIP_PEA_2_T18 (SEQ ID NO: 55) | 1132 | 1146 |
| HUMPHOSLIP_PEA_2_T19 (SEQ ID NO: 56) | 1203 | 1217 |

Segment cluster HUMPHOSLIP_PEA_{—}2_node_{—}39 (SEQ ID NO:392) according to the present invention is supported by 166 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 52 below describes the starting and ending position of this segment on each transcript.

TABLE 52
Segment location on transcripts

| | Segment | Segment |
|---|---|---|
| | starting | ending |
| Transcript name | position | position |
| HUMPHOSLIP_PEA_2_T6 (SEQ ID NO: 50) | 1172 | 1201 |
| HUMPHOSLIP_PEA_2_T7 (SEQ ID NO: 51) | 1310 | 1339 |
| HUMPHOSLIP_PEA_2_T14 (SEQ ID NO: 52) | 1303 | 1332 |
| HUMPHOSLIP_PEA_2_T16 (SEQ ID NO: 53) | 1211 | 1240 |
| HUMPHOSLIP_PEA_2_T17 (SEQ ID NO: 54) | 933 | 962 |
| HUMPHOSLIP_PEA_2_T18 (SEQ ID NO: 55) | 1147 | 1176 |
| HUMPHOSLIP_PEA_2_T19 (SEQ ID NO: 56) | 1218 | 1247 |

Segment cluster HUMPHOSLIP_PEA_{—}2_node_{—}40 (SEQ ID NO:393) according to the present invention is supported by 199 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 53 below describes the starting and ending position of this segment on each transcript.

TABLE 53
Segment location on transcripts

| | Segment | Segment |
|---|---|---|
| | starting | ending |
| Transcript name | position | position |
| HUMPHOSLIP_PEA_2_T6 (SEQ ID NO: 50) | 1202 | 1288 |
| HUMPHOSLIP_PEA_2_T7 (SEQ ID NO: 51) | 1340 | 1426 |
| HUMPHOSLIP_PEA_2_T14 (SEQ ID NO: 52) | 1333 | 1419 |
| HUMPHOSLIP_PEA_2_T16 (SEQ ID NO: 53) | 1241 | 1327 |
| HUMPHOSLIP_PEA_2_T17 (SEQ ID NO: 54) | 963 | 1049 |
| HUMPHOSLIP_PEA_2_T18 (SEQ ID NO: 55) | 1177 | 1263 |
| HUMPHOSLIP_PEA_2_T19 (SEQ ID NO: 56) | 1248 | 1334 |

Segment cluster HUMPHOSLIP_PEA_{—}2_node_{—}41 (SEQ ID NO:394) according to the present invention is supported by 186 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 54 below describes the starting and ending position of this segment on each transcript.

TABLE 54
Segment location on transcripts

| | Segment | Segment |
|---|---|---|
| | starting | ending |
| Transcript name | position | position |
| HUMPHOSLIP_PEA_2_T6 (SEQ ID NO: 50) | 1289 | 1318 |
| HUMPHOSLIP_PEA_2_T7 (SEQ ID NO: 51) | 1427 | 1456 |
| HUMPHOSLIP_PEA_2_T14 (SEQ ID NO: 52) | 1420 | 1449 |
| HUMPHOSLIP_PEA_2_T16 (SEQ ID NO: 53) | 1328 | 1357 |
| HUMPHOSLIP_PEA_2_T17 (SEQ ID NO: 54) | 1050 | 1079 |
| HUMPHOSLIP_PEA_2_T18 (SEQ ID NO: 55) | 1264 | 1293 |
| HUMPHOSLIP_PEA_2_T19 (SEQ ID NO: 56) | 1335 | 1364 |

Segment cluster HUMPHOSLIP_PEA_{—}2_node_{—}42 (SEQ ID NO:395) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 55 below describes the starting and ending position of this segment on each transcript.

TABLE 55
Segment location on transcripts

| | Segment | Segment |
|---|---|---|
| | starting | ending |
| Transcript name | position | position |
| HUMPHOSLIP_PEA_2_T6 (SEQ ID NO: 50) | 1319 | 1336 |
| HUMPHOSLIP_PEA_2_T7 (SEQ ID NO: 51) | 1457 | 1474 |
| HUMPHOSLIP_PEA_2_T14 (SEQ ID NO: 52) | 1450 | 1467 |
| HUMPHOSLIP_PEA_2_T16 (SEQ ID NO: 53) | 1358 | 1375 |
| HUMPHOSLIP_PEA_2_T17 (SEQ ID NO: 54) | 1080 | 1097 |
| HUMPHOSLIP_PEA_2_T18 (SEQ ID NO: 55) | 1294 | 1311 |
| HUMPHOSLIP_PEA_2_T19 (SEQ ID NO: 56) | 1365 | 1382 |

Segment cluster HUMPHOSLIP_PEA_{—}2_node_{—}44 (SEQ ID NO:396) according to the present invention is supported by 185 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 56 below describes the starting and ending position of this segment on each transcript.

TABLE 56
Segment location on transcripts

| | Segment | Segment |
|---|---|---|
| | starting | ending |
| Transcript name | position | position |
| HUMPHOSLIP_PEA_2_T6 (SEQ ID NO: 50) | 1337 | 1363 |
| HUMPHOSLIP_PEA_2_T7 (SEQ ID NO: 51) | 1475 | 1501 |
| HUMPHOSLIP_PEA_2_T14 (SEQ ID NO: 52) | 1468 | 1494 |
| HUMPHOSLIP_PEA_2_T16 (SEQ ID NO: 53) | 1376 | 1402 |
| HUMPHOSLIP_PEA_2_T17 (SEQ ID NO: 54) | 1098 | 1124 |
| HUMPHOSLIP_PEA_2_T18 (SEQ ID NO: 55) | 1312 | 1338 |
| HUMPHOSLIP_PEA_2_T19 (SEQ ID NO: 56) | 1383 | 1409 |

Segment cluster HUMPHOSLIP_PEA_{—}2_node_{—}45 (SEQ ID NO:397) according to the present invention is supported by 197 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 57 below describes the starting and ending position of this segment on each transcript.

TABLE 57
Segment location on transcripts

| | Segment | Segment |
|---|---|---|
| | starting | ending |
| Transcript name | position | position |
| HUMPHOSLIP_PEA_2_T6 (SEQ ID NO: 50) | 1364 | 1404 |
| HUMPHOSLIP_PEA_2_T7 (SEQ ID NO: 51) | 1502 | 1542 |
| HUMPHOSLIP_PEA_2_T14 (SEQ ID NO: 52) | 1495 | 1535 |
| HUMPHOSLIP_PEA_2_T16 (SEQ ID NO: 53) | 1403 | 1443 |
| HUMPHOSLIP_PEA_2_T17 (SEQ ID NO: 54) | 1125 | 1165 |
| HUMPHOSLIP_PEA_2_T18 (SEQ ID NO: 55) | 1339 | 1379 |
| HUMPHOSLIP_PEA_2_T19 (SEQ ID NO: 56) | 1410 | 1450 |

Segment cluster HUMPHOSLIP_PEA_{—}2_node_{—}47 (SEQ ID NO:398) according to the present invention is supported by 223 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 58 below describes the starting and ending position of this segment on each transcript.

TABLE 58
Segment location on transcripts

| | Segment | Segment |
|---|---|---|
| | starting | ending |
| Transcript name | position | position |
| HUMPHOSLIP_PEA_2_T6 (SEQ ID NO: 50) | 1405 | 1447 |
| HUMPHOSLIP_PEA_2_T7 (SEQ ID NO: 51) | 1543 | 1585 |
| HUMPHOSLIP_PEA_2_T14 (SEQ ID NO: 52) | 1536 | 1578 |
| HUMPHOSLIP_PEA_2_T16 (SEQ ID NO: 53) | 1444 | 1486 |
| HUMPHOSLIP_PEA_2_T17 (SEQ ID NO: 54) | 1166 | 1208 |
| HUMPHOSLIP_PEA_2_T18 (SEQ ID NO: 55) | 1380 | 1422 |
| HUMPHOSLIP_PEA_2_T19 (SEQ ID NO: 56) | 1451 | 1493 |

Segment cluster HUMPHOSLIP_PEA_{—}2_node_{—}51 (SEQ ID NO:399) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 59 below describes the starting and ending position of this segment on each transcript.

TABLE 59
Segment location on transcripts

| | Segment | Segment |
|---|---|---|
| | starting | ending |
| Transcript name | position | position |
| HUMPHOSLIP_PEA_2_T6 (SEQ ID NO: 50) | 1448 | 1462 |
| HUMPHOSLIP_PEA_2_T7 (SEQ ID NO: 51) | 1586 | 1600 |
| HUMPHOSLIP_PEA_2_T14 (SEQ ID NO: 52) | 1579 | 1593 |
| HUMPHOSLIP_PEA_2_T16 (SEQ ID NO: 53) | 1487 | 1501 |
| HUMPHOSLIP_PEA_2_T17 (SEQ ID NO: 54) | 1209 | 1223 |
| HUMPHOSLIP_PEA_2_T18 (SEQ ID NO: 55) | 1423 | 1437 |
| HUMPHOSLIP_PEA_2_T19 (SEQ ID NO: 56) | 1494 | 1508 |

Segment cluster HUMPHOSLIP_PEA_{—}2_node_{—}52 (SEQ ID NO:400) according to the present invention is supported by 235 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 60 below describes the starting and ending position of this segment on each transcript.

TABLE 60
Segment location on transcripts

| | Segment | Segment |
|---|---|---|
| | starting | ending |
| Transcript name | position | position |
| HUMPHOSLIP_PEA_2_T6 (SEQ ID NO: 50) | 1463 | 1511 |
| HUMPHOSLIP_PEA_2_T7 (SEQ ID NO: 51) | 1601 | 1649 |
| HUMPHOSLIP_PEA_2_T14 (SEQ ID NO: 52) | 1594 | 1642 |
| HUMPHOSLIP_PEA_2_T16 (SEQ ID NO: 53) | 1502 | 1550 |
| HUMPHOSLIP_PEA_2_T17 (SEQ ID NO: 54) | 1224 | 1272 |
| HUMPHOSLIP_PEA_2_T18 (SEQ ID NO: 55) | 1438 | 1486 |
| HUMPHOSLIP_PEA_2_T19 (SEQ ID NO: 56) | 1509 | 1557 |

Segment cluster HUMPHOSLIP_PEA_{—}2_node_{—}53 (SEQ ID NO:401) according to the present invention is supported by 5 libraries. The number of libraries was determined as previously described. This segment can be found in the following transcript(s): HUMPHOSLIP_PEA_{—}2_T19 (SEQ ID NO:56). Table 61 below describes the starting and ending position of this segment on each transcript.

TABLE 61
Segment location on transcripts

| | Segment | Segment |
|---|---|---|
| | starting | ending |
| Transcript name | position | position |
| HUMPHOSLIP_PEA_2_T19 (SEQ ID NO: 56) | 1558 | 1640 |

Segment cluster HUMPHOSLIP_PEA_{—}2_node_{—}54 (SEQ ID NO:402) according to the present invention is supported by 236 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 62 below describes the starting and ending position of this segment on each transcript.

TABLE 62
Segment location on transcripts

| | Segment | Segment |
|---|---|---|
| | starting | ending |
| Transcript name | position | position |
| HUMPHOSLIP_PEA_2_T6 (SEQ ID NO: 50) | 1512 | 1552 |
| HUMPHOSLIP_PEA_2_T7 (SEQ ID NO: 51) | 1650 | 1690 |
| HUMPHOSLIP_PEA_2_T14 (SEQ ID NO: 52) | 1643 | 1683 |
| HUMPHOSLIP_PEA_2_T16 (SEQ ID NO: 53) | 1551 | 1591 |
| HUMPHOSLIP_PEA_2_T17 (SEQ ID NO: 54) | 1273 | 1313 |
| HUMPHOSLIP_PEA_2_T18 (SEQ ID NO: 55) | 1487 | 1527 |
| HUMPHOSLIP_PEA_2_T19 (SEQ ID NO: 56) | 1641 | 1681 |

Segment cluster HUMPHOSLIP_PEA_{—}2_node_{—}55 (SEQ ID NO:403) according to the present invention is supported by 232 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 63 below describes the starting and ending position of this segment on each transcript.

TABLE 63
Segment location on transcripts

| | Segment | Segment |
|---|---|---|
| | starting | ending |
| Transcript name | position | position |
| HUMPHOSLIP_PEA_2_T6 (SEQ ID NO: 50) | 1553 | 1588 |
| HUMPHOSLIP_PEA_2_T7 (SEQ ID NO: 51) | 1691 | 1726 |
| HUMPHOSLIP_PEA_2_T14 (SEQ ID NO: 52) | 1684 | 1719 |
| HUMPHOSLIP_PEA_2_T16 (SEQ ID NO: 53) | 1592 | 1627 |
| HUMPHOSLIP_PEA_2_T17 (SEQ ID NO: 54) | 1314 | 1349 |
| HUMPHOSLIP_PEA_2_T18 (SEQ ID NO: 55) | 1528 | 1563 |
| HUMPHOSLIP_PEA_2_T19 (SEQ ID NO: 56) | 1682 | 1717 |

Segment cluster HUMPHOSLIP_PEA_{—}2_node_{—}58 (SEQ ID NO:404) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 64 below describes the starting and ending position of this segment on each transcript.

TABLE 64
Segment location on transcripts

| | Segment | Segment |
|---|---|---|
| | starting | ending |
| Transcript name | position | position |
| HUMPHOSLIP_PEA_2_T6 (SEQ ID NO: 50) | 1589 | 1612 |
| HUMPHOSLIP_PEA_2_T7 (SEQ ID NO: 51) | 1727 | 1750 |
| HUMPHOSLIP_PEA_2_T14 (SEQ ID NO: 52) | 1720 | 1743 |
| HUMPHOSLIP_PEA_2_T16 (SEQ ID NO: 53) | 1628 | 1651 |
| HUMPHOSLIP_PEA_2_T17 (SEQ ID NO: 54) | 1350 | 1373 |
| HUMPHOSLIP_PEA_2_T18 (SEQ ID NO: 55) | 1564 | 1587 |
| HUMPHOSLIP_PEA_2_T19 (SEQ ID NO: 56) | 1718 | 1741 |

Segment cluster HUMPHOSLIP_PEA_{—}2_node_{—}59 (SEQ ID NO:405) according to the present invention is supported by 230 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 65 below describes the starting and ending position of this segment on each transcript.

TABLE 65
Segment location on transcripts

| | Segment | Segment |
|---|---|---|
| | starting | ending |
| Transcript name | position | position |
| HUMPHOSLIP_PEA_2_T6 (SEQ ID NO: 50) | 1613 | 1648 |
| HUMPHOSLIP_PEA_2_T7 (SEQ ID NO: 51) | 1751 | 1786 |
| HUMPHOSLIP_PEA_2_T14 (SEQ ID NO: 52) | 1744 | 1779 |
| HUMPHOSLIP_PEA_2_T16 (SEQ ID NO: 53) | 1652 | 1687 |
| HUMPHOSLIP_PEA_2_T17 (SEQ ID NO: 54) | 1374 | 1409 |
| HUMPHOSLIP_PEA_2_T18 (SEQ ID NO: 55) | 1588 | 1623 |
| HUMPHOSLIP_PEA_2_T19 (SEQ ID NO: 56) | 1742 | 1777 |

Segment cluster HUMPHOSLIP_PEA_{—}2_node_{—}60 (SEQ ID NO:406) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 66 below describes the starting and ending position of this segment on each transcript.

TABLE 66
Segment location on transcripts

| | Segment | Segment |
|---|---|---|
| | starting | ending |
| Transcript name | position | position |
| HUMPHOSLIP_PEA_2_T6 (SEQ ID NO: 50) | 1649 | 1671 |
| HUMPHOSLIP_PEA_2_T7 (SEQ ID NO: 51) | 1787 | 1809 |
| HUMPHOSLIP_PEA_2_T14 (SEQ ID NO: 52) | 1780 | 1802 |
| HUMPHOSLIP_PEA_2_T16 (SEQ ID NO: 53) | 1688 | 1710 |
| HUMPHOSLIP_PEA_2_T17 (SEQ ID NO: 54) | 1410 | 1432 |
| HUMPHOSLIP_PEA_2_T18 (SEQ ID NO: 55) | 1624 | 1646 |
| HUMPHOSLIP_PEA_2_T19 (SEQ ID NO: 56) | 1778 | 1800 |

Segment cluster HUMPHOSLIP_PEA_{—}2_node_{—}61 (SEQ ID NO:407) according to the present invention can be found in the following transcript(s): HUMPHOSLIP_PEA_{—}2_T6 (SEQ ID NO:50) (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54) (SEQ ID NO:55) and HUMPHOSLIP_PEA_{—}2_T19 (SEQ ID NO:56). Table 67 below describes the starting and ending position of this segment on each transcript.

TABLE 67
Segment location on transcripts

| | Segment | Segment |
|---|---|---|
| | starting | ending |
| Transcript name | position | position |
| HUMPHOSLIP_PEA_2_T6 (SEQ ID NO: 50) | 1672 | 1680 |
| HUMPHOSLIP_PEA_2_T7 (SEQ ID NO: 51) | 1810 | 1818 |
| HUMPHOSLIP_PEA_2_T14 (SEQ ID NO: 52) | 1803 | 1811 |
| HUMPHOSLIP_PEA_2_T16 (SEQ ID NO: 53) | 1711 | 1719 |
| HUMPHOSLIP_PEA_2_T17 (SEQ ID NO: 54) | 1433 | 1441 |
| HUMPHOSLIP_PEA_2_T18 (SEQ ID NO: 55) | 1647 | 1655 |
| HUMPHOSLIP_PEA_2_T19 (SEQ ID NO: 56) | 1801 | 1809 |

Segment cluster HUMPHOSLIP_PEA_{—}2_node_{—}62 (SEQ ID NO:408) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 68 below describes the starting and ending position of this segment on each transcript.

TABLE 68
Segment location on transcripts

| | Segment | Segment |
|---|---|---|
| | starting | ending |
| Transcript name | position | position |
| HUMPHOSLIP_PEA_2_T6 (SEQ ID NO: 50) | 1681 | 1703 |
| HUMPHOSLIP_PEA_2_T7 (SEQ ID NO: 51) | 1819 | 1841 |
| HUMPHOSLIP_PEA_2_T14 (SEQ ID NO: 52) | 1812 | 1834 |
| HUMPHOSLIP_PEA_2_T16 (SEQ ID NO: 53) | 1720 | 1742 |
| HUMPHOSLIP_PEA_2_T17 (SEQ ID NO: 54) | 1442 | 1464 |
| HUMPHOSLIP_PEA_2_T18 (SEQ ID NO: 55) | 1656 | 1678 |
| HUMPHOSLIP_PEA_2_T19 (SEQ ID NO: 56) | 1810 | 1832 |

Segment cluster HUMPHOSLIP_PEA_{—}2_node_{—}63 (SEQ ID NO:409) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 69 below describes the starting and ending position of this segment on each transcript.

TABLE 69
Segment location on transcripts

| | Segment | Segment |
|---|---|---|
| | starting | ending |
| Transcript name | position | position |
| HUMPHOSLIP_PEA_2_T6 (SEQ ID NO: 50) | 1704 | 1727 |
| HUMPHOSLIP_PEA_2_T7 (SEQ ID NO: 51) | 1842 | 1865 |
| HUMPHOSLIP_PEA_2_T14 (SEQ ID NO: 52) | 1835 | 1858 |
| HUMPHOSLIP_PEA_2_T16 (SEQ ID NO: 53) | 1743 | 1766 |
| HUMPHOSLIP_PEA_2_T17 (SEQ ID NO: 54) | 1465 | 1488 |
| HUMPHOSLIP_PEA_2_T18 (SEQ ID NO: 55) | 1679 | 1702 |
| HUMPHOSLIP_PEA_2_T19 (SEQ ID NO: 56) | 1833 | 1856 |

Segment cluster HUMPHOSLIP_PEA_{—}2_node_{—}64 (SEQ ID NO:410) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 70 below describes the starting and ending position of this segment on each transcript.

TABLE 70
Segment location on transcripts

| | Segment | Segment |
|---|---|---|
| | starting | ending |
| Transcript name | position | position |
| HUMPHOSLIP_PEA_2_T6 (SEQ ID NO: 50) | 1728 | 1734 |
| HUMPHOSLIP_PEA_2_T7 (SEQ ID NO: 51) | 1866 | 1872 |
| HUMPHOSLIP_PEA_2_T14 (SEQ ID NO: 52) | 1859 | 1865 |
| HUMPHOSLIP_PEA_2_T16 (SEQ ID NO: 53) | 1767 | 1773 |
| HUMPHOSLIP_PEA_2_T17 (SEQ ID NO: 54) | 1489 | 1495 |
| HUMPHOSLIP_PEA_2_T18 (SEQ ID NO: 55) | 1703 | 1709 |
| HUMPHOSLIP_PEA_2_T19 (SEQ ID NO: 56) | 1857 | 1863 |

Segment cluster HUMPHOSLIP_PEA_{—}2_node_{—}65 (SEQ ID NO:411) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 71 below describes the starting and ending position of this segment on each transcript.

TABLE 71
Segment location on transcripts

| | Segment | Segment |
|---|---|---|
| | starting | ending |
| Transcript name | position | position |
| HUMPHOSLIP_PEA_2_T6 (SEQ ID NO: 50) | 1735 | 1754 |
| HUMPHOSLIP_PEA_2_T7 (SEQ ID NO: 51) | 1873 | 1892 |
| HUMPHOSLIP_PEA_2_T14 (SEQ ID NO: 52) | 1866 | 1885 |
| HUMPHOSLIP_PEA_2_T16 (SEQ ID NO: 53) | 1774 | 1793 |
| HUMPHOSLIP_PEA_2_T17 (SEQ ID NO: 54) | 1496 | 1515 |
| HUMPHOSLIP_PEA_2_T18 (SEQ ID NO: 55) | 1710 | 1729 |
| HUMPHOSLIP_PEA_2_T19 (SEQ ID NO: 56) | 1864 | 1883 |

Segment cluster HUMPHOSLIP_PEA_{—}2_node_{—}66 (SEQ ID NO:412) according to the present invention is supported by 180 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 72 below describes the starting and ending position of this segment on each transcript.

TABLE 72
Segment location on transcripts

| | Segment | Segment |
|---|---|---|
| | starting | ending |
| Transcript name | position | position |
| HUMPHOSLIP_PEA_2_T6 (SEQ ID NO: 50) | 1755 | 1844 |
| HUMPHOSLIP_PEA_2_T7 (SEQ ID NO: 51) | 1893 | 1982 |
| HUMPHOSLIP_PEA_2_T14 (SEQ ID NO: 52) | 1886 | 1975 |
| HUMPHOSLIP_PEA_2_T16 (SEQ ID NO: 53) | 1794 | 1883 |
| HUMPHOSLIP_PEA_2_T17 (SEQ ID NO: 54) | 1516 | 1605 |
| HUMPHOSLIP_PEA_2_T18 (SEQ ID NO: 55) | 1730 | 1819 |
| HUMPHOSLIP_PEA_2_T19 (SEQ ID NO: 56) | 1884 | 1973 |

Segment cluster HUMPHOSLIP_PEA_{—}2_node_{—}67 (SEQ ID NO:413) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 73 below describes the starting and ending position of this segment on each transcript.

TABLE 73
Segment location on transcripts

| | Segment | Segment |
|---|---|---|
| | starting | ending |
| Transcript name | position | position |
| HUMPHOSLIP_PEA_2_T6 (SEQ ID NO: 50) | 1845 | 1866 |
| HUMPHOSLIP_PEA_2_T7 (SEQ ID NO: 51) | 1983 | 2004 |
| HUMPHOSLIP_PEA_2_T14 (SEQ ID NO: 52) | 1976 | 1997 |
| HUMPHOSLIP_PEA_2_T16 (SEQ ID NO: 53) | 1884 | 1905 |
| HUMPHOSLIP_PEA_2_T17 (SEQ ID NO: 54) | 1606 | 1627 |
| HUMPHOSLIP_PEA_2_T18 (SEQ ID NO: 55) | 1820 | 1841 |
| HUMPHOSLIP_PEA_2_T19 (SEQ ID NO: 56) | 1974 | 1995 |

Segment cluster HUMPHOSLIP_PEA_{—}2_node_{—}69 (SEQ ID NO:414) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 74 below describes the starting and ending position of this segment on each transcript.

TABLE 74
Segment location on transcripts

| | Segment | Segment |
|---|---|---|
| | starting | ending |
| Transcript name | position | position |
| HUMPHOSLIP_PEA_2_T6 (SEQ ID NO: 50) | 2286 | 2297 |
| HUMPHOSLIP_PEA_2_T7 (SEQ ID NO: 51) | 2424 | 2435 |
| HUMPHOSLIP_PEA_2_T14 (SEQ ID NO: 52) | 2417 | 2428 |
| HUMPHOSLIP_PEA_2_T16 (SEQ ID NO: 53) | 2325 | 2336 |
| HUMPHOSLIP_PEA_2_T17 (SEQ ID NO: 54) | 2047 | 2058 |
| HUMPHOSLIP_PEA_2_T18 (SEQ ID NO: 55) | 2261 | 2272 |
| HUMPHOSLIP_PEA_2_T19 (SEQ ID NO: 56) | 2415 | 2426 |

Segment cluster HUMPHOSLIP_PEA_{—}2_node_{—}71 (SEQ ID NO:415) according to the present invention can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 75 below describes the starting and ending position of this segment on each transcript.

TABLE 75
Segment location on transcripts

| | Segment | Segment |
|---|---|---|
| | starting | ending |
| Transcript name | position | position |
| HUMPHOSLIP_PEA_2_T6 (SEQ ID NO: 50) | 2530 | 2542 |
| HUMPHOSLIP_PEA_2_T7 (SEQ ID NO: 51) | 2668 | 2680 |
| HUMPHOSLIP_PEA_2_T14 (SEQ ID NO: 52) | 2661 | 2673 |
| HUMPHOSLIP_PEA_2_T16 (SEQ ID NO: 53) | 2569 | 2581 |
| HUMPHOSLIP_PEA_2_T17 (SEQ ID NO: 54) | 2291 | 2303 |
| HUMPHOSLIP_PEA_2_T18 (SEQ ID NO: 55) | 2505 | 2517 |
| HUMPHOSLIP_PEA_2_T19 (SEQ ID NO: 56) | 2659 | 2671 |

Segment cluster HUMPHOSLIP_PEA_{—}2_node_{—}72 (SEQ ID NO:416) according to the present invention is supported by 7 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 76 below describes the starting and ending position of this segment on each transcript.

TABLE 76
Segment location on transcripts

| | Segment | Segment |
|---|---|---|
| | starting | ending |
| Transcript name | position | position |
| HUMPHOSLIP_PEA_2_T6 (SEQ ID NO: 50) | 2543 | 2647 |
| HUMPHOSLIP_PEA_2_T7 (SEQ ID NO: 51) | 2681 | 2785 |
| HUMPHOSLIP_PEA_2_T14 (SEQ ID NO: 52) | 2674 | 2778 |
| HUMPHOSLIP_PEA_2_T16 (SEQ ID NO: 53) | 2582 | 2686 |
| HUMPHOSLIP_PEA_2_T17 (SEQ ID NO: 54) | 2304 | 2408 |
| HUMPHOSLIP_PEA_2_T18 (SEQ ID NO: 55) | 2518 | 2622 |
| HUMPHOSLIP_PEA_2_T19 (SEQ ID NO: 56) | 2672 | 2776 |

Segment cluster HUMPHOSLIP_PEA_{—}2_node_{—}73 (SEQ ID NO:417) according to the present invention is supported by 5 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 77 below describes the starting and ending position of this segment on each transcript.

TABLE 77
Segment location on transcripts

| | Segment | Segment |
|---|---|---|
| | starting | ending |
| Transcript name | position | position |
| HUMPHOSLIP_PEA_2_T6 (SEQ ID NO: 50) | 2648 | 2755 |
| HUMPHOSLIP_PEA_2_T7 (SEQ ID NO: 51) | 2786 | 2893 |
| HUMPHOSLIP_PEA_2_T14 (SEQ ID NO: 52) | 2779 | 2886 |
| HUMPHOSLIP_PEA_2_T16 (SEQ ID NO: 53) | 2687 | 2794 |
| HUMPHOSLIP_PEA_2_T17 (SEQ ID NO: 54) | 2409 | 2516 |
| HUMPHOSLIP_PEA_2_T18 (SEQ ID NO: 55) | 2623 | 2730 |
| HUMPHOSLIP_PEA_2_T19 (SEQ ID NO: 56) | 2777 | 2884 |

Segment cluster HUMPHOSLIP_PEA_{—}2_node_{—}74 (SEQ ID NO:418) according to the present invention is supported by 10 libraries. The number of libraries was determined as previously described. This segment can be found in the following transcript(s): HUMPHOSLIP_PEA_{—}2_T6 (SEQ ID NO:50), HUMPHOSLIP_PEA_{—}2_T7 (SEQ ID NO:51), HUMPHOSLIP_PEA_{—}2_T14 (SEQ ID NO:52), HUMPHOSLIP_PEA_{—}2_T16 (SEQ ID NO:53), HUMPHOSLIP_PEA_{—}2_T17 (SEQ ID NO:54), HUMPHOSLIP_PEA_{—}2_T18 (SEQ ID NO:55) and HUMPHOSLIP_PEA_{—}2_T19 (SEQ ID NO:56). Table 78 below describes the starting and ending position of this segment on each transcript.

TABLE 78
Segment location on transcripts

| | Segment | Segment |
|---|---|---|
| | starting | ending |
| Transcript name | position | position |
| HUMPHOSLIP_PEA_2_T6 (SEQ ID NO: 50) | 2756 | 2845 |
| HUMPHOSLIP_PEA_2_T7 (SEQ ID NO: 51) | 2894 | 2983 |
| HUMPHOSLIP_PEA_2_T14 (SEQ ID NO: 52) | 2887 | 2976 |
| HUMPHOSLIP_PEA_2_T16 (SEQ ID NO: 53) | 2795 | 2884 |
| HUMPHOSLIP_PEA_2_T17 (SEQ ID NO: 54) | 2517 | 2606 |
| HUMPHOSLIP_PEA_2_T18 (SEQ ID NO: 55) | 2731 | 2820 |
| HUMPHOSLIP_PEA_2_T19 (SEQ ID NO: 56) | 2885 | 2974 |

### Variant Protein Alignment to the Previously Known Protein:

Sequence name: PLTP_HUMAN (SEQ ID NO: 636)
Sequence documentation:
Alignment of:
HUMPHOSLIP_PEA_2_P10 (SEQ ID NO: 581) × PLTP_HUMAN (SEQ ID
NO: 636) . .
Alignment segment 1/1:

| | | | |
|---|---|---|---|
| Quality: | 3716.00 | Escore: | 0 |
| Matching length: | 398 | Total length: | 493 |
| Matching Percent Similarity: | 100.00 | Matching Percent Identity: | 100.00 |
| Total Percent Similarity: | 80.73 | Total Percent Identity: | 80.73 |
| Gaps: | 1 | | |

Alignment:

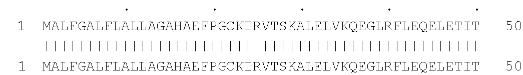

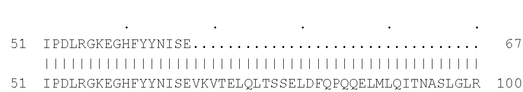

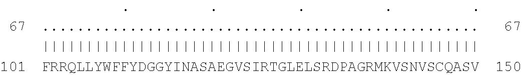

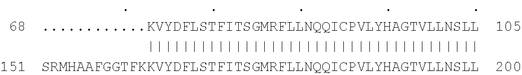

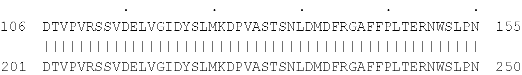

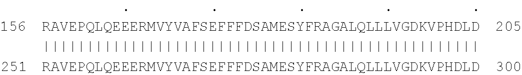

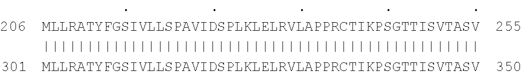

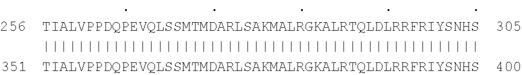

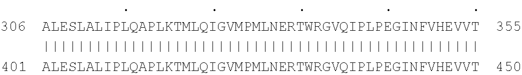

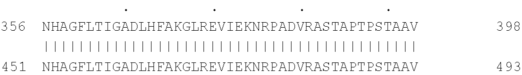

Sequence name: PLTP_HUMAN (SEQ ID NO: 636)
Sequence documentation:
Alignment of:
HUMPHOSLIP_PEA_2_P12 (SEQ ID NO: 582) × PLTP_HUMAN (SEQ ID
NO: 636) . .
Alignment segment 1/1:

| | | | |
|---|---|---|---|
| Quality: | 4101.00 | Escore: | 0 |
| Matching length: | 427 | Total length: | 427 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

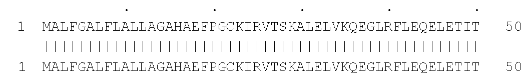

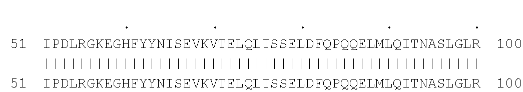

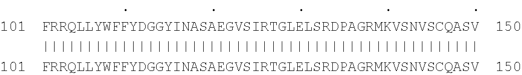

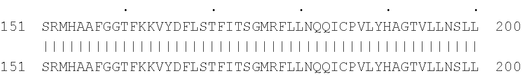

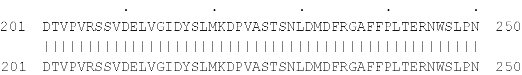

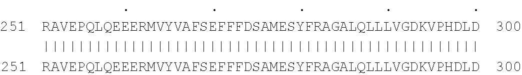

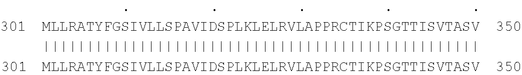

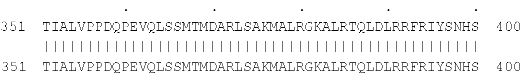

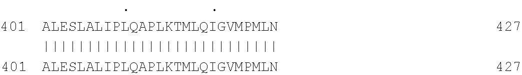

Sequence name: PLTP HUMAN (SEQ ID NO: 636)
Sequence documentation:
Alignment of:
HUMPHOSLIP_PEA_2_P31 (SEQ ID NO: 584) × PLTP_HUMAN (SEQ ID
NO: 636) . .
Alignment segment 1/1:

| | | | |
|---|---|---|---|
| Quality: | 639.00 | Escore: | 0 |
| Matching length: | 67 | Total length: | 67 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

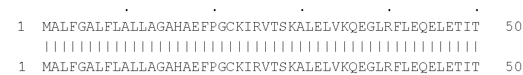

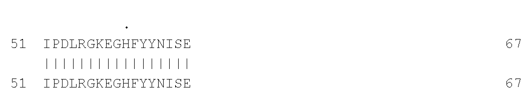

Sequence name: PLTP_HUMAN (SEQ ID NO: 636)
Sequence documentation:
Alignment of:
HUMPHOSLIP_PEA_2_P33 (SEQ ID NO: 585) × PLTP_HUMAN (SEQ ID
NO: 636) . .
Alignment segment 1/1:

| | | | |
|---|---|---|---|
| Quality: | 1767.00 | Escore: | 0 |
| Matching length: | 184 | Total length: | 184 |
| Matching Percent Similarity: | 100.00 | Matching Percent Identity: | 99.46 |
| Total Percent Similarity: | 100.00 | Total Percent Identity: | 99.46 |
| Gaps: | 0 | | |

Alignment:

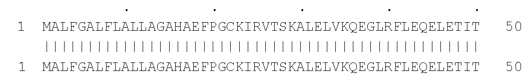

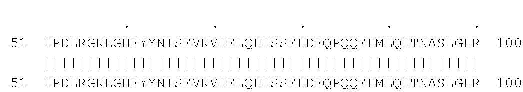

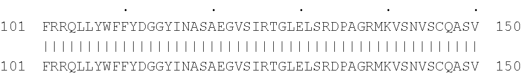

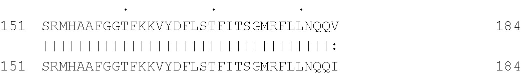

Sequence name: PLTP_HUMAN (SEQ ID NO: 636)
Sequence documentation:
Alignment of:
HUMPHOSLIP_PEA_2_P34 (SEQ ID NO: 586) × PLTP_HUMAN (SEQ ID
NO: 636) . .
Alignment segment 1/1:

| | | | |
|---|---|---|---|
| Quality: | 1971.00 | Escore: | 0 |
| Matching length: | 205 | Total length: | 205 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

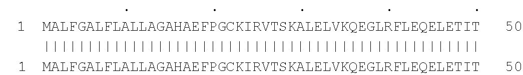

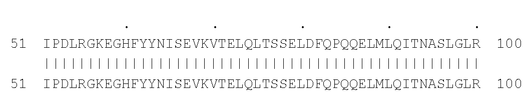

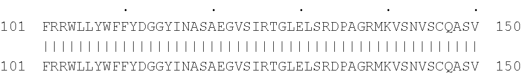

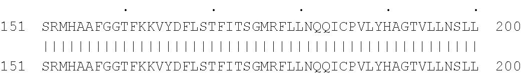

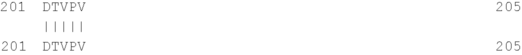

Sequence name: PLTP_HUMAN (SEQ ID NO: 636)
Sequence documentation:
Alignment of:
HUMPHOSLIP_PEA_2_P35 (SEQ ID NO: 587) × PLTP HUMAN (SEQ ID
NO: 636) . .
Alignment segment 1/1:

| | | | |
|---|---|---|---|
| Quality: | 1158.00 | Escore: | 0 |
| Matching length: | 132 | Total length: | 184 |
| Matching Percent Similarity: | 100.00 | Matching Percent Identity: | 98.48 |
| Total Percent Similarity: | 71.74 | Total Percent Identity: | 70.65 |
| Gaps: | 1 | | |

Alignment:

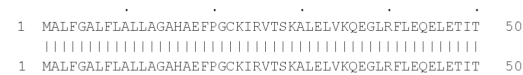

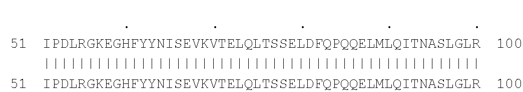

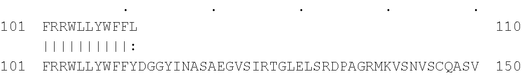

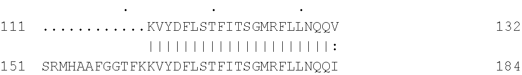

### Description for Cluster D11853

Cluster D11853 features 18 transcript(s) and 31 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| D11853_PEA_1_T1 | 57 |
| D11853_PEA_1_T3 | 58 |
| D11853_PEA_1_T7 | 59 |
| D11853_PEA_1_T8 | 60 |
| D11853_PEA_1_T9 | 61 |
| D11853_PEA_1_T10 | 62 |
| D11853_PEA_1_T13 | 63 |
| D11853_PEA_1_T14 | 64 |
| D11853_PEA_1_T15 | 65 |
| D11853_PEA_1_T16 | 66 |
| D11853_PEA_1_T17 | 67 |
| D11853_PEA_1_T19 | 68 |
| D11853_PEA_1_T21 | 69 |
| D11853_PEA_1_T23 | 70 |
| D11853_PEA_1_T24 | 71 |
| D11853_PEA_1_T25 | 72 |
| D11853_PEA_1_T26 | 73 |
| D11853_PEA_1_T27 | 74 |

TABLE 2
Segments of interest

| Segment Name | SEQ ID NO: |
|---|---|
| D11853_PEA_1_node_3 | 419 |
| D11853_PEA_1_node_6 | 420 |
| D11853_PEA_1_node_9 | 421 |
| D11853_PEA_1_node_17 | 422 |
| D11853_PEA_1_node_21 | 423 |
| D11853_PEA_1_node_22 | 424 |
| D11853_PEA_1_node_23 | 425 |
| D11853_PEA_1_node_25 | 426 |
| D11853_PEA_1_node_26 | 427 |
| D11853_PEA_1_node_27 | 428 |
| D11853_PEA_1_node_30 | 429 |
| D11853_PEA_1_node_32 | 430 |
| D11853_PEA_1_node_0 | 431 |
| D11853_PEA_1_node_1 | 432 |
| D11853_PEA_1_node_2 | 433 |
| D11853_PEA_1_node_4 | 434 |
| D11853_PEA_1_node_5 | 435 |
| D11853_PEA_1_node_7 | 436 |
| D11853_PEA_1_node_8 | 437 |
| D11853_PEA_1_node_10 | 438 |
| D11853_PEA_1_node_12 | 439 |
| D11853_PEA_1_node_13 | 440 |
| D11853_PEA_1_node_14 | 441 |
| D11853_PEA_1_node_15 | 442 |
| D11853_PEA_1_node_16 | 443 |
| D11853_PEA_1_node_18 | 444 |
| D11853_PEA_1_node_19 | 445 |
| D11853_PEA_1_node_20 | 446 |
| D11853_PEA_1_node_24 | 447 |
| D11853_PEA_1_node_28 | 448 |
| D11853_PEA_1_node_29 | 449 |

TABLE 3
Proteins of interest

| | SEQ ID | |
|---|---|---|
| Protein Name | NO: | Corresponding Transcript(s) |
| D11853_PEA_1_P1 | 588 | D11853_PEA_1_T1 (SEQ ID |
| | | NO: 57) |
| D11853_PEA_1_P2 | 589 | D11853_PEA_1_T3 (SEQ ID |
| | | NO: 58) |
| D11853_PEA_1_P7 | 590 | D11853_PEA_1_T10 (SEQ ID |
| | | NO: 62) |
| D11853_PEA_1_P9 | 591 | D11853_PEA_1_T13 (SEQ ID |
| | | NO: 63) |
| D11853_PEA_1_P10 | 592 | D11853_PEA_1_T14 (SEQ ID |
| | | NO: 64) |
| D11853_PEA_1_P11 | 593 | D11853_PEA_1_T15 (SEQ ID |
| | | NO: 65) |
| D11853_PEA_1_P12 | 594 | D11853_PEA_1_T16 (SEQ ID |
| | | NO: 66); D11853_PEA_1_T23 |
| | | (SEQ ID NO: 70) |
| D11853_PEA_1_P14 | 595 | D11853_PEA_1_T19 (SEQ ID |
| | | NO: 68) |
| D11853_PEA_1_P16 | 596 | D11853_PEA_1_T24 (SEQ ID |
| | | NO: 71) |
| D11853_PEA_1_P18 | 597 | D11853_PEA_1_T26 (SEQ ID |
| | | NO: 73) |
| D11853_PEA_1_P19 | 598 | D11853_PEA_1_T27 (SEQ ID |
| | | NO: 74) |
| D11853_PEA_1_P20 | 599 | D11853_PEA_1_T7 (SEQ ID |
| | | NO: 59); D11853_PEA_1_T17 |
| | | (SEQ ID NO: 67); |
| | | D11853_PEA_1_T25 (SEQ ID |
| | | NO: 72) |
| D11853_PEA_1_P21 | 600 | D11853_PEA_1_T8 (SEQ ID |
| | | NO: 60) |
| D11853_PEA_1_P22 | 601 | D11853_PEA_1_T9 (SEQ ID |
| | | NO: 61) |
| D11853_PEA_1_P24 | 602 | D11853_PEA_1_T21 (SEQ ID |
| | | NO: 69) |

These sequences are variants of the known protein Membrane associated protein SLP-2 (SwissProt accession identifier Q9UJZ1; known also according to the synonyms Stomatin-like protein 2; Stomatin-like 2; Hypothetical protein FLJ14499), SEQ ID NO: 637, referred to herein as the previously known protein.

The sequence for protein Membrane associated protein SLP-2 (SEQ ID NO:637) is given at the end of the application, as “Membrane associated protein SLP-2 amino acid sequence”.

The following GO Annotation(s) apply to the previously known protein. The following annotation(s) were found: ligand, which are annotation(s) related to Molecular Function; and cytoskeleton; membrane, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster D11853 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

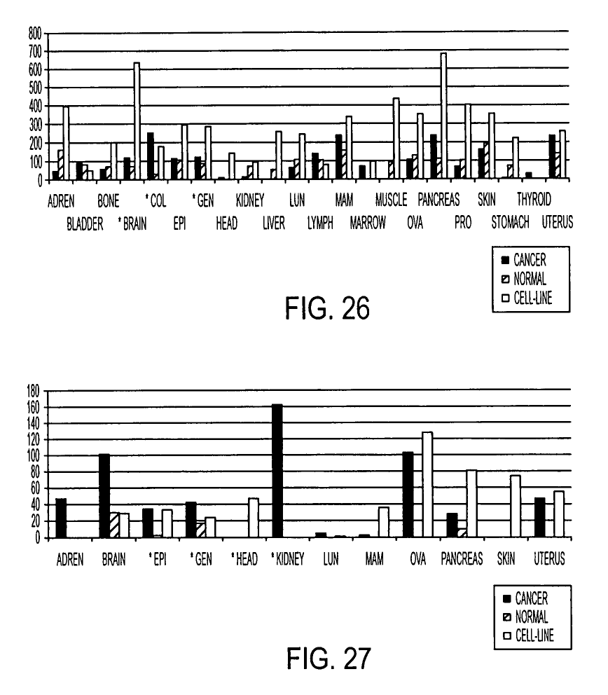

Overall, the following results were obtained as shown with regard to the histograms in FIG. 26 and Table 4. This cluster is overexpressed (at least at a minimum level) in the following pathological conditions: brain malignant tumors, colorectal cancer and a mixture of malignant tumors from different tissues.

TABLE 4
Normal tissue distribution

| Name of Tissue | Number |
|---|---|
| adrenal | 160 |
| bladder | 82 |
| Bone | 71 |
| brain | 70 |
| colon | 31 |
| epithelial | 106 |
| general | 88 |
| head and neck | 0 |
| kidney | 71 |
| liver | 53 |
| lung | 108 |
| lymph nodes | 107 |
| breast | 158 |
| bone marrow | 0 |
| muscle | 94 |
| ovary | 131 |
| pancreas | 113 |
| prostate | 106 |
| skin | 193 |
| stomach | 73 |
| Thyroid | 0 |
| uterus | 140 |

TABLE 5
P values and ratios for expression in cancerous tissue

| Name of Tissue | P1 | P2 | SP1 | R3 | SP2 | R4 |
|---|---|---|---|---|---|---|
| adrenal | 7.4e−01 | 6.9e−01 | 9.5e−01 | 0.4 | 7.1e−01 | 0.7 |
| bladder | 7.0e−01 | 6.6e−01 | 6.2e−01 | 1.1 | 7.1e−01 | 1.0 |
| bone | 4.9e−01 | 6.3e−01 | 7.9e−01 | 0.9 | 2.1e−01 | 1.1 |
| brain | 7.4e−02 | 2.0e−02 | 1.0e−01 | 1.5 | 5.2e−12 | 3.2 |
| colon | 2.6e−03 | 5.7e−04 | 9.9e−03 | 4.3 | 1.3e−02 | 4.0 |
| epithelial | 3.2e−01 | 2.5e−02 | 3.3e−01 | 1.0 | 9.1e−08 | 1.8 |
| general | 4.9e−02 | 2.3e−04 | 3.6e−03 | 1.3 | 1.4e−23 | 2.2 |
| head and neck | 2.1e−01 | 1.1e−01 | 1 | 1.1 | 4.2e−01 | 2.0 |
| kidney | 8.6e−01 | 8.8e−01 | 9.7e−01 | 0.4 | 8.1e−01 | 0.6 |
| liver | 5.2e−01 | 9.1e−02 | 1 | 0.5 | 1.0e−01 | 2.4 |
| lung | 7.1e−01 | 7.3e−01 | 8.9e−01 | 0.6 | 1.3e−01 | 0.9 |
| lymph nodes | 5.9e−01 | 6.6e−01 | 4.1e−01 | 1.3 | 6.0e−01 | 0.9 |
| breast | 1.9e−01 | 1.5e−01 | 2.6e−01 | 1.0 | 1.4e−01 | 1.3 |
| bone marrow | 4.3e−01 | 2.5e−01 | 1 | 3.3 | 1.5e−01 | 4.0 |
| muscle | 6.7e−01 | 5.1e−01 | 1 | 0.2 | 1.6e−02 | 0.7 |
| ovary | 6.3e−01 | 4.9e−01 | 6.9e−01 | 0.9 | 2.8e−01 | 1.0 |
| pancreas | 2.2e−01 | 1.7e−01 | 3.8e−02 | 0.9 | 5.5e−05 | 1.6 |
| prostate | 8.7e−01 | 8.3e−01 | 8.3e−01 | 0.7 | 1.8e−01 | 1.0 |
| skin | 5.0e−01 | 4.9e−01 | 6.9e−01 | 0.8 | 3.3e−02 | 0.9 |
| stomach | 5.2e−01 | 3.9e−01 | 1 | 0.4 | 2.0e−01 | 1.5 |
| Thyroid | 4.6e−01 | 4.6e−01 | 1 | 1.2 | 1 | 1.2 |
| uterus | 6.1e−01 | 3.6e−01 | 1.7e−01 | 1.0 | 1.2e−01 | 1.3 |

As noted above, cluster D11853 features 18 transcript(s), which were listed in Table 1 above. These transcript(s) encode for protein(s) which are variant(s) of protein Membrane associated protein SLP-2 (SEQ ID NO:637). A description of each variant protein according to the present invention is now provided.

Variant protein D11853_PEA_{—}1_P1 (SEQ ID NO:588) according to the present invention has an amino acid sequence as given at the end of the application; it is encoded by transcript(s) D11853_PEA_{—}1_T1 (SEQ ID NO:57). An alignment is given to the known protein (Membrane associated protein SLP-2 (SEQ ID NO:637)) at the end of the application. One or more alignments to one or more previously published protein sequences are given at the end of the application. A brief description of the relationship of the variant protein according to the present invention to each such aligned protein is as follows:

Comparison Report Between D11853_PEA_{—}1_P1 (SEQ ID NO:588) and Q9P042 (SEQ ID NO 639):

1. An isolated chimeric polypeptide encoding for D11853_PEA_{—}1_P1 (SEQ ID NO:588), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPR (SEQ ID NO:1530) corresponding to amino acids 1-26 of D11853_PEA_{—}1_P1 (SEQ ID NO:588), a second amino acid sequence being at least 90% homologous to RASSGLPRNTVVLFVPQQEAWVVERMGRFHRILEPGLNILIPVLDRIRYVQSLKEIVINVPEQSAVTLDNVT LQIDGVLYLRIMDPYKASYGVEDPEYAVTQLAQTTMRSELGKLSLDKVFRERESLNASIVDAINQAADCW GIRCLRYEIKDIHVPPRVKESMQMQVEAERRKR corresponding to amino acids 13-187 of Q9P042 (SEQ ID NO:639), which also corresponds to amino acids 27-201 of D11853_PEA_{—}1_P1 (SEQ ID NO:588), a bridging amino acid A corresponding to amino acid 202 of D11853_PEA_{—}1_P1 (SEQ ID NO:588), and a third amino acid sequence being at least 90% homologous to TVLESEGTRESAINVAEGKKQAQILASEAEKAEQINQAAGEASAVLAKAKAKAEAIRILAAALTQHNGDA AASLTVAEQYVSAFSKLAKDSNTILLPSNPGDVTSMVAQAMGVYGALTKAPVPGTPDSLSSGSSRDVQGT DASLDEELDRVKMS corresponding to amino acids 189-342 of Q9P042 (SEQ ID NO:639), which also corresponds to amino acids 203-356 of D11853_PEA_{—}1_P1 (SEQ ID NO:588), wherein said first amino acid sequence, second amino acid sequence, bridging amino acid and third amino acid sequence are contiguous and in a sequential order.

2. An isolated polypeptide encoding for a head of D11853_PEA_{—}1_P1 (SEQ ID NO:588), comprising a polypeptide being at least 70%, optionally at least about 80%, preferably at least about 85%, more preferably at least about 90% and most preferably at least about 95% homologous to the sequence MLARAARGTGALLLRGSLLASGRAPR (SEQ ID NO:1530) of D11853_PEA_{—}1_P1 (SEQ ID NO:588).

Comparison Report Between D11853_PEA_{—}1_P1 (SEQ ID NO:588) and BAC85377 (SEQ ID NO 640):

1. An isolated chimeric polypeptide encoding for D11853_PEA_{—}1_P1 (SEQ ID NO:588), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRI corresponding to amino acids 1-109 of D11853_PEA_{—}1_P1 (SEQ ID NO:588), a second amino acid sequence being at least 90% homologous to MDPYKASYGVEDPEYAVTQLAQTTMRSELGKLSLDKVFRERESLNASIVDAINQAADCWGIRCLRYEIKD IHVPPRVKESMQMQVEAERRKRATVLESEGTRESAINVAEGKKQAQILASEAEKAEQINQAAGEASAVLA KAKAKAEAIRILAAALTQH corresponding to amino acids 1-159 of BAC85377 (SEQ ID NO:640), which also corresponds to amino acids 110-268 of D11853_PEA_{—}1_P1 (SEQ ID NO:588), and a third amino acid sequence being at least 70%, optionally at least 80%, preferably at least 85%, more preferably at least 90% and most preferably at least 95% homologous to a polypeptide having the sequence NGDAAASLTVAEQYVSAFSKLAKDSNTILLPSNPGDVTSMVAQAMGVYGALTKAPVPGTPDSLSSGSSRD VQGTDASLDEELDRVKMS corresponding to amino acids 269-356 of D11853_PEA_{—}1_P1 (SEQ ID NO:588), wherein said first amino acid sequence, second amino acid sequence and third amino acid sequence are contiguous and in a sequential order.

2. An isolated polypeptide encoding for a head of D11853_PEA_{—}1_P1 (SEQ ID NO:588), comprising a polypeptide being at least 70%, optionally at least about 80%, preferably at least about 85%, more preferably at least about 90% and most preferably at least about 95% homologous to the sequence MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRI of D11853_PEA_{—}1_P1 (SEQ ID NO:588).

3. An isolated polypeptide encoding for a tail of D11853_PEA_{—}1_P1 (SEQ ID NO:588), comprising a polypeptide being at least 70%, optionally at least about 80%, preferably at least about 85%, more preferably at least about 90% and most preferably at least about 95% homologous to the sequence NGDAAASLTVAEQYVSAFSKLAKDSNTILLPSNPGDVTSMVAQAMGVYGALTKAPVPGTPDSLSSGSSRD VQGTDASLDEELDRVKMS in D11853_PEA_{—}1_P1 (SEQ ID NO:588).

Comparison Report Between D11853_PEA_{—}1_P1 (SEQ ID NO:588) and Q96FY2 (SEQ ID NO: 638):

1. An isolated chimeric polypeptide encoding for D11853_PEA_{—}1_P1 (SEQ ID NO:588), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQ corresponding to amino acids 1-128 of Q96FY2 (SEQ ID NO:638), which also corresponds to amino acids 1-128 of D11853_PEA_{—}1_P1 (SEQ ID NO:588), a bridging amino acid L corresponding to amino acid 129 of D11853_PEA_{—}1_P1 (SEQ ID NO:588), and a second amino acid sequence being at least 90% homologous to AQTTMRSELGKLSLDKVFRERESLNASIVDAINQAADCWGIRCLRYEIKDIHVPPRVKESMQMQVEAERR KRATVLESEGTRESAINVAEGKKQAQILASEAEKAEQINQAAGEASAVLAKAKAKAEAIRILAAALTQHN GDAAASLTVAEQYVSAFSKLAKDSNTILLPSNPGDVTSMVAQAMGVYGALTKAPVPGTPDSLSSGSSRDV QGTDASLDEELDRVKMS corresponding to amino acids 130-356 of Q96FY2 (SEQ ID NO:638), which also corresponds to amino acids 130-356 of D11853_PEA_{—}1_P1 (SEQ ID NO:588), wherein said first amino acid sequence, bridging amino acid and second amino acid sequence are contiguous and in a sequential order.

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P1 (SEQ ID NO:588) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein D11853_PEA_{—}1_P1 (SEQ ID NO:588) sequence provides support for the deduced sequence of this variant protein according to the present invention).

TABLE 6
Amino acid mutations

| SNP position(s) on amino acid | Alternative | |
|---|---|---|
| sequence | amino acid(s) | Previously known SNP? |
| 25 | P -> | No |
| 32 | L -> | No |
| 178 | K -> | No |
| 178 | K -> R | No |
| 178 | K -> T | No |
| 185 | R -> G | No |
| 187 | K -> T | No |
| 206 | E -> K | No |
| 230 | E -> K | No |
| 230 | E -> Q | No |
| 235 | E -> | No |
| 235 | E -> D | No |
| 239 | Q -> P | No |
| 244 | A -> G | No |
| 244 | A -> | No |
| 267 | Q -> H | No |
| 278 | V -> G | No |
| 284 | S -> N | No |
| 284 | S -> T | No |
| 299 | P -> | No |
| 299 | P -> A | No |
| 326 | G -> | No |
| 329 | D -> N | No |
| 340 | Q -> | No |

Variant protein D11853_PEA_{—}1_P1 (SEQ ID NO:588) is encoded by the following transcript(s): D11853_PEA_{—}1_T1 (SEQ ID NO:57), for which the sequence(s) is/are given at the end of the application. The coding portion of transcript D11853_PEA_{—}1_T1 (SEQ ID NO:57) is shown in bold; this coding portion starts at position 108 and ends at position 1175. The transcript also has the following SNPs as listed in Table 7 (given according to their position on the nucleotide sequence, with the alternative nucleic acid listed; the last column indicates whether the SNP is known or not; the presence of known SNPs in variant protein D11853_PEA_{—}1_P1 (SEQ ID NO:588) sequence provides support for the deduced sequence of this variant protein according to the present invention).

TABLE 7
Nucleic acid SNPs

| SNP position on | Alternative | Previously known |
|---|---|---|
| nucleotide sequence | nucleic acid | SNP? |
| 180 | C -> | No |
| 182 | G -> | No |
| 185 | -> C | No |
| 201 | T -> | No |
| 640 | A -> C | No |
| 640 | A -> G | No |
| 641 | G -> | No |
| 641 | G -> A | No |
| 660 | C -> G | No |
| 667 | A -> C | No |
| 723 | G -> A | No |
| 795 | G -> A | No |
| 795 | G -> C | No |
| 812 | A -> | No |
| 812 | A -> C | No |
| 823 | A -> C | No |
| 838 | C -> | No |
| 838 | C -> G | No |
| 908 | A -> C | No |
| 932 | A -> C | No |
| 940 | T -> G | No |
| 958 | G -> A | No |
| 958 | G -> C | No |
| 966 | -> C | No |
| 966 | -> T | No |
| 973 | -> G | No |
| 1002 | C -> | No |
| 1002 | C -> G | No |
| 1033 | -> G | No |
| 1083 | G -> | No |
| 1092 | G -> A | No |
| 1112 | C -> T | No |
| 1127 | G -> | No |
| 1208 | G -> A | No |
| 1211 | A -> | No |
| 1211 | A -> C | No |
| 1257 | T -> | No |
| 1260 | T -> C | No |
| 1260 | T -> G | No |
| 1297 | T -> C | Yes |

Variant protein D11853_PEA_{—}1_P2 (SEQ ID NO:589) according to the present invention has an amino acid sequence as given at the end of the application; it is encoded by transcript(s) D11853_PEA_{—}1_T3 (SEQ ID NO:58). An alignment is given to the known protein (Membrane associated protein SLP-2 (SEQ ID NO:637)) at the end of the application. One or more alignments to one or more previously published protein sequences are given at the end of the application. A brief description of the relationship of the variant protein according to the present invention to each such aligned protein is as follows:

Comparison Report Between D11853_PEA_{—}1_P2 (SEQ ID NO:589) and Q9P042 (SEQ ID NO: 639):

1. An isolated chimeric polypeptide encoding for D11853_PEA_{—}1_P2 (SEQ ID NO:589), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPR (SEQ ID NO:1530) corresponding to amino acids 1-26 of D11853_PEA_{—}1_P2 (SEQ ID NO:589), a second amino acid sequence being at least 90% homologous to RASSGLPRNTVVLFVPQQEAWVVERMGRFHRILEPGLNILIPVLDRIRYVQSLKEIVINVPEQSAVTLDNVT LQIDGVLYLRIMDPYKASYGVEDPEYAVTQLAQTTMRSELGKLSLDKVFRERESLNASIVDAINQAADCW GIRCLRYEIKDIHVPPRVKESMQMQVEAERRKR corresponding to amino acids 13-187 of Q9P042 (SEQ ID NO:639), which also corresponds to amino acids 27-201 of D11853_PEA_{—}1_P2 (SEQ ID NO:589), a bridging amino acid A corresponding to amino acid 202 of D11853_PEA_{—}1_P2 (SEQ ID NO:589), a third amino acid sequence being at least 90% homologous to TVLESEGTRESAINVAEGKKQAQILASEAEKAEQINQAAGEASAVLAKAKAKAEAIRILAAALTQHNGDA AASLTVAEQYVSAFSKLAKDSNTILLPSNPGDVTSMVAQ corresponding to amino acids 189-297 of Q9P042 (SEQ ID NO:639), which also corresponds to amino acids 203-311 of D11853_PEA_{—}1_P2 (SEQ ID NO:589) and a fourth amino acid sequence being at least 70%, optionally at least 80%, preferably at least 85%, more preferably at least 90% and most preferably at least 95% homologous to a polypeptide having the sequence VRAL (SEQ ID NO:1533) corresponding to amino acids 312-315 of D11853_PEA_{—}1_P2 (SEQ ID NO:589), wherein said first amino acid sequence, second amino acid sequence, bridging amino acid, third amino acid sequence and fourth amino acid sequence are contiguous and in a sequential order.

2. An isolated polypeptide encoding for a head of D11853_PEA_{—}1_P2 (SEQ ID NO:589), comprising a polypeptide being at least 70%, optionally at least about 80%, preferably at least about 85%, more preferably at least about 90% and most preferably at least about 95% homologous to the sequence MLARAARGTGALLLRGSLLASGRAPR (SEQ ID NO:1530) of D11853_PEA_{—}1_P2 (SEQ ID NO:589).

3. An isolated polypeptide encoding for a tail of D11853_PEA_{—}1_P2 (SEQ ID NO:589), comprising a polypeptide being at least 70%, optionally at least about 80%, preferably at least about 85%, more preferably at least about 90% and most preferably at least about 95% homologous to the sequence VRAL (SEQ ID NO:1533) in D11853_PEA_{—}1_P2 (SEQ ID NO:589).

Comparison Report Between D11853_PEA_{—}1_P2 (SEQ ID NO:589) and BAC85377 (SEQ ID NO: 640):

1. An isolated chimeric polypeptide encoding for D11853_PEA_{—}1_P2 (SEQ ID NO:589), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRI corresponding to amino acids 1-109 of D11853_PEA_{—}1_P2 (SEQ ID NO:589), a second amino acid sequence being at least 90% homologous to MDPYKASYGVEDPEYAVTQLAQTTMRSELGKLSLDKVFRERESLNASIVDAINQAADCWGIRCLRYEIKD IHVPPRVKESMQMQVEAERRKRATVLESEGTRESAINVAEGKKQAQILASEAEKAEQINQAAGEASAVLA KAKAKAEAIRILAAALTQH corresponding to amino acids 1-159 of BAC85377 (SEQ ID NO:640), which also corresponds to amino acids 110-268 of D11853_PEA_{—}1_P2 (SEQ ID NO:589), and a third amino acid sequence being at least 70%, optionally at least 80%, preferably at least 85%, more preferably at least 90% and most preferably at least 95% homologous to a polypeptide having the sequence NGDAAASLTVAEQYVSAFSKLAKDSNTILLPSNPGDVTSMVAQVRAL corresponding to amino acids 269-315 of D11853_PEA_{—}1_P2 (SEQ ID NO:589), wherein said first amino acid sequence, second amino acid sequence and third amino acid sequence are contiguous and in a sequential order.

2. An isolated polypeptide encoding for a head of D11853_PEA_{—}1_P2 (SEQ ID NO:589), comprising a polypeptide being at least 70%, optionally at least about 80%, preferably at least about 85%, more preferably at least about 90% and most preferably at least about 95% homologous to the sequence MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRI of D11853_PEA_{—}1_P2 (SEQ ID NO:589).

3. An isolated polypeptide encoding for a tail of D11853_PEA_{—}1_P2 (SEQ ID NO:589), comprising a polypeptide being at least 70%, optionally at least about 80%, preferably at least about 85%, more preferably at least about 90% and most preferably at least about 95% homologous to the sequence NGDAAASLTVAEQYVSAFSKLAKDSNTILLPSNPGDVTSMVAQVRAL in D11853_PEA_{—}1_P2 (SEQ ID NO:589).

Comparison Report Between D11853_PEA_{—}1_P2 (SEQ ID NO:589) and Q96FY2 (SEQ ID NO:638):

1. An isolated chimeric polypeptide encoding for D11853_PEA_{—}1_P2 (SEQ ID NO:589), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQ corresponding to amino acids 1-128 of Q96FY2 (SEQ ID NO:638), which also corresponds to amino acids 1-128 of D11853_PEA_{—}1_P2 (SEQ ID NO:589), a bridging amino acid L corresponding to amino acid 129 of D11853_PEA_{—}1_P2 (SEQ ID NO:589), a second amino acid sequence being at least 90% homologous to AQTTMRSELGKLSLDKVFRERESLNASIVDAINQAADCWGIRCLRYEIKDIHVPPRVKESMQMQVEAERR KRATVLESEGTRESAINVAEGKKQAQILASEAEKAEQINQAAGEASAVLAKAKAKAEAIRILAAALTQHN GDAAASLTVAEQYVSAFSKLAKDSNTILLPSNPGDVTSMVAQ corresponding to amino acids 130-311 of Q96FY2 (SEQ ID NO:638), which also corresponds to amino acids 130-311 of D11853_PEA_{—}1_P2 (SEQ ID NO:589), and a third amino acid sequence being at least 70%, optionally at least 80%, preferably at least 85%, more preferably at least 90% and most preferably at least 95% homologous to a polypeptide having the sequence VRAL (SEQ ID NO:1533) corresponding to amino acids 312-315 of D11853_PEA_{—}1_P2 (SEQ ID NO:589), wherein said first amino acid sequence, bridging amino acid, second amino acid sequence and third amino acid sequence are contiguous and in a sequential order.

2. An isolated polypeptide encoding for a tail of D11853_PEA_{—}1_P2 (SEQ ID NO:589), comprising a polypeptide being at least 70%, optionally at least about 80%, preferably at least about 85%, more preferably at least about 90% and most preferably at least about 95% homologous to the sequence VRAL (SEQ ID NO:1533) in D11853_PEA_{—}1_P2 (SEQ ID NO:589).

Comparison Report Between D11853_PEA_{—}1_P2 (SEQ ID NO:589) and Q9UJZ1 (SEQ ID NO:637):

1. An isolated chimeric polypeptide encoding for D11853_PEA_{—}1_P2 (SEQ ID NO:589), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQLAQTTMRSELGKLS LDKVFRERESLNASIVDAINQAADCWGIRCLRYEIKDIHVPPRVKESMQMQVEAERRKRATVLESEGTRES AINVAEGKKQAQILASEAEKAEQINQAAGEASAVLAKAKAKAEAIRILAAALTQIINGDAAASLTVAEQY VSAFSKLAKDSNTILLPSNPGDVTSMVAQ corresponding to amino acids 1-311 of Q9UJZ1 (SEQ ID NO:637), which also corresponds to amino acids 1-311 of D11853_PEA_{—}1_P2 (SEQ ID NO:589), and a second amino acid sequence being at least 70%, optionally at least 80%, preferably at least 85%, more preferably at least 90% and most preferably at least 95% homologous to a polypeptide having the sequence VRAL (SEQ ID NO:1533) corresponding to amino acids 312-315 of D11853_PEA_{—}1_P2 (SEQ ID NO:589), wherein said first amino acid sequence and second amino acid sequence are contiguous and in a sequential order.

2. An isolated polypeptide encoding for a tail of D11853_PEA_{—}1_P2 (SEQ ID NO:589), comprising a polypeptide being at least 70%, optionally at least about 80%, preferably at least about 85%, more preferably at least about 90% and most preferably at least about 95% homologous to the sequence VRAL (SEQ ID NO:1533) in D11853_PEA_{—}1_P2 (SEQ ID NO:589).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P2 (SEQ ID NO:589) also has the following non-silent SNPs (Single Nucleotide Polymorphisms) as listed in Table 8, (given according to their position(s) on the amino acid sequence, with the alternative amino acid(s) listed; the last column indicates whether the SNP is known or not; the presence of known SNPs in variant protein D11853_PEA_{—}1_P2 (SEQ ID NO:589) sequence provides support for the deduced sequence of this variant protein according to the present invention).

TABLE 8
Amino acid mutations

| SNP position(s) on amino acid | Alternative | |
|---|---|---|
| sequence | amino acid(s) | Previously known SNP? |
| 25 | P -> | No |
| 32 | L -> | No |
| 178 | K -> | No |
| 178 | K -> R | No |
| 178 | K -> T | No |
| 185 | R -> G | No |
| 187 | K -> T | No |
| 206 | E -> K | No |
| 230 | E -> Q | No |
| 230 | E -> K | No |
| 235 | E -> D | No |
| 235 | E -> | No |
| 239 | Q -> P | No |
| 244 | A -> | No |
| 244 | A -> G | No |
| 267 | Q -> H | No |
| 278 | V -> G | No |
| 284 | S -> T | No |
| 284 | S -> N | No |
| 299 | P -> A | No |
| 299 | P -> | No |

Variant protein D11853_PEA_{—}1_P2 (SEQ ID NO:589) is encoded by the following transcript(s): D11853_PEA_{—}1_T3 (SEQ ID NO:58), for which the sequence(s) is/are given at the end of the application. The coding portion of transcript D11853_PEA_{—}1_T3 (SEQ ID NO:58) is shown in bold; this coding portion starts at position 108 and ends at position 1052. The transcript also has the following SNPs as listed in Table 9 (given according to their position on the nucleotide sequence, with the alternative nucleic acid listed; the last column indicates whether the SNP is known or not; the presence of known SNPs in variant protein D11853_PEA_{—}1_P2 (SEQ ID NO:589) sequence provides support for the deduced sequence of this variant protein according to the present invention).

TABLE 9
Nucleic acid SNPs

| SNP position on | Alternative | Previously known |
|---|---|---|
| nucleotide sequence | nucleic acid | SNP? |
| 180 | C -> | No |
| 182 | G -> | No |
| 185 | -> C | No |
| 201 | T -> | No |
| 640 | A -> C | No |
| 640 | A -> G | No |
| 641 | G -> | No |
| 641 | G -> A | No |
| 660 | C -> G | No |
| 667 | A -> C | No |
| 723 | G -> A | No |
| 795 | G -> A | No |
| 795 | G -> C | No |
| 812 | A -> | No |
| 812 | A -> C | No |
| 823 | A -> C | No |
| 838 | C -> | No |
| 838 | C -> G | No |
| 908 | A -> C | No |
| 932 | A -> C | No |
| 940 | T -> G | No |
| 958 | G -> A | No |
| 958 | G -> C | No |
| 966 | -> C | No |
| 966 | -> T | No |
| 973 | -> G | No |
| 1002 | C -> | No |
| 1002 | C -> G | No |
| 1033 | -> G | No |
| 1066 | C -> T | No |
| 1508 | G -> | No |
| 1517 | G -> A | No |
| 1537 | C -> T | No |
| 1552 | G -> | No |
| 1633 | G -> A | No |
| 1636 | A -> | No |
| 1636 | A -> C | No |
| 1682 | T -> | No |
| 1685 | T -> C | No |
| 1685 | T -> G | No |
| 1722 | T -> C | Yes |

Variant protein D11853_PEA_{—}1_P7 (SEQ ID NO:590) according to the present invention has an amino acid sequence as given at the end of the application; it is encoded by transcript(s) D11853_PEA_{—}1_T10 (SEQ ID NO:62). An alignment is given to the known protein (Membrane associated protein SLP-2 (SEQ ID NO:637)) at the end of the application. One or more alignments to one or more previously published protein sequences are given at the end of the application. A brief description of the relationship of the variant protein according to the present invention to each such aligned protein is as follows:

Comparison Report Between D11853_PEA_{—}1_P7 (SEQ ID NO:590) and Q9P042 (SEQ ID NO:639):

1. An isolated chimeric polypeptide encoding for D11853_PEA_{—}1_P7 (SEQ ID NO:590), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPR (SEQ ID NO:1530) corresponding to amino acids 1-26 of D11853_PEA_{—}1_P7 (SEQ ID NO:590), a second amino acid sequence being at least 90% homologous to RASSGLPRNTVVLFVPQQEAWVVERMGRFHRILEPGLNILIPVLDRIRYVQSLKEIVINVPEQSAVTLDNVT LQIDGVLYLRIMDPYKASYGVEDPEYAVTQLAQTTMRSELGKLSLDKVFRERESLNASIVDAINQAADCW GIRCLRYEIKDIHVPPRVKESMQMQVEAERRKR corresponding to amino acids 13-187 of Q9P042 (SEQ ID NO:639), which also corresponds to amino acids 27-201 of D11853_PEA_{—}1_P7 (SEQ ID NO:590), a bridging amino acid A corresponding to amino acid 202 of D11853_PEA_{—}1_P7 (SEQ ID NO:590), a third amino acid sequence being at least 90% homologous to TVLESEGTRESAINVAEGKKQAQILASEAEKAEQINQAAGEASAVLAKAKAKAEAIRILAAALTQH corresponding to amino acids 189-254 of Q9P042 (SEQ ID NO:639), which also corresponds to amino acids 203-268 of D11853_PEA_{—}1_P7 (SEQ ID NO:590), and a fourth amino acid sequence being at least 70%, optionally at least 80%, preferably at least 85%, more preferably at least 90% and most preferably at least 95% homologous to a polypeptide having the sequence VRGPWVGMGTGIDSGRGSLIYA (SEQ ID NO:1535) corresponding to amino acids 269-290 of D11853_PEA_{—}1_P7 (SEQ ID NO:590), wherein said first amino acid sequence, second amino acid sequence, bridging amino acid, third amino acid sequence and fourth amino acid sequence are contiguous and in a sequential order.

2. An isolated polypeptide encoding for a head of D11853_PEA_{—}1_P7 (SEQ ID NO:590), comprising a polypeptide being at least 70%, optionally at least about 80%, preferably at least about 85%, more preferably at least about 90% and most preferably at least about 95% homologous to the sequence MLARAARGTGALLLRGSLLASGRAPR (SEQ ID NO:1530) of D11853_PEA_{—}1_P7 (SEQ ID NO:590).

3. An isolated polypeptide encoding for a tail of D11853_PEA_{—}1_P7 (SEQ ID NO:590), comprising a polypeptide being at least 70%, optionally at least about 80%, preferably at least about 85%, more preferably at least about 90% and most preferably at least about 95% homologous to the sequence VRGPWVGMGTGIDSGRGSLIYA (SEQ ID NO:1535) in D11853_PEA_{—}1_P7 (SEQ ID NO:590).

Comparison Report Between D11853_PEA_{—}1_P7 (SEQ ID NO:590) and BAC85377 (SEQ ID NO:640):

1. An isolated chimeric polypeptide encoding for D11853_PEA_{—}1_P7 (SEQ ID NO:590), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRI corresponding to amino acids 1-109 of D11853_PEA_{—}1_P7 (SEQ ID NO:590), and a second amino acid sequence being at least 90% homologous to MDPYKASYGVEDPEYAVTQLAQTTMRSELGKLSLDKVFRERESLNASIVDAINQAADCWGIRCLRYEIKD IHVPPRVKESMQMQVEAERRKRATVLESEGTRESAINVAEGKKQAQILASEAEKAEQINQAAGEASAVLA KAKAKAEAIRILAAALTQHVRGPWVGMGTGIDSGRGSLIYA corresponding to amino acids 1-181 of BAC85377 (SEQ ID NO:640), which also corresponds to amino acids 110-290 of D11853_PEA_{—}1_P7 (SEQ ID NO:590), wherein said first amino acid sequence and second amino acid sequence are contiguous and in a sequential order.

2. An isolated polypeptide encoding for a head of D11853_PEA_{—}1_P7 (SEQ ID NO:590), comprising a polypeptide being at least 70%, optionally at least about 80%, preferably at least about 85%, more preferably at least about 90% and most preferably at least about 95% homologous to the sequence MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRI of D11853_PEA_{—}1_P7 (SEQ ID NO:590).

Comparison Report Between D11853_PEA_{—}1_P7 (SEQ ID NO:590) and Q96FY2 (SEQ ID NO 638):

1. An isolated chimeric polypeptide encoding for D11853_PEA_{—}1_P7 (SEQ ID NO:590), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQ corresponding to amino acids 1-128 of Q96FY2 (SEQ ID NO:638), which also corresponds to amino acids 1-128 of D11853_PEA_{—}1_P7 (SEQ ID NO:590), a bridging amino acid L corresponding to amino acid 129 of D11853_PEA_{—}1_P7 (SEQ ID NO:590), a second amino acid sequence being at least 90% homologous to AQTTMRSELGKLSLDKVFRERESLNASIVDAINQAADCWGIRCLRYEIKDIHVPPRVKESMQMQVEAERR KRATVLESEGTRESAINVAEGKKQAQILASEAEKAEQINQAAGEASAVLAKAKAKAEAIRILAAALTQH corresponding to amino acids 130-268 of Q96FY2 (SEQ ID NO:638), which also corresponds to amino acids 130-268 of D11853_PEA_{—}1_P7 (SEQ ID NO:590), and a third amino acid sequence being at least 70%, optionally at least 80%, preferably at least 85%, more preferably at least 90% and most preferably at least 95% homologous to a polypeptide having the sequence VRGPWVGMGTGIDSGRGSLIYA (SEQ ID NO:1535) corresponding to amino acids 269-290 of D11853_PEA_{—}1_P7 (SEQ ID NO:590), wherein said first amino acid sequence, bridging amino acid, second amino acid sequence and third amino acid sequence are contiguous and in a sequential order.

2. An isolated polypeptide encoding for a tail of D11853_PEA_{—}1_P7 (SEQ ID NO:590), comprising a polypeptide being at least 70%, optionally at least about 80%, preferably at least about 85%, more preferably at least about 90% and most preferably at least about 95% homologous to the sequence VRGPWVGMGTGIDSGRGSLIYA (SEQ ID NO:1535) in D11853_PEA_{—}1_P7 (SEQ ID NO:590).

Comparison Report Between D11853_PEA_{—}1_P7 (SEQ ID NO:590) and Q9UJZ1 (SEQ ID NO:637):

1. An isolated chimeric polypeptide encoding for D11853_PEA_{—}1_P7 (SEQ ID NO:590), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQLAQTTMRSELGKLS LDKVFRERESLNASIVDAINQAADCWGIRCLRYEIKDIHVPPRVKESMQMQVEAERRKRATVLESEGTRES AINVAEGKKQAQILASEAEKAEQINQAAGEASAVLAKAKAKAEAIRILAAALTQH corresponding to amino acids 1-268 of Q9UJZ1 (SEQ ID NO:637), which also corresponds to amino acids 1-268 of D11853_PEA_{—}1_P7 (SEQ ID NO:590), and a second amino acid sequence being at least 70%, optionally at least 80%, preferably at least 85%, more preferably at least 90% and most preferably at least 95% homologous to a polypeptide having the sequence VRGPWVGMGTGIDSGRGSLIYA (SEQ ID NO:1535) corresponding to amino acids 269-290 of D11853_PEA_{—}1_P7 (SEQ ID NO:590), wherein said first amino acid sequence and second amino acid sequence are contiguous and in a sequential order.

2. An isolated polypeptide encoding for a tail of D11853_PEA_{—}1_P7 (SEQ ID NO:590), comprising a polypeptide being at least 70%, optionally at least about 80%, preferably at least about 85%, more preferably at least about 90% and most preferably at least about 95% homologous to the sequence VRGPWVGMGTGIDSGRGSLIYA (SEQ ID NO:1535) in D11853_PEA_{—}1_P7 (SEQ ID NO:590).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM: Signal peptide,NN:NO) predicts that this protein has a signal peptide.

Variant protein D11853_PEA_{—}1_P7 (SEQ ID NO:590) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein D11853_PEA_{—}1_P7 (SEQ ID NO:590) sequence provides support for the deduced sequence of this variant protein according to the present invention).

TABLE 10
Amino acid mutations

| SNP position(s) on | Alternative | Previously |
|---|---|---|
| amino acid sequence | amino acid(s) | known SNP? |
| 25 | P -> | No |
| 32 | L -> | No |
| 178 | K -> | No |
| 178 | K -> R | No |
| 178 | K -> T | No |
| 185 | R -> G | No |
| 187 | K -> T | No |
| 206 | E -> K | No |
| 230 | E -> K | No |
| 230 | E -> Q | No |
| 235 | E -> | No |
| 235 | E -> D | No |
| 239 | Q -> P | No |
| 244 | A -> | No |
| 244 | A -> G | No |
| 267 | Q -> H | No |

Variant protein D11853_PEA_{—}1_P7 (SEQ ID NO:590) is encoded by the following transcript(s): D11853_PEA_{—}1_T10 (SEQ ID NO:62), for which the sequence(s) is/are given at the end of the application. The coding portion of transcript D11853_PEA_{—}1_T10 (SEQ ID NO:62) is shown in bold; this coding portion starts at position 108 and ends at position 977. The transcript also has the following SNPs as listed in Table 11 (given according to their position on the nucleotide sequence, with the alternative nucleic acid listed; the last column indicates whether the SNP is known or not; the presence of known SNPs in variant protein D11853_PEA_{—}1_P7 (SEQ ID NO:590) sequence provides support for the deduced sequence of this variant protein according to the present invention).

TABLE 11
Nucleic acid SNPs

| SNP position on | Alternative | Previously |
|---|---|---|
| nucleotide sequence | nucleic acid | known SNP? |
| 180 | C -> | No |
| 182 | G -> | No |
| 185 | -> C | No |
| 201 | T -> | No |
| 640 | A -> C | No |
| 640 | A -> G | No |
| 641 | G -> | No |
| 641 | G -> A | No |
| 660 | C -> G | No |
| 667 | A -> C | No |
| 723 | G -> A | No |
| 795 | G -> A | No |
| 795 | G -> C | No |
| 812 | A -> | No |
| 812 | A -> C | No |
| 823 | A -> C | No |
| 838 | C -> | No |
| 838 | C -> G | No |
| 908 | A -> C | No |
| 1137 | A -> C | No |
| 1145 | T -> G | No |
| 1163 | G -> A | No |
| 1163 | G -> C | No |
| 1171 | -> C | No |
| 1171 | -> T | No |
| 1178 | -> G | No |
| 1207 | C -> | No |
| 1207 | C -> G | No |
| 1238 | -> G | No |
| 1288 | G -> | No |
| 1297 | G -> A | No |
| 1317 | C -> T | No |
| 1332 | G -> | No |
| 1413 | G -> A | No |
| 1416 | A -> | No |
| 1416 | A -> C | No |
| 1462 | T -> | No |
| 1465 | T -> C | No |
| 1465 | T -> G | No |
| 1502 | T -> C | Yes |

Variant protein D11853_PEA_{—}1_P9 (SEQ ID NO:591) according to the present invention has an amino acid sequence as given at the end of the application; it is encoded by transcript(s) D11853_PEA_{—}1_T13 (SEQ ID NO:63). An alignment is given to the known protein (Membrane associated protein SLP-2 (SEQ ID NO:637)) at the end of the application. One or more alignments to one or more previously published protein sequences are given at the end of the application. A brief description of the relationship of the variant protein according to the present invention to each such aligned protein is as follows:

Comparison Report Between D11853_PEA_{—}1_P9 (SEQ ID NO:591) and Q9P042 (SEQ ID NO:639):

1. An isolated chimeric polypeptide encoding for D11853_PEA_{—}1_P9 (SEQ ID NO:591), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPR (SEQ ID NO:1530) corresponding to amino acids 1-26 of D11853_PEA_{—}1_P9 (SEQ ID NO:591), a second amino acid sequence being at least 90% homologous to RASSGLPRNTVVLFVPQQEAWVVERMGRFHRILEPGLNILIPVLDRIRYVQSLKEIVINVPEQSAVTLDNVT LQIDGVLYLRIMDPYKASYGVEDPEYAVTQLAQTTMRSELGKLSLDKVFRERESLNASIVDAINQAADCW GIRCLRYEIKDIHVPPRVKESMQMQVEAERRKR corresponding to amino acids 13-187 of Q9P042 (SEQ ID NO:639), which also corresponds to amino acids 27-201 of D11853_PEA_{—}1_P9 (SEQ ID NO:591), a bridging amino acid A corresponding to amino acid 202 of D11853_PEA_{—}1_P9 (SEQ ID NO:591), a third amino acid sequence being at least 90% homologous to TVLESEGTRESAINVAEGKKQAQILASEAEKAEQINQA corresponding to amino acids 189-226 of Q9P042 (SEQ ID NO:639), which also corresponds to amino acids 203-240 of D11853_PEA_{—}1_P9 (SEQ ID NO:591), a fourth amino acid sequence being at least 70%, optionally at least 80%, preferably at least 85%, more preferably at least 90% and most preferably at least 95% homologous to a polypeptide having the sequence AGQERVEAEGGARHGPLKIGAGAGSLGYFDFMGQASSVPSL (SEQ ID NO:1538) corresponding to amino acids 241-281 of D11853_PEA_{—}1_P9 (SEQ ID NO:591), and a fifth amino acid sequence being at least 90% homologous to AGEASAVLAKAKAKAEAIRILAAALTQHNGDAAASLTVAEQYVSAFSKLAKDSNTILLPSNPGDVTSMVA QAMGVYGALTKAPVPGTPDSLSSGSSRDVQGTDASLDEELDRVKMS corresponding to amino acids 227-342 of Q9P042 (SEQ ID NO:639), which also corresponds to amino acids 282-397 of D11853_PEA_{—}1_P9 (SEQ ID NO:591), wherein said first amino acid sequence, second amino acid sequence, bridging amino acid, third amino acid sequence, fourth amino acid sequence and fifth amino acid sequence are contiguous and in a sequential order.

2. An isolated polypeptide encoding for a head of D11853_PEA_{—}1_P9 (SEQ ID NO:591), comprising a polypeptide being at least 70%, optionally at least about 80%, preferably at least about 85%, more preferably at least about 90% and most preferably at least about 95% homologous to the sequence MLARAARGTGALLLRGSLLASGRAPR (SEQ ID NO:1530) of D11853_PEA_{—}1_P9 (SEQ ID NO:591).

3. An isolated polypeptide encoding for an edge portion of D11853_PEA_{—}1_P9 (SEQ ID NO:591) comprising an amino acid sequence being at least 70%, optionally at least about 80%, preferably at least about 85%, more preferably at least about 90% and most preferably at least about 95% homologous to the sequence encoding for AGQERVEAEGGARHGPLKIGAGAGSLGYFDFMGQASSVPSL (SEQ ID NO:1538), corresponding to D11853_PEA_{—}1_P9 (SEQ ID NO:591).

Comparison Report Between D11853_PEA_{—}1_P9 (SEQ ID NO:591) and BAC85377 (SEQ ID NO:640):

1. An isolated chimeric polypeptide encoding for D11853_PEA_{—}1_P9 (SEQ ID NO:591), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRI corresponding to amino acids 1-109 of D11853_PEA_{—}1_P9 (SEQ ID NO:591), a second amino acid sequence being at least 90% homologous to MDPYKASYGVEDPEYAVTQLAQTTMRSELGKLSLDKVFRERESLNASIVDAINQAADCWGIRCLRYEIKD IHVPPRVKESMQMQVEAERRKRATVLESEGTRESAINVAEGKKQAQILASEAEKAEQINQA corresponding to amino acids 1-131 of BAC85377 (SEQ ID NO:640), which also corresponds to amino acids 110-240 of D11853_PEA_{—}1_P9 (SEQ ID NO:591), a third amino acid sequence being at least 70%, optionally at least 80%, preferably at least 85%, more preferably at least 90% and most preferably at least 95% homologous to a polypeptide having the sequence AGQERVEAEGGARHGPLKIGAGAGSLGYFDFMGQASSVPSL (SEQ ID NO:1538) corresponding to amino acids 241-281 of D11853_PEA_{—}1_P9 (SEQ ID NO:591), a fourth amino acid sequence being at least 90% homologous to AGEASAVLAKAKAKAEAIRILAAALTQH corresponding to amino acids 132-159 of BAC85377 (SEQ ID NO:640), which also corresponds to amino acids 282-309 of D11853_PEA_{—}1_P9 (SEQ ID NO:591), and a fifth amino acid sequence being at least 70%, optionally at least 80%, preferably at least 85%, more preferably at least 90% and most preferably at least 95% homologous to a polypeptide having the sequence NGDAAASLTVAEQYVSAFSKLAKDSNTILLPSNPGDVTSMVAQAMGVYGALTKAPVPGTPDSLSSGSSRD VQGTDASLDEELDRVKMS (SEQ ID NO:1531) corresponding to amino acids 310-397 of D11853_PEA_{—}1_P9 (SEQ ID NO:591), wherein said first amino acid sequence, second amino acid sequence, third amino acid sequence, fourth amino acid sequence and fifth amino acid sequence are contiguous and in a sequential order.

2. An isolated polypeptide encoding for a head of D11853_PEA_{—}1_P9 (SEQ ID NO:591), comprising a polypeptide being at least 70%, optionally at least about 80%, preferably at least about 85%, more preferably at least about 90% and most preferably at least about 95% homologous to the sequence MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRI of D11853_PEA_{—}1_P9 (SEQ ID NO:591).

3. An isolated polypeptide encoding for an edge portion of D11853_PEA_{—}1_P9 (SEQ ID NO:591), comprising an amino acid sequence being at least 70%, optionally at least about 80%, preferably at least about 85%, more preferably at least about 90% and most preferably at least about 95% homologous to the sequence encoding for AGQERVEAEGGARHGPLKIGAGAGSLGYFDFMGQASSVPSL (SEQ ID NO:1538), corresponding to D11853_PEA_{—}1_P9 (SEQ ID NO:591).

4. An isolated polypeptide encoding for a tail of D11853_PEA_{—}1_P9 (SEQ ID NO:591), comprising a polypeptide being at least 70%, optionally at least about 80%, preferably at least about 85%, more preferably at least about 90% and most preferably at least about 95% homologous to the sequence NGDAAASLTVAEQYVSAFSKLAKDSNTILLPSNPGDVTSMVAQAMGVYGALTKAPVPGTPDSLSSGSSRD VQGTDASLDEELDRVKMS (SEQ ID NO:1531) in D11853_PEA_{—}1_P9 (SEQ ID NO:591).

Comparison Report Between D11853_PEA_{—}1_P9 (SEQ ID NO:591) and Q96FY2 (SEQ ID NO:638):

1. An isolated chimeric polypeptide encoding for D11853_PEA_{—}1_P9 (SEQ ID NO:591), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQ corresponding to amino acids 1-128 of Q96FY2 (SEQ ID NO:638), which also corresponds to amino acids 1-128 of D11853_PEA_{—}1_P9 (SEQ ID NO:591), a bridging amino acid L corresponding to amino acid 129 of D11853_PEA_{—}1_P9 (SEQ ID NO:591), a second amino acid sequence being at least 90% homologous to AQTTMRSELGKLSLDKVFRERESLNASIVDAINQAADCWGIRCLRYEIKDIHVPPRVKESMQMQVEAERR KRATVLESEGTRESAINVAEGKKQAQILASEAEKAEQINQA corresponding to amino acids 130-240 of Q96FY2 (SEQ ID NO:638), which also corresponds to amino acids 130-240 of D11853_PEA_{—}1_P9 (SEQ ID NO:591), a third amino acid sequence being at least 70%, optionally at least 80%, preferably at least 85%, more preferably at least 90% and most preferably at least 95% homologous to a polypeptide having the sequence AGQERVEAEGGARHGPLKIGAGAGSLGYFDFMGQASSVPSL (SEQ ID NO:1538) corresponding to amino acids 241-281 of D11853_PEA_{—}1_P9 (SEQ ID NO:591), and a fourth amino acid sequence being at least 90% homologous to AGEASAVLAKAKAKAEAIRILAAALTQHNGDAAASLTVAEQYVSAFSKLAKDSNTILLPSNPGDVTSMVA QAMGVYGALTKAPVPGTPDSLSSGSSRDVQGTDASLDEELDRVKMS corresponding to amino acids 241-356 of Q96FY2 (SEQ ID NO:638), which also corresponds to amino acids 282-397 of D11853_PEA_{—}1_P9 (SEQ ID NO:591), wherein said first amino acid sequence, bridging amino acid, second amino acid sequence, third amino acid sequence and fourth amino acid sequence are contiguous and in a sequential order.

2. An isolated polypeptide encoding for an edge portion of D11853_PEA_{—}1_P9 (SEQ ID NO:591) comprising an amino acid sequence being at least 70%, optionally at least about 80%, preferably at least about 85%, more preferably at least about 90% and most preferably at least about 95% homologous to the sequence encoding for AGQERVEAEGGARHGPLKIGAGAGSLGYFDFMGQASSVPSL (SEQ ID NO:1538), corresponding to D11853_PEA_{—}1_P9 (SEQ ID NO:591).

Comparison Report Between D11853_PEA_{—}1_P9 (SEQ ID NO:591) and Q9UJZ1 (SEQ ID NO:637):

1. An isolated chimeric polypeptide encoding for D11853_PEA_{—}1_P9 (SEQ ID NO:591), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQLAQTTMRSELGKLS LDKVFRERESLNASIVDAINQAADCWGIRCLRYEIKDIHVPPRVKESMQMQVEAERRKRATVLESEGTRES AINVAEGKKQAQILASEAEKAEQINQA corresponding to amino acids 1-240 of Q9UJZ1 (SEQ ID NO:637), which also corresponds to amino acids 1-240 of D11853_PEA_{—}1_P9 (SEQ ID NO:591), a second amino acid sequence being at least 70%, optionally at least 80%, preferably at least 85%, more preferably at least 90% and most preferably at least 95% homologous to a polypeptide having the sequence AGQERVEAEGGARHGPLKIGAGAGSLGYFDFMGQASSVPSL (SEQ ID NO:1538) corresponding to amino acids 241-281 of D11853_PEA_{—}1_P9 (SEQ ID NO:591), and a third amino acid sequence being at least 90% homologous to AGEASAVLAKAKAKAEAIRILAAALTQHNGDAAASLTVAEQYVSAFSKLAKDSNTILLPSNPGDVTSMVA QAMGVYGALTKAPVPGTPDSLSSGSSRDVQGTDASLDEELDRVKMS corresponding to amino acids 241-356 of Q9UJZ1 (SEQ ID NO:637), which also corresponds to amino acids 282-397 of D11853_PEA_{—}1_P9 (SEQ ID NO:591), wherein said first amino acid sequence, second amino acid sequence and third amino acid sequence are contiguous and in a sequential order.

2. An isolated polypeptide encoding for an edge portion of D11853_PEA_{—}1_P9 (SEQ ID NO:591) comprising an amino acid sequence being at least 70%, optionally at least about 80%, preferably at least about 85%, more preferably at least about 90% and most preferably at least about 95% homologous to the sequence encoding for AGQERVEAEGGARHGPLKIGAGAGSLGYFDFMGQASSVPSL (SEQ ID NO:1538), corresponding to D11853_PEA_{—}1_P9 (SEQ ID NO:591).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P9 (SEQ ID NO:591) also has the following non-silent SNPs (Single Nucleotide Polymorphisms) as listed in Table 12, (given according to their position(s) on the amino acid sequence, with the alternative amino acid(s) listed; the last column indicates whether the SNP is known or not; the presence of known SNPs in variant protein D11853_PEA_{—}1_P9 (SEQ ID NO:591) sequence provides support for the deduced sequence of this variant protein according to the present invention).

TABLE 12
Amino acid mutations

| SNP position(s) on | Alternative | Previously |
|---|---|---|
| amino acid sequence | amino acid(s) | known SNP? |
| 25 | P -> | No |
| 32 | L -> | No |
| 178 | K -> | No |
| 178 | K -> R | No |
| 178 | K -> T | No |
| 185 | R -> G | No |
| 187 | K -> T | No |
| 206 | E -> K | No |
| 230 | E -> K | No |
| 230 | E -> Q | No |
| 235 | E -> | No |
| 235 | E -> D | No |
| 239 | Q -> P | No |
| 285 | A -> G | No |
| 285 | A -> | No |
| 308 | Q -> H | No |
| 319 | V -> G | No |
| 325 | S -> N | No |
| 325 | S -> T | No |
| 340 | P -> | No |
| 340 | P -> A | No |
| 367 | G -> | No |
| 370 | D -> N | No |
| 381 | Q -> | No |

Variant protein D11853_PEA_{—}1_P9 (SEQ ID NO:591) is encoded by the following transcript(s): D11853_PEA_{—}1_T13 (SEQ ID NO:63), for which the sequence(s) is/are given at the end of the application. The coding portion of transcript D11853_PEA_{—}1_T13 (SEQ ID NO:63) is shown in bold; this coding portion starts at position 108 and ends at position 1298. The transcript also has the following SNPs as listed in Table 13 (given according to their position on the nucleotide sequence, with the alternative nucleic acid listed; the last column indicates whether the SNP is known or not; the presence of known SNPs in variant protein D11853_PEA_{—}1_P9 (SEQ ID NO:591) sequence provides support for the deduced sequence of this variant protein according to the present invention).

TABLE 13
Nucleic acid SNPs

| SNP position on | Alternative | Previously |
|---|---|---|
| nucleotide sequence | nucleic acid | known SNP? |
| 180 | C -> | No |
| 182 | G -> | No |
| 185 | -> C | No |
| 201 | T -> | No |
| 640 | A -> C | No |
| 640 | A -> G | No |
| 641 | G -> | No |
| 641 | G -> A | No |
| 660 | C -> G | No |
| 667 | A -> C | No |
| 723 | G -> A | No |
| 795 | G -> A | No |
| 795 | G -> C | No |
| 812 | A -> | No |
| 812 | A -> C | No |
| 823 | A -> C | No |
| 961 | C -> | No |
| 961 | C -> G | No |
| 1031 | A -> C | No |
| 1055 | A -> C | No |
| 1063 | T -> G | No |
| 1081 | G -> A | No |
| 1081 | G -> C | No |
| 1089 | -> C | No |
| 1089 | -> T | No |
| 1096 | -> G | No |
| 1125 | C -> | No |
| 1125 | C -> G | No |
| 1156 | -> G | No |
| 1206 | G -> | No |
| 1215 | G -> A | No |
| 1235 | C -> T | No |
| 1250 | G -> | No |
| 1331 | G -> A | No |
| 1334 | A -> | No |
| 1334 | A -> C | No |
| 1380 | T -> | No |
| 1383 | T -> C | No |
| 1383 | T -> G | No |
| 1420 | T -> C | Yes |

Variant protein D11853_PEA_{—}1_P10 (SEQ ID NO:592) according to the present invention has an amino acid sequence as given at the end of the application; it is encoded by transcript(s) D11853_PEA_{—}1_T14 (SEQ ID NO:64). An alignment is given to the known protein (Membrane associated protein SLP-2 (SEQ ID NO:637)) at the end of the application. One or more alignments to one or more previously published protein sequences are given at the end of the application. A brief description of the relationship of the variant protein according to the present invention to each such aligned protein is as follows:

Comparison Report Between D11853_PEA_{—}1_P10 (SEQ ID NO:592) and Q9P042 (SEQ ID NO:639):

1. An isolated chimeric polypeptide encoding for D11853_PEA_{—}1_P10 (SEQ ID NO:592), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPR (SEQ ID NO:1530) corresponding to amino acids 1-26 of D11853_PEA_{—}1_P10 (SEQ ID NO:592), a second amino acid sequence being at least 90% homologous to RASSGLPRNTVVLFVPQQEAWVVERMGRFHRILEPGLNILIPVLDRIRYVQSLKEIVINVPEQSAVTLDNVT LQIDGVLYLRIMDPYKASYGVEDPEYAVTQLAQTTMRSELGKLSLDKVFRERESLNASIVDAINQAADCW GIRCLRYEIKDIHVPPRVKESMQMQVEAERRKR corresponding to amino acids 13-187 of Q9P042 (SEQ ID NO:639), which also corresponds to amino acids 27-201 of D11853_PEA_{—}1_P10 (SEQ ID NO:592), a bridging amino acid A corresponding to amino acid 202 of D11853_PEA_{—}1_P10 (SEQ ID NO:592), a third amino acid sequence being at least 90% homologous to TVLESEGTRESAINVAEGKKQAQILASEAEKAEQINQAAGEASAVLAKAKAKAEAIRILAAALTQH corresponding to amino acids 189-254 of Q9P042 (SEQ ID NO:639), which also corresponds to amino acids 269-313 of D11853_PEA_{—}1_P10 (SEQ ID NO:592), and a fourth amino acid sequence being at least 90% homologous to AMGVYGALTKAPVPGTPDSLSSGSSRDVQGTDASLDEELDRVKMS (SEQ ID NO:1540) corresponding to amino acids 298-342 of Q9P042 (SEQ ID NO:639), which also corresponds to amino acids 269-313 of D11853_PEA_{—}1_P10 (SEQ ID NO:592), wherein said first amino acid sequence, second amino acid sequence, bridging amino acid, third amino acid sequence and fourth amino acid sequence are contiguous and in a sequential order.

2. An isolated polypeptide encoding for a head of D11853_PEA_{—}1_P10 (SEQ ID NO:592), comprising a polypeptide being at least 70%, optionally at least about 80%, preferably at least about 85%, more preferably at least about 90% and most preferably at least about 95% homologous to the sequence MLARAARGTGALLLRGSLLASGRAPR (SEQ ID NO:1530) of D11853_PEA_{—}1_P10 (SEQ ID NO:592).

3. An isolated chimeric polypeptide encoding for an edge portion of D11853_PEA_{—}1_P10 (SEQ ID NO:592) comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise HA, having a structure as follows: a sequence starting from any of amino acid numbers 268−x to 268; and ending at any of amino acid numbers 269+((n−2)−x), in which x varies from 0 to n−2.

Comparison Report Between D11853_PEA_{—}1_P10 (SEQ ID NO:592) and BAC85377 (SEQ ID NO:640):

1. An isolated chimeric polypeptide encoding for D11853_PEA_{—}1_P10 (SEQ ID NO:592), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRI corresponding to amino acids 1-109 of D11853_PEA_{—}1_P10 (SEQ ID NO:592), a second amino acid sequence being at least 90% homologous to MDPYKASYGVEDPEYAVTQLAQTTMRSELGKLSLDKVFRERESLNASIVDAINQAADCWGIRCLRYEIKD IHVPPRVKESMQMQVEAERRKRATVLESEGTRESAINVAEGKKQAQILASEAEKAEQINQAAGEASAVLA KAKAKAEAIRILAAALTQH corresponding to amino acids 1-159 of BAC85377 (SEQ ID NO:640), which also corresponds to amino acids 110-268 of D11853_PEA_{—}1_P10 (SEQ ID NO:592), and a third amino acid sequence being at least 70%, optionally at least 80%, preferably at least 85%, more preferably at least 90% and most preferably at least 95% homologous to a polypeptide having the sequence AMGVYGALTKAPVPGTPDSLSSGSSRDVQGTDASLDEELDRVKMS (SEQ ID NO:1540) corresponding to amino acids 269-313 of D11853_PEA_{—}1_P10 (SEQ ID NO:592), wherein said first amino acid sequence, second amino acid sequence and third amino acid sequence are contiguous and in a sequential order.

2. An isolated polypeptide encoding for a head of D11853_PEA_{—}1_P10 (SEQ ID NO:592), comprising a polypeptide being at least 70%, optionally at least about 80%, preferably at least about 85%, more preferably at least about 90% and most preferably at least about 95% homologous to the sequence MLARAARGTGALLLRGSLLASGRAPRRASSGLPRNTVVLFVPQQEAWVVERMGRFHRILEPGLNILIPVLD RIRYVQSLKEWINVPEQSAVTLDNVTLQIDGVLYLRI of D11853_PEA_{—}1_P10 (SEQ ID NO:592).

3. An isolated polypeptide encoding for a tail of D11853_PEA_{—}1_P10 (SEQ ID NO:592), comprising a polypeptide being at least 70%, optionally at least about 80%, preferably at least about 85%, more preferably at least about 90% and most preferably at least about 95% homologous to the sequence AMGVYGALTKAPVPGTPDSLSSGSSRDVQGTDASLDEELDRVKMS (SEQ ID NO:1540) in D11853_PEA_{—}1_P10 (SEQ ID NO:592).

Comparison Report Between D11853_PEA_{—}1_P10 (SEQ ID NO:592) and Q96FY2 (SEQ ID NO:638):

1. An isolated chimeric polypeptide encoding for D11853_PEA_{—}1_P10 (SEQ ID NO:592), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQ corresponding to amino acids 1-128 of Q96FY2 (SEQ ID NO:638), which also corresponds to amino acids 1-128 of D11853_PEA_{—}1_P10 (SEQ ID NO:592), a bridging amino acid L corresponding to amino acid 129 of D11853_PEA_{—}1_P10 (SEQ ID NO:592), a second amino acid sequence being at least 90% homologous to AQTTMRSELGKLSLDKVFRERESLNASIVDAINQAADCWGIRCLRYEIKDIHVPPRVKESMQMQVEAERR KRATVLESEGTRESAINVAEGKKQAQILASEAEKAEQINQAAGEASAVLAKAKAKAEAIRILAAALTQH corresponding to amino acids 130-268 of Q96FY2 (SEQ ID NO:638), which also corresponds to amino acids 130-268 of D11853_PEA_{—}1_P10 (SEQ ID NO:592), and a third amino acid sequence being at least 90% homologous to AMGVYGALTKAPVPGTPDSLSSGSSRDVQGTDASLDEELDRVKMS (SEQ ID NO:1540) corresponding to amino acids 312-356 of Q96FY2 (SEQ ID NO:638), which also corresponds to amino acids 269-313 of D11853_PEA_{—}1_P10 (SEQ ID NO:592), wherein said first amino acid sequence, bridging amino acid, second amino acid sequence and third amino acid sequence are contiguous and in a sequential order.

2. An isolated chimeric polypeptide encoding for an edge portion of D11853_PEA_{—}1_P10 (SEQ ID NO:592) comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise HA, having a structure as follows: a sequence starting from any of amino acid numbers 268−x to 268; and ending at any of amino acid numbers 269+((n−2)−x), in which x varies from 0 to n−2.

Comparison Report Between D11853_PEA_{—}1_P10 (SEQ ID NO:592) and Q9UJZ1 (SEQ ID NO:637):

1. An isolated chimeric polypeptide encoding for D11853_PEA_{—}1_P10 (SEQ ID NO:592), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQLAQTTMRSELGKLS LDKVFRERESLNASIVDAINQAADCWGIRCLRYEIKDIHVPPRVKESMQMQVEAERRKRATVLESEGTRES AINVAEGKKQAQILASEAEKAEQINQAAGEASAVLAKAKAKAEAIRILAAALTQH corresponding to amino acids 1-268 of Q9UJZ1 (SEQ ID NO:637), which also corresponds to amino acids 1-268 of D11853_PEA_{—}1_P10 (SEQ ID NO:592), and a second amino acid sequence being at least 90% homologous to AMGVYGALTKAPVPGTPDSLSSGSSRDVQGTDASLDEELDRVKMS (SEQ ID NO:1540) corresponding to amino acids 312-356 of Q9UJZ1 (SEQ ID NO:637), which also corresponds to amino acids 269-313 of D11853_PEA_{—}1_P10 (SEQ ID NO:592), wherein said first amino acid sequence and second amino acid sequence are contiguous and in a sequential order.

2. An isolated chimeric polypeptide encoding for an edge portion of D11853_PEA_{—}1_P10 (SEQ ID NO:592) comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise HA, having a structure as follows: a sequence starting from any of amino acid numbers 268−x to 268; and ending at any of amino acid numbers 269+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P10 (SEQ ID NO:592) also has the following non-silent SNPs (Single Nucleotide Polymorphisms) as listed in Table 14, (given according to their position(s) on the amino acid sequence, with the alternative amino acid(s) listed; the last column indicates whether the SNP is known or not; the presence of known SNPs in variant protein D11853_PEA_{—}1_P10 (SEQ ID NO:592) sequence provides support for the deduced sequence of this variant protein according to the present invention).

TABLE 14
Amino acid mutations

| SNP position(s) on | Alternative | Previously |
|---|---|---|
| amino acid sequence | amino acid(s) | known SNP? |
| 25 | P -> | No |
| 32 | L -> | No |
| 178 | K -> | No |
| 178 | K -> R | No |
| 178 | K -> T | No |
| 185 | R -> G | No |
| 187 | K -> T | No |
| 206 | E -> K | No |
| 230 | E -> K | No |
| 230 | E -> Q | No |
| 235 | E -> | No |
| 235 | E -> D | No |
| 239 | Q -> P | No |
| 244 | A -> | No |
| 244 | A -> G | No |
| 267 | Q -> H | No |
| 283 | G -> | No |
| 286 | D -> N | No |
| 297 | Q -> | No |

Variant protein D11853_PEA_{—}1_P10 (SEQ ID NO:592) is encoded by the following transcript(s): D11853_PEA_{—}1_T14 (SEQ ID NO:64), for which the sequence(s) is/are given at the end of the application. The coding portion of transcript D11853_PEA_{—}1_T14 (SEQ ID NO:64) is shown in bold; this coding portion starts at position 108 and ends at position 1046. The transcript also has the following SNPs as listed in Table 15 (given according to their position on the nucleotide sequence, with the alternative nucleic acid listed; the last column indicates whether the SNP is known or not; the presence of known SNPs in variant protein D11853_PEA_{—}1_P10 (SEQ ID NO:592) sequence provides support for the deduced sequence of this variant protein according to the present invention).

TABLE 15
Nucleic acid SNPs

| SNP position on | Alternative | Previously |
|---|---|---|
| nucleotide sequence | nucleic acid | known SNP? |
| 180 | C -> | No |
| 182 | G -> | No |
| 185 | -> C | No |
| 201 | T -> | No |
| 640 | A -> C | No |
| 640 | A -> G | No |
| 641 | G -> | No |
| 641 | G -> A | No |
| 660 | C -> G | No |
| 667 | A -> C | No |
| 723 | G -> A | No |
| 795 | G -> A | No |
| 795 | G -> C | No |
| 812 | A -> | No |
| 812 | A -> C | No |
| 823 | A -> C | No |
| 838 | C -> | No |
| 838 | C -> G | No |
| 908 | A -> C | No |
| 954 | G -> | No |
| 963 | G -> A | No |
| 983 | C -> T | No |
| 998 | G -> | No |
| 1079 | G -> A | No |
| 1082 | A -> | No |
| 1082 | A -> C | No |
| 1128 | T -> | No |
| 1131 | T -> C | No |
| 1131 | T -> G | No |
| 1168 | T -> C | Yes |

Variant protein D11853_PEA_{—}1_P11 (SEQ ID NO:593) according to the present invention has an amino acid sequence as given at the end of the application; it is encoded by transcript(s) D11853_PEA_{—}1_T15 (SEQ ID NO:65). An alignment is given to the known protein (Membrane associated protein SLP-2 (SEQ ID NO:637)) at the end of the application. One or more alignments to one or more previously published protein sequences are given at the end of the application. A brief description of the relationship of the variant protein according to the present invention to each such aligned protein is as follows:

Comparison Report Between D11853_PEA_{—}1_P11 (SEQ ID NO:593) and Q9P042 (SEQ ID NO:639):

1. An isolated chimeric polypeptide encoding for D11853_PEA_{—}1_P11 (SEQ ID NO:593), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPR (SEQ ID NO:1530) corresponding to amino acids 1-26 of D11853_PEA_{—}1_P11 (SEQ ID NO:593), a second amino acid sequence being at least 90% homologous to RASSGLPRNTVVLFVPQQEAWVVERMGRFHRILEPGLNILIPVLDRIRYVQSLKEIVINVPEQSAVTLDNVT LQIDGVLYLRIMDPYKASYGVEDPEYAVTQLAQTTMRSELGKLSLDKVFRERESLNASIVDAINQAADCW GIRCLRYEIKDIHVPPRVKESMQMQVEAERRKR corresponding to amino acids 13-187 of Q9P042 (SEQ ID NO:639), which also corresponds to amino acids 27-201 of D11853_PEA_{—}1_P11 (SEQ ID NO:593), a bridging amino acid A corresponding to amino acid 202 of D11853_PEA_{—}1_P11 (SEQ ID NO:593), a third amino acid sequence being at least 90% homologous to TVLESEGTRESAINVAEGKKQAQILASEAEKAEQINQA corresponding to amino acids 189-226 of Q9P042 (SEQ ID NO:639), which also corresponds to amino acids 203-240 of D11853_PEA_{—}1_P11 (SEQ ID NO:593), a fourth amino acid sequence being at least 70%, optionally at least 80%, preferably at least 85%, more preferably at least 90% and most preferably at least 95% homologous to a polypeptide having the sequence AGQERVEAEGGARHGPLKIGAGAGSLGYFDFMGQASSVPSL (SEQ ID NO:1538) corresponding to amino acids 241-281 of D11853_PEA_{—}1_P11 (SEQ ID NO:593), a fifth amino acid sequence being at least 90% homologous to AGEASAVLAKAKAKAEAIRILAAALTQH corresponding to amino acids 227-254 of Q9P042 (SEQ ID NO:639), which also corresponds to amino acids 282-309 of D11853_PEA_{—}1_P11 (SEQ ID NO:593), and a sixth amino acid sequence being at least 70%, optionally at least 80%, preferably at least 85%, more preferably at least 90% and most preferably at least 95% homologous to a polypeptide having the sequence VRGPWVGMGTGIDSGRGSLIYA (SEQ ID NO:1535) corresponding to amino acids 310-331 of D11853_PEA_{—}1_P11 (SEQ ID NO:593), wherein said first amino acid sequence, second amino acid sequence, bridging amino acid, third amino acid sequence, fourth amino acid sequence, fifth amino acid sequence and sixth amino acid sequence are contiguous and in a sequential order.

2. An isolated polypeptide encoding for a head of D11853_PEA_{—}1_P11 (SEQ ID NO:593), comprising a polypeptide being at least 70%, optionally at least about 80%, preferably at least about 85%, more preferably at least about 90% and most preferably at least about 95% homologous to the sequence MLARAARGTGALLLRGSLLASGRAPR (SEQ ID NO:1530) of D11853_PEA_{—}1_P11 (SEQ ID NO:593).

3. An isolated polypeptide encoding for an edge portion of D11853_PEA_{—}1_P11 (SEQ ID NO:593) comprising an amino acid sequence being at least 70%, optionally at least about 80%, preferably at least about 85%, more preferably at least about 90% and most preferably at least about 95% homologous to the sequence encoding for AGQERVEAEGGARHGPLKIGAGAGSLGYFDFMGQASSVPSL (SEQ ID NO:1538), corresponding to D11853_PEA_{—}1_P11 (SEQ ID NO:593).

4. An isolated polypeptide encoding for a tail of D11853_PEA_{—}1_P11 (SEQ ID NO:593), comprising a polypeptide being at least 70%, optionally at least about 80%, preferably at least about 85%, more preferably at least about 90% and most preferably at least about 95% homologous to the sequence VRGPWVGMGTGIDSGRGSLIYA (SEQ ID NO:1535) in D11853_PEA_{—}1_P11 (SEQ ID NO:593).

Comparison Report Between D11853_PEA_{—}1_P11 (SEQ ID NO:593) and BAC85377 (SEQ ID NO:640):

1. An isolated chimeric polypeptide encoding for D11853_PEA_{—}1_P11 (SEQ ID NO:593), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRI corresponding to amino acids 1-109 of D11853_PEA_{—}1_P11 (SEQ ID NO:593), a second amino acid sequence being at least 90% homologous to MDPYKASYGVEDPEYAVTQLAQTTMRSELGKLSLDKVFRERESLNASIVDAINQAADCWGIRCLRYEIKD IHVPPRVKESMQMQVEAERRKRATVLESEGTRESAINVAEGKKQAQILASEAEKAEQINQA corresponding to amino acids 1-131 of BAC85377 (SEQ ID NO:640), which also corresponds to amino acids 110-240 of D11853_PEA_{—}1_P11 (SEQ ID NO:593), a third amino acid sequence being at least 70%, optionally at least 80%, preferably at least 85%, more preferably at least 90% and most preferably at least 95% homologous to a polypeptide having the sequence AGQERVEAEGGARHGPLKIGAGAGSLGYFDFMGQASSVPSL (SEQ ID NO:1538) corresponding to amino acids 241-281 of D11853_PEA_{—}1_P11 (SEQ ID NO:593), and a fourth amino acid sequence being at least 90% homologous to AGEASAVLAKAKAKAEAIRILAAALTQHVRGPWVGMGTGIDSGRGSLIYA corresponding to amino acids 132-181 of BAC85377 (SEQ ID NO:640), which also corresponds to amino acids 282-331 of D11853_PEA_{—}1_P11 (SEQ ID NO:593), wherein said first amino acid sequence, second amino acid sequence, third amino acid sequence and fourth amino acid sequence are contiguous and in a sequential order.

2. An isolated polypeptide encoding for a head of D11853_PEA_{—}1_P11 (SEQ ID NO:593), comprising a polypeptide being at least 70%, optionally at least about 80%, preferably at least about 85%, more preferably at least about 90% and most preferably at least about 95% homologous to the sequence MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRI of D11853_PEA_{—}1_P11 (SEQ ID NO:593).

3. An isolated polypeptide encoding for an edge portion of D11853_PEA_{—}1_P11 (SEQ ID NO:593) comprising an amino acid sequence being at least 70%, optionally at least about 80%, preferably at least about 85%, more preferably at least about 90% and most preferably at least about 95% homologous to the sequence encoding for AGQERVEAEGGARHGPLKIGAGAGSLGYFDFMGQASSVPSL (SEQ ID NO:1538), corresponding to D11853_PEA_{—}1_P11 (SEQ ID NO:593).

Comparison Report Between D11853_PEA_{—}1_P11 (SEQ ID NO:593) and Q96FY2 (SEQ ID NO:638):

1. An isolated chimeric polypeptide encoding for D11853_PEA_{—}1_P11 (SEQ ID NO:593), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQ corresponding to amino acids 1-128 of Q96FY2 (SEQ ID NO:638), which also corresponds to amino acids 1-128 of D11853_PEA_{—}1_P11 (SEQ ID NO:593), a bridging amino acid L corresponding to amino acid 129 of D11853_PEA_{—}1_P11 (SEQ ID NO:593), a second amino acid sequence being at least 90% homologous to AQTTMRSELGKLSLDKVFRERESLNASIVDAINQAADCWGIRCLRYEIKDIHVPPRVKESMQMQVEAERR KRATVLESEGTRESAINVAEGKKQAQILASEAEKAEQINQA corresponding to amino acids 130-240 of Q96FY2 (SEQ ID NO:638), which also corresponds to amino acids 130-240 of D11853_PEA_{—}1_P11 (SEQ ID NO:593), a third amino acid sequence being at least 70%, optionally at least 80%, preferably at least 85%, more preferably at least 90% and most preferably at least 95% homologous to a polypeptide having the sequence AGQERVEAEGGARHGPLKIGAGAGSLGYFDFMGQASSVPSL (SEQ ID NO:1538) corresponding to amino acids 241-281 of D11853_PEA_{—}1_P11 (SEQ ID NO:593), a fourth amino acid sequence being at least 90% homologous to AGEASAVLAKAKAKAEAIRILAAALTQH corresponding to amino acids 241-268 of Q96FY2 (SEQ ID NO:638), which also corresponds to amino acids 282-309 of D11853_PEA_{—}1_P11 (SEQ ID NO:593), and a fifth amino acid sequence being at least 70%, optionally at least 80%, preferably at least 85%, more preferably at least 90% and most preferably at least 95% homologous to a polypeptide having the sequence VRGPWVGMGTGIDSGRGSLIYA (SEQ ID NO:1535) corresponding to amino acids 310-331 of D11853_PEA_{—}1_P11 (SEQ ID NO:593), wherein said first amino acid sequence, bridging amino acid, second amino acid sequence, third amino acid sequence, fourth amino acid sequence and fifth amino acid sequence are contiguous and in a sequential order.

2. An isolated polypeptide encoding for an edge portion of D11853_PEA_{—}1_P11 (SEQ ID NO:593), comprising an amino acid sequence being at least 70%, optionally at least about 80%, preferably at least about 85%, more preferably at least about 90% and most preferably at least about 95% homologous to the sequence encoding for AGQERVEAEGGARHGPLKIGAGAGSLGYFDFMGQASSVPSL (SEQ ID NO:1538), corresponding to D11853_PEA_{—}1_P11 (SEQ ID NO:593).

3. An isolated polypeptide encoding for a tail of D11853_PEA_{—}1_P11 (SEQ ID NO:593), comprising a polypeptide being at least 70%, optionally at least about 80%, preferably at least about 85%, more preferably at least about 90% and most preferably at least about 95% homologous to the sequence VRGPWVGMGTGIDSGRGSLIYA (SEQ ID NO:1535) in D11853_PEA_{—}1_P11 (SEQ ID NO:593).

Comparison Report Between D11853_PEA_{—}1_P11 (SEQ ID NO:593) and Q9UJZ1 (SEQ ID NO:637):

1. An isolated chimeric polypeptide encoding for D11853_PEA_{—}1_P11 (SEQ ID NO:593), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQLAQTTMRSELGKLS LDKVFRERESLNASIVDAINQAADCWGIRCLRYEIKDIHVPPRVKESMQMQVEAERRKRATVLESEGTRES AINVAEGKKQAQILASEAEKAEQINQA corresponding to amino acids 1-240 of Q9UJZ1 (SEQ ID NO:637), which also corresponds to amino acids 1-240 of D11853_PEA_{—}1_P11 (SEQ ID NO:593), a second amino acid sequence being at least 70%, optionally at least 80%, preferably at least 85%, more preferably at least 90% and most preferably at least 95% homologous to a polypeptide having the sequence AGQERVEAEGGARHGPLKIGAGAGSLGYFDFMGQASSVPSL (SEQ ID NO:1538) corresponding to amino acids 241-281 of D11853_PEA_{—}1_P11 (SEQ ID NO:593), a third amino acid sequence being at least 90% homologous to AGEASAVLAKAKAKAEAIRILAAALTQH corresponding to amino acids 241-268 of Q9UJZ1 (SEQ ID NO:637), which also corresponds to amino acids 282-309 of D11853_PEA_{—}1_P11 (SEQ ID NO:593), and a fourth amino acid sequence being at least 70%, optionally at least 80%, preferably at least 85%, more preferably at least 90% and most preferably at least 95% homologous to a polypeptide having the sequence VRGPWVGMGTGIDSGRGSLIYA (SEQ ID NO:1535) corresponding to amino acids 310-331 of D11853_PEA_{—}1_P11 (SEQ ID NO:593), wherein said first amino acid sequence, second amino acid sequence, third amino acid sequence and fourth amino acid sequence are contiguous and in a sequential order.

2. An isolated polypeptide encoding for an edge portion of D11853_PEA_{—}1_P11 (SEQ ID NO:593) comprising an amino acid sequence being at least 70%, optionally at least about 80%, preferably at least about 85%, more preferably at least about 90% and most preferably at least about 95% homologous to the sequence encoding for AGQERVEAEGGARHGPLKIGAGAGSLGYFDFMGQASSVPSL (SEQ ID NO:1538), corresponding to D11853_PEA_{—}1_P11 (SEQ ID NO:593).

3. An isolated polypeptide encoding for a tail of D11853_PEA_{—}1_P11 (SEQ ID NO:593), comprising a polypeptide being at least 70%, optionally at least about 80%, preferably at least about 85%, more preferably at least about 90% and most preferably at least about 95% homologous to the sequence VRGPWVGMGTGIDSGRGSLIYA (SEQ ID NO:1535) in D11853_PEA_{—}1_P11 (SEQ ID NO:593).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P11 (SEQ ID NO:593) also has the following non-silent SNPs (Single Nucleotide Polymorphisms) as listed in Table 16, (given according to their position(s) on the amino acid sequence, with the alternative amino acid(s) listed; the last column indicates whether the SNP is known or not; the presence of known SNPs in variant protein D11853_PEA_{—}1_P11 (SEQ ID NO:593) sequence provides support for the deduced sequence of this variant protein according to the present invention).

TABLE 16
Amino acid mutations

| SNP position(s) on | Alternative | Previously |
|---|---|---|
| amino acid sequence | amino acid(s) | known SNP? |
| 25 | P -> | No |
| 32 | L -> | No |
| 178 | K -> | No |
| 178 | K -> R | No |
| 178 | K -> T | No |
| 185 | R -> G | No |
| 187 | K -> T | No |
| 206 | E -> K | No |
| 230 | E -> K | No |
| 230 | E -> Q | No |
| 235 | E -> | No |
| 235 | E -> D | No |
| 239 | Q -> P | No |
| 285 | A -> | No |
| 285 | A -> G | No |
| 308 | Q -> H | No |

Variant protein D11853_PEA_{—}1_P11 (SEQ ID NO:593) is encoded by the following transcript(s): D11853_PEA_{—}1_T15 (SEQ ID NO:65), for which the sequence(s) is/are given at the end of the application. The coding portion of transcript D11853_PEA_{—}1_T15 (SEQ ID NO:65) is shown in bold; this coding portion starts at position 108 and ends at position 1100. The transcript also has the following SNPs as listed in Table 17 (given according to their position on the nucleotide sequence, with the alternative nucleic acid listed; the last column indicates whether the SNP is known or not; the presence of known SNPs in variant protein D11853_PEA_{—}1_P11 (SEQ ID NO:593) sequence provides support for the deduced sequence of this variant protein according to the present invention).

TABLE 17
Nucleic acid SNPs

| SNP position on | Alternative | Previously |
|---|---|---|
| nucleotide seqence | nucleic acid | known SNP? |
| 180 | C -> | No |
| 182 | G -> | No |
| 185 | -> C | No |
| 201 | T -> | No |
| 640 | A -> C | No |
| 640 | A -> G | No |
| 641 | G -> | No |
| 641 | G -> A | No |
| 660 | C -> G | No |
| 667 | A -> C | No |
| 723 | G -> A | No |
| 795 | G -> A | No |
| 795 | G -> C | No |
| 812 | A -> | No |
| 812 | A -> C | No |
| 823 | A -> C | No |
| 961 | C -> | No |
| 961 | C -> G | No |
| 1031 | A -> C | No |
| 1260 | A -> C | No |
| 1268 | T -> G | No |
| 1286 | G -> A | No |
| 1286 | G -> C | No |
| 1294 | -> C | No |
| 1294 | -> T | No |
| 1301 | -> G | No |
| 1330 | C -> | No |
| 1330 | C -> G | No |
| 1361 | -> G | No |
| 1411 | G -> | No |
| 1420 | G -> A | No |
| 1440 | C -> T | No |
| 1455 | G -> | No |
| 1536 | G -> A | No |
| 1539 | A -> | No |
| 1539 | A -> C | No |
| 1585 | T -> | No |
| 1588 | T -> C | No |
| 1588 | T -> G | No |
| 1625 | T -> C | Yes |

Variant protein D11853_PEA_{—}1_P12 (SEQ ID NO:594) according to the present invention has an amino acid sequence as given at the end of the application; it is encoded by transcript(s) D11853_PEA_{—}1_T16 (SEQ ID NO:66). An alignment is given to the known protein (Membrane associated protein SLP-2 (SEQ ID NO:637)) at the end of the application. One or more alignments to one or more previously published protein sequences are given at the end of the application. A brief description of the relationship of the variant protein according to the present invention to each such aligned protein is as follows:

Comparison Report Between D11853_PEA_{—}1_P12 (SEQ ID NO:594) and Q9P042 (SEQ ID NO:639):

1. An isolated chimeric polypeptide encoding for D11853_PEA_{—}1_P12 (SEQ ID NO:594), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPR (SEQ ID NO:1530) corresponding to amino acids 1-26 of D11853_PEA_{—}1_P12 (SEQ ID NO:594), a second amino acid sequence being at least 90% homologous to RASSGLPRNTVVLFVPQQEAWVVERMGRFHRILEPGLNILIPVLDRIRYVQSLKEIVINVPEQSAVTLDNVT LQIDGVLYLRIMDPYKASYGVEDPEYAVTQLAQTTMRSELGKLSLDKVFR corresponding to amino acids 13-134 of Q9P042 (SEQ ID NO:639), which also corresponds to amino acids 27-148 of D11853_PEA_{—}1_P12 (SEQ ID NO:594), and a third amino acid sequence being at least 70%, optionally at least 80%, preferably at least 85%, more preferably at least 90% and most preferably at least 95% homologous to a polypeptide having the sequence VSRSEPELGFEDTNLTLLIFSEGQDQSQALLSVGP (SEQ ID NO:1545) corresponding to amino acids 149-183 of D11853_PEA_{—}1_P12 (SEQ ID NO:594), wherein said first amino acid sequence, second amino acid sequence and third amino acid sequence are contiguous and in a sequential order.

2. An isolated polypeptide encoding for a head of D11853_PEA_{—}1_P12 (SEQ ID NO:594), comprising a polypeptide being at least 70%, optionally at least about 80%, preferably at least about 85%, more preferably at least about 90% and most preferably at least about 95% homologous to the sequence MLARAARGTGALLLRGSLLASGRAPR (SEQ ID NO:1530) of D11853_PEA_{—}1_P12 (SEQ ID NO:594).

3. An isolated polypeptide encoding for a tail of D11853_PEA_{—}1_P12 (SEQ ID NO:594), comprising a polypeptide being at least 70%, optionally at least about 80%, preferably at least about 85%, more preferably at least about 90% and most preferably at least about 95% homologous to the sequence VSRSEPELGFEDTNLTLLIFSEGQDQSQALLSVGP (SEQ ID NO:1545) in D11853_PEA_{—}1_P12 (SEQ ID NO:594).

Comparison Report Between D11853_PEA_{—}1_P12 (SEQ ID NO:594) and Q96FY2 (SEQ ID NO:638):

1. An isolated chimeric polypeptide encoding for D11853_PEA_{—}1_P12 (SEQ ID NO:594), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQ corresponding to amino acids 1-128 of Q96FY2 (SEQ ID NO:638), which also corresponds to amino acids 1-128 of D11853_PEA_{—}1_P12 (SEQ ID NO:594), a bridging amino acid L corresponding to amino acid 129 of D11853_PEA_{—}1_P12 (SEQ ID NO:594), a second amino acid sequence being at least 90% homologous to AQTTMRSELGKLSLDKVFR corresponding to amino acids 130-148 of Q96FY2 (SEQ ID NO:638), which also corresponds to amino acids 130-148 of D11853_PEA_{—}1_P12 (SEQ ID NO:594), and a third amino acid sequence being at least 70%, optionally at least 80%, preferably at least 85%, more preferably at least 90% and most preferably at least 95% homologous to a polypeptide having the sequence VSRSEPELGFEDTNLTLLIFSEGQDQSQALLSVGP (SEQ ID NO:1545) corresponding to amino acids 149-183 of D11853_PEA_{—}1_P12 (SEQ ID NO:594), wherein said first amino acid sequence, bridging amino acid, second amino acid sequence and third amino acid sequence are contiguous and in a sequential order.

2. An isolated polypeptide encoding for a tail of D11853_PEA_{—}1_P12 (SEQ ID NO:594), comprising a polypeptide being at least 70%, optionally at least about 80%, preferably at least about 85%, more preferably at least about 90% and most preferably at least about 95% homologous to the sequence VSRSEPELGFEDTNLTLLIFSEGQDQSQALLSVGP (SEQ ID NO:1545) in D11853_PEA_{—}1_P12 (SEQ ID NO:594).

Comparison Report Between D11853_PEA_{—}1_P12 (SEQ ID NO:594) and Q9UJZ1 (SEQ ID NO:637):

1. An isolated chimeric polypeptide encoding for D11853_PEA_{—}1_P12 (SEQ ID NO:594), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQLAQTTMRSELGKLS LDKVFR corresponding to amino acids 1-148 of Q9UJZ1 (SEQ ID NO:637), which also corresponds to amino acids 1-148 of D11853_PEA_{—}1_P12 (SEQ ID NO:594), and a second amino acid sequence being at least 70%, optionally at least 80%, preferably at least 85%, more preferably at least 90% and most preferably at least 95% homologous to a polypeptide having the sequence VSRSEPELGFEDTNLTLLIFSEGQDQSQALLSVGP (SEQ ID NO:1545) corresponding to amino acids 149-183 of D11853_PEA_{—}1_P12 (SEQ ID NO:594), wherein said first amino acid sequence and second amino acid sequence are contiguous and in a sequential order.

2. An isolated polypeptide encoding for a tail of D11853_PEA_{—}1_P12 (SEQ ID NO:594), comprising a polypeptide being at least 70%, optionally at least about 80%, preferably at least about 85%, more preferably at least about 90% and most preferably at least about 95% homologous to the sequence VSRSEPELGFEDTNLTLLIFSEGQDQSQALLSVGP (SEQ ID NO:1545) in D11853_PEA_{—}1_P12 (SEQ ID NO:594).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P12 (SEQ ID NO:594) is encoded by the following transcript(s): D11853_PEA_{—}1_T16 (SEQ ID NO:66), for which the sequence(s) is/are given at the end of the application. The coding portion of transcript D11853_PEA_{—}1_T16 (SEQ ID NO:66) is shown in bold; this coding portion starts at position 108 and ends at position 656. The transcript also has the following SNPs as listed in Table 18 (given according to their position on the nucleotide sequence, with the alternative nucleic acid listed; the last column indicates whether the SNP is known or not; the presence of known SNPs in variant protein D11853_PEA_{—}1_P12 (SEQ ID NO:594) sequence provides support for the deduced sequence of this variant protein according to the present invention).

TABLE 18
Nucleic acid SNPs

| SNP position on | Alternative | Previously |
|---|---|---|
| nucleotide seqence | nucleic acid | known SNP? |
| 180 | C -> | No |
| 182 | G -> | No |
| 185 | -> C | No |
| 201 | T -> | No |
| 789 | A -> C | No |
| 789 | A -> G | No |
| 790 | G -> | No |
| 790 | G -> A | No |
| 809 | C -> G | No |
| 816 | A -> C | No |
| 872 | G -> A | No |
| 944 | G -> A | No |
| 944 | G -> C | No |
| 961 | A -> | No |
| 961 | A -> C | No |
| 972 | A -> C | No |
| 987 | C -> | No |
| 987 | C -> G | No |
| 1057 | A -> C | No |
| 1081 | A -> C | No |
| 1089 | T -> G | No |
| 1107 | G -> A | No |
| 1107 | G -> C | No |
| 1115 | -> C | No |
| 1115 | -> T | No |
| 1122 | -> G | No |
| 1151 | C -> | No |
| 1151 | C -> G | No |
| 1182 | -> G | No |
| 1232 | G -> | No |
| 1241 | G -> A | No |
| 1261 | C -> T | No |
| 1276 | G -> | No |
| 1357 | G -> A | No |
| 1360 | A -> | No |
| 1360 | A -> C | No |
| 1406 | T -> | No |
| 1409 | T -> C | No |
| 1409 | T -> G | No |
| 1446 | T -> C | Yes |

Variant protein D11853_PEA_{—}1_P14 (SEQ ID NO:595) according to the present invention has an amino acid sequence as given at the end of the application; it is encoded by transcript(s) D11853_PEA_{—}1_T19 (SEQ ID NO:68). An alignment is given to the known protein (Membrane associated protein SLP-2 (SEQ ID NO:637)) at the end of the application. One or more alignments to one or more previously published protein sequences are given at the end of the application. A brief description of the relationship of the variant protein according to the present invention to each such aligned protein is as follows:

Comparison Report Between D11853_PEA_{—}1_P14 (SEQ ID NO:595) and Q9P042 (SEQ ID NO:639):

1. An isolated chimeric polypeptide encoding for D11853_PEA_{—}1_P14 (SEQ ID NO:595), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPR (SEQ ID NO:1530) corresponding to amino acids 1-26 of D11853_PEA_{—}1_P14 (SEQ ID NO:595), a second amino acid sequence being at least 90% homologous to RASSGLPRNTVVLFVPQQEAWVVERMGRFHRILEPGLNILIPVLDRIRYVQSLKEIVINVPEQSAVTLDNVT LQIDGVLYLRIMDPYKASYGVEDPEYAVTQLAQTTMRSELGKLSLDKVFRERESLNASIVDAINQAADCW GIRCLRYEIKDIHVPPRVKESMQMQV corresponding to amino acids 13-180 of Q9P042 (SEQ ID NO:639), which also corresponds to amino acids 27-194 of D11853_PEA_{—}1_P14 (SEQ ID NO:595), and a third amino acid sequence being at least 70%, optionally at least 80%, preferably at least 85%, more preferably at least 90% and most preferably at least 95% homologous to a polypeptide having the sequence GAKEGWEKGLRAPVPGGSRLPSCYDG (SEQ ID NO:1547) corresponding to amino acids 195-220 of D11853_PEA_{—}1_P14 (SEQ ID NO:595), wherein said first amino acid sequence, second amino acid sequence and third amino acid sequence are contiguous and in a sequential order.

2. An isolated polypeptide encoding for a head of D11853_PEA_{—}1_P14 (SEQ ID NO:595), comprising a polypeptide being at least 70%, optionally at least about 80%, preferably at least about 85%, more preferably at least about 90% and most preferably at least about 95% homologous to the sequence MLARAARGTGALLLRGSLLASGRAPR (SEQ ID NO:1530) of D11853_PEA_{—}1_P14 (SEQ ID NO:595).

3. An isolated polypeptide encoding for a tail of D11853_PEA_{—}1_P14 (SEQ ID NO:595), comprising a polypeptide being at least 70%, optionally at least about 80%, preferably at least about 85%, more preferably at least about 90% and most preferably at least about 95% homologous to the sequence GAKEGWEKGLRAPVPGGSRLPSCYDG (SEQ ID NO:1547) in D11853_PEA_{—}1_P14 (SEQ ID NO:595).

Comparison Report Between D11853_PEA_{—}1_P14 (SEQ ID NO:595) and Q96FY2 (SEQ ID NO:638):

1. An isolated chimeric polypeptide encoding for D11853_PEA_{—}1_P14 (SEQ ID NO:595), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQ corresponding to amino acids 1-128 of Q96FY2 (SEQ ID NO:638), which also corresponds to amino acids 1-128 of D11853_PEA_{—}1_P14 (SEQ ID NO:595), a bridging amino acid L corresponding to amino acid 129 of D11853_PEA_{—}1_P14 (SEQ ID NO:595), a second amino acid sequence being at least 90% homologous to AQTTMRSELGKLSLDKVFRERESLNASIVDAINQAADCWGIRCLRYEIKDIHVPPRVKESMQMQV corresponding to amino acids 130-194 of Q96FY2 (SEQ ID NO:638), which also corresponds to amino acids 130-194 of D11853_PEA_{—}1_P14 (SEQ ID NO:595), and a third amino acid sequence being at least 70%, optionally at least 80%, preferably at least 85%, more preferably at least 90% and most preferably at least 95% homologous to a polypeptide having the sequence GAKEGWEKGLRAPVPGGSRLPSCYDG (SEQ ID NO:1547) corresponding to amino acids 195-220 of D11853_PEA_{—}1_P14 (SEQ ID NO:595), wherein said first amino acid sequence, bridging amino acid, second amino acid sequence and third amino acid sequence are contiguous and in a sequential order.

2. An isolated polypeptide encoding for a tail of D11853_PEA_{—}1_P14 (SEQ ID NO:595), comprising a polypeptide being at least 70%, optionally at least about 80%, preferably at least about 85%, more preferably at least about 90% and most preferably at least about 95% homologous to the sequence GAKEGWEKGLRAPVPGGSRLPSCYDG (SEQ ID NO:1547) in D11853_PEA_{—}1_P14 (SEQ ID NO:595).

Comparison Report Between D11853_PEA_{—}1_P14 (SEQ ID NO:595) and Q9UJZ1 (SEQ ID NO:637):

1. An isolated chimeric polypeptide encoding for D11853_PEA_{—}1_P14 (SEQ ID NO:595), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQLAQTTMRSELGKLS LDKVFRERESLNASIVDAINQAADCWGIRCLRYEIKDIHVPPRVKESMQMQV corresponding to amino acids 1-194 of Q9UJZ1 (SEQ ID NO:637), which also corresponds to amino acids 1-194 of D11853_PEA_{—}1_P14 (SEQ ID NO:595), and a second amino acid sequence being at least 70%, optionally at least 80%, preferably at least 85%, more preferably at least 90% and most preferably at least 95% homologous to a polypeptide having the sequence GAKEGWEKGLRAPVPGGSRLPSCYDG (SEQ ID NO:1547) corresponding to amino acids 195-220 of D11853_PEA_{—}1_P14 (SEQ ID NO:595), wherein said first amino acid sequence and second amino acid sequence are contiguous and in a sequential order.

2. An isolated polypeptide encoding for a tail of D11853_PEA_{—}1_P14 (SEQ ID NO:595), comprising a polypeptide being at least 70%, optionally at least about 80%, preferably at least about 85%, more preferably at least about 90% and most preferably at least about 95% homologous to the sequence GAKEGWEKGLRAPVPGGSRLPSCYDG (SEQ ID NO:1547) in D11853_PEA_{—}1_P14 (SEQ ID NO:595).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P14 (SEQ ID NO:595) also has the following non-silent SNPs (Single Nucleotide Polymorphisms) as listed in Table 19, (given according to their position(s) on the amino acid sequence, with the alternative amino acid(s) listed; the last column indicates whether the SNP is known or not; the presence of known SNPs in variant protein D11853_PEA_{—}1_P14 (SEQ ID NO:595) sequence provides support for the deduced sequence of this variant protein according to the present invention).

TABLE 19
Amino acid mutations

| SNP position(s) on | Alternative | Previously |
|---|---|---|
| amino acid sequence | amino acid(s) | known SNP? |
| 25 | P -> | No |
| 32 | L -> | No |
| 178 | K -> | No |
| 178 | K -> R | No |
| 178 | K -> T | No |
| 185 | R -> G | No |
| 187 | K -> T | No |

Variant protein D11853_PEA_{—}1_P14 (SEQ ID NO:595) is encoded by the following transcript(s): D11853_PEA_{—}1_T19 (SEQ ID NO:68), for which the sequence(s) is/are given at the end of the application. The coding portion of transcript D11853_PEA_{—}1_T19 (SEQ ID NO:68) is shown in bold; this coding portion starts at position 108 and ends at position 767. The transcript also has the following SNPs as listed in Table 20 (given according to their position on the nucleotide sequence, with the alternative nucleic acid listed; the last column indicates whether the SNP is known or not; the presence of known SNPs in variant protein D11853_PEA_{—}1_P14 (SEQ ID NO:595) sequence provides support for the deduced sequence of this variant protein according to the present invention).

TABLE 20
Nucleic acid SNPs

| SNP position on | Alternative | Previously |
|---|---|---|
| nucleotide sequence | nucleic acid | known SNP? |
| 180 | C -> | No |
| 182 | G -> | No |
| 185 | -> C | No |
| 201 | T -> | No |
| 640 | A -> C | No |
| 640 | A -> G | No |
| 641 | G -> | No |
| 641 | G -> A | No |
| 660 | C -> G | No |
| 667 | A -> C | No |
| 867 | G -> A | No |
| 939 | G -> A | No |
| 939 | G -> C | No |
| 956 | A -> | No |
| 956 | A -> C | No |
| 967 | A -> C | No |
| 982 | C -> | No |
| 982 | C -> G | No |
| 1052 | A -> C | No |
| 1076 | A -> C | No |
| 1084 | T -> G | No |
| 1102 | G -> A | No |
| 1102 | G -> C | No |
| 1110 | -> C | No |
| 1110 | -> T | No |
| 1117 | -> G | No |
| 1146 | C -> | No |
| 1146 | C -> G | No |
| 1177 | -> G | No |
| 1227 | G -> | No |
| 1236 | G -> A | No |
| 1256 | C -> T | No |
| 1271 | G -> | No |
| 1352 | G -> A | No |
| 1355 | A -> | No |
| 1355 | A -> C | No |
| 1401 | T -> | No |
| 1404 | T -> C | No |
| 1404 | T -> G | No |
| 1441 | T -> C | Yes |

Variant protein D11853_PEA_{—}1_P16 (SEQ ID NO:596) according to the present invention has an amino acid sequence as given at the end of the application; it is encoded by transcript(s) D11853_PEA_{—}1_T24 (SEQ ID NO:71). An alignment is given to the known protein (Membrane associated protein SLP-2 (SEQ ID NO:637)) at the end of the application. One or more alignments to one or more previously published protein sequences are given at the end of the application. A brief description of the relationship of the variant protein according to the present invention to each such aligned protein is as follows:

Comparison Report Between D11853_PEA_{—}1_P16 (SEQ ID NO:596) and Q9P042 (SEQ ID NO:639):

1. An isolated chimeric polypeptide encoding for D11853_PEA_{—}1_P16 (SEQ ID NO:596), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPR (SEQ ID NO:1530) corresponding to amino acids 1-26 of D11853_PEA_{—}1_P16 (SEQ ID NO:596), a second amino acid sequence being at least 90% homologous to RASSGLPRNTVVLFVPQQEAWVVERMGRFHRILEPGLNILIPVLDRIRYVQSLKEIVINVPEQSAVTLDNVT LQIDGVLYLRIMDPYKASYGVEDPEYAVTQLAQTTMRSELGKLSLDKVFR corresponding to amino acids 13-134 of Q9P042 (SEQ ID NO:639), which also corresponds to amino acids 27-148 of D11853_PEA_{—}1_P16 (SEQ ID NO:596), a third amino acid sequence being at least 90% homologous to VEAERRKR corresponding to amino acids 180-187 of Q9P042 (SEQ ID NO:639), which also corresponds to amino acids 149-156 of D11853_PEA_{—}1_P16 (SEQ ID NO:596), a bridging amino acid A corresponding to amino acid 157 of D11853_PEA_{—}1_P16 (SEQ ID NO:596), and a fourth amino acid sequence being at least 90% homologous to TVLESEGTRESAINVAEGKKQAQILASEAEKAEQINQAAGEASAVLAKAKAKAEAIRILAAALTQHNGDA AASLTVAEQYVSAFSKLAKDSNTILLPSNPGDVTSMVAQAMGVYGALTKAPVPGTPDSLSSGSSRDVQGT DASLDEELDRVKMS corresponding to amino acids 189-342 of Q9P042 (SEQ ID NO:639), which also corresponds to amino acids 158-311 of D11853_PEA_{—}1_P16 (SEQ ID NO:596), wherein said first amino acid sequence, second amino acid sequence, third amino acid sequence, bridging amino acid and fourth amino acid sequence are contiguous and in a sequential order.

2. An isolated polypeptide encoding for a head of D11853_PEA_{—}1_P16 (SEQ ID NO:596), comprising a polypeptide being at least 70%, optionally at least about 80%, preferably at least about 85%, more preferably at least about 90% and most preferably at least about 95% homologous to the sequence MLARAARGTGALLLRGSLLASGRAPR (SEQ ID NO:1530) of D11853_PEA_{—}1_P16 (SEQ ID NO:596).

3. An isolated chimeric polypeptide encoding for an edge portion of D11853_PEA_{—}1_P16 (SEQ ID NO:596) comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RV, having a structure as follows: a sequence starting from any of amino acid numbers 148−x to 148; and ending at any of amino acid numbers 149+((n−2)−x), in which x varies from 0 to n−2.

Comparison Report Between D11853_PEA_{—}1_P16 (SEQ ID NO:596) and BAC85377 (SEQ ID NO:640):

1. An isolated chimeric polypeptide encoding for D11853_PEA_{—}1_P16 (SEQ ID NO:596), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRI corresponding to amino acids 1-109 of D11853_PEA_{—}1_P16 (SEQ ID NO:596), a second amino acid sequence being at least 90% homologous to MDPYKASYGVEDPEYAVTQLAQTTMRSELGKLSLDKVFR corresponding to amino acids 1-39 of BAC85377 (SEQ ID NO:640), which also corresponds to amino acids 110-148 of D11853_PEA_{—}1_P16 (SEQ ID NO:596), a third amino acid sequence being at least 90% homologous to VEAERRKRATVLESEGTRESAINVAEGKKQAQILASEAEKAEQINQAAGEASAVLAKAKAKAEAIRILAA ALTQH corresponding to amino acids 85-159 of BAC85377 (SEQ ID NO:640), which also corresponds to amino acids 149-223 of D11853_PEA_{—}1_P16 (SEQ ID NO:596), and a fourth amino acid sequence being at least 70%, optionally at least 80%, preferably at least 85%, more preferably at least 90% and most preferably at least 95% homologous to a polypeptide having the sequence NGDAAASLTVAEQYVSAFSKLAKDSNTILLPSNPGDVTSMVAQAMGVYGALTKAPVPGTPDSLSSGSSRD VQGTDASLDEELDRVKMS (SEQ ID NO:1531) corresponding to amino acids 224-311 of D11853_PEA_{—}1_P16 (SEQ ID NO:596), wherein said first amino acid sequence, second amino acid sequence, third amino acid sequence and fourth amino acid sequence are contiguous and in a sequential order.

2. An isolated polypeptide encoding for a head of D11853_PEA_{—}1_P16 (SEQ ID NO:596), comprising a polypeptide being at least 70%, optionally at least about 80%, preferably at least about 85%, more preferably at least about 90% and most preferably at least about 95% homologous to the sequence MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRI of D11853_PEA_{—}1_P16 (SEQ ID NO:596).

3. An isolated chimeric polypeptide encoding for an edge portion of D11853_PEA_{—}1_P16 (SEQ ID NO:596) comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RV, having a structure as follows: a sequence starting from any of amino acid numbers 148−x to 148; and ending at any of amino acid numbers 149+((n−2)−x), in which x varies from 0 to n−2.

4. An isolated polypeptide encoding for a tail of D11853_PEA_{—}1_P16 (SEQ ID NO:596), comprising a polypeptide being at least 70%, optionally at least about 80%, preferably at least about 85%, more preferably at least about 90% and most preferably at least about 95% homologous to the sequence NGDAAASLTVAEQYVSAFSKLAKDSNTILLPSNPGDVTSMVAQAMGVYGALTKAPVPGTPDSLSSGSSRD VQGTDASLDEELDRVKMS (SEQ ID NO:1531) in D11853_PEA_{—}1_P16 (SEQ ID NO:596).

Comparison Report Between D11853_PEA_{—}1_P16 (SEQ ID NO:596) and Q96FY2 (SEQ ID NO:638):

1. An isolated chimeric polypeptide encoding for D11853_PEA_{—}1_P16 (SEQ ID NO:596), comprising a first amino acid sequence being at least 90% homologous to MLARAARGTGALLLRGSLLASGRAPRRASSGLPRNTVLFVPQQEAWVVERMGRFHRILEPGLNILIPVLD RIRYVQSLKEIVINVPEQSAVTLDNVTLQIDGVLYLRIMDPYKASYGVEDPEYAVTQ corresponding to amino acids 1-128 of Q96FY2 (SEQ ID NO:638), which also corresponds to amino acids 1-128 of D11853_PEA_{—}1_P16 (SEQ ID NO:596), a bridging amino acid L corresponding to amino acid 129 of D11853_PEA_{—}1_P16 (SEQ ID NO:596), a second amino acid sequence being at least 90% homologous to AQTTMRSELGKLSLDKVFR corresponding to amino acids 130-148 of Q96FY2 (SEQ ID NO:638), which also corresponds to amino acids 130-148 of D11853_PEA_{—}1_P16 (SEQ ID NO:596), and a third amino acid sequence being at least 90% homologous to VEAERRKRATVLESEGTRESAINVAEGKKQAQILASEAEKAEQINQAAGEASAVLAKAKAKAEAIRILAA ALTQHNGDAAASLTVAEQYVSAFSKLAKDSNTILLPSNPGDVTSMVAQAMGVYGALTKAPVPGTPDSLSS GSSRDVQGTDASLDEELDRVKMS corresponding to amino acids 194-356 of Q96FY2 (SEQ ID NO:638), which also corresponds to amino acids 149-311 of D11853_PEA_{—}1_P16 (SEQ ID NO:596), wherein said first amino acid sequence, bridging amino acid, second amino acid sequence and third amino acid sequence are contiguous and in a sequential order.

2. An isolated chimeric polypeptide encoding for an edge portion of D11853_PEA_{—}1_P16 (SEQ ID NO:596) comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RV, having a structure as follows: a sequence starting from any of amino acid numbers 148−x to 148; and ending at any of amino acid numbers 149+((n−2)−x), in which x varies from 0 to n−2.

Comparison Report Between D11853_PEA_{—}1_P16 (SEQ ID NO:596) and Q9UJZ1 (SEQ ID NO:637):

1. An isolated chimeric polypeptide encoding for D11853_PEA_{—}1_P16 (SEQ ID NO:596), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQLAQTTMRSELGKLS LDKVFR corresponding to amino acids 1-148 of Q9UJZ1 (SEQ ID NO:637), which also corresponds to amino acids 1-148 of D11853_PEA_{—}1_P16 (SEQ ID NO:596), and a second amino acid sequence being at least 90% homologous to VEAERRKRATVLESEGTRESAINVAEGKKQAQILASEAEKAEQINQAAGEASAVLAKAKAKAEAIRILAA ALTQHNGDAAASLTVAEQYVSAFSKLAKDSNTILLPSNPGDVTSMVAQAMGVYGALTKAPVPGTPDSLSS GSSRDVQGTDASLDEELDRVKMS corresponding to amino acids 194-356 of Q9UJZ1 (SEQ ID NO:637), which also corresponds to amino acids 149-311 of D11853_PEA_{—}1_P16 (SEQ ID NO:596), wherein said first amino acid sequence and second amino acid sequence are contiguous and in a sequential order.

2. An isolated chimeric polypeptide encoding for an edge portion of D11853_PEA_{—}1_P16 (SEQ ID NO:596), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RV, having a structure as follows: a sequence starting from any of amino acid numbers 148−x to 148; and ending at any of amino acid numbers 149+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P16 (SEQ ID NO:596) also has the following non-silent SNPs (Single Nucleotide Polymorphisms) as listed in Table 21, (given according to their position(s) on the amino acid sequence, with the alternative amino acid(s) listed; the last column indicates whether the SNP is known or not; the presence of known SNPs in variant protein D11853_PEA_{—}1_P16 (SEQ ID NO:596) sequence provides support for the deduced sequence of this variant protein according to the present invention).

TABLE 21
Amino acid mutations

| SNP position(s) on | Alternative | Previously |
|---|---|---|
| amino acid sequence | amino acid(s) | known SNP? |
| 25 | P -> | No |
| 32 | L -> | No |
| 161 | E -> K | No |
| 185 | E -> K | No |
| 185 | E -> Q | No |
| 190 | E -> | No |
| 190 | E -> D | No |
| 194 | Q -> P | No |
| 199 | A -> | No |
| 199 | A -> G | No |
| 222 | Q -> H | No |
| 233 | V -> G | No |
| 239 | S -> N | No |
| 239 | S -> T | No |
| 254 | P -> | No |
| 254 | P -> A | No |
| 281 | G -> | No |
| 284 | D -> N | No |
| 295 | Q -> | No |

Variant protein D11853_PEA_{—}1_P16 (SEQ ID NO:596) is encoded by the following transcript(s): D11853_PEA_{—}1_T24 (SEQ ID NO:71), for which the sequence(s) is/are given at the end of the application. The coding portion of transcript D11853_PEA_{—}1_T24 (SEQ ID NO:71) is shown in bold; this coding portion starts at position 108 and ends at position 1040. The transcript also has the following SNPs as listed in Table 22 (given according to their position on the nucleotide sequence, with the alternative nucleic acid listed; the last column indicates whether the SNP is known or not; the presence of known SNPs in variant protein D11853_PEA_{—}1_P16 (SEQ ID NO:596) sequence provides support for the deduced sequence of this variant protein according to the present invention).

TABLE 22
Nucleic acid SNPs

| SNP position on | Alternative | Previously |
|---|---|---|
| nucleotide sequence | nucleic acid | known SNP? |
| 180 | C -> | No |
| 182 | G -> | No |
| 185 | -> C | No |
| 201 | T -> | No |
| 588 | G -> A | No |
| 660 | G -> A | No |
| 660 | G -> C | No |
| 677 | A -> | No |
| 677 | A -> C | No |
| 688 | A -> C | No |
| 703 | C -> | No |
| 703 | C -> G | No |
| 773 | A -> C | No |
| 797 | A -> C | No |
| 805 | T -> G | No |
| 823 | G -> A | No |
| 823 | G -> C | No |
| 831 | -> C | No |
| 831 | -> T | No |
| 838 | -> G | No |
| 867 | C -> | No |
| 867 | C -> G | No |
| 898 | -> G | No |
| 948 | G -> | No |
| 957 | G -> A | No |
| 977 | C -> T | No |
| 992 | G -> | No |
| 1073 | G -> A | No |
| 1076 | A -> | No |
| 1076 | A -> C | No |
| 1122 | T -> | No |
| 1125 | T -> C | No |
| 1125 | T -> G | No |
| 1162 | T -> C | Yes |

Variant protein D11853_PEA_{—}1_P18 (SEQ ID NO:597) according to the present invention has an amino acid sequence as given at the end of the application; it is encoded by transcript(s) D11853_PEA_{—}1_T26 (SEQ ID NO:73). An alignment is given to the known protein (Membrane associated protein SLP-2 (SEQ ID NO:637)) at the end of the application. One or more alignments to one or more previously published protein sequences are given at the end of the application. A brief description of the relationship of the variant protein according to the present invention to each such aligned protein is as follows:

Comparison Report Between D11853_PEA_{—}1_P18 (SEQ ID NO:597) and Q9P042 (SEQ ID NO:639):

1. An isolated chimeric polypeptide encoding for D11853_PEA_{—}1_P18 (SEQ ID NO:597), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPR (SEQ ID NO:1530) corresponding to amino acids 1-26 of D11853_PEA_{—}1_P18 (SEQ ID NO:597), a second amino acid sequence being at least 90% homologous to RASSGLPRNTVVLFVPQQEAWVVERMGRFHRILEPGLNILIPVLDRIRYVQSLKEIVINVPEQSAVTLDNVT LQIDGVLYLRIMDPYKASYGVEDPEYAVTQLAQTTMRSELGKLSLDKVFRERESLNASI corresponding to amino acids 13-143 of Q9P042 (SEQ ID NO:639), which also corresponds to amino acids 27-157 of D11853_PEA_{—}1_P18 (SEQ ID NO:597), and a third amino acid sequence being at least 90% homologous to VAQAMGVYGALTKAPVPGTPDSLSSGSSRDVQGTDASLDEELDRVKMS corresponding to amino acids 295-342 of Q9P042 (SEQ ID NO:639), which also corresponds to amino acids 158-205 of D11853_PEA_{—}1_P18 (SEQ ID NO:597), wherein said first amino acid sequence, second amino acid sequence and third amino acid sequence are contiguous and in a sequential order.

2. An isolated polypeptide encoding for a head of D11853_PEA_{—}1_P18 (SEQ ID NO:597), comprising a polypeptide being at least 70%, optionally at least about 80%, preferably at least about 85%, more preferably at least about 90% and most preferably at least about 95% homologous to the sequence MLARAARGTGALLLRGSLLASGRAPR (SEQ ID NO:1530) of D11853_PEA_{—}1_P18 (SEQ ID NO:597).

3. An isolated chimeric polypeptide encoding for an edge portion of D11853_PEA_{—}1_P18 (SEQ ID NO:597) comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IV, having a structure as follows: a sequence starting from any of amino acid numbers 157−x to 157; and ending at any of amino acid numbers 158+((n−2)−x), in which x varies from 0 to n−2.

Comparison Report Between D11853_PEA_{—}1_P18 (SEQ ID NO:597) and Q96FY2 (SEQ ID NO:638):

1. An isolated chimeric polypeptide encoding for D11853_PEA_{—}1_P18 (SEQ ID NO:597), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQ corresponding to amino acids 1-128 of Q96FY2 (SEQ ID NO:638), which also corresponds to amino acids 1-128 of D11853_PEA_{—}1_P18 (SEQ ID NO:597), a bridging amino acid L corresponding to amino acid 129 of D11853_PEA_{—}1_P18 (SEQ ID NO:597), a second amino acid sequence being at least 90% homologous to AQTTMRSELGKLSLDKVFRERESLNASI corresponding to amino acids 130-157 of Q96FY2 (SEQ ID NO:638), which also corresponds to amino acids 130-157 of D11853_PEA_{—}1_P18 (SEQ ID NO:597), and a third amino acid sequence being at least 90% homologous to VAQAMGVYGALTKAPVPGTPDSLSSGSSRDVQGTDASLDEELDRVKMS corresponding to amino acids 309-356 of Q96FY2 (SEQ ID NO:638), which also corresponds to amino acids 158-205 of D11853_PEA_{—}1_P18 (SEQ ID NO:597), wherein said first amino acid sequence, bridging amino acid, second amino acid sequence and third amino acid sequence are contiguous and in a sequential order.

2. An isolated chimeric polypeptide encoding for an edge portion of D11853_PEA_{—}1_P18 (SEQ ID NO:597) comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IV, having a structure as follows: a sequence starting from any of amino acid numbers 157−x to 157; and ending at any of amino acid numbers 158+((n−2)−x), in which x varies from 0 to n−2.

Comparison Report Between D11853_PEA_{—}1_P18 (SEQ ID NO:597) and Q9UJZ1 (SEQ ID NO:637):

1. An isolated chimeric polypeptide encoding for D11853_PEA_{—}1_P18 (SEQ ID NO:597), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQLAQTTMRSELGKLS LDKVFRERESLNASI corresponding to amino acids 1-157 of Q9UJZ1 (SEQ ID NO:637), which also corresponds to amino acids 1-157 of D11853_PEA_{—}1_P18 (SEQ ID NO:597), and a second amino acid sequence being at least 90% homologous to VAQAMGVYGALTKAPVPGTPDSLSSGSSRDVQGTDASLDEELDRVKMS corresponding to amino acids 309-356 of Q9UJZ1 (SEQ ID NO:637), which also corresponds to amino acids 158-205 of D11853_PEA_{—}1_P18 (SEQ ID NO:597), wherein said first amino acid sequence and second amino acid sequence are contiguous and in a sequential order.

2. An isolated chimeric polypeptide encoding for an edge portion of D11853_PEA_{—}1_P18 (SEQ ID NO:597) comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise IV, having a structure as follows: a sequence starting from any of amino acid numbers 157−x to 157; and ending at any of amino acid numbers 158+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P18 (SEQ ID NO:597) also has the following non-silent SNPs (Single Nucleotide Polymorphisms) as listed in Table 23, (given according to their position(s) on the amino acid sequence, with the alternative amino acid(s) listed; the last column indicates whether the SNP is known or not; the presence of known SNPs in variant protein D11853_PEA_{—}1_P18 (SEQ ID NO:597) sequence provides support for the deduced sequence of this variant protein according to the present invention).

TABLE 23
Amino acid mutations

| SNP position(s) on | Alternative | Previously |
|---|---|---|
| amino acid sequence | amino acid(s) | known SNP? |
| 25 | P -> | No |
| 32 | L -> | No |
| 175 | G -> | No |
| 178 | D -> N | No |
| 189 | Q -> | No |

Variant protein D11853_PEA_{—}1_P18 (SEQ ID NO:597) is encoded by the following transcript(s): D11853_PEA_{—}1_T26 (SEQ ID NO:73), for which the sequence(s) is/are given at the end of the application. The coding portion of transcript D11853_PEA_{—}1_T26 (SEQ ID NO:73) is shown in bold; this coding portion starts at position 108 and ends at position 722. The transcript also has the following SNPs as listed in Table 24 (given according to their position on the nucleotide sequence, with the alternative nucleic acid listed; the last column indicates whether the SNP is known or not; the presence of known SNPs in variant protein D11853_PEA_{—}1_P18 (SEQ ID NO:597) sequence provides support for the deduced sequence of this variant protein according to the present invention).

TABLE 24
Nucleic acid SNPs

| SNP position on | Alternative | Previously |
|---|---|---|
| nucleotide sequence | nucleic acid | known SNP? |
| 180 | C -> | No |
| 182 | G -> | No |
| 185 | -> C | No |
| 201 | T -> | No |
| 630 | G -> | No |
| 639 | G -> A | No |
| 659 | C -> T | No |
| 674 | G -> | No |
| 755 | G -> A | No |
| 758 | A -> | No |
| 758 | A -> C | No |
| 804 | T -> | No |
| 807 | T -> C | No |
| 807 | T -> G | No |
| 844 | T -> C | Yes |

Variant protein D11853_PEA_{—}1_P19 (SEQ ID NO:598) according to the present invention has an amino acid sequence as given at the end of the application; it is encoded by transcript(s) D11853_PEA_{—}1_T27 (SEQ ID NO:74). An alignment is given to the known protein (Membrane associated protein SLP-2 (SEQ ID NO:637)) at the end of the application. One or more, alignments to one or more previously published protein sequences are given at the end of the application. A brief description of the relationship of the variant protein according to the present invention to each such aligned protein is as follows:

Comparison Report Between D11853_PEA_{—}1_P19 (SEQ ID NO:598) and Q9P042 (SEQ ID NO:639):

1. An isolated chimeric polypeptide encoding for D11853_PEA_{—}1_P19 (SEQ ID NO:598), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPR (SEQ ID NO:1530) corresponding to amino acids 1-26 of D11853_PEA_{—}1_P19 (SEQ ID NO:598), a second amino acid sequence being at least 90% homologous to RASSGLPRNTVVLFVPQQEAWVVERMGRFHRILEPGLNILIPVLDRIRYVQSLKEIVINVPEQSAVTLDNVT LQIDGVLYLRIMDPYKASYGVEDPEYAVTQLAQTTMRSELGKLS corresponding to amino acids 13-128 of Q9P042 (SEQ ID NO:639), which also corresponds to amino acids 27-142 of D11853_PEA_{—}1_P19 (SEQ ID NO:598), and a third amino acid sequence being at least 70%, optionally at least 80%, preferably at least 85%, more preferably at least 90% and most preferably at least 95% homologous to a polypeptide having the sequence SRLTLQWEQQRCPGYRCKS (SEQ ID NO:1552) corresponding to amino acids 143-161 of D11853_PEA_{—}1_P19 (SEQ ID NO:598), wherein said first amino acid sequence, second amino acid sequence and third amino acid sequence are contiguous and in a sequential order.

2. An isolated polypeptide encoding for a head of D11853_PEA_{—}1_P19 (SEQ ID NO:598), comprising a polypeptide being at least 70%, optionally at least about 80%, preferably at least about 85%, more preferably at least about 90% and most preferably at least about 95% homologous to the sequence MLARAARGTGALLLRGSLLASGRAPR (SEQ ID NO:1530) of D11853_PEA_{—}1_P19 (SEQ ID NO:598).

3. An isolated polypeptide encoding for a tail of D11853_PEA_{—}1_P19 (SEQ ID NO:598), comprising a polypeptide being at least 70%, optionally at least about 80%, preferably at least about 85%, more preferably at least about 90% and most preferably at least about 95% homologous to the sequence SRLTLQWEQQRCPGYRCKS (SEQ ID NO:1552) in D11853_PEA_{—}1_P19 (SEQ ID NO:598).

Comparison Report Between D11853_PEA_{—}1_P19 (SEQ ID NO:598) and Q96FY2 (SEQ ID NO:638):

1. An isolated chimeric polypeptide encoding for D11853_PEA_{—}1_P19 (SEQ ID NO:598), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTLDNVTLQIDGVLYLRIMDPYKASYGVEDPEYAVTQ corresponding to amino acids 1-128 of Q96FY2 (SEQ ID NO:638), which also corresponds to amino acids 1-128 of D11853_PEA_{—}1_P19 (SEQ ID NO:598), a bridging amino acid L corresponding to amino acid 129 of D11853_PEA_{—}1_P19 (SEQ ID NO:598), a second amino acid sequence being at least 90% homologous to AQTTMRSELGKLS corresponding to amino acids 130-142 of Q96FY2 (SEQ ID NO:638), which also corresponds to amino acids 130-142 of D11853_PEA_{—}1_P19 (SEQ ID NO:598), and a third amino acid sequence being at least 70%, optionally at least 80%, preferably at least 85%, more preferably at least 90% and most preferably at least 95% homologous to a polypeptide having the sequence SRLTLQWEQQRCPGYRCKS (SEQ ID NO:1552) corresponding to amino acids 143-161 of D11853_PEA_{—}1_P19 (SEQ ID NO:598), wherein said first amino acid sequence, bridging amino acid, second amino acid sequence and third amino acid sequence are contiguous and in a sequential order.

2. An isolated polypeptide encoding for a tail of D11853_PEA_{—}1_P19 (SEQ ID NO:598), comprising a polypeptide being at least 70%, optionally at least about 80%, preferably at least about 85%, more preferably at least about 90% and most preferably at least about 95% homologous to the sequence SRLTLQWEQQRCPGYRCKS (SEQ ID NO:1552) in D11853_PEA_{—}1_P19 (SEQ ID NO:598).

Comparison Report Between D11853_PEA_{—}1_P19 (SEQ ID NO:598) and Q9UJZ1 (SEQ ID NO:637):

1. An isolated chimeric polypeptide encoding for D11853_PEA_{—}1_P19 (SEQ ID NO:598), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEWINVPEQSAVTLDNVTLQIDGVLYLRIMDPYKASYGVEDPEYAVTQLAQTTMRSELGKLS corresponding to amino acids 1-142 of Q9UJZ1 (SEQ ID NO:637), which also corresponds to amino acids 1-142 of D11853_PEA_{—}1_P19 (SEQ ID NO:598), and a second amino acid sequence being at least 70%, optionally at least 80%, preferably at least 85%, more preferably at least 90% and most preferably at least 95% homologous to a polypeptide having the sequence SRLTLQWEQQRCPGYRCKS (SEQ ID NO:1552) corresponding to amino acids 143-161 of D11853_PEA_{—}1_P19 (SEQ ID NO:598), wherein said first amino acid sequence and second amino acid sequence are contiguous and in a sequential order.

2. An isolated polypeptide encoding for a tail of D11853_PEA_{—}1_P19 (SEQ ID NO:598), comprising a polypeptide being at least 70%, optionally at least about 80%, preferably at least about 85%, more preferably at least about 90% and most preferably at least about 95% homologous to the sequence SRLTLQWEQQRCPGYRCKS (SEQ ID NO:1552) in D11853_PEA_{—}1_P19 (SEQ ID NO:598).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P19 (SEQ ID NO:598) also has the following non-silent SNPs (Single Nucleotide Polymorphisms) as listed in Table 25, (given according to their position(s) on the amino acid sequence, with the alternative amino acid(s) listed; the last column indicates whether the SNP is known or not; the presence of known SNPs in variant protein D11853_PEA_{—}1_P19 (SEQ ID NO:598) sequence provides support for the deduced sequence of this variant protein according to the present invention).

TABLE 25
Amino acid mutations

| SNP Position(s) | | |
|---|---|---|
| on amino acid | Alternative | Previously |
| sequence | amine acid(s) | known SNP? |
| 25 | P -> | No |
| 32 | L -> | No |
| 144 | R -> K | No |
| 151 | Q -> * | No |
| 156 | G -> | No |

Variant protein D11853_PEA_{—}1_P19 (SEQ ID NO:598) is encoded by the following transcript(s): D11853_PEA_{—}1_T27 (SEQ ID NO:74), for which the sequence(s) is/are given at the end of the application. The coding portion of transcript D11853_PEA_{—}1_T27 (SEQ ID NO:74) is shown in bold; this coding portion starts at position 108 and ends at position 590. The transcript also has the following SNPs as listed in Table 26 (given according to their position on the nucleotide sequence, with the alternative nucleic acid listed; the last column indicates whether the SNP is known or not; the presence of known SNPs in variant protein D11853_PEA_{—}1_P19 (SEQ ID NO:598) sequence provides support for the deduced sequence of this variant protein according to the present invention).

TABLE 26
Nucleic acid SNPs

| SNP position | | |
|---|---|---|
| on nucleotide | Alternative | Previously |
| sequence | nucleic acid | known SNP? |
| 180 | C -> | No |
| 182 | G -> | No |
| 185 | -> C | No |
| 201 | T -> | No |
| 538 | G -> A | No |
| 558 | C -> T | No |
| 573 | G -> | No |
| 654 | G -> A | No |
| 657 | A -> | No |
| 657 | A -> C | No |
| 703 | T -> | No |
| 706 | T -> C | No |
| 706 | T -> G | No |
| 743 | T -> C | Yes |

Variant protein D11853_PEA_{—}1_P20 (SEQ ID NO:599) according to the present invention has an amino acid sequence as given at the end of the application; it is encoded by transcript(s) D11853_PEA_{—}1_T7 (SEQ ID NO:59), D11853_PEA_{—}1_T17 (SEQ ID NO:67) and D11853_PEA_{—}1_T25 (SEQ ID NO:72). The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P20 (SEQ ID NO:599) is encoded by the following transcript(s): D11853_PEA_{—}1_T7 (SEQ ID NO:59), D11853_PEA_{—}1_T17 (SEQ ID NO:67) and D11853_PEA_{—}1_T25 (SEQ ID NO:72), for which the sequence(s) is/are given at the end of the application.

The coding portion of transcript D11853_PEA_{—}1_T7 (SEQ ID NO:59) is shown in bold; this coding portion starts at position 108 and ends at position 287. The transcript also has the following SNPs as listed in Table 27 (given according to their position on the nucleotide sequence, with the alternative nucleic acid listed; the last column indicates whether the SNP is known or not; the presence of known SNPs in variant protein D11853_PEA_{—}1_P20 (SEQ ID NO:599) sequence provides support for the deduced sequence of this variant protein according to the present invention).

TABLE 27
Nucleic acid SNPs

| SNP position | | |
|---|---|---|
| on nucleotide | Alternative | Previously |
| sequence | nucleic acid | known SNP? |
| 406 | C -> | No |
| 408 | G -> | No |
| 411 | -> C | No |
| 427 | T -> | No |
| 1357 | A -> C | No |
| 1357 | A -> G | No |
| 1358 | G -> | No |
| 1358 | G -> A | No |
| 1377 | C -> G | No |
| 1384 | A -> C | No |
| 1440 | G -> A | No |
| 1512 | G -> A | No |
| 1512 | G -> C | No |
| 1529 | A -> | No |
| 1529 | A -> C | No |
| 1540 | A -> C | No |
| 1555 | C -> | No |
| 1555 | C -> G | No |
| 1625 | A -> C | No |
| 1649 | A -> C | No |
| 1657 | T -> G | No |
| 1675 | G -> A | No |
| 1675 | G -> C | No |
| 1683 | -> C | No |
| 1683 | -> T | No |
| 1690 | -> G | No |
| 1719 | C -> | No |
| 1719 | C -> G | No |
| 1750 | -> G | No |
| 1800 | G -> | No |
| 1809 | G -> A | No |
| 1829 | C -> T | No |
| 1844 | G -> | No |
| 1925 | G -> A | No |
| 1928 | A -> | No |
| 1928 | A -> C | No |
| 1974 | T -> | No |
| 1977 | T -> C | No |
| 1977 | T -> G | No |
| 2014 | T -> C | Yes |

The coding portion of transcript D11853_PEA_{—}1_T17 (SEQ ID NO:67) is shown in bold; this coding portion starts at position 108 and ends at position 287. The transcript also has the following SNPs as listed in Table 28 (given according to their position on the nucleotide sequence, with the alternative nucleic acid listed; the last column indicates whether the SNP is known or not; the presence of known SNPs in variant protein D11853_PEA_{—}1_P20 (SEQ ID NO:599) sequence provides support for the deduced sequence of this variant protein according to the present invention).

TABLE 28
Nucleic acid SNPs

| SNP position | | |
|---|---|---|
| on nucleotide | Alternative | Previously |
| sequence | nucleic acid | known SNP? |
| 406 | C -> | No |
| 408 | G -> | No |
| 411 | -> C | No |
| 427 | T -> | No |
| 1357 | A -> C | No |
| 1357 | A -> G | No |
| 1358 | G -> | No |
| 1358 | G -> A | No |
| 1377 | C -> G | No |
| 1384 | A -> C | No |
| 1440 | G -> A | No |
| 1512 | G -> A | No |
| 1512 | G -> C | No |
| 1529 | A -> | No |
| 1529 | A -> C | No |
| 1540 | A -> C | No |
| 1555 | C -> | No |
| 1555 | C -> G | No |
| 1625 | A -> C | No |
| 1649 | A -> C | No |
| 1657 | T -> G | No |
| 1675 | G -> A | No |
| 1675 | G -> C | No |
| 1683 | -> C | No |
| 1683 | -> T | No |
| 1690 | -> G | No |
| 1719 | C -> | No |
| 1719 | C -> G | No |
| 1750 | -> G | No |
| 1800 | G -> | No |
| 1809 | G -> A | No |
| 1829 | C -> T | No |
| 1844 | G -> | No |
| 1925 | G -> A | No |
| 1928 | A -> | No |
| 1928 | A -> C | No |
| 1974 | T -> | No |
| 1977 | T -> C | No |
| 1977 | T -> G | No |
| 2014 | T -> C | Yes |

The coding portion of transcript D11853_PEA_{—}1_T25 (SEQ ID NO:72) is shown in bold; this coding portion starts at position 108 and ends at position 287. The transcript also has the following SNPs as listed in Table 29 (given according to their position on the nucleotide sequence, with the alternative nucleic acid listed; the last column indicates whether the SNP is known or not; the presence of known SNPs in variant protein D11853_PEA_{—}1_P20 (SEQ ID NO:599) sequence provides support for the deduced sequence of this variant protein according to the present invention).

TABLE 29
Nucleic acid SNPs

| SNP position | | |
|---|---|---|
| on nucleotide | Alternative | Previously |
| sequence | nucleic acid | known SNP? |
| 406 | C -> | No |
| 408 | G -> | No |
| 411 | -> C | No |
| 427 | T -> | No |
| 1489 | A -> C | No |
| 1489 | A -> G | No |
| 1490 | G -> | No |
| 1490 | G -> A | No |
| 1509 | C -> G | No |
| 1516 | A -> C | No |
| 1572 | G -> A | No |
| 1644 | G -> A | No |
| 1644 | G -> C | No |
| 1661 | A -> | No |
| 1661 | A -> C | No |
| 1672 | A -> C | No |
| 1687 | C -> | No |
| 1687 | C -> G | No |
| 1757 | A -> C | No |
| 1986 | A -> C | No |
| 1994 | T -> G | No |
| 2012 | G -> A | No |
| 2012 | G -> C | No |
| 2020 | -> C | No |
| 2020 | -> T | No |
| 2027 | -> G | No |
| 2056 | C -> | No |
| 2056 | C -> G | No |
| 2087 | -> G | No |
| 2120 | C -> T | No |
| 2562 | G -> | No |
| 2571 | G -> A | No |
| 2591 | C -> T | No |
| 2606 | G -> | No |
| 2687 | G -> A | No |
| 2690 | A -> | No |
| 2690 | A -> C | No |
| 2736 | T -> | No |
| 2739 | T -> C | No |
| 2739 | T -> G | No |
| 2776 | T -> C | Yes |

Variant protein D11853_PEA_{—}1_P21 (SEQ ID NO:600) according to the present invention has an amino acid sequence as given at the end of the application; it is encoded by transcript(s) D11853_PEA_{—}1_T8 (SEQ ID NO:60). An alignment is given to the known protein (Membrane associated protein SLP-2 (SEQ ID NO:637)) at the end of the application. One or more alignments to one or more previously published protein sequences are given at the end of the application. A brief description of the relationship of the variant protein according to the present invention to each such aligned protein is as follows:

Comparison Report Between D11853_PEA_{—}1_P21 (SEQ ID NO:600) and Q96FY2 (SEQ ID NO:638):

1. An isolated chimeric polypeptide encoding for D11853_PEA_{—}1_P21 (SEQ ID NO:600), comprising a first amino acid sequence being at least 90% homologous to MLARAARGTGALLLRGSLLASGRAPRRASSGLPRNTVVLFVPQQEAWVVERMGRFHRILEP corresponding to amino acids 1-61 of Q96FY2 (SEQ ID NO:638), which also corresponds to amino acids 1-61 of D11853_PEA_{—}1_P21 (SEQ ID NO:600), and a second amino acid sequence being at least 70%, optionally at least 80%, preferably at least 85%, more preferably at least 90% and most preferably at least 95% homologous to a polypeptide having the sequence VRNLFCPPWASQMTNPSRHAMSGGLPLGLPALLAPDSVGQT (SEQ ID NO:1553) corresponding to amino acids 62-102 of D11853_PEA_{—}1_P21 (SEQ ID NO:600), wherein said first amino acid sequence and second amino acid sequence are contiguous and in a sequential order.

2. An isolated polypeptide encoding for a tail of D11853_PEA_{—}1_P21 (SEQ ID NO:600), comprising a polypeptide being at least 70%, optionally at least about 80%, preferably at least about 85%, more preferably at least about 90% and most preferably at least about 95% homologous to the sequence VRNLFCPPWASQMTNPSRHAMSGGLPLGLPALLAPDSVGQT (SEQ ID NO:1553) in D11853_PEA_{—}1_P21 (SEQ ID NO:600).

Comparison Report Between D11853_PEA_{—}1_P21 (SEQ ID NO:600) and Q9UJZ1 (SEQ ID NO:637):

1. An isolated chimeric polypeptide encoding for D11853_PEA_{—}1_P21 (SEQ ID NO:600), comprising a first amino acid sequence being at least 90% homologous to MLARAARGTGALLLRGSLLASGRAPRRASSGLPRNTVVLFVPQQEAWVVERMGRFHRILEP corresponding to amino acids 1-61 of Q9UJZ1 (SEQ ID NO:637), which also corresponds to amino acids 1-61 of D11853_PEA_{—}1_P21 (SEQ ID NO:600), and a second amino acid sequence being at least 70%, optionally at least 80%, preferably at least 85%, more preferably at least 90% and most preferably at least 95% homologous to a polypeptide having the sequence VRNLFCPPWASQMTNPSRHAMSGGLPLGLPALLAPDSVGQT (SEQ ID NO:1553) corresponding to amino acids 62-102 of D11853_PEA_{—}1_P21 (SEQ ID NO:600), wherein said first amino acid sequence and second amino acid sequence are contiguous and in a sequential order.

2. An isolated polypeptide encoding for a tail of D11853_PEA_{—}1_P21 (SEQ ID NO:600), comprising a polypeptide being at least 70%, optionally at least about 80%, preferably at least about 85%, more preferably at least about 90% and most preferably at least about 95% homologous to the sequence VRNLFCPPWASQMTNPSRHAMSGGLPLGLPALLAPDSVGQT (SEQ ID NO:1553) in D11853_PEA_{—}1_P21 (SEQ ID NO:600).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P21 (SEQ ID NO:600) also has the following non-silent SNPs (Single Nucleotide Polymorphisms) as listed in Table 30, (given according to their position(s) on the amino acid sequence, with the alternative amino acid(s) listed; the last column indicates whether the SNP is known or not; the presence of known SNPs in variant protein D11853_PEA_{—}1_P21 (SEQ ID NO:600) sequence provides support for the deduced sequence of this variant protein according to the present invention).

TABLE 30
Amino acid mutations

| SNP position(s) | | |
|---|---|---|
| on amino acid | Alternative | Previously |
| sequence | amino acid(s) | known SNP? |
| 25 | P -> | No |
| 32 | L -> | No |

Variant protein D11853_PEA_{—}1_P21 (SEQ ID NO:600) is encoded by the following transcript(s): D11853_PEA_{—}1_T8 (SEQ ID NO:60), for which the sequence(s) is/are given at the end of the application. The coding portion of transcript D11853_PEA_{—}1_T8 (SEQ ID NO:60) is shown in bold; this coding portion starts at position 108 and ends at position 413. The transcript also has the following SNPs as listed in Table 31 (given according to their position on the nucleotide sequence, with the alternative nucleic acid listed; the last column indicates whether the SNP is known or not; the presence of known SNPs in variant protein D11853_PEA_{—}1_P21 (SEQ ID NO:600) sequence provides support for the deduced sequence of this variant protein according to the present invention).

TABLE 31
Nucleic acid SNPs

| SNP position | | |
|---|---|---|
| on nucleotide | Alternative | Previously |
| sequence | nucleic acid | known SNP? |
| 180 | C -> | No |
| 182 | G -> | No |
| 185 | -> C | No |
| 201 | T -> | No |
| 1131 | A -> C | No |
| 1131 | A -> G | No |
| 1132 | G -> | No |
| 1132 | G -> A | No |
| 1151 | C -> G | No |
| 1158 | A -> C | No |
| 1214 | G -> A | No |
| 1286 | G -> A | No |
| 1286 | G -> C | No |
| 1303 | A -> | No |
| 1303 | A -> C | No |
| 1314 | A -> C | No |
| 1329 | C -> | No |
| 1329 | C -> G | No |
| 1399 | A -> C | No |
| 1423 | A -> C | No |
| 1431 | T -> G | No |
| 1449 | G -> A | No |
| 1449 | G -> C | No |
| 1457 | -> C | No |
| 1457 | -> T | No |
| 1464 | -> G | No |
| 1493 | C -> | No |
| 1493 | C -> G | No |
| 1524 | -> G | No |
| 1574 | G -> | No |
| 1583 | G -> A | No |
| 1603 | C -> T | No |
| 1618 | G -> | No |
| 1699 | G -> A | No |
| 1702 | A -> | No |
| 1702 | A -> C | No |
| 1748 | T -> | No |
| 1751 | T -> C | No |
| 1751 | T -> G | No |
| 1788 | T -> C | Yes |

Variant protein D11853_PEA_{—}1_P22 (SEQ ID NO:601) according to the present invention has an amino acid sequence as given at the end of the application; it is encoded by transcript(s) D11853_PEA_{—}1_T9 (SEQ ID NO:61). An alignment is given to the known protein (Membrane associated protein SLP-2 (SEQ ID NO:637)) at the end of the application. One or more alignments to one or more previously published protein sequences are given at the end of the application. A brief description of the relationship of the variant protein according to the present invention to each such aligned protein is as follows:

Comparison Report Between D11853_PEA_{—}1_P22 (SEQ ID NO:601) and Q9P042 (SEQ ID NO:639):

1. An isolated chimeric polypeptide encoding for D11853_PEA_{—}1_P22 (SEQ ID NO:601), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPR (SEQ ID NO:1530) corresponding to amino acids 1-26 of D11853_PEA_{—}1_P22 (SEQ ID NO:601), a second amino acid sequence being at least 90% homologous to RASSGLPRNTVVLFVPQQEAWVVERMGRFHRILEP corresponding to amino acids 13-47 of Q9P042 (SEQ ID NO:639), which also corresponds to amino acids 27-61 of D11853_PEA_{—}1_P22 (SEQ ID NO:601), and a third amino acid sequence being at least 70%, optionally at least 80%, preferably at least 85%, more preferably at least 90% and most preferably at least 95% homologous to a polypeptide having the sequence ELLLFWACSMC (SEQ ID NO:1555) corresponding to amino acids 62-72 of D11853_PEA_{—}1_P22 (SEQ ID NO:601), wherein said first amino acid sequence, second amino acid sequence and third amino acid sequence are contiguous and in a sequential order.

2. An isolated polypeptide encoding for a head of D11853_PEA_{—}1_P22 (SEQ ID NO:601), comprising a polypeptide being at least 70%, optionally at least about 80%, preferably at least about 85%, more preferably at least about 90% and most preferably at least about 95% homologous to the sequence MLARAARGTGALLLRGSLLASGRAPR (SEQ ID NO:1530) of D11853_PEA_{—}1_P22 (SEQ ID NO:601).

3. An isolated polypeptide encoding for a tail of D11853_PEA_{—}1_P22 (SEQ ID NO:601), comprising a polypeptide being at least 70%, optionally at least about 80%, preferably at least about 85%, more preferably at least about 90% and most preferably at least about 95% homologous to the sequence ELLLFWACSMC (SEQ ID NO:1555) in D11853_PEA_{—}1_P22 (SEQ ID NO:601).

Comparison Report Between D11853_PEA_{—}1_P22 (SEQ ID NO:601) and Q96FY2 (SEQ ID NO:638):

1. An isolated chimeric polypeptide encoding for D11853_PEA_{—}1_P22 (SEQ ID NO:601), comprising a first amino acid sequence being at least 90% homologous to MLARAARGTGALLLRGSLLASGRAPRRASSGLPRNTVVLFVPQQEAWVVERMGRFHRILEP corresponding to amino acids 1-61 of Q96FY2 (SEQ ID NO:638), which also corresponds to amino acids 1-61 of D11853_PEA_{—}1_P22 (SEQ ID NO:601), and a second amino acid sequence being at least 70%, optionally at least 80%, preferably at least 85%, more preferably at least 90% and most preferably at least 95% homologous to a polypeptide having the sequence ELLLFWACSMC (SEQ ID NO:1555) corresponding to amino acids 62-72 of D11853_PEA_{—}1_P22 (SEQ ID NO:601), wherein said first amino acid sequence and second amino acid sequence are contiguous and in a sequential order.

2. An isolated polypeptide encoding for a tail of D11853_PEA_{—}1_P22 (SEQ ID NO:601), comprising a polypeptide being at least 70%, optionally at least about 80%, preferably at least about 85%, more preferably at least about 90% and most preferably at least about 95% homologous to the sequence ELLLFWACSMC (SEQ ID NO:1555) in D11853_PEA_{—}1_P22 (SEQ ID NO:601).

Comparison report between D11853_PEA_{—}1_P22 (SEQ ID NO:601) and Q9UJZ1 (SEQ ID NO:637):

1. An isolated chimeric polypeptide encoding for D11853_PEA_{—}1_P22 (SEQ ID NO:601), comprising a first amino acid sequence being at least 90% homologous to MLARAARGTGALLLRGSLLASGRAPRRASSGLPRNTVVLFVPQQEAWVVERMGRFHRILEP corresponding to amino acids 1-61 of Q9UJZ1 (SEQ ID NO:637), which also corresponds to amino acids 1-61 of D11853_PEA_{—}1_P22 (SEQ ID NO:601), and a second amino acid sequence being at least 70%, optionally at least 80%, preferably at least 85%, more preferably at least 90% and most preferably at least 95% homologous to a polypeptide having the sequence ELLLFWACSMC (SEQ ID NO:1555) corresponding to amino acids 62-72 of D11853_PEA_{—}1_P22 (SEQ ID NO:601), wherein said first amino acid sequence and second amino acid sequence are contiguous and in a sequential order.

2. An isolated polypeptide encoding for a tail of D11853_PEA_{—}1_P22 (SEQ ID NO:601), comprising a polypeptide being at least 70%, optionally at least about 80%, preferably at least about 85%, more preferably at least about 90% and most preferably at least about 95% homologous to the sequence ELLLFWACSMC (SEQ ID NO:1555) in D11853_PEA_{—}1_P22 (SEQ ID NO:601).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P22 (SEQ ID NO:601) also has the following non-silent SNPs (Single Nucleotide Polymorphisms) as listed in Table 32, (given according to their position(s) on the amino acid sequence, with the alternative amino acid(s) listed; the last column indicates whether the SNP is known or not; the presence of known SNPs in variant protein D11853_PEA_{—}1_P22 (SEQ ID NO:601) sequence provides support for the deduced sequence of this variant protein according to the present invention).

TABLE 32
Amino acid mutations

| SNP position(s) | | |
|---|---|---|
| on amino acid | Alternative | Previously |
| sequence | amino acid(s) | known SNP? |
| 25 | P -> | No |
| 32 | L -> | No |

Variant protein D11853_PEA_{—}1_P22 (SEQ ID NO:601) is encoded by the following transcript(s): D11853_PEA_{—}1_T9 (SEQ ID NO:61), for which the sequence(s) is/are given at the end of the application. The coding portion of transcript D11853_PEA_{—}1_T9 (SEQ ID NO:61) is shown in bold; this coding portion starts at position 108 and ends at position 323. The transcript also has the following SNPs as listed in Table 33 (given according to their position on the nucleotide sequence, with the alternative nucleic acid listed; the last column indicates whether the SNP is known or not; the presence of known SNPs in variant protein D11853_PEA_{—}1_P22 (SEQ ID NO:601) sequence provides support for the deduced sequence of this variant protein according to the present invention).

TABLE 33
Nucleic acid SNPs

| SNP position | | |
|---|---|---|
| on nucleotide | Alternative | Previously |
| sequence | nucleic acid | known SNP? |
| 180 | C -> | No |
| 182 | G -> | No |
| 185 | -> C | No |
| 201 | T -> | No |
| 757 | A -> C | No |
| 757 | A -> G | No |
| 758 | G -> | No |
| 758 | G -> A | No |
| 777 | C -> G | No |
| 784 | A -> C | No |
| 840 | G -> A | No |
| 912 | G -> A | No |
| 912 | G -> C | No |
| 929 | A -> | No |
| 929 | A -> C | No |
| 940 | A -> C | No |
| 955 | C -> | No |
| 955 | C -> G | No |
| 1025 | A -> C | No |
| 1049 | A -> C | No |
| 1057 | T -> G | No |
| 1075 | G -> A | No |
| 1075 | G -> C | No |
| 1083 | -> C | No |
| 1083 | -> T | No |
| 1090 | -> G | No |
| 1119 | C -> | No |
| 1119 | C -> G | No |
| 1150 | -> G | No |
| 1200 | G -> | No |
| 1209 | G -> A | No |
| 1229 | C -> T | No |
| 1244 | G -> | No |
| 1325 | G -> A | No |
| 1328 | A -> | No |
| 1328 | A -> C | No |
| 1374 | T -> | No |
| 1377 | T -> C | No |
| 1377 | T -> G | No |
| 1414 | T -> C | Yes |

Variant protein D11853_PEA_{—}1_P24 (SEQ ID NO:602) according to the present invention has an amino acid sequence as given at the end of the application; it is encoded by transcript(s) D11853_PEA_{—}1_T21 (SEQ ID NO:69). An alignment is given to the known protein (Membrane associated protein SLP-2 (SEQ ID NO:637)) at the end of the application. One or more alignments to one or more previously published protein sequences are given at the end of the application. A brief description of the relationship of the variant protein according to the present invention to each such aligned protein is as follows:

Comparison Report Between D11853_PEA_{—}1_P24 (SEQ ID NO:602) and Q9P042 (SEQ ID NO:639):

1. An isolated chimeric polypeptide encoding for D11853_PEA_{—}1_P24 (SEQ ID NO:602), comprising a first amino acid sequence being at least 70%, optionally at least 80%, preferably at least 85%, more preferably at least 90% and most preferably at least 95% homologous to a polypeptide having the sequence MLARAARGTGALLLRGSLLASGRAPR (SEQ ID NO:1530) corresponding to amino acids 1-26 of D11853_PEA_{—}1_P24 (SEQ ID NO:602), a second amino acid sequence being at least 90% homologous to RASSGLPRNTVVLFVPQQEAWVVERMGRFHRILEPGLNILIPVLDRIRYVQSLKEIVINVPEQSAVTL corresponding to amino acids 13-80 of Q9P042 (SEQ ID NO:639), which also corresponds to amino acids 27-94 of D11853_PEA_{—}1_P24 (SEQ ID NO:602), and a third amino acid sequence being at least 70%, optionally at least 80%, preferably at least 85%, more preferably at least 90% and most preferably at least 95% homologous to a polypeptide having the sequence GTGVPECQHCGCHQPSC (SEQ ID NO:1557) corresponding to amino acids 95-111 of D11853_PEA_{—}1_P24 (SEQ ID NO:602), wherein said first amino acid sequence, second amino acid sequence and third amino acid sequence are contiguous and in a sequential order.

2. An isolated polypeptide encoding for a head of D11853_PEA_{—}1_P24 (SEQ ID NO:602), comprising a polypeptide being at least 70%, optionally at least about 80%, preferably at least about 85%, more preferably at least about 90% and most preferably at least about 95% homologous to the sequence MLARAARGTGALLLRGSLLASGRAPR (SEQ ID NO:1530) of D11853_PEA_{—}1_P24 (SEQ ID NO:602).

3. An isolated polypeptide encoding for a tail of D11853_PEA_{—}1_P24 (SEQ ID NO:602), comprising a polypeptide being at least 70%, optionally at least about 80%, preferably at least about 85%, more preferably at least about 90% and most preferably at least about 95% homologous to the sequence GTGVPECQHCGCHQPSC (SEQ ID NO:1557) in D11853_PEA_{—}1_P24 (SEQ ID NO:602).

Comparison Report Between D11853_PEA_{—}1_P24 (SEQ ID NO:602) and Q96FY2 (SEQ ID NO:638):

1. An isolated chimeric polypeptide encoding for D11853_PEA_{—}1_P24 (SEQ ID NO:602), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTL corresponding to amino acids 1-94 of Q96FY2 (SEQ ID NO:638), which also corresponds to amino acids 1-94 of D11853_PEA_{—}1_P24 (SEQ ID NO:602), and a second amino acid sequence being at least 70%, optionally at least 80%, preferably at least 85%, more preferably at least 90% and most preferably at least 95% homologous to a polypeptide having the sequence GTGVPECQHCGCHQPSC (SEQ ID NO:1557) corresponding to amino acids 95-111 of D11853_PEA_{—}1_P24 (SEQ ID NO:602), wherein said first amino acid sequence and second amino acid sequence are contiguous and in a sequential order.

2. An isolated polypeptide encoding for a tail of D11853_PEA_{—}1_P24 (SEQ ID NO:602), comprising a polypeptide being at least 70%, optionally at least about 80%, preferably at least about 85%, more preferably at least about 90% and most preferably at least about 95% homologous to the sequence GTGVPECQHCGCHQPSC (SEQ ID NO:1557) in D11853_PEA_{—}1_P24 (SEQ ID NO:602).

Comparison Report Between D11853_PEA_{—}1_P24 (SEQ ID NO:602) and Q9UJZ1 (SEQ ID NO:637):

1. An isolated chimeric polypeptide encoding for D11853_PEA_{—}1_P24 (SEQ ID NO:602), comprising a first amino acid sequence being at least 90% homologous to MLARAARGTGALLLRGSLLASGRAPRRASSGLPRNTVVLFVPQQEAWVVERMGRFHRILEPGLNILIPVLD RIRYVQSLKEIVINVPEQSAVTL corresponding to amino acids 1-94 of Q9UJZ1 (SEQ ID NO:637), which also corresponds to amino acids 1-94 of D11853_PEA_{—}1_P24 (SEQ ID NO:602), and a second amino acid sequence being at least 70%, optionally at least 80%, preferably at least 85%, more preferably at least 90% and most preferably at least 95% homologous to a polypeptide having the sequence GTGVPECQHCGCHQPSC (SEQ ID NO:1557) corresponding to amino acids 95-111 of D11853_PEA_{—}1_P24 (SEQ ID NO:602), wherein said first amino acid sequence and second amino acid sequence are contiguous and in a sequential order.

2. An isolated polypeptide encoding for a tail of D11853_PEA_{—}1_P24 (SEQ ID NO:602), comprising a polypeptide being at least 70%, optionally at least about 80%, preferably at least about 85%, more preferably at least about 90% and most preferably at least about 95% homologous to the sequence GTGVPECQHCGCHQPSC (SEQ ID NO:1557) in D11853_PEA_{—}1_P24 (SEQ ID NO:602).

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein D11853_PEA_{—}1_P24 (SEQ ID NO:602) also has the following non-silent SNPs (Single Nucleotide Polymorphisms) as listed in Table 34, (given according to their position(s) on the amino acid sequence, with the alternative amino acid(s) listed; the last column indicates whether the SNP is known or not; the presence of known SNPs in variant protein D11853_PEA_{—}1_P24 (SEQ ID NO:602) sequence provides support for the deduced sequence of this variant protein according to the present invention).

TABLE 34
Amino acid mutations

| SNP position(s) | | |
|---|---|---|
| on amino acid | Alternative | Previously |
| sequence | amino acid(s) | known SNP? |
| 25 | P -> | No |
| 32 | L -> | No |

Variant protein D11853_PEA_{—}1_P24 (SEQ ID NO:602) is encoded by the following transcript(s): D11853_PEA_{—}1_T21 (SEQ ID NO:69), for which the sequence(s) is/are given at the end of the application. The coding portion of transcript D11853_PEA_{—}1_T21 (SEQ ID NO:69) is shown in bold; this coding portion starts at position 108 and ends at position 440. The transcript also has the following SNPs as listed in Table 35 (given according to their position on the nucleotide sequence, with the alternative nucleic acid listed; the last column indicates whether the SNP is known or not; the presence of known SNPs in variant protein D11853_PEA_{—}1_P24 (SEQ ID NO:602) sequence provides support for the deduced sequence of this variant protein according to the present invention).

TABLE 35
Nucleic acid SNPs

| SNP position | | |
|---|---|---|
| on nucleotide | Alternative | Previously |
| sequence | nucleic acid | known SNP? |
| 180 | C -> | No |
| 182 | G -> | No |
| 185 | -> C | No |
| 201 | T -> | No |
| 479 | A -> C | No |
| 479 | A -> G | No |
| 480 | G -> | No |
| 480 | G -> A | No |
| 499 | C -> G | No |
| 506 | A -> C | No |
| 562 | G -> A | No |
| 634 | G -> A | No |
| 634 | G -> C | No |
| 651 | A -> | No |
| 651 | A -> C | No |
| 662 | A -> C | No |
| 677 | C -> | No |
| 677 | C -> G | No |
| 747 | A -> C | No |
| 771 | A -> C | No |
| 779 | T -> G | No |
| 797 | G -> A | No |
| 797 | G -> C | No |
| 805 | -> C | No |
| 805 | -> T | No |
| 812 | -> G | No |
| 841 | C -> | No |
| 841 | C -> G | No |
| 872 | -> G | No |
| 922 | G -> | No |
| 931 | G -> A | No |
| 951 | C -> T | No |
| 966 | G -> | No |
| 1047 | G -> A | No |
| 1050 | A -> | No |
| 1050 | A -> C | No |
| 1096 | T -> | No |
| 1099 | T -> C | No |
| 1099 | T -> G | No |
| 1136 | T -> C | Yes |

As noted above, cluster D11853 features 31 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster D11853_PEA_{—}1_node_{—}3 (SEQ ID NO:419) according to the present invention is supported by 24 libraries. The number of libraries was determined as previously described. This segment can be found in the following transcript(s): D11853_PEA_{—}1_T7 (SEQ ID NO:59), D11853_PEA_{—}1_T17 (SEQ ID NO:67) and D11853_PEA_{—}1_T25 (SEQ ID NO:72). Table 36 below describes the starting and ending position of this segment on each transcript.

TABLE 36
Segment location on transcripts

| | | Segment |
|---|---|---|
| | Segment | ending |
| Transcript name | starting position | position |
| D11853_PEA_1_T7 (SEQ ID NO: 59) | 153 | 378 |
| D11853_PEA_1_T17 (SEQ ID NO: 67) | 153 | 378 |
| D11853_PEA_1_T25 (SEQ ID NO: 72) | 153 | 378 |

Segment cluster D11853_PEA_{—}1_node_{—}6 (SEQ ID NO:420) according to the present invention is supported by 25 libraries. The number of libraries was determined as previously described. This segment can be found in the following transcript(s): D11853_PEA_{—}1_T7 (SEQ ID NO:59), D11853_PEA_{—}1_T8 (SEQ ID NO:60), D11853_PEA_{—}1_T17 (SEQ ID NO:67) and D11853_PEA_{—}1_T25 (SEQ ID NO:72). Table 37 below describes the starting and ending position of this segment on each transcript.

TABLE 37
Segment location on transcripts

| | | Segment |
|---|---|---|
| | Segment | ending |
| Transcript name | starting position | position |
| D11853_PEA_1_T7 (SEQ ID NO: 59) | 517 | 890 |
| D11853_PEA_1_T8 (SEQ ID NO: 60) | 291 | 664 |
| D11853_PEA_1_T17 (SEQ ID NO: 67) | 517 | 890 |
| D11853_PEA_1_T25 (SEQ ID NO: 72) | 517 | 890 |

Segment cluster D11853_PEA_{—}1_node_{—}9 (SEQ ID NO:421) according to the present invention is supported by 1 libraries. The number of libraries was determined as previously described. This segment can be found in the following transcript(s): D11853_PEA_{—}1_T25 (SEQ ID NO:72). Table 38 below describes the starting and ending position of this segment on each transcript.

TABLE 38
Segment location on transcripts

| | | Segment |
|---|---|---|
| | Segment | ending |
| Transcript name | starting position | position |
| D11853_PEA_1_T25 (SEQ ID NO: 72) | 1108 | 1239 |

Segment cluster D11853_PEA_{—}1_node_{—}17 (SEQ ID NO:422) according to the present invention is supported by 3 libraries. The number of libraries was determined as previously described. This segment can be found in the following transcript(s): D11853_PEA_{—}1_T16 (SEQ ID NO:66) and D11853_PEA_{—}1_T23 (SEQ ID NO:70). Table 39 below describes the starting and ending position of this segment on each transcript.

TABLE 39
Segment location on transcripts

| | | Segment |
|---|---|---|
| | Segment | ending |
| Transcript name | starting position | position |
| D11853_PEA_1_T16 (SEQ ID NO: 66) | 552 | 700 |
| D11853_PEA_1_T23 (SEQ ID NO: 70) | 552 | 700 |

Segment cluster D11853_PEA_{—}1_node_{—}21 (SEQ ID NO:423) according to the present invention is supported by 6 libraries. The number of libraries was determined as previously described. This segment can be found in the following transcript(s): D11853_PEA_{—}1_T19 (SEQ ID NO:68) and D11853_PEA_{—}1_T23 (SEQ ID NO:70). Table 40 below describes the starting and ending position of this segment on each transcript.

TABLE 40
Segment location on transcripts

| | Segment | Segment |
|---|---|---|
| | starting | ending |
| Transcript name | position | position |
| D11853_PEA_1_T19 (SEQ ID NO: 68) | 687 | 830 |
| D11853_PEA_1_T23 (SEQ ID NO: 70) | 836 | 979 |

Segment cluster D11853_PEA_{—}1_node_{—}22 (SEQ ID NO:424) according to the present invention is supported by 287 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T17 (SEQ ID NO:59), D11853_PEA_{—}1_T8 (SEQ ID NO:60), D11853_PEA_{—}1_T19 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70), D11853_PEA_{—}1_T24 (SEQ ID NO:71) and D11853_PEA_{—}1_T25 (SEQ ID NO:72). Table 41 below describes the starting and ending position of this segment on each transcript.

TABLE 41
Segment location on transcripts

| | Segment | Segment |
|---|---|---|
| | starting | ending |
| Transcript name | position | position |
| D11853_PEA_1_T1 (SEQ ID NO: 57) | 687 | 831 |
| D11853_PEA_1_T3 (SEQ ID NO: 58) | 687 | 831 |
| D11853_PEA_1_T7 (SEQ ID NO: 59) | 1404 | 1548 |
| D11853_PEA_1_T8 (SEQ ID NO: 60) | 1178 | 1322 |
| D11853_PEA_1_T9 (SEQ ID NO: 61) | 804 | 948 |
| D11853_PEA_1_T10 (SEQ ID NO: 62) | 687 | 831 |
| D11853_PEA_1_T13 (SEQ ID NO: 63) | 687 | 831 |
| D11853_PEA_1_T14 (SEQ ID NO: 64) | 687 | 831 |
| D11853_PEA_1_T15 (SEQ ID NO: 65) | 687 | 831 |
| D11853_PEA_1_T16 (SEQ ID NO: 66) | 836 | 980 |
| D11853_PEA_1_T17 (SEQ ID NO: 67) | 1404 | 1548 |
| D11853_PEA_1_T19 (SEQ ID NO: 68) | 831 | 975 |
| D11853_PEA_1_T21 (SEQ ID NO: 69) | 526 | 670 |
| D11853_PEA_1_T23 (SEQ ID NO: 70) | 980 | 1124 |
| D11853_PEA_1_T24 (SEQ ID NO: 71) | 552 | 696 |
| D11853_PEA_1_T25 (SEQ ID NO: 72) | 1536 | 1680 |

Segment cluster D11853_PEA_{—}1_node_{—}23 (SEQ ID NO:425) according to the present invention is supported by 4 libraries. The number of libraries was determined as previously described. This segment can be found in the following transcript(s): D11853_PEA_{—}1_T13 (SEQ ID NO:63) and D11853_PEA_{—}1_T15 (SEQ ID NO:65). Table 42 below describes the starting and ending position of this segment on each transcript.

TABLE 42
Segment location on transcripts

| | Segment | Segment |
|---|---|---|
| | starting | ending |
| Transcript name | position | position |
| D11853_PEA_1_T13 (SEQ ID NO: 63) | 832 | 954 |
| D11853_PEA_1_T15 (SEQ ID NO: 65) | 832 | 954 |

Segment cluster D11853_PEA_{—}1_node_{—}25 (SEQ ID NO:426) according to the present invention is supported by 11 libraries. The number of libraries was determined as previously described. This segment can be found in the following transcript(s): D11853_PEA_{—}1_T10 (SEQ ID NO:62), D11853_PEA_{—}1_T15 (SEQ ID NO:65) and D11853_PEA_{—}1_T25 (SEQ ID NO:72). Table 43 below describes the starting and ending position of this segment on each transcript.

TABLE 43
Segment location on transcripts

| | Segment | Segment |
|---|---|---|
| | starting | ending |
| Transcript name | position | position |
| D11853_PEA_1_T10 (SEQ ID NO: 62) | 912 | 1116 |
| D11853_PEA_1_T15 (SEQ ID NO: 65) | 1035 | 1239 |
| D11853_PEA_1_T25 (SEQ ID NO: 72) | 1761 | 1965 |

Segment cluster D11853_PEA_{—}1_node_{—}26 (SEQ ID NO:427) according to the present invention is supported by 290 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70), D11853_PEA_{—}1_T24 (SEQ ID NO:71) and D11853_PEA_{—}1_T25 (SEQ ID NO:72). Table 44 below describes the starting and ending position of this segment on each transcript.

TABLE 44
Segment location on transcripts

| | Segment | Segment |
|---|---|---|
| | starting | ending |
| Transcript name | position | position |
| D11853_PEA_1_T1 (SEQ ID NO: 57) | 912 | 1040 |
| D11853_PEA_1_T3 (SEQ ID NO: 58) | 912 | 1040 |
| D11853_PEA_1_T7 (SEQ ID NO: 59) | 1629 | 1757 |
| D11853_PEA_1_T8 (SEQ ID NO: 60) | 1403 | 1531 |
| D11853_PEA_1_T9 (SEQ ID NO: 61) | 1029 | 1157 |
| D11853_PEA_1_T10 (SEQ ID NO: 62) | 1117 | 1245 |
| D11853_PEA_1_T13 (SEQ ID NO: 63) | 1035 | 1163 |
| D11853_PEA_1_T15 (SEQ ID NO: 65) | 1240 | 1368 |
| D11853_PEA_1_T16 (SEQ ID NO: 66) | 1061 | 1189 |
| D11853_PEA_1_T17 (SEQ ID NO: 67) | 1629 | 1757 |
| D11853_PEA_1_T19 (SEQ ID NO: 68) | 1056 | 1184 |
| D11853_PEA_1_T21 (SEQ ID NO: 69) | 751 | 879 |
| D11853_PEA_1_T23 (SEQ ID NO: 70) | 1205 | 1333 |
| D11853_PEA_1_T24 (SEQ ID NO: 71) | 777 | 905 |
| D11853_PEA_1_T25 (SEQ ID NO: 72) | 1966 | 2094 |

Segment cluster D11853_PEA_{—}1_node_{—}27 (SEQ ID NO:428) according to the present invention is supported by 8 libraries. The number of libraries was determined as previously described. This segment can be found in the following transcript(s): D11853_PEA_{—}1_T3 (SEQ ID NO:58) and D11853_PEA_{—}1_T25 (SEQ ID NO:72). Table 45 below describes the starting and ending position of this segment on each transcript.

TABLE 45
Segment location on transcripts

| | Segment | Segment |
|---|---|---|
| | starting | ending |
| Transcript name | position | position |
| D11853_PEA_1_T3 (SEQ ID NO: 58) | 1041 | 1460 |
| D11853_PEA_1_T25 (SEQ ID NO: 72) | 2095 | 2514 |

Segment cluster D11853_PEA_{—}1_node_{—}30 (SEQ ID NO:429) according to the present invention is supported by 249 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70), D11853_PEA_{—}1_T24 (SEQ ID NO:71), D11853_PEA_{—}1_T25 (SEQ ID NO:72), D11853_PEA_{—}1_T26 (SEQ ID NO:73) and D11853_PEA_{—}1_T27 (SEQ ID NO:74). Table 46 below describes the starting and ending position of this segment on each transcript.

TABLE 46
Segment location on transcripts

| | Segment | Segment |
|---|---|---|
| | starting | ending |
| Transcript name | position | position |
| D11853_PEA_1_T1 (SEQ ID NO: 57) | 1088 | 1207 |
| D11853_PEA_1_T3 (SEQ ID NO: 58) | 1513 | 1632 |
| D11853_PEA_1_T7 (SEQ ID NO: 59) | 1805 | 1924 |
| D11853_PEA_1_T8 (SEQ ID NO: 60) | 1579 | 1698 |
| D11853_PEA_1_T9 (SEQ ID NO: 61) | 1205 | 1324 |
| D11853_PEA_1_T10 (SEQ ID NO: 62) | 1293 | 1412 |
| D11853_PEA_1_T13 (SEQ ID NO: 63) | 1211 | 1330 |
| D11853_PEA_1_T14 (SEQ ID NO: 64) | 959 | 1078 |
| D11853_PEA_1_T15 (SEQ ID NO: 65) | 1416 | 1535 |
| D11853_PEA_1_T16 (SEQ ID NO: 66) | 1237 | 1356 |
| D11853_PEA_1_T17 (SEQ ID NO: 67) | 1805 | 1924 |
| D11853_PEA_1_T19 (SEQ ID NO: 68) | 1232 | 1351 |
| D11853_PEA_1_T21 (SEQ ID NO: 69) | 927 | 1046 |
| D11853_PEA_1_T23 (SEQ ID NO: 70) | 1381 | 1500 |
| D11853_PEA_1_T24 (SEQ ID NO: 71) | 953 | 1072 |
| D11853_PEA_1_T25 (SEQ ID NO: 72) | 2567 | 2686 |
| D11853_PEA_1_T26 (SEQ ID NO: 73) | 635 | 754 |
| D11853_PEA_1_T27 (SEQ ID NO: 74) | 534 | 653 |

Segment cluster D11853_PEA_{—}1_node_{—}32 (SEQ ID NO:430) according to the present invention is supported by 215 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70), D11853_PEA_{—}1_T24 (SEQ ID NO:71), D11853_PEA_{—}1_T25 (SEQ ID NO:72), D11853_PEA_{—}1_T26 (SEQ ID NO:73) and D11853_PEA_{—}1_T27 (SEQ ID NO:74). Table 47 below describes the starting and ending position of this segment on each transcript.

TABLE 47
Segment location on transcripts

| | Segment | Segment |
|---|---|---|
| | starting | ending |
| Transcript name | position | position |
| D11853_PEA_1_T1 (SEQ ID NO: 57) | 1208 | 1437 |
| D11853_PEA_1_T3 (SEQ ID NO: 58) | 1633 | 1745 |
| D11853_PEA_1_T7 (SEQ ID NO: 59) | 1925 | 2037 |
| D11853_PEA_1_T8 (SEQ ID NO: 60) | 1699 | 1811 |
| D11853_PEA_1_T9 (SEQ ID NO: 61) | 1325 | 1437 |
| D11853_PEA_1_T10 (SEQ ID NO: 62) | 1413 | 1525 |
| D11853_PEA_1_T13 (SEQ ID NO: 63) | 1331 | 1443 |
| D11853_PEA_1_T14 (SEQ ID NO: 64) | 1079 | 1191 |
| D11853_PEA_1_T15 (SEQ ID NO: 65) | 1536 | 1648 |
| D11853_PEA_1_T16 (SEQ ID NO: 66) | 1357 | 1469 |
| D11853_PEA_1_T17 (SEQ ID NO: 67) | 1925 | 2154 |
| D11853_PEA_1_T19 (SEQ ID NO: 68) | 1352 | 1464 |
| D11853_PEA_1_T21 (SEQ ID NO: 69) | 1047 | 1159 |
| D11853_PEA_1_T23 (SEQ ID NO: 70) | 1501 | 1613 |
| D11853_PEA_1_T24 (SEQ ID NO: 71) | 1073 | 1185 |
| D11853_PEA_1_T25 (SEQ ID NO: 72) | 2687 | 2799 |
| D11853_PEA_1_T26 (SEQ ID NO: 73) | 755 | 984 |
| D11853_PEA_1_T27 (SEQ ID NO: 74) | 654 | 766 |

According to an optional embodiment of the present invention, short segments related to the above cluster are also provided. These segments are up to about 120 bp in length, and so are included in a separate description.

Segment cluster D11853_PEA_{—}1_node_{—}0 (SEQ ID NO:431) according to the present invention is supported by 14 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70), D11853_PEA_{—}1_T24 (SEQ ID NO:71), D11853_PEA_{—}1_T25 (SEQ ID NO:72), D11853_PEA_{—}1_T26 (SEQ ID NO:73) and D11853_PEA_{—}1_T27 (SEQ ID NO:74). Table 48 below describes the starting and ending position of this segment on each transcript.

TABLE 48
Segment location on transcripts

| | Segment | Segment |
|---|---|---|
| | starting | ending |
| Transcript name | position | position |
| D11853_PEA_1_T1 (SEQ ID NO: 57) | 1 | 41 |
| D11853_PEA_1_T3 (SEQ ID NO: 58) | 1 | 41 |
| D11853_PEA_1_T7 (SEQ ID NO: 59) | 1 | 41 |
| D11853_PEA_1_T8 (SEQ ID NO: 60) | 1 | 41 |
| D11853_PEA_1_T9 (SEQ ID NO: 61) | 1 | 41 |
| D11853_PEA_1_T10 (SEQ ID NO: 62) | 1 | 41 |
| D11853_PEA_1_T13 (SEQ ID NO: 63) | 1 | 41 |
| D11853_PEA_1_T14 (SEQ ID NO: 64) | 1 | 41 |
| D11853_PEA_1_T15 (SEQ ID NO: 65) | 1 | 41 |
| D11853_PEA_1_T16 (SEQ ID NO: 66) | 1 | 41 |
| D11853_PEA_1_T17 (SEQ ID NO: 67) | 1 | 41 |
| D11853_PEA_1_T19 (SEQ ID NO: 68) | 1 | 41 |
| D11853_PEA_1_T21 (SEQ ID NO: 69) | 1 | 41 |
| D11853_PEA_1_T23 (SEQ ID NO: 70) | 1 | 41 |
| D11853_PEA_1_T24 (SEQ ID NO: 71) | 1 | 41 |
| D11853_PEA_1_T25 (SEQ ID NO: 72) | 1 | 41 |
| D11853_PEA_1_T26 (SEQ ID NO: 73) | 1 | 41 |
| D11853_PEA_1_T27 (SEQ ID NO: 74) | 1 | 41 |

Segment cluster D11853_PEA_{—}1_node_{—}1 (SEQ ID NO:432) according to the present invention is supported by 158 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70), D11853_PEA_{—}1_T24 (SEQ ID NO:71), D11853_PEA_{—}1_T25 (SEQ ID NO:72), D11853_PEA_{—}1_T26 (SEQ ID NO:73) and D11853_PEA_{—}1_T27 (SEQ ID NO:74). Table 49 below describes the starting and ending position of this segment on each transcript.

TABLE 49
Segment location on transcripts

| | Segment | Segment |
|---|---|---|
| | starting | ending |
| Transcript name | position | position |
| D11853_PEA_1_T1 (SEQ ID NO: 57) | 42 | 110 |
| D11853_PEA_1_T3 (SEQ ID NO: 58) | 42 | 110 |
| D11853_PEA_1_T7 (SEQ ID NO: 59) | 42 | 110 |
| D11853_PEA_1_T8 (SEQ ID NO: 60) | 42 | 110 |
| D11853_PEA_1_T9 (SEQ ID NO: 61) | 42 | 110 |
| D11853_PEA_1_T10 (SEQ ID NO: 62) | 42 | 110 |
| D11853_PEA_1_T13 (SEQ ID NO: 63) | 42 | 110 |
| D11853_PEA_1_T14 (SEQ ID NO: 64) | 42 | 110 |
| D11853_PEA_1_T15 (SEQ ID NO: 65) | 42 | 110 |
| D11853_PEA_1_T16 (SEQ ID NO: 66) | 42 | 110 |
| D11853_PEA_1_T17 (SEQ ID NO: 67) | 42 | 110 |
| D11853_PEA_1_T19 (SEQ ID NO: 68) | 42 | 110 |
| D11853_PEA_1_T21 (SEQ ID NO: 69) | 42 | 110 |
| D11853_PEA_1_T23 (SEQ ID NO: 70) | 42 | 110 |
| D11853_PEA_1_T24 (SEQ ID NO: 71) | 42 | 110 |
| D11853_PEA_1_T25 (SEQ ID NO: 72) | 42 | 110 |
| D11853_PEA_1_T26 (SEQ ID NO: 73) | 42 | 110 |
| D11853_PEA_1_T27 (SEQ ID NO: 74) | 42 | 110 |

Segment cluster D11853_PEA_{—}1_node_{—}2 (SEQ ID NO:433) according to the present invention is supported by 247 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70), D11853_PEA_{—}1_T24 (SEQ ID NO:71), D11853_PEA_{—}1_T25 (SEQ ID NO:72), D11853_PEA_{—}1_T26 (SEQ ID NO:73) and D11853_PEA_{—}1_T27 (SEQ ID NO:74). Table 50 below describes the starting and ending position of this segment on each transcript.

TABLE 50
Segment location on transcripts

| | Segment | Segment |
|---|---|---|
| | starting | ending |
| Transcript name | position | position |
| D11853_PEA_1_T1 (SEQ ID NO: 57) | 111 | 152 |
| D11853_PEA_1_T3 (SEQ ID NO: 58) | 111 | 152 |
| D11853_PEA_1_T7 (SEQ ID NO: 59) | 111 | 152 |
| D11853_PEA_1_T8 (SEQ ID NO: 60) | 111 | 152 |
| D11853_PEA_1_T9 (SEQ ID NO: 61) | 111 | 152 |
| D11853_PEA_1_T10 (SEQ ID NO: 62) | 111 | 152 |
| D11853_PEA_1_T13 (SEQ ID NO: 63) | 111 | 152 |
| D11853_PEA_1_T14 (SEQ ID NO: 64) | 111 | 152 |
| D11853_PEA_1_T15 (SEQ ID NO: 65) | 111 | 152 |
| D11853_PEA_1_T16 (SEQ ID NO: 66) | 111 | 152 |
| D11853_PEA_1_T17 (SEQ ID NO: 67) | 111 | 152 |
| D11853_PEA_1_T19 (SEQ ID NO: 68) | 111 | 152 |
| D11853_PEA_1_T21 (SEQ ID NO: 69) | 111 | 152 |
| D11853_PEA_1_T23 (SEQ ID NO: 70) | 111 | 152 |
| D11853_PEA_1_T24 (SEQ ID NO: 71) | 111 | 152 |
| D11853_PEA_1_T25 (SEQ ID NO: 72) | 111 | 152 |
| D11853_PEA_1_T26 (SEQ ID NO: 73) | 111 | 152 |
| D11853_PEA_1_T27 (SEQ ID NO: 74) | 111 | 152 |

Segment cluster D11853_PEA_{—}1_node_{—}4 (SEQ ID NO:434) according to the present invention is supported by 258 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70), D11853_PEA_{—}1_T24 (SEQ ID NO:71), D11853_PEA_{—}1_T25 (SEQ ID NO:72), D11853_PEA_{—}1_T26 (SEQ ID NO:73) and D11853_PEA_{—}1_T27 (SEQ ID NO:74). Table 51 below describes the starting and ending position of this segment on each transcript.

TABLE 51
Segment location on transcripts

| | Segment | Segment |
|---|---|---|
| | starting | ending |
| Transcript name | position | position |
| D11853_PEA_1_T1 (SEQ ID NO: 57) | 153 | 185 |
| D11853_PEA_1_T3 (SEQ ID NO: 58) | 153 | 185 |
| D11853_PEA_1_T7 (SEQ ID NO: 59) | 379 | 411 |
| D11853_PEA_1_T8 (SEQ ID NO: 60) | 153 | 185 |
| D11853_PEA_1_T9 (SEQ ID NO: 61) | 153 | 185 |
| D11853_PEA_1_T10 (SEQ ID NO: 62) | 153 | 185 |
| D11853_PEA_1_T13 (SEQ ID NO: 63) | 153 | 185 |
| D11853_PEA_1_T14 (SEQ ID NO: 64) | 153 | 185 |
| D11853_PEA_1_T15 (SEQ ID NO: 65) | 153 | 185 |
| D11853_PEA_1_T16 (SEQ ID NO: 66) | 153 | 185 |
| D11853_PEA_1_T17 (SEQ ID NO: 67) | 379 | 411 |
| D11853_PEA_1_T19 (SEQ ID NO: 68) | 153 | 185 |
| D11853_PEA_1_T21 (SEQ ID NO: 69) | 153 | 185 |
| D11853_PEA_1_T23 (SEQ ID NO: 70) | 153 | 185 |
| D11853_PEA_1_T24 (SEQ ID NO: 71) | 153 | 185 |
| D11853_PEA_1_T25 (SEQ ID NO: 72) | 379 | 411 |
| D11853_PEA_1_T26 (SEQ ID NO: 73) | 153 | 185 |
| D11853_PEA_1_T27 (SEQ ID NO: 74) | 153 | 185 |

Segment cluster D11853_PEA_{—}1_node_{—}5 (SEQ ID NO:435) according to the present invention is supported by 291 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70), D11853_PEA_{—}1_T24 (SEQ ID NO:71), D11853_PEA_{—}1_T25 (SEQ ID NO:72), D11853_PEA_{—}1_T26 (SEQ ID NO:73) and D11853_PEA_{—}1_T27 (SEQ ID NO:74). Table 52 below describes the starting and ending position of this segment on each transcript.

TABLE 52
Segment location on transcripts

| | Segment | Segment |
|---|---|---|
| | starting | ending |
| Transcript name | position | position |
| D11853_PEA_1_T1 (SEQ ID NO: 57) | 186 | 290 |
| D11853_PEA_1_T3 (SEQ ID NO: 58) | 186 | 290 |
| D11853_PEA_1_T7 (SEQ ID NO: 59) | 412 | 516 |
| D11853_PEA_1_T8 (SEQ ID NO: 60) | 186 | 290 |
| D11853_PEA_1_T9 (SEQ ID NO: 61) | 186 | 290 |
| D11853_PEA_1_T10 (SEQ ID NO: 62) | 186 | 290 |
| D11853_PEA_1_T13 (SEQ ID NO: 63) | 186 | 290 |
| D11853_PEA_1_T14 (SEQ ID NO: 64) | 186 | 290 |
| D11853_PEA_1_T15 (SEQ ID NO: 65) | 186 | 290 |
| D11853_PEA_1_T16 (SEQ ID NO: 66) | 186 | 290 |
| D11853_PEA_1_T17 (SEQ ID NO: 67) | 412 | 516 |
| D11853_PEA_1_T19 (SEQ ID NO: 68) | 186 | 290 |
| D11853_PEA_1_T21 (SEQ ID NO: 69) | 186 | 290 |
| D11853_PEA_1_T23 (SEQ ID NO: 70) | 186 | 290 |
| D11853_PEA_1_T24 (SEQ ID NO: 71) | 186 | 290 |
| D11853_PEA_1_T25 (SEQ ID NO: 72) | 412 | 516 |
| D11853_PEA_1_T26 (SEQ ID NO: 73) | 186 | 290 |
| D11853_PEA_1_T27 (SEQ ID NO: 74) | 186 | 290 |

Segment cluster D11853_PEA_{—}1_node_{—}7 (SEQ ID NO:436) according to the present invention is supported by 20 libraries. The number of libraries was determined as previously described. This segment can be found in the following transcript(s): D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T17 (SEQ ID NO:67) and D11853_PEA_{—}1_T25 (SEQ ID NO:72). Table 53 below describes the starting and ending position of this segment on each transcript.

TABLE 53
Segment location on transcripts

| | Segment | Segment |
|---|---|---|
| | starting | ending |
| Transcript name | position | position |
| D11853_PEA_1_T7 (SEQ ID NO: 59) | 891 | 1007 |
| D11853_PEA_1_T8 (SEQ ID NO: 60) | 665 | 781 |
| D11853_PEA_1_T9 (SEQ ID NO: 61) | 291 | 407 |
| D11853_PEA_1_T17 (SEQ ID NO: 67) | 891 | 1007 |
| D11853_PEA_1_T25 (SEQ ID NO: 72) | 891 | 1007 |

Segment cluster D11853_PEA_{—}1_node_{—}8 (SEQ ID NO:437) according to the present invention is supported by 304 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70), D11853_PEA_{—}1_T24 (SEQ ID NO:71), D11853_PEA_{—}1_T25 (SEQ ID NO:72), D11853_PEA_{—}1_T26 (SEQ ID NO:73) and D11853_PEA_{—}1_T27 (SEQ ID NO:74). Table 54 below describes the starting and ending position of this segment on each transcript.

TABLE 54
Segment location on transcripts

| | Segment | Segment |
|---|---|---|
| | starting | ending |
| Transcript name | position | position |
| D11853_PEA_1_T1 (SEQ ID NO: 57) | 291 | 390 |
| D11853_PEA_1_T3 (SEQ ID NO: 58) | 291 | 390 |
| D11853_PEA_1_T7 (SEQ ID NO: 59) | 1008 | 1107 |
| D11853_PEA_1_T8 (SEQ ID NO: 60) | 782 | 881 |
| D11853_PEA_1_T9 (SEQ ID NO: 61) | 408 | 507 |
| D11853_PEA_1_T10 (SEQ ID NO: 62) | 291 | 390 |
| D11853_PEA_1_T13 (SEQ ID NO: 63) | 291 | 390 |
| D11853_PEA_1_T14 (SEQ ID NO: 64) | 291 | 390 |
| D11853_PEA_1_T15 (SEQ ID NO: 65) | 291 | 390 |
| D11853_PEA_1_T16 (SEQ ID NO: 66) | 291 | 390 |
| D11853_PEA_1_T17 (SEQ ID NO: 67) | 1008 | 1107 |
| D11853_PEA_1_T19 (SEQ ID NO: 68) | 291 | 390 |
| D11853_PEA_1_T21 (SEQ ID NO: 69) | 291 | 390 |
| D11853_PEA_1_T23 (SEQ ID NO: 70) | 291 | 390 |
| D11853_PEA_1_T24 (SEQ ID NO: 71) | 291 | 390 |
| D11853_PEA_1_T25 (SEQ ID NO: 72) | 1008 | 1107 |
| D11853_PEA_1_T26 (SEQ ID NO: 73) | 291 | 390 |
| D11853_PEA_1_T27 (SEQ ID NO: 74) | 291 | 390 |

Segment cluster D11853_PEA_{—}1_node_{—}10 (SEQ ID NO:438) according to the present invention is supported by 237 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T23 (SEQ ID NO:70), D11853_PEA_{—}1_T24 (SEQ ID NO:71), D11853_PEA_{—}1_T25 (SEQ ID NO:72), D11853_PEA_{—}1_T26 (SEQ ID NO:73) and D11853_PEA_{—}1_T27 (SEQ ID NO:74). Table 55 below describes the starting and ending position of this segment on each transcript.

TABLE 55
Segment location on transcripts

| | | Segment |
|---|---|---|
| | Segment | ending |
| Transcript name | starting position | position |
| D11853_PEA_1_T1 (SEQ ID NO: 57) | 391 | 449 |
| D11853_PEA_1_T3 (SEQ ID NO: 58) | 391 | 449 |
| D11853_PEA_1_T7 (SEQ ID NO: 59) | 1108 | 1166 |
| D11853_PEA_1_T8 (SEQ ID NO: 60) | 882 | 940 |
| D11853_PEA_1_T9 (SEQ ID NO: 61) | 508 | 566 |
| D11853_PEA_1_T10 (SEQ ID NO: 62) | 391 | 449 |
| D11853_PEA_1_T13 (SEQ ID NO: 63) | 391 | 449 |
| D11853_PEA_1_T14 (SEQ ID NO: 64) | 391 | 449 |
| D11853_PEA_1_T15 (SEQ ID NO: 65) | 391 | 449 |
| D11853_PEA_1_T16 (SEQ ID NO: 66) | 391 | 449 |
| D11853_PEA_1_T17 (SEQ ID NO: 67) | 1108 | 1166 |
| D11853_PEA_1_T19 (SEQ ID NO: 68) | 391 | 449 |
| D11853_PEA_1_T23 (SEQ ID NO: 70) | 391 | 449 |
| D11853_PEA_1_T24 (SEQ ID NO: 71) | 391 | 449 |
| D11853_PEA_1_T25 (SEQ ID NO: 72) | 1240 | 1298 |
| D11853_PEA_1_T26 (SEQ ID NO: 73) | 391 | 449 |
| D11853_PEA_1_T27 (SEQ ID NO: 74) | 391 | 449 |

Segment cluster D11853_PEA_{—}1_node_{—}12 (SEQ ID NO:439) according to the present invention is supported by 239 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T23 (SEQ ID NO:70), D11853_PEA_{—}1_T24 (SEQ ID NO:71), D11853_PEA_{—}1_T25 (SEQ ID NO:72), D11853_PEA_{—}1_T26 (SEQ ID NO:73) and D11853_PEA_{—}1_T27 (SEQ ID NO:74). Table 56 below describes the starting and ending position of this segment on each transcript.

TABLE 56
Segment location on transcripts

| | | Segment |
|---|---|---|
| | Segment | ending |
| Transcript name | starting position | position |
| D11853_PEA_1_T1 (SEQ ID NO: 57) | 450 | 482 |
| D11853_PEA_1_T3 (SEQ ID NO: 58) | 450 | 482 |
| D11853_PEA_1_T7 (SEQ ID NO: 59) | 1167 | 1199 |
| D11853_PEA_1_T8 (SEQ ID NO: 60) | 941 | 973 |
| D11853_PEA_1_T9 (SEQ ID NO: 61) | 567 | 599 |
| D11853_PEA_1_T10 (SEQ ID NO: 62) | 450 | 482 |
| D11853_PEA_1_T13 (SEQ ID NO: 63) | 450 | 482 |
| D11853_PEA_1_T14 (SEQ ID NO: 64) | 450 | 482 |
| D11853_PEA_1_T15 (SEQ ID NO: 65) | 450 | 482 |
| D11853_PEA_1_T16 (SEQ ID NO: 66) | 450 | 482 |
| D11853_PEA_1_T17 (SEQ ID NO: 67) | 1167 | 1199 |
| D11853_PEA_1_T19 (SEQ ID NO: 68) | 450 | 482 |
| D11853_PEA_1_T23 (SEQ ID NO: 70) | 450 | 482 |
| D11853_PEA_1_T24 (SEQ ID NO: 71) | 450 | 482 |
| D11853_PEA_1_T25 (SEQ ID NO: 72) | 1299 | 1331 |
| D11853_PEA_1_T26 (SEQ ID NO: 73) | 450 | 482 |
| D11853_PEA_1_T27 (SEQ ID NO: 74) | 450 | 482 |

Segment cluster D11853_PEA_{—}1_node_{—}13 (SEQ ID NO:440) according to the present invention can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62) D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T23 (SEQ ID NO:70), D11853_PEA_{—}1_T24 (SEQ ID NO:71), D11853_PEA_{—}1_T25 (SEQ ID NO:72), D11853_PEA_{—}1_T26 (SEQ ID NO:73) and D11853_PEA_{—}1_T27 (SEQ ID NO:74). Table 57 below describes the starting and ending position of this segment on each transcript.

TABLE 57
Segment location on transcripts

| | | Segment |
|---|---|---|
| | Segment | ending |
| Transcript name | starting position | position |
| D11853_PEA_1_T1 (SEQ ID NO: 57) | 483 | 495 |
| D11853_PEA_1_T3 (SEQ ID NO: 58) | 483 | 495 |
| D11853_PEA_1_T7 (SEQ ID NO: 59) | 1200 | 1212 |
| D11853_PEA_1_T8 (SEQ ID NO: 60) | 974 | 986 |
| D11853_PEA_1_T9 (SEQ ID NO: 61) | 600 | 612 |
| D11853_PEA_1_T10 (SEQ ID NO: 62) | 483 | 495 |
| D11853_PEA_1_T13 (SEQ ID NO: 63) | 483 | 495 |
| D11853_PEA_1_T14 (SEQ ID NO: 64) | 483 | 495 |
| D11853_PEA_1_T15 (SEQ ID NO: 65) | 483 | 495 |
| D11853_PEA_1_T16 (SEQ ID NO: 66) | 483 | 495 |
| D11853_PEA_1_T17 (SEQ ID NO: 67) | 1200 | 1212 |
| D11853_PEA_1_T19 (SEQ ID NO: 68) | 483 | 495 |
| D11853_PEA_1_T23 (SEQ ID NO: 70) | 483 | 495 |
| D11853_PEA_1_T24 (SEQ ID NO: 71) | 483 | 495 |
| D11853_PEA_1_T25 (SEQ ID NO: 72) | 1332 | 1344 |
| D11853_PEA_1_T26 (SEQ ID NO: 73) | 483 | 495 |
| D11853_PEA_1_T27 (SEQ ID NO: 74) | 483 | 495 |

Segment cluster D11853_PEA_{—}1_node_{—}14 (SEQ ID NO:441) according to the present invention can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T23 (SEQ ID NO:70), D11853_PEA_{—}1_T24 (SEQ ID NO:71), D11853_PEA_{—}1_T25 (SEQ ID NO:72), D11853_PEA_{—}1_T26 (SEQ ID NO:73) and D11853_PEA_{—}1_T27 (SEQ ID NO:74). Table 58 below describes the starting and ending position of this segment on each transcript.

TABLE 58
Segment location on transcripts

| | | Segment |
|---|---|---|
| | Segment | ending |
| Transcript name | starting position | position |
| D11853_PEA_1_T1 (SEQ ID NO: 57) | 496 | 511 |
| D11853_PEA_1_T3 (SEQ ID NO: 58) | 496 | 511 |
| D11853_PEA_1_T7 (SEQ ID NO: 59) | 1213 | 1228 |
| D11853_PEA_1_T8 (SEQ ID NO: 60) | 987 | 1002 |
| D11853_PEA_1_T9 (SEQ ID NO: 61) | 613 | 628 |
| D11853_PEA_1_T10 (SEQ ID NO: 62) | 496 | 511 |
| D11853_PEA_1_T13 (SEQ ID NO: 63) | 496 | 511 |
| D11853_PEA_1_T14 (SEQ ID NO: 64) | 496 | 511 |
| D11853_PEA_1_T15 (SEQ ID NO: 65) | 496 | 511 |
| D11853_PEA_1_T16 (SEQ ID NO: 66) | 496 | 511 |
| D11853_PEA_1_T17 (SEQ ID NO: 67) | 1213 | 1228 |
| D11853_PEA_1_T19 (SEQ ID NO: 68) | 496 | 511 |
| D11853_PEA_1_T23 (SEQ ID NO: 70) | 496 | 511 |
| D11853_PEA_1_T24 (SEQ ID NO: 71) | 496 | 511 |
| D11853_PEA_1_T25 (SEQ ID NO: 72) | 1345 | 1360 |
| D11853_PEA_1_T26 (SEQ ID NO: 73) | 496 | 511 |
| D11853_PEA_1_T27 (SEQ ID NO: 74) | 496 | 511 |

Segment cluster D11853_PEA_{—}1_node_{—}15 (SEQ ID NO:442) according to the present invention can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T23 (SEQ ID NO:70), D11853_PEA_{—}1_T24 (SEQ ID NO:71), D11853_PEA_{—}1_T25 (SEQ ID NO:72), D11853_PEA_{—}1_T26 (SEQ ID NO:73) and D11853_PEA_{—}1_T27 (SEQ ID NO:74). Table 59 below describes the starting and ending position of this segment on each transcript.

TABLE 59
Segment location on transcripts

| | | Segment |
|---|---|---|
| | Segment | ending |
| Transcript name | starting position | position |
| D11853_PEA_1_T1 (SEQ ID NO: 57) | 512 | 533 |
| D11853_PEA_1_T3 (SEQ ID NO: 58) | 512 | 533 |
| D11853_PEA_1_T7 (SEQ ID NO: 59) | 1229 | 1250 |
| D11853_PEA_1_T8 (SEQ ID NO: 60) | 1003 | 1024 |
| D11853_PEA_1_T9 (SEQ ID NO: 61) | 629 | 650 |
| D11853_PEA_1_T10 (SEQ ID NO: 62) | 512 | 533 |
| D11853_PEA_1_T13 (SEQ ID NO: 63) | 512 | 533 |
| D11853_PEA_1_T14 (SEQ ID NO: 64) | 512 | 533 |
| D11853_PEA_1_T15 (SEQ ID NO: 65) | 512 | 533 |
| D11853_PEA_1_T16 (SEQ ID NO: 66) | 512 | 533 |
| D11853_PEA_1_T17 (SEQ ID NO: 67) | 1229 | 1250 |
| D11853_PEA_1_T19 (SEQ ID NO: 68) | 512 | 533 |
| D11853_PEA_1_T23 (SEQ ID NO: 70) | 512 | 533 |
| D11853_PEA_1_T24 (SEQ ID NO: 71) | 512 | 533 |
| D11853_PEA_1_T25 (SEQ ID NO: 72) | 1361 | 1382 |
| D11853_PEA_1_T26 (SEQ ID NO: 73) | 512 | 533 |
| D11853_PEA_1_T27 (SEQ ID NO: 74) | 512 | 533 |

Segment cluster D11853_PEA_{—}1_node_{—}16 (SEQ ID NO:443) according to the present invention can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T23 (SEQ ID NO:70), D11853_PEA_{—}1_T24 (SEQ ID NO:71), D11853_PEA_{—}1_T25 (SEQ ID NO:72) and D11853_PEA_{—}1_T26 (SEQ ID NO:73). Table 60 below describes the starting and ending position of this segment on each transcript.

TABLE 60
Segment location on transcripts

| | | Segment |
|---|---|---|
| | Segment | ending |
| Transcript name | starting position | position |
| D11853_PEA_1_T1 (SEQ ID NO: 57) | 534 | 551 |
| D11853_PEA_1_T3 (SEQ ID NO: 58) | 534 | 551 |
| D11853_PEA_1_T7 (SEQ ID NO: 59) | 1251 | 1268 |
| D11853_PEA_1_T8 (SEQ ID NO: 60) | 1025 | 1042 |
| D11853_PEA_1_T9 (SEQ ID NO: 61) | 651 | 668 |
| D11853_PEA_1_T10 (SEQ ID NO: 62) | 534 | 551 |
| D11853_PEA_1_T13 (SEQ ID NO: 63) | 534 | 551 |
| D11853_PEA_1_T14 (SEQ ID NO: 64) | 534 | 551 |
| D11853_PEA_1_T15 (SEQ ID NO: 65) | 534 | 551 |
| D11853_PEA_1_T16 (SEQ ID NO: 66) | 534 | 551 |
| D11853_PEA_1_T17 (SEQ ID NO: 67) | 1251 | 1268 |
| D11853_PEA_1_T19 (SEQ ID NO: 68) | 534 | 551 |
| D11853_PEA_1_T23 (SEQ ID NO: 70) | 534 | 551 |
| D11853_PEA_1_T24 (SEQ ID NO: 71) | 534 | 551 |
| D11853_PEA_1_T25 (SEQ ID NO: 72) | 1383 | 1400 |
| D11853_PEA_1_T26 (SEQ ID NO: 73) | 534 | 551 |

Segment cluster D11853_PEA_{—}1_node_{—}18 (SEQ ID NO:444) according to the present invention is supported by 230 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70), D11853_PEA_{—}1_T25 (SEQ ID NO:72) and D11853_PEA_{—}1_T26 (SEQ ID NO:73). Table 61 below describes the starting and ending position of this segment on each transcript.

TABLE 61
Segment location on transcripts

| | | Segment |
|---|---|---|
| | Segment | ending |
| Transcript name | starting position | position |
| D11853_PEA_1_T1 (SEQ ID NO: 57) | 552 | 582 |
| D11853_PEA_1_T3 (SEQ ID NO: 58) | 552 | 582 |
| D11853_PEA_1_T7 (SEQ ID NO: 59) | 1269 | 1299 |
| D11853_PEA_1_T8 (SEQ ID NO: 60) | 1043 | 1073 |
| D11853_PEA_1_T9 (SEQ ID NO: 61) | 669 | 699 |
| D11853_PEA_1_T10 (SEQ ID NO: 62) | 552 | 582 |
| D11853_PEA_1_T13 (SEQ ID NO: 63) | 552 | 582 |
| D11853_PEA_1_T14 (SEQ ID NO: 64) | 552 | 582 |
| D11853_PEA_1_T15 (SEQ ID NO: 65) | 552 | 582 |
| D11853_PEA_1_T16 (SEQ ID NO: 66) | 701 | 731 |
| D11853_PEA_1_T17 (SEQ ID NO: 67) | 1269 | 1299 |
| D11853_PEA_1_T19 (SEQ ID NO: 68) | 552 | 582 |
| D11853_PEA_1_T21 (SEQ ID NO: 69) | 391 | 421 |
| D11853_PEA_1_T23 (SEQ ID NO: 70) | 701 | 731 |
| D11853_PEA_1_T25 (SEQ ID NO: 72) | 1401 | 1431 |
| D11853_PEA_1_T26 (SEQ ID NO: 73) | 552 | 582 |

Segment cluster D11853_PEA_{—}1_node_{—}19 (SEQ ID NO:445) according to the present invention can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70) and D11853_PEA_{—}1_T25 (SEQ ID NO:72). Table 62 below describes the starting and ending position of this segment on each transcript.

TABLE 62
Segment location on transcripts

| | | Segment |
|---|---|---|
| | Segment | ending |
| Transcript name | starting position | position |
| D11853_PEA_1_T1 (SEQ ID NO: 57) | 583 | 587 |
| D11853_PEA_1_T3 (SEQ ID NO: 58) | 583 | 587 |
| D11853_PEA_1_T7 (SEQ ID NO: 59) | 1300 | 1304 |
| D11853_PEA_1_T8 (SEQ ID NO: 60) | 1074 | 1078 |
| D11853_PEA_1_T9 (SEQ ID NO: 61) | 700 | 704 |
| D11853_PEA_1_T10 (SEQ ID NO: 62) | 583 | 587 |
| D11853_PEA_1_T13 (SEQ ID NO: 63) | 583 | 587 |
| D11853_PEA_1_T14 (SEQ ID NO: 64) | 583 | 587 |
| D11853_PEA_1_T15 (SEQ ID NO: 65) | 583 | 587 |
| D11853_PEA_1_T16 (SEQ ID NO: 66) | 732 | 736 |
| D11853_PEA_1_T17 (SEQ ID NO: 67) | 1300 | 1304 |
| D11853_PEA_1_T19 (SEQ ID NO: 68) | 583 | 587 |
| D11853_PEA_1_T21 (SEQ ID NO: 69) | 422 | 426 |
| D11853_PEA_1_T23 (SEQ ID NO: 70) | 732 | 736 |
| D11853_PEA_1_T25 (SEQ ID NO: 72) | 1432 | 1436 |

Segment cluster D11853_PEA_{—}1_node_{—}20 (SEQ ID NO:446) according to the present invention is supported by 257 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70) and D11853_PEA_{—}1_T25 (SEQ ID NO:72). Table 63 below describes the starting and ending position of this segment on each transcript.

TABLE 63
Segment location on transcripts

| | | Segment |
|---|---|---|
| | Segment | ending |
| Transcript name | starting position | position |
| D11853_PEA_1_T1 (SEQ ID NO: 57) | 588 | 686 |
| D11853_PEA_1_T3 (SEQ ID NO: 58) | 588 | 686 |
| D11853_PEA_1_T7 (SEQ ID NO: 59) | 1305 | 1403 |
| D11853_PEA_1_T8 (SEQ ID NO: 60) | 1079 | 1177 |
| D11853_PEA_1_T9 (SEQ ID NO: 61) | 705 | 803 |
| D11853_PEA_1_T10 (SEQ ID NO: 62) | 588 | 686 |
| D11853_PEA_1_T13 (SEQ ID NO: 63) | 588 | 686 |
| D11853_PEA_1_T14 (SEQ ID NO: 64) | 588 | 686 |
| D11853_PEA_1_T15 (SEQ ID NO: 65) | 588 | 686 |
| D11853_PEA_1_T16 (SEQ ID NO: 66) | 737 | 835 |
| D11853_PEA_1_T17 (SEQ ID NO: 67) | 1305 | 1403 |
| D11853_PEA_1_T19 (SEQ ID NO: 68) | 588 | 686 |
| D11853_PEA_1_T21 (SEQ ID NO: 69) | 427 | 525 |
| D11853_PEA_1_T23 (SEQ ID NO: 70) | 737 | 835 |
| D11853_PEA_1_T25 (SEQ ID NO: 72) | 1437 | 1535 |

Segment cluster D11853_PEA_{—}1_node_{—}24 (SEQ ID NO:447) according to the present invention is supported by 254 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70), D11853_PEA_{—}1_T24 (SEQ ID NO:71) and D11853_PEA_{—}1_T25 (SEQ ID NO:72). Table 64 below describes the starting and ending position of this segment on each transcript.

TABLE 64
Segment location on transcripts

| | | Segment |
|---|---|---|
| | Segment | ending |
| Transcript name | starting position | position |
| D11853_PEA_1_T1 (SEQ ID NO: 57) | 832 | 911 |
| D11853_PEA_1_T3 (SEQ ID NO: 58) | 832 | 911 |
| D11853_PEA_1_T7 (SEQ ID NO: 59) | 1549 | 1628 |
| D11853_PEA_1_T8 (SEQ ID NO: 60) | 1323 | 1402 |
| D11853_PEA_1_T9 (SEQ ID NO: 61) | 949 | 1028 |
| D11853_PEA_1_T10 (SEQ ID NO: 62) | 832 | 911 |
| D11853_PEA_1_T13 (SEQ ID NO: 63) | 955 | 1034 |
| D11853_PEA_1_T14 (SEQ ID NO: 64) | 832 | 911 |
| D11853_PEA_1_T15 (SEQ ID NO: 65) | 955 | 1034 |
| D11853_PEA_1_T16 (SEQ ID NO: 66) | 981 | 1060 |
| D11853_PEA_1_T17 (SEQ ID NO: 67) | 1549 | 1628 |
| D11853_PEA_1_T19 (SEQ ID NO: 68) | 976 | 1055 |
| D11853_PEA_1_T21 (SEQ ID NO: 69) | 671 | 750 |
| D11853_PEA_1_T23 (SEQ ID NO: 70) | 1125 | 1204 |
| D11853_PEA_1_T24 (SEQ ID NO: 71) | 697 | 776 |
| D11853_PEA_1_T25 (SEQ ID NO: 72) | 1681 | 1760 |

Segment cluster D11853_PEA_{—}1_node_{—}28 (SEQ ID NO:448) according to the present invention can be found in the following transcript(s): D11853_PEA_{—}1_T3 (SEQ ID NO:58), D11853_PEA_{—}1_T25 (SEQ ID NO:72) and D11853_PEA_{—}1_T26 (SEQ ID NO:73). Table 65 below describes the starting and ending position of this segment on each transcript.

TABLE 65
Segment location on transcripts

| | | Segment |
|---|---|---|
| | Segment | ending |
| Transcript name | starting position | position |
| D11853_PEA_1_T3 (SEQ ID NO: 58) | 1461 | 1465 |
| D11853_PEA_1_T25 (SEQ ID NO: 72) | 2515 | 2519 |
| D11853_PEA_1_T26 (SEQ ID NO: 73) | 583 | 587 |

Segment cluster D11853_PEA_{—}1_node_{—}29 (SEQ ID NO:449) according to the present invention is supported by 248 libraries. The number of libraries was determined as previously described. This segment can be found in the following transcript(s): D11853_PEA_{—}1_T1 (SEQ ID NO:57), D11853_PEA_{—}1_T3 (SEQ ID NO:58), D11853_PEA_{—}1_T7 (SEQ ID NO:59), D11853_PEA_{—}1_T8 (SEQ ID NO:60), D11853_PEA_{—}1_T9 (SEQ ID NO:61), D11853_PEA_{—}1_T10 (SEQ ID NO:62), D11853_PEA_{—}1_T13 (SEQ ID NO:63), D11853_PEA_{—}1_T14 (SEQ ID NO:64), D11853_PEA_{—}1_T15 (SEQ ID NO:65), D11853_PEA_{—}1_T16 (SEQ ID NO:66), D11853_PEA_{—}1_T17 (SEQ ID NO:67), D11853_PEA_{—}1_T19 (SEQ ID NO:68), D11853_PEA_{—}1_T21 (SEQ ID NO:69), D11853_PEA_{—}1_T23 (SEQ ID NO:70), D11853_PEA_{—}1_T24 (SEQ ID NO:71), D11853_PEA_{—}1_T25 (SEQ ID NO:72) and D11853_PEA_{—}1_T26 (SEQ ID NO:73). Table 66 below describes the starting and ending position of this segment on each transcript.

TABLE 66
Segment location on transcripts

| | | Segment |
|---|---|---|
| | Segment | ending |
| Transcript name | starting position | position |
| D11853_PEA_1_T1 (SEQ ID NO: 57) | 1041 | 1087 |
| D11853_PEA_1_T3 (SEQ ID NO: 58) | 1466 | 1512 |
| D11853_PEA_1_T7 (SEQ ID NO: 59) | 1758 | 1804 |
| D11853_PEA_1_T8 (SEQ ID NO: 60) | 1532 | 1578 |
| D11853_PEA_1_T9 (SEQ ID NO: 61) | 1158 | 1204 |
| D11853_PEA_1_T10 (SEQ ID NO: 62) | 1246 | 1292 |
| D11853_PEA_1_T13 (SEQ ID NO: 63) | 1164 | 1210 |
| D11853_PEA_1_T14 (SEQ ID NO: 64) | 912 | 958 |
| D11853_PEA_1_T15 (SEQ ID NO: 65) | 1369 | 1415 |
| D11853_PEA_1_T16 (SEQ ID NO: 66) | 1190 | 1236 |
| D11853_PEA_1_T17 (SEQ ID NO: 67) | 1758 | 1804 |
| D11853_PEA_1_T19 (SEQ ID NO: 68) | 1185 | 1231 |
| D11853_PEA_1_T21 (SEQ ID NO: 69) | 880 | 926 |
| D11853_PEA_1_T23 (SEQ ID NO: 70) | 1334 | 1380 |
| D11853_PEA_1_T24 (SEQ ID NO: 71) | 906 | 952 |
| D11853_PEA_1_T25 (SEQ ID NO: 72) | 2520 | 2566 |
| D11853_PEA_1_T26 (SEQ ID NO: 73) | 588 | 634 |

### Variant Protein Alignment to the Previously Known Protein:

Sequence name: Q9P042 (SEQ ID NO: 639)
Sequence documentation:
Alignment of:
D11853_PEA_1_P1 (SEQ ID NO: 588) × Q9P042 (SEQ ID NO: 639) . .
Alignment segment 1/1:

| | | | |
|---|---|---|---|
| Quality: | 3115.00 | Escore: | 0 |
| Matching length: | 330 | Total length: | 330 |
| Matching Percent Similarity: | 99.70 | Matching Percent Identity: | 99.70 |
| Total Percent Similarity: | 99.70 | Total Percent Identity: | 99.70 |
| Gaps: | 0 | | |

Alignment:

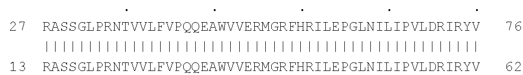

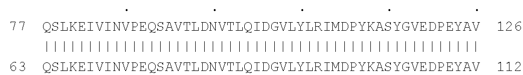

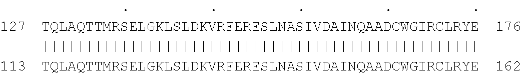

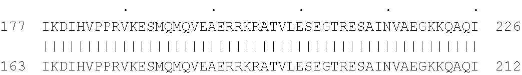

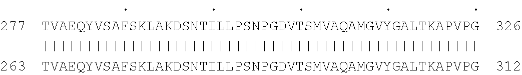

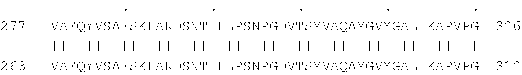

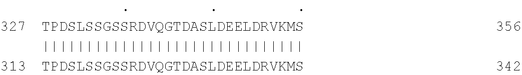

Sequence name: BAC85377 (SEQ ID NO: 640)
Sequence documentation:
Alignment of:
D11853_PEA_1_P1 (SEQ ID NO: 588) × BAC85377 (SEQ ID NO: 640)
Alignment segment 1/1:

| | | | |
|---|---|---|---|
| Quality: | 1512.00 | Escore: | 0 |
| Matching length: | 159 | Total length: | 159 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

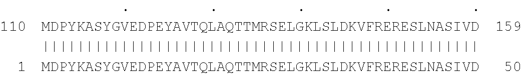

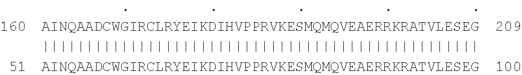

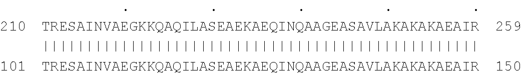

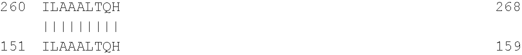

Sequence name: Q96FY2 (SEQ ID NO: 638)
Sequence documentation:
Alignment of:
D11853_PEA_1_P1 (SEQ ID NO: 588) × Q96FY2 (SEQ ID NO: 638) . .
Alignment segment 1/1:

| | | | |
|---|---|---|---|
| Quality: | 3343.00 | Escore: | 0 |
| Matching length: | 356 | Total length: | 356 |
| Matching Percent Similarity: | 99.72 | Matching Percent Identity: | 99.72 |
| Total Percent Similarity: | 99.72 | Total Percent Identity: | 99.72 |
| Gaps: | 0 | | |

Alignment:

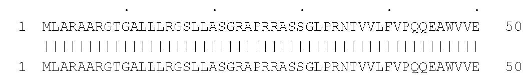

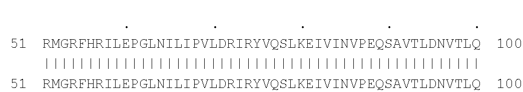

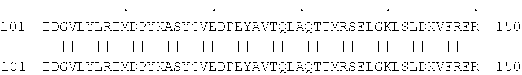

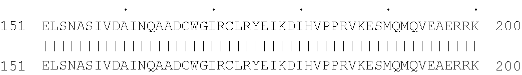

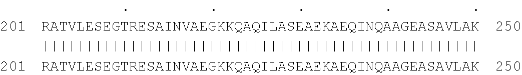

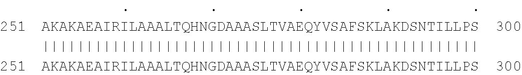

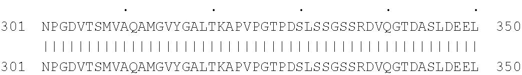

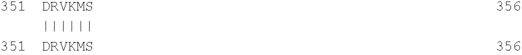

Sequence name: Q9P042 (SEQ ID NO: 639)
Sequence documentation:
Alignment of:
D11853_PEA_1_P2 (SEQ ID NO: 589) × Q9P042 (SEQ ID NO: 639) . .
Alignment segment 1/1:

| | | | |
|---|---|---|---|
| Quality: | 2691.00 | Escore: | 0 |
| Matching length: | 285 | Total length: | 285 |
| Matching Percent Similarity: | 99.65 | Matching Percent Identity: | 99.65 |
| Total Percent Similarity: | 99.65 | Total Percent Identity: | 99.65 |
| Gaps: | 0 | | |

Alignment:

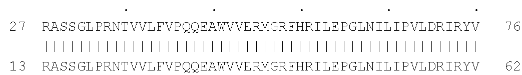

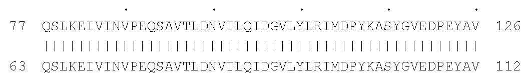

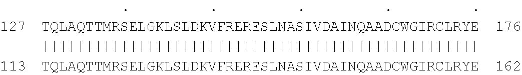

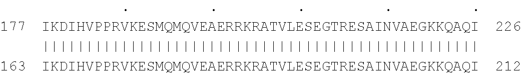

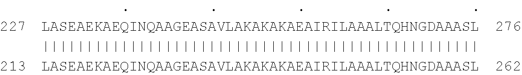

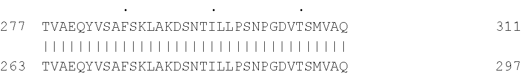

Sequence name: BAC85377 (SEQ ID NO: 640)
Sequence documentation:
Alignment of:
D11853_PEA_1_P2 (SEQ ID NO: 589) × BAC85377 (SEQ ID NO: 640)
Alignment segment 1/1:

| | | | |
|---|---|---|---|
| Quality: | 1512.00 | Escore: | 0 |
| Matching length: | 159 | Total length: | 159 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

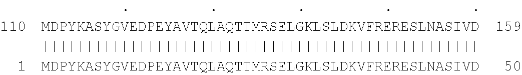

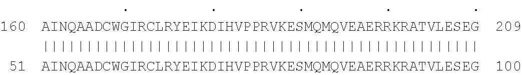

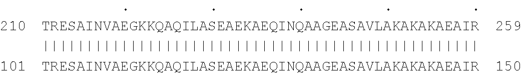

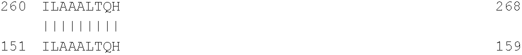

Sequence name: Q96FY2 (SEQ ID NO: 638)
Sequence documentation:
Alignment of:
D11853_PEA_1_P2 (SEQ ID NO: 589) × Q96FY2 (SEQ ID NO: 638) . .
Alignment segment 1/1:

| | | | |
|---|---|---|---|
| Quality: | 2919.00 | Escore: | 0 |
| Matching length: | 311 | Total length: | 311 |
| Matching Percent Similarity: | 99.68 | Matching Percent Identity: | 99.68 |
| Total Percent Similarity: | 99.68 | Total Percent Identity: | 99.68 |
| Gaps: | 0 | | |

Alignment:

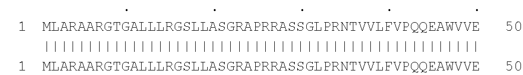

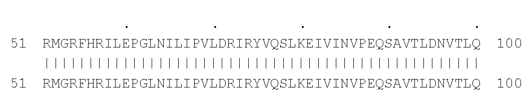

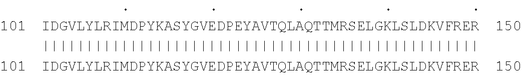

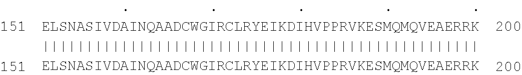

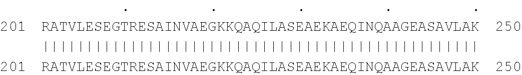

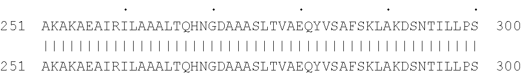

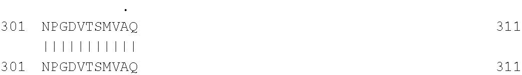

Sequence name: Q9UJZ1 (SEQ ID NO: 637)
Sequence documentation:
Alignment of:
D11853_PEA_1_P2 (SEQ ID NO: 589) × Q9UJZ1 (SEQ ID NO: 637) . .
Alignment segment 1/1:

| | | | |
|---|---|---|---|
| Quality: | 2934.00 | Escore: | 0 |
| Matching length: | 311 | Total length: | 311 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

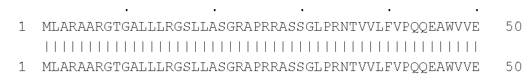

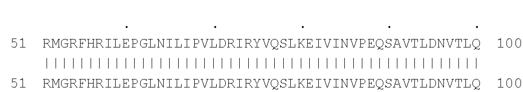

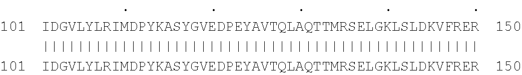

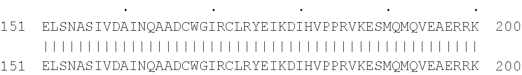

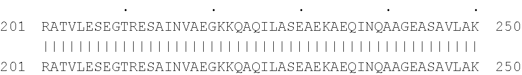

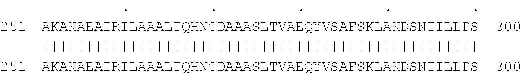

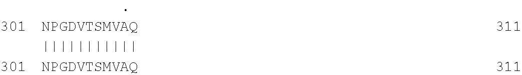

Sequence name: Q9P042 (SEQ ID NO: 639)
Sequence documentation:
Alignment of:
D11853_PEA_1_P7 (SEQ ID NO: 590) × Q9P042 (SEQ ID NO: 639) . .
Alignment segment 1/1:

| | | | |
|---|---|---|---|
| Quality: | 2290.00 | Escore: | 0 |
| Matching length: | 242 | Total length: | 242 |
| Matching Percent Similarity: | 99.59 | Matching Percent Identity: | 99.59 |
| Total Percent Similarity: | 99.59 | Total Percent Identity: | 99.59 |
| Gaps: | 0 | | |

Alignment:

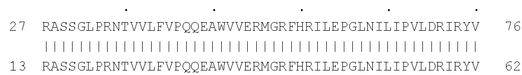

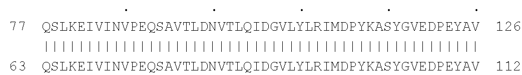

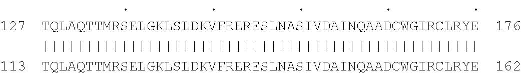

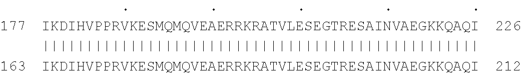

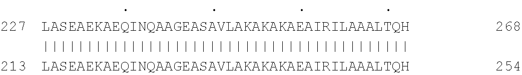

Sequence name: BAC85377 (SEQ ID NO: 640)
Sequence documentation:
Alignment of:
D11853_PEA_1_P7 (SEQ ID NO: 590) × BAC85377 (SEQ ID NO: 640)
Alignment segment 1/1:

| | | | |
|---|---|---|---|
| Quality: | 1724.00 | Escore: | 0 |
| Matching length: | 181 | Total length: | 181 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

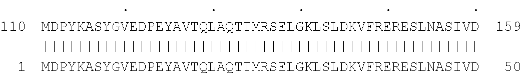

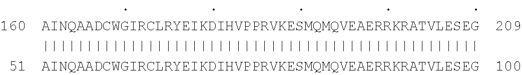

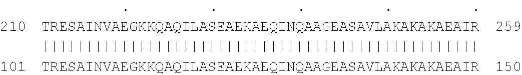

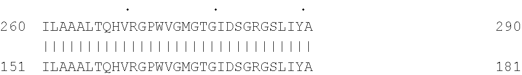

Sequence name: Q96FY2 (SEQ ID NO: 638)
Sequence documentation:
Alignment of:
D11853_PEA_1_P7 (SEQ ID NO: 590) × Q96FY2 (SEQ ID NO: 638) . .
Alignment segment 1/1:

| | | | |
|---|---|---|---|
| Quality: | 2518.00 | Escore: | 0 |
| Matching length: | 268 | Total length: | 268 |
| Matching Percent Similarity: | 99.63 | Matching Percent Identity: | 99.63 |
| Total Percent Similarity: | 99.63 | Total Percent Identity: | 99.63 |
| Gaps: | 0 | | |

Alignment:

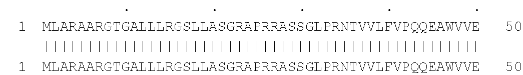

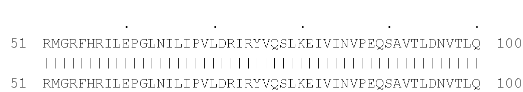

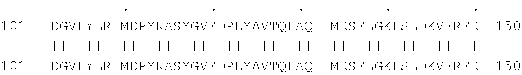

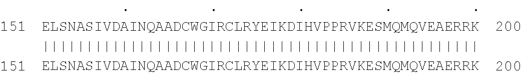

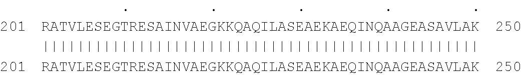

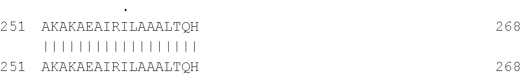

Sequence name: Q9UJZ1 (SEQ ID NO: 637)
Sequence documentation:
Alignment of:
D11853_PEA_1_P7 (SEQ ID NO: 590) × Q9UJZ1 (SEQ ID NO: 637) . .
Alignment segment 1/1:

| | | | |
|---|---|---|---|
| Quality: | 2533.00 | Escore: | 0 |
| Matching length: | 268 | Total length: | 268 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

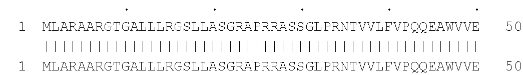

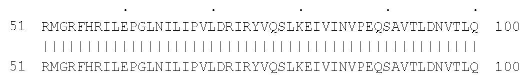

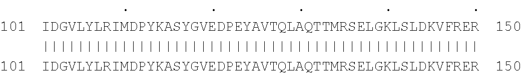

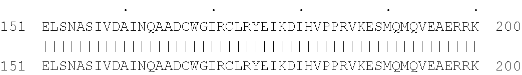

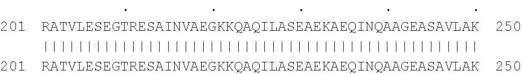

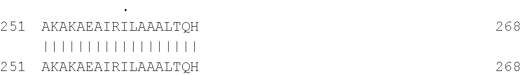

Sequence name: Q9P042 (SEQ ID NO: 639)
Sequence documentation:
Alignment of:
D11853_PEA_1_P9 (SEQ ID NO: 591) × Q9P042 (SEQ ID NO: 639) . .
Alignment segment 1/1:

| | | | |
|---|---|---|---|
| Quality: | 3015.00 | Escore: | 0 |
| Matching length: | 330 | Total length: | 371 |
| Matching Percent Similarity: | 99.70 | Matching Percent Identity: | 99.70 |
| Total Percent Similarity: | 88.68 | Total Percent Identity: | 88.68 |
| Gaps: | 1 | | |

Alignment:

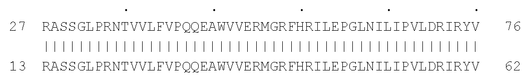

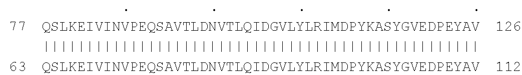

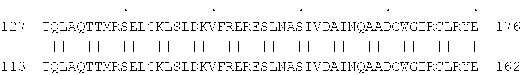

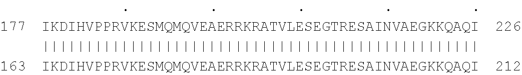

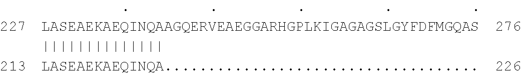

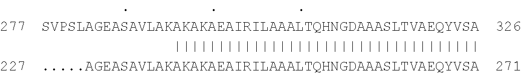

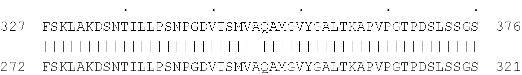

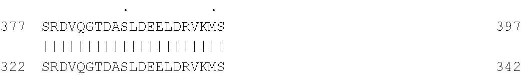

Sequence name: BAC85377 (SEQ ID NO: 640)
Sequence documentation:
Alignment of:
D11853_PEA_1_P9 (SEQ ID NO: 591) × BAC85377 (SEQ ID NO: 640)
Alignment segment 1/1:

| | | | |
|---|---|---|---|
| Quality: | 1412.00 | Escore: | 0 |
| Matching length: | 159 | Total length: | 200 |
| Matching Percent Similarity: | 100.00 | Matching Percent Identity: | 100.00 |
| Total Percent Similarity: | 79.50 | Total Percent Identity: | 79.50 |
| Gaps: | 1 | | |

Alignment:

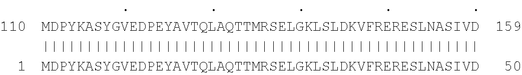

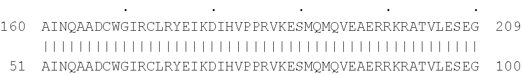

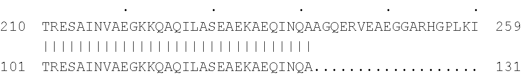

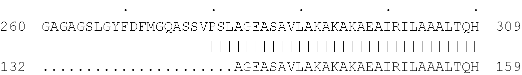

Sequence name: Q96FY2 (SEQ ID NO: 638)
Sequence documentation:
Alignment of:
D11853_PEA_1_P9 (SEQ ID NO: 591) × Q96FY2 (SEQ ID NO: 638) . .
Alignment segment 1/1:

| | | | |
|---|---|---|---|
| Quality: | 3243.00 | Escore: | 0 |
| Matching length: | 356 | Total length: | 397 |
| Matching Percent Similarity: | 99.72 | Matching Percent Identity: | 99.72 |
| Total Percent Similarity: | 89.42 | Total Percent Identity: | 89.42 |
| Gaps: | 1 | | |

Alignment:

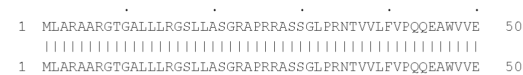

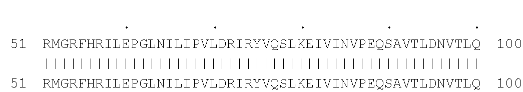

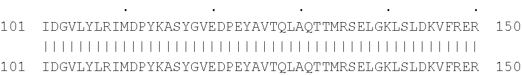

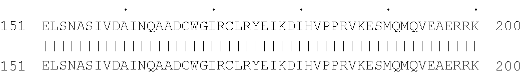

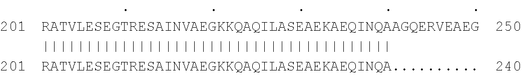

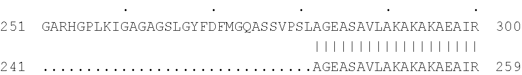

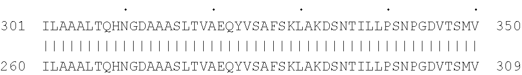

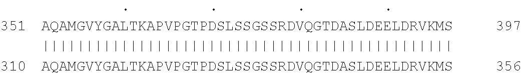

Sequence name: Q9UJZ1 (SEQ ID NO: 637)
Sequence documentation:
Alignment of:
D11853_PEA_1_P9 (SEQ ID NO: 591) × Q9UJZ1 (SEQ ID NO: 637) . .
Alignment segment 1/1:

| | | | |
|---|---|---|---|
| Quality: | 3258.00 | Escore: | 0 |
| Matching length: | 356 | Total length: | 397 |
| Matching Percent Similarity: | 100.00 | Matching Percent Identity: | 100.00 |
| Total Percent Similarity: | 89.67 | Total Percent Identity: | 89.67 |
| Gaps: | 1 | | |

Alignment:

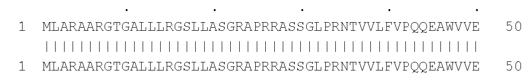

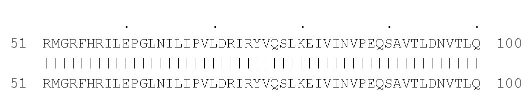

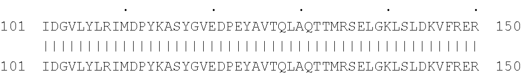

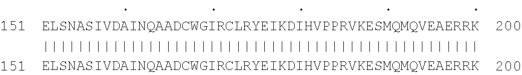

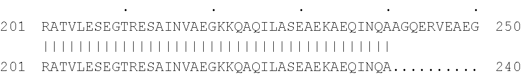

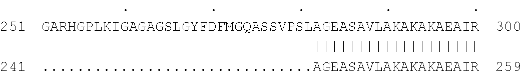

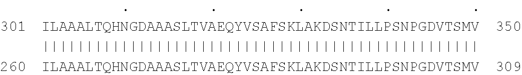

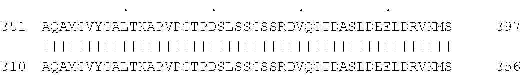

Sequence name: Q9P042 (SEQ ID NO: 639)
Sequence documentation:
Alignment of:
D11853_PEA_1_P10 (SEQ ID NO: 592) × Q9P042 (SEQ ID NO: 639)
Alignment segment 1/1:

| | | | |
|---|---|---|---|
| Quality: | 2614.00 | Escore: | 0 |
| Matching length: | 287 | Total length: | 330 |
| Matching Percent Similarity: | 99.65 | Matching Percent Identity: | 99.65 |
| Total Percent Similarity: | 86.67 | Total Percent Identity: | 86.67 |
| Gaps: | 1 | | |

Alignment:

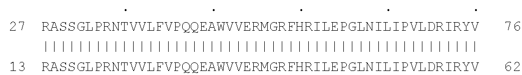

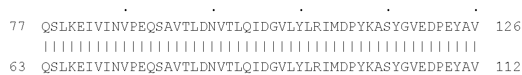

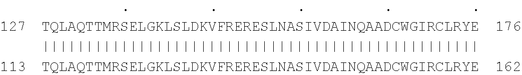

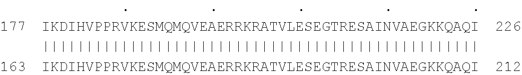

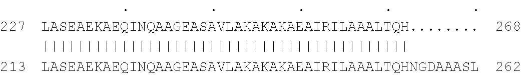

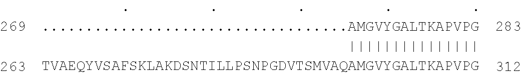

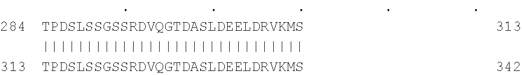

Sequence name: BAC85377 (SEQ ID NO: 640)
Sequence documentation:
Alignment of:
D11853_PEA_1_P10 (SEQ ID NO: 592) × BAC85377 (SEQ ID NO: 640)
Alignment segment 1/1:

| | | | |
|---|---|---|---|
| Quality: | 1515.00 | Escore: | 0 |
| Matching length: | 162 | Total length: | 162 |
| Matching Percent Similarity: | 98.77 | Matching Percent Identity: | 98.77 |
| Total Percent Similarity: | 98.77 | Total Percent Identity: | 98.77 |
| Gaps: | 0 | | |

Alignment:

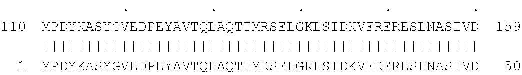

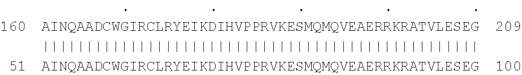

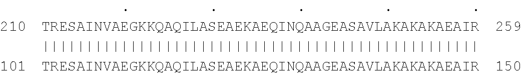

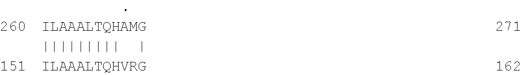

Sequence name: Q96FY2 (SEQ ID NO: 638)
Sequence documentation:
Alignment of:
D11853_PEA_1_P10 (SEQ ID NO: 592) × Q96FY2 (SEQ ID NO: 638)
Alignment segment 1/1:

| | | | |
|---|---|---|---|
| Quality: | 2842.00 | Escore: | 0 |
| Matching length: | 313 | Total length: | 356 |
| Matching Percent Similarity: | 99.68 | Matching Percent Identity: | 99.68 |
| Total Percent Similarity: | 87.64 | Total Percent Identity: | 87.64 |
| Gaps: | 1 | | |

Alignment:

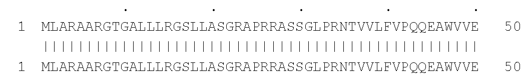

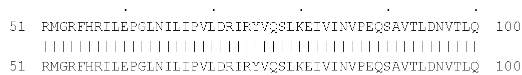

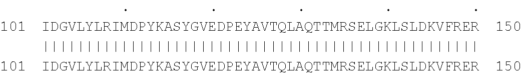

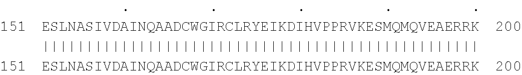

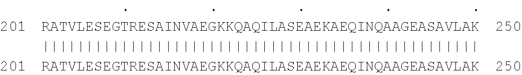

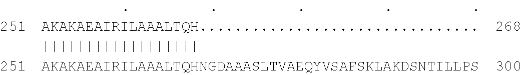

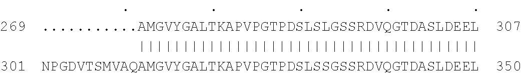

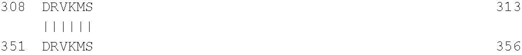

Sequence name: Q9UJZ1 (SEQ ID NO: 637)
Sequence documentation:
Alignment of:
D11853_PEA_1_P10 (SEQ ID NO: 592) × Q9UJZ1 (SEQ ID NO: 637)
Alignment segment 1/1:

| | | | |
|---|---|---|---|
| Quality: | 2857.00 | Escore: | 0 |
| Matching length: | 313 | Total length: | 356 |
| Matching Percent Similarity: | 100.00 | Matching Percent Identity: | 100.00 |
| Total Percent Similarity: | 87.92 | Total Percent Identity: | 87.92 |
| Gaps: | 1 | | |

Alignment:

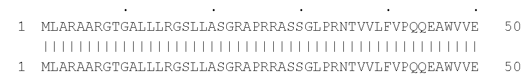

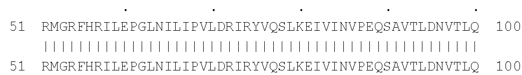

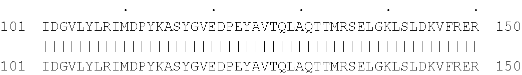

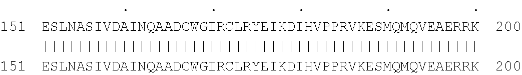

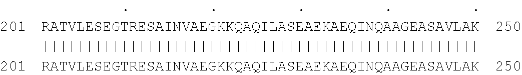

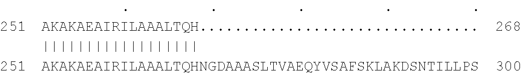

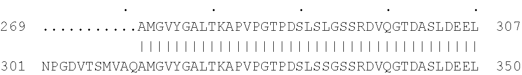

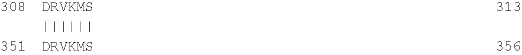

Sequence name: Q9P042 (SEQ ID NO: 639)
Sequence documentation:
Alignment of:
D11853_PEA_1_P11 (SEQ ID NO: 593) × Q9P042 (SEQ ID NO: 639)
Alignment segment 1/1:

| | | | |
|---|---|---|---|
| Quality: | 2190.00 | Escore: | 0 |
| Matching length: | 242 | Total length: | 283 |
| Matching Percent Similarity: | 99.59 | Matching Percent Identity: | 99.59 |
| Total Percent Similarity: | 85.16 | Total Percent Identity: | 85.16 |
| Gaps: | 1 | | |

Alignment:

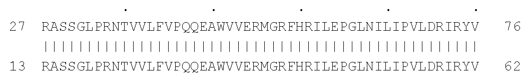

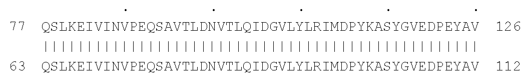

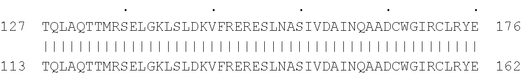

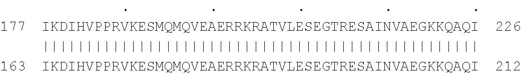

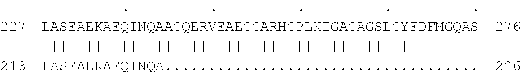

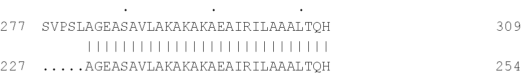

Sequence name: BAC85377 (SEQ ID NO: 640)
Sequence documentation:
Alignment of:
D11853_PEA_1_P11 (SEQ ID NO: 593) × BAC85377 (SEQ ID NO: 640)
Alignment segment 1/1:

| | | | |
|---|---|---|---|
| Quality: | 1624.00 | Escore: | 0 |
| Matching length: | 181 | Total length: | 222 |
| Matching Percent Similarity: | 100.00 | Matching Percent Identity: | 100.00 |
| Total Percent Similarity: | 81.53 | Total Percent Identity: | 81.53 |
| Gaps: | 1 | | |

Alignment:

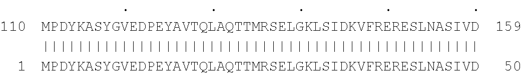

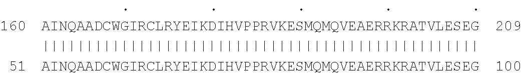

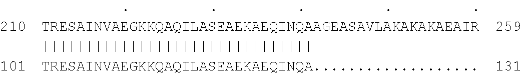

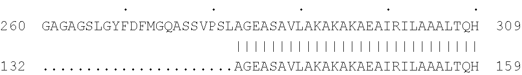

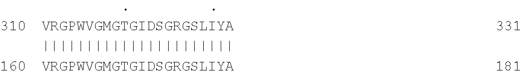

Sequence name: Q96FY2 (SEQ ID NO: 638)
Sequence documentation:
Alignment of:
D11853_PEA_1_P11 (SEQ ID NO: 593) × Q96FY2 (SEQ ID NO: 638)
Alignment segment 1/1:

| | | | |
|---|---|---|---|
| Quality: | 2418.00 | Escore: | 0 |
| Matching length: | 268 | Total length: | 309 |
| Matching Percent Similarity: | 99.63 | Matching Percent Identity: | 99.63 |
| Total Percent Similarity: | 86.41 | Total Percent Identity: | 86.41 |
| Gaps: | 1 | | |

Alignment:

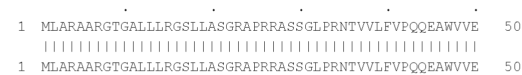

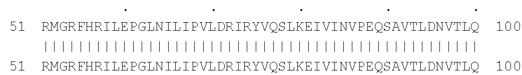

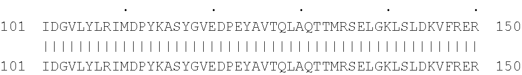

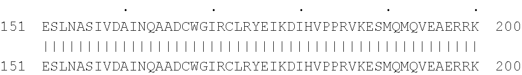

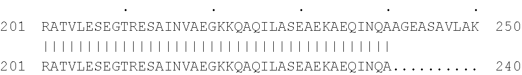

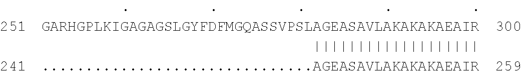

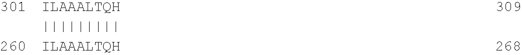

Sequence name: Q9UJZ1 (SEQ ID NO: 637)
Sequence documentation:
Alignment of:
D11853_PEA_1_P11 (SEQ ID NO: 593) × Q9UJZ1 (SEQ ID NO: 637)
Alignment segment 1/1:

| | | | |
|---|---|---|---|
| Quality: | 2433.00 | Escore: | 0 |
| Matching length: | 268 | Total length: | 309 |
| Matching Percent Similarity: | 100.00 | Matching Percent Identity: | 100.00 |
| Total Percent Similarity: | 86.73 | Total Percent Identity: | 86.73 |
| Gaps: | 1 | | |

Alignment:

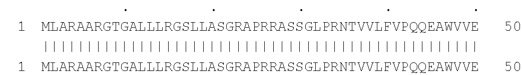

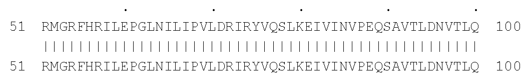

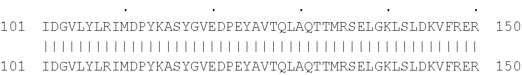

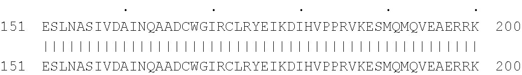

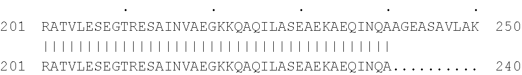

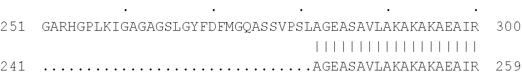

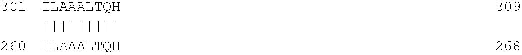

Sequence name: Q9P042 (SEQ ID NO: 639)
Sequence documentation:
Alignment of:
D11853_PEA_1_P12 (SEQ ID NO: 594) × Q9P042 (SEQ ID NO: 639)
Alignment segment 1/1:

| | | | |
|---|---|---|---|
| Quality: | 1167.00 | Escore: | 0 |
| Matching length: | 122 | Total length: | 122 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

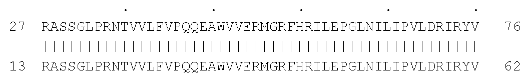

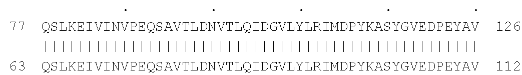

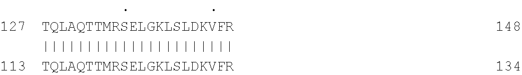

Sequence name: Q96FY2 (SEQ ID NO: 638)
Sequence documentation:
Alignment of:
D11853_PEA_1_P12 (SEQ ID NO: 594) × Q96FY2 (SEQ ID NO: 638)
Alignment segment 1/1:

| | | | |
|---|---|---|---|
| Quality: | 1385.00 | Escore: | 0 |
| Matching length: | 148 | Total length: | 148 |
| Matching Percent Similarity: | 99.32 | Matching Percent Identity: | 99.32 |
| Total Percent Similarity: | 99.32 | Total Percent Identity: | 99.32 |
| Gaps: | 0 | | |

Alignment:

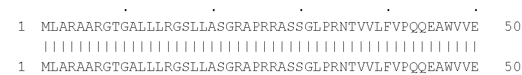

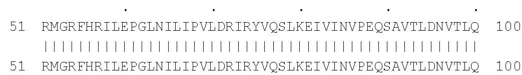

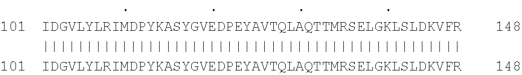

Sequence name: Q9UJZ1 (SEQ ID NO: 637)
Sequence documentation:
Alignment of:
D11853_PEA_1_P12 (SEQ ID NO: 594) × Q9UJZ1 (SEQ ID NO: 637)
Alignment segment 1/1:

| | | | |
|---|---|---|---|
| Quality: | 1400.00 | Escore: | 0 |
| Matching length: | 148 | Total length: | 148 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

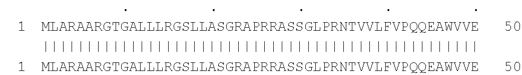

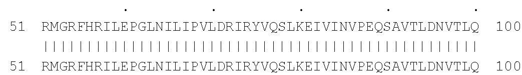

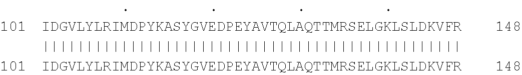

Sequence name: Q9P042 (SEQ ID NO: 639)
Sequence documentation:
Alignment of:
D11853_PEA_1_P14 (SEQ ID NO: 595) × Q9P042 (SEQ ID NO: 639)
Alignment segment 1/1:

| | | | |
|---|---|---|---|
| Quality: | 1628.00 | Escore: | 0 |
| Matching length: | 170 | Total length: | 170 |
| Matching Percent Similarity: | 99.41 | Matching Percent Identity: | 99.41 |
| Total Percent Similarity: | 99.41 | Total Percent Identity: | 99.41 |
| Gaps: | 0 | | |

Alignment:

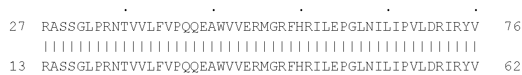

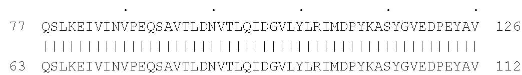

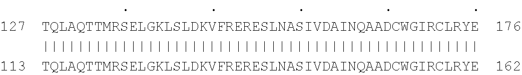

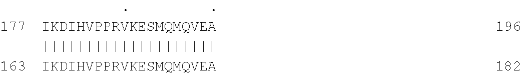

Sequence name: Q96FY2 (SEQ ID NO: 638)
Sequence documentation:
Alignment of:
D11853_PEA_1_P14 (SEQ ID NO: 595) × Q96FY2 (SEQ ID NO: 638)
Alignment segment 1/1:

| | | | |
|---|---|---|---|
| Quality: | 1846.00 | Escore: | 0 |
| Matching length: | 196 | Total length: | 196 |
| Matching Percent Similarity: | 98.98 | Matching Percent Identity: | 98.98 |
| Total Percent Similarity: | 98.98 | Total Percent Identity: | 98.98 |
| Gaps: | 0 | | |

Alignment:

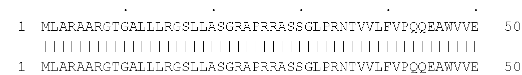

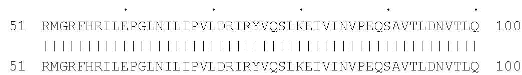

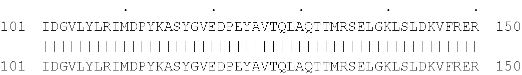

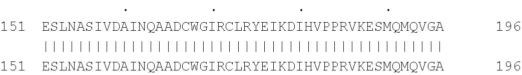

Sequence name: Q9UJZ1 (SEQ ID NO: 637)
Sequence documentation:
Alignment of:
D11853_PEA_1_P14 (SEQ ID NO: 595) × Q9UJZ1 (SEQ ID NO: 637)
Alignment segment 1/1:

| | | | |
|---|---|---|---|
| Quality: | 1861.00 | Escore: | 0 |
| Matching length: | 196 | Total length: | 196 |
| Matching Percent Similarity: | 99.49 | Matching Percent Identity: | 99.49 |
| Total Percent Similarity: | 99.49 | Total Percent Identity: | 99.49 |
| Gaps: | 0 | | |

Alignment:

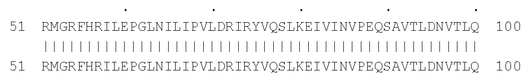

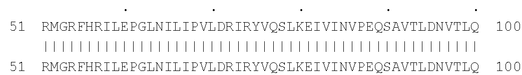

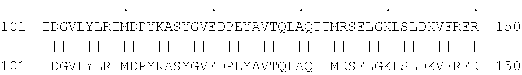

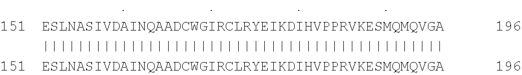

Sequence name: Q9P042 (SEQ ID NO: 639)
Sequence documentation:
Alignment of:
D11853_PEA_1_P16 (SEQ ID NO: 596) × Q9P042 (SEQ ID NO: 639)
Alignment segment 1/1:

| | | | |
|---|---|---|---|
| Quality: | 2564.00 | Escore: | 0 |
| Matching length: | 285 | Total length: | 330 |
| Matching Percent Similarity: | 99.65 | Matching Percent Identity: | 99.65 |
| Total Percent Similarity: | 86.06 | Total Percent Identity: | 86.06 |
| Gaps: | 1 | | |

Alignment:

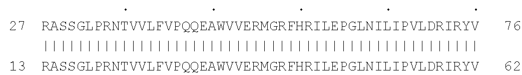

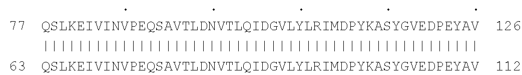

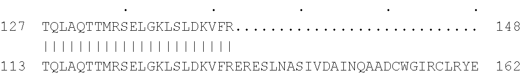

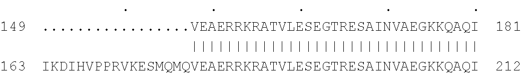

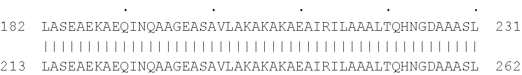

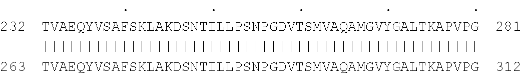

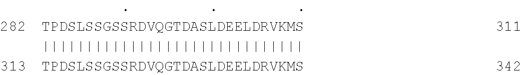

Sequence name: BAC85377 (SEQ ID NO: 640)
Sequence documentation:
Alignment of:
D11853_PEA_1_P16 (SEQ ID NO: 596) × BAC85377 (SEQ ID NO: 640)
Alignment segment 1/1:

| | | | |
|---|---|---|---|
| Quality: | 961.00 | Escore: | 0 |
| Matching length: | 114 | Total length: | 159 |
| Matching Percent Similarity: | 100.00 | Matching Percent Identity: | 100.00 |
| Total Percent Similarity: | 71.70 | Total Percent Identity: | 71.70 |
| Gaps: | 1 | | |

Alignment:

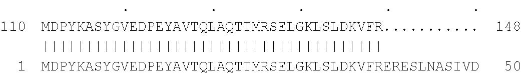

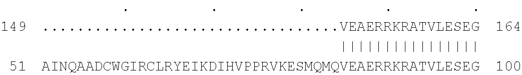

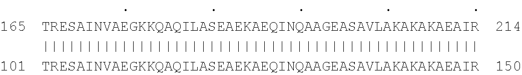

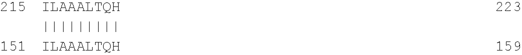

Sequence name: Q96FY2 (SEQ ID NO: 638)
Sequence documentation:
Alignment of:
D11853_PEA_1_P16 (SEQ ID NO: 596) × Q96FY2 (SEQ ID NO: 638)
Alignment segment 1/1:

| | | | |
|---|---|---|---|
| Quality: | 2792.00 | Escore: | 0 |
| Matching length: | 311 | Total length: | 356 |
| Matching Percent Similarity: | 99.68 | Matching Percent Identity: | 99.68 |
| Total Percent Similarity: | 87.08 | Total Percent Identity: | 87.08 |
| Gaps: | 1 | | |

Alignment:

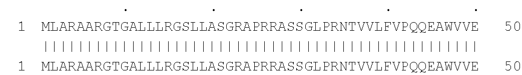

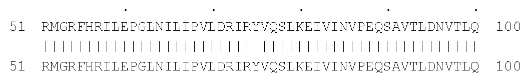

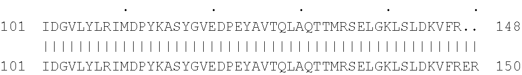

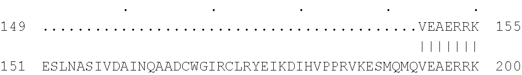

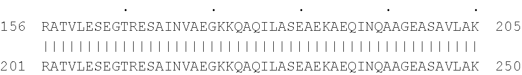

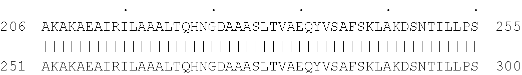

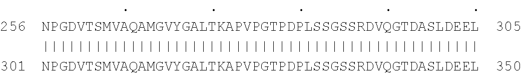

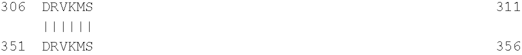

Sequence name: Q9UJZ1 (SEQ ID NO: 637)
Sequence documentation:
Alignment of:
D11853_PEA_1_P16 (SEQ ID NO: 596) × Q9UJZ1 (SEQ ID NO: 637)
Alignment segment 1/1:

| | | | |
|---|---|---|---|
| Quality: | 2807.00 | Escore: | 0 |
| Matching length: | 311 | Total length: | 356 |
| Matching Percent Similarity: | 100.00 | Matching Percent Identity: | 100.00 |
| Total Percent Similarity: | 87.36 | Total Percent Identity: | 87.36 |
| Gaps: | 1 | | |

Alignment:

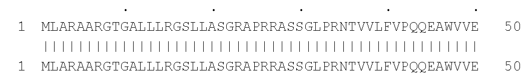

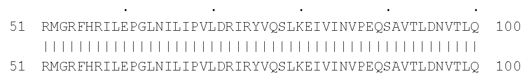

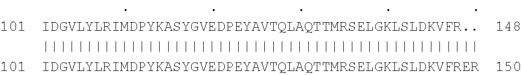

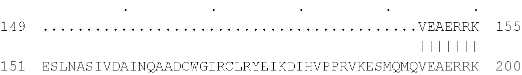

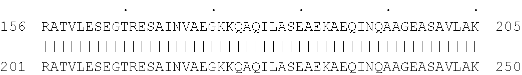

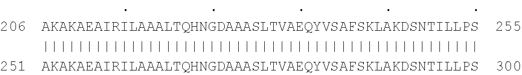

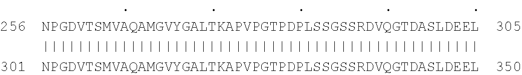

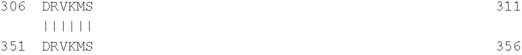

Sequence name: Q9P042 (SEQ ID NO: 639)
Sequence documentation:
Alignment of:
D11853_PEA_1_P18 (SEQ ID NO: 597) × Q9P042 (SEQ ID NO: 639)
Alignment segment 1/1:

| | | | |
|---|---|---|---|
| Quality: | 1601.00 | Escore: | 0 |
| Matching length: | 179 | Total length: | 330 |
| Matching Percent Similarity: | 100.00 | Matching Percent Identity: | 100.00 |
| Total Percent Similarity: | 54.24 | Total Percent Identity: | 54.24 |
| Gaps: | 1 | | |

Alignment:

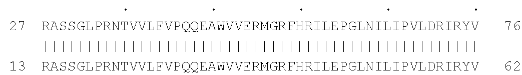

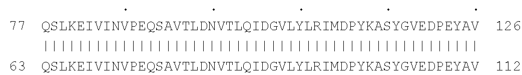

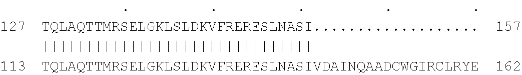

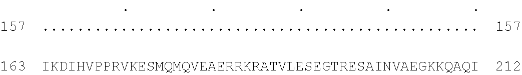

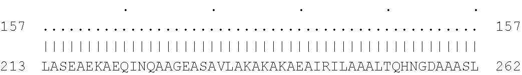

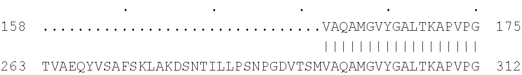

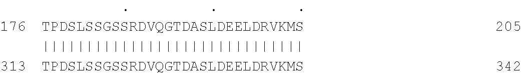

Sequence name: Q96FY2 (SEQ ID NO: 638)
Sequence documentation:
Alignment of:
D11853_PEA_1_P18 (SEQ ID NO: 597) × Q96FY2 (SEQ ID NO: 638)
Alignment segment 1/1:

| | | | |
|---|---|---|---|
| Quality: | 1819.00 | Escore: | 0 |
| Matching length: | 205 | Total length: | 356 |
| Matching Percent Similarity: | 99.51 | Matching Percent Identity: | 99.51 |
| Total Percent Similarity: | 57.30 | Total Percent Identity: | 57.30 |
| Gaps: | 1 | | |

Alignment:

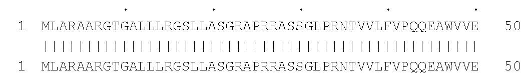

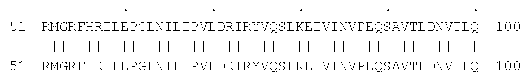

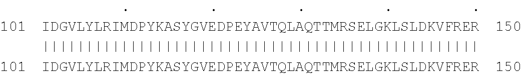

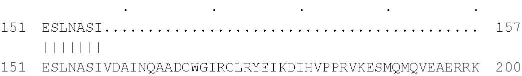

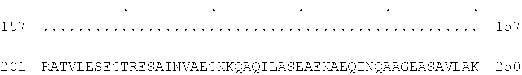

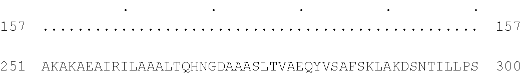

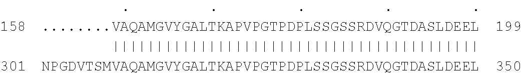

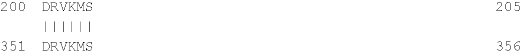

Sequence name: Q9UJZ1 (SEQ ID NO: 637)
Sequence documentation:
Alignment of:
D11853_PEA_1_P18 (SEQ ID NO: 597) × Q9UJZ1 (SEQ ID NO: 637)
Alignment segment 1/1:

| | | | |
|---|---|---|---|
| Quality: | 1834.00 | Escore: | 0 |
| Matching length: | 205 | Total length: | 356 |
| Matching Percent Similarity: | 100.00 | Matching Percent Identity: | 100.00 |
| Total Percent Similarity: | 57.58 | Total Percent Identity: | 57.58 |
| Gaps: | 1 | | |

Alignment:

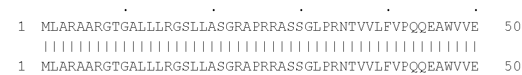

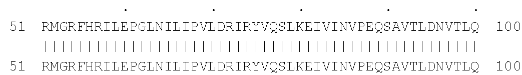

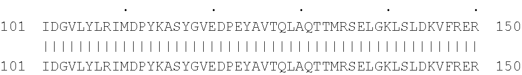

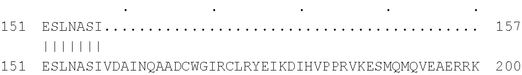

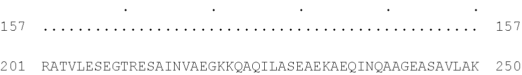

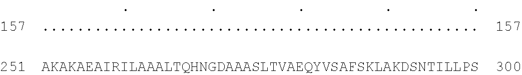

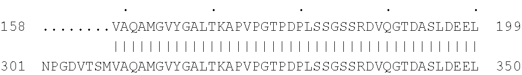

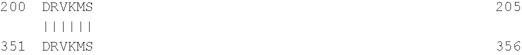

Sequence name: Q9P042 (SEQ ID NO: 639)
Sequence documentation:
Alignment of:
D11853_PEA_1_P19 (SEQ ID NO: 598) × Q9P042 (SEQ ID NO: 639)
Alignment segment 1/1:

| | | | |
|---|---|---|---|
| Quality: | 1110.00 | Escore: | 0 |
| Matching length: | 116 | Total length: | 116 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

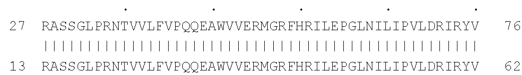

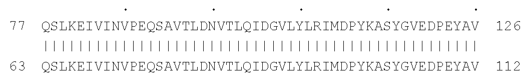

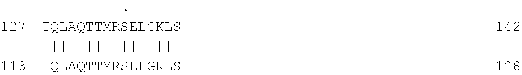

Sequence name: Q96FY2 (SEQ ID NO: 638)
Sequence documentation:
Alignment of:
D11853_PEA_1_P19 (SEQ ID NO: 598) × Q96FY2 (SEQ ID NO: 638)
Alignment segment 1/1:

| | | | |
|---|---|---|---|
| Quality: | 1328.00 | Escore: | 0 |
| Matching length: | 142 | Total length: | 142 |
| Matching Percent Similarity: | 99.30 | Matching Percent Identity: | 99.30 |
| Total Percent Similarity: | 99.30 | Total Percent Identity: | 99.30 |
| Gaps: | 0 | | |

Alignment:

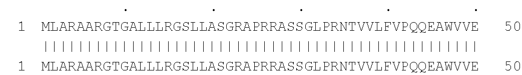

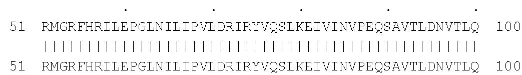

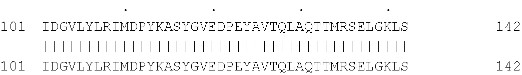

Sequence name: Q9UJZ1 (SEQ ID NO: 637)
Sequence documentation:
Alignment of:
D11853_PEA_1_P19 (SEQ ID NO: 598) × Q9UJZ1 (SEQ ID NO: 637)
Alignment segment 1/1:

| | | | |
|---|---|---|---|
| Quality: | 1343.00 | Escore: | 0 |
| Matching length: | 142 | Total length: | 142 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

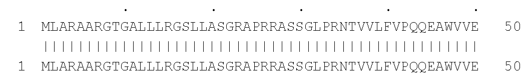

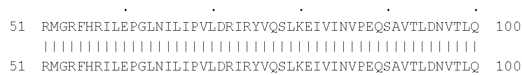

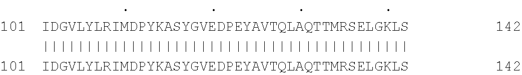

Sequence name: Q96FY2 (SEQ ID NO: 638)
Sequence documentation:
Alignment of:
D11853_PEA_1 P21 (SEQ ID NO: 600) × Q96FY2 (SEQ ID NO: 638)
Alignment segment 1/1:

| | | | |
|---|---|---|---|
| Quality: | 587.00 | Escore: | 0 |
| Matching length: | 68 | Total length: | 68 |
| Matching Percent Similarity: | 95.59 | Matching Percent Identity: | 92.65 |
| Total Percent Similarity: | 95.59 | Total Percent Identity: | 92.65 |
| Gaps: | 0 | | |

Alignment:

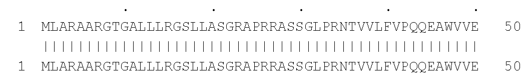

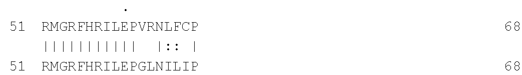

Sequence name: Q9UJZ1 (SEQ ID NO: 637)
Sequence documentation:
Alignment of:
D11853_PEA_1_P21 (SEQ ID NO: 600) × Q9UJZ1 (SEQ ID NO: 637)
Alignment segment 1/1:

| | | | |
|---|---|---|---|
| Quality: | 587.00 | Escore: | 0 |
| Matching length: | 68 | Total length: | 68 |
| Matching Percent Similarity: | 95.59 | Matching Percent Identity: | 92.65 |
| Total Percent Similarity: | 95.59 | Total Percent Identity: | 92.65 |
| Gaps: | 0 | | |

Alignment:

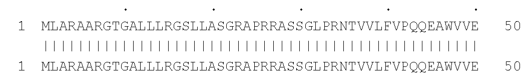

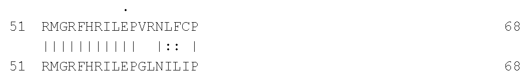

Sequence name: Q9P042 (SEQ ID NO: 639)
Sequence documentation:
Alignment of:
D11853_PEA_1_P22 (SEQ ID NO: 601) × Q9P042 (SEQ ID NO: 639)
Alignment segment 1/1:

| | | | |
|---|---|---|---|
| Quality: | 348.00 | Escore: | 0 |
| Matching length: | 37 | Total length: | 37 |
| Matching Percent Similarity: | 97.30 | Matching Percent Identity: | 97.30 |
| Total Percent Similarity: | 97.30 | Total Percent Identity: | 97.30 |
| Gaps: | 0 | | |

Alignment:

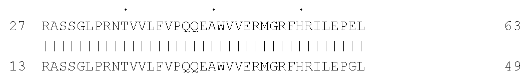

Sequence name: Q96FY2 (SEQ ID NO: 638)
Sequence documentation:
Alignment of:
D11853_PEA_1_P22 (SEQ ID NO: 601) × Q96FY2 (SEQ ID NO: 638)
Alignment segment 1/1:

| | | | |
|---|---|---|---|
| Quality: | 581.00 | Escore: | 0 |
| Matching length: | 63 | Total length: | 63 |
| Matching Percent Similarity: | 98.41 | Matching Percent Identity: | 98.41 |
| Total Percent Similarity: | 98.41 | Total Percent Identity: | 98.41 |
| Gaps: | 0 | | |

Alignment:

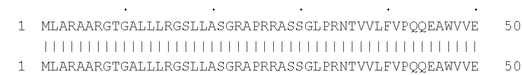

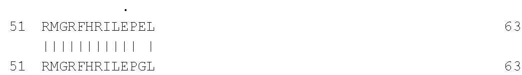

Sequence name: Q9UJZ1 (SEQ ID NO: 637)
Sequence documentation:
Alignment of:
D11853_PEA_1_P22 (SEQ ID NO: 601) × Q9UJZ1 (SEQ ID NO: 637)
Alignment segment 1/1:

| | | | |
|---|---|---|---|
| Quality: | 581.00 | Escore: | 0 |
| Matching length: | 63 | Total length: | 63 |
| Matching Percent Similarity: | 98.41 | Matching Percent Identity: | 98.41 |
| Total Percent Similarity: | 98.41 | Total Percent Identity: | 98.41 |
| Gaps: | 0 | | |

Alignment:

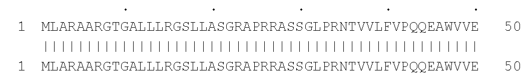

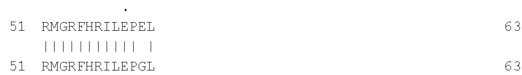

Sequence name: Q9P042 (SEQ ID NO: 639)
Sequence documentation:
Alignment of:
D11853_PEA_1_P24 (SEQ ID NO: 602) × Q9P042 (SEQ ID NO: 639)
Alignment segment 1/1:

| | | | |
|---|---|---|---|
| Quality: | 650.00 | Escore: | 0 |
| Matching length: | 68 | Total length: | 68 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

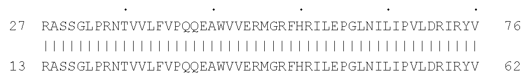

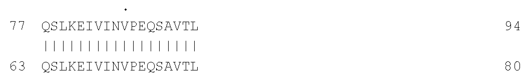

Sequence name: Q96FY2 (SEQ ID NO: 638)
Sequence documentation:
Alignment of:
D11853_PEA_1_P24 (SEQ ID NO: 602) × Q96FY2 (SEQ ID NO: 638)
Alignment segment 1/1:

| | | | |
|---|---|---|---|
| Quality: | 883.00 | Escore: | 0 |
| Matching length: | 94 | Total length: | 94 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

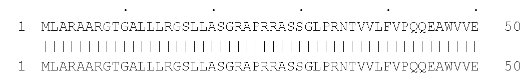

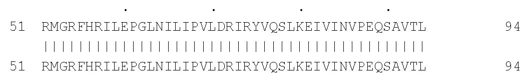

Sequence name: Q9UJZ1 (SEQ ID NO: 637)
Sequence documentation:
Alignment of:
D11853_PEA_1_P24 (SEQ ID NO: 602) × Q9UJZ1 (SEQ ID NO: 637)
Alignment segment 1/1:

| | | | |
|---|---|---|---|
| Quality: | 883.00 | Escore: | 0 |
| Matching length: | 94 | Total length: | 94 |
| Matching Percent Similarity: | 100.00 | Matching Percent Identity: | 100.00 |
| Total Percent Similarity: | 100.00 | Total Percent Identity: | 100.00 |
| Gaps: | 0 | | |

Alignment:

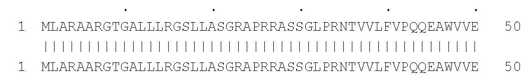

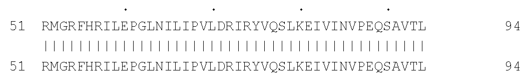

### Description for Cluster R11723

Cluster R11723 features 6 transcript(s) and 26 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| R11723_PEA_1_T15 | 75 |
| R11723_PEA_1_T17 | 76 |
| R11723_PEA_1_T19 | 77 |
| R11723_PEA_1_T20 | 78 |
| R11723_PEA_1_T5 | 79 |
| R11723_PEA_1_T6 | 80 |

TABLE 2
Segments of interest

| Segment Name | SEQ ID NO: |
|---|---|
| R11723_PEA_1_node_13 | 450 |
| R11723_PEA_1_node_16 | 451 |
| R11723_PEA_1_node_19 | 452 |
| R11723_PEA_1_node_2 | 453 |
| R11723_PEA_1_node_22 | 454 |
| R11723_PEA_1_node_31 | 455 |
| R11723_PEA_1_node_10 | 456 |
| R11723_PEA_1_node_11 | 457 |
| R11723_PEA_1_node_15 | 458 |
| R11723_PEA_1_node_18 | 459 |
| R11723_PEA_1_node_20 | 460 |
| R11723_PEA_1_node_21 | 461 |
| R11723_PEA_1_node_23 | 462 |
| R11723_PEA_1_node_24 | 463 |
| R11723_PEA_1_node_25 | 464 |
| R11723_PEA_1_node_26 | 465 |
| R11723_PEA_1_node_27 | 466 |
| R11723_PEA_1_node_28 | 467 |
| R11723_PEA_1_node_29 | 468 |
| R11723_PEA_1_node_3 | 469 |
| R11723_PEA_1_node_30 | 470 |
| R11723_PEA_1_node_4 | 471 |
| R11723_PEA_1_node_5 | 472 |
| R11723_PEA_1_node_6 | 473 |
| R11723_PEA_1_node_7 | 474 |
| R11723_PEA_1_node_8 | 475 |

TABLE 3
Proteins of interest

| Protein Name | SEQ ID NO: |
|---|---|
| R11723_PEA_1_P2 | 603 |
| R11723_PEA_1_P6 | 604 |
| R11723_PEA_1_P7 | 605 |
| R11723_PEA_1_P13 | 606 |
| R11723_PEA_1_P10 | 607 |

Cluster R11723 can be used as a diagnostic marker according to overexpression of transcripts of this cluster in cancer. Expression of such transcripts in normal tissues is also given according to the previously described methods. The term “number” in the right hand column of the table and the numbers on the y-axis of the figure below refer to weighted expression of ESTs in each category, as “parts per million” (ratio of the expression of ESTs for a particular cluster to the expression of all ESTs in that category, according to parts per million).

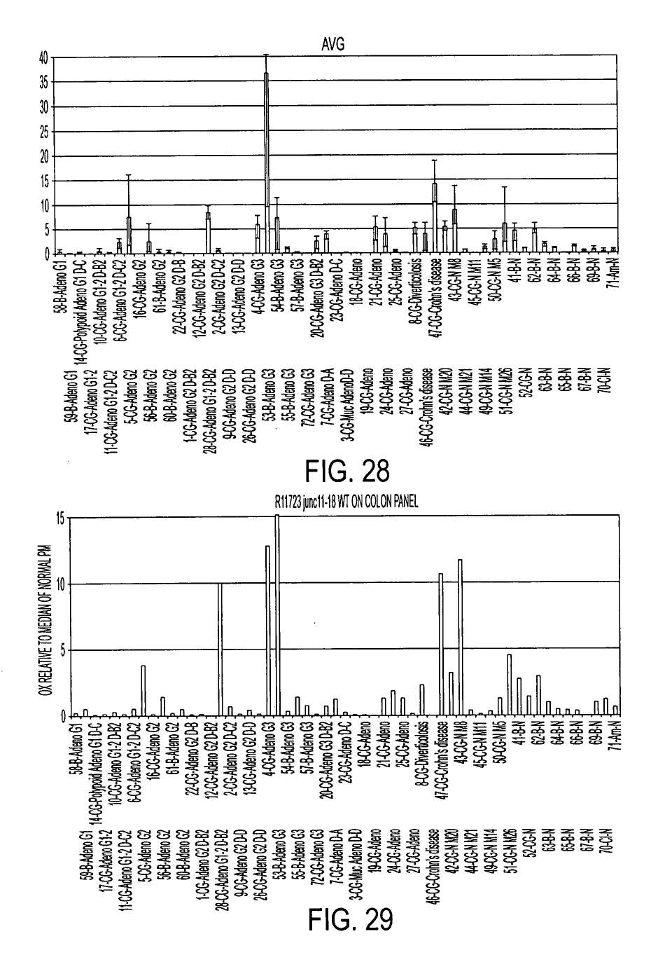

Overall, the following results were obtained as shown with regard to the histograms in FIG. 27 and Table 4. This cluster is overexpressed (at least at a minimum level) in the following pathological conditions: epithelial malignant tumors, a mixture of malignant tumors from different tissues and kidney malignant tumors.

TABLE 4
Normal tissue distribution

| Name of Tissue | Number |
|---|---|
| adrenal | 0 |
| brain | 30 |
| epithelial | 3 |
| general | 17 |
| Head and neck | 0 |
| kidney | 0 |
| Lung | 0 |
| breast | 0 |
| ovary | 0 |
| pancreas | 10 |
| skin | 0 |
| uterus | 0 |

TABLE 5
P values and ratios for expression in cancerous tissue

| Name of Tissue | P1 | P2 | SP1 | R3 | SP2 | R4 |
|---|---|---|---|---|---|---|
| adrenal | 4.2e−01 | 4.6e−01 | 4.6e−01 | 2.2 | 5.3e−01 | 1.9 |
| brain | 2.2e−01 | 2.0e−01 | 1.2e−02 | 2.8 | 5.0e−02 | 2.0 |
| epithelial | 3.0e−05 | 6.3e−05 | 1.8e−05 | 6.3 | 3.4e−06 | 6.4 |
| general | 7.2e−03 | 4.0e−02 | 1.3e−04 | 2.1 | 1.1e−03 | 1.7 |
| head and neck | 1 | 5.0e−01 | 1 | 1.0 | 7.5e−01 | 1.3 |
| kidney | 1.5e−01 | 2.4e−01 | 4.4e−03 | 5.4 | 2.8e−02 | 3.6 |
| lung | 1.2e−01 | 1.6e−01 | 1 | 1.6 | 1 | 1.3 |
| breast | 5.9e−01 | 4.4e−01 | 1 | 1.1 | 6.8e−01 | 1.5 |
| ovary | 1.6e−02 | 1.3e−02 | 1.0e−01 | 3.8 | 7.0e−02 | 3.5 |
| pancreas | 5.5e−01 | 2.0e−01 | 3.9e−01 | 1.9 | 1.4e−01 | 2.7 |
| skin | 1 | 4.4e−01 | 1 | 1.0 | 1.9e−02 | 2.1 |
| uterus | 1.5e−02 | 5.4e−02 | 1.9e−01 | 3.1 | 1.4e−01 | 2.5 |

As noted above, cluster R11723 features 6 transcript(s), which were listed in Table 1 above. A description of each variant protein according to the present invention is now provided.

Variant protein R11723_PEA_{—}1_P2 (SEQ ID NO:603) according to the present invention has an amino acid sequence as given at the end of the application; it is encoded by transcript(s) R11723_PEA_{—}1_T6 (SEQ ID NO:80). The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein R11723_PEA_{—}1_P2 (SEQ ID NO:603) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein R11723_PEA_{—}1_P2 (SEQ ID NO:603) sequence provides support for the deduced sequence of this variant protein according to the present invention).

TABLE 6
Amino acid mutations

| SNP position(s) on amino acid | Alternative | |
|---|---|---|
| sequence | amino acid(s) | Previously known SNP? |
| 107 | H -> P | Yes |
| 70 | G -> | No |
| 70 | G -> C | No |

Variant protein R11723_PEA_{—}1_P2 (SEQ ID NO:603) is encoded by the following transcript(s): R11723_PEA_{—}1_T6 (SEQ ID NO:80), for which the sequence(s) is/are given at the end of the application. The coding portion of transcript R11723_PEA_{—}1_T6 (SEQ ID NO:80) is shown in bold; this coding portion starts at position 1716 and ends at position 2051. The transcript also has the following SNPs as listed in Table 7 (given according to their position on the nucleotide sequence, with the alternative nucleic acid listed; the last column indicates whether the SNP is known or not; the presence of known SNPs in variant protein R11723_PEA_{—}1_P2 (SEQ ID NO:603) sequence provides support for the deduced sequence of this variant protein according to the present invention).

TABLE 7
Nucleic acid SNPs

| SNP position on necleotide | Alternative | |
|---|---|---|
| sequence | nucleic acid | Previously known SNP? |
| 1231 | C -> T | Yes |
| 1278 | G -> C | Yes |
| 1923 | G -> | No |
| 1923 | G -> T | No |
| 2035 | A -> C | Yes |
| 2048 | A -> C | No |
| 2057 | A -> G | Yes |

Variant protein R11723_PEA_{—}1_P6 (SEQ ID NO:604) according to the present invention has an amino acid sequence as given at the end of the application; it is encoded by transcript(s) R11723_PEA_{—}1_T15 (SEQ ID NO:75). One or more alignments to one or more previously published protein sequences are given at the end of the application. A brief description of the relationship of the variant protein according to the present invention to each such aligned protein is as follows:

Comparison Report Between R11723_PEA_{—}1_P6 (SEQ ID NO:604) and Q8IXM0 (SEQ ID NO:1393):

1. An isolated chimeric polypeptide encoding for R11723_PEA_{—}1_P6 (SEQ ID NO:604), comprising a first amino acid sequence being at least 70%, optionally at least 80%, preferably at least 85%, more preferably at least 90% and most preferably at least 95% homologous to a polypeptide having the sequence MWVLGIAATFCGLFLLPGFALQIQCYQCEEFQLNNDCSSPEFIVNCTVNVQDMCQKEVMEQSAGIMYRKS CASSAACLIASAGSPCRGLAPGREEQRALHKAGAVGGGVR (SEQ ID NO:1558) corresponding to amino acids 1-110 of R11723_PEA_{—}1_P6 (SEQ ID NO:604), and a second amino acid sequence being at least 90% homologous to MYAQALLVVGVLQRQAAAQHLHEHPPKLLRGHRVQERVDDRAEVEKRLREGEEDHVRPEVGPRPVVLG FGRSHDPPNLVGHPAYGQCHNNQPWADTSRRERQRKEKHSMRTQ corresponding to amino acids 1-112 of Q8IXM0, which also corresponds to amino acids 111-222 of R11723_PEA_{—}1_P6 (SEQ ID NO:604), wherein said first and second amino acid sequences are contiguous and in a sequential order.

2. An isolated polypeptide encoding for a head of R11723_PEA_{—}1_P6 (SEQ ID NO:604), comprising a polypeptide being at least 70%, optionally at least about 80%, preferably at least about 85%, more preferably at least about 90% and most preferably at least about 95% homologous to the sequence MWVLGIAATFCGLFLLPGFALQIQCYQCEEFQLNNDCSSPEFIVNCTVNVQDMCQKEVMEQSAGIMYRKS CASSAACLIASAGSPCRGLAPGREEQRALHKAGAVGGGVR (SEQ ID NO:1558) of R11723_PEA_{—}1_P6 (SEQ ID NO:604).

### Comparison Report Between R11723_PEA_{—}1_P6 (SEQ ID NO:604) and Q96AC2 (SEQ ID NO:1394):

1. An isolated chimeric polypeptide encoding for R11723_PEA_{—}1_P6 (SEQ ID NO:604), comprising a first amino acid sequence being at least 90% homologous to MWVLGIAATFCGLFLLPGFALQIQCYQCEEFQLNNDCSSPEFIVNCTVNVQDMCQKEVMEQSAGIMYRKS CASSAACLIASAG corresponding to amino acids 1-83 of Q96AC2, which also corresponds to amino acids 1-83 of R11723_PEA_{—}1_P6 (SEQ ID NO:604), and a second amino acid sequence being at least 70%, optionally at least 80%, preferably at least 85%, more preferably at least 90% and most preferably at least 95% homologous to a polypeptide having the sequence SPCRGLAPGREEQRALHKAGAVGGGVRMYAQALLVVGVLQRQAAAQHLHEHPPKLLRGHRVQERVDD RAEVEKRLREGEEDHVRPEVGPRPVVLGFGRSHDPPNLVGHPAYGQCHNNQPWADTSRRERQRKEKHSM RTQ (SEQ ID NO:1559) corresponding to amino acids 84-222 of R11723_PEA_{—}1_P6 (SEQ ID NO:604) wherein said first and second amino acid sequences are contiguous and in a sequential order.

2. An isolated polypeptide encoding for a tail of R11723_PEA_{—}1_P6 (SEQ ID NO:604), comprising a polypeptide being at least 70%, optionally at least about 80%, preferably at least about 85%, more preferably at least about 90% and most preferably at least about 95% homologous to the sequence SPCRGLAPGREEQRALHKAGAVGGGVRMYAQALLVVGVLQRQAAAQHLHEHPPKLLRGHRVQERVDD RAEVEKRLREGEEDHVRPEVGPRPVVLGFGRSHDPPNLVGHPAYGQCHNNQPWADTSRRERQRKEKHSM RTQ SEQ ID NO:1559) in R11723_PEA_{—}1_P6 (SEQ ID NO:604).

Comparison Report Between R11723_PEA_{—}1_P6 (SEQ ID NO:604) and Q8N2G4 (SEQ ID NO:1395):

1. An isolated chimeric polypeptide encoding for R11723_PEA_{—}1_P6 (SEQ ID NO:604), comprising a first amino acid sequence being at least 90% homologous to MWVLGIAATFCGLFLLPGFALQIQCYQCEEFQLNNDCSSPEFIVNCTVNVQDMCQKEVMEQSAGIMYRKS CASSAACLIASAG corresponding to amino acids 1-83 of Q8N2G4, which also corresponds to amino acids 1-83 of R11723_PEA_{—}1_P6 (SEQ ID NO:604), and a second amino acid sequence being at least 70%, optionally at least 80%, preferably at least 85%, more preferably at least 90% and most preferably at least 95% homologous to a polypeptide having the sequence SPCRGLAPGREEQRALHKAGAVGGGVRMYAQALLVVGVLQRQAAAQHLHEHPPKLLRGHRVQERVDD RAEVEKRLREGEEDHVRPEVGPRPVVLGFGRSHDPPNLVGHPAYGQCHNNQPWADTSRRERQRKEKHSM RTQ SEQ ID NO:1559) corresponding to amino acids 84-222 of R11723_PEA_{—}1_P6 (SEQ ID NO:604), wherein said first and second amino acid sequences are contiguous and in a sequential order.

2. An isolated polypeptide encoding for a tail of R11723_PEA_{—}1_P6 (SEQ ID NO:604), comprising a polypeptide being at least 70%, optionally at least about 80%, preferably at least about 85%, more preferably at least about 90% and most preferably at least about 95% homologous to the sequence SPCRGLAPGREEQRALHKAGAVGGGVRMYAQALLVVGVLQRQAAAQHLHEHPPKLLRGHRVQERVDD RAEVEKRLREGEEDHVRPEVGPRPVVLGFGRSHDPPNLVGHPAYGQCHNNQPWADTSRRERQRKEKHSM RTQ SEQ ID NO:1559) in R11723_PEA_{—}1_P6 (SEQ ID NO:604).

### Comparison Report Between R11723_PEA_{—}1_P6 (SEQ ID NO:604) and BAC85518 (SEQ ID NO:1396):

1. An isolated chimeric polypeptide encoding for R11723_PEA_{—}1_P6 (SEQ ID NO:604), comprising a first amino acid sequence being at least 90% homologous to MWVLGIAATFCGLFLLPGFALQIQCYQCEEFQLNNDCSSPEFIVNCTVNVQDMCQKEVMEQSAGIMYRKS CASSAACLIASAG corresponding to amino acids 24-106 of BAC85518 (SEQ ID NO:1396), which also corresponds to amino acids 1-83 of R11723_PEA_{—}1_P6 (SEQ ID NO:604), and a second amino acid sequence being at least 70%, optionally at least 80%, preferably at least 85%, more preferably at least 90% and most preferably at least 95% homologous to a polypeptide having the sequence SPCRGLAPGREEQRALHKAGAVGGGVRMYAQALLVVGVLQRQAAAQHLHEHPPKLLRGHRVQERVDD RAEVEKRLREGEEDHVRPEVGPRPVVLGFGRSHDPPNLVGHPAYGQCHNNQPWADTSRRERQRKEKHSM RTQ SEQ ID NO:1559) corresponding to amino acids 84-222 of R11723_PEA_{—}1_P6 (SEQ ID NO:604), wherein said first and second amino acid sequences are contiguous and in a sequential order.

2. An isolated polypeptide encoding for a tail of R11723_PEA_{—}1_P6 (SEQ ID NO:604), comprising a polypeptide being at least 70%, optionally at least about 80%, preferably at least about 85%, more preferably at least about 90% and most preferably at least about 95% homologous to the sequence SPCRGLAPGREEQRALHKAGAVGGGVRMYAQALLVVGVLQRQAAAQHLHEHPPKLLRGHRVQERVDD RAEVEKRLREGEEDHVRPEVGPRPVVLGFGRSHDPPNLVGHPAYGQCHNNQPWADTSRRERQRKEKHSM RTQ SEQ ID NO:1559) in R11723_PEA_{—}1_P6 (SEQ ID NO:604).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein R11723_PEA_{—}1_P6 (SEQ ID NO:604) also has the following non-silent SNPs (Single Nucleotide Polymorphisms) as listed in Table 8, (given according to their position(s) on the amino acid sequence, with the alternative amino acid(s) listed; the last column indicates whether the SNP is known or not; the presence of known SNPs in variant protein R11723_PEA_{—}1_P6 (SEQ ID NO:604) sequence provides support for the deduced sequence of this variant protein according to the present invention).

TABLE 8
Amino acid mutations

| SNP position(s) on amino acid | Alternative | |
|---|---|---|
| sequence | amino acid(s) | Previously known SNP? |
| 180 | G -> | No |
| 180 | G -> C | No |
| 217 | H -> P | Yes |

Variant protein R11723_PEA_{—}1_P6 (SEQ ID NO:604) is encoded by the following transcript(s): R11723_PEA_{—}1_T15 (SEQ ID NO:75), for which the sequence(s) is/are given at the end of the application. The coding portion of transcript R11723_PEA_{—}1_T15 (SEQ ID NO:75) is shown in bold; this coding portion starts at position 434 and ends at position 1099. The transcript also has the following SNPs as listed in Table 9 (given according to their position on the nucleotide sequence, with the alternative nucleic acid listed; the last column indicates whether the SNP is known or not; the presence of known SNPs in variant protein R11723_PEA_{—}1_P6 (SEQ ID NO:604) sequence provides support for the deduced sequence of this variant protein according to the present invention).

TABLE 9
Nucleic acid SNPs

| SNP position on nucleotide | Alternative | |
|---|---|---|
| sequence | nucleic acid | Previously known SNP? |
| 971 | G -> | No |
| 971 | G -> T | No |
| 1083 | A -> C | Yes |
| 1096 | A -> C | No |
| 1105 | A -> G | Yes |

Variant protein R11723_PEA_{—}1_P7 (SEQ ID NO:605) according to the present invention has an amino acid sequence as given at the end of the application; it is encoded by transcript(s) R11723_PEA_{—}1_T17 (SEQ ID NO:76). One or more alignments to one or more previously published protein sequences are given at the end of the application. A brief description of the relationship of the variant protein according to the present invention to each such aligned protein is as follows:

Comparison Report Between R11723_PEA_{—}1_P7 (SEQ ID NO:605) and Q96AC2 (SEQ ID NO: 1394):

1. An isolated chimeric polypeptide encoding for R11723_PEA_{—}1_P7 (SEQ ID NO:605), comprising a first amino acid sequence being at least 90% homologous to MWVLGIAATFCGLFLLPGFALQIQCYQCEEFQLNNDCSSPEFIVNCTVNVQDMCQKEVMEQSAG corresponding to amino acids 1-64 of Q96AC2 (SEQ ID NO: 1394), which also corresponds to amino acids 1-64 of R11723_PEA_{—}1_P7 (SEQ ID NO:605), and a second amino acid sequence being at least 70%, optionally at least 80%, preferably at least 85%, more preferably at least 90% and most preferably at least 95% homologous to a polypeptide having the sequence SHCVTRLECSGTISAHCNLCLPGSNDHPT (SEQ ID NO:1560) corresponding to amino acids 65-93 of R11723_PEA_{—}1_P7 (SEQ ID NO:605), wherein said first and second amino acid sequences are contiguous and in a sequential order.

2. An isolated polypeptide encoding for a tail of R11723_PEA_{—}1_P7 (SEQ ID NO:605), comprising a polypeptide being at least 70%, optionally at least about 80%, preferably at least about 85%, more preferably at least about 90% and most preferably at least about 95% homologous to the sequence SHCVTRLECSGTISAHCNLCLPGSNDHPT (SEQ ID NO:1560) in R11723_PEA_{—}1_P7 (SEQ ID NO:605).

Comparison Report Between R11723_PEA_{—}1_P7 (SEQ ID NO:605) and Q8N2G4 (SEQ ID NO: 1395):

1. An isolated chimeric polypeptide encoding for R11723_PEA_{—}1_P7 (SEQ ID NO:605), comprising a first amino acid sequence being at least 90% homologous to MWVLGIAATFCGLFLLPGFALQIQCYQCEEFQLNNDCSSPEFIVNCTVNVQDMCQKEVMEQSAG corresponding to amino acids 1-64 of Q8N2G4 (SEQ ID NO: 1395), which also corresponds to amino acids 1-64 of R11723_PEA_{—}1_P7 (SEQ ID NO:605), and a second amino acid sequence being at least 70%, optionally at least 80%, preferably at least 85%, more preferably at least 90% and most preferably at least 95% homologous to a polypeptide having the sequence SHCVTRLECSGTISAHCNLCLPGSNDHPT (SEQ ID NO:1560) corresponding to amino acids 65-93 of R11723_PEA_{—}1_P7 (SEQ ID NO:605), wherein said first and second amino acid sequences are contiguous and in a sequential order.

2. An isolated polypeptide encoding for a tail of R11723_PEA_{—}1_P7 (SEQ ID NO:605), comprising a polypeptide being at least 70%, optionally at least about 80%, preferably at least about 85%, more preferably at least about 90% and most preferably at least about 95% homologous to the sequence SHCVTRLECSGTISAHCNLCLPGSNDHPT (SEQ ID NO:1560) in R11723_PEA_{—}1_P7 (SEQ ID NO:605).

Comparison Report Between R11723_PEA_{—}1_P7 (SEQ ID NO:605) and BAC85273 (SEQ ID NO:1397):

1. An isolated chimeric polypeptide encoding for R11723_PEA_{—}1_P7 (SEQ ID NO:605), comprising a first amino acid sequence being at least 70%, optionally at least 80%, preferably at least 85%, more preferably at least 90% and most preferably at least 95% homologous to a polypeptide having the sequence MWVLG (SEQ ID NO:1561) corresponding to amino acids 1-5 of R11723_PEA_{—}1_P7 (SEQ ID NO:605), second amino acid sequence being at least 90% homologous to IAATFCGLFLLPGFALQIQCYQCEEFQLNNDCSSPEFIVNCTVNVQDMCQKEVMEQSAG corresponding to amino acids 22-80 of BAC85273 (SEQ ID NO:1397), which also corresponds to amino acids 6-64 of R11723_PEA_{—}1_P7 (SEQ ID NO:605), and a third amino acid sequence being at least 70%, optionally at least 80%, preferably at least 85%, more preferably at least 90% and most preferably at least 95% homologous to a polypeptide having the sequence SHCVTRLECSGTISAHCNLCLPGSNDHPT (SEQ ID NO:1560) corresponding to amino acids 65-93 of R11723_PEA_{—}1_P7 (SEQ ID NO:605), wherein said first, second and third amino acid sequences are contiguous and in a sequential order.

2. An isolated polypeptide encoding for a head of R11723_PEA_{—}1_P7 (SEQ ID NO:605), comprising a polypeptide being at least 70%, optionally at least about 80%, preferably at least about 85%, more preferably at least about 90% and most preferably at least about 95% homologous to the sequence MWVLG (SEQ ID NO:1561) of R11723_PEA_{—}1_P7 (SEQ ID NO:605).

3. An isolated polypeptide encoding for a tail of R11723_PEA_{—}1_P7 (SEQ ID NO:605), comprising a polypeptide being at least 70%, optionally at least about 80%, preferably at least about 85%, more preferably at least about 90% and most preferably at least about 95% homologous to the sequence SHCVTRLECSGTISAHCNLCLPGSNDHPT (SEQ ID NO:1560) in R11723_PEA_{—}1_P7 (SEQ ID NO:605).

Comparison Report Between R11723_PEA_{—}1_P7 (SEQ ID NO:605) and BAC85518 (SEQ ID NO:1396):

1. An isolated chimeric polypeptide encoding for R11723_PEA_{—}1_P7 (SEQ ID NO:605), comprising a first amino acid sequence being at least 90% homologous to MWVLGIAATFCGLFLLPGFALQIQCYQCEEFQLNNDCSSPEFIVNCTVNVQDMCQKEVMEQSAG corresponding to amino acids 24-87 of BAC85518 (SEQ ID NO:1396), which also corresponds to amino acids 1-64 of R11723_PEA_{—}1_P7 (SEQ ID NO:605), and a second amino acid sequence being at least 70%, optionally at least 80%, preferably at least 85%, more preferably at least 90% and most preferably at least 95% homologous to a polypeptide having the sequence SHCVTRLECSGTISAHCNLCLPGSNDHPT (SEQ ID NO:1560) corresponding to amino acids 65-93 of R11723_PEA_{—}1_P7 (SEQ ID NO:605), wherein said first and second amino acid sequences are contiguous and in a sequential order.

2. An isolated polypeptide encoding for a tail of R11723_PEA_{—}1_P7 (SEQ ID NO:605), comprising a polypeptide being at least 70%, optionally at least about 80%, preferably at least about 85%, more preferably at least about 90% and most preferably at least about 95% homologous to the sequence SHCVTRLECSGTISAHCNLCLPGSNDHPT (SEQ ID NO:1560) in R11723_PEA_{—}1_P7 (SEQ ID NO:605).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein R11723_PEA_{—}1_P7 (SEQ ID NO:605) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein R11723_PEA_{—}1_P7 (SEQ ID NO:605) sequence provides support for the deduced sequence of this variant protein according to the present invention).

TABLE 10
Amino acid mutations

| SNP position(s) on amino acid | Alternative | |
|---|---|---|
| sequence | amino acid(s) | Previously known SNP? |
| 67 | C -> S | Yes |

Variant protein R11723_PEA_{—}1_P7 (SEQ ID NO:605) is encoded by the following transcript(s): R11723_PEA_{—}1_T17 (SEQ ID NO:76), for which the sequence(s) is/are given at the end of the application. The coding portion of transcript R11723_PEA_{—}1_T17 (SEQ ID NO:76) is shown in bold; this coding portion starts at position 434 and ends at position 712. The transcript also has the following SNPs as listed in Table 11 (given according to their position on the nucleotide sequence, with the alternative nucleic acid listed; the last column indicates whether the SNP is known or not; the presence of known SNPs in variant protein R11723_PEA_{—}1_P7 (SEQ ID NO:605) sequence provides support for the deduced sequence of this variant protein according to the present invention).

TABLE 11
Nucleic acid SNPs

| SNP position on nucleotide | Alternative | |
|---|---|---|
| sequence | nucleic acid | Previously known SNP? |
| 625 | G -> T | Yes |
| 633 | G -> C | Yes |
| 1303 | C -> T | Yes |

Variant protein R11723_PEA_{—}1_P13 (SEQ ID NO:606) according to the present invention has an amino acid sequence as given at the end of the application; it is encoded by transcript(s) R11723_PEA_{—}1_T19 (SEQ ID NO:77) and R11723_PEA_{—}1_T5 (SEQ ID NO:79). One or more alignments to one or more previously published protein sequences are given at the end of the application. A brief description of the relationship of the variant protein according to the present invention to each such aligned protein is as follows:

Comparison Report Between R11723_PEA_{—}1_P13 (SEQ ID NO:606) and Q96AC2 (SEQ ID NO: 1394):

1. An isolated chimeric polypeptide encoding for R11723_PEA_{—}1_P13 (SEQ ID NO:606), comprising a first amino acid sequence being at least 90% homologous to MWVLGIAATFCGLFLLPGFALQIQCYQCEEFQLNNDCSSPEFIVNCTVNVQDMCQKEVMEQSA corresponding to amino acids 1-63 of Q96AC2 (SEQ ID NO: 1394), which also corresponds to amino acids 1-63 of R11723_PEA_{—}1_P13 (SEQ ID NO:606), and a second amino acid sequence being at least 70%, optionally at least 80%, preferably at least 85%, more preferably at least 90% and most preferably at least 95% homologous to a polypeptide having the sequence DTKRTNTLLFEMRHFAKQLTT (SEQ ID NO:1562) corresponding to amino acids 64-84 of R11723_PEA_{—}1_P13 (SEQ ID NO:606), wherein said first and second amino acid sequences are contiguous and in a sequential order.

2. An isolated polypeptide encoding for a tail of R11723_PEA_{—}1_P13 (SEQ ID NO:606), comprising a polypeptide being at least 70%, optionally at least about 80%, preferably at least about 85%, more preferably at least about 90% and most preferably at least about 95% homologous to the sequence DTKRTNTLLFEMRHFAKQLTT (SEQ ID NO:1562) in R11723_PEA_{—}1_P13 (SEQ ID NO:606).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein R11723_PEA_{—}1_P13 (SEQ ID NO:606) is encoded by the following transcript(s): R11723_PEA_{—}1_T19 (SEQ ID NO:77), for which the sequence(s) is/are given at the end of the application. The coding portion of transcript R11723_PEA_{—}1_T19 (SEQ ID NO:77) is shown in bold; this coding portion starts at position 434 and ends at position 685. The transcript also has the following SNPs as listed in Table 12 (given according to their position on the nucleotide sequence, with the alternative nucleic acid listed; the last column indicates whether the SNP is known or not; the presence of known SNPs in variant protein R11723_PEA_{—}1_P13 (SEQ ID NO:606) sequence provides support for the deduced sequence of this variant protein according to the present invention).

TABLE 12
Nucleic acid SNPs

| SNP position on nucleotide | Alternative | |
|---|---|---|
| sequence | nucleic acid | Previously known SNP? |
| 778 | G -> T | Yes |
| 786 | G -> C | Yes |
| 1456 | C -> T | Yes |

Variant protein R11723_PEA_{—}1_P10 (SEQ ID NO:607) according to the present invention has an amino acid sequence as given at the end of the application; it is encoded by transcript(s) R11723_PEA_{—}1_T20 (SEQ ID NO:78). One or more alignments to one or more previously published protein sequences are given at the end of the application. A brief description of the relationship of the variant protein according to the present invention to each such aligned protein is as follows:

Comparison Report Between R11723_PEA_{—}1_P10 (SEQ ID NO:607) and Q96AC2 (SEQ ID NO: 1394):

1. An isolated chimeric polypeptide encoding for R11723_PEA_{—}1_P10 (SEQ ID NO:607), comprising a first amino acid sequence being at least 90% homologous to MWVLGIAATFCGLFLLPGFALQIQCYQCEEFQLNNDCSSPEFIVNCTVNVQDMCQKEVMEQSA corresponding to amino acids 1-63 of Q96AC2 (SEQ ID NO: 1394), which also corresponds to amino acids 1-63 of R11723_PEA_{—}1_P10 (SEQ ID NO:607), and a second amino acid sequence being at least 70%, optionally at least 80%, preferably at least 85%, more preferably at least 90% and most preferably at least 95% homologous to a polypeptide having the sequence DRVSLCHEAGVQWNNFSTLQPLPPRLK (SEQ ID NO:1563) corresponding to amino acids 64-90 of R11723_PEA_{—}1_P10 (SEQ ID NO:607), wherein said first and second amino acid sequences are contiguous and in a sequential order.

2. An isolated polypeptide encoding for a tail of R11723_PEA_{—}1_P10 (SEQ ID NO:607), comprising a polypeptide being at least 70%, optionally at least about 80%, preferably at least about 85%, more preferably at least about 90% and most preferably at least about 95% homologous to the sequence DRVSLCHEAGVQWNNFSTLQPLPPRLK (SEQ ID NO:1563) in R11723_PEA_{—}1_P10 (SEQ ID NO:607).

Comparison Report Between R11723_PEA_{—}1_P10 (SEQ ID NO:607) and Q8N2G4 (SEQ ID NO: 1395):

1. An isolated chimeric polypeptide encoding for R11723_PEA_{—}1_P10 (SEQ ID NO:607), comprising a first amino acid sequence being at least 90% homologous to MWVLGIAATFCGLFLLPGFALQIQCYQCEEFQLNNDCSSPEFIVNCTVNVQDMCQKEVMEQSA corresponding to amino acids 1-63 of Q8N2G4 (SEQ ID NO: 1395), which also corresponds to amino acids 1-63 of R11723_PEA_{—}1_P10 (SEQ ID NO:607), and a second amino acid sequence being at least 70%, optionally at least 80%, preferably at least 85%, more preferably at least 90% and most preferably at least 95% homologous to a polypeptide having the sequence DRVSLCHEAGVQWNNFSTLQPLPPRLK (SEQ ID NO:1563) corresponding to amino acids 64-90 of R11723_PEA_{—}1_P10 (SEQ ID NO:607), wherein said first and second amino acid sequences are contiguous and in a sequential order.

2. An isolated polypeptide encoding for a tail of R11723_PEA_{—}1_P10 (SEQ ID NO:607), comprising a polypeptide being at least 70%, optionally at least about 80%, preferably at least about 85%, more preferably at least about 90% and most preferably at least about 95% homologous to the sequence DRVSLCHEAGVQWNNFSTLQPLPPRLK (SEQ ID NO:1563) in R11723_PEA_{—}1_P10 (SEQ ID NO:607).

### Comparison Report Between R11723_PEA_{—}1_P10 (SEQ ID NO:607) and BAC85273 (SEQ ID NO:1397) (SEQ ID NO:1397):

1. An isolated chimeric polypeptide encoding for R11723_PEA_{—}1_P10 (SEQ ID NO:607), comprising a first amino acid sequence being at least 70%, optionally at least 80%, preferably at least 85%, more preferably at least 90% and most preferably at least 95% homologous to a polypeptide having the sequence MWVLG (SEQ ID NO:1561) corresponding to amino acids 1-5 of R11723_PEA_{—}1_P10 (SEQ ID NO:607), second amino acid sequence being at least 90% homologous to IAATFCGLFLLPGFALQIQCYQCEEFQLNNDCSSPEFIVNCTVNVQDMCQKEVMEQSA corresponding to amino acids 22-79 of BAC85273 (SEQ ID NO:1397), which also corresponds to amino acids 6-63 of R11723_PEA_{—}1_P10 (SEQ ID NO:607), and a third amino acid sequence being at least 70%, optionally at least 80%, preferably at least 85%, more preferably at least 90% and most preferably at least 95% homologous to a polypeptide having the sequence DRVSLCHEAGVQWNNFSTLQPLPPRLK (SEQ ID NO:1563) corresponding to amino acids 64-90 of R11723_PEA_{—}1_P10 (SEQ ID NO:607), wherein said first, second and third amino acid sequences are contiguous and in a sequential order.

2. An isolated polypeptide encoding for a head of R11723_PEA_{—}1_P10 (SEQ ID NO:607), comprising a polypeptide being at least 70%, optionally at least about 80%, preferably at least about 85%, more preferably at least about 90% and most preferably at least about 95% homologous to the sequence MWVLG (SEQ ID NO:1561) of R11723_PEA_{—}1_P10 (SEQ ID NO:607).

3. An isolated polypeptide encoding for a tail of R11723_PEA_{—}1_P10 (SEQ ID NO:607), comprising a polypeptide being at least 70%, optionally at least about 80%, preferably at least about 85%, more preferably at least about 90% and most preferably at least about 95% homologous to the sequence DRVSLCHEAGVQWNNFSTLQPLPPRLK (SEQ ID NO:1563) in R11723_PEA_{—}1_P10 (SEQ ID NO:607).

Comparison Report Between R11723_PEA_{—}1_P10 (SEQ ID NO:607) and BAC85518 (SEQ ID NO:1396):

1. An isolated chimeric polypeptide encoding for R11723_PEA_{—}1_P10 (SEQ ID NO:607), comprising a first amino acid sequence being at least 90% homologous to MWVLGIAATFCGLFLLPGFALQIQCYQCEEFQLNNDCSSPEFIVNCTVNVQDMCQKEVMEQSA corresponding to amino acids 24-86 of BAC85518 (SEQ ID NO:1396), which also corresponds to amino acids 1-63 of R11723_PEA_{—}1_P10 (SEQ ID NO:607), and a second amino acid sequence being at least 70%, optionally at least 80%, preferably at least 85%, more preferably at least 90% and most preferably at least 95% homologous to a polypeptide having the sequence DRVSLCHEAGVQWNNFSTLQPLPPRLK (SEQ ID NO:1563) corresponding to amino acids 64-90 of R11723_PEA_{—}1_P10 (SEQ ID NO:607), wherein said first and second amino acid sequences are contiguous and in a sequential order.

2. An isolated polypeptide encoding for a tail of R11723_PEA_{—}1_P10 (SEQ ID NO:607), comprising a polypeptide being at least 70%, optionally at least about 80%, preferably at least about 85%, more preferably at least about 90% and most preferably at least about 95% homologous to the sequence DRVSLCHEAGVQWNNFSTLQPLPPRLK (SEQ ID NO:1563) in R11723_PEA_{—}1_P10 (SEQ ID NO:607).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein R11723_PEA_{—}1_P10 (SEQ ID NO:607) also has the following non-silent SNPs (Single Nucleotide Polymorphisms) as listed in Table 13, (given according to their position(s) on the amino acid sequence, with the alternative amino acid(s) listed; the last column indicates whether the SNP is known or not; the presence of known SNPs in variant protein R11723_PEA_{—}1_P10 (SEQ ID NO:607) sequence provides support for the deduced sequence of this variant protein according to the present invention).

TABLE 13
Amino acid mutations

| SNP position(s) on amino acid | Alternative | |
|---|---|---|
| sequence | amino acid(s) | Previously known SNP? |
| 66 | V -> F | Yes |

Variant protein R11723_PEA_{—}1_P10 (SEQ ID NO:607) is encoded by the following transcript(s): R11723_PEA_{—}1_T20 (SEQ ID NO:78), for which the sequence(s) is/are given at the end of the application. The coding portion of transcript R11723_PEA_{—}1_T20 (SEQ ID NO:78) is shown in bold; this coding portion starts at position 434 and ends at position 703. The transcript also has the following SNPs as listed in Table 14 (given according to their position on the nucleotide sequence, with the alternative nucleic acid listed; the last column indicates whether the SNP is known or not; the presence of known SNPs in variant protein R11723_PEA_{—}1_P10 (SEQ ID NO:607) sequence provides support for the deduced sequence of this variant protein according to the present invention).

TABLE 14
Nucleic acid SNPs

| SNP position on nucleotide | Alternative | |
|---|---|---|
| sequence | nucleic acid | Previously known SNP? |
| 629 | G -> T | Yes |
| 637 | G -> C | Yes |
| 1307 | C -> T | Yes |

As noted above, cluster R11723 features 26 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster R11723_PEA_{—}1_node_{—}13 (SEQ ID NO:450) according to the present invention is supported by 5 libraries. The number of libraries was determined as previously described. This segment can be found in the following transcript(s): R11723_PEA_{—}1_T19 (SEQ ID NO:77), R11723_PEA_{—}1_T5 (SEQ ID NO:79) and R11723_PEA_{—}1_T6 (SEQ ID NO:80). Table 15 below describes the starting and ending position of this segment on each transcript.

TABLE 15
Segment location on transcripts

| | | Segment |
|---|---|---|
| | Segment | ending |
| Transcript name | starting position | position |
| R11723_PEA_1_T19 (SEQ ID NO: 77) | 624 | 776 |
| R11723_PEA_1_T5 (SEQ ID NO: 79) | 624 | 776 |
| R11723_PEA_1_T6 (SEQ ID NO: 80) | 658 | 810 |

Segment cluster R11723_PEA_{—}1_node_{—}16 (SEQ ID NO:451) according to the present invention is supported by 3 libraries. The number of libraries was determined as previously described. This segment can be found in the following transcript(s): R11723_PEA_{—}1_T17 (SEQ ID NO:76), R11723_PEA_{—}1_T19 (SEQ ID NO:77) and R11723_PEA_{—}1_T20 (SEQ ID NO:78). Table 16 below describes the starting and ending position of this segment on each transcript.

TABLE 16
Segment location on transcripts

| | | Segment |
|---|---|---|
| | Segment | ending |
| Transcript name | starting position | position |
| R11723_PEA_1_T17 (SEQ ID NO: 76) | 624 | 1367 |
| R11723_PEA_1_T19 (SEQ ID NO: 77) | 777 | 1520 |
| R11723_PEA_1_T20 (SEQ ID NO: 78) | 628 | 1371 |

Segment cluster R11723_PEA_{—}1_node_{—}19 (SEQ ID NO:452) according to the present invention is supported by 45 libraries. The number of libraries was determined as previously described. This segment can be found in the following transcript(s): R11723_PEA_{—}1_T5 (SEQ ID NO:79) and R11723_PEA_{—}1_T6 (SEQ ID NO:80). Table 17 below describes the starting and ending position of this segment on each transcript.

TABLE 17
Segment location on transcripts

| | | Segment |
|---|---|---|
| | Segment | ending |
| Transcript name | starting position | position |
| R11723_PEA_1_T5 (SEQ ID NO: 79) | 835 | 1008 |
| R11723_PEA_1_T6 (SEQ ID NO: 80) | 869 | 1042 |

Segment cluster R11723_PEA_{—}1_node_{—}2 (SEQ ID NO:453) according to the present invention is supported by 29 libraries. The number of libraries was determined as previously described. This segment can be found in the following transcript(s): R11723_PEA_{—}1_T15 (SEQ ID NO:75), R11723_PEA_{—}1_T17 (SEQ ID NO:76), R11723_PEA_{—}1_T19 (SEQ ID NO:77), R11723_PEA_{—}1_T20 (SEQ ID NO:78), R11723_PEA_{—}1_T5 (SEQ ID NO:79) and R11723_PEA_{—}1_T6 (SEQ ID NO:80). Table 18 below describes the starting and ending position of this segment on each transcript.

TABLE 18
Segment location on transcripts

| | | Segment |
|---|---|---|
| | Segment | ending |
| Transcript name | starting position | position |
| R11723_PEA_1_T15 (SEQ ID NO: 75) | 1 | 309 |
| R11723_PEA_1_T17 (SEQ ID NO: 76) | 1 | 309 |
| R11723_PEA_1_T19 (SEQ ID NO: 77) | 1 | 309 |
| R11723_PEA_1_T20 (SEQ ID NO: 78) | 1 | 309 |
| R11723_PEA_1_T5 (SEQ ID NO: 79) | 1 | 309 |
| R11723_PEA_1_T6 (SEQ ID NO: 80) | 1 | 309 |

Segment cluster R11723_PEA_{—}1_node_{—}22 (SEQ ID NO:454) according to the present invention is supported by 65 libraries. The number of libraries was determined as previously described. This segment can be found in the following transcript(s): R11723_PEA_{—}1_T5 (SEQ ID NO:79) and R11723_PEA_{—}1_T6 (SEQ ID NO:80). Table 19 below describes the starting and ending position of this segment on each transcript.

TABLE 19
Segment location on transcripts

| | | Segment |
|---|---|---|
| | Segment | ending |
| Transcript name | starting position | position |
| R11723_PEA_1_T5 (SEQ ID NO: 79) | 1083 | 1569 |
| R11723_PEA_1_T6 (SEQ ID NO: 80) | 1117 | 1603 |

Segment cluster R11723_PEA_{—}1_node_{—}31 (SEQ ID NO:455) according to the present invention is supported by 70 libraries. The number of libraries was determined as previously described. This segment can be found in the following transcript(s): R11723_PEA_{—}1_T15 (SEQ ID NO:75), R11723_PEA_{—}1_T5 (SEQ ID NO:79) and R11723_PEA_{—}1_T6 (SEQ ID NO:80). Table 20 below describes the starting and ending position of this segment on each transcript (it should be noted that these transcripts show alternative polyadenylation).

TABLE 20
Segment location on transcripts

| | | Segment |
|---|---|---|
| | Segment | ending |
| Transcript name | starting position | position |
| R11723_PEA_1_T15 (SEQ ID NO: 75) | 1060 | 1295 |
| R11723_PEA_1_T5 (SEQ ID NO: 79) | 1978 | 2213 |
| R11723_PEA_1_T6 (SEQ ID NO: 80) | 2012 | 2247 |

According to an optional embodiment of the present invention, short segments related to the above cluster are also provided. These segments are up to about 120 bp in length, and so are included in a separate description.

Segment cluster R11723_PEA_{—}1_node_{—}10 (SEQ ID NO:456) according to the present invention is supported by 38 libraries. The number of libraries was determined as previously described. This segment can be found in the following transcript(s): R11723_PEA_{—}1_T15 (SEQ ID NO:75), R11723_PEA_{—}1_T17 (SEQ ID NO:76), R11723_PEA_{—}1_T19 (SEQ ID NO:77), R11723_PEA_{—}1_T20 (SEQ ID NO:78), R11723_PEA_{—}1_T5 (SEQ ID NO:79) and R11723_PEA_{—}1_T6 (SEQ ID NO:80). Table 21 below describes the starting and ending position of this segment on each transcript.

TABLE 21
Segment location on transcripts

| | | Segment |
|---|---|---|
| | Segment | ending |
| Transcript name | starting position | position |
| R11723_PEA_1_T15 (SEQ ID NO: 75) | 486 | 529 |
| R11723_PEA_1_T17 (SEQ ID NO: 76) | 486 | 529 |
| R11723_PEA_1_T19 (SEQ ID NO: 77) | 486 | 529 |
| R11723_PEA_1_T20 (SEQ ID NO: 78) | 486 | 529 |
| R11723_PEA_1_T5 (SEQ ID NO: 79) | 486 | 529 |
| R11723_PEA_1_T6 (SEQ ID NO: 80) | 520 | 563 |

Segment cluster R11723_PEA_{—}1_node_{—}11 (SEQ ID NO:457) according to the present invention is supported by 42 libraries. The number of libraries was determined as previously described. This segment can be found in the following transcript(s): R11723_PEA_{—}1_T15 (SEQ ID NO:75), R11723_PEA_{—}1_T17 (SEQ ID NO:76), R11723_PEA_{—}1_T19 (SEQ ID NO:77), R11723_PEA_{—}1_T20 (SEQ ID NO:78), R11723_PEA_{—}1_T5 (SEQ ID NO:79) and R11723_PEA_{—}1_T6 (SEQ ID NO:80). Table 22 below describes the starting and ending position of this segment on each transcript.

TABLE 22
Segment location on transcripts

| | | Segment |
|---|---|---|
| | Segment | ending |
| Transcript name | starting position | position |
| R11723_PEA_1_T15 (SEQ ID NO: 75) | 530 | 623 |
| R11723_PEA_1_T17 (SEQ ID NO: 76) | 530 | 623 |
| R11723_PEA_1_T19 (SEQ ID NO: 77) | 530 | 623 |
| R11723_PEA_1_T20 (SEQ ID NO: 78) | 530 | 623 |
| R11723_PEA_1_T5 (SEQ ID NO: 79) | 530 | 623 |
| R11723_PEA_1_T6 (SEQ ID NO: 80) | 564 | 657 |

Segment cluster R11723_PEA_{—}1_node_{—}15 (SEQ ID NO:458) according to the present invention can be found in the following transcript(s): R11723_PEA_{—}1_T20 (SEQ ID NO:78). Table 23 below describes the starting and ending position of this segment on each transcript.

TABLE 23
Segment location on transcripts

| | | Segment |
|---|---|---|
| | Segment | ending |
| Transcript name | starting position | position |
| R11723_PEA_1_T20 (SEQ ID NO: 78) | 624 | 627 |

Segment cluster R11723_PEA_{—}1_node_{—}18 (SEQ ID NO:459) according to the present invention is supported by 40 libraries. The number of libraries was determined as previously described. This segment can be found in the following transcript(s): R11723_PEA_{—}1_T15 (SEQ ID NO:75), R11723_PEA_{—}1_T5 (SEQ ID NO:79) and R11723_PEA_{—}1_T6 (SEQ ID NO:80). Table 24 below describes the starting and ending position of this segment on each transcript.

TABLE 24
Segment location on transcripts

| | | Segment |
|---|---|---|
| | Segment | ending |
| Transcript name | starting position | position |
| R11723_PEA_1_T15 (SEQ ID NO: 75) | 624 | 681 |
| R11723_PEA_1_T5 (SEQ ID NO: 79) | 777 | 834 |
| R11723_PEA_1_T6 (SEQ ID NO: 80) | 811 | 868 |

Segment cluster R11723_PEA_{—}1_node_{—}20 (SEQ ID NO:460) according to the present invention can be found in the following transcript(s): R11723_PEA_{—}1_T5 (SEQ ID NO:79) and R11723_PEA_{—}1_T6 (SEQ ID NO:80). Table 25 below describes the starting and ending position of this segment on each transcript.

TABLE 25
Segment location on transcripts

| | | Segment |
|---|---|---|
| | Segment | ending |
| Transcript name | starting position | position |
| R11723_PEA_1_T5 (SEQ ID NO: 79) | 1009 | 1019 |
| R11723_PEA_1_T6 (SEQ ID NO: 80) | 1043 | 1053 |

Segment cluster R11723_PEA_{—}1_node_{—}21 (SEQ ID NO:461) according to the present invention is supported by 36 libraries. The number of libraries was determined as previously described. This segment can be found in the following transcript(s): R11723_PEA_{—}1_T5 (SEQ ID NO:79) and R11723_PEA_{—}1_T6 (SEQ ID NO:80). Table 26 below describes the starting and ending position of this segment on each transcript.

TABLE 26
Segment location on transcripts

| | | Segment |
|---|---|---|
| | Segment | ending |
| Transcript name | starting position | position |
| R11723_PEA_1_T5 (SEQ ID NO: 79) | 1020 | 1082 |
| R11723_PEA_1_T6 (SEQ ID NO: 80) | 1054 | 1116 |

Segment cluster R11723_PEA_{—}1_node_{—}23 (SEQ ID NO:462) according to the present invention is supported by 39 libraries. The number of libraries was determined as previously described. This segment can be found in the following transcript(s): R11723_PEA_{—}1_T5 (SEQ ID NO:79) and R11723_PEA_{—}1_T6 (SEQ ID NO:80). Table 27 below describes the starting and ending position of this segment on each transcript.

TABLE 27
Segment location on transcripts

| | | Segment |
|---|---|---|
| | Segment | ending |
| Transcript name | starting position | position |
| R11723_PEA_1_T5 (SEQ ID NO: 79) | 1570 | 1599 |
| R11723_PEA_1_T6 (SEQ ID NO: 80) | 1604 | 1633 |

Segment cluster R11723_PEA_{—}1_node_{—}24 (SEQ ID NO:463) according to the present invention is supported by 51 libraries. The number of libraries was determined as previously described. This segment can be found in the following transcript(s): R11723_PEA_{—}1_T15 (SEQ ID NO:75), R11723_PEA_{—}1_T5 (SEQ ID NO:79) and R11723_PEA_{—}1_T6 (SEQ ID NO:80). Table 28 below describes the starting and ending position of this segment on each transcript.

TABLE 28
Segment location on transcripts

| | | Segment |
|---|---|---|
| | Segment | ending |
| Transcript name | starting position | position |
| R11723_PEA_1_T15 (SEQ ID NO: 75) | 682 | 765 |
| R11723_PEA_1_T5 (SEQ ID NO: 79) | 1600 | 1683 |
| R11723_PEA_1_T6 (SEQ ID NO: 80) | 1634 | 1717 |

Segment cluster R11723_PEA_{—}1_node_{—}25 (SEQ ID NO:464) according to the present invention is supported by 54 libraries. The number of libraries was determined as previously described. This segment can be found in the following transcript(s): R11723_PEA_{—}1_T15 (SEQ ID NO:75), R11723_PEA_{—}1_T5 (SEQ ID NO:79) and R11723_PEA_{—}1_T6 (SEQ ID NO:80). Table 29 below describes the starting and ending position of this segment on each transcript.

TABLE 29
Segment location on transcripts

| | | Segment |
|---|---|---|
| | Segment | ending |
| Transcript name | starting position | position |
| R11723_PEA_1_T15 (SEQ ID NO: 75) | 766 | 791 |
| R11723_PEA_1_T5 (SEQ ID NO: 79) | 1684 | 1709 |
| R11723_PEA_1_T6 (SEQ ID NO: 80) | 1718 | 1743 |

Segment cluster R11723_PEA_{—}1_node_{—}26 (SEQ ID NO:465) according to the present invention is supported by 62 libraries. The number of libraries was determined as previously described. This segment can be found in the following transcript(s): R11723_PEA_{—}1_T15 (SEQ ID NO:75), R11723_PEA_{—}1_T5 (SEQ ID NO:79) and R11723_PEA_{—}1_T6 (SEQ ID NO:80). Table 30 below describes the starting and ending position of this segment on each transcript.

TABLE 30
Segment location on transcripts

| | | Segment |
|---|---|---|
| | Segment | ending |
| Transcript name | starting position | position |
| R11723_PEA_1_T15 (SEQ ID NO: 75) | 792 | 904 |
| R11723_PEA_1_T5 (SEQ ID NO: 79) | 1710 | 1822 |
| R11723_PEA_1_T6 (SEQ ID NO: 80) | 1744 | 1856 |

Segment cluster R11723_PEA_{—}1_node_{—}27 (SEQ ID NO:466) according to the present invention is supported by 67 libraries. The number of libraries was determined as previously described. This segment can be found in the following transcript(s): R11723=PEA_{—}1_T15 (SEQ ID NO:75), R11723_PEA_{—}1_T5 (SEQ ID NO:79) and R11723_PEA_{—}1_T6 (SEQ ID NO:80). Table 31 below describes the starting and ending position of this segment on each transcript.

TABLE 31
Segment location on transcripts

| | | Segment |
|---|---|---|
| | Segment | ending |
| Transcript name | starting position | position |
| R11723_PEA_1_T15 (SEQ ID NO: 75) | 905 | 986 |
| R11723_PEA_1_T5 (SEQ ID NO: 79) | 1823 | 1904 |
| R11723_PEA_1_T6 (SEQ ID NO: 80) | 1857 | 1938 |

Segment cluster R11723_PEA_{—}1_node_{—}28 (SEQ ID NO:467) according to the present invention can be found in the following transcript(s): R11723_PEA_{—}1_T15 (SEQ ID NO:75), R11723_PEA_{—}1_T5 (SEQ ID NO:79) and R11723_PEA_{—}1_T6 (SEQ ID NO:80). Table 32 below describes the starting and ending position of this segment on each transcript.

TABLE 32
Segment location on transcripts

| | | Segment |
|---|---|---|
| | Segment | ending |
| Transcript name | starting position | position |
| R11723_PEA_1_T15 (SEQ ID NO: 75) | 987 | 1010 |
| R11723_PEA_1_T5 (SEQ ID NO: 79) | 1905 | 1928 |
| R11723_PEA_1_T6 (SEQ ID NO: 80) | 1939 | 1962 |

Segment cluster R11723_PEA_{—}1_node_{—}29 (SEQ ID NO:468) according to the present invention is supported by 69 libraries. The number of libraries was determined as previously described. This segment can be found in the following transcript(s): R11723_PEA_{—}1_T15 (SEQ ID NO:75), R11723_PEA_{—}1_T5 (SEQ ID NO:79) and R11723_PEA_{—}1_T6 (SEQ ID NO:80). Table 33 below describes the starting and ending position of this segment on each transcript.

TABLE 33
Segment location on transcripts

| | | Segment |
|---|---|---|
| | Segment | ending |
| Transcript name | starting position | position |
| R11723_PEA_1_T15 (SEQ ID NO: 75) | 1011 | 1038 |
| R11723_PEA_1_T5 (SEQ ID NO: 79) | 1929 | 1956 |
| R11723_PEA_1_T6 (SEQ ID NO: 80) | 1963 | 1990 |

Segment cluster R11723_PEA_{—}1_node_{—}3 (SEQ ID NO:469) according to the present invention can be found in the following transcript(s): R11723_PEA_{—}1_T15 (SEQ ID NO:75), R11723_PEA_{—}1_T17 (SEQ ID NO:76), R11723_PEA_{—}1_T19 (SEQ ID NO:77), R11723_PEA_{—}1_T20 (SEQ ID NO:78), R11723_PEA_{—}1_T5 (SEQ ID NO:79) and R11723_PEA_{—}1_T6 (SEQ ID NO:80). Table 34 below describes the starting and ending position of this segment on each transcript.

TABLE 34
Segment location on transcripts

| | | Segment |
|---|---|---|
| | Segment | ending |
| Transcript name | starting position | position |
| R11723_PEA_1_T15 (SEQ ID NO: 75) | 310 | 319 |
| R11723_PEA_1_T17 (SEQ ID NO: 76) | 310 | 319 |
| R11723_PEA_1_T19 (SEQ ID NO: 77) | 310 | 319 |
| R11723_PEA_1_T20 (SEQ ID NO: 78) | 310 | 319 |
| R11723_PEA_1_T5 (SEQ ID NO: 79) | 310 | 319 |
| R11723_PEA_1_T6 (SEQ ID NO: 80) | 310 | 319 |

Segment cluster R11723_PEA_{—}1_node_{—}30 (SEQ ID NO:470) according to the present invention can be found in the following transcript(s): R11723_PEA_{—}1_T15 (SEQ ID NO:75), R11723_PEA_{—}1_T5 (SEQ ID NO:79) and R11723_PEA_{—}1_T6 (SEQ ID NO:80). Table 35 below describes the starting and ending position of this segment on each transcript.

TABLE 35
Segment location on transcripts

| | Segment | |
|---|---|---|
| | starting | Segment ending |
| Transcript name | position | position |
| R11723_PEA_1_T15 (SEQ ID NO: 75) | 1039 | 1059 |
| R11723_PEA_1_T5 (SEQ ID NO: 79) | 1957 | 1977 |
| R11723_PEA_1_T6 (SEQ ID NO: 80) | 1991 | 2011 |

Segment cluster R11723_PEA_{—}1_node_{—}4 (SEQ ID NO:471) according to the present invention is supported by 25 libraries. The number of libraries was determined as previously described. This segment can be found in the following transcript(s): R11723_PEA_{—}1_T15 (SEQ ID NO:75), R11723_PEA_{—}1_T17 (SEQ ID NO:76), R11723_PEA_{—}1_T19 (SEQ ID NO:77), R11723_PEA_{—}1_T20 (SEQ ID NO:78), R11723_PEA_{—}1_T5 (SEQ ID NO:79) and R11723_PEA_{—}1_T6 (SEQ ID NO:80). Table 36 below describes the starting and ending position of this segment on each transcript.

TABLE 36
Segment location on transcripts

| | Segment | |
|---|---|---|
| | starting | Segment ending |
| Transcript name | position | position |
| R11723_PEA_1_T15 (SEQ ID NO: 75) | 320 | 371 |
| R11723_PEA_1_T17 (SEQ ID NO: 76) | 320 | 371 |
| R11723_PEA_1_T19 (SEQ ID NO: 77) | 320 | 371 |
| R11723_PEA_1_T20 (SEQ ID NO: 78) | 320 | 371 |
| R11723_PEA_1_T5 (SEQ ID NO: 79) | 320 | 371 |
| R11723_PEA_1_T6 (SEQ ID NO: 80) | 320 | 371 |

Segment cluster R11723_PEA_{—}1_node_{—}5 (SEQ ID NO:472) according to the present invention is supported by 26 libraries. The number of libraries was determined as previously described. This segment can be found in the following transcript(s): R11723_PEA_{—}1_T15 (SEQ ID NO:75), R11723_PEA_{—}1_T17 (SEQ ID NO:76), R11723_PEA_{—}1_T19 (SEQ ID NO:77), R11723_PEA_{—}1_T20 (SEQ ID NO:78), R11723_PEA_{—}1_T5 (SEQ ID NO:79) and R11723_PEA_{—}1_T6 (SEQ ID NO:80). Table 37 below describes the starting and ending position of this segment on each transcript.

TABLE 37
Segment location on transcripts

| | Segment | |
|---|---|---|
| | starting | Segment ending |
| Transcript name | position | position |
| R11723_PEA_1_T15 (SEQ ID NO: 75) | 372 | 414 |
| R11723_PEA_1_T17 (SEQ ID NO: 76) | 372 | 414 |
| R11723_PEA_1_T19 (SEQ ID NO: 77) | 372 | 414 |
| R11723_PEA_1_T20 (SEQ ID NO: 78) | 372 | 414 |
| R11723_PEA_1_T5 (SEQ ID NO: 79) | 372 | 414 |
| R11723_PEA_1_T6 (SEQ ID NO: 80) | 372 | 414 |

Segment cluster R11723_PEA_{—}1_node_{—}6 (SEQ ID NO:473) according to the present invention is supported by 27 libraries. The number of libraries was determined as previously described. This segment can be found in the following transcript(s): R11723_PEA_{—}1_T15 (SEQ ID NO:75), R11723_PEA_{—}1_T17 (SEQ ID NO:76), R11723_PEA_{—}1_T19 (SEQ ID NO:77), R11723_PEA_{—}1_T20 (SEQ ID NO:78), R11723_PEA_{—}1_T5 (SEQ ID NO:79) and R11723_PEA_{—}1_T6 (SEQ ID NO:80). Table 38 below describes the starting and ending position of this segment on each transcript.

TABLE 38
Segment location on transcripts

| | Segment | |
|---|---|---|
| | starting | Segment ending |
| Transcript name | position | position |
| R11723_PEA_1_T15 (SEQ ID NO: 75) | 415 | 446 |
| R11723_PEA_1_T17 (SEQ ID NO: 76) | 415 | 446 |
| R11723_PEA_1_T19 (SEQ ID NO: 77) | 415 | 446 |
| R11723_PEA_1_T20 (SEQ ID NO: 78) | 415 | 446 |
| R11723_PEA_1_T5 (SEQ ID NO: 79) | 415 | 446 |
| R11723_PEA_1_T6 (SEQ ID NO: 80) | 415 | 446 |

Segment cluster R11723_PEA_{—}1_node_{—}7 (SEQ ID NO:474) according to the present invention is supported by 29 libraries. The number of libraries was determined as previously described. This segment can be found in the following transcript(s): R11723_PEA_{—}1_T15 (SEQ ID NO:75), R11723_PEA_{—}1_T17 (SEQ ID NO:76), R11723_PEA_{—}1_T19 (SEQ ID NO:77), R11723_PEA_{—}1_T20 (SEQ ID NO:78), R11723_PEA_{—}1_T5 (SEQ ID NO:79) and R11723_PEA_{—}1_T6 (SEQ ID NO:80). Table 39 below describes the starting and ending position of this segment on each transcript.

TABLE 39
Segment location on transcripts

| | Segment | |
|---|---|---|
| | starting | Segment ending |
| Transcript name | position | position |
| R11723_PEA_1_T15 (SEQ ID NO: 75) | 447 | 485 |
| R11723_PEA_1_T17 (SEQ ID NO: 76) | 447 | 485 |
| R11723_PEA_1_T19 (SEQ ID NO: 77) | 447 | 485 |
| R11723_PEA_1_T20 (SEQ ID NO: 78) | 447 | 485 |
| R11723_PEA_1_T5 (SEQ ID NO: 79) | 447 | 485 |
| R11723_PEA_1_T6 (SEQ ID NO: 80) | 447 | 485 |

Segment cluster R11723_PEA_{—}1_node_{—}8 (SEQ ID NO:475) according to the present invention is supported by 2 libraries. The number of libraries was determined as previously described. This segment can be found in the following transcript(s): R11723_PEA_{—}1_T6 (SEQ ID NO:80). Table 40 below describes the starting and ending position of this segment on each transcript.

TABLE 40
Segment location on transcripts

| | Segment | |
|---|---|---|
| | starting | Segment ending |
| Transcript name | position | position |
| R11723_PEA_1_T6 (SEQ ID NO: 80) | 486 | 519 |

It should be noted that the variants of this cluster are variants of the hypothetical protein PSEC0181 (SEQ ID NO:1395) (referred to herein as “PSEC”). Furthermore, use of the known protein (WT protein) for detection of ovarian cancer, alone or in combination with one or more variants of this cluster and/or of any other cluster and/or of any known marker, also comprises an embodiment of the present invention.

It should be noted that the nucleotide transcript sequence of known protein (PSEC, also referred to herein as the “wild type” or WT protein) feature at least one SNP that appears to affect the coding region, in addition to certain silent SNPs. This SNP does not have an effect on the R11723_PEA_{—}1_T5 (SEQ ID NO:79) splice variant sequence): “G->” resulting in a missing nucleotide (affects amino acids from position 91 onwards). The missing nucleotide creates a frame shift, resulting in a new protein. This SNP was not previously identified and is supported by 5 ESTs out of ˜70 ESTs in this exon.

Expression of R1723 Transcripts, which are Detectable by Amplicon as Depicted in Sequence Name R11723 Seg13 (SEQ ID NO:1297) in Normal and Cancerous Colon Tissues.

Expression of transcripts detectable by or according to seg13, R11723 seg13 amplicon (SEQ ID NO: 1297) and R11723 seg13F (SEQ ID NO: 1295) and R11723 seg13R (SEQ ID NO: 1296) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); HPRT1-amplicon, SEQ ID NO:615), and RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 3, above: “Tissue samples in colon cancer testing panel”), to obtain a value of fold differential expression for each sample relative to median of the normal PM samples.

FIG. 28 is a histogram showing differential expression of the above-indicated transcripts in cancerous colon samples relative to the normal samples. Values represent the average of duplicate experiments. Error bars indicate the minimal and maximal values obtained.

As is evident from FIG. 28, the expression of transcripts detectable by the above amplicon in a few cancer samples was higher by more than 5 fold than in the non-cancerous samples (Sample Nos. 41, 52, 62-67, 69-71 Table 3: “Tissue samples in colon cancer testing panel”). However, the expression of transcripts detectable by the above amplicon in a several other cancer samples was lower than in the non-cancerous samples.

Primer pairs are also optionally and preferably encompassed within the present invention; for example, for the above experiment, the following primer pair was used as a non-limiting illustrative example only of a suitable primer pair: R11723 seg13F forward primer (SEQ ID NO: 1295); and R11723 seg13R reverse primer (SEQ ID NO: 1296).

The present invention also preferably encompasses any amplicon obtained through the use of any suitable primer pair; for example, for the above experiment, the following amplicon was obtained as a non-limiting illustrative example only of a suitable amplicon: R11723 seg13 (SEQ ID NO: 1297).

(SEQ ID NO: 1295)

R11723seg13F-ACACTAAAAGAACAAACACCTTGCTC

(SEQ ID NO: 1296)

R11723seg13R-TCCTCAGAAGGCACATGAAAGA
R11723seg13-amplicon (SEQ ID NO: 1297):
ACACTAAAAGAACAAACACCTTGCTCTTCGAGATGAGACATTTTGCCAAG
CAGTTGACCACTTAGTTCTCAAGAAGCAAGTATCTCTTTCATGTGCCTTC
TGAGGA

Expression of R11723 Transcripts, which are Detectable by Amplicon as Depicted in Sequence Name R11723 junc11-18 (SEQ ID NO: 1300) in Normal and Cancerous Colon Tissues.

Expression of transcripts detectable by or according to junc11-18, R11723 junc11-18 amplicon (SEQ ID NO: 1300) and R11723 junc11-18F (SEQ ID NO: 1298) and R11723 junc11-18R (SEQ ID NO: 1299) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); HPRT1-amplicon, SEQ ID NO:615), and RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 3, above: “Tissue samples in colon cancer testing panel”), to obtain a value of fold differential expression for each sample relative to median of the normal PM samples.

FIG. 29 is a histogram showing differential expression of the above-indicated transcripts in a few cancerous colon samples relative to the normal samples (Sample Nos. 41, 52, 62-67, 69-71 Table 3: “Tissue samples in colon cancer testing panel”).

As is evident from FIG. 29, the expression of transcripts detectable by the above amplicon in a few cancer samples was higher by more than 5 fold than in the non-cancerous samples (Sample Nos. 41, 52, 62-67, 69-71 Table 1: “Tissue samples in colon cancer testing panel”). However, the expression of transcripts detectable by the above amplicon in a several other cancer samples was lower than in the non-cancerous samples Primer pairs are also optionally and preferably encompassed within the present invention; for example, for the above experiment, the following primer pair was used as a non-limiting illustrative example only of a suitable primer pair: R11723 junc11-18F forward primer (SEQ ID NO: 1298); and R11723 junc11-18R reverse primer (SEQ ID NO: 1299).

The present invention also preferably encompasses any amplicon obtained through the use of any suitable primer pair; for example, for the above experiment, the following amplicon was obtained as a non-limiting illustrative example only of a suitable amplicon: R11723 junc11-18 (SEQ ID NO: 1300).

(SEQ ID NO: 1298)

R11723junc11-18F-AGTGATGGAGCAAAGTGCCG

(SEQ ID NO: 1299)

R11723 junc11-18R-CAGCAGCTGATGCAAACTGAG

(SEQ ID NO: 1300)

R11723 junc11-18 amplicon
AGTGATGGAGCAAAGTGCCGGGATCATGTACCGCAAGTCCTGTGCATCAT
CAGCGGCCTGTCTCATCGCCTCTGCCGGGTACCAGTCCTTCTGCTCCCCA
GGGAAACTGAACTCAGTTTGCATCAGCTGCTG

Expression of R11723 Transcripts, which are Detectable by Amplicon as Depicted in Sequence Name R11723Seg13 (SEQ ID NO: 1297) in different normal tissues.

Expression of R11723 transcripts detectable by or according to R11723seg13 amplicon (SEQ ID NO: 1297) and R11723seg13F (SEQ ID NO: 1295), R11723seg13R (SEQ ID NO: 1296) was measured by real time PCR. In parallel the expression of four housekeeping genes—RPL19 (GenBank Accession No. NM_{—}000981 (SEQ ID NO:1580); RPL19 amplicon, SEQ ID NO:1264), TATA box (GenBank Accession No. NM_{—}003194 (SEQ ID NO:1581); TATA amplicon, SEQ ID NO:1267), UBC (GenBank Accession No. BC000449 (SEQ ID NO:1582); amplicon—Ubiquitin-amplicon, SEQ ID NO:1270) and SDHA (GenBank Accession No. NM_{—}004168 (SEQ ID NO:1583); amplicon—SDHA-amplicon, SEQ ID NO:1273) was measured similarly. For each RT sample, the expression of the above amplicon was normalized to the geometric mean of the quantities of the housekeeping genes. The normalized quantity of each RT sample was then divided by the median of the quantities of the ovary samples to obtain a value of relative expression of each sample relative to median of the ovary samples.

The results are described in FIG. 30, presenting the histogram showing the expression of R11723 transcripts, detectable by amplicon depicted in sequence name R11723seg13 (SEQ ID NO: 1297) in different normal tissues.

(SEQ ID NO: 1295)

R11723seg13F-ACACTAAAAGAACAAACACCTTGCTC

(SEQ ID NO: 1296)

R11723seg13R-TCCTCAGAAGGCACATGAAAGA
R11723seg13-amplicon (SEQ ID NO: 1297):
ACACTAAAAGAACAAACACCTTGCTCTTCGAGATGAGACATTTTGCCAAG
CAGTTGACCACTTAGTTCTCAAGAAGCAACTATCTCTTTCATGTGCCTTC
TGAGGA

Expression of R11723 Transcripts, which are Detectable by Amplicon as Depicted in Sequence Name R11723 junc11-18 (SEQ ID NO: 1300) in Different Normal Tissues.

Expression of R11723 transcripts detectable by or according to R11723seg13 amplicon (SEQ ID NO: 1300) and R11723 junc11-18F (SEQ ID NO:1298), R11723 junc11-18R (SEQ ID NO:1299) was measured by real time PCR. In parallel the expression of four housekeeping genes—RPL19 (GenBank Accession No. NM_{—}000981 (SEQ ID NO:1580); RPL19 amplicon, SEQ ID NO:1264), TATA box (GenBank Accession No. NM_{—}003194 (SEQ ID NO:1581); TATA amplicon, SEQ ID NO:1267), UBC (GenBank Accession No. BC000449 (SEQ ID NO:1582); amplicon—Ubiquitin-amplicon, SEQ ID NO:1270) and SDHA (GenBank Accession No. NM_{—}004168 (SEQ ID NO:1583); amplicon—SDHA-amplicon, SEQ ID NO:1273) was measured similarly. For each RT sample, the expression of the above amplicon was normalized to the geometric mean of the quantities of the housekeeping genes. The normalized quantity of each RT sample was then divided by the median of the quantities of the ovary samples to obtain a value of relative expression of each sample relative to median of the ovary samples.

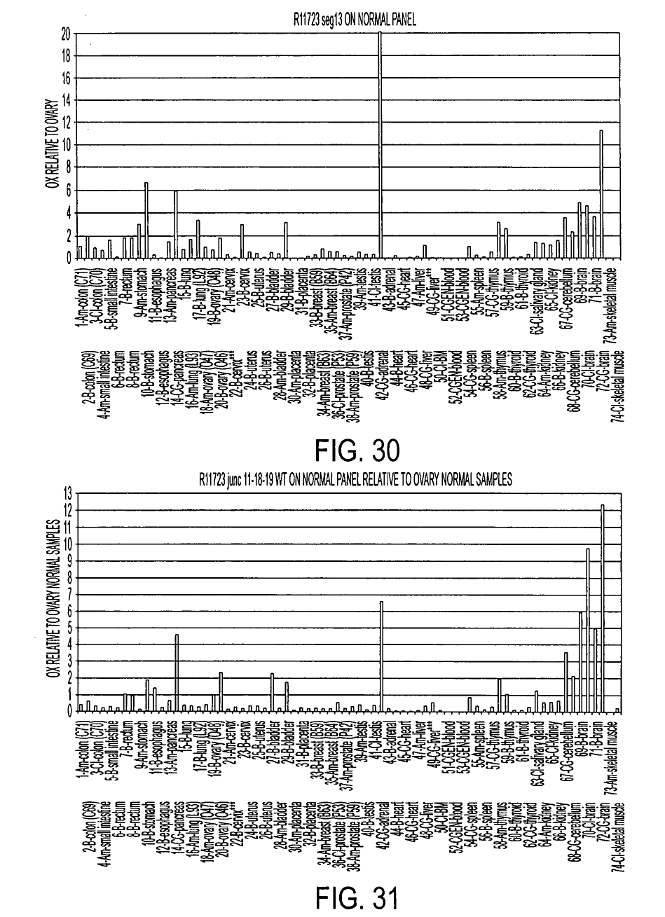

The results are described in FIG. 31, presenting the histogram showing the expression of R11723 transcripts, detectable by amplicon depicted in sequence name R11723 junc11-18 (SEQ ID NO: 1300) in different normal tissues.

(SEQ ID NO: 1298)

R11723junc11-18F-AGTGATGGAGCAAAGTGCCG

(SEQ ID NO: 1299)

R11723junc11-18R-CAGCAGCTGATGCAAACTGAG

(SEQ ID NO: 1300)

R11723junc11-18 amplicon-AGTGATGGAGCAAAGTGCCGGGATC
ATGTACCGCAAGTCCTGTGCATCATCAGCGGCCTGTCTCATCGCCTCTGC
CGGGTACCAGTCCTTCTGCTCCCCAGGGAAACTGAACTCAGTTTGCATCA
GCTGCTG

It was found that the known protein (wild type) transcript expression pattern for the above cluster (PSEC) is similar to the variant expression pattern, except that in some cases (such as ovarian cancer) the variant overexpression in cancer was found to be higher.

### Variant Protein Alignment to the Previously Known Protein:

Sequence name: /tmp/gp6eQTLWqk/mFtjUpUzhb:Q8IXMO
Sequence documentation:
Alignment of: R11723_PEA_1_P6 (SEQ ID NO:604) × Q8IXMO (SEQ ID NO:1393) ..
Alignment segment 1/1:

| | | | |
|---|---|---|---|
| Quality: | 1128.00 | Escore: | 0 |
| Matching length: | 112 | Total length: | 112 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

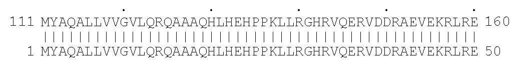

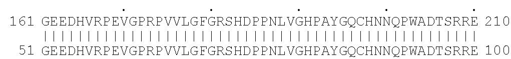

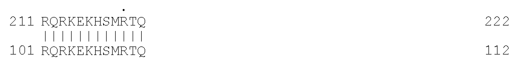

Sequence name: /tmp/gp6eQTLWqk/mFtjUpUzhb:Q9GAC2
Sequence documentation:
Alignment of: R11723_PEA_1_P6 (SEQ ID NO:604) × Q96AC2 (SEQ ID NO:1394) ..
Alignment segment 1/1:

| | | | |
|---|---|---|---|
| Quality: | 835.00 | Escore: | 0 |
| Matching length: | 83 | Total length: | 83 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

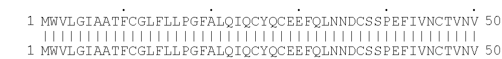

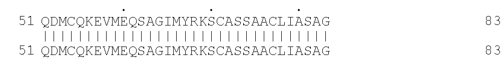

Sequence name: /tmp/gp6eQTLWqk/mFtjUpUzhb:Q8N2G4
Sequence documentation:
Alignment of: R11723_PEA_1_PG (SEQ ID NO:604) × Q8N2G4 (SEQ ID NO:1395)
Alignment segment 1/1:

| | | | |
|---|---|---|---|
| Quality: | 835.00 | Escore: | 0 |
| Matching length: | 83 | Total length: | 83 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

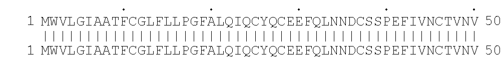

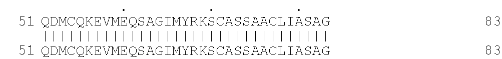

Sequence name: /tmp/gp6eQTLWqk/mFtjUpUzhb:BAC85518 (SEQ ID NO: 1396)
Sequence documentation:
Alignment of: R11723_PEA_1_P6 (SEQ ID NO:604) × BAC85518 (SEQ ID NO:1396)
Alignment segment 1/1:

| | | | |
|---|---|---|---|
| Quality: | 835.00 | Escore: | 0 |
| Matching length: | 83 | Total length: | 83 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

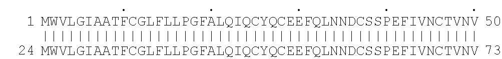

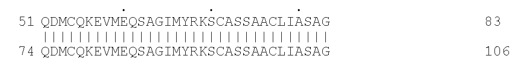

Sequence name: /tmp/VXjdFlzdBX/bexTxTh0Th:Q96AC2 (SEQ ID NO:1394)
Sequence documentation:
Alignment of: R11723_PEA_1_P7 (SEQ ID NO:605) × Q96AC2 (SEQ ID NO:1394)
Alignment segment 1/1:

| | | | |
|---|---|---|---|
| Quality: | 654.00 | Escore: | 0 |
| Matching length: | 64 | Total length: | 64 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

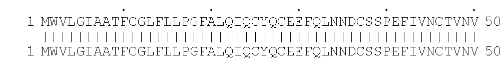

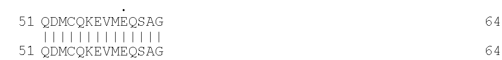

Sequence name: /tmp/VXjdFlzdBX/bexTxTh0Th:Q8N2G4 (SEQ ID NO:1395)
Sequence documentation:
Alignment of: R11723_PEA_1_P7 (SEQ ID NO:605) × Q8N2G4 (SEQ ID NO:1395) ..
Alignment segment 1/1:

| | | | |
|---|---|---|---|
| Quality: | 654.00 | Escore: | 0 |
| Matching length: | 64 | Total length: | 64 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

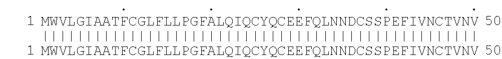

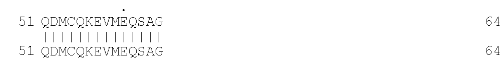

Sequence name: /tmp/VXjdFlzdBX/bexTxTh0Th:BAC85273 (SEQ ID NO:1397)
Sequence documentation:
Alignment of: R11723_PEA_1_P7 (SEQ ID NO:605) × BAC85273 (SEQ ID MO:1397)
Alignment segment 1/1:

| | | | |
|---|---|---|---|
| Quality: | 600.00 | Escore: | 0 |
| Matching length: | 59 | Total length: | 59 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

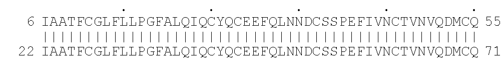

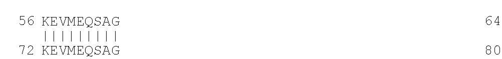

Sequence name: /tmp/VXjdF1zdBX/bexTxTh0Th:BAC85518 (SEQ ID NO: 1396)
Sequence documentation:
Alignment of: R11723_PEA_1_P7 (SEQ ID NO:605) × BAC85518 (SEQ ID NO:1396)
Alignment segment 1/1:

| | | | |
|---|---|---|---|
| Quality: | 654.00 | Escore: | 0 |
| Matching length: | 64 | Total length: | 64 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

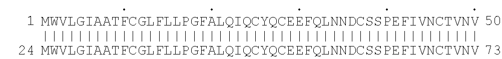

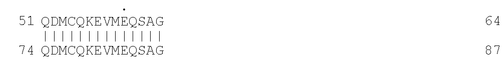

Sequence name: /tmp/OLMSexEmIh/pc7Z7Xm1YR:Q96AC2 (SEQ ID NO: 1394)
Sequence documentation:
Alignment of: R11723_PEA_1_P10 (SEQ ID NO:607) × Q96AC2 (SEQ ID NO:1394) ..
Alignment segment 1/1:

| | | | |
|---|---|---|---|
| Quality: | 645.00 | Escore: | 0 |
| Matching length: | 63 | Total length: | 63 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

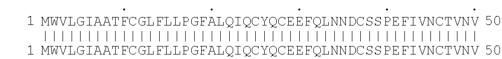

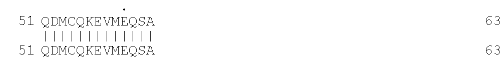

Sequence name: /tmp/OLMSexEmIh/pc7Z7Xm1YR:Q8N2G4 (SEQ ID NO:1395)
Sequence documentation:
Alignment of: R11723_PEA_1_P10 (SEQ ID NO:607) × Q8N2G4 (SEQ ID NO:1395) ..
Alignment segment 1/1:

| | | | |
|---|---|---|---|
| Quality: | 645.00 | Escore: | 0 |
| Matching length: | 63 | Total length: | 63 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

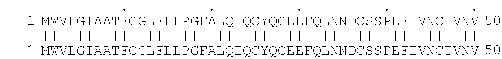

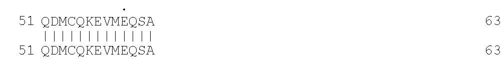

Sequence name: /tmp/OLMSexEmIh/pc7Z7Xm1YR:BAC85273 (SEQ ID NO:1397)
Sequence documentation:
Alignment of: R11723_PEA_1_P10 (SEQ ID NO:607) × BAC85273 (SEQ ID NO:1397) ..
Alignment segment 1/1:

| | | | |
|---|---|---|---|
| Quality: | 591.00 | Escore: | 0 |
| Matching length: | 58 | Total length: | 58 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

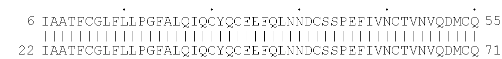

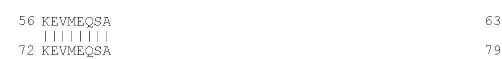

Sequence name: /tmp/OLMSexEmIh/pc7Z7Xm1YR:BAC85518 (SEQ ID NO:1396)
Sequence documentation:
Alignment of: R11723_PEA_1_P10 (SEQ ID NO:607) × BAC85518 (SEQ ID NO:1396) ..
Alignment segment 1/1:

| | | | |
|---|---|---|---|
| Quality: | 645.00 | Escore: | 0 |
| Matching length: | 63 | Total length: | 63 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

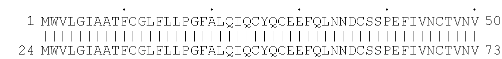

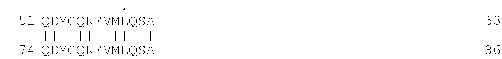

Alignment of: R11723_PEA_1_P13 (SEQ ID NO:606) × Q96AC2 (SEQ ID NO:1394) ..
Alignment segment 1/1:

| | | | |
|---|---|---|---|
| Quality: | 645.00 | Escore: | 0 |
| Matching length: | 63 | Total length: | 63 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

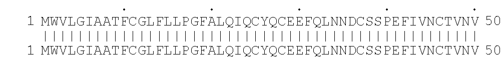

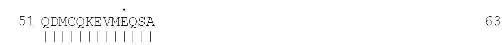

### Description for Cluster M77903

Cluster M77903 features 4 transcript(s) and 29 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| M77903_T11 | 81 |
| M77903_T12 | 82 |
| M77903_T34 | 83 |
| M77903_T36 | 84 |

TABLE 2
Segments of interest

| Segment Name | SEQ ID NO: |
|---|---|
| M77903_node_2 | 476 |
| M77903_node_13 | 477 |
| M77903_node_16 | 478 |
| M77903_node_18 | 479 |
| M77903_node_35 | 480 |
| M77903_node_36 | 481 |
| M77903_node_37 | 482 |
| M77903_node_38 | 483 |
| M77903_node_40 | 484 |
| M77903_node_44 | 485 |
| M77903_node_46 | 486 |
| M77903_node_47 | 487 |
| M77903_node_48 | 488 |
| M77903_node_49 | 489 |
| M77903_node_51 | 490 |
| M77903_node_52 | 491 |
| M77903_node_1 | 492 |
| M77903_node_5 | 493 |
| M77903_node_9 | 494 |
| M77903_node_10 | 495 |
| M77903_node_11 | 496 |
| M77903_node_12 | 497 |
| M77903_node_15 | 498 |
| M77903_node_17 | 499 |
| M77903_node_20 | 500 |
| M77903_node_28 | 501 |
| M77903_node_34 | 502 |
| M77903_node_41 | 503 |
| M77903_node_42 | 504 |

TABLE 3
Proteins of interest

| | SEQ ID | |
|---|---|---|
| Protein Name | NO: | Corresponding Transcript(s) |
| M77903_P4 | 608 | M77903_T11 (SEQ ID NO: 81) |
| M77903_P5 | 609 | M77903_T12 (SEQ ID NO: 82) |
| M77903_P15 | 610 | M77903_T34 (SEQ ID NO: 83) |
| M77903_P16 | 611 | M77903_T36 (SEQ ID NO: 84) |

These sequences are variants of the known protein Translocon-associated protein, alpha subunit precursor (SwissProt accession identifier SSRA_HUMAN; known also according to the synonyms TRAP-alpha; Signal sequence receptor alpha subunit; SSR-alpha), SEQ ID NO: 641, referred to herein as the previously known protein.

Protein Translocon-associated protein (SEQ ID NO:641), alpha subunit precursor is known or believed to have the following function(s): TRAP proteins are part of a complex whose function is to bind calcium to the ER membrane and thereby regulate the retention of ER resident proteins. May be involved in the recycling of the translocation apparatus after completion of the translocation process or may function as a membrane-bound chaperone facilitating folding of translocated proteins. The sequence for protein Translocon-associated protein, alpha subunit precursor is given at the end of the application, as “Translocon-associated protein, alpha subunit precursor amino acid sequence”. Known polymorphisms for this sequence are as shown in Table 4.

TABLE 4
Amino acid mutations for Known Protein

| SNP position(s) on | |
|---|---|
| amino acid sequence | Comment |
| 28 | L -> S |
| 130 | Y -> H |

Protein Translocon-associated protein (SEQ ID NO:641), alpha subunit precursor localization is believed to be Type I membrane protein. Endoplasmic reticulum.

The following GO Annotation(s) apply to the previously known protein. The following annotation(s) were found: co-translational membrane targeting; positive control of cell proliferation, which are annotation(s) related to Biological Process; signal sequence receptor; calcium binding, which are annotation(s) related to Molecular Function; and endoplasmic reticulum; integral membrane protein, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster M77903 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 33 and Table 5. This cluster is overexpressed (at least at a minimum level) in the following pathological conditions: ovarian carcinoma and uterine malignancies.

TABLE 5
Normal tissue distribution

| Name of Tissue | Number |
|---|---|
| Adrenal | 120 |
| Bladder | 123 |
| Bone | 129 |
| Colon | 31 |
| Epithelial | 124 |
| General | 129 |
| head and neck | 263 |
| Kidney | 118 |
| Liver | 107 |
| Lung | 147 |
| Lymph nodes | 126 |
| Breast | 211 |
| bone marrow | 251 |
| Muscle | 109 |
| Ovary | 3 |
| Pancreas | 144 |
| Prostate | 142 |
| Skin | 163 |
| Stomach | 183 |
| T cells | 278 |
| Thyroid | 128 |
| Uterus | 81 |

TABLE 6
P values and ratios for expression in cancerous tissue

| Name of Tissue | P1 | P2 | SP1 | R3 | SP2 | R4 |
|---|---|---|---|---|---|---|
| adrenal | 3.8e−01 | 2.8e−01 | 4.1e−01 | 1.4 | 2.4e−01 | 1.6 |
| bladder | 3.7e−01 | 4.1e−01 | 1.6e−01 | 1.8 | 3.0e−01 | 1.4 |
| Bone | 2.0e−01 | 2.4e−01 | 7.7e−01 | 1.0 | 6.6e−01 | 0.9 |
| Brain | 5.9e−01 | 5.5e−01 | 8.4e−01 | 0.7 | 7.9e−01 | 0.8 |
| Colon | 7.0e−02 | 1.1e−02 | 6.1e−02 | 2.9 | 2.5e−02 | 3.2 |
| epithelial | 4.2e−02 | 5.8e−02 | 1.3e−01 | 1.2 | 4.7e−01 | 1.0 |
| general | 4.0e−02 | 2.0e−02 | 6.1e−01 | 1.0 | 8.9e−01 | 0.9 |
| head and neck | 4.5e−01 | 4.6e−01 | 1 | 0.4 | 9.0e−01 | 0.5 |
| kidney | 6.5e−01 | 7.6e−01 | 2.8e−01 | 1.2 | 5.3e−01 | 0.9 |
| Liver | 5.3e−01 | 5.8e−01 | 1 | 0.4 | 9.1e−01 | 0.6 |
| Lung | 6.1e−01 | 7.3e−01 | 3.7e−01 | 1.2 | 7.0e−01 | 0.9 |
| Lymph nodes | 2.4e−01 | 5.8e−01 | 7.1e−01 | 0.9 | 8.7e−01 | 0.6 |
| breast | 8.0e−01 | 8.3e−01 | 9.9e−01 | 0.4 | 9.1e−01 | 0.5 |
| bone marrow | 7.5e−01 | 6.8e−01 | 1 | 0.1 | 9.5e−01 | 0.5 |
| muscle | 4.0e−01 | 2.6e−01 | 6.2e−01 | 1.5 | 8.3e−01 | 0.7 |
| Ovary | 7.8e−03 | 8.7e−03 | 1.0e−02 | 5.8 | 3.1e−02 | 4.4 |
| pancreas | 5.6e−01 | 6.6e−01 | 7.8e−01 | 0.6 | 8.6e−01 | 0.6 |
| prostate | 4.5e−01 | 4.3e−01 | 6.2e−01 | 0.9 | 4.3e−01 | 0.8 |
| Skin | 4.9e−01 | 5.3e−01 | 3.6e−01 | 1.4 | 9.3e−01 | 0.4 |
| stomach | 2.9e−01 | 5.5e−01 | 7.5e−01 | 0.6 | 9.4e−01 | 0.5 |
| T cells | 6.7e−01 | 5.0e−01 | 5.5e−01 | 1.5 | 5.7e−01 | 1.1 |
| Thyroid | 5.7e−01 | 5.7e−01 | 7.4e−01 | 1.1 | 7.4e−01 | 1.1 |
| uterus | 7.4e−03 | 2.5e−02 | 4.6e−01 | 1.1 | 6.0e−01 | 0.9 |

As noted above, cluster M77903 features 4 transcript(s), which were listed in Table 1 above. These transcript(s) encode for protein(s) which are variant(s) of protein Translocon-associated protein (SEQ ID NO:641), alpha subunit precursor. A description of each variant protein according to the present invention is now provided.

Variant protein M77903_P4 (SEQ ID NO:608) according to the present invention has an amino acid sequence as given at the end of the application; it is encoded by transcript(s) M77903_T11 (SEQ ID NO:81). An alignment is given to the known protein (Translocon-associated protein (SEQ ID NO:641), alpha subunit precursor) at the end of the application. One or more alignments to one or more previously published protein sequences are given at the end of the application. A brief description of the relationship of the variant protein according to the present invention to each such aligned protein is as follows:

Comparison Report Between M77903_P4 (SEQ ID NO:608) and SSRA_HUMAN (SEQ ID NO:641):

1. An isolated chimeric polypeptide encoding for M77903_P4 (SEQ ID NO:608), comprising a first amino acid sequence being at least 90% homologous to MRLLPRLLLLLLLVFPATVLFRGGPRGLLAVAQDLTEDEETVEDSIIEDEDDEAEVEEDEPTDLVEDKEEED VSGEPEASPSADTTILFVKGEDFPANNIVKFLVGFTNKGTEDFIVESLDASFRYPQDYQFYIQNFTALPLNTV VPPQRQATFEYSFIPAEPMGGRPFGLVINLNYKDLNGNVFQDAVFNQTVTVIEREDGLDGET corresponding to amino acids 1-207 of SSRA_HUMAN (SEQ ID NO:641), which also corresponds to amino acids 1-207 of M77903_P4 (SEQ ID NO:608), and a second amino acid sequence being at least 70%, optionally at least 80%, preferably at least 85%, more preferably at least 90% and most preferably at least 95% homologous to a polypeptide having the sequence VRDPYRK (SEQ ID NO:1565) corresponding to amino acids 208-214 of M77903_P4 (SEQ ID NO:608), wherein said first amino acid sequence and second amino acid sequence are contiguous and in a sequential order.

2. An isolated polypeptide encoding for a tail of M77903_P4 (SEQ ID NO:608), comprising a polypeptide being at least 70%, optionally at least about 80%, preferably at least about 85%, more preferably at least about 90% and most preferably at least about 95% homologous to the sequence VRDPYRK (SEQ ID NO:1565) in M77903_P4 (SEQ ID NO:608).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein M77903_P4 (SEQ ID NO:608) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein M77903_P4 (SEQ ID NO:608) sequence provides support for the deduced sequence of this variant protein according to the present invention).

TABLE 7
Amino acid mutations

| SNP position(s) on amino acid | Alternative | |
|---|---|---|
| sequence | amino acid(s) | Previously known SNP? |
| 6 | R -> G | No |
| 24 | G -> | No |
| 28 | L -> S | Yes |
| 48 | E -> G | No |
| 54 | A -> T | Yes |
| 58 | E -> K | No |
| 63 | D -> | No |
| 89 | F -> | No |
| 116 | I -> M | No |
| 130 | Y -> H | No |
| 130 | Y -> N | No |
| 178 | K -> | No |

The glycosylation sites of variant protein M77903_P4 (SEQ ID NO:608), as compared to the known protein Translocon-associated protein (SEQ ID NO:641), alpha subunit precursor, are described in Table 8 (given according to their position(s) on the amino acid sequence in the first column; the second column indicates whether the glycosylation site is present in the variant protein; and the last column indicates whether the position is different on the variant protein).

TABLE 8
Glycosylation site(s)

| Position(s) on | | |
|---|---|---|
| known amino acid | Present in | Position in |
| sequence | variant protein? | variant protein? |
| 191 | Yes | 191 |
| 136 | Yes | 136 |

Variant protein M77903_P4 (SEQ ID NO:608) is encoded by the following transcript(s): M77903_T11 (SEQ ID NO:81), for which the sequence(s) is/are given at the end of the application. The coding portion of transcript M77903_T11 (SEQ ID NO:81) is shown in bold; this coding portion starts at position 200 and ends at position 841. The transcript also has the following SNPs as listed in Table 9 (given according to their position on the nucleotide sequence, with the alternative nucleic acid listed; the last column indicates whether the SNP is known or not; the presence of known SNPs in variant protein M77903_P4 (SEQ ID NO:608) sequence provides support for the deduced sequence of this variant protein according to the present invention).

TABLE 9
Nucleic acid SNPs

| SNP position on nucleotide | Alternative | Previously |
|---|---|---|
| sequence | nucleic acid | knowns SNP? |
| 72 | G -> T | Yes |
| 120 | C -> G | No |
| 147 | G -> C | Yes |
| 215 | C -> G | No |
| 271 | C -> | No |
| 282 | T -> C | Yes |
| 342 | A -> G | No |
| 359 | G -> A | Yes |
| 371 | G -> A | No |
| 388 | T -> | No |
| 464 | T -> | No |
| 547 | T -> C | No |
| 547 | T -> G | No |
| 587 | T -> A | No |
| 587 | T -> C | No |
| 731 | A -> | No |
| 927 | A -> | No |
| 1117 | T -> | No |
| 1118 | G -> | No |
| 1296 | T -> A | Yes |
| 1324 | T -> | No |
| 1326 | T -> | No |
| 1408 | T -> G | No |
| 1450 | T -> G | No |
| 1660 | C -> | No |
| 1664 | C -> T | Yes |
| 1665 | T -> | No |
| 1797 | T -> | No |
| 1802 | T -> | No |
| 1913 | G -> A | Yes |
| 1985 | T -> | No |
| 2168 | A -> G | Yes |
| 2205 | T -> C | Yes |
| 2466 | A -> C | No |
| 2466 | A -> G | Yes |
| 2535 | T -> G | Yes |
| 2597 | T -> | No |
| 2648 | T -> C | No |
| 2720 | A -> T | No |
| 2738 | G -> A | Yes |
| 2782 | C -> T | Yes |
| 2790 | C -> | No |
| 2861 | C -> T | No |
| 2931 | T -> G | No |
| 3043 | T -> G | No |
| 3103 | T -> G | No |
| 3120 | T -> G | No |
| 3125 | T -> G | No |
| 3500 | A -> C | Yes |
| 3566 | A -> G | Yes |
| 5060 | G -> A | Yes |
| 5156 | -> T | No |
| 5533 | G -> C | No |
| 5789 | G -> A | Yes |
| 5866 | T -> C | Yes |
| 6591 | C -> T | Yes |
| 6619 | G -> A | Yes |
| 6905 | A -> T | Yes |
| 6922 | G -> C | Yes |
| 7046 | C -> G | Yes |
| 7319 | A -> G | Yes |
| 7706 | C -> T | Yes |
| 7894 | G -> A | Yes |
| 8099 | C -> G | Yes |
| 8324 | T -> C | No |
| 8555 | T -> C | Yes |
| 8627 | G -> T | Yes |
| 8644 | T -> C | Yes |
| 8704 | T -> C | No |
| 8781 | C -> T | Yes |
| 8787 | C -> T | Yes |
| 8827 | C -> T | No |
| 8847 | T -> C | No |
| 8847 | T -> G | No |
| 8909 | G -> A | No |
| 8947 | G -> A | Yes |
| 8960 | T -> C | Yes |
| 9096 | T -> C | No |
| 9096 | T -> G | No |
| 9207 | A -> G | No |
| 9424 | T -> C | Yes |
| 9516 | A -> | No |
| 9596 | C -> T | Yes |

Variant protein M77903_P5 (SEQ ID NO:609) according to the present invention has an amino acid sequence as given at the end of the application; it is encoded by transcript(s) M77903_T12 (SEQ ID NO:82). An alignment is given to the known protein (Translocon-associated protein (SEQ ID NO:641), alpha subunit precursor) at the end of the application. One or more alignments to one or more previously published protein sequences are given at the end of the application. A brief description of the relationship of the variant protein according to the present invention to each such aligned protein is as follows:

Comparison Report Between M77903_P5 (SEQ ID NO:609) and SSRA_HUMAN (SEQ ID NO:641):

1. An isolated chimeric polypeptide encoding for M77903_P5 (SEQ ID NO:609), comprising a first amino acid sequence being at least 90% homologous to MRLLPRLLLLLLLVFPATVLFRGGPRGLLAVAQDLTEDEETVEDSIIEDEDDEAEVEEDEPTDLVEDKEEED VSGEPEASPSADTTILFVKGEDFPANNIVKFLVGFTNKGTEDFIVESLDASFRYPQDYQFYIQNFTALPLNTV VPPQRQATFEYSFIPAEPMGGRPFGLVINLNYKDLNGNVFQDAVFNQTVTVIEREDGLDGET corresponding to amino acids 1-207 of SSRA_HUMAN (SEQ ID NO:641), which also corresponds to amino acids 1-207 of M77903_P5 (SEQ ID NO:609).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein M77903_P5 (SEQ ID NO:609) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein M77903_P5 (SEQ ID NO:609) sequence provides support for the deduced sequence of this variant protein according to the present invention).

TABLE 10
Amino acid mutations

| SNP position(s) | | |
|---|---|---|
| on amino acid | Alternative amino | Previously |
| sequence | acid(s) | known SNP? |
| 6 | R -> G | No |
| 24 | G -> | No |
| 28 | L -> S | Yes |
| 48 | E -> G | No |
| 54 | A -> T | Yes |
| 58 | E -> K | No |
| 63 | D -> | No |
| 89 | F -> | No |
| 116 | I -> M | No |
| 130 | Y -> H | No |
| 130 | Y -> N | No |
| 178 | K -> | No |

The glycosylation sites of variant protein M77903_P5 (SEQ ID NO:609), as compared to the known protein Translocon-associated protein (SEQ ID NO:641), alpha subunit precursor, are described in Table 11 (given according to their position(s) on the amino acid sequence in the first column; the second column indicates whether the glycosylation site is present in the variant protein; and the last column indicates whether the position is different on the variant protein).

TABLE 11
Glycosylation site(s)

| Position(s) on known | Present in | Position in |
|---|---|---|
| amino acid sequence | variant protein? | variant protein? |
| 191 | Yes | 191 |
| 136 | Yes | 136 |

Variant protein M77903_P5 (SEQ ID NO:609) is encoded by the following transcript(s): M77903_T12 (SEQ ID NO:82), for which the sequence(s) is/are given at the end of the application. The coding portion of transcript M77903_T12 (SEQ ID NO:82) is shown in bold; this coding portion starts at position 200 and ends at position 820. The transcript also has the following SNPs as listed in Table 12 (given according to their position on the nucleotide sequence, with the alternative nucleic acid listed; the last column indicates whether the SNP is known or not; the presence of known SNPs in variant protein M77903_P5 (SEQ ID NO:609) sequence provides support for the deduced sequence of this variant protein according to the present invention).

TABLE 12
Nucleic acid SNPs

| SNP position on | Alternative | Previously |
|---|---|---|
| nucleotide sequence | nucleic acid | known SNP? |
| 72 | G -> T | Yes |
| 120 | C -> G | No |
| 147 | G -> C | Yes |
| 215 | C -> G | No |
| 271 | C -> | No |
| 282 | T -> C | Yes |
| 342 | A -> G | No |
| 359 | G -> A | Yes |
| 371 | G -> A | No |
| 388 | T -> | No |
| 464 | T -> | No |
| 547 | T -> C | No |
| 547 | T -> G | No |
| 587 | T -> A | No |
| 587 | T -> C | No |
| 731 | A -> | No |
| 833 | A -> | No |
| 1023 | T -> | No |
| 1024 | G -> | No |
| 1202 | T -> A | Yes |
| 1230 | T -> | No |
| 1232 | T -> | No |
| 1314 | T -> G | No |
| 1356 | T -> G | No |
| 1566 | C -> | No |
| 1570 | C -> T | Yes |
| 1571 | T -> | No |
| 1703 | T -> | No |
| 1708 | T -> | No |
| 1819 | G -> A | Yes |
| 1891 | T -> | No |
| 2074 | A -> G | Yes |
| 2111 | T -> C | Yes |
| 2372 | A -> C | No |
| 2372 | A -> G | Yes |
| 2441 | T -> G | Yes |
| 2503 | T -> | No |
| 2554 | T -> C | No |
| 2626 | A -> T | No |
| 2644 | G -> A | Yes |
| 2688 | C -> T | Yes |
| 2696 | C -> | No |
| 2767 | C -> T | No |
| 2837 | T -> G | No |
| 2949 | T -> G | No |
| 3009 | T -> G | No |
| 3026 | T -> G | No |
| 3031 | T -> G | No |
| 3406 | A -> C | Yes |
| 3472 | A -> G | Yes |
| 4966 | G -> A | Yes |
| 5062 | -> T | No |
| 5439 | G -> C | No |
| 5695 | G -> A | Yes |
| 5772 | T -> C | Yes |
| 6497 | C -> T | Yes |
| 6525 | G -> A | Yes |
| 6811 | A -> T | Yes |
| 6828 | G -> C | Yes |
| 6952 | C -> G | Yes |
| 7225 | A -> G | Yes |
| 7612 | C -> T | Yes |
| 7800 | G -> A | Yes |
| 8005 | C -> G | Yes |
| 8230 | T -> C | No |
| 8461 | T -> C | Yes |
| 8533 | G -> T | Yes |
| 8550 | T -> C | Yes |
| 8610 | T -> C | No |
| 8687 | C -> T | Yes |
| 8693 | C -> T | Yes |
| 8733 | C -> T | No |
| 8753 | T -> C | No |
| 8753 | T -> G | No |
| 8815 | G -> A | No |
| 8853 | G -> A | Yes |
| 8866 | T -> C | Yes |
| 9002 | T -> C | No |
| 9002 | T -> G | No |
| 9113 | A -> G | No |
| 9330 | T -> C | Yes |
| 9422 | A -> | No |
| 9502 | C -> T | Yes |

Variant protein M77903_P15 (SEQ ID NO:610) according to the present invention has an amino acid sequence as given at the end of the application; it is encoded by transcript(s) M77903_T34 (SEQ ID NO:83). An alignment is given to the known protein (Translocon-associated protein (SEQ ID NO:641), alpha subunit precursor) at the end of the application. One or more alignments to one or more previously published protein sequences are given at the end of the application. A brief description of the relationship of the variant protein according to the present invention to each such aligned protein is as follows:

Comparison Report Between M77903_P15 (SEQ ID NO:610) and SSRA_HUMAN (SEQ ID NO:641):

1. An isolated chimeric polypeptide encoding for M77903_P15 (SEQ ID NO:610), comprising a first amino acid sequence being at least 90% homologous to MRLLPRLLLLLLLVFPATVLFRGGPRGLLAVAQDLTEDEETVEDSIIEDEDDEAEVEEDEPTDLVEDKEEED VSGEPEASPSADTTILFVKGEDFPANNIVKFLVGFTNKGTEDFIVESLDASFRYPQDYQFYIQNFTALPLNTV VPPQRQATFEYSFIPAEPMGGRPFGLVINLNYKDLN corresponding to amino acids 1-181 of SSRA_HUMAN (SEQ ID NO:641), which also corresponds to amino acids 1-181 of M77903_P15 (SEQ ID NO:610), and a second amino acid sequence being at least 70%, optionally at least 80%, preferably at least 85%, more preferably at least 90% and most preferably at least 95% homologous to a polypeptide having the sequence VRSSKPSFCLS (SEQ ID NO:1566) corresponding to amino acids 182-192 of M77903_P15 (SEQ ID NO:610), wherein said first amino acid sequence and second amino acid sequence are contiguous and in a sequential order.

2. An isolated polypeptide encoding for a tail of M77903_P15 (SEQ ID NO:610), comprising a polypeptide being at least 70%, optionally at least about 80%, preferably at least about 85%, more preferably at least about 90% and most preferably at least about 95% homologous to the sequence VRSSKPSFCLS (SEQ ID NO:1566) in M77903_P15 (SEQ ID NO:610).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein M77903_P15 (SEQ ID NO:610) also has the following non-silent SNPs (Single Nucleotide Polymorphisms) as listed in Table 13, (given according to their position(s) on the amino acid sequence, with the alternative amino acid(s) listed; the last column indicates whether the SNP is known or not; the presence of known SNPs in variant protein M77903_P15 (SEQ ID NO:610) sequence provides support for the deduced sequence of this variant protein according to the present invention).

TABLE 13
Amino acid mutations

| SNP position(s) on | Alternative | Previously |
|---|---|---|
| amino acid sequence | amino acid(s) | known SNP? |
| 6 | R -> G | No |
| 24 | G -> | No |
| 28 | L -> S | Yes |
| 48 | E -> G | No |
| 54 | A -> T | Yes |
| 58 | E -> K | No |
| 63 | D -> | No |
| 89 | F -> | No |
| 116 | I -> M | No |
| 130 | Y -> H | No |
| 130 | Y -> N | No |
| 178 | K -> | No |

The glycosylation sites of variant protein M77903_P15 (SEQ ID NO:610), as compared to the known protein Translocon-associated protein (SEQ ID NO:641), alpha subunit precursor, are described in Table 14 (given according to their position(s) on the amino acid sequence in the first column; the second column indicates whether the glycosylation site is present in the variant protein; and the last column indicates whether the position is different on the variant protein).

TABLE 14
Glycosylation site(s)

| Position(s) on known | Present in | Position in |
|---|---|---|
| amino acid sequence | variant protein? | variant protein? |
| 191 | No | |
| 136 | Yes | 136 |

Variant protein M77903_P15 (SEQ ID NO:610) is encoded by the following transcript(s): M77903_T34 (SEQ ID NO:83), for which the sequence(s) is/are given at the end of the application. The coding portion of transcript M77903_T34 (SEQ ID NO:83) is shown in bold; this coding portion starts at position 200 and ends at position 775. The transcript also has the following SNPs as listed in Table 15 (given according to their position on the nucleotide sequence, with the alternative nucleic acid listed; the last column indicates whether the SNP is known or not; the presence of known SNPs in variant protein M77903_P15 (SEQ ID NO:610) sequence provides support for the deduced sequence of this variant protein according to the present invention).

TABLE 15
Nucleic acid SNPs

| SNP position on | Alternative | Previously |
|---|---|---|
| nucleotide sequence | nucleic acid | known SNP? |
| 72 | G -> T | Yes |
| 120 | C -> G | No |
| 147 | G -> C | Yes |
| 215 | C -> G | No |
| 271 | C -> | No |
| 282 | T -> C | Yes |
| 342 | A -> G | No |
| 359 | G -> A | Yes |
| 371 | G -> A | No |
| 388 | T -> | No |
| 464 | T -> | No |
| 547 | T -> C | No |
| 547 | T -> G | No |
| 587 | T -> A | No |
| 587 | T -> C | No |
| 731 | A -> | No |

Variant protein M77903_P16 (SEQ ID NO:611) according to the present invention has an amino acid sequence as given at the end of the application; it is encoded by transcript(s) M77903_T36 (SEQ ID NO:84). An alignment is given to the known protein (Translocon-associated protein (SEQ ID NO:641), alpha subunit precursor) at the end of the application. One or more alignments to one or more previously published protein sequences are given at the end of the application. A brief description of the relationship of the variant protein according to the present invention to each such aligned protein is as follows:

Comparison Report Between M77903_P16 (SEQ ID NO:611) and SSRA_HUMAN (SEQ ID NO:641):

1. An isolated chimeric polypeptide encoding for M77903_P16 (SEQ ID NO:611), comprising a first amino acid sequence being at least 90% homologous to MRLLPRLLLLLLLVFPATVLFRGGPRGLLAVAQDLTEDEETVEDSIIEDEDDEAEVEEDEPTDLVEDKEEED VSGEPEASPSADTTILFVKGE corresponding to amino acids 1-93 of SSRA_HUMAN (SEQ ID NO:641) which also corresponds to amino acids 1-93 of M77903_P16 (SEQ ID NO:611), and a second amino acid sequence being at least 70%, optionally at least 80%, preferably at least 85%, more preferably at least 90% and most preferably at least 95% homologous to a polypeptide having the sequence GNTEVLVLIQM (SEQ ID NO:1567) corresponding to amino acids 94-104 of M77903_P16 (SEQ ID NO:611), wherein said first amino acid sequence and second amino acid sequence are contiguous and in a sequential order.

2. An isolated polypeptide encoding for a tail of M77903_P16 (SEQ ID NO:611), comprising a polypeptide being at least 70%, optionally at least about 80%, preferably at least about 85%, more preferably at least about 90% and most preferably at least about 95% homologous to the sequence GNTEVLVLIQM (SEQ ID NO:1567) in M77903_P16 (SEQ ID NO:611).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein M77903_P16 (SEQ ID NO:611) also has the following non-silent SNPs (Single Nucleotide Polymorphisms) as listed in Table 16, (given according to their position(s) on the amino acid sequence, with the alternative amino acid(s) listed; the last column indicates whether the SNP is known or not; the presence of known SNPs in variant protein M77903_P16 (SEQ ID NO:611) sequence provides support for the deduced sequence of this variant protein according to the present invention).

TABLE 16
Amino acid mutations

| SNP position(s) on | Alternative | Previously |
|---|---|---|
| amino acid sequence | amino acid(s) | known SNP? |
| 6 | R -> G | No |
| 24 | G -> | No |
| 28 | L -> S | Yes |
| 48 | E -> G | No |
| 54 | A -> T | Yes |
| 58 | E -> K | No |
| 63 | D -> | No |
| 89 | F -> | No |

The glycosylation sites of variant protein M77903_P16 (SEQ ID NO:611), as compared to the known protein Translocon-associated protein (SEQ ID NO:641), alpha subunit precursor, are described in Table 17 (given according to their position(s) on the amino acid sequence in the first column; the second column indicates whether the glycosylation site is present in the variant protein; and the last column indicates whether the position is different on the variant protein).

TABLE 17
Glycosylation site(s)

| Position(s) on known amino acid | |
|---|---|
| sequence | Present in variant protein? |
| 191 | No |
| 136 | No |

Variant protein M77903_P16 (SEQ ID NO:611) is encoded by the following transcript(s): M77903_T36 (SEQ ID NO:84), for which the sequence(s) is/are given at the end of the application. The coding portion of transcript M77903_T36 (SEQ ID NO:84) is shown in bold; this coding portion starts at position 200 and ends at position 511. The transcript also has the following SNPs as listed in Table 18 (given according to their position on the nucleotide sequence, with the alternative nucleic acid listed; the last column indicates whether the SNP is known or not; the presence of known SNPs in variant protein M77903_P16 (SEQ ID NO:611) sequence provides support for the deduced sequence of this variant protein according to the present invention).

TABLE 18
Nucleic acid SNPs

| SNP position on | Alternative | Previously |
|---|---|---|
| nucleotide sequence | nucleic acid | known SNP? |
| 72 | G -> T | Yes |
| 120 | C -> G | No |
| 147 | G -> C | Yes |
| 215 | C -> G | No |
| 271 | C -> | No |
| 282 | T -> C | Yes |
| 342 | A -> G | No |
| 359 | G -> A | Yes |
| 371 | G -> A | No |
| 388 | T -> | No |
| 464 | T -> | No |
| 527 | G -> A | Yes |
| 597 | C -> T | Yes |

As noted above, cluster M77903 features 29 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster M77903_node_{—}2 (SEQ ID NO:476) according to the present invention is supported by 150 libraries. The number of libraries was determined as previously described. This segment can be found in the following transcript(s): M77903_T11 (SEQ ID NO:81), M77903_T12 (SEQ ID NO:82), M77903_T34 (SEQ ID NO:83) and M77903_T36 (SEQ ID NO:84). Table 19 below describes the starting and ending position of this segment on each transcript.

TABLE 19
Segment location on transcripts

| | Segment | Segment |
|---|---|---|
| Transcript name | starting position | ending position |
| M77903_T11 (SEQ ID NO: 81) | 118 | 278 |
| M77903_T12 (SEQ ID NO: 82) | 118 | 278 |
| M77903_T34 (SEQ ID NO: 83) | 118 | 278 |
| M77903_T36 (SEQ ID NO: 84) | 118 | 278 |

Segment cluster M77903_node_{—}13 (SEQ ID NO:477) according to the present invention is supported by 1 libraries. The number of libraries was determined as previously described. This segment can be found in the following transcript(s): M77903_T36 (SEQ ID NO:84). Table 20 below describes the starting and ending position of this segment on each transcript.

TABLE 20
Segment location on transcripts

| | Segment | Segment |
|---|---|---|
| Transcript name | starting position | ending position |
| M77903_T36 (SEQ ID NO: 84) | 480 | 629 |

Segment cluster M77903_node_{—}16 (SEQ ID NO:478) according to the present invention is supported by 149 libraries. The number of libraries was determined as previously described. This segment can be found in the following transcript(s): M77903_T11 (SEQ ID NO:81), M77903_T12 (SEQ ID NO:82) and M77903_T34 (SEQ ID NO:83). Table 21 below describes the starting and ending position of this segment on each transcript.

TABLE 21
Segment location on transcripts

| | Segment | Segment |
|---|---|---|
| Transcript name | starting position | ending position |
| M77903_T11 (SEQ ID NO: 81) | 523 | 683 |
| M77903_T12 (SEQ ID NO: 82) | 523 | 683 |
| M77903_T34 (SEQ ID NO: 83) | 523 | 683 |

Segment cluster M77903_node_{—}18 (SEQ ID NO:479) according to the present invention is supported by 2 libraries. The number of libraries was determined as previously described. This segment can be found in the following transcript(s): M77903_T34 (SEQ ID NO:83). Table 22 below describes the starting and ending position of this segment on each transcript.

TABLE 22
Segment location on transcripts

| | Segment | Segment |
|---|---|---|
| Transcript name | starting position | ending position |
| M77903_T34 (SEQ ID NO: 83) | 743 | 935 |

Segment cluster M77903_node_{—}35 (SEQ ID NO:480) according to the present invention is supported by 145 libraries. The number of libraries was determined as previously described. This segment can be found in the following transcript(s): M77903_T11 (SEQ ID NO:81) and M77903_T12 (SEQ ID NO:82). Table 23 below describes the starting and ending position of this segment on each transcript.

TABLE 23
Segment location on transcripts

| | Segment | Segment |
|---|---|---|
| Transcript name | starting position | ending position |
| M77903_T11 (SEQ ID NO: 81) | 978 | 1162 |
| M77903_T12 (SEQ ID NO: 82) | 884 | 1068 |

Segment cluster M77903_node_{—}36 (SEQ ID NO:481) according to the present invention is supported by 173 libraries. The number of libraries was determined as previously described. This segment can be found in the following transcript(s): M77903_T11 (SEQ ID NO:81) and M77903_T12 (SEQ ID NO:82). Table 24 below describes the starting and ending position of this segment on each transcript.

TABLE 24
Segment location on transcripts

| | Segment | Segment |
|---|---|---|
| Transcript name | starting position | ending position |
| M77903_T11 (SEQ ID NO: 81) | 1163 | 1571 |
| M77903_T12 (SEQ ID NO: 82) | 1069 | 1477 |

Segment cluster M77903_node_{—}37 (SEQ ID NO:482) according to the present invention is supported by 128 libraries. The number of libraries was determined as previously described. This segment can be found in the following transcript(s): M77903_T11 (SEQ ID NO:81) and M77903_T12 (SEQ ID NO:82). Table 25 below describes the starting and ending position of this segment on each transcript.

TABLE 25
Segment location on transcripts

| | Segment | Segment |
|---|---|---|
| Transcript name | starting position | ending position |
| M77903_T11 (SEQ ID NO: 81) | 1572 | 1913 |
| M77903_T12 (SEQ ID NO: 82) | 1478 | 1819 |

Segment cluster M77903_node_{—}38 (SEQ ID NO:483) according to the present invention is supported by 152 libraries. The number of libraries was determined as previously described. This segment can be found in the following transcript(s): M77903_T11 (SEQ ID NO:81) and M77903_T12 (SEQ ID NO:82). Table 26 below describes the starting and ending position of this segment on each transcript.

TABLE 26
Segment location on transcripts

| | Segment | Segment |
|---|---|---|
| Transcript name | starting position | ending position |
| M77903_T11 (SEQ ID NO: 81) | 1914 | 2411 |
| M77903_T12 (SEQ ID NO: 82) | 1820 | 2317 |

Segment cluster M77903_node_{—}40 (SEQ ID NO:484) according to the present invention is supported by 186 libraries. The number of libraries was determined as previously described. This segment can be found in the following transcript(s): M77903_T11 (SEQ ID NO:81) and M77903_T12 (SEQ ID NO:82). Table 27 below describes the starting and ending position of this segment on each transcript.

TABLE 27
Segment location on transcripts

| | Segment | Segment |
|---|---|---|
| Transcript name | starting position | ending position |
| M77903_T11 (SEQ ID NO: 81) | 2412 | 2923 |
| M77903_T12 (SEQ ID NO: 82) | 2318 | 2829 |

Segment cluster M77903_node_{—}44 (SEQ ID NO:485) according to the present invention is supported by 122 libraries. The number of libraries was determined as previously described. This segment can be found in the following transcript(s): M77903_T11 (SEQ ID NO:81) and M77903_T12 (SEQ ID NO:82). Table 28 below describes the starting and ending position of this segment on each transcript.

TABLE 28
Segment location on transcripts

| | Segment | Segment |
|---|---|---|
| Transcript name | starting position | ending position |
| M77903_T11 (SEQ ID NO: 81) | 3079 | 3826 |
| M77903_T12 (SEQ ID NO: 82) | 2985 | 3732 |

Segment cluster M77903_node_{—}46 (SEQ ID NO:486) according to the present invention is supported by 10 libraries. The number of libraries was determined as previously described. This segment can be found in the following transcript(s): M77903_T11 (SEQ ID NO:81) and M77903_T12 (SEQ ID NO:82). Table 29 below describes the starting and ending position of this segment on each transcript.

TABLE 29
Segment location on transcripts

| | Segment | Segment |
|---|---|---|
| Transcript name | starting position | ending position |
| M77903_T11 (SEQ ID NO: 81) | 3827 | 4196 |
| M77903_T12 (SEQ ID NO: 82) | 3733 | 4102 |

Segment cluster M77903_node_{—}47 (SEQ ID NO:487) according to the present invention is supported by 40 libraries. The number of libraries was determined as previously described. This segment can be found in the following transcript(s): M77903_T11 (SEQ ID NO:81) and M77903_T12 (SEQ ID NO:82). Table 30 below describes the starting and ending position of this segment on each transcript.

TABLE 30
Segment location on transcripts

| | Segment | Segment |
|---|---|---|
| Transcript name | starting position | ending position |
| M77903_T11 (SEQ ID NO: 81) | 4197 | 5182 |
| M77903_T12 (SEQ ID NO: 82) | 4103 | 5088 |

Segment cluster M77903_node_{—}48 (SEQ ID NO:488) according to the present invention is supported by 63 libraries. The number of libraries was determined as previously described. This segment can be found in the following transcript(s): M77903_T11 (SEQ ID NO:81) and M77903_T12 (SEQ ID NO:82). Table 31 below describes the starting and ending position of this segment on each transcript.

TABLE 31
Segment location on transcripts

| | Segment | Segment |
|---|---|---|
| Transcript name | starting position | ending position |
| M77903_T11 (SEQ ID NO: 81) | 5183 | 6133 |
| M77903_T12 (SEQ ID NO: 82) | 5089 | 6039 |

Segment cluster M77903_node_{—}49 (SEQ ID NO:489) according to the present invention is supported by 7 libraries. The number of libraries was determined as previously described. This segment can be found in the following transcript(s): M77903_T11 (SEQ ID NO:81) and M77903_T12 (SEQ ID NO:82). Table 32 below describes the starting and ending position of this segment on each transcript.

TABLE 32
Segment location on transcripts

| | Segment | Segment |
|---|---|---|
| Transcript name | starting position | ending position |
| M77903_T11 (SEQ ID NO: 81) | 6134 | 6319 |
| M77903_T12 (SEQ ID NO: 82) | 6040 | 6225 |

Segment cluster M77903_node_{—}51 (SEQ ID NO:490) according to the present invention is supported by 14 libraries. The number of libraries was determined as previously described. This segment can be found in the following transcript(s): M77903_T11 (SEQ ID NO:81) and M77903_T12 (SEQ ID NO:82). Table 33 below describes the starting and ending position of this segment on each transcript.

TABLE 33
Segment location on transcripts

| | Segment | Segment |
|---|---|---|
| Transcript name | starting position | ending position |
| M77903_T11 (SEQ ID NO: 81) | 6320 | 7542 |
| M77903_T12 (SEQ ID NO: 82) | 6226 | 7448 |

Segment cluster M77903_node_{—}52 (SEQ ID NO:491) according to the present invention is supported by 160 libraries. The number of libraries was determined as previously described. This segment can be found in the following transcript(s): M77903_T11 (SEQ ID NO:81) and M77903_T12 (SEQ ID NO:82). Table 34 below describes the starting and ending position of this segment on each transcript.

TABLE 34
Segment location on transcripts

| | Segment | Segment |
|---|---|---|
| Transcript name | starting position | ending position |
| M77903_T11 (SEQ ID NO: 81) | 7543 | 9702 |
| M77903_T12 (SEQ ID NO: 82) | 7449 | 9608 |

According to an optional embodiment of the present invention, short segments related to the above cluster are also provided. These segments are up to about 120 bp in length, and so are included in a separate description.

Segment cluster M77903_node_{—}1 (SEQ ID NO:492) according to the present invention is supported by 4 libraries. The number of libraries was determined as previously described. This segment can be found in the following transcript(s): M77903_T11 (SEQ ID NO:81), M77903_T12 (SEQ ID NO:82), M77903_T34 (SEQ ID NO:83) and M77903_T36 (SEQ ID NO:84). Table 35 below describes the starting and ending position of this segment on each transcript.

TABLE 35
Segment location on transcripts

| | Segment | Segment |
|---|---|---|
| Transcript name | starting position | ending position |
| M77903_T11 (SEQ ID NO: 81) | 1 | 117 |
| M77903_T12 (SEQ ID NO: 82) | 1 | 117 |
| M77903_T34 (SEQ ID NO: 83) | 1 | 117 |
| M77903_T36 (SEQ ID NO: 84) | 1 | 117 |

Segment cluster M77903_node_{—}5 (SEQ ID NO:493) according to the present invention is supported by 154 libraries. The number of libraries was determined as previously described. This segment can be found in the following transcript(s): M77903_T11 (SEQ ID NO:81), M77903_T12 (SEQ ID NO:82), M77903_T34 (SEQ ID NO:83) and M77903_T36 (SEQ ID NO:84). Table 36 below describes the starting and ending position of this segment on each transcript.

TABLE 36
Segment location on transcripts

| | Segment | Segment |
|---|---|---|
| Transcript name | starting position | ending position |
| M77903_T11 (SEQ ID NO: 81) | 279 | 391 |
| M77903_T12 (SEQ ID NO: 82) | 279 | 391 |
| M77903_T34 (SEQ ID NO: 83) | 279 | 391 |
| M77903_T36 (SEQ ID NO: 84) | 279 | 391 |

Segment cluster M77903_node_{—}9 (SEQ ID NO:494) according to the present invention can be found in the following transcript(s): M77903_T11 (SEQ ID NO:81), M77903_T12 (SEQ ID NO:82), M77903_T34 (SEQ ID NO:83) and M77903_T36 (SEQ ID NO:84). Table 37 below describes the starting and ending position of this segment on each transcript.

TABLE 37
Segment location on transcripts

| | Segment | Segment |
|---|---|---|
| Transcript name | starting position | ending position |
| M77903_T11 (SEQ ID NO: 81) | 392 | 395 |
| M77903_T12 (SEQ ID NO: 82) | 392 | 395 |
| M77903_T34 (SEQ ID NO: 83) | 392 | 395 |
| M77903_T36 (SEQ ID NO: 84) | 392 | 395 |

Segment cluster M77903_node_{—}10 (SEQ ID NO:495) according to the present invention is supported by 148 libraries. The number of libraries was determined as previously described. This segment can be found in the following transcript(s): M77903_T11 (SEQ ID NO:81), M77903_T12 (SEQ ID NO:82), M77903_T34 (SEQ ID NO:83) and M77903_T36 (SEQ ID NO:84). Table 38 below describes the starting and ending position of this segment on each transcript.

TABLE 38
Segment location on transcripts

| | Segment | Segment |
|---|---|---|
| Transcript name | starting position | ending position |
| M77903_T11 (SEQ ID NO: 81) | 396 | 468 |
| M77903_T12 (SEQ ID NO: 82) | 396 | 468 |
| M77903_T34 (SEQ ID NO: 83) | 396 | 468 |
| M77903_T36 (SEQ ID NO: 84) | 396 | 468 |

Segment cluster M77903_node_{—}11 (SEQ ID NO:496) according to the present invention can be found in the following transcript(s): M77903_T11 (SEQ ID NO:81), M77903_T12 (SEQ ID NO:82), M77903_T34 (SEQ ID NO:83) and M77903_T36 (SEQ ID NO:84). Table 39 below describes the starting and ending position of this segment on each transcript.

TABLE 39
Segment location on transcripts

| | Segment | Segment |
|---|---|---|
| Transcript name | starting position | ending position |
| M77903_T11 (SEQ ID NO: 81) | 469 | 473 |
| M77903_T12 (SEQ ID NO: 82) | 469 | 473 |
| M77903_T34 (SEQ ID NO: 83) | 469 | 473 |
| M77903_T36 (SEQ ID NO: 84) | 469 | 473 |

Segment cluster M77903_node_{—}12 (SEQ ID NO:497) according to the present invention can be found in the following transcript(s): M77903_T11 (SEQ ID NO:81), M77903_T12 (SEQ ID NO:82), M77903_T34 (SEQ ID NO:83) and M77903_T36 (SEQ ID NO:84). Table 40 below describes the starting and ending position of this segment on each transcript.

TABLE 40
Segment location on transcripts

| | Segment | Segment |
|---|---|---|
| Transcript name | starting position | ending position |
| M77903_T11 (SEQ ID NO: 81) | 474 | 479 |
| M77903_T12 (SEQ ID NO: 82) | 474 | 479 |
| M77903_T34 (SEQ ID NO: 83) | 474 | 479 |
| M77903_T36 (SEQ ID NO: 84) | 474 | 479 |

Segment cluster M77903_node_{—}15 (SEQ ID NO:498) according to the present invention is supported by 129 libraries. The number of libraries was determined as previously described. This segment can be found in the following transcript(s): M77903_T11 (SEQ ID NO:81), M77903_T12 (SEQ ID NO:82) and M77903_T34 (SEQ ID NO:83). Table 41 below describes the starting and ending position of this segment on each transcript.

TABLE 41
Segment location on transcripts

| | Segment | Segment |
|---|---|---|
| Transcript name | starting position | ending position |
| M77903_T11 (SEQ ID NO: 81) | 480 | 522 |
| M77903_T12 (SEQ ID NO: 82) | 480 | 522 |
| M77903_T34 (SEQ ID NO: 83) | 480 | 522 |

Segment cluster M77903_node_{—}17 (SEQ ID NO:499) according to the present invention is supported by 141 libraries. The number of libraries was determined as previously described. This segment can be found in the following transcript(s): M77903_T11 (SEQ ID NO:81), M77903_T12 (SEQ ID NO:82) and M77903_T34 (SEQ ID NO:83). Table 42 below describes the starting and ending position of this segment on each transcript.

TABLE 42
Segment location on transcripts

| | Segment | Segment |
|---|---|---|
| Transcript name | starting position | ending position |
| M77903_T11 (SEQ ID NO: 81) | 684 | 742 |
| M77903_T12 (SEQ ID NO: 82) | 684 | 742 |
| M77903_T34 (SEQ ID NO: 83) | 684 | 742 |

Segment cluster M77903_node_{—}20 (SEQ ID NO:500) according to the present invention is supported by 134 libraries. The number of libraries was determined as previously described. This segment can be found in the following transcript(s): M77903_T11 (SEQ ID NO:81) and M77903_T12 (SEQ ID NO:82). Table 43 below describes the starting and ending position of this segment on each transcript.

TABLE 43
Segment location on transcripts

| | Segment | Segment |
|---|---|---|
| Transcript name | starting position | ending position |
| M77903_T11 (SEQ ID NO: 81) | 743 | 819 |
| M77903_T12 (SEQ ID NO: 82) | 743 | 819 |

Segment cluster M77903_node_{—}28 (SEQ ID NO:501) according to the present invention is supported by 134 libraries. The number of libraries was determined as previously described. This segment can be found in the following transcript(s): M77903_T11 (SEQ ID NO:81). Table 44 below describes the starting and ending position of this segment on each transcript.

TABLE 44
Segment location on transcripts

| | Segment | Segment |
|---|---|---|
| Transcript name | starting position | ending position |
| M77903_T11 (SEQ ID NO: 81) | 820 | 913 |

Segment cluster M77903_node_{—}34 (SEQ ID NO:502) according to the present invention is supported by 134 libraries. The number of libraries was determined as previously described. This segment can be found in the following transcript(s): M77903_T11 (SEQ ID NO:81) and M77903_T12 (SEQ ID NO:82). Table 45 below describes the starting and ending position of this segment on each transcript.

TABLE 45
Segment location on transcripts

| | Segment | Segment |
|---|---|---|
| Transcript name | starting position | ending position |
| M77903_T11 (SEQ ID NO: 81) | 914 | 977 |
| M77903_T12 (SEQ ID NO: 82) | 820 | 883 |

Segment cluster M77903_node_{—}41 (SEQ ID NO:503) according to the present invention is supported by 119 libraries. The number of libraries was determined as previously described. This segment can be found in the following transcript(s): M77903_T11 (SEQ ID NO:81) and M77903_T12 (SEQ ID NO:82). Table 46 below describes the starting and ending position of this segment on each transcript.

TABLE 46
Segment location on transcripts

| | Segment | Segment |
|---|---|---|
| Transcript name | starting position | ending position |
| M77903_T11 (SEQ ID NO: 81) | 2924 | 2984 |
| M77903_T12 (SEQ ID NO: 82) | 2830 | 2890 |

Segment cluster M77903_node_{—}42 (SEQ ID NO:504) according to the present invention is supported by 123 libraries. The number of libraries was determined as previously described. This segment can be found in the following transcript(s): M77903_T11 (SEQ ID NO:81) and M77903_T12 (SEQ ID NO:82). Table 47 below describes the starting and ending position of this segment on each transcript.

TABLE 47
Segment location on transcripts

| | Segment | Segment |
|---|---|---|
| Transcript name | starting position | ending position |
| M77903_T11 (SEQ ID NO: 81) | 2985 | 3078 |
| M77903_T12 (SEQ ID NO: 82) | 2891 | 2984 |

### Variant Protein Alignment to the Previously Known Protein:

Sequence name: SSRA_HUMAN (SEQ ID NO:641)
Sequence documentation:
Alignment of: M77903_P4 (SEQ ID NO:608) × SSRA_HUMAN (SEQ ID NO:641) ..
Alignment segment 1/1:

| | | | |
|---|---|---|---|
| Quality: | 1991.00 | Escore: | 0 |
| Matching length: | 208 | Total length: | 208 |
| Matching Percent Similarity: | 100.00 | Matching Percent Identity: | 99.52 |
| Total Percent Similarity: | 100.00 | Total Percent Identity: | 99.52 |
| Gaps: | 0 | | |
| Alignment: | | | |

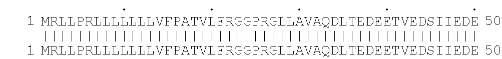

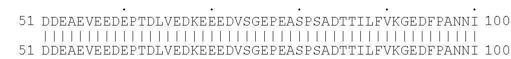

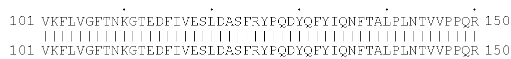

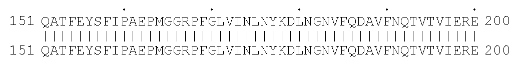

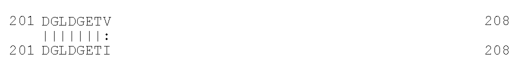

Sequence name: SSRA_HUMAN (SEQ ID NO:641)
Sequence documentation:
Alignment of: M77903_P5 (SEQ ID NO:609) × SSRA_HUMAN (SEQ ID NO:641) ..
Alignment segment 1/1:

| | | | |
|---|---|---|---|
| Quality: | 1987.00 | Escore: | 0 |
| Matching length: | 207 | Total length: | 207 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

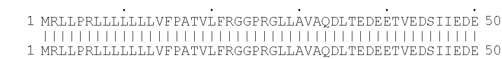

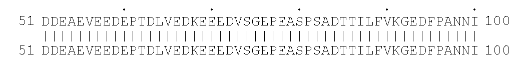

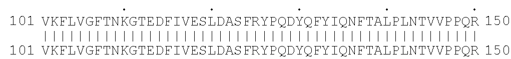

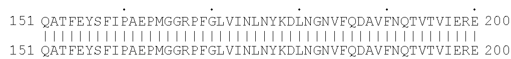

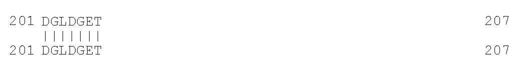

Sequence name: SSRA_HUMAN (SEQ ID NO:641)
Sequence documentation:
Alignment of: M77903_P15 (SEQ ID NO:610) × SSRA_HUMAN (SEQ ID 110:641) ..
Alignment segment 1/1:

| | | | |
|---|---|---|---|
| Quality: | 1741.00 | Escore: | 0 |
| Matching length: | 181 | Total length: | 181 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

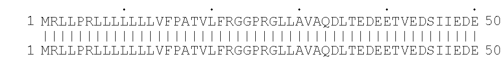

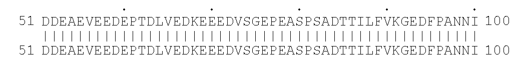

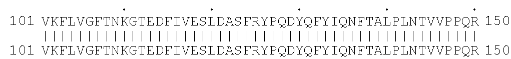

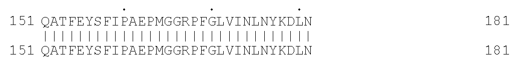

Sequence name: SSRA_HUMAN (SEQ ID NO:641)
Sequence documentation:
Alignment of: M77903_P16 (SEQ ID NO:611) × SSRA_HUMAN (SEQ ID NO:641) ..
Alignment segment 1/1:

| | | | |
|---|---|---|---|
| Quality: | 869.00 | Escore: | 0 |
| Matching length: | 93 | Total length: | 93 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

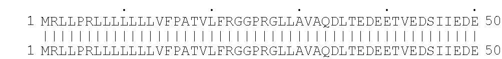

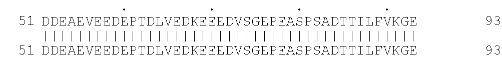

Expression of SSRA_HUMAN: SSR-Alpha M77903 Transcripts, which are Detectable by Amplicon, as Depicted in Sequence Name M77903Seg18 (SEQ ID NO: 1303) in Normal and Cancerous Colon Tissues.

Transcripts detectable by or according to M77903seg18 amplicon (SEQ ID NO:1303) and M77903seg18F (SEQ ID NO: 1301) and M77903seg18R (SEQ ID NO: 1302) primers were measured by real time PCR. In parallel the expression of four housekeeping genes: PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), and, G6PD (GenBank Accession No. NM_{—}000402 (SEQ ID NO:1578); HPRT1-amplicon, SEQ ID NO:615), and RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1 Tissue samples in testing panel), to obtain a value of fold up-regulation for each sample relative to median of the normal PM samples.

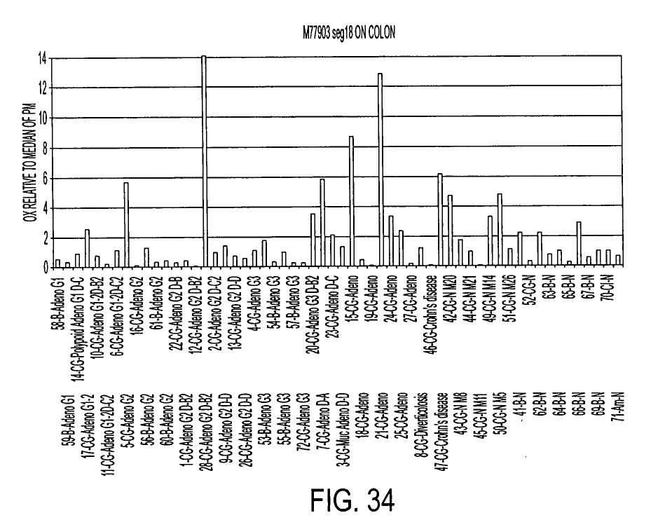

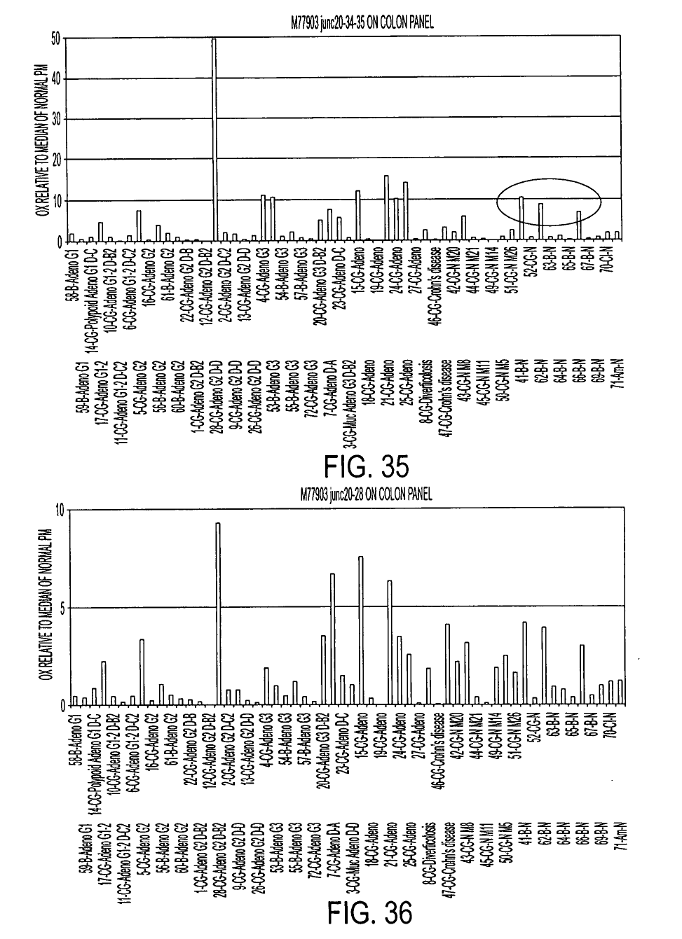

FIG. 34 is a histogram showing over expression of the above-indicated SSRA_HUMAN: SSR-alpha transcripts in cancerous colon samples relative to the normal samples.

As is evident from FIG. 34, the expression of SSRA_HUMAN: SSR-alpha transcripts detectable by the above amplicon(s) in a few cancer samples was higher than in the non-cancerous samples (Sample Nos. 41, 52, 62-67, 69-71 Table 1 Tissue samples in testing panel). Notably an over-expression of at least 5 fold was found in 5 out of 37 adenocarcinoma samples.

Primer pairs are also optionally and preferably encompassed within the present invention; for example, for the above experiment, the following primer pair was used as a non-limiting illustrative example only of a suitable primer pair: M77903seg18F forward primer (SEQ ID NO: 1301); and M77903seg18R reverse primer (SEQ ID NO: 1302).

The present invention also preferably encompasses any amplicon obtained through the use of any suitable primer pair; for example, for the above experiment, the following amplicon was obtained as a non-limiting illustrative example only of a suitable amplicon: M77903seg18] (SEQ ID NO: 1303).

M77903seg18F

(SEQ ID NO: 1301)

CGGTGACGTTGTTTAATAGAATATATCTGT
M77903seg18R

(SEQ ID NO: 1302)

AAGAAACGTGCAATTTATCTTTGCT
M77903seg18 amplicon

(SEQ ID NO: 1303)

CGGTGACGTTGTTTAATAGAATATATCTGTTCATTCAGTTGCCTGTTTTG
TGGTTGAACCTGTGATAGCCACCAGGGAAGCAAAGATAAATTGCACGTTT
CTT

As can be seen from FIGS. 35 and 36, for cluster M77903, amplicon name: M77903 junc20-34-35, and M77903 junc20-28, respectively, low over expression was observed in one experiment carried out with colon.

### Expression of SSRA_HUMAN

### Translocon-Associated Protein, Alpha Subunit (Trap-Alpha Signal Sequence Receptor Alpha SubunitSSR-Alpha) M77903 Transcripts which are Detectable by Amplicon as Depicted in Sequence Name M77903junc20-28 (SEQ ID NO: 1306) in Normal and Cancerous Colon Tissues

Expression of SSRA_HUMAN: Translocon-associated protein, alpha subunit (TRAP-alpha Signal sequence receptor alpha subunitSSR-alpha ) transcripts detectable by or according to junc20-28, M77903junc20-28 amplicon (SEQ ID NO: 1306) and primers M77903junc20-28F (SEQ ID NO: 1304) and M77903junc20-28R (SEQ ID NO: 1305) was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); HPRT1-amplicon, SEQ ID NO:615), RPS27A (GenBank Accession No. NM_{—}002954 (SEQ ID NO:1579); RPS27A amplicon, SEQ ID NO:1261) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

FIG. 35 is a histogram showing over expression of the above-indicated SSRA_HUMAN: Translocon-associated protein, alpha subunit TRAP-alpha Signal sequence receptor alpha subunitSSR-alpha transcripts in cancerous colon samples relative to the normal samples.

As is evident from FIG. 35, the expression of the above-indicated SSRA_HUMAN: Translocon-associated protein, alpha subunit TRAP-alpha Signal sequence receptor alpha subunitSSR-alpha transcripts detectable by the above amplicon in cancer samples was higher in a few samples than in the non-cancerous samples (Sample Nos. 41, 52, 62-67, 69-71, Table 1, above, “Tissue samples in testing panel”). Notably an over-expression of at least 5 fold was found in 4 out of 36 adenocarcinoma samples.

Primer pairs are also optionally and preferably encompassed within the present invention; for example, for the above experiment, the following primer pair was used as a non-limiting illustrative example only of a suitable primer pair: M77903junc20-28F forward primer (SEQ ID NO: 1304); and M77903junc20-28R reverse primer (SEQ ID NO: 1305).

The present invention also preferably encompasses any amplicon obtained through the use of any suitable primer pair; for example, for the above experiment, the following amplicon was obtained as a non-limiting illustrative example only of a suitable amplicon: M77903junc20-28 (SEQ ID NO: 1306).

Primers:
Forward primer M77903junc20-28F (SEQ ID NO: 1304):
GGCAATGTATTCCAAGATGCAG
Reverse primer M77903junc20-28R (SEQ ID NO: 1305):
TCTGTATGGGTCTCTTACGGTTTCT
Amplicon M77903junc20-28 (SEQ ID NO: 1306):
GGCAATGTATTCCAGATGCAGTCTTCAATCAAACAGTTACAGTTATTGAA
AGAGAGGATGGGTTAGATGGAGAAACCGTAAGAGACCCATACAGA

### Expression of SSRA_HUMAN

### Translocon-Associated Protein, Alpha Subunit Trap-Alpha Signal Sequence Receptor Alpha SubunitSSR-AlphaM77903 Transcripts which are Detectable by Amplicon as Depicted in Sequence Name M77903junc20-34-35 (SEQ ID NO: 1309) in Normal and Cancerous Colon Tissues

Expression of SSRA_HUMAN: Translocon-associated protein, alpha subunit TRAP-alpha Signal sequence receptor alpha subunitSSR-alpha transcripts detectable by or according to junc20-34-35, M77903junc20-34-35 amplicon (SEQ ID NO: 1309) and primers M77903junc20-34-35F (SEQ ID NO: 1307) and M77903junc20-34-35R (SEQ ID NO: 1308) was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); HPRT1-amplicon, SEQ ID NO:615), RPS27A (GenBank Accession No. NM_{—}002954 (SEQ ID NO:1579); RPS27A amplicon, SEQ ID NO:1261) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71 Table 1, above, “Tissue samples in testing panel”), to obtain a value of fold up-regulation for each sample relative to median of the normal PM samples.

FIG. 36 is a histogram showing over expression of the above-indicated SSRA_HUMAN: Translocon-associated protein, alpha subunit TRAP-alpha Signal sequence receptor alpha subunit SSR-alpha transcripts in cancerous colon samples relative to the normal samples.

As is evident from FIG. 36, the expression of SSRA_HUMAN: Translocon-associated protein, alpha subunit, TRAP-alpha Signal sequence receptor alpha subunitSSR-alpha transcripts detectable by the above amplicon in cancer samples was higher in a few samples than in the non-cancerous samples (Sample Nos. 41, 52, 62-67, 69-71, Table 1, above, “Tissue samples in testing panel”). Notably an over-expression of at least 10 fold was found in 7 out of 36 adenocarcinoma samples.

Primer pairs are also optionally and preferably encompassed within the present invention; for example, for the above experiment, the following primer pair was used as a non-limiting illustrative example only of a suitable primer pair: M77903junc20-34-35F forward primer (SEQ ID NO: 1307); and M77903junc20-34-35R reverse primer (SEQ ID NO: 1308).

The present invention also preferably encompasses any amplicon obtained through the use of any suitable primer pair; for example, for the above experiment, the following amplicon was obtained as a non-limiting illustrative example only of a suitable amplicon: M77903junc20-34-35 (SEQ ID NO: 1309).

Primers:
Forward primer M77903junc20-34-35F (SEQ ID NO:
1307):
ATGGGTTAGATGGAGAAACATAAAGCT
Reverse primer M77903junc20-34-35R (SEQ ID NO:
1308):
TGCACAAAGGAACATTTACTCATCA
Amplicon M77903junc20-34-35 (SEQ ID NO: 1309):
ATGGGTTAGATGGAGAAACATAAAGCTTCACCAAGAAGGTTGCCCAGGAA
ACGGGCACAGAAGAGATCAGTGGGATCTGATGAGTAAATGTTCCTTTGTG
CA

Combined expression of 6 Sequences (M85491seg24 (SEQ ID NO: 1276), M77903 seg18 (SEQ ID NO: 1303), M77903junc20-28 (SEQ ID NO: 1306), Z44808 junc8-11 (SEQ ID NO: 1291), Z25299 seg 20 (SEQ ID NO: 1294) and HSKITCR seg3 (SEQ ID NO: 1309)) in Normal and Cancerous Colon Tissues

Expression of Ephrin type-B receptor 2 precursor (EC 2.7.1.112) (Tyrosine-protein kinase receptor EPH-3), SSRA_HUMAN, SMO2_HUMAN SPARC related modular calcium-binding protein 2 precursor (Secreted modular calcium-binding protein 2) (SMOC-2) (Smooth muscle-associated protein 2), Secretory leukocyte protease inhibitor Acid-stable proteinase inhibitor and KIT_HUMAN; mast/stem cell growth factor receptor SCFR; Proto-oncogene tyrosine-protein kinase Kit; v-kit; CD117 antigen transcripts detectable by or according to M85491seg24 (SEQ ID NO: 1276), M77903 seg18 (SEQ ID NO: 1303), M77903junc20-28 (SEQ ID NO: 1306), Z44808 junc8-11 (SEQ ID NO: 1291), Z25299 seg 20 (SEQ ID NO: 1294) and HSKITCR seg3 (SEQ ID NO: 1309) amplicons and M85491seg24F (SEQ ID NO: 1274), M85491seg24R (SEQ ID NO: 1275), M77903 seg18F (SEQ ID NO: 1301), M77903 seg18R (SEQ ID NO: 1302), M77903junc20-28F (SEQ ID NO: 1304), M77903junc20-28R (SEQ ID NO: 1305), Z44808 junc8-11 (SEQ ID NO: 1289), Z44808 junc8-11R (SEQ ID NO: 1290), Z25299 seg 20F (SEQ ID NO: 1292), Z25299 seg 20R (SEQ ID NO: 1293), HSKITCR seg3F (SEQ ID NO: 1307) and HSKITCR seg3R (SEQ ID NO: 1308) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon —HPRT1-amplicon, SEQ ID NO:612), G6PD (GenBank Accession No. NM_{—}000402 (SEQ ID NO:1578); HPRT1-amplicon, SEQ ID NO:615) and RPS27A (GenBank Accession No. NM_{—}002954 (SEQ ID NO:1579); RPS27A amplicon, SEQ ID NO:1261) was measured similarly. For each RT sample, the expression of the above amplicons was normalized to the geometric mean of the quantities of the housekeeping genes. The normalized quantity of each RT sample of each amplicon was then divided by the median of the quantities of the normal post-mortem (PM) samples detected for the same amplicon (Sample Nos. 41, 52, 62-67, 69-71 Table 3, above), to obtain a value of fold up-regulation for each sample relative to median of the normal PM samples. The reciprocal of this ratio was calculated for HSKITCR seg3 (SEQ ID NO: 1309), to obtain a value of fold down-regulation for each sample relative to median of the normal PM samples. The expression of HSKITCR transcripts which can be detected by the HSKITCR seg3 (SEQ ID NO: 1309), is described also in the patent application “NOVEL NUCLEOTIDE AND AMINO ACID SEQUENCES, AND ASSAYS AND METHODS OF USE THEREOF FOR DIAGNOSIS”, attorney reference number XXXXX, by the same inventors, filed on the same date ans incorporated herein by reference.

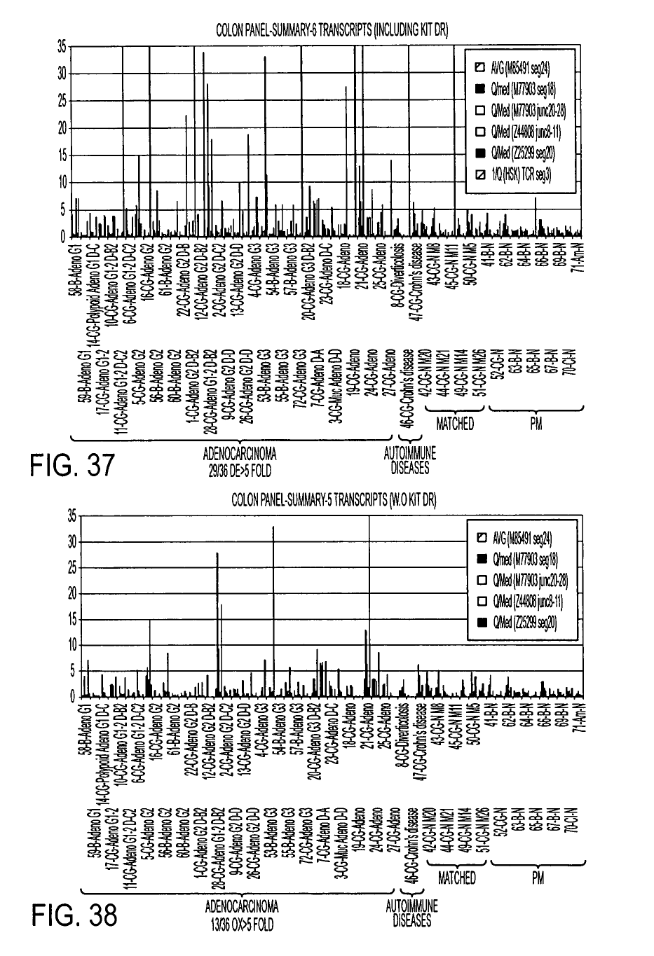

FIGS. 37-38 are histograms showing differential expression of the above-indicated transcripts in cancerous colon samples relative to the normal samples, in different combinations. The number and percentage of samples that exhibit at least 5 fold differential of at least one of the sequences, out of the total number of samples tested is indicated in the bottom.

As is evident from FIGS. 37-38, differential expression of at least 5 fold in at least one of the sequences was found in 29 out of 36 adenocarcinoma samples in the combinations of 6 transcripts, and in 13 out of 36 adenocarcinoma samples in the combinations of 5 transcripts.

Statistical analysis was applied to verify the significance of these results, as described below. Threshold of 5 fold differential expression of at least one of the amplicons was found to differentiate between cancer and normal samples as checked by exact fisher test.

The above values demonstrate statistical significance of the results.

### Description for Cluster HSSTROL3

Cluster HSSTROL3 features 6 transcript(s) and 16 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| HSSTROL3_T5 | 85 |
| HSSTROL3_T8 | 86 |
| HSSTROL3_T9 | 87 |
| HSSTROL3_T10 | 88 |
| HSSTROL3_T11 | 505 |
| HSSTROL3_T12 | 506 |

TABLE 2
Segments of interest

| Segment Name | SEQ ID NO: |
|---|---|
| HSSTROL3_node_6 | 507 |
| HSSTROL3_node_10 | 508 |
| HSSTROL3_node_13 | 509 |
| HSSTROL3_node_15 | 510 |
| HSSTROL3_node_19 | 511 |
| HSSTROL3_node_21 | 512 |
| HSSTROL3_node_24 | 513 |
| HSSTROL3_node_25 | 514 |
| HSSTROL3_node_26 | 515 |
| HSSTROL3_node_28 | 516 |
| HSSTROL3_node_29 | 517 |
| HSSTROL3_node_11 | 518 |
| HSSTROL3_node_17 | 519 |
| HSSTROL3_node_18 | 520 |
| HSSTROL3_node_20 | 521 |
| HSSTROL3_node_27 | 522 |

TABLE 3
Proteins of interest

| Protein Name | SEQ ID NO: | Corresponding Transcript(s) |
|---|---|---|
| HSSTROL3_P4 | 524 | HSSTROL3_T5 (SEQ ID NO: 85) |
| HSSTROL3_P5 | 525 | HSSTROL3_T8 (SEQ ID NO: 86); |
| | | HSSTROL3_T9 (SEQ ID NO: 87) |
| HSSTROL3_P7 | 526 | HSSTROL3_T10 (SEQ ID NO: 88) |
| HSSTROL3_P8 | 527 | HSSTROL3_T11 (SEQ ID NO: 505) |
| HSSTROL3_P9 | 528 | HSSTROL3_T12 (SEQ ID NO: 506) |

These sequences are variants of the known protein Stromelysin-3 precursor (SwissProt accession identifier MM11_HUMAN; known also according to the synonyms EC 3.4.24.-; Matrix metalloproteinase-11; MMP-11; ST3; SL-3), SEQ ID NO: 523, referred to herein as the previously known protein.

Protein Stromelysin-3 precursor (SEQ ID NO:523) is known or believed to have the following function(s): May play an important role in the progression of epithelial malignancies. The sequence for protein Stromelysin-3 precursor is given at the end of the application, as “Stromelysin-3 precursor amino acid sequence”.

The following GO Annotation(s) apply to the previously known protein. The following annotation(s) were found: proteolysis and peptidolysis; developmental processes; morphogenesis, which are annotation(s) related to Biological Process; stromelysin 3; calcium binding; zinc binding; hydrolase, which are annotation(s) related to Molecular Function; and extracellular matrix, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster HSSTROL3 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 75 and Table 4. This cluster is overexpressed (at least at a minimum level) in the following pathological conditions: transitional cell carcinoma, epithelial malignant tumors, a mixture of malignant tumors from different tissues and pancreas carinoma.

TABLE 4
Normal tissue distribution

| Name of Tissue | Number |
|---|---|
| adrenal | 0 |
| bladder | 0 |
| brain | 1 |
| colon | 63 |
| epithelial | 33 |
| general | 13 |
| head and neck | 101 |
| kidney | 0 |
| lung | 11 |
| breast | 8 |
| ovary | 14 |
| pancreas | 0 |
| prostate | 2 |
| skin | 99 |
| Thyroid | 0 |
| uterus | 181 |

TABLE 5
P values and ratios for expression in cancerous tissue

| Name of Tissue | P1 | P2 | SP1 | R3 | SP2 | R4 |
|---|---|---|---|---|---|---|
| adrenal | 1 | 4.6e−01 | 1 | 1.0 | 5.3e−01 | 1.9 |
| bladder | 2.7e−01 | 3.4e−01 | 3.3e−03 | 4.9 | 2.1e−02 | 3.3 |
| brain | 3.5e−01 | 2.6e−01 | 1 | 1.7 | 3.3e−01 | 2.8 |
| colon | 7.7e−02 | 1.5e−01 | 3.1e−01 | 1.4 | 5.2e−01 | 1.0 |
| epithelial | 1.2e−04 | 1.2e−02 | 1.3e−06 | 2.7 | 4.6e−02 | 1.4 |
| general | 5.4e−09 | 3.1e−05 | 1.8e−16 | 5.0 | 3.1e−07 | 2.6 |
| head and neck | 4.6e−01 | 4.3e−01 | 1 | 0.6 | 9.4e−01 | 0.7 |
| kidney | 2.5e−01 | 3.5e−01 | 1.1e−01 | 4.0 | 2.4e−01 | 2.8 |
| lung | 1.8e−01 | 4.5e−01 | 1.9e−01 | 2.7 | 5.1e−01 | 1.4 |
| breast | 2.0e−01 | 3.4e−01 | 7.3e−02 | 3.3 | 2.5e−01 | 2.0 |
| ovary | 2.6e−01 | 3.2e−01 | 2.2e−02 | 2.0 | 7.0e−02 | 1.6 |
| pancreas | 9.5e−02 | 1.8e−01 | 1.8e−04 | 7.8 | 1.6e−03 | 5.5 |
| prostate | 8.2e−01 | 7.8e−01 | 4.5e−01 | 1.8 | 5.6e−01 | 1.5 |
| skin | 5.2e−01 | 5.8e−01 | 7.1e−01 | 0.8 | 1 | 0.3 |
| Thyroid | 2.9e−01 | 2.9e−01 | 1 | 1.1 | 1 | 1.1 |
| uterus | 4.2e−01 | 8.0e−01 | 7.5e−01 | 0.6 | 9.9e−01 | 0.4 |

AS noted above, cluster HSSTROL3 features 6 transcript(s), which were listed in Table 1 above. These transcript(s) encode for protein(s) which are variant(s) of protein Stromelysin-3 precursor (SEQ ID NO:523). A description of each variant protein according to the present invention is now provided.

Variant protein HSSTROL3_P4 (SEQ ID NO:524) according to the present invention has an amino acid sequence as given at the end of the application; it is encoded by transcript(s) HSSTROL3_T5 (SEQ ID NO:85). An alignment is given to the known protein (Stromelysin-3 precursor (SEQ ID NO:523)) at the end of the application. One or more alignments to one or more previously published protein sequences are given at the end of the application. A brief description of the relationship of the variant protein according to the present invention to each such aligned protein is as follows:

Comparison Report Between HSSTROL3_P4 (SEQ ID NO:524) and MM11_HUMAN (SEQ ID NO:523):

1. An isolated chimeric polypeptide encoding for HSSTROL3_P4 (SEQ ID NO:524), comprising a first amino acid sequence being at least 90% homologous to MAPAAWLRSAAARALLPPMLLLLLQPPPLLARALPPDVHHLHAERRGPQPWHAALPSSPAPAPATQEAPR PASSLRPPRCGVPDPSDGLSARNRQKRFVLSGGRWEKTDLTYRILRFPWQLVQEQVRQTMAEALKVWSD VTPLTFTEVHEGRADIMIDFARYW corresponding to amino acids 1-163 of MM11_HUMAN (SEQ ID NO:523), which also corresponds to amino acids 1-163 of HSSTROL3_P4 (SEQ ID NO:524), a bridging amino acid H corresponding to amino acid 164 of HSSTROL3_P4 (SEQ ID NO:524), a second amino acid sequence being at least 90% homologous to GDDLPFDGPGGILAHAFFPKTHREGDVHFDYDETWTIGDDQGTDLLQVAAHEFGHVLGLQHTTAAKALM SAFYTFRYPLSLSPDDCRGVQHLYGQPWPTVTSRTPALGPQAGIDTNEIAPLEPDAPPDACEASFDAVSTIR GELFFFKAGFVWRLRGGQLQPGYPALASRHWQGLPSPVDAAFEDAQGHIWFFQGAQYWVYDGEKPVLG PAPLTELGLVRFPVHAALVWGPEKNKIYFFRGRDYWRFHPSTRRVDSPVPRRATDWRGVPSEIDAAFQDA DG corresponding to amino acids 165-445 of MM11_HUMAN (SEQ ID NO:523), which also corresponds to amino acids 165-445 of HSSTROL3_P4 (SEQ ID NO:524), and a third amino acid sequence being at least 70%, optionally at least 80%, preferably at least 85%, more preferably at least 90% and most preferably at least 95% homologous to a polypeptide having the sequence ALGVRQLVGGGHSSRFSHLVVAGLPHACHRKSGSSSQVLCPEPSALLSVAG (SEQ ID NO:1568) corresponding to amino acids 446-496 of HSSTROL3_P4 (SEQ ID NO:524), wherein said first amino acid sequence, bridging amino acid, second amino acid sequence and third amino acid sequence are contiguous and in a sequential order.

2. An isolated polypeptide encoding for a tail of HSSTROL3_P4 (SEQ ID NO:524), comprising a polypeptide being at least 70%, optionally at least about 80%, preferably at least about 85%, more preferably at least about 90% and most preferably at least about 95% homologous to the sequence ALGVRQLVGGGHSSRFSHLVVAGLPHACHRKSGSSSQVLCPEPSALLSVAG (SEQ ID NO:1568) in HSSTROL3_P4 (SEQ ID NO:524).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSSTROL3_P4 (SEQ ID NO:524) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein HSSTROL3_P4 (SEQ ID NO:524) sequence provides support for the deduced sequence of this variant protein according to the present invention).

TABLE 6
Amino acid mutations

| SNP position(s) on | Alternative | Previously known |
|---|---|---|
| amino acid sequence | amino acid(s) | SNP? |
| 38 | V -> A | Yes |
| 104 | R -> P | Yes |
| 214 | A -> | No |
| 323 | Q -> H | Yes |

Variant protein HSSTROL3_P4 (SEQ ID NO:524) is encoded by the following transcript(s): HSSTROL3_T5 (SEQ ID NO:85), for which the sequence(s) is/are given at the end of the application. The coding portion of transcript HSSTROL3_T5 (SEQ ID NO:85) is shown in bold; this coding portion starts at position 24 and ends at position 1511. The transcript also has the following SNPs as listed in Table 7 (given according to their position on the nucleotide sequence, with the alternative nucleic acid listed; the last column indicates whether the SNP is known or not; the presence of known SNPs in variant protein HSSTROL3_P4 (SEQ ID NO:524) sequence provides support for the deduced sequence of this variant protein according to the present invention).

TABLE 7
Nucleic acid SNPs

| SNP position on nucleotide | Alternative | Previously |
|---|---|---|
| sequence | nucleic acid | known SNP? |
| 136 | T -> C | Yes |
| 334 | G -> C | Yes |
| 663 | G -> | No |
| 699 | -> T | No |
| 992 | G -> C | Yes |
| 1528 | A -> G | Yes |
| 1710 | A -> G | Yes |
| 2251 | A -> G | Yes |
| 2392 | C -> | No |
| 2444 | C -> A | Yes |
| 2470 | A -> T | Yes |
| 2687 | -> G | No |
| 2696 | -> G | No |
| 2710 | C -> | No |
| 2729 | -> A | No |
| 2755 | T -> C | No |
| 2813 | A -> | No |
| 2813 | A -> C | No |
| 2963 | A -> | No |
| 2963 | A -> C | No |
| 2993 | T -> C | Yes |
| 3140 | -> T | No |

Variant protein HSSTROL3_P5 (SEQ ID NO:525) according to the present invention has an amino acid sequence as given at the end of the application; it is encoded by transcript(s) HSSTROL3_T8 (SEQ ID NO:86) and HSSTROL3_T9 (SEQ ID NO:87). An alignment is given to the known protein (Stromelysin-3 precursor (SEQ ID NO:523)) at the end of the application. One or more alignments to one or more previously published protein sequences are given at the end of the application. A brief description of the relationship of the variant protein according to the present invention to each such aligned protein is as follows:

Comparison Report Between HSSTROL3_P5 (SEQ ID NO:525) and MM11_HUMAN (SEQ ID NO:523):

1. An isolated chimeric polypeptide encoding for HSSTROL3_P5 (SEQ ID NO:525), comprising a first amino acid sequence being at least 90% homologous to MAPAAWLRSAAARALLPPMLLLLLQPPPLLARALPPDVHHLHAERRGPQPWHAALPSSPAPAPATQEAPR PASSLRPPRCGVPDPSDGLSARNRQKRFVLSGGRWEKTDLTYRILRFPWQLVQEQVRQTMAEALKVWSD VTPLTFTEVHEGRADIMIDFARYW corresponding to amino acids 1-163 of MM11_HUMAN (SEQ ID NO:523), which also corresponds to amino acids 1-163 of HSSTROL3_P5 (SEQ ID NO:525), a bridging amino acid H corresponding to amino acid 164 of HSSTROL3_P5 (SEQ ID NO:525), a second amino acid sequence being at least 90% homologous to GDDLPFDGPGGILAHAFFPKTHREGDVHFDYDETWTIGDDQGTDLLQVAAHEFGHVLGLQHTTAAKALM SAFYTFRYPLSLSPDDCRGVQHLYGQPWPTVTSRTPALGPQAGIDTNEIAPLEPDAPPDACEASFDAVSTIR GELFFFKAGFVWRLRGGQLQPGYPALASRHWQGLPSPVDAAFEDAQGHIWFFQ corresponding to amino acids 165-358 of MM11_HUMAN (SEQ ID NO:523), which also corresponds to amino acids 165-358 of HSSTROL3_P5 (SEQ ID NO:525), and a third amino acid sequence being at least 70%, optionally at least 80%, preferably at least 85%, more preferably at least 90% and most preferably at least 95% homologous to a polypeptide having the sequence ELGFPSSTGRDESLEHCRCQGLHK (SEQ ID NO:1569) corresponding to amino acids 359-382 of HSSTROL3_P5 (SEQ ID NO:525), wherein said first amino acid sequence, bridging amino acid, second amino acid sequence and third amino acid sequence are contiguous and in a sequential order.

2. An isolated polypeptide encoding for a tail of HSSTROL3_P5 (SEQ ID NO:525), comprising a polypeptide being at least 70%, optionally at least about 80%, preferably at least about 85%, more preferably at least about 90% and most preferably at least about 95% homologous to the sequence ELGFPSSTGRDESLEHCRCQGLHK (SEQ ID NO:1569) in HSSTROL3_P5 (SEQ ID NO:525).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSSTROL3_P5 (SEQ ID NO:525) also has the following non-silent SNPs (Single Nucleotide Polymorphisms) as listed in Table 8, (given according to their position(s) on the amino acid sequence, with the alternative amino acid(s) listed; the last column indicates whether the SNP is known or not; the presence of known SNPs in variant protein HSSTROL3_P5 (SEQ ID NO:525) sequence provides support for the deduced sequence of this variant protein according to the present invention).

TABLE 8
Amino acid mutations

| SNP position(s) on amino acid | Alternative | Previously |
|---|---|---|
| sequence | amino acid(s) | known SNP? |
| 38 | V -> A | Yes |
| 104 | R -> P | Yes |
| 214 | A -> | No |
| 323 | Q -> H | Yes |

Variant protein HSSTROL3_P5 (SEQ ID NO:525) is encoded by the following transcript(s): HSSTROL3_T8 (SEQ ID NO:86) and HSSTROL3_T9 (SEQ ID NO:87), for which the sequence(s) is/are given at the end of the application.

The coding portion of transcript HSSTROL3_T8 (SEQ ID NO:86) is shown in bold; this coding portion starts at position 24 and ends at position 1169. The transcript also has the following SNPs as listed in Table 9 (given according to their position on the nucleotide sequence, with the alternative nucleic acid listed; the last column indicates whether the SNP is known or not; the presence of known SNPs in variant protein HSSTROL3_P5 (SEQ ID NO:525) sequence provides support for the deduced sequence of this variant protein according to the present invention).

TABLE 9
Nucleic acid SNPs

| SNP position on nucleotide | Alternative | Previously |
|---|---|---|
| sequence | nucleic acid | known SNP? |
| 136 | T -> C | Yes |
| 334 | G -> C | Yes |
| 663 | G -> | No |
| 699 | -> T | No |
| 992 | G -> C | Yes |
| 1903 | C -> | No |
| 1955 | C -> A | Yes |
| 1981 | A -> T | Yes |
| 2198 | -> G | No |
| 2207 | -> G | No |
| 2221 | C -> | No |
| 2240 | -> A | No |
| 2266 | T -> C | No |
| 2324 | A -> | No |
| 2324 | A -> C | No |
| 2474 | A -> | No |
| 2474 | A -> C | No |
| 2504 | T -> C | Yes |
| 2651 | -> T | No |

The coding portion of transcript HSSTROL3_T9 (SEQ ID NO:87) is shown in bold; this coding portion starts at position 24 and ends at position 1169. The transcript also has the following SNPs as listed in Table 10 (given according to their position on the nucleotide sequence, with the alternative nucleic acid listed; the last column indicates whether the SNP is known or not; the presence of known SNPs in variant protein HSSTROL3_P5 (SEQ ID NO:525) sequence provides support for the deduced sequence of this variant protein according to the present invention).

TABLE 10
Nucleic acid SNPs

| SNP position on nucleotide | | Previously |
|---|---|---|
| sequence | Alternative nucleic acid | known SNP? |
| 136 | T -> C | Yes |
| 334 | G -> C | Yes |
| 663 | G -> | No |
| 699 | -> T | No |
| 992 | G -> C | Yes |
| 1666 | A -> G | Yes |
| 1848 | A -> G | Yes |
| 2389 | A -> G | Yes |
| 2530 | C -> | No |
| 2582 | C -> A | Yes |
| 2608 | A -> T | Yes |
| 2825 | -> G | No |
| 2834 | -> G | No |
| 2848 | C -> | No |
| 2867 | -> A | No |
| 2893 | T -> C | No |
| 2951 | A -> | No |
| 2951 | A -> C | No |
| 3101 | A -> | No |
| 3101 | A -> C | No |
| 3131 | T -> C | Yes |
| 3278 | -> T | No |

Variant protein HSSTROL3_P7 (SEQ ID NO:526) according to the present invention has an amino acid sequence as given at the end of the application; it is encoded by transcript(s) HSSTROL3_T10 (SEQ ID NO:88). An alignment is given to the known protein (Stromelysin-3 precursor (SEQ ID NO:523)) at the end of the application. One or more alignments to one or more previously published protein sequences are given at the end of the application. A brief description of the relationship of the variant protein according to the present invention to each such aligned protein is as follows:

Comparison Report Between HSSTROL3_P7 (SEQ ID NO:526) and MM11_HUMAN (SEQ ID NO:523):

1. An isolated chimeric polypeptide encoding for HSSTROL3_P7 (SEQ ID NO:526), comprising a first amino acid sequence being at least 90% homologous to MAPAAWLRSAAARALLPPMLLLLLQPPPLLARALPPDVHHLHAERRGPQPWHAALPSSPAPAPATQEAPR PASSLRPPRCGVPDPSDGLSARNRQKRFVLSGGRWEKTDLTYRILRFPWQLVQEQVRQTMAEALKVWSD VTPLTFTEVHEGRADIMIDFARYW corresponding to amino acids 1-163 of MM11_HUMAN (SEQ ID NO:523), which also corresponds to amino acids 1-163 of HSSTROL3_P7 (SEQ ID NO:526), a bridging amino acid H corresponding to amino acid 164 of HSSTROL3_P7 (SEQ ID NO:526), a second amino acid sequence being at least 90% homologous to GDDLPFDGPGGILAHAFFPKTHREGDVHFDYDETWTIGDDQGTDLLQVAAHEFGHVLGLQHTTAAKALM SAFYTFRYPLSLSPDDCRGVQHLYGQPWPTVTSRTPALGPQAGIDTNEIAPLEPDAPPDACEASFDAVSTIR GELFFFKAGFVWRLRGGQLQPGYPALASRHWQGLPSPVDAAFEDAQGHIWFFQG corresponding to amino acids 165-359 of MM11_HUMAN (SEQ ID NO:523), which also corresponds to amino acids 165-359 of HSSTROL3_P7 (SEQ ID NO:526), and a third amino acid sequence being at least 70%, optionally at least 80%, preferably at least 85%, more preferably at least 90% and most preferably at least 95% homologous to a polypeptide having the sequence TTGVSTPAPGV (SEQ ID NO:1570) corresponding to amino acids 360-370 of HSSTROL3_P7 (SEQ ID NO:526), wherein said first amino acid sequence, bridging amino acid, second amino acid sequence and third amino acid sequence are contiguous and in a sequential order.

2. An isolated polypeptide encoding for a tail of HSSTROL3_P7 (SEQ ID NO:526), comprising a polypeptide being at least 70%, optionally at least about 80%, preferably at least about 85%, more preferably at least about 90% and most preferably at least about 95% homologous to the sequence TTGVSTPAPGV (SEQ ID NO:1570) in HSSTROL3_P7 (SEQ ID NO:526).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSSTROL3_P7 (SEQ ID NO:526) also has the following non-silent SNPs (Single Nucleotide Polymorphisms) as listed in Table 11, (given according to their position(s) on the amino acid sequence, with the alternative amino acid(s) listed; the last column indicates whether the SNP is known or not; the presence of known SNPs in variant protein HSSTROL3_P7 (SEQ ID NO:526) sequence provides support for the deduced sequence of this variant protein according to the present invention).

TABLE 11
Amino acid mutations

| SNP position(s) on amino acid | | Previously |
|---|---|---|
| sequence | Alternative amino acid(s) | known SNP? |
| 38 | V -> A | Yes |
| 104 | R -> P | Yes |
| 214 | A -> | No |
| 323 | Q -> H | Yes |

Variant protein HSSTROL3_P7 (SEQ ID NO:526) is encoded by the following transcript(s): HSSTROL3_T10 (SEQ ID NO:88), for which the sequence(s) is/are given at the end of the application. The coding portion of transcript HSSTROL3_T10 (SEQ ID NO:88) is shown in bold; this coding portion starts at position 24 and ends at position 1133. The transcript also has the following SNPs as listed in Table 12 (given according to their position on the nucleotide sequence, with the alternative nucleic acid listed; the last column indicates whether the SNP is known or not; the presence of known SNPs in variant protein HSSTROL3_P7 (SEQ ID NO:526) sequence provides support for the deduced sequence of this variant protein according to the present invention).

TABLE 12
Nucleic acid SNPs

| SNP position on nucleotide | | Previously |
|---|---|---|
| sequence | Alternative nucleic acid | known SNP? |
| 136 | T -> C | Yes |
| 334 | G -> C | Yes |
| 663 | G -> | No |
| 699 | -> T | No |
| 992 | G -> C | Yes |
| 1386 | A -> G | Yes |
| 1568 | A -> G | Yes |
| 2109 | A -> G | Yes |
| 2250 | C -> | No |
| 2302 | C -> A | Yes |
| 2328 | A -> T | Yes |
| 2545 | -> G | No |
| 2554 | -> G | No |
| 2568 | C -> | No |
| 2587 | -> A | No |
| 2613 | T -> C | No |
| 2671 | A -> | No |
| 2671 | A -> C | No |
| 2821 | A -> | No |
| 2821 | A -> C | No |
| 2851 | T -> C | Yes |
| 2998 | -> T | No |

Variant protein HSSTROL3_P8 (SEQ ID NO:527) according to the present invention has an amino acid sequence as given at the end of the application; it is encoded by transcript(s) HSSTROL3_T11 (SEQ ID NO:505). An alignment is given to the known protein (Stromelysin-3 precursor (SEQ ID NO:523)) at the end of the application. One or more alignments to one or more previously published protein sequences are given at the end of the application. A brief description of the relationship of the variant protein according to the present invention to each such aligned protein is as follows:

Comparison Report Between HSSTROL3_P8 (SEQ ID NO:527) and MM11_HUMAN (SEQ ID NO:523):

1. An isolated chimeric polypeptide encoding for HSSTROL3_P8 (SEQ ID NO:527), comprising a first amino acid sequence being at least 90% homologous to MAPAAWLRSAAARALLPPMLLLLLQPPPLLARALPPDVHHLHAERRGPQPWHAALPSSPAPAPATQEAPR PASSLRPPRCGVPDPSDGLSARNRQKRFVLSGGRWEKTDLTYRILRFPWQLVQEQVRQTMAEALKVWSD VTPLTFTEVHEGRADIMIDFARYW corresponding to amino acids 1-163 of MM11_HUMAN (SEQ ID NO:523), which also corresponds to amino acids 1-163 of HSSTROL3_P8 (SEQ ID NO:527), a bridging amino acid H corresponding to amino acid 164 of HSSTROL3_P8 (SEQ ID NO:527), a second amino acid sequence being at least 90% homologous to GDDLPFDGPGGILAHAFFPKTHREGDVHFDYDETWTIGDDQGTDLLQVAAHEFGHVLGLQHTTAAKALM SAFYTFRYPLSLSPDDCRGVQHLYGQPWPTVTSRTPALGPQAGIDTNEIAPLE corresponding to amino acids 165-286 of MM11_HUMAN (SEQ ID NO:523), which also corresponds to amino acids 165-286 of HSSTROL3_P8 (SEQ ID NO:527), and a third amino acid sequence being at least 70%, optionally at least 80%, preferably at least 85%, more preferably at least 90% and most preferably at least 95% homologous to a polypeptide having the sequence VRPCLPVPLLLCWPL (SEQ ID NO:1571) corresponding to amino acids 287-301 of HSSTROL3_P8 (SEQ ID NO:527), wherein said first amino acid sequence, bridging amino acid, second amino acid sequence and third amino acid sequence are contiguous and in a sequential order.

2. An isolated polypeptide encoding for a tail of HSSTROL3_P8 (SEQ ID NO:527), comprising a polypeptide being at least 70%, optionally at least about 80%, preferably at least about 85%, more preferably at least about 90% and most preferably at least about 95% homologous to the sequence VRPCLPVPLLLCWPL (SEQ ID NO:1571) in HSSTROL3_P8 (SEQ ID NO:527).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSSTROL3_P8 (SEQ ID NO:527) also has the following non-silent SNPs (Single Nucleotide Polymorphisms) as listed in Table 13, (given according to their position(s) on the amino acid sequence, with the alternative amino acid(s) listed; the last column indicates whether the SNP is known or not; the presence of known SNPs in variant protein HSSTROL3_P8 (SEQ ID NO:527) sequence provides support for the deduced sequence of this variant protein according to the present invention).

TABLE 13
Amino acid mutations

| SNP position(s) on amino acid | | Previously |
|---|---|---|
| sequence | Alternative amino acid(s) | known SNP? |
| 38 | V -> A | Yes |
| 104 | R -> P | Yes |
| 214 | A -> | No |

Variant protein HSSTROL3_P8 (SEQ ID NO:527) is encoded by the following transcript(s): HSSTROL3_T11 (SEQ ID NO:505), for which the sequence(s) is/are given at the end of the application. The coding portion of transcript HSSTROL3_T11 (SEQ ID NO:505) is shown in bold; this coding portion starts at position 24 and ends at position 926. The transcript also has the following SNPs as listed in Table 14 (given according to their position on the nucleotide sequence, with the alternative nucleic acid listed; the last column indicates whether the SNP is known or not; the presence of known SNPs in variant protein HSSTROL3_P8 (SEQ ID NO:527) sequence provides support for the deduced sequence of this variant protein according to the present invention).

TABLE 14
Nucleic acid SNPs

| SNP position on nucleotide | | Previously |
|---|---|---|
| sequence | Alternative nucleic acid | known SNP? |
| 136 | T -> C | Yes |
| 334 | G -> C | Yes |
| 663 | G -> | No |
| 699 | -> T | No |
| 935 | G -> A | Yes |
| 948 | G -> A | Yes |
| 1084 | G -> C | Yes |
| 1557 | C -> | No |
| 1609 | C -> A | Yes |
| 1635 | A -> T | Yes |
| 1852 | -> G | No |
| 1861 | -> G | No |
| 1875 | C -> | No |
| 1894 | -> A | No |
| 1920 | T -> C | No |
| 1978 | A -> | No |
| 1978 | A -> C | No |
| 2128 | A -> | No |
| 2128 | A -> C | No |
| 2158 | T -> C | Yes |
| 2305 | -> T | No |

Variant protein HSSTROL3_P9 (SEQ ID NO:528) according to the present invention has an amino acid sequence as given at the end of the application; it is encoded by transcript(s) HSSTROL3_T12 (SEQ ID NO:506). An alignment is given to the known protein (Stromelysin-3 precursor (SEQ ID NO:523)) at the end of the application. One or more alignments to one or more previously published protein sequences are given at the end of the application. A brief description of the relationship of the variant protein according to the present invention to each such aligned protein is as follows:

Comparison Report Between HSSTROL3_P9 (SEQ ID NO:528) and MM11_HUMAN (SEQ ID NO:523):

1. An isolated chimeric polypeptide encoding for HSSTROL3_P9 (SEQ ID NO:528), comprising a first amino acid sequence being at least 90% homologous to MAPAAWLRSAAARALLPPMLLLLLQPPPLLARALPPDVHHLHAERRGPQPWHAALPSSPAPAPATQEAPR PASSLRPPRCGVPDPSDGLSARNRQK corresponding to amino acids 1-96 of MM11_HUMAN (SEQ ID NO:523), which also corresponds to amino acids 1-96 of HSSTROL3_P9 (SEQ ID NO:528), a second amino acid sequence being at least 90% homologous to RILRFPWQLVQEQVRQTMAEALKVWSDVTPLTFTEVHEGRADIMIDFARYW corresponding to amino acids 113-163 of MM11_HUMAN (SEQ ID NO:523), which also corresponds to amino acids 97-147 of HSSTROL3_P9 (SEQ ID NO:528), a bridging amino acid H corresponding to amino acid 148 of HSSTROL3_P9 (SEQ ID NO:528), a third amino acid sequence being at least 90% homologous to GDDLPFDGPGGILAHAFFPKTHREGDVHFDYDETWTIGDDQGTDLLQVAAHEFGHVLGLQHTTAAKALM SAFYTFRYPLSLSPDDCRGVQHLYGQPWPTVTSRTPALGPQAGIDTNEIAPLEPDAPPDACEASFDAVSTIR GELFFFKAGFVWRLRGGQLQPGYPALASRHWQGLPSPVDAAFEDAQGHIWFFQG corresponding to amino acids 165-359 of MM11_HUMAN (SEQ ID NO:523), which also corresponds to amino acids 149-343 of HSSTROL3_P9 (SEQ ID NO:528), and a fourth amino acid sequence being at least 70%, optionally at least 80%, preferably at least 85%, more preferably at least 90% and most preferably at least 95% homologous to a polypeptide having the sequence TTGVSTPAPGV (SEQ ID NO:1570) corresponding to amino acids 344-354 of HSSTROL3_P9 (SEQ ID NO:528), wherein said first amino acid sequence, second amino acid sequence, bridging amino acid, third amino acid sequence and fourth amino acid sequence are contiguous and in a sequential order.

2. An isolated chimeric polypeptide encoding for an edge portion of HSSTROL3_P9 (SEQ ID NO:528), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise KR, having a structure as follows: a sequence starting from any of amino acid numbers 96−x to 96; and ending at any of amino acid numbers 97+((n−2)−x), in which x varies from 0 to n−2.

3. An isolated polypeptide encoding for a tail of HSSTROL3_P9 (SEQ ID NO:528), comprising a polypeptide being at least 70%, optionally at least about 80%, preferably at least about 85%, more preferably at least about 90% and most preferably at least about 95% homologous to the sequence TTGVSTPAPGV (SEQ ID NO:1570) in HSSTROL3_P9 (SEQ ID NO:528).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HSSTROL3_P9 (SEQ ID NO:528) also has the following non-silent SNPs (Single Nucleotide Polymorphisms) as listed in Table 15, (given according to their position(s) on the amino acid sequence, with the alternative amino acid(s) listed; the last column indicates whether the SNP is known or not; the presence of known SNPs in variant protein HSSTROL3_P9 (SEQ ID NO:528) sequence provides support for the deduced sequence of this variant protein according to the present invention).

TABLE 15
Amino acid mutations

| SNP position(s) on amino acid | | Previously |
|---|---|---|
| sequence | Alternative amino acid(s) | known SNP? |
| 38 | V -> A | Yes |
| 198 | A -> | No |
| 307 | Q -> H | Yes |

Variant protein HSSTROL3_P9 (SEQ ID NO:528) is encoded by the following transcript(s): HSSTROL3_T12 (SEQ ID NO:506), for which the sequence(s) is/are given at the end of the application. The coding portion of transcript HSSTROL3_T12 (SEQ ID NO:506) is shown in bold; this coding portion starts at position 24 and ends at position 1085. The transcript also has the following SNPs as listed in Table 16 (given according to their position on the nucleotide sequence, with the alternative nucleic acid listed; the last column indicates whether the SNP is known or not; the presence of known SNPs in variant protein HSSTROL3_P9 (SEQ ID NO:528) sequence provides support for the deduced sequence of this variant protein according to the present invention).

TABLE 16
Nucleic acid SNPs

| SNP position on nucleotide | | Previously |
|---|---|---|
| sequence | Alternative nucleic acid | known SNP? |
| 136 | T -> C | Yes |
| 615 | G -> | No |
| 651 | -> T | No |
| 944 | G -> C | Yes |
| 1275 | C -> | No |
| 1327 | C -> A | Yes |
| 1353 | A -> T | Yes |
| 1570 | -> G | No |
| 1579 | -> G | No |
| 1593 | C -> | No |
| 1612 | -> A | No |
| 1638 | T -> C | No |
| 1696 | A -> | No |
| 1696 | A -> C | No |
| 1846 | A -> | No |
| 1846 | A -> C | No |
| 1876 | T -> C | Yes |
| 2023 | -> T | No |

As noted above, cluster HSSTROL3 features 16 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HSSTROL3_node_{—}6 (SEQ ID NO:507) according to the present invention is supported by 14 libraries. The number of libraries was determined as previously described. This segment can be found in the following transcript(s): HSSTROL3_T5 (SEQ ID NO:85), HSSTROL3_T8 (SEQ ID NO:86), HSSTROL3_T9 (SEQ ID NO:87), HSSTROL3_T10 (SEQ ID NO:88), HSSTROL3_T11 (SEQ ID NO:505) and HSSTROL3_T12 (SEQ ID NO:506). Table 17 below describes the starting and ending position of this segment on each transcript.

TABLE 17
Segment location on transcripts

| | Segment | Segment |
|---|---|---|
| Transcript name | starting position | ending position |
| HSSTROL3_T5 (SEQ ID NO: 85) | 1 | 131 |
| HSSTROL3_T8 (SEQ ID NO: 86) | 1 | 131 |
| HSSTROL3_T9 (SEQ ID NO: 87) | 1 | 131 |
| HSSTROL3_T10 (SEQ ID NO: 88) | 1 | 131 |
| HSSTROL3_T11 (SEQ ID NO: 505) | 1 | 131 |
| HSSTROL3_T12 (SEQ ID NO: 506) | 1 | 131 |

Segment cluster HSSTROL3_node_{—}10 (SEQ ID NO:508) according to the present invention is supported by 21 libraries. The number of libraries was determined as previously described. This segment can be found in the following transcript(s): HSSTROL3_T5 (SEQ ID NO:85), HSSTROL3_T8 (SEQ ID NO:86), HSSTROL3_T9 (SEQ ID NO:87), HSSTROL3_T10 (SEQ ID NO:88), HSSTROL3_T11 (SEQ ID NO:505) and HSSTROL3_T12 (SEQ ID NO:506). Table 18 below describes the starting and ending position of this segment on each transcript.

TABLE 18
Segment location on transcripts

| | Segment | Segment |
|---|---|---|
| Transcript name | starting position | ending position |
| HSSTROL3_T5 (SEQ ID NO: 85) | 132 | 313 |
| HSSTROL3_T8 (SEQ ID NO: 86) | 132 | 313 |
| HSSTROL3_T9 (SEQ ID NO: 87) | 132 | 313 |
| HSSTROL3_T10 (SEQ ID NO: 88) | 132 | 313 |
| HSSTROL3_T11 (SEQ ID NO: 505) | 132 | 313 |
| HSSTROL3_T12 (SEQ ID NO: 506) | 132 | 313 |

Segment cluster HSSTROL3_node_{—}13 (SEQ ID NO:509) according to the present invention is supported by 36 libraries. The number of libraries was determined as previously described. This segment can be found in the following transcript(s): HSSTROL3_T5 (SEQ ID NO:85), HSSTROL3_T8 (SEQ ID NO:86), HSSTROL3_T9 (SEQ ID NO:87), HSSTROL3_T10 (SEQ ID NO:88), HSSTROL3_T11 (SEQ ID NO:505) and HSSTROL3_T12 (SEQ ID NO:506). Table 19 below describes the starting and ending position of this segment on each transcript.

TABLE 19
Segment location on transcripts

| | Segment | Segment |
|---|---|---|
| Transcript name | starting position | ending position |
| HSSTROL3_T5 (SEQ ID NO: 85) | 362 | 505 |
| HSSTROL3_T8 (SEQ ID NO: 86) | 362 | 505 |
| HSSTROL3_T9 (SEQ ID NO: 87) | 362 | 505 |
| HSSTROL3_T10 (SEQ ID NO: 88) | 362 | 505 |
| HSSTROL3_T11 (SEQ ID NO: 505) | 362 | 505 |
| HSSTROL3_T12 (SEQ ID NO: 506) | 314 | 457 |

Segment cluster HSSTROL3_node_{—}15 (SEQ ID NO:510) according to the present invention is supported by 47 libraries. The number of libraries was determined as previously described. This segment can be found in the following transcript(s): HSSTROL3_T5 (SEQ ID NO:85), HSSTROL3_T8 (SEQ ID NO:86), HSSTROL3_T9 (SEQ ID NO:87), HSSTROL3_T10 (SEQ ID NO:88), HSSTROL3_T11 (SEQ ID NO:505) and HSSTROL3_T12 (SEQ ID NO:506). Table 20 below describes the starting and ending position of this segment on each transcript.

TABLE 20
Segment location on transcripts

| | Segment | Segment |
|---|---|---|
| Transcript name | starting position | ending position |
| HSSTROL3_T5 (SEQ ID NO: 85) | 506 | 639 |
| HSSTROL3_T8 (SEQ ID NO: 86) | 506 | 639 |
| HSSTROL3_T9 (SEQ ID NO: 87) | 506 | 639 |
| HSSTROL3_T10 (SEQ ID NO: 88) | 506 | 639 |
| HSSTROL3_T11 (SEQ ID NO: 505) | 506 | 639 |
| HSSTROL3_T12 (SEQ ID NO: 506) | 458 | 591 |

Segment cluster HSSTROL3_node_{—}19 (SEQ ID NO:511) according to the present invention is supported by 63 libraries. The number of libraries was determined as previously described. This segment can be found in the following transcript(s): HSSTROL3_T5 (SEQ ID NO:85), HSSTROL3_T8 (SEQ ID NO:86), HSSTROL3_T9 (SEQ ID NO:87), HSSTROL3_T10 (SEQ ID NO:88), HSSTROL3_T11 (SEQ ID NO:505) and HSSTROL3_T12 (SEQ ID NO:506). Table 21 below describes the starting and ending position of this segment on each transcript.

TABLE 21
Segment location on transcripts

| | Segment | Segment |
|---|---|---|
| Transcript name | starting position | ending position |
| HSSTROL3_T5 (SEQ ID NO: 85) | 699 | 881 |
| HSSTROL3_T8 (SEQ ID NO: 86) | 699 | 881 |
| HSSTROL3_T9 (SEQ ID NO: 87) | 699 | 881 |
| HSSTROL3_T10 (SEQ ID NO: 88) | 699 | 881 |
| HSSTROL3_T11 (SEQ ID NO: 505) | 699 | 881 |
| HSSTROL3_T12 (SEQ ID NO: 506) | 651 | 833 |

Segment cluster HSSTROL3_node_{—}21 (SEQ ID NO:512) according to the present invention is supported by 61 libraries. The number of libraries was determined as previously described. This segment can be found in the following transcript(s): HSSTROL3_T5 (SEQ ID NO:85), HSSTROL3_T8 (SEQ ID NO:86), HSSTROL3_T9 (SEQ ID NO:87), HSSTROL3_T10 (SEQ ID NO:88), HSSTROL3_T11 (SEQ ID NO:505) and HSSTROL3_T12 (SEQ ID NO:506). Table 22 below describes the starting and ending position of this segment on each transcript.

TABLE 22
Segment location on transcripts

| | Segment | Segment |
|---|---|---|
| Transcript name | starting position | ending position |
| HSSTROL3_T5 (SEQ ID NO: 85) | 882 | 1098 |
| HSSTROL3_T8 (SEQ ID NO: 86) | 882 | 1098 |
| HSSTROL3_T9 (SEQ ID NO: 87) | 882 | 1098 |
| HSSTROL3_T10 (SEQ ID NO: 88) | 882 | 1098 |
| HSSTROL3_T11 (SEQ ID NO: 505) | 974 | 1190 |
| HSSTROL3_T12 (SEQ ID NO: 506) | 834 | 1050 |

Segment cluster HSSTROL3_node_{—}24 (SEQ ID NO:513) according to the present invention is supported by 7 libraries. The number of libraries was determined as previously described. This segment can be found in the following transcript(s): HSSTROL3_T8 (SEQ ID NO:86) and HSSTROL3_T9 (SEQ ID NO:87). Table 23 below describes the starting and ending position of this segment on each transcript.

TABLE 23
Segment location on transcripts

| | Segment | Segment |
|---|---|---|
| Transcript name | starting position | ending position |
| HSSTROL3_T8 (SEQ ID NO: 86) | 1099 | 1236 |
| HSSTROL3_T9 (SEQ ID NO: 87) | 1099 | 1236 |

Segment cluster HSSTROL3_node_{—}25 (SEQ ID NO:514) according to the present invention is supported by 13 libraries. The number of libraries was determined as previously described. This segment can be found in the following transcript(s): HSSTROL3_T8 (SEQ ID NO:86). Table 24 below describes the starting and ending position of this segment on each transcript.

TABLE 24
Segment location on transcripts

| | Segment | Segment |
|---|---|---|
| Transcript name | starting position | ending position |
| HSSTROL3_T8 (SEQ ID NO: 86) | 1237 | 1536 |

Segment cluster HSSTROL3_node_{—}26 (SEQ ID NO: 515) according to the present invention is supported by 55 libraries. The number of libraries was determined as previously described. This segment can be found in the following transcript(s): HSSTROL3_T5 (SEQ ID NO:85), HSSTROL3_T8 (SEQ ID NO:86), HSSTROL3_T9 (SEQ ID NO:87) and HSSTROL3_T11 (SEQ ID NO:505). Table 25 below describes the starting and ending position of this segment on each transcript.

TABLE 25
Segment location on transcripts

| | Segment | Segment |
|---|---|---|
| Transcript name | starting position | ending position |
| HSSTROL3_T5 (SEQ ID NO: 85) | 1099 | 1240 |
| HSSTROL3_T8 (SEQ ID NO: 86) | 1537 | 1678 |
| HSSTROL3_T9 (SEQ ID NO: 87) | 1237 | 1378 |
| HSSTROL3_T11 (SEQ ID NO: 505) | 1191 | 1332 |

Segment cluster HSSTROL3_node_{—}28 (SEQ ID NO:516) according to the present invention is supported by 10 libraries. The number of libraries was determined as previously described. This segment can be found in the following transcript(s): HSSTROL3_T5 (SEQ ID NO:85), HSSTROL3_T9 (SEQ ID NO:87) and HSSTROL3_T10 (SEQ ID NO:88). Table 26 below describes the starting and ending position of this segment on each transcript.

TABLE 26
Segment location on transcripts

| | Segment | Segment |
|---|---|---|
| Transcript name | starting position | ending position |
| HSSTROL3_T5 (SEQ ID NO: 85) | 1357 | 2283 |
| HSSTROL3_T9 (SEQ ID NO: 87) | 1495 | 2421 |
| HSSTROL3_T10 (SEQ ID NO: 88) | 1215 | 2141 |

Segment cluster HSSTROL3_node_{—}29 (SEQ ID NO:517) according to the present invention is supported by 109 libraries. The number of libraries was determined as previously described. This segment can be found in the following transcript(s): HSSTROL3_T5 (SEQ ID NO:85), HSSTROL3_T8 (SEQ ID NO:86), HSSTROL3_T9 (SEQ ID NO:87), HSSTROL3_T10 (SEQ ID NO:88), HSSTROL3_T11 (SEQ ID NO:505) and HSSTROL3_T12 (SEQ ID NO:506). Table 27 below describes the starting and ending position of this segment on each transcript.

TABLE 27
Segment location on transcripts

| | Segment | Segment |
|---|---|---|
| Transcript name | starting position | ending position |
| HSSTROL3_T5 (SEQ ID NO: 85) | 2284 | 3194 |
| HSSTROL3_T8 (SEQ ID NO: 86) | 1795 | 2705 |
| HSSTROL3_T9 (SEQ ID NO: 87) | 2422 | 3332 |
| HSSTROL3_T10 (SEQ ID NO: 88) | 2142 | 3052 |
| HSSTROL3_T11 (SEQ ID NO: 505) | 1449 | 2359 |
| HSSTROL3_T12 (SEQ ID NO: 506) | 1167 | 2077 |

According to an optional embodiment of the present invention, short segments related to the above cluster are also provided. These segments are up to about 120 bp in length, and so are included in a separate description.

Segment cluster HSSTROL3_node_{—}11 (SEQ ID NO:518) according to the present invention is supported by 25 libraries. The number of libraries was determined as previously described. This segment can be found in the following transcript(s): HSSTROL3_T5 (SEQ ID NO:85), HSSTROL3_T8 (SEQ ID NO:86), HSSTROL3_T9 (SEQ ID NO:87), HSSTROL3_T10 (SEQ ID NO:88) and HSSTROL3_T11 (SEQ ID NO:505). Table 28 below describes the starting and ending position of this segment on each transcript.

TABLE 28
Segment location on transcripts

| | Segment | Segment |
|---|---|---|
| Transcript name | starting position | ending position |
| HSSTROL3_T5 (SEQ ID NO: 85) | 314 | 361 |
| HSSTROL3_T8 (SEQ ID NO: 86) | 314 | 361 |
| HSSTROL3_T9 (SEQ ID NO: 87) | 314 | 361 |
| HSSTROL3_T10 (SEQ ID NO: 88) | 314 | 361 |
| HSSTROL3_T11 (SEQ ID NO: 505) | 314 | 361 |

Segment cluster HSSTROL3_node_{—}17 (SEQ ID NO:519) according to the present invention is supported by 45 libraries. The number of libraries was determined as previously described. This segment can be found in the following transcript(s): HSSTROL3_T5 (SEQ ID NO:85), HSSTROL3_T8 (SEQ ID NO:86), HSSTROL3_T9 (SEQ ID NO:87), HSSTROL3_T10 (SEQ ID NO:88), HSSTROL3_T11 (SEQ ID NO:505) and HSSTROL3_T12 (SEQ ID NO:506). Table 29 below describes the starting and ending position of this segment on each transcript.

TABLE 29
Segment location on transcripts

| | Segment | Segment |
|---|---|---|
| Transcript name | starting position | ending position |
| HSSTROL3_T5 (SEQ ID NO: 85) | 640 | 680 |
| HSSTROL3_T8 (SEQ ID NO: 86) | 640 | 680 |
| HSSTROL3_T9 (SEQ ID NO: 87) | 640 | 680 |
| HSSTROL3_T10 (SEQ ID NO: 88) | 640 | 680 |
| HSSTROL3_T11 (SEQ ID NO: 505) | 640 | 680 |
| HSSTROL3_T12 (SEQ ID NO: 506) | 592 | 632 |

Segment cluster HSSTROL3_node_{—}18 (SEQ ID NO:520) according to the present invention can be found in the following transcript(s): HSSTROL3_T5 (SEQ ID NO:85), HSSTROL3_T8 (SEQ ID NO:86), HSSTROL3_T9 (SEQ ID NO:87), HSSTROL3_T10 (SEQ ID NO:88), HSSTROL3_T11 (SEQ ID NO:505) and HSSTROL3_T12 (SEQ ID NO:506). Table 30 below describes the starting and ending position of this segment on each transcript.

TABLE 30
Segment location on transcripts

| | Segment | Segment |
|---|---|---|
| Transcript name | starting position | ending position |
| HSSTROL3_T5 (SEQ ID NO: 85) | 681 | 698 |
| HSSTROL3_T8 (SEQ ID NO: 86) | 681 | 698 |
| HSSTROL3_T9 (SEQ ID NO: 87) | 681 | 698 |
| HSSTROL3_T10 (SEQ ID NO: 88) | 681 | 698 |
| HSSTROL3_T11 (SEQ ID NO: 505) | 681 | 698 |
| HSSTROL3_T12 (SEQ ID NO: 506) | 633 | 650 |

Segment cluster HSSTROL3_node_{—}20 (SEQ ID NO:521) according to the present invention is supported by 1 libraries. The number of libraries was determined as previously described. This segment can be found in the following transcript(s): HSSTROL3_T11 (SEQ ID NO:505). Table 31 below describes the starting and ending position of this segment on each transcript.

TABLE 31
Segment location on transcripts

| | Segment | Segment |
|---|---|---|
| Transcript name | starting position | ending position |
| HSSTROL3_T11 (SEQ ID NO: 505) | 882 | 973 |

Segment cluster HSSTROL3_node_{—}27 (SEQ ID NO:522) according to the present invention is supported by 50 libraries. The number of libraries was determined as previously described. This segment can be found in the following transcript(s): HSSTROL3_T5 (SEQ ID NO:85), HSSTROL3_T8 (SEQ ID NO:86), HSSTROL3_T9 (SEQ ID NO:87), HSSTROL3_T10 (SEQ ID NO:88), HSSTROL3_T11 (SEQ ID NO:505) and HSSTROL3_T12 (SEQ ID NO:506). Table 32 below describes the starting and ending position of this segment on each transcript.

TABLE 32
Segment location on transcripts

| | Segment | Segment |
|---|---|---|
| Transcript name | starting position | ending position |
| HSSTROL3_T5 (SEQ ID NO: 85) | 1241 | 1356 |
| HSSTROL3_T8 (SEQ ID NO: 86) | 1679 | 1794 |
| HSSTROL3_T9 (SEQ ID NO: 87) | 1379 | 1494 |
| HSSTROL3_T10 (SEQ ID NO: 88) | 1099 | 1214 |
| HSSTROL3_T11 (SEQ ID NO: 505) | 1333 | 1448 |
| HSSTROL3_T12 (SEQ ID NO: 506) | 1051 | 1166 |

### Variant Protein Alignment to the Previously Known Protein:

Sequence name: MM11_HUMAN (SEQ ID NO:523)
Sequence documentation:
Alignment of: HSSTROL3_P4 (SEQ ID NO:524) × M11_HUMAN (SEQ ID NO:523) ..
Alignment segment 1/1:

| | | | |
|---|---|---|---|
| Quality: | 4444.00 | Escore: | 0 |
| Matching length: | 445 | Total length: | 445 |
| Matching Percent Similarity: | 99.78 | Matching Percent Identity: | 99.78 |
| Total Percent Similarity: | 99.78 | Total Percent Identity: | 99.78 |
| Gaps: | 0 | | |
| Alignment: | | | |

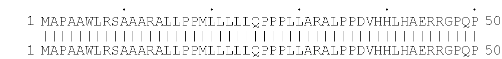

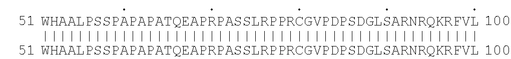

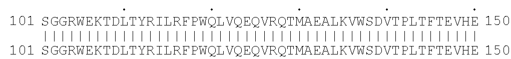

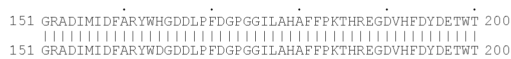

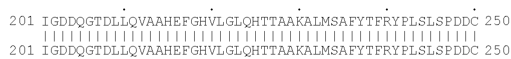

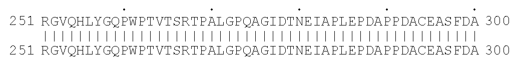

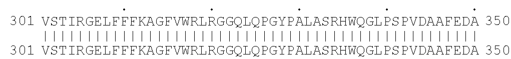

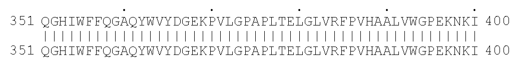

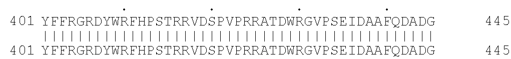

Sequence name: MM11_HUMAN (SEQ ID NO:523)
Sequence documentation:
Alignment of: HSSTROL3_P5 (SEQ ID NO:525) × MM11_HUMAN (SEQ ID NO:523) ..
Alignment segment 1/1:

| | | | |
|---|---|---|---|
| Quality: | 3566.00 | Escore: | 0 |
| Matching length: | 358 | Total length: | 358 |
| Matching Percent Similarity: | 99.72 | Matching Percent Identity: | 99.72 |
| Total Percent Similarity: | 99.72 | Total Percent Identity: | 99.72 |
| Gaps: | 0 | | |
| Alignment: | | | |

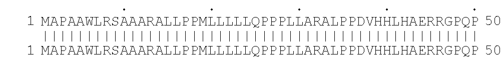

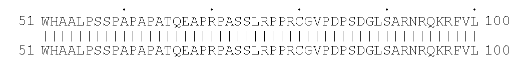

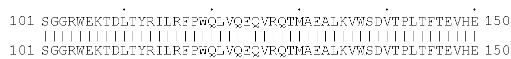

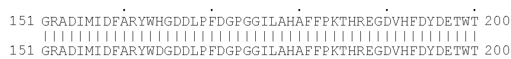

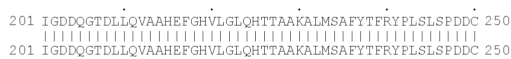

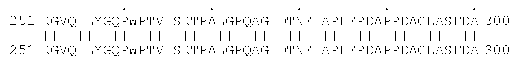

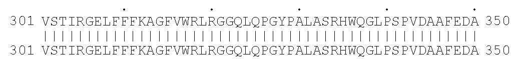

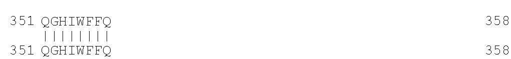

Sequence name: MM11_HUMAN (SEQ ID NO:523)
Sequence documentation:
Alignment of: HSSTROL3_P7 (SEQ ID NO:526) × MM11_HUMAN (SEQ ID NO:523) ..
Alignment segment 1/1:

| | | | |
|---|---|---|---|
| Quality: | 3575.00 | Escore: | 0 |
| Matching length: | 359 | Total length: | 359 |
| Matching Percent Similarity: | 99.72 | Matching Percent Identity: | 99.72 |
| Total Percent Similarity: | 99.72 | Total Percent Identity: | 99.72 |
| Gaps: | 0 | | |
| Alignment: | | | |

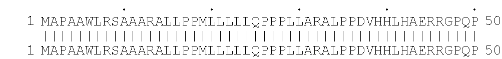

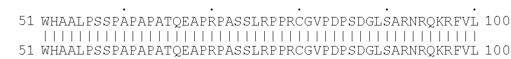

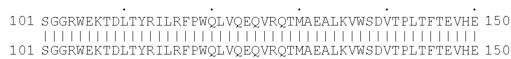

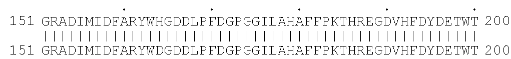

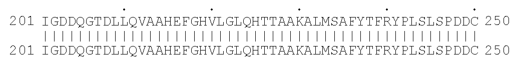

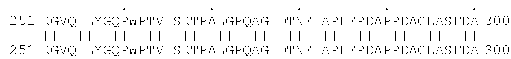

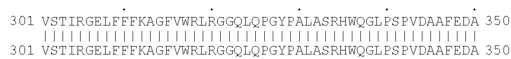

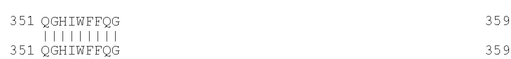

Sequence name: MM11_HUMAN (SEQ ID NO:523)
Sequence documentation:
Alignment of: HSSTROL3_P8 (SEQ ID NO:527) × MM11_HUMAN (SEQ ID NO:523) ..
Alignment segment 1/1:

| | | | |
|---|---|---|---|
| Quality: | 2838.00 | Escore: | 0 |
| Matching length: | 286 | Total length: | 286 |
| Matching Percent Similarity: | 99.65 | Matching Percent Identity: | 99.65 |
| Total Percent Similarity: | 99.65 | Total Percent Identity: | 99.65 |
| Gaps: | 0 | | |
| Alignment: | | | |

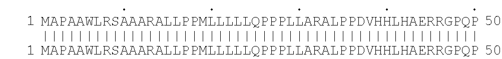

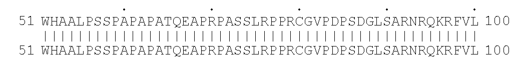

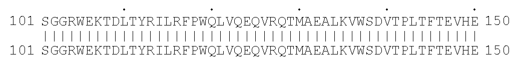

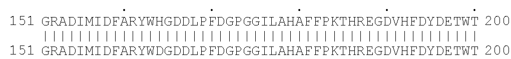

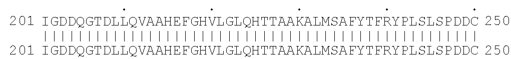

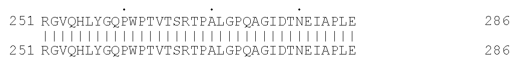

Sequence name: MM11_HUMAN (SEQ ID NO:523)
Sequence documentation:
Alignment of: HSSTROL3_P9 (SEQ ID NO:528) × MM11_HUMAN (SEQ ID NO:523) ..
Alignment segment 1/1:

| | | | |
|---|---|---|---|
| Quality: | 3316.00 | Escore: | 0 |
| Matching length: | 343 | Total length: | 359 |
| Matching Percent Similarity: | 99.71 | Matching Percent Identity: | 99.71 |
| Total Percent Similarity: | 95.26 | Total Percent Identity: | 95.26 |
| Gaps: | 1 | | |
| Alignment: | | | |

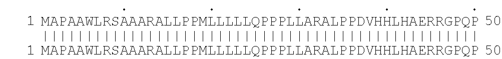

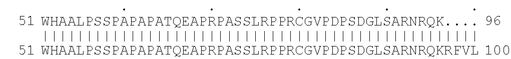

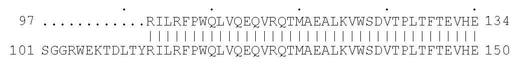

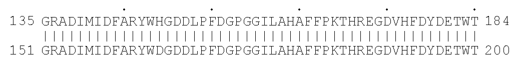

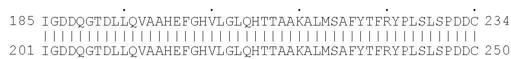

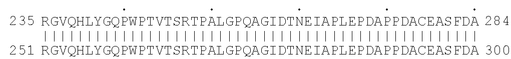

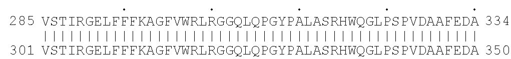

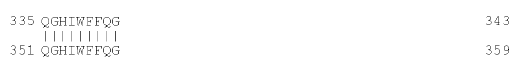

### Expression of Homo sapiens Matrix Metalloproteinase 11 (Stromelysin 3) (MMP11) HSSTROL3 Transcripts which are Detectable by Amplicon as Depicted in Sequence Name HSSTROL3 junc21-27 (SEQ ID NO:1312) in Normal and Cancerous Colon Tissues

Expression of Homo sapiens matrix metalloproteinase 11 (stromelysin 3) (MMP11) transcripts detectable by or according to junc21-27, HSSTROL3 junc21-27 amplicon (SEQ ID NO:1312) and primers HSSTROL3 junc21-27F (SEQ ID NO:1310) and HSSTROL3 junc21-27R (SEQ ID NO:1311) was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); HPRT1-amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

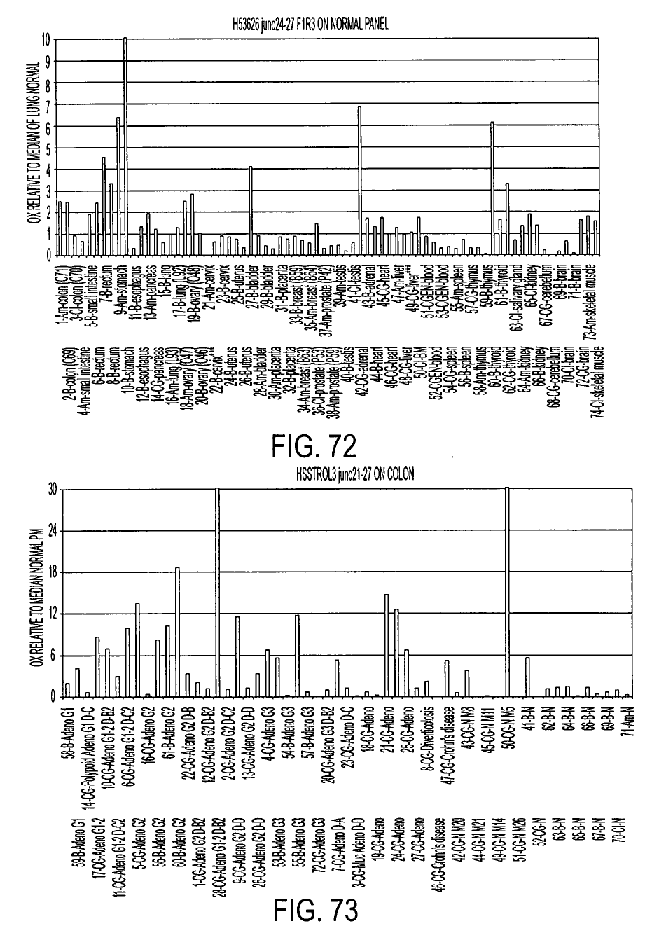

FIG. 73 is a histogram showing over expression of the above-indicated Homo sapiens matrix metalloproteinase 11 (stromelysin 3) (MMP11) transcripts in cancerous colon samples relative to the normal samples.

As is evident from FIG. 73, the expression of Homo sapiens matrix metalloproteinase 11 (stromelysin 3) (MMP11) transcripts detectable by the above amplicon(s) in cancer samples was higher than in the non-cancerous samples (Sample Nos. 41, 52, 62-67, 69-71 Table 1, “Tissue samples in testing panel”). Notably an over-expression of at least 6 fold was found in 14 out of 36 adenocarcinoma samples.

Primer pairs are also optionally and preferably encompassed within the present invention; for example, for the above experiment, the following primer pair was used as a non-limiting illustrative example only of a suitable primer pair: HSSTROL3 junc21-27F forward primer (SEQ ID NO:1310); and HSSTROL3 junc21-27R reverse primer (SEQ ID NO:1311).

The present invention also preferably encompasses any amplicon obtained through the use of any suitable primer pair; for example, for the above experiment, the following amplicon was obtained as a non-limiting illustrative example only of a suitable amplicon: HSSTROL3 junc21-27 (SEQ ID NO:1312).

Primers:
Forward primer HSSTROL3 junc21-27F (SEQ ID NO:
1310):
ACATTTGGTTCTTCCAAGGGACTAC
Reverse primer HSSTROL3 junc21-27R (SEQ ID NO:
1311):
TCGATCTCAGAGGGCACCC
Amplicon HSSTROL3 junc21-27 (SEQ ID NO:1312):
ACATTTGGTTCTTCCAAGGGACTACTGGCGTTTCCACCCCAGCACCCGGC
GTGTAGACAGTCCCGTGCCCCGCAGGGCCACTGACTGGAGAGGGGTGCCC
TCTGAGATCGA

### Expression of Homo sapiens Matrix Metalloproteinase 11 (Stromelysin 3) (MMP11) HSSTROL3 Transcripts which are Detectable by Amplicon as Depicted in Sequence Name HSSTROL3 Seg25 (SEQ ID NO: 1315) in Normal and Cancerous Colon Tissues

Expression of Homo sapiens matrix metalloproteinase 11 (stromelysin 3) (MMP11) transcripts detectable by or according to seg25, amplicon (SEQ ID NO: 1315) and primers HSSTROL3 seg25F (SEQ ID NO: 1313) and HSSTROL3 seg25R (SEQ ID NO: 1314) was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon —HPRT1-amplicon, SEQ ID NO:612), G6PD (GenBank Accession No. NM_{—}000402 (SEQ ID NO:1578); HPRT1-amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, “Tissue samples in testing panel”, above), to obtain a value of fold up-regulation for each sample relative to median of the normal PM samples.

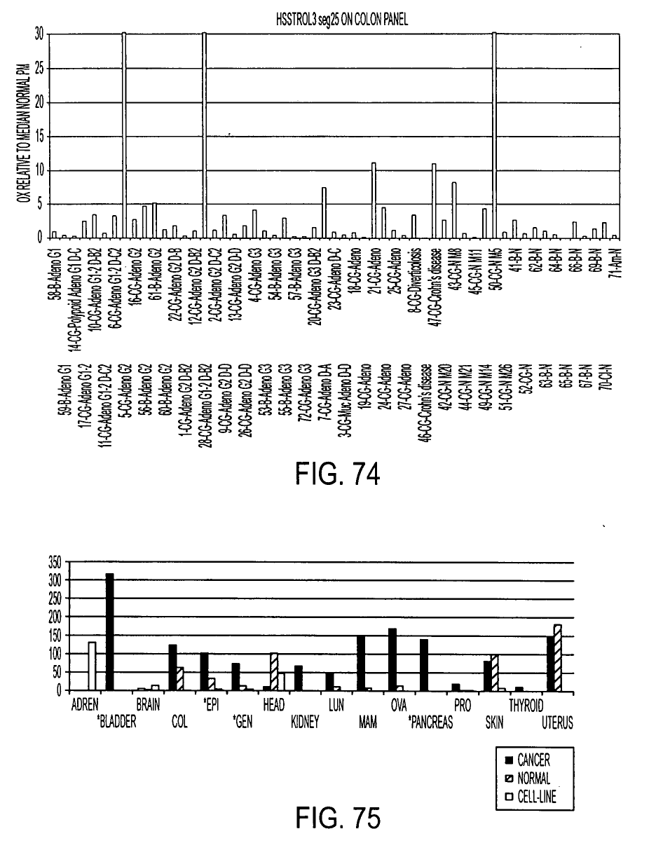

FIG. 74 is a histogram showing over expression of the above-indicated Homo sapiens matrix metalloproteinase 11 (stromelysin 3) (MMP11) transcripts in cancerous colon samples relative to the normal samples.

As is evident from FIG. 74, the expression of Homo sapiens matrix metalloproteinase 11 (stromelysin 3) (MMP11) transcripts detectable by the above amplicon(s) was higher in a few cancer samples than in the non-cancerous samples (Sample Nos. 41, 52, 62-67, 69-71 Table 1, “Tissue samples in testing panel”). Notably an over-expression of at least 5 fold was found in 5 out of 36 adenocarcinoma samples.

Primer pairs are also optionally and preferably encompassed within the present invention; for example, for the above experiment, the following primer pair was used as a non-limiting illustrative example only of a suitable primer pair: HSSTROL3 seg25F forward primer (SEQ ID NO: 1313); and HSSTROL3 seg25R reverse primer (SEQ ID NO: 1314).

The present invention also preferably encompasses any amplicon obtained through the use of any suitable primer pair; for example, for the above experiment, the following amplicon was obtained as a non-limiting illustrative example only of a suitable amplicon: HSSTROL3 seg25 (SEQ ID NO: 1315).

Primers:
Forward primer HSSTROL3 seg25F (SEQ ID NO: 1313):
CACTGCCCCAGCTTATGCC
Reverse primer HSSTROL3 seg25R (SEQ ID NO: 1314):
CTCTCCCAGCCTCAGTTTCCT
Amplicon HSSTROL3 seg25 (SEQ ID NO: 1315):
CACTGCCCCAGCTTATCCCAGGCCTCCCGCTTCCCTCTGCGGGTGGGGTG
CTGAGCAGGCATTATTGGCCTGCATGTTTTACTGATGAGGAAACTGAGGC
TGGGAGAG

### Expression of Homo sapiens Matrix Metalloproteinase 11 (Stromelysin 3) (MMP11) HSSTROL3 Transcripts which are Detectable by Amplicon as Depicted in Sequence Name HSSTROL3 Seg24 (SEQ ID NO: 1318) in Normal and Cancerous Colon Tissues

Expression of Homo sapiens matrix metalloproteinase 11 (stromelysin 3) (MMP11) transcripts detectable by or according to seg24, HSSTROL3 seg24 amplicon (SEQ ID NO: 1318) and primers HSSTROL3 seg24F (SEQ ID NO: 1316) and HSSTROL3 seg24R (SEQ ID NO: 1317) was measured by real time PCR. In parallel the expression of four housekeeping genes —PBGD (GenBank Accession No. BC019323 (SEQ ID NO:1576); amplicon —PBGD-amplicon, SEQ ID NO: 531), HPRT1 (GenBank Accession No. NM_{—}000194 (SEQ ID NO:1577); amplicon—HPRT1-amplicon, SEQ ID NO: 612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO: 1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold differential expression for each sample relative to median of the normal PM samples.

In one experiment that was carried out no differential expression in the cancerous samples relative to the normal PM samples was observed.

Primer pairs are also optionally and preferably encompassed within the present invention; for example, for the above experiment, the following primer pair was used as a non-limiting illustrative example only of a suitable primer pair: HSSTROL3 seg24F forward primer (SEQ ID NO: 1316); and HSSTROL3 seg24R reverse primer (SEQ ID NO: 1317).

The present invention also preferably encompasses any amplicon obtained through the use of any suitable primer pair; for example, for the above experiment, the following amplicon was obtained as a non-limiting illustrative example only of a suitable amplicon: HSSTROL3 seg24 (SEQ ID NO: 1318).

Primers:
Forward primer HSSTROL3 seg24F (SEQ ID NO: 1316):
ATTTCCATCCTCAACTGGCAGA
Reverse primer HSSTROL3 seg24R (SEQ ID NO: 1317):
TGCCCTGGAACCCACG
Amp1icon HSSTROL3 seg24 (SEQ ID NO: 1318):
ATTTCCATCCTCAACTGGCAGAGATGAGAGCCTGGAGCATTGCAGATGCC
AGGGACTTCACAAATGAAGGCACAGCATGGGAAACCTGCGTGGGTTCCAG
GGCA

### Expression of Stromelysin-3 Precursor (Matrix Metalloproteinase-11) (MMP-11) (ST3) (SL-3) HSSTROL3 Transcripts which are Detectable by Amplicon as Depicted in Sequence Name HSSTROL3 Seg24 (SEQ ID NO: 1318) in Different Normal Tissues

Expression of Stromelysin-3 precursor (EC 3.4.24.-) (Matrix metalloproteinase-11) (MMP-11) (ST3) (SL-3 transcripts detectable by or according to HSSTROL3 seg24 amplicon (SEQ ID NO: 1318) and HSSTROL3 seg24F (SEQ ID NO: 1316) and HSSTROL3 seg24R (SEQ ID NO: 1317) was measured by real time PCR. In parallel the expression of four housekeeping genes UBC (GenBank Accession No. BC000449 (SEQ ID NO:1582); amplicon—Ubiquitin-amplicon, SEQ ID NO: 1270) and SDHA (GenBank Accession No. NM_{—}004168 (SEQ ID NO:1583); amplicon—SDHA-amplicon, SEQ ID NO: 1273), RPL19 (GenBank Accession No. NM_{—}000981 (SEQ ID NO:1580); RPL19 amplicon, SEQ ID NO: 1264), TATA box (GenBank Accession No. NM_{—}003194 (SEQ ID NO:1581); TATA amplicon, SEQ ID NO: 1267) was measured similarly. For each RT sample, the expression of the above amplicon was normalized to the geometric mean of the quantities of the housekeeping genes. The normalized quantity of each RT sample was then divided by the median of the quantities of the lung samples (Sample Nos. 15-17 above), to obtain a value of relative expression of each sample relative to median of the lung samples.

Primers:
Forward primer HSSTROL3 seg24F (SEQ ID NO: 1316):
ATTTCCATCCTCAACTGGCAGA
Reverse primer HSSTROL3 seg24R (SEQ ID NO: 1317):
TGCCCTGGAACCCACG
Amplicon HSSTROL3 seg24 (SEQ ID NO: 1318):
ATTTCCATCCTCAACTGGCAGAGATGAGAGCCTGGAGCATTGCAGATGCC
AGGGACTTCACAAATGAAGGCACAGCATGGGAAACCTGCGTGGGTTCCAG
GGCA

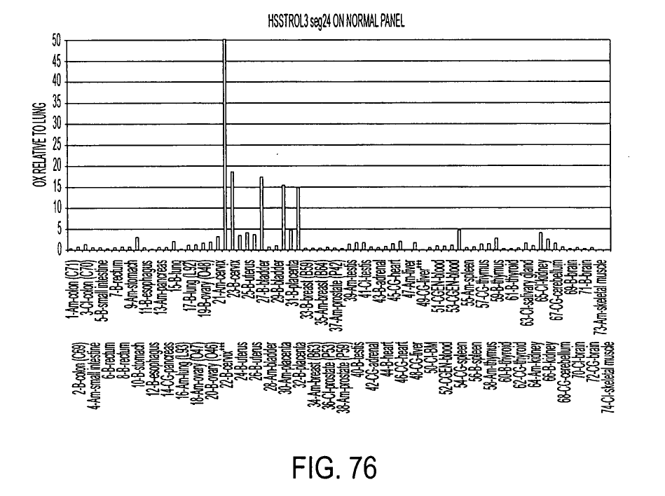

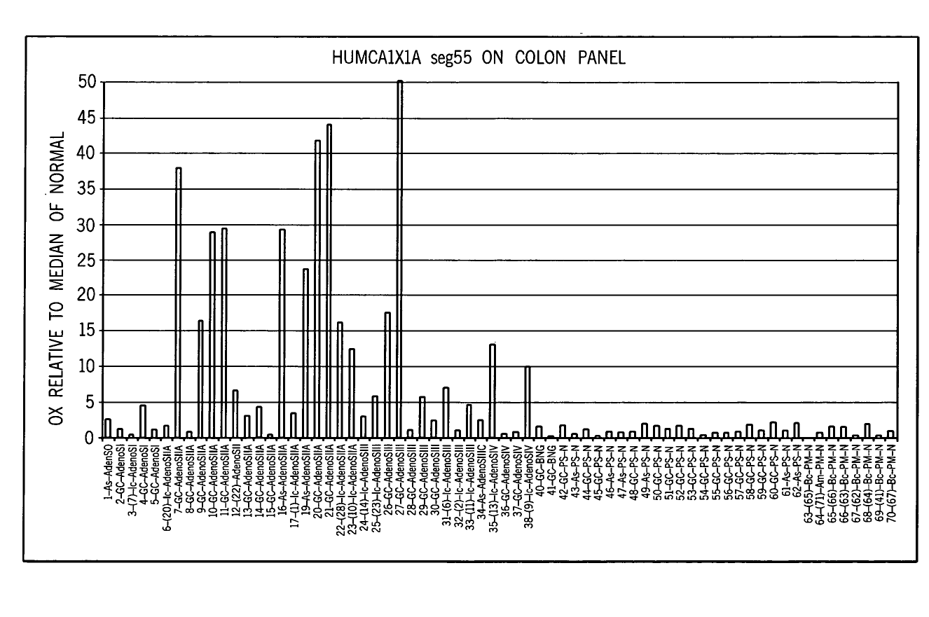

The results are presented in FIG. 76, showing the expression of Stromelysin-3 HSSTROL3 transcripts which are detectable by amplicon as depicted in sequence name HSSTROL3 seg24 in different normal tissues.

### Description for Cluster AA583399

Cluster AA583399 features 16 transcript(s) and 20 segment(s) of interest, the names for which are given in Tables 1 and 2, respectively, the sequences themselves are given at the end of the application. The selected protein variants are given in table 3.

TABLE 1
Transcripts of interest

| Transcript name | SEQ ID NO: |
|---|---|
| AA583399_PEA_1_T0 | 643 |
| AA583399_PEA_1_T1 | 644 |
| AA583399_PEA_1_T2 | 645 |
| AA583399_PEA_1_T3 | 646 |
| AA583399_PEA_1_T4 | 647 |
| AA583399_PEA_1_T5 | 648 |
| AA583399_PEA_1_T6 | 649 |
| AA583399_PEA_1_T7 | 650 |
| AA583399_PEA_1_T8 | 651 |
| AA583399_PEA_1_T9 | 652 |
| AA583399_PEA_1_T10 | 653 |
| AA583399_PEA_1_T11 | 654 |
| AA583399_PEA_1_T12 | 655 |
| AA583399_PEA_1_T15 | 656 |
| AA583399_PEA_1_T16 | 657 |
| AA583399_PEA_1_T17 | 658 |

TABLE 2
Segments of interest

| Segment Name | SEQ ID NO: |
|---|---|
| AA583399_PEA_1_node_0 | 659 |
| AA583399_PEA_1_node_3 | 660 |
| AA583399_PEA_1_node_9 | 661 |
| AA583399_PEA_1_node_10 | 662 |
| AA583399_PEA_1_node_12 | 663 |
| AA583399_PEA_1_node_14 | 664 |
| AA583399_PEA_1_node_21 | 665 |
| AA583399_PEA_1_node_24 | 666 |
| AA583399_PEA_1_node_25 | 667 |
| AA583399_PEA_1_node_29 | 668 |
| AA583399_PEA_1_node_1 | 669 |
| AA583399_PEA_1_node_2 | 670 |
| AA583399_PEA_1_node_4 | 671 |
| AA583399_PEA_1_node_5 | 672 |
| AA583399_PEA_1_node_6 | 673 |
| AA583399_PEA_1_node_7 | 674 |
| AA583399_PEA_1_node_8 | 675 |
| AA583399_PEA_1_node_11 | 676 |
| AA583399_PEA_1_node_19 | 677 |
| AA583399_PEA_1_node_27 | 678 |

TABLE 3
Proteins of interest

| Protein Name | SEQ ID NO: | Corresponding Transcript(s) |
|---|---|---|
| AA583399_PEA_1_P3 | 683 | AA583399_PEA_1_T1 (SEQ ID NO: 644); |
| | | AA583399_PEA_1_T6 (SEQ ID NO: 649); |
| | | AA583399_PEA_1_T9 (SEQ ID NO: 652) |
| AA583399_PEA_1_P2 | 684 | AA583399_PEA_1_T3 (SEQ ID NO: 646); |
| | | AA583399_PEA_1_T4 (SEQ ID NO: 647); |
| | | AA583399_PEA_1_T5 (SEQ ID NO: 648) |
| AA583399_PEA_1_P4 | 685 | AA583399_PEA_1_T7 (SEQ ID NO: 650) |
| AA583399_PEA_1_P5 | 686 | AA583399_PEA_1_T8 (SEQ ID NO: 651) |
| AA583399_PEA_1_P6 | 687 | AA583399_PEA_1_T12 (SEQ ID NO: 655); |
| | | AA583399_PEA_1_T16 (SEQ ID NO: 657) |
| AA583399_PEA_1_P8 | 688 | AA583399_PEA_1_T17 (SEQ ID NO: 658) |
| AA583399_PEA_1_P10 | 689 | AA583399_PEA_1_T0 (SEQ ID NO: 643) |
| AA583399_PEA_1_P11 | 690 | AA583399_PEA_1_T2 (SEQ ID NO: 645) |
| AA583399_PEA_1_P12 | 691 | AA583399_PEA_1_T10 (SEQ ID NO: 653); |
| | | AA583399_PEA_1_T11 (SEQ ID NO: 654) |
| AA583399_PEA_1_P14 | 692 | AA583399_PEA_1_T15 (SEQ ID NO: 656) |

These sequences are variants of the known protein Myeloma overexpressed gene protein (SwissProt accession identifier MYEO_HUMAN; known also according to the synonyms Oncogene in multiple myeloma), SEQ ID NO: 679, referred to herein as the previously known protein.

The sequence for protein Myeloma overexpressed gene protein (SEQ ID NO:679) is given at the end of the application, as “Myeloma overexpressed gene protein amino acid sequence”. Known polymorphisms for this sequence are as shown in Table 4.

TABLE 4
Amino acid mutations for Known Protein

| SNP position(s) | |
|---|---|
| on amino | |
| acid sequence | Comment |
| 159 | A -> V (in dbSNP: 7103126). /FTId = VAR_016603. |
| 198 | R -> Q |
| 219 | V -> M |
| 271 | G -> R |

Cluster AA583399 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

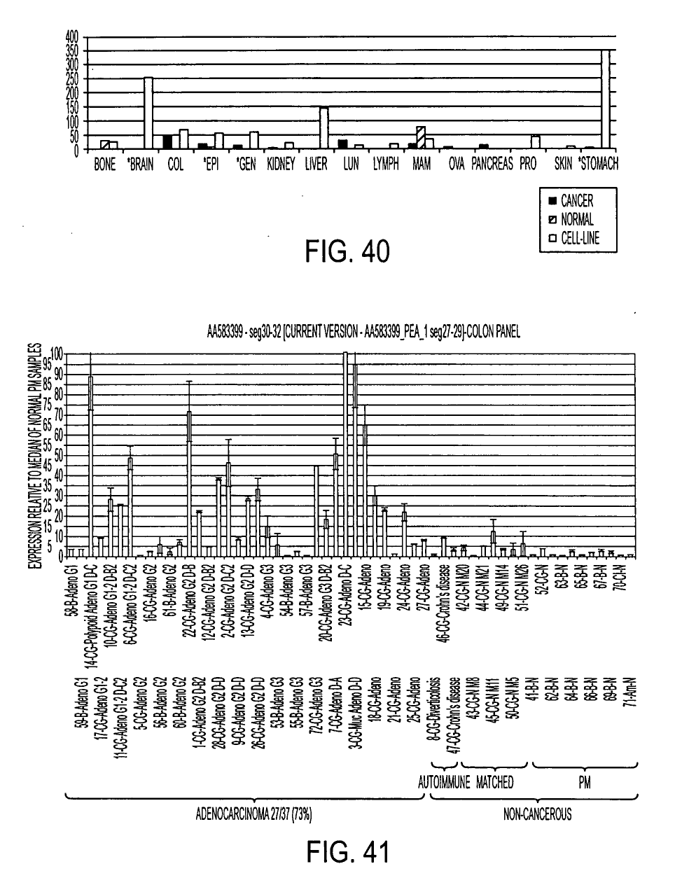

Overall, the following results were obtained as shown with regard to the histograms in FIG. 40 and Table 5. This cluster is overexpressed (at least at a minimum level) in the following pathological conditions: brain malignant tumors, epithelial malignant tumors, a mixture of malignant tumors from different tissues and gastric carcinoma.

TABLE 5
Normal tissue distribution

| Name of Tissue | Number |
|---|---|
| bone | 32 |
| brain | 0 |
| colon | 0 |
| epithelial | 4 |
| general | 2 |
| kidney | 0 |
| liver | 0 |
| lung | 2 |
| lymph nodes | 0 |
| breast | 79 |
| ovary | 0 |
| pancreas | 0 |
| prostate | 0 |
| skin | 0 |
| stomach | 0 |

TABLE 6
P values and ratios for expression in cancerous tissue

| Name of Tissue | P1 | P2 | SP1 | R3 | SP2 | R4 |
|---|---|---|---|---|---|---|
| bone | 9.2e−01 | 5.8e−01 | 1 | 0.5 | 9.1e−01 | 0.8 |
| brain | 9.8e−01 | 1.4e−01 | 1 | 0.8 | 2.0e−09 | 11.1 |
| colon | 1.6e−01 | 7.3e−02 | 3.4e−01 | 2.5 | 2.7e−01 | 2.7 |
| epithelial | 3.0e−03 | 1.3e−04 | 1.1e−01 | 2.3 | 5.4e−06 | 4.1 |
| general | 1.1e−05 | 3.2e−10 | 2.0e−03 | 4.5 | 1.1e−17 | 10.7 |
| kidney | 6.5e−01 | 5.1e−01 | 1 | 1.1 | 7.0e−01 | 1.5 |
| liver | 1 | 1.3e−01 | 1 | 1.0 | 1.6e−01 | 2.2 |
| lung | 1.7e−01 | 2.7e−01 | 1.7e−01 | 3.5 | 2.4e−01 | 2.5 |
| lymph nodes | 1 | 5.7e−01 | 1 | 1.0 | 5.8e−01 | 1.7 |
| breast | 8.8e−01 | 8.6e−01 | 9.0e−01 | 0.5 | 8.5e−01 | 0.6 |
| ovary | 1.6e−01 | 1.9e−01 | 1 | 1.1 | 1 | 1.1 |
| pancreas | 3.3e−01 | 4.4e−01 | 4.2e−01 | 2.4 | 5.3e−01 | 1.9 |
| prostate | 1 | 7.8e−01 | 1 | 1.0 | 5.6e−01 | 1.7 |
| skin | 1 | 4.4e−01 | 1 | 1.0 | 6.4e−01 | 1.6 |
| stomach | 3.6e−01 | 1.3e−01 | 1 | 1.1 | 1.8e−03 | 3.8 |

As noted above, cluster AA583399 features 16 transcript(s), which were listed in Table 1 above. These transcript(s) encode for protein(s) which are variant(s) of protein Myeloma overexpressed gene protein (SEQ ID NO:679). A description of each variant protein according to the present invention is now provided.

Variant protein AA583399_PEA_{—}1_P3 (SEQ ID NO:683) according to the present invention has an amino acid sequence as given at the end of the application; it is encoded by transcript(s) AA583399_PEA_{—}1_T1 (SEQ ID NO:644). The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide. Variant protein AA583399_PEA_{—}1_P3 (SEQ ID NO:683) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein AA583399_PEA_{—}1_P3 (SEQ ID NO:683) sequence provides support for the deduced sequence of this variant protein according to the present invention).

TABLE 7
Amino acid mutations

| SNP position(s) | | |
|---|---|---|
| on amino acid | Alternative | Previously |
| sequence | amino acid(s) | known SNP? |
| 159 | V -> A | Yes |
| 198 | R -> Q | Yes |
| 219 | M -> V | No |
| 271 | G -> R | Yes |
| 284 | P -> T | Yes |

Variant protein AA583399_PEA_{—}1_P3 (SEQ ID NO:683) is encoded by the following transcript(s): AA583399_PEA_{—}1_T1 (SEQ ID NO:644), for which the sequence(s) is/are given at the end of the application. The coding portion of transcript AA583399_PEA_{—}1_T1 (SEQ ID NO:644) is shown in bold; this coding portion starts at position 587 and ends at position 1525. The transcript also has the following SNPs as listed in Table 8 (given according to their position on the nucleotide sequence, with the alternative nucleic acid listed; the last column indicates whether the SNP is known or not; the presence of known SNPs in variant protein AA583399_PEA_{—}1_P3 (SEQ ID NO:683) sequence provides support for the deduced sequence of this variant protein according to the present invention).

TABLE 8
Nucleic acid SNPs

| SNP position | | |
|---|---|---|
| on nucleotide | Alternative | Previously |
| sequence | nucleic acid | known SNP? |
| 380 | A -> G | Yes |
| 805 | C -> T | Yes |
| 1062 | T -> C | Yes |
| 1179 | G -> A | Yes |
| 1241 | A -> G | No |
| 1397 | G -> C | Yes |
| 1436 | C -> A | Yes |
| 1653 | C -> T | No |
| 1657 | T -> G | No |

Variant protein AA583399_PEA_{—}1_P2 (SEQ ID NO:684) according to the present invention has an amino acid sequence as given at the end of the application; it is encoded by transcript(s) AA583399_PEA_{—}1_T3 (SEQ ID NO:646). An alignment is given to the known protein (Myeloma overexpressed gene protein (SEQ ID NO:679)) at the end of the application. One or more alignments to one or more previously published protein sequences are given at the end of the application. A brief description of the relationship of the variant protein according to the present invention to each such aligned protein is as follows:

Comparison Report Between AA583399_PEA_{—}1_P2 (SEQ ID NO:684) and MYEO_HUMAN_V1 (SEQ ID NO: 680):

1. An isolated chimeric polypeptide encoding for AA583399_PEA_{—}1_P2 (SEQ ID NO:684), comprising a first amino acid sequence being at least 90% homologous to MFTRQAGHFVEGSKAGRSRGRLCLSQALRVAVRGAFVSLWFAAGAGDRERNKGDKGAQTGAGLSQEAE DVDVSRARRVTDAPQGTLCGTGNRNSGSQSARVVGVAHLGEAFRVGVEQAISSCPEEVHGRHGLSMEIM WARMDVALRSPGRGLLAGAGALCMTLAESSCPDYERGRRACLTLHRHPTPHCSTWGLPLRVAGSWLTV VTVEALGGWRMGVRRTGQVGPTMHPPPVSGASPLLLHHLLLLLLIIILTC corresponding to amino acids 59-313 of MYEO_HUMAN_V1 (SEQ ID NO:680), which also corresponds to amino acids 1-255 of AA583399_PEA_{—}1_P2 (SEQ ID NO:684).

It should be noted that the known protein sequence (MYEO_HUMAN (SEQ ID NO:679)) has one or more changes than the sequence given at the end of the application and named as being the amino acid sequence for MYEO_HUMAN_V1 (SEQ ID NO:680). These changes were previously known to occur and are listed in the table below.

TABLE 9
Changes to MYEO_HUMAN_V1 (SEQ ID NO: 680)

| SNP position(s) on amino | |
|---|---|
| acid sequence | Type of change |
| 160 | variant |
| 220 | conflict |

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein AA583399_PEA_{—}1_P2 (SEQ ID NO:684) is encoded by the following transcript(s): AA583399_PEA_{—}1_T3 (SEQ ID NO:646), for which the sequence(s) is/are given at the end of the application. The coding portion of transcript AA583399_PEA_{—}1_T3 (SEQ ID NO:646) is shown in bold; this coding portion starts at position 689 and ends at position 1453. The transcript also has the following SNPs as listed in Table 10 (given according to their position on the nucleotide sequence, with the alternative nucleic acid listed; the last column indicates whether the SNP is known or not; the presence of known SNPs in variant protein AA583399_PEA_{—}1_P2 (SEQ ID NO:684) sequence provides support for the deduced sequence of this variant protein according to the present invention).

TABLE 10
Nucleic acid SNPs

| SNP position on | Alternative | Previously |
|---|---|---|
| nucleotide sequence | nucleic acid | known SNP? |
| 380 | A -> G | Yes |
| 733 | C -> T | Yes |
| 990 | T -> C | Yes |
| 1107 | G -> A | Yes |
| 1169 | A -> G | No |
| 1325 | G -> C | Yes |
| 1364 | C -> A | Yes |
| 1581 | C -> T | No |
| 1585 | T -> G | No |

Variant protein AA583399_PEA_{—}1_P4 (SEQ ID NO:685) according to the present invention has an amino acid sequence as given at the end of the application; it is encoded by transcript(s) AA583399_PEA_{—}1_T7 (SEQ ID NO:650). An alignment is given to the known protein (Myeloma overexpressed gene protein (SEQ ID NO:679)) at the end of the application. One or more alignments to one or more previously published protein sequences are given at the end of the application. A brief description of the relationship of the variant protein according to the present invention to each such aligned protein is as follows:

Comparison Report Between AA583399_PEA_{—}1_P4 (SEQ ID NO:685) and MYEO_HUMAN_V1 (SEQ ID NO:680):

1. An isolated chimeric polypeptide encoding for AA583399_PEA_{—}1_P4 (SEQ ID NO:685), comprising a first amino acid sequence being at least 70%, optionally at least 80%, preferably at least 85%, more preferably at least 90% and most preferably at least 95% homologous to a polypeptide having the sequence MSDLFIGFLVCSLSPLGTGTRCSCSPG (SEQ ID NO:1479) corresponding to amino acids 1-27 of AA583399_PEA_{—}1_P4 (SEQ ID NO:685), and a second amino acid sequence being at least 90% homologous to RNSGSQSARVVGVAHLGEAFRVGVEQAISSCPEEVHGRHGLSMEIMWARMDVALRSPGRGLLAGAGALC MTLAESSCPDYERGRRACLTLHRHPTPHCSTWGLPLRVAGSWLTVVTVEALGGWRMGVRRTGQVGPTM HPPPVSGASPLLLHHLLLLLLIIILTC corresponding to amino acids 150-313 of MYEO_HUMAN_V1 (SEQ ID NO:680), which also corresponds to amino acids 28-191 of AA583399_PEA_{—}1_P4 (SEQ ID NO:685), wherein said first amino acid sequence and second amino acid sequence are contiguous and in a sequential order.

2. An isolated polypeptide encoding for a head of AA583399_PEA_{—}1_P4 (SEQ ID NO:685), comprising a polypeptide being at least 70%, optionally at least about 80%, preferably at least about 85%, more preferably at least about 90% and most preferably at least about 95% homologous to the sequence MSDLFIGFLVCSLSPLGTGTRCSCSPG (SEQ ID NO:1479) of AA583399_PEA_{—}1_P4 (SEQ ID NO:685).

It should be noted that the known protein sequence (MYEO_HUMAN (SEQ ID NO:679)) has one or more changes than the sequence given at the end of the application and named as being the amino acid sequence for MYEO_HUMAN_V1 (SEQ ID NO:680). These changes were previously known to occur and are listed in the table below.

TABLE 11
Changes to MYEO_HUMAN_V1 (SEQ ID NO: 680)

| SNP position(s) on amino | |
|---|---|
| acid sequence | Type of change |
| 160 | variant |
| 220 | conflict |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide.

Variant protein AA583399_PEA_{—}1_P4 (SEQ ID NO:685) also has the following non-silent SNPs (Single Nucleotide Polymorphisms) as listed in Table 12, (given according to their position(s) on the amino acid sequence, with the alternative amino acid(s) listed; the last column indicates whether the SNP is known or not; the presence of known SNPs in variant protein AA583399_PEA_{—}1_P4 (SEQ ID NO:685) sequence provides support for the deduced sequence of this variant protein according to the present invention).

TABLE 12
Amino acid mutations

| SNP position(s) on | Alternative | Previously |
|---|---|---|
| amino acid sequence | amino acid(s) | known SNP? |
| 37 | V -> A | Yes |
| 76 | R -> Q | Yes |
| 97 | M -> V | No |
| 149 | G -> R | Yes |
| 162 | P -> T | Yes |

Variant protein AA583399_PEA_{—}1_P4 (SEQ ID NO:685) is encoded by the following transcript(s): AA583399_PEA_{—}1_T7 (SEQ ID NO:650), for which the sequence(s) is/are given at the end of the application. The coding portion of transcript AA583399_PEA_{—}1_T7 (SEQ ID NO:650) is shown in bold; this coding portion starts at position 789 and ends at position 1361. The transcript also has the following SNPs as listed in Table 13 (given according to their position on the nucleotide sequence, with the alternative nucleic acid listed; the last column indicates whether the SNP is known or not; the presence of known SNPs in variant protein AA583399_PEA_{—}1_P4 (SEQ ID NO:685) sequence provides support for the deduced sequence of this variant protein according to the present invention).

TABLE 13
Nucleic acid SNPs

| SNP position on | Alternative | Previously |
|---|---|---|
| nucleotide sequence | nucleic acid | known SNP? |
| 380 | A -> G | Yes |
| 898 | T -> C | Yes |
| 1015 | G -> A | Yes |
| 1077 | A -> G | No |
| 1233 | G -> C | Yes |
| 1272 | C -> A | Yes |
| 1489 | C -> T | No |
| 1493 | T -> G | No |

Variant protein AA583399_PEA_{—}1_P5 (SEQ ID NO:686) according to the present invention has an amino acid sequence as given at the end of the application; it is encoded by transcript(s) AA583399_PEA_{—}1_T8 (SEQ ID NO:651). An alignment is given to the known protein (Myeloma overexpressed gene protein (SEQ ID NO:679)) at the end of the application. One or more alignments to one or more previously published protein sequences are given at the end of the application. A brief description of the relationship of the variant protein according to the present invention to each such aligned protein is as follows:

Comparison Report Between AA583399_PEA_{—}1_P5 (SEQ ID NO:686) and MYEO_HUMAN_V2 (SEQ ID NO: 681):

1. An isolated chimeric polypeptide encoding for AA583399_PEA_{—}1_P5 (SEQ ID NO:686), comprising a first amino acid sequence being at least 90% homologous to MEIMWARMDVALRSPGRGLLAGAGALCMTLAESSCPDYERGRRACLTLHRHPTPHCSTWGLPLRVAGS WLTVVTVEALGGWRMGVRRTGQVGPTMHPPPVSGASPLLLHHLLLLLLIIILTC corresponding to amino acids 192-313 of MYEO_HUMAN_V2 (SEQ ID NO:681), which also corresponds to amino acids 1-122 of AA583399_PEA_{—}1_P5 (SEQ ID NO:686).

It should be noted that the known protein sequence (MYEO_HUMAN (SEQ ID NO:679)) has one or more changes than the sequence given at the end of the application and named as being the amino acid sequence for MYEO_HUMAN_V2 (SEQ ID NO:681). These changes were previously known to occur and are listed in the table below.

TABLE 14
Changes to MYEO_HUMAN_V2 (SEQ ID NO: 681)

| SNP position(s) on amino | |
|---|---|
| acid sequence | Type of change |
| 220 | conflict |

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein AA583399_PEA_{—}1_P5 (SEQ ID NO:686) also has the following non-silent SNPs (Single Nucleotide Polymorphisms) as listed in Table 15, (given according to their position(s) on the amino acid sequence, with the alternative amino acid(s) listed; the last column indicates whether the SNP is known or not; the presence of known SNPs in variant protein AA583399_PEA_{—}1_P5 (SEQ ID NO:686) sequence provides support for the deduced sequence of this variant protein according to the present invention).

TABLE 15
Amino acid mutations

| SNP position(s) on | Alternative | Previously |
|---|---|---|
| amino acid sequence | amino acid(s) | known SNP? |
| 7 | R -> Q | Yes |
| 28 | M -> V | No |
| 80 | G -> R | Yes |
| 93 | P -> T | Yes |

Variant protein AA583399_PEA_{—}1_P5 (SEQ ID NO:686) is encoded by the following transcript(s): AA583399_PEA_{—}1_T8 (SEQ ID NO:651), for which the sequence(s) is/are given at the end of the application. The coding portion of transcript AA583399_PEA_{—}1_T8 (SEQ ID NO:651) is shown in bold; this coding portion starts at position 849 and ends at position 1214. The transcript also has the following SNPs as listed in Table 16 (given according to their position on the nucleotide sequence, with the alternative nucleic acid listed; the last column indicates whether the SNP is known or not; the presence of known SNPs in variant protein AA583399_PEA_{—}1_P5 (SEQ ID NO:686) sequence provides support for the deduced sequence of this variant protein according to the present invention).

TABLE 16
Nucleic acid SNPs

| SNP position on | Alternative | Previously |
|---|---|---|
| nucleotide sequence | nucleic acid | known SNP? |
| 380 | A -> G | Yes |
| 494 | C -> T | Yes |
| 751 | T -> C | Yes |
| 868 | G -> A | Yes |
| 930 | A -> G | No |
| 1086 | G -> C | Yes |
| 1125 | C -> A | Yes |
| 1342 | C -> T | No |
| 1346 | T -> G | No |

Variant protein AA583399_PEA_{—}1_P6 (SEQ ID NO:687) according to the present invention has an amino acid sequence as given at the end of the application; it is encoded by transcript(s) AA583399_PEA_{—}1_T12 (SEQ ID NO:655). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein AA583399_PEA_{—}1_P6 (SEQ ID NO:687) is encoded by the following transcript(s): AA583399_PEA_{—}1_T12 (SEQ ID NO:655), for which the sequence(s) is/are given at the end of the application. The coding portion of transcript AA583399_PEA_{—}1_T12 (SEQ ID NO:655) is shown in bold; this coding portion starts at position 39 and ends at position 371. The transcript also has the following SNPs as listed in Table 17 (given according to their position on the nucleotide sequence, with the alternative nucleic acid listed; the last column indicates whether the SNP is known or not; the presence of known SNPs in variant protein AA583399_PEA_{—}1_P6 (SEQ ID NO:687) sequence provides support for the deduced sequence of this variant protein according to the present invention).

TABLE 17
Nucleic acid SNPs

| SNP position on | Alternative | Previously |
|---|---|---|
| nucleotide sequence | nucleic acid | known SNP? |
| 198 | A -> G | Yes |
| 538 | C -> A | Yes |
| 659 | C -> G | Yes |
| 1009 | C -> T | Yes |
| 1145 | A -> G | Yes |

Variant protein AA583399_PEA_{—}1_P8 (SEQ ID NO:688) according to the present invention has an amino acid sequence as given at the end of the application; it is encoded by transcript(s) AA583399_PEA_{—}1_T17 (SEQ ID NO:658). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein AA583399_PEA_{—}1_P8 (SEQ ID NO:688) is encoded by the following transcript(s): AA583399_PEA_{—}1_T17 (SEQ ID NO:658), for which the sequence(s) is/are given at the end of the application. The coding portion of transcript AA583399_PEA_{—}1_T17 (SEQ ID NO:658) is shown in bold; this coding portion starts at position 191 and ends at position 400.

Variant protein AA583399_PEA_{—}1_P10 (SEQ ID NO:689) according to the present invention has an amino acid sequence as given at the end of the application; it is encoded by transcript(s) AA583399_PEA_{—}1_T0 (SEQ ID NO:643). An alignment is given to the known protein (Myeloma overexpressed gene protein (SEQ ID NO:679)) at the end of the application. One or more alignments to one or more previously published protein sequences are given at the end of the application. A brief description of the relationship of the variant protein according to the present invention to each such aligned protein is as follows:

Comparison Report Between AA583399_PEA_{—}1_P10 (SEQ ID NO:689) and MYEO_HUMAN_V3 (SEQ ID NO: 682):

1. An isolated chimeric polypeptide encoding for AA583399_PEA_{—}1_P10 (SEQ ID NO:689), comprising a first amino acid sequence being at least 90% homologous to MFTRQAGHFVEGSKAGRSRGRLCLSQALRVAVRGAFVSLWFAAGAGDRERNKGDKGAQTGAGLSQEAE DVDVSRARRVTDAPQGTLCGTGNRNSGSQSARAVGVAHLGEAFRVGVEQAISSCPEEVHGRHGLSMEIM WAQMDVALRSPGRGLLAGAGALCMTLAESSCPDYERGRRACLTLHRHPTPHCSTWGLPLRVAGSWLTV VTVEALGRWRMGVRRTGQVGPTMHPPPVSGASPLLLHHLLLLLLIIILTC corresponding to amino acids 59-313 of MYEO_HUMAN_V3 (SEQ ID NO:682), which also corresponds to amino acids 1-255 of AA583399_PEA_{—}1_P10 (SEQ ID NO:689).

It should be noted that the known protein sequence (MYEO_HUMAN (SEQ ID NO:679)) has one or more changes than the sequence given at the end of the application and named as being the amino acid sequence for MYEO_HUMAN_V3 (SEQ ID NO:682). These changes were previously known to occur and are listed in the table below.

TABLE 18
Changes to MYEO_HUMAN_V3 (SEQ ID NO: 682)

| SNP position(s) on amino | |
|---|---|
| acid sequence | Type of change |
| 199 | conflict |
| 220 | conflict |
| 272 | conflict |

The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein AA583399_PEA_{—}1_P10 (SEQ ID NO:689) also has the following non-silent SNPs (Single Nucleotide Polymorphisms) as listed in Table 19, (given according to their position(s) on the amino acid sequence, with the alternative amino acid(s) listed; the last column indicates whether the SNP is known or not; the presence of known SNPs in variant protein AA583399_PEA_{—}1_P10 (SEQ ID NO:689) sequence provides support for the deduced sequence of this variant protein according to the present invention).

TABLE 19
Amino acid mutations

| SNP position(s) on | Alternative | Previously |
|---|---|---|
| amino acid sequence | amino acid(s) | known SNP? |
| 101 | A -> V | Yes |
| 140 | Q -> R | Yes |
| 161 | M -> V | No |
| 213 | R -> G | Yes |
| 226 | P -> T | Yes |

Variant protein AA583399_PEA_{—}1_P10 (SEQ ID NO:689) is encoded by the following transcript(s): AA583399_PEA_{—}1_T0 (SEQ ID NO:643), for which the sequence(s) is/are given at the end of the application. The coding portion of transcript AA583399_PEA_{—}1_T0 (SEQ ID NO:643) is shown in bold; this coding portion starts at position 857 and ends at position 1621. The transcript also has the following SNPs as listed in Table 20 (given according to their position on the nucleotide sequence, with the alternative nucleic acid listed; the last column indicates whether the SNP is known or not; the presence of known SNPs in variant protein AA583399_PEA_{—}1_P10 (SEQ ID NO:689) sequence provides support for the deduced sequence of this variant protein according to the present invention).

TABLE 20
Nucleic acid SNPs

| SNP position on | Alternative | Previously |
|---|---|---|
| nucleotide sequence | nucleic acid | known SNP? |
| 380 | A -> G | Yes |
| 901 | C -> T | Yes |
| 1158 | T -> C | Yes |
| 1275 | G -> A | Yes |
| 1337 | A -> G | No |
| 1493 | G -> C | Yes |
| 1532 | C -> A | Yes |
| 1749 | C -> T | No |
| 1753 | T -> G | No |

Variant protein AA583399_PEA_{—}1_P11 (SEQ ID NO:690) according to the present invention has an amino acid sequence as given at the end of the application; it is encoded by transcript(s) AA583399_PEA_{—}1_T2 (SEQ ID NO:645). The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide.

Variant protein AA583399_PEA_{—}1_P11 (SEQ ID NO:690) also has the following non-silent SNPs (Single Nucleotide Polymorphisms) as listed in Table 21, (given according to their position(s) on the amino acid sequence, with the alternative amino acid(s) listed; the last column indicates whether the SNP is known or not; the presence of known SNPs in variant protein AA583399_PEA_{—}1_P11 (SEQ ID NO:690) sequence provides support for the deduced sequence of this variant protein according to the present invention).

TABLE 21
Amino acid mutations

| SNP position(s) on | Alternative | Previously |
|---|---|---|
| amino acid sequence | amino acid(s) | known SNP? |
| 159 | A -> V | Yes |
| 198 | R -> Q | Yes |
| 219 | V -> M | No |
| 271 | G -> R | Yes |
| 284 | P -> T | Yes |

Variant protein AA583399_PEA_{—}1_P11 (SEQ ID NO:690) is encoded by the following transcript(s): AA583399_PEA_{—}1_T2 (SEQ ID NO:645), for which the sequence(s) is/are given at the end of the application. The coding portion of transcript AA583399_PEA_{—}1_T2 (SEQ ID NO:645) is shown in bold; this coding portion starts at position 493 and ends at position 1431. The transcript also has the following SNPs as listed in Table 22 (given according to their position on the nucleotide sequence, with the alternative nucleic acid listed; the last column indicates whether the SNP is known or not; the presence of known SNPs in variant protein AA583399_PEA_{—}1_P11 (SEQ ID NO:690) sequence provides support for the deduced sequence of this variant protein according to the present invention).

TABLE 22
Nucleic acid SNPs

| SNP position on | Alternative | Previously |
|---|---|---|
| nucleotide sequence | nucleic acid | known SNP? |
| 380 | A -> G | Yes |
| 711 | C -> T | Yes |
| 968 | T -> C | Yes |
| 1085 | G -> A | Yes |
| 1147 | A -> G | No |
| 1303 | G -> C | Yes |
| 1342 | C -> A | Yes |
| 1559 | C -> T | No |
| 1563 | T -> G | No |

Variant protein AA583399_PEA_{—}1_P12 (SEQ ID NO:691) according to the present invention has an amino acid sequence as given at the end of the application; it is encoded by transcript(s) AA583399_PEA_{—}1_T10 (SEQ ID NO:653) and AA583399_PEA_{—}1_T11 (SEQ ID NO:654). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein AA583399_PEA_{—}1_P12 (SEQ ID NO:691) is encoded by the following transcript(s): AA583399_PEA_{—}1_T10 (SEQ ID NO:653) and AA583399_PEA_{—}1_T11 (SEQ ID NO:654), for which the sequence(s) is/are given at the end of the application.

The coding portion of transcript AA583399_PEA_{—}1_T10 (SEQ ID NO:653) is shown in bold; this coding portion starts at position 191 and ends at position 367. The transcript also has the following SNPs as listed in Table 23 (given according to their position on the nucleotide sequence, with the alternative nucleic acid listed; the last column indicates whether the SNP is known or not; the presence of known SNPs in variant protein AA583399_PEA_{—}1_P12 (SEQ ID NO:691) sequence provides support for the deduced sequence of this variant protein according to the present invention).

TABLE 23
Nucleic acid SNPs

| SNP position on | Alternative | Previously |
|---|---|---|
| nucleotide sequence | nucleic acid | known SNP? |
| 380 | A -> G | Yes |

The coding portion of transcript AA583399_PEA_{—}1_T11 (SEQ ID NO:654) is shown in bold; this coding portion starts at position 191 and ends at position 367. The transcript also has the following SNPs as listed in Table 24 (given according to their position on the nucleotide sequence, with the alternative nucleic acid listed; the last column indicates whether the SNP is known or not; the presence of known SNPs in variant protein AA583399_PEA_{—}1_P12 (SEQ ID NO:691) sequence provides support for the deduced sequence of this variant protein according to the present invention).

TABLE 24
Nucleic acid SNPs

| SNP position on nucleotide | Alternative | |
|---|---|---|
| sequence | nucleic acid | Previously known SNP? |
| 380 | A -> G | Yes |

Variant protein AA583399_PEA_{—}1_P14 (SEQ ID NO:692) according to the present invention has an amino acid sequence as given at the end of the application; it is encoded by transcript(s) AA583399_PEA_{—}1_T15 (SEQ ID NO:656). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein AA583399_PEA_{—}1_P14 (SEQ ID NO:692) is encoded by the following transcript(s): AA583399_PEA_{—}1_T15 (SEQ ID NO:656), for which the sequence(s) is/are given at the end of the application. The coding portion of transcript AA583399_PEA_{—}1_T15 (SEQ ID NO:656) is shown in bold; this coding portion starts at position 43 and ends at position 210.

As noted above, cluster AA583399 features 20 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster AA583399_PEA_{—}1_node_{—}0 (SEQ ID NO:659) according to the present invention is supported by 24 libraries. The number of libraries was determined as previously described. This segment can be found in the following transcript(s): AA583399_PEA_{—}1_T0 (SEQ ID NO:643), AA583399_PEA_{—}1_T1 (SEQ ID NO:644), AA583399_PEA_{—}1_T2 (SEQ ID NO:645), AA583399_PEA_{—}1_T3 (SEQ ID NO:646), AA583399_PEA_{—}1_T4 (SEQ ID NO:647), AA583399_PEA_{—}1_T5 (SEQ ID NO:648), AA583399_PEA_{—}1_T6 (SEQ ID NO:649), AA583399_PEA_{—}1_T7 (SEQ ID NO:650), AA583399_PEA_{—}1_T8 (SEQ ID NO:651), AA583399_PEA_{—}1_T9 (SEQ ID NO:652), AA583399_PEA_{—}1_T10 (SEQ ID NO:653), AA583399_PEA_{—}1_T11 (SEQ ID NO:654) and AA583399_PEA_{—}1_T17 (SEQ ID NO:658). Table 25 below describes the starting and ending position of this segment on each transcript.

TABLE 25
Segment location on transcripts

| | Segment | Segment |
|---|---|---|
| | starting | ending |
| Transcript name | position | position |
| AA583399_PEA_1_T0 (SEQ ID NO: 643) | 1 | 355 |
| AA583399_PEA_1_T1 (SEQ ID NO: 644) | 1 | 355 |
| AA583399_PEA_1_T2 (SEQ ID NO: 645) | 1 | 355 |
| AA583399_PEA_1_T3 (SEQ ID NO: 646) | 1 | 355 |
| AA583399_PEA_1_T4 (SEQ ID NO: 647) | 1 | 355 |
| AA583399_PEA_1_T5 (SEQ ID NO: 648) | 1 | 355 |
| AA583399_PEA_1_T6 (SEQ ID NO: 649) | 1 | 355 |
| AA583399_PEA_1_T7 (SEQ ID NO: 650) | 1 | 355 |
| AA583399_PEA_1_T8 (SEQ ID NO: 651) | 1 | 355 |
| AA583399_PEA_1_T9 (SEQ ID NO: 652) | 1 | 355 |
| AA583399_PEA_1_T10 (SEQ ID NO: 653) | 1 | 355 |
| AA583399_PEA_1_T11 (SEQ ID NO: 654) | 1 | 355 |
| AA583399_PEA_1_T17 (SEQ ID NO: 658) | 1 | 355 |

Segment cluster AA583399_PEA_{—}1_node_{—}3 (SEQ ID NO:660) according to the present invention is supported by 4 libraries. The number of libraries was determined as previously described. This segment can be found in the following transcript(s): AA583399_PEA_{—}1_T4 (SEQ ID NO:647). Table 26 below describes the starting and ending position of this segment on each transcript.

TABLE 26
Segment location on transcripts

| | Segment | |
|---|---|---|
| | starting | Segment |
| Transcript name | position | ending position |
| AA583399_PEA_1_T4 (SEQ ID NO: 647) | 465 | 1120 |

Segment cluster AA583399_PEA_{—}1_node_{—}9 (SEQ ID NO:661) according to the present invention is supported by 23 libraries. The number of libraries was determined as previously described. This segment can be found in the following transcript(s): AA583399_PEA_{—}1_T0 (SEQ ID NO:643), AA583399_PEA_{—}1_T1 (SEQ ID NO:644), AA583399_PEA_{—}1_T2 (SEQ ID NO:645), AA583399_PEA_{—}1_T3 (SEQ ID NO:646), AA583399_PEA_{—}1_T4 (SEQ ID NO:647), AA583399_PEA_{—}1_T5 (SEQ ID NO:648), AA583399_PEA_{—}1_T6 (SEQ ID NO:649), AA583399_PEA_{—}1_T8 (SEQ ID NO:651) and AA583399_PEA_{—}1_T9 (SEQ ID NO:652). Table 27 below describes the starting and ending position of this segment on each transcript.

TABLE 27
Segment location on transcripts

| | Segment | |
|---|---|---|
| | starting | Segment |
| Transcript name | position | ending position |
| AA583399_PEA_1_T0 (SEQ ID NO: 643) | 872 | 1131 |
| AA583399_PEA_1_T1 (SEQ ID NO: 644) | 776 | 1035 |
| AA583399_PEA_1_T2 (SEQ ID NO: 645) | 682 | 941 |
| AA583399_PEA_1_T3 (SEQ ID NO: 646) | 704 | 963 |
| AA583399_PEA_1_T4 (SEQ ID NO: 647) | 1528 | 1787 |
| AA583399_PEA_1_T5 (SEQ ID NO: 648) | 778 | 1037 |
| AA583399_PEA_1_T6 (SEQ ID NO: 649) | 776 | 1035 |
| AA583399_PEA_1_T8 (SEQ ID NO: 651) | 465 | 724 |
| AA583399_PEA_1_T9 (SEQ ID NO: 652) | 776 | 1035 |

Segment cluster AA583399_PEA_{—}1_node_{—}10 (SEQ ID NO:662) according to the present invention is supported by 59 libraries. The number of libraries was determined as previously described. This segment can be found in the following transcript(s): AA583399_PEA_{—}1_T0 (SEQ ID NO:643), AA583399_PEA_{—}1_T1 (SEQ ID NO:644), AA583399_PEA_{—}1_T2 (SEQ ID NO:645), AA583399_PEA_{—}1_T3 (SEQ ID NO:646), AA583399_PEA_{—}1_T4 (SEQ ID NO:647), AA583399_PEA_{—}1_T5 (SEQ ID NO:648), AA583399_PEA_{—}1_T6 (SEQ ID NO:649), AA583399_PEA_{—}1_T7 (SEQ ID NO:650), AA583399_PEA_{—}1_T8 (SEQ ID NO:651) and AA583399_PEA_{—}1_T9 (SEQ ID NO:652). Table 28 below describes the starting and ending position of this segment on each transcript.

TABLE 28
Segment location on transcripts

| | Segment | |
|---|---|---|
| | starting | Segment |
| Transcript name | position | ending position |
| AA583399_PEA_1_T0 (SEQ ID NO: 643) | 1132 | 2389 |
| AA583399_PEA_1_T1 (SEQ ID NO: 644) | 1036 | 2293 |
| AA583399_PEA_1_T2 (SEQ ID NO: 645) | 942 | 2199 |
| AA583399_PEA_1_T3 (SEQ ID NO: 646) | 964 | 2221 |
| AA583399_PEA_1_T4 (SEQ ID NO: 647) | 1788 | 3045 |
| AA583399_PEA_1_T5 (SEQ ID NO: 648) | 1038 | 2295 |
| AA583399_PEA_1_T6 (SEQ ID NO: 649) | 1036 | 2293 |
| AA583399_PEA_1_T7 (SEQ ID NO: 650) | 872 | 2129 |
| AA583399_PEA_1_T8 (SEQ ID NO: 651) | 725 | 1982 |
| AA583399_PEA_1_T9 (SEQ ID NO: 652) | 1036 | 2293 |

Segment cluster AA583399_PEA_{—}1_node_{—}12 (SEQ ID NO:663) according to the present invention is supported by 34 libraries. The number of libraries was determined as previously described. This segment can be found in the following transcript(s): AA583399_PEA_{—}1_T0 (SEQ ID NO:643), AA583399_PEA_{—}1_T1 (SEQ ID NO:644), AA583399_PEA_{—}1_T2 (SEQ ID NO:645), AA583399_PEA_{—}1_T3 (SEQ ID NO:646), AA583399_PEA_{—}1_T4 (SEQ ID NO:647), AA583399_PEA_{—}1_T5 (SEQ ID NO:648), AA583399_PEA_{—}1_T6 (SEQ ID NO:649), AA583399_PEA_{—}1_T7 (SEQ ID NO:650), AA583399_PEA_{—}1_T8 (SEQ ID NO:651) and AA583399_PEA_{—}1_T9 (SEQ ID NO:652). Table 29 below describes the starting and ending position of this segment on each transcript.

TABLE 29
Segment location on transcripts

| | Segment | |
|---|---|---|
| | starting | Segment |
| Transcript name | position | ending position |
| AA583399_PEA_1_T0 (SEQ ID NO: 643) | 2412 | 2519 |
| AA583399_PEA_1_T1 (SEQ ID NO: 644) | 2316 | 2423 |
| AA583399_PEA_1_T2 (SEQ ID NO: 645) | 2222 | 2329 |
| AA583399_PEA_1_T3 (SEQ ID NO: 646) | 2244 | 2351 |
| AA583399_PEA_1_T4 (SEQ ID NO: 647) | 3068 | 3175 |
| AA583399_PEA_1_T5 (SEQ ID NO: 648) | 2318 | 2425 |
| AA583399_PEA_1_T6 (SEQ ID NO: 649) | 2316 | 2589 |
| AA583399_PEA_1_T7 (SEQ ID NO: 650) | 2152 | 2259 |
| AA583399_PEA_1_T8 (SEQ ID NO: 651) | 2005 | 2112 |
| AA583399_PEA_1_T9 (SEQ ID NO: 652) | 2294 | 2401 |

Segment cluster AA583399_PEA_{—}1_node_{—}14 (SEQ ID NO:664) according to the present invention is supported by 6 libraries. The number of libraries was determined as previously described. This segment can be found in the following transcript(s): AA583399_PEA_{—}1_T12 (SEQ ID NO:655) and AA583399_PEA_{—}1_T16 (SEQ ID NO:657). Table 30 below describes the starting and ending position of this segment on each transcript.

TABLE 30
Segment location on transcripts

| | Segment | Segment |
|---|---|---|
| | starting | ending |
| Transcript name | position | position |
| AA583399_PEA_1_T12 (SEQ ID NO: 655) | 1 | 1179 |
| AA583399_PEA_1_T16 (SEQ ID NO: 657) | 1 | 1179 |

Segment cluster AA583399_PEA_{—}1_node_{—}21 (SEQ ID NO:665) according to the present invention is supported by 15 libraries. The number of libraries was determined as previously described. This segment can be found in the following transcript(s): AA583399_PEA_{—}1_T10 (SEQ ID NO:653), AA583399_PEA_{—}1_T11 (SEQ ID NO:654), AA583399_PEA_{—}1_T12 (SEQ ID NO:655), AA583399_PEA_{—}1_T15 (SEQ ID NO:656), AA583399_PEA_{—}1_T16 (SEQ ID NO:657) and AA583399_PEA_{—}1_T17 (SEQ ID NO:658). Table 31 below describes the starting and ending position of this segment on each transcript.

TABLE 31
Segment location on transcripts

| | Segment | Segment |
|---|---|---|
| | starting | ending |
| Transcript name | position | position |
| AA583399_PEA_1_T10 (SEQ ID NO: 653) | 465 | 633 |
| AA583399_PEA_1_T11 (SEQ ID NO: 654) | 465 | 633 |
| AA583399_PEA_1_T12 (SEQ ID NO: 655) | 1180 | 1348 |
| AA583399_PEA_1_T15 (SEQ ID NO: 656) | 78 | 246 |
| AA583399_PEA_1_T16 (SEQ ID NO: 657) | 1180 | 1348 |
| AA583399_PEA_1_T17 (SEQ ID NO: 658) | 434 | 602 |

Segment cluster AA583399_PEA_{—}1_node_{—}24 (SEQ ID NO:666) according to the present invention is supported by 7 libraries. The number of libraries was determined as previously described. This segment can be found in the following transcript(s): AA583399_PEA_{—}1_T11 (SEQ ID NO:654), AA583399_PEA_{—}1_T12 (SEQ ID NO:655), AA583399_PEA_{—}1_T15 (SEQ ID NO:656) and AA583399_PEA_{—}1_T16 (SEQ ID NO:657). Table 32 below describes the starting and ending position of this segment on each transcript.

TABLE 32
Segment location on transcripts

| | Segment | Segment |
|---|---|---|
| | starting | ending |
| Transcript name | position | position |
| AA583399_PEA_1_T11 (SEQ ID NO: 654) | 634 | 757 |
| AA583399_PEA_1_T12 (SEQ ID NO: 655) | 1349 | 1472 |
| AA583399_PEA_1_T15 (SEQ ID NO: 656) | 247 | 370 |
| AA583399_PEA_1_T16 (SEQ ID NO: 657) | 1349 | 1472 |

Segment cluster AA583399_PEA_{—}1_node_{—}25 (SEQ ID NO:667) according to the present invention is supported by 1 libraries. The number of libraries was determined as previously described. This segment can be found in the following transcript(s): AA583399_PEA_{—}1_T16 (SEQ ID NO:657). Table 33 below describes the starting and ending position of this segment on each transcript.

TABLE 33
Segment location on transcripts

| | Segment | Segment |
|---|---|---|
| Transcript name | starting position | ending position |
| AA583399_PEA_1_T16 (SEQ ID | 1473 | 1614 |
| NO: 657) | | |

Segment cluster AA583399_PEA_{—}1_node_{—}29 (SEQ ID NO:668) according to the present invention is supported by 8 libraries. The number of libraries was determined as previously described. This segment can be found in the following transcript(s): AA583399_PEA_{—}1_T11 (SEQ ID NO:654), AA583399_PEA_{—}1_T12 (SEQ ID NO:655) and AA583399_PEA_{—}1_T15 (SEQ ID NO:656). Table 34 below describes the starting and ending position of this segment on each transcript.

TABLE 34
Segment location on transcripts

| | Segment | Segment |
|---|---|---|
| | starting | ending |
| Transcript name | position | position |
| AA583399_PEA_1_T11 (SEQ ID NO: 654) | 832 | 1491 |
| AA583399_PEA_1_T12 (SEQ ID NO: 655) | 1547 | 2206 |
| AA583399_PEA_1_T15 (SEQ ID NO: 656) | 445 | 1104 |

According to an optional embodiment of the present invention, short segments related to the above cluster are also provided. These segments are up to about 120 bp in length, and so are included in a separate description.

Segment cluster AA583399_PEA_{—}1_node_{—}1 (SEQ ID NO:669) according to the present invention is supported by 22 libraries. The number of libraries was determined as previously described. This segment can be found in the following transcript(s): AA583399_PEA_{—}1_T0 (SEQ ID NO:643), AA583399_PEA_{—}1_T1 (SEQ ID NO:644), AA583399_PEA_{—}1_T2 (SEQ ID NO:645), AA583399_PEA_{—}1_T3 (SEQ ID NO:646), AA583399_PEA_{—}1_T4 (SEQ ID NO:647), AA583399_PEA_{—}1_T5 (SEQ ID NO:648), AA583399_PEA_{—}1_T6 (SEQ ID NO:649), AA583399_PEA_{—}1_T7 (SEQ ID NO:650), AA583399_PEA_{—}1_T8 (SEQ ID NO:651), AA583399_PEA_{—}1_T9 (SEQ ID NO:652), AA583399_PEA_{—}1_T10 (SEQ ID NO:653) and AA583399_PEA_{—}1_T11 (SEQ ID NO:654). Table 35 below describes the starting and ending position of this segment on each transcript.

TABLE 35
Segment location on transcripts

| | Segment | Segment |
|---|---|---|
| | starting | ending |
| Transcript name | position | position |
| AA583399_PEA_1_T0 (SEQ ID NO: 643) | 356 | 386 |
| AA583399_PEA_1_T1 (SEQ ID NO: 644) | 356 | 386 |
| AA583399_PEA_1_T2 (SEQ ID NO: 645) | 356 | 386 |
| AA583399_PEA_1_T3 (SEQ ID NO: 646) | 356 | 386 |
| AA583399_PEA_1_T4 (SEQ ID NO: 647) | 356 | 386 |
| AA583399_PEA_1_T5 (SEQ ID NO: 648) | 356 | 386 |
| AA583399_PEA_1_T6 (SEQ ID NO: 649) | 356 | 386 |
| AA583399_PEA_1_T7 (SEQ ID NO: 650) | 356 | 386 |
| AA583399_PEA_1_T8 (SEQ ID NO: 651) | 356 | 386 |
| AA583399_PEA_1_T9 (SEQ ID NO: 652) | 356 | 386 |
| AA583399_PEA_1_T10 (SEQ ID NO: 653) | 356 | 386 |
| AA583399_PEA_1_T11 (SEQ ID NO: 654) | 356 | 386 |

Segment cluster AA583399_PEA_{—}1_node_{—}2 (SEQ ID NO:670) according to the present invention is supported by 24 libraries. The number of libraries was determined as previously described. This segment can be found in the following transcript(s): AA583399_PEA_{—}1_T0 (SEQ ID NO:643), AA583399_PEA_{—}1_T1 (SEQ ID NO:644), AA583399_PEA_{—}1_T2 (SEQ ID NO:645), AA583399_PEA_{—}1_T3 (SEQ ID NO:646), AA583399_PEA_{—}1_T4 (SEQ ID NO:647), AA583399_PEA_{—}1_T5 (SEQ ID NO:648), AA583399_PEA_{—}1_T6 (SEQ ID NO:649), AA583399_PEA_{—}1_T7 (SEQ ID NO:650), AA583399_PEA_{—}1_T8 (SEQ ID NO:651), AA583399_PEA_{—}1_T9 (SEQ ID NO:652), AA583399_PEA_{—}1_T10 (SEQ ID NO:653), AA583399_PEA_{—}1_T11 (SEQ ID NO:654) and AA583399_PEA_{—}1_T17 (SEQ ID NO:658). Table 36 below describes the starting and ending position of this segment on each transcript.

TABLE 36
Segment location on transcripts

| | Segment | Segment |
|---|---|---|
| | starting | ending |
| Transcript name | position | position |
| AA583399_PEA_1_T0 (SEQ ID NO: 643) | 387 | 464 |
| AA583399_PEA_1_T1 (SEQ ID NO: 644) | 387 | 464 |
| AA583399_PEA_1_T2 (SEQ ID NO: 645) | 387 | 464 |
| AA583399_PEA_1_T3 (SEQ ID NO: 646) | 387 | 464 |
| AA583399_PEA_1_T4 (SEQ ID NO: 647) | 387 | 464 |
| AA583399_PEA_1_T5 (SEQ ID NO: 648) | 387 | 464 |
| AA583399_PEA_1_T6 (SEQ ID NO: 649) | 387 | 464 |
| AA583399_PEA_1_T7 (SEQ ID NO: 650) | 387 | 464 |
| AA583399_PEA_1_T8 (SEQ ID NO: 651) | 387 | 464 |
| AA583399_PEA_1_T9 (SEQ ID NO: 652) | 387 | 464 |
| AA583399_PEA_1_T10 (SEQ ID NO: 653) | 387 | 464 |
| AA583399_PEA_1_T11 (SEQ ID NO: 654) | 387 | 464 |
| AA583399_PEA_1_T17 (SEQ ID NO: 658) | 356 | 433 |

Segment cluster AA583399_PEA_{—}1_node_{—}4 (SEQ ID NO:671) according to the present invention is supported by 13 libraries. The number of libraries was determined as previously described. This segment can be found in the following transcript(s): AA583399_PEA_{—}1_T0 (SEQ ID NO:643), AA583399_PEA_{—}1_T1 (SEQ ID NO:644), AA583399_PEA_{—}1_T4 (SEQ ID NO:647), AA583399_PEA_{—}1_T6 (SEQ ID NO:649), AA583399_PEA_{—}1_T7 (SEQ ID NO:650) and AA583399_PEA_{—}1_T9 (SEQ ID NO:652). Table 37 below describes the starting and ending position of this segment on each transcript.

TABLE 37
Segment location on transcripts

| | Segment | |
|---|---|---|
| | starting | Segment |
| Transcript name | position | ending position |
| AA583399_PEA_1_T0 (SEQ ID NO: 643) | 465 | 558 |
| AA583399_PEA_1_T1 (SEQ ID NO: 644) | 465 | 558 |
| AA583399_PEA_1_T4 (SEQ ID NO: 647) | 1121 | 1214 |
| AA583399_PEA_1_T6 (SEQ ID NO: 649) | 465 | 558 |
| AA583399_PEA_1_T7 (SEQ ID NO: 650) | 465 | 558 |
| AA583399_PEA_1_T9 (SEQ ID NO: 652) | 465 | 558 |

Segment cluster AA583399_PEA_{—}1_node_{—}5 (SEQ ID NO:672) according to the present invention is supported by 13 libraries. The number of libraries was determined as previously described. This segment can be found in the following transcript(s): AA583399_PEA_{—}1_T0 (SEQ ID NO:643), AA583399_PEA_{—}1_T1 (SEQ ID NO:644), AA583399_PEA_{—}1_T2 (SEQ ID NO:645), AA583399_PEA_{—}1_T4 (SEQ ID NO:647), AA583399_PEA_{—}1_T5 (SEQ ID NO:648), AA583399_PEA_{—}1_T6 (SEQ ID NO:649), AA583399_PEA_{—}1_T7 (SEQ ID NO:650) and AA583399_PEA_{—}1_T9 (SEQ ID NO:652). Table 38 below describes the starting and ending position of this segment on each transcript.

TABLE 38
Segment location on transcripts

| | Segment | |
|---|---|---|
| | starting | Segment |
| Transcript name | position | ending position |
| AA583399_PEA_1_T0 (SEQ ID NO: 643) | 559 | 632 |
| AA583399_PEA_1_T1 (SEQ ID NO: 644) | 559 | 632 |
| AA583399_PEA_1_T2 (SEQ ID NO: 645) | 465 | 538 |
| AA583399_PEA_1_T4 (SEQ ID NO: 647) | 1215 | 1288 |
| AA583399_PEA_1_T5 (SEQ ID NO: 648) | 465 | 538 |
| AA583399_PEA_1_T6 (SEQ ID NO: 649) | 559 | 632 |
| AA583399_PEA_1_T7 (SEQ ID NO: 650) | 559 | 632 |
| AA583399_PEA_1_T9 (SEQ ID NO: 652) | 559 | 632 |

Segment cluster AA583399_PEA_{—}1_node_{—}6 (SEQ ID NO:673) according to the present invention is supported by 13 libraries. The number of libraries was determined as previously described. This segment can be found in the following transcript(s): AA583399_PEA_{—}1_T0 (SEQ ID NO:643), AA583399_PEA_{—}1_T1 (SEQ ID NO:644), AA583399_PEA_{—}1_T2 (SEQ ID NO:645), AA583399_PEA_{—}1_T3 (SEQ ID NO:646), AA583399_PEA_{—}1_T4 (SEQ ID NO:647), AA583399_PEA_{—}1_T5 (SEQ ID NO:648), AA583399_PEA_{—}1_T6 (SEQ ID NO:649), AA583399_PEA_{—}1_T7 (SEQ ID NO:650) and AA583399_PEA_{—}1_T9 (SEQ ID NO:652). Table 39 below describes the starting and ending position of this segment on each transcript.

TABLE 39
Segment location on transcripts

| | Segment | |
|---|---|---|
| | starting | Segment |
| Transcript name | position | ending position |
| AA583399_PEA_1_T0 (SEQ ID NO: 643) | 633 | 727 |
| AA583399_PEA_1_T1 (SEQ ID NO: 644) | 633 | 727 |
| AA583399_PEA_1_T2 (SEQ ID NO: 645) | 539 | 633 |
| AA583399_PEA_1_T3 (SEQ ID NO: 646) | 465 | 559 |
| AA583399_PEA_1_T4 (SEQ ID NO: 647) | 1289 | 1383 |
| AA583399_PEA_1_T5 (SEQ ID NO: 648) | 539 | 633 |
| AA583399_PEA_1_T6 (SEQ ID NO: 649) | 633 | 727 |
| AA583399_PEA_1_T7 (SEQ ID NO: 650) | 633 | 727 |
| AA583399_PEA_1_T9 (SEQ ID NO: 652) | 633 | 727 |

Segment cluster AA583399_PEA_{—}1_node_{—}7 (SEQ ID NO:674) according to the present invention is supported by 9 libraries. The number of libraries was determined as previously described. This segment can be found in the following transcript(s): AA583399_PEA_{—}1_T0 (SEQ ID NO:643), AA583399_PEA_{—}1_T3 (SEQ ID NO:646), AA583399_PEA_{—}1_T4 (SEQ ID NO:647), AA583399_PEA_{—}1_T5 (SEQ ID NO:648) and AA583399_PEA_{—}1_T7 (SEQ ID NO:650). Table 40 below describes the starting and ending position of this segment on each transcript.

TABLE 40
Segment location on transcripts

| | Segment | |
|---|---|---|
| | starting | Segment |
| Transcript name | position | ending position |
| AA583399_PEA_1_T0 (SEQ ID NO: 643) | 728 | 823 |
| AA583399_PEA_1_T3 (SEQ ID NO: 646) | 560 | 655 |
| AA583399_PEA_1_T4 (SEQ ID NO: 647) | 1384 | 1479 |
| AA583399_PEA_1_T5 (SEQ ID NO: 648) | 634 | 729 |
| AA583399_PEA_1_T7 (SEQ ID NO: 650) | 728 | 823 |

Segment cluster AA583399_PEA_{—}1_node_{—}8 (SEQ ID NO:675) according to the present invention is supported by 13 libraries. The number of libraries was determined as previously described. This segment can be found in the following transcript(s): AA583399_PEA_{—}1_T0 (SEQ ID NO:643), AA583399_PEA_{—}1_T1 (SEQ ID NO:644), AA583399_PEA_{—}1_T2 (SEQ ID NO:645), AA583399_PEA_{—}1_T3 (SEQ ID NO:646), AA583399_PEA_{—}1_T4 (SEQ ID NO:647), AA583399_PEA_{—}1_T5 (SEQ ID NO:648), AA583399_PEA_{—}1_T6 (SEQ ID NO:649), AA583399_PEA_{—}1_T7 (SEQ ID NO:650) and AA583399_PEA_{—}1_T9 (SEQ ID NO:652). Table 41 below describes the starting and ending position of this segment on each transcript.

TABLE 41
Segment location on transcripts

| | Segment | |
|---|---|---|
| | starting | Segment |
| Transcript name | position | ending position |
| AA583399_PEA_1_T0 (SEQ ID NO: 643) | 824 | 871 |
| AA583399_PEA_1_T1 (SEQ ID NO: 644) | 728 | 775 |
| AA583399_PEA_1_T2 (SEQ ID NO: 645) | 634 | 681 |
| AA583399_PEA_1_T3 (SEQ ID NO: 646) | 656 | 703 |
| AA583399_PEA_1_T4 (SEQ ID NO: 647) | 1480 | 1527 |
| AA583399_PEA_1_T5 (SEQ ID NO: 648) | 730 | 777 |
| AA583399_PEA_1_T6 (SEQ ID NO: 649) | 728 | 775 |
| AA583399_PEA_1_T7 (SEQ ID NO: 650) | 824 | 871 |
| AA583399_PEA_1_T9 (SEQ ID NO: 652) | 728 | 775 |

Segment cluster AA583399_PEA_{—}1_node_{—}11 (SEQ ID NO:676) according to the present invention can be found in the following transcript(s): AA583399_PEA_{—}1_T0 (SEQ ID NO:643), AA583399_PEA_{—}1_T1 (SEQ ID NO:644), AA583399_PEA_{—}1_T2 (SEQ ID NO:645), AA583399_PEA_{—}1_T3 (SEQ ID NO:646), AA583399_PEA_{—}1_T4 (SEQ ID NO:647), AA583399_PEA_{—}1_T5 (SEQ ID NO:648), AA583399_PEA_{—}1_T6 (SEQ ID NO:649), AA583399_PEA_{—}1_T7 (SEQ ID NO:650) and AA583399_PEA_{—}1_T8 (SEQ ID NO:651). Table 42 below describes the starting and ending position of this segment on each transcript.

TABLE 42
Segment location on transcripts

| | Segment | |
|---|---|---|
| | starting | Segment |
| Transcript name | position | ending position |
| AA583399_PEA_1_T0 (SEQ ID NO: 643) | 2390 | 2411 |
| AA583399_PEA_1_T1 (SEQ ID NO: 644) | 2294 | 2315 |
| AA583399_PEA_1_T2 (SEQ ID NO: 645) | 2200 | 2221 |
| AA583399_PEA_1_T3 (SEQ ID NO: 646) | 2222 | 2243 |
| AA583399_PEA_1_T4 (SEQ ID NO: 647) | 3046 | 3067 |
| AA583399_PEA_1_T5 (SEQ ID NO: 648) | 2296 | 2317 |
| AA583399_PEA_1_T6 (SEQ ID NO: 649) | 2294 | 2315 |
| AA583399_PEA_1_T7 (SEQ ID NO: 650) | 2130 | 2151 |
| AA583399_PEA_1_T8 (SEQ ID NO: 651) | 1983 | 2004 |

Segment cluster AA583399_PEA_{—}1_node_{—}19 (SEQ ID NO:677) according to the present invention is supported by 1 libraries. The number of libraries was determined as previously described. This segment can be found in the following transcript(s): AA583399_PEA_{—}1_T15 (SEQ ID NO:656). Table 43 below describes the starting and ending position of this segment on each transcript.

TABLE 43
Segment location on transcripts

| | Segment | Segment |
|---|---|---|
| | starting | ending |
| Transcript name | position | position |
| AA583399_PEA_1_T15 (SEQ ID NO: 656) | 1 | 77 |

Segment cluster AA583399_PEA_{—}1_node_{—}27 (SEQ ID NO:678) according to the present invention is supported by 4 libraries. The number of libraries was determined as previously described. This segment can be found in the following transcript(s): AA583399_PEA_{—}1_T11 (SEQ ID NO:654), AA583399_PEA_{—}1_T12 (SEQ ID NO:655) and AA583399_PEA_{—}1_T15 (SEQ ID NO:656). Table 44 below describes the starting and ending position of this segment on each transcript.

TABLE 44
Segment location on transcripts

| | Segment | Segment |
|---|---|---|
| | starting | ending |
| Transcript name | position | position |
| AA583399_PEA_1_T11 (SEQ ID NO: 654) | 758 | 831 |
| AA583399_PEA_1_T12 (SEQ ID NO: 655) | 1473 | 1546 |
| AA583399_PEA_1_T15 (SEQ ID NO: 656) | 371 | 444 |

### Variant Protein Alignment to the Previously Known Protein:

Sequence name: MYEO_HUMAN_V1 (SEQ ID NO:680)
Sequence documentation:
Alignment of: AA583399_PEA_1_P2 (SEQ ID NO:684) × MYEO_HUMAN_V1 (SEQ ID
NO:680) ..
Alignment segment 1/1:

| | | | |
|---|---|---|---|
| Quality: | 2473.00 | Escore: | 0 |
| Matching length: | 255 | Total length: | 255 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

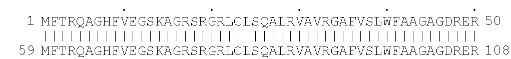

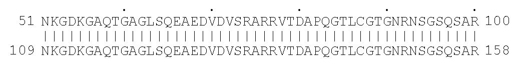

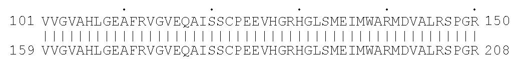

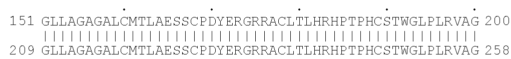

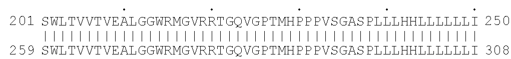

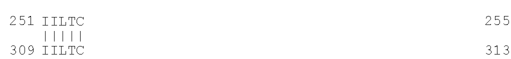

Sequence name: MYEO_HUMAN_V1 (SEQ ID NO:680)
Sequence documentation:
Alignment of: AA583399_PEA_1_P4 (SEQ ID NO:685) × MYEO_HUMAN_V1 (SEQ ID
NO:680) ..
Alignment segment 1/1:

| | | | |
|---|---|---|---|
| Quality: | 1607.00 | Escore: | 0 |
| Matching length: | 164 | Total length: | 164 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

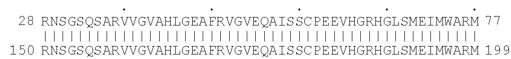

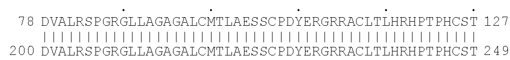

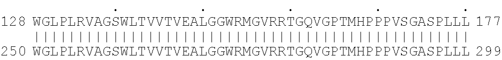

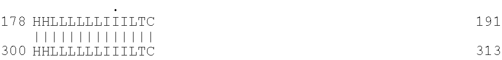

Sequence name: MYEO_HUMAN_V2 (SEQ ID NO:681)
Sequence documentation:
Alignment of: AA583399_PEA_1_P5 (SEQ ID NO:686) × MYEO_HUMAN_V2 (SEQ ID
NO:681) ..
Alignment segment 1/1:

| | | | |
|---|---|---|---|
| Quality: | 1206.00 | Escore: | 0 |
| Matching length: | 122 | Total length: | 122 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

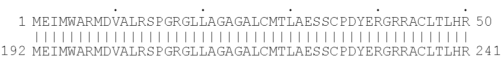

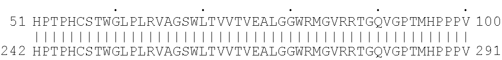

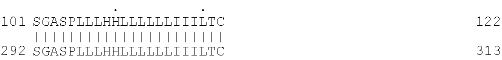

Sequence name: MYEO_HUMAN_V3 (SEQ ID NO:682)
Sequence documentation:
Alignment of: AA583399_PEA_1_P10 (SEQ ID NO:689) × MYEO_HUMAN_V3
(SEQ ID NO:682) ..
Alignment segment 1/1:

| | | | |
|---|---|---|---|
| Quality: | 2475.00 | Escore: | 0 |
| Matching length: | 255 | Total length: | 255 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

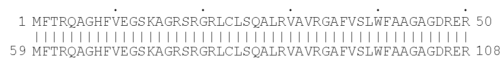

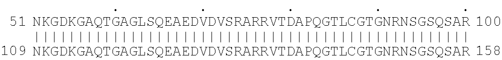

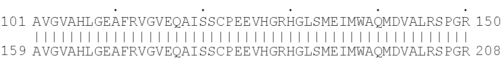

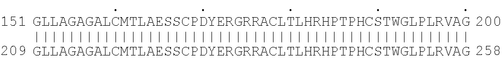

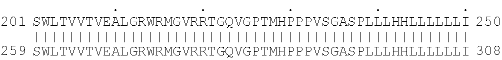

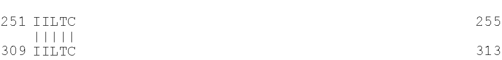

Expression of Myeloma Overexpressed Gene (in a Subset of t(11;14) Positive Multiple Myelomas) (MYEOV) AA583399 Transcripts which are Detectable by Amplicon as Depicted in Sequence Name

### AA583399seg30-32 (SEQ ID NO:1321) in Normal and Cancerous Colon Tissues

Expression of myeloma overexpressed gene (in a subset of t(11;14) positive multiple myelomas) (MYEOV) transcripts detectable by or according to seg30-32, AA583399seg30-32 amplicon (SEQ ID NO:1321) and AA583399seg30-32F (SEQ ID NO: 1319) and AA583399seg30-32R(SEQ ID NO: 1320) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

FIG. 41 is a histogram showing over expression of the above-indicated myeloma overexpressed gene (in a subset of t(11;14) positive multiple myelomas) (MYEOV) transcripts in cancerous colon samples relative to the normal samples. (Values represent the average of duplicate experiments. Error bars indicate the minimal and maximal values obtained.) The number and percentage of samples that exhibit at least 5 fold over-expression, out of the total number of samples tested is indicated in the bottom.

As is evident from FIG. 41, the expression of myeloma overexpressed gene (in a subset of t(11;14) positive multiple myelomas) (MYEOV) transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 5 fold was found in 27 out of 37 adenocarcinoma samples,

Statistical analysis was applied to verify the significance of these results, as described below.

The P value for the difference in the expression levels of myeloma overexpressed gene (in a subset of t(11;14) positive multiple myelomas) (MYEOV) transcripts detectable by the above amplicon in colon cancer samples versus the normal tissue samples was determined by T test as 6.50E-05.

Threshold of 5 fold overexpression was found to differentiate between cancer and normal samples with P value of 1.56E-05 as checked by exact fisher test. The above values demonstrate statistical significance of the results. Primer pairs are also optionally and preferably encompassed within the present invention; for example, for the above experiment, the following primer pair was used as a non-limiting illustrative example only of a suitable primer pair: AA583399seg30-23F forward primer (SEQ ID NO: 1319); and AA583399seg30-32 R reverse primer (SEQ ID NO: 1320). The present invention also preferably encompasses any amplicon obtained through the use of any suitable primer pair; for example, for the above experiment, the following amplicon was obtained as a non-limiting illustrative example only of a suitable amplicon: AA583399seg30-32 (SEQ ID NO: 1321). Forward primer

Forward primer (SEQ ID NO: 1319):
TGGAGATTCCTGGTTTAAAGCATT
Reverse primer (SEQ ID NO: 1320):
CCCCAGCTTAGAGCTGCACT
Amplicon (SEQ ID NO: 1321):
TGGAGATTCCTGGTTTAAAGCATTTAAAGCCTCTGTGAAAATTTGCCCAG
GCCAACAACTTCACTTTCCACACTCAGTGCCACGAAGTGCAGCTCTAAGC
TGGGG

Expression of Myeloma Overexpressed Gene (in a Subset of t(11;14) Positive Multiple Myelomas) (MYEOV) AA583399 Transcripts which are Detectable by Amplicon as Depicted in Sequence Name

### AA583399seg17 (SEQ ID NO: 1324) in Normal and Cancerous Colon Tissues

Expression of myeloma overexpressed gene (in a subset of t(111;14) positive multiple myelomas) (MYEOV) transcripts detectable by or according to seg17, AA583399seg17 amplicon (SEQ ID NO: 1324) and AA583399seg17F (SEQ ID NO: 1322) AA583399seg17R (SEQ ID NO: 1323) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

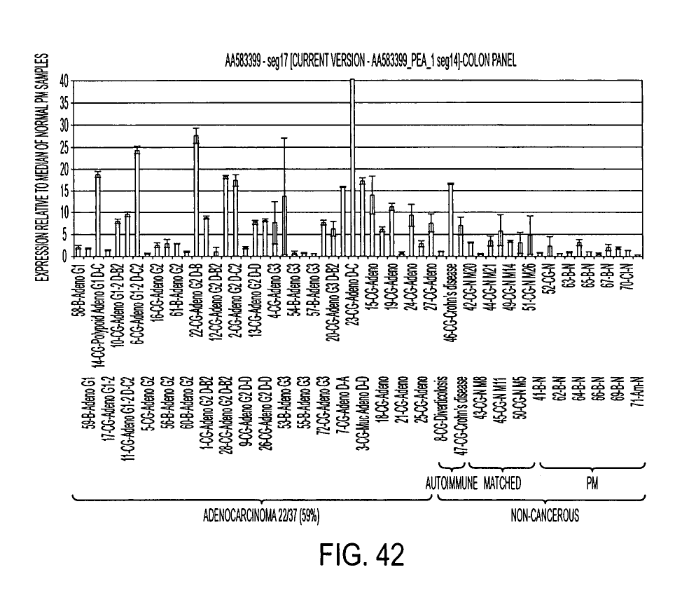

FIG. 42 is a histogram showing over expression of the above-indicated myeloma overexpressed gene (in a subset of t(11;14) positive multiple myelomas) (MYEOV) transcripts in cancerous colon samples relative to the normal samples. (Values represent the average of duplicate experiments. Error bars indicate the minimal and maximal values obtained.) The number and percentage of samples that exhibit at least 5 fold over-expression, out of the total number of samples tested is indicated in the bottom.

As is evident from FIG. 42, the expression of myeloma overexpressed gene (in a subset of t(11;14) positive multiple myelomas) (MYEOV) transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 5 fold was found in 22 out of 37 adenocarcinoma samples.

Statistical analysis was applied to verify the significance of these results, as described below.

The P value for the difference in the expression levels of myeloma overexpressed gene (in a subset of t(11;14) positive multiple myelomas) (MYEOV) transcripts detectable by the above amplicon in colon cancer samples versus the normal tissue samples was determined by T test as 2.37E-04.

Threshold of 5 fold overexpression was found to differentiate between cancer and normal samples with P value of 3.42E-04 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: AA583399seg17F forward primer (SEQ ID NO: 1322); and AA583399seg17 R reverse primer (SEQ ID NO: 1323).

The present invention also preferably encompasses any amplicon obtained through the use of any suitable primer pair; for example, for the above experiment, the following amplicon was obtained as a non-limiting illustrative example only of a suitable amplicon: AA583399seg17 (SEQ ID NO: 1324).

Forward primer (SEQ ID NO: 1322):
CATTCTCCACGCATCAGATGA
Reverse primer (SEQ ID NO: 1323):
ACCATCAGATTGGCAGCATG
Amplicon (SEQ ID NO: 1324):
CATTCTCCACGCATCAGATGATCCTGTGGCCCCTCAGTGCCAGGCCCCAC
TGGCCCTCTGCGCACATCAGTGACTCTGATGTTCTCCCCCACCGCATGCT
GCCAATCTGATGGT

### Expression of Myeloma Overexpressed Gene (in a Subset of t(11;14) Positive Multiple Myelomas) (MYEOV) AA583399 Transcripts which are Detectable by Amplicon as Depicted in Sequence Name AA583399Seg1 (SEQ ID NO: 1327) in Normal and Cancerous Colon Tissues

Expression of myeloma overexpressed gene (in a subset of t(11;14) positive multiple myelomas) (MYEOV) transcripts detectable by or according to seg1, AA583399seg1 amplicon (SEQ ID NO: 1327) and AA583399seg1F (SEQ ID NO: 1325) AA583399seg1R (SEQ ID NO: 1326) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

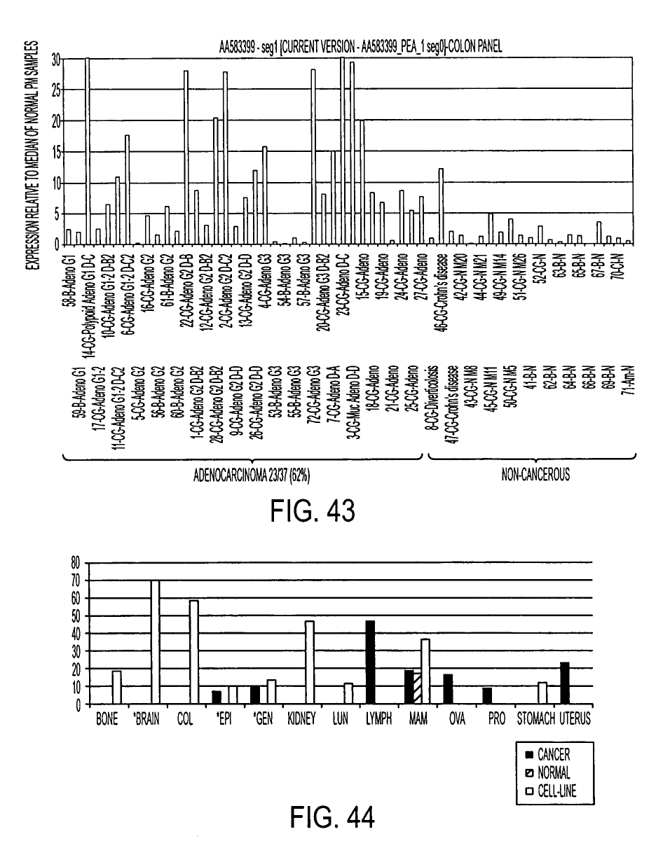

FIG. 43 is a histogram showing over expression of the above-indicated myeloma overexpressed gene (in a subset of t(11;14) positive multiple myelomas) (MYEOV) transcripts in cancerous colon samples relative to the normal samples. The number and percentage of samples that exhibit at least 5 fold over-expression, out of the total number of samples tested is indicated in the bottom.

As is evident from FIG. 43, the expression of myeloma overexpressed gene (in a subset of t(11;14) positive multiple myelomas) (MYEOV) transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 5 fold was found in 23 out of 37 adenocarcinoma samples.

Statistical analysis was applied to verify the significance of these results, as described below.

The P value for the difference in the expression levels of myeloma overexpressed gene (in a subset of t(11;14) positive multiple myelomas) (MYEOV) transcripts detectable by the above amplicon in colon cancer samples versus the normal tissue samples was determined by T test as 1.55E-05.

Threshold of 5 fold overexpression was found to differentiate between cancer and normal samples with P value of 1.97E-04 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: AA583399seg1F forward primer (SEQ ID NO: 1325); and AA583399seg1 R reverse primer (SEQ ID NO: 1326).

The present invention also preferably encompasses any amplicon obtained through the use of any suitable primer pair; for example, for the above experiment, the following amplicon was obtained as a non-limiting illustrative example only of a suitable amplicon: AA583399seg1 (SEQ ID NO: 1327).

Forward primer (SEQ ID NO: 1325):
GAATCAGCCCAAAGCCAGG
Reverse primer (SEQ ID NO: 1326):
GCTGGTGAAAGCACTGGGTT
Amplicon (SEQ ID NO: 1327):
GAATCAGCCCAAAGCCAGGCGTCCAGGGTCTCCCTCACCTGAAGCTGACT
TTTTCCCCACCTTGGACAGAGGGCGGGAGATGCCATCCCCACTGAACCCA
GTGCTTTCACCAGC

### Description for Cluster AI684092

Cluster AI684092 features 2 transcript(s) and 8 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| AI684092_PEA_1_T2 | 693 |
| AI684092_PEA_1_T3 | 694 |

TABLE 2
Segments of interest

| Segment Name | SEQ ID NO: |
|---|---|
| AI684092_PEA_1_node_0 | 695 |
| AI684092_PEA_1_node_2 | 696 |
| AI684092_PEA_1_node_4 | 697 |
| AI684092_PEA_1_node_5 | 698 |
| AI684092_PEA_1_node_6 | 699 |
| AI684092_PEA_1_node_7 | 700 |
| AI684092_PEA_1_node_8 | 701 |
| AI684092_PEA_1_node_9 | 702 |

TABLE 3
Proteins of interest

| Protein Name | SEQ ID NO: | Corresponding Transcript(s) |
|---|---|---|
| AI684092_PEA_1_P1 | 703 | AI684092_PEA_1_T2 |
| | | (SEQ ID NO: 693) |
| AI684092_PEA_1_P3 | 704 | AI684092_PEA_1_T3 |
| | | (SEQ ID NO: 694) |

Cluster AI684092 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 44 and Table 4. This cluster is overexpressed (at least at a minimum level) in the following pathological conditions: brain malignant tumors, epithelial malignant tumors and a mixture of malignant tumors from different tissues.

TABLE 4
Normal tissue distribution

| Name of Tissue | Number |
|---|---|
| bone | 0 |
| brain | 0 |
| colon | 0 |
| epithelial | 0 |
| general | 0 |
| kidney | 0 |
| lung | 0 |
| lymph nodes | 0 |
| breast | 17 |
| ovary | 0 |
| prostate | 0 |
| stomach | 0 |
| uterus | 0 |

TABLE 5
P values and ratios for expression in cancerous tissue

| Name of Tissue | P1 | P2 | SP1 | R3 | SP2 | R4 |
|---|---|---|---|---|---|---|
| bone | 1 | 6.7e−01 | 1 | 1.0 | 7.0e−01 | 1.4 |
| brain | 1 | 1.3e−01 | 1 | 1.0 | 3.8e−03 | 9.2 |
| colon | 1 | 4.8e−01 | 1 | 1.0 | 5.9e−01 | 1.6 |
| epithelial | 7.2e−02 | 2.0e−02 | 4.1e−02 | 3.9 | 8.9e−03 | 4.8 |
| general | 3.0e−03 | 2.0e−04 | 1.4e−04 | 8.1 | 1.2e−06 | 9.0 |
| kidney | 1 | 7.2e−01 | 1 | 1.0 | 4.9e−01 | 1.9 |
| lung | 1 | 6.3e−01 | 1 | 1.0 | 6.2e−01 | 1.6 |
| lymph nodes | 3.1e−01 | 5.7e−01 | 2.9e−01 | 3.5 | 5.8e−01 | 1.7 |
| breast | 8.2e−01 | 7.3e−01 | 6.9e−01 | 1.0 | 5.6e−01 | 1.2 |
| ovary | 6.2e−01 | 6.5e−01 | 6.8e−01 | 1.5 | 7.7e−01 | 1.3 |
| prostate | 7.3e−01 | 7.8e−01 | 6.7e−01 | 1.5 | 7.5e−01 | 1.3 |
| stomach | 3.6e−01 | 4.7e−01 | 1 | 1.0 | 8.0e−01 | 1.3 |
| uterus | 2.1e−01 | 4.0e−01 | 4.4e−01 | 2.0 | 6.4e−01 | 1.5 |

As noted above, cluster AI684092 features 2 transcript(s), which were listed in Table 1 above. A description of each variant protein according to the present invention is now provided.

Variant protein AI684092_PEA_{—}1_P1 (SEQ ID NO:703) according to the present invention has an amino acid sequence as given at the end of the application; it is encoded by transcript(s) AI684092_PEA_{—}1_T2 (SEQ ID NO:693). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein AI684092_PEA_{—}1_P1 (SEQ ID NO:703) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein AI684092_PEA_{—}1_P1 (SEQ ID NO:703) sequence provides support for the deduced sequence of this variant protein according to the present invention).

TABLE 6
Amino acid mutations

| SNP position(s) on amino acid | | Previously |
|---|---|---|
| sequence | Alternative amino acid(s) | known SNP? |
| 137 | C -> Y | Yes |
| 160 | S -> P | Yes |
| 164 | A -> D | Yes |
| 168 | S -> R | Yes |
| 175 | A -> D | Yes |

Variant protein AI684092_PEA_{—}1_P1 (SEQ ID NO:703) is encoded by the following transcript(s): AI684092_PEA_{—}1_T2 (SEQ ID NO:693), for which the sequence(s) is/are given at the end of the application. The coding portion of transcript AI684092_PEA_{—}1_T2 (SEQ ID NO:693) is shown in bold; this coding portion starts at position 1480 and ends at position 2058. The transcript also has the following SNPs as listed in Table 7 (given according to their position on the nucleotide sequence, with the alternative nucleic acid listed; the last column indicates whether the SNP is known or not; the presence of known SNPs in variant protein AI684092_PEA_{—}1_P1 (SEQ ID NO:703) sequence provides support for the deduced sequence of this variant protein according to the present invention).

TABLE 7
Nucleic acid SNPs

| SNP position on nucleotide | | Previously |
|---|---|---|
| sequence | Alternative nucleic acid | known SNP? |
| 609 | G -> A | Yes |
| 807 | T -> C | Yes |
| 1284 | T -> C | No |
| 1295 | T -> G | No |
| 1889 | G -> A | Yes |
| 1932 | C -> G | Yes |
| 1941 | C -> T | Yes |
| 1957 | T -> C | Yes |
| 1970 | C -> A | Yes |
| 1983 | T -> G | Yes |
| 2003 | C -> A | Yes |
| 2019 | C -> G | Yes |
| 2052 | G -> C | Yes |
| 2142 | A -> T | Yes |

Variant protein AI684092_PEA_{—}1_P3 (SEQ ID NO:704) according to the present invention has an amino acid sequence as given at the end of the application; it is encoded by transcript(s) AI684092_PEA_{—}1_T3 (SEQ ID NO:694). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein AI684092_PEA_{—}1_P3 (SEQ ID NO:704) is encoded by the following transcript(s): AI684092_PEA_{—}1_T3 (SEQ ID NO:694), for which the sequence(s) is/are given at the end of the application. The coding portion of transcript AI684092_PEA_{—}1_T3 (SEQ ID NO:694) is shown in bold; this coding portion starts at position 28 and ends at position 279. The transcript also has the following SNPs as listed in Table 8 (given according to their position on the nucleotide sequence, with the alternative nucleic acid listed; the last column indicates whether the SNP is known or not; the presence of known SNPs in variant protein AI684092_PEA_{—}1_P3 (SEQ ID NO:704) sequence provides support for the deduced sequence of this variant protein according to the present invention).

TABLE 8
Nucleic acid SNPs

| SNP position on nucleotide | | Previously |
|---|---|---|
| sequence | Alternative nucleic acid | known SNP? |
| 609 | G -> A | Yes |
| 807 | T -> C | Yes |
| 1284 | T -> C | No |
| 1295 | T -> G | No |
| 1571 | C -> A | Yes |
| 1584 | T -> G | Yes |
| 1604 | C -> A | Yes |
| 1620 | C -> G | Yes |
| 1653 | G -> C | Yes |
| 1743 | A -> T | Yes |

As noted above, cluster AI684092 features 8 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster AI684092_PEA_{—}1_node_{—}0 (SEQ ID NO:695) according to the present invention is supported by 5 libraries. The number of libraries was determined as previously described. This segment can be found in the following transcript(s): AI684092_PEA_{—}1_T2 (SEQ ID NO:693) and AI684092_PEA_{—}1_T3 (SEQ ID NO:694). Table 9 below describes the starting and ending position of this segment on each transcript.

TABLE 9
Segment location on transcripts

| | Segment | |
|---|---|---|
| | starting | Segment |
| Transcript name | position | ending position |
| AI684092_PEA_1_T2 (SEQ ID NO: 693) | 1 | 368 |
| AI684092_PEA_1_T3 (SEQ ID NO: 694) | 1 | 368 |

Segment cluster AI684092_PEA_{—}1_node_{—}2 (SEQ ID NO:696) according to the present invention is supported by 5 libraries. The number of libraries was determined as previously described. This segment can be found in the following transcript(s): AI684092_PEA_{—}1_T2 (SEQ ID NO:693) and AI684092_PEA_{—}1_T3 (SEQ ID NO:694). Table 10 below describes the starting and ending position of this segment on each transcript.

TABLE 10
Segment location on transcripts

| | Segment | |
|---|---|---|
| | starting | Segment |
| Transcript name | position | ending position |
| AI684092_PEA_1_T2 (SEQ ID NO: 693) | 369 | 498 |
| AI684092_PEA_1_T3 (SEQ ID NO: 694) | 369 | 498 |

Segment cluster AI684092_PEA_{—}1_node_{—}4 (SEQ ID NO:697) according to the present invention is supported by 7 libraries. The number of libraries was determined as previously described. This segment can be found in the following transcript(s): AI684092_PEA_{—}1_T2 (SEQ ID NO:693) and AI684092_PEA_{—}1_T3 (SEQ ID NO:694). Table 11 below describes the starting and ending position of this segment on each transcript.

TABLE 11
Segment location on transcripts

| | Segment | Segment |
|---|---|---|
| | starting | ending |
| Transcript name | position | position |
| AI684092_PEA_1_T2 (SEQ ID NO: 693) | 499 | 665 |
| AI684092_PEA_1_T3 (SEQ ID NO: 694) | 499 | 665 |

Segment cluster AI684092_PEA_{—}1_node_{—}5 (SEQ ID NO:698) according to the present invention is supported by 7 libraries. The number of libraries was determined as previously described. This segment can be found in the following transcript(s): AI684092_PEA_{—}1_T2 (SEQ ID NO:693) and AI684092_PEA_{—}1_T3 (SEQ ID NO:694). Table 12 below describes the starting and ending position of this segment on each transcript.

TABLE 12
Segment location on transcripts

| | Segment | Segment |
|---|---|---|
| | starting | ending |
| Transcript name | position | position |
| AI684092_PEA_1_T2 (SEQ ID NO: 693) | 666 | 932 |
| AI684092_PEA_1_T3 (SEQ ID NO: 694) | 666 | 932 |

Segment cluster AI684092_PEA_{—}1_node_{—}6 (SEQ ID NO:699) according to the present invention is supported by 7 libraries. The number of libraries was determined as previously described. This segment can be found in the following transcript(s): AI684092_PEA_{—}1_T2 (SEQ ID NO:693) and AI684092_PEA_{—}1_T3 (SEQ ID NO:694). Table 13 below describes the starting and ending position of this segment on each transcript.

TABLE 13
Segment location on transcripts

| | Segment | Segment |
|---|---|---|
| | starting | ending |
| Transcript name | position | position |
| AI684092_PEA_1_T2 (SEQ ID NO: 693) | 933 | 1372 |
| AI684092_PEA_1_T3 (SEQ ID NO: 694) | 933 | 1372 |

Segment cluster AI684092_PEA_{—}1_node_{—}7 (SEQ ID NO:700) according to the present invention is supported by 9 libraries. The number of libraries was determined as previously described. This segment can be found in the following transcript(s): AI684092_PEA_{—}1_T2 (SEQ ID NO:693) and AI684092_PEA_{—}1_T3 (SEQ ID NO:694). Table 14 below describes the starting and ending position of this segment on each transcript.

TABLE 14
Segment location on transcripts

| | Segment | Segment |
|---|---|---|
| | starting | ending |
| Transcript name | position | position |
| AI684092_PEA_1_T2 (SEQ ID NO: 693) | 1373 | 1560 |
| AI684092_PEA_1_T3 (SEQ ID NO: 694) | 1373 | 1560 |

Segment cluster AI684092_PEA_{—}1_node_{—}8 (SEQ ID NO:701) according to the present invention is supported by 10 libraries. The number of libraries was determined as previously described. This segment can be found in the following transcript(s): AI684092_PEA_{—}1_T2 (SEQ ID NO:693). Table 15 below describes the starting and ending position of this segment on each transcript.

TABLE 15
Segment location on transcripts

| | Segment | Segment |
|---|---|---|
| | starting | ending |
| Transcript name | position | position |
| AI684092_PEA_1_T2 (SEQ ID NO: 693) | 1561 | 1959 |

Segment cluster AI684092_PEA_{—}1_node_{—}9 (SEQ ID NO:702) according to the present invention is supported by 9 libraries. The number of libraries was determined as previously described. This segment can be found in the following transcript(s): AI684092_PEA_{—}1_T2 (SEQ ID NO:693) and AI684092_PEA_{—}1_T3 (SEQ ID NO:694). Table 16 below describes the starting and ending position of this segment on each transcript.

TABLE 16
Segment location on transcripts

| | Segment | Segment |
|---|---|---|
| | starting | ending |
| Transcript name | position | position |
| AI684092_PEA_1_T2 (SEQ ID NO: 693) | 1960 | 2195 |
| AI684092_PEA_1_T3 (SEQ ID NO: 694) | 1561 | 1796 |

### Example 1

### Expression of AA5315457 Transcripts which are Detectable by Amplicon as Depicted in Sequence Name AA5315457seg8 (SEQ ID NO: 1330) in Normal and Cancerous Colon Tissues

Expression of AA5315457 transcripts detectable by or according to seg8, AA5315457 seg8 amplicon (SEQ ID NO: 1330) and AA5315457F (SEQ ID NO: 1328) AA5315457R (SEQ ID NO: 1329) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

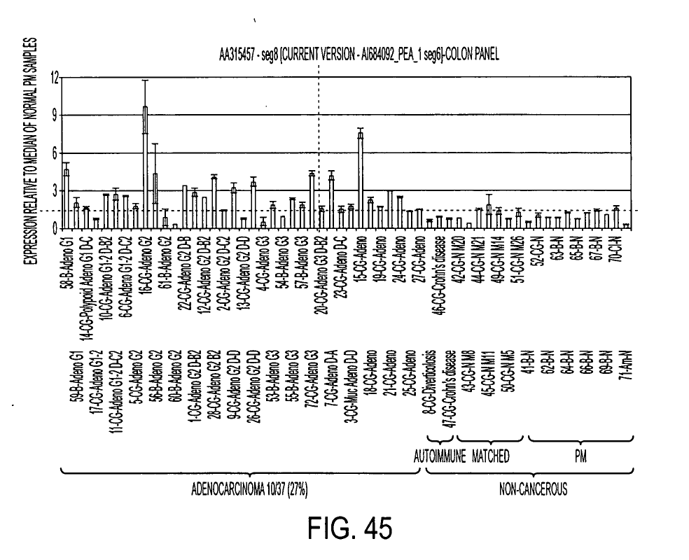

FIG. 45 is a histogram showing over expression of the above-indicated AA5315457 transcripts in cancerous colon samples relative to the normal samples. (Values represent the average of duplicate experiments. Error bars indicate the minimal and maximal values obtained.) The number and percentage of samples that exhibit at least 3 fold over-expression, out of the total number of samples tested is indicated in the bottom.

As is evident from FIG. 45, the expression of AA5315457 transcripts detectable by the above amplicon in cancer samples was significantly higher than in the non-cancerous samples (Sample Nos. 41, 52, 62-67, 69-71, Table 1 above, “Tissue samples in testing panel”). Notably an over-expression of at least 3 fold was found in 10 out of 37 adenocarcinoma samples,

Statistical analysis was applied to verify the significance of these results, as described below.

The P value for the difference in the expression levels of AA5315457 transcripts detectable by the above amplicon in colon cancer samples versus the normal tissue samples was determined by T test as 1.66E-05.

Threshold of 3 fold overexpression was found to differentiate between cancer and normal samples with P value of 5.33E-02 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: AA5315457F forward primer (SEQ ID NO: 1328); and AA5315457R reverse primer (SEQ ID NO: 1329).

The present invention also preferably encompasses any amplicon obtained through the use of any suitable primer pair; for example, for the above experiment, the following amplicon was obtained as a non-limiting illustrative example only of a suitable amplicon: AA5315457.

Forward primer (SEQ ID NO: 1328):
CATGGACCCCAGGCAAGTC
Reversr primer (SEQ ID NO: 1329):
CTGTTTAGGGTCGAGGCTGTG
Amplicon (SEQ ID NO: 1330):
CATGGACCCCAGGCAAGTCCCCCCACCCACGCATTTCTAATCATCTGCCC
TGGTTTTGCCTCCTGAGTCTGTTAAGGCTGTGTGCCCCTCATCGAGGCCC
GTCACAGCCTCGACCCTAAACAG

### Description for Cluster HUMCACH1A

Cluster HUMCACH1A features 18 transcript(s) and 67 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| HUMCACH1A_PEA_1_T0 | 705 |
| HUMCACH1A_PEA_1_T1 | 706 |
| HUMCACH1A_PEA_1_T2 | 707 |
| HUMCACH1A_PEA_1_T3 | 708 |
| HUMCACH1A_PEA_1_T4 | 709 |
| HUMCACH1A_PEA_1_T6 | 710 |
| HUMCACH1A_PEA_1_T7 | 711 |
| HUMCACH1A_PEA_1_T8 | 712 |
| HUMCACH1A_PEA_1_T12 | 713 |
| HUMCACH1A_PEA_1_T13 | 714 |
| HUMCACH1A_PEA_1_T14 | 715 |
| HUMCACH1A_PEA_1_T15 | 716 |
| HUMCACH1A_PEA_1_T16 | 717 |
| HUMCACH1A_PEA_1_T17 | 718 |
| HUMCACH1A_PEA_1_T18 | 719 |
| HUMCACH1A_PEA_1_T19 | 720 |
| HUMCACH1A_PEA_1_T20 | 721 |
| HUMCACH1A_PEA_1_T22 | 722 |

TABLE 2
Segments of interest

| Segment Name | SEQ ID NO: |
|---|---|
| HUMCACH1A_PEA_1_node_2 | 723 |
| HUMCACH1A_PEA_1_node_5 | 724 |
| HUMCACH1A_PEA_1_node_9 | 725 |
| HUMCACH1A_PEA_1_node_11 | 726 |
| HUMCACH1A_PEA_1_node_14 | 727 |
| HUMCACH1A_PEA_1_node_16 | 728 |
| HUMCACH1A_PEA_1_node_27 | 729 |
| HUMCACH1A_PEA_1_node_30 | 730 |
| HUMCACH1A_PEA_1_node_33 | 731 |
| HUMCACH1A_PEA_1_node_41 | 732 |
| HUMCACH1A_PEA_1_node_43 | 733 |
| HUMCACH1A_PEA_1_node_45 | 734 |
| HUMCACH1A_PEA_1_node_47 | 735 |
| HUMCACH1A_PEA_1_node_55 | 736 |
| HUMCACH1A_PEA_1_node_57 | 737 |
| HUMCACH1A_PEA_1_node_70 | 738 |
| HUMCACH1A_PEA_1_node_72 | 739 |
| HUMCACH1A_PEA_1_node_74 | 740 |
| HUMCACH1A_PEA_1_node_86 | 741 |
| HUMCACH1A_PEA_1_node_92 | 742 |
| HUMCACH1A_PEA_1_node_94 | 743 |
| HUMCACH1A_PEA_1_node_103 | 744 |
| HUMCACH1A_PEA_1_node_104 | 745 |
| HUMCACH1A_PEA_1_node_106 | 746 |
| HUMCACH1A_PEA_1_node_109 | 747 |
| HUMCACH1A_PEA_1_node_113 | 748 |
| HUMCACH1A_PEA_1_node_114 | 749 |
| HUMCACH1A_PEA_1_node_116 | 750 |
| HUMCACH1A_PEA_1_node_119 | 751 |
| HUMCACH1A_PEA_1_node_121 | 752 |
| HUMCACH1A_PEA_1_node_123 | 753 |
| HUMCACH1A_PEA_1_node_125 | 754 |
| HUMCACH1A_PEA_1_node_128 | 755 |
| HUMCACH1A_PEA_1_node_0 | 756 |
| HUMCACH1A_PEA_1_node_3 | 757 |
| HUMCACH1A_PEA_1_node_7 | 758 |
| HUMCACH1A_PEA_1_node_23 | 759 |
| HUMCACH1A_PEA_1_node_26 | 760 |
| HUMCACH1A_PEA_1_node_32 | 761 |
| HUMCACH1A_PEA_1_node_35 | 762 |
| HUMCACH1A_PEA_1_node_37 | 763 |
| HUMCACH1A_PEA_1_node_39 | 764 |
| HUMCACH1A_PEA_1_node_49 | 765 |
| HUMCACH1A_PEA_1_node_51 | 766 |
| HUMCACH1A_PEA_1_node_53 | 767 |
| HUMCACH1A_PEA_1_node_58 | 768 |
| HUMCACH1A_PEA_1_node_60 | 769 |
| HUMCACH1A_PEA_1_node_62 | 770 |
| HUMCACH1A_PEA_1_node_64 | 771 |
| HUMCACH1A_PEA_1_node_66 | 772 |
| HUMCACH1A_PEA_1_node_68 | 773 |
| HUMCACH1A_PEA_1_node_76 | 774 |
| HUMCACH1A_PEA_1_node_77 | 775 |
| HUMCACH1A_PEA_1_node_79 | 776 |
| HUMCACH1A_PEA_1_node_81 | 777 |
| HUMCACH1A_PEA_1_node_84 | 778 |
| HUMCACH1A_PEA_1_node_88 | 779 |
| HUMCACH1A_PEA_1_node_90 | 780 |
| HUMCACH1A_PEA_1_node_96 | 781 |
| HUMCACH1A_PEA_1_node_98 | 782 |
| HUMCACH1A_PEA_1_node_100 | 783 |
| HUMCACH1A_PEA_1_node_101 | 784 |
| HUMCACH1A_PEA_1_node_107 | 785 |
| HUMCACH1A_PEA_1_node_111 | 786 |
| HUMCACH1A_PEA_1_node_117 | 787 |
| HUMCACH1A_PEA_1_node_124 | 788 |
| HUMCACH1A_PEA_1_node_126 | 789 |

TABLE 3
Proteins of interest

| Protein Name | SEQ ID NO: | Corresponding Transcript(s) |
|---|---|---|
| HUMCACH1A_PEA_1_P2 | 792 | HUMCACH1A_PEA_1_T0 (SEQ ID NO: 705); |
| | | HUMCACH1A_PEA_1_T1 (SEQ ID NO: 706); |
| | | HUMCACH1A_PEA_1_T2 (SEQ ID NO: 707); |
| | | HUMCACH1A_PEA_1_T3 (SEQ ID NO: 708); |
| | | HUMCACH1A_PEA_1_T4 (SEQ ID NO: 709) |
| HUMCACH1A_PEA_1_P3 | 793 | HUMCACH1A_PEA_1_T6 (SEQ ID NO: 710) |
| HUMCACH1A_PEA_1_P4 | 794 | HUMCACH1A_PEA_1_T7 (SEQ ID NO: 711) |
| HUMCACH1A_PEA_1_P5 | 795 | HUMCACH1A_PEA_1_T8 (SEQ ID NO: 712) |
| HUMCACH1A_PEA_1_P7 | 796 | HUMCACH1A_PEA_1_T12 (SEQ ID NO: 713) |
| HUMCACH1A_PEA_1_P8 | 797 | HUMCACH1A_PEA_1_T13 (SEQ ID NO: 714) |
| HUMCACH1A_PEA_1_P9 | 798 | HUMCACH1A_PEA_1_T14 (SEQ ID NO: 715) |
| HUMCACH1A_PEA_1_P10 | 799 | HUMCACH1A_PEA_1_T15 (SEQ ID NO: 716) |
| HUMCACH1A_PEA_1_P11 | 800 | HUMCACH1A_PEA_1_T16 (SEQ ID NO: 717) |
| HUMCACH1A_PEA_1_P12 | 801 | HUMCACH1A_PEA_1_T17 (SEQ ID NO: 718) |
| HUMCACH1A_PEA_1_P13 | 802 | HUMCACH1A_PEA_1_T18 (SEQ ID NO: 719) |
| HUMCACH1A_PEA_1_P14 | 803 | HUMCACH1A_PEA_1_T19 (SEQ ID NO: 720) |
| HUMCACH1A_PEA_1_P15 | 804 | HUMCACH1A_PEA_1_T20 (SEQ ID NO: 721) |
| HUMCACH1A_PEA_1_P17 | 805 | HUMCACH1A_PEA_1_T22 (SEQ ID NO: 722) |

These sequences are variants of the known protein Voltage-dependent L-type calcium channel alpha-1D subunit (SwissProt accession identifier CCAD_HUMAN; known also according to the synonyms Calcium channel, L type, alpha-1 polypeptide, isoform 2), SEQ ID NO: 790, referred to herein as the previously known protein.

Protein Voltage-dependent L-type calcium channel alpha-1D subunit (SEQ ID NO:790) is known or believed to have the following function(s): Voltage-sensitive calcium channels (VSCC) mediate the entry of calcium ions into excitable cells and are also involved in a variety of calcium-dependent processes, including muscle contraction, hormone or neurotransmitter release, gene expression, cell motility, cell division and cell death. The isoform alpha-1D gives rise to L-type calcium currents. Long-lasting (L-type) calcium channels belong to the “high-voltage activated” (HVA) group. They are blocked by dihydropyridines (DHP), phenylalkylamines, benzothiazepines, and by omega-agatoxin-IIIA (omega-aga-IIIA). They are however insensitive to omega-conotoxin-GVIA (omega-CTx-GVIA) and omega-agatoxin-IVA (omega-aga-IVA). The sequence for protein Voltage-dependent L-type calcium channel alpha-1D subunit (SEQ ID NO:790) is given at the end of the application, as “Voltage-dependent L-type calcium channel alpha-1D subunit (SEQ ID NO:790) amino acid sequence”. Known polymorphisms for this sequence are as shown in Table 4.

TABLE 4
Amino acid mutations for Known Protein

| SNP position(s) on amino | |
|---|---|
| acid sequence | Comment |
| 1 | M -> MM (in a NIDDM patient). |
| | /FTId = VAR_001497. |
| 576 | S -> T |
| 637 | C -> S |
| 650 | S -> I |
| 918 | I -> T |
| 960 | M -> I |
| 1289-1290 | Missing |
| 1346 | F -> S |
| 1433 | H -> Y |

Protein Voltage-dependent L-type calcium channel alpha-1D subunit (SEQ ID NO:790) localization is believed to be Integral membrane protein.

The following GO Annotation(s) apply to the previously known protein. The following annotation(s) were found: transport; cation transport; calcium ion transport, which are annotation(s) related to Biological Process; calcium binding; dihydropyridine-sensitive calcium channel, which are annotation(s) related to Molecular Function; and voltage-gated calcium channel; integral membrane protein, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster HUMCACH1A can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

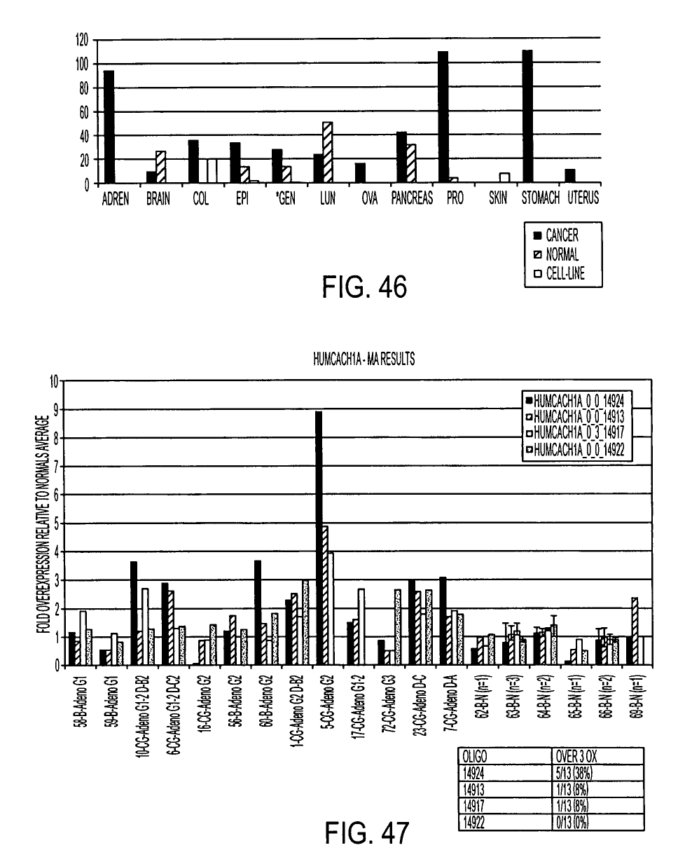

Overall, the following results were obtained as shown with regard to the histograms in FIG. 46 and Table 5. This cluster is overexpressed (at least at a minimum level) in the following pathological conditions: a mixture of malignant tumors from different tissues.

TABLE 5
Normal tissue distribution

| Name of Tissue | Number |
|---|---|
| adrenal | 0 |
| brain | 27 |
| colon | 0 |
| epithelial | 13 |
| general | 13 |
| lung | 51 |
| ovary | 0 |
| pancreas | 32 |
| prostate | 4 |
| skin | 0 |
| stomach | 0 |
| uterus | 0 |

TABLE 6
P values and ratios for expression in cancerous tissue

| Name of Tissue | P1 | P2 | SP1 | R3 | SP2 | R4 |
|---|---|---|---|---|---|---|
| adrenal | 6.1e−01 | 6.6e−01 | 2.1e−01 | 3.4 | 2.9e−01 | 2.7 |
| brain | 8.7e−01 | 8.9e−01 | 9.2e−01 | 0.4 | 9.8e−01 | 0.3 |
| colon | 1.1e−01 | 1.1e−01 | 4.9e−01 | 2.2 | 4.6e−01 | 2.0 |
| epithelial | 2.6e−02 | 2.2e−01 | 8.8e−03 | 2.4 | 2.5e−01 | 1.3 |
| general | 7.1e−03 | 1.7e−01 | 4.3e−03 | 2.0 | 4.0e−01 | 1.0 |
| lung | 8.7e−01 | 9.2e−01 | 9.6e−01 | 0.6 | 1 | 0.3 |
| ovary | 6.2e−01 | 6.5e−01 | 6.8e−01 | 1.5 | 7.7e−01 | 1.3 |
| pancreas | 4.3e−01 | 6.5e−01 | 5.0e−01 | 1.2 | 7.0e−01 | 0.9 |
| prostate | 3.1e−01 | 4.7e−01 | 1.2e−02 | 4.9 | 4.2e−02 | 3.6 |
| skin | 1 | 4.4e−01 | 1 | 1.0 | 6.4e−01 | 1.6 |
| stomach | 3.0e−01 | 6.7e−01 | 1.3e−01 | 3.0 | 5.1e−01 | 1.5 |
| uterus | 4.7e−01 | 6.4e−01 | 6.6e−01 | 1.5 | 8.0e−01 | 1.2 |

As noted above, cluster HUMCACH1A features 18 transcript(s), which were listed in Table 1 above. These transcript(s) encode for protein(s) which are variant(s) of protein Voltage-dependent L-type calcium channel alpha-1D subunit (SEQ ID NO:790). A description of each variant protein according to the present invention is now provided.

Variant protein HUMCACH1A_PEA_{—}1_P2 (SEQ ID NO:792) according to the present invention has an amino acid sequence as given at the end of the application; it is encoded by transcript(s) HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708) and HUMCACH1A_PEA_{—}1_T4 (SEQ ID NO:709). The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein HUMCACH1A_PEA_{—}1_P2 (SEQ ID NO:792) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein HUMCACH1A_PEA_{—}1_P2 (SEQ ID NO:792) sequence provides support for the deduced sequence of this variant protein according to the present invention).

TABLE 7
Amino acid mutations

| SNP Position(s) on amino acid | Alternative | Previously |
|---|---|---|
| sequence | amino acid(s) | known SNP? |
| 577 | I -> M | Yes |
| 809 | N -> | No |
| 1038 | I -> T | No |

Variant protein HUMCACH1A_PEA_{—}1_P2 (SEQ ID NO:792) is encoded by the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708) and HUMCACH1A_PEA_{—}1_T4 (SEQ ID NO:709), for which the sequence(s) is/are given at the end of the application.

The coding portion of transcript HUMCACH1A_PEA_{—}1_T0 (SEQ ID NO:705) is shown in bold; this coding portion starts at position 512 and ends at position 7054. The transcript also has the following SNPs as listed in Table 8 (given according to their position on the nucleotide sequence, with the alternative nucleic acid listed; the last column indicates whether the SNP is known or not; the presence of known SNPs in variant protein HUMCACH1A_PEA_{—}1_P2 (SEQ ID NO:792) sequence provides support for the deduced sequence of this variant protein according to the present invention).

TABLE 8
Nucleic acid SNPs

| SNP Position on uncleotide | Alternative | Previously |
|---|---|---|
| sequence | nucleic acid | known SNP? |
| 331 | T -> A | Yes |
| 422 | C -> T | Yes |
| 458 | T -> C | Yes |
| 970 | T -> C | Yes |
| 1615 | C -> T | Yes |
| 2242 | T -> G | Yes |
| 2296 | C -> T | No |
| 2381 | T -> C | Yes |
| 2937 | A -> | No |
| 3271 | C -> T | Yes |
| 3624 | T -> C | No |
| 3637 | C -> T | Yes |
| 6919 | C -> T | Yes |
| 7383 | A -> G | Yes |
| 7701 | A -> G | Yes |
| 8070 | C -> T | Yes |
| 8117 | T -> A | Yes |
| 8361 | T -> C | Yes |
| 8544 | G -> A | Yes |
| 8632 | A -> C | Yes |
| 8685 | A -> G | Yes |
| 9028 | G -> A | Yes |
| 9375 | G -> | No |
| 9375 | G -> A | No |

The coding portion of transcript HUMCACH1A_PEA_{—}1_T1 (SEQ ID NO:706) is shown in bold; this coding portion starts at position 89 and ends at position 6631. The transcript also has the following SNPs as listed in Table 9 (given according to their position on the nucleotide sequence, with the alternative nucleic acid listed; the last column indicates whether the SNP is known or not; the presence of known SNPs in variant protein HUMCACH1A_PEA_{—}1_P2 (SEQ ID NO:792) sequence provides support for the deduced sequence of this variant protein according to the present invention).

TABLE 9
Nucleic acid SNPs

| SNP position on nucleotide | Alternative | Previously |
|---|---|---|
| sequence | nucleic acid | known SNP? |
| 547 | T -> C | Yes |
| 1192 | C -> T | Yes |
| 1819 | T -> G | Yes |
| 1873 | C -> T | No |
| 1958 | T -> C | Yes |
| 2514 | A -> | No |
| 2848 | C -> T | Yes |
| 3201 | T -> C | No |
| 3214 | C -> T | Yes |
| 6496 | C -> T | Yes |
| 6960 | A -> G | Yes |
| 7278 | A -> G | Yes |
| 7647 | C -> T | Yes |
| 7694 | T -> A | Yes |
| 7938 | T -> C | Yes |
| 8121 | G -> A | Yes |
| 8209 | A -> C | Yes |
| 8262 | A -> G | Yes |
| 8605 | G -> A | Yes |
| 8952 | G -> | No |
| 8952 | G -> A | No |

The coding portion of transcript HUMCACH1A_PEA_{—}1_T2 (SEQ ID NO:707) is shown in bold; this coding portion starts at position 512 and ends at position 7054. The transcript also has the following SNPs as listed in Table 10 (given according to their position on the nucleotide sequence, with the alternative nucleic acid listed; the last column indicates whether the SNP is known or not; the presence of known SNPs in variant protein HUMCACH1A_PEA_{—}1_P2 (SEQ ID NO:792) sequence provides support for the deduced sequence of this variant protein according to the present invention).

TABLE 10
Nucleic acid SNPs

| SNP position on nucleotide | Alternative | Previously |
|---|---|---|
| sequence | nucleic acid | known SNP? |
| 331 | T -> A | Yes |
| 422 | C -> T | Yes |
| 458 | T -> C | Yes |
| 970 | T -> C | Yes |
| 1615 | C -> T | Yes |
| 2242 | T -> G | Yes |
| 2296 | C -> T | No |
| 2381 | T -> C | Yes |
| 2937 | A -> | No |
| 3271 | C -> T | Yes |
| 3624 | T -> C | No |
| 3637 | C -> T | Yes |
| 6919 | C -> T | Yes |
| 7383 | A -> G | Yes |
| 7701 | A -> G | Yes |
| 8070 | C -> T | Yes |
| 8117 | T -> A | Yes |
| 8361 | T -> C | Yes |
| 8544 | G -> A | Yes |
| 8632 | A -> C | Yes |
| 8685 | A -> G | Yes |
| 9028 | G -> A | Yes |

The coding portion of transcript HUMCACH1A_PEA_{—}1_T3 (SEQ ID NO:708) is shown in bold; this coding portion starts at position 512 and ends at position 7054. The transcript also has the following SNPs as listed in Table 11 (given according to their position on the nucleotide sequence, with the alternative nucleic acid listed; the last column indicates whether the SNP is known or not; the presence of known SNPs in variant protein HUMCACH1A_PEA_{—}1_P2 (SEQ ID NO:792) sequence provides support for the deduced sequence of this variant protein according to the present invention).

TABLE 11
Nucleic acid SNPs

| SNP position on nucleotide | Alternative | |
|---|---|---|
| sequence | nucleic acid | Previously known SNP? |
| 331 | T -> A | Yes |
| 422 | C -> T | Yes |
| 458 | T -> C | Yes |
| 970 | T -> C | Yes |
| 1615 | C -> T | Yes |
| 2242 | T -> G | Yes |
| 2296 | C -> T | No |
| 2381 | T -> C | Yes |
| 2937 | A -> | No |
| 3271 | C -> T | Yes |
| 3624 | T -> C | No |
| 3637 | C -> T | Yes |
| 6919 | C -> T | Yes |
| 7383 | A -> G | Yes |
| 7701 | A -> G | Yes |
| 8070 | C -> T | Yes |

The coding portion of transcript HUMCACH1A_PEA_{—}1_T4 (SEQ ID NO:709) is shown in bold; this coding portion starts at position 512 and ends at position 7054. The transcript also has the following SNPs as listed in Table 12 (given according to their position on the nucleotide sequence, with the alternative nucleic acid listed; the last column indicates whether the SNP is known or not; the presence of known SNPs in variant protein HUMCACH1A_PEA_{—}1_P2 (SEQ ID NO:792) sequence provides support for the deduced sequence of this variant protein according to the present invention).

TABLE 12
Nucleic acid SNPs

| SNP position on nucleotide | Alternative | |
|---|---|---|
| sequence | nucleic acid | Previously known SNP? |
| 331 | T -> A | Yes |
| 422 | C -> T | Yes |
| 458 | T -> C | Yes |
| 970 | T -> C | Yes |
| 1615 | C -> T | Yes |
| 2242 | T -> G | Yes |
| 2296 | C -> T | No |
| 2381 | T -> C | Yes |
| 2937 | A -> | No |
| 3271 | C -> T | Yes |
| 3624 | T -> C | No |
| 3637 | C -> T | Yes |
| 6919 | C -> T | Yes |
| 7383 | A -> G | Yes |
| 7701 | A -> G | Yes |

Variant protein HUMCACH1A_PEA_{—}1_P3 (SEQ ID NO:793) according to the present invention has an amino acid sequence as given at the end of the application; it is encoded by transcript(s) HUMCACH1A_PEA_{—}1_T6 (SEQ ID NO:710). The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein HUMCACH1A_PEA_{—}1_P3 (SEQ ID NO:793) also has the following non-silent SNPs (Single Nucleotide Polymorphisms) as listed in Table 13, (given according to their position(s) on the amino acid sequence, with the alternative amino acid(s) listed; the last column indicates whether the SNP is known or not; the presence of known SNPs in variant protein HUMCACH1A_PEA_{—}1_P3 (SEQ ID NO:793) sequence provides support for the deduced sequence of this variant protein according to the present invention).

TABLE 13
Amino acid mutations

| SNP position(s) on amino acid | Alternative | |
|---|---|---|
| sequence | amino acid(s) | Previously known SNP? |
| 577 | I -> M | Yes |
| 809 | N -> | No |
| 1038 | I -> T | No |

Variant protein HUMCACH1A_PEA_{—}1_P3 (SEQ ID NO:793) is encoded by the following transcript(s): HUMCACH1A_PEA_{—}1_T6 (SEQ ID NO:710), for which the sequence(s) is/are given at the end of the application. The coding portion of transcript HUMCACH1A_PEA_{—}1_T6 (SEQ ID NO:710) is shown in bold; this coding portion starts at position 512 and ends at position 6157. The transcript also has the following SNPs as listed in Table 14 (given according to their position on the nucleotide sequence, with the alternative nucleic acid listed; the last column indicates whether the SNP is known or not; the presence of known SNPs in variant protein HUMCACH1A_PEA_{—}1_P3 (SEQ ID NO:793) sequence provides support for the deduced sequence of this variant protein according to the present invention).

TABLE 14
Nucleic acid SNPs

| SNP position on nucleotide | Alternative | |
|---|---|---|
| sequence | nucleic acid | Previously known SNP? |
| 331 | T -> A | Yes |
| 422 | C -> T | Yes |
| 458 | T -> C | Yes |
| 970 | T -> C | Yes |
| 1615 | C -> T | Yes |
| 2242 | T -> G | Yes |
| 2296 | C -> T | No |
| 2381 | T -> C | Yes |
| 2937 | A -> | No |
| 3271 | C -> T | Yes |
| 3624 | T -> C | No |
| 3637 | C -> T | Yes |
| 6312 | G -> A | Yes |
| 7098 | C -> T | Yes |
| 7562 | A -> G | Yes |
| 7880 | A -> G | Yes |
| 8249 | C -> T | Yes |
| 8296 | T -> A | Yes |
| 8540 | T -> C | Yes |
| 8723 | G -> A | Yes |
| 8811 | A -> C | Yes |
| 8864 | A -> G | Yes |
| 9207 | G -> A | Yes |
| 9554 | G -> | No |
| 9554 | G -> A | No |

Variant protein HUMCACH1A_PEA_{—}1_P4 (SEQ ID NO:794) according to the present invention has an amino acid sequence as given at the end of the application; it is encoded by transcript(s) HUMCACH1A_PEA_{—}1_T7 (SEQ ID NO:711). The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein HUMCACH1A_PEA_{—}1_P4 (SEQ ID NO:794) also has the following non-silent SNPs (Single Nucleotide Polymorphisms) as listed in Table 15, (given according to their position(s) on the amino acid sequence, with the alternative amino acid(s) listed; the last column indicates whether the SNP is known or not; the presence of known SNPs in variant protein HUMCACH1A_PEA_{—}1_P4 (SEQ ID NO:794) sequence provides support for the deduced sequence of this variant protein according to the present invention).

TABLE 15
Amino acid mutations

| SNP position(s) on amino acid | Alternative | |
|---|---|---|
| sequence | amino acid(s) | Previously known SNP? |
| 577 | I -> M | Yes |
| 809 | N -> | No |
| 1038 | I -> T | No |

Variant protein HUMCACH1A_PEA_{—}1_P4 (SEQ ID NO:794) is encoded by the following transcript(s): HUMCACH1A_PEA_{—}1_T7 (SEQ ID NO:711), for which the sequence(s) is/are given at the end of the application. The coding portion of transcript HUMCACH1A_PEA_{—}1_T7 (SEQ ID NO:711) is shown in bold; this coding portion starts at position 512 and ends at position 7027. The transcript also has the following SNPs as listed in Table 16 (given according to their position on the nucleotide sequence, with the alternative nucleic acid listed; the last column indicates whether the SNP is known or not; the presence of known SNPs in variant protein HUMCACH1A_PEA_{—}1_P4 (SEQ ID NO:794) sequence provides support for the deduced sequence of this variant protein according to the present invention).

TABLE 16
Nucleic acid SNPs

| SNP position on nucleotide | Alternative | |
|---|---|---|
| sequence | nucleic acid | Previously known SNP? |
| 331 | T -> A | Yes |
| 422 | C -> T | Yes |
| 458 | T -> C | Yes |
| 970 | T -> C | Yes |
| 1615 | C -> T | Yes |
| 2242 | T -> G | Yes |
| 2296 | C -> T | No |
| 2381 | T -> C | Yes |
| 2937 | A -> | No |
| 3271 | C -> T | Yes |
| 3624 | T -> C | No |
| 3637 | C -> T | Yes |
| 6892 | C -> T | Yes |
| 7356 | A -> G | Yes |
| 7674 | A -> G | Yes |
| 8043 | C -> T | Yes |
| 8090 | T -> A | Yes |
| 8334 | T -> C | Yes |
| 8517 | G -> A | Yes |
| 8605 | A -> C | Yes |
| 8658 | A -> G | Yes |
| 9001 | G -> A | Yes |
| 9348 | G -> | No |
| 9348 | G -> A | No |

Variant protein HUMCACH1A_PEA_{—}1_P5 (SEQ ID NO:795) according to the present invention has an amino acid sequence as given at the end of the application; it is encoded by transcript(s) HUMCACH1A_PEA_{—}1_T8 (SEQ ID NO:712). The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein HUMCACH1A_PEA_{—}1_P5 (SEQ ID NO:795) also has the following non-silent SNPs (Single Nucleotide Polymorphisms) as listed in Table 17, (given according to their position(s) on the amino acid sequence, with the alternative amino acid(s) listed; the last column indicates whether the SNP is known or not; the presence of known SNPs in variant protein HUMCACH1A_PEA_{—}1_P5 (SEQ ID NO:795) sequence provides support for the deduced sequence of this variant protein according to the present invention).

TABLE 17
Amino acid mutations

| SNP position(s) on amino acid | Alternative | |
|---|---|---|
| sequence | amino acid(s) | Previously known SNP? |
| 557 | I -> M | Yes |
| 789 | N -> | No |
| 1018 | I -> T | No |

Variant protein HUMCACH1A_PEA_{—}1_P5 (SEQ ID NO:795) is encoded by the following transcript(s): HUMCACH1A_PEA_{—}1_T8 (SEQ ID NO:712), for which the sequence(s) is/are given at the end of the application. The coding portion of transcript HUMCACH1A_PEA_{—}1_T8 (SEQ ID NO:712) is shown in bold; this coding portion starts at position 512 and ends at position 6994. The transcript also has the following SNPs as listed in Table 18 (given according to their position on the nucleotide sequence, with the alternative nucleic acid listed; the last column indicates whether the SNP is known or not; the presence of known SNPs in variant protein HUMCACH1A_PEA_{—}1_P5 (SEQ ID NO:795) sequence provides support for the deduced sequence of this variant protein according to the present invention).

TABLE 18
Nucleic acid SNPs

| SNP position on nucleotide | Alternative | |
|---|---|---|
| sequence | nucleic acid | Previously known SNP? |
| 331 | T -> A | Yes |
| 422 | C -> T | Yes |
| 458 | T -> C | Yes |
| 970 | T -> C | Yes |
| 1615 | C -> T | Yes |
| 2182 | T -> G | Yes |
| 2236 | C -> T | No |
| 2321 | T -> C | Yes |
| 2877 | A -> | No |
| 3211 | C -> T | Yes |
| 3564 | T -> C | No |
| 3577 | C -> T | Yes |
| 6859 | C -> T | Yes |
| 7323 | A -> G | Yes |
| 7641 | A -> G | Yes |
| 8010 | C -> T | Yes |
| 8057 | T -> A | Yes |
| 8301 | T -> C | Yes |
| 8484 | G -> A | Yes |
| 8572 | A -> C | Yes |
| 8625 | A -> G | Yes |
| 8968 | G -> A | Yes |
| 9315 | G -> | No |
| 9315 | G -> A | No |

Variant protein HUMCACH1A_PEA_{—}1_P7 (SEQ ID NO:796) according to the present invention has an amino acid sequence as given at the end of the application; it is encoded by transcript(s) HUMCACH1A_PEA_{—}1_T12 (SEQ ID NO:713). An alignment is given to the known protein (Voltage-dependent L-type calcium channel alpha-1D subunit (SEQ ID NO:790)) at the end of the application. One or more alignments to one or more previously published protein sequences are given at the end of the application. A brief description of the relationship of the variant protein according to the present invention to each such aligned protein is as follows:

Comparison Report Between HUMCACH1A_PEA_{—}1_P7 (SEQ ID NO:796) and CCAD_HUMAN_V3 (SEQ ID NO:791):

1. An isolated chimeric polypeptide encoding for HUMCACH1A_PEA_{—}1_P7 (SEQ ID NO:796), comprising a first amino acid sequence being at least 90% homologous to MPTSETESVNTENVSGEGENRGCCGSL corresponding to amino acids 466-492 of CCAD_HUMAN_V3 (SEQ ID NO:791), which also corresponds to amino acids 1-27 of HUMCACH1A_PEA_{—}1_P7 (SEQ ID NO:796), a second amino acid sequence being at least 70%, optionally at least 80%, preferably at least 85%, more preferably at least 90% and most preferably at least 95% homologous to a polypeptide having the sequence WCWWRRRGAAKAGPSGCRRWG (SEQ ID NO:1573) corresponding to amino acids 28-48 of HUMCACH1A_PEA_{—}1_P7 (SEQ ID NO:796), and a third amino acid sequence being at least 90% homologous to QAISKSKLSRRWRRWNRFNRRRCRAAVKSVTFYWLVIVLVFLNTLTISSEHYNQPDWLTQIQDIANKVLL ALFTCEMLVKMYSLGLQAYFVSLFNRFDCFVVCGGITETILVELEIMSPLGISVFRCVRLLRIFKVTRHWTS LSNLVASLLNSMKSIASLLLLLFLFIIIFSLLGMQLFGGKFNFDETQTKRSTFDNFPQALLTVFQILTGEDWN AVMYDGIMAYGGPSSSGMIVCIYFIILFICGNYILLNVFLAIAVDNLADAESLNTAQKEEAEEKERKKIARK ESLENKKNNKPEVNQIANSDNKVTIDDYREEDEDKDPYPPCDVPVGEEEEEEEEDEPEVPAGPRPRRISELN MKEKIAPIPEGSAFFILSKTNPIRVGCHKLINHHIFTNLILVFIMLSSAALAAEDPIRSHSFRNTILGYFDYAFT AIFTVEILLKMTTFGAFLHKGAFCRNYFNLLDMLVVGVSLVSFGIQSSAISVVKILRVLRVLRPLRAINRAK GLKHVVQCVFVAIRTIGNIMIVTTLLQFMFACIGVQLFKGKFYRCTDEAKSNPEECRGLFILYKDGDVDSP VVRERIWQNSDFNFDNVLSAMMALFTVSTFEGWPALLYKAIDSNGENIGPIYNHRVEISIFFIIYIIIVAFFM MNIFVGFVIVTFQEQGEKEYKNCELDKNQRQCVEYALKARPLRRYIPKNPYQYKFWYVVNSSPFEYMMF VLIMLNTLCLAMQHYEQSKMFNDAMDILNMVFTGVFTVEMVLKVIAFKPKGYFSDAWNTFDSLIVIGSIID VALSEADPTESENVPVPTATPGNSEESNRISITFFRLFRVMRLVKLLSRGEGIRTLLWTFIKSFQALPYVALLI AMLFFIYAVIGMQMFGKVAMRDNNQINRNNNFQTFPQAVLLLFRCATGEAWQEIMLACLPGKLCDPESD YNPGEEYTCGSNFAIVYFISFYMLCAFLIINLFVAVIMDNFDYLTRDWSILGPHHLDEFKRIWSEYDPEAKG RIKHLDVVTLLRRIQPPLGFGKLCPHRVACKRLVAMNMPLNSDGTVMFNATLFALVRTALKIKTEGNLEQ ANEELRAVIKKIWKKTSMKLLDQVVPPAGDDEVTVGKFYATFLIQDYFRKFKKRKEQGLVGKYPAKNTTI ALQAGLRTLHDIGPEIRRAISCDLQDDEPEETKREEEDDVFKRNGALLGNHVNHVNSDRRDSLQQTNTTH RPLHVQRPSIPPASDTEKPLFPPAGNSVCHNHHNHNSIGKQVPTSTNANLNNANMSKAAHGKRPSIGNLEH VSENGHHSSHKHDREPQRRSSVKRTRYYETYIRSDSGDEQLPTICREDPEIHGYFRDPHCLGEQEYFSSEEC YEDDSSPTWSRQNYGYYSRYPGRNIDSERPRGYHHPQGFLEDDDSPVCYDSRRSPRRRLLPPTPASHRRSS FNFECLRRQSSQEEVPSSPIFPHRTALPLHLMQQQIMAVAGLDSSKAQKYSPSHSTRSWATPPATPPYRDW TPCYTPLIQVEQSEALDQVNGSLPSLHRSSWYTDEPDISYRTFTPASLTVPSSFRNKNSDKQRSADSLVEAV LISEGLGRYARDPKFVSATKHEIADACDLTIDEMESAASTLLNGNVRPRANGDVGPLSHRQDYELQDFGPG YSDEEPDPGRDEEDLADEMICITTL corresponding to amino acids 494-2161 of CCAD_HUMAN_V3 (SEQ ID NO:791), which also corresponds to amino acids 49-1716 of HUMCACH1A_PEA_{—}1_P7 (SEQ ID NO:796), wherein said first amino acid sequence, second amino acid sequence and third amino acid sequence are contiguous and in a sequential order.

2. An isolated polypeptide encoding for an edge portion of HUMCACH1A_PEA_{—}1_P7 (SEQ ID NO:796), comprising an amino acid sequence being at least 70%, optionally at least about 80%, preferably at least about 85%, more preferably at least about 90% and most preferably at least about 95% homologous to the sequence encoding for WCWWRRRGAAKAGPSGCRRWG (SEQ ID NO:1573), corresponding to HUMCACH1A_PEA_{—}1_P7 (SEQ ID NO:796).

3. A bridge portion of HUMCACH1A_PEA_{—}1_P7 (SEQ ID NO:796), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise L, having a structure as follows (numbering according to HUMCACH1A_PEA_{—}1_P7 (SEQ ID NO:796)): a sequence starting from any of amino acid numbers 492−x to 492; and ending at any of amino acid numbers 28+((n−2)−x), in which x varies from 0 to n−2.

It should be noted that the known protein sequence (CCAD_HUMAN (SEQ ID NO:790)) has one or more changes than the sequence given at the end of the application and named as being the amino acid sequence for CCAD_HUMAN_V3 (SEQ ID NO:791). These changes were previously known to occur and are listed in the table below.

TABLE 19
Changes to CCAD_HUMAN_V3 (SEQ ID NO: 791)

| SNP position(s) on amino | |
|---|---|
| acid sequence | Type of change |
| 638 | conflict |
| 651 | conflict |
| 1347 | conflict |
| 1434 | conflict |

The location or the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein HUMCACH1A_PEA_{—}1_P7 (SEQ ID NO:796) also has the following non-silent SNPs (Single Nucleotide Polymorphisms) as listed in Table 20, (given according to their position(s) on the amino acid sequence, with the alternative amino acid(s) listed; the last column indicates whether the SNP is known or not; the presence of known SNPs in variant protein HUMCACH1A_PEA_{—}1_P7 (SEQ ID NO:796) sequence provides support for the deduced sequence of this variant protein according to the present invention).

TABLE 20
Amino acid mutations

| SNP position(s) on amino acid | Alternative | |
|---|---|---|
| sequence | amino acid(s) | Previously known SNP? |
| 112 | I -> M | Yes |
| 344 | N -> | No |
| 573 | I -> T | No |

Variant protein HUMCACH1A_PEA_{—}1_P7 (SEQ ID NO:796) is encoded by the following transcript(s): HUMCACH1A_PEA_{—}1_T12 (SEQ ID NO:713), for which the sequence(s) is/are given at the end of the application. The coding portion of transcript HUMCACH1A_PEA_{—}1_T12 (SEQ ID NO:713) is shown in bold; this coding portion starts at position 240 and ends at position 5387. The transcript also has the following SNPs as listed in Table 21 (given according to their position on the nucleotide sequence, with the alternative nucleic acid listed; the last column indicates whether the SNP is known or not; the presence of known SNPs in variant protein HUMCACH1A_PEA_{—}1_P7 (SEQ ID NO:796) sequence provides support for the deduced sequence of this variant protein according to the present invention).

TABLE 21
Nucleic acid SNPs

| SNP position on nucleotide | Alternative | |
|---|---|---|
| sequence | nucleic acid | Previously known SNP? |
| 575 | T -> G | Yes |
| 629 | C -> T | No |
| 714 | T -> C | Yes |
| 1270 | A -> | No |
| 1604 | C -> T | Yes |
| 1957 | T -> C | No |
| 1970 | C -> T | Yes |
| 5252 | C -> T | Yes |
| 5716 | A -> G | Yes |
| 6034 | A -> G | Yes |
| 6403 | C -> T | Yes |
| 6450 | T -> A | Yes |
| 6694 | T -> C | Yes |
| 6877 | G -> A | Yes |
| 6965 | A -> C | Yes |
| 7018 | A -> G | Yes |
| 7361 | G -> A | Yes |
| 7708 | G -> | No |
| 7708 | G -> A | No |

Variant protein HUMCACH1A_PEA_{—}1_P8 (SEQ ID NO:797) according to the present invention has an amino acid sequence as given at the end of the application; it is encoded by transcript(s) HUMCACH1A_PEA_{—}1_T13 (SEQ ID NO:714). The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein HUMCACH1A_PEA_{—}1_P8 (SEQ ID NO:797) also has the following non-silent SNPs (Single Nucleotide Polymorphisms) as listed in Table 22, (given according to their position(s) on the amino acid sequence, with the alternative amino acid(s) listed; the last column indicates whether the SNP is known or not; the presence of known SNPs in variant protein HUMCACH1A_PEA_{—}1_P8 (SEQ ID NO:797) sequence provides support for the deduced sequence of this variant protein according to the present invention).

TABLE 22
Amino acid mutations

| SNP position(s) on amino acid | Alternative | |
|---|---|---|
| sequence | amino acid(s) | Previously known SNP? |
| 577 | I -> M | Yes |
| 809 | N -> | No |
| 1038 | I -> T | No |

Variant protein HUMCACH1A_PEA_{—}1_P8 (SEQ ID NO:797) is encoded by the following transcript(s): HUMCACH1A_PEA_{—}1_T13 (SEQ ID NO:714), for which the sequence(s) is/are given at the end of the application. The coding portion of transcript HUMCACH1A_PEA_{—}1_T13 (SEQ ID NO:714) is shown in bold; this coding portion starts at position 512 and ends at position 88889. The transcript also has the following SNPs as listed in Table 23 (given according to their position on the nucleotide sequence, with the alternative nucleic acid listed; the last column indicates whether the SNP is known or not; the presence of known SNPs in variant protein HUMCACH1A_PEA_{—}1_P8 (SEQ ID NO:797) sequence provides support for the deduced sequence of this variant protein according to the present invention).

TABLE 23
Nucleic acid SNPs

| SNP position on nucleotide | Alternative | |
|---|---|---|
| sequence | nucleic acid | Previously known SNP? |
| 331 | T -> A | Yes |
| 422 | C -> T | Yes |
| 458 | T -> C | Yes |
| 970 | T -> C | Yes |
| 1615 | C -> T | Yes |
| 2242 | T -> G | Yes |
| 2296 | C -> T | No |
| 2381 | T -> C | Yes |
| 2937 | A -> | No |
| 3271 | C -> T | Yes |
| 3624 | T -> C | No |
| 3637 | C -> T | Yes |

Variant protein HUMCACH1A_PEA_{—}1_P9 (SEQ ID NO:798) according to the present invention has an amino acid sequence as given at the end of the application; it is encoded by transcript(s) HUMCACH1A_PEA_{—}1_T14 (SEQ ID NO:715). The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein HUMCACH1A_PEA_{—}1_P9 (SEQ ID NO:798) also has the following non-silent SNPs (Single Nucleotide Polymorphisms) as listed in Table 24, (given according to their position(s) on the amino acid sequence, with the alternative amino acid(s) listed; the last column indicates whether the SNP is known or not; the presence of known SNPs in variant protein HUMCACH1A_PEA_{—}1_P9 (SEQ ID NO:798) sequence provides support for the deduced sequence of this variant protein according to the present invention).

TABLE 24
Amino acid mutations

| SNP position(s) on amino acid | Alternative | |
|---|---|---|
| sequence | amino acid(s) | Previously known SNP? |
| 577 | I -> M | Yes |
| 809 | N -> | No |
| 1038 | I -> T | No |

Variant protein HUMCACH1A_PEA_{—}1_P9 (SEQ ID NO:798) is encoded by the following transcript(s): HUMCACH1A_PEA_{—}1_T14 (SEQ ID NO:715), for which the sequence(s) is/are given at the end of the application. The coding portion of transcript HUMCACH1A_PEA_{—}1_T14 (SEQ ID NO:715) is shown in bold; this coding portion starts at position 512 and ends at position 5386. The transcript also has the following SNPs as listed in Table 25 (given according to their position on the nucleotide sequence, with the alternative nucleic acid listed; the last column indicates whether the SNP is known or not; the presence of known SNPs in variant protein HUMCACH1A_PEA_{—}1_P9 (SEQ ID NO:798) sequence provides support for the deduced sequence of this variant protein according to the present invention).

TABLE 25
Nucleic acid SNPs

| SNP position on nucleotide | Alternative | |
|---|---|---|
| sequence | nucleic acid | Previously known SNP? |
| 331 | T -> A | Yes |
| 422 | C -> T | Yes |
| 458 | T -> C | Yes |
| 970 | T -> C | Yes |
| 1615 | C -> T | Yes |
| 2242 | T -> G | Yes |
| 2296 | C -> T | No |
| 2381 | T -> C | Yes |
| 2937 | A -> | No |
| 3271 | C -> T | Yes |
| 3624 | T -> C | No |
| 3637 | C -> T | Yes |

Variant protein HUMCACH1A_PEA_{—}1_P10 (SEQ ID NO:799) according to the present invention has an amino acid sequence as given at the end of the application; it is encoded by transcript(s) HUMCACH1A_PEA_{—}1_T15 (SEQ ID NO:716). The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein HUMCACH1A_PEA_{—}1_P10 (SEQ ID NO:799) also has the following non-silent SNPs (Single Nucleotide Polymorphisms) as listed in Table 26, (given according to their position(s) on the amino acid sequence, with the alternative amino acid(s) listed; the last column indicates whether the SNP is known or not; the presence of known SNPs in variant protein HUMCACH1A_PEA_{—}1_P10 (SEQ ID NO:799) sequence provides support for the deduced sequence of this variant protein according to the present invention).

TABLE 26
Amino acid mutations

| SNP position(s) | | |
|---|---|---|
| on amino acid | Alternative | Previously |
| sequence | amino acid(s) | known SNP? |
| 577 | I -> M | Yes |
| 809 | N -> | No |
| 1038 | I -> T | No |

Variant protein HUMCACH1A_PEA_{—}1_P10 (SEQ ID NO:799) is encoded by the following transcript(s): HUMCACH1A_PEA_{—}1_T15 (SEQ ID NO:716), for which the sequence(s) is/are given at the end of the application. The coding portion of transcript HUMCACH1A_PEA_{—}1_T15 (SEQ ID NO:716) is shown in bold; this coding portion starts at position 512 and ends at position 88889. The transcript also has the following SNPs as listed in Table 27 (given according to their position on the nucleotide sequence, with the alternative nucleic acid listed; the last column indicates whether the SNP is known or not; the presence of known SNPs in variant protein HUMCACH1A_PEA_{—}1_P10 (SEQ ID NO:799) sequence provides support for the deduced sequence of this variant protein according to the present invention).

TABLE 27
Nucleic acid SNPs

| SNP position | | |
|---|---|---|
| on nucleotide | Alternative | Previously |
| sequence | nucleic acid | known SNP? |
| 331 | T -> A | Yes |
| 422 | C -> T | Yes |
| 458 | T -> C | Yes |
| 970 | T -> C | Yes |
| 1615 | C -> T | Yes |
| 2242 | T -> G | Yes |
| 2296 | C -> T | No |
| 2381 | T -> C | Yes |
| 2937 | A -> | No |
| 3271 | C -> T | Yes |
| 3624 | T -> C | No |
| 3637 | C -> T | Yes |

Variant protein HUMCACH1A_PEA_{—}1_P11 (SEQ ID NO:800) according to the present invention has an amino acid sequence as given at the end of the application; it is encoded by transcript(s) HUMCACH1A_PEA_{—}1_T16 (SEQ ID NO:717). The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein HUMCACH1A_PEA_{—}1_P11 (SEQ ID NO:800) also has the following non-silent SNPs (Single Nucleotide Polymorphisms) as listed in Table 28, (given according to their position(s) on the amino acid sequence, with the alternative amino acid(s) listed; the last column indicates whether the SNP is known or not; the presence of known SNPs in variant protein HUMCACH1A_PEA_{—}1_P11 (SEQ ID NO:800) sequence provides support for the deduced sequence of this variant protein according to the present invention).

TABLE 28
Amino acid mutations

| SNP position(s) | | |
|---|---|---|
| on amino acid | Alternative | Previously |
| sequence | amino acid(s) | known SNP? |
| 577 | I -> M | Yes |
| 809 | N -> | No |

Variant protein HUMCACH1A_PEA_{—}1_P11 (SEQ ID NO:800) is encoded by the following transcript(s): HUMCACH1A_PEA_{—}1_T16 (SEQ ID NO:717), for which the sequence(s) is/are given at the end of the application. The coding portion of transcript HUMCACH1A_PEA_{—}1_T16 (SEQ ID NO:717) is shown in bold; this coding portion starts at position 512 and ends at position 88889. The transcript also has the following SNPs as listed in Table 29 (given according to their position on the nucleotide sequence, with the alternative nucleic acid listed; the last column indicates whether the SNP is known or not; the presence of known SNPs in variant protein HUMCACH1A_PEA_{—}1_P11 (SEQ ID NO:800) sequence provides support for the deduced sequence of this variant protein according to the present invention).

TABLE 29
Nucleic acid SNPs

| SNP position | | |
|---|---|---|
| on nucleotide | Alternative | Previously |
| sequence | nucleic acid | known SNP? |
| 331 | T -> A | Yes |
| 422 | C -> T | Yes |
| 458 | T -> C | Yes |
| 970 | T -> C | Yes |
| 1615 | C -> T | Yes |
| 2242 | T -> G | Yes |
| 2296 | C -> T | No |
| 2381 | T -> C | Yes |
| 2937 | A -> | No |
| 3271 | C -> T | Yes |

Variant protein HUMCACH1A_PEA_{—}1_P12 (SEQ ID NO:801) according to the present invention has an amino acid sequence as given at the end of the application; it is encoded by transcript(s) HUMCACH1A_PEA_{—}1_T17 (SEQ ID NO:718). The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein HUMCACH1A_PEA_{—}1_P12 (SEQ ID NO:801) is encoded by the following transcript(s): HUMCACH1A_PEA_{—}1_T17 (SEQ ID NO:718), for which the sequence(s) is/are given at the end of the application. The coding portion of transcript HUMCACH1A_PEA_{—}1_T17 (SEQ ID NO:718) is shown in bold; this coding portion starts at position 1 and ends at position 2644. The transcript also has the following SNPs as listed in Table 30 (given according to their position on the nucleotide sequence, with the alternative nucleic acid listed; the last column indicates whether the SNP is known or not; the presence of known SNPs in variant protein HUMCACH1A_PEA_{—}1_P12 (SEQ ID NO:801) sequence provides support for the deduced sequence of this variant protein according to the present invention).

TABLE 30
Nucleic acid SNPs

| SNP position | | |
|---|---|---|
| on nucleotide | Alternative | Previously |
| sequence | nucleic acid | known SNP? |
| 2509 | C -> T | Yes |
| 2973 | A -> G | Yes |
| 3291 | A -> G | Yes |
| 3660 | C -> T | Yes |
| 3707 | T -> A | Yes |
| 3951 | T -> C | Yes |
| 4134 | G -> A | Yes |
| 4222 | A -> C | Yes |
| 4275 | A -> G | Yes |
| 4618 | G -> A | Yes |
| 4965 | G -> | No |
| 4965 | G -> A | No |

Variant protein HUMCACH1A_PEA_{—}1_P13 (SEQ ID NO:802) according to the present invention has an amino acid sequence as given at the end of the application; it is encoded by transcript(s) HUMCACH1A_PEA_{—}1_T18 (SEQ ID NO:719). An alignment is given to the known protein (Voltage-dependent L-type calcium channel alpha-1D subunit (SEQ ID NO:790)) at the end of the application. One or more alignments to one or more previously published protein sequences are given at the end of the application. A brief description of the relationship of the variant protein according to the present invention to each such aligned protein is as follows:

Comparison Report Between HUMCACH1A_PEA_{—}1_P13 (SEQ ID NO:802) and CCAD_HUMAN (SEQ ID NO:790):

1. An isolated chimeric polypeptide encoding for HUMCACH1A_PEA_{—}1_P13 (SEQ ID NO:802), comprising a first amino acid sequence being at least 70%, optionally at least 80%, preferably at least 85%, more preferably at least 90% and most preferably at least 95% homologous to a polypeptide having the sequence MLRPRCLLRRTAHPPHSAPAPAPARSKCLGSWSNVLIRESSVWSLRL (SEQ ID NO:1477) corresponding to amino acids 1-47 of HUMCACH1A_PEA_{—}1_P13 (SEQ ID NO:802), and a second amino acid sequence being at least 90% homologous to DDEVTVGKFYATFLIQDYFRKFKKRKEQGLVGKYPAKNTTIALQAGLRTLHDIGPEIRRAISCDLQDDEPE ETKREEEDDVFKRNGALLGNHVNHVNSDRRDSLQQTNTTHRPLHVQRPSIPPASDTEKPLFPPAGNSVCH NHHNHNSIGKQVPTSTNANLNNANMSKAAHGKRPSIGNLEHVSENGHHSSHKHDREPQRRSSVKRTRYY ETYIRSDSGDEQLPTICREDPEIHGYFRDPHCLGEQEYFSSEECYEDDSSPTWSRQNYGYYSRYPGRNIDSE RPRGYHHPQGFLEDDDSPVCYDSRRSPRRRLLPPTPASHRRSSFNFECLRRQSSQEEVPSSPIFPHRTALPLH LMQQQIMAVAGLDSSKAQKYSPSHSTRSWATPPATPPYRDWTPCYTPLIQVEQSEALDQVNGSLPSLHRSS WYTDEPDISYRTFTPASLTVPSSFRNKNSDKQRSADSLVEAVLISEGLGRYARDPKFVSATKHEIADACDLT IDEMESAASTLLNGNVRPRANGDVGPLSHRQDYELQDFGPGYSDEEPDPGRDEEDLADEMICITTL corresponding to amino acids 1598-2161 of CCAD_HUMAN (SEQ ID NO:790), which also corresponds to amino acids 48-611 of HUMCACH1A_PEA_{—}1_P13 (SEQ ID NO:802), wherein said first amino acid sequence and second amino acid sequence are contiguous and in a sequential order.

2. An isolated polypeptide encoding for a head of HUMCACH1A_PEA_{—}1_P13 (SEQ ID NO:802), comprising a polypeptide being at least 70%, optionally at least about 80%, preferably at least about 85%, more preferably at least about 90% and most preferably at least about 95% homologous to the sequence MLRPRCLLRRTAHPPHSAPAPAPARSKCLGSWSNVLIRESSVWSLRL (SEQ ID NO:1477) of HUMCACH1A_PEA_{—}1_P13 (SEQ ID NO:802).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

The glycosylation sites of variant protein HUMCACH1A_PEA_{—}1_P13 (SEQ ID NO:802), as compared to the known protein Voltage-dependent L-type calcium channel alpha-1D subunit (SEQ ID NO:790), are described in Table 31 (given according to their position(s) on the amino acid sequence in the first column; the second column indicates whether the glycosylation site is present in the variant protein; and the last column indicates whether the position is different on the variant protein).

TABLE 31
Glycosylation site(s)

| Position(s) on | | |
|---|---|---|
| known amino acid | Present in | Position in |
| sequence | variant protein? | variant protein? |
| 225 | no | |
| 155 | no | |
| 329 | no | |

The phosphorilation sites of variant protein HUMCACH1A_PEA_{—}1_P13 (SEQ ID NO:802), as compared to the known protein Voltage-dependent L-type calcium channel alpha-1D subunit (SEQ ID NO:790), are described in Table 32 (given according to their position(s) on the amino acid sequence in the first column; the second column indicates whether the phosphorilation site is present in the variant protein; and the last column indicates whether the position is different on the variant protein).

TABLE 32
Phosphorilation site(s)

| Position(s) on | | |
|---|---|---|
| known amino acid | Present in | Position in |
| sequence | variant protein? | variant protein? |
| 1475 | no | |

Variant protein HUMCACH1A_PEA_{—}1_P13 (SEQ ID NO:802) is encoded by the following transcript(s): HUMCACH1A_PEA_{—}1_T18 (SEQ ID NO:719), for which the sequence(s) is/are given at the end of the application. The coding portion of transcript HUMCACH1A_PEA_{—}1_T18 (SEQ ID NO:719) is shown in bold; this coding portion starts at position 63 and ends at position 1895. The transcript also has the following SNPs as listed in Table 33 (given according to their position on the nucleotide sequence, with the alternative nucleic acid listed; the last column indicates whether the SNP is known or not; the presence of known SNPs in variant protein HUMCACH1A_PEA_{—}1_P13 (SEQ ID NO:802) sequence provides support for the deduced sequence of this variant protein according to the present invention).

TABLE 33
Nucleic acid SNPs

| SNP position on | | |
|---|---|---|
| nucleotide | Alternative | Previously |
| sequence | nucleic acid | known SNP? |
| 1760 | C -> T | Yes |
| 2224 | A -> G | Yes |
| 2542 | A -> G | Yes |
| 2911 | C -> T | Yes |
| 2958 | T -> A | Yes |
| 3202 | T -> C | Yes |
| 3385 | G -> A | Yes |
| 3473 | A -> C | Yes |
| 3526 | A -> G | Yes |
| 3869 | G -> A | Yes |
| 4216 | G -> | No |
| 4216 | G -> A | No |

Variant protein HUMCACH1A_PEA_{—}1_P14 (SEQ ID NO:803) according to the present invention has an amino acid sequence as given at the end of the application; it is encoded by transcript(s) HUMCACH1A_PEA_{—}1_T19 (SEQ ID NO:720). An alignment is given to the known protein (Voltage-dependent L-type calcium channel alpha-1D subunit (SEQ ID NO:790)) at the end of the application. One or more alignments to one or more previously published protein sequences are given at the end of the application. A brief description of the relationship of the variant protein according to the present invention to each such aligned protein is as follows:

Comparison Report Between HUMCACH1A_PEA_{—}1_P14 (SEQ ID NO:803) and CCAD_HUMAN (SEQ ID NO:790):

1. An isolated chimeric polypeptide encoding for HUMCACH1A_PEA_{—}1_P14 (SEQ ID NO:803), comprising a first amino acid sequence being at least 90% homologous to MSKAAHGKRPSIGNLEHVSENGHHSSHKHDREPQRRSSVKRTRYYETYIRSDSGDEQLPTICREDPEIHGYF RDPHCLGEQEYFSSEECYEDDSSPTWSRQNYGYYSRYPGRNIDSERPRGYHHPQGFLEDDDSPVCYDSRRS PRRRLLPPTPASHRRSSFNFECLRRQSSQEEVPSSPIFPHRTALPLHLMQQQIMAVAGLDSSKAQKYSPSHST RSWATPPATPPYRDWTPCYTPLIQVEQSEALDQVNGSLPSLHRSSWYTDEPDISYRTFTPASLTVPSSFRNK NSDKQRSADSLVEAVLISEGLGRYARDPKFVSATKHEIADACDLTIDEMESAASTLLNGNVRPRANGDVG PLSHRQDYELQDFGPGYSDEEPDPGRDEEDLADEMICITTL corresponding to amino acids 1763-2161 of CCAD_HUMAN (SEQ ID NO:790), which also corresponds to amino acids 1-399 of HUMCACH1A_PEA_{—}1_P14 (SEQ ID NO:803).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

The glycosylation sites of variant protein HUMCACH1A_PEA_{—}1_P14 (SEQ ID NO:803), as compared to the known protein Voltage-dependent L-type calcium channel alpha-1D subunit (SEQ ID NO:790), are described in Table 34 (given according to their position(s) on the amino acid sequence in the first column; the second column indicates whether the glycosylation site is present in the variant protein; and the last column indicates whether the position is different on the variant protein).

TABLE 34
Glycosylation site(s)

| Position(s) on | | |
|---|---|---|
| known amino acid | Present in | Position in |
| sequence | variant protein? | variant protein? |
| 225 | no | |
| 155 | no | |
| 329 | no | |

The phosphorilation sites of variant protein HUMCACH1A_PEA_{—}1_P14 (SEQ ID NO:803), as compared to the known protein Voltage-dependent L-type calcium channel alpha-1D subunit (SEQ ID NO:790), are described in Table 35 (given according to their position(s) on the amino acid sequence in the first column; the second column indicates whether the phosphorilation site is present in the variant protein; and the last column indicates whether the position is different on the variant protein).

TABLE 35
Phosphorilation site(s)

| Position(s) on | | |
|---|---|---|
| known amino acid | Present in | Position in |
| sequence | variant protein? | variant protein? |
| 1475 | no | |

Variant protein HUMCACH1A_PEA_{—}1_P14 (SEQ ID NO:803) is encoded by the following transcript(s): HUMCACH1A_PEA_{—}1_T19 (SEQ ID NO:720), for which the sequence(s) is/are given at the end of the application. The coding portion of transcript HUMCACH1A_PEA_{—}1_T19 (SEQ ID NO:720) is shown in bold; this coding portion starts at position 1820 and ends at position 3016. The transcript also has the following SNPs as listed in Table 36 (given according to their position on the nucleotide sequence, with the alternative nucleic acid listed; the last column indicates whether the SNP is known or not; the presence of known SNPs in variant protein HUMCACH1A_PEA_{—}1_P14 (SEQ ID NO:803) sequence provides support for the deduced sequence of this variant protein according to the present invention).

TABLE 36
Nucleic acid SNPs

| SNP position | | |
|---|---|---|
| on nucleotide | Alternative | Previously |
| sequence | nucleic acid | known SNP? |
| 84 | G -> T | Yes |
| 542 | G -> A | Yes |
| 579 | G -> A | Yes |
| 690 | A -> G | Yes |
| 890 | C -> T | Yes |
| 2881 | C -> T | Yes |
| 3345 | A -> G | Yes |
| 3663 | A -> G | Yes |
| 4032 | C -> T | Yes |
| 4079 | T -> A | Yes |
| 4323 | T -> C | Yes |
| 4506 | G -> A | Yes |
| 4594 | A -> C | Yes |
| 4647 | A -> G | Yes |
| 4990 | G -> A | Yes |
| 5337 | G -> | No |
| 5337 | G -> A | No |

Variant protein HUMCACH1A_PEA_{—}1_P15 (SEQ ID NO:804) according to the present invention has an amino acid sequence as given at the end of the application; it is encoded by transcript(s) HUMCACH1A_PEA_{—}1_T20 (SEQ ID NO:721). The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein HUMCACH1A_PEA_{—}1_P15 (SEQ ID NO:804) is encoded by the following transcript(s): HUMCACH1A_PEA_{—}1_T20 (SEQ ID NO:721), for which the sequence(s) is/are given at the end of the application. The coding portion of transcript HUMCACH1A_PEA_{—}1_T20 (SEQ ID NO:721) is shown in bold; this coding portion starts at position 512 and ends at position 1732. The transcript also has the following SNPs as listed in Table 37 (given according to their position on the nucleotide sequence, with the alternative nucleic acid listed; the last column indicates whether the SNP is known or not; the presence of known SNPs in variant protein HUMCACH1A_PEA_{—}1_P15 (SEQ ID NO:804) sequence provides support for the deduced sequence of this variant protein according to the present invention).

TABLE 37
Nucleic acid SNPs

| SNP position | | |
|---|---|---|
| on nucleotide | Alternative | Previously |
| sequence | nucleic acid | known SNP? |
| 331 | T -> A | Yes |
| 422 | C -> T | Yes |
| 458 | T -> C | Yes |
| 970 | T -> C | Yes |
| 1615 | C -> T | Yes |

Variant protein HUMCACH1A_PEA_{—}1_P17 (SEQ ID NO:805) according to the present invention has an amino acid sequence as given at the end of the application; it is encoded by transcript(s) HUMCACH1A_PEA_{—}1_T22 (SEQ ID NO:722). An alignment is given to the known protein (Voltage-dependent L-type calcium channel alpha-1D subunit (SEQ ID NO:790)) at the end of the application. One or more alignments to one or more previously published protein sequences are given at the end of the application. A brief description of the relationship of the variant protein according to the present invention to each such aligned protein is as follows:

Comparison Report Between HUMCACH1A_PEA_{—}1_P17 (SEQ ID NO:805) and CCAD_HUMAN (SEQ ID NO:790):

1. An isolated chimeric polypeptide encoding for HUMCACH1A_PEA_{—}1_P17 (SEQ ID NO:805), comprising a first amino acid sequence being at least 90% homologous to MMMMMMMKKMQHQRQQQADHANEANYARGTRLPLSGEGPTSQPNSSKQTVLSWQAAIDAARQAKAA QTMSTSAPPPVGSLSQRKRQQYAKSKKQGNSSNSRPARALFCLSLNNPIRRACISIVEWKPFDIFILLAIFAN CVALAIYIPFPEDDSNSTNHNLEKVEYAFLIIFTVETFLKIIAYGLLLHPNAYVRNGWNLLDFVIVIVGLFSVI LEQLTKETEGGNHSSGKSGGFDVKALRAFRVLRPLRLVSGVPSLQVVLNSIIKAMVPLLHIALLVLFVIIIYA IIGLELFIGKMHKTCFFADSDIVAEEDPAPCAFSGNGRQCTANGTECRSGWVGPNGGITNFDNFAFAMLTV FQCITMEGWTDVLYWMNDAMGFELPWVYFVSLVIFGSFFVLNLVLGVLSG corresponding to amino acids 1-407 of CCAD_HUMAN (SEQ ID NO:790), which also corresponds to amino acids 1-407 of HUMCACH1A_PEA_{—}1_P17 (SEQ ID NO:805), and a second amino acid sequence being at least 70%, optionally at least 80%, preferably at least 85%, more preferably at least 90% and most preferably at least 95% homologous to a polypeptide having the sequence HGGSRL (SEQ ID NO:1478) corresponding to amino acids 408-413 of HUMCACH1A_PEA_{—}1_P17 (SEQ ID NO:805), wherein said first amino acid sequence and second amino acid sequence are contiguous and in a sequential order.

2. An isolated polypeptide encoding for a tail of HUMCACH1A_PEA_{—}1_P17 (SEQ ID NO:805), comprising a polypeptide being at least 70%, optionally at least about 80%, preferably at least about 85%, more preferably at least about 90% and most preferably at least about 95% homologous to the sequence HGGSRL (SEQ ID NO:1478) in HUMCACH1A_PEA_{—}1_P17 (SEQ ID NO:805).

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein HUMCACH1A_PEA_{—}1_P17 (SEQ ID NO:805) also has the following non-silent SNPs (Single Nucleotide Polymorphisms) as listed in Table 38, (given according to their position(s) on the amino acid sequence, with the alternative amino acid(s) listed; the last column indicates whether the SNP is known or not; the presence of known SNPs in variant protein HUMCACH1A_PEA_{—}1_P17 (SEQ ID NO:805) sequence provides support for the deduced sequence of this variant protein according to the present invention).

TABLE 38
Amino acid mutations

| SNP position(s) | | |
|---|---|---|
| on amino acid | Alternative | Previously |
| sequence | amino acid(s) | known SNP? |
| 409 | G -> S | No |

The glycosylation sites or variant protein HUMCACH1A_PEA_{—}1_P17 (SEQ ID NO:805), as compared to the known protein Voltage-dependent L-type calcium channel alpha-1D subunit (SEQ ID NO:790), are described in Table 39 (given according to their position(s) on the amino acid sequence in the first column; the second column indicates whether the glycosylation site is present in the variant protein; and the last column indicates whether the position is different on the variant protein).

TABLE 39
Glycosylation site(s)

| Position(s) on | | |
|---|---|---|
| known amino acid | Present in | Position in |
| sequence | variant protein? | variant protein? |
| 225 | yes | 225 |
| 155 | yes | 155 |
| 329 | yes | 329 |

The phosphorilation sites of variant protein HUMCACH1A_PEA_{—}1_P17 (SEQ ID NO:805), as compared to the known protein Voltage-dependent L-type calcium channel alpha-1D subunit (SEQ ID NO:790), are described in Table 40 (given according to their position(s) on the amino acid sequence in the first column; the second column indicates whether the phosphorilation site is present in the variant protein; and the last column indicates whether the position is different on the variant protein).

TABLE 40
Phosphorilation site(s)

| Position(s) on | | |
|---|---|---|
| known amino acid | Present in | Position in |
| sequence | variant protein? | variant protein? |
| 1475 | no | |

Variant protein HUMCACH1A_PEA_{—}1_P17 (SEQ ID NO:805) is encoded by the following transcript(s): HUMCACH1A_PEA_{—}1_T22 (SEQ ID NO:722), for which the sequence(s) is/are given at the end of the application. The coding portion of transcript HUMCACH1A_PEA_{—}1_T22 (SEQ ID NO:722) is shown in bold; this coding portion starts at position 512 and ends at position 1750. The transcript also has the following SNPs as listed in Table 41 (given according to their position on the nucleotide sequence, with the alternative nucleic acid listed; the last column indicates whether the SNP is known or not; the presence of known SNPs in variant protein HUMCACH1A_PEA_{—}1_P17 (SEQ ID NO:805) sequence provides support for the deduced sequence of this variant protein according to the present invention).

TABLE 41
Nucleic acid SNPs

| SNP position on nucleotide | Alternative | |
|---|---|---|
| sequence | nucleic acid | Previously known SNP? |
| 331 | T -> A | Yes |
| 422 | C -> T | Yes |
| 458 | T -> C | Yes |
| 970 | T -> C | Yes |
| 1615 | C -> T | Yes |
| 1736 | G -> A | No |
| 1848 | G -> A | No |
| 2004 | A -> T | Yes |
| 2006 | C -> T | Yes |
| 2020 | A -> | Yes |
| 2045 | A -> G | Yes |
| 2235 | G -> A | Yes |
| 2432 | C -> A | Yes |
| 2565 | T -> A | Yes |
| 2749 | C -> T | Yes |
| 2887 | C -> T | Yes |
| 3099 | A -> G | Yes |
| 3319 | C -> T | Yes |

As noted above, cluster HUMCACH1A features 67 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HUMCACH1A_PEA_{—}1_node_{—}2 (SEQ ID NO:723) according to the present invention is supported by 1 libraries. The number of libraries was determined as previously described. This segment can be found in the following transcript(s): HUMCACH1A_PEA_{—}1_T0 (SEQ ID NO:705), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716), HUMCACH1A_PEA_{—}1_T16 (SEQ ID NO:717), HUMCACH1A_PEA_{—}1_T20 (SEQ ID NO:721) and HUMCACH1A_PEA_{—}1_T22 (SEQ ID NO:722). Table 42 below describes the starting and ending position of this segment on each transcript.

TABLE 42
Segment location on transcripts

| | Segment | Segment |
|---|---|---|
| | starting | ending |
| Transcript name | position | position |
| HUMCACH1A_PEA_1_T0 (SEQ ID NO: 705) | 1 | 468 |
| HUMCACH1A_PEA_1_T2 (SEQ ID NO: 707) | 1 | 468 |
| HUMCACH1A_PEA_1_T3 (SEQ ID NO: 708) | 1 | 468 |
| HUMCACH1A_PEA_1_T4 (SEQ ID NO: 709) | 1 | 468 |
| HUMCACH1A_PEA_1_T6 (SEQ ID NO: 710) | 1 | 468 |
| HUMCACH1A_PEA_1_T7 (SEQ ID NO: 711) | 1 | 468 |
| HUMCACH1A_PEA_1_T8 (SEQ ID NO: 712) | 1 | 468 |
| HUMCACH1A_PEA_1_T13 (SEQ ID NO: | 1 | 468 |
| 714) | | |
| HUMCACH1A_PEA_1_T14 (SEQ ID NO: | 1 | 468 |
| 715) | | |
| HUMCACH1A_PEA_1_T15 (SEQ ID NO: | 1 | 468 |
| 716) | | |
| HUMCACH1A_PEA_1_T16 (SEQ ID NO: | 1 | 468 |
| 717) | | |
| HUMCACH1A_PEA_1_T20 (SEQ ID NO: | 1 | 468 |
| 721) | | |
| HUMCACH1A_PEA_1_T22 (SEQ ID NO: | 1 | 468 |
| 722) | | |

Segment cluster HUMCACH1A_PEA_{—}1_node_{—}5 (SEQ ID NO:724) according to the present invention is supported by 3 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716), HUMCACH1A_PEA_{—}1_T16 (SEQ ID NO:717), HUMCACH1A_PEA_{—}1_T20 (SEQ ID NO:721) and HUMCACH1A_PEA_{—}1_T22 (SEQ ID NO:722). Table 43 below describes the starting and ending position of this segment on each transcript.

TABLE 43
Segment location on transcripts

| | Segment | Segment |
|---|---|---|
| | starting | ending |
| Transcript name | position | position |
| HUMCACH1A_PEA_1_T0 (SEQ ID NO: 705) | 579 | 888 |
| HUMCACH1A_PEA_1_T1 (SEQ ID NO: 706) | 156 | 465 |
| HUMCACH1A_PEA_1_T2 (SEQ ID NO: 707) | 579 | 888 |
| HUMCACH1A_PEA_1_T3 (SEQ ID NO: 708) | 579 | 888 |
| HUMCACH1A_PEA_1_T4 (SEQ ID NO: 709) | 579 | 888 |
| HUMCACH1A_PEA_1_T6 (SEQ ID NO: 710) | 579 | 888 |
| HUMCACH1A_PEA_1_T7 (SEQ ID NO: 711) | 579 | 888 |
| HUMCACH1A_PEA_1_T8 (SEQ ID NO: 712) | 579 | 888 |
| HUMCACH1A_PEA_1_T13 (SEQ ID NO: | 579 | 888 |
| 714) | | |
| HUMCACH1A_PEA_1_T14 (SEQ ID NO: | 579 | 888 |
| 715) | | |
| HUMCACH1A_PEA_1_T15 (SEQ ID NO: | 579 | 888 |
| 716) | | |
| HUMCACH1A_PEA_1_T16 (SEQ ID NO: | 579 | 888 |
| 717) | | |
| HUMCACH1A_PEA_1_T20 (SEQ ID NO: | 579 | 888 |
| 721) | | |
| HUMCACH1A_PEA_1_T22 (SEQ ID NO: | 579 | 888 |
| 722) | | |

Segment cluster HUMCACH1A_PEA_{—}1_node_{—}9 (SEQ ID NO:725) according to the present invention is supported by 0 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716), HUMCACH1A_PEA_{—}1_T16 (SEQ ID NO:717), HUMCACH1A_PEA_{—}1_T20 (SEQ ID NO:721) and HUMCACH1A_PEA_{—}1_T22 (SEQ ID NO:722). Table 44 below describes the starting and ending position of this segment on each transcript.

TABLE 44
Segment location on transcripts

| | Segment | Segment |
|---|---|---|
| | starting | ending |
| Transcript name | position | position |
| HUMCACH1A_PEA_1_T0 (SEQ ID NO: 705) | 995 | 1134 |
| HUMCACH1A_PEA_1_T1 (SEQ ID NO: 706) | 572 | 711 |
| HUMCACH1A_PEA_1_T2 (SEQ ID NO: 707) | 995 | 1134 |
| HUMCACH1A_PEA_1_T3 (SEQ ID NO: 708) | 995 | 1134 |
| HUMCACH1A_PEA_1_T4 (SEQ ID NO: 709) | 995 | 1134 |
| HUMCACH1A_PEA_1_T6 (SEQ ID NO: 710) | 995 | 1134 |
| HUMCACH1A_PEA_1_T7 (SEQ ID NO: 711) | 995 | 1134 |
| HUMCACH1A_PEA_1_T8 (SEQ ID NO: 712) | 995 | 1134 |
| HUMCACH1A_PEA_1_T13 (SEQ ID NO: | 995 | 1134 |
| 714) | | |
| HUMCACH1A_PEA_1_T14 (SEQ ID NO: | 995 | 1134 |
| 715) | | |
| HUMCACH1A_PEA_1_T15 (SEQ ID NO: | 995 | 1134 |
| 716) | | |
| HUMCACH1A_PEA_1_T16 (SEQ ID NO: | 995 | 1134 |
| 717) | | |
| HUMCACH1A_PEA_1_T20 (SEQ ID NO: | 995 | 1134 |
| 721) | | |
| HUMCACH1A_PEA_1_T22 (SEQ ID NO: | 995 | 1134 |
| 722) | | |

Segment cluster HUMCACH1A_PEA_{—}1_node_{—}11 (SEQ ID NO:726) according to the present invention is supported by 4 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716), HUMCACH1A_PEA_{—}1_T16 (SEQ ID NO:717), HUMCACH1A_PEA_{—}1_T20 (SEQ ID NO:721) and HUMCACH1A_PEA_{—}1_T22 (SEQ ID NO:722). Table 45 below describes the starting and ending position of this segment on each transcript.

TABLE 45
Segment location on transcripts

| | Segment | Segment |
|---|---|---|
| | starting | ending |
| Transcript name | position | position |
| HUMCACH1A_PEA_1_T0 (SEQ ID NO: 705) | 1135 | 1277 |
| HUMCACH1A_PEA_1_T1 (SEQ ID NO: 706) | 712 | 854 |
| HUMCACH1A_PEA_1_T2 (SEQ ID NO: 707) | 1135 | 1277 |
| HUMCACH1A_PEA_1_T3 (SEQ ID NO: 708) | 1135 | 1277 |
| HUMCACH1A_PEA_1_T4 (SEQ ID NO: 709) | 1135 | 1277 |
| HUMCACH1A_PEA_1_T6 (SEQ ID NO: 710) | 1135 | 1277 |
| HUMCACH1A_PEA_1_T7 (SEQ ID NO: 711) | 1135 | 1277 |
| HUMCACH1A_PEA_1_T8 (SEQ ID NO: 712) | 1135 | 1277 |
| HUMCACH1A_PEA_1_T13 (SEQ ID NO: | 1135 | 1277 |
| 714) | | |
| HUMCACH1A_PEA_1_T14 (SEQ ID NO: | 1135 | 1277 |
| 715) | | |
| HUMCACH1A_PEA_1_T15 (SEQ ID NO: | 1135 | 1277 |
| 716) | | |
| HUMCACH1A_PEA_1_T16 (SEQ ID NO: | 1135 | 1277 |
| 717) | | |
| HUMCACH1A_PEA_1_T20 (SEQ ID NO: | 1135 | 1277 |
| 721) | | |
| HUMCACH1A_PEA_1_T22 (SEQ ID NO: | 1135 | 1277 |
| 722) | | |

Segment cluster HUMCACH1A_PEA_{—}1_node_{—}14 (SEQ ID NO:727) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716), HUMCACH1A_PEA_{—}1_T16 (SEQ ID NO:717), HUMCACH1A_PEA_{—}1_T20 (SEQ ID NO:721) and HUMCACH1A_PEA_{—}1_T22 (SEQ ID NO:722). Table 46 below describes the starting and ending position of this segment on each transcript.

TABLE 46
Segment location on transcripts

| | Segment | Segment |
|---|---|---|
| | starting | ending |
| Transcript name | position | position |
| HUMCACH1A_PEA_1_T0 (SEQ ID NO: 705) | 1278 | 1430 |
| HUMCACH1A_PEA_1_T1 (SEQ ID NO: 706) | 855 | 1007 |
| HUMCACH1A_PEA_1_T2 (SEQ ID NO: 707) | 1278 | 1430 |
| HUMCACH1A_PEA_1_T3 (SEQ ID NO: 708) | 1278 | 1430 |
| HUMCACH1A_PEA_1_T4 (SEQ ID NO: 709) | 1278 | 1430 |
| HUMCACH1A_PEA_1_T6 (SEQ ID NO: 710) | 1278 | 1430 |
| HUMCACH1A_PEA_1_T7 (SEQ ID NO: 711) | 1278 | 1430 |
| HUMCACH1A_PEA_1_T8 (SEQ ID NO: 712) | 1278 | 1430 |
| HUMCACH1A_PEA_1_T13 (SEQ ID NO: | 1278 | 1430 |
| 714) | | |
| HUMCACH1A_PEA_1_T14 (SEQ ID NO: | 1278 | 1430 |
| 715) | | |
| HUMCACH1A_PEA_1_T15 (SEQ ID NO: | 1278 | 1430 |
| 716) | | |
| HUMCACH1A_PEA_1_T16 (SEQ ID NO: | 1278 | 1430 |
| 717) | | |
| HUMCACH1A_PEA_1_T20 (SEQ ID NO: | 1278 | 1430 |
| 721) | | |
| HUMCACH1A_PEA_1_T22 (SEQ ID NO: | 1278 | 1430 |
| 722) | | |

Segment cluster HUMCACH1A_PEA_{—}1_node_{—}16 (SEQ ID NO:728) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716), HUMCACH1A_PEA_{—}1_T16 (SEQ ID NO:717), HUMCACH1A_PEA_{—}1_T20 (SEQ ID NO:721) and HUMCACH1A_PEA_{—}1_T22 (SEQ ID NO:722). Table 47 below describes the starting and ending position of this segment on each transcript.

TABLE 47
Segment location on transcripts

| | Segment | Segment |
|---|---|---|
| | starting | ending |
| Transcript name | position | position |
| HUMCACH1A_PEA_1_T0 (SEQ ID NO: 705) | 1431 | 1627 |
| HUMCACH1A_PEA_1_T1 (SEQ ID NO: 706) | 1008 | 1204 |
| HUMCACH1A_PEA_1_T2 (SEQ ID NO: 707) | 1431 | 1627 |
| HUMCACH1A_PEA_1_T3 (SEQ ID NO: 708) | 1431 | 1627 |
| HUMCACH1A_PEA_1_T4 (SEQ ID NO: 709) | 1431 | 1627 |
| HUMCACH1A_PEA_1_T6 (SEQ ID NO: 710) | 1431 | 1627 |
| HUMCACH1A_PEA_1_T7 (SEQ ID NO: 711) | 1431 | 1627 |
| HUMCACH1A_PEA_1_T8 (SEQ ID NO: 712) | 1431 | 1627 |
| HUMCACH1A_PEA_1_T13 (SEQ ID NO: | 1431 | 1627 |
| 714) | | |
| HUMCACH1A_PEA_1_T14 (SEQ ID NO: | 1431 | 1627 |
| 715) | | |
| HUMCACH1A_PEA_1_T15 (SEQ ID NO: | 1431 | 1627 |
| 716) | | |
| HUMCACH1A_PEA_1_T16 (SEQ ID NO: | 1431 | 1627 |
| 717) | | |
| HUMCACH1A_PEA_1_T20 (SEQ ID NO: | 1431 | 1627 |
| 721) | | |
| HUMCACH1A_PEA_1_T22 (SEQ ID NO: | 1431 | 1627 |
| 722) | | |

Segment cluster HUMCACH1A_PEA_{—}1_node_{—}27 (SEQ ID NO:729) according to the present invention is supported by 1 libraries. The number of libraries was determined as previously described. This segment can be found in the following transcript(s): HUMCACH1A_PEA_{—}1_T20 (SEQ ID NO:721). Table 48 below describes the starting and ending position of this segment on each transcript.

TABLE 48
Segment location on transcripts

| | Segment | Segment |
|---|---|---|
| Transcript name | starting position | ending position |
| HUMCACH1A_PEA_1_T20 (SEQ | 1732 | 1973 |
| ID NO: 721) | | |

Segment cluster HUMCACH1A_PEA_{—}1_node_{—}30 (SEQ ID NO:730) according to the present invention is supported by 8 libraries. The number of libraries was determined as previously described. This segment can be found in the following transcript(s): HUMCACH1A_PEA_{—}1_T22 (SEQ ID NO:722). Table 49 below describes the starting and ending position of this segment on each transcript.

TABLE 49
Segment location on transcripts

| | Segment | Segment |
|---|---|---|
| Transcript name | starting position | ending position |
| HUMCACH1A_PEA_1_T22 (SEQ | 1732 | 3379 |
| ID NO: 722) | | |

Segment cluster HUMCACH1A_PEA_{—}1_node_{—}33 (SEQ ID NO:731) according to the present invention is supported by 3 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716) and HUMCACH1A_PEA_{—}1_T16 (SEQ ID NO:717). Table 50 below describes the starting and ending position of this segment on each transcript.

TABLE 50
Segment location on transcripts

| | Segment | Segment |
|---|---|---|
| | starting | ending |
| Transcript name | position | position |
| HUMCACH1A_PEA_1_T0 (SEQ ID NO: 705) | 1732 | 1901 |
| HUMCACH1A_PEA_1_T1 (SEQ ID NO: 706) | 1309 | 1478 |
| HUMCACH1A_PEA_1_T2 (SEQ ID NO: 707) | 1732 | 1901 |
| HUMCACH1A_PEA_1_T3 (SEQ ID NO: 708) | 1732 | 1901 |
| HUMCACH1A_PEA_1_T4 (SEQ ID NO: 709) | 1732 | 1901 |
| HUMCACH1A_PEA_1_T6 (SEQ ID NO: 710) | 1732 | 1901 |
| HUMCACH1A_PEA_1_T7 (SEQ ID NO: 711) | 1732 | 1901 |
| HUMCACH1A_PEA_1_T8 (SEQ ID NO: 712) | 1732 | 1901 |
| HUMCACH1A_PEA_1_T12 (SEQ ID NO: | 65 | 234 |
| 713) | | |
| HUMCACH1A_PEA_1_T13 (SEQ ID NO: | 1732 | 1901 |
| 714) | | |
| HUMCACH1A_PEA_1_T14 (SEQ ID NO: | 1732 | 1901 |
| 715) | | |
| HUMCACH1A_PEA_1_T15 (SEQ ID NO: | 1732 | 1901 |
| 716) | | |
| HUMCACH1A_PEA_1_T16 (SEQ ID NO: | 1732 | 1901 |
| 717) | | |

Segment cluster HUMCACH1A_PEA_{—}1_node_{—}41 (SEQ ID NO:732) according to the present invention is supported by 5 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716) and HUMCACH1A_PEA_{—}1_T16 (SEQ ID NO:717). Table 51 below describes the starting and ending position of this segment on each transcript.

TABLE 51
Segment location on transcripts

| | Segment | Segment |
|---|---|---|
| | starting | ending |
| Transcript name | position | position |
| HUMCACH1A_PEA_1_T0 (SEQ ID NO: 705) | 2077 | 2237 |
| HUMCACH1A_PEA_1_T1 (SEQ ID NO: 706) | 1654 | 1814 |
| HUMCACH1A_PEA_1_T2 (SEQ ID NO: 707) | 2077 | 2237 |
| HUMCACH1A_PEA_1_T3 (SEQ ID NO: 708) | 2077 | 2237 |
| HUMCACH1A_PEA_1_T4 (SEQ ID NO: 709) | 2077 | 2237 |
| HUMCACH1A_PEA_1_T6 (SEQ ID NO: 710) | 2077 | 2237 |
| HUMCACH1A_PEA_1_T7 (SEQ ID NO: 711) | 2077 | 2237 |
| HUMCACH1A_PEA_1_T8 (SEQ ID NO: 712) | 2017 | 2177 |
| HUMCACH1A_PEA_1_T12 (SEQ ID NO: | 410 | 570 |
| 713) | | |
| HUMCACH1A_PEA_1_T13 (SEQ ID NO: | 2077 | 2237 |
| 714) | | |
| HUMCACH1A_PEA_1_T14 (SEQ ID NO: | 2077 | 2237 |
| 715) | | |
| HUMCACH1A_PEA_1_T15 (SEQ ID NO: | 2077 | 2237 |
| 716) | | |
| HUMCACH1A_PEA_1_T16 (SEQ ID NO: | 2077 | 2237 |
| 717) | | |

Segment cluster HUMCACH1A_PEA_{—}1_node_{—}43 (SEQ ID NO:733) according to the present invention is supported by 5 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716) and HUMCACH1A_PEA_{—}1_T16 (SEQ ID NO:717). Table 52 below describes the starting and ending position of this segment on each transcript.

TABLE 52
Segment location on transcripts

| | Segment | Segment |
|---|---|---|
| | starting | ending |
| Transcript name | position | position |
| HUMCACH1A_PEA_1_T0 (SEQ ID NO: 705) | 2238 | 2463 |
| HUMCACH1A_PEA_1_T1 (SEQ ID NO: 706) | 1815 | 2040 |
| HUMCACH1A_PEA_1_T2 (SEQ ID NO: 707) | 2238 | 2463 |
| HUMCACH1A_PEA_1_T3 (SEQ ID NO: 708) | 2238 | 2463 |
| HUMCACH1A_PEA_1_T4 (SEQ ID NO: 709) | 2238 | 2463 |
| HUMCACH1A_PEA_1_T6 (SEQ ID NO: 710) | 2238 | 2463 |
| HUMCACH1A_PEA_1_T7 (SEQ ID NO: 711) | 2238 | 2463 |
| HUMCACH1A_PEA_1_T8 (SEQ ID NO: 712) | 2178 | 2403 |
| HUMCACH1A_PEA_1_T12 (SEQ ID NO: | 571 | 796 |
| 713) | | |
| HUMCACH1A_PEA_1_T13 (SEQ ID NO: | 2238 | 2463 |
| 714) | | |
| HUMCACH1A_PEA_1_T14 (SEQ ID NO: | 2238 | 2463 |
| 715) | | |
| HUMCACH1A_PEA_1_T15 (SEQ ID NO: | 2238 | 2463 |
| 716) | | |
| HUMCACH1A_PEA_1_T16 (SEQ ID NO: | 2238 | 2463 |
| 717) | | |

Segment cluster HUMCACH1A_PEA_{—}1_node_{—}45 (SEQ ID NO:734) according to the present invention is supported by 4 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716) and HUMCACH1A_PEA_{—}1_T16 (SEQ ID NO:717). Table 53 below describes the starting and ending position of this segment on each transcript.

TABLE 53
Segment location on transcripts

| | Segment | Segment |
|---|---|---|
| | starting | ending |
| Transcript name | position | position |
| HUMCACH1A_PEA_1_T0 (SEQ ID NO: 705) | 2464 | 2671 |
| HUMCACH1A_PEA_1_T1 (SEQ ID NO: 706) | 2041 | 2248 |
| HUMCACH1A_PEA_1_T2 (SEQ ID NO: 707) | 2464 | 2671 |
| HUMCACH1A_PEA_1_T3 (SEQ ID NO: 708) | 2464 | 2671 |
| HUMCACH1A_PEA_1_T4 (SEQ ID NO: 709) | 2464 | 2671 |
| HUMCACH1A_PEA_1_T6 (SEQ ID NO: 710) | 2464 | 2671 |
| HUMCACH1A_PEA_1_T7 (SEQ ID NO: 711) | 2464 | 2671 |
| HUMCACH1A_PEA_1_T8 (SEQ ID NO: 712) | 2404 | 2611 |
| HUMCACH1A_PEA_1_T12 (SEQ ID NO: | 797 | 1004 |
| 713) | | |
| HUMCACH1A_PEA_1_T13 (SEQ ID NO: | 2464 | 2671 |
| 714) | | |
| HUMCACH1A_PEA_1_T14 (SEQ ID NO: | 2464 | 2671 |
| 715) | | |
| HUMCACH1A_PEA_1_T15 (SEQ ID NO: | 2464 | 2671 |
| 716) | | |
| HUMCACH1A_PEA_1_T16 (SEQ ID NO: | 2464 | 2671 |
| 717) | | |

Segment cluster HUMCACH1A_PEA_{—}1_node_{—}47 (SEQ ID NO:735) according to the present invention is supported by 3 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716) and HUMCACH1A_PEA_{—}1_T16 (SEQ ID NO:717). Table 54 below describes the starting and ending position of this segment on each transcript.

TABLE 54
Segment location on transcripts

| | Segment | Segment |
|---|---|---|
| | starting | ending |
| Transcript name | position | position |
| HUMCACH1A_PEA_1_T0 (SEQ ID NO: 705) | 2672 | 2792 |
| HUMCACH1A_PEA_1_T1 (SEQ ID NO: 706) | 2249 | 2369 |
| HUMCACH1A_PEA_1_T2 (SEQ ID NO: 707) | 2672 | 2792 |
| HUMCACH1A_PEA_1_T3 (SEQ ID NO: 708) | 2672 | 2792 |
| HUMCACH1A_PEA_1_T4 (SEQ ID NO: 709) | 2672 | 2792 |
| HUMCACH1A_PEA_1_T6 (SEQ ID NO: 710) | 2672 | 2792 |
| HUMCACH1A_PEA_1_T7 (SEQ ID NO: 711) | 2672 | 2792 |
| HUMCACH1A_PEA_1_T8 (SEQ ID NO: 712) | 2612 | 2732 |
| HUMCACH1A_PEA_1_T12 (SEQ ID NO: | 1005 | 1125 |
| 713) | | |
| HUMCACH1A_PEA_1_T13 (SEQ ID NO: | 2672 | 2792 |
| 714) | | |
| HUMCACH1A_PEA_1_T14 (SEQ ID NO: | 2672 | 2792 |
| 715) | | |
| HUMCACH1A_PEA_1_T15 (SEQ ID NO: | 2672 | 2792 |
| 716) | | |
| HUMCACH1A_PEA_1_T16 (SEQ ID NO: | 2672 | 2792 |
| 717) | | |

Segment cluster HUMCACH1A_PEA_{—}1_node_{—}55 (SEQ ID NO:736) according to the present invention is supported by 6 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716) and HUMCACH1A_PEA_{—}1_T16 (SEQ ID NO:717). Table 55 below describes the starting and ending position of this segment on each transcript.

TABLE 55
Segment location on transcripts

| | Segment | Segment |
|---|---|---|
| | starting | ending |
| Transcript name | position | position |
| HUMCACH1A_PEA_1_T0 (SEQ ID NO: 705) | 3045 | 3192 |
| HUMCACH1A_PEA_1_T1 (SEQ ID NO: 706) | 2622 | 2769 |
| HUMCACH1A_PEA_1_T2 (SEQ ID NO: 707) | 3045 | 3192 |
| HUMCACH1A_PEA_1_T3 (SEQ ID NO: 708) | 3045 | 3192 |
| HUMCACH1A_PEA_1_T4 (SEQ ID NO: 709) | 3045 | 3192 |
| HUMCACH1A_PEA_1_T6 (SEQ ID NO: 710) | 3045 | 3192 |
| HUMCACH1A_PEA_1_T7 (SEQ ID NO: 711) | 3045 | 3192 |
| HUMCACH1A_PEA_1_T8 (SEQ ID NO: 712) | 2985 | 3132 |
| HUMCACH1A_PEA_1_T12 (SEQ ID NO: | 1378 | 1525 |
| 713) | | |
| HUMCACH1A_PEA_1_T13 (SEQ ID NO: | 3045 | 3192 |
| 714) | | |
| HUMCACH1A_PEA_1_T14 (SEQ ID NO: | 3045 | 3192 |
| 715) | | |
| HUMCACH1A_PEA_1_T15 (SEQ ID NO: | 3045 | 3192 |
| 716) | | |
| HUMCACH1A_PEA_1_T16 (SEQ ID NO: | 3045 | 3192 |
| 717) | | |

Segment cluster HUMCACH1A_PEA_{—}1_node_{—}57 (SEQ ID NO:737) according to the present invention is supported by 7 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716) and HUMCACH1A_PEA_{—}1_T16 (SEQ ID NO:717). Table 56 below describes the starting and ending position of this segment on each transcript.

TABLE 56
Segment location on transcripts

| | Seg- | |
|---|---|---|
| | ment | Segment |
| | starting | ending |
| Transcript name | position | position |
| HUMCACH1A_PEA_1_T0 (SEQ ID NO: 705) | 3193 | 3322 |
| HUMCACH1A_PEA_1_T1 (SEQ ID NO: 706) | 2770 | 2899 |
| HUMCACH1A_PEA_1_T2 (SEQ ID NO: 707) | 3193 | 3322 |
| HUMCACH1A_PEA_1_T3 (SEQ ID NO: 708) | 3193 | 3322 |
| HUMCACH1A_PEA_1_T4 (SEQ ID NO: 709) | 3193 | 3322 |
| HUMCACH1A_PEA_1_T6 (SEQ ID NO: 710) | 3193 | 3322 |
| HUMCACH1A_PEA_1_T7 (SEQ ID NO: 711) | 3193 | 3322 |
| HUMCACH1A_PEA_1_T8 (SEQ ID NO: 712) | 3133 | 3262 |
| HUMCACH1A_PEA_1_T12 (SEQ ID NO: 713) | 1526 | 1655 |
| HUMCACH1A_PEA_1_T13 (SEQ ID NO: 714) | 3193 | 3322 |
| HUMCACH1A_PEA_1_T14 (SEQ ID NO: 715) | 3193 | 3322 |
| HUMCACH1A_PEA_1_T15 (SEQ ID NO: 716) | 3193 | 3322 |
| HUMCACH1A_PEA_1_T16 (SEQ ID NO: 717) | 3193 | 3322 |

Segment cluster HUMCACH1A_PEA_{—}1_node_{—}70 (SEQ ID NO:738) according to the present invention is supported by 3 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T15 (SEQ ID NO:716). Table 57 below describes the starting and ending position of this segment on each transcript.

TABLE 57
Segment location on transcripts

| | Seg- | |
|---|---|---|
| | ment | Segment |
| | starting | ending |
| Transcript name | position | position |
| HUMCACH1A_PEA_1_T0 (SEQ ID NO: 705) | 3739 | 3885 |
| HUMCACH1A_PEA_1_T1 (SEQ ID NO: 706) | 3316 | 3462 |
| HUMCACH1A_PEA_1_T2 (SEQ ID NO: 707) | 3739 | 3885 |
| HUMCACH1A_PEA_1_T3 (SEQ ID NO: 708) | 3739 | 3885 |
| HUMCACH1A_PEA_1_T4 (SEQ ID NO: 709) | 3739 | 3885 |
| HUMCACH1A_PEA_1_T6 (SEQ ID NO: 710) | 3739 | 3885 |
| HUMCACH1A_PEA_1_T7 (SEQ ID NO: 711) | 3739 | 3885 |
| HUMCACH1A_PEA_1_T8 (SEQ ID NO: 712) | 3679 | 3825 |
| HUMCACH1A_PEA_1_T12 (SEQ ID NO: 713) | 2072 | 2218 |
| HUMCACH1A_PEA_1_T13 (SEQ ID NO: 714) | 3739 | 3885 |
| HUMCACH1A_PEA_1_T14 (SEQ ID NO: 715) | 3739 | 3885 |
| HUMCACH1A_PEA_1_T15 (SEQ ID NO: 716) | 3739 | 3885 |

Segment cluster HUMCACH1A_PEA_{—}1_node_{—}72 (SEQ ID NO:739) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T15 (SEQ ID NO:716). Table 58 below describes the starting and ending position of this segment on each transcript.

TABLE 58
Segment location on transcripts

| | Seg- | |
|---|---|---|
| | ment | Segment |
| | starting | ending |
| Transcript name | position | position |
| HUMCACH1A_PEA_1_T0 (SEQ ID NO: 705) | 3886 | 4087 |
| HUMCACH1A_PEA_1_T1 (SEQ ID NO: 706) | 3463 | 3664 |
| HUMCACH1A_PEA_1_T2 (SEQ ID NO: 707) | 3886 | 4087 |
| HUMCACH1A_PEA_1_T3 (SEQ ID NO: 708) | 3886 | 4087 |
| HUMCACH1A_PEA_1_T4 (SEQ ID NO: 709) | 3886 | 4087 |
| HUMCACH1A_PEA_1_T6 (SEQ ID NO: 710) | 3886 | 4087 |
| HUMCACH1A_PEA_1_T7 (SEQ ID NO: 711) | 3886 | 4087 |
| HUMCACH1A_PEA_1_T8 (SEQ ID NO: 712) | 3826 | 4027 |
| HUMCACH1A_PEA_1_T12 (SEQ ID NO: 713) | 2219 | 2420 |
| HUMCACH1A_PEA_1_T13 (SEQ ID NO: 714) | 3886 | 4087 |
| HUMCACH1A_PEA_1_T14 (SEQ ID NO: 715) | 3886 | 4087 |
| HUMCACH1A_PEA_1_T15 (SEQ ID NO: 716) | 3886 | 4087 |

Segment cluster HUMCACH1A_PEA_{—}1_node_{—}74 (SEQ ID NO:740) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T15 (SEQ ID NO:716). Table 59 below describes the starting and ending position of this segment on each transcript.

TABLE 59
Segment location on transcripts

| | Seg- | |
|---|---|---|
| | ment | Segment |
| | starting | ending |
| Transcript name | position | position |
| HUMCACH1A_PEA_1_T0 (SEQ ID NO: 705) | 4088 | 4246 |
| HUMCACH1A_PEA_1_T1 (SEQ ID NO: 706) | 3665 | 3823 |
| HUMCACH1A_PEA_1_T2 (SEQ ID NO: 707) | 4088 | 4246 |
| HUMCACH1A_PEA_1_T3 (SEQ ID NO: 708) | 4088 | 4246 |
| HUMCACH1A_PEA_1_T4 (SEQ ID NO: 709) | 4088 | 4246 |
| HUMCACH1A_PEA_1_T6 (SEQ ID NO: 710) | 4088 | 4246 |
| HUMCACH1A_PEA_1_T7 (SEQ ID NO: 711) | 4088 | 4246 |
| HUMCACH1A_PEA_1_T8 (SEQ ID NO: 712) | 4028 | 4186 |
| HUMCACH1A_PEA_1_T12 (SEQ ID NO: 713) | 2421 | 2579 |
| HUMCACH1A_PEA_1_T13 (SEQ ID NO: 714) | 4088 | 4246 |
| HUMCACH1A_PEA_1_T14 (SEQ ID NO: 715) | 4088 | 4246 |
| HUMCACH1A_PEA_1_T15 (SEQ ID NO: 716) | 4088 | 4246 |

Segment cluster HUMCACH1A_PEA_{—}1_node_{—}86 (SEQ ID NO:741) according to the present invention is supported by 1 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T17 (SEQ ID NO:718). Table 60 below describes the starting and ending position of this segment on each transcript.

TABLE 60
Segment location on transcripts

| | Seg- | |
|---|---|---|
| | ment | Segment |
| | starting | ending |
| Transcript name | position | position |
| HUMCACH1A_PEA_1_T0 (SEQ ID NO: 705) | 4487 | 4615 |
| HUMCACH1A_PEA_1_T1 (SEQ ID NO: 706) | 4064 | 4192 |
| HUMCACH1A_PEA_1_T2 (SEQ ID NO: 707) | 4487 | 4615 |
| HUMCACH1A_PEA_1_T3 (SEQ ID NO: 708) | 4487 | 4615 |
| HUMCACH1A_PEA_1_T4 (SEQ ID NO: 709) | 4487 | 4615 |
| HUMCACH1A_PEA_1_T6 (SEQ ID NO: 710) | 4487 | 4615 |
| HUMCACH1A_PEA_1_T7 (SEQ ID NO: 711) | 4487 | 4615 |
| HUMCACH1A_PEA_1_T8 (SEQ ID NO: 712) | 4427 | 4555 |
| HUMCACH1A_PEA_1_T12 (SEQ ID NO: 713) | 2820 | 2948 |
| HUMCACH1A_PEA_1_T13 (SEQ ID NO: 714) | 4487 | 4615 |
| HUMCACH1A_PEA_1_T14 (SEQ ID NO: 715) | 4487 | 4615 |
| HUMCACH1A_PEA_1_T17 (SEQ ID NO: 718) | 77 | 205 |

Segment cluster HUMCACH1A_PEA_{—}1_node_{—}92 (SEQ ID NO:742) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T17 (SEQ ID NO:718). Table 61 below describes the starting and ending position of this segment on each transcript.

TABLE 61
Segment location on transcripts

| | Seg- | |
|---|---|---|
| | ment | Segment |
| | starting | ending |
| Transcript name | position | position |
| HUMCACH1A_PEA_1_T0 (SEQ ID NO: 705) | 4774 | 4933 |
| HUMCACH1A_PEA_1_T1 (SEQ ID NO: 706) | 4351 | 4510 |
| HUMCACH1A_PEA_1_T2 (SEQ ID NO: 707) | 4774 | 4933 |
| HUMCACH1A_PEA_1_T3 (SEQ ID NO: 708) | 4774 | 4933 |
| HUMCACH1A_PEA_1_T4 (SEQ ID NO: 709) | 4774 | 4933 |
| HUMCACH1A_PEA_1_T6 (SEQ ID NO: 710) | 4774 | 4933 |
| HUMCACH1A_PEA_1_T7 (SEQ ID NO: 711) | 4774 | 4933 |
| HUMCACH1A_PEA_1_T8 (SEQ ID NO: 712) | 4714 | 4873 |
| HUMCACH1A_PEA_1_T12 (SEQ ID NO: 713) | 3107 | 3266 |
| HUMCACH1A_PEA_1_T13 (SEQ ID NO: 714) | 4774 | 4933 |
| HUMCACH1A_PEA_1_T14 (SEQ ID NO: 715) | 4774 | 4933 |
| HUMCACH1A_PEA_1_T17 (SEQ ID NO: 718) | 364 | 523 |

Segment cluster HUMCACH1A_PEA_{—}1_node_{—}94 (SEQ ID NO:743) according to the present invention is supported by 4 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T17 (SEQ ID NO:718). Table 62 below describes the starting and ending position of this segment on each transcript.

TABLE 62
Segment location on transcripts

| | Seg- | |
|---|---|---|
| | ment | Segment |
| | starting | ending |
| Transcript name | position | position |
| HUMCACH1A_PEA_1_T0 (SEQ ID NO: 705) | 4934 | 5061 |
| HUMCACH1A_PEA_1_T1 (SEQ ID NO: 706) | 4511 | 4638 |
| HUMCACH1A_PEA_1_T2 (SEQ ID NO: 707) | 4934 | 5061 |
| HUMCACH1A_PEA_1_T3 (SEQ ID NO: 708) | 4934 | 5061 |
| HUMCACH1A_PEA_1_T4 (SEQ ID NO: 709) | 4934 | 5061 |
| HUMCACH1A_PEA_1_T6 (SEQ ID NO: 710) | 4934 | 5061 |
| HUMCACH1A_PEA_1_T7 (SEQ ID NO: 711) | 4934 | 5061 |
| HUMCACH1A_PEA_1_T8 (SEQ ID NO: 712) | 4874 | 5001 |
| HUMCACH1A_PEA_1_T12 (SEQ ID NO: 713) | 3267 | 3394 |
| HUMCACH1A_PEA_1_T13 (SEQ ID NO: 714) | 4934 | 5061 |
| HUMCACH1A_PEA_1_T14 (SEQ ID NO: 715) | 4934 | 5061 |
| HUMCACH1A_PEA_1_T17 (SEQ ID NO: 718) | 524 | 651 |

Segment cluster HUMCACH1A_PEA_{—}1_node_{—}103 (SEQ ID NO:744) according to the present invention is supported by 2 libraries. The number of libraries was determined as previously described. This segment can be found in the following transcript(s): HUMCACH1A_PEA_{—}1_T18 (SEQ ID NO:719). Table 63 below describes the starting and ending position of this segment on each transcript.

TABLE 63
Segment locationon transcripts

| | Segment | Segment |
|---|---|---|
| | starting | ending |
| Transcript name | position | position |
| HUMCACH1A_PEA_1_T18 | 1 | 204 |
| (SEQ ID NO: 719) | | |

Segment cluster HUMCACH1A_PEA_{—}1_node_{—}104 (SEQ ID NO:745) according to the present invention is supported by 3 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T17 (SEQ ID NO:718) and HUMCACH1A_PEA_{—}1_T18 (SEQ ID NO:719). Table 64 below describes the starting and ending position of this segment on each transcript.

TABLE 64
Segment location on transcripts

| | Seg- | |
|---|---|---|
| | ment | Segment |
| | starting | ending |
| Transcript name | position | position |
| HUMCACH1A_PEA_1_T0 (SEQ ID NO: 705) | 5364 | 5494 |
| HUMCACH1A_PEA_1_T1 (SEQ ID NO: 706) | 4941 | 5071 |
| HUMCACH1A_PEA_1_T2 (SEQ ID NO: 707) | 5364 | 5494 |
| HUMCACH1A_PEA_1_T3 (SEQ ID NO: 708) | 5364 | 5494 |
| HUMCACH1A_PEA_1_T4 (SEQ ID NO: 709) | 5364 | 5494 |
| HUMCACH1A_PEA_1_T6 (SEQ ID NO: 710) | 5364 | 5494 |
| HUMCACH1A_PEA_1_T7 (SEQ ID NO: 711) | 5364 | 5494 |
| HUMCACH1A_PEA_1_T8 (SEQ ID NO: 712) | 5304 | 5434 |
| HUMCACH1A_PEA_1_T12 (SEQ ID NO: 713) | 3697 | 3827 |
| HUMCACH1A_PEA_1_T13 (SEQ ID NO: 714) | 5364 | 5494 |
| HUMCACH1A_PEA_1_T17 (SEQ ID NO: 718) | 954 | 1084 |
| HUMCACH1A_PEA_1_T18 (SEQ ID NO: 719) | 205 | 335 |

Segment cluster HUMCACH1A_PEA_{—}1_node_{—}106 (SEQ ID NO:746) according to the present invention is supported by 4 libraries. The number of libraries was determined as previously described. This segment can be found in the following transcript(s): HUMCACH1A_PEA_{—}1_T19 (SEQ ID NO:720). Table 65 below describes the starting and ending position of this segment on each transcript.

TABLE 65
Segment location on transcripts

| | Segment | Segment |
|---|---|---|
| | starting | ending |
| Transcript name | position | position |
| HUMCACH1A_PEA_1_T19 | 1 | 1456 |
| (SEQ ID NO: 720) | | |

Segment cluster HUMCACH1A_PEA_{—}1_node_{—}109 (SEQ ID NO:747) according to the present invention is supported by 6 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T17 (SEQ ID NO:718), HUMCACH1A_PEA_{—}1_T18 (SEQ ID NO:719) and HUMCACH1A_PEA_{—}1_T19 (SEQ ID NO:720). Table 66 below describes the starting and ending position of this segment on each transcript.

TABLE 66
Segment location on transcripts

| | Seg- | |
|---|---|---|
| | ment | Segment |
| | starting | ending |
| Transcript name | position | position |
| HUMCACH1A_PEA_1_T0 (SEQ ID NO: 705) | 5612 | 5979 |
| HUMCACH1A_PEA_1_T1 (SEQ ID NO: 706) | 5189 | 5556 |
| HUMCACH1A_PEA_1_T2 (SEQ ID NO: 707) | 5612 | 5979 |
| HUMCACH1A_PEA_1_T3 (SEQ ID NO: 708) | 5612 | 5979 |
| HUMCACH1A_PEA_1_T4 (SEQ ID NO: 709) | 5612 | 5979 |
| HUMCACH1A_PEA_1_T6 (SEQ ID NO: 710) | 5612 | 5979 |
| HUMCACH1A_PEA_1_T7 (SEQ ID NO: 711) | 5612 | 5979 |
| HUMCACH1A_PEA_1_T8 (SEQ ID NO: 712) | 5552 | 5919 |
| HUMCACH1A_PEA_1_T12 (SEQ ID NO: 713) | 3945 | 4312 |
| HUMCACH1A_PEA_1_T13 (SEQ ID NO: 714) | 5612 | 5979 |
| HUMCACH1A_PEA_1_T17 (SEQ ID NO: 718) | 1202 | 1569 |
| HUMCACH1A_PEA_1_T18 (SEQ ID NO: 719) | 453 | 820 |
| HUMCACH1A_PEA_1_T19 (SEQ ID NO: 720) | 1574 | 1941 |

Segment cluster HUMCACH1A_PEA_{—}1_node_{—}113 (SEQ ID NO:748) according to the present invention is supported by 4 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T17 (SEQ ID NO:718), HUMCACH1A_PEA_{—}1_T18 (SEQ ID NO:719) and HUMCACH1A_PEA_{—}1_T19 (SEQ ID NO:720). Table 67 below describes the starting and ending position of this segment on each transcript.

TABLE 67
Segment location on transcripts

| | Seg- | |
|---|---|---|
| | ment | Segment |
| | starting | ending |
| Transcript name | position | position |
| HUMCACH1A_PEA_1_T0 (SEQ ID NO: 705) | 6007 | 6156 |
| HUMCACH1A_PEA_1_T1 (SEQ ID NO: 706) | 5584 | 5733 |
| HUMCACH1A_PEA_1_T2 (SEQ ID NO: 707) | 6007 | 6156 |
| HUMCACH1A_PEA_1_T3 (SEQ ID NO: 708) | 6007 | 6156 |
| HUMCACH1A_PEA_1_T4 (SEQ ID NO: 709) | 6007 | 6156 |
| HUMCACH1A_PEA_1_T6 (SEQ ID NO: 710) | 6007 | 6156 |
| HUMCACH1A_PEA_1_T7 (SEQ ID NO: 711) | 5980 | 6129 |
| HUMCACH1A_PEA_1_T8 (SEQ ID NO: 712) | 5947 | 6096 |
| HUMCACH1A_PEA_1_T12 (SEQ ID NO: 713) | 4340 | 4489 |
| HUMCACH1A_PEA_1_T13 (SEQ ID NO: 714) | 5980 | 6129 |
| HUMCACH1A_PEA_1_T17 (SEQ ID NO: 718) | 1597 | 1746 |
| HUMCACH1A_PEA_1_T18 (SEQ ID NO: 719) | 848 | 997 |
| HUMCACH1A_PEA_1_T19 (SEQ ID NO: 720) | 1969 | 2118 |

Segment cluster HUMCACH1A_PEA_{—}1_node_{—}114 (SEQ ID NO:749) according to the present invention is supported by 1 libraries. The number of libraries was determined as previously described. This segment can be found in the following transcript(s): HUMCACH1A_PEA_{—}1_T6 (SEQ ID NO:710). Table 68 below describes the starting and ending position of this segment on each transcript.

TABLE 68
Segment location on transcripts

| | Segment | Segment |
|---|---|---|
| | starting | ending |
| Transcript name | position | position |
| HUMCACH1A_PEA_1_T6 | 6157 | 6335 |
| (SEQ ID NO: 710) | | |

Segment cluster HUMCACH1A_PEA_{—}1_node_{—}116 (SEQ ID NO:750) according to the present invention is supported by 4 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T17 (SEQ ID NO:718), HUMCACH1A_PEA_{—}1_T18 (SEQ ID NO:719) and HUMCACH1A_PEA_{—}1_T19 (SEQ ID NO:720). Table 69 below describes the starting and ending position of this segment on each transcript.

TABLE 69
Segment location on transcripts

| | Seg- | |
|---|---|---|
| | ment | Segment |
| | starting | ending |
| Transcript name | position | position |
| HUMCACH1A_PEA_1_T0 (SEQ ID NO: 705) | 6157 | 6320 |
| HUMCACH1A_PEA_1_T1 (SEQ ID NO: 706) | 5734 | 5897 |
| HUMCACH1A_PEA_1_T2 (SEQ ID NO: 707) | 6157 | 6320 |
| HUMCACH1A_PEA_1_T3 (SEQ ID NO: 708) | 6157 | 6320 |
| HUMCACH1A_PEA_1_T4 (SEQ ID NO: 709) | 6157 | 6320 |
| HUMCACH1A_PEA_1_T6 (SEQ ID NO: 710) | 6336 | 6499 |
| HUMCACH1A_PEA_1_T7 (SEQ ID NO: 711) | 6130 | 6293 |
| HUMCACH1A_PEA_1_T8 (SEQ ID NO: 712) | 6097 | 6260 |
| HUMCACH1A_PEA_1_T12 (SEQ ID NO: 713) | 4490 | 4653 |
| HUMCACH1A_PEA_1_T13 (SEQ ID NO: 714) | 6130 | 6293 |
| HUMCACH1A_PEA_1_T17 (SEQ ID NO: 718) | 1747 | 1910 |
| HUMCACH1A_PEA_1_T18 (SEQ ID NO: 719) | 998 | 1161 |
| HUMCACH1A_PEA_1_T19 (SEQ ID NO: 720) | 2119 | 2282 |

Segment cluster HUMCACH1A_PEA_{—}1_node_{—}119 (SEQ ID NO:751) according to the present invention is supported by 4 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T17 (SEQ ID NO:718), HUMCACH1A_PEA_{—}1_T18 (SEQ ID NO:719) and HUMCACH1A_PEA_{—}1_T19 (SEQ ID NO:720). Table 70 below describes the starting and ending position of this segment on each transcript.

TABLE 70
Segment location on transcripts

| | Seg- | |
|---|---|---|
| | ment | Segment |
| | starting | ending |
| Transcript name | position | position |
| HUMCACH1A_PEA_1_T0 (SEQ ID NO: 705) | 6321 | 6442 |
| HUMCACH1A_PEA_1_T1 (SEQ ID NO: 706) | 5898 | 6019 |
| HUMCACH1A_PEA_1_T2 (SEQ ID NO: 707) | 6321 | 6442 |
| HUMCACH1A_PEA_1_T3 (SEQ ID NO: 708) | 6321 | 6442 |
| HUMCACH1A_PEA_1_T4 (SEQ ID NO: 709) | 6321 | 6442 |
| HUMCACH1A_PEA_1_T6 (SEQ ID NO: 710) | 6500 | 6621 |
| HUMCACH1A_PEA_1_T7 (SEQ ID NO: 711) | 6294 | 6415 |
| HUMCACH1A_PEA_1_T8 (SEQ ID NO: 712) | 6261 | 6382 |
| HUMCACH1A_PEA_1_T12 (SEQ ID NO: 713) | 4654 | 4775 |
| HUMCACH1A_PEA_1_T17 (SEQ ID NO: 718) | 1911 | 2032 |
| HUMCACH1A_PEA_1_T18 (SEQ ID NO: 719) | 1162 | 1283 |
| HUMCACH1A_PEA_1_T19 (SEQ ID NO: 720) | 2283 | 2404 |

Segment cluster HUMCACH1A_PEA_{—}1_node_{—}12 (SEQ ID NO:752) according to the present invention is supported by 15 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T17 (SEQ ID NO:718), HUMCACH1A_PEA_{—}1_T18 (SEQ ID NO:719) and HUMCACH1A_PEA_{—}1_T19 (SEQ ID NO:720). Table 71 below describes the starting and ending position of this segment on each transcript.

TABLE 71
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 6443 | 6763 |
| HUMCACH1A_PEA_1_T1 (SEQ ID NO: 706) | 6020 | 6340 |
| HUMCACH1A_PEA_1_T2 (SEQ ID NO: 707) | 6443 | 6763 |
| HUMCACH1A_PEA_1_T3 (SEQ ID NO: 708) | 6443 | 6763 |
| HUMCACH1A_PEA_1_T4 (SEQ ID NO: 709) | 6443 | 6763 |
| HUMCACH1A_PEA_1_T6 (SEQ ID NO: 710) | 6622 | 6942 |
| HUMCACH1A_PEA_1_T7 (SEQ ID NO: 711) | 6416 | 6736 |
| HUMCACH1A_PEA_1_T8 (SEQ ID NO: 712) | 6383 | 6703 |
| HUMCACH1A_PEA_1_T12 (SEQ ID NO: 713) | 4776 | 5096 |
| HUMCACH1A_PEA_1_T17 (SEQ ID NO: 718) | 2033 | 2353 |
| HUMCACH1A_PEA_1_T18 (SEQ ID NO: 719) | 1284 | 1604 |
| HUMCACH1A_PEA_1_T19 (SEQ ID NO: 720) | 2405 | 2725 |

Segment cluster HUMCACH1A_PEA_{—}1_node_{—}123 (SEQ ID NO:753) according to the present invention is supported by 28 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T17 (SEQ ID NO:718), HUMCACH1A_PEA_{—}1_T18 (SEQ ID NO:719) and HUMCACH1A_PEA_{—}1_T19 (SEQ ID NO:720). Table 72 below describes the starting and ending position of this segment on each transcript.

TABLE 72
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 6764 | 7550 |
| HUMCACH1A_PEA_1_T1 (SEQ ID NO: 706) | 6341 | 7127 |
| HUMCACH1A_PEA_1_T2 (SEQ ID NO: 707) | 6764 | 7550 |
| HUMCACH1A_PEA_1_T3 (SEQ ID NO: 708) | 6764 | 7550 |
| HUMCACH1A_PEA_1_T4 (SEQ ID NO: 709) | 6764 | 7550 |
| HUMCACH1A_PEA_1_T6 (SEQ ID NO: 710) | 6943 | 7729 |
| HUMCACH1A_PEA_1_T7 (SEQ ID NO: 711) | 6737 | 7523 |
| HUMCACH1A_PEA_1_T8 (SEQ ID NO: 712) | 6704 | 7490 |
| HUMCACH1A_PEA_1_T12 (SEQ ID NO: 713) | 5097 | 5883 |
| HUMCACH1A_PEA_1_T17 (SEQ ID NO: 718) | 2354 | 3140 |
| HUMCACH1A_PEA_1_T18 (SEQ ID NO: 719) | 1605 | 2391 |
| HUMCACH1A_PEA_1_T19 (SEQ ID NO: 720) | 2726 | 3512 |

Segment cluster HUMCACH1A_PEA_{—}1_node_{—}125 (SEQ ID NO:754) according to the present invention is supported by 48 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T17 (SEQ ID NO:718), HUMCACH1A_PEA_{—}1_T18 (SEQ ID NO:719) and HUMCACH1A_PEA_{—}1_T19 (SEQ ID NO:720). Table 73 below describes the starting and ending position of this segment on each transcript.

TABLE 73
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 7650 | 9310 |
| HUMCACH1A_PEA_1_T1 (SEQ ID NO: 706) | 7227 | 8887 |
| HUMCACH1A_PEA_1_T2 (SEQ ID NO: 707) | 7650 | 9310 |
| HUMCACH1A_PEA_1_T3 (SEQ ID NO: 708) | 7650 | 8114 |
| HUMCACH1A_PEA_1_T4 (SEQ ID NO: 709) | 7650 | 7850 |
| HUMCACH1A_PEA_1_T6 (SEQ ID NO: 710) | 7829 | 9489 |
| HUMCACH1A_PEA_1_T7 (SEQ ID NO: 711) | 7623 | 9283 |
| HUMCACH1A_PEA_1_T8 (SEQ ID NO: 712) | 7590 | 9250 |
| HUMCACH1A_PEA_1_T12 (SEQ ID NO: 713) | 5983 | 7643 |
| HUMCACH1A_PEA_1_T17 (SEQ ID NO: 718) | 3240 | 4900 |
| HUMCACH1A_PEA_1_T18 (SEQ ID NO: 719) | 2491 | 4151 |
| HUMCACH1A_PEA_1_T19 (SEQ ID NO: 720) | 3612 | 5272 |

Segment cluster HUMCACH1A_PEA_{—}1_node_{—}128 (SEQ ID NO:755) according to the present invention is supported by 1 libraries. The number of libraries was determined as previously described. This segment can be found in the following transcript(s): HUMCACH1A_PEA_{—}1_T2 (SEQ ID NO:707). Table 74 below describes the starting and ending position of this segment on each transcript.

TABLE 74
Segment location on transcripts

| | Segment | Segment |
|---|---|---|
| Transcript name | starting position | ending position |
| HUMCACH1A_PEA_1_T2 | 9311 | 9458 |
| (SEQ ID NO: 707) | | |

According to an optional embodiment of the present invention, short segments related to the above cluster are also provided. These segments are up to about 120 bp in length, and so are included in a separate description.

Segment cluster HUMCACH1A_PEA_{—}1_node_{—}0 (SEQ ID NO:756) according to the present invention is supported by 1 libraries. The number of libraries was determined as previously described. This segment can be found in the following transcript(s): HUMCACH1A_PEA_{—}1_T1 (SEQ ID NO:706). Table 75 below describes the starting and ending position of this segment on each transcript.

TABLE 75
Segment location on transcripts

| | Segment | Segment |
|---|---|---|
| Transcript name | starting position | ending position |
| HUMCACH1A_PEA_1_T1 | 1 | 45 |
| (SEQ ID NO: 706) | | |

Segment cluster HUMCACH1A_PEA_{—}1_node_{—}3 (SEQ ID NO:757) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716), HUMCACH1A_PEA_{—}1_T16 (SEQ ID NO:717), HUMCACH1A_PEA_{—}1_T20 (SEQ ID NO:721) and HUMCACH1A_PEA_{—}1_T22 (SEQ ID NO:722). Table 76 below describes the starting and ending position of this segment on each transcript.

TABLE 76
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 469 | 578 |
| HUMCACH1A_PEA_1_T1 (SEQ ID NO: 706) | 46 | 155 |
| HUMCACH1A_PEA_1_T2 (SEQ ID NO: 707) | 469 | 578 |
| HUMCACH1A_PEA_1_T3 (SEQ ID NO: 708) | 469 | 578 |
| HUMCACH1A_PEA_1_T4 (SEQ ID NO: 709) | 469 | 578 |
| HUMCACH1A_PEA_1_T6 (SEQ ID NO: 710) | 469 | 578 |
| HUMCACH1A_PEA_1_T7 (SEQ ID NO: 711) | 469 | 578 |
| HUMCACH1A_PEA_1_T8 (SEQ ID NO: 712) | 469 | 578 |
| HUMCACH1A_PEA_1_T13 (SEQ ID NO: 714) | 469 | 578 |
| HUMCACH1A_PEA_1_T14 (SEQ ID NO: 715) | 469 | 578 |
| HUMCACH1A_PEA_1_T15 (SEQ ID NO: 716) | 469 | 578 |
| HUMCACH1A_PEA_1_T16 (SEQ ID NO: 717) | 469 | 578 |
| HUMCACH1A_PEA_1_T20 (SEQ ID NO: 721) | 469 | 578 |
| HUMCACH1A_PEA_1_T22 (SEQ ID NO: 722) | 469 | 578 |

Segment cluster HUMCACH1A_PEA_{—}1_node_{—}7 (SEQ ID NO:758) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716), HUMCACH1A_PEA_{—}1_T16 (SEQ ID NO:717), HUMCACH1A_PEA_{—}1_T20 (SEQ ID NO:721) and HUMCACH1A_PEA_{—}1_T22 (SEQ ID NO:722). Table 77 below describes the starting and ending position of this segment on each transcript.

TABLE 77
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 889 | 994 |
| HUMCACH1A_PEA_1_T1 (SEQ ID NO: 706) | 466 | 571 |
| HUMCACH1A_PEA_1_T2 (SEQ ID NO: 707) | 889 | 994 |
| HUMCACH1A_PEA_1_T3 (SEQ ID NO: 708) | 889 | 994 |
| HUMCACH1A_PEA_1_T4 (SEQ ID NO: 709) | 889 | 994 |
| HUMCACH1A_PEA_1_T6 (SEQ ID NO: 710) | 889 | 994 |
| HUMCACH1A_PEA_1_T7 (SEQ ID NO: 711) | 889 | 994 |
| HUMCACH1A_PEA_1_T8 (SEQ ID NO: 712) | 889 | 994 |
| HUMCACH1A_PEA_1_T13 (SEQ ID NO: 714) | 889 | 994 |
| HUMCACH1A_PEA_1_T14 (SEQ ID NO: 715) | 889 | 994 |
| HUMCACH1A_PEA_1_T15 (SEQ ID NO: 716) | 889 | 994 |
| HUMCACH1A_PEA_1_T16 (SEQ ID NO: 717) | 889 | 994 |
| HUMCACH1A_PEA_1_T20 (SEQ ID NO: 721) | 889 | 994 |
| HUMCACH1A_PEA_1_T22 (SEQ ID NO: 722) | 889 | 994 |

Segment cluster HUMCACH1A_PEA_{—}1_node_{—}23 (SEQ ID NO:759) according to the present invention is supported by 5 libraries. The number of libraries was determined as previously described. This segment can be found in the following transcript(s): HUMCACH1A_PEA_{—}1_T8 (SEQ ID NO:712) and HUMCACH1A_PEA_{—}1_T22 (SEQ ID NO:722). Table 78 below describes the starting and ending position of this segment on each transcript.

TABLE 78
Segment location on transcripts

| | Segment | Segment |
|---|---|---|
| Transcript name | starting position | ending position |
| HUMCACH1A_PEA_1_T8 | 1628 | 1731 |
| (SEQ ID NO: 712) | | |
| HUMCACH1A_PEA_1_T22 | 1628 | 1731 |
| (SEQ ID NO: 722) | | |

Segment cluster HUMCACH1A_PEA_{—}1_node_{—}26 (SEQ ID NO:760) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716), HUMCACH1A_PEA_{—}1_T16 (SEQ ID NO:717) and HUMCACH1A_PEA_{—}1_T20 (SEQ ID NO:721). Table 79 below describes the starting and ending position of this segment on each transcript.

TABLE 79
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 1628 | 1731 |
| HUMCACH1A_PEA_1_T1 (SEQ ID NO: 706) | 1205 | 1308 |
| HUMCACH1A_PEA_1_T2 (SEQ ID NO: 707) | 1628 | 1731 |
| HUMCACH1A_PEA_1_T3 (SEQ ID NO: 708) | 1628 | 1731 |
| HUMCACH1A_PEA_1_T4 (SEQ ID NO: 709) | 1628 | 1731 |
| HUMCACH1A_PEA_1_T6 (SEQ ID NO: 710) | 1628 | 1731 |
| HUMCACH1A_PEA_1_T7 (SEQ ID NO: 711) | 1628 | 1731 |
| HUMCACH1A_PEA_1_T13 (SEQ ID NO: 714) | 1628 | 1731 |
| HUMCACH1A_PEA_1_T14 (SEQ ID NO: 715) | 1628 | 1731 |
| HUMCACH1A_PEA_1_T15 (SEQ ID NO: 716) | 1628 | 1731 |
| HUMCACH1A_PEA_1_T16 (SEQ ID NO: 717) | 1628 | 1731 |
| HUMCACH1A_PEA_1_T20 (SEQ ID NO: 721) | 1628 | 1731 |

Segment cluster HUMCACH1A_PEA_{—}1_node_{—}32 (SEQ ID NO:761) according to the present invention is supported by 2 libraries. The number of libraries was determined as previously described. This segment can be found in the following transcript(s): HUMCACH1A_PEA_{—}1_T12 (SEQ ID NO:713). Table 80 below describes the starting and ending position of this segment on each transcript.

TABLE 80
Segment location on transcripts

| | Segment | Segment |
|---|---|---|
| Transcript name | starting position | ending position |
| HUMCACH1A_PEA_1_T12 | 1 | 64 |
| (SEQ ID NO: 713) | | |

Segment cluster HUMCACH1A_PEA_{—}1_node_{—}35 (SEQ ID NO:762) according to the present invention is supported by 1 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716) and HUMCACH1A_PEA_{—}1_T16 (SEQ ID NO:717). Table 81 below describes the starting and ending position of this segment on each transcript.

TABLE 81
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 1902 | 1989 |
| HUMCACH1A_PEA_1_T1 (SEQ ID NO: 706) | 1479 | 1566 |
| HUMCACH1A_PEA_1_T2 (SEQ ID NO: 707) | 1902 | 1989 |
| HUMCACH1A_PEA_1_T3 (SEQ ID NO: 708) | 1902 | 1989 |
| HUMCACH1A_PEA_1_T4 (SEQ ID NO: 709) | 1902 | 1989 |
| HUMCACH1A_PEA_1_T6 (SEQ ID NO: 710) | 1902 | 1989 |
| HUMCACH1A_PEA_1_T7 (SEQ ID NO: 711) | 1902 | 1989 |
| HUMCACH1A_PEA_1_T8 (SEQ ID NO: 712) | 1902 | 1989 |
| HUMCACH1A_PEA_1_T12 (SEQ ID NO: 713) | 235 | 322 |
| HUMCACH1A_PEA_1_T13 (SEQ ID NO: 714) | 1902 | 1989 |
| HUMCACH1A_PEA_1_T14 (SEQ ID NO: 715) | 1902 | 1989 |
| HUMCACH1A_PEA_1_T15 (SEQ ID NO: 716) | 1902 | 1989 |
| HUMCACH1A_PEA_1_T16 (SEQ ID NO: 717) | 1902 | 1989 |

Segment cluster HUMCACH1A_PEA_{—}1_node_{—}37 (SEQ ID NO:763) according to the present invention is supported by 1 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716) and HUMCACH1A_PEA_{—}1_T16 (SEQ ID NO:717). Table 82 below describes the starting and ending position of this segment on each transcript.

TABLE 82
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 1990 | 2049 |
| HUMCACH1A_PEA_1_T1 (SEQ ID NO: 706) | 1567 | 1626 |
| HUMCACH1A_PEA_1_T2 (SEQ ID NO: 707) | 1990 | 2049 |
| HUMCACH1A_PEA_1_T3 (SEQ ID NO: 708) | 1990 | 2049 |
| HUMCACH1A_PEA_1_T4 (SEQ ID NO: 709) | 1990 | 2049 |
| HUMCACH1A_PEA_1_T6 (SEQ ID NO: 710) | 1990 | 2049 |
| HUMCACH1A_PEA_1_T7 (SEQ ID NO: 711) | 1990 | 2049 |
| HUMCACH1A_PEA_1_T12 (SEQ ID NO: 713) | 323 | 382 |
| HUMCACH1A_PEA_1_T13 (SEQ ID NO: 714) | 1990 | 2049 |
| HUMCACH1A_PEA_1_T14 (SEQ ID NO: 715) | 1990 | 2049 |
| HUMCACH1A_PEA_1_T15 (SEQ ID NO: 716) | 1990 | 2049 |
| HUMCACH1A_PEA_1_T16 (SEQ ID NO: 717) | 1990 | 2049 |

Segment cluster HUMCACH1A_PEA_{—}1_node_{—}39 (SEQ ID NO:764) according to the present invention is supported by 4 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716) and HUMCACH1A_PEA_{—}1_T16 (SEQ ID NO:717). Table 83 below describes the starting and ending position of this segment on each transcript.

TABLE 83
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 2050 | 2076 |
| HUMCACH1A_PEA_1_T1 (SEQ ID NO: 706) | 1627 | 1653 |
| HUMCACH1A_PEA_1_T2 (SEQ ID NO: 707) | 2050 | 2076 |
| HUMCACH1A_PEA_1_T3 (SEQ ID NO: 708) | 2050 | 2076 |
| HUMCACH1A_PEA_1_T4 (SEQ ID NO: 709) | 2050 | 2076 |
| HUMCACH1A_PEA_1_T6 (SEQ ID NO: 710) | 2050 | 2076 |
| HUMCACH1A_PEA_1_T7 (SEQ ID NO: 711) | 2050 | 2076 |
| HUMCACH1A_PEA_1_T8 (SEQ ID NO: 712) | 1990 | 2016 |
| HUMCACH1A_PEA_1_T12 (SEQ ID NO: 713) | 383 | 409 |
| HUMCACH1A_PEA_1_T13 (SEQ ID NO: 714) | 2050 | 2076 |
| HUMCACH1A_PEA_1_T14 (SEQ ID NO: 715) | 2050 | 2076 |
| HUMCACH1A_PEA_1_T15 (SEQ ID NO: 716) | 2050 | 2076 |
| HUMCACH1A_PEA_1_T16 (SEQ ID NO: 717) | 2050 | 2076 |

Segment cluster HUMCACH1A_PEA_{—}1_node_{—}49 (SEQ ID NO:765) according to the present invention is supported by 4 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716) and HUMCACH1A_PEA_{—}1_T16 (SEQ ID NO:717). Table 84 below describes the starting and ending position of this segment on each transcript.

TABLE 84
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 2793 | 2907 |
| HUMCACH1A_PEA_1_T1 (SEQ ID NO: 706) | 2370 | 2484 |
| HUMCACH1A_PEA_1_T2 (SEQ ID NO: 707) | 2793 | 2907 |
| HUMCACH1A_PEA_1_T3 (SEQ ID NO: 708) | 2793 | 2907 |
| HUMCACH1A_PEA_1_T4 (SEQ ID NO: 709) | 2793 | 2907 |
| HUMCACH1A_PEA_1_T6 (SEQ ID NO: 710) | 2793 | 2907 |
| HUMCACH1A_PEA_1_T7 (SEQ ID NO: 711) | 2793 | 2907 |
| HUMCACH1A_PEA_1_T8 (SEQ ID NO: 712) | 2733 | 2847 |
| HUMCACH1A_PEA_1_T12 (SEQ ID NO: 713) | 1126 | 1240 |
| HUMCACH1A_PEA_1_T13 (SEQ ID NO: 714) | 2793 | 2907 |
| HUMCACH1A_PEA_1_T14 (SEQ ID NO: 715) | 2793 | 2907 |
| HUMCACH1A_PEA_1_T15 (SEQ ID NO: 716) | 2793 | 2907 |
| HUMCACH1A_PEA_1_T16 (SEQ ID NO: 717) | 2793 | 2907 |

Segment cluster HUMCACH1A_PEA_{—}1_node_{—}51 (SEQ ID NO:766) according to the present invention is supported by 4 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716) and HUMCACH1A_PEA_{—}1_T16 (SEQ ID NO:717). Table 85 below describes the starting and ending position of this segment on each transcript.

TABLE 85
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 2908 | 2977 |
| HUMCACH1A_PEA_1_T1 (SEQ ID NO: 706) | 2485 | 2554 |
| HUMCACH1A_PEA_1_T2 (SEQ ID NO: 707) | 2908 | 2977 |
| HUMCACH1A_PEA_1_T3 (SEQ ID NO: 708) | 2908 | 2977 |
| HUMCACH1A_PEA_1_T4 (SEQ ID NO: 709) | 2908 | 2977 |
| HUMCACH1A_PEA_1_T6 (SEQ ID NO: 710) | 2908 | 2977 |
| HUMCACH1A_PEA_1_T7 (SEQ ID NO: 711) | 2908 | 2977 |
| HUMCACH1A_PEA_1_T8 (SEQ ID NO: 712) | 2848 | 2917 |
| HUMCACH1A_PEA_1_T12 (SEQ ID NO: 713) | 1241 | 1310 |
| HUMCACH1A_PEA_1_T13 (SEQ ID NO: 714) | 2908 | 2977 |
| HUMCACH1A_PEA_1_T14 (SEQ ID NO: 715) | 2908 | 2977 |
| HUMCACH1A_PEA_1_T15 (SEQ ID NO: 716) | 2908 | 2977 |
| HUMCACH1A_PEA_1_T16 (SEQ ID NO: 717) | 2908 | 2977 |

Segment cluster HUMCACH1A_PEA_{—}1_node_{—}53 (SEQ ID NO:767) according to the present invention is supported by 4 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715), HUMCACH1A_PEA_{—}1_T15 (SEQ ID NO:716) and HUMCACH1A_PEA_{—}1_T16 (SEQ ID NO:717). Table 86 below describes the starting and ending position of this segment on each transcript.

TABLE 86
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 2978 | 3044 |
| HUMCACH1A_PEA_1_T1 (SEQ ID NO: 706) | 2555 | 2621 |
| HUMCACH1A_PEA_1_T2 (SEQ ID NO: 707) | 2978 | 3044 |
| HUMCACH1A_PEA_1_T3 (SEQ ID NO: 708) | 2978 | 3044 |
| HUMCACH1A_PEA_1_T4 (SEQ ID NO: 709) | 2978 | 3044 |
| HUMCACH1A_PEA_1_T6 (SEQ ID NO: 710) | 2978 | 3044 |
| HUMCACH1A_PEA_1_T7 (SEQ ID NO: 711) | 2978 | 3044 |
| HUMCACH1A_PEA_1_T8 (SEQ ID NO: 712) | 2918 | 2984 |
| HUMCACH1A_PEA_1_T12 (SEQ ID NO: 713) | 1311 | 1377 |
| HUMCACH1A_PEA_1_T13 (SEQ ID NO: 714) | 2978 | 3044 |
| HUMCACH1A_PEA_1_T14 (SEQ ID NO: 715) | 2978 | 3044 |
| HUMCACH1A_PEA_1_T15 (SEQ ID NO: 716) | 2978 | 3044 |
| HUMCACH1A_PEA_1_T16 (SEQ ID NO: 717) | 2978 | 3044 |

Segment cluster HUMCACH1A_PEA_{—}1_node_{—}58 (SEQ ID NO:768) according to the present invention is supported by 1 libraries. The number of libraries was determined as previously described. This segment can be found in the following transcript(s): HUMCACH1A_PEA_{—}1_T16 (SEQ ID NO:717). Table 87 below describes the starting and ending position of this segment on each transcript.

TABLE 87
Segment location on transcripts

| | | Seg- |
|---|---|---|
| | Segment | ment |
| | starting | ending |
| Transcript name | position | position |
| HUMCACH1A_PEA_1_T16 (SEQ ID NO: 717) | 3323 | 3363 |

Segment cluster HUMCACH1A_PEA_{—}1_node_{—}60 (SEQ ID NO:769) according to the present invention is supported by 4 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T15 (SEQ ID NO:716). Table 88 below describes the starting and ending position of this segment on each transcript.

TABLE 88
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 3323 | 3382 |
| HUMCACH1A_PEA_1_T1 (SEQ ID NO: 706) | 2900 | 2959 |
| HUMCACH1A_PEA_1_T2 (SEQ ID NO: 707) | 3323 | 3382 |
| HUMCACH1A_PEA_1_T3 (SEQ ID NO: 708) | 3323 | 3382 |
| HUMCACH1A_PEA_1_T4 (SEQ ID NO: 709) | 3323 | 3382 |
| HUMCACH1A_PEA_1_T6 (SEQ ID NO: 710) | 3323 | 3382 |
| HUMCACH1A_PEA_1_T7 (SEQ ID NO: 711) | 3323 | 3382 |
| HUMCACH1A_PEA_1_T8 (SEQ ID NO: 712) | 3263 | 3322 |
| HUMCACH1A_PEA_1_T12 (SEQ ID NO: 713) | 1656 | 1715 |
| HUMCACH1A_PEA_1_T13 (SEQ ID NO: 714) | 3323 | 3382 |
| HUMCACH1A_PEA_1_T14 (SEQ ID NO: 715) | 3323 | 3382 |
| HUMCACH1A_PEA_1_T15 (SEQ ID NO: 716) | 3323 | 3382 |

Segment cluster HUMCACH1A_PEA_{—}1_node_{—}62 (SEQ ID NO:770) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T15 (SEQ ID NO:716). Table 89 below describes the starting and ending position of this segment on each transcript.

TABLE 89
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 3383 | 3489 |
| HUMCACH1A_PEA_1_T1 (SEQ ID NO: 706) | 2960 | 3066 |
| HUMCACH1A_PEA_1_T2 (SEQ ID NO: 707) | 3383 | 3489 |
| HUMCACH1A_PEA_1_T3 (SEQ ID NO: 708) | 3383 | 3489 |
| HUMCACH1A_PEA_1_T4 (SEQ ID NO: 709) | 3383 | 3489 |
| HUMCACH1A_PEA_1_T6 (SEQ ID NO: 710) | 3383 | 3489 |
| HUMCACH1A_PEA_1_T7 (SEQ ID NO: 711) | 3383 | 3489 |
| HUMCACH1A_PEA_1_T8 (SEQ ID NO: 712) | 3323 | 3429 |
| HUMCACH1A_PEA_1_T12 (SEQ ID NO: 713) | 1716 | 1822 |
| HUMCACH1A_PEA_1_T13 (SEQ ID NO: 714) | 3383 | 3489 |
| HUMCACH1A_PEA_1_T14 (SEQ ID NO: 715) | 3383 | 3489 |
| HUMCACH1A_PEA_1_T15 (SEQ ID NO: 716) | 3383 | 3489 |

Segment cluster HUMCACH1A_PEA_{—}1_node_{—}64 (SEQ ID NO:771) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T15 (SEQ ID NO:716). Table 90 below describes the starting and ending position of this segment on each transcript.

TABLE 90
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 3490 | 3577 |
| HUMCACH1A_PEA_1_T1 (SEQ ID NO: 706) | 3067 | 3154 |
| HUMCACH1A_PEA_1_T2 (SEQ ID NO: 707) | 3490 | 3577 |
| HUMCACH1A_PEA_1_T3 (SEQ ID NO: 708) | 3490 | 3577 |
| HUMCACH1A_PEA_1_T4 (SEQ ID NO: 709) | 3490 | 3577 |
| HUMCACH1A_PEA_1_T6 (SEQ ID NO: 710) | 3490 | 3577 |
| HUMCACH1A_PEA_1_T7 (SEQ ID NO: 711) | 3490 | 3577 |
| HUMCACH1A_PEA_1_T8 (SEQ ID NO: 712) | 3430 | 3517 |
| HUMCACH1A_PEA_1_T12 (SEQ ID NO: 713) | 1823 | 1910 |
| HUMCACH1A_PEA_1_T13 (SEQ ID NO: 714) | 3490 | 3577 |
| HUMCACH1A_PEA_1_T14 (SEQ ID NO: 715) | 3490 | 3577 |
| HUMCACH1A_PEA_1_T15 (SEQ ID NO: 716) | 3490 | 3577 |

Segment cluster HUMCACH1A_PEA_{—}1_node_{—}66 (SEQ ID NO:772) according to the present invention is supported by 4 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T15 (SEQ ID NO:716). Table 91 below describes the starting and ending position of this segment on each transcript.

TABLE 91
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 3578 | 3685 |
| HUMCACH1A_PEA_1_T1 (SEQ ID NO: 706) | 3155 | 3262 |
| HUMCACH1A_PEA_1_T2 (SEQ ID NO: 707) | 3578 | 3685 |
| HUMCACH1A_PEA_1_T3 (SEQ ID NO: 708) | 3578 | 3685 |
| HUMCACH1A_PEA_1_T4 (SEQ ID NO: 709) | 3578 | 3685 |
| HUMCACH1A_PEA_1_T6 (SEQ ID NO: 710) | 3578 | 3685 |
| HUMCACH1A_PEA_1_T7 (SEQ ID NO: 711) | 3578 | 3685 |
| HUMCACH1A_PEA_1_T8 (SEQ ID NO: 712) | 3518 | 3625 |
| HUMCACH1A_PEA_1_T12 (SEQ ID NO: 713) | 1911 | 2018 |
| HUMCACH1A_PEA_1_T13 (SEQ ID NO: 714) | 3578 | 3685 |
| HUMCACH1A_PEA_1_T14 (SEQ ID NO: 715) | 3578 | 3685 |
| HUMCACH1A_PEA_1_T15 (SEQ ID NO: 716) | 3578 | 3685 |

Segment cluster HUMCACH1A_PEA_{—}1_node_{—}68 (SEQ ID NO:773) according to the present invention is supported by 3 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T15 (SEQ ID NO:716). Table 92 below describes the starting and ending position of this segment on each transcript.

TABLE 92
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 3686 | 3738 |
| HUMCACH1A_PEA_1_T1 (SEQ ID NO: 706) | 3263 | 3315 |
| HUMCACH1A_PEA_1_T2 (SEQ ID NO: 707) | 3686 | 3738 |
| HUMCACH1A_PEA_1_T3 (SEQ ID NO: 708) | 3686 | 3738 |
| HUMCACH1A_PEA_1_T4 (SEQ ID NO: 709) | 3686 | 3738 |
| HUMCACH1A_PEA_1_T6 (SEQ ID NO: 710) | 3686 | 3738 |
| HUMCACH1A_PEA_1_T7 (SEQ ID NO: 711) | 3686 | 3738 |
| HUMCACH1A_PEA_1_T8 (SEQ ID NO: 712) | 3626 | 3678 |
| HUMCACH1A_PEA_1_T12 (SEQ ID NO: 713) | 2019 | 2071 |
| HUMCACH1A_PEA_1_T13 (SEQ ID NO: 714) | 3686 | 3738 |
| HUMCACH1A_PEA_1_T14 (SEQ ID NO: 715) | 3686 | 3738 |
| HUMCACH1A_PEA_1_T15 (SEQ ID NO: 716) | 3686 | 3738 |

Segment cluster HUMCACH1A_PEA_{—}1_node_{—}76 (SEQ ID NO:774) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T15 (SEQ ID NO:716). Table 93 below describes the starting and ending position of this segment on each transcript.

TABLE 93
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 4247 | 4357 |
| HUMCACH1A_PEA_1_T1 (SEQ ID NO: 706) | 3824 | 3934 |
| HUMCACH1A_PEA_1_T2 (SEQ ID NO: 707) | 4247 | 4357 |
| HUMCACH1A_PEA_1_T3 (SEQ ID NO: 708) | 4247 | 4357 |
| HUMCACH1A_PEA_1_T4 (SEQ ID NO: 709) | 4247 | 4357 |
| HUMCACH1A_PEA_1_T6 (SEQ ID NO: 710) | 4247 | 4357 |
| HUMCACH1A_PEA_1_T7 (SEQ ID NO: 711) | 4247 | 4357 |
| HUMCACH1A_PEA_1_T8 (SEQ ID NO: 712) | 4187 | 4297 |
| HUMCACH1A_PEA_1_T12 (SEQ ID NO: 713) | 2580 | 2690 |
| HUMCACH1A_PEA_1_T13 (SEQ ID NO: 714) | 4247 | 4357 |
| HUMCACH1A_PEA_1_T14 (SEQ ID NO: 715) | 4247 | 4357 |
| HUMCACH1A_PEA_1_T15 (SEQ ID NO: 716) | 4247 | 4357 |

Segment cluster HUMCACH1A_PEA_{—}1_node_{—}77 (SEQ ID NO:775) according to the present invention is supported by 1 libraries. The number of libraries was determined as previously described. This segment can be found in the following transcript(s): HUMCACH1A_PEA_{—}1_T15 (SEQ ID NO:716). Table 94 below describes the starting and ending position of this segment on each transcript.

TABLE 94
Segment location on transcripts

| | | Seg- |
|---|---|---|
| | Segment | ment |
| | starting | ending |
| Transcript name | position | position |
| HUMCACH1A_PEA_1_T15 (SEQ ID NO: 716) | 4358 | 4400 |

Segment cluster HUMCACH1A_PEA_{—}1_node_{—}79 (SEQ ID NO:776) according to the present invention is supported by 1 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714) and HUMCACH1A_PEA_{—}1_T14 (SEQ ID NO:715). Table 95 below describes the starting and ending position of this segment on each transcript.

TABLE 95
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 4358 | 4441 |
| HUMCACH1A_PEA_1_T1 (SEQ ID NO: 706) | 3935 | 4018 |
| HUMCACH1A_PEA_1_T2 (SEQ ID NO: 707) | 4358 | 4441 |
| HUMCACH1A_PEA_1_T3 (SEQ ID NO: 708) | 4358 | 4441 |
| HUMCACH1A_PEA_1_T4 (SEQ ID NO: 709) | 4358 | 4441 |
| HUMCACH1A_PEA_1_T6 (SEQ ID NO: 710) | 4358 | 4441 |
| HUMCACH1A_PEA_1_T7 (SEQ ID NO: 711) | 4358 | 4441 |
| HUMCACH1A_PEA_1_T8 (SEQ ID NO: 712) | 4298 | 4381 |
| HUMCACH1A_PEA_1_T12 (SEQ ID NO: 713) | 2691 | 2774 |
| HUMCACH1A_PEA_1_T13 (SEQ ID NO: 714) | 4358 | 4441 |
| HUMCACH1A_PEA_1_T14 (SEQ ID NO: 715) | 4358 | 4441 |

Segment cluster HUMCACH1A_PEA_{—}1_node_{—}81 (SEQ ID NO:777) according to the present invention is supported by 1 libraries. The number of libraries was determined as previously described. This segment can be found in the following transcript(s): HUMCACH1A_PEA_{—}1_T17 (SEQ ID NO:718). Table 96 below describes the starting and ending position of this segment on each transcript.

TABLE 96
Segment location on transcripts

| | | Seg- |
|---|---|---|
| | Segment | ment |
| | starting | ending |
| Transcript name | position | position |
| HUMCACH1A_PEA_1_T17 (SEQ ID NO: 718) | 1 | 31 |

Segment cluster HUMCACH1A_PEA_{—}1_node_{—}84 (SEQ ID NO:778) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T17 (SEQ ID NO:718). Table 97 below describes the starting and ending position of this segment on each transcript.

TABLE 97
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 4442 | 4486 |
| HUMCACH1A_PEA_1_T1 (SEQ ID NO: 706) | 4019 | 4063 |
| HUMCACH1A_PEA_1_T2 (SEQ ID NO: 707) | 4442 | 4486 |
| HUMCACH1A_PEA_1_T3 (SEQ ID NO: 708) | 4442 | 4486 |
| HUMCACH1A_PEA_1_T4 (SEQ ID NO: 709) | 4442 | 4486 |
| HUMCACH1A_PEA_1_T6 (SEQ ID NO: 710) | 4442 | 4486 |
| HUMCACH1A_PEA_1_T7 (SEQ ID NO: 711) | 4442 | 4486 |
| HUMCACH1A_PEA_1_T8 (SEQ ID NO: 712) | 4382 | 4426 |
| HUMCACH1A_PEA_1_T12 (SEQ ID NO: 713) | 2775 | 2819 |
| HUMCACH1A_PEA_1_T13 (SEQ ID NO: 714) | 4442 | 4486 |
| HUMCACH1A_PEA_1_T14 (SEQ ID NO: 715) | 4442 | 4486 |
| HUMCACH1A_PEA_1_T17 (SEQ ID NO: 718) | 32 | 76 |

Segment cluster HUMCACH1A_PEA_{—}1_node_{—}88 (SEQ ID NO:779) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T17 (SEQ ID NO:718). Table 98 below describes the starting and ending position of this segment on each transcript.

TABLE 98
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 4616 | 4681 |
| HUMCACH1A_PEA_1_T1 (SEQ ID NO: 706) | 4193 | 4258 |
| HUMCACH1A_PEA_1_T2 (SEQ ID NO: 707) | 4616 | 4681 |
| HUMCACH1A_PEA_1_T3 (SEQ ID NO: 708) | 4616 | 4681 |
| HUMCACH1A_PEA_1_T4 (SEQ ID NO: 709) | 4616 | 4681 |
| HUMCACH1A_PEA_1_T6 (SEQ ID NO: 710) | 4616 | 4681 |
| HUMCACH1A_PEA_1_T7 (SEQ ID NO: 711) | 4616 | 4681 |
| HUMCACH1A_PEA_1_T8 (SEQ ID NO: 712) | 4556 | 4621 |
| HUMCACH1A_PEA_1_T12 (SEQ ID NO: 713) | 2949 | 3014 |
| HUMCACH1A_PEA_1_T13 (SEQ ID NO: 714) | 4616 | 4681 |
| HUMCACH1A_PEA_1_T14 (SEQ ID NO: 715) | 4616 | 4681 |
| HUMCACH1A_PEA_1_T17 (SEQ ID NO: 718) | 206 | 271 |

Segment cluster HUMCACH1A_PEA_{—}1_node_{—}90 (SEQ ID NO:780) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T17 (SEQ ID NO:718). Table 99 below describes the starting and ending position of this segment on each transcript.

TABLE 99
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 4682 | 4773 |
| HUMCACH1A_PEA_1_T1 (SEQ ID NO: 706) | 4259 | 4350 |
| HUMCACH1A_PEA_1_T2 (SEQ ID NO: 707) | 4682 | 4773 |
| HUMCACH1A_PEA_1_T3 (SEQ ID NO: 708) | 4682 | 4773 |
| HUMCACH1A_PEA_1_T4 (SEQ ID NO: 709) | 4682 | 4773 |
| HUMCACH1A_PEA_1_T6 (SEQ ID NO: 710) | 4682 | 4773 |
| HUMCACH1A_PEA_1_T7 (SEQ ID NO: 711) | 4682 | 4773 |
| HUMCACH1A_PEA_1_T8 (SEQ ID NO: 712) | 4622 | 4713 |
| HUMCACH1A_PEA_1_T12 (SEQ ID NO: 713) | 3015 | 3106 |
| HUMCACH1A_PEA_1_T13 (SEQ ID NO: 714) | 4682 | 4773 |
| HUMCACH1A_PEA_1_T14 (SEQ ID NO: 715) | 4682 | 4773 |
| HUMCACH1A_PEA_1_T17 (SEQ ID NO: 718) | 272 | 363 |

Segment cluster HUMCACH1A_PEA_{—}1_node_{—}96 (SEQ ID NO:781) according to the present invention is supported by 4 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T17 (SEQ ID NO:718). Table 100 below describes the starting and ending position of this segment on each transcript.

TABLE 100
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 5062 | 5158 |
| HUMCACH1A_PEA_1_T1 (SEQ ID NO: 706) | 4639 | 4735 |
| HUMCACH1A_PEA_1_T2 (SEQ ID NO: 707) | 5062 | 5158 |
| HUMCACH1A_PEA_1_T3 (SEQ ID NO: 708) | 5062 | 5158 |
| HUMCACH1A_PEA_1_T4 (SEQ ID NO: 709) | 5062 | 5158 |
| HUMCACH1A_PEA_1_T6 (SEQ ID NO: 710) | 5062 | 5158 |
| HUMCACH1A_PEA_1_T7 (SEQ ID NO: 711) | 5062 | 5158 |
| HUMCACH1A_PEA_1_T8 (SEQ ID NO: 712) | 5002 | 5098 |
| HUMCACH1A_PEA_1_T12 (SEQ ID NO: 713) | 3395 | 3491 |
| HUMCACH1A_PEA_1_T13 (SEQ ID NO: 714) | 5062 | 5158 |
| HUMCACH1A_PEA_1_T14 (SEQ ID NO: 715) | 5062 | 5158 |
| HUMCACH1A_PEA_1_T17 (SEQ ID NO: 718) | 652 | 748 |

Segment cluster HUMCACH1A_PEA_{—}1_node_{—}98 (SEQ ID NO:782) according to the present invention is supported by 2 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T17 (SEQ ID NO:718). Table 101 below describes the starting and ending position of this segment on each transcript.

TABLE 101
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 5159 | 5261 |
| HUMCACH1A_PEA_1_T1 (SEQ ID NO: 706) | 4736 | 4838 |
| HUMCACH1A_PEA_1_T2 (SEQ ID NO: 707) | 5159 | 5261 |
| HUMCACH1A_PEA_1_T3 (SEQ ID NO: 708) | 5159 | 5261 |
| HUMCACH1A_PEA_1_T4 (SEQ ID NO: 709) | 5159 | 5261 |
| HUMCACH1A_PEA_1_T6 (SEQ ID NO: 710) | 5159 | 5261 |
| HUMCACH1A_PEA_1_T7 (SEQ ID NO: 711) | 5159 | 5261 |
| HUMCACH1A_PEA_1_T8 (SEQ ID NO: 712) | 5099 | 5201 |
| HUMCACH1A_PEA_1_T12 (SEQ ID NO: 713) | 3492 | 3594 |
| HUMCACH1A_PEA_1_T13 (SEQ ID NO: 714) | 5159 | 5261 |
| HUMCACH1A_PEA_1_T14 (SEQ ID NO: 715) | 5159 | 5261 |
| HUMCACH1A_PEA_1_T17 (SEQ ID NO: 718) | 749 | 851 |

Segment cluster HUMCACH1A_PEA_{—}1_node_{—}100 (SEQ ID NO:783) according to the present invention is supported by 1 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T14 (SEQ ID NO:715) and HUMCACH1A_PEA_{—}1_T17 (SEQ ID NO:718). Table 102 below describes the starting and ending position of this segment on each transcript.

TABLE 102
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 5262 | 5363 |
| HUMCACH1A_PEA_1_T1 (SEQ ID NO: 706) | 4839 | 4940 |
| HUMCACH1A_PEA_1_T2 (SEQ ID NO: 707) | 5262 | 5363 |
| HUMCACH1A_PEA_1_T3 (SEQ ID NO: 708) | 5262 | 5363 |
| HUMCACH1A_PEA_1_T4 (SEQ ID NO: 709) | 5262 | 5363 |
| HUMCACH1A_PEA_1_T6 (SEQ ID NO: 710) | 5262 | 5363 |
| HUMCACH1A_PEA_1_T7 (SEQ ID NO: 711) | 5262 | 5363 |
| HUMCACH1A_PEA_1_T8 (SEQ ID NO: 712) | 5202 | 5303 |
| HUMCACH1A_PEA_1_T12 (SEQ ID NO: 713) | 3595 | 3696 |
| HUMCACH1A_PEA_1_T13 (SEQ ID NO: 714) | 5262 | 5363 |
| HUMCACH1A_PEA_1_T14 (SEQ ID NO: 715) | 5262 | 5363 |
| HUMCACH1A_PEA_1_T17 (SEQ ID NO: 718) | 852 | 953 |

Segment cluster HUMCACH1A_PEA_{—}1_node_{—}101 (SEQ ID NO:784) according to the present invention is supported by 0 libraries. The number of libraries was determined as previously described. This segment can be found in the following transcript(s): HUMCACH1A_PEA_{—}1_T14 (SEQ ID NO:715). Table 103 below describes the starting and ending position of this segment on each transcript.

TABLE 103
Segment location on transcripts

| | | Seg- |
|---|---|---|
| | Segment | ment |
| | starting | ending |
| Transcript name | position | position |
| HUMCACH1A_PEA_1_T14 (SEQ ID NO: 715) | 5364 | 5440 |

Segment cluster HUMCACH1A_PEA_{—}1_node_{—}107 (SEQ ID NO:785) according to the present invention is supported by 6 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T13 (SEQ ID NO:714), HUMCACH1A_PEA_{—}1_T17 (SEQ ID NO:718), HUMCACH1A_PEA_{—}1_T18 (SEQ ID NO:719) and HUMCACH1A_PEA_{—}1_T19 (SEQ ID NO:720). Table 104 below describes the starting and ending position of this segment on each transcript.

TABLE 104
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 5495 | 5611 |
| HUMCACH1A_PEA_1_T1 (SEQ ID NO: 706) | 5072 | 5188 |
| HUMCACH1A_PEA_1_T2 (SEQ ID NO: 707) | 5495 | 5611 |
| HUMCACH1A_PEA_1_T3 (SEQ ID NO: 708) | 5495 | 5611 |
| HUMCACH1A_PEA_1_T4 (SEQ ID NO: 709) | 5495 | 5611 |
| HUMCACH1A_PEA_1_T6 (SEQ ID NO: 710) | 5495 | 5611 |
| HUMCACH1A_PEA_1_T7 (SEQ ID NO: 711) | 5495 | 5611 |
| HUMCACH1A_PEA_1_T8 (SEQ ID NO: 712) | 5435 | 5551 |
| HUMCACH1A_PEA_1_T12 (SEQ ID NO: 713) | 3828 | 3944 |
| HUMCACH1A_PEA_1_T13 (SEQ ID NO: 714) | 5495 | 5611 |
| HUMCACH1A_PEA_1_T17 (SEQ ID NO: 718) | 1085 | 1201 |
| HUMCACH1A_PEA_1_T18 (SEQ ID NO: 719) | 336 | 452 |
| HUMCACH1A_PEA_1_T19 (SEQ ID NO: 720) | 1457 | 1573 |

Segment cluster HUMCACH1A_PEA_{—}1_node_{—}111 (SEQ ID NO:786) according to the present invention is supported by 0 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T8 (SEQ ID NO:712), HUMCACH1A_PEA_{—}1_T12 (SEQ ID NO:713), HUMCACH1A_PEA_{—}1_T17 (SEQ ID NO:718), HUMCACH1A_PEA_{—}1_T18 (SEQ ID NO:719) and HUMCACH1A_PEA_{—}1_T19 (SEQ ID NO:720). Table 105 below describes the starting and ending position of this segment on each transcript.

TABLE 105
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 5980 | 6006 |
| HUMCACH1A_PEA_1_T1 (SEQ ID NO: 706) | 5557 | 5583 |
| HUMCACH1A_PEA_1_T2 (SEQ ID NO: 707) | 5980 | 6006 |
| HUMCACH1A_PEA_1_T3 (SEQ ID NO: 708) | 5980 | 6006 |
| HUMCACH1A_PEA_1_T4 (SEQ ID NO: 709) | 5980 | 6006 |
| HUMCACH1A_PEA_1_T6 (SEQ ID NO: 710) | 5980 | 6006 |
| HUMCACH1A_PEA_1_T8 (SEQ ID NO: 712) | 5920 | 5946 |
| HUMCACH1A_PEA_1_T12 (SEQ ID NO: 713) | 4313 | 4339 |
| HUMCACH1A_PEA_1_T17 (SEQ ID NO: 718) | 1570 | 1596 |
| HUMCACH1A_PEA_1_T18 (SEQ ID NO: 719) | 821 | 847 |
| HUMCACH1A_PEA_1_T19 (SEQ ID NO: 720) | 1942 | 1968 |

Segment cluster HUMCACH1A_PEA_{—}1_node_{—}117 (SEQ ID NO:787) according to the present invention is supported by 1 libraries. The number of libraries was determined as previously described. This segment can be found in the following transcript(s): HUMCACH1A_PEA_{—}1_T13 (SEQ ID NO:714). Table 106 below describes the starting and ending position of this segment on each transcript.

TABLE 106
Segment location on transcripts

| | | Seg- |
|---|---|---|
| | Segment | ment |
| | starting | ending |
| Transcript name | position | position |
| HUMCACH1A_PEA_1_T13 (SEQ ID NO: 714) | 6294 | 6365 |

Segment cluster HUMCACH1A_PEA_{—}1_node_{—}124 (SEQ ID NO:788) according to the present invention is supported by 18 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T2 (SEQ ID NO:707), HUMCACH1A_PEA_{—}1_T3 (SEQ ID NO:708), HUMCACH1A_PEA_{—}1_T4 (SEQ ID NO:709), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T17 (SEQ ID NO:718), HUMCACH1A_PEA_{—}1_T18 (SEQ ID NO:719) and HUMCACH1A_PEA_{—}1_T19 (SEQ ID NO:720). Table 107 below describes the starting and ending position of this segment on each transcript.

TABLE 107
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 7551 | 7649 |
| HUMCACH1A_PEA_1_T1 (SEQ ID NO: 706) | 7128 | 7226 |
| HUMCACH1A_PEA_1_T2 (SEQ ID NO: 707) | 7551 | 7649 |
| HUMCACH1A_PEA_1_T3 (SEQ ID NO: 708) | 7551 | 7649 |
| HUMCACH1A_PEA_1_T4 (SEQ ID NO: 709) | 7551 | 7649 |
| HUMCACH1A_PEA_1_T6 (SEQ ID NO: 710) | 7730 | 7828 |
| HUMCACH1A_PEA_1_T7 (SEQ ID NO: 711) | 7524 | 7622 |
| HUMCACH1A_PEA_1_T8 (SEQ ID NO: 712) | 7491 | 7589 |
| HUMCACH1A_PEA_1_T12 (SEQ ID NO: 713) | 5884 | 5982 |
| HUMCACH1A_PEA_1_T17 (SEQ ID NO: 718) | 3141 | 3239 |
| HUMCACH1A_PEA_1_T18 (SEQ ID NO: 719) | 2392 | 2490 |
| HUMCACH1A_PEA_1_T19 (SEQ ID NO: 720) | 3513 | 3611 |

Segment cluster HUMCACH1A_PEA_{—}1_node_{—}126 (SEQ ID NO:789) according to the present invention is supported by 9 libraries. The number of libraries was determined as previously described. This segment can be found in the following transcript(s): HUMCACH1A_PEA_{—}1_T0 (SEQ ID NO:705), HUMCACH1A_PEA_{—}1_T1 (SEQ ID NO:706), HUMCACH1A_PEA_{—}1_T6 (SEQ ID NO:710), HUMCACH1A_PEA_{—}1_T7 (SEQ ID NO:711), HUMCACH1A_PEA_{—}1_T8 (SEQ ID NO:712), HUMCACH1A_PEA_{—}1_T12 (SEQ ID NO:713), HUMCACH1A_PEA_{—}1_T17 (SEQ ID NO:718), HUMCACH1A_PEA_{—}1_T18 (SEQ ID NO:719) and HUMCACH1A_PEA_{—}1_T19 (SEQ ID NO:720). Table 108 below describes the starting and ending position of this segment on each transcript.

TABLE 108
Segment location on transcripts

| | | Seg- |
|---|---|---|
| | Segment | ment |
| | starting | ending |
| Transcript name | position | position |
| HUMCACH1A_PEA_1_T0 (SEQ ID NO: 705) | 9311 | 9380 |
| HUMCACH1A_PEA_1_T1 (SEQ ID NO: 706) | 8888 | 8957 |
| HUMCACH1A_PEA_1_T6 (SEQ ID NO: 710) | 9490 | 9559 |
| HUMCACH1A_PEA_1_T7 (SEQ ID NO: 711) | 9284 | 9353 |
| HUMCACH1A_PEA_1_T8 (SEQ ID NO: 712) | 9251 | 9320 |
| HUMCACH1A_PEA_1_T12 (SEQ ID NO: 713) | 7644 | 7713 |
| HUMCACH1A_PEA_1_T17 (SEQ ID NO: 718) | 4901 | 4970 |
| HUMCACH1A_PEA_1_T18 (SEQ ID NO: 719) | 4152 | 4221 |
| HUMCACH1A_PEA_1_T19 (SEQ ID NO: 720) | 5273 | 5342 |

### Variant Protein Alignment to the Previously Known Protein:

Sequence name: CCAD_HUMAN_V3 (SEQ ID NO:791)
Sequence documentation:
Alignment of: HUMCACH1A_PEA_1_P7 (SEQ ID NO:796) × CCAD_HUMAN_V3 (SEQ ID
NO:791) ..
Alignment segment 1/1:

| | | | |
|---|---|---|---|
| Quality: | 16625.00 | Escore: | 0 |
| Matching length: | 1696 | Total length: | 1716 |
| Matching Percent Similarity: | 99.94 | Matching Percent Identity: | 99.94 |
| Total Percent Similarity: | 98.78 | Total Percent Identity: | 98.78 |
| Gaps: | 1 | | |
| Alignment: | | | |

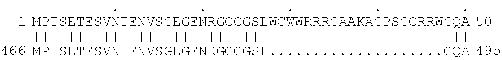

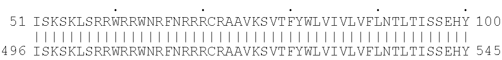

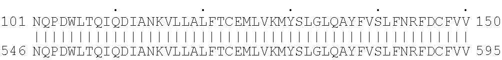

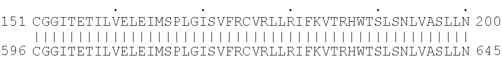

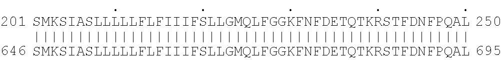

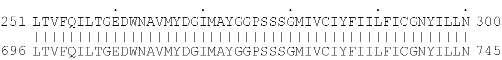

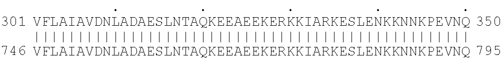

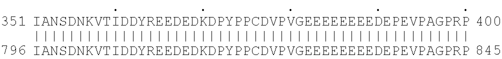

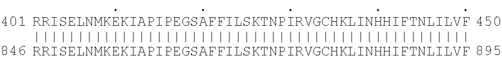

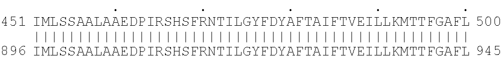

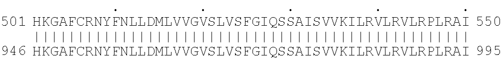

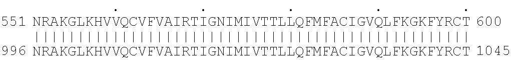

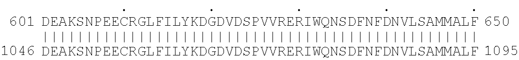

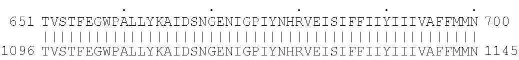

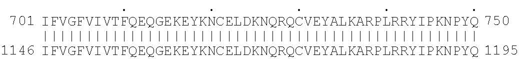

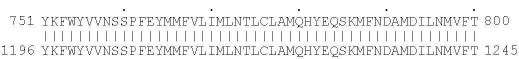

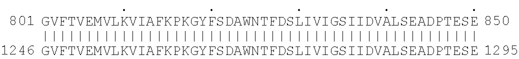

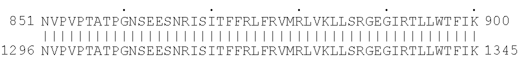

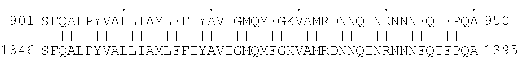

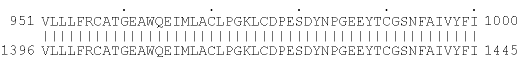

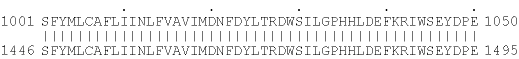

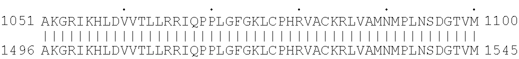

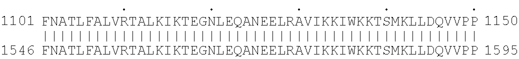

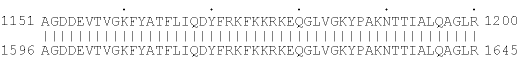

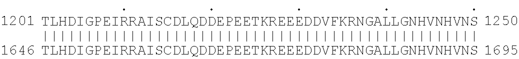

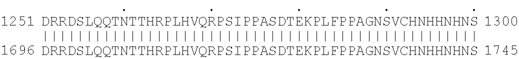

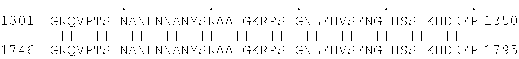

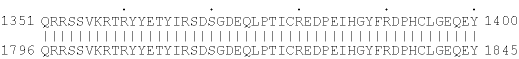

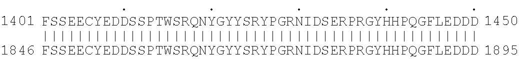

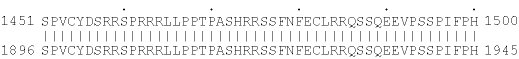

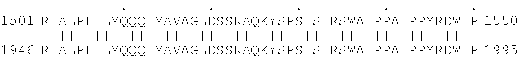

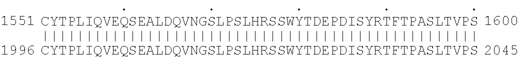

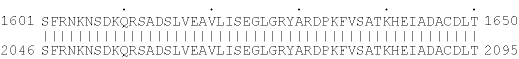

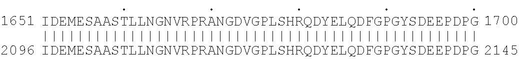

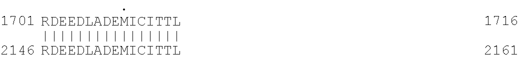

Sequence name: CCAD_HUMAN (SEQ ID NO:790)
Sequence documentation:
Alignment of: HUMCACH1A_PEA_1_P13 (SEQ ID NO:802) × OCAD_HUMAN (SEQ ID
NO:790) ..
Alignment segment 1/1:

| | | | |
|---|---|---|---|
| Quality: | 5658.00 | Escore: | 0 |
| Matching length: | 564 | Total length: | 564 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

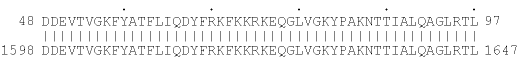

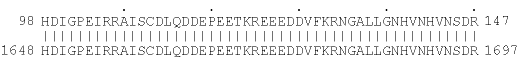

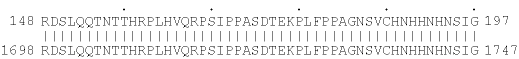

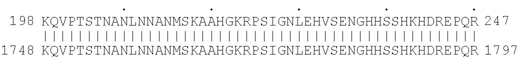

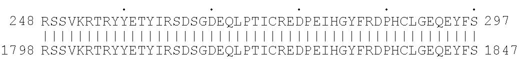

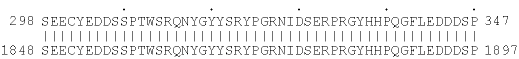

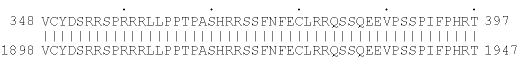

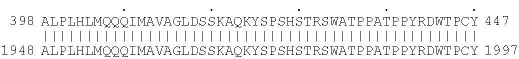

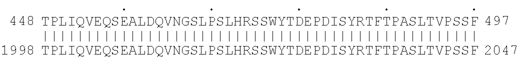

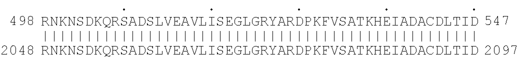

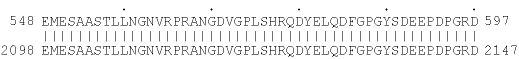

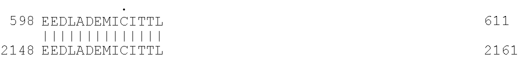

Sequence name: CCAD_HUMAN (SEQ ID NO:790)
Sequence documentation:
Alignment of: HUMCACH1A_PEA_1_{— P14 (SEQ ID NO:803) × CCAD}_HUMAN (SEQ ID
NO:790) ..
Alignment segment 1/1:

| | | | |
|---|---|---|---|
| Quality: | 4021.00 | Escore: | 0 |
| Matching length: | 399 | Total length: | 399 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

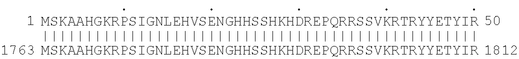

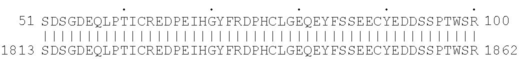

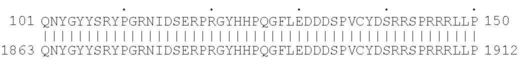

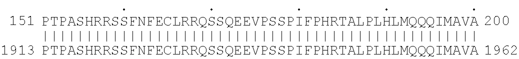

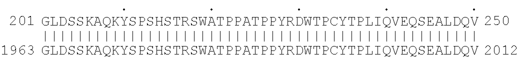

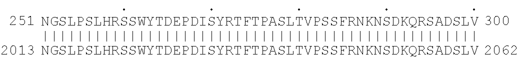

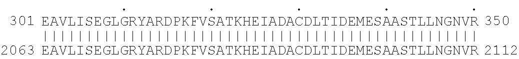

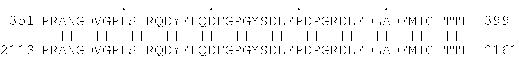

Sequence name: CCAD_HUMAN (SEQ ID NO:790)
Sequence documentation:
Alignment of: HUMCACH1A_PEA_1_P17 (SEQ ID NO:805) × CCAD_HUMAN (SEQ ID
NO:790) ..
Alignment segment 1/1:

| | | | |
|---|---|---|---|
| Quality: | 3976.00 | Escore: | 0 |
| Matching length: | 407 | Total length: | 407 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

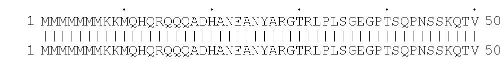

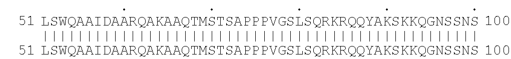

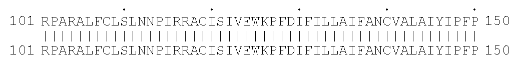

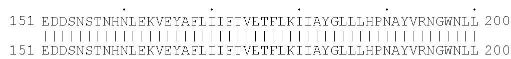

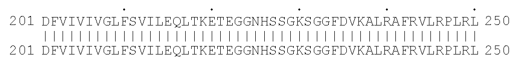

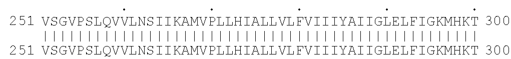

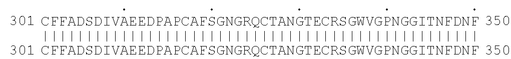

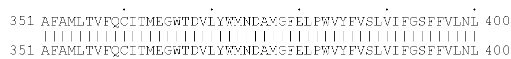

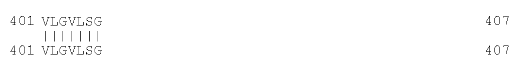

Expression of Voltage-Dependent L-Type Calcium Channel Alpha-1D Subunit Calcium Channel, L Type, Alpha-1 Polypeptide, Isoform 2 Transcripts which are Detectable by Seg 113, 35, 109, 125, in Normal, and Cancerous Colon Tissues

Expression of Voltage-dependent L-type calcium channel alpha-1D subunit Calcium channel, L type, alpha-1 polypeptide, isoform 2 transcripts detectable by or according to segments 113, 35, 109, 125 was measured with oligonucleotide-based micro-arrays. The results of image intensities for each feature were normalized according to the ninetieth percentile of the image intensities of all the features on the chip. Then, feature image intensities for replicates of the same oligonucleotide on the chip and replicates of the same sample were averaged. Outlying results were discarded.

For every oligonucleotide HUMCACH1A_{—}0_{—}3_{—}14917, HUMCACH1A_{—}0_{—}0_{—}14922, HUMCACH1A_{—}0_{—}0_{—}14924 and HUMCACH1A_{—}0_{—}0_{—}14913 (SEQ ID NOs: 1331, 1332, 1333 and 1334, respectively) the averaged intensity determined for every sample was divided by the averaged intensity of all the normal samples (Sample Nos. 62-66 and 69, Table 1, above, “Tissue samples in testing panel”), to obtain a value of fold up-regulation for each sample relative to the averaged normal samples. These data are presented in a histogram bellow (FIG. 47). As is evident from the histogram, the expression of Voltage-dependent L-type calcium channel alpha-1D subunit Calcium channel, L type, alpha-1 polypeptide, isoform 2 transcripts detectable with the above oligonucleotides in cancer samples was higher than in the normal samples.

HUMCACH1A_0_3_14917-

(SEQ ID NO: 1331)

AGAGAATATCACTCCGATGGTCGGTTTCTGACTGTCACGCTAAGGGCAAC
HUMCACH1A_0_0_14922-

(SEQ ID NO: 1332)

GAACACAGAGAACGTCAGCGGTGAAGGCGAGAACCGAGGCTGCTGTGGAA
HUMCACH1A_0_0_14924-

(SEQ ID NO: 1333)

GGCCCAGCATTGGGAACCTTGAGCATGTGTCTGAAAATGGGCATCATTCT
HUMCACH1A_0_0_14913-

(SEQ ID NO: 1334)

GACTCAGGAGATGAACAGCTCCCAACTATTTGCCGGGAAGACCCAGAGAT

Expression of Voltage-Dependent L-Type Calcium Channel Alpha-1D Subunit Calcium Channel, L Type, Alpha-1 Polypeptide, Isoform 2

HUMCACH1A Transcripts which are Detectable by Amplicon as Depicted in Sequence Name HUMCACH1Aseg101 (SEQ ID NO: 1337) in Normal and Cancerous Colon Tissues

Expression of Voltage-dependent L-type calcium channel alpha-1D subunit Calcium channel, L type, alpha-1 polypeptide, isoform 2 transcripts detectable by or according to seg101, HUMCACH1A seg101 amplicon (SEQ ID NO: 1337) and HUMCACH1Aseg101F (SEQ ID NO: 1335), HUMCACH1A seg101R (SEQ ID NO: 1336) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

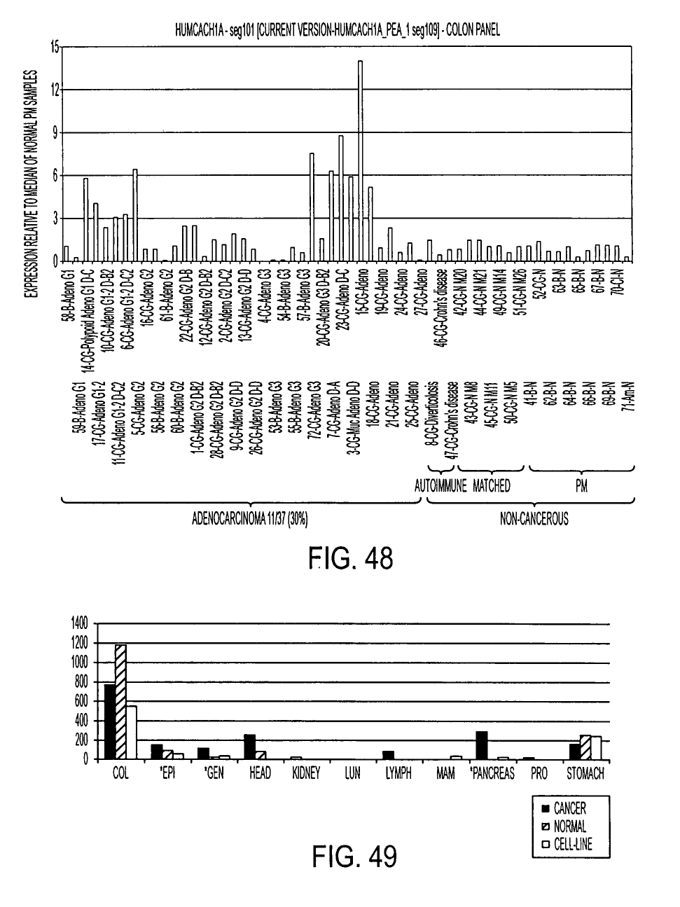

FIG. 48 is a histogram showing over expression of the above-indicated Voltage-dependent L-type calcium channel alpha-1D subunit Calcium channel, L type, alpha-1 polypeptide, isoform 2 transcripts in cancerous colon samples relative to the normal samples. The number and percentage of samples that exhibit at least 3 fold over-expression, out of the total number of samples tested is indicated in the bottom.

As is evident from FIG. 48, the expression of Voltage-dependent L-type calcium channel alpha-1D subunit Calcium channel, L type, alpha-1 polypeptide, isoform 2 transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 3 fold was found in II out of 37 adenocarcinoma samples,

Statistical analysis was applied to verify the significance of these results, as described below.

The P value for the difference in the expression levels of Voltage-dependent L-type calcium channel alpha-1D subunit Calcium channel, L type, alpha-1 polypeptide, isoform 2 transcripts detectable by the above amplicon in colon cancer samples versus the normal tissue samples was determined by T test as 1.02E-03.

Threshold of 3 fold overexpression was found to differentiate between cancer and normal samples with P value of 3.78E-02 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: HUMCACH1Aseg101F forward primer (SEQ ID NO: 1335); and HUMCACH1A seg101R reverse primer (SEQ ID NO: 1336).

The present invention also preferably encompasses any amplicon obtained through the use of any suitable primer pair; for example, for the above experiment, the following amplicon was obtained as a non-limiting illustrative example only of a suitable amplicon: HUMCACH1A seg101 (SEQ ID NO: 1337).

Forward primer (SEQ ID NO: 1335):
CAGCAGGAAATTCGGTGTGTC
Reverse primer (SEQ ID NO: 1336):
TCAAGGTTCCCAATGCTGG
Amplicon (SEQ ID NO: 1337):
CAGCAGGAAATTCGGTGTGTCATAACCATCATAACCATAATTCCATAGGA
AAGCAAGTTCCCACCTCAACAAATGCCAATCTCAATAATGCCAATATGTC
CAAAGCTGCCCATGGAAAGCGGCCCAGCATTGGGAACCTTGA

### Description for Cluster HUMCEA

Cluster HUMCEA features 10 transcript(s) and 47 segment(s) of interest, the names for which are given in Tables 1 and 2, respectively, the sequences themselves are given at the end of the application. The selected protein variants are given in table 3.

TABLE 1
Transcripts of interest

| Transcripts Name | SEQ ID NO: |
|---|---|
| HUMCEA_PEA_1_T8 | 806 |
| HUMCEA_PEA_1_T9 | 807 |
| HUMCEA_PEA_1_T12 | 808 |
| HUMCEA_PEA_1_T14 | 809 |
| HUMCEA_PEA_1_T16 | 810 |
| HUMCEA_PEA_1_T20 | 811 |
| HUMCEA_PEA_1_T25 | 812 |
| HUMCEA_PEA_1_T26 | 813 |
| HUMCEA_PEA_1_T29 | 814 |
| HUMCEA_PEA_1_T30 | 815 |

TABLE 2
Segments of interest

| Segment Name | SEQ ID NO: |
|---|---|
| HUMCEA_PEA_1_node_0 | 816 |
| HUMCEA_PEA_1_node_2 | 817 |
| HUMCEA_PEA_1_node_6 | 818 |
| HUMCEA_PEA_1_node_11 | 819 |
| HUMCEA_PEA_1_node_12 | 820 |
| HUMCEA_PEA_1_node_31 | 821 |
| HUMCEA_PEA_1_node_36 | 822 |
| HUMCEA_PEA_1_node_42 | 823 |
| HUMCEA_PEA_1_node_43 | 824 |
| HUMCEA_PEA_1_node_44 | 825 |
| HUMCEA_PEA_1_node_46 | 826 |
| HUMCEA_PEA_1_node_48 | 827 |
| HUMCEA_PEA_1_node_63 | 828 |
| HUMCEA_PEA_1_node_65 | 829 |
| HUMCEA_PEA_1_node_67 | 830 |
| HUMCEA_PEA_1_node_3 | 831 |
| HUMCEA_PEA_1_node_7 | 832 |
| HUMCEA_PEA_1_node_8 | 833 |
| HUMCEA_PEA_1_node_9 | 834 |
| HUMCEA_PEA_1_node_10 | 835 |
| HUMCEA_PEA_1_node_15 | 836 |
| HUMCEA_PEA_1_node_16 | 837 |
| HUMCEA_PEA_1_node_17 | 838 |
| HUMCEA_PEA_1_node_18 | 839 |
| HUMCEA_PEA_1_node_19 | 840 |
| HUMCEA_PEA_1_node_20 | 841 |
| HUMCEA_PEA_1_node_21 | 842 |
| HUMCEA_PEA_1_node_22 | 843 |
| HUMCEA_PEA_1_node_23 | 844 |
| HUMCEA_PEA_1_node_24 | 845 |
| HUMCEA_PEA_1_node_27 | 846 |
| HUMCEA_PEA_1_node_29 | 847 |
| HUMCEA_PEA_1_node_30 | 848 |
| HUMCEA_PEA_1_node_33 | 849 |
| HUMCEA_PEA_1_node_34 | 850 |
| HUMCEA_PEA_1_node_35 | 851 |
| HUMCEA_PEA_1_node_45 | 852 |
| HUMCEA_PEA_1_node_49 | 853 |
| HUMCEA_PEA_1_node_50 | 854 |
| HUMCEA_PEA_1_node_51 | 855 |
| HUMCEA_PEA_1_node_56 | 856 |
| HUMCEA_PEA_1_node_57 | 857 |
| HUMCEA_PEA_1_node_58 | 858 |
| HUMCEA_PEA_1_node_60 | 859 |
| HUMCEA_PEA_1_node_61 | 860 |
| HUMCEA_PEA_1_node_62 | 861 |
| HUMCEA_PEA_1_node_64 | 862 |

TABLE 3
Proteins of interest

| Protein Name | SEQ ID NO: | Corresponding Transcript(s) |
|---|---|---|
| HUMCEA_PEA_1_P4 | 864 | HUMCEA_PEA_1_T8 |
| | | (SEQ ID NO: 806) |
| HUMCEA_PEA_1_P5 | 865 | HUMCEA_PEA_1_T9 |
| | | (SEQ ID NO: 807) |
| HUMCEA_PEA_1_P7 | 866 | HUMCEA_PEA_1_T12 |
| | | (SEQ ID NO: 808) |
| HUMCEA_PEA_1_P10 | 867 | HUMCEA_PEA_1_T16 |
| | | (SEQ ID NO: 810) |
| HUMCEA_PEA_1_P14 | 868 | HUMCEA_PEA_1_T20 |
| | | (SEQ ID NO: 811) |
| HUMCEA_PEA_1_P19 | 869 | HUMCEA_PEA_1_T25 |
| | | (SEQ ID NO: 812) |
| HUMCEA_PEA_1_P20 | 870 | HUMCEA_PEA_1_T26 |
| | | (SEQ ID NO: 813) |

These sequences are variants of the known protein Carcinoembryonic antigen-related cell adhesion molecule 5 precursor (SwissProt accession identifier CEA5_HUMAN; known also according to the synonyms Carcinoembryonic antigen; CEA; Meconium antigen 100; CD66e antigen), SEQ ID NO: 863, referred to herein as the previously known protein.

The sequence for protein Carcinoembryonic antigen-related cell adhesion molecule 5 precursor (SEQ ID NO:863) is given at the end of the application, as “Carcinoembryonic antigen-related cell adhesion molecule 5 precursor amino acid sequence”. Known polymorphisms for this sequence are as shown in Table 4.

TABLE 4
Amino acid mutations for Known Protein

| SNP position(s) on amino | |
|---|---|
| acid sequence | Comment |
| 320 | Missing |

Protein Carcinoembryonic antigen-related cell adhesion molecule 5 precursor (SEQ ID NO:863) localization is believed to be Attached to the membrane by a GPI-anchor.

The previously known protein also has the following indication(s) and/or potential therapeutic use(s): Cancer. It has been investigated for clinical/therapeutic use in humans, for example as a target for an antibody or small molecule, and/or as a direct therapeutic; available information related to these investigations is as follows. Potential pharmaceutically related or therapeutically related activity or activities of the previously known protein are as follows: Immunostimulant. A therapeutic role for a protein represented by the cluster has been predicted. The cluster was assigned this field because there was information in the drug database or the public databases (e.g., described herein above) that this protein, or part thereof, is used or can be used for a potential therapeutic indication: Imaging agent; Anticancer; Immunostimulant; Immunoconjugate; Monoclonal antibody, murine; Antisense therapy; antibody.

The following GO Annotation(s) apply to the previously known protein. The following annotation(s) were found: integral plasma membrane protein; membrane, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nim.nih.gov/projects/LocusLink/>.

Cluster HUMCEA can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 49 and Table 5. This cluster is overexpressed (at least at a minimum level) in the following pathological conditions: epithelial malignant tumors, a mixture of malignant tumors from different tissues and pancreas carcinoma.

TABLE 5
Normal tissue distribution

| Name of Tissue | Number |
|---|---|
| colon | 1175 |
| epithelial | 92 |
| general | 29 |
| head and neck | 81 |
| kidney | 0 |
| lung | 0 |
| lymph nodes | 0 |
| breast | 0 |
| pancreas | 0 |
| prostate | 0 |
| stomach | 256 |

TABLE 6
P values and ratios for expression in cancerous tissue

| Name of Tissue | P1 | P2 | SP1 | R3 | SP2 | R4 |
|---|---|---|---|---|---|---|
| colon | 2.0e−01 | 2.7e−01 | 9.8e−01 | 0.5 | 1 | 0.5 |
| epithelial | 2.1e−03 | 2.7e−02 | 6.4e−04 | 1.4 | 2.1e−01 | 1.0 |
| general | 3.9e−08 | 8.2e−06 | 9.2e−18 | 3.2 | 1.3e−10 | 2.2 |
| head and neck | 3.4e−01 | 5.0e−01 | 2.1e−01 | 1.8 | 5.6e−01 | 0.9 |
| kidney | 4.3e−01 | 5.3e−01 | 5.8e−01 | 2.1 | 7.0e−01 | 1.6 |
| lung | 1.3e−01 | 2.6e−01 | 1 | 1.1 | 1 | 1.1 |
| lymph nodes | 3.1e−01 | 5.7e−01 | 8.1e−02 | 6.0 | 3.3e−01 | 2.5 |
| breast | 3.8e−01 | 1.5e−01 | 1 | 1.0 | 6.8e−01 | 1.5 |
| pancreas | 2.2e−02 | 2.3e−02 | 1.4e−08 | 7.8 | 7.4e−07 | 6.4 |
| prostate | 5.3e−01 | 6.0e−01 | 3.0e−01 | 2.5 | 4.2e−01 | 2.0 |
| stomach | 1.5e−01 | 4.7e−01 | 8.9e−01 | 0.6 | 7.2e−01 | 0.4 |

For this cluster, at least one oligonucleotide was found to demonstrate overexpression of the cluster, although not of at least one transcript/segment as listed below. Microarray (chip) data is also available for this cluster as follows. Various oligonucleotides were tested for being differentially expressed in various disease conditions, particularly cancer, as previously described. The following oligonucleotides were found to hit this cluster but not other segments/transcripts below, shown in Table 7.

TABLE 7
Oligonucleotides related to this cluster

| Oligonucleotide name | Overexpressed in cancers | Chip reference |
|---|---|---|
| HUMCEA_0_0_15168 | lung malignant tumors | LUN |

As noted above, cluster HUMCEA features 10 transcript(s), which were listed in Table 1 above. These transcript(s) encode for protein(s) which are variant(s) of protein Carcinoembryonic antigen-related cell adhesion molecule 5 precursor (SEQ ID NO:863). A description of each variant protein according to the present invention is now provided.

Variant protein HUMCEA_PEA_{—}1_P4 (SEQ ID NO:864) according to the present invention has an amino acid sequence as given at the end of the application; it is encoded by transcript(s) HUMCEA_PEA_{—}1_T8 (SEQ ID NO:806). An alignment is given to the known protein (Carcinoembryonic antigen-related cell adhesion molecule 5 precursor (SEQ ID NO:863)) at the end of the application. One or more alignments to one or more previously published protein sequences are given at the end of the application. A brief description of the relationship of the variant protein according to the present invention to each such aligned protein is as follows:

Comparison Report Between HUMCEA_PEA_{—}1_P4 (SEQ ID NO:864) and CEA5_HUMAN (SEQ ID NO:863):

1. An isolated chimeric polypeptide encoding for HUMCEA_PEA_{—}1_P4 (SEQ ID NO:864), comprising a first amino acid sequence being at least 90% homologous to MESPSAPPHRWCIPWQRLLLTASLLTFWNPPTTAKLTIESTPFNVAEGKEVLLLVHNLPQHLFGYSWYKGE RVDGNRQIIGYVIGTQQATPGPAYSGREIIYPNASLLIQNIIQNDTGFYTLHVIKSDLVNEEATGQFRVYPEL PKPSISSNNSKPVEDKDAVAFTCEPETQDATYLWWVNNQSLPVSPRLQLSNGNRTLTLFNVTRNDTASYK CETQNPVSARRSDSVILNVL corresponding to amino acids 1-234 of CEA5_HUMAN (SEQ ID NO:863), which also corresponds to amino acids 1-234 of HUMCEA_PEA_{—}1_P4 (SEQ ID NO:864), and a second amino acid sequence being at least 70%, optionally at least 80%, preferably at least 85%, more preferably at least 90% and most preferably at least 95% homologous to a polypeptide having the sequence CEYICSSLAQAASPNPQGQRQDFSVPLRFKYTDPQPWTSRLSVTFCPRKTWADQVLTKNRRGGAASVLGG SGSTPYDGRNR (SEQ ID NO:1475) corresponding to amino acids 235-315 of HUMCEA_PEA_{—}1_P4 (SEQ ID NO:864), wherein said first amino acid sequence and second amino acid sequence are contiguous and in a sequential order.

2. An isolated polypeptide encoding for a tail of HUMCEA_PEA_{—}1_P4 (SEQ ID NO:864), comprising a polypeptide being at least 70%, optionally at least about 80%, preferably at least about 85%, more preferably at least about 90% and most preferably at least about 95% homologous to the sequence CEYICSSLAQAASPNPQGQRQDFSVPLRFKYTDPQPWTSRLSVTFCPRKTWADQVLTKNRRGGAASVLGG SGSTPYDGRNR (SEQ ID NO:1475) in HUMCEA_PEA_{—}1_P4 (SEQ ID NO:864).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMCEA_PEA_{—}1_P4 (SEQ ID NO:864) also has the following non-silent SNPs (Single Nucleotide Polymorphisms) as listed in Table 8, (given according to their position(s) on the amino acid sequence, with the alternative amino acid(s) listed; the last column indicates whether the SNP is known or not; the presence of known SNPs in variant protein HUMCEA_PEA_{—}1_P4 (SEQ ID NO:864) sequence provides support for the deduced sequence of this variant protein according to the present invention).

TABLE 8
Amino acid mutations

| SNP position(s) on | Alternative | Previously |
|---|---|---|
| amino acid sequence | amino acid(s) | known SNP? |
| 63 | F -> L | No |
| 80 | I -> V | Yes |
| 83 | V -> A | Yes |
| 137 | Q -> P | Yes |
| 173 | D -> N | No |

The glycosylation sites of variant protein HUMCEA_PEA_{—}1_P4 (SEQ ID NO:864), as compared to the known protein Carcinoembryonic antigen-related cell adhesion molecule 5 precursor (SEQ ID NO:863), are described in Table 9 (given according to their position(s) on the amino acid sequence in the first column; the second column indicates whether the glycosylation site is present in the variant protein; and the last column indicates whether the position is different on the variant protein).

TABLE 9
Glycosylation site(s)

| Position(s) on | | |
|---|---|---|
| known amino acid | Present in | Position in |
| sequence | variant protein? | variant protein? |
| 197 | yes | 197 |
| 466 | no | |
| 360 | no | |
| 288 | no | |
| 665 | no | |
| 560 | no | |
| 650 | no | |
| 480 | no | |
| 104 | yes | 104 |
| 580 | no | |
| 204 | yes | 204 |
| 115 | yes | 115 |
| 208 | yes | 208 |
| 152 | yes | 152 |
| 309 | no | |
| 432 | no | |
| 351 | no | |
| 246 | no | |
| 182 | yes | 182 |
| 612 | no | |
| 256 | no | |
| 508 | no | |
| 330 | no | |
| 274 | no | |
| 292 | no | |
| 553 | no | |
| 529 | no | |
| 375 | no | |

Variant protein HUMCEA_PEA_{—}1_P4 (SEQ ID NO:864) is encoded by the following transcript(s): HUMCEA_PEA_{—}1_T8 (SEQ ID NO:806), for which the sequence(s) is/are given at the end of the application. The coding portion of transcript HUMCEA_PEA_{—}1_T8 (SEQ ID NO:806) is shown in bold; this coding portion starts at position 115 and ends at position 1059. The transcript also has the following SNPs as listed in Table 10 (given according to their position on the nucleotide sequence, with the alternative nucleic acid listed; the last column indicates whether the SNP is known or not; the presence of known SNPs in variant protein HUMCEA_PEA_{—}1_P4 (SEQ ID NO:864) sequence provides support for the deduced sequence of this variant protein according to the present invention).

TABLE 10
Nucleic acid SNPs

| SNP position | | |
|---|---|---|
| on nucleotide | Alternative | Previously |
| sequence | nucleic acid | known SNP? |
| 49 | T -> | No |
| 273 | A -> C | Yes |
| 303 | T -> G | No |
| 324 | T -> C | Yes |
| 352 | A -> G | Yes |
| 362 | T -> C | Yes |
| 524 | A -> C | Yes |
| 631 | G -> A | No |
| 1315 | A -> G | No |
| 1380 | T -> C | No |
| 1533 | C -> A | Yes |
| 1706 | G -> A | Yes |
| 2308 | T -> C | No |
| 2362 | C -> T | No |
| 2455 | A -> | No |
| 2504 | C -> A | Yes |
| 2558 | G -> | No |
| 2623 | G -> | No |
| 2639 | T -> A | No |
| 2640 | T -> A | No |
| 2832 | G -> A | Yes |
| 2885 | C -> T | No |
| 3396 | A -> G | Yes |
| 3562 | C -> T | Yes |
| 3753 | C -> T | Yes |

Variant protein HUMCEA_PEA_{—}1_P5 (SEQ ID NO:865) according to the present invention has an amino acid sequence as given at the end of the application; it is encoded by transcript(s) HUMCEA_PEA_{—}1_T9 (SEQ ID NO:807). An alignment is given to the known protein (Carcinoembryonic antigen-related cell adhesion molecule 5 precursor (SEQ ID NO:863)) at the end of the application. One or more alignments to one or more previously published protein sequences are given at the end of the application. A brief description of the relationship of the variant protein according to the present invention to each such aligned protein is as follows:

Comparison Report Between HUMCEA_PEA_{—}1_P5 (SEQ ID NO:865) and CEA5_HUMAN (SEQ ID NO:863):

1. An isolated chimeric polypeptide encoding for HUMCEA_PEA_{—}1_P5 (SEQ ID NO:865), comprising a first amino acid sequence being at least 90% homologous to MESPSAPPHRWCIPWQRLLLTASLLTFWNPPTTAKLTIESTPFNVAEGKEVLLLVHNLPQHLFGYSWYKGE RVDGNRQIIGYVIGTQQATPGPAYSGREIIYPNASLLIQNIIQNDTGFYTLHVIKSDLVNEEATGQFRVYPEL PKPSISSNNSKPVEDKDAVAFTCEPETQDATYLWWVNNQSLPVSPRLQLSNGNRTLTLFNVTRNDTASYK CETQNPVSARRSDSVILNVLYGPDAPTISPLNTSYRSGENLNLSCHAASNPPAQYSWFVNGTFQQSTQELFI PNITVNNSGSYTCQAHNSDTGLNRTTVTTITVYAEPPKPFITSNNSNPVEDEDAVALTCEPEIQNTTYLWW VNNQSLPVSPRLQLSNDNRTLTLLSVTRNDVGPYECGIQNELSVDHSDPVILNVLYGPDDPTISPSYTYYRP GVNLSLSCHAASNPPAQYSWLIDGNIQQHTQELFISNITEKNSGLYTCQANNSASGHSRTTVKTITVSAELP KPSISSNNSKPVEDKDAVAFTCEPEAQNTTYLWWVNGQSLPVSPRLQLSNGNRTLTLFNVTRNDARAYVC GIQNSVSANRSDPVTLDVLYGPDTPIISPPDSSYLSGANLNLSCHSASNPSPQYSWRINGIPQQHTQVLFIAKI TPNNNGTYACFVSNLATGRNNSIVKSITVS corresponding to amino acids 1-675 of CEA5_HUMAN (SEQ ID NO:863), which also corresponds to amino acids 1-675 of HUMCEA_PEA_{—}1_P5 (SEQ ID NO:865), and a second amino acid sequence being at least 70%, optionally at least 80%, preferably at least 85%, more preferably at least 90% and most preferably at least 95% homologous to a polypeptide having the sequence GKWLPGASASYSGVESIWFSPKSQEDIFFPSLCSMGTRKSQILS (SEQ ID NO:1476) corresponding to amino acids 676-719 of HUMCEA_PEA_{—}1_P5 (SEQ ID NO:865), wherein said first amino acid sequence and second amino acid sequence are contiguous and in a sequential order.

2. An isolated polypeptide encoding for a tail of HUMCEA_PEA_{—}1_P5 (SEQ ID NO:865), comprising a polypeptide being at least 70%, optionally at least about 80%, preferably at least about 85%, more preferably at least about 90% and most preferably at least about 95% homologous to the sequence GKWLPGASASYSGVESIWFSPKSQEDIFFPSLCSMGTRKSQILS (SEQ ID NO:1476) in HUMCEA_PEA_{—}1_P5 (SEQ ID NO:865).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMCEA_PEA_{—}1_P5 (SEQ ID NO:865) also has the following non-silent SNPs (Single Nucleotide Polymorphisms) as listed in Table 11, (given according to their position(s) on the amino acid sequence, with the alternative amino acid(s) listed; the last column indicates whether the SNP is known or not; the presence of known SNPs in variant protein HUMCEA_PEA_{—}1_P5 (SEQ ID NO:865) sequence provides support for the deduced sequence of this variant protein according to the present invention).

TABLE 11
Amino acid mutations

| SNP position(s) | | |
|---|---|---|
| on amino acid | Alternative | Previously |
| sequence | amino acid(s) | known SNP? |
| 63 | F -> L | No |
| 80 | I -> V | Yes |
| 83 | V -> A | Yes |
| 137 | Q -> P | Yes |
| 173 | D -> N | No |
| 289 | I -> T | No |
| 340 | A -> D | Yes |
| 398 | E -> K | Yes |
| 647 | P -> | No |
| 664 | R -> S | Yes |

The glycosylation sites of variant protein HUMCEA_PEA_{—}1_P5 (SEQ ID NO:865), as compared to the known protein Carcinoembryonic antigen-related cell adhesion molecule 5 precursor (SEQ ID NO:863), are described in Table 12 (given according to their position(s) on the amino acid sequence in the first column; the second column indicates whether the glycosylation site is present in the variant protein; and the last column indicates whether the position is different on the variant protein).

TABLE 12
Glycosylation site(s)

| Position(s) on | | |
|---|---|---|
| known amino acid | Present in | Position in |
| sequence | variant protein? | variant protein? |
| 197 | yes | 197 |
| 466 | yes | 466 |
| 360 | yes | 360 |
| 288 | yes | 288 |
| 665 | yes | 665 |
| 560 | yes | 560 |
| 650 | yes | 650 |
| 480 | yes | 480 |
| 104 | yes | 104 |
| 580 | yes | 580 |
| 204 | yes | 204 |
| 115 | yes | 115 |
| 208 | yes | 208 |
| 152 | yes | 152 |
| 309 | yes | 309 |
| 432 | yes | 432 |
| 351 | yes | 351 |
| 246 | yes | 246 |
| 182 | yes | 182 |
| 612 | yes | 612 |
| 256 | yes | 256 |
| 508 | yes | 508 |
| 330 | yes | 330 |
| 274 | yes | 274 |
| 292 | yes | 292 |
| 553 | yes | 553 |
| 529 | yes | 529 |
| 375 | yes | 375 |

Variant protein HUMCEA_PEA_{—}1_P5 (SEQ ID NO:865) is encoded by the following transcript(s): HUMCEA_PEA_{—}1_T9 (SEQ ID NO:807), for which the sequence(s) is/are given at the end of the application. The coding portion of transcript HUMCEA_PEA_{—}1_T9 (SEQ ID NO:807) is shown in bold; this coding portion starts at position 115 and ends at position 2271. The transcript also has the following SNPs as listed in Table 13 (given according to their position on the nucleotide sequence, with the alternative nucleic acid listed; the last column indicates whether the SNP is known or not; the presence of known SNPs in variant protein HUMCEA_PEA_{—}1_P5 (SEQ ID NO:865) sequence provides support for the deduced sequence of this variant protein according to the present invention).

TABLE 13
Nucleic acid SNPs

| SNP position | | |
|---|---|---|
| on nucleotide | Alternative | Previously |
| sequence | nucleic acid | known SNP? |
| 49 | T -> | No |
| 273 | A -> C | Yes |
| 303 | T -> G | No |
| 324 | T -> C | Yes |
| 352 | A -> G | Yes |
| 362 | T -> C | Yes |
| 524 | A -> C | Yes |
| 631 | G -> A | No |
| 915 | A -> G | No |
| 980 | T -> C | No |
| 1133 | C -> A | Yes |
| 1306 | G -> A | Yes |
| 1908 | T -> C | No |
| 1962 | C -> T | No |
| 2055 | A -> | No |
| 2104 | C -> A | Yes |
| 3259 | T -> C | Yes |

Variant protein HUMCEA_PEA_{—}1_P7 (SEQ ID NO:866) according to the present invention has an amino acid sequence as given at the end of the application; it is encoded by transcript(s) HUMCEA_PEA_{—}1_T12 (SEQ ID NO:808). An alignment is given to the known protein (Carcinoembryonic antigen-related cell adhesion molecule 5 precursor (SEQ ID NO:863)) at the end of the application. One or more alignments to one or more previously published protein sequences are given at the end of the application. A brief description of the relationship of the variant protein according to the present invention to each such aligned protein is as follows:

Comparison Report Between HUMCEA_PEA_{—}1_P7 (SEQ ID NO:866) and CEA5_HUMAN (SEQ ID NO:863):

1. An isolated chimeric polypeptide encoding for HUMCEA_PEA_{—}1_P7 (SEQ ID NO:866), comprising a first amino acid sequence being at least 90% homologous to MESPSAPPHRWCIPWQRLLLTASLLTFWNPPTTAKLTIESTPFNVAEGKEVLLLVHNLPQHLFGYSWYKGE RVDGNRQIIGYVIGTQQATPGPAYSGREIIYPNASLLIQNIIQNDTGFYTLHVIKSDLVNEEATGQFRVYPEL PKPSISSNNSKPVEDKDAVAFTCEPETQDATYLWWVNNQSLPVSPRLQLSNGNRTLTLFNVTRNDTASYK CETQNPVSARRSDSVILNVLYGPDAPTISPLNTSYRSGENLNLSCHAASNPPAQYSWFVNGTFQQSTQELFI PNITVNNSGSYTCQAHNSDTGLNRTTVTTITVYAEPPKPFITSNNSNPVEDEDAVALTCEPEIQNTTYLWW VNNQSLPVSPRLQLSNDNRTLTLLSVTRNDVGPYECGIQNELSVDHSDPVILNVLYGPDDPTISPSYTYYRP GVNLSLSCHAASNPPAQYSWLIDGNIQQHTQELFISNITEKNSGLYTCQANNSASGHSRTTVKTITVSAELP KPSISSNNSKPVEDKDAVAFTCEPEAQNTTYLWWVNGQSLPVSPRLQLSNGNRTLTLFNVTRNDARAYVC GIQNSVSANRSDPVTLDVLYGPDTPIISPPDSSYLSGANLNLSCHSASNPSPQYSWRINGIPQQHTQVLFIAKI TPNNNGTYACFVSNLATGRNNSIVKSITV corresponding to amino acids 1-674 of CEA5_HUMAN (SEQ ID NO:863), which also corresponds to amino acids 1-674 of HUMCEA_PEA_{—}1_P7 (SEQ ID NO:866), and a second amino acid sequence being at least 90% homologous to SAGATVGIMIGVLVGVALI corresponding to amino acids 684-702 of CEA5_HUMAN (SEQ ID NO:863), which also corresponds to amino acids 675-693 of HUMCEA_PEA_{—}1_P7 (SEQ ID NO:866), wherein said first amino acid sequence and second amino acid sequence are contiguous and in a sequential order.

2. An isolated chimeric polypeptide encoding for an edge portion of HUMCEA_PEA_{—}1_P7 (SEQ ID NO:866), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise VS, having a structure as follows: a sequence starting from any of amino acid numbers 674−x to 674; and ending at any of amino acid numbers 675+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: membrane. The protein localization is believed to be membrane because of manual inspection of known protein localization and/or gene structure.

Variant protein HUMCEA_PEA_{—}1_P7 (SEQ ID NO:866) also has the following non-silent SNPs (Single Nucleotide Polymorphisms) as listed in Table 14, (given according to their position(s) on the amino acid sequence, with the alternative amino acid(s) listed; the last column indicates whether the SNP is known or not; the presence of known SNPs in variant protein HUMCEA_PEA_{—}1_P7 (SEQ ID NO:866) sequence provides support for the deduced sequence of this variant protein according to the present invention).

TABLE 14
Amino acid mutations

| SNP position(s) | | |
|---|---|---|
| on amino acid | Alternative | Previously |
| sequence | amino acid(s) | known SNP? |
| 63 | F -> L | No |
| 80 | I -> V | Yes |
| 83 | V -> A | Yes |
| 137 | Q -> P | Yes |
| 173 | D -> N | No |
| 289 | I -> T | No |
| 340 | A -> D | Yes |
| 398 | E -> K | Yes |
| 647 | P -> | No |
| 664 | R -> S | Yes |

The glycosylation sites of variant protein HUMCEA_PEA_{—}1_P7 (SEQ ID NO:866), as compared to the known protein Carcinoembryonic antigen-related cell adhesion molecule 5 precursor (SEQ ID NO:863), are described in Table 15 (given according to their position(s) on the amino acid sequence in the first column; the second column indicates whether the glycosylation site is present in the variant protein; and the last column indicates whether the position is different on the variant protein).

TABLE 15
Glycosylation site(s)

| Position(s) on | | |
|---|---|---|
| known amino acid | Present in | Position in |
| sequence | variant protein? | variant protein? |
| 197 | yes | 197 |
| 466 | yes | 466 |
| 360 | yes | 360 |
| 288 | yes | 288 |
| 665 | yes | 665 |
| 560 | yes | 560 |
| 650 | yes | 650 |
| 480 | yes | 480 |
| 104 | yes | 104 |
| 580 | yes | 580 |
| 204 | yes | 204 |
| 115 | yes | 115 |
| 208 | yes | 208 |
| 152 | yes | 152 |
| 309 | yes | 309 |
| 432 | yes | 432 |
| 351 | yes | 351 |
| 246 | yes | 246 |
| 182 | yes | 182 |
| 612 | yes | 612 |
| 256 | yes | 256 |
| 508 | yes | 508 |
| 330 | yes | 330 |
| 274 | yes | 274 |
| 292 | yes | 292 |
| 553 | yes | 553 |
| 529 | yes | 529 |
| 375 | yes | 375 |

Variant protein HUMCEA_PEA_{—}1_P7 (SEQ ID NO:866) is encoded by the following transcript(s): HUMCEA_PEA_{—}1_T12 (SEQ ID NO:808), for which the sequence(s) is/are given at the end of the application. The coding portion of transcript HUMCEA_PEA_{—}1_T12 (SEQ ID NO:808) is shown in bold; this coding portion starts at position 115 and ends at position 2193. The transcript also has the following SNPs as listed in Table 16 (given according to their position on the nucleotide sequence, with the alternative nucleic acid listed; the last column indicates whether the SNP is known or not; the presence of known SNPs in variant protein HUMCEA_PEA_{—}1_P7 (SEQ ID NO:866) sequence provides support for the deduced sequence of this variant protein according to the present invention).

TABLE 16
Nucleic acid SNPs

| SNP position | | |
|---|---|---|
| on nucleotide | Alternative | Previously |
| sequence | nucleic acid | known SNP? |
| 49 | T -> | No |
| 273 | A -> C | Yes |
| 303 | T -> G | No |
| 324 | T -> C | Yes |
| 352 | A -> G | Yes |
| 362 | T -> C | Yes |
| 524 | A -> C | Yes |
| 631 | G -> A | No |
| 915 | A -> G | No |
| 980 | T -> C | No |
| 1133 | C -> A | Yes |
| 1306 | G -> A | Yes |
| 1908 | T -> C | No |
| 1962 | C -> T | No |
| 2055 | A -> | No |
| 2104 | C -> A | Yes |
| 2196 | G -> | No |
| 2212 | T -> A | No |
| 2213 | T -> A | No |
| 2405 | G -> A | Yes |
| 2458 | C -> T | No |
| 2969 | A -> G | Yes |
| 3135 | C -> T | Yes |
| 3326 | C -> T | Yes |

Variant protein HUMCEA_PEA_{—}1_P10 (SEQ ID NO:867) according to the present invention has an amino acid sequence as given at the end of the application; it is encoded by transcript(s) HUMCEA_PEA_{—}1_T16 (SEQ ID NO:810). An alignment is given to the known protein (Carcinoembryonic antigen-related cell adhesion molecule 5 precursor (SEQ ID NO:863)) at the end of the application. One or more alignments to one or more previously published protein sequences are given at the end of the application. A brief description of the relationship of the variant protein according to the present invention to each such aligned protein is as follows:

Comparison Report Between HUMCEA_PEA_{—}1_P10 (SEQ ID NO:867) and CEA5_HUMAN (SEQ ID NO:863):

1. An isolated chimeric polypeptide encoding for HUMCEA_PEA_{—}1_P10 (SEQ ID NO:867), comprising a first amino acid sequence being at least 90% homologous to MESPSAPPHRWCIPWQRLLLTASLLTFWNPPTTAKLTIESTPFNVAEGKEVLLLVHNLPQHLFGYSWYKGE RVDGNRQIIGYVIGTQQATPGPAYSGREIIYPNASLLIQNIIQNDTGFYTLHVIKSDLVNEEATGQFRVYPEL PKPSISSNNSKPVEDKDAVAFTCEPETQDATYLWWVNNQSLPVSPRLQLSNGNRTLTLFNVTRNDTASYK CETQNPVSARRSDS corresponding to amino acids 1-228 of CEA5_HUMAN (SEQ ID NO:863), which also corresponds to amino acids 1-228 of HUMCEA_PEA_{—}1_P10 (SEQ ID NO:867), and a second amino acid sequence being at least 90% homologous to VILNVLYGPDDPTISPSYTYYRPGVNLSLSCHAASNPPAQYSWLIDGNIQQHTQELFISNITEKNSGLYTCQA NNSASGHSRTTVKTITVSAELPKPSISSNNSKPVEDKDAVAFTCEPEAQNTTYLWWVNGQSLPVSPRLQLS NGNRTLTLFNVTRNDARAYVCGIQNSVSANRSDPVTLDVLYGPDTPIISPPDSSYLSGANLNLSCHSASNPS PQYSWRINGIPQQHTQVLFIAKITPNNNGTYACFVSNLATGRNNSIVKSITVSASGTSPGLSAGATVGIMIGV LVGVALI corresponding to amino acids 407-702 of CEA5_HUMAN (SEQ ID NO:863), which also corresponds to amino acids 229-524 of HUMCEA_PEA_{—}1_P10 (SEQ ID NO:867), wherein said first amino acid sequence and second amino acid sequence are contiguous and in a sequential order.

2. An isolated chimeric polypeptide encoding for an edge portion of HUMCEA_PEA_{—}1_P10 (SEQ ID NO:867), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise SV, having a structure as follows: a sequence starting from any of amino acid numbers 228−x to 228; and ending at any of amino acid numbers 229+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide.

Variant protein HUMCEA_PEA_{—}1_P10 (SEQ ID NO:867) also has the following non-silent SNPs (Single Nucleotide Polymorphisms) as listed in Table 17, (given according to their position(s) on the amino acid sequence, with the alternative amino acid(s) listed; the last column indicates whether the SNP is known or not; the presence of known SNPs in variant protein HUMCEA_PEA_{—}1_P10 (SEQ ID NO:867) sequence provides support for the deduced sequence of this variant protein according to the present invention).

TABLE 17
Amino acid mutations

| SNP position(s) | | |
|---|---|---|
| on amino acid | Alternative | Previously |
| sequence | amino acid(s) | known SNP? |
| 63 | F -> L | No |
| 80 | I -> V | Yes |
| 83 | V -> A | Yes |
| 137 | Q -> P | Yes |
| 173 | D -> N | No |
| 469 | P -> | No |
| 486 | R -> S | Yes |
| 504 | G -> | No |

The glycosylation sites of variant protein HUMCEA_PEA_{—}1_P10 (SEQ ID NO:867), as compared to the known protein Carcinoembryonic antigen-related cell adhesion molecule 5 precursor (SEQ ID NO:863), are described in Table 18 (given according to their position(s) on the amino acid sequence in the first column; the second column indicates whether the glycosylation site is present in the variant protein; and the last column indicates whether the position is different on the variant protein).

TABLE 18
Glycosylation site(s)

| Position(s) on | | |
|---|---|---|
| known amino acid | Present in | Position in |
| sequence | variant protein? | variant protein? |
| 197 | yes | 197 |
| 466 | yes | 288 |
| 360 | no | |
| 288 | no | |
| 665 | yes | 487 |
| 560 | yes | 382 |
| 650 | yes | 472 |
| 480 | yes | 302 |
| 104 | yes | 104 |
| 580 | yes | 402 |
| 204 | yes | 204 |
| 115 | yes | 115 |
| 208 | yes | 208 |
| 152 | yes | 152 |
| 309 | no | |
| 432 | yes | 254 |
| 351 | no | |
| 246 | no | |
| 182 | yes | 182 |
| 612 | yes | 434 |
| 256 | no | |
| 508 | yes | 330 |
| 330 | no | |
| 274 | no | |
| 292 | no | |
| 553 | yes | 375 |
| 529 | yes | 351 |
| 375 | no | |

Variant protein HUMCEA_PEA_{—}1_P10 (SEQ ID NO:867) is encoded by the following transcript(s): HUMCEA_PEA_{—}1_T16 (SEQ ID NO:810), for which the sequence(s) is/are given at the end of the application. The coding portion of transcript HUMCEA_PEA_{—}1_T16 (SEQ ID NO:810) is shown in bold; this coding portion starts at position 115 and ends at position 1686. The transcript also has the following SNPs as listed in Table 19 (given according to their position on the nucleotide sequence, with the alternative nucleic acid listed; the last column indicates whether the SNP is known or not; the presence of known SNPs in variant protein HUMCEA_PEA_{—}1_P10 (SEQ ID NO:867) sequence provides support for the deduced sequence of this variant protein according to the present invention).

TABLE 19
Nucleic acid SNPs

| SNP position | | |
|---|---|---|
| on nucleotide | Alternative | Previously |
| sequence | nucleic acid | known SNP? |
| 49 | T -> | No |
| 273 | A -> C | Yes |
| 303 | T -> G | No |
| 324 | T -> C | Yes |
| 352 | A -> G | Yes |
| 362 | T -> C | Yes |
| 524 | A -> C | Yes |
| 631 | G -> A | No |
| 1374 | T -> C | No |
| 1428 | C -> T | No |
| 1521 | A -> | No |
| 1570 | C -> A | Yes |
| 1624 | G -> | No |
| 1689 | G -> | No |
| 1705 | T -> A | No |
| 1706 | T -> A | No |
| 1898 | G -> A | Yes |
| 1951 | C -> T | No |
| 2462 | A -> G | Yes |
| 2628 | C -> T | Yes |
| 2819 | C -> T | Yes |

Variant protein HUMCEA_PEA_{—}1_P14 (SEQ ID NO:868) according to the present invention has an amino acid sequence as given at the end of the application; it is encoded by transcript(s) HUMCEA_PEA_{—}1_T20 (SEQ ID NO:811). The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein HUMCEA_PEA_{—}1_P14 (SEQ ID NO:868) also has the following non-silent SNPs (Single Nucleotide Polymorphisms) as listed in Table 20, (given according to their position(s) on the amino acid sequence, with the alternative amino acid(s) listed; the last column indicates whether the SNP is known or not; the presence of known SNPs in variant protein HUMCEA_PEA_{—}1_P14 (SEQ ID NO:868) sequence provides support for the deduced sequence of this variant protein according to the present invention).

TABLE 20
Amino acid mutations

| SNP position(s) | | |
|---|---|---|
| on amino acid | Alternative | Previously |
| sequence | amino acid(s) | known SNP? |
| 63 | F -> L | No |
| 80 | I -> V | Yes |
| 83 | V -> A | Yes |
| 137 | Q -> P | Yes |
| 173 | D -> N | No |
| 289 | I -> T | No |
| 340 | A -> D | Yes |
| 398 | E -> K | Yes |

Variant protein HUMCEA_PEA_{—}1_P14 (SEQ ID NO:868) is encoded by the following transcript(s): HUMCEA_PEA_{—}1_T20 (SEQ ID NO:811), for which the sequence(s) is/are given at the end of the application. The coding portion of transcript HUMCEA_PEA_{—}1_T20 (SEQ ID NO:811) is shown in bold; this coding portion starts at position 115 and ends at position 1821. The transcript also has the following SNPs as listed in Table 21 (given according to their position on the nucleotide sequence, with the alternative nucleic acid listed; the last column indicates whether the SNP is known or not; the presence of known SNPs in variant protein HUMCEA_PEA_{—}1_P14 (SEQ ID NO:868) sequence provides support for the deduced sequence of this variant protein according to the present invention).

TABLE 21
Nucleic acid SNPs

| SNP position | | |
|---|---|---|
| on nucleotide | Alternative | Previously |
| sequence | nucleic acid | known SNP? |
| 49 | T -> | No |
| 273 | A -> C | Yes |
| 303 | T -> G | No |
| 324 | T -> C | Yes |
| 352 | A -> G | Yes |
| 362 | T -> C | Yes |
| 524 | A -> C | Yes |
| 631 | G -> A | No |
| 915 | A -> G | No |
| 980 | T -> C | No |
| 1133 | C -> A | Yes |
| 1306 | G -> A | Yes |

Variant protein HUMCEA_PEA_{—}1_P19 (SEQ ID NO:869) according to the present invention has an amino acid sequence as given at the end of the application; it is encoded by transcript(s) HUMCEA_PEA_{—}1_T25 (SEQ ID NO:812). An alignment is given to the known protein (Carcinoembryonic antigen-related cell adhesion molecule 5 precursor (SEQ ID NO:863)) at the end of the application. One or more alignments to one or more previously published protein sequences are given at the end of the application. A brief description of the relationship of the variant protein according to the present invention to each such aligned protein is as follows:

Comparison Report Between HUMCEA_PEA_{—}1_P19 (SEQ ID NO:869) and CEA5_HUMAN (SEQ ID NO:863):

1. An isolated chimeric polypeptide encoding for HUMCEA_PEA_{—}1_P19 (SEQ ID NO:869), comprising a first amino acid sequence being at least 90% homologous to MESPSAPPHRWCIPWQRLLLTASLLTFWNPPTTAKLTIESTPFNVAEGKEVLLLVHNLPQHLFGYSWYKGE RVDGNRQIIGYVIGTQQATPGPAYSGREIIYPNASLLIQNIIQNDTGFYTLHVIKSDLVNEEATGQFRVYPEL PKPSISSNNSKPVEDKDAVAFTCEPETQDATYLWWVNNQSLPVSPRLQLSNGNRTLTLFNVTRNDTASYK CETQNPVSARRSDSVILN corresponding to amino acids 1-232 of CEA5_HUMAN (SEQ ID NO:863), which also corresponds to amino acids 1-232 of HUMCEA_PEA_{—}1_P19 (SEQ ID NO:869), and a second amino acid sequence being at least 90% homologous to VLYGPDTPIISPPDSSYLSGANLNLSCHSASNPSPQYSWRINGIPQQHTQVLFIAKITPNNNGTYACFVSNLA TGRNNSIVKSITVSASGTSPGLSAGATVGIMIGVLVGVALI corresponding to amino acids 589-702 of CEA5_HUMAN (SEQ ID NO:863), which also corresponds to amino acids 233-346 of HUMCEA_PEA_{—}1_P19 (SEQ ID NO:869), wherein said first amino acid sequence and second amino acid sequence are contiguous and in a sequential order.

2. An isolated chimeric polypeptide encoding for an edge portion of HUMCEA_PEA_{—}1_P19 (SEQ ID NO:869), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise NV, having a structure as follows: a sequence starting from any of amino acid numbers 232−x to 232; and ending at any of amino acid numbers 233+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: membrane. The protein localization is believed to be membrane because of manual inspection of known protein localization and/or gene structure.

Variant protein HUMCEA_PEA_{—}1_P19 (SEQ ID NO:869) also has the following non-silent SNPs (Single Nucleotide Polymorphisms) as listed in Table 22, (given according to their position(s) on the amino acid sequence, with the alternative amino acid(s) listed; the last column indicates whether the SNP is known or not; the presence of known SNPs in variant protein HUMCEA_PEA_{—}1_P19 (SEQ ID NO:869) sequence provides support for the deduced sequence of this variant protein according to the present invention).

TABLE 22
Amino acid mutations

| SNP position(s) | | |
|---|---|---|
| on amino acid | Alternative | Previously |
| sequence | amino acid(s) | known SNP? |
| 63 | F -> L | No |
| 80 | I -> V | Yes |
| 83 | V -> A | Yes |
| 137 | Q -> P | Yes |
| 173 | D -> N | No |
| 291 | P -> | No |
| 308 | R -> S | Yes |
| 326 | G -> | No |

The glycosylation sites of variant protein HUMCEA_PEA_{—}1_P19 (SEQ ID NO:869), as compared to the known protein Carcinoembryonic antigen-related cell adhesion molecule 5 precursor (SEQ ID NO:863), are described in Table 23 (given according to their position(s) on the amino acid sequence in the first column; the second column indicates whether the glycosylation site is present in the variant protein; and the last column indicates whether the position is different on the variant protein).

TABLE 23
Glycosylation site(s)

| Position(s) on | | |
|---|---|---|
| known amino acid | Present in | Position in |
| sequence | variant protein? | variant protein? |
| 197 | yes | 197 |
| 466 | no | |
| 360 | no | |
| 288 | no | |
| 665 | yes | 309 |
| 560 | no | |
| 650 | yes | 294 |
| 480 | no | |
| 104 | yes | 104 |
| 580 | no | |
| 204 | yes | 204 |
| 115 | yes | 115 |
| 208 | yes | 208 |
| 152 | yes | 152 |
| 309 | no | |
| 432 | no | |
| 351 | no | |
| 246 | no | |
| 182 | yes | 182 |
| 612 | yes | 256 |
| 256 | no | |
| 508 | no | |
| 330 | no | |
| 274 | no | |
| 292 | no | |
| 553 | no | |
| 529 | no | |
| 375 | no | |

Variant protein HUMCEA_PEA_{—}1_P19 (SEQ ID NO:869) is encoded by the following transcript(s): HUMCEA_PEA_{—}1_T25 (SEQ ID NO:812), for which the sequence(s) is/are given at the end of the application. The coding portion of transcript HUMCEA_PEA_{—}1_T25 (SEQ ID NO:812) is shown in bold; this coding portion starts at position 115 and ends at position 1152. The transcript also has the following SNPs as listed in Table 24 (given according to their position on the nucleotide sequence, with the alternative nucleic acid listed; the last column indicates whether the SNP is known or not; the presence of known SNPs in variant protein HUMCEA_PEA_{—}1_P19 (SEQ ID NO:869) sequence provides support for the deduced sequence of this variant protein according to the present invention).

TABLE 24
Nucleic acid SNPs

| SNP position on | | |
|---|---|---|
| nucleotide sequence | Alternative nucleic acid | Previously known SNP? |
| 49 | T -> | No |
| 273 | A -> C | Yes |
| 303 | T -> G | No |
| 324 | T -> C | Yes |
| 352 | A -> G | Yes |
| 362 | T -> C | Yes |
| 524 | A -> C | Yes |
| 631 | G -> A | No |
| 840 | T -> C | No |
| 894 | C -> T | No |
| 987 | A -> | No |
| 1036 | C -> A | Yes |
| 1090 | G -> | No |
| 1155 | G -> | No |
| 1171 | T -> A | No |
| 1172 | T -> A | No |
| 1364 | G -> A | Yes |
| 1417 | C -> T | No |
| 1928 | A -> G | Yes |
| 2094 | C -> T | Yes |
| 2285 | C -> T | Yes |

Variant protein HUMCEA_PEA_{—}1_P20 (SEQ ID NO:870) according to the present invention has an amino acid sequence as given at the end of the application; it is encoded by transcript(s) HUMCEA_PEA_{—}1_T26 (SEQ ID NO:813). An alignment is given to the known protein (Carcinoembryonic antigen-related cell adhesion molecule 5 precursor (SEQ ID NO:863)) at the end of the application. One or more alignments to one or more previously published protein sequences are given at the end of the application. A brief description of the relationship of the variant protein according to the present invention to each such aligned protein is as follows:

Comparison Report Between HUMCEA_PEA_{—}1_P20 (SEQ ID NO:870) and CEA5_HUMAN (SEQ ID NO:863):

1. An isolated chimeric polypeptide encoding for HUMCEA_PEA_{—}1_P20 (SEQ ID NO:870), comprising a first amino acid sequence being at least 90% homologous to MESPSAPPHRWCIPWQRLLLTASLLTFWNPPTTAKLTIESTPFNVAEGKEVLLLVHNLPQHLFGYSWYKGE RVDGNRQIIGYVIGTQQATPGPAYSGREIIYPNASLLIQNIIQNDTGFYTLHVIKSDLVNEEATGQFRVYP corresponding to amino acids 1-142 of CEA5_HUMAN (SEQ ID NO:863), which also corresponds to amino acids 1-142 of HUMCEA_PEA_{—}1_P20 (SEQ ID NO:870), and a second amino acid sequence being at least 90% homologous to ELPKPSISSNNSKPVEDKDAVAFTCEPEAQNTTYLWWVNGQSLPVSPRLQLSNGNRTLTLFNVTRNDARA YVCGIQNSVSANRSDPVTLDVLYGPDTPIISPPDSSYLSGANLNLSCHSASNPSPQYSWRINGIPQQHTQVLF IAKITPNNNGTYACFVSNLATGRNNSIVKSITVSASGTSPGLSAGATVGIMIGVLVGVALI corresponding to amino acids 499-702 of CEA5_HUMAN (SEQ ID NO:863), which also corresponds to amino acids 143-346 of HUMCEA_PEA_{—}1_P20 (SEQ ID NO:870), wherein said first amino acid sequence and second amino acid sequence are contiguous and in a sequential order.

2. An isolated chimeric polypeptide encoding for an edge portion of HUMCEA_PEA_{—}1_P20 (SEQ ID NO:870), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise PE, having a structure as follows: a sequence starting from any of amino acid numbers 142−x to 142; and ending at any of amino acid numbers 143+((n−2)−x), in which x varies from 0 to n−2.

The location of the variant protein was determined according to results from a number of different software programs and analyses, including analyses from SignalP and other specialized programs. The variant protein is believed to be located as follows with regard to the cell: membrane. The protein localization is believed to be membrane because of manual inspection of known protein localization and/or gene structure.

Variant protein HUMCEA_PEA_{—}1_P20 (SEQ ID NO:870) also has the following non-silent SNPs (Single Nucleotide Polymorphisms) as listed in Table 25, (given according to their position(s) on the amino acid sequence, with the alternative amino acid(s) listed; the last column indicates whether the SNP is known or not; the presence of known SNPs in variant protein HUMCEA_PEA_{—}1_P20 (SEQ ID NO:870) sequence provides support for the deduced sequence of this variant protein according to the present invention).

TABLE 25
Amino acid mutations

| SNP position(s) on | | |
|---|---|---|
| amino acid sequence | Alternative amino acid(s) | Previously known SNP? |
| 63 | F -> L | No |
| 80 | I -> V | Yes |
| 83 | V -> A | Yes |
| 137 | Q -> P | Yes |
| 291 | P -> | No |
| 308 | R -> S | Yes |
| 326 | G -> | No |

The glycosylation sites of variant protein HUMCEA_PEA_{—}1_P20 (SEQ ID NO:870), as compared to the known protein Carcinoembryonic antigen-related cell adhesion molecule 5 precursor (SEQ ID NO:863), are described in Table 26 (given according to their position(s) on the amino acid sequence in the first column; the second column indicates whether the glycosylation site is present in the variant protein; and the last column indicates whether the position is different on the variant protein).

TABLE 26
Glycosylation site(s)

| Position(s) on known | Present in | Position |
|---|---|---|
| amino acid sequence | variant protein? | in variant protein? |
| 197 | no | |
| 466 | no | |
| 360 | no | |
| 288 | no | |
| 665 | yes | 309 |
| 560 | yes | 204 |
| 650 | yes | 294 |
| 480 | no | |
| 104 | yes | 104 |
| 580 | yes | 224 |
| 204 | no | |
| 115 | yes | 115 |
| 208 | no | |
| 152 | no | |
| 309 | no | |
| 432 | no | |
| 351 | no | |
| 246 | no | |
| 182 | no | |
| 612 | yes | 256 |
| 256 | no | |
| 508 | yes | 152 |
| 330 | no | |
| 274 | no | |
| 292 | no | |
| 553 | yes | 197 |
| 529 | yes | 173 |
| 375 | no | |

Variant protein HUMCEA_PEA_{—}1_P20 (SEQ ID NO:870) is encoded by the following transcript(s): HUMCEA_PEA_{—}1_T26 (SEQ ID NO:813), for which the sequence(s) is/are given at the end of the application. The coding portion of transcript HUMCEA_PEA_{—}1_T26 (SEQ ID NO:813) is shown in bold; this coding portion starts at position 115 and ends at position 1152. The transcript also has the following SNPs as listed in Table 27 (given according to their position on the nucleotide sequence, with the alternative nucleic acid listed; the last column indicates whether the SNP is known or not; the presence of known SNPs in variant protein HUMCEA_PEA_{—}1_P20 (SEQ ID NO:870) sequence provides support for the deduced sequence of this variant protein according to the present invention).

TABLE 27
Nucleic acid SNPs

| SNP position on | | |
|---|---|---|
| nucleotide sequence | Alternative nucleic acid | Previously known SNP? |
| 49 | T -> | No |
| 273 | A -> C | Yes |
| 303 | T -> G | No |
| 324 | T -> C | Yes |
| 352 | A -> G | Yes |
| 362 | T -> C | Yes |
| 524 | A -> C | Yes |
| 840 | T -> C | No |
| 894 | C -> T | No |
| 987 | A -> | No |
| 1036 | C -> A | Yes |
| 1090 | G -> | No |
| 1155 | G -> | No |
| 1171 | T -> A | No |
| 1172 | T -> A | No |
| 1364 | G -> A | Yes |
| 1417 | C -> T | No |
| 1928 | A -> G | Yes |
| 2094 | C -> T | Yes |
| 2285 | C -> T | Yes |

As noted above, cluster HUMCEA features 47 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HUMCEA_PEA_{—}1_node_{—}0 (SEQ ID NO:816) according to the present invention is supported by 56 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T16 (SEQ ID NO:810), HUMCEA_PEA_{—}1_T20 (SEQ ID NO:811), HUMCEA_PEA_{—}1_T25 (SEQ ID NO:812) and HUMCEA_PEA_{—}1_T26 (SEQ ID NO:813). Table 28 below describes the starting and ending position of this segment on each transcript.

TABLE 28
Segment location on transcripts

| | Segment | Segment |
|---|---|---|
| | starting | ending |
| Transcript name | position | position |
| HUMCEA_PEA_1_T8 (SEQ ID NO: 806) | 1 | 178 |
| HUMCEA_PEA_1_T9 (SEQ ID NO: 807) | 1 | 178 |
| HUMCEA_PEA_1_T12 (SEQ ID NO: 808) | 1 | 178 |
| HUMCEA_PEA_1_T16 (SEQ ID NO: 810) | 1 | 178 |
| HUMCEA_PEA_1_T20 (SEQ ID NO: 811) | 1 | 178 |
| HUMCEA_PEA_1_T25 (SEQ ID NO: 812) | 1 | 178 |
| HUMCEA_PEA_1_T26 (SEQ ID NO: 813) | 1 | 178 |

Segment cluster HUMCEA_PEA_{—}1_node_{—}2 (SEQ ID NO:817) according to the present invention is supported by 83 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T16 (SEQ ID NO:810), HUMCEA_PEA_{—}1_T20 (SEQ ID NO:811), HUMCEA_PEA_{—}1_T25 (SEQ ID NO:812) and HUMCEA_PEA_{—}1_T26 (SEQ ID NO:813). Table 29 below describes the starting and ending position of this segment on each transcript.

TABLE 29
Segment location on transcripts

| | Segment | Segment |
|---|---|---|
| | starting | ending |
| Transcript name | position | position |
| HUMCEA_PEA_1_T8 (SEQ ID NO: 806) | 179 | 456 |
| HUMCEA_PEA_1_T9 (SEQ ID NO: 807) | 179 | 456 |
| HUMCEA_PEA_1_T12 (SEQ ID NO: 808) | 179 | 456 |
| HUMCEA_PEA_1_T16 (SEQ ID NO: 810) | 179 | 456 |
| HUMCEA_PEA_1_T20 (SEQ ID NO: 811) | 179 | 456 |
| HUMCEA_PEA_1_T25 (SEQ ID NO: 812) | 179 | 456 |
| HUMCEA_PEA_1_T26 (SEQ ID NO: 813) | 179 | 456 |

Segment cluster HUMCEA_PEA_{—}1_node_{—}6 (SEQ ID NO:818) according to the present invention is supported by 8 libraries. The number of libraries was determined as previously described. This segment can be found in the following transcript(s): HUMCEA_PEA_{—}1_T14 (SEQ ID NO:809). Table 30 below describes the starting and ending position of this segment on each transcript.

TABLE 30
Segment location on transcripts

| | Segment | Segment |
|---|---|---|
| | starting | ending |
| Transcript name | position | position |
| HUMCEA_PEA_1_T14 (SEQ ID NO: 809) | 1 | 1258 |

Segment cluster HUMCEA_PEA_{—}1_node_{—}1 (SEQ ID NO:819) according to the present invention is supported by 6 libraries. The number of libraries was determined as previously described. This segment can be found in the following transcript(s): HUMCEA_PEA_{—}1_T8 (SEQ ID NO:806). Table 31 below describes the starting and ending position of this segment on each transcript.

TABLE 31
Segment location on transcripts

| | Segment | |
|---|---|---|
| | starting | Segment |
| Transcript name | position | ending position |
| HUMCEA_PEA_1_T8 (SEQ ID NO: 806) | 818 | 1217 |

Microarray (chip) data is also available for this segment as follows. As described above with regard to the cluster itself, various oligonucleotides were tested for being differentially expressed in various disease conditions, particularly cancer. The following oligonucleotides were found to hit this segment, shown in Table 32.

TABLE 32
Oligonucleotides related to this segment

| | Overexpressed | Chip |
|---|---|---|
| Oligonucleotide name | in cancers | reference |
| HUMCEA_0_0_96 (SEQ ID NO: 1428) | colorectal cancer | Colon |
| HUMCEA_0_0_96 (SEQ ID NO: 1428) | lung malignant | LUN |
| | tumors | |

Segment cluster HUMCEA_PEA_{—}1_node_{—}12 (SEQ ID NO:820) according to the present invention is supported by 83 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809) and HUMCEA_PEA_{—}1_T20 (SEQ ID NO:811). Table 33 below describes the starting and ending position of this segment on each transcript.

TABLE 33
Segment location on transcripts

| | Segment | Segment |
|---|---|---|
| | starting | ending |
| Transcript name | position | position |
| HUMCEA_PEA_1_T8 (SEQ ID NO: 806) | 1218 | 1472 |
| HUMCEA_PEA_1_T9 (SEQ ID NO: 807) | 818 | 1072 |
| HUMCEA_PEA_1_T12 (SEQ ID NO: 808) | 818 | 1072 |
| HUMCEA_PEA_1_T14 (SEQ ID NO: 809) | 1538 | 1792 |
| HUMCEA_PEA_1_T20 (SEQ ID NO: 811) | 818 | 1072 |

Segment cluster HUMCEA_PEA_{—}1_node_{—}31 (SEQ ID NO:821) according to the present invention is supported by 87 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810) and HUMCEA_PEA_{—}1_T20 (SEQ ID NO:811). Table 34 below describes the starting and ending position of this segment on each transcript.

TABLE 34
Segment location on transcripts

| | Segment | Segment |
|---|---|---|
| | starting | ending |
| Transcript name | position | position |
| HUMCEA_PEA_1_T8 (SEQ ID NO: 806) | 1817 | 2006 |
| HUMCEA_PEA_1_T9 (SEQ ID NO: 807) | 1417 | 1606 |
| HUMCEA_PEA_1_T12 (SEQ ID NO: 808) | 1417 | 1606 |
| HUMCEA_PEA_1_T14 (SEQ ID NO: 809) | 2137 | 2326 |
| HUMCEA_PEA_1_T16 (SEQ ID NO: 810) | 883 | 1072 |
| HUMCEA_PEA_1_T20 (SEQ ID NO: 811) | 1417 | 1606 |

Segment cluster HUMCEA_PEA_{—}1_node_{—}36 (SEQ ID NO:822) according to the present invention is supported by 94 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810) and HUMCEA_PEA_{—}1_T26 (SEQ ID NO:813). Table 35 below describes the starting and ending position of this segment on each transcript.

TABLE 35
Segment location on transcripts

| | Segment | Segment |
|---|---|---|
| | starting | ending |
| Transcript name | position | position |
| HUMCEA_PEA_1_T8 (SEQ ID NO: 806) | 2159 | 2285 |
| HUMCEA_PEA_1_T9 (SEQ ID NO: 807) | 1759 | 1885 |
| HUMCEA_PEA_1_T12 (SEQ ID NO: 808) | 1759 | 1885 |
| HUMCEA_PEA_1_T14 (SEQ ID NO: 809) | 2479 | 2605 |
| HUMCEA_PEA_1_T16 (SEQ ID NO: 810) | 1225 | 1351 |
| HUMCEA_PEA_1_T26 (SEQ ID NO: 813) | 691 | 817 |

Segment cluster HUMCEA_PEA_{—}1_node_{—}42 (SEQ ID NO:823) according to the present invention is supported by 5 libraries. The number of libraries was determined as previously described. This segment can be found in the following transcript(s): HUMCEA_PEA_{—}1_T29 (SEQ ID NO:814). Table 36 below describes the starting and ending position of this segment on each transcript.

TABLE 36
Segment location on transcripts

| | Segment | Segment |
|---|---|---|
| | starting | ending |
| Transcript name | position | position |
| HUMCEA_PEA_1_T29 (SEQ ID NO: 814) | 1 | 136 |

Segment cluster HUMCEA_PEA_{—}1_node_{—}43 (SEQ ID NO:824) according to the present invention is supported by 11 libraries. The number of libraries was determined as previously described. This segment can be found in the following transcript(s): HUMCEA_PEA_{—}1_T29 (SEQ ID NO:814). Table 37 below describes the starting and ending position of this segment on each transcript.

TABLE 37
Segment location on transcripts

| | Segment | Segment |
|---|---|---|
| | starting | ending |
| Transcript name | position | position |
| HUMCEA_PEA_1_T29 (SEQ ID NO: 814) | 137 | 260 |

Segment cluster HUMCEA_PEA_{—}1_node_{—}44 (SEQ ID NO:825) according to the present invention is supported by 112 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T25 (SEQ ID NO:812), HUMCEA_PEA_{—}1_T26 (SEQ ID NO:813) and HUMCEA_PEA_{—}1_T29 (SEQ ID NO:814). Table 38 below describes the starting and ending position of this segment on each transcript.

TABLE 38
Segment location on transcripts

| | Segment | Segment |
|---|---|---|
| | starting | ending |
| Transcript name | position | position |
| HUMCEA_PEA_1_T8 (SEQ ID NO: 806) | 2286 | 2540 |
| HUMCEA_PEA_1_T9 (SEQ ID NO: 807) | 1886 | 2140 |
| HUMCEA_PEA_1_T12 (SEQ ID NO: 808) | 1886 | 2140 |
| HUMCEA_PEA_1_T14 (SEQ ID NO: 809) | 2606 | 2860 |
| HUMCEA_PEA_1_T16 (SEQ ID NO: 810) | 1352 | 1606 |
| HUMCEA_PEA_1_T25 (SEQ ID NO: 812) | 818 | 1072 |
| HUMCEA_PEA_1_T26 (SEQ ID NO: 813) | 818 | 1072 |
| HUMCEA_PEA_1_T29 (SEQ ID NO: 814) | 261 | 515 |

Segment cluster HUMCEA_PEA_{—}1_node_{—}46 (SEQ ID NO:826) according to the present invention is supported by 15 libraries. The number of libraries was determined as previously described. This segment can be found in the following transcript(s): HUMCEA_PEA_{—}1_T9 (SEQ ID NO:807). Table 39 below describes the starting and ending position of this segment on each transcript.

TABLE 39
Segment location on transcripts

| | Segment | Segment |
|---|---|---|
| Transcript name | starting position | ending position |
| HUMCEA_PEA_1_T9 | 2174 | 3347 |
| (SEQ ID NO: 807) | | |

Segment cluster HUMCEA_PEA_{—}1_node_{—}48 (SEQ ID NO:827) according to the present invention is supported by 18 libraries. The number of libraries was determined as previously described. This segment can be found in the following transcript(s): HUMCEA_PEA_{—}1_T30 (SEQ ID NO:815). Table 40 below describes the starting and ending position of this segment on each transcript.

TABLE 40
Segment location on transcripts

| | Segment | Segment |
|---|---|---|
| Transcript name | starting position | ending position |
| HUMCEA_PEA_1_T30 | 1 | 1762 |
| (SEQ ID NO: 815) | | |

Segment cluster HUMCEA_PEA_{—}1_node_{—}63 (SEQ ID NO:828) according to the present invention is supported by 68 libraries. The number of libraries was determined as previously described. This segment can be found in the following transcript(s): HUMCEA_PEA_{—}1_T8 (SEQ ID NO:806), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T25 (SEQ ID NO:812), HUMCEA_PEA_{—}1_T26 (SEQ ID NO:813), HUMCEA_PEA_{—}1_T29 (SEQ ID NO:814) and HUMCEA_PEA_{—}1_T30 (SEQ ID NO:815). Table 41 below describes the starting and ending position of this segment on each transcript.

TABLE 41
Segment location on transcripts

| | Segment | Segment |
|---|---|---|
| | starting | ending |
| Transcript name | position | position |
| HUMCEA_PEA_1_T8 (SEQ ID NO: 806) | 2957 | 3135 |
| HUMCEA_PEA_1_T12 (SEQ ID NO: 808) | 2530 | 2708 |
| HUMCEA_PEA_1_T14 (SEQ ID NO: 809) | 3277 | 3455 |
| HUMCEA_PEA_1_T16 (SEQ ID NO: 810) | 2023 | 2201 |
| HUMCEA_PEA_1_T25 (SEQ ID NO: 812) | 1489 | 1667 |
| HUMCEA_PEA_1_T26 (SEQ ID NO: 813) | 1489 | 1667 |
| HUMCEA_PEA_1_T29 (SEQ ID NO: 814) | 932 | 1110 |
| HUMCEA_PEA_1_T30 (SEQ ID NO: 815) | 2185 | 2363 |

Segment cluster HUMCEA_PEA_{—}1_node_{—}65 (SEQ ID NO:829) according to the present invention is supported by 54 libraries. The number of libraries was determined as previously described. This segment can be found in the following transcript(s): HUMCEA_PEA_{—}1_T8 (SEQ ID NO:806), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T25 (SEQ ID NO:812), HUMCEA_PEA_{—}1_T26 (SEQ ID NO:813), HUMCEA_PEA_{—}1_T29 (SEQ ID NO:814) and HUMCEA_PEA_{—}1_T30 (SEQ ID NO:815). Table 42 below describes the starting and ending position of this segment on each transcript.

TABLE 42
Segment location on transcripts

| | Segment | Segment |
|---|---|---|
| | starting | ending |
| Transcript name | position | position |
| HUMCEA_PEA_1_T8 (SEQ ID NO: 806) | 3166 | 3897 |
| HUMCEA_PEA_1_T12 (SEQ ID NO: 808) | 2739 | 3470 |
| HUMCEA_PEA_1_T14 (SEQ ID NO: 809) | 3486 | 4217 |
| HUMCEA_PEA_1_T16 (SEQ ID NO: 810) | 2232 | 2963 |
| HUMCEA_PEA_1_T25 (SEQ ID NO: 812) | 1698 | 2429 |
| HUMCEA_PEA_1_T26 (SEQ ID NO: 813) | 1698 | 2429 |
| HUMCEA_PEA_1_T29 (SEQ ID NO: 814) | 1141 | 1872 |
| HUMCEA_PEA_1_T30 (SEQ ID NO: 815) | 2394 | 3125 |

Segment cluster HUMCEA_PEA_{—}1_node_{—}67 (SEQ ID NO:830) according to the present invention is supported by 2 libraries. The number of libraries was determined as previously described. This segment can be found in the following transcript(s): HUMCEA_PEA_{—}1_T20 (SEQ ID NO:811). Table 43 below describes the starting and ending position of this segment on each transcript.

TABLE 43
Segment location on transcripts

| | Segment | Segment |
|---|---|---|
| Transcript name | starting position | ending position |
| HUMCEA_PEA_1_T20 | 1607 | 1886 |
| (SEQ ID NO: 811) | | |

According to an optional embodiment of the present invention, short segments related to the above cluster are also provided. These segments are up to about 120 bp in length, and so are included in a separate description.

Segment cluster HUMCEA_PEA_{—}1_node_{—}3 (SEQ ID NO:831) according to the present invention is supported by 67 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T16 (SEQ ID NO:810), HUMCEA_PEA_{—}1_T20 (SEQ ID NO:811), HUMCEA_PEA_{—}1_T25 (SEQ ID NO:812) and HUMCEA_PEA_{—}1_T26 (SEQ ID NO:813). Table 44 below describes the starting and ending position of this segment on each transcript.

TABLE 44
Segment location on transcripts

| | Segment | Segment |
|---|---|---|
| | starting | ending |
| Transcript name | position | position |
| HUMCEA_PEA_1_T8 (SEQ ID NO: 806) | 457 | 538 |
| HUMCEA_PEA_1_T9 (SEQ ID NO: 807) | 457 | 538 |
| HUMCEA_PEA_1_T12 (SEQ ID NO: 808) | 457 | 538 |
| HUMCEA_PEA_1_T16 (SEQ ID NO: 810) | 457 | 538 |
| HUMCEA_PEA_1_T20 (SEQ ID NO: 811) | 457 | 538 |
| HUMCEA_PEA_1_T25 (SEQ ID NO: 812) | 457 | 538 |
| HUMCEA_PEA_1_T26 (SEQ ID NO: 813) | 457 | 538 |

Segment cluster HUMCEA_PEA_{—}1_node_{—}7 (SEQ ID NO:832) according to the present invention is supported by 73 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T20 (SEQ ID NO:811) and HUMCEA_PEA_{—}1_T25 (SEQ ID NO:812). Table 45 below describes the starting and ending position of this segment on each transcript.

TABLE 45
Segment location on transcripts

| | Segment | Segment |
|---|---|---|
| | starting | ending |
| Transcript name | position | position |
| HUMCEA_PEA_1_T8 (SEQ ID NO: 806) | 539 | 642 |
| HUMCEA_PEA_1_T9 (SEQ ID NO: 807) | 539 | 642 |
| HUMCEA_PEA_1_T12 (SEQ ID NO: 808) | 539 | 642 |
| HUMCEA_PEA_1_T14 (SEQ ID NO: 809) | 1259 | 1362 |
| HUMCEA_PEA_1_T16 (SEQ ID NO: 810) | 539 | 642 |
| HUMCEA_PEA_1_T20 (SEQ ID NO: 811) | 539 | 642 |
| HUMCEA_PEA_1_T25 (SEQ ID NO: 812) | 539 | 642 |

Segment cluster HUMCEA_PEA_{—}1_node_{—}8 (SEQ ID NO:833) according to the present invention is supported by 67 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T20 (SEQ ID NO:811) and HUMCEA_PEA_{—}1_T25 (SEQ ID NO:812). Table 46 below describes the starting and ending position of this segment on each transcript.

TABLE 46
Segment location on transcripts

| | Segment | Segment |
|---|---|---|
| | starting | ending |
| Transcript name | position | position |
| HUMCEA_PEA_1_T8 (SEQ ID NO: 806) | 643 | 690 |
| HUMCEA_PEA_1_T9 (SEQ ID NO: 807) | 643 | 690 |
| HUMCEA_PEA_1_T12 (SEQ ID NO: 808) | 643 | 690 |
| HUMCEA_PEA_1_T14 (SEQ ID NO: 809) | 1363 | 1410 |
| HUMCEA_PEA_1_T16 (SEQ ID NO: 810) | 643 | 690 |
| HUMCEA_PEA_1_T20 (SEQ ID NO: 811) | 643 | 690 |
| HUMCEA_PEA_1_T25 (SEQ ID NO: 812) | 643 | 690 |

Segment cluster HUMCEA_PEA_{—}1_node_{—}9 (SEQ ID NO:834) according to the present invention is supported by 71 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T20 (SEQ ID NO:811) and HUMCEA_PEA_{—}1_T25 (SEQ ID NO:812). Table 47 below describes the starting and ending position of this segment on each transcript.

TABLE 47
Segment location on transcripts

| | Segment | Segment |
|---|---|---|
| | starting | ending |
| Transcript name | position | position |
| HUMCEA_PEA_1_T8 (SEQ ID NO: 806) | 691 | 738 |
| HUMCEA_PEA_1_T9 (SEQ ID NO: 807) | 691 | 738 |
| HUMCEA_PEA_1_T12 (SEQ ID NO: 808) | 691 | 738 |
| HUMCEA_PEA_1_T14 (SEQ ID NO: 809) | 1411 | 1458 |
| HUMCEA_PEA_1_T16 (SEQ ID NO: 810) | 691 | 738 |
| HUMCEA_PEA_1_T20 (SEQ ID NO: 811) | 691 | 738 |
| HUMCEA_PEA_1_T25 (SEQ ID NO: 812) | 691 | 738 |

Segment cluster HUMCEA_PEA_{—}1_node_{—}10 (SEQ ID NO:835) according to the present invention is supported by 67 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T20 (SEQ ID NO:811) and HUMCEA_PEA_{—}1_T25 (SEQ ID NO:812). Table 48 below describes the starting and ending position of this segment on each transcript.

TABLE 48
Segment location on transcripts

| | Segment | Segment |
|---|---|---|
| | starting | ending |
| Transcript name | position | position |
| HUMCEA_PEA_1_T8 (SEQ ID NO: 806) | 739 | 817 |
| HUMCEA_PEA_1_T9 (SEQ ID NO: 807) | 739 | 817 |
| HUMCEA_PEA_1_T12 (SEQ ID NO: 808) | 739 | 817 |
| HUMCEA_PEA_1_T14 (SEQ ID NO: 809) | 1459 | 1537 |
| HUMCEA_PEA_1_T16 (SEQ ID NO: 810) | 739 | 817 |
| HUMCEA_PEA_1_T20 (SEQ ID NO: 811) | 739 | 817 |
| HUMCEA_PEA_1_T25 (SEQ ID NO: 812) | 739 | 817 |

Segment cluster HUMCEA_PEA_{—}1_node_{—}15 (SEQ ID NO:836) according to the present invention can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809) and HUMCEA_PEA_{—}1_T20 (SEQ ID NO:811). Table 49 below describes the starting and ending position of this segment on each transcript.

TABLE 49
Segment location on transcripts

| | Segment | Segment |
|---|---|---|
| | starting | ending |
| Transcript name | position | position |
| HUMCEA_PEA_1_T8 (SEQ ID NO: 806) | 1473 | 1475 |
| HUMCEA_PEA_1_T9 (SEQ ID NO: 807) | 1073 | 1075 |
| HUMCEA_PEA_1_T12 (SEQ ID NO: 808) | 1073 | 1075 |
| HUMCEA_PEA_1_T14 (SEQ ID NO: 809) | 1793 | 1795 |
| HUMCEA_PEA_1_T20 (SEQ ID NO: 811) | 1073 | 1075 |

Segment cluster HUMCEA_PEA_{—}1_node_{—}16 (SEQ ID NO:837) according to the present invention can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809) and HUMCEA_PEA_{—}1_T20 (SEQ ID NO:811). Table 50 below describes the starting and ending position of this segment on each transcript.

TABLE 50
Segment location on transcripts

| | Segment | Segment |
|---|---|---|
| | starting | ending |
| Transcript name | position | position |
| HUMCEA_PEA_1_T8 (SEQ ID NO: 806) | 1476 | 1481 |
| HUMCEA_PEA_1_T9 (SEQ ID NO: 807) | 1076 | 1081 |
| HUMCEA_PEA_1_T12 (SEQ ID NO: 808) | 1076 | 1081 |
| HUMCEA_PEA_1_T14 (SEQ ID NO: 809) | 1796 | 1801 |
| HUMCEA_PEA_1_T20 (SEQ ID NO: 811) | 1076 | 1081 |

Segment cluster HUMCEA_PEA_{—}1_node_{—}17 (SEQ ID NO:838) according to the present invention can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809) and HUMCEA_PEA_{—}1_T20 (SEQ ID NO:811). Table 51 below describes the starting and ending position of this segment on each transcript.

TABLE 51
Segment location on transcripts

| | Segment | Segment |
|---|---|---|
| | starting | ending |
| Transcript name | position | position |
| HUMCEA_PEA_1_T8 (SEQ ID NO: 806) | 1482 | 1488 |
| HUMCEA_PEA_1_T9 (SEQ ID NO: 807) | 1082 | 1088 |
| HUMCEA_PEA_1_T12 (SEQ ID NO: 808) | 1082 | 1088 |
| HUMCEA_PEA_1_T14 (SEQ ID NO: 809) | 1802 | 1808 |
| HUMCEA_PEA_1_T20 (SEQ ID NO: 811) | 1082 | 1088 |

Segment cluster HUMCEA_PEA_{—}1_node_{—}18 (SEQ ID NO:839) according to the present invention can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809) and HUMCEA_PEA_{—}1_T20 (SEQ ID NO:811). Table 52 below describes the starting and ending position of this segment on each transcript.

TABLE 52
Segment location on transcripts

| | Segment | Segment |
|---|---|---|
| | starting | ending |
| Transcript name | position | position |
| HUMCEA_PEA_1_T8 (SEQ ID NO: 806) | 1489 | 1506 |
| HUMCEA_PEA_1_T9 (SEQ ID NO: 807) | 1089 | 1106 |
| HUMCEA_PEA_1_T12 (SEQ ID NO: 808) | 1089 | 1106 |
| HUMCEA_PEA_1_T14 (SEQ ID NO: 809) | 1809 | 1826 |
| HUMCEA_PEA_1_T20 (SEQ ID NO: 811) | 1089 | 1106 |

Segment cluster HUMCEA_PEA_{—}1_node_{—}19 (SEQ ID NO:840) according to the present invention is supported by 69 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809) and HUMCEA_PEA_{—}1_T20 (SEQ ID NO:811). Table 53 below describes the starting and ending position of this segment on each transcript.

TABLE 53
Segment location on transcripts

| | Segment | Segment |
|---|---|---|
| | starting | ending |
| Transcript name | position | position |
| HUMCEA_PEA_1_T8 (SEQ ID NO: 806) | 1507 | 1576 |
| HUMCEA_PEA_1_T9 (SEQ ID NO: 807) | 1107 | 1176 |
| HUMCEA_PEA_1_T12 (SEQ ID NO: 808) | 1107 | 1176 |
| HUMCEA_PEA_1_T14 (SEQ ID NO: 809) | 1827 | 1896 |
| HUMCEA_PEA_1_T20 (SEQ ID NO: 811) | 1107 | 1176 |

Segment cluster HUMCEA_PEA_{—}1_node_{—}20 (SEQ ID NO:841) according to the present invention can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809) and HUMCEA_PEA_{—}1_T20 (SEQ ID NO:811). Table 54 below describes the starting and ending position of this segment on each transcript.

TABLE 54
Segment location on transcripts

| | Segment | Segment |
|---|---|---|
| | starting | ending |
| Transcript name | position | position |
| HUMCEA_PEA_1_T8 (SEQ ID NO: 806) | 1577 | 1600 |
| HUMCEA_PEA_1_T9 (SEQ ID NO: 807) | 1177 | 1200 |
| HUMCEA_PEA_1_T12 (SEQ ID NO: 808) | 1177 | 1200 |
| HUMCEA_PEA_1_T14 (SEQ ID NO: 809) | 1897 | 1920 |
| HUMCEA_PEA_1_T20 (SEQ ID NO: 811) | 1177 | 1200 |

Segment cluster HUMCEA_PEA_{—}1_node_{—}21 (SEQ ID NO:842) according to the present invention can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809) and HUMCEA_PEA_{—}1_T20 (SEQ ID NO:811). Table 55 below describes the starting and ending position of this segment on each transcript.

TABLE 55
Segment location on transcripts

| | Segment | Segment |
|---|---|---|
| | starting | ending |
| Transcript name | position | position |
| HUMCEA_PEA_1_T8 (SEQ ID NO: 806) | 1601 | 1624 |
| HUMCEA_PEA_1_T9 (SEQ ID NO: 807) | 1201 | 1224 |
| HUMCEA_PEA_1_T12 (SEQ ID NO: 808) | 1201 | 1224 |
| HUMCEA_PEA_1_T14 (SEQ ID NO: 809) | 1921 | 1944 |
| HUMCEA_PEA_1_T20 (SEQ ID NO: 811) | 1201 | 1224 |

Segment cluster HUMCEA_PEA_{—}1_node_{—}22 (SEQ ID NO:843) according to the present invention is supported by 77 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809) and HUMCEA_PEA_{—}1_T20 (SEQ ID NO:811). Table 56 below describes the starting and ending position of this segment on each transcript.

TABLE 56
Segment location on transcripts

| | Segment | Segment |
|---|---|---|
| | starting | ending |
| Transcript name | position | position |
| HUMCEA_PEA_1_T8 (SEQ ID NO: 806) | 1625 | 1702 |
| HUMCEA_PEA_1_T9 (SEQ ID NO: 807) | 1225 | 1302 |
| HUMCEA_PEA_1_T12 (SEQ ID NO: 808) | 1225 | 1302 |
| HUMCEA_PEA_1_T14 (SEQ ID NO: 809) | 1945 | 2022 |
| HUMCEA_PEA_1_T20 (SEQ ID NO: 811) | 1225 | 1302 |

Segment cluster HUMCEA_PEA_{—}1_node_{—}23 (SEQ ID NO:844) according to the present invention is supported by 72 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809) and HUMCEA_PEA_{—}1_T20 (SEQ ID NO:811). Table 57 below describes the starting and ending position of this segment on each transcript.

TABLE 57
Segment location on transcripts

| | Segment | Segment |
|---|---|---|
| | starting | ending |
| Transcript name | position | position |
| HUMCEA_PEA_1_T8 (SEQ ID NO: 806) | 1703 | 1732 |
| HUMCEA_PEA_1_T9 (SEQ ID NO: 807) | 1303 | 1332 |
| HUMCEA_PEA_1_T12 (SEQ ID NO: 808) | 1303 | 1332 |
| HUMCEA_PEA_1_T14 (SEQ ID NO: 809) | 2023 | 2052 |
| HUMCEA_PEA_1_T20 (SEQ ID NO: 811) | 1303 | 1332 |

Segment cluster HUMCEA_PEA_{—}1_node_{—}24 (SEQ ID NO:845) according to the present invention can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809) and HUMCEA_PEA_{—}1_T20 (SEQ ID NO:811). Table 58 below describes the starting and ending position of this segment on each transcript.

TABLE 58
Segment location on transcripts

| | Segment | Segment |
|---|---|---|
| | starting | ending |
| Transcript name | position | position |
| HUMCEA_PEA_1_T8 (SEQ ID NO: 806) | 1733 | 1751 |
| HUMCEA_PEA_1_T9 (SEQ ID NO: 807) | 1333 | 1351 |
| HUMCEA_PEA_1_T12 (SEQ ID NO: 808) | 1333 | 1351 |
| HUMCEA_PEA_1_T14 (SEQ ID NO: 809) | 2053 | 2071 |
| HUMCEA_PEA_1_T20 (SEQ ID NO: 811) | 1333 | 1351 |

Segment cluster HUMCEA_PEA_{—}1_node_{—}27 (SEQ ID NO:846) according to the present invention can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810) and HUMCEA_PEA_{—}1_T20 (SEQ ID NO:811). Table 59 below describes the starting and ending position of this segment on each transcript.

TABLE 59
Segment location on transcripts

| | Segment | Segment |
|---|---|---|
| | starting | ending |
| Transcript name | position | position |
| HUMCEA_PEA_1_T8 (SEQ ID NO: 806) | 1752 | 1770 |
| HUMCEA_PEA_1_T9 (SEQ ID NO: 807) | 1352 | 1370 |
| HUMCEA_PEA_1_T12 (SEQ ID NO: 808) | 1352 | 1370 |
| HUMCEA_PEA_1_T14 (SEQ ID NO: 809) | 2072 | 2090 |
| HUMCEA_PEA_1_T16 (SEQ ID NO: 810) | 818 | 836 |
| HUMCEA_PEA_1_T20 (SEQ ID NO: 811) | 1352 | 1370 |

Segment cluster HUMCEA_PEA_{—}1_node_{—}29 (SEQ ID NO:847) according to the present invention can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810) and HUMCEA_PEA_{—}1_T20 (SEQ ID NO:811). Table 60 below describes the starting and ending position of this segment on each transcript.

TABLE 60
Segment location on transcripts

| | Segment | Segment |
|---|---|---|
| | starting | ending |
| Transcript name | position | position |
| HUMCEA_PEA_1_T8 (SEQ ID NO: 806) | 1771 | 1788 |
| HUMCEA_PEA_1_T9 (SEQ ID NO: 807) | 1371 | 1388 |
| HUMCEA_PEA_1_T12 (SEQ ID NO: 808) | 1371 | 1388 |
| HUMCEA_PEA_1_T14 (SEQ ID NO: 809) | 2091 | 2108 |
| HUMCEA_PEA_1_T16 (SEQ ID NO: 810) | 837 | 854 |
| HUMCEA_PEA_1_T20 (SEQ ID NO: 811) | 1371 | 1388 |

Segment cluster HUMCEA_PEA_{—}1_node_{—}30 (SEQ ID NO:848) according to the present invention is supported by 67 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810) and HUMCEA_PEA_{—}1_T20 (SEQ ID NO:811). Table 61 below describes the starting and ending position of this segment on each transcript.

TABLE 61
Segment location on transcripts

| | Segment | Segment |
|---|---|---|
| | starting | ending |
| Transcript name | position | position |
| HUMCEA_PEA_1_T8 (SEQ ID NO: 806) | 1789 | 1816 |
| HUMCEA_PEA_1_T9 (SEQ ID NO: 807) | 1389 | 1416 |
| HUMCEA_PEA_1_T12 (SEQ ID NO: 808) | 1389 | 1416 |
| HUMCEA_PEA_1_T14 (SEQ ID NO: 809) | 2109 | 2136 |
| HUMCEA_PEA_1_T16 (SEQ ID NO: 810) | 855 | 882 |
| HUMCEA_PEA_1_T20 (SEQ ID NO: 811) | 1389 | 1416 |

Segment cluster HUMCEA_PEA_{—}1_node_{—}33 (SEQ ID NO:849) according to the present invention can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810) and HUMCEA_PEA_{—}1_T26 (SEQ ID NO:813). Table 62 below describes the starting and ending position of this segment on each transcript.

TABLE 62
Segment location on transcripts

| | Segment | Segment |
|---|---|---|
| | starting | ending |
| Transcript name | position | position |
| HUMCEA_PEA_1_T8 (SEQ ID NO: 806) | 2007 | 2028 |
| HUMCEA_PEA_1_T9 (SEQ ID NO: 807) | 1607 | 1628 |
| HUMCEA_PEA_1_T12 (SEQ ID NO: 808) | 1607 | 1628 |
| HUMCEA_PEA_1_T14 (SEQ ID NO: 809) | 2327 | 2348 |
| HUMCEA_PEA_1_T16 (SEQ ID NO: 810) | 1073 | 1094 |
| HUMCEA_PEA_1_T26 (SEQ ID NO: 813) | 539 | 560 |

Segment cluster HUMCEA_PEA_{—}1_node_{—}34 (SEQ ID NO:850) according to the present invention is supported by 80 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810) and HUMCEA_PEA_{—}1_T26 (SEQ ID NO:813). Table 63 below describes the starting and ending position of this segment on each transcript.

TABLE 63
Segment location on transcripts

| | Segment | Segment |
|---|---|---|
| | starting | ending |
| Transcript name | position | position |
| HUMCEA_PEA_1_T8 (SEQ ID NO: 806) | 2029 | 2110 |
| HUMCEA_PEA_1_T9 (SEQ ID NO: 807) | 1629 | 1710 |
| HUMCEA_PEA_1_T12 (SEQ ID NO: 808) | 1629 | 1710 |
| HUMCEA_PEA_1_T14 (SEQ ID NO: 809) | 2349 | 2430 |
| HUMCEA_PEA_1_T16 (SEQ ID NO: 810) | 1095 | 1176 |
| HUMCEA_PEA_1_T26 (SEQ ID NO: 813) | 561 | 642 |

Segment cluster HUMCEA_PEA_{—}1_node_{—}35 (SEQ ID NO:851) according to the present invention is supported by 75 libraries. The number of libraries was determined as previously described. This segment can be found in the following transcript(s): HUMCEA_PEA_{—}1_T8 (SEQ ID NO:806), HUMCEA_PEA_{—}1_T9 (SEQ ID NO:807), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810) and HUMCEA_PEA_{—}1_T26 (SEQ ID NO:813). Table 64 below describes the starting and ending position of this segment on each transcript.

TABLE 64
Segment location on transcripts

| | Segment | Segment |
|---|---|---|
| | starting | ending |
| Transcript name | position | position |
| HUMCEA_PEA_1_T8 (SEQ ID NO: 806) | 2111 | 2158 |
| HUMCEA_PEA_1_T9 (SEQ ID NO: 807) | 1711 | 1758 |
| HUMCEA_PEA_1_T12 (SEQ ID NO: 808) | 1711 | 1758 |
| HUMCEA_PEA_1_T14 (SEQ ID NO: 809) | 2431 | 2478 |
| HUMCEA_PEA_1_T16 (SEQ ID NO: 810) | 1177 | 1224 |
| HUMCEA_PEA_1_T26 (SEQ ID NO: 813) | 643 | 690 |

Segment cluster HUMCEA_PEA_{—}1_node_{—}45 (SEQ ID NO:852) according to the present invention is supported by 9 libraries. The number of libraries was determined as previously described. This segment can be found in the following transcript(s): HUMCEA_PEA_{—}1_T9 (SEQ ID NO:807). Table 65 below describes the starting and ending position of this segment on each transcript.

TABLE 65
Segment location on transcripts

| | Segment | Segment |
|---|---|---|
| | starting | ending |
| Transcript name | position | position |
| HUMCEA_PEA_1_T9 (SEQ ID NO: 807) | 2141 | 2173 |

Segment cluster HUMCEA_PEA_{—}1_node_{—}49 (SEQ ID NO:853) according to the present invention can be found in the following transcript(s): HUMCEA_PEA_{—}1_T30 (SEQ ID NO:815). Table 66 below describes the starting and ending position of this segment on each transcript.

TABLE 66
Segment location on transcripts

| | Segment | Segment |
|---|---|---|
| | starting | ending |
| Transcript name | position | position |
| HUMCEA_PEA_1_T30 (SEQ ID NO: 815) | 1763 | 1768 |

Segment cluster HUMCEA_PEA_{—}1_node_{—}50 (SEQ ID NO:854) according to the present invention is supported by 64 libraries. The number of libraries was determined as previously described. This segment can be found in the following transcript(s): HUMCEA_PEA_{—}1_T8 (SEQ ID NO:806), HUMCEA_PEA_{—}1_T14 (SEQ ID NO:809), HUMCEA_PEA_{—}1_T16 (SEQ ID NO:810), HUMCEA_PEA_{—}1_T25 (SEQ ID NO:812), HUMCEA_PEA_{—}1_T26 (SEQ ID NO:813), HUMCEA_PEA_{—}1_T29 (SEQ ID NO:814) and HUMCEA_PEA_{—}1_T30 (SEQ ID NO:815). Table 67 below describes the starting and ending position of this segment on each transcript.

TABLE 67
Segment location on transcripts

| | Segment | Segment |
|---|---|---|
| | starting | ending |
| Transcript name | position | position |
| HUMCEA_PEA_1_T8 (SEQ ID NO: 806) | 2541 | 2567 |
| HUMCEA_PEA_1_T14 (SEQ ID NO: 809) | 2861 | 2887 |
| HUMCEA_PEA_1_T16 (SEQ ID NO: 810) | 1607 | 1633 |
| HUMCEA_PEA_1_T25 (SEQ ID NO: 812) | 1073 | 1099 |
| HUMCEA_PEA_1_T26 (SEQ ID NO: 813) | 1073 | 1099 |
| HUMCEA_PEA_1_T29 (SEQ ID NO: 814) | 516 | 542 |
| HUMCEA_PEA_1_T30 (SEQ ID NO: 815) | 1769 | 1795 |

Segment cluster HUMCEA_PEA_{—}1_node_{—}51 (SEQ ID NO:855) according to the present invention is supported by 88 libraries. The number of libraries was determined as previously described. This segment can be found in the following transcript(s): HUMCEA_PEA_{—}1_T8 (SEQ ID NO:806), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T25 (SEQ ID NO:812), HUMCEA_PEA_{—}1_T26 (SEQ ID NO:813), HUMCEA_PEA_{—}1_T29 (SEQ ID NO:814) and HUMCEA_PEA_{—}1_T30 (SEQ ID NO:815). Table 68 below describes the starting and ending position of this segment on each transcript.

TABLE 68
Segment location on transcripts

| | Segment | Segment |
|---|---|---|
| | starting | ending |
| Transcript name | position | position |
| HUMCEA_PEA_1_T8 (SEQ ID NO: 806) | 2568 | 2659 |
| HUMCEA_PEA_1_T12 (SEQ ID NO: 808) | 2141 | 2232 |
| HUMCEA_PEA_1_T14 (SEQ ID NO: 809) | 2888 | 2979 |
| HUMCEA_PEA_1_T16 (SEQ ID NO: 810) | 1634 | 1725 |
| HUMCEA_PEA_1_T25 (SEQ ID NO: 812) | 1100 | 1191 |
| HUMCEA_PEA_1_T26 (SEQ ID NO: 813) | 1100 | 1191 |
| HUMCEA_PEA_1_T29 (SEQ ID NO: 814) | 543 | 634 |
| HUMCEA_PEA_1_T30 (SEQ ID NO: 815) | 1796 | 1887 |

Segment cluster HUMCEA_PEA_{—}1_node_{—}56 (SEQ ID NO:856) according to the present invention is supported by 75 libraries. The number of libraries was determined as previously described. This segment can be found in the following transcript(s): HUMCEA_PEA_{—}1_T8 (SEQ ID NO:806), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T25 (SEQ ID NO:812), HUMCEA_PEA_{—}1_T26 (SEQ ID NO:813), HUMCEA_PEA_{—}1_T29 (SEQ ID NO:814) and HUMCEA_PEA_{—}1_T30 (SEQ ID NO:815). Table 69 below describes the starting and ending position of this segment on each transcript.

TABLE 69
Segment location on transcripts

| | Segment | Segment |
|---|---|---|
| | starting | ending |
| Transcript name | position | position |
| HUMCEA_PEA_1_T8 (SEQ ID NO: 806) | 2660 | 2685 |
| HUMCEA_PEA_1_T12 (SEQ ID NO: 808) | 2233 | 2258 |
| HUMCEA_PEA_1_T14 (SEQ ID NO: 809) | 2980 | 3005 |
| HUMCEA_PEA_1_T16 (SEQ ID NO: 810) | 1726 | 1751 |
| HUMCEA_PEA_1_T25 (SEQ ID NO: 812) | 1192 | 1217 |
| HUMCEA_PEA_1_T26 (SEQ ID NO: 813) | 1192 | 1217 |
| HUMCEA_PEA_1_T29 (SEQ ID NO: 814) | 635 | 660 |
| HUMCEA_PEA_1_T30 (SEQ ID NO: 815) | 1888 | 1913 |

Segment cluster HUMCEA_PEA_{—}1_node_{—}57 (SEQ ID NO:857) according to the present invention is supported by 82 libraries. The number of libraries was determined as previously described. This segment can be found in the following transcript(s): HUMCEA_PEA_{—}1_T8 (SEQ ID NO:806), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T25 (SEQ ID NO:812), HUMCEA_PEA_{—}1_T26 (SEQ ID NO:813), HUMCEA_PEA_{—}1_T29 (SEQ ID NO:814) and HUMCEA_PEA_{—}1_T30 (SEQ ID NO:815). Table 70 below describes the starting and ending position of this segment on each transcript.

TABLE 70
Segment location on transcripts

| | Segment | Segment |
|---|---|---|
| | starting | ending |
| Transcript name | position | position |
| HUMCEA_PEA_1_T8 (SEQ ID NO: 806) | 2686 | 2786 |
| HUMCEA_PEA_1_T12 (SEQ ID NO: 808) | 2259 | 2359 |
| HUMCEA_PEA_1_T14 (SEQ ID NO: 809) | 3006 | 3106 |
| HUMCEA_PEA_1_T16 (SEQ ID NO: 810) | 1752 | 1852 |
| HUMCEA_PEA_1_T25 (SEQ ID NO: 812) | 1218 | 1318 |
| HUMCEA_PEA_1_T26 (SEQ ID NO: 813) | 1218 | 1318 |
| HUMCEA_PEA_1_T29 (SEQ ID NO: 814) | 661 | 761 |
| HUMCEA_PEA_1_T30 (SEQ ID NO: 815) | 1914 | 2014 |

Segment cluster HUMCEA_PEA_{—}1_node_{—}58 (SEQ ID NO:858) according to the present invention is supported by 63 libraries. The number of libraries was determined as previously described. This segment can be found in the following transcript(s): HUMCEA_PEA_{—}1_T8 (SEQ ID NO:806), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T25 (SEQ ID NO:812), HUMCEA_PEA_{—}1_T26 (SEQ ID NO:813), HUMCEA_PEA_{—}1_T29 (SEQ ID NO:814) and HUMCEA_PEA_{—}1_T30 (SEQ ID NO:815). Table 71 below describes the starting and ending position of this segment on each transcript.

TABLE 71
Segment location on transcripts

| | Segment | Segment |
|---|---|---|
| | starting | ending |
| Transcript name | position | position |
| HUMCEA_PEA_1_T8 (SEQ ID NO: 806) | 2787 | 2820 |
| HUMCEA_PEA_1_T12 (SEQ ID NO: 808) | 2360 | 2393 |
| HUMCEA_PEA_1_T14 (SEQ ID NO: 809) | 3107 | 3140 |
| HUMCEA_PEA_1_T16 (SEQ ID NO: 810) | 1853 | 1886 |
| HUMCEA_PEA_1_T25 (SEQ ID NO: 812) | 1319 | 1352 |
| HUMCEA_PEA_1_T26 (SEQ ID NO: 813) | 1319 | 1352 |
| HUMCEA_PEA_1_T29 (SEQ ID NO: 814) | 762 | 795 |
| HUMCEA_PEA_1_T30 (SEQ ID NO: 815) | 2015 | 2048 |

Segment cluster HUMCEA_PEA_{—}1_node_{—}60 (SEQ ID NO:859) according to the present invention is supported by 55 libraries. The number of libraries was determined as previously described. This segment can be found in the following transcript(s): HUMCEA_PEA_{—}1_T8 (SEQ ID NO:806), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T25 (SEQ ID NO:812), HUMCEA_PEA_{—}1_T26 (SEQ ID NO:813), HUMCEA_PEA_{—}1_T29 (SEQ ID NO:814) and HUMCEA_PEA_{—}1_T30 (SEQ ID NO:815). Table 72 below describes the starting and ending position of this segment on each transcript.

TABLE 72
Segment location on transcripts

| | Segment | Segment |
|---|---|---|
| | starting | ending |
| Transcript name | position | position |
| HUMCEA_PEA_1_T8 (SEQ ID NO: 806) | 2821 | 2864 |
| HUMCEA_PEA_1_T12 (SEQ ID NO: 808) | 2394 | 2437 |
| HUMCEA_PEA_1_T14 (SEQ ID NO: 809) | 3141 | 3184 |
| HUMCEA_PEA_1_T16 (SEQ ID NO: 810) | 1887 | 1930 |
| HUMCEA_PEA_1_T25 (SEQ ID NO: 812) | 1353 | 1396 |
| HUMCEA_PEA_1_T26 (SEQ ID NO: 813) | 1353 | 1396 |
| HUMCEA_PEA_1_T29 (SEQ ID NO: 814) | 796 | 839 |
| HUMCEA_PEA_1_T30 (SEQ ID NO: 815) | 2049 | 2092 |

Segment cluster HUMCEA_PEA_{—}1_node_{—}61 (SEQ ID NO:860) according to the present invention can be found in the following transcript(s): HUMCEA_PEA_{—}1_T8 (SEQ ID NO:806), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T25 (SEQ ID NO:812), HUMCEA_PEA_{—}1_T26 (SEQ ID NO:813), HUMCEA_PEA_{—}1_T29 (SEQ ID NO:814) and HUMCEA_PEA_{—}1_T30 (SEQ ID NO:815). Table 73 below describes the starting and ending position of this segment on each transcript.

TABLE 73
Segment location on transcripts

| | Segment | Segment |
|---|---|---|
| | starting | ending |
| Transcript name | position | position |
| HUMCEA_PEA_1_T8 (SEQ ID NO: 806) | 2865 | 2868 |
| HUMCEA_PEA_1_T12 (SEQ ID NO: 808) | 2438 | 2441 |
| HUMCEA_PEA_1_T14 (SEQ ID NO: 809) | 3185 | 3188 |
| HUMCEA_PEA_1_T16 (SEQ ID NO: 810) | 1931 | 1934 |
| HUMCEA_PEA_1_T25 (SEQ ID NO: 812) | 1397 | 1400 |
| HUMCEA_PEA_1_T26 (SEQ ID NO: 813) | 1397 | 1400 |
| HUMCEA_PEA_1_T29 (SEQ ID NO: 814) | 840 | 843 |
| HUMCEA_PEA_1_T30 (SEQ ID NO: 815) | 2093 | 2096 |

Segment cluster HUMCEA_PEA_{—}1_node_{—}62 (SEQ ID NO:861) according to the present invention is supported by 60 libraries. The number of libraries was determined as previously described. This segment can be found in the following transcript(s): HUMCEA_PEA_{—}1_T8 (SEQ ID NO:806), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T25 (SEQ ID NO:812), HUMCEA_PEA_{—}1_T26 (SEQ ID NO:813), HUMCEA_PEA_{—}1_T29 (SEQ ID NO:814) and HUMCEA_PEA_{—}1_T30 (SEQ ID NO:815). Table 74 below describes the starting and ending position of this segment on each transcript.

TABLE 74
Segment location on transcripts

| | Segment | Segment |
|---|---|---|
| | starting | ending |
| Transcript name | position | position |
| HUMCEA_PEA_1_T8 (SEQ ID NO: 806) | 2869 | 2956 |
| HUMCEA_PEA_1_T12 (SEQ ID NO: 808) | 2442 | 2529 |
| HUMCEA_PEA_1_T14 (SEQ ID NO: 809) | 3189 | 3276 |
| HUMCEA_PEA_1_T16 (SEQ ID NO: 810) | 1935 | 2022 |
| HUMCEA_PEA_1_T25 (SEQ ID NO: 812) | 1401 | 1488 |
| HUMCEA_PEA_1_T26 (SEQ ID NO: 813) | 1401 | 1488 |
| HUMCEA_PEA_1_T29 (SEQ ID NO: 814) | 844 | 931 |
| HUMCEA_PEA_1_T30 (SEQ ID NO: 815) | 2097 | 2184 |

Segment cluster HUMCEA_PEA_{—}1_node_{—}64 (SEQ ID NO:862) according to the present invention is supported by 45 libraries. The number of libraries was determined as previously described. This segment can be found in the following transcript(s): HUMCEA_PEA_{—}1_T8 (SEQ ID NO:806), HUMCEA_PEA_{—}1_T12 (SEQ ID NO:808), HUMCEA_PEA_{—}1_T14 (SEQ ID NO:809), HUMCEA_PEA_{—}1_T16 (SEQ ID NO:810), HUMCEA_PEA_{—}1_T25 (SEQ ID NO:812), HUMCEA_PEA_{—}1_T26 (SEQ ID NO:813), HUMCEA_PEA_{—}1_T29 (SEQ ID NO:814) and HUMCEA_PEA_{—}1_T30 (SEQ ID NO:815). Table 75 below describes the starting and ending position of this segment on each transcript.

TABLE 75
Segment location on transcripts

| | Segment | Segment |
|---|---|---|
| | starting | ending |
| Transcript name | position | position |
| HUMCEA_PEA_1_T8 (SEQ ID NO: 806) | 3136 | 3165 |
| HUMCEA_PEA_1_T12 (SEQ ID NO: 808) | 2709 | 2738 |
| HUMCEA_PEA_1_T14 (SEQ ID NO: 809) | 3456 | 3485 |
| HUMCEA_PEA_1_T16 (SEQ ID NO: 810) | 2202 | 2231 |
| HUMCEA_PEA_1_T25 (SEQ ID NO: 812) | 1668 | 1697 |
| HUMCEA_PEA_1_T26 (SEQ ID NO: 813) | 1668 | 1697 |
| HUMCEA_PEA_1_T29 (SEQ ID NO: 814) | 1111 | 1140 |
| HUMCEA_PEA_1_T30 (SEQ ID NO: 815) | 2364 | 2393 |

### Variant Protein Alignment to the Previously Known Protein:

Sequence name: CEA5_HUMAN (SEQ ID NO:863)
Sequence documentation:
Alignment of: HUMCEA_PEA_1_P4 (SEQ ID NO:864) × CEA5_HUMAN (SEQ ID NO:863)
Alignment segment 1/1:

| | | | |
|---|---|---|---|
| Quality: | 2320.00 | Escore | 0 |
| Matching length: | 234 | Total length: | 234 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

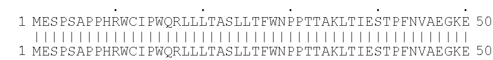

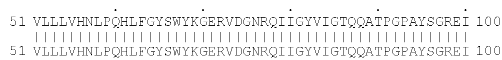

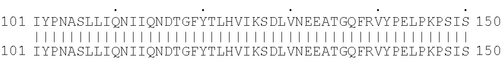

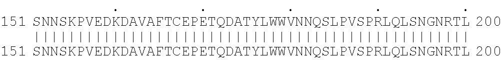

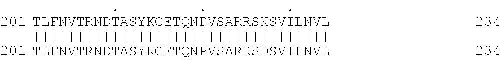

Sequence name: CEA5_HUMAN (SEQ ID NO:863)
Sequence documentation:
Alignment of: HUMCEA_PEA_1_P5 (SEQ ID NO:865) × CEA5_HUMAN (SEQ ID NO:863)
Alignment segment 1/1:

| | | | |
|---|---|---|---|
| Quality: | 6692.00 | Escore | 0 |
| Matching length: | 675 | Total length: | 675 |
| Matching Percent Similarity: | 100.00 | Matching Percent Identity: | 100.00 |
| Total Percent Similarity: | 100.00 | Total Percent Identity: | 100.00 |
| Caps: | 0 | | |
| Alignment: | | | |

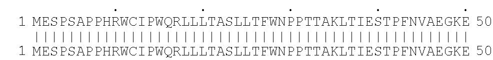

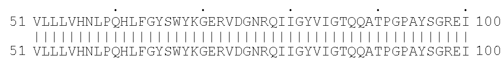

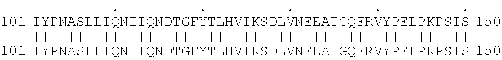

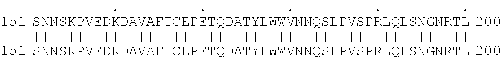

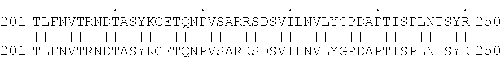

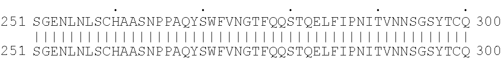

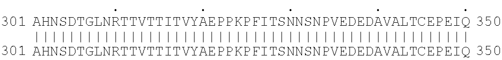

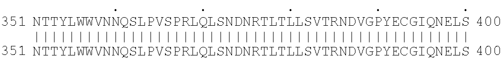

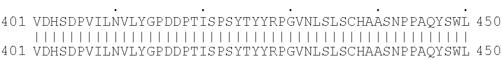

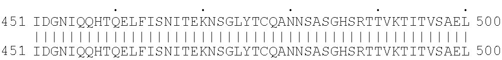

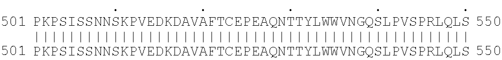

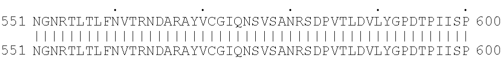

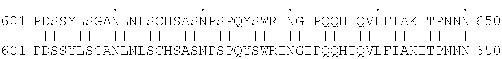

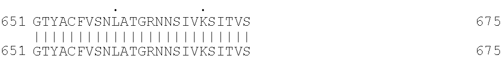

Sequence name: CEA5_HUMAN (SEQ ID NO:863)
Sequence documentation:
Alignment of: HUMCEA_PEA_1_P7 (SEQ ID NO:866) × CEA5_HUMAN (SEQ ID NO:863)
Alignment segment 1/1:

| | | | |
|---|---|---|---|
| Quality: | 6745.00 | Escore | 0 |
| Matching length: | 693 | Total length: | 702 |
| Matching Percent Similarity: | 100.00 | Matching Percent Identity: | 100.00 |
| Total Percent Similarity: | 98.72 | Total Percent Identity: | 98.72 |
| Gaps: | 1 | | |
| Alignment: | | | |

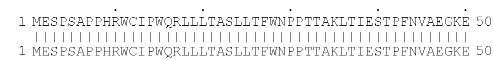

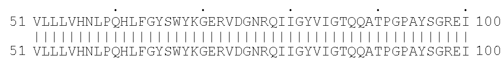

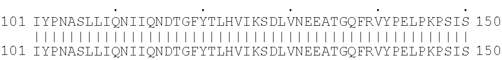

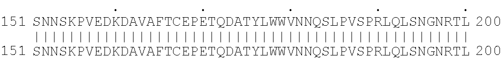

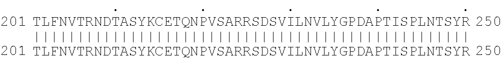

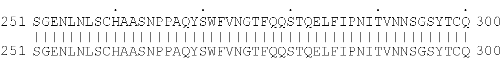

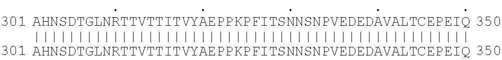

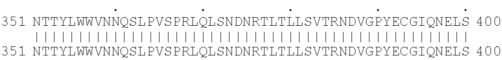

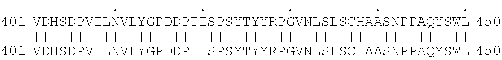

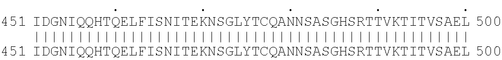

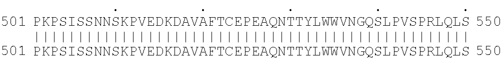

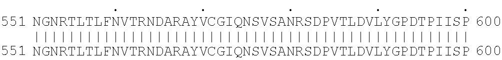

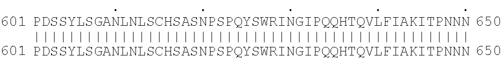

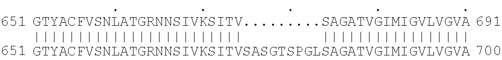

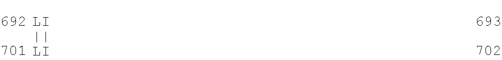

Sequence name: CEA5_HUMAN (SEQ ID NO:863)
Sequence documentation:
Alignment of: HUMCEA_PEA_1_P10 (SEQ ID NO:867) × CEA5_HUMAN (SEQ ID NO:863)
Alignment segment 1/1:

| | | | |
|---|---|---|---|
| Quality: | 5057.00 | Escore | 0 |
| Matching length: | 524 | Total length: | 702 |
| Matching Percent Similarity: | 100.00 | Matching Percent Identity: | 100.00 |
| Total Percent Similarity: | 74.64 | Total Percent Identity: | 74.64 |
| Gaps: | 1 | | |
| Alignment: | | | |

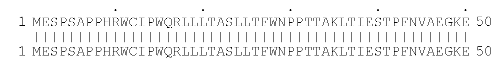

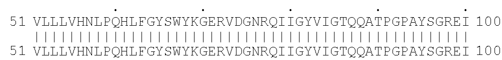

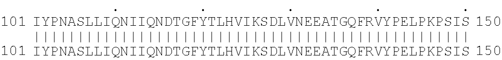

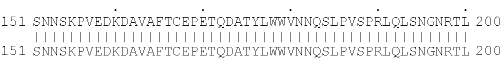

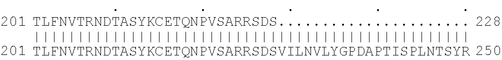

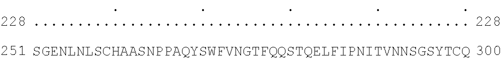

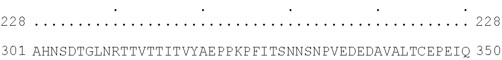

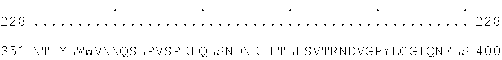

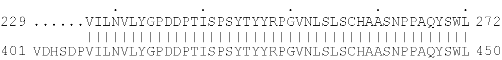

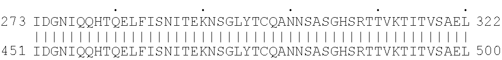

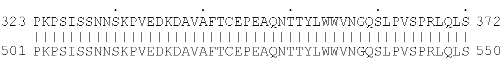

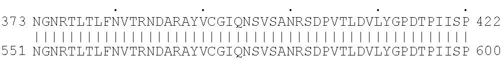

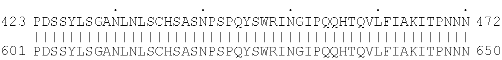

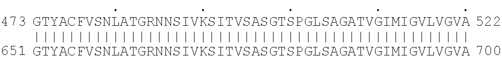

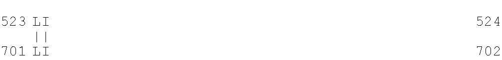

Sequence name: CEA5_HUMAN (SEQ ID NO:863)
Sequence documentation:
Alignment of: HUMCEA_PEA_1_P19 (SEQ ID NO:869) × CEA5_HUMAN (SEQ ID NO:863)
Alignment segment 1/1:

| | | | |
|---|---|---|---|
| Quality: | 3298.00 | Escore | 0 |
| Matching length: | 346 | Total length: | 702 |
| Matching Percent Similarity: | 100.00 | Matching Percent Identity: | 100.00 |
| Total Percent Similarity: | 49.29 | Total Percent Identity: | 49.29 |
| Gaps: | 1 | | |
| Alignment: | | | |

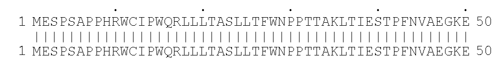

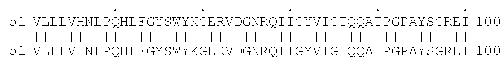

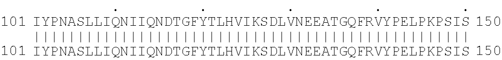

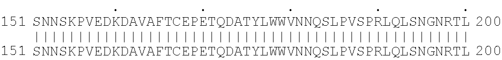

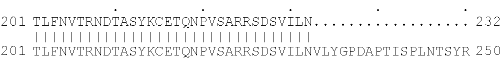

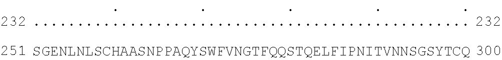

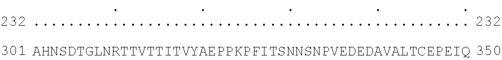

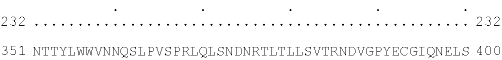

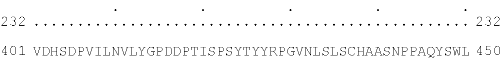

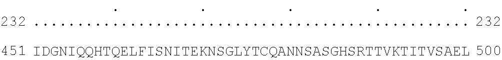

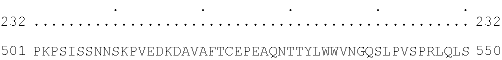

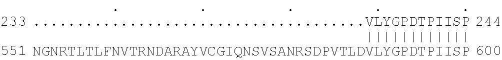

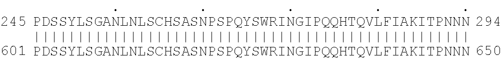

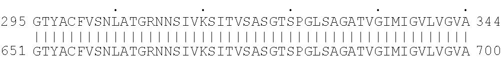

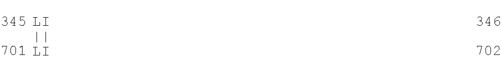

Sequence name: CEA5_HUMAN (SEQ ID NO:863)
Sequence documentation:
Alignment of: HUMCEA_PEA_1_P20 (SEQ ID NO:870) × CEA5_HUMAN (SEQ ID NO:863)
Alignment segment 1/1:

| | | | |
|---|---|---|---|
| Quality: | 3294.00 | Escore | 0 |
| Matching length: | 346 | Total length: | 702 |
| Matching Percent Similarity: | 100.00 | Matching Percent Identity: | 100.00 |
| Total Percent Similarity: | 49.29 | Total Percent Identity: | 49.29 |
| Gaps: | 1 | | |
| Alignment: | | | |

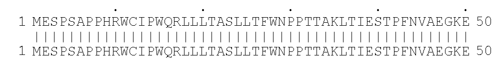

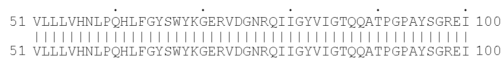

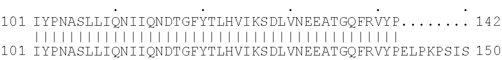

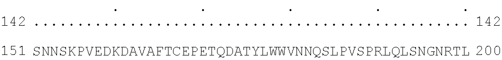

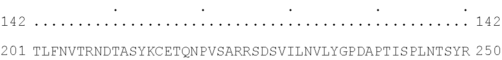

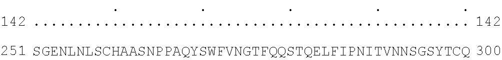

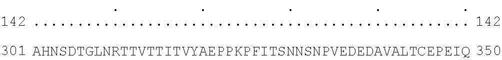

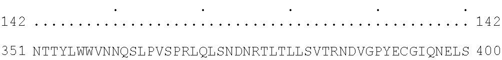

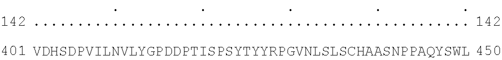

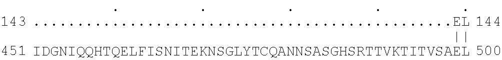

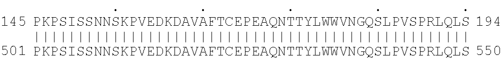

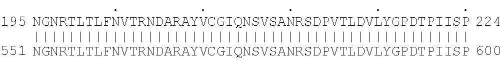

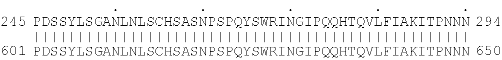

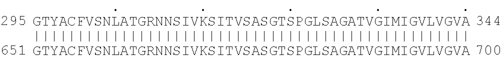

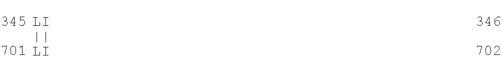

### Expression of Carcinoembryonic Antigen-Related Cell Adhesion Molecule 5 Transcripts which are Detectable by Seg12 and Seg9, in Normal, and Cancerous Colon Tissues

Expression of Carcinoembryonic antigen-related cell adhesion molecule 5 transcripts detectable by or according to seg12 (SEQ ID NO: 1338) and seg9 (SEQ ID NO: 1339), was measured with oligonucleotide-based micro-arrays. The results of image intensities for each feature were normalized according to the ninetieth percentile of the image intensities of all the features on the chip. Then, feature image intensities for replicates of the same oligonucleotide on the chip and replicates of the same sample were averaged. Outlying results were discarded.

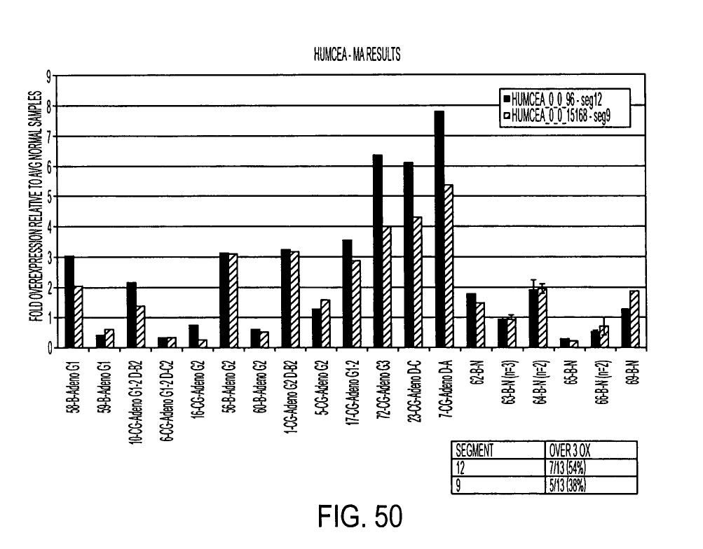

For every oligonucleotide HUMCEA_{—}0_{—}0_{—}96 (seg12, SEQ ID NO: 1338) and HUMCEA_{—}0_{—}0_{—}15168 (seg9, SEQ ID NO: 1339) the averaged intensity determined for every sample was divided by the averaged intensity of all the normal samples (Sample Nos. 62-66 and 69, Table 1, above, “Tissue samples in testing panel”), to obtain a value of fold up-regulation for each sample relative to the averaged normal samples. These data are presented in a histogram bellow, in FIG. 50. As is evident from the histogram (FIG. 50), the expression of Voltage-dependent L-type calcium channel alpha-1D subunit Calcium channel, L type, alpha-1 polypeptide, isoform 2 transcripts detectable with the above oligonucleotides in cancer samples was higher than in the normal samples.

HUMCEA_0_0_96-

(SEQ ID NO: 1338)

CAAGAGGGGTTTGGCTGAGACTTTAGGATTGTGATTCAGCTTAGAGGGAC
HUMCEA_0_0_15168-

(SEQ ID NO: 1339)

TCCTGCCTGTCACCTGAAGTTCTAGATCATTCCCTGGACTCCACTCTATC

### Expression of Carcinoembryonic Antigen-Related Cell Adhesion Molecule 5 CEACAM5 HUMCEA Transcripts which are Detectable by Amplicon as Depicted in Sequence Name HUMCEA Seg31 (SEQ ID NO: 1342) in Normal and Cancerous Colon Tissues

Expression of CEACAM5 transcripts detectable by or according to seg31, HUMCEA (ver 3.4 T10888) seg31 amplicon (SEQ ID NO: 1342) and HUMCEA seg31-F (SEQ ID NO: 1340) HUMCEA seg31-R (SEQ ID NO: 1341) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

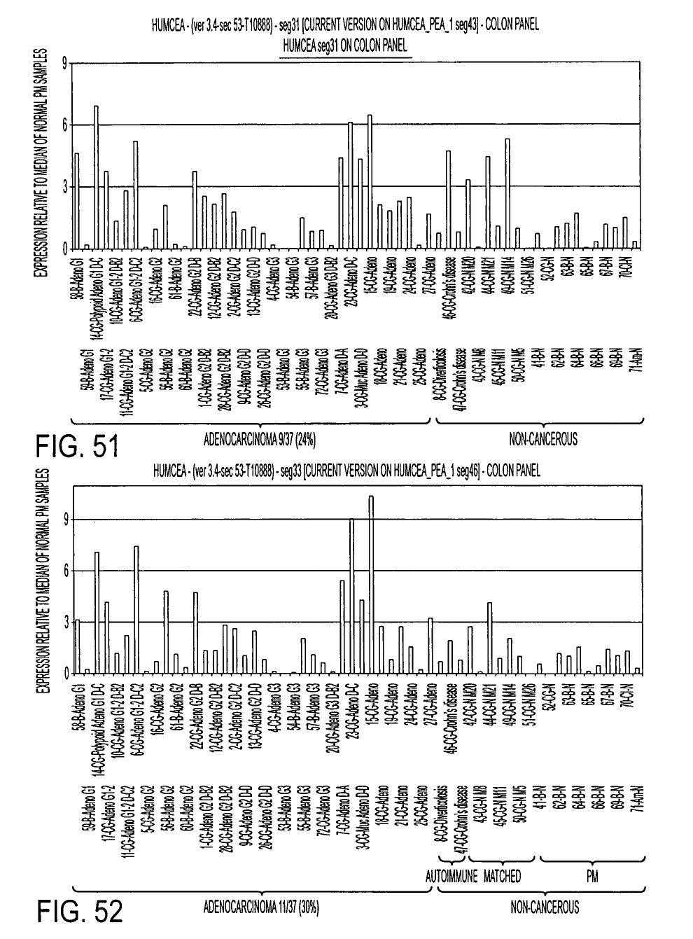

FIG. 51 is a histogram showing over expression of the above-indicated CEACAM5 transcripts in cancerous colon samples relative to the normal samples. The number and percentage of samples that exhibit at least 3 fold over-expression, out of the total number of samples tested is indicated in the bottom.

As is evident from FIG. 51, the expression of CEACAM5 transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 3 fold was found in 9 out of 37 adenocarcinoma samples.

Statistical analysis was applied to verify the significance of these results, as described below.

The P value for the difference in the expression levels of CEACAM5 transcripts detectable by the above amplicon in colon cancer samples versus the normal tissue samples was determined by T test as 6.24E-04. Threshold of 3 fold overexpression was found to differentiate between cancer and normal samples with P value of 7.42E-02 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: HUMCEA seg31F forward primer (SEQ ID NO: 1340); and HUMCEA seg31 R reverse primer (SEQ ID NO: 1341).

The present invention also preferably encompasses any amplicon obtained through the use of any suitable primer pair; for example, for the above experiment, the following amplicon was obtained as a non-limiting illustrative example only of a suitable amplicon: HUMCEA seg31 (SEQ ID NO: 1342).

Forward primer (SEQ ID NO: 1340):
CGGCCTCCCAAAGTGCT
Reverse primer (SEQ ID NO: 1341):
GGGAAGCTCCTGATTGTAGAAGG
Amplicon (SEQ ID NO: 1342):
CGGCCTCCCAAAGTGCTGGGATTACAGGCGTGAGCCACCGCACCCGGCCG
ATTTGGACTTTTTAACACAGGATTGGGACAGGATTCAGAGGGACACTGTG
GCCCTTCTACAATCAGGAGCTTCCC

### Expression of Carcinoembryonic Antigen-Related Cell Adhesion Molecule 5 CEACAM5 HUMCEA Transcripts which are Detectable by Amplicon as Depicted in Sequence Name HUMCEA Seg33 (SEQ ID NO: 1345) in Normal and Cancerous Colon Tissues

Expression of CEACAM5 transcripts detectable by or according to seg33, HUMCEA (ver 3.4 T10888) seg33 amplicon (SEQ ID NO: 1345) and HUMCEA seg33 F (SEQ ID NO: 1343) and HUMCEA seg33 R (SEQ ID NO: 1344) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

FIG. 52 is a histogram showing over expression of the above-indicated CEACAM5 transcripts in cancerous colon samples relative to the normal samples. The number and percentage of samples that exhibit at least 3 fold over-expression, out of the total number of samples tested is indicated in the bottom.

As is evident from FIG. 52, the expression of CEACAM5 transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 3 fold was found in 11 out of 37 adenocarcinoma samples,

Statistical analysis was applied to verify the significance of these results, as described below.

The P value for the difference in the expression levels of CEACAM5 transcripts detectable by the above amplicon in colon cancer samples versus the normal tissue samples was determined by T test as 4.01E-04. Threshold of 3 fold overexpression was found to differentiate between cancer and normal samples with P value of 3.78E-02 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: HUMCEA seg33F forward primer (SEQ ID NO: 1343); and HUMCEA seg33 R reverse primer (SEQ ID NO: 1344).

The present invention also preferably encompasses any amplicon obtained through the use of any suitable primer pair; for example, for the above experiment, the following amplicon was obtained as a non-limiting illustrative example only of a suitable amplicon: HUMCEA seg33 (SEQ ID NO: 1345).

Forward primer (SEQ ID NO: 1343):
CTGGAGCATCAGCATCATATTCTG
Reverse primer (SEQ ID NO: 1344):
GAGAGTTGGCCGAGATGGAG
Amplicon (SEQ ID NO: 1345):
CTGGAGCATCAGCATCATATTCTGGGGTGGAGTCTATCTGGTTCTCACCA
AAGAGCCAAGAAGACATTTTCTTTCCCAGTCTGTGTTCCATGGGCACAAG
GAAATCCCAAATTCTATCCTGAGCCCCCTCACTCCATCTCGGCCAACTCT
C

### Expression of Carcinoembryonic Antigen-Related Cell Adhesion Molecule 5 CEACAM5 HUMCEA Transcripts which are Detectable by Amplicon as Depicted in Sequence Name HUMCEA Seg35 (SEQ ID NO: 1348) in Normal and Cancerous Colon Tissues

Expression of CEACAM5 transcripts detectable by or according to seg35, HUMCEA (ver 3.4 T10888) seg35 amplicon (SEQ ID NO: 1348) and HUMCEA seg35 F (SEQ ID NO: 1346) and HUMCEA seg35 R (SEQ ID NO: 1347) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

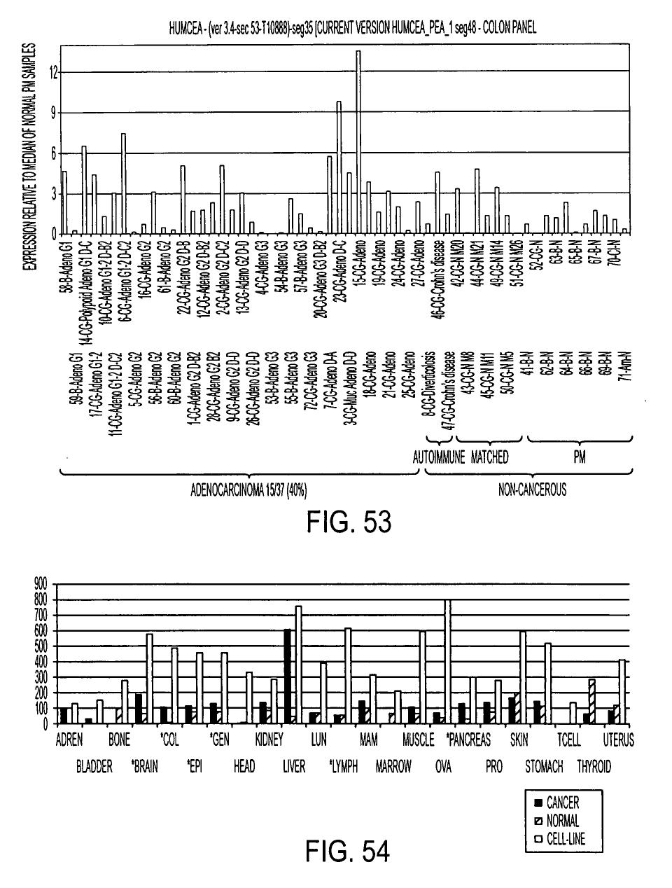

FIG. 53 is a histogram showing over expression of the above-indicated CEACAM5 transcripts in cancerous colon samples relative to the normal samples. The number and percentage of samples that exhibit at least 3 fold over-expression, out of the total number of samples tested is indicated in the bottom.

As is evident from FIG. 53, the expression of CEACAM5 transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 3 fold was found in 15 out of 37 adenocarcinoma samples,

Statistical analysis was applied to verify the significance of these results, as described below.

The P value for the difference in the expression levels of CEACAM5 transcripts detectable by the above amplicon in colon cancer samples versus the normal tissue samples was determined by T test as 8.96E-04. Threshold of 3 fold overexpression was found to differentiate between cancer and normal samples with P value of 1.27E-02 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: HUMCEA seg35F forward primer (SEQ ID NO: 1346); and HUMCEA seg35 R reverse primer (SEQ ID NO: 1347).

The present invention also preferably encompasses any amplicon obtained through the use of any suitable primer pair; for example, for the above experiment, the following amplicon was obtained as a non-limiting illustrative example only of a suitable amplicon: HUMCEA seg35 (SEQ ID NO: 1348).

Forward primer (SEQ ID NO: 1346):
GAAGCAGAGTCCCCCAGAACT
Reverse primer (SEQ ID NO: 1347):
AAGGCCCAGGCTAGTGCATT
Amplicon (SEQ ID NO: 1348):
GAAGCAGAGTCCCCCAGAACTGGGCTTTTCATTCCCCTGGTGGGAGCCCA
TGAGAAGCGAGTTCTCTGTGCAACGGACTTAGTAAATACAGAATGCACTA
GCCTGGGCCTT

### Description for Cluster M78035

Cluster M78035 features 12 transcript(s) and 39 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| M78035_T0 | 871 |
| M78035_T3 | 872 |
| M78035_T4 | 873 |
| M78035_T7 | 874 |
| M78035_T9 | 875 |
| M78035_T11 | 876 |
| M78035_T17 | 877 |
| M78035_T18 | 878 |
| M78035_T19 | 879 |
| M78035_T20 | 880 |
| M78035_T27 | 881 |
| M78035_T28 | 882 |

TABLE 2
Segments of interest

| Segment Name | SEQ ID NO: |
|---|---|
| M78035_node_4 | 883 |
| M78035_node_6 | 884 |
| M78035_node_10 | 885 |
| M78035_node_17 | 886 |
| M78035_node_18 | 887 |
| M78035_node_21 | 888 |
| M78035_node_25 | 889 |
| M78035_node_33 | 890 |
| M78035_node_55 | 891 |
| M78035_node_58 | 892 |
| M78035_node_60 | 893 |
| M78035_node_62 | 894 |
| M78035_node_63 | 895 |
| M78035_node_64 | 896 |
| M78035_node_65 | 897 |
| M78035_node_69 | 898 |
| M78035_node_71 | 899 |
| M78035_node_14 | 900 |
| M78035_node_15 | 901 |
| M78035_node_20 | 902 |
| M78035_node_24 | 903 |
| M78035_node_26 | 904 |
| M78035_node_28 | 905 |
| M78035_node_29 | 906 |
| M78035_node_30 | 907 |
| M78035_node_31 | 908 |
| M78035_node_34 | 909 |
| M78035_node_35 | 910 |
| M78035_node_37 | 911 |
| M78035_node_40 | 912 |
| M78035_node_48 | 913 |
| M78035_node_49 | 914 |
| M78035_node_50 | 915 |
| M78035_node_52 | 916 |
| M78035_node_53 | 917 |
| M78035_node_54 | 918 |
| M78035_node_56 | 919 |
| M78035_node_57 | 920 |
| M78035_node_59 | 921 |

TABLE 3
Proteins of interest

| Protein Name | SEQ ID NO: | Corresponding Transcript(s) |
|---|---|---|
| M78035_P2 | 923 | M78035_T0 (SEQ ID NO: 871); |
| | | M78035_T17 (SEQ ID NO: 877); |
| | | M78035_T18 (SEQ ID NO: 878); |
| | | M78035_T19 (SEQ ID NO: 879); |
| | | M78035_T20 (SEQ ID NO: 880) |
| M78035_P4 | 924 | M78035_T3 (SEQ ID NO: 872); |
| | | M78035_T4 (SEQ ID NO: 873) |
| M78035_P6 | 925 | M78035_T7 (SEQ ID NO: 874); |
| | | M78035_T9 (SEQ ID NO: 875) |
| M78035_P8 | 926 | M78035_T11 (SEQ ID NO: 876) |
| M78035_P18 | 927 | M78035_T27 (SEQ ID NO: 881) |
| M78035_P19 | 928 | M78035_T28 (SEQ ID NO: 882) |

These sequences are variants of the known protein Adenosylhomocysteinase (SwissProt accession identifier SAHH_HUMAN; known also according to the synonyms EC 3.3.1.1; S-adenosyl-L-homocysteine hydrolase; AdoHcyase), SEQ ID NO: 922, referred to herein as the previously known protein.

Protein Adenosylhomocysteinase (SEQ ID NO:922) is known or believed to have the following function(s): Adenosylhomocysteine is a competitive inhibitor of S-adenosyl-L-methionine-dependent methyl transferase reactions; therefore adenosylhomocysteinase may play a key role in the control of methylations via regulation of the intracellular concentration of adenosylhomocysteine. The sequence for protein Adenosylhomocysteinase is given at the end of the application, as “Adenosylhomocysteinase amino acid sequence”. Known polymorphisms for this sequence are as shown in Table 4.

TABLE 4
Amino acid mutations for Known Protein

| SNP position(s) on amino | |
|---|---|
| acid sequence | Comment |
| 86 | D -> N. /FTId = VAR_006934. |

Protein Adenosylhomocysteinase (SEQ ID NO:922) localization is believed to be Cytoplasmic.

The following GO Annotation(s) apply to the previously known protein. The following annotation(s) were found: one-carbon compound metabolism, which are annotation(s) related to Biological Process; adenosylhomocysteinase; hydrolase, which are annotation(s) related to Molecular Function; and cytoplasm, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster M78035 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 54 and Table 5. This cluster is overexpressed (at least at a minimum level) in the following pathological conditions: brain malignant tumors, colorectal cancer, epithelial malignant tumors, a mixture of malignant tumors from different tissues, malignant tumors involving the lymph nodes and pancreas carcinoma.

TABLE 5
Normal tissue distribution

| Name of Tissue | Number |
|---|---|
| adrenal | 0 |
| bladder | 0 |
| bone | 97 |
| brain | 71 |
| colon | 6 |
| epithelial | 74 |
| general | 72 |
| head and neck | 10 |
| kidney | 85 |
| liver | 48 |
| lung | 64 |
| lymph nodes | 49 |
| breast | 101 |
| bone marrow | 62 |
| muscle | 57 |
| ovary | 36 |
| pancreas | 30 |
| prostate | 76 |
| skin | 204 |
| stomach | 109 |
| T cells | 0 |
| Thyroid | 283 |
| uterus | 118 |

TABLE 6
P values and ratios for expression in cancerous tissue

| Name of Tissue | P1 | P2 | SP1 | R3 | SP2 | R4 |
|---|---|---|---|---|---|---|
| adrenal | 1.5e−01 | 7.0e−02 | 2.1e−01 | 3.4 | 1.5e−01 | 3.6 |
| bladder | 5.4e−01 | 3.4e−01 | 5.6e−01 | 1.8 | 2.1e−01 | 2.4 |
| bone | 9.2e−01 | 3.7e−02 | 1 | 0.2 | 2.1e−01 | 1.4 |
| brain | 1.5e−01 | 2.5e−02 | 3.8e−03 | 2.0 | 1.2e−12 | 3.7 |
| colon | 5.2e−02 | 2.3e−02 | 8.0e−02 | 3.7 | 2.0e−03 | 3.9 |
| epithelial | 3.1e−02 | 6.2e−05 | 1.7e−02 | 1.5 | 3.6e−25 | 3.4 |
| general | 6.7e−03 | 5.9e−10 | 3.8e−06 | 1.6 | 1.3e−66 | 3.8 |
| head and neck | 6.4e−01 | 2.5e−01 | 1 | 0.9 | 1.3e−01 | 2.1 |
| kidney | 7.6e−01 | 7.4e−01 | 2.5e−01 | 1.3 | 5.4e−02 | 1.7 |
| liver | 5.5e−02 | 1.1e−01 | 4.2e−02 | 5.1 | 4.4e−05 | 2.7 |
| lung | 8.0e−01 | 7.6e−01 | 6.4e−01 | 1.1 | 2.3e−04 | 2.6 |
| lymph nodes | 4.0e−01 | 1.6e−02 | 6.3e−01 | 1.1 | 2.5e−06 | 4.7 |
| breast | 3.4e−01 | 2.8e−01 | 4.3e−01 | 1.2 | 1.3e−01 | 1.3 |
| bone marrow | 7.5e−01 | 5.4e−01 | 1 | 0.3 | 1.9e−01 | 2.1 |
| muscle | 6.3e−01 | 5.0e−01 | 4.7e−01 | 1.6 | 7.1e−05 | 1.4 |
| ovary | 2.0e−01 | 1.1e−01 | 4.9e−01 | 1.5 | 3.5e−03 | 2.7 |
| pancreas | 4.5e−02 | 7.4e−03 | 2.3e−02 | 3.0 | 5.9e−04 | 3.2 |
| prostate | 4.7e−01 | 3.8e−01 | 1.4e−01 | 1.5 | 3.7e−02 | 1.6 |
| skin | 4.7e−01 | 5.3e−01 | 7.2e−01 | 0.8 | 6.9e−05 | 1.2 |
| stomach | 1.8e−01 | 1.7e−02 | 5.0e−01 | 1.2 | 6.2e−03 | 2.8 |
| T cells | 1 | 6.7e−01 | 1 | 1.0 | 7.2e−01 | 1.4 |
| Thyroid | 6.9e−01 | 6.9e−01 | 1 | 0.5 | 1 | 0.5 |
| uterus | 7.0e−01 | 3.4e−01 | 8.1e−01 | 0.7 | 5.6e−02 | 1.4 |

As noted above, cluster M178035 features 12 transcripts(s), which were listed in Table 1 above. These transcript(s) encode for protein(s) which are variant(s) of protein Adenosylhomocysteinase (SEQ ID NO:922). A description of each variant protein according to the present invention is now provided.

Variant protein M78035_P2 (SEQ ID NO:923) according to the present invention has an amino acid sequence as given at the end of the application; it is encoded by transcript(s) M78035_T0 (SEQ ID NO:871), M78035_T17 (SEQ ID NO:877), M78035_T18 (SEQ ID NO:878), M78035_T19 (SEQ ID NO:879) and M78035_T20 (SEQ ID NO:880). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein M78035_P2 (SEQ ID NO:923) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein M78035_P2 (SEQ ID NO:923) sequence provides support for the deduced sequence of this variant protein according to the present invention).

TABLE 7
Amino acid mutations

| SNP position(s) on | Alternative | Previously |
|---|---|---|
| amino acid sequence | amino acid(s) | known SNP? |
| 19 | R -> H | No |
| 36 | R -> L | No |
| 38 | R -> W | Yes |
| 65 | E -> D | No |
| 74 | V -> L | No |
| 77 | S -> | No |
| 91 | A -> | No |
| 111 | L -> P | No |
| 135 | L -> I | Yes |
| 206 | A -> | No |
| 206 | A -> G | No |
| 211 | I -> | No |
| 214 | K -> R | Yes |
| 226 | K -> | No |
| 226 | K -> Q | No |
| 265 | A -> | No |
| 325 | V -> L | No |
| 325 | V -> | No |
| 325 | V -> G | No |
| 353 | H -> Q | No |
| 353 | H -> | No |
| 374 | T -> | No |
| 383 | V -> G | No |
| 383 | V -> | No |
| 407 | T -> S | No |
| 407 | T -> | No |
| 414 | A -> T | No |

Variant protein M78035_P2 (SEQ ID NO:923) is encoded by the following transcript(s): M78035_T0 (SEQ ID NO:871), M78035_T17 (SEQ ID NO:877), M78035_T18 (SEQ ID NO:878), M78035_T19 (SEQ ID NO:879) and M78035_T20 (SEQ ID NO:880), for which the sequence(s) is/are given at the end of the application.

The coding portion of transcript M78035_T0 (SEQ ID NO:871) is shown in bold; this coding portion starts at position 132 and ends at position 1427. The transcript also has the following SNPs as listed in Table 8 (given according to their position on the nucleotide sequence, with the alternative nucleic acid listed; the last column indicates whether the SNP is known or not; the presence of known SNPs in variant protein M78035_P2 (SEQ ID NO:923) sequence provides support for the deduced sequence of this variant protein according to the present invention).

TABLE 8
Nucleic acid SNPs

| SNP position on | Alternative | Previously |
|---|---|---|
| nucleotide sequence | nucleic acid | known SNP? |
| 8 | C -> A | Yes |
| 140 | C -> T | No |
| 187 | G -> A | No |
| 197 | G -> A | No |
| 238 | G -> T | No |
| 243 | C -> T | Yes |
| 326 | G -> T | No |
| 335 | C -> T | No |
| 351 | G -> C | No |
| 361 | C -> | No |
| 403 | C -> | No |
| 463 | T -> C | No |
| 476 | G -> A | No |
| 482 | C -> A | No |
| 506 | C -> T | Yes |
| 534 | C -> A | Yes |
| 748 | C -> | No |
| 748 | C -> G | No |
| 763 | T -> | No |
| 772 | A -> G | Yes |
| 807 | A -> | No |
| 807 | A -> C | No |
| 925 | C -> | No |
| 1016 | T -> C | No |
| 1085 | G -> A | Yes |
| 1104 | G -> | No |
| 1104 | G -> T | No |
| 1105 | T -> G | No |
| 1190 | C -> | No |
| 1190 | C -> A | No |
| 1252 | C -> | No |
| 1279 | T -> | No |
| 1279 | T -> G | No |
| 1351 | C -> | No |
| 1351 | C -> G | No |
| 1371 | G -> A | No |
| 1505 | C -> | No |
| 1554 | C -> | No |
| 1554 | C -> G | No |
| 1563 | T -> C | No |
| 1573 | C -> | No |
| 1573 | C -> G | No |
| 1624 | C -> | No |
| 1624 | C -> A | No |
| 1638 | C -> G | No |
| 1664 | G -> | No |
| 1664 | G -> A | No |
| 1686 | T -> | No |
| 1712 | T -> C | No |
| 1719 | C -> | No |
| 1731 | T -> G | Yes |
| 1741 | A -> | No |
| 1741 | A -> C | No |
| 1755 | T -> | No |
| 1775 | C -> | No |
| 1775 | C -> A | No |
| 1807 | T -> | No |
| 1807 | T -> G | No |
| 1881 | T -> | No |
| 1881 | T -> G | No |
| 1937 | A -> | No |
| 1937 | A -> C | No |
| 1958 | C -> | No |
| 1969 | -> C | No |
| 1969 | -> G | No |
| 2001 | A -> C | Yes |
| 2008 | A -> C | Yes |
| 2009 | C -> G | Yes |
| 2011 | G -> C | Yes |
| 2016 | G -> T | Yes |
| 2041 | C -> T | No |
| 2043 | G -> T | No |
| 2047 | A -> C | Yes |
| 2084 | A -> C | Yes |
| 2086 | T -> | No |
| 2109 | A -> T | Yes |
| 2153 | A -> C | No |
| 2192 | T -> C | No |

The coding portion of transcript M78035_T17 (SEQ ID NO:877) is shown in bold; this coding portion starts at position 132 and ends at position 1427. The transcript also has the following SNPs as listed in Table 9 (given according to their position on the nucleotide sequence, with the alternative nucleic acid listed; the last column indicates whether the SNP is known or not; the presence of known SNPs in variant protein M78035_P2 (SEQ ID NO:923) sequence provides support for the deduced sequence of this variant protein according to the present invention).

TABLE 9
Nucleic acid SNPs

| SNP position on | Alternative | Previously |
|---|---|---|
| nucleotide sequence | nucleic acid | known SNP? |
| 8 | C -> A | Yes |
| 140 | C -> T | No |
| 187 | G -> A | No |
| 197 | G -> A | No |
| 238 | G -> T | No |
| 243 | C -> T | Yes |
| 326 | G -> T | No |
| 335 | C -> T | No |
| 351 | G -> C | No |
| 361 | C -> | No |
| 403 | C -> | No |
| 463 | T -> C | No |
| 476 | G -> A | No |
| 482 | C -> A | No |
| 506 | C -> T | Yes |
| 534 | C -> A | Yes |
| 748 | C -> | No |
| 748 | C -> G | No |
| 763 | T -> | No |
| 772 | A -> G | Yes |
| 807 | A -> | No |
| 807 | A -> C | No |
| 925 | C -> | No |
| 1016 | T -> C | No |
| 1085 | G -> A | Yes |
| 1104 | G -> | No |
| 1104 | G -> T | No |
| 1105 | T -> G | No |
| 1190 | C -> | No |
| 1190 | C -> A | No |
| 1252 | C -> | No |
| 1279 | T -> | No |
| 1279 | T -> G | No |
| 1351 | C -> | No |
| 1351 | C -> G | No |
| 1371 | G -> A | No |
| 1567 | C -> T | Yes |
| 1668 | G -> A | Yes |
| 2289 | G -> A | Yes |
| 2815 | T -> C | Yes |
| 3258 | G -> A | No |
| 3260 | G -> A | No |

The coding portion of transcript M78035_T18 (SEQ ID NO:878) is shown in bold; this coding portion starts at position 132 and ends at position 1427. The transcript also has the following SNPs as listed in Table 10 (given according to their position on the nucleotide sequence, with the alternative nucleic acid listed; the last column indicates whether the SNP is known or not; the presence of known SNPs in variant protein M78035_P2 (SEQ ID NO:923) sequence provides support for the deduced sequence of this variant protein according to the present invention).

TABLE 10
Nucleic acid SNPs

| SNP position | | |
|---|---|---|
| on nucleotide | Alternative | Previously |
| sequence | nucleic acid | known SNP? |
| 8 | C -> A | Yes |
| 140 | C -> T | No |
| 187 | G -> A | No |
| 197 | G -> A | No |
| 238 | G -> T | No |
| 243 | C -> T | Yes |
| 326 | G -> T | No |
| 335 | C -> T | No |
| 351 | G -> C | No |
| 361 | C -> | No |
| 403 | C -> | No |
| 463 | T -> C | No |
| 476 | G -> A | No |
| 482 | C -> A | No |
| 506 | C -> T | Yes |
| 534 | C -> A | Yes |
| 748 | C -> | No |
| 748 | C -> G | No |
| 763 | T -> | No |
| 772 | A -> G | Yes |
| 807 | A -> | No |
| 807 | A -> C | No |
| 925 | C -> | No |
| 1016 | T -> C | No |
| 1085 | G -> A | Yes |
| 1104 | G -> | No |
| 1104 | G -> T | No |
| 1105 | T -> G | No |
| 1190 | C -> | No |
| 1190 | C -> A | No |
| 1252 | C -> | No |
| 1279 | T -> | No |
| 1279 | T -> G | No |
| 1351 | C -> | No |
| 1351 | C -> G | No |
| 1371 | G -> A | No |
| 1707 | C -> T | Yes |
| 1808 | G -> A | Yes |
| 2429 | G -> A | Yes |
| 2955 | T -> C | Yes |
| 3398 | G -> A | No |
| 3400 | G -> A | No |

The coding portion of transcript M78035_T19 (SEQ ID NO:879) is shown in bold; this coding portion starts at position 132 and ends at position 1427. The transcript also has the following SNPs as listed in Table 11 (given according to their position on the nucleotide sequence, with the alternative nucleic acid listed; the last column indicates whether the SNP is known or not; the presence of known SNPs in variant protein M78035_P2 (SEQ ID NO:923) sequence provides support for the deduced sequence of this variant protein according to the present invention).

TABLE 11
Nucleic acid SNPs

| SNP position | | |
|---|---|---|
| on nucleotide | Alternative | Previously |
| sequence | nucleic acid | known SNP? |
| 8 | C -> A | Yes |
| 140 | C -> T | No |
| 187 | G -> A | No |
| 197 | G -> A | No |
| 238 | G -> T | No |
| 243 | C -> T | Yes |
| 326 | G -> T | No |
| 335 | C -> T | No |
| 351 | G -> C | No |
| 361 | C -> | No |
| 403 | C -> | No |
| 463 | T -> C | No |
| 476 | G -> A | No |
| 482 | C -> A | No |
| 506 | C -> T | Yes |
| 534 | C -> A | Yes |
| 748 | C -> | No |
| 748 | C -> G | No |
| 763 | T -> | No |
| 772 | A -> G | Yes |
| 807 | A -> | No |
| 807 | A -> C | No |
| 925 | C -> | No |
| 1016 | T -> C | No |
| 1085 | G -> A | Yes |
| 1104 | G -> | No |
| 1104 | G -> T | No |
| 1105 | T -> G | No |
| 1190 | C -> | No |
| 1190 | C -> A | No |
| 1252 | C -> | No |
| 1279 | T -> | No |
| 1279 | T -> G | No |
| 1351 | C -> | No |
| 1351 | C -> G | No |
| 1371 | G -> A | No |
| 1569 | A -> C | Yes |
| 2440 | G -> A | Yes |
| 3110 | G -> C | Yes |
| 3323 | C -> T | Yes |
| 3630 | G -> A | No |

The coding portion of transcript M78035_T20 (SEQ ID NO:880) is shown in bold; this coding portion starts at position 132 and ends at position 1427. The transcript also has the following SNPs as listed in Table 12 (given according to their position on the nucleotide sequence, with the alternative nucleic acid listed; the last column indicates whether the SNP is known or not; the presence of known SNPs in variant protein M78035_P2 (SEQ ID NO:923) sequence provides support for the deduced sequence of this variant protein according to the present invention).

TABLE 12
Nucleic acid SNPs

| SNP position | | |
|---|---|---|
| on nucleotide | Alternative | Previously |
| sequence | nucleic acid | known SNP? |
| 8 | C -> A | Yes |
| 140 | C -> T | No |
| 187 | G -> A | No |
| 197 | G -> A | No |
| 238 | G -> T | No |
| 243 | C -> T | Yes |
| 326 | G -> T | No |
| 335 | C -> T | No |
| 351 | G -> C | No |
| 361 | C -> | No |
| 403 | C -> | No |
| 463 | T -> C | No |
| 476 | G -> A | No |
| 482 | C -> A | No |
| 506 | C -> T | Yes |
| 534 | C -> A | Yes |
| 748 | C -> | No |
| 748 | C -> G | No |
| 763 | T -> | No |
| 772 | A -> G | Yes |
| 807 | A -> | No |
| 807 | A -> C | No |
| 925 | C -> | No |
| 1016 | T -> C | No |
| 1085 | G -> A | Yes |
| 1104 | G -> | No |
| 1104 | G -> T | No |
| 1105 | T -> G | No |
| 1190 | C -> | No |
| 1190 | C -> A | No |
| 1252 | C -> | No |
| 1279 | T -> | No |
| 1279 | T -> G | No |
| 1351 | C -> | No |
| 1351 | C -> G | No |
| 1371 | G -> A | No |
| 1569 | A -> C | Yes |
| 2440 | G -> A | Yes |
| 2649 | G -> C | Yes |
| 2862 | C -> T | Yes |
| 3169 | G -> A | No |

Variant protein M78035_P4 (SEQ ID NO:924) according to the present invention has an amino acid sequence as given at the end of the application; it is encoded by transcript(s) M78035_T3 (SEQ ID NO:872) and M78035_T4 (SEQ ID NO:873). An alignment is given to the known protein (Adenosylhomocysteinase (SEQ ID NO:922)) at the end of the application. One or more alignments to one or more previously published protein sequences are given at the end of the application. A brief description of the relationship of the variant protein according to the present invention to each such aligned protein is as follows:

Comparison Report Between M78035_P4 (SEQ ID NO:924) and SAHH_HUMAN (SEQ ID NO:922):

1. An isolated chimeric polypeptide encoding for M78035_P4 (SEQ ID NO:924), comprising a first amino acid sequence being at least 90% homologous to MPGLMRMRERYSASKPLKGARIAGCLHMTVETAVLIETLVTLGAEVQWSSCNIFSTQDHAAAAIAKAGIP VYAWKGETDEEYLWCIEQTLYFKDGPLNMILDDGGDLTNLIHTKYPQLLPGIRGISEETTTGVHNLYKMM ANGILKVPAINVNDSVTKSKFDNLYGCRESLIDGIKRATDVMIAGKVAVVAGYGDVGKGCAQALRGFGA RVIITEIDPINALQAAMEGYEVTTMDEACQEGNIFVTTTGCIDIILGRHFEQMKDDAIVCNIGHFDVEIDVK WLNENAVEKVNIKPQVDRYRLKNGRRIILLAEGRLVNLGCAMGHPSFVMSNSFTNQVMAQIELWTHPDK YPVGVHFLPKKLDEAVAEAHLGKLNVKLTKLTEKQAQYLGMSCDGPFKPDHYRY corresponding to amino acids 29-432 of SAHH_HUMAN (SEQ ID NO:922), which also corresponds to amino acids 1-404 of M78035_P4 (SEQ ID NO:924).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein M78035_P4 (SEQ ID NO:924) also has the following non-silent SNPs (Single Nucleotide Polymorphisms) as listed in Table 13, (given according to their position(s) on the amino acid sequence, with the alternative amino acid(s) listed; the last column indicates whether the SNP is known or not; the presence of known SNPs in variant protein M78035_P4 (SEQ ID NO:924) sequence provides support for the deduced sequence of this variant protein according to the present invention).

TABLE 13
Amino acid mutations

| SNP position(s) | | |
|---|---|---|
| on amino acid | Alternative | Previously |
| sequence | amino acid(s) | known SNP? |
| 8 | R -> L | No |
| 10 | R -> W | Yes |
| 37 | E -> D | No |
| 46 | V -> L | No |
| 49 | S -> | No |
| 63 | A -> | No |
| 83 | L -> P | No |
| 107 | L -> I | Yes |
| 178 | A -> | No |
| 178 | A -> G | No |
| 183 | I -> | No |
| 186 | K -> R | Yes |
| 198 | K -> | No |
| 198 | K -> Q | No |
| 237 | A -> | No |
| 297 | V -> G | No |
| 297 | V -> L | No |
| 297 | V -> | No |
| 325 | H -> Q | No |
| 325 | H -> | No |
| 346 | T -> | No |
| 355 | V -> | No |
| 355 | V -> G | No |
| 379 | T -> | No |
| 379 | T -> S | No |
| 386 | A -> T | No |

Variant protein M78035_P4 (SEQ ID NO:924) is encoded by the following transcript(s): M78035_T3 (SEQ ID NO:872) and M78035_T4 (SEQ ID NO:873), for which the sequence(s) is/are given at the end of the application.

The coding portion of transcript M78035_T3 (SEQ ID NO:872) is shown in bold; this coding portion starts at position 301 and ends at position 1512. The transcript also has the following SNPs as listed in Table 14 (given according to their position on the nucleotide sequence, with the alternative nucleic acid listed; the last column indicates whether the SNP is known or not; the presence of known SNPs in variant protein M78035_P4 (SEQ ID NO:924) sequence provides support for the deduced sequence of this variant protein according to the present invention).

TABLE 14
Nucleic acid SNPs

| SNP position | | |
|---|---|---|
| on nucleotide | Alternative | Previously |
| sequence | nucleic acid | known SNP? |
| 104 | A -> G | Yes |
| 272 | G -> A | No |
| 282 | G -> A | No |
| 323 | G -> T | No |
| 328 | C -> T | Yes |
| 411 | G -> T | No |
| 420 | C -> T | No |
| 436 | G -> C | No |
| 446 | C -> | No |
| 488 | C -> | No |
| 548 | T -> C | No |
| 561 | G -> A | No |
| 567 | C -> A | No |
| 591 | C -> T | Yes |
| 619 | C -> A | Yes |
| 833 | C -> | No |
| 833 | C -> G | No |
| 848 | T -> | No |
| 857 | A -> G | Yes |
| 892 | A -> | No |
| 892 | A -> C | No |
| 1010 | C -> | No |
| 1101 | T -> C | No |
| 1170 | G -> A | Yes |
| 1189 | G -> | No |
| 1189 | G -> T | No |
| 1190 | T -> G | No |
| 1275 | C -> | No |
| 1275 | C -> A | No |
| 1337 | C -> | No |
| 1364 | T -> | No |
| 1364 | T -> G | No |
| 1436 | C -> | No |
| 1436 | C -> G | No |
| 1456 | G -> A | No |
| 1590 | C -> | No |
| 1639 | C -> | No |
| 1639 | C -> G | No |
| 1648 | T -> C | No |
| 1658 | C -> | No |
| 1658 | C -> G | No |
| 1709 | C -> | No |
| 1709 | C -> A | No |
| 1723 | C -> G | No |
| 1749 | G -> | No |
| 1749 | G -> A | No |
| 1771 | T -> | No |
| 1797 | T -> C | No |
| 1804 | C -> | No |
| 1816 | T -> G | Yes |
| 1826 | A -> | No |
| 1826 | A -> C | No |
| 1840 | T -> | No |
| 1860 | C -> | No |
| 1860 | C -> A | No |
| 1892 | T -> | No |
| 1892 | T -> G | No |
| 1966 | T -> | No |
| 1966 | T -> G | No |
| 2022 | A -> | No |
| 2022 | A -> C | No |
| 2043 | C -> | No |
| 2054 | -> C | No |
| 2054 | -> G | No |
| 2086 | A -> C | Yes |
| 2093 | A -> C | Yes |
| 2094 | C -> G | Yes |
| 2096 | G -> C | Yes |
| 2101 | G -> T | Yes |
| 2126 | C -> T | No |
| 2128 | G -> T | No |
| 2132 | A -> C | Yes |
| 2169 | A -> C | Yes |
| 2171 | T -> | No |
| 2194 | A -> T | Yes |
| 2238 | A -> C | No |
| 2277 | T -> C | No |

The coding portion of transcript M78035_T4 (SEQ ID NO:873) is shown in bold; this coding portion starts at position 897 and ends at position 2108. The transcript also has the following SNPs as listed in Table 15 (given according to their position on the nucleotide sequence, with the alternative nucleic acid listed; the last column indicates whether the SNP is known or not; the presence of known SNPs in variant protein M78035_P4 (SEQ ID NO:924) sequence provides support for the deduced sequence of this variant protein according to the present invention).

TABLE 15
Nucleic acid SNPs

| SNP position | | |
|---|---|---|
| on nucleotide | Alternative | Previously |
| sequence | nucleic acid | known SNP? |
| 654 | G -> A | Yes |
| 868 | G -> A | No |
| 878 | G -> A | No |
| 919 | G -> T | No |
| 924 | C -> T | Yes |
| 1007 | G -> T | No |
| 1016 | C -> T | No |
| 1032 | G -> C | No |
| 1042 | C -> | No |
| 1084 | C -> | No |
| 1144 | T -> C | No |
| 1157 | G -> A | No |
| 1163 | C -> A | No |
| 1187 | C -> T | Yes |
| 1215 | C -> A | Yes |
| 1429 | C -> | No |
| 1429 | C -> G | No |
| 1444 | T -> | No |
| 1453 | A -> G | Yes |
| 1488 | A -> | No |
| 1488 | A -> C | No |
| 1606 | C -> | No |
| 1697 | T -> C | No |
| 1766 | G -> A | Yes |
| 1785 | G -> | No |
| 1785 | G -> T | No |
| 1786 | T -> G | No |
| 1871 | C -> | No |
| 1871 | C -> A | No |
| 1933 | C -> | No |
| 1960 | T -> | No |
| 1960 | T -> G | No |
| 2032 | C -> | No |
| 2032 | C -> G | No |
| 2052 | G -> A | No |
| 2186 | C -> | No |
| 2235 | C -> | No |
| 2235 | C -> G | No |
| 2244 | T -> C | No |
| 2254 | C -> | No |
| 2254 | C -> G | No |
| 2305 | C -> | No |
| 2305 | C -> A | No |
| 2319 | C -> G | No |
| 2345 | G -> | No |
| 2345 | G -> A | No |
| 2367 | T -> | No |
| 2393 | T -> C | No |
| 2400 | C -> | No |
| 2412 | T -> G | Yes |
| 2422 | A -> | No |
| 2422 | A -> C | No |
| 2436 | T -> | No |
| 2456 | C -> | No |
| 2456 | C -> A | No |
| 2488 | T -> | No |
| 2488 | T -> G | No |
| 2562 | T -> | No |
| 2562 | T -> G | No |
| 2618 | A -> | No |
| 2618 | A -> C | No |
| 2639 | C -> | No |
| 2650 | -> C | No |
| 2650 | -> G | No |
| 2682 | A -> C | Yes |
| 2689 | A -> C | Yes |
| 2690 | C -> G | Yes |
| 2692 | G -> C | Yes |
| 2697 | G -> T | Yes |
| 2722 | C -> T | No |
| 2724 | G -> T | No |
| 2728 | A -> C | Yes |
| 2765 | A -> C | Yes |
| 2767 | T -> | No |
| 2790 | A -> T | Yes |
| 2834 | A -> C | No |
| 2873 | T -> C | No |

Variant protein M78035_P6 (SEQ ID NO:925) according to the present invention has an amino acid sequence as given at the end of the application; it is encoded by transcript(s) M78035_T7 (SEQ ID NO:874) and M78035_T9 (SEQ ID NO:875). An alignment is given to the known protein (Adenosylhomocysteinase (SEQ ID NO:922)) at the end of the application. One or more alignments to one or more previously published protein sequences are given at the end of the application. A brief description of the relationship of the variant protein according to the present invention to each such aligned protein is as follows:

Comparison Report Between M78035_P6 (SEQ ID NO:925) and SAHH_HUMAN (SEQ ID NO:922):

1. An isolated chimeric polypeptide encoding for M78035_P6 (SEQ ID NO:925), comprising a first amino acid sequence being at least 90% homologous to MILDDGGDLTNLIHTKYPQLLPGIRGISEETTTGVHNLYKMMANGILKVPAINVNDSVTKSKFDNLYGCRE SLIDGIKRATDVMIAGKVAVVAGYGDVGKGCAQALRGFGARVIITEIDPINALQAAMEGYEVTTMDEACQ EGNIFVTTTGCIDIILGRHFEQMKDDAIVCNIGHFDVEIDVKWLNENAVEKVNIKPQVDRYRLKNGRRIILL AEGRLVNLGCAMGHPSFVMSNSFTNQVMAQIELWTHPDKYPVGVHFLPKKLDEAVAEAHLGKLNVKLT KLTEKQAQYLGMSCDGPFKPDHYRY corresponding to amino acids 127-432 of SAHH_HUMAN (SEQ ID NO:922), which also corresponds to amino acids 1-306 of M78035_P6 (SEQ ID NO:925).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein M78035_P6 (SEQ ID NO:925) also has the following non-silent SNPs (Single Nucleotide Polymorphisms) as listed in Table 16, (given according to their position(s) on the amino acid sequence, with the alternative amino acid(s) listed; the last column indicates whether the SNP is known or not; the presence of known SNPs in variant protein M78035_P6 (SEQ ID NO:925) sequence provides support for the deduced sequence of this variant protein according to the present invention).

TABLE 16
Amino acid mutations

| SNP position(s) | | |
|---|---|---|
| on amino acid | Alternative | Previously |
| sequence | amino acid(s) | known SNP? |
| 9 | L -> I | Yes |
| 80 | A -> G | No |
| 80 | A -> | No |
| 85 | I -> | No |
| 88 | K -> R | Yes |
| 100 | K -> | No |
| 100 | K -> Q | No |
| 139 | A -> | No |
| 199 | V -> | No |
| 199 | V -> L | No |
| 199 | V -> G | No |
| 227 | H -> | No |
| 227 | H -> Q | No |
| 248 | T -> | No |
| 257 | V -> G | No |
| 257 | V -> | No |
| 281 | T -> S | No |
| 281 | T -> | No |
| 288 | A -> T | No |

Variant protein M78035_P6 (SEQ ID NO:925) is encoded by the following transcript(s): M78035_T7 (SEQ ID NO:874) and M78035_T9 (SEQ ID NO:875), for which the sequence(s) is/are given at the end of the application.

The coding portion of transcript M78035_T7 (SEQ ID NO:874) is shown in bold; this coding portion starts at position 556 and ends at position 1473. The transcript also has the following SNPs as listed in Table 17 (given according to their position on the nucleotide sequence, with the alternative nucleic acid listed; the last column indicates whether the SNP is known or not; the presence of known SNPs in variant protein M78035_P6 (SEQ ID NO:925) sequence provides support for the deduced sequence of this variant protein according to the present invention).

TABLE 17
Nucleic acid SNPs

| SNP position | | |
|---|---|---|
| on nucleotide | Alternative | Previously |
| sequence | nucleic acid | known SNP? |
| 8 | C -> A | Yes |
| 140 | C -> T | No |
| 187 | G -> A | No |
| 197 | G -> A | No |
| 238 | G -> T | No |
| 243 | C -> T | Yes |
| 326 | G -> T | No |
| 335 | C -> T | No |
| 351 | G -> C | No |
| 361 | C -> | No |
| 403 | C -> | No |
| 509 | T -> C | No |
| 522 | G -> A | No |
| 528 | C -> A | No |
| 552 | C -> T | Yes |
| 580 | C -> A | Yes |
| 794 | C -> | No |
| 794 | C -> G | No |
| 809 | T -> | No |
| 818 | A -> G | Yes |
| 853 | A -> | No |
| 853 | A -> C | No |
| 971 | C -> | No |
| 1062 | T -> C | No |
| 1131 | G -> A | Yes |
| 1150 | G -> | No |
| 1150 | G -> T | No |
| 1151 | T -> G | No |
| 1236 | C -> | No |
| 1236 | C -> A | No |
| 1298 | C -> | No |
| 1325 | T -> | No |
| 1325 | T -> G | No |
| 1397 | C -> | No |
| 1397 | C -> G | No |
| 1417 | G -> A | No |
| 1551 | C -> | No |
| 1600 | C -> | No |
| 1600 | C -> G | No |
| 1609 | T -> C | No |
| 1619 | C -> | No |
| 1619 | C -> G | No |
| 1670 | C -> | No |
| 1670 | C -> A | No |
| 1684 | C -> G | No |
| 1710 | G -> | No |
| 1710 | G -> A | No |
| 1732 | T -> | No |
| 1758 | T -> C | No |
| 1765 | C -> | No |
| 1777 | T -> G | Yes |
| 1787 | A -> | No |
| 1787 | A -> C | No |
| 1801 | T -> | No |
| 1821 | C -> | No |
| 1821 | C -> A | No |
| 1853 | T -> | No |
| 1853 | T -> G | No |
| 1927 | T -> | No |
| 1927 | T -> G | No |
| 1983 | A -> | No |
| 1983 | A -> C | No |
| 2004 | C -> | No |
| 2015 | -> C | No |
| 2015 | -> G | No |
| 2047 | A -> C | Yes |
| 2054 | A -> C | Yes |
| 2055 | C -> G | Yes |
| 2057 | G -> C | Yes |
| 2062 | G -> T | Yes |
| 2087 | C -> T | No |
| 2089 | G -> T | No |
| 2093 | A -> C | Yes |
| 2130 | A -> C | Yes |
| 2132 | T -> | No |
| 2155 | A -> T | Yes |
| 2199 | A -> C | No |
| 2238 | T -> C | No |

The coding portion of transcript M78035_T9 (SEQ ID NO:875) is shown in bold; this coding portion starts at position 768 and ends at position 1685. The transcript also has the following SNPs as listed in Table 18 (given according to their position on the nucleotide sequence, with the alternative nucleic acid listed; the last column indicates whether the SNP is known or not; the presence of known SNPs in variant protein M78035_P6 (SEQ ID NO:925) sequence provides support for the deduced sequence of this variant protein according to the present invention).

TABLE 18
Nucleic acid SNPs

| SNP position | | |
|---|---|---|
| on nucleotide | Alternative | Previously |
| sequence | nucleic acid | known SNP? |
| 104 | A -> G | Yes |
| 272 | G -> A | No |
| 282 | G -> A | No |
| 323 | G -> T | No |
| 328 | C -> T | Yes |
| 411 | G -> T | No |
| 420 | C -> T | No |
| 609 | G -> C | No |
| 619 | C -> | No |
| 661 | C -> | No |
| 721 | T -> C | No |
| 734 | G -> A | No |
| 740 | C -> A | No |
| 764 | C -> T | Yes |
| 792 | C -> A | Yes |
| 1006 | C -> | No |
| 1006 | C -> G | No |
| 1021 | T -> | No |
| 1030 | A -> G | Yes |
| 1065 | A -> | No |
| 1065 | A -> C | No |
| 1183 | C -> | No |
| 1274 | T -> C | No |
| 1343 | G -> A | Yes |
| 1362 | G -> | No |
| 1362 | G -> T | No |
| 1363 | T -> G | No |
| 1448 | C -> | No |
| 1448 | C -> A | No |
| 1510 | C -> | No |
| 1537 | T -> | No |
| 1537 | T -> G | No |
| 1609 | C -> | No |
| 1609 | C -> G | No |
| 1629 | G -> A | No |
| 1763 | C -> | No |
| 1812 | C -> | No |
| 1812 | C -> G | No |
| 1821 | T -> C | No |
| 1831 | C -> | No |
| 1831 | C -> G | No |
| 1882 | C -> | No |
| 1882 | C -> A | No |
| 1896 | C -> G | No |
| 1922 | G -> | No |
| 1922 | G -> A | No |
| 1944 | T -> | No |
| 1970 | T -> C | No |
| 1977 | C -> | No |
| 1989 | T -> G | Yes |
| 1999 | A -> | No |
| 1999 | A -> C | No |
| 2013 | T -> | No |
| 2033 | C -> | No |
| 2033 | C -> A | No |
| 2065 | T -> | No |
| 2065 | T -> G | No |
| 2139 | T -> | No |
| 2139 | T -> G | No |
| 2195 | A -> | No |
| 2195 | A -> C | No |
| 2216 | C -> | No |
| 2227 | -> C | No |
| 2227 | -> G | No |
| 2259 | A -> C | Yes |
| 2266 | A -> C | Yes |
| 2267 | C -> G | Yes |
| 2269 | G -> C | Yes |
| 2274 | G -> T | Yes |
| 2299 | C -> T | No |
| 2301 | G -> T | No |
| 2305 | A -> C | Yes |
| 2342 | A -> C | Yes |
| 2344 | T -> | No |
| 2367 | A -> T | Yes |
| 2411 | A -> C | No |
| 2450 | T -> C | No |

Variant protein M78035_P8 (SEQ ID NO:926) according to the present invention has an amino acid sequence as given at the end of the application; it is encoded by transcript(s) M78035_T11 (SEQ ID NO:876). An alignment is given to the known protein (Adenosylhomocysteinase (SEQ ID NO:922)) at the end of the application. One or more alignments to one or more previously published protein sequences are given at the end of the application. A brief description of the relationship of the variant protein according to the present invention to each such aligned protein is as follows:

Comparison Report Between M78035_P8 (SEQ ID NO:926) and SAHH_HUMAN (SEQ ID NO:922):

1. An isolated chimeric polypeptide encoding for M78035_P8 (SEQ ID NO:926), comprising a first amino acid sequence being at least 70%, optionally at least 80%, preferably at least 85%, more preferably at least 90% and most preferably at least 95% homologous to a polypeptide having the sequence MSDKLPYKV (SEQ ID NO:1474) corresponding to amino acids 1-9 of M78035_P8 (SEQ ID NO:926), and a second amino acid sequence being at least 90% homologous to VYAWKGETDEEYLWCIEQTLYFKDGPLNMILDDGGDLTNLIHTKYPQLLPGIRGISEETTTGVHNLYKMM ANGILKVPAINVNDSVTKSKFDNLYGCRESLIDGIKRATDVMIAGKVAVVAGYGDVGKGCAQALRGFGA RVIITEIDPINALQAAMEGYEVTTMDEACQEGNIFVTTTGCIDIILGRHFEQMKDDAIVCNIGHFDVEIDVK WLNENAVEKVNIKPQVDRYRLKNGRRIILLAEGRLVNLGCAMGHPSFVMSNSFTNQVMAQIELWTHPDK YPVGVHFLPKKLDEAVAEAHLGKLNVKLTKLTEKQAQYLGMSCDGPFKPDHYRY corresponding to amino acids 99-432 of SAHH_HUMAN (SEQ ID NO:922), which also corresponds to amino acids 10-343 of M78035_P8 (SEQ ID NO:926), wherein said first amino acid sequence and second amino acid sequence are contiguous and in a sequential order.

2. An isolated polypeptide encoding for a head of M78035_P8 (SEQ ID NO:926), comprising a polypeptide being at least 70%, optionally at least about 80%, preferably at least about 85%, more preferably at least about 90% and most preferably at least about 95% homologous to the sequence MSDKLPYKV (SEQ ID NO:1474) of M78035_P8 (SEQ ID NO:926).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein M78035_P8 (SEQ ID NO:926) also has the following non-silent SNPs (Single Nucleotide Polymorphisms) as listed in Table 19, (given according to their position(s) on the amino acid sequence, with the alternative amino acid(s) listed; the last column indicates whether the SNP is known or not; the presence of known SNPs in variant protein M78035_P8 (SEQ ID NO:926) sequence provides support for the deduced sequence of this variant protein according to the present invention).

TABLE 19
Amino acid mutations

| SNP position(s) | | |
|---|---|---|
| on amino acid | Alternative | Previously |
| sequence | amino acid(s) | known SNP? |
| 22 | L -> P | No |
| 46 | L -> I | Yes |
| 117 | A -> | No |
| 117 | A -> G | No |
| 122 | I -> | No |
| 125 | K -> R | Yes |
| 137 | K -> | No |
| 137 | K -> Q | No |
| 176 | A -> | No |
| 236 | V -> G | No |
| 236 | V -> L | No |
| 236 | V -> | No |
| 264 | H -> Q | No |
| 264 | H -> | No |
| 285 | T -> | No |
| 294 | V -> | No |
| 294 | V -> G | No |
| 318 | T -> | No |
| 318 | T -> S | No |
| 325 | A -> T | No |

Variant protein M78035_P8 (SEQ ID NO:926) is encoded by the following transcript(s): M78035_T11 (SEQ ID NO:876), for which the sequence(s) is/are given at the end of the application. The coding portion of transcript M78035_T11 (SEQ ID NO:876) is shown in bold; this coding portion starts at position 132 and ends at position 1160. The transcript also has the following SNPs as listed in Table 20 (given according to their position on the nucleotide sequence, with the alternative nucleic acid listed; the last column indicates whether the SNP is known or not; the presence of known SNPs in variant protein M78035_P8 (SEQ ID NO:926) sequence provides support for the deduced sequence of this variant protein according to the present invention).

TABLE 20
Nucleic acid SNPs

| SNP position on nucleotide | | Previously |
|---|---|---|
| sequence | Alternative nucleic acid | known SNP? |
| 8 | C -> A | Yes |
| 140 | C -> T | No |
| 196 | T -> C | No |
| 209 | G -> A | No |
| 215 | C -> A | No |
| 239 | C -> T | Yes |
| 267 | C -> A | Yes |
| 481 | C -> | No |
| 481 | C -> G | No |
| 496 | T -> | No |
| 505 | A -> G | Yes |
| 540 | A -> | No |
| 540 | A -> C | No |
| 658 | C -> | No |
| 749 | T -> C | No |
| 818 | G -> A | Yes |
| 837 | G -> | No |
| 837 | G -> T | No |
| 838 | T -> G | No |
| 923 | C -> | No |
| 923 | C -> A | No |
| 985 | C -> | No |
| 1012 | T -> | No |
| 1012 | T -> G | No |
| 1084 | C -> | No |
| 1084 | C -> G | No |
| 1104 | G -> A | No |
| 1238 | C -> | No |
| 1287 | C -> | No |
| 1287 | C -> G | No |
| 1296 | T -> C | No |
| 1306 | C -> | No |
| 1306 | C -> G | No |
| 1357 | C -> | No |
| 1357 | C -> A | No |
| 1371 | C -> G | No |
| 1397 | G -> | No |
| 1397 | G -> A | No |
| 1419 | T -> | No |
| 1445 | T -> C | No |
| 1452 | C -> | No |
| 1464 | T -> G | Yes |
| 1474 | A -> | No |
| 1474 | A -> C | No |
| 1488 | T -> | No |
| 1508 | C -> | No |
| 1508 | C -> A | No |
| 1540 | T -> | No |
| 1540 | T -> G | No |
| 1614 | T -> | No |
| 1614 | T -> G | No |
| 1670 | A -> | No |
| 1670 | A -> C | No |
| 1691 | C -> | No |
| 1702 | -> C | No |
| 1702 | -> G | No |
| 1734 | A -> C | Yes |
| 1741 | A -> C | Yes |
| 1742 | C -> G | Yes |
| 1744 | G -> C | Yes |
| 1749 | G -> T | Yes |
| 1774 | C -> T | No |
| 1776 | G -> T | No |
| 1780 | A -> C | Yes |
| 1817 | A -> C | Yes |
| 1819 | T -> | No |
| 1842 | A -> T | Yes |
| 1886 | A -> C | No |
| 1925 | T -> C | No |

Variant protein M78035_P18 (SEQ ID NO:927) according to the present invention has an amino acid sequence as given at the end of the application; it is encoded by transcript(s) M78035_T27 (SEQ ID NO:881). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein M78035_P18 (SEQ ID NO:927) also has the following non-silent SNPs (Single Nucleotide Polymorphisms) as listed in Table 21, (given according to their position(s) on the amino acid sequence, with the alternative amino acid(s) listed; the last column indicates whether the SNP is known or not; the presence of known SNPs in variant protein M78035_P18 (SEQ ID NO:927) sequence provides support for the deduced sequence of this variant protein according to the present invention).

TABLE 21
Amino acid mutations

| SNP position(s) on amino acid | | Previously |
|---|---|---|
| sequence | Alternative amino acid(s) | known SNP? |
| 19 | R -> H | No |
| 36 | R -> L | No |
| 38 | R -> W | Yes |
| 65 | E -> D | No |
| 131 | W -> C | No |
| 135 | P -> | No |
| 149 | P -> | No |

Variant protein M78035_P18 (SEQ ID NO:927) is encoded by the following transcript(s): M78035_T27 (SEQ ID NO:881), for which the sequence(s) is/are given at the end of the application. The coding portion of transcript M78035_T27 (SEQ ID NO:881) is shown in bold; this coding portion starts at position 132 and ends at position 617. The transcript also has the following SNPs as listed in Table 22 (given according to their position on the nucleotide sequence, with the alternative nucleic acid listed; the last column indicates whether the SNP is known or not; the presence of known SNPs in variant protein M78035_P18 (SEQ ID NO:927) sequence provides support for the deduced sequence of this variant protein according to the present invention).

TABLE 22
Nucleic acid SNPs

| SNP position on nucleotide | | Previously |
|---|---|---|
| sequence | Alternative nucleic acid | known SNP? |
| 8 | C -> A | Yes |
| 140 | C -> T | No |
| 187 | G -> A | No |
| 197 | G -> A | No |
| 238 | G -> T | No |
| 243 | C -> T | Yes |
| 326 | G -> T | No |
| 335 | C -> T | No |
| 524 | G -> C | No |
| 534 | C -> | No |
| 576 | C -> | No |
| 980 | G -> A | Yes |

Variant protein M78035_P19 (SEQ ID NO:928) according to the present invention has an amino acid sequence as given at the end of the application; it is encoded by transcript(s) M78035_T28 (SEQ ID NO:882). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein M78035_P19 (SEQ ID NO:928) is encoded by the following transcript(s): M78035_T28 (SEQ ID NO:882), for which the sequence(s) is/are given at the end of the application. The coding portion of transcript M78035_T28 (SEQ ID NO:882) is shown in bold; this coding portion starts at position 585 and ends at position 902. The transcript also has the following SNPs as listed in Table 23 (given according to their position on the nucleotide sequence, with the alternative nucleic acid listed; the last column indicates whether the SNP is known or not; the presence of known SNPs in variant protein M78035_P19 (SEQ ID NO:928) sequence provides support for the deduced sequence of this variant protein according to the present invention).

TABLE 23
Nucleic acid SNPs

| SNP position on nucleotide | | Previously |
|---|---|---|
| sequence | Alternative nucleic acid | known SNP? |
| 483 | G -> A | Yes |
| 1153 | G -> C | Yes |
| 1366 | C -> T | Yes |
| 1673 | G -> A | No |

As noted above, cluster M78035 features 39 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster M78035_node_{—}4 (SEQ ID NO:883) according to the present invention is supported by 163 libraries. The number of libraries was determined as previously described. This segment can be found in the following transcript(s): M78035_T0 (SEQ ID NO:871), M78035_T7 (SEQ ID NO:874), M78035_T11 (SEQ ID NO:876), M78035_T17 (SEQ ID NO:877), M78035_T18 (SEQ ID NO:878), M78035_T19 (SEQ ID NO:879), M78035_T20 (SEQ ID NO:880) and M78035_T27 (SEQ ID NO:881). Table 24 below describes the starting and ending position of this segment on each transcript.

TABLE 24
Segment location on transcripts

| | Segment | Segment |
|---|---|---|
| Transcript name | starting position | ending position |
| M78035_T0 (SEQ ID NO: 871) | 1 | 159 |
| M78035_T7 (SEQ ID NO: 874) | 1 | 159 |
| M78035_T11 (SEQ ID NO: 876) | 1 | 159 |
| M78035_T17 (SEQ ID NO: 877) | 1 | 159 |
| M78035_T18 (SEQ ID NO: 878) | 1 | 159 |
| M78035_T19 (SEQ ID NO: 879) | 1 | 159 |
| M78035_T20 (SEQ ID NO: 880) | 1 | 159 |
| M78035_T27 (SEQ ID NO: 881) | 1 | 159 |

Segment cluster M78035_node_{—}6 (SEQ ID NO:884) according to the present invention is supported by 5 libraries. The number of libraries was determined as previously described. This segment can be found in the following transcript(s): M78035_T4 (SEQ ID NO:873). Table 25 below describes the starting and ending position of this segment on each transcript.

TABLE 25
Segment location on transcripts

| | Segment | Segment |
|---|---|---|
| Transcript name | starting position | ending position |
| M78035_T4 (SEQ ID NO: 873) | 1 | 840 |

Segment cluster M78035_node_{—}10 (SEQ ID NO:885) according to the present invention is supported by 3 libraries. The number of libraries was determined as previously described. This segment can be found in the following transcript(s): M78035_T3 (SEQ ID NO:872) and M78035_T9 (SEQ ID NO:875). Table 26 below describes the starting and ending position of this segment on each transcript.

TABLE 26
Segment location on transcripts

| | Segment | Segment |
|---|---|---|
| Transcript name | starting position | ending position |
| M78035_T3 (SEQ ID NO: 872) | 1 | 244 |
| M78035_T9 (SEQ ID NO: 875) | 1 | 244 |

Segment cluster M78035_node_{—}17 (SEQ ID NO:886) according to the present invention is supported by 189 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T17 (SEQ ID NO:877), M78035_T18 (SEQ ID NO:878), M78035_T19 (SEQ ID NO:879), M78035_T20 (SEQ ID NO:880) and M78035_T27 (SEQ ID NO:881). Table 27 below describes the starting and ending position of this segment on each transcript.

TABLE 27
Segment location on transcripts

| | Segment | Segment |
|---|---|---|
| Transcript name | starting position | ending position |
| M78035_T0 (SEQ ID NO: 871) | 160 | 350 |
| M78035_T3 (SEQ ID NO: 872) | 245 | 435 |
| M78035_T4 (SEQ ID NO: 873) | 841 | 1031 |
| M78035_T7 (SEQ ID NO: 874) | 160 | 350 |
| M78035_T9 (SEQ ID NO: 875) | 245 | 435 |
| M78035_T17 (SEQ ID NO: 877) | 160 | 350 |
| M78035_T18 (SEQ ID NO: 878) | 160 | 350 |
| M78035_T19 (SEQ ID NO: 879) | 160 | 350 |
| M78035_T20 (SEQ ID NO: 880) | 160 | 350 |
| M78035_T27 (SEQ ID NO: 881) | 160 | 350 |

Segment cluster M78035_node_{—}18 (SEQ ID NO:887) according to the present invention is supported by 2 libraries. The number of libraries was determined as previously described. This segment can be found in the following transcript(s): M78035_T9 (SEQ ID NO:875) and M78035_T27 (SEQ ID NO:881). Table 28 below describes the starting and ending position of this segment on each transcript.

TABLE 28
Segment location on transcripts

| | Segment | Segment |
|---|---|---|
| Transcript name | starting position | ending position |
| M78035_T9 (SEQ ID NO: 875) | 436 | 608 |
| M78035_T27 (SEQ ID NO: 881) | 351 | 523 |

Segment cluster M78035_node_{—}21 (SEQ ID NO:888) according to the present invention is supported by 3 libraries. The number of libraries was determined as previously described. This segment can be found in the following transcript(s): M78035_T27 (SEQ ID NO:881). Table 29 below describes the starting and ending position of this segment on each transcript.

TABLE 29
Segment location on transcripts

| | Segment | Segment |
|---|---|---|
| Transcript name | starting position | ending position |
| M78035_T27 (SEQ ID NO: 881) | 600 | 998 |

Segment cluster M78035_node_{—}25 (SEQ ID NO:889) according to the present invention is supported by 171 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 30 below describes the starting and ending position of this segment on each transcript.

TABLE 30
Segment location on transcripts

| | Segment | Segment |
|---|---|---|
| Transcript name | starting position | ending position |
| M78035_T0 (SEQ ID NO: 871) | 427 | 569 |
| M78035_T3 (SEQ ID NO: 872) | 512 | 654 |
| M78035_T4 (SEQ ID NO: 873) | 1108 | 1250 |
| M78035_T7 (SEQ ID NO: 874) | 473 | 615 |
| M78035_T9 (SEQ ID NO: 875) | 685 | 827 |
| M78035_T11 (SEQ ID NO: 876) | 160 | 302 |
| M78035_T17 (SEQ ID NO: 877) | 427 | 569 |
| M78035_T18 (SEQ ID NO: 878) | 427 | 569 |
| M78035_T19 (SEQ ID NO: 879) | 427 | 569 |
| M78035_T20 (SEQ ID NO: 880) | 427 | 569 |

Segment cluster M78035_node_{—}33 (SEQ ID NO:890) according to the present invention is supported by 191 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 31 below describes the starting and ending position of this segment on each transcript.

TABLE 31
Segment location on transcripts

| | Segment | Segment |
|---|---|---|
| Transcript name | starting position | ending position |
| M78035_T0 (SEQ ID NO: 871) | 690 | 860 |
| M78035_T3 (SEQ ID NO: 872) | 775 | 945 |
| M78035_T4 (SEQ ID NO: 873) | 1371 | 1541 |
| M78035_T7 (SEQ ID NO: 874) | 736 | 906 |
| M78035_T9 (SEQ ID NO: 875) | 948 | 1118 |
| M78035_T11 (SEQ ID NO: 876) | 423 | 593 |
| M78035_T17 (SEQ ID NO: 877) | 690 | 860 |
| M78035_T18 (SEQ ID NO: 878) | 690 | 860 |
| M78035_T19 (SEQ ID NO: 879) | 690 | 860 |
| M78035_T20 (SEQ ID NO: 880) | 690 | 860 |

Segment cluster M78035_node_{—}55 (SEQ ID NO:891) according to the present invention is supported by 238 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875) and M78035_T11 (SEQ ID NO:876). Table 32 below describes the starting and ending position of this segment on each transcript.

TABLE 32
Segment location on transcripts

| | Segment | Segment |
|---|---|---|
| Transcript name | starting position | ending position |
| M78035_T0 (SEQ ID NO: 871) | 1438 | 1578 |
| M78035_T3 (SEQ ID NO: 872) | 1523 | 1663 |
| M78035_T4 (SEQ ID NO: 873) | 2119 | 2259 |
| M78035_T7 (SEQ ID NO: 874) | 1484 | 1624 |
| M78035_T9 (SEQ ID NO: 875) | 1696 | 1836 |
| M78035_T11 (SEQ ID NO: 876) | 1171 | 1311 |

Segment cluster M78035_node_{—}58 (SEQ ID NO:892) according to the present invention is supported by 273 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875) and M78035_T11 (SEQ ID NO:876). Table 33 below describes the starting and ending position of this segment on each transcript.

TABLE 33
Segment location on transcripts

| | Segment | Segment |
|---|---|---|
| Transcript name | starting position | ending position |
| M78035_T0 (SEQ ID NO: 871) | 1663 | 1813 |
| M78035_T3 (SEQ ID NO: 872) | 1748 | 1898 |
| M78035_T4 (SEQ ID NO: 873) | 2344 | 2494 |
| M78035_T7 (SEQ ID NO: 874) | 1709 | 1859 |
| M78035_T9 (SEQ ID NO: 875) | 1921 | 2071 |
| M78035_T11 (SEQ ID NO: 876) | 1396 | 1546 |

Segment cluster M78035_node_{—}60 (SEQ ID NO:893) according to the present invention is supported by 268 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875) and M78035_T11 (SEQ ID NO:876). Table 34 below describes the starting and ending position of this segment on each transcript.

TABLE 34
Segment location on transcripts

| | Segment | Segment |
|---|---|---|
| Transcript name | starting position | ending position |
| M78035_T0 (SEQ ID NO: 871) | 1899 | 2194 |
| M78035_T3 (SEQ ID NO: 872) | 1984 | 2279 |
| M78035_T4 (SEQ ID NO: 873) | 2580 | 2875 |
| M78035_T7 (SEQ ID NO: 874) | 1945 | 2240 |
| M78035_T9 (SEQ ID NO: 875) | 2157 | 2452 |
| M78035_T11 (SEQ ID NO: 876) | 1632 | 1927 |

Segment cluster M78035_node_{—}62 (SEQ ID NO:894) according to the present invention is supported by 4 libraries. The number of libraries was determined as previously described. This segment can be found in the following transcript(s): M78035_T19 (SEQ ID NO:879) and M78035_T20 (SEQ ID NO:880). Table 35 below describes the starting and ending position of this segment on each transcript.

TABLE 35
Segment location on transcripts

| | Segment | Segment |
|---|---|---|
| Transcript name | starting position | ending position |
| M78035_T19 (SEQ ID NO: 879) | 1438 | 2099 |
| M78035_T20 (SEQ ID NO: 880) | 1438 | 2099 |

Segment cluster M78035_node_{—}63 (SEQ ID NO:895) according to the present invention is supported by 8 libraries. The number of libraries was determined as previously described. This segment can be found in the following transcript(s): M78035_T19 (SEQ ID NO:879), M78035_T20 (SEQ ID NO:880) and M78035_T28 (SEQ ID NO:882). Table 36 below describes the starting and ending position of this segment on each transcript.

TABLE 36
Segment location on transcripts

| | Segment | Segment |
|---|---|---|
| Transcript name | starting position | ending position |
| M78035_T19 (SEQ ID NO: 879) | 2100 | 2490 |
| M78035_T20 (SEQ ID NO: 880) | 2100 | 2490 |
| M78035_T28 (SEQ ID NO: 882) | 143 | 533 |

Segment cluster M78035_node_{—}64 (SEQ ID NO:896) according to the present invention is supported by 5 libraries. The number of libraries was determined as previously described. This segment can be found in the following transcript(s): M78035_T19 (SEQ ID NO:879) and M78035_T28 (SEQ ID NO:882). Table 37 below describes the starting and ending position of this segment on each transcript.

TABLE 37
Segment location on transcripts

| | Segment | Segment |
|---|---|---|
| Transcript name | starting position | ending position |
| M78035_T19 (SEQ ID NO: 879) | 2491 | 2951 |
| M78035_T28 (SEQ ID NO: 882) | 534 | 994 |

Segment cluster M78035_node_{—}65 (SEQ ID NO:897) according to the present invention is supported by 3 libraries. The number of libraries was determined as previously described. This segment can be found in the following transcript(s): M78035_T19 (SEQ ID NO:879), M78035_T20 (SEQ ID NO:880) and M78035_T28 (SEQ ID NO:882). Table 38 below describes the starting and ending position of this segment on each transcript.

TABLE 38
Segment location on transcripts

| | Segment | Segment |
|---|---|---|
| Transcript name | starting position | ending position |
| M78035_T19 (SEQ ID NO: 879) | 2952 | 3919 |
| M78035_T20 (SEQ ID NO: 880) | 2491 | 3458 |
| M78035_T28 (SEQ ID NO: 882) | 995 | 1962 |

Segment cluster M78035_node_{—}69 (SEQ ID NO:898) according to the present invention is supported by 1 libraries. The number of libraries was determined as previously described. This segment can be found in the following transcript(s): M78035_T18 (SEQ ID NO:878). Table 39 below describes the starting and ending position of this segment on each transcript.

TABLE 39
Segment location on transcripts

| | Segment | Segment |
|---|---|---|
| Transcript name | starting position | ending position |
| M78035_T18 (SEQ ID NO: 878) | 1438 | 1577 |

Segment cluster M78035_node_{—}71 (SEQ ID NO:899) according to the present invention is supported by 7 libraries. The number of libraries was determined as previously described. This segment can be found in the following transcript(s): M78035_T17 (SEQ ID NO:877) and M78035_T18 (SEQ ID NO:878). Table 40 below describes the starting and ending position of this segment on each transcript.

TABLE 40
Segment location on transcripts

| | Segment | Segment |
|---|---|---|
| Transcript name | starting position | ending position |
| M78035_T17 (SEQ ID NO: 877) | 1438 | 3302 |
| M78035_T18 (SEQ ID NO: 878) | 1578 | 3442 |

According to an optional embodiment of the present invention, short segments related to the above cluster are also provided. These segments are up to about 120 bp in length, and so are included in a separate description.

Segment cluster M78035_node_{—}14 (SEQ ID NO:900) according to the present invention is supported by 2 libraries. The number of libraries was determined as previously described. This segment can be found in the following transcript(s): M78035_T28 (SEQ ID NO:882). Table 41 below describes the starting and ending position of this segment on each transcript.

TABLE 41
Segment location on transcripts

| | Segment | Segment |
|---|---|---|
| Transcript name | starting position | ending position |
| M78035_T28 (SEQ ID NO: 882) | 1 | 95 |

Segment cluster M78035_node_{—}15 (SEQ ID NO:901) according to the present invention is supported by 1 libraries. The number of libraries was determined as previously described. This segment can be found in the following transcript(s): M78035_T28 (SEQ ID NO:882). Table 42 below describes the starting and ending position of this segment on each transcript.

TABLE 42
Segment location on transcripts

| | Segment | Segment |
|---|---|---|
| Transcript name | starting position | ending position |
| M78035_T28 (SEQ ID NO: 882) | 96 | 142 |

Segment cluster M78035_node_{—}20 (SEQ ID NO:902) according to the present invention is supported by 162 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T17 (SEQ ID NO:877), M78035_T18 (SEQ ID NO:878), M78035_T19 (SEQ ID NO:879), M78035_T20 (SEQ ID NO:880) and M78035_T27 (SEQ ID NO:881). Table 43 below describes the starting and ending position of this segment on each transcript.

TABLE 43
Segment location on transcripts

| | Segment | Segment |
|---|---|---|
| Transcript name | starting position | ending position |
| M78035_T0 (SEQ ID NO: 871) | 351 | 426 |
| M78035_T3 (SEQ ID NO: 872) | 436 | 511 |
| M78035_T4 (SEQ ID NO: 873) | 1032 | 1107 |
| M78035_T7 (SEQ ID NO: 874) | 351 | 426 |
| M78035_T9 (SEQ ID NO: 875) | 609 | 684 |
| M78035_T17 (SEQ ID NO: 877) | 351 | 426 |
| M78035_T18 (SEQ ID NO: 878) | 351 | 426 |
| M78035_T19 (SEQ ID NO: 879) | 351 | 426 |
| M78035_T20 (SEQ ID NO: 880) | 351 | 426 |
| M78035_T27 (SEQ ID NO: 881) | 524 | 599 |

Segment cluster M78035_node_{—}24 (SEQ ID NO:903) according to the present invention is supported by 1 libraries. The number of libraries was determined as previously described. This segment can be found in the following transcript(s): M78035_T7 (SEQ ID NO:874). Table 44 below describes the starting and ending position of this segment on each transcript.

TABLE 44
Segment location on transcripts

| | Segment | Segment |
|---|---|---|
| Transcript name | starting position | ending position |
| M78035_T7 (SEQ ID NO: 874) | 427 | 472 |

Segment cluster M78035_node_{—}26 (SEQ ID NO:904) according to the present invention can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 45 below describes the starting and ending position of this segment on each transcript.

TABLE 45
Segment location on transcripts

| | Segment | Segment |
|---|---|---|
| Transcript name | starting position | ending position |
| M78035_T0 (SEQ ID NO: 871) | 570 | 576 |
| M78035_T3 (SEQ ID NO: 872) | 655 | 661 |
| M78035_T4 (SEQ ID NO: 873) | 1251 | 1257 |
| M78035_T7 (SEQ ID NO: 874) | 616 | 622 |
| M78035_T9 (SEQ ID NO: 875) | 828 | 834 |
| M78035_T11 (SEQ ID NO: 876) | 303 | 309 |
| M78035_T17 (SEQ ID NO: 877) | 570 | 576 |
| M78035_T18 (SEQ ID NO: 878) | 570 | 576 |
| M78035_T19 (SEQ ID NO: 879) | 570 | 576 |
| M78035_T20 (SEQ ID NO: 880) | 570 | 576 |

Segment cluster M78035_node_{—}28 (SEQ ID NO:905) according to the present invention is supported by 161 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 46 below describes the starting and ending position of this segment on each transcript.

TABLE 46
Segment location on transcripts

| | Segment | Segment |
|---|---|---|
| Transcript name | starting position | ending position |
| M78035_T0 (SEQ ID NO: 871) | 577 | 622 |
| M78035_T3 (SEQ ID NO: 872) | 662 | 707 |
| M78035_T4 (SEQ ID NO: 873) | 1258 | 1303 |
| M78035_T7 (SEQ ID NO: 874) | 623 | 668 |
| M78035_T9 (SEQ ID NO: 875) | 835 | 880 |
| M78035_T11 (SEQ ID NO: 876) | 310 | 355 |
| M78035_T17 (SEQ ID NO: 877) | 577 | 622 |
| M78035_T18 (SEQ ID NO: 878) | 577 | 622 |
| M78035_T19 (SEQ ID NO: 879) | 577 | 622 |
| M78035_T20 (SEQ ID NO: 880) | 577 | 622 |

Segment cluster M78035_node_{—}29 (SEQ ID NO:906) according to the present invention is supported by 157 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 47 below describes the starting and ending position of this segment on each transcript.

TABLE 47
Segment location on transcripts

| | Segment | Segment |
|---|---|---|
| Transcript name | starting position | ending position |
| M78035_T0 (SEQ ID NO: 871) | 623 | 657 |
| M78035_T3 (SEQ ID NO: 872) | 708 | 742 |
| M78035_T4 (SEQ ID NO: 873) | 1304 | 1338 |
| M78035_T7 (SEQ ID NO: 874) | 669 | 703 |
| M78035_T9 (SEQ ID NO: 875) | 881 | 915 |
| M78035_T11 (SEQ ID NO: 876) | 356 | 390 |
| M78035_T17 (SEQ ID NO: 877) | 623 | 657 |
| M78035_T18 (SEQ ID NO: 878) | 623 | 657 |
| M78035_T19 (SEQ ID NO: 879) | 623 | 657 |
| M78035_T20 (SEQ ID NO: 880) | 623 | 657 |

Segment cluster M78035_node_{—}30 (SEQ ID NO:907) according to the present invention can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 48 below describes the starting and ending position of this segment on each transcript.

TABLE 48
Segment location on transcripts

| | Segment | Segment |
|---|---|---|
| Transcript name | starting position | ending position |
| M78035_T0 (SEQ ID NO: 871) | 658 | 664 |
| M78035_T3 (SEQ ID NO: 872) | 743 | 749 |
| M78035_T4 (SEQ ID NO: 873) | 1339 | 1345 |
| M78035_T7 (SEQ ID NO: 874) | 704 | 710 |
| M78035_T9 (SEQ ID NO: 875) | 916 | 922 |
| M78035_T11 (SEQ ID NO: 876) | 391 | 397 |
| M78035_T17 (SEQ ID NO: 877) | 658 | 664 |
| M78035_T18 (SEQ ID NO: 878) | 658 | 664 |
| M78035_T19 (SEQ ID NO: 879) | 658 | 664 |
| M78035_T20 (SEQ ID NO: 880) | 658 | 664 |

Segment cluster M78035_node_{—}31 (SEQ ID NO:908) according to the present invention can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 49 below describes the starting and ending position of this segment on each transcript.

TABLE 49
Segment location on transcripts

| | Segment | Segment |
|---|---|---|
| Transcript name | starting position | ending position |
| M78035_T0 (SEQ ID NO: 871) | 665 | 689 |
| M78035_T3 (SEQ ID NO: 872) | 750 | 774 |
| M78035_T4 (SEQ ID NO: 873) | 1346 | 1370 |
| M78035_T7 (SEQ ID NO: 874) | 711 | 735 |
| M78035_T9 (SEQ ID NO: 875) | 923 | 947 |
| M78035_T11 (SEQ ID NO: 876) | 398 | 422 |
| M78035_T17 (SEQ ID NO: 877) | 665 | 689 |
| M78035_T18 (SEQ ID NO: 878) | 665 | 689 |
| M78035_T19 (SEQ ID NO: 879) | 665 | 689 |
| M78035_T20 (SEQ ID NO: 880) | 665 | 689 |

Segment cluster M78035_node_{—}34 (SEQ ID NO:909) according to the present invention can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 50 below describes the starting and ending position of this segment on each transcript.

TABLE 50
Segment location on transcripts

| | Segment | Segment |
|---|---|---|
| Transcript name | starting position | ending position |
| M78035_T0 (SEQ ID NO: 871) | 861 | 878 |
| M78035_T3 (SEQ ID NO: 872) | 946 | 963 |
| M78035_T4 (SEQ ID NO: 873) | 1542 | 1559 |
| M78035_T7 (SEQ ID NO: 874) | 907 | 924 |
| M78035_T9 (SEQ ID NO: 875) | 1119 | 1136 |
| M78035_T11 (SEQ ID NO: 876) | 594 | 611 |
| M78035_T17 (SEQ ID NO: 877) | 861 | 878 |
| M78035_T18 (SEQ ID NO: 878) | 861 | 878 |
| M78035_T19 (SEQ ID NO: 879) | 861 | 878 |
| M78035_T20 (SEQ ID NO: 880) | 861 | 878 |

Segment cluster M78035_node_{—}35 (SEQ ID NO:910) according to the present invention can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 51 below describes the starting and ending position of this segment on each transcript.

TABLE 51
Segment location on transcripts

| | Segment | Segment |
|---|---|---|
| Transcript name | starting position | ending position |
| M78035_T0 (SEQ ID NO: 871) | 879 | 897 |
| M78035_T3 (SEQ ID NO: 872) | 964 | 982 |
| M78035_T4 (SEQ ID NO: 873) | 1560 | 1578 |
| M78035_T7 (SEQ ID NO: 874) | 925 | 943 |
| M78035_T9 (SEQ ID NO: 875) | 1137 | 1155 |
| M78035_T11 (SEQ ID NO: 876) | 612 | 630 |
| M78035_T17 (SEQ ID NO: 877) | 879 | 897 |
| M78035_T18 (SEQ ID NO: 878) | 879 | 897 |
| M78035_T19 (SEQ ID NO: 879) | 879 | 897 |
| M78035_T20 (SEQ ID NO: 880) | 879 | 897 |

Segment cluster M78035_node_{—}37 (SEQ ID NO:911) according to the present invention is supported by 177 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 52 below describes the starting and ending position of this segment on each transcript.

TABLE 52
Segment location on transcripts

| | Segment | Segment |
|---|---|---|
| Transcript name | starting position | ending position |
| M78035_T0 (SEQ ID NO: 871) | 898 | 985 |
| M78035_T3 (SEQ ID NO: 872) | 983 | 1070 |
| M78035_T4 (SEQ ID NO: 873) | 1579 | 1666 |
| M78035_T7 (SEQ ID NO: 874) | 944 | 1031 |
| M78035_T9 (SEQ ID NO: 875) | 1156 | 1243 |
| M78035_T11 (SEQ ID NO: 876) | 631 | 718 |
| M78035_T17 (SEQ ID NO: 877) | 898 | 985 |
| M78035_T18 (SEQ ID NO: 878) | 898 | 985 |
| M78035_T19 (SEQ ID NO: 879) | 898 | 985 |
| M78035_T20 (SEQ ID NO: 880) | 898 | 985 |

Segment cluster M78035_node_{—}40 (SEQ ID NO:912) according to the present invention is supported by 194 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 53 below describes the starting and ending position of this segment on each transcript.

TABLE 53
Segment location on transcripts

| | Segment | Segment |
|---|---|---|
| Transcript name | starting position | ending position |
| M78035_T0 (SEQ ID NO: 871) | 986 | 1103 |
| M78035_T3 (SEQ ID NO: 872) | 1071 | 1188 |
| M78035_T4 (SEQ ID NO: 873) | 1667 | 1784 |
| M78035_T7 (SEQ ID NO: 874) | 1032 | 1149 |
| M78035_T9 (SEQ ID NO: 875) | 1244 | 1361 |
| M78035_T11 (SEQ ID NO: 876) | 719 | 836 |
| M78035_T17 (SEQ ID NO: 877) | 986 | 1103 |
| M78035_T18 (SEQ ID NO: 878) | 986 | 1103 |
| M78035_T19 (SEQ ID NO: 879) | 986 | 1103 |
| M78035_T20 (SEQ ID NO: 880) | 986 | 1103 |

Segment cluster M78035_node_{—}48 (SEQ ID NO:913) according to the present invention is supported by 180 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 54 below describes the starting and ending position of this segment on each transcript.

TABLE 54
Segment location on transcripts

| | Segment | Segment |
|---|---|---|
| Transcript name | starting position | ending position |
| M78035_T0 (SEQ ID NO: 871) | 1104 | 1145 |
| M78035_T3 (SEQ ID NO: 872) | 1189 | 1230 |
| M78035_T4 (SEQ ID NO: 873) | 1785 | 1826 |
| M78035_T7 (SEQ ID NO: 874) | 1150 | 1191 |
| M78035_T9 (SEQ ID NO: 875) | 1362 | 1403 |
| M78035_T11 (SEQ ID NO: 876) | 837 | 878 |
| M78035_T17 (SEQ ID NO: 877) | 1104 | 1145 |
| M78035_T18 (SEQ ID NO: 878) | 1104 | 1145 |
| M78035_T19 (SEQ ID NO: 879) | 1104 | 1145 |
| M78035_T20 (SEQ ID NO: 880) | 1104 | 1145 |

Segment cluster M78035_node_{—}49 (SEQ ID NO:914) according to the present invention is supported by 190 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 55 below describes the starting and ending position of this segment on each transcript.

TABLE 55
Segment location on transcripts

| | Segment | Segment |
|---|---|---|
| Transcript name | starting position | ending position |
| M78035_T0 (SEQ ID NO: 871) | 1146 | 1226 |
| M78035_T3 (SEQ ID NO: 872) | 1231 | 1311 |
| M78035_T4 (SEQ ID NO: 873) | 1827 | 1907 |
| M78035_T7 (SEQ ID NO: 874) | 1192 | 1272 |
| M78035_T9 (SEQ ID NO: 875) | 1404 | 1484 |
| M78035_T11 (SEQ ID NO: 876) | 879 | 959 |
| M78035_T17 (SEQ ID NO: 877) | 1146 | 1226 |
| M78035_T18 (SEQ ID NO: 878) | 1146 | 1226 |
| M78035_T19 (SEQ ID NO: 879) | 1146 | 1226 |
| M78035_T20 (SEQ ID NO: 880) | 1146 | 1226 |

Segment cluster M78035_node_{—}50 (SEQ ID NO:915) according to the present invention is supported by 190 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 56 below describes the starting and ending position of this segment on each transcript.

TABLE 56
Segment location on transcripts

| | Segment | Segment |
|---|---|---|
| Transcript name | starting position | ending position |
| M78035_T0 (SEQ ID NO: 871) | 1227 | 1298 |
| M78035_T3 (SEQ ID NO: 872) | 1312 | 1383 |
| M78035_T4 (SEQ ID NO: 873) | 1908 | 1979 |
| M78035_T7 (SEQ ID NO: 874) | 1273 | 1344 |
| M78035_T9 (SEQ ID NO: 875) | 1485 | 1556 |
| M78035_T11 (SEQ ID NO: 876) | 960 | 1031 |
| M78035_T17 (SEQ ID NO: 877) | 1227 | 1298 |
| M78035_T18 (SEQ ID NO: 878) | 1227 | 1298 |
| M78035_T19 (SEQ ID NO: 879) | 1227 | 1298 |
| M78035_T20 (SEQ ID NO: 880) | 1227 | 1298 |

Segment cluster M78035_node_{—}52 (SEQ ID NO:916) according to the present invention can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 57 below describes the starting and ending position of this segment on each transcript.

TABLE 57
Segment location on transcripts

| | Segment | Segment |
|---|---|---|
| Transcript name | starting position | ending position |
| M78035_T0 (SEQ ID NO: 871) | 1299 | 1314 |
| M78035_T3 (SEQ ID NO: 872) | 1384 | 1399 |
| M78035_T4 (SEQ ID NO: 873) | 1980 | 1995 |
| M78035_T7 (SEQ ID NO: 874) | 1345 | 1360 |
| M78035_T9 (SEQ ID NO: 875) | 1557 | 1572 |
| M78035_T11 (SEQ ID NO: 876) | 1032 | 1047 |
| M78035_T17 (SEQ ID NO: 877) | 1299 | 1314 |
| M78035_T18 (SEQ ID NO: 878) | 1299 | 1314 |
| M78035_T19 (SEQ ID NO: 879) | 1299 | 1314 |
| M78035_T20 (SEQ ID NO: 880) | 1299 | 1314 |

Segment cluster M78035_node_{—}53 (SEQ ID NO:917) according to the present invention can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 58 below describes the starting and ending position of this segment on each transcript.

TABLE 58
Segment location on transcripts

| | Segment | Segment |
|---|---|---|
| Transcript name | starting position | ending position |
| M78035_T0 (SEQ ID NO: 871) | 1315 | 1334 |
| M78035_T3 (SEQ ID NO: 872) | 1400 | 1419 |
| M78035_T4 (SEQ ID NO: 873) | 1996 | 2015 |
| M78035_T7 (SEQ ID NO: 874) | 1361 | 1380 |
| M78035_T9 (SEQ ID NO: 875) | 1573 | 1592 |
| M78035_T11 (SEQ ID NO: 876) | 1048 | 1067 |
| M78035_T17 (SEQ ID NO: 877) | 1315 | 1334 |
| M78035_T18 (SEQ ID NO: 878) | 1315 | 1334 |
| M78035_T19 (SEQ ID NO: 879) | 1315 | 1334 |
| M78035_T20 (SEQ ID NO: 880) | 1315 | 1334 |

Segment cluster M78035_node_{—}54 (SEQ ID NO:918) according to the present invention is supported by 213 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875), M78035_T11 (SEQ ID NO:876), M78035_T17 (SEQ ID NO:877), M78035_T18 (SEQ ID NO:878), M78035_T19 (SEQ ID NO:879) and M78035_T20 (SEQ ID NO:880). Table 59 below describes the starting and ending position of this segment on each transcript.

TABLE 59
Segment location on transcripts

| | Segment | Segment |
|---|---|---|
| Transcript name | starting position | ending position |
| M78035_T0 (SEQ ID NO: 871) | 1335 | 1437 |
| M78035_T3 (SEQ ID NO: 872) | 1420 | 1522 |
| M78035_T4 (SEQ ID NO: 873) | 2016 | 2118 |
| M78035_T7 (SEQ ID NO: 874) | 1381 | 1483 |
| M78035_T9 (SEQ ID NO: 875) | 1593 | 1695 |
| M78035_T11 (SEQ ID NO: 876) | 1068 | 1170 |
| M78035_T17 (SEQ ID NO: 877) | 1335 | 1437 |
| M78035_T18 (SEQ ID NO: 878) | 1335 | 1437 |
| M78035_T19 (SEQ ID NO: 879) | 1335 | 1437 |
| M78035_T20 (SEQ ID NO: 880) | 1335 | 1437 |

Segment cluster M78035_node_{—}56 (SEQ ID NO:919) according to the present invention can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875) and M78035_T11 (SEQ ID NO:876). Table 60 below describes the starting and ending position of this segment on each transcript.

TABLE 60
Segment location on transcripts

| | Segment | Segment |
|---|---|---|
| Transcript name | starting position | ending position |
| M78035_T0 (SEQ ID NO: 871) | 1579 | 1592 |
| M78035_T3 (SEQ ID NO: 872) | 1664 | 1677 |
| M78035_T4 (SEQ ID NO: 873) | 2260 | 2273 |
| M78035_T7 (SEQ ID NO: 874) | 1625 | 1638 |
| M78035_T9 (SEQ ID NO: 875) | 1837 | 1850 |
| M78035_T11 (SEQ ID NO: 876) | 1312 | 1325 |

Segment cluster M78035_node_{—}57 (SEQ ID NO:920) according to the present invention is supported by 225 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875) and M78035_T11 (SEQ ID NO:876). Table 61 below describes the starting and ending position of this segment on each transcript.

TABLE 61
Segment location on transcripts

| | Segment | Segment |
|---|---|---|
| Transcript name | starting position | ending position |
| M78035_T0 (SEQ ID NO: 871) | 1593 | 1662 |
| M78035_T3 (SEQ ID NO: 872) | 1678 | 1747 |
| M78035_T4 (SEQ ID NO: 873) | 2274 | 2343 |
| M78035_T7 (SEQ ID NO: 874) | 1639 | 1708 |
| M78035_T9 (SEQ ID NO: 875) | 1851 | 1920 |
| M78035_T11 (SEQ ID NO: 876) | 1326 | 1395 |

Segment cluster M78035_node_{—}59 (SEQ ID NO:921) according to the present invention is supported by 251 libraries. The number of libraries was determined as previously described. This segment can be found in the following transcript(s): M78035_T0 (SEQ ID NO:871), M78035_T3 (SEQ ID NO:872), M78035_T4 (SEQ ID NO:873), M78035_T7 (SEQ ID NO:874), M78035_T9 (SEQ ID NO:875) and M78035_T11 (SEQ ID NO:876). Table 62 below describes the starting and ending position of this segment on each transcript.

TABLE 62
Segment location on transcripts

| | Segment | Segment |
|---|---|---|
| Transcript name | starting position | ending position |
| M78035_T0 (SEQ ID NO: 871) | 1814 | 1898 |
| M78035_T3 (SEQ ID NO: 872) | 1899 | 1983 |
| M78035_T4 (SEQ ID NO: 873) | 2495 | 2579 |
| M78035_T7 (SEQ ID NO: 874) | 1860 | 1944 |
| M78035_T9 (SEQ ID NO: 875) | 2072 | 2156 |
| M78035_T11 (SEQ ID NO: 876) | 1547 | 1631 |

### Variant Protein Alignment to the Previously Known Protein:

Sequence name: SAHH_HUMAN (SEQ ID NO:922)
Sequence documentation:
Alignment of: M78035_P4 (SEQ ID NO:924) × SAHH_HUMAN (SEQ ID NO:922) ..
Alignment segment 1/1:

| | | | |
|---|---|---|---|
| Quality: | 3949.00 | Escore | 0 |
| Matching length: | 404 | Total length: | 404 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

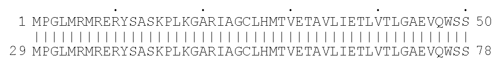

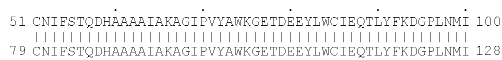

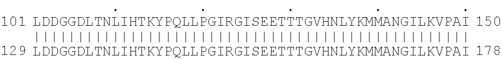

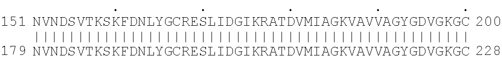

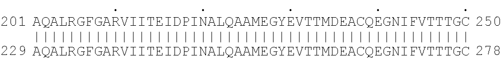

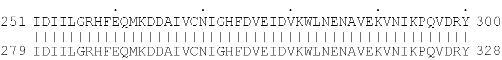

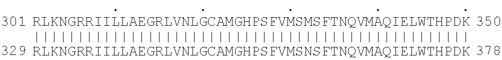

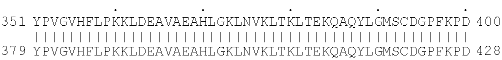

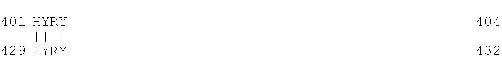

Sequence name: SAHH_HUMAN (SEQ ID NO:922)
Sequence documentation:
Alignment of: M78035_P6 (SEQ ID NO:925) × SAHH_HUMAN (SEQ ID NO:922) ..
Alignment segment 1/1:

| | | | |
|---|---|---|---|
| Quality: | 2982.00 | Escore | 0 |
| Matching length: | 306 | Total length: | 306 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

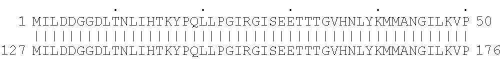

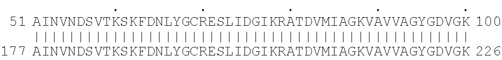

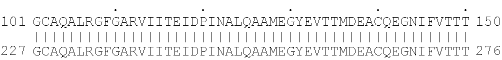

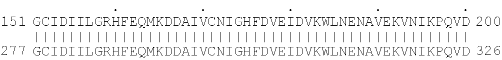

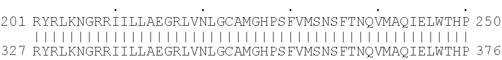

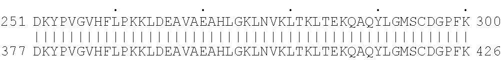

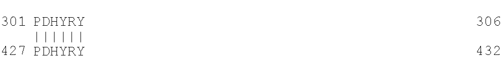

Sequence name: SAHH_HUMAN (SEQ ID NO:922)
Sequence documentation:
Alignment of: M78035_P8 (SEQ ID NO:926) × SAHH_HUMAN (SEQ ID NO:922) ..
Alignment segment 1/1:

| | | | |
|---|---|---|---|
| Quality: | 3275.00 | Escore | 0 |
| Matching length: | 334 | Total length: | 334 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

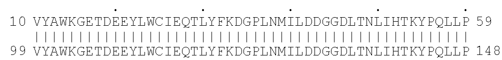

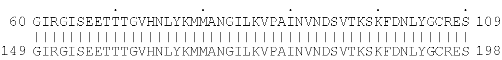

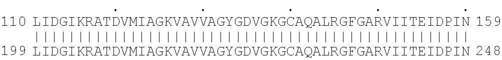

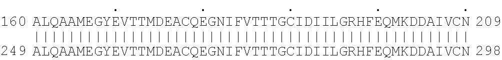

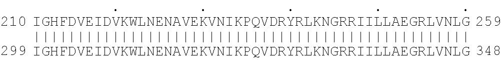

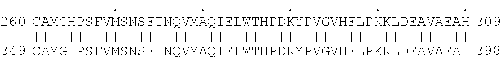

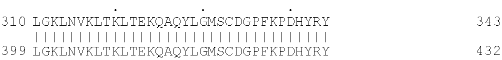

### Expression of S-Adenosylhomocysteine Hydrolase (AHCY) M78035 Transcripts which are Detectable by Amplicon as Depicted in Sequence Name M78035Seg42 (SEQ ID NO: 1351) in Normal and Cancerous Colon Tissues

Expression of S-adenosylhomocysteine hydrolase (AHCY) transcripts detectable by or according to seg42, M78035seg42 amplicon (SEQ ID NO: 1351) and M78035seg42F (SEQ ID NO: 1349) and M78035seg42R (SEQ ID NO: 1350) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

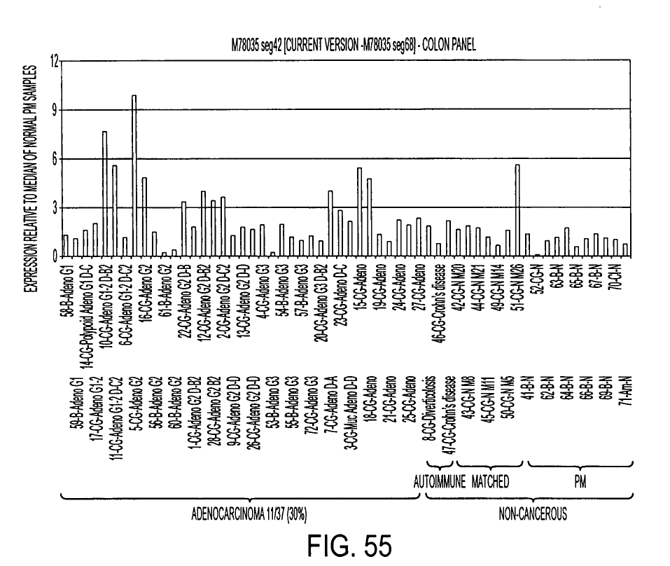

FIG. 55 is a histogram showing over expression of the above-indicated S-adenosylhomocysteine hydrolase (AHCY) transcripts in cancerous colon samples relative to the normal samples. The number and percentage of samples that exhibit at least 3 fold over-expression, out of the total number of samples tested is indicated in the bottom.

As is evident from FIG. 55, the expression of S-adenosylhomocysteine hydrolase (AHCY) transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 3 fold was found in 11 out of 37 adenocarcinoma samples, Statistical analysis was applied to verify the significance of these results, as described below.

The P value for the difference in the expression levels of S-adenosylhomocysteine hydrolase (AHCY) transcripts detectable by the above amplicon in colon cancer samples versus the normal tissue samples was determined by T test as 1.03E-04. Threshold of 3 fold overexpression was found to differentiate between cancer and normal samples with P value of 3.76E-02 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: M78035seg42F forward primer (SEQ ID NO: 1349); and M78035seg42R reverse primer (SEQ ID NO: 1350).

The present invention also preferably encompasses any amplicon obtained through the use of any suitable primer pair; for example, for the above experiment, the following amplicon was obtained as a non-limiting illustrative example only of a suitable amplicon: M78035seg42 (SEQ ID NO: 1351).

Forward primer (SEQ ID NO: 1349):
TGGTCTGGACTCAATCCCG
Reverse primer (SEQ ID NO: 1350):
GGAGTCTGAGTCCAAGCAGCC
Amplicon (SEQ ID NO: 1351):
TGGTCTGGACTCAATCCCGGGACTTTAGGACTTTTGCTAGAAATCTGGTG
TGGTGCAGGAGCGACTCCAGGATTCACTCTGTGGGCTGCTTGGACTCAGA
CTCC

### Description for Cluster R30650

Cluster R30650 features 8 transcript(s) and 49 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| R30650_PEA_2_T2 | 929 |
| R30650_PEA_2_T3 | 930 |
| R30650_PEA_2_T6 | 931 |
| R30650_PEA_2_T14 | 932 |
| R30650_PEA_2_T15 | 933 |
| R30650_PEA_2_T18 | 934 |
| R30650_PEA_2_T21 | 935 |
| R30650_PEA_2_T23 | 936 |

TABLE 2
Segments of interest

| Segment name | SEQ ID NO: |
|---|---|
| R30650_PEA_2_node_0 | 937 |
| R30650_PEA_2_node_1 | 938 |
| R30650_PEA_2_node_3 | 939 |
| R30650_PEA_2_node_5 | 940 |
| R30650_PEA_2_node_9 | 941 |
| R30650_PEA_2_node_11 | 942 |
| R30650_PEA_2_node_14 | 943 |
| R30650_PEA_2_node_20 | 944 |
| R30650_PEA_2_node_22 | 945 |
| R30650_PEA_2_node_24 | 946 |
| R30650_PEA_2_node_26 | 947 |
| R30650_PEA_2_node_32 | 948 |
| R30650_PEA_2_node_34 | 949 |
| R30650_PEA_2_node_36 | 950 |
| R30650_PEA_2_node_37 | 951 |
| R30650_PEA_2_node_39 | 952 |
| R30650_PEA_2_node_41 | 953 |
| R30650_PEA_2_node_42 | 954 |
| R30650_PEA_2_node_44 | 955 |
| R30650_PEA_2_node_46 | 956 |
| R30650_PEA_2_node_50 | 957 |
| R30650_PEA_2_node_56 | 958 |
| R30650_PEA_2_node_60 | 959 |
| R30650_PEA_2_node_63 | 960 |
| R30650_PEA_2_node_67 | 961 |
| R30650_PEA_2_node_70 | 962 |
| R30650_PEA_2_node_72 | 963 |
| R30650_PEA_2_node_73 | 964 |
| R30650_PEA_2_node_75 | 965 |
| R30650_PEA_2_node_79 | 966 |
| R30650_PEA_2_node_86 | 967 |
| R30650_PEA_2_node_87 | 968 |
| R30650_PEA_2_node_89 | 969 |
| R30650_PEA_2_node_93 | 970 |
| R30650_PEA_2_node_8 | 971 |
| R30650_PEA_2_node_17 | 972 |
| R30650_PEA_2_node_28 | 973 |
| R30650_PEA_2_node_31 | 974 |
| R30650_PEA_2_node_48 | 975 |
| R30650_PEA_2_node_53 | 976 |
| R30650_PEA_2_node_58 | 977 |
| R30650_PEA_2_node_68 | 978 |
| R30650_PEA_2_node_77 | 979 |
| R30650_PEA_2_node_82 | 980 |
| R30650_PEA_2_node_85 | 981 |
| R30650_PEA_2_node_88 | 982 |
| R30650_PEA_2_node_90 | 983 |
| R30650_PEA_2_node_91 | 984 |
| R30650_PEA_2_node_92 | 985 |

TABLE 3
Proteins of interest

| | SEQ | |
|---|---|---|
| | ID | |
| Protein Name | NO: | Corresponding Transcript(s) |
| R30650_PEA_2_P4 | 991 | R30650_PEA_2_T2 |
| | | (SEQ ID NO: 929) |
| R30650_PEA_2_P5 | 992 | R30650_PEA_2_T3 |
| | | (SEQ ID NO: 930) |
| R30650_PEA_2_P8 | 993 | R30650_PEA_2_T6 |
| | | (SEQ ID NO: 931) |
| R30650_PEA_2_P12 | 994 | R30650_PEA_2_T14 |
| | | (SEQ ID NO: 932) |
| R30650_PEA_2_P13 | 995 | R30650_PEA_2_T15 |
| | | (SEQ ID NO: 933); |
| | | R30650_PEA_2_T21 |
| | | (SEQ ID NO: 935) |
| R30650_PEA_2_P15 | 996 | R30650_PEA_2_T18 |
| | | (SEQ ID NO: 934) |
| R30650_PEA_2_P17 | 997 | R30650_PEA_2_T23 |
| | | (SEQ ID NO: 936) |

These sequences are variants of the known protein Protein KIAA1199 precursor (SwissProt accession identifier K199_HUMAN), SEQ ID NO: 986, referred to herein as the previously known protein.

Protein Protein KIAA1199 precursor (SEQ ID NO:986) is known or believed to have the following function(s): May be involved in hearing. The sequence for protein Protein KIAA1199 precursor is given at the end of the application, as “Protein KIAA1199 precursor amino acid sequence”. Known polymorphisms for this sequence are as shown in Table 4.

TABLE 4
Amino acid mutations for Known Protein

| SNP position(s) | |
|---|---|
| on amino acid | |
| sequence | Comment |
| 187 | R -> C (in nonsyndromic hearing loss; in one |
| | family). /FTId = VAR_018165. |
| 187 | R -> H (in nonsyndromic hearing loss; |
| | in two unrelated families). /FTId = VAR_018166. |
| 783 | H -> R. /FTId = VAR_018167. |
| 783 | H -> Y (in nonsyndromic hearing loss; in one |
| | sporadic case). /FTId = VAR_018168. |
| 1109 | V -> I. /FTId = VAR_018169. |
| 1169 | P -> A (common polymorphism). /FTId = |
| | VAR_018170. |
| 558-564 | HFHLAGD -> TRPPTRP |
| 862 | H -> T |

Cluster R30650 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

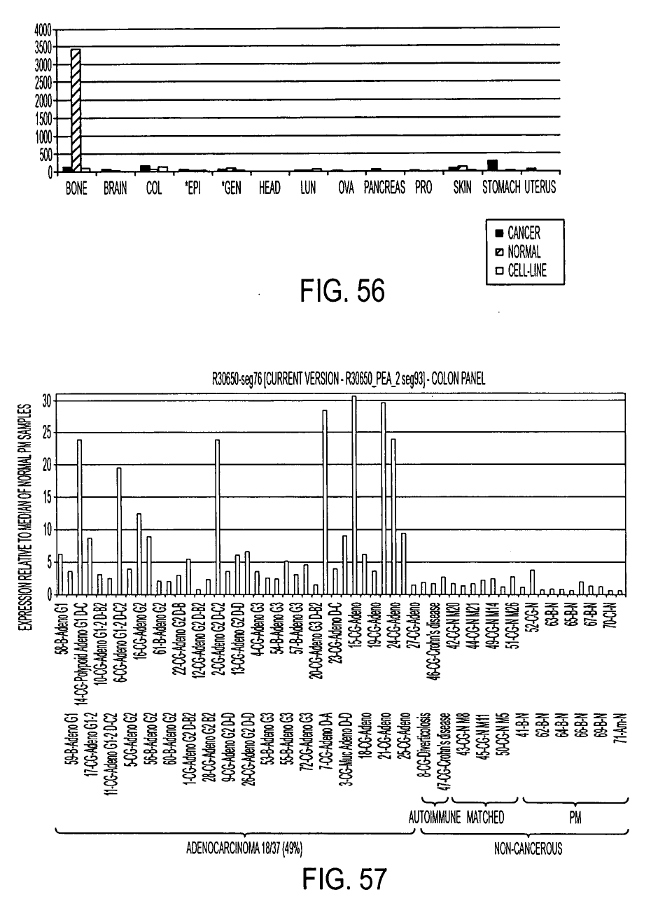

Overall, the following results were obtained as shown with regard to the histograms in FIG. 56 and Table 5. This cluster is overexpressed (at least at a minimum level) in the following pathological conditions: epithelial malignant tumors and a mixture of malignant tumors from different tissues.

TABLE 5
Normal tissue distribution

| Name of Tissue | Number |
|---|---|
| bone | 3420 |
| brain | 29 |
| colon | 63 |
| epithelial | 28 |
| general | 88 |
| head and neck | 0 |
| lung | 33 |
| ovary | 0 |
| pancreas | 0 |
| prostate | 2 |
| skin | 137 |
| stomach | 0 |
| uterus | 0 |

TABLE 6
P values and ratios for expression in cancerous tissue

| Name of Tissue | P1 | P2 | SP1 | R3 | SP2 | R4 |
|---|---|---|---|---|---|---|
| bone | 6.9e−01 | 7.8e−01 | 1 | 0.0 | 1 | 0.0 |
| brain | 2.7e−01 | 5.0e−01 | 8.8e−02 | 2.3 | 3.7e−01 | 1.3 |
| colon | 6.7e−02 | 4.8e−02 | 1.6e−01 | 1.8 | 1.8e−01 | 1.8 |
| epithelial | 2.7e−03 | 7.1e−03 | 8.0e−03 | 1.6 | 1.0e−01 | 1.3 |
| general | 7.9e−03 | 5.0e−02 | 1 | 0.6 | 1 | 0.4 |
| head and neck | 2.1e−01 | 3.3e−01 | 0.0e+00 | 0.0 | 0.0e+00 | 0.0 |
| lung | 5.7e−01 | 3.5e−01 | 8.8e−01 | 0.8 | 3.4e−01 | 1.3 |
| ovary | 2.2e−01 | 2.6e−01 | 4.7e−01 | 2.0 | 5.9e−01 | 1.7 |
| pancreas | 2.2e−02 | 6.9e−02 | 3.2e−02 | 6.5 | 7.7e−02 | 4.6 |
| prostate | 9.0e−01 | 9.2e−01 | 4.5e−01 | 1.8 | 5.6e−01 | 1.5 |
| skin | 6.0e−01 | 5.8e−01 | 8.1e−01 | 0.6 | 1 | 0.3 |
| stomach | 3.0e−01 | 4.3e−01 | 4.0e−03 | 3.0 | 8.4e−02 | 2.3 |
| uterus | 4.1e−02 | 1.6e−01 | 8.5e−02 | 3.6 | 2.6e−01 | 2.3 |

For this cluster, at least one oligonucleotide was found to demonstrate overexpression of the cluster, although not of at least one transcript/segment as listed below. Microarray (chip) data is also available for this cluster as follows. Various oligonucleotides were tested for being differentially expressed in various disease conditions, particularly cancer, as previously described. The following oligonucleotides were found to hit this cluster but not other segments/transcripts below, shown in Table 7.

TABLE 7
Oligonucleotides related to this cluster

| Oligonucleotide name | Overexpressed in cancers | Chip reference |
|---|---|---|
| H85953_0_18_0 | colorectal cancer | Colon |

As noted above, cluster R30650 features 8 transcript(s), which were listed in Table 1 above. These transcript(s) encode for protein(s) which are variant(s) of protein Protein KIAA1199 precursor (SEQ ID NO:986). A description of each variant protein according to the present invention is now provided.

Variant protein R30650_PEA_{—}2_P4 (SEQ ID NO:991) according to the present invention has an amino acid sequence as given at the end of the application; it is encoded by transcript(s) R30650_PEA_{—}2_T2 (SEQ ID NO:929). An alignment is given to the known protein (Protein KIAA1199 precursor (SEQ ID NO:986)) at the end of the application. One or more alignments to one or more previously published protein sequences are given at the end of the application. A brief description of the relationship of the variant protein according to the present invention to each such aligned protein is as follows:

### Comparison Report Between R30650_PEA_{—}2_P4 (SEQ ID NO:991) and Q9ULM1 (SEQ ID NO:989):

1. An isolated chimeric polypeptide encoding for R30650_PEA_{—}2_P4 (SEQ ID NO:991), comprising a first amino acid sequence being at least 90% homologous to MYLHIGEEIDGVDMRAEVGLLSRNIIVMGEMEDKCYPYRNHICNFFDFDTFGGHIKFALGFKAAHLEGTEL KHMGQQLVGQYPIHFHLAGDVDERGGYDPPTYIRDLSIHHTFSRCVTVHGSNGLLIKDVVGYNSLGHCFF TEDGPEERNTFDHCLGLLVKSGTLLPSDRDSKMCKMITEDSYPGYIPKPRQDCNAVSTFWMANPNNNLIN CAAAGSEETGFWFIFHHVPTGPSVGMYSPGYSEHIPLGKFYNNRAHSNYRAGMIIDNGVKTTEASAKDKR PFLSIISARYSPHQDADPLKPREPAIIRHFIAYKNQDHGAWLRGGDVWLDSCRFADNGIGLTLASGGTFPYD DGSKQEIKNSLFVGESGNVGTEMMDNRIWGPGGLDHSGRTLPIGQNFPIRGIQLYDGPINIQNCTFRKFVAL EGRHTSALAFRLNNAWQSCPHNNVTGIAFEDVPITSRVFFGEPGPWFNQLDMDGDKTSVFHDVDGSVSEY PGSYLTKNDNWLVRHPDCINVPDWRGAICSGCYAQMYIQAYKTSNLRMKIIKNDFPSHPLYLEGALTRST HYQQYQPVVTLQKGYTIHWDQTAPAELAIWLINFNKGDWIRVGLCYPRGTTFSILSDVHNRLLKQTSKTG VFVRTLQMDKVEQSYPGRSHYYWDEDSGLLFLKLKAQNEREKFAFCSMKGCERIKIKALIPKNAGVSDCT ATAYPKFTERAVVDVPMPKKLFGSQLKTKDHFLEVKMESSKQHFFHLWNDFAYIEVDGKKYPSSEDGIQV VVIDGNQGRVVSHTSFRNSILQGIPWQLFNYVATIPDNSIVLMASKGRYVSRGPWTRVLEKLGADRGLKL KEQMAFVGFKGSFRPIWVTLDTEDHKAKIFQVVPIPVVKKKKL corresponding to amino acids 126-1013 of Q9ULM1 (SEQ ID NO:989), which also corresponds to amino acids 1-888 of R30650_PEA_{—}2_P4 (SEQ ID NO:991).

Comparison Report Between R30650_PEA_{—}2_P4 (SEQ ID NO:991) and Q8WUJ3 (SEQ ID NO: 987):

1. An isolated chimeric polypeptide encoding for R30650_PEA_{—}2_P4 (SEQ ID NO:991), comprising a first amino acid sequence being at least 90% homologous to MYLHIGEEIDGVDMRAEVGLLSRNIIVMGEMEDKCYPYRNHICNFFDFDTFGGHIKFALGFKAAHLEGTEL KHMGQQLVGQYPIHFHLAGDVDERGGYDPPTYIRDLSIHHTFSRCVTVHGSNGLLIKDVVGYNSLGHCFF TEDGPEERNTFDHCLGLLVKSGTLLPSDRDSKMCKMITEDSYPGYIPKPRQDCNAVSTFWMANPNNNLIN CAAAGSEETGFWFIFHHVPTGPSVGMYSPGYSEHIPLGKFYNNRAHSNYRAGMIIDNGVKTTEASAKDKR PFLSIISARYSPHQDADPLKPREPAIIRHFIAYKNQDHGAWLRGGDVWLDSCRFADNGIGLTLASGGTFPYD DGSKQEIKNSLFVGESGNVGTEMMDNRIWGPGGLDHSGRTLPIGQNFPIRGIQLYDGPINIQNCTFRKFVAL EGRHTSALAFRLNNAWQSCPHNNVTGIAFEDVPITSRVFFGEPGPWFNQLDMDGDKTSVFHDVDGSVSEY PGSYLTKND corresponding to amino acids 474-977 of Q8WUJ3 (SEQ ID NO:987), which also corresponds to amino acids 1-504 of R30650_PEA_{—}2_P4 (SEQ ID NO:991), and a second amino acid sequence being at least 70%, optionally at least 80%, preferably at least 85%, more preferably at least 90% and most preferably at least 95% homologous to a polypeptide having the sequence NWLVRHPDCINVPDWRGAICSGCYAQMYIQAYKTSNLRMKIIKNDFPSHPLYLEGALTRSTHYQQYQPVV TLQKGYTIHWDQTAPAELAIWLINFNKGDWIRVGLCYPRGTTFSILSDVHNRLLKQTSKTGVFVRTLQMD KVEQSYPGRSHYYWDEDSGLLFLKLKAQNEREKFAFCSMKGCERIKIKALIPKNAGVSDCTATAYPKFTE RAVVDVPMPKKLFGSQLKTKDHFLEVKMESSKQHFFHLWNDFAYIEVDGKKYPSSEDGIQVVVIDGNQG RVVSHTSFRNSILQGIPWQLFNYVATIPDNSIVLMASKGRYVSRGPWTRVLEKLGADRGLKLKEQMAFVG FKGSFRPIWVTLDTEDHKAKIFQVVPIPVVKKKKL corresponding to amino acids 505-888 of R30650_PEA_{—}2_P4 (SEQ ID NO:991), wherein said first amino acid sequence and second amino acid sequence are contiguous and in a sequential order.

2. An isolated polypeptide encoding for a tail of R30650_PEA_{—}2_P4 (SEQ ID NO:991), comprising a polypeptide being at least 70%, optionally at least about 80%, preferably at least about 85%, more preferably at least about 90% and most preferably at least about 95% homologous to the sequence NWLVRHPDCINVPDWRGAICSGCYAQMYIQAYKTSNLRMKIIKNDFPSHPLYLEGALTRSTHYQQYQPVV TLQKGYTIHWDQTAPAELAIWLINFNKGDWIRVGLCYPRGTTFSILSDVHNRLLKQTSKTGVFVRTLQMD KVEQSYPGRSHYYWDEDSGLLFLKLKAQNEREKFAFCSMKGCERIKIKALIPKNAGVSDCTATAYPKFTE RAYVDVPMPKKLFGSQLKTKDHFLEVKMESSKQHFFHLWNDFAYIEVDGKKYPSSEDGIQVVVIDGNQG RVVSHTSFRNSILQGIPWQLFNYVATIPDNSIVLMASKGRYVSRGPWTRVLEKLGADRGLKLKEQMAFVG FKGSFRPIWVTLDTEDHKAKIFQVVPIPVVKKKKL in R30650_PEA_{—}2_P4 (SEQ ID NO:991).

Comparison Report Between R30650_PEA_{—}2_P4 (SEQ ID NO:991) and Q9NPN9 (SEQ ID NO: 988):

1. An isolated chimeric polypeptide encoding for R30650_PEA_{—}2_P4 (SEQ ID NO:991), comprising a first amino acid sequence being at least 70%, optionally at least 80%, preferably at least 85%, more preferably at least 90% and most preferably at least 95% homologous to a polypeptide having the sequence MYLHIGEEIDGVDMRAEVGLLSRNIIVMGEMEDKCYPYRNHICNFFDFDTFGGHIKFALGFKAAHLEGTEL KHMGQQLVGQYPIHFHLAGD corresponding to amino acids 1-91 of R30650_PEA_{—}2_P4 (SEQ ID NO:991), and a second amino acid sequence being at least 90% homologous to VDERGGYDPPTYIRDLSIHHTFSRCVTVHGSNGLLIKDVVGYNSLGHCFFTEDGPEERNTFDHCLGLLVKS GTLLPSDRDSKMCKMITEDSYPGYIPKPRQDCNAVSTFWMANPNNNLINCAAAGSEETGFWFIFHHVPTG PSVGMYSPGYSEHIPLGKFYNNRAHSNYRAGMIIDNGVKTTEASAKDKRPFLSIISARYSPHQDADPLKPRE PAIIRHFIAYKNQDHGAWLRGGDVWLDSCRFADNGIGLTLASGGTFPYDDGSKQEIKNSLFVGESGNVGT EMMDNRIWGPGGLDHSGRTLPIGQNFPIRGIQLYDGPINIQNCTFRKFVALEGRHTSALAFRLNNAWQSCP HNNVTGIAFEDVPITSRVFFGEPGPWFNQLDMDGDKTSVFHDVDGSVSEYPGSYLTKNDNWLVRHPDCIN VPDWRGAICSGCYAQMYIQAYKTSNLRMKIIKNDFPSHPLYLEGALTRSTHYQQYQPVVTLQKGYTIHWD QTAPAELAIWLINFNKGDWIRVGLCYPRGTTFSILSDVHNRLLKQTSKTGVFVRTLQMDKVEQSYPGRSH YYWDEDSGLLFLKLKAQNEREKFAFCSMKGCERIKIKALIPKNAGVSDCTATAYPKFTERAVVDVPMPKK LFGSQLKTKDHFLEVKMESSKQHFFHLWNDFAYIEVDGKKYPSSEDGIQVVVIDGNQGRVVSHTSFRNSIL QGIPWQLFNYVATIPDNSIVLMASKGRYVSRGPWTRVLEKLGADRGLKLKEQMAFVGFKGSFRPIWVTLD TEDHKAKIFQVVPIPVVKKKKL corresponding to amino acids 8-804 of Q9NPN9 (SEQ ID NO:988), which also corresponds to amino acids 92-888 of R30650_PEA_{—}2_P4 (SEQ ID NO:991), wherein said first amino acid sequence and second amino acid sequence are contiguous and in a sequential order.

2. An isolated polypeptide encoding for a head of R30650_PEA_{—}2_P4 (SEQ ID NO:991), comprising a polypeptide being at least 70%, optionally at least about 80%, preferably at least about 85%, more preferably at least about 90% and most preferably at least about 95% homologous to the sequence MYLHIGEEIDGVDMRAEVGLLSRNIIVMGEMEDKCYPYRNHICNFFDFDTFGGHIKFALGFKAAHLEGTEL KHMGQQLVGQYPIHFHLAGD of R30650_PEA_{—}2_P4 (SEQ ID NO:991).

Comparison Report Between R30650_PEA_{—}2_P4 (SEQ ID NO:991) and Q9H1K5 (SEQ ID NO:990):

1. An isolated chimeric polypeptide encoding for R30650_PEA_{—}2_P4 (SEQ ID NO:991), comprising a first amino acid sequence being at least 70%, optionally at least 80%, preferably at least 85%, more preferably at least 90% and most preferably at least 95% homologous to a polypeptide having the sequence MYLHIGEEIDGVDMRAEVGLLSRNIIVMGEMEDKCYPYRNHICNFFDFDTFGGHIKFALGFKAAHLEGTEL KHMGQQLVGQYPIHFHLAGDVDERGGYDPPTYIRDLSIHHTFSRCVTVHGSNGLLIKDVVGYNSLGHCFF TEDGPEERNTFDHCLGLLVKSGTLLPSDRDSKMCKMITEDSYPGYIPKPRQDCNAVSTFWMANPNNNLIN CAAAGSEETGFWFIFHHVPTGPSVGMYSPGYSEHIPLGKFYNNRAHSNYRAGMIIDNGVKTTEASAKDKR PFLSIISARYSPHQDADPLKPREPAIIRHFIAYKNQDHGAWLRGGDVWLDSCRFADNGIGLTLASGGTFPYD DGSKQEIKNSLFVGESGNVGTEMMDNRIWGPGGLDH corresponding to amino acids 1-389 of R30650_PEA_{—}2_P4 (SEQ ID NO:991), and a second amino acid sequence being at least 90% homologous to SGRTLPIGQNFPIRGIQLYDGPINIQNCTFRKFVALEGRHTSALAFRLNNAWQSCPHNNVTGIAFEDVPITSR VFFGEPGPWFNQLDMDGDKTSVFHDVDGSVSEYPGSYLTKNDNWLVRHPDCINVPDWRGAICSGCYAQ MYIQAYKTSNLRMKIIKNDFPSHPLYLEGALTRSTHYQQYQPVVTLQKGYTIHWDQTAPAELAIWLINFNK GDWIRVGLCYPRGTTFSILSDVHNRLLKQTSKTGVFVRTLQMDKVEQSYPGRSHYYWDEDSGLLFLKLKA QNEREKFAFCSMKGCERIKIKALIPKNAGVSDCTATAYPKFTERAVVDVPMPKKLFGSQLKTKDHFLEVK MESSKQHFFHLWNDFAYIEVDGKKYPSSEDGIQVVVIDGNQGRVVSHTSFRNSILQGIPWQLFNYVATIPD NSIVLMASKGRYVSRGPWTRVLEKLGADRGLKLKEQMAFVGFKGSFRPIWVTLDTEDHKAKIFQVVPIPV VKKKKL corresponding to amino acids 2-500 of Q9H1K5 (SEQ ID NO:990), which also corresponds to amino acids 390-888 of R30650_PEA_{—}2_P4 (SEQ ID NO:991), wherein said first amino acid sequence and second amino acid sequence are contiguous and in a sequential order.

2. An isolated polypeptide encoding for a head of R30650_PEA_{—}2_P4 (SEQ ID NO:991), comprising a polypeptide being at least 70%, optionally at least about 80%, preferably at least about 85%, more preferably at least about 90% and most preferably at least about 95% homologous to the sequence MYLHIGEEIDGVDMRAEVGLLSRNIIVMGEMEDKCYPYRNHICNFFDFDTFGGHIKFALGFKAAHLEGTEL KHMGQQLVGQYPIHFHLAGDVDERGGYDPPTYIRDLSIHHTFSRCVTVHGSNGLLIKDVVGYNSLGHCFF TEDGPEERNTFDHCLGLLVKSGTLLPSDRDSKMCKMITEDSYPGYIPKPRQDCNAVSTFWMANPNNNLIN CAAAGSEETGFWFIFHHVPTGPSVGMYSPGYSEHIPLGKFYNNRAHSNYRAGMIIDNGVKTTEASAKDKR PFLSIISARYSPHQDADPLKPREPAIIRHFIAYKNQDHGAWLRGGDVWLDSCRFADNGIGLTLASGGTFPYD DGSKQEIKNSLFVGESGNVGTEMMDNRIWGPGGLDH of R30650_PEA_{—}2_P4 (SEQ ID NO:991).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein R30650_PEA_{—}2_P4 (SEQ ID NO:991) also has the following non-silent SNPs (Single Nucleotide Polymorphisms) as listed in Table 8, (given according to their position(s) on the amino acid sequence, with the alternative amino acid(s) listed; the last column indicates whether the SNP is known or not; the presence of known SNPs in variant protein R30650_PEA_{—}2_P4 (SEQ ID NO:991) sequence provides support for the deduced sequence of this variant protein according to the present invention).

TABLE 8
Amino acid mutations

| SNP position(s) on amino acid | | Previously |
|---|---|---|
| sequence | Alternative amino acid(s) | known SNP? |
| 264 | M -> V | Yes |
| 310 | H -> R | Yes |

Variant protein R30650_PEA_{—}2_P4 (SEQ ID NO:991) is encoded by the following transcript(s): R30650_PEA_{—}2_T2 (SEQ ID NO:929), for which the sequence(s) is/are given at the end of the application. The coding portion of transcript R30650_PEA_{—}2_T2 (SEQ ID NO:929) is shown in bold; this coding portion starts at position 1369 and ends at position 4032. The transcript also has the following SNPs as listed in Table 9 (given according to their position on the nucleotide sequence, with the alternative nucleic acid listed; the last column indicates whether the SNP is known or not; the presence of known SNPs in variant protein R30650_PEA_{—}2_P4 (SEQ ID NO:991) sequence provides support for the deduced sequence of this variant protein according to the present invention).

TABLE 9
Nucleic acid SNPs

| SNP position on nucleotide | | Previously |
|---|---|---|
| sequence | Alternative nucleic acid | known SNP? |
| 464 | T -> C | Yes |
| 1833 | T -> C | Yes |
| 1920 | A -> G | Yes |
| 2158 | A -> G | Yes |
| 2297 | A -> G | Yes |
| 4180 | G -> C | Yes |
| 4396 | G -> A | Yes |
| 5457 | G -> A | Yes |
| 6505 | C -> T | Yes |
| 6644 | T -> C | Yes |
| 6736 | A -> C | Yes |

Variant protein R30650_PEA_{—}2_P5 (SEQ ID NO:992) according to the present invention has an amino acid sequence as given at the end of the application; it is encoded by transcript(s) R30650_PEA_{—}2_T3 (SEQ ID NO:930). An alignment is given to the known protein (Protein KIAA1199 precursor (SEQ ID NO:986)) at the end of the application. One or more alignments to one or more previously published protein sequences are given at the end of the application. A brief description of the relationship of the variant protein according to the present invention to each such aligned protein is as follows:

Comparison Report Between R30650_PEA_{—}2_P5 (SEQ ID NO:992) and Q9ULM1 (SEQ ID NO:989):

1. An isolated chimeric polypeptide encoding for R30650_PEA_{—}2_P5 (SEQ ID NO:992), comprising a first amino acid sequence being at least 90% homologous to MDGVNLSTEVVYKKGQDYRFACYDRGRACRSYRVRFLCGKPVRPKLTVTIDTNVNSTILNLEDNVQSWK PGDTLVIASTDYSMYQAEEFQVLPCRSCAPNQVKVAGKPMYLHIGEEIDGVDMRAEVGLLSRNIIVMGEM EDKCYPYRNHICNFFDFDTFGGHIKFALGFKAAHLEGTELKHMGQQLVGQYPIHFHLAGDVDERGGYDPP TYIRDLSIHHTFSRCVTVHGSNGLLIKDVVGYNSLGHCFFTEDGPEERNTFDHCLGLLVKSGTLLPSDRDSK MCKMITEDSYPGYIPKPRQDCNAVSTFWMANPNNNLINCAAAGSEETGFWFIFHHVPTGPSVGMYSPGYS EHIPLGKFYNNRAHSNYRAGMIIDNGVKTTEASAKDKRPFLSIISARYSPHQDADPLKPREPAIIRHFIAYKN QDHGAWLRGGDVWLDSCRFADNGIGLTLASGGTFPYDDGSKQEIKNSLFVGESGNVGTEMMDNRIWGP GGLDHSGRTLPIGQNFPIRGIQLYDGPINIQNCTFRKFVALEGRHTSALAFRLNNAWQSCPHNNVTGIAFED VPITSRVFFGEPGPWFNQLDMDGDKTSVFHDVDGSVSEYPGSYLTKNDNWLVRHPDCINVPDWRGAICSG CYAQMYIQAYKTSNLRMKIIKNDFPSHPLYLEGALTRSTHYQQYQPVVTLQKGYTIHWDQTAPAELAIWL INFNKGDWIRVGLCYPRGTTFSILSDVHNRLLKQTSKTGVFVRTLQMDKVEQSYPGRSHYYWDEDSGLLF LKLKAQNEREKFAFCSMKGCERIKIKALIPKNAGVSDCTATAYPKFTERAVVDVPMPKKLFGSQLKTKDH FLEVKMESSKQHFFHLWNDFAYIEVDGKKYPSSEDGIQVVVIDGNQGRVVSHTSFRNSILQGIPWQLFNYV ATIPDNSIVLMASKGRYVSRGPWTRVLEKLGADRGLKLKEQMAFVGFKGSFRPIWVTLDTEDHKAKIFQV VPIPVVKKKKL corresponding to amino acids 18-1013 of Q9ULM1 (SEQ ID NO:989), which also corresponds to amino acids 1-996 of R30650_PEA_{—}2_P5 (SEQ ID NO:992).

Comparison Report Between R30650_PEA_{—}2_P5 (SEQ ID NO:992) and Q8WUJ3 (SEQ ID NO:987):

1. An isolated chimeric polypeptide encoding for R30650_PEA_{—}2_P5 (SEQ ID NO:992), comprising a first amino acid sequence being at least 90% homologous to MDGVNLSTEVVYKKGQDYRFACYDRGRACRSYRVRFLCGKPVRPKLTVTIDTNVNSTILNLEDNVQSWK PGDTLVIASTDYSMYQAEEFQVLPCRSCAPNQVKVAGKPMYLHIGEEIDGVDMRAEVGLLSRNIIVMGEM EDKCYPYRNHICNFFDFDTFGGHIKFALGFKAAHLEGTELKHMGQQLVGQYPIHFHLAGDVDERGGYDPP TYIRDLSIHHTFSRCVTVHGSNGLLIKDVVGYNSLGHCFFTEDGPEERNTFDHCLGLLVKSGTLLPSDRDSK MCKMITEDSYPGYIPKPRQDCNAVSTFWMANPNNNLINCAAAGSEETGFWFIFHHVPTGPSVGMYSPGYS EHIPLGKFYNNRAHSNYRAGMIIDNGVKTTEASAKDKRPFLSIISARYSPHQDADPLKPREPAIIRHFIAYKN QDHGAWLRGGDVWLDSCRFADNGIGLTLASGGTFPYDDGSKQEIKNSLFVGESGNVGTEMMDNRIWGP GGLDHSGRTLPIGQNFPIRGIQLYDGPINIQNCTFRKFVALEGRHTSALAFRLNNAWQSCPHNNVTGIAFED VPITSRVFFGEPGPWFNQLDMDGDKTSVFHDVDGSVSEYPGSYLTKND corresponding to amino acids 366-977 of Q8WUJ3 (SEQ ID NO:987), which also corresponds to amino acids 1-612 of R30650_PEA_{—}2_P5 (SEQ ID NO:992), and a second amino acid sequence being at least 70%, optionally at least 80%, preferably at least 85%, more preferably at least 90% and most preferably at least 95% homologous to a polypeptide having the sequence NWLVRHPDCINVPDWRGAICSGCYAQMYIQAYKTSNLRMKIIKNDFPSHPLYLEGALTRSTHYQQYQPVV TLQKGYTIHWDQTAPAELAIWLINFNKGDWIRVGLCYPRGTTFSILSDVHNRLLKQTSKTGVFVRTLQMD KVEQSYPGRSHYYWDEDSGLLFLKLKAQNEREKFAFCSMKGCERIKIKALIPKNAGVSDCTATAYPKFTE RAVVDVPMPKKLFGSQLKTKDHFLEVKMESSKQHFFHLWNDFAYIEVDGKKYPSSEDGIQVVVIDGNQG RVVSHTSFRNSILQGIPWQLFNYVATIPDNSIVLMASKGRYVSRGPWTRVLEKLGADRGLKLKEQMAFVG FKGSFRPIWVTLDTEDHKAKIFQVVPIPVVKKKKL corresponding to amino acids 613-996 of R30650_PEA_{—}2_P5 (SEQ ID NO:992), wherein said first amino acid sequence and second amino acid sequence are contiguous and in a sequential order.

2. An isolated polypeptide encoding for a tail of R30650_PEA_{—}2_P5 (SEQ ID NO:992), comprising a polypeptide being at least 70%, optionally at least about 80%, preferably at least about 85%, more preferably at least about 90% and most preferably at least about 95% homologous to the sequence NWLVRHPDCINVPDWRGAICSGCYAQMYIQAYKTSNLRMKIIKNDFPSHPLYLEGALTRSTHYQQYQPVV TLQKGYTIHWDQTAPAELAIWLINFNKGDWIRVGLCYPRGTTFSILSDVHNRLLKQTSKTGVFVRTLQMD KVEQSYPGRSHYYWDEDSGLLFLKLKAQNEREKFAFCSMKGCERIKIKALIPKNAGVSDCTATAYPKFTE RAVVDVPMPKKLFGSQLKTKDHFLEVKMESSKQHFFHLWNDFAYIEVDGKKYPSSEDGIQVVVIDGNQG RVVSHTSFRNSILQGIPWQLFNYVATIPDNSIVLMASKGRYVSRGPWTRVLEKLGADRGLKLKEQMAFVG FKGSFRPIWVTLDTEDHKAKIFQVVPIPVVKKKKL in R30650_PEA_{—}2_P5 (SEQ ID NO:992).

Comparison Report Between R30650_PEA_{—}2_P5 (SEQ ID NO:992) and Q9NPN9 (SEQ ID NO:988):

1. An isolated chimeric polypeptide encoding for R30650_PEA_{—}2_P5 (SEQ ID NO:992), comprising a first amino acid sequence being at least 70%, optionally at least 80%, preferably at least 85%, more preferably at least 90% and most preferably at least 95% homologous to a polypeptide having the sequence MDGVNLSTEVVYKKGQDYRFACYDRGRACRSYRVRFLCGKPVRPKLTVTIDTNVNSTILNLEDNVQSWK PGDTLVIASTDYSMYQAEEFQVLPCRSCAPNQVKVAGKPMYLHIGEEIDGVDMRAEVGLLSRNIIVMGEM EDKCYPYRNHICNFFDFDTFGGHIKFALGFKAAHLEGTELKHMGQQLVGQYPIHFHLAGD (SEQ ID NO:1468) corresponding to amino acids 1-199 of R30650_PEA_{—}2_P5 (SEQ ID NO:992), and a second amino acid sequence being at least 90% homologous to VDERGGYDPPTYIRDLSIHHTFSRCVTVHGSNGLLIKDVVGYNSLGHCFFTEDGPEERNTFDHCLGLLVKS GTLLPSDRDSKMCKMITEDSYPGYIPKPRQDCNAVSTFWMANPNNNLINCAAAGSEETGFWFIFHHVPTG PSVGMYSPGYSEHIPLGKFYNNRAHSNYRAGMIIDNGVKTTEASAKDKRPFLSIISARYSPHQDADPLKPRE PAIIRHFIAYKNQDHGAWLRGGDVWLDSCRFADNGIGLTLASGGTFPYDDGSKQEIKNSLFVGESGNVGT EMMDNRIWGPGGLDHSGRTLPIGQNFPIRGIQLYDGPINIQNCTFRKFVALEGRHTSALAFRLNNAWQSCP HNNVTGIAFEDVPITSRVFFGEPGPWFNQLDMDGDKTSVFHDVDGSVSEYPGSYLTKNDNWLVRHPDCIN VPDWRGAICSGCYAQMYIQAYKTSNLRMKIIKNDFPSHPLYLEGALTRSTHYQQYQPVVTLQKGYTIHWD QTAPAELAIWLINFNKGDWIRVGLCYPRGTTFSILSDVHNRLLKQTSKTGVFVRTLQMDKVEQSYPGRSH YYWDEDSGLLFLKLKAQNEREKFAFCSMKGCERIKIKALIPKNAGVSDCTATAYPKFTERAVVDVPMPKK LFGSQLKTKDHFLEVKMESSKQHFFHLWNDFAYIEVDGKKYPSSEDGIQVVVIDGNQGRVVSHTSFRNSIL QGIPWQLFNYVATIPDNSIVLMASKGRYVSRGPWTRVLEKLGADRGLKLKEQMAFVGFKGSFRPIWVTLD TEDHKAKIFQVVPIPVVKKKKL corresponding to amino acids 8-804 of Q9NPN9 (SEQ ID NO:988), which also corresponds to amino acids 200-996 of R30650_PEA_{—}2_P5 (SEQ ID NO:992), wherein said first amino acid sequence and second amino acid sequence are contiguous and in a sequential order.

2. An isolated polypeptide encoding for a head of R30650_PEA_{—}2_P5 (SEQ ID NO:992), comprising a polypeptide being at least 70%, optionally at least about 80%, preferably at least about 85%, more preferably at least about 90% and most preferably at least about 95% homologous to the sequence MDGVNLSTEVVYKKGQDYRFACYDRGRACRSYRVRFLCGKPVRPKLTVTIDTNVNSTILNLEDNVQSWK PGDTLVIASTDYSMYQAEEFQVLPCRSCAPNQVKVAGKPMYLHIGEEIDGVDMRAEVGLLSRNIIVMGEM EDKCYPYRNHICNFFDFDTFGGHIKFALGFKAAHLEGTELKHMGQQLVGQYPIHFHLAGD (SEQ ID NO:1468) of R30650_PEA_{—}2_P5 (SEQ ID NO:992).

Comparison Report Between R30650_PEA_{—}2_P5 (SEQ ID NO:992) and Q9H1K5 (SEQ ID NO:990):

1. An isolated chimeric polypeptide encoding for R30650_PEA_{—}2_P5 (SEQ ID NO:992), comprising a first amino acid sequence being at least 70%, optionally at least 80%, preferably at least 85%, more preferably at least 90% and most preferably at least 95% homologous to a polypeptide having the sequence MDGVNLSTEVVYKKGQDYRFACYDRGRACRSYRVRFLCGKPVRPKLTVTIDTNVNSTILNLEDNVQSWK PGDTLVIASTDYSMYQAEEFQVLPCRSCAPNQVKVAGKPMYLHIGEEIDGVDMRAEVGLLSRNIIVMGEM EDKCYPYRNHICNFFDFDTFGGHIKFALGFKAAHLEGTELKHMGQQLVGQYPIHFHLAGDVDERGGYDPP TYIRDLSIHHTFSRCVTVHGSNGLLIKDVVGYNSLGHCFFTEDGPEERNTFDHCLGLLVKSGTLLPSDRDSK MCKMITEDSYPGYIPKPRQDCNAVSTFWMANPNNNLINCAAAGSEETGFWFIFHHVPTGPSVGMYSPGYS EHIPLGKFYNNRAHSNYRAGMIIDNGVKTTEASAKDKRPFLSIISARYSPHQDADPLKPREPAIIRHFIAYKN QDHGAWLRGGDVWLDSCRFADNGIGLTLASGGTFPYDDGSKQEIKNSLFVGESGNVGTEMMDNRIWGP GGLDH corresponding to amino acids 1-497 of R30650_PEA_{—}2_P5 (SEQ ID NO:992), and a second amino acid sequence being at least 90% homologous to SGRTLPIGQNFPIRGIQLYDGPINIQNCTFRKFVALEGRHTSALAFRLNNAWQSCPHNNVTGIAFEDVPITSR VFFGEPGPWFNQLDMDGDKTSVFHDVDGSVSEYPGSYLTKNDNWLVRHPDCINVPDWRGAICSGCYAQ MYIQAYKTSNLRMKIIKNDFPSHPLYLEGALTRSTHYQQYQPVVTLQKGYTIHWDQTAPAELAIWLINFNK GDWIRVGLCYPRGTTFSILSDVHNRLLKQTSKTGVFVRTLQMDKVEQSYPGRSHYYWDEDSGLLFLKLKA QNEREKFAFCSMKGCERIKIKALIPKNAGVSDCTATAYPKFTERAVVDVPMPKKLFGSQLKTKDHFLEVK MESSKQHFFHLWNDFAYIEVDGKKYPSSEDGIQVVVIDGNQGRVVSHTSFRNSILQGIPWQLFNYVATIPD NSIVLMASKGRYVSRGPWTRVLEKLGADRGLKLKEQMAFVGFKGSFRPIWVTLDTEDHKAKIFQVVPIPV VKKKKL corresponding to amino acids 2-500 of Q9H1K5 (SEQ ID NO:990), which also corresponds to amino acids 498-996 of R30650_PEA_{—}2_P5 (SEQ ID NO:992), wherein said first amino acid sequence and second amino acid sequence are contiguous and in a sequential order.

2. An isolated polypeptide encoding for a head of R30650_PEA_{—}2_P5 (SEQ ID NO:992), comprising a polypeptide being at least 70%, optionally at least about 80%, preferably at least about 85%, more preferably at least about 90% and most preferably at least about 95% homologous to the sequence MDGVNLSTEVVYKKGQDYRFACYDRGRACRSYRVRFLCGKPVRPKLTVTIDTNVNSTILNLEDNVQSWK PGDTLVIASTDYSMYQAEEFQVLPCRSCAPNQVKVAGKPMYLHIGEEIDGVDMRAEVGLLSRNIIVMGEM EDKCYPYRNHICNFFDFDTFGGHIKFALGFKAAHLEGTELKHMGQQLVGQYPIHFHLAGDVDERGGYDPP TYIRDLSIHHTFSRCVTVHGSNGLLIKDVVGYNSLGHCFFTEDGPEERNTFDHCLGLLVKSGTLLPSDRDSK MCKMITEDSYPGYIPKPRQDCNAVSTFWMANPNNNLINCAAAGSEETGFWFIFHHVPTGPSVGMYSPGYS EHIPLGKFYNNRAHSNYRAGMIIDNGVKTTEASAKDKRPFLSIISARYSPHQDADPLKPREPAIIRHFIAYKN QDHGAWLRGGDVWLDSCRFADNGIGLTLASGGTFPYDDGSKQEIKNSLFVGESGNVGTEMMDNRIWGP GGLDH of R30650_PEA_{—}2_P5 (SEQ ID NO:992).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein R30650_PEA_{—}2_P5 (SEQ ID NO:992) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein R30650_PEA_{—}2_P5 (SEQ ID NO:992) sequence provides support for the deduced sequence of this variant protein according to the present invention).

TABLE 10
Amino acid mutations

| SNP position(s) on amino acid | | Previously |
|---|---|---|
| sequence | Alternative amino acid(s) | known SNP? |
| 372 | M -> V | Yes |
| 418 | H -> R | Yes |

Variant protein R30650_PEA_{—}2_P5 (SEQ ID NO:992) is encoded by the following transcript(s): R30650_PEA_{—}2_T3 (SEQ ID NO:930), for which the sequence(s) is/are given at the end of the application. The coding portion of transcript R30650_PEA_{—}2_T3 (SEQ ID NO:930) is shown in bold; this coding portion starts at position 532 and ends at position 3519. The transcript also has the following SNPs as listed in Table 11 (given according to their position on the nucleotide sequence, with the alternative nucleic acid listed; the last column indicates whether the SNP is known or not; the presence of known SNPs in variant protein R30650_PEA_{—}2_P5 (SEQ ID NO:992) sequence provides support for the deduced sequence of this variant protein according to the present invention).

TABLE 11
Nucleic acid SNPs

| SNP position on nucleotide | | Previously |
|---|---|---|
| sequence | Alternative nucleic acid | known SNP? |
| 15 | A -> G | Yes |
| 112 | T -> C | Yes |
| 1320 | T -> C | Yes |
| 1407 | A -> G | Yes |
| 1645 | A -> G | Yes |
| 1784 | A -> G | Yes |
| 3667 | G -> C | Yes |
| 3883 | G -> A | Yes |
| 4944 | G -> A | Yes |
| 5992 | C -> T | Yes |
| 6131 | T -> C | Yes |
| 6223 | A -> C | Yes |

Variant protein R30650_PEA_{—}2_P8 (SEQ ID NO:993) according to the present invention has an amino acid sequence as given at the end of the application; it is encoded by transcript(s) R30650_PEA_{—}2_T6 (SEQ ID NO:931). An alignment is given to the known protein (Protein KIAA1199 precursor (SEQ ID NO:986)) at the end of the application. One or more alignments to one or more previously published protein sequences are given at the end of the application. A brief description of the relationship of the variant protein according to the present invention to each such aligned protein is as follows:

Comparison Report Between R30650_PEA_{—}2_P8 (SEQ ID NO:993) and Q9ULM1 (SEQ ID NO:989):

1. An isolated chimeric polypeptide encoding for R30650_PEA_{—}2_P8 (SEQ ID NO:993), comprising a first amino acid sequence being at least 70%, optionally at least 80%, preferably at least 85%, more preferably at least 90% and most preferably at least 95% homologous to a polypeptide having the sequence MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWK corresponding to amino acids 1-348 of R30650_PEA_{—}2_P8 (SEQ ID NO:993), a second amino acid sequence being at least 90% homologous to AHPGKICNRPIDIQATTMDGVNLSTEVVYKKGQDYRFACYDRGRACRSYRVRFLCGKPVRPKLTVTIDTN VNSTILNLEDNVQSWKPGDTLVIASTDYSMYQAEEFQVLPCRSCAPNQVKVAGKPMYLHIGEEIDGVDMR AEVGLLSRNIIVMGEMEDKCYPYRNHICNFFDFDTFGGHIKFALGFKAAHLEGTELKHMGQQLVGQYPIH FHLAGDVDERGGYDPPTYIRDLSIHHTFSRCVTVHGSNGLLIKDVVGYNSLGHCFFTEDGPEERNTFDHCL GLLVKSGTLLPSDRDSKMCKMITEDSYPGYIPKPRQDCNAVSTFWMANPNNNLINCAAAGSEETGFWFIF HHVPTGPSVGMYSPGYSEHIPLGKFYNNRAHSNYRAGMIIDNGVKTTEASAKDKRPFLSIISARYSPHQDA DPLKPREPAIIRHFIAYKNQDHGAWLRGGDVWLDSCRFADNGIGLTLASGGTFPYDDGSKQEIKNSLFVGE SGNVGTEMMDNRIWGPGGLDHSGRTLPIGQNFPIRGIQLYDGPINIQNCTFRKFVALEGRHTSALAFRLNN AWQSCPHNNVTGIAFEDVPITSRVFFGEPGPWFNQLDMDGDKTSVFHDVDGSVSEYPGSYLTKNDNWLV RHPDCINVPDWRGAICSGCYAQMYIQAYKTSNLRMKIIKNDFPSHPLYLEGALTRSTHYQQYQPVVTLQK GYTIHWDQTAPAELAIWLINFNKGDWIRVGLCYPRGTTFSILSDVHNRLLKQTSKTGVFVRTLQMDKVEQ SYPGRSHYYWDEDSG corresponding to amino acids 1-788 of Q9ULM1 (SEQ ID NO:989), which also corresponds to amino acids 349-1136 of R30650_PEA_{—}2_P8 (SEQ ID NO:993), and a third amino acid sequence being at least 70%, optionally at least 80%, preferably at least 85%, more preferably at least 90% and most preferably at least 95% homologous to a polypeptide having the sequence KQRTISWR (SEQ ID NO:1470) corresponding to amino acids 1137-1144 of R30650_PEA_{—}2_P8 (SEQ ID NO:993), wherein said first amino acid sequence, second amino acid sequence and third amino acid sequence are contiguous and in a sequential order.

2. An isolated polypeptide encoding for a head of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWK of R30650_PEA_{—}2_P8 (SEQ ID NO:993).

3. An isolated polypeptide encoding for a tail of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence KQRTISWR (SEQ ID NO:1470) in R30650_PEA_{—}2_P8 (SEQ ID NO:993).

Comparison Report Between R30650_PEA_{—}2_P8 (SEQ ID NO:993) and Q8WUJ3:

1. An isolated chimeric polypeptide encoding for R30650_PEA_{—}2_P8 (SEQ ID NO:993), comprising a first amino acid sequence being at least 90% homologous to MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWKAHP GKICNRPIDIQATTMDGVNLSTEVVYKKGQDYRFACYDRGRACRSYRVRFLCGKPVRPKLTVTIDTNVNS TILNLEDNVQSWKPGDTLVIASTDYSMYQAEEFQVLPCRSCAPNQVKVAGKPMYLHIGEEIDGVDMRAEV GLLSRNIIVMGEMEDKCYPYRNHICNFFDFDTFGGHIKFALGFKAAHLEGTELKHMGQQLVGQYPIHFHL AGDVDERGGYDPPTYIRDLSIHHTFSRCVTVHGSNGLLIKDVVGYNSLGHCFFTEDGPEERNTFDHCLGLL VKSGTLLPSDRDSKMCKMITEDSYPGYIPKPRQDCNAVSTFWMANPNNNLINCAAAGSEETGFWFIFHHV PTGPSVGMYSPGYSEHIPLGKFYNNRAHSNYRAGMIIDNGVKTTEASAKDKRPFLSIISARYSPHQDADPLK PREPAIIRHFIAYKNQDHGAWLRGGDVWLDSCRFADNGIGLTLASGGTFPYDDGSKQEIKNSLFVGESGNV GTEMMDNRIWGPGGLDHSGRTLPIGQNFPIRGIQLYDGPINIQNCTFRKFVALEGRHTSALAFRLNNAWQS CPHNNVTGIAFEDVPITSRVFFGEPGPWFNQLDMDGDKTSVFHDVDGSVSEYPGSYLTKND corresponding to amino acids 1-977 of Q8WUJ3 (SEQ ID NO:987), which also corresponds to amino acids 1-977 of R30650_PEA_{—}2_P8 (SEQ ID NO:993), and a second amino acid sequence being at least 70%, optionally at least 80%, preferably at least 85%, more preferably at least 90% and most preferably at least 95% homologous to a polypeptide having the sequence NWLVRHPDCINVPDWRGAICSGCYAQMYIQAYKTSNLRMKIIKNDFPSHPLYLEGALTRSTHYQQYQPVV TLQKGYTIHWDQTAPAELAIWLINFNKGDWIRVGLCYPRGTTFSILSDVHNRLLKQTSKTGVFVRTLQMD KVEQSYPGRSHYYWDEDSGKQRTISWR corresponding to amino acids 978-1144 of R30650_PEA_{—}2_P8 (SEQ ID NO:993), wherein said first amino acid sequence and second amino acid sequence are contiguous and in a sequential order.

2. An isolated polypeptide encoding for a tail of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence NWLVRHPDCINVPDWRGAICSGCYAQMYIQAYKTSNLRMKIIKNDFPSHPLYLEGALTRSTHYQQYQPVV TLQKGYTIHWDQTAPAELAIWLINFNKGDWIRVGLCYPRGTTFSILSDVHNRLLKQTSKTGVFVRTLQMD KVEQSYPGRSHYYWDEDSGKQRTISWR in R30650_PEA_{—}2_P8 (SEQ ID NO:993).

Comparison Report Between R30650_PEA_{—}2_P8 (SEQ ID NO:993) and Q9NPN9:

1. An isolated chimeric polypeptide encoding for R30650_PEA_{—}2_P8 (SEQ ID NO:993), comprising a first amino acid sequence being at least 70%, optionally at least 80%, preferably at least 85%, more preferably at least 90% and most preferably at least 95% homologous to a polypeptide having the sequence MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWKAHP GKICNRPIDIQATTMDGVNLSTEVVYKKGQDYRFACYDRGRACRSYRVRFLCGKPVRPKLTVTIDTNVNS TILNLEDNVQSWKPGDTLVIASTDYSMYQAEEFQVLPCRSCAPNQVKVAGKPMYLHIGEEIDGVDMRAEV GLLSRNIIVMGEMEDKCYPYRNHICNFFDFDTFGGHIKFALGFKAAHLEGTELKHMGQQLVGQYPIHFHL AGD corresponding to amino acids 1-564 of R30650_PEA_{—}2_P8 (SEQ ID NO:993), a second amino acid sequence being at least 90% homologous to VDERGGYDPPTYIRDLSIHHTFSRCVTVHGSNGLLIKDVVGYNSLGHCFFTEDGPEERNTFDHCLGLLVKS GTLLPSDRDSKMCKMITEDSYPGYIPKPRQDCNAVSTFWMANPNNNLINCAAAGSEETGFWFIFHHVPTG PSVGMYSPGYSEHIPLGKFYNNRAHSNYRAGMIIDNGVKTTEASAKDKRPFLSIISARYSPHQDADPLKPRE PAIIRHFIAYKNQDHGAWLRGGDVWLDSCRFADNGIGLTLASGGTFPYDDGSKQEIKNSLFVGESGNVGT EMMDNRIWGPGGLDHSGRTLPIGQNFPIRGIQLYDGPINIQNCTFRKFVALEGRHTSALAFRLNNAWQSCP HNNVTGIAFEDVPITSRVFFGEPGPWFNQLDMDGDKTSVFHDVDGSVSEYPGSYLTKNDNWLVRHPDCIN VPDWRGAICSGCYAQMYIQAYKTSNLRMKIIKNDFPSHPLYLEGALTRSTHYQQYQPVVTLQKGYTIHWD QTAPAELAIWLINFNKGDWIRVGLCYPRGTTFSILSDVHNRLLKQTSKTGVFVRTLQMDKVEQSYPGRSH YYWDEDSG corresponding to amino acids 8-579 of Q9NPN9 (SEQ ID NO:988), which also corresponds to amino acids 565-1136 of R30650_PEA_{—}2_P8 (SEQ ID NO:993), and a third amino acid sequence being at least 70%, optionally at least 80%, preferably at least 85%, more preferably at least 90% and most preferably at least 95% homologous to a polypeptide having the sequence KQRTISWR (SEQ ID NO:1470) corresponding to amino acids 1137-1144 of R30650_PEA_{—}2_P8 (SEQ ID NO:993), wherein said first amino acid sequence, second amino acid sequence and third amino acid sequence are contiguous and in a sequential order.

2. An isolated polypeptide encoding for a head of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWKAHP GKICNRPIDIQATTMDGVNLSTEVVYKKGQDYRFACYDRGRACRSYRVRFLCGKPVRPKLTVTIDTNVNS TILNLEDNVQSWKPGDTLVIASTDYSMYQAEEFQVLPCRSCAPNQVKVAGKPMYLHIGEEIDGVDMRAEV GLLSRNIIVMGEMEDKCYPYRNHICNFFDFDTFGGHIKFALGFKAAHLEGTELKHMGQQLVGQYPIHFHL AGD of R30650_PEA_{—}2_P8 (SEQ ID NO:993).

3. An isolated polypeptide encoding for a tail of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence KQRTISWR (SEQ ID NO:1470) in R30650_PEA_{—}2_P8 (SEQ ID NO:993).

Comparison Report Between R30650_PEA_{—}2_P8 (SEQ ID NO:993) and Q9H1K5:

1. An isolated chimeric polypeptide encoding for R30650_PEA_{—}2_P8 (SEQ ID NO:993), comprising a first amino acid sequence being at least 70%, optionally at least 80%, preferably at least 85%, more preferably at least 90% and most preferably at least 95% homologous to a polypeptide having the sequence MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWKAHP GKICNRPIDIQATTMDGVNLSTEVVYKKGQDYRFACYDRGRACRSYRVRFLCGKPVRPKLTVTIDTNVNS TILNLEDNVQSWKPGDTLVIASTDYSMYQAEEFQVLPCRSCAPNQVKVAGKPMYLHIGEEIDGVDMRAEV GLLSRNIIVMGEMEDKCYPYRNHICNFFDFDTFGGHIKFALGFKAAHLEGTELKHMGQQLVGQYPIHFHL AGDVDERGGYDPPTYIRDLSIHHTFSRCVTVHGSNGLLIKDVVGYNSLGHCFFTEDGPEERNTFDHCLGLL VKSGTLLPSDRDSKMCKMITEDSYPGYIPKPRQDCNAVSTFWMANPNNNLINCAAAGSEETGFWFIFHHV PTGPSVGMYSPGYSEHIPLGKFYNNRAHSNYRAGMIIDNGVKTTEASAKDKRPFLSIISARYSPHQDADPLK PREPAIIRHFIAYKNQDHGAWLRGGDVWLDSCRFADNGIGLTLASGGTFPYDDGSKQEIKNSLFVGESGNV GTEMMDNRIWGPGGLDH corresponding to amino acids 1-862 of R30650_PEA_{—}2_P8 (SEQ ID NO:993), a second amino acid sequence being at least 90% homologous to SGRTLPIGQNFPIRGIQLYDGPINIQNCTFRKFVALEGRHTSALAFRLNNAWQSCPHNNVTGIAFEDVPITSR VFFGEPGPWFNQLDMDGDKTSVFHDVDGSVSEYPGSYLTKNDNWLVRHPDCINVPDWRGAICSGCYAQ MYIQAYKTSNLRMKIIKNDFPSHPLYLEGALTRSTHYQQYQPVVTLQKGYTIHWDQTAPAELAIWLINFNK GDWIRVGLCYPRGTTFSILSDVHNRLLKQTSKTGVFVRTLQMDKVEQSYPGRSHYYWDEDSG corresponding to amino acids 2-275 of Q9H1K5 (SEQ ID NO:990), which also corresponds to amino acids 863-1136 of R30650_PEA_{—}2_P8 (SEQ ID NO:993), and a third amino acid sequence being at least 70%, optionally at least 80%, preferably at least 85%, more preferably at least 90% and most preferably at least 95% homologous to a polypeptide having the sequence KQRTISWR (SEQ ID NO:1470) corresponding to amino acids 1137-1144 of R30650_PEA_{—}2_P8 (SEQ ID NO:993), wherein said first amino acid sequence, second amino acid sequence and third amino acid sequence are contiguous and in a sequential order.

2. An isolated polypeptide encoding for a head of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWKAHP GKICNRPIDIQATTMDGVNLSTEVVYKKGQDYRFACYDRGRACRSYRVRFLCGKPVRPKLTVTIDTNVNS TILNLEDNVQSWKPGDTLVIASTDYSMYQAEEFQVLPCRSCAPNQVKVAGKPMYLHIGEEIDGVDMRAEV GLLSRNIIVMGEMEDKCYPYRNHICNFFDFDTFGGHIKFALGFKAAHLEGTELKHMGQQLVGQYPIHFHL AGDVDERGGYDPPTYIRDLSIHHTFSRCVTVHGSNGLLIKDVVGYNSLGHCFFTEDGPEERNTFDHCLGLL VKSGTLLPSDRDSKMCKMITEDSYPGYIPKPRQDCNAVSTFWMANPNNNLINCAAAGSEETGFWFIFHHV PTGPSVGMYSPGYSEHIPLGKFYNNRAHSNYRAGMIIDNGVKTTEASAKDKRPFLSIISARYSPHQDADPLK PREPAIIRHFIAYKNQDHGAWLRGGDVWLDSCRFADNGIGLTLASGGTFPYDDGSKQEIKNSLFVGESGNV GTEMMDNRIWGPGGLDH of R30650_PEA_{—}2_P8 (SEQ ID NO:993).

3. An isolated polypeptide encoding for a tail of R30650_PEA_{—}2_P8 (SEQ ID NO:993), comprising a polypeptide being at least 70%, optionally at least about 80%, preferably at least about 85%, more preferably at least about 90% and most preferably at least about 95% homologous to the sequence KQRTISWR (SEQ ID NO:1470) in R30650_PEA_{—}2_P8 (SEQ ID NO:993).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein R30650_PEA_{—}2_P8 (SEQ ID NO:993) also has the following non-silent SNPs (Single Nucleotide Polymorphisms) as listed in Table 12, (given according to their position(s) on the amino acid sequence, with the alternative amino acid(s) listed; the last column indicates whether the SNP is known or not; the presence of known SNPs in variant protein R30650_PEA_{—}2_P8 (SEQ ID NO:993) sequence provides support for the deduced sequence of this variant protein according to the present invention).

TABLE 12
Amino acid mutations

| SNP position(s) on amino acid | | Previously |
|---|---|---|
| sequence | Alternative amino acid(s) | known SNP? |
| 737 | M -> V | Yes |
| 783 | H -> R | Yes |

Variant protein R30650_PEA_{—}2_P8 (SEQ ID NO:993) is encoded by the following transcript(s): R30650_PEA_{—}2_T6 (SEQ ID NO:931), for which the sequence(s) is/are given at the end of the application. The coding portion of transcript R30650_PEA_{—}2_T6 (SEQ ID NO:931) is shown in bold; this coding portion starts at position 265 and ends at position 3696. The transcript also has the following SNPs as listed in Table 13 (given according to their position on the nucleotide sequence, with the alternative nucleic acid listed; the last column indicates whether the SNP is known or not; the presence of known SNPs in variant protein R30650_PEA_{—}2_P8 (SEQ ID NO:993) sequence provides support for the deduced sequence of this variant protein according to the present invention).

TABLE 13
Nucleic acid SNPs

| SNP position on nucleotide | | Previously |
|---|---|---|
| sequence | Alternative nucleic acid | known SNP? |
| 9 | T -> A | Yes |
| 2148 | T -> C | Yes |
| 2235 | A -> G | Yes |
| 2473 | A -> G | Yes |
| 2612 | A -> G | Yes |
| 4290 | G -> C | Yes |
| 4506 | G -> A | Yes |
| 5567 | G -> A | Yes |
| 6615 | C -> T | Yes |
| 6754 | T -> C | Yes |
| 6846 | A -> C | Yes |

Variant protein R30650_PEA_{—}2_P12 (SEQ ID NO:994) according to the present invention has an amino acid sequence as given at the end of the application; it is encoded by transcript(s) R30650_PEA_{—}2_T14 (SEQ ID NO:932). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein R30650_PEA_{—}2_P12 (SEQ ID NO:994) is encoded by the following transcript(s): R30650_PEA_{—}2_T14 (SEQ ID NO:932), for which the sequence(s) is/are given at the end of the application. The coding portion of transcript R30650_PEA_{—}2_T14 (SEQ ID NO:932) is shown in bold; this coding portion starts at position 1543 and ends at position 1719. The transcript also has the following SNPs as listed in Table 14 (given according to their position on the nucleotide sequence, with the alternative nucleic acid listed; the last column indicates whether the SNP is known or not; the presence of known SNPs in variant protein R30650_PEA_{—}2_P12 (SEQ ID NO:994) sequence provides support for the deduced sequence of this variant protein according to the present invention).

TABLE 14
Nucleic acid SNPs

| SNP position on nucleotide | | Previously |
|---|---|---|
| sequence | Alternative nucleic acid | known SNP? |
| 9 | T -> A | Yes |
| 381 | C -> T | Yes |
| 788 | C -> G | Yes |
| 799 | A -> G | No |
| 1324 | A -> T | Yes |
| 1437 | T -> C | Yes |
| 1441 | G -> A | Yes |
| 1513 | T -> C | Yes |
| 1529 | A -> G | Yes |
| 2087 | C -> A | Yes |
| 2182 | C -> T | Yes |

Variant protein R30650_PEA_{—}2_P13 (SEQ ID NO:995) according to the present invention has an amino acid sequence as given at the end of the application; it is encoded by transcript(s) R30650_PEA_{—}2_T15 (SEQ ID NO:933) and R30650_PEA_{—}2_T21 (SEQ ID NO:935). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein R30650_PEA_{—}2_P13 (SEQ ID NO:995) is encoded by the following transcript(s): R30650_PEA_{—}2_T15 (SEQ ID NO:933) and R30650_PEA_{—}2_T21 (SEQ ID NO:935), for which the sequence(s) is/are given at the end of the application.

The coding portion of transcript R30650_PEA_{—}2_T15 (SEQ ID NO:933) is shown in bold; this coding portion starts at position 1543 and ends at position 1713. The transcript also has the following SNPs as listed in Table 15 (given according to their position on the nucleotide sequence, with the alternative nucleic acid listed; the last column indicates whether the SNP is known or not; the presence of known SNPs in variant protein R30650_PEA_{—}2_P13 (SEQ ID NO:995) sequence provides support for the deduced sequence of this variant protein according to the present invention).

TABLE 15
Nucleic acid SNPs

| SNP position on nucleotide | | Previously |
|---|---|---|
| sequence | Alternative nucleic acid | known SNP? |
| 9 | T -> A | Yes |
| 381 | C -> T | Yes |
| 788 | C -> G | Yes |
| 799 | A -> G | No |
| 1324 | A -> T | Yes |
| 1437 | T -> C | Yes |
| 1441 | G -> A | Yes |
| 1513 | T -> C | Yes |
| 1529 | A -> G | Yes |
| 1920 | C -> A | Yes |
| 2015 | C -> T | Yes |

The coding portion of transcript R30650_PEA_{—}2_T21 (SEQ ID NO:935) is shown in bold; this coding portion starts at position 1543 and ends at position 1713. The transcript also has the following SNPs as listed in Table 16 (given according to their position on the nucleotide sequence, with the alternative nucleic acid listed; the last column indicates whether the SNP is known or not; the presence of known SNPs in variant protein R30650_PEA_{—}2_P13 (SEQ ID NO:995) sequence provides support for the deduced sequence of this variant protein according to the present invention).

TABLE 16
Nucleic acid SNPs

| SNP position on nucleotide | | Previously |
|---|---|---|
| sequence | Alternative nucleic acid | known SNP? |
| 9 | T -> A | Yes |
| 381 | C -> T | Yes |
| 788 | C -> G | Yes |
| 799 | A -> G | No |
| 1324 | A -> T | Yes |
| 1437 | T -> C | Yes |
| 1441 | G -> A | Yes |
| 1513 | T -> C | Yes |
| 1529 | A -> G | Yes |
| 1956 | T -> C | Yes |

Variant protein R30650_PEA_{—}2_P15 (SEQ ID NO:996) according to the present invention has an amino acid sequence as given at the end of the application; it is encoded by transcript(s) R30650_PEA_{—}2_T18 (SEQ ID NO:934). An alignment is given to the known protein (Protein KIAA1199 precursor (SEQ ID NO:986)) at the end of the application. One or more alignments to one or more previously published protein sequences are given at the end of the application. A brief description of the relationship of the variant protein according to the present invention to each such aligned protein is as follows:

Comparison Report Between R30650_PEA_{—}2_P15 (SEQ ID NO:996) and Q9ULM1 (SEQ ID NO:989):

1. An isolated chimeric polypeptide encoding for R30650_PEA_{—}2_P15 (SEQ ID NO:996), comprising a first amino acid sequence being at least 70%, optionally at least 80%, preferably at least 85%, more preferably at least 90% and most preferably at least 95% homologous to a polypeptide having the sequence MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWK corresponding to amino acids 1-348 of R30650_PEA_{—}2_P15 (SEQ ID NO:996), and a second amino acid sequence being at least 90% homologous to AHPGKICNRPIDIQATTMDGVNLSTEVVYKKGQDYRFACYDRGRACRSYRVRFLCGKPVRPKLTVTIDTN VNSTILNLEDNVQSWKPGDTLVIASTDYSMYQAEEFQVLPCRSCAPNQVKVAGKPMYLHIGEEIDGVDMR AEVGLLSRNIIVMGEMEDKCYPYRNHICNFFDFDTFGGHIKFALGFKAAHLEGTELKHMGQQLVGQYPIH FHLAGDVDERGGYDPPTYIRDLSIHHTFSRCVTVHGSNGLLIKDVVGYNSLGHCFFTEDGPEERNTFDHCL GLLVKSGTLLPSDRDSKMCKMITEDSYPGYIPKPRQDCNAVSTFWMANPNNNLINCAAAGSEETGFWFIF HHVPTGPSVGMYSPGYSEHIPLGKFYNNRAHSNYRAGMIIDNGVKTTEASAKDKRPFLSIISARYSPHQDA DPLKPREPAIIRHFIAYKNQDHGAWLRGGDVWLDSCRFADNGIGLTLASGGTFPYDDGSKQEIKNSLFVGE SGNVGTEMMDNRIWGPGGLDHSGRTLPIGQNFPIRGIQLYDGPINIQNCTFRKFVALEGRHTSALAFRLNN AWQSCPHNNVTGIAFEDVPITSRVFFGEPGPWFNQLDMDGDKTSVFHDVDGSVSEYPGSYLTKNDNWLV RHPDCINVPDWRGAICSGCYAQMYIQAYKTSNLRMKIIKNDFPSHPLYLEGALTRSTHYQQYQPVVTLQK GYTIHWDQTAPAELAIWLINFNKGDWIRVGLCYPRGTTFSILSDVHNRLLKQTSKTGVFVRTLQMDKVEQ SYPGRSHYYWDEDSG corresponding to amino acids 1-788 of Q9ULM1 (SEQ ID NO:989), which also corresponds to amino acids 349-1136 of R30650_PEA_{—}2_P15 (SEQ ID NO:996), wherein said first amino acid sequence and second amino acid sequence are contiguous and in a sequential order.

2. An isolated polypeptide encoding for a head of R30650_PEA_{—}2_P15 (SEQ ID NO:996), comprising a polypeptide being at least 70%, optionally at least about 80%, preferably at least about 85%, more preferably at least about 90% and most preferably at least about 95% homologous to the sequence MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWK of R30650_PEA_{—}2_P15 (SEQ ID NO:996).

Comparison Report Between R30650_PEA_{—}2_P15 (SEQ ID NO:996) and Q8WUJ3 (SEQ ID NO:987):

1. An isolated chimeric polypeptide encoding for R30650_PEA_{—}2_P15 (SEQ ID NO:996), comprising a first amino acid sequence being at least 90% homologous to MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWKAHP GKICNRPIDIQATTMDGVNLSTEVVYKKGQDYRFACYDRGRACRSYRVRFLCGKPVRPKLTVTIDTNVNS TILNLEDNVQSWKPGDTLVIASTDYSMYQAEEFQVLPCRSCAPNQVKVAGKPMYLHIGEEIDGVDMRAEV GLLSRNIIVMGEMEDKCYPYRNHICNFFDFDTFGGHIKFALGFKAAHLEGTELKHMGQQLVGQYPIHFHL AGDVDERGGYDPPTYIRDLSIHHTFSRCVTVHGSNGLLIKDVVGYNSLGHCFFTEDGPEERNTFDHCLGLL VKSGTLLPSDRDSKMCKMITEDSYPGYIPKPRQDCNAVSTFWMANPNNNLINCAAAGSEETGFWFIFHHV PTGPSVGMYSPGYSEHIPLGKFYNNRAHSNYRAGMIIDNGVKTTEASAKDKRPFLSIISARYSPHQDADPLK PREPAIIRHFIAYKNQDHGAWLRGGDVWLDSCRFADNGIGLTLASGGTFPYDDGSKQEIKNSLFVGESGNV GTEMMDNRIWGPGGLDHSGRTLPIGQNFPIRGIQLYDGPINIQNCTFRKFVALEGRHTSALAFRLNNAWQS CPHNNVTGIAFEDVPITSRVFFGEPGPWFNQLDMDGDKTSVFHDVDGSVSEYPGSYLTKND corresponding to amino acids 1-977 of Q8WUJ3 (SEQ ID NO:987), which also corresponds to amino acids 1-977 of R30650_PEA_{—}2_P15 (SEQ ID NO:996), and a second amino acid sequence being at least 70%, optionally at least 80%, preferably at least 85%, more preferably at least 90% and most preferably at least 95% homologous to a polypeptide having the sequence NWLVRHPDCINVPDWRGAICSGCYAQMYIQAYKTSNLRMKIIKNDFPSHPLYLEGALTRSTHYQQYQPVV TLQKGYTIHWDQTAPAELAIWLINFNKGDWIRVGLCYPRGTTFSILSDVHNRLLKQTSKTGVFVRTLQMD KVEQSYPGRSHYYWDEDSG corresponding to amino acids 978-1136 of R30650_PEA_{—}2_P15 (SEQ ID NO:996), wherein said first amino acid sequence and second amino acid sequence are contiguous and in a sequential order.

2. An isolated polypeptide encoding for a tail of R30650_PEA_{—}2_P15 (SEQ ID NO:996), comprising a polypeptide being at least 70%, optionally at least about 80%, preferably at least about 85%, more preferably at least about 90% and most preferably at least about 95% homologous to the sequence NWLVRHPDCINVPDWRGAICSGCYAQMYIQAYKTSNLRMKIIKNDFPSHPLYLEGALTRSTHYQQYQPVV TLQKGYTIHWDQTAPAELAIWLINFNKGDWIRVGLCYPRGTTFSILSDVHNRLLKQTSKTGVFVRTLQMD KVEQSYPGRSHYYWDEDSG in R30650_PEA_{—}2_P15 (SEQ ID NO:996).

Comparison Report Between R30650_PEA_{—}2_P15 (SEQ ID NO:996) and Q9NPN9 (SEQ ID NO:988):

1. An isolated chimeric polypeptide encoding for R30650_PEA_{—}2_P15 (SEQ ID NO:996), comprising a first amino acid sequence being at least 70%, optionally at least 80%, preferably at least 85%, more preferably at least 90% and most preferably at least 95% homologous to a polypeptide having the sequence MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWKAHP GKICNRPIDIQATTMDGVNLSTEVVYKKGQDYRFACYDRGRACRSYRVRFLCGKPVRPKLTVTIDTNVNS TILNLEDNVQSWKPGDTLVIASTDYSMYQAEEFQVLPCRSCAPNQVKVAGKPMYLHIGEEIDGVDMRAEV GLLSRNIIVMGEMEDKCYPYRNHICNFFDFDTFGGHIKFALGFKAAHLEGTELKHMGQQLVGQYPIHFHL AGD corresponding to amino acids 1-564 of R30650_PEA_{—}2_P15 (SEQ ID NO:996), and a second amino acid sequence being at least 90% homologous to VDERGGYDPPTYIRDLSIHHTFSRCVTVHGSNGLLIKDVVGYNSLGHCFFTEDGPEERNTFDHCLGLLVKS GTLLPSDRDSKMCKMITEDSYPGYIPKPRQDCNAVSTFWMANPNNNLINCAAAGSEETGFWFIFHHVPTG PSVGMYSPGYSEHIPLGKFYNNPAHSNYRAGMIIDNGVKTTEASAKDKRPFLSIISARYSPHQDADPLKPRE PAIIRHFIAYKNQDHGAWLRGGDVWLDSCRFADNGIGLTLASGGTFPYDDGSKQEIKNSLFVGESGNVGT EMMDNRIWGPGGLDHSGRTLPIGQNFPIRGIQLYDGPINIQNCTFRKFVALEGRHTSALAFRLNNAWQSCP HNNVTGIAFEDVPITSRVFFGEPGPWFNQLDMDGDKTSVFHDVDGSVSEYPGSYLTKNDNWLVRHPDCIN VPDWRGAICSGCYAQMYIQAYKTSNLRMKIIKNDFPSHPLYLEGALTRSTHYQQYQPVVTLQKGYTIHWD QTAPAELAIWLINFNKGDWIRVGLCYPRGTTFSILSDVHNRLLKQTSKTGVFVRTLQMDKVEQSYPGRSH YYWDEDSG corresponding to amino acids 8-579 of Q9NPN9 (SEQ ID NO:988), which also corresponds to amino acids 565-1136 of R30650_PEA_{—}2_P15 (SEQ ID NO:996), wherein said first amino acid sequence and second amino acid sequence are contiguous and in a sequential order.

2. An isolated polypeptide encoding for a head of R30650_PEA_{—}2_P15 (SEQ ID NO:996), comprising a polypeptide being at least 70%, optionally at least about 80%, preferably at least about 85%, more preferably at least about 90% and most preferably at least about 95% homologous to the sequence MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWKAHP GKICNRPIDIQATTMDGVNLSTEVVYKKGQDYRFACYDRGRACRSYRVRFLCGKPVRPKLTVTIDTNVNS TILNLEDNVQSWKPGDTLVIASTDYSMYQAEEFQVLPCRSCAPNQVKVAGKPMYLHIGEEIDGVDMRAEV GLLSRNIIVMGEMEDKCYPYRNHICNFFDFDTFGGHIKFALGFKAAHLEGTELKHMGQQLVGQYPIHFHL AGD of R30650_PEA_{—}2_P15 (SEQ ID NO:996).

Comparison Report Between R30650_PEA_{—}2_P15 (SEQ ID NO:996) and Q9H1K5 (SEQ ID NO:990):

1. An isolated chimeric polypeptide encoding for R30650_PEA_{—}2_P15 (SEQ ID NO:996), comprising a first amino acid sequence being at least 70%, optionally at least 80%, preferably at least 85%, more preferably at least 90% and most preferably at least 95% homologous to a polypeptide having the sequence MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWKAHP GKICNRPIDIQATTMDGVNLSTEVVYKKGQDYRFACYDRGRACRSYRVRFLCGKPVRPKLTVTIDTNVNS TILNLEDNVQSWKPGDTLVIASTDYSMYQAEEFQVLPCRSCAPNQVKVAGKPMYLHIGEEIDGVDMRAEV GLLSRNIIVMGEMEDKCYPYRNHICNFFDFDTFGGHIKFALGFKAAHLEGTELKHMGQQLVGQYPIHFHL AGDVDERGGYDPPTYIRDLSIHHTFSRCVTVHGSNGLLIKDVVGYNSLGHCFFTEDGPEERNTFDHCLGLL VKSGTLLPSDRDSKMCKMITEDSYPGYIPKPRQDCNAVSTFWMANPNNNLINCAAAGSEETGFWFIFHHV PTGPSVGMYSPGYSEHIPLGKFYNNRAHSNYRAGMIIDNGVKTTEASAKDKRPFLSIISARYSPHQDADPLK PREPAIIRHFIAYKNQDHGAWLRGGDVWLDSCRFADNGIGLTLASGGTFPYDDGSKQEIKNSLFVGESGNV GTEMMDNRIWGPGGLDH corresponding to amino acids 1-862 of R30650_PEA_{—}2_P15 (SEQ ID NO:996), and a second amino acid sequence being at least 90% homologous to SGRTLPIGQNFPIRGIQLYDGPINIQNCTFRKFVALEGRHTSALAFRLNNAWQSCPHNNVTGIAFEDVPITSR VFFGEPGPWFNQLDMDGDKTSVFHDVDGSVSEYPGSYLTKNDNWLVRHPDCINVPDWRGAICSGCYAQ MYIQAYKTSNLRMKIIKNDFPSHPLYLEGALTRSTHYQQYQPVVTLQKGYTIHWDQTAPAELAIWLINFNK GDWIRVGLCYPRGTTFSILSDVHNRLLKQTSKTGVFVRTLQMDKVEQSYPGRSHYYWDEDSG corresponding to amino acids 2-275 of Q9H1K5 (SEQ ID NO:990), which also corresponds to amino acids 863-1136 of R30650_PEA_{—}2_P15 (SEQ ID NO:996), wherein said first amino acid sequence and second amino acid sequence are contiguous and in a sequential order.

2. An isolated polypeptide encoding for a head of R30650_PEA_{—}2_P15 (SEQ ID NO:996), comprising a polypeptide being at least 70%, optionally at least about 80%, preferably at least about 85%, more preferably at least about 90% and most preferably at least about 95% homologous to the sequence MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQDVEWTEWFDHDKVSQTKGGEKISDLWKAHP GKICNRPIDIQATTMDGVNLSTEVVYKKGQDYRFACYDRGRACRSYRVRFLCGKPVRPKLTVTIDTNVNS TILNLEDNVQSWKPGDTLVIASTDYSMYQAEEFQVLPCRSCAPNQVKVAGKPMYLHIGEEIDGVDMRAEV GLLSRNIIVMGEMEDKCYPYRNHICNFFDFDTFGGHIKFALGFKAAHLEGTELKHMGQQLVGQYPIHFHL AGDVDERGGYDPPTYIRDLSIHHTFSRCVTVHGSNGLLIKDVVGYNSLGHCFFTEDGPEERNTFDHCLGLL VKSGTLLPSDRDSKMCKMITEDSYPGYIPKPRQDCNAVSTFWMANPNNNLINCAAAGSEETGFWFIFHHV PTGPSVGMYSPGYSEHIPLGKFYNNRAHSNYRAGMIIDNGVKTTEASAKDKRPFLSIISARYSPHQDADPLK PREPAIIRHFIAYKNQDHGAWLRGGDVWLDSCRFADNGIGLTLASGGTFPYDDGSKQEIKNSLFVGESGNV GTEMMDNRIWGPGGLDH of R30650_PEA_{—}2_P15 (SEQ ID NO:996).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein R30650_PEA_{—}2_P15 (SEQ ID NO:996) also has the following non-silent SNPs (Single Nucleotide Polymorphisms) as listed in Table 17, (given according to their position(s) on the amino acid sequence, with the alternative amino acid(s) listed; the last column indicates whether the SNP is known or not; the presence of known SNPs in variant protein R30650_PEA_{—}2_P15 (SEQ ID NO:996) sequence provides support for the deduced sequence of this variant protein according to the present invention).

TABLE 17
Amino acid mutations

| SNP position(s) on amino acid | | Previously |
|---|---|---|
| sequence | Alternative amino acid(s) | known SNP? |
| 737 | M -> V | Yes |
| 783 | H -> R | Yes |

Variant protein R30650_PEA_{—}2_P15 (SEQ ID NO:996) is encoded by the following transcript(s): R30650_PEA_{—}2_T18 (SEQ ID NO:934), for which the sequence(s) is/are given at the end of the application. The coding portion of transcript R30650_PEA_{—}2_T18 (SEQ ID NO:934) is shown in bold; this coding portion starts at position 265 and ends at position 3672. The transcript also has the following SNPs as listed in Table 18 (given according to their position on the nucleotide sequence, with the alternative nucleic acid listed; the last column indicates whether the SNP is known or not; the presence of known SNPs in variant protein R30650_PEA_{—}2_P15 (SEQ ID NO:996) sequence provides support for the deduced sequence of this variant protein according to the present invention).

TABLE 18
Nucleic acid SNPs

| SNP position on nucleotide | | Previously |
|---|---|---|
| sequence | Alternative nucleic acid | known SNP? |
| 9 | T -> A | Yes |
| 2148 | T -> C | Yes |
| 2235 | A -> G | Yes |
| 2473 | A -> G | Yes |
| 2612 | A -> G | Yes |

Variant protein R30650_PEA_{—}2_P17 (SEQ ID NO:997) according to the present invention has an amino acid sequence as given at the end of the application; it is encoded by transcript(s) R30650_PEA_{—}2_T23 (SEQ ID NO:936). An alignment is given to the known protein (Protein KIAA1199 precursor (SEQ ID NO:986)) at the end of the application. One or more alignments to one or more previously published protein sequences are given at the end of the application. A brief description of the relationship of the variant protein according to the present invention to each such aligned protein is as follows:

Comparison Report Between R30650_PEA_{—}2_P17 (SEQ ID NO:997) and Q8WUJ3:

1. An isolated chimeric polypeptide encoding for R30650_PEA_{—}2_P17 (SEQ ID NO:997), comprising a first amino acid sequence being at least 90% homologous to MGAAGRQDFLFKAMLTISWLTLTCFPGATSTVAAGCPDQSPELQPWNPGHDQDHHVHIGQGKTLLLTSSA TVYSIHISEGGKLVIKDHDEPIVLRTRHILIDNGGELHAGSALCPFQGNFTIILYGRADEGIQPDPYYGLKYIG VGKGGALELHGQKKLSWTFLNKTLHPGGMAEGGYFFERSWGHRGVIVHVIDPKSGTVIHSDRFDTYRSK KESERLVQYLNAVPDGRILSVAVNDEGSRNLDDMARKAMTKLGSKHFLHLGFRHPWSFLTVKGNPSSSV EDHIEYHGHRGSAAARVFKLFQTEHGEYFNVSLSSEWVQ corresponding to amino acids 1-321 of Q8WUJ3 (SEQ ID NO:987), which also corresponds to amino acids 1-321 of R30650_PEA_{—}2_P17 (SEQ ID NO:997), and a second amino acid sequence being at least 70%, optionally at least 80%, preferably at least 85%, more preferably at least 90% and most preferably at least 95% homologous to a polypeptide having the sequence GEEFQTIW (SEQ ID NO:1473) corresponding to amino acids 322-329 of R30650_PEA_{—}2_P17 (SEQ ID NO:997), wherein said first amino acid sequence and second amino acid sequence are contiguous and in a sequential order.

2. An isolated polypeptide encoding for a tail of R30650_PEA_{—}2_P17 (SEQ ID NO:997), comprising a polypeptide being at least 70%, optionally at least about 80%, preferably at least about 85%, more preferably at least about 90% and most preferably at least about 95% homologous to the sequence GEEFQTIW (SEQ ID NO:1473) in R30650_PEA_{—}2_P17 (SEQ ID NO:997).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein R30650_PEA_{—}2_P17 (SEQ ID NO:997) is encoded by the following transcript(s): R30650_PEA_{—}2_T23 (SEQ ID NO:936), for which the sequence(s) is/are given at the end of the application. The coding portion of transcript R30650_PEA_{—}2_T23 (SEQ ID NO:936) is shown in bold; this coding portion starts at position 265 and ends at position 1251. The transcript also has the following SNPs as listed in Table 19 (given according to their position on the nucleotide sequence, with the alternative nucleic acid listed; the last column indicates whether the SNP is known or not; the presence of known SNPs in variant protein R30650_PEA_{—}2_P17 (SEQ ID NO:997) sequence provides support for the deduced sequence of this variant protein according to the present invention).

TABLE 19
Nucleic acid SNPs

| SNP position on nucleotide | | Previously |
|---|---|---|
| sequence | Alternative nucleic acid | known SNP? |
| 9 | T -> A | Yes |
| 1455 | T -> C | No |

As noted above, cluster R30650 features 49 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster R30650_PEA_{—}2_node_{—}0 (SEQ ID NO:937) according to the present invention is supported by 7 libraries. The number of libraries was determined as previously described. This segment can be found in the following transcript(s): R30650_PEA_{—}2_T6 (SEQ ID NO:931), R30650_PEA_{—}2_T14 (SEQ ID NO:932), R30650_PEA_{—}2_T15 (SEQ ID NO:933), R30650_PEA_{—}2_T18 (SEQ ID NO:934), R30650_PEA_{—}2_T21 (SEQ ID NO:935) and R30650_PEA_{—}2_T23 (SEQ ID NO:936). Table 20 below describes the starting and ending position of this segment on each transcript.

TABLE 20
Segment location on transcripts

| | Segment | |
|---|---|---|
| | starting | Segment |
| Transcript name | position | ending position |
| R30650_PEA_2_T6 (SEQ ID NO: 931) | 1 | 248 |
| R30650_PEA_2_T14 (SEQ ID NO: 932) | 1 | 248 |
| R30650_PEA_2_T15 (SEQ ID NO: 933) | 1 | 248 |
| R30650_PEA_2_T18 (SEQ ID NO: 934) | 1 | 248 |
| R30650_PEA_2_T21 (SEQ ID NO: 935) | 1 | 248 |
| R30650_PEA_2_T23 (SEQ ID NO: 936) | 1 | 248 |

Segment cluster R30650_PEA_{—}2_node_{—}1 (SEQ ID NO:938) according to the present invention is supported by 5 libraries. The number of libraries was determined as previously described. This segment can be found in the following transcript(s): R30650_PEA_{—}2_T14 (SEQ ID NO:932), R30650_PEA_{—}2_T15 (SEQ ID NO:933) and R30650_PEA_{—}2_T21 (SEQ ID NO:935). Table 21 below describes the starting and ending position of this segment on each transcript.

TABLE 21
Segment location on transcripts

| | Segment | |
|---|---|---|
| | starting | Segment |
| Transcript name | position | ending position |
| R30650_PEA_2_T14 (SEQ ID NO: 932) | 249 | 1237 |
| R30650_PEA_2_T15 (SEQ ID NO: 933) | 249 | 1237 |
| R30650_PEA_2_T21 (SEQ ID NO: 935) | 249 | 1237 |

Segment cluster R30650_PEA_{—}2_node_{—}3 (SEQ ID NO:939) according to the present invention is supported by 7 libraries. The number of libraries was determined as previously described. This segment can be found in the following transcript(s): R30650_PEA_{—}2_T14 (SEQ ID NO:932), R30650_PEA_{—}2_T15 (SEQ ID NO:933) and R30650_PEA_{—}2_T21 (SEQ ID NO:935). Table 22 below describes the starting and ending position of this segment on each transcript.

TABLE 22
Segment location on transcripts

| | Segment | |
|---|---|---|
| | starting | Segment |
| Transcript name | position | ending position |
| R30650_PEA_2_T14 (SEQ ID NO: 932) | 1238 | 1415 |
| R30650_PEA_2_T15 (SEQ ID NO: 933) | 1238 | 1415 |
| R30650_PEA_2_T21 (SEQ ID NO: 935) | 1238 | 1415 |

Segment cluster R30650_PEA_{—}2_node_{—}5 (SEQ ID NO:940) according to the present invention is supported by 8 libraries. The number of libraries was determined as previously described. This segment can be found in the following transcript(s): R30650_PEA_{—}2_T14 (SEQ ID NO:932), R30650_PEA_{—}2_T15 (SEQ ID NO:933) and R30650_PEA_{—}2_T21 (SEQ ID NO:935). Table 23 below describes the starting and ending position of this segment on each transcript.

TABLE 23
Segment location on transcripts

| | Segment | |
|---|---|---|
| | starting | Segment |
| Transcript name | position | ending position |
| R30650_PEA_2_T14 (SEQ ID NO: 932) | 1416 | 1617 |
| R30650_PEA_2_T15 (SEQ ID NO: 933) | 1416 | 1617 |
| R30650_PEA_2_T21 (SEQ ID NO: 935) | 1416 | 1617 |

Segment cluster R30650_PEA_{—}2_node_{—}9 (SEQ ID NO:941) according to the present invention is supported by 6 libraries. The number of libraries was determined as previously described. This segment can be found in the following transcript(s): R30650_PEA_{—}2_T21 (SEQ ID NO:935). Table 24 below describes the starting and ending position of this segment on each transcript.

TABLE 24
Segment location on transcripts

| | Segment | |
|---|---|---|
| | starting | Segment |
| Transcript name | position | ending position |
| R30650_PEA_2_T21 (SEQ ID NO: 935) | 1713 | 2152 |

Segment cluster R30650_PEA_{—}2_node_{—}11 (SEQ ID NO:942) according to the present invention is supported by 4 libraries. The number of libraries was determined as previously described. This segment can be found in the following transcript(s): R30650_PEA_{—}2_T14 (SEQ ID NO:932). Table 25 below describes the starting and ending position of this segment on each transcript.

TABLE 25
Segment location on transcripts

| | Segment | |
|---|---|---|
| | starting | Segment |
| Transcript name | position | ending position |
| R30650_PEA_2_T14 (SEQ ID NO: 932) | 1713 | 1879 |

Segment cluster R30650_PEA_{—}2_node_{—}14 (SEQ ID NO:943) according to the present invention is supported by 10 libraries. The number of libraries was determined as previously described. This segment can be found in the following transcript(s): R30650_PEA_{—}2_T14 (SEQ ID NO:932) and R30650_PEA_{—}2_T15 (SEQ ID NO:933). Table 26 below describes the starting and ending position of this segment on each transcript.

TABLE 26
Segment location on transcripts

| | Segment | |
|---|---|---|
| | starting | Segment |
| Transcript name | position | ending position |
| R30650_PEA_2_T14 (SEQ ID NO: 932) | 1880 | 2818 |
| R30650_PEA_2_T15 (SEQ ID NO: 933) | 1713 | 2651 |

Segment cluster R30650_PEA_{—}2_node_{—}20 (SEQ ID NO:944) according to the present invention is supported by 6 libraries. The number of libraries was determined as previously described. This segment can be found in the following transcript(s): R30650_PEA_{—}2_T6 (SEQ ID NO:931), R30650_PEA_{—}2_T18 (SEQ ID NO:934) and R30650_PEA_{—}2_T23 (SEQ ID NO:936). Table 27 below describes the starting and ending position of this segment on each transcript.

TABLE 27
Segment location on transcripts

| | Segment | |
|---|---|---|
| | starting | Segment |
| Transcript name | position | ending position |
| R30650_PEA_2_T6 (SEQ ID NO: 931) | 359 | 505 |
| R30650_PEA_2_T18 (SEQ ID NO: 934) | 359 | 505 |
| R30650_PEA_2_T23 (SEQ ID NO: 936) | 359 | 505 |

Segment cluster R30650_PEA_{—}2_node_{—}22 (SEQ ID NO:945) according to the present invention is supported by 6 libraries. The number of libraries was determined as previously described. This segment can be found in the following transcript(s): R30650_PEA_{—}2_T6 (SEQ ID NO:931), R30650_PEA_{—}2_T18 (SEQ ID NO:934) and R30650_PEA_{—}2_T23 (SEQ ID NO:936). Table 28 below describes the starting and ending position of this segment on each transcript.

TABLE 28
Segment location on transcripts

| | Segment | |
|---|---|---|
| | starting | Segment |
| Transcript name | position | ending position |
| R30650_PEA_2_T6 (SEQ ID NO: 931) | 506 | 644 |
| R30650_PEA_2_T18 (SEQ ID NO: 934) | 506 | 644 |
| R30650_PEA_2_T23 (SEQ ID NO: 936) | 506 | 644 |

Segment cluster R30650_PEA_{—}2_node_{—}24 (SEQ ID NO:946) according to the present invention is supported by 7 libraries. The number of libraries was determined as previously described. This segment can be found in the following transcript(s): R30650_PEA_{—}2_T6 (SEQ ID NO:931), R30650_PEA_{—}2_T18 (SEQ ID NO:934) and R30650_PEA_{—}2_T23 (SEQ ID NO:936). Table 29 below describes the starting and ending position of this segment on each transcript.

TABLE 29
Segment location on transcripts

| | Segment | |
|---|---|---|
| | starting | Segment |
| Transcript name | position | ending position |
| R30650_PEA_2_T6 (SEQ ID NO: 931) | 645 | 881 |
| R30650_PEA_2_T18 (SEQ ID NO: 934) | 645 | 881 |
| R30650_PEA_2_T23 (SEQ ID NO: 936) | 645 | 881 |

Segment cluster R30650_PEA_{—}2_node_{—}26 (SEQ ID NO:947) according to the present invention is supported by 7 libraries. The number of libraries was determined as previously described. This segment can be found in the following transcript(s): R30650_PEA_{—}2_T6 (SEQ ID NO:931), R30650_PEA_{—}2_T18 (SEQ ID NO:934) and R30650_PEA_{—}2_T23 (SEQ ID NO:936). Table 30 below describes the starting and ending position of this segment on each transcript.

TABLE 30
Segment location on transcripts

| | Segment | |
|---|---|---|
| | starting | Segment |
| Transcript name | position | ending position |
| R30650_PEA_2_T6 (SEQ ID NO: 931) | 882 | 1061 |
| R30650_PEA_2_T18 (SEQ ID NO: 934) | 882 | 1061 |
| R30650_PEA_2_T23 (SEQ ID NO: 936) | 882 | 1061 |

Segment cluster R30650_PEA_{—}2_node_{—}32 (SEQ ID NO:948) according to the present invention is supported by 4 libraries. The number of libraries was determined as previously described. This segment can be found in the following transcript(s): R30650_PEA_{—}2_T23 (SEQ ID NO:936). Table 31 below describes the starting and ending position of this segment on each transcript.

TABLE 31
Segment location on transcripts

| | Segment | |
|---|---|---|
| | starting | Segment |
| Transcript name | position | ending position |
| R30650_PEA_2_T23 (SEQ ID NO: 936) | 1229 | 1829 |

Segment cluster R30650_PEA_{—}2_node_{—}34 (SEQ ID NO:949) according to the present invention is supported by 8 libraries. The number of libraries was determined as previously described. This segment can be found in the following transcript(s): R30650_PEA_{—}2_T6 (SEQ ID NO:931) and R30650_PEA_{—}2_T18 (SEQ ID NO:934). Table 32 below describes the starting and ending position of this segment on each transcript.

TABLE 32
Segment location on transcripts

| | Segment | |
|---|---|---|
| | starting | Segment |
| Transcript name | position | ending position |
| R30650_PEA_2_T6 (SEQ ID NO: 931) | 1229 | 1350 |
| R30650_PEA_2_T18 (SEQ ID NO: 934) | 1229 | 1350 |

Segment cluster R30650_PEA_{—}2_node_{—}36 (SEQ ID NO:950) according to the present invention is supported by 3 libraries. The number of libraries was determined as previously described. This segment can be found in the following transcript(s): R30650_PEA_{—}2_T3 (SEQ ID NO:930). Table 33 below describes the starting and ending position of this segment on each transcript.

TABLE 33
Segment location on transcripts

| | Segment | |
|---|---|---|
| | starting | Segment |
| Transcript name | position | ending position |
| R30650_PEA_2_T3 (SEQ ID NO: 930) | 1 | 522 |

Segment cluster R30650_PEA_{—}2_node_{—}37 (SEQ ID NO:951) according to the present invention is supported by 8 libraries. The number of libraries was determined as previously described. This segment can be found in the following transcript(s): R30650_PEA_{—}2_T3 (SEQ ID NO:930), R30650_PEA_{—}2_T6 (SEQ ID NO:931) and R30650_PEA_{—}2_T18 (SEQ ID NO:934). Table 34 below describes the starting and ending position of this segment on each transcript.

TABLE 34
Segment location on transcripts

| | | Segment |
|---|---|---|
| | Segment | ending |
| Transcript name | starting position | position |
| R30650_PEA_2_T3 (SEQ ID NO: 930) | 523 | 655 |
| R30650_PEA_2_T6 (SEQ ID NO: 931) | 1351 | 1483 |
| R30650_PEA_2_T18 | 1351 | 1483 |
| (SEQ ID NO: 934) | | |

Segment cluster R30650_PEA_{—}2_node_{—}39 (SEQ ID NO:952) according to the present invention is supported by 8 libraries. The number of libraries was determined as previously described. This segment can be found in the following transcript(s): R30650_PEA_{—}2_T3 (SEQ ID NO:930), R30650_PEA_{—}2_T6 (SEQ ID NO:931) and R30650_PEA_{—}2_T18 (SEQ ID NO:934). Table 35 below describes the starting and ending position of this segment on each transcript.

TABLE 35
Segment location on transcripts

| | | Segment |
|---|---|---|
| | Segment | ending |
| Transcript name | starting position | position |
| R30650_PEA_2_T3 (SEQ ID NO: 930) | 656 | 847 |
| R30650_PEA_2_T6 (SEQ ID NO: 931) | 1484 | 1675 |
| R30650_PEA_2_T18 | 1484 | 1675 |
| (SEQ ID NO: 934) | | |

Segment cluster R30650_PEA_{—}2_node_{—}41 (SEQ ID NO:953) according to the present invention is supported by 3 libraries. The number of libraries was determined as previously described. This segment can be found in the following transcript(s): R30650_PEA_{—}2_T2 (SEQ ID NO:929). Table 36 below describes the starting and ending position of this segment on each transcript.

TABLE 36
Segment location on transcripts

| | | Segment |
|---|---|---|
| | Segment | ending |
| Transcript name | starting position | position |
| R30650_PEA_2_T2 (SEQ ID NO: 929) | 1 | 1360 |

Segment cluster R30650_PEA_{—}2_node_{—}42 (SEQ ID NO:954) according to the present invention is supported by 8 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 37 below describes the starting and ending position of this segment on each transcript.

TABLE 37
Segment location on transcripts

| | | Segment |
|---|---|---|
| | Segment | ending |
| Transcript name | starting position | position |
| R30650_PEA_2_T2 (SEQ ID NO: 929) | 1361 | 1536 |
| R30650_PEA_2_T3 (SEQ ID NO: 930) | 848 | 1023 |
| R30650_PEA_2_T6 (SEQ ID NO: 931) | 1676 | 1851 |
| R30650_PEA_2_T18 | 1676 | 1851 |
| (SEQ ID NO: 934) | | |

Segment cluster R30650_PEA_{—}2_node_{—}44 (SEQ ID NO:955) according to the present invention is supported by 8 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 38 below describes the starting and ending position of this segment on each transcript.

TABLE 38
Segment location on transcripts

| | | Segment |
|---|---|---|
| | Segment | ending |
| Transcript name | starting position | position |
| R30650_PEA_2_T2 (SEQ ID NO: 929) | 1537 | 1746 |
| R30650_PEA_2_T3 (SEQ ID NO: 930) | 1024 | 1233 |
| R30650_PEA_2_T6 (SEQ ID NO: 931) | 1852 | 2061 |
| R30650_PEA_2_T18 | 1852 | 2061 |
| (SEQ ID NO: 934) | | |

Segment cluster R30650_PEA_{—}2_node_{—}46 (SEQ ID NO:956) according to the present invention is supported by 9 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 39 below describes the starting and ending position of this segment on each transcript.

TABLE 39
Segment location on transcripts

| | | Segment |
|---|---|---|
| | Segment | ending |
| Transcript name | starting position | position |
| R30650_PEA_2_T2 (SEQ ID NO: 929) | 1747 | 1952 |
| R30650_PEA_2_T3 (SEQ ID NO: 930) | 1234 | 1439 |
| R30650_PEA_2_T6 (SEQ ID NO: 931) | 2062 | 2267 |
| R30650_PEA_2_T18 | 2062 | 2267 |
| (SEQ ID NO: 934) | | |

Segment cluster R30650_PEA_{—}2_node_{—}50 (SEQ ID NO:957) according to the present invention is supported by 9 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 40 below describes the starting and ending position of this segment on each transcript.

TABLE 40
Segment location on transcripts

| | | Segment |
|---|---|---|
| | Segment | ending |
| Transcript name | starting position | position |
| R30650_PEA_2_T2 (SEQ ID NO: 929) | 2023 | 2151 |
| R30650_PEA_2_T3 (SEQ ID NO: 930) | 1510 | 1638 |
| R30650_PEA_2_T6 (SEQ ID NO: 931) | 2338 | 2466 |
| R30650_PEA_2_T18 | 2338 | 2466 |
| (SEQ ID NO: 934) | | |

Segment cluster R30650_PEA_{—}2_node_{—}56 (SEQ ID NO:958) according to the present invention is supported by 10 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 41 below describes the starting and ending position of this segment on each transcript.

TABLE 41
Segment location on transcripts

| | | Segment |
|---|---|---|
| | Segment | ending |
| Transcript name | starting position | position |
| R30650_PEA_2_T2 (SEQ ID NO: 929) | 2238 | 2369 |
| R30650_PEA_2_T3 (SEQ ID NO: 930) | 1725 | 1856 |
| R30650_PEA_2_T6 (SEQ ID NO: 931) | 2553 | 2684 |
| R30650_PEA_2_T18 | 2553 | 2684 |
| (SEQ ID NO: 934) | | |

Segment cluster R30650_PEA_{—}2_node_{—}60 (SEQ ID NO:959) according to the present invention is supported by 12 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 42 below describes the starting and ending position of this segment on each transcript.

TABLE 42
Segment location on transcripts

| | | Segment |
|---|---|---|
| | Segment | ending |
| Transcript name | starting position | position |
| R30650_PEA_2_T2 (SEQ ID NO: 929) | 2406 | 2561 |
| R30650_PEA_2_T3 (SEQ ID NO: 930) | 1893 | 2048 |
| R30650_PEA_2_T6 (SEQ ID NO: 931) | 2721 | 2876 |
| R30650_PEA_2_T18 | 2721 | 2876 |
| (SEQ ID NO: 934) | | |

Segment cluster R30650_PEA_{—}2_node_{—}63 (SEQ ID NO:960) according to the present invention is supported by 12 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 43 below describes the starting and ending position of this segment on each transcript.

TABLE 43
Segment location on transcripts

| | | Segment |
|---|---|---|
| | Segment | ending |
| Transcript name | starting position | position |
| R30650_PEA_2_T2 (SEQ ID NO: 929) | 2562 | 2742 |
| R30650_PEA_2_T3 (SEQ ID NO: 930) | 2049 | 2229 |
| R30650_PEA_2_T6 (SEQ ID NO: 931) | 2877 | 3057 |
| R30650_PEA_2_T18 | 2877 | 3057 |
| (SEQ ID NO: 934) | | |

Segment cluster R30650_PEA_{—}2_node_{—}67 (SEQ ID NO:961) according to the present invention is supported by 11 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 44 below describes the starting and ending position of this segment on each transcript.

TABLE 44
Segment location on transcripts

| | | Segment |
|---|---|---|
| | Segment | ending |
| Transcript name | starting position | position |
| R30650_PEA_2_T2 (SEQ ID NO: 929) | 2743 | 2882 |
| R30650_PEA_2_T3 (SEQ ID NO: 930) | 2230 | 2369 |
| R30650_PEA_2_T6 (SEQ ID NO: 931) | 3058 | 3197 |
| R30650_PEA_2_T18 | 3058 | 3197 |
| (SEQ ID NO: 934) | | |

Segment cluster R30650_PEA_{—}2_node_{—}70 (SEQ ID NO:962) according to the present invention is supported by 11 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 45 below describes the starting and ending position of this segment on each transcript.

TABLE 45
Segment location on transcripts

| | | Segment |
|---|---|---|
| | Segment | ending |
| Transcript name | starting position | position |
| R30650_PEA_2_T2 (SEQ ID NO: 929) | 2959 | 3170 |
| R30650_PEA_2_T3 (SEQ ID NO: 930) | 2446 | 2657 |
| R30650_PEA_2_T6 (SEQ ID NO: 931) | 3274 | 3485 |
| R30650_PEA_2_T18 | 3274 | 3485 |
| (SEQ ID NO: 934) | | |

Segment cluster R30650_PEA_{—}2_node_{—}72 (SEQ ID NO:963) according to the present invention is supported by 14 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 46 below describes the starting and ending position of this segment on each transcript.

TABLE 46
Segment location on transcripts

| | | Segment |
|---|---|---|
| | Segment | ending |
| Transcript name | starting position | position |
| R30650_PEA_2_T2 (SEQ ID NO: 929) | 3171 | 3356 |
| R30650_PEA_2_T3 (SEQ ID NO: 930) | 2658 | 2843 |
| R30650_PEA_2_T6 (SEQ ID NO: 931) | 3486 | 3671 |
| R30650_PEA_2_T18 | 3486 | 3671 |
| (SEQ ID NO: 934) | | |

Segment cluster R30650_PEA_{—}2_node_{—}73 (SEQ ID NO:964) according to the present invention is supported by 3 libraries. The number of libraries was determined as previously described. This segment can be found in the following transcript(s): R30650_PEA_{—}2_T18 (SEQ ID NO:934). Table 47 below describes the starting and ending position of this segment on each transcript.

TABLE 47
Segment location on transcripts

| | | Segment |
|---|---|---|
| | Segment | ending |
| Transcript name | starting position | position |
| R30650_PEA_2_T18 | 3672 | 3886 |
| (SEQ ID NO: 934) | | |

Segment cluster R30650_PEA_{—}2_node_{—}75 (SEQ ID NO:965) according to the present invention is supported by 14 libraries. The number of libraries was determined as previously described. This segment can be found in the following transcript(s): R30650_PEA_{—}2_T2 (SEQ ID NO:929) and R30650_PEA_{—}2_T3 (SEQ ID NO:930). Table 48 below describes the starting and ending position of this segment on each transcript.

TABLE 48
Segment location on transcripts

| | | Segment |
|---|---|---|
| | Segment | ending |
| Transcript name | starting position | position |
| R30650_PEA_2_T2 (SEQ ID NO: 929) | 3357 | 3561 |
| R30650_PEA_2_T3 (SEQ ID NO: 930) | 2844 | 3048 |

Segment cluster R30650_PEA_{—}2_node_{—}79 (SEQ ID NO:966) according to the present invention is supported by 15 libraries. The number of libraries was determined as previously described. This segment can be found in the following transcript(s): R30650_PEA_{—}2_T2 (SEQ ID NO:929), R30650_PEA_{—}2_T3 (SEQ ID NO:930) and R30650_PEA_{—}2_T6 (SEQ ID NO:931). Table 49 below describes the starting and ending position of this segment on each transcript.

TABLE 49
Segment location on transcripts

| | | Segment |
|---|---|---|
| | Segment starting | ending |
| Transcript name | position | position |
| R30650_PEA_2_T2 (SEQ ID NO: 929) | 3649 | 3806 |
| R30650_PEA_2_T3 (SEQ ID NO: 930) | 3136 | 3293 |
| R30650_PEA_2_T6 (SEQ ID NO: 931) | 3759 | 3916 |

Segment cluster R30650_PEA_{—}2_node_{—}86 (SEQ ID NO:967) according to the present invention is supported by 43 libraries. The number of libraries was determined as previously described. This segment can be found in the following transcript(s): R30650_PEA_{—}2_T2 (SEQ ID NO:929), R30650_PEA_{—}2_T3 (SEQ ID NO:930) and R30650_PEA_{—}2_T6 (SEQ ID NO:931). Table 50 below describes the starting and ending position of this segment on each transcript.

TABLE 50
Segment location on transcripts

| | | Segment |
|---|---|---|
| | Segment | ending |
| Transcript name | starting position | position |
| R30650_PEA_2_T2 (SEQ ID NO: 929) | 3913 | 4895 |
| R30650_PEA_2_T3 (SEQ ID NO: 930) | 3400 | 4382 |
| R30650_PEA_2_T6 (SEQ ID NO: 931) | 4023 | 5005 |

Segment cluster R30650_PEA_{—}2_node_{—}87 (SEQ ID NO:968) according to the present invention is supported by 43 libraries. The number of libraries was determined as previously described. This segment can be found in the following transcript(s): R30650_PEA_{—}2_T2 (SEQ ID NO:929), R30650_PEA_{—}2_T3 (SEQ ID NO:930) and R30650_PEA_{—}2_T6 (SEQ ID NO:931). Table 51 below describes the starting and ending position of this segment on each transcript.

TABLE 51
Segment location on transcripts

| | | Segment |
|---|---|---|
| | Segment | ending |
| Transcript name | starting position | position |
| R30650_PEA_2_T2 (SEQ ID NO: 929) | 4896 | 5316 |
| R30650_PEA_2_T3 (SEQ ID NO: 930) | 4383 | 4803 |
| R30650_PEA_2_T6 (SEQ ID NO: 931) | 5006 | 5426 |

Segment cluster R30650_PEA_{—}2_node_{—}89 (SEQ ID NO:969) according to the present invention is supported by 69 libraries. The number of libraries was determined as previously described. This segment can be found in the following transcript(s): R30650_PEA_{—}2_T2 (SEQ ID NO:929), R30650_PEA_{—}2_T3 (SEQ ID NO:930) and R30650_PEA_{—}2_T6 (SEQ ID NO:931). Table 52 below describes the starting and ending position of this segment on each transcript.

TABLE 52
Segment location on transcripts

| | | Segment |
|---|---|---|
| | Segment | ending |
| Transcript name | starting position | position |
| R30650_PEA_2_T2 (SEQ ID NO: 929) | 5410 | 5990 |
| R30650_PEA_2_T3 (SEQ ID NO: 930) | 4897 | 5477 |
| R30650_PEA_2_T6 (SEQ ID NO: 931) | 5520 | 6100 |

Segment cluster R30650_PEA_{—}2_node_{—}93 (SEQ ID NO:970) according to the present invention is supported by 108 libraries. The number of libraries was determined as previously described. This segment can be found in the following transcript(s): R30650_PEA_{—}2_T2 (SEQ ID NO:929), R30650_PEA_{—}2_T3 (SEQ ID NO:930) and R30650_PEA_{—}2_T6 (SEQ ID NO:931). Table 53 below describes the starting and ending position of this segment on each transcript.

TABLE 53
Segment location on transcripts

| | | Segment |
|---|---|---|
| | Segment | ending |
| Transcript name | starting position | position |
| R30650_PEA_2_T2 (SEQ ID NO: 929) | 6094 | 6772 |
| R30650_PEA_2_T3 (SEQ ID NO: 930) | 5581 | 6259 |
| R30650_PEA_2_T6 (SEQ ID NO: 931) | 6204 | 6882 |

According to an optional embodiment of the present invention, short segments related to the above cluster are also provided. These segments are up to about 120 bp in length, and so are included in a separate description.

Segment cluster R30650_PEA_{—}2_node_{—}8 (SEQ ID NO:971) according to the present invention is supported by 10 libraries. The number of libraries was determined as previously described. This segment can be found in the following transcript(s): R30650_PEA_{—}2_T14 (SEQ ID NO:932), R30650_PEA_{—}2_T15 (SEQ ID NO:933) and R30650_PEA_{—}2_T21 (SEQ ID NO:935). Table 54 below describes the starting and ending position of this segment on each transcript.

TABLE 54
Segment location on transcripts

| | Segment | Segment |
|---|---|---|
| | starting | ending |
| Transcript name | position | position |
| R30650_PEA_2_T14 (SEQ ID NO: 932) | 1618 | 1712 |
| R30650_PEA_2_T15 (SEQ ID NO: 933) | 1618 | 1712 |
| R30650_PEA_2_T21 (SEQ ID NO: 935) | 1618 | 1712 |

Segment cluster R30650_PEA_{—}2_node_{—}17 (SEQ ID NO:972) according to the present invention is supported by 6 libraries. The number of libraries was determined as previously described. This segment can be found in the following transcript(s): R30650_PEA_{—}2_T6 (SEQ ID NO:931), R30650_PEA_{—}2_T18 (SEQ ID NO:934) and R30650_PEA_{—}2_T23 (SEQ ID NO:936). Table 55 below describes the starting and ending position of this segment on each transcript.

TABLE 55
Segment location on transcripts

| | Segment | Segment |
|---|---|---|
| | starting | ending |
| Transcript name | position | position |
| R30650_PEA_2_T6 (SEQ ID NO: 931) | 249 | 358 |
| R30650_PEA_2_T18 (SEQ ID NO: 934) | 249 | 358 |
| R30650_PEA_2_T23 (SEQ ID NO: 936) | 249 | 358 |

Segment cluster R30650_PEA_{—}2_node_{—}28 (SEQ ID NO:973) according to the present invention is supported by 7 libraries. The number of libraries was determined as previously described. This segment can be found in the following transcript(s): R30650_PEA_{—}2_T6 (SEQ ID NO:931), R30650_PEA_{—}2_T18 (SEQ ID NO:934) and R30650_PEA_{—}2_T23 (SEQ ID NO:936). Table 56 below describes the starting and ending position of this segment on each transcript.

TABLE 56
Segment location on transcripts

| | Segment | Segment |
|---|---|---|
| | starting | ending |
| Transcript name | position | position |
| R30650_PEA_2_T6 (SEQ ID NO: 931) | 1062 | 1132 |
| R30650_PEA_2_T18 (SEQ ID NO: 934) | 1062 | 1132 |
| R30650_PEA_2_T23 (SEQ ID NO: 936) | 1062 | 1132 |

Segment cluster R30650_PEA_{—}2_node_{—}31 (SEQ ID NO:974) according to the present invention is supported by 8 libraries. The number of libraries was determined as previously described. This segment can be found in the following transcript(s): R30650_PEA_{—}2_T6 (SEQ ID NO:931), R30650_PEA_{—}2_T18 (SEQ ID NO:934) and R30650_PEA_{—}2_T23 (SEQ ID NO:936). Table 57 below describes the starting and ending position of this segment on each transcript.

TABLE 57
Segment location on transcripts

| | Segment | Segment |
|---|---|---|
| | starting | ending |
| Transcript name | position | position |
| R30650_PEA_2_T6 (SEQ ID NO: 931) | 1133 | 1228 |
| R30650_PEA_2_T18 (SEQ ID NO: 934) | 1133 | 1228 |
| R30650_PEA_2_T23 (SEQ ID NO: 936) | 1133 | 1228 |

Segment cluster R30650_PEA_{—}2_node_{—}48 (SEQ ID NO:975) according to the present invention is supported by 10 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 58 below describes the starting and ending position of this segment on each transcript.

TABLE 58
Segment location on transcripts

| | Segment | Segment |
|---|---|---|
| | starting | ending |
| Transcript name | position | position |
| R30650_PEA_2_T2 (SEQ ID NO: 929) | 1953 | 2022 |
| R30650_PEA_2_T3 (SEQ ID NO: 930) | 1440 | 1509 |
| R30650_PEA_2_T6 (SEQ ID NO: 931) | 2268 | 2337 |
| R30650_PEA_2_T18 (SEQ ID NO: 934) | 2268 | 2337 |

Segment cluster R30650_PEA_{—}2_node_{—}53 (SEQ ID NO:976) according to the present invention is supported by 12 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 59 below describes the starting and ending position of this segment on each transcript.

TABLE 59
Segment location on transcripts

| | Segment | Segment |
|---|---|---|
| | starting | ending |
| Transcript name | position | position |
| R30650_PEA_2_T2 (SEQ ID NO: 929) | 2152 | 2237 |
| R30650_PEA_2_T3 (SEQ ID NO: 930) | 1639 | 1724 |
| R30650_PEA_2_T6 (SEQ ID NO: 931) | 2467 | 2552 |
| R30650_PEA_2_T18 (SEQ ID NO: 934) | 2467 | 2552 |

Segment cluster R30650_PEA_{—}2_node_{—}58 (SEQ ID NO:977) according to the present invention is supported by 10 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 60 below describes the starting and ending position of this segment on each transcript.

TABLE 60
Segment location on transcripts

| | Segment | Segment |
|---|---|---|
| | starting | ending |
| Transcript name | position | position |
| R30650_PEA_2_T2 (SEQ ID NO: 929) | 2370 | 2405 |
| R30650_PEA_2_T3 (SEQ ID NO: 930) | 1857 | 1892 |
| R30650_PEA_2_T6 (SEQ ID NO: 931) | 2685 | 2720 |
| R30650_PEA_2_T18 (SEQ ID NO: 934) | 2685 | 2720 |

Segment cluster R30650_PEA_{—}2_node_{—}68 (SEQ ID NO:978) according to the present invention is supported by 10 libraries. The number of libraries was determined as previously described. This segment can be found in the following transcript(s): R30650_PEA_{—}2_T2 (SEQ ID NO:929), R30650_PEA_{—}2_T3 (SEQ ID NO:930), R30650_PEA_{—}2_T6 (SEQ ID NO:931) and R30650_PEA_{—}2_T18 (SEQ ID NO:934). Table 61 below describes the starting and ending position of this segment on each transcript.

TABLE 61
Segment location on transcripts

| | Segment | Segment |
|---|---|---|
| | starting | ending |
| Transcript name | position | position |
| R30650_PEA_2_T2 (SEQ ID NO: 929) | 2883 | 2958 |
| R30650_PEA_2_T3 (SEQ ID NO: 930) | 2370 | 2445 |
| R30650_PEA_2_T6 (SEQ ID NO: 931) | 3198 | 3273 |
| R30650_PEA_2_T18 (SEQ ID NO: 934) | 3198 | 3273 |

Segment cluster R30650_PEA_{—}2_node_{—}77 (SEQ ID NO:979) according to the present invention is supported by 13 libraries. The number of libraries was determined as previously described. This segment can be found in the following transcript(s): R30650_PEA_{—}2_T2 (SEQ ID NO:929), R30650_PEA_{—}2_T3 (SEQ ID NO:930) and R30650_PEA_{—}2_T6 (SEQ ID NO:931). Table 62 below describes the starting and ending position of this segment on each transcript.

TABLE 62
Segment location on transcripts

| | | Segment |
|---|---|---|
| | Segment starting | ending |
| Transcript name | position | position |
| R30650_PEA_2_T2 (SEQ ID NO: 929) | 3562 | 3648 |
| R30650_PEA_2_T3 (SEQ ID NO: 930) | 3049 | 3135 |
| R30650_PEA_2_T6 (SEQ ID NO: 931) | 3672 | 3758 |

Segment cluster R30650_PEA_{—}2_node_{—}82 (SEQ ID NO:980) according to the present invention is supported by 20 libraries. The number of libraries was determined as previously described. This segment can be found in the following transcript(s): R30650_PEA_{—}2_T2 (SEQ ID NO:929), R30650_PEA_{—}2_T3 (SEQ ID NO:930) and R30650_PEA_{—}2_T6 (SEQ ID NO:931). Table 63 below describes the starting and ending position of this segment on each transcript.

TABLE 63
Segment location on transcripts

| | | Segment |
|---|---|---|
| | Segment starting | ending |
| Transcript name | position | position |
| R30650_PEA_2_T2 (SEQ ID NO: 929) | 3807 | 3907 |
| R30650_PEA_2_T3 (SEQ ID NO: 930) | 3294 | 3394 |
| R30650_PEA_2_T6 (SEQ ID NO: 931) | 3917 | 4017 |

Segment cluster R30650_PEA_{—}2_node_{—}85 (SEQ ID NO:981) according to the present invention can be found in the following transcript(s): R30650_PEA_{—}2_T2 (SEQ ID NO:929), R30650_PEA_{—}2_T3 (SEQ ID NO:930) and R30650_PEA_{—}2_T6 (SEQ ID NO:931). Table 64 below describes the starting and ending position of this segment on each transcript.

TABLE 64
Segment location on transcripts

| | | Segment |
|---|---|---|
| | Segment | ending |
| Transcript name | starting position | position |
| R30650_PEA_2_T2 (SEQ ID NO: 929) | 3908 | 3912 |
| R30650_PEA_2_T3 (SEQ ID NO: 930) | 3395 | 3399 |
| R30650_PEA_2_T6 (SEQ ID NO: 931) | 4018 | 4022 |

Segment cluster R30650_PEA_{—}2_node_{—}88 (SEQ ID NO:982) according to the present invention is supported by 38 libraries. The number of libraries was determined as previously described. This segment can be found in the following transcript(s): R30650_PEA_{—}2_T2 (SEQ ID NO:929), R30650_PEA_{—}2_T3 (SEQ ID NO:930) and R30650_PEA_{—}2_T6 (SEQ ID NO:931). Table 65 below describes the starting and ending position of this segment on each transcript.

TABLE 65
Segment location on transcripts

| | | Segment |
|---|---|---|
| | Segment | ending |
| Transcript name | starting position | position |
| R30650_PEA_2_T2 (SEQ ID NO: 929) | 5317 | 5409 |
| R30650_PEA_2_T3 (SEQ ID NO: 930) | 4804 | 4896 |
| R30650_PEA_2_T6 (SEQ ID NO: 931) | 5427 | 5519 |

Segment cluster R30650_PEA_{—}2_node_{—}90 (SEQ ID NO:983) according to the present invention can be found in the following transcript(s): R30650_PEA_{—}2_T2 (SEQ ID NO:929), R30650_PEA_{—}2_T3 (SEQ ID NO:930) and R30650_PEA_{—}2_T6 (SEQ ID NO:931). Table 66 below describes the starting and ending position of this segment on each transcript.

TABLE 66
Segment location on transcripts

| | | Segment |
|---|---|---|
| | Segment | ending |
| Transcript name | starting position | position |
| R30650_PEA_2_T2 (SEQ ID NO: 929) | 5991 | 5999 |
| R30650_PEA_2_T3 (SEQ ID NO: 930) | 5478 | 5486 |
| R30650_PEA_2_T6 (SEQ ID NO: 931) | 6101 | 6109 |

Segment cluster R30650_PEA_{—}2_node_{—}91 (SEQ ID NO:984) according to the present invention is supported by 45 libraries. The number of libraries was determined as previously described. This segment can be found in the following transcript(s): R30650_PEA_{—}2_T2 (SEQ ID NO:929), R30650_PEA_{—}2_T3 (SEQ ID NO:930) and R30650_PEA_{—}2_T6 (SEQ ID NO:931). Table 67 below describes the starting and ending position of this segment on each transcript.

TABLE 67
Segment location on transcripts

| | | Segment |
|---|---|---|
| | Segment | ending |
| Transcript name | starting position | position |
| R30650_PEA_2_T2 (SEQ ID NO: 929) | 6000 | 6068 |
| R30650_PEA_2_T3 (SEQ ID NO: 930) | 5487 | 5555 |
| R30650_PEA_2_T6 (SEQ ID NO: 931) | 6110 | 6178 |

Segment cluster R30650_PEA_{—}2_node_{—}92 (SEQ ID NO:985) according to the present invention can be found in the following transcript(s): R30650_PEA_{—}2_T2 (SEQ ID NO:929), R30650_PEA_{—}2_T3 (SEQ ID NO:930) and R30650_PEA_{—}2_T6 (SEQ ID NO:931). Table 68 below describes the starting and ending position of this segment on each transcript.

TABLE 68
Segment location on transcripts

| | | Segment |
|---|---|---|
| | Segment | ending |
| Transcript name | starting position | position |
| R30650_PEA_2_T2 (SEQ ID NO: 929) | 6069 | 6093 |
| R30650_PEA_2_T3 (SEQ ID NO: 930) | 5556 | 5580 |
| R30650_PEA_2_T6 (SEQ ID NO: 931) | 6179 | 6203 |

### Variant Protein Alignment to the Previously Known Protein:

Sequence name: Q9ULM1 (SEQ ID NO:989)
Sequence documentation:
Alignment of: R30650_PEA_2_P4 (SEQ ID NO:991) × Q9ULM1 (SEQ ID NO:989)
Alignment segment 1/1:

| | | | |
|---|---|---|---|
| Quality: | 8887.00 | Escore | 0 |
| Matching length: | 888 | Total length: | 888 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

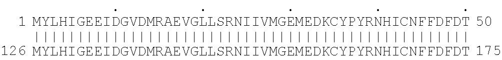

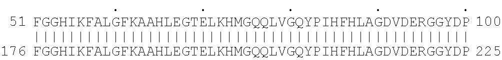

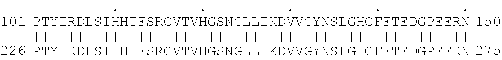

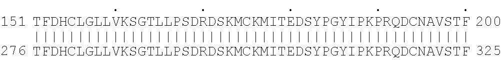

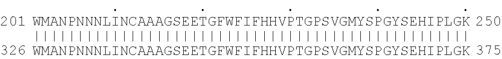

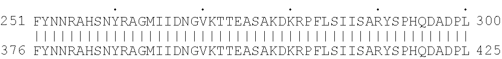

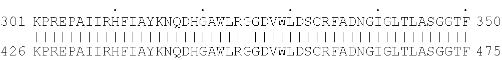

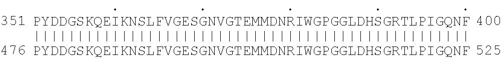

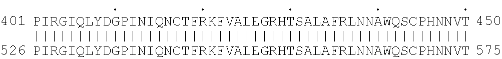

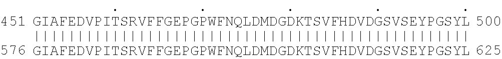

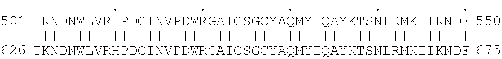

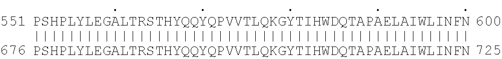

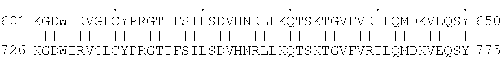

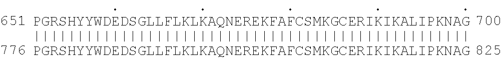

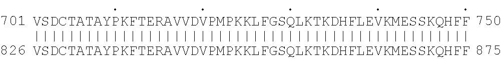

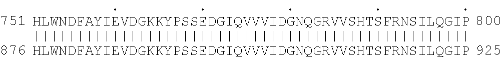

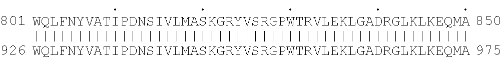

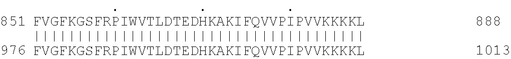

Sequence name: Q8WUJ3 (SEQ ID NO:987)
Sequence documentation:
Alignment of: R30650_PEA_2_P4 (SEQ ID NO:991) × Q8WUJ3 (SEQ ID NO:987) ..
Alignment segment 1/1:

| | | | |
|---|---|---|---|
| Quality: | 5070.00 | Escore | 0 |
| Matching length: | 506 | Total length: | 506 |
| Matching Percent Similarity: | 99.80 | Matching Percent Identity: | 99.80 |
| Total Percent Similarity: | 99.80 | Total Percent Identity: | 99.80 |
| Gaps: | 0 | | |
| Alignment: | | | |

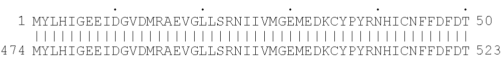

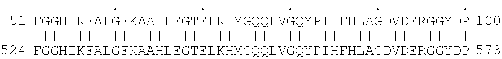

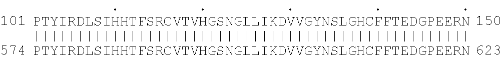

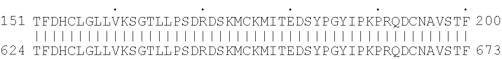

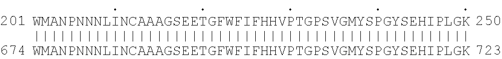

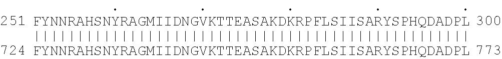

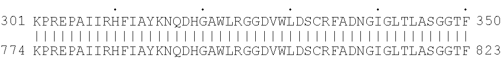

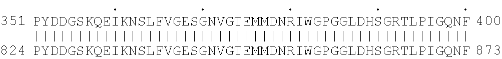

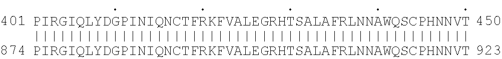

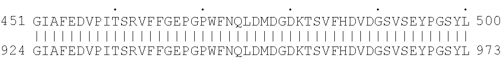

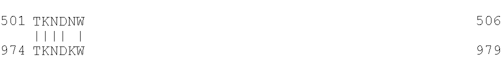

Sequence name: Q9NPN9 (SEQ ID NO:988)
Sequence documentation:
Alignment of: R30650_PEA_2_P4 (SEQ ID NO:991) × Q9NPN9 (SEQ ID NO:988) ..
Alignment segment 1/1:

| | | | |
|---|---|---|---|
| Quality: | 7975.00 | Escore | 0 |
| Matching length: | 797 | Total length: | 797 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

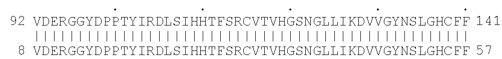

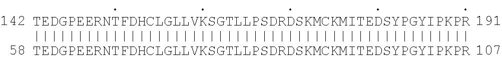

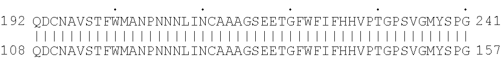

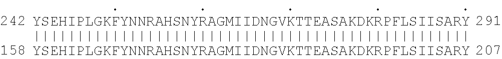

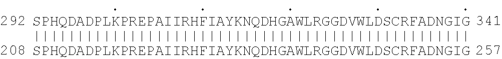

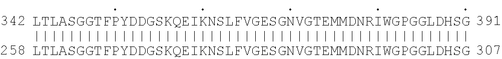

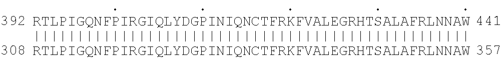

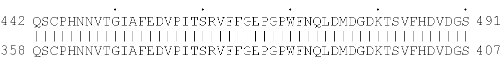

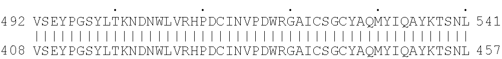

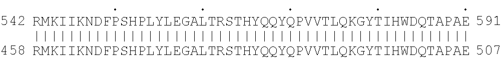

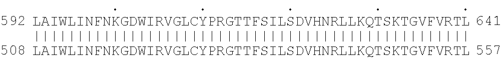

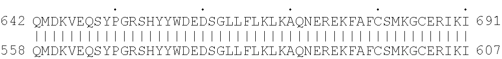

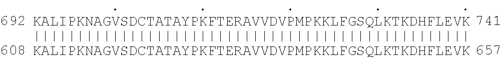

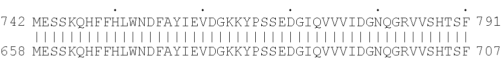

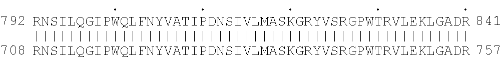

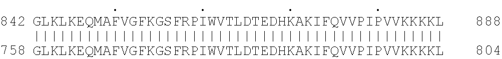

Sequence name: Q9H1K5 (SEQ ID NO:990)
Sequence documentation:
Alignment of: R30650_PEA_2_P4 (SEQ ID NO:991) × Q9HlK5 (SEQ ID NO:990) ..
Alignment segment 1/1:

| | | | |
|---|---|---|---|
| Quality: | 4983.00 | Escore | 0 |
| Matching length: | 499 | Total length: | 499 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

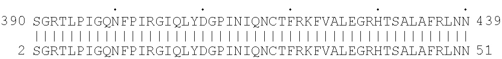

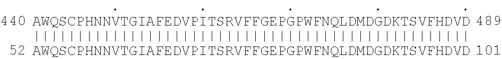

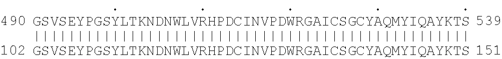

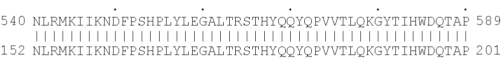

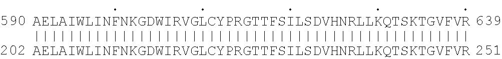

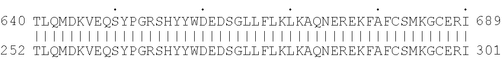

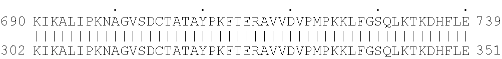

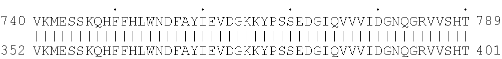

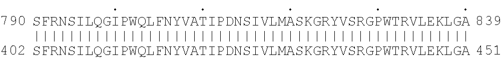

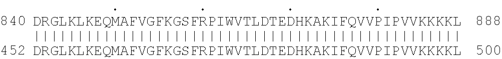

Sequence name: Q9ULM1 (SEQ ID NO:989)
Sequence documentation:
Alignment of: R30650_PEA_2_P5 (SEQ ID NO:992) × Q9ULM1 (SEQ ID NO:989) ..
Alignment segment 1/1:

| | | | |
|---|---|---|---|
| Quality: | 9960.00 | Escore | 0 |
| Matching length: | 996 | Total length: | 996 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

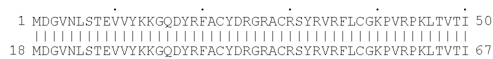

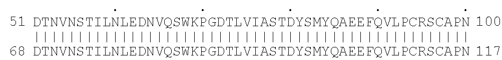

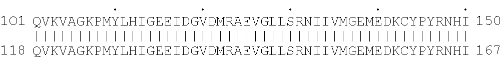

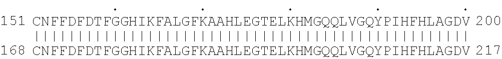

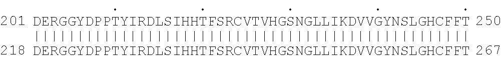

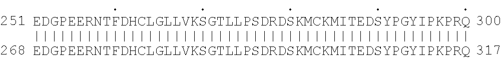

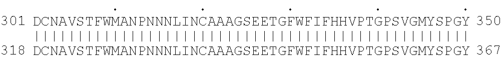

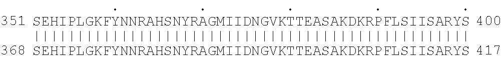

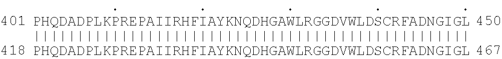

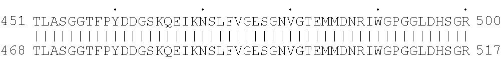

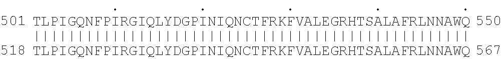

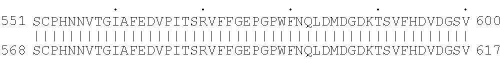

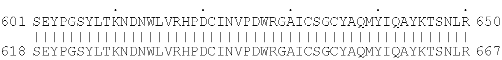

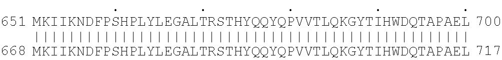

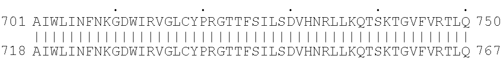

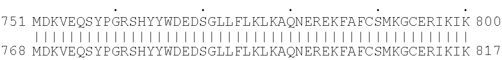

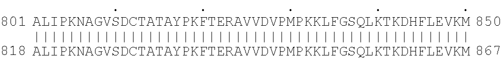

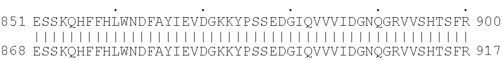

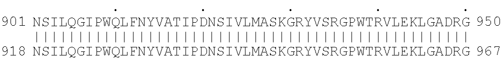

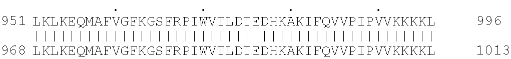

Sequence name: Q8WUJ3 (SEQ ID NO:987)
Sequence documentation:
Alignment of: R30650_PEA_2_P5 (SEQ ID NO:992) × Q8WUJ3 (SEQ ID NO:987) ..
Alignment segment 1/1:

| | | | |
|---|---|---|---|
| Quality: | 6143.00 | Escore | 0 |
| Matching length: | 614 | Total length: | 614 |
| Matching Percent Similarity: | 99.84 | Matching Percent Identity: | 99.84 |
| Total Percent Similarity: | 99.84 | Total Percent Identity: | 99.84 |
| Gaps: | 0 | | |
| Alignment: | | | |

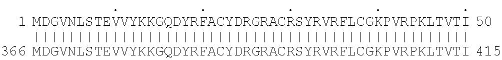

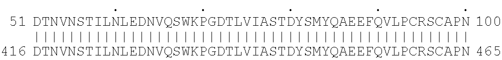

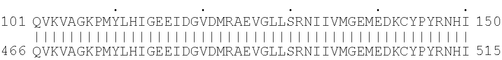

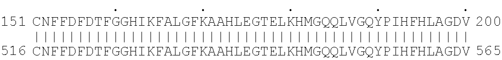

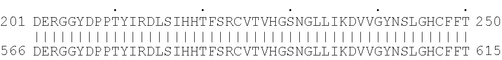

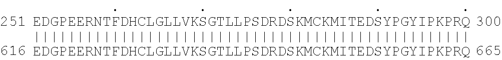

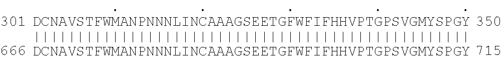

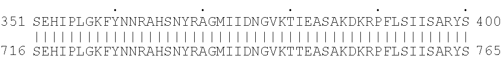

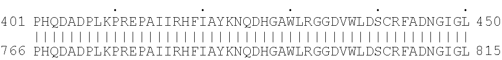

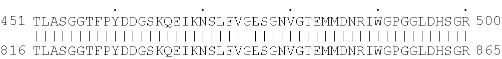

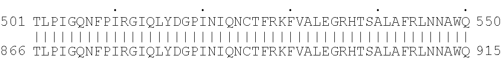

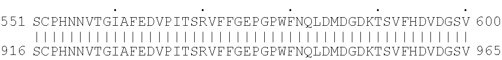

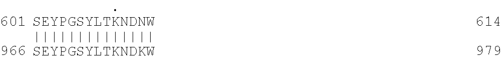

Sequence name: Q9NPN9 (SEQ ID NO:988)
Sequence documentation:
Alignment of: R30650_PEA_2_P5 (SEQ ID NO:992) × Q9NPN9 (SEQ ID NO:988) ..
Alignment segment 1/1:

| | | | |
|---|---|---|---|
| Quality: | 7975.00 | Escore | 0 |
| Matching length: | 797 | Total length: | 797 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

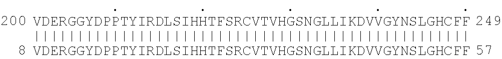

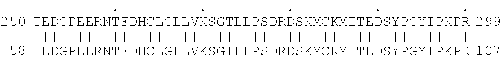

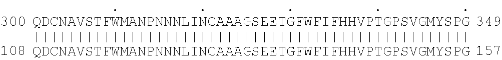

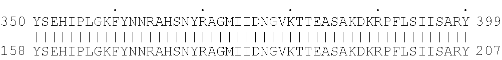

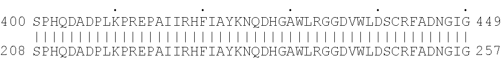

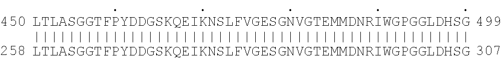

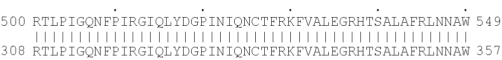

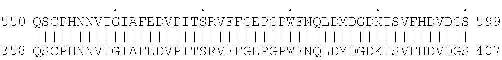

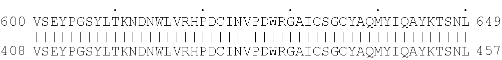

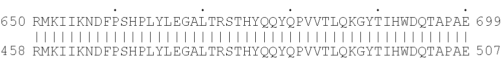

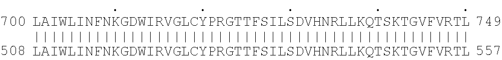

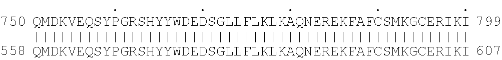

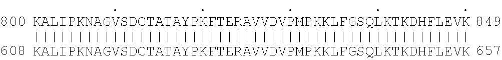

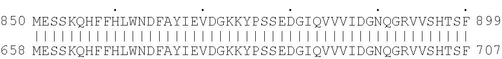

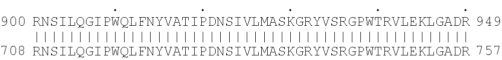

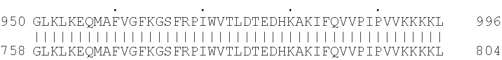

Sequence name: Q9H1K5 (SEQ ID NO:990)
Sequence documentation:
Alignment of: R30650_PEA_2_P5 (SEQ ID NO:992) × Q9HlK5 (SEQ ID NO:990) ..
Alignment segment 1/1:

| | | | |
|---|---|---|---|
| Quality: | 4983.00 | Escore | 0 |
| Matching length: | 499 | Total length: | 499 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

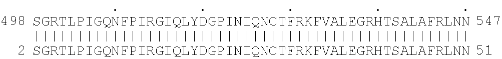

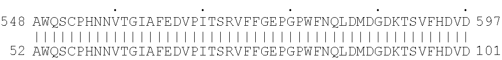

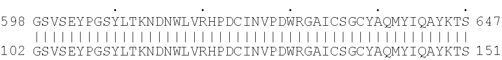

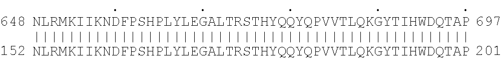

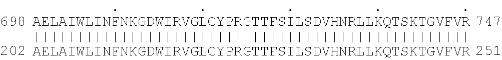

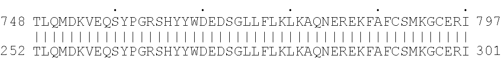

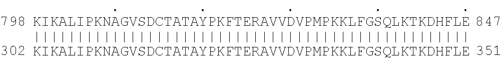

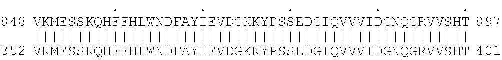

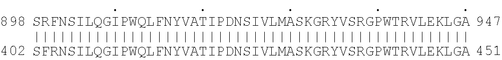

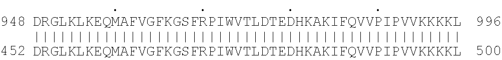

Sequence name: Q9ULM1 (SEQ ID NO:989)
Sequence documentation:
Alignment of: R30650_PEA_2_P8 (SEQ ID NO:993) × Q9ULM1 (SEQ ID NO:989) ..
Alignment segment 1/1:

| | | | |
|---|---|---|---|
| Quality: | 7919.00 | Escore: | 0 |
| Matching length: | 788 | Total length: | 788 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

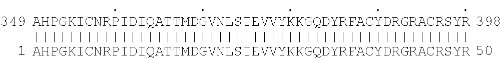

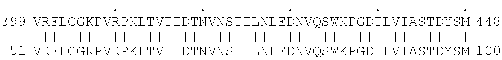

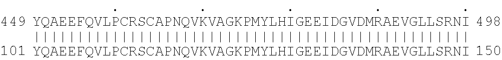

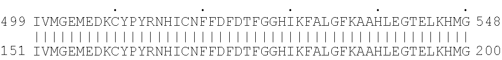

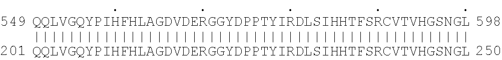

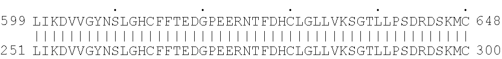

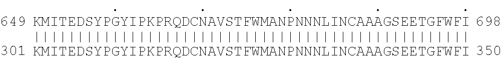

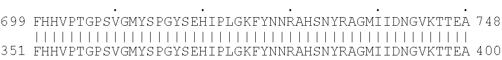

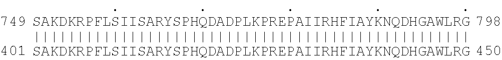

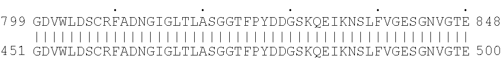

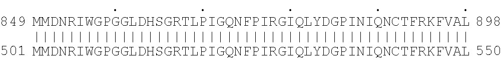

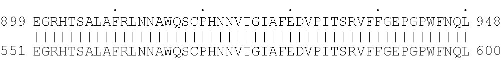

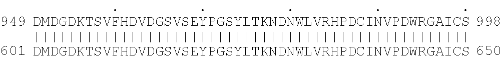

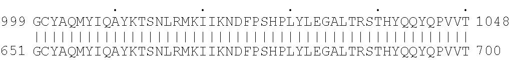

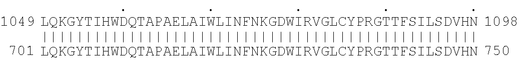

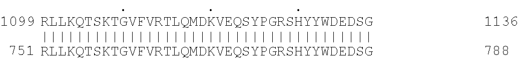

Sequence name: Q8WUJ3 (SEQ ID NO:987)
Sequence documentation:
Alignment of: R30650_PEA_2_P8 (SEQ ID NO:993) × Q8WUJ3 (SEQ ID NO:987) ..
Alignment segment 1/1:

| | | | |
|---|---|---|---|
| Quality: | 9764.00 | Escore | 0 |
| Matching length: | 979 | Total length: | 979 |
| Matching Percent Similarity: | 99.90 | Matching Percent Identity: | 99.90 |
| Total Percent Similarity: | 99.90 | Total Percent Identity: | 99.90 |
| Gaps: | 0 | | |
| Alignment: | | | |

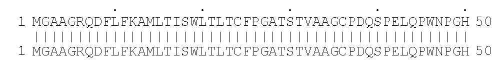

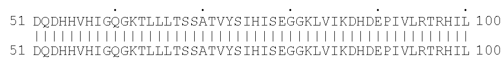

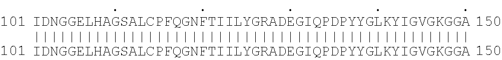

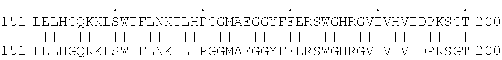

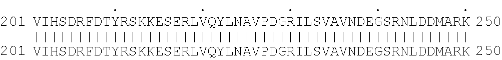

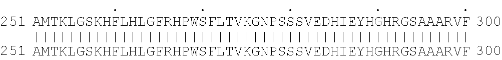

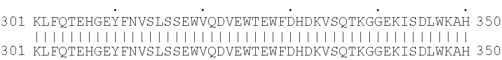

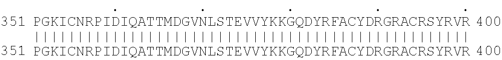

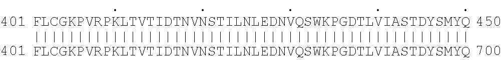

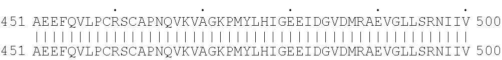

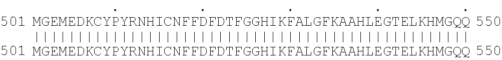

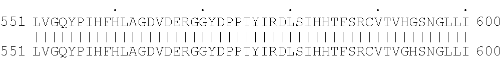

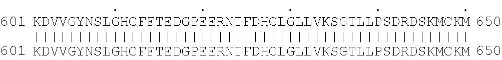

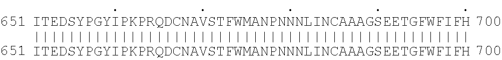

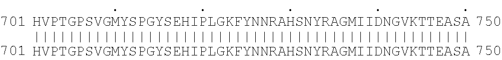

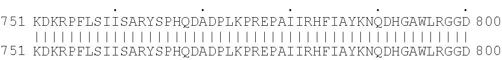

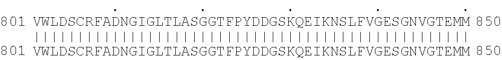

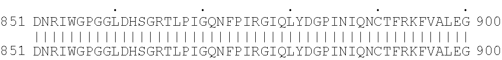

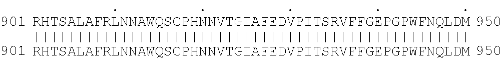

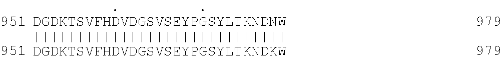

Sequence name: Q9NPN9 (SEQ ID NO:988)
Sequence documentation:
Alignment of: R30650_PEA_2_P8 (SEQ ID NO:993) × Q9NPN9 (SEQ ID NO:988) ..
Alignment segment 1/1:

| | | | |
|---|---|---|---|
| Quality: | 5764.00 | Escore | 0 |
| Matching length: | 572 | Total length: | 572 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

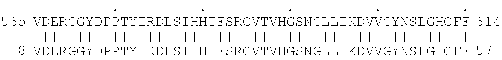

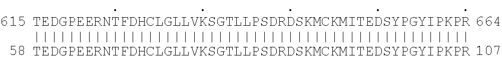

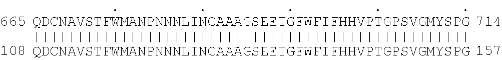

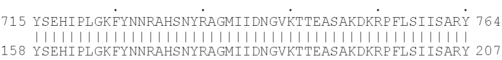

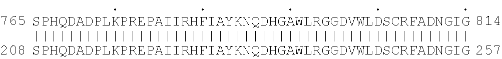

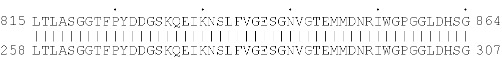

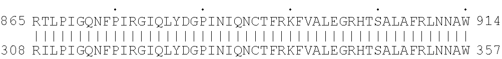

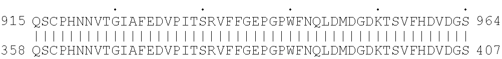

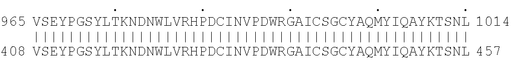

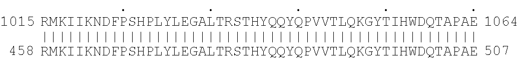

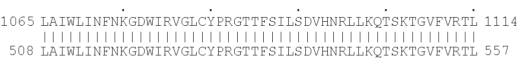

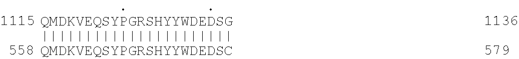

Sequence name: Q9H1K5 (SEQ ID NO:990)
Sequence documentation:
Alignment of: R30650_PEA_2_P15 (SEQ ID NO:993) × Q9H1K5 (SEQ ID NO:990) ..
Alignment segment 1/1:

| | | | |
|---|---|---|---|
| Quality: | 2772.00 | Escore | 0 |
| Matching length: | 274 | Total length: | 274 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

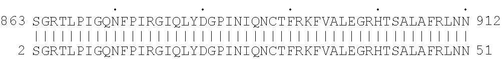

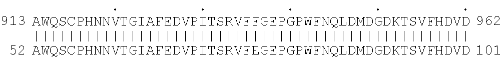

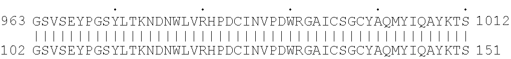

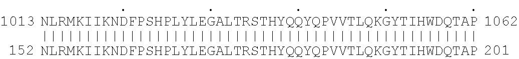

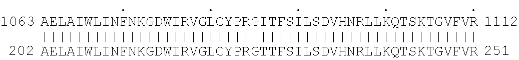

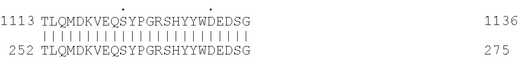

Sequence name: Q9ULM1 (SEQ ID NO:989)
Sequence documentation:
Alignment of: R30650_PEA_2_P15 (SEQ ID NO:996) × Q9ULM1 (SEQ ID NO:989)
Alignment segment 1/1:

| | | | |
|---|---|---|---|
| Quality: | 7919.00 | Escore | 0 |
| Matching length: | 788 | Total length: | 788 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

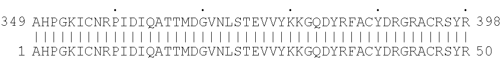

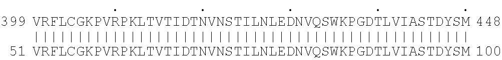

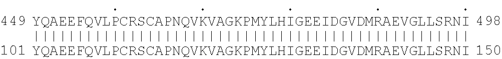

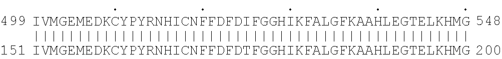

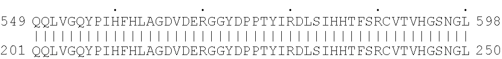

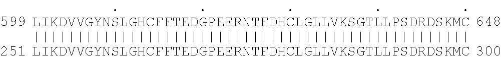

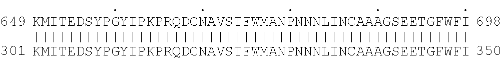

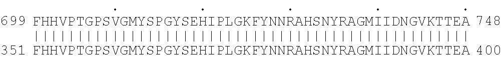

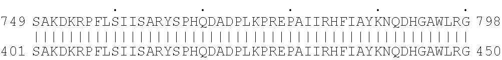

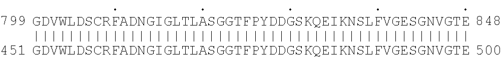

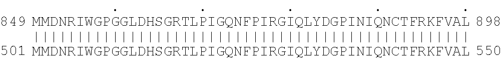

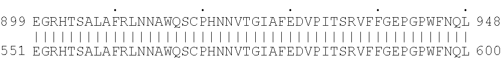

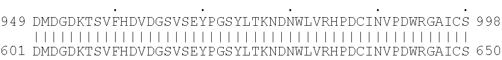

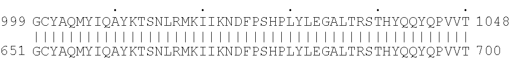

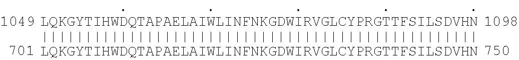

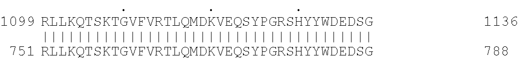

Sequence name: Q8WUJ3 (SEQ ID NO:987)
Sequence documentation:
Alignment of: R30650_PEA_2_P15 (SEQ ID NO:996) × Q8WUJ3 (SEQ ID NO:987)
Alignment segment 1/1:

| | | | |
|---|---|---|---|
| Quality: | 9764.00 | Escore | 0 |
| Matching length: | 979 | Total length: | 979 |
| Matching Percent Similarity: | 99.90 | Matching Percent Identity: | 99.90 |
| Total Percent Similarity: | 99.90 | Total Percent Identity: | 99.90 |
| Gaps: | 0 | | |
| Alignment: | | | |

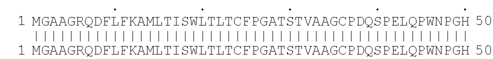

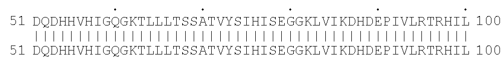

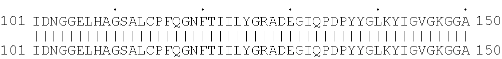

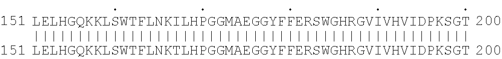

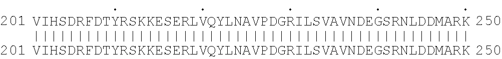

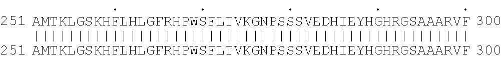

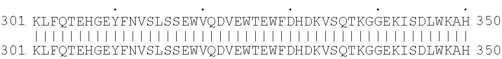

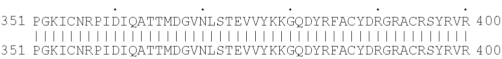

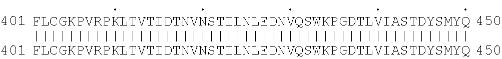

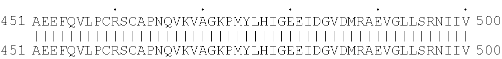

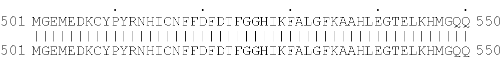

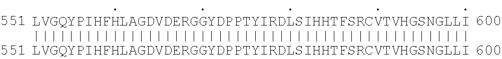

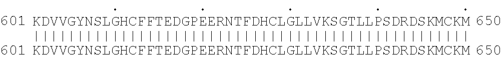

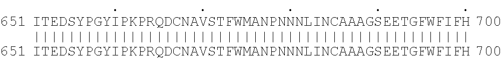

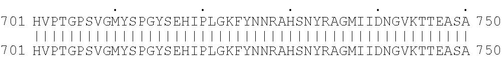

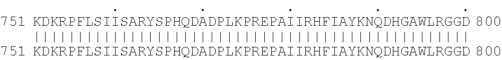

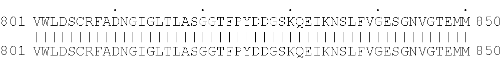

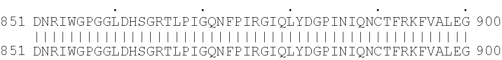

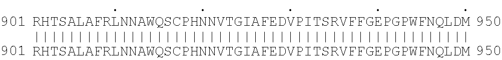

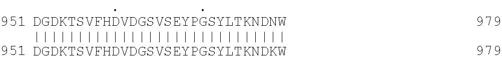

Sequence name: Q9NPN9 (SEQ ID NO:988)
Sequence documentation:
Alignment of: R30650_PEA_2_P15 (SEQ ID NO:996) × Q9NPN9 (SEQ ID NO:988)
Alignment segment 1/1:

| | | | |
|---|---|---|---|
| Quality: | 5764.00 | Escore | 0 |
| Matching length: | 572 | Total length: | 572 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

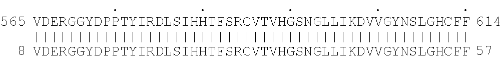

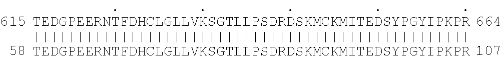

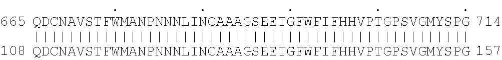

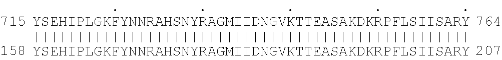

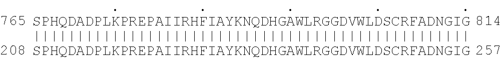

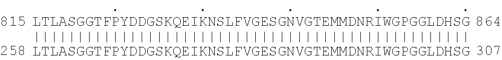

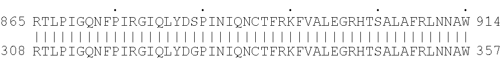

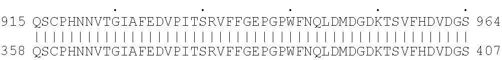

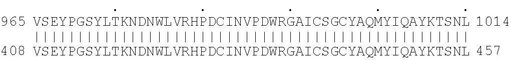

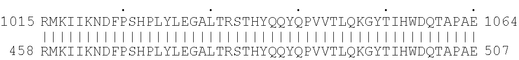

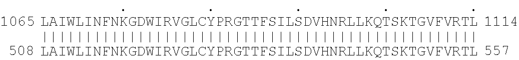

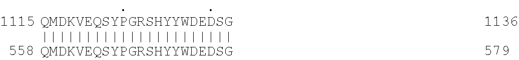

Sequence name: Q9H1K5 (SEQ ID NO:990)
Sequence documentation:
Alignment of: R30650_PEA_2_P15 (SEQ ID NO:996) × Q9H1K5 (SEQ ID NO:990)
Alignment segment 1/1:

| | | | |
|---|---|---|---|
| Quality: | 2772.00 | Escore | 0 |
| Matching length: | 324 | Total length: | 274 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

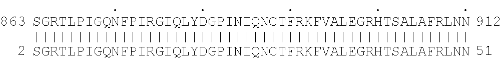

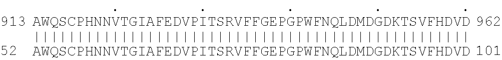

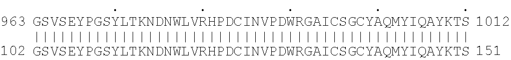

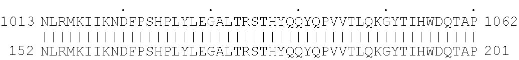

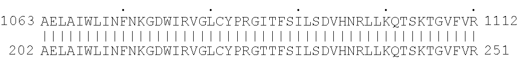

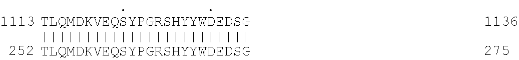

Sequence name: Q8WUJ3 (SEQ ID NO:987)
Sequence documentation:
Alignment of: R30650_PEA_2_P17 (SEQ ID NO:997) × Q8WUJ3 (SEQ ID NO:987)
Alignment segment 1/1:

| | | | |
|---|---|---|---|
| Quality: | 3170.00 | Escore | 0 |
| Matching length: | 324 | Total length: | 324 |
| Matching Percent Similarity: | 99.38 | Matching Percent Identity: | 99.38 |
| Total Percent Similarity: | 99.38 | Total Percent Identity: | 99.38 |
| Gaps: | 0 | | |
| Alignment: | | | |

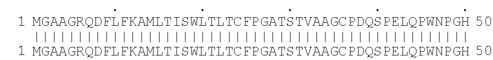

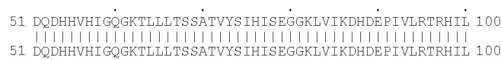

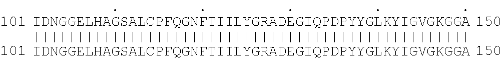

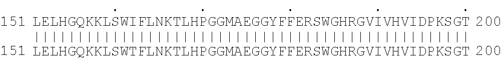

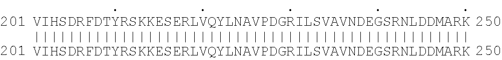

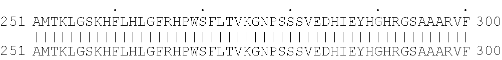

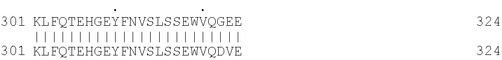

Expression of R30650 Transcripts which are Detectable by Amplicon as Depicted in Sequence Name R30650 Seg76 (SEQ ID NO: 1354) in Normal and Cancerous Colon Tissues

Expression of R30650 transcripts detectable by or according to seg76, R30650 amplicon (SEQ ID NO: 1354) and R30650 F (SEQ ID NO: 1352) and R30650 R (SEQ ID NO: 1353) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

FIG. 57 is a histogram showing over expression of the above-indicated R30650 transcripts in cancerous colon samples relative to the normal samples. The number and percentage of samples that exhibit at least 5 fold over-expression, out of the total number of samples tested is indicated in the bottom.

As is evident from FIG. 57, the expression of R30650 transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 5 fold was found in 18 out of 37 adenocarcinoma samples,

Statistical analysis was applied to verify the significance of these results, as described below.

The P value for the difference in the expression levels of R30650 transcripts detectable by the above amplicon in colon cancer samples versus the normal tissue samples was determined by T test as 1.86E-05.

Threshold of 5 fold overexpression was found to differentiate between cancer and normal samples with P value of 2.42E-03 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: R30650 F forward primer (SEQ ID NO: 1352); and R30650 R reverse primer (SEQ ID NO: 1353).

The present invention also preferably encompasses any amplicon obtained through the use of any suitable primer pair; for example, for the above experiment, the following amplicon was obtained as a non-limiting illustrative example only of a suitable amplicon: R30650 (SEQ ID NO: 1354).

Forward primer (SEQ ID NO: 1352):
CTTCTTGTCCACGGTTTTGTITG
Reverse primer (SEQ ID NO: 1353):
AACATTCCTGGCCACCTGAA
Amplicon (SEQ ID NO: 1354):
CTTCTTGTCCACGGTTTTGTTGAGTTTTCACTCTTCTAATGCAAGGGTCA
CACTGTGAACCACTTAGGATGTGATCACTTTCAGGTGGCCAGGAATGTT

### Description for Cluster T23657

Cluster T23657 features 31 transcript(s) and 33 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| T23657_T0 | 998 |
| T23657_T1 | 999 |
| T23657_T2 | 1000 |
| T23657_T3 | 1001 |
| T23657_T4 | 1002 |
| T23657_T5 | 1003 |
| T23657_T6 | 1004 |
| T23657_T7 | 1005 |
| T23657_T8 | 1006 |
| T23657_T9 | 1007 |
| T23657_T10 | 1008 |
| T23657_T11 | 1009 |
| T23657_T12 | 1010 |
| T23657_T13 | 1011 |
| T23657_T14 | 1012 |
| T23657_T15 | 1013 |
| T23657_T16 | 1014 |
| T23657_T17 | 1015 |
| T23657_T19 | 1016 |
| T23657_T20 | 1017 |
| T23657_T21 | 1018 |
| T23657_T22 | 1019 |
| T23657_T23 | 1020 |
| T23657_T24 | 1021 |
| T23657_T28 | 1022 |
| T23657_T30 | 1023 |
| T23657_T31 | 1024 |
| T23657_T32 | 1025 |
| T23657_T35 | 1026 |
| T23657_T37 | 1027 |
| T23657_T38 | 1028 |

TABLE 2
Segments of interest

| Segment Name | SEQ ID NO: |
|---|---|
| T23657_node_2 | 1029 |
| T23657_node_3 | 1030 |
| T23657_node_8 | 1031 |
| T23657_node_16 | 1032 |
| T23657_node_18 | 1033 |
| T23657_node_23 | 1034 |
| T23657_node_24 | 1035 |
| T23657_node_27 | 1036 |
| T23657_node_29 | 1037 |
| T23657_node_34 | 1038 |
| T23657_node_37 | 1039 |
| T23657_node_38 | 1040 |
| T23657_node_39 | 1041 |
| T23657_node_40 | 1042 |
| T23657_node_45 | 1043 |
| T23657_node_46 | 1044 |
| T23657_node_49 | 1045 |
| T23657_node_0 | 1046 |
| T23657_node_4 | 1047 |
| T23657_node_6 | 1048 |
| T23657_node_11 | 1049 |
| T23657_node_20 | 1050 |
| T23657_node_22 | 1051 |
| T23657_node_25 | 1052 |
| T23657_node_26 | 1053 |
| T23657_node_28 | 1054 |
| T23657_node_30 | 1055 |
| T23657_node_31 | 1056 |
| T23657_node_32 | 1057 |
| T23657_node_41 | 1058 |
| T23657_node_42 | 1059 |
| T23657_node_43 | 1060 |
| T23657_node_44 | 1061 |

TABLE 3
Proteins of interest

| | SEQ | |
|---|---|---|
| Protein Name | ID NO: | Corresponding Transcript(s) |
| T23657_P1 | 1063 | T23657_T0 (SEQ ID NO: 998); |
| | | T23657_T1 (SEQ ID NO: 999); T23657_T8 |
| | | (SEQ ID NO: 1006) |
| T23657_P2 | 1064 | T23657_T2 (SEQ ID NO: 1000); |
| | | T23657_T7 (SEQ ID NO: 1005); |
| | | T23657_T16 |
| | | (SEQ ID NO: 1014); T23657_T20 |
| | | (SEQ ID NO: 1017) |
| T23657_P3 | 1065 | T23657_T3 (SEQ ID NO: 1001); |
| | | T23657_T9 (SEQ ID NO: 1007); |
| | | T23657_T21 |
| | | (SEQ ID NO: 1018) |
| T23657_P4 | 1066 | T23657_T4 (SEQ ID NO: 1002) |
| T23657_P5 | 1067 | T23657_T5 (SEQ ID NO: 1003); |
| | | T23657_T6 (SEQ ID NO: 1004) |
| T23657_P6 | 1068 | T23657_T10 (SEQ ID NO: 1008) |
| T23657_P7 | 1069 | T23657_T12 (SEQ ID NO: 1010); |
| | | T23657_T17 (SEQ ID NO: 1015); |
| | | T23657_T22 (SEQ ID NO: 1019) |
| T23657_P8 | 1070 | T23657_T13 (SEQ ID NO: 1011); |
| | | T23657_T19 (SEQ ID NO: 1016); |
| | | T23657_T28 (SEQ ID NO: 1022) |
| T23657_P9 | 1071 | T23657_T14 (SEQ ID NO: 1012) |
| T23657_P10 | 1072 | T23657_T15 (SEQ ID NO: 1013) |
| T23657_P11 | 1073 | T23657_T23 (SEQ ID NO: 1020) |
| T23657_P12 | 1074 | T23657_T24 (SEQ ID NO: 1021) |
| T23657_P16 | 1075 | T23657_T30 (SEQ ID NO: 1023) |
| T23657_P17 | 1076 | T23657_T31 (SEQ ID NO: 1024); |
| | | T23657_T32 (SEQ ID NO: 1025) |
| T23657_P19 | 1077 | T23657_T35 (SEQ ID NO: 1026) |
| T23657_P21 | 1078 | T23657_T37 (SEQ ID NO: 1027) |
| T23657_P22 | 1079 | T23657_T38 (SEQ ID NO: 1028) |
| T23657_P23 | 1080 | T23657_T11 (SEQ ID NO: 1009) |

These sequences are variants of the known protein Solute carrier family 21 member 12 (SwissProt accession identifier S21C_HUMAN; known also according to the synonyms Sodium-independent organic anion transporter E; Organic anion transporting polypeptide E; OATP-E; Colon organic anion transporter; Organic anion transporter polypeptide-related protein 1; OATP-RP1; OATPRP1; POAT), SEQ ID NO:1062, referred to herein as the previously known protein.

Protein Solute carrier family 21 member 12 (SEQ ID NO:1062) is known or believed to have the following function(s): Mediates the Na(+)-independent transport of organic anions such as the thyroid hormones T3 (triiodo-L-thyronine), T4 (thyroxine) and rT3, and of estrone-3-sulfate and taurocholate. The sequence for protein Solute carrier family 21 member 12 is given at the end of the application, as “Solute carrier family 21 member 12 amino acid sequence”. Known polymorphisms for this sequence are as shown in Table 4.

TABLE 4
Amino acid mutations for Known Protein

| SNP position(s) on amino | |
|---|---|
| acid sequence | Comment |
| 78 | V -> I |
| 246 | T -> M |

Protein Solute carrier family 21 member 12 (SEQ ID NO:1062) localization is believed to be Integral membrane protein.

The following GO Annotation(s) apply to the previously known protein. The following annotation(s) were found: ion transport, which are annotation(s) related to Biological Process; transporter, which are annotation(s) related to Molecular Function; and integral membrane protein, which are annotation(s) related to Cellular Component.

The GO assignment relies on information from one or more of the SwissProt/TremB1 Protein knowledgebase, available from <http://www.expasy.ch/sprot/>; or Locuslink, available from 20<http://www.ncbi.nim.nih.gov/projects/LocusLink/>.

Cluster T23657 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

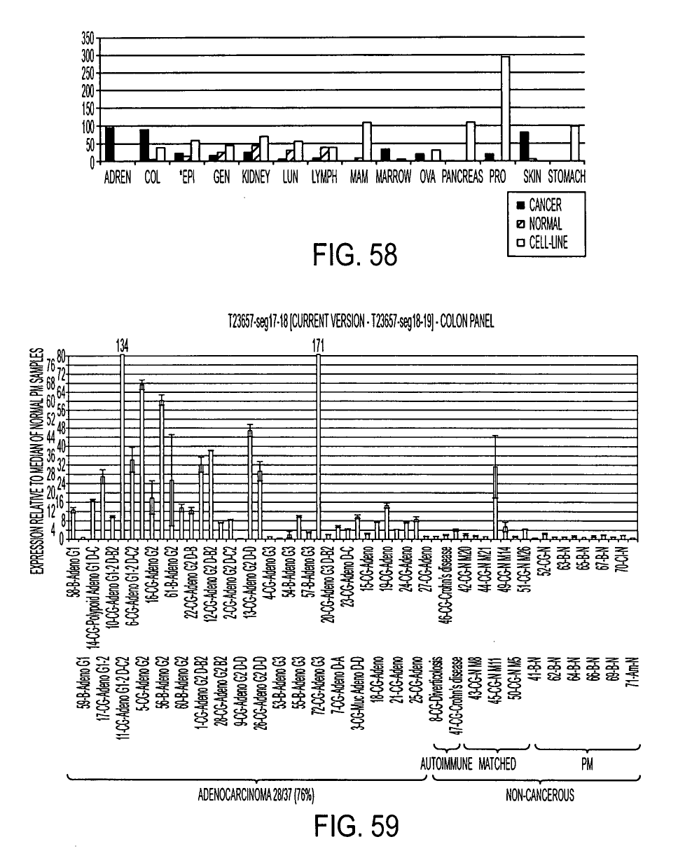

Overall, the following results were obtained as shown with regard to the histograms in FIG. 58 and Table 5. This cluster is overexpressed (at least at a minimum level) in the following pathological conditions: epithelial malignant tumors.

TABLE 5
Normal tissue distribution

| Name of Tissue | Number |
|---|---|
| adrenal | 0 |
| colon | 6 |
| epithelial | 14 |
| general | 26 |
| kidney | 44 |
| lung | 30 |
| lymph nodes | 39 |
| breast | 8 |
| bone marrow | 0 |
| ovary | 0 |
| pancreas | 0 |
| prostate | 0 |
| skin | 5 |
| stomach | 0 |

TABLE 6
P values and ratios for expression in cancerous tissue

| Name of Tissue | P1 | P2 | SP1 | R3 | SP2 | R4 |
|---|---|---|---|---|---|---|
| adrenal | 4.2e−01 | 4.6e−01 | 2.1e−01 | 3.4 | 2.9e−01 | 2.7 |
| colon | 3.1e−02 | 2.9e−02 | 1.2e−01 | 3.2 | 1.6e−01 | 2.8 |
| epithelial | 1.8e−02 | 2.0e−03 | 1.6e−01 | 1.6 | 3.4e−04 | 2.2 |
| general | 2.3e−01 | 3.3e−02 | 9.6e−01 | 0.7 | 2.1e−01 | 1.0 |
| kidney | 7.0e−01 | 5.5e−01 | 9.3e−01 | 0.7 | 7.4e−01 | 1.0 |
| lung | 7.7e−01 | 7.4e−01 | 1 | 0.4 | 6.3e−01 | 0.8 |
| lymph nodes | 6.9e−01 | 7.5e−01 | 1 | 0.5 | 7.9e−01 | 0.8 |
| breast | 9.5e−01 | 5.8e−01 | 1 | 0.8 | 3.1e−01 | 1.7 |
| bone marrow | 4.3e−01 | 4.2e−01 | 1 | 2.1 | 1 | 1.4 |
| ovary | 4.0e−01 | 2.8e−01 | 6.8e−01 | 1.6 | 5.9e−01 | 1.7 |
| pancreas | 1 | 4.4e−01 | 1 | 1.0 | 7.7e−02 | 2.8 |
| prostate | 7.3e−01 | 6.0e−01 | 4.5e−01 | 2.0 | 9.9e−03 | 2.3 |
| skin | 4.0e−01 | 6.8e−01 | 1.4e−01 | 5.0 | 6.4e−01 | 1.1 |
| stomach | 1 | 1.3e−01 | 1 | 1.0 | 1.6e−01 | 2.8 |

As noted above, cluster T23657 features 31 transcript(s), which were listed in Table 1 above. These transcript(s) encode for protein(s) which are variant(s) of protein Solute carrier family 21 member 12 (SEQ ID NO:1062). A description of each variant protein according to the present invention is now provided.

Variant protein T23657_P1 (SEQ ID NO:1063) according to the present invention has an amino acid sequence as given at the end of the application; it is encoded by transcript(s) T23657_T0 (SEQ ID NO:998), T23657_T1 (SEQ ID NO:999) and T23657_T8 (SEQ ID NO:1006). The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein T23657_P1 (SEQ ID NO:1063) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein T23657_P1 (SEQ ID NO:1063) sequence provides support for the deduced sequence of this variant protein according to the present invention).

TABLE 7
Amino acid mutations

| SNP position(s) | | |
|---|---|---|
| on amino acid | Alternative | Previously |
| sequence | amino acid(s) | known SNP? |
| 78 | V -> I | Yes |
| 206 | R -> K | Yes |
| 496 | G -> | No |
| 496 | G -> S | No |

Variant protein T23657_P1 (SEQ ID NO:1063) is encoded by the following transcript(s): T23657_T0 (SEQ ID NO:998), T23657_T1 (SEQ ID NO:999) and T23657_T8 (SEQ ID NO:1006), for which the sequence(s) is/are given at the end of the application.

The coding portion of transcript T23657_T0 (SEQ ID NO:998) is shown in bold; this coding portion starts at position 212 and ends at position 2377. The transcript also has the following SNPs as listed in Table 8 (given according to their position on the nucleotide sequence, with the alternative nucleic acid listed; the last column indicates whether the SNP is known or not; the presence of known SNPs in variant protein T23657_P1 (SEQ ID NO:1063) sequence provides support for the deduced sequence of this variant protein according to the present invention).

TABLE 8
Nucleic acid SNPs

| SNP position | | |
|---|---|---|
| on nucleotide | Alternative | Previously |
| sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2399 | C -> T | No |
| 2402 | C -> | No |
| 2425 | C -> T | Yes |
| 2701 | C -> T | Yes |
| 2714 | C -> T | Yes |

The coding portion of transcript T23657_T1 (SEQ ID NO:999) is shown in bold; this coding portion starts at position 212 and ends at position 2377. The transcript also has the following SNPs as listed in Table 9 (given according to their position on the nucleotide sequence, with the alternative nucleic acid listed; the last column indicates whether the SNP is known or not; the presence of known SNPs in variant protein T23657_P1 (SEQ ID NO:1063) sequence provides support for the deduced sequence of this variant protein according to the present invention).

TABLE 9
Nucleic acid SNPs

| SNP position | | |
|---|---|---|
| on nucleotide | Alternative | Previously |
| sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2399 | C -> T | No |
| 2402 | C -> | No |
| 2425 | C -> T | Yes |
| 2701 | C -> T | Yes |
| 2714 | C -> T | Yes |
| 2877 | C -> T | Yes |
| 2950 | T -> C | Yes |
| 2967 | A -> C | Yes |

The coding portion of transcript T23657_T8 (SEQ ID NO:1006) is shown in bold; this coding portion starts at position 212 and ends at position 2377. The transcript also has the following SNPs as listed in Table 10 (given according to their position on the nucleotide sequence, with the alternative nucleic acid listed; the last column indicates whether the SNP is known or not; the presence of known SNPs in variant protein T23657_P1 (SEQ ID NO:1063) sequence provides support for the deduced sequence of this variant protein according to the present invention).

TABLE 10
Nucleic acid SNPs

| SNP position | | |
|---|---|---|
| on nucleotide | Alternative | Previously |
| sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2399 | C -> T | No |
| 2402 | C -> | No |
| 2425 | C -> T | Yes |

Variant protein T23657_P2 (SEQ ID NO:1064) according to the present invention has an amino acid sequence as given at the end of the application; it is encoded by transcript(s) T23657_T2 (SEQ ID NO:1000), T23657_T7 (SEQ ID NO:1005), T23657_T16 (SEQ ID NO:1014) and T23657_T20 (SEQ ID NO:1017). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P2 (SEQ ID NO:1064) and S21C HUMAN (SEQ ID NO:1062):

1. An isolated chimeric polypeptide encoding for T23657_P2 (SEQ ID NO:1064), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARG THEVRYVSAGQSVACGWWAFAPPCLQVLNTPKGILFFLCAAAFLQGMTVNGFINTVITSLERRYDLHSYQ SGLIASSYDIAACLCLTFVSYFGGSGHKPRWLGWGVLLMGTGSLVFALPHFTAGRYEVELDAGVRTCPAN PGAVCADSTSGLSRYQLVFMLGQFLHGVGATPLYTLGVTYLDENVKSSCSPVYIAIFYTAAILGPAAGYLI GGALLNIYTEMGRRTELTTESPLWVGAWWVGFLGSGAAAFFTAVPILGYPRQLPGSQRYAVMRAAEMH QLKDSSRGEASNPDFGKTIRDLPLSIWLLLKNPTFILLCLAGATEATLITGMSTFSPKFLESQFSLSASEAATL FGYLVVPAGGGGTFLGGFFVNKLRLRGSAVIKFCLFCTVVSLLGILVFSLHCPSVPMAGVTASYGGSLLPE GHLNLTAPCNAACSCQPEHYSPVCGSDGLMYFSLCHAGCPAATETNVDGQKVYRDCSCIPQNLSSGFGHA TAGKCTSTCQRKPLLLVFIFVVIFFTFLSSIPALTATLRCVRDPQRSFALGIQWIVVRILGGIPGPIAFGWVIDK ACLLWQDQCGQQGSCLVYQNSAMSRYILIMGLLYK corresponding to amino acids 1-675 of S21C_HUMAN (SEQ ID NO:1062), which also corresponds to amino acids 1-675 of T23657_P2 (SEQ ID NO:1064), and a second amino acid sequence being at least 70%, optionally at least 80%, preferably at least 85%, more preferably at least 90% and most preferably at least 95% homologous to a polypeptide having the sequence FQLPEVHHSLNVLNRKFQKQTVHNL (SEQ ID NO:1455) corresponding to amino acids 676-700 of T23657_P2 (SEQ ID NO:1064), wherein said first amino acid sequence and second amino acid sequence are contiguous and in a sequential order.

2. An isolated polypeptide encoding for a tail of T23657_P2 (SEQ ID NO:1064), comprising a polypeptide being at least 70%, optionally at least about 80%, preferably at least about 85%, more preferably at least about 90% and most preferably at least about 95% homologous to the sequence FQLPEVHHSLNVLNRKFQKQTVHNL (SEQ ID NO:1455) in T23657_P2 (SEQ ID NO:1064).

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein T23657_P2 (SEQ ID NO:1064) also has the following non-silent SNPs (Single Nucleotide Polymorphisms) as listed in Table 11, (given according to their position(s) on the amino acid sequence, with the alternative amino acid(s) listed; the last column indicates whether the SNP is known or not; the presence of known SNPs in variant protein T23657_P2 (SEQ ID NO:1064) sequence provides support for the deduced sequence of this variant protein according to the present invention).

TABLE 11
Amino acid mutations

| SNP position(s) | | |
|---|---|---|
| on amino acid | Alternative | Previously |
| sequence | amino acid(s) | known SNP? |
| 78 | V -> I | Yes |
| 206 | R -> K | Yes |
| 496 | G -> | No |
| 496 | G -> S | No |

The glycosylation sites of variant protein T23657_P2 (SEQ ID NO:1064), as compared to the known protein Solute carrier family 21 member 12 (SEQ ID NO:1062), are described in Table 12 (given according to their position(s) on the amino acid sequence in the first column; the second column indicates whether the glycosylation site is present in the variant protein; and the last column indicates whether the position is different on the variant protein).

TABLE 12
Glycosylation site(s)

| Position(s) on known | Present in | Position in |
|---|---|---|
| amino acid sequence | variant protein? | variant protein? |
| 557 | yes | 557 |
| 499 | yes | 499 |

Variant protein T23657_P2 (SEQ ID NO:1064) is encoded by the following transcript(s): T23657_T2 (SEQ ID NO:1000), T23657_T7 (SEQ ID NO:1005), T23657_T16 (SEQ ID NO:1014) and T23657_T20 (SEQ ID NO:1017), for which the sequence(s) is/are given at the end of the application.

The coding portion of transcript T23657_T2 (SEQ ID NO:1000) is shown in bold; this coding portion starts at position 212 and ends at position 2311. The transcript also has the following SNPs as listed in Table 13 (given according to their position on the nucleotide sequence, with the alternative nucleic acid listed; the last column indicates whether the SNP is known or not; the presence of known SNPs in variant protein T23657_P2 (SEQ ID NO:1064) sequence provides support for the deduced sequence of this variant protein according to the present invention).

TABLE 13
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2503 | A -> G | Yes |
| 2789 | G -> A | Yes |
| 3444 | C -> T | Yes |
| 3936 | T -> C | Yes |
| 4196 | C -> T | No |
| 4199 | C -> | No |
| 4222 | C -> T | Yes |
| 4498 | C -> T | Yes |
| 4511 | C -> T | Yes |

The coding portion of transcript T23657_T7 (SEQ ID NO:1005) is shown in bold; this coding portion starts at position 212 and ends at position 2311. The transcript also has the following SNPs as listed in Table 14 (given according to their position on the nucleotide sequence, with the alternative nucleic acid listed; the last column indicates whether the SNP is known or not; the presence of known SNPs in variant protein T23657_P2 (SEQ ID NO:1064) sequence provides support for the deduced sequence of this variant protein according to the present invention).

TABLE 14
Nucleic acid SNPs

| SNP position on | Alternative | Previously known |
|---|---|---|
| nucleotide sequence | nucleic acid | SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2503 | A -> G | Yes |
| 2789 | G -> A | Yes |
| 3444 | C -> T | Yes |
| 3936 | T -> C | Yes |
| 4196 | C -> T | No |
| 4199 | C -> | No |
| 4222 | C -> T | Yes |
| 4498 | C -> T | Yes |
| 4511 | C -> T | Yes |
| 4674 | C -> T | Yes |
| 4747 | T -> C | Yes |
| 4764 | A -> C | Yes |

The coding portion of transcript T23657_T16 (SEQ ID NO:1014) is shown in bold; this coding portion starts at position 212 and ends at position 2311. The transcript also has the following SNPs as listed in Table 15 (given according to their position on the nucleotide sequence, with the alternative nucleic acid listed; the last column indicates whether the SNP is known or not; the presence of known SNPs in variant protein T23657_P2 (SEQ ID NO:1064) sequence provides support for the deduced sequence of this variant protein according to the present invention).

TABLE 15
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2503 | A -> G | Yes |
| 2789 | G -> A | Yes |
| 3444 | C -> T | Yes |
| 3936 | T -> C | Yes |
| 4103 | C -> T | No |
| 4106 | C -> | No |
| 4129 | C -> T | Yes |
| 4405 | C -> T | Yes |
| 4418 | C -> T | Yes |

The coding portion of transcript T23657_T20 (SEQ ID NO:1017) is shown in bold; this coding portion starts at position 212 and ends at position 2311. The transcript also has the following SNPs as listed in Table 16 (given according to their position on the nucleotide sequence, with the alternative nucleic acid listed; the last column indicates whether the SNP is known or not; the presence of known SNPs in variant protein T23657_P2 (SEQ ID NO:1064) sequence provides support for the deduced sequence of this variant protein according to the present invention).

TABLE 16
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2569 | C -> T | No |
| 2572 | C -> | No |
| 2595 | C -> T | Yes |
| 2871 | C -> T | Yes |
| 2884 | C -> T | Yes |

Variant protein T23657_P3 (SEQ ID NO:1065) according to the present invention has an amino acid sequence as given at the end of the application; it is encoded by transcript(s) T23657_T3 (SEQ ID NO:1001), T23657_T9 (SEQ ID NO:1007) and T23657_T21 (SEQ ID NO:1018). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P3 (SEQ ID NO:1065) and S21C_HUMAN (SEQ ID NO:1062):

1. An isolated chimeric polypeptide encoding for T23657_P3 (SEQ ID NO:1065), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARG THEVRYVSAGQSVACGWWAFAPPCLQVLNTPKGILFFLCAAAFLQGMTVNGFINTVITSLERRYDLHSYQ SGLIASSYDIAACLCLTFVSYFGGSGHKPRWLGWGVLLMGTGSLVFALPHFTAGRYEVELDAGVRTCPAN PGAVCADSTSGLSRYQLVFMLGQFLHGVGATPLYTLGVTYLDENVKSSCSPVYIAIFYTAAILGPAAGYLI GGALLNIYTEMGRRTELTTESPLWVGAWWVGFLGSGAAAFFTAVPILGYPRQLPGSQRYAVMRAAEMH QLKDSSRGEASNPDFGKTIRDLPLSIWLLLKNPTFILLCLAGATEATLITGMSTFSPKFLESQFSLSASEAATL FGYLVVPAGGGGTFLGGFFVNKLRLRGSAVIKFCLFCTVVSLLGILVFSLHCPSVPMAGVTASYGGSLLPE GHLNLTAPCNAACSCQPEHYSPVCGSDGLMYFSLCHAGCPAATETNVDGQKVYRDCSCIPQNLSSGFGHA TAGKCTSTCQRKPLLLVFIFVVIFFTFLSSIPALTATLRCVRDPQRSFALGIQWIVVRILGGIPGPIAFGWVIDK ACLLWQDQCGQQGSCLVYQNSAMSRYILIMGLLYK corresponding to amino acids 1-675 of S21C_HUMAN (SEQ ID NO:1062), which also corresponds to amino acids 1-675 of T23657_P3 (SEQ ID NO:1065), and a second amino acid sequence being at least 70%, optionally at least 80%, preferably at least 85%, more preferably at least 90% and most preferably at least 95% homologous to a polypeptide having the sequence TIKHKAF corresponding to amino acids 676-682 of T23657_P3 (SEQ ID NO:1065), wherein said first amino acid sequence and second amino acid sequence are contiguous and in a sequential order.

2. An isolated polypeptide encoding for a tail of T23657_P3 (SEQ ID NO:1065), comprising a polypeptide being at least 70%, optionally at least about 80%, preferably at least about 85%, more preferably at least about 90% and most preferably at least about 95% homologous to the sequence TIKHKAF in T23657_P3 (SEQ ID NO:1065).

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein T23657_P3 (SEQ ID NO:1065) also has the following non-silent SNPs (Single Nucleotide Polymorphisms) as listed in Table 17, (given according to their position(s) on the amino acid sequence, with the alternative amino acid(s) listed; the last column indicates whether the SNP is known or not; the presence of known SNPs in variant protein T23657_P3 (SEQ ID NO:1065) sequence provides support for the deduced sequence of this variant protein according to the present invention).

TABLE 17
Amino acid mutations

| SNP position(s) on | Alternative | Previously |
|---|---|---|
| amino acid sequence | amino acid(s) | known SNP? |
| 78 | V -> I | Yes |
| 206 | R -> K | Yes |
| 496 | G -> | No |
| 496 | G -> S | No |

The glycosylation sites of variant protein T23657_P3 (SEQ ID NO:1065), as compared to the known protein Solute carrier family 21 member 12 (SEQ ID NO:1062), are described in Table 18 (given according to their position(s) on the amino acid sequence in the first column; the second column indicates whether the glycosylation site is present in the variant protein; and the last column indicates whether the position is different on the variant protein).

TABLE 18
Glycosylation site(s)

| Position(s) on known | Present in | Position |
|---|---|---|
| amino acid sequence | variant protein? | in variant protein? |
| 557 | yes | 557 |
| 499 | yes | 499 |

Variant protein T23657_P3 (SEQ ID NO:1065) is encoded by the following transcript(s): T23657_T3 (SEQ ID NO:1001), T23657_T9 (SEQ ID NO:1007) and T23657_T21 (SEQ ID NO:1018), for which the sequence(s) is/are given at the end of the application.

The coding portion of transcript T23657_T3 (SEQ ID NO:1001) is shown in bold; this coding portion starts at position 212 and ends at position 2257. The transcript also has the following SNPs as listed in Table 19 (given according to their position on the nucleotide sequence, with the alternative nucleic acid listed; the last column indicates whether the SNP is known or not; the presence of known SNPs in variant protein T23657_P3 (SEQ ID NO:1065) sequence provides support for the deduced sequence of this variant protein according to the present invention).

TABLE 19
Nucleic acid SNPs

| SNP position on | Alternative | Previously known |
|---|---|---|
| nucleotide sequence | nucleic acid | SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2270 | G -> A | Yes |
| 2801 | A -> G | Yes |
| 3087 | G -> A | Yes |
| 3742 | C -> T | Yes |
| 4234 | T -> C | Yes |
| 4494 | C -> T | No |
| 4497 | C -> | No |
| 4520 | C -> T | Yes |
| 4796 | C -> T | Yes |
| 4809 | C -> T | Yes |

The coding portion of transcript T23657_T9 (SEQ ID NO:1007) is shown in bold; this coding portion starts at position 212 and ends at position 2257. The transcript also has the following SNPs as listed in Table 20 (given according to their position on the nucleotide sequence, with the alternative nucleic acid listed; the last column indicates whether the SNP is known or not; the presence of known SNPs in variant protein T23657_P3 (SEQ ID NO:1065) sequence provides support for the deduced sequence of this variant protein according to the present invention).

TABLE 20
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2270 | G -> A | Yes |
| 2801 | A -> G | Yes |
| 3087 | G -> A | Yes |
| 3742 | C -> T | Yes |
| 4234 | T -> C | Yes |
| 4494 | C -> T | No |
| 4497 | C -> | No |
| 4520 | C -> T | Yes |

The coding portion of transcript T23657_T21 (SEQ ID NO:1018) is shown in bold; this coding portion starts at position 212 and ends at position 2257. The transcript also has the following SNPs as listed in Table 21 (given according to their position on the nucleotide sequence, with the alternative nucleic acid listed; the last column indicates whether the SNP is known or not; the presence of known SNPs in variant protein T23657_P3 (SEQ ID NO:1065) sequence provides support for the deduced sequence of this variant protein according to the present invention).

TABLE 21
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2270 | G -> A | Yes |
| 2539 | C -> T | No |
| 2542 | C -> | No |
| 2565 | C -> T | Yes |
| 2841 | C -> T | Yes |
| 2854 | C -> T | Yes |

Variant protein T23657_P4 (SEQ ID NO:1066) according to the present invention has an amino acid sequence as given at the end of the application; it is encoded by transcript(s) T23657_T4 (SEQ ID NO:1002). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P4 (SEQ ID NO:1066) and S21C_HUMAN (SEQ ID NO:1062):

1. An isolated chimeric polypeptide encoding for T23657_P4 (SEQ ID NO:1066), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARG THEVRYVSAGQSVACGWWAFAPPCLQVLNTPKGILFFLCAAAFLQGMTVNGFINTVITSLERRYDLHSYQ SGLIASSYDIAACLCLTFVSYFGGSGHKPRWLGWGVLLMGTGSLVFALPHFTAGRYEVELDAGVRTCPAN PGAVCADSTSGLSRYQLVFMLGQFLHGVGATPLYTLGVTYLDENVKSSCSPVYIAIFYTAAILGPAAGYLI GGALLNIYTEMGRRTELTTESPLWVGAWWVGFLGSGAAAFFTAVPILGYPRQLPGSQRYAVMRAAEMH QLKDSSRGEASNPDFGKTIRDLPLSIWLLLKNPTFILLCLAGATEATLITGMSTFSPKFLESQFSLSASEAATL FGYLVVPAGGGGTFLGGFFVNKLRLRGSAVIKFCLFCTVVSLLGILVFSLHCPSVPMAGVTASYGGSLLPE GHLNLTAPCNAACSCQPEHYSPVCGSDGLMYFSLCHAGCPAATETNVDGQKVYRDCSCIPQNLSSGFGHA TAGKCTSTCQRKPLLLVFIFVVIFFTFLSSIPALTATLRCVRDPQRSFALGIQWIVVRIL corresponding to amino acids 1-625 of S21C_HUMAN (SEQ ID NO:1062), which also corresponds to amino acids 1-625 of T23657_P4 (SEQ ID NO:1066), a second amino acid sequence being at least 70%, optionally at least 80%, preferably at least 85%, more preferably at least 90% and most preferably at least 95% homologous to a polypeptide having the sequence GTVQCEEAMVSCTVCSLHKGM (SEQ ID NO:1574) corresponding to amino acids 626-646 of T23657_P4 (SEQ ID NO:1066), a third amino acid sequence being at least 90% homologous to GGIPGPIAFGWVIDKACLLWQDQCGQQGSCLVYQNSAMSRYILIMGLLYK corresponding to amino acids 626-675 of S21C_HUMAN (SEQ ID NO:1062), which also corresponds to amino acids 647-696 of T23657_P4 (SEQ ID NO:1066), and a fourth amino acid sequence being at least 70%, optionally at least 80%, preferably at least 85%, more preferably at least 90% and most preferably at least 95% homologous to a polypeptide having the sequence TIKHKAF corresponding to amino acids 697-703 of T23657_P4 (SEQ ID NO:1066), wherein said first amino acid sequence, second amino acid sequence, third amino acid sequence and fourth amino acid sequence are contiguous and in a sequential order.

2. An isolated polypeptide encoding for an edge portion of T23657_P4 (SEQ ID NO:1066), comprising an amino acid sequence being at least 70%, optionally at least about 80%, preferably at least about 85%, more preferably at least about 90% and most preferably at least about 95% homologous to the sequence encoding for GTVQCEEAMVSCTVCSLHKGM (SEQ ID NO:1574), corresponding to T23657_P4 (SEQ ID NO:1066).

3. An isolated polypeptide encoding for a tail of T23657_P4 (SEQ ID NO:1066), comprising a polypeptide being at least 70%, optionally at least about 80%, preferably at least about 85%, more preferably at least about 90% and most preferably at least about 95% homologous to the sequence TIKHKAF in T23657_P4 (SEQ ID NO:1066).

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein T23657_P4 (SEQ ID NO:1066) also has the following non-silent SNPs (Single Nucleotide Polymorphisms) as listed in Table 22, (given according to their position(s) on the amino acid sequence, with the alternative amino acid(s) listed; the last column indicates whether the SNP is known or not; the presence of known SNPs in variant protein T23657_P4 (SEQ ID NO:1066) sequence provides support for the deduced sequence of this variant protein according to the present invention).

TABLE 22
Amino acid mutations

| SNP position(s) on | Alternative | Previously |
|---|---|---|
| amino acid sequence | amino acid(s) | known SNP? |
| 78 | V -> I | Yes |
| 206 | R -> K | Yes |
| 496 | G -> | No |
| 496 | G -> S | No |

The glycosylation sites of variant protein T23657_P4 (SEQ ID NO:1066), as compared to the known protein Solute carrier family 21 member 12 (SEQ ID NO:1062), are described in Table 23 (given according to their position(s) on the amino acid sequence in the first column; the second column indicates whether the glycosylation site is present in the variant protein; and the last column indicates whether the position is different on the variant protein).

TABLE 23
Glycosylation site(s)

| Position(s) on known | Present in | Position |
|---|---|---|
| amino acid sequence | variant protein? | in variant protein? |
| 557 | yes | 557 |
| 499 | yes | 499 |

Variant protein T23657_P4 (SEQ ID NO:1066) is encoded by the following transcript(s): T23657_T4 (SEQ ID NO:1002), for which the sequence(s) is/are given at the end of the application. The coding portion of transcript T23657_T4 (SEQ ID NO:1002) is shown in bold; this coding portion starts at position 212 and ends at position 2320. The transcript also has the following SNPs as listed in Table 24 (given according to their position on the nucleotide sequence, with the alternative nucleic acid listed; the last column indicates whether the SNP is known or not; the presence of known SNPs in variant protein T23657_P4 (SEQ ID NO:1066) sequence provides support for the deduced sequence of this variant protein according to the present invention).

TABLE 24
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2333 | G -> A | Yes |
| 2864 | A -> G | Yes |
| 3150 | G -> A | Yes |
| 3805 | C -> T | Yes |
| 4297 | T -> C | Yes |
| 4557 | C -> T | No |
| 4560 | C -> | No |
| 4583 | C -> T | Yes |
| 4859 | C -> T | Yes |
| 4872 | C -> T | Yes |

Variant protein T23657_P5 (SEQ ID NO:1067) according to the present invention has an amino acid sequence as given at the end of the application; it is encoded by transcript(s) T23657_T5 (SEQ ID NO:1003) and T23657_T6 (SEQ ID NO:1004). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P5 (SEQ ID NO:1067) and S21C_HUMAN (SEQ ID NO:1062):

1. An isolated chimeric polypeptide encoding for T23657_P5 (SEQ ID NO:1067), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARG THEVRYVSAGQSVACGWWAFAPPCLQVLNTPKGILFFLCAAAFLQGMTVNGFINTVITSLERRYDLHSYQ SGLIASSYDIAACLCLTFVSYFGGSGHKPRWLGWGVLLMGTGSLVFALPHFTAGRYEVELDAGVRTCPAN PGAVCADSTSGLSRYQLVFMLGQFLHGVGATPLYTLGVTYLDENVKSSCSPVYIAIFYTAAILGPAAGYLI GGALLNIYTEMGRRTELTTESPLWVGAWWVGFLGSGAAAFFTAVPILGYPRQLPGSQRYAVMRAAEMH QLKDSSRGEASNPDFGKTIRDLPLSIWLLLKNPTFILLCLAGATEATLITGMSTFSPKFLESQFSLSASEAATL FGYLVVPAGGGGTFLGGFFVNKLRLRGSAVIKFCLFCTVVSLLGILVFSLHCPSVPMAGVTASYGGSLLPE GHLNLTAPCNAACSCQPEHYSPVCGSDGLMYFSLCHAGCPAATETNVDGQKVYRDCSCIPQNLSSGFGHA TAGKCTSTCQRKPLLLVFIFVVIFFTFLSSIPALTATLR corresponding to amino acids 1-604 of S21C_HUMAN (SEQ ID NO:1062), which also corresponds to amino acids 1-604 of T23657_P5 (SEQ ID NO:1067).

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein T23657_P5 (SEQ ID NO:1067) also has the following non-silent SNPs (Single Nucleotide Polymorphisms) as listed in Table 25, (given according to their position(s) on the amino acid sequence, with the alternative amino acid(s) listed; the last column indicates whether the SNP is known or not; the presence of known SNPs in variant protein T23657_P5 (SEQ ID NO:1067) sequence provides support for the deduced sequence of this variant protein according to the present invention).

TABLE 25
Amino acid mutations

| SNP position(s) on amino | Alternative | Previously |
|---|---|---|
| acid sequence | amino acid(s) | known SNP? |
| 78 | V -> I | Yes |
| 206 | R -> K | Yes |
| 496 | G -> | No |
| 496 | G -> S | No |

The glycosylation sites of variant protein T23657_P5 (SEQ ID NO:1067), as compared to the known protein Solute carrier family 21 member 12 (SEQ ID NO:1062), are described in Table 26 (given according to their position(s) on the amino acid sequence in the first column; the second column indicates whether the glycosylation site is present in the variant protein; and the last column indicates whether the position is different on the variant protein).

TABLE 26
Glycosylation site(s)

| Position(s) on known | Present in | Position |
|---|---|---|
| amino acid sequence | variant protein? | in variant protein? |
| 557 | yes | 557 |
| 499 | yes | 499 |

Variant protein T23657_P5 (SEQ ID NO:1067) is encoded by the following transcript(s): T23657_T5 (SEQ ID NO:1003) and T23657_T6 (SEQ ID NO:1004), for which the sequence(s) is/are given at the end of the application.

The coding portion of transcript T23657_T5 (SEQ ID NO:1003) is shown in bold; this coding portion starts at position 212 and ends at position 2023. The transcript also has the following SNPs as listed in Table 27 (given according to their position on the nucleotide sequence, with the alternative nucleic acid listed; the last column indicates whether the SNP is known or not; the presence of known SNPs in variant protein T23657_P5 (SEQ ID NO:1067) sequence provides support for the deduced sequence of this variant protein according to the present invention).

TABLE 27
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2156 | G -> A | Yes |
| 2795 | A -> G | Yes |
| 3081 | G -> A | Yes |
| 3736 | C -> T | Yes |
| 4228 | T -> C | Yes |
| 4488 | C -> T | No |
| 4491 | C -> | No |
| 4514 | C -> T | Yes |
| 4790 | C -> T | Yes |
| 4803 | C -> T | Yes |

The coding portion of transcript T23657_T6 (SEQ ID NO:1004) is shown in bold; this coding portion starts at position 212 and ends at position 2023. The transcript also has the following SNPs as listed in Table 28 (given according to their position on the nucleotide sequence, with the alternative nucleic acid listed; the last column indicates whether the SNP is known or not; the presence of known SNPs in variant protein T23657_P5 (SEQ ID NO:1067) sequence provides support for the deduced sequence of this variant protein according to the present invention).

TABLE 28
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2156 | G -> A | Yes |
| 2625 | G -> A | Yes |
| 3156 | A -> G | Yes |
| 3442 | G -> A | Yes |
| 4097 | C -> T | Yes |
| 4589 | T -> C | Yes |
| 4849 | C -> T | No |
| 4852 | C -> | No |
| 4875 | C -> T | Yes |
| 5151 | C -> T | Yes |
| 5164 | C -> T | Yes |

Variant protein T23657_P6 (SEQ ID NO:1068) according to the present invention has an amino acid sequence as given at the end of the application; it is encoded by transcript(s) T23657_T10 (SEQ ID NO:1008). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P6 (SEQ ID NO:1068) and S21C_HUMAN (SEQ ID NO:1062):

1. An isolated chimeric polypeptide encoding for T23657_P6 (SEQ ID NO:1068), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARG THEVRYVSAGQSVACGWWAFAPPCLQVLNTPKGILFFLCAAAFLQGMTVNGFINTVITSLERRYDLHSYQ SGLIASSYDIAACLCLTFVSYFGGSGHKPRWLGWGVLLMGTGSLVFALPHFTAGRYEVELDAGVRTCPAN PGAVCADSTSGLSRYQLVFMLGQFLHGVGATPLYTLGVTYLDENVKSSCSPVYIAIFYTAAILGPAAGYLI GGALLNIYTEMGRRTELTTESPLWVGAWWVGFLGSGAAAFFTAVPILGYPRQLPGSQRYAVMRAAEMH QLKDSSRGEASNPDFGKTIRDLPLSIWLLLKNPTFILLCLAGATEATLITGMSTFSPKFLESQFSLSASEAATL FGYLVVPAGGGGTFLGGFFVNKLRLRGSAVIKFCLFCTVVSLLGILVFSLHCPSVPMAGVTASYGGSLLPE GHLNLTAPCNAACSCQPEHYSPVCGSDGLMYFSLCHAGCPAATETNVDGQKV corresponding to amino acids 1-547 of S21C_HUMAN (SEQ ID NO:1062), which also corresponds to amino acids 1-547 of T23657_P6 (SEQ ID NO:1068), and a second amino acid sequence being at least 70%, optionally at least 80%, preferably at least 85%, more preferably at least 90% and most preferably at least 95% homologous to a polypeptide having the sequence SGAAAYRPCPPLDPGKGPPCLPLVIGAIVGLPRCTETVAVSLRIFPLVLAMPLQGNALQLVRESPSFWFSYS L (SEQ ID NO:1458) corresponding to amino acids 548-620 of T23657_P6 (SEQ ID NO:1068), wherein said first amino acid sequence and second amino acid sequence are contiguous and in a sequential order.

2. An isolated polypeptide encoding for a tail of T23657_P6 (SEQ ID NO:1068), comprising a polypeptide being at least 70%, optionally at least about 80%, preferably at least about 85%, more preferably at least about 90% and most preferably at least about 95% homologous to the sequence SGAAAYRPCPPLDPGKGPPCLPLVIGAIVGLPRCTETVAVSLRIFPLVLAMPLQGNALQLVRESPSFWFSYS L (SEQ ID NO:1458) in T23657_P6 (SEQ ID NO:1068).

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein T23657_P6 (SEQ ID NO:1068) also has the following non-silent SNPs (Single Nucleotide Polymorphisms) as listed in Table 29, (given according to their position(s) on the amino acid sequence, with the alternative amino acid(s) listed; the last column indicates whether the SNP is known or not; the presence of known SNPs in variant protein T23657_P6 (SEQ ID NO:1068) sequence provides support for the deduced sequence of this variant protein according to the present invention).

TABLE 29
Amino acid mutations

| SNP position on | Alternative | Previously |
|---|---|---|
| amino acid sequence | amino acid | known SNP? |
| 78 | V -> I | Yes |
| 206 | R -> K | Yes |
| 496 | G -> | No |
| 496 | G -> S | No |
| 573 | G -> R | Yes |

The glycosylation sites of variant protein T23657_P6 (SEQ ID NO:1068), as compared to the known protein Solute carrier family 21 member 12 (SEQ ID NO:1062), are described in Table 30 (given according to their position(s) on the amino acid sequence in the first column; the second column indicates whether the glycosylation site is present in the variant protein; and the last column indicates whether the position is different on the variant protein).

TABLE 30
Glycosylation site(s)

| Position(s) on known | Present in | Position in |
|---|---|---|
| amino acid sequence | variant protein? | variant protein? |
| 557 | no | |
| 499 | yes | 499 |

Variant protein T23657_P6 (SEQ ID NO:1068) is encoded by the following transcript(s): T23657_T10 (SEQ ID NO:1008), for which the sequence(s) is/are given at the end of the application. The coding portion of transcript T23657_T10 (SEQ ID NO:1008) is shown in bold; this coding portion starts at position 212 and ends at position 2071. The transcript also has the following SNPs as listed in Table 31 (given according to their position on the nucleotide sequence, with the alternative nucleic acid listed; the last column indicates whether the SNP is known or not; the presence of known SNPs in variant protein T23657_P6 (SEQ ID NO:1068) sequence provides support for the deduced sequence of this variant protein according to the present invention).

TABLE 31
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 1928 | G -> A | Yes |
| 2257 | G -> A | Yes |
| 2896 | A -> G | Yes |
| 3182 | G -> A | Yes |
| 3837 | C -> T | Yes |
| 4329 | T -> C | Yes |
| 4589 | C -> T | No |
| 4592 | C -> | No |
| 4615 | C -> T | Yes |
| 4891 | C -> T | Yes |
| 4904 | C -> T | Yes |

Variant protein T23657_P7 (SEQ ID NO:1069) according to the present invention has an amino acid sequence as given at the end of the application; it is encoded by transcript(s) T23657_T12 (SEQ ID NO:1010), T23657_T17 (SEQ ID NO:1015) and T23657_T22 (SEQ ID NO:1019). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P7 (SEQ ID NO:1069) and S21C_HUMAN (SEQ ID NO:1062):

1. An isolated chimeric polypeptide encoding for T23657_P7 (SEQ ID NO:1069), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARG THEVRYVSAGQSVACGWWAFAPPCLQVLNTPKGILFFLCAAAFLQGMTVNGFINTVITSLERRYDLHSYQ SGLIASSYDIAACLCLTFVSYFGGSGHKPRWLGWGVLLMGTGSLVFALPHFTAGRYEVELDAGVRTCPAN PGAVCADSTSGLSRYQLVFMLGQFLHGVGATPLYTLGVTYLDENVKSSCSPVYIAIFYTAAILGPAAGYLI GGALLNIYTEMGRRTELTTESPLWVGAWWVGFLGSGAAAFFTAVPILGYPRQLPGSQRYAVMRAAEMH QLKDSSRGEASNPDFGKTIRDLPLSIWLLLKNPTFILLCLAGATEATLITGMSTFSPKFLESQFSLSASEAATL FGYLVVPAGGGGTFLGGFFVNKLRLRGSAVIKFCLFCTVVSLLGILVFSLHCPSVPMAGVTASYGGSLLPE GHLNLTAPCNAACSCQPEHYSPVCGSDGLMYFSLCHAGCPAATETNVDGQK corresponding to amino acids 1-546 of S21C_HUMAN (SEQ ID NO:1062), which also corresponds to amino acids 1-546 of T23657_P7 (SEQ ID NO:1069), and a second amino acid sequence being at least 70%, optionally at least 80%, preferably at least 85%, more preferably at least 90% and most preferably at least 95% homologous to a polypeptide having the sequence MCP corresponding to amino acids 547-549 of T23657_P7 (SEQ ID NO:1069), wherein said first amino acid sequence and second amino acid sequence are contiguous and in a sequential order.

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein T23657_P7 (SEQ ID NO:1069) also has the following non-silent SNPs (Single Nucleotide Polymorphisms) as listed in Table 32, (given according to their position(s) on the amino acid sequence, with the alternative amino acid(s) listed; the last column indicates whether the SNP is known or not; the presence of known SNPs in variant protein T23657_P7 (SEQ ID NO:1069) sequence provides support for the deduced sequence of this variant protein according to the present invention).

TABLE 32
Amino acid mutations

| SNP position on | Alternative | Previously |
|---|---|---|
| amino sequence | amino acid | known SNP? |
| 78 | V -> I | Yes |
| 206 | R -> K | Yes |
| 496 | G -> | No |
| 496 | G -> S | No |

The glycosylation sites of variant protein T23657_P7 (SEQ ID NO:1069), as compared to the known protein Solute carrier family 21 member 12 (SEQ ID NO:1062), are described in Table 33 (given according to their position(s) on the amino acid sequence in the first column; the second column indicates whether the glycosylation site is present in the variant protein; and the last column indicates whether the position is different on the variant protein).

TABLE 33
Glycosylation site(s)

| Position(s) on known | Present in | Position in |
|---|---|---|
| amino acid sequence | variant protein? | variant protein? |
| 557 | no | |
| 499 | yes | 499 |

Variant protein T23657_P7 (SEQ ID NO:1069) is encoded by the following transcript(s): T23657_T12 (SEQ ID NO:1010), T23657_T17 (SEQ ID NO:1015) and T23657_T22 (SEQ ID NO:1019), for which the sequence(s) is/are given at the end of the application.

The coding portion of transcript T23657_T12 (SEQ ID NO:1010) is shown in bold; this coding portion starts at position 212 and ends at position 1858. The transcript also has the following SNPs as listed in Table 34 (given according to their position on the nucleotide sequence, with the alternative nucleic acid listed; the last column indicates whether the SNP is known or not; the presence of known SNPs in variant protein T23657_P7 (SEQ ID NO:1069) sequence provides support for the deduced sequence of this variant protein according to the present invention).

TABLE 34
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2330 | A -> G | Yes |
| 2616 | G -> A | Yes |
| 3271 | C -> T | Yes |
| 3763 | T -> C | Yes |
| 4023 | C -> T | No |
| 4026 | C -> | No |
| 4049 | C -> T | Yes |
| 4325 | C -> T | Yes |
| 4338 | C -> T | Yes |

The coding portion of transcript T23657_T17 (SEQ ID NO:1015) is shown in bold; this coding portion starts at position 212 and ends at position 1858. The transcript also has the following SNPs as listed in Table 35 (given according to their position on the nucleotide sequence, with the alternative nucleic acid listed; the last column indicates whether the SNP is known or not; the presence of known SNPs in variant protein T23657_P7 (SEQ ID NO:1069) sequence provides support for the deduced sequence of this variant protein according to the present invention).

TABLE 35
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2226 | C -> T | No |
| 2229 | C -> | No |
| 2252 | C -> T | Yes |
| 2528 | C -> T | Yes |
| 2541 | C -> T | Yes |

The coding portion of transcript T23657_T22 (SEQ ID NO:1019) is shown in bold; this coding portion starts at position 212 and ends at position 1858. The transcript also has the following SNPs as listed in Table 36 (given according to their position on the nucleotide sequence, with the alternative nucleic acid listed; the last column indicates whether the SNP is known or not; the presence of known SNPs in variant protein T23657_P7 (SEQ ID NO:1069) sequence provides support for the deduced sequence of this variant protein according to the present invention).

TABLE 36
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2396 | C -> T | No |
| 2399 | C -> | No |
| 2422 | C -> T | Yes |
| 2698 | C -> T | Yes |
| 2711 | C -> T | Yes |

Variant protein T23657_P8 (SEQ ID NO:1070) according to the present invention has an amino acid sequence as given at the end of the application; it is encoded by transcript(s) T23657_T13 (SEQ ID NO:1011), T23657_T19 (SEQ ID NO:1016) and T23657_T28 (SEQ ID NO:1022). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P8 (SEQ ID NO:1070) and S21C_HUMAN (SEQ ID NO:1062):

1. An isolated chimeric polypeptide encoding for T23657_P8 (SEQ ID NO:1070), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARG THEVRYVSAGQSVACGWWAFAPPCLQVLNTPKGILFFLCAAAFLQGMTVNGFINTVITSLERRYDLHSYQ SGLIASSYDIAACLCLTFVSYFGGSGHKPRWLGWGVLLMGTGSLVFALPHFTAGRYEVELDAGVRTCPAN PGAVCADSTSGLSRYQLVFMLGQFLHGVGATPLYTLGVTYLDENVKSSCSPVYIAIFYTAAILGPAAGYLI GGALLNIYTEMGRRTELTTESPLWVGAWWVGFLGSGAAAFFTAVPILGYPRQLPGSQRYAVMRAAEMH QLKDSSRGEASNPDFGKTIRDLPLSIWLLLKNPTFILLCLAGATEATLITGMSTFSPKFLESQFSLSASEAATL FGYLVVPAGGGGTFLGGFFVNKLRLRGSAVIKFCLFCTVVSLLGILVFSLHCPSVPMAGVTASYGGSLLPE GHLNLTAPCNAACSCQPEHYSPVCGSDGLMYFSLCHAGCPAATETNVDGQK corresponding to amino acids 1-546 of S21C_HUMAN (SEQ ID NO:1062), which also corresponds to amino acids 1-546 of T23657_P8 (SEQ ID NO:1070), and a second amino acid sequence being at least 70%, optionally at least 80%, preferably at least 85%, more preferably at least 90% and most preferably at least 95% homologous to a polypeptide having the sequence QHSCTNGNSTMCP (SEQ ID NO:1459) corresponding to amino acids 547-559 of T23657_P8 (SEQ ID NO:1070), wherein said first amino acid sequence and second amino acid sequence are contiguous and in a sequential order.

2. An isolated polypeptide encoding for a tail of T23657_P8 (SEQ ID NO:1070), comprising a polypeptide being at least 70%, optionally at least about 80%, preferably at least about 85%, more preferably at least about 90% and most preferably at least about 95% homologous to the sequence QHSCTNGNSTMCP (SEQ ID NO:1459) in T23657_P8 (SEQ ID NO:1070).

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein T23657_P8 (SEQ ID NO:1070) also has the following non-silent SNPs (Single Nucleotide Polymorphisms) as listed in Table 37, (given according to their position(s) on the amino acid sequence, with the alternative amino acid(s) listed; the last column indicates whether the SNP is known or not; the presence of known SNPs in variant protein T23657_P8 (SEQ ID NO:1070) sequence provides support for the deduced sequence of this variant protein according to the present invention).

TABLE 37
Amino acid mutations

| SNP position(s) on | Alternative | Previously |
|---|---|---|
| amino acid sequence | amino acid(s) | known SNP? |
| 78 | V -> I | Yes |
| 206 | R -> K | Yes |
| 496 | G -> | No |
| 496 | G -> S | No |

The glycosylation sites of variant protein T23657_P8 (SEQ ID NO:1070), as compared to the known protein Solute carrier family 21 member 12 (SEQ ID NO:1062), are described in Table 38 (given according to their position(s) on the amino acid sequence in the first column; the second column indicates whether the glycosylation site is present in the variant protein; and the last column indicates whether the position is different on the variant protein).

TABLE 38
Glycosylation site(s)

| Position(s) on known | Present in | Position in |
|---|---|---|
| amino acid sequence | variant protein? | variant protein? |
| 557 | no | |
| 499 | yes | 499 |

Variant protein T23657_P8 (SEQ ID NO:1070) is encoded by the following transcript(s): T23657_T13 (SEQ ID NO:1011), T23657_T19 (SEQ ID NO:1016) and T23657_T28 (SEQ ID NO:1022), for which the sequence(s) is/are given at the end of the application.

The coding portion of transcript T23657_T13 (SEQ ID NO:1011) is shown in bold; this coding portion starts at position 212 and ends at position 1888. The transcript also has the following SNPs as listed in Table 39 (given according to their position on the nucleotide sequence, with the alternative nucleic acid listed; the last column indicates whether the SNP is known or not; the presence of known SNPs in variant protein T23657_P8 (SEQ ID NO:1070) sequence provides support for the deduced sequence of this variant protein according to the present invention).

TABLE 39
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2127 | G -> A | Yes |
| 2658 | A -> G | Yes |
| 2944 | G -> A | Yes |
| 3599 | C -> T | Yes |
| 4091 | T -> C | Yes |
| 4351 | C -> T | No |
| 4354 | C -> | No |
| 4377 | C -> T | Yes |
| 4653 | C -> T | Yes |
| 4666 | C -> T | Yes |

The coding portion of transcript T23657_T19 (SEQ ID NO:1016) is shown in bold; this coding portion starts at position 212 and ends at position 1888. The transcript also has the following SNPs as listed in Table 40 (given according to their position on the nucleotide sequence, with the alternative nucleic acid listed; the last column indicates whether the SNP is known or not; the presence of known SNPs in variant protein T23657_P8 (SEQ ID NO:1070) sequence provides support for the deduced sequence of this variant protein according to the present invention).

TABLE 40
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2256 | C -> T | No |
| 2259 | C -> | No |
| 2282 | C -> T | Yes |
| 2558 | C -> T | Yes |
| 2571 | C -> T | Yes |

The coding portion of transcript T23657_T28 (SEQ ID NO:1022) is shown in bold; this coding portion starts at position 212 and ends at position 1888. The transcript also has the following SNPs as listed in Table 41 (given according to their position on the nucleotide sequence, with the alternative nucleic acid listed; the last column indicates whether the SNP is known or not; the presence of known SNPs in variant protein T23657_P8 (SEQ ID NO:1070) sequence provides support for the deduced sequence of this variant protein according to the present invention).

TABLE 41
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2308 | C -> T | Yes |
| 2442 | G -> A | Yes |

Variant protein T23657_P9 (SEQ ID NO:1071) according to the present invention has an amino acid sequence as given at the end of the application; it is encoded by transcript(s) T23657_T14 (SEQ ID NO:1012). The location of the variant protein was determined according to results from a number of different software programs and analyses, including analyses from SignalP and other specialized programs. The variant protein is believed to be located as follows with regard to the cell: secreted. The protein localization is believed to be secreted because one of the two signal-peptide prediction programs (HMM:Signal peptide,NN:NO) predicts that this protein has a signal peptide.

Variant protein T23657_P9 (SEQ ID NO:1071) also has the following non-silent SNPs (Single Nucleotide Polymorphisms) as listed in Table 42, (given according to their position(s) on the amino acid sequence, with the alternative amino acid(s) listed; the last column indicates whether the SNP is known or not; the presence of known SNPs in variant protein T23657_P9 (SEQ ID NO:1071) sequence provides support for the deduced sequence of this variant protein according to the present invention).

TABLE 42
Amino acid mutations

| SNP position(s) on | Alternative | Previously |
|---|---|---|
| amino acid sequence | amino acid(s) | known SNP? |
| 63 | G -> V | Yes |
| 86 | G -> R | Yes |
| 197 | G -> R | Yes |
| 345 | R -> | No |
| 345 | R -> K | No |

Variant protein T23657_P9 (SEQ ID NO:1071) is encoded by the following transcript(s): T23657_T14 (SEQ ID NO:1012), for which the sequence(s) is/are given at the end of the application. The coding portion of transcript T23657_T14 (SEQ ID NO:1012) is shown in bold; this coding portion starts at position 573 and ends at position 1772. The transcript also has the following SNPs as listed in Table 43 (given according to their position on the nucleotide sequence, with the alternative nucleic acid listed; the last column indicates whether the SNP is known or not; the presence of known SNPs in variant protein T23657_P9 (SEQ ID NO:1071) sequence provides support for the deduced sequence of this variant protein according to the present invention).

TABLE 43
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1161 | G -> A | Yes |
| 1606 | G -> | No |
| 1606 | G -> A | No |
| 2308 | C -> T | No |
| 2311 | C -> | No |
| 2334 | C -> T | Yes |
| 2610 | C -> T | Yes |
| 2623 | C -> T | Yes |

Variant protein T23657_P10 (SEQ ID NO:1072) according to the present invention has an amino acid sequence as given at the end of the application; it is encoded by transcript(s) T23657_T15 (SEQ ID NO:1013). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P10 (SEQ ID NO:1072) and S21C_HUMAN (SEQ ID NO:1062):

1. An isolated chimeric polypeptide encoding for T23657_P10 (SEQ ID NO:1072), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARG THEVRYVSAGQSVACGWWAFAPPCLQVLNTPKGILFFLCAAAFLQGMTVNGFINTVITSLERRYDLHSYQ SGLIASSYDIAACLCLTFVSYFGGSGHKPRWLGWGVLLMGTGSLVFALPHFTAGRYEVELDAGVRTCPAN PGAVCADSTSGLSRYQLVFMLGQFLHGVGATPLYTLGVTYLDENVKSSCSPVYIAIFYTAAILGPAAGYLI GGALLNIYTEMGRRTELTTESPLWVGAWWVGFLGSGAAAFFTAVPILGYPRQLPGSQRYAVMRAAEMH QLKDSSRGEASNPDFGKTIRDLPLSIWLLLKNPTFILLCLAGATEATLITGMSTFSPKFLESQFSLSASEAATL FGYLVVPAGGGGTFLGGFFVNKLRLRGSAVIKFCLFCTVVSLLGILVFSLHCPSVPMAGVTASYGGSLLPE GHLNLTAPCNAACSCQPEHYSPVCGSDGLMYFSLCHAGCPAATETNVDGQKVYRDCSCIPQNLSSGFGHA TAGKCTSTCQRKPLLLVFIFVVIFFTFLSSIPALTATLRCVRDPQRSFALGIQWIVVRIL corresponding to amino acids 1-625 of S21C_HUMAN (SEQ ID NO:1062), which also corresponds to amino acids 1-625 of T23657_P10 (SEQ ID NO:1072), a second amino acid sequence being at least 70%, optionally at least 80%, preferably at least 85%, more preferably at least 90% and most preferably at least 95% homologous to a polypeptide having the sequence GTVQCEEAMVSCTVCSLHKGM (SEQ ID NO:1574) corresponding to amino acids 626-646 of T23657_P10 (SEQ ID NO:1072), and a third amino acid sequence being at least 90% homologous to GGIPGPIAFGWVIDKACLLWQDQCGQQGSCLVYQNSAMSRYILIMGLLYKVLGVLFFAIACFLYKPLSESS DGLETCLPSQSSAPDSATDSQLQSSV corresponding to amino acids 626-722 of S21C_HUMAN (SEQ ID NO:1062), which also corresponds to amino acids 647-743 of T23657_P10 (SEQ ID NO:1072), wherein said first amino acid sequence, second amino acid sequence and third amino acid sequence are contiguous and in a sequential order.

2. An isolated polypeptide encoding for an edge portion of T23657_P10 (SEQ ID NO:1072), comprising an amino acid sequence being at least 70%, optionally at least about 80%, preferably at least about 85%, more preferably at least about 90% and most preferably at least about 95% homologous to the sequence encoding for GTVQCEEAMVSCTVCSLHKGM (SEQ ID NO:1574), corresponding to T23657_P10 (SEQ ID NO:1072).

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein T23657_P10 (SEQ ID NO:1072) also has the following non-silent SNPs (Single Nucleotide Polymorphisms) as listed in Table 44, (given according to their position(s) on the amino acid sequence, with the alternative amino acid(s) listed; the last column indicates whether the SNP is known or not; the presence of known SNPs in variant protein T23657_P10 (SEQ ID NO:1072) sequence provides support for the deduced sequence of this variant protein according to the present invention).

TABLE 44
Amino acid mutations

| SNP position(s) on | Alternative | Previously |
|---|---|---|
| amino acid sequence | amino acid(s) | known SNP? |
| 78 | V -> I | Yes |
| 206 | R -> K | Yes |
| 496 | G -> | No |
| 496 | G -> S | No |

The glycosylation sites of variant protein T23657_P10 (SEQ ID NO:1072), as compared to the known protein Solute carrier family 21 member 12 (SEQ ID NO:1062), are described in Table 45 (given according to their position(s) on the amino acid sequence in the first column; the second column indicates whether the glycosylation site is present in the variant protein; and the last column indicates whether the position is different on the variant protein).

TABLE 45
Glycosylation site(s)

| Position(s) on known | Present in | Position in |
|---|---|---|
| amino acid sequence | variant protein? | variant protein? |
| 557 | yes | 557 |
| 499 | yes | 499 |

Variant protein T23657_P10 (SEQ ID NO:1072) is encoded by the following transcript(s): T23657_T15 (SEQ ID NO:1013), for which the sequence(s) is/are given at the end of the application. The coding portion of transcript T23657_T15 (SEQ ID NO:1013) is shown in bold; this coding portion starts at position 212 and ends at position 2440. The transcript also has the following SNPs as listed in Table 46 (given according to their position on the nucleotide sequence, with the alternative nucleic acid listed; the last column indicates whether the SNP is known or not; the presence of known SNPs in variant protein T23657_P10 (SEQ ID NO:1072) sequence provides support for the deduced sequence of this variant protein according to the present invention).

TABLE 46
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2462 | C -> T | No |
| 2465 | C -> | No |
| 2488 | C -> T | Yes |
| 2764 | C -> T | Yes |
| 2777 | C -> T | Yes |

Variant protein T23657_P11 (SEQ ID NO:1073) according to the present invention has an amino acid sequence as given at the end of the application; it is encoded by transcript(s) T23657_T23 (SEQ ID NO:1020). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P11 (SEQ ID NO:1073) and S21C_HUMAN (SEQ ID NO:1062):

1. An isolated chimeric polypeptide encoding for T23657_P11 (SEQ ID NO:1073), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARG THEVRYVSAGQSVACGWWAFAPPCLQVLNTPKGILFFLCAAAFLQGMTVNGFINTVITSLERRYDLHSYQ SGLIASSYDIAACLCLTFVSYFGGSGHKPRWLGWGVLLMGTGSLVFALPHFTAGRYEVELDAGVRTCPAN PGAVCADSTSGLSRYQLVFMLGQFLHGVGATPLYTLGVTYLDENVKSSCSPVYIAIFYTAAILGPAAGYLI GGALLNIYTEMGRRTELTTESPLWVGAWWVGFLGSGAAAFFTAVPILGYPRQLPGSQRYAVMRAAEMH QLKDSSRGEASNPDFGKTIRDLPLSIWLLLKNPTFILLCLAGATEATLITGMSTFSPKFLESQFSLSASEAATL F corresponding to amino acids 1-425 of S21C_HUMAN (SEQ ID NO:1062), which also corresponds to amino acids 1-425 of T23657_P11 (SEQ ID NO:1073), and a second amino acid sequence being at least 70%, optionally at least 80%, preferably at least 85%, more preferably at least 90% and most preferably at least 95% homologous to a polypeptide having the sequence ASCPKAT (SEQ ID NO:1460) corresponding to amino acids 426-432 of T23657_P11 (SEQ ID NO:1073), wherein said first amino acid sequence and second amino acid sequence are contiguous and in a sequential order.

2. An isolated polypeptide encoding for a tail of T23657_P11 (SEQ ID NO:1073), comprising a polypeptide being at least 70%, optionally at least about 80%, preferably at least about 85%, more preferably at least about 90% and most preferably at least about 95% homologous to the sequence ASCPKAT (SEQ ID NO:1460) in T23657_P11 (SEQ ID NO:1073).

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein T23657_P11 (SEQ ID NO:1073) also has the following non-silent SNPs (Single Nucleotide Polymorphisms) as listed in Table 47, (given according to their position(s) on the amino acid sequence, with the alternative amino acid(s) listed; the last column indicates whether the SNP is known or not; the presence of known SNPs in variant protein T23657_P11 (SEQ ID NO:1073) sequence provides support for the deduced sequence of this variant protein according to the present invention).

TABLE 47
Amino acid mutations

| SNP position(s) on | Alternative | Previously |
|---|---|---|
| amino acid sequence | amino acid(s) | known SNP? |
| 78 | V -> I | Yes |
| 206 | R -> K | Yes |
| 430 | K -> | No |

The glycosylation sites of variant protein T23657_P11 (SEQ ID NO:1073), as compared to the known protein Solute carrier family 21 member 12 (SEQ ID NO:1062), are described in Table 48 (given according to their position(s) on the amino acid sequence in the first column; the second column indicates whether the glycosylation site is present in the variant protein; and the last column indicates whether the position is different on the variant protein).

TABLE 48
Glycosylation site(s)

| Position(s) on known | Present in | Position in |
|---|---|---|
| amino acid sequence | variant protein? | variant protein? |
| 557 | no | |
| 499 | no | |

Variant protein T23657_P11 (SEQ ID NO:1073) is encoded by the following transcript(s): T23657_T23 (SEQ ID NO:1020), for which the sequence(s) is/are given at the end of the application. The coding portion of transcript T23657_T23 (SEQ ID NO:1020) is shown in bold; this coding portion starts at position 212 and ends at position 1507. The transcript also has the following SNPs as listed in Table 49 (given according to their position on the nucleotide sequence, with the alternative nucleic acid listed; the last column indicates whether the SNP is known or not; the presence of known SNPs in variant protein T23657_P11 (SEQ ID NO:1073) sequence provides support for the deduced sequence of this variant protein according to the present invention).

TABLE 49
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1501 | G -> | No |
| 1501 | G -> A | No |
| 2030 | C -> T | No |
| 2033 | C -> | No |
| 2056 | C -> T | Yes |
| 2332 | C -> T | Yes |
| 2345 | C -> T | Yes |

Variant protein T23657_P12 (SEQ ID NO:1074) according to the present invention has an amino acid sequence as given at the end of the application; it is encoded by transcript(s) T23657_T24 (SEQ ID NO:1021). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P12 (SEQ ID NO:1074) and S21C_HUMAN (SEQ ID NO:1062):

1. An isolated chimeric polypeptide encoding for T23657_P12 (SEQ ID NO:1074), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARG THEVRYVSAGQSVACGWWAFAPPCLQVLNTPKGILFFLCAAAFLQGMTVNGFINTVITSLERRYDLHSYQ SGLIASSYDIAACLCLTFVSYFGGSGHKPRWLGWGVLLMGTGSLVFALPHFTAGRYEVELDAGVRTCPAN PGAVCADSTSGLSRYQLVFMLGQFLHGVGATPLYTLGVTYLDENVKSSCSPVYIAIFYTAAILGPAAGYLI GGALLNIYTEMGRRTELTTESPLWVGAWWVGFLGSGAAAFFTAVPILGYPRQLPGSQRYAVMRAAEMH QLKDSSRGEASNPDFGKTIRDLPLSIWLLLKNPTFILLCLAGATEATLITGMSTFSPKFLESQFSLSASEAATL FGYLVVPAGGGGTFLGGFFVNKLRLRGSAVIKFCLFCTVVSLLGILVFSLHCPSVPMAGVTASYGGSLLPE GHLNLTAPCNAACSCQPEHYSPVCGSDGLMYFSLCHAGCPAATETNVDGQKVYRDCSCIPQNLSSGFGHA TAGKCTSTCQRKPLLLVFIFVVIFFTFLSSIPALTATLRCVRDPQRSFALGIQWIVVRILGGIPGPIAFGWVIDK ACLLWQDQCGQQGSCLVYQNSAMSRYILIMGLLYK corresponding to amino acids 1-675 of S21C_HUMAN (SEQ ID NO:1062), which also corresponds to amino acids 1-675 of T23657_P12 (SEQ ID NO:1074), and a second amino acid sequence being at least 70%, optionally at least 80%, preferably at least 85%, more preferably at least 90% and most preferably at least 95% homologous to a polypeptide having the sequence EEENEFRRL (SEQ ID NO:1461) corresponding to amino acids 676-684 of T23657_P12 (SEQ ID NO:1074), wherein said first amino acid sequence and second amino acid sequence are contiguous and in a sequential order.

2. An isolated polypeptide encoding for a tail of T23657_P12 (SEQ ID NO:1074), comprising a polypeptide being at least 70%, optionally at least about 80%, preferably at least about 85%, more preferably at least about 90% and most preferably at least about 95% homologous to the sequence EEENEFRRL (SEQ ID NO:1461) in T23657_P12 (SEQ ID NO:1074).

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein T23657_P12 (SEQ ID NO:1074) also has the following non-silent SNPs (Single Nucleotide Polymorphisms) as listed in Table 50, (given according to their position(s) on the amino acid sequence, with the alternative amino acid(s) listed; the last column indicates whether the SNP is known or not; the presence of known SNPs in variant protein T23657_P12 (SEQ ID NO:1074) sequence provides support for the deduced sequence of this variant protein according to the present invention).

TABLE 50
Amino acid mutations

| SNP position(s) on | Alternative | Previously |
|---|---|---|
| amino acid sequence | amino acid(s) | known SNP? |
| 78 | V -> I | Yes |
| 206 | R -> K | Yes |
| 496 | G -> | No |
| 496 | G -> S | No |

The glycosylation sites of variant protein T23657_P12 (SEQ ID NO:1074), as compared to the known protein Solute carrier family 21 member 12 (SEQ ID NO:1062), are described in Table 51 (given according to their position(s) on the amino acid sequence in the first column; the second column indicates whether the glycosylation site is present in the variant protein; and the last column indicates whether the position is different on the variant protein).

TABLE 51
Glycosylation site(s)

| Position(s) on known | Present in | Position in |
|---|---|---|
| amino acid sequence | variant protein? | variant protein? |
| 557 | yes | 557 |
| 499 | yes | 499 |

Variant protein T23657_P12 (SEQ ID NO:1074) is encoded by the following transcript(s): T23657_T24 (SEQ ID NO:1021), for which the sequence(s) is/are given at the end of the application. The coding portion of transcript T23657_T24 (SEQ ID NO:1021) is shown in bold; this coding portion starts at position 212 and ends at position 2263. The transcript also has the following SNPs as listed in Table 52 (given according to their position on the nucleotide sequence, with the alternative nucleic acid listed; the last column indicates whether the SNP is known or not; the presence of known SNPs in variant protein T23657_P12 (SEQ ID NO:1074) sequence provides support for the deduced sequence of this variant protein according to the present invention).

TABLE 52
Nucleic acid SNPs

| SNP position on | Alternative | Previously |
|---|---|---|
| nucleotide sequence | nucleic acid | known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 2451 | C -> T | Yes |
| 2585 | G -> A | Yes |

Variant protein T23657_P16 (SEQ ID NO:1075) according to the present invention has an amino acid sequence as given at the end of the application; it is encoded by transcript(s) T23657_T30 (SEQ ID NO:1023). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P16 (SEQ ID NO:1075) and S21C_HUMAN (SEQ ID NO:1062):

1. An isolated chimeric polypeptide encoding for T23657_P16 (SEQ ID NO:1075), comprising a first amino acid sequence being at least 70%, optionally at least 80%, preferably at least 85%, more preferably at least 90% and most preferably at least 95% homologous to a polypeptide having the sequence MGTSPMADPVPAGRQHGSGLDPTTRLSPLC corresponding to amino acids 1-30 of T23657_P16 (SEQ ID NO:1075), and a second amino acid sequence being at least 90% homologous to SLLPEGHLNLTAPCNAACSCQPEHYSPVCGSDGLMYFSLCHAGCPAATETNVDGQKVYRDCSCIPQNLSS GFGHATAGKCTSTCQRKPLLLVFIFVVIFFTFLSSIPALTATLRCVRDPQRSFALGIQWIVVRILGGIPGPIAFG WVIDKACLLWQDQCGQQGSCLVYQNSAMSRYILIMGLLYKVLGVLFFAIACFLYKPLSESSDGLETCLPS QSSAPDSATDSQLQSSV corresponding to amino acids 491-722 of S21C_HUMAN (SEQ ID NO:1062), which also corresponds to amino acids 31-262 of T23657_P16 (SEQ ID NO:1075), wherein said first amino acid sequence and second amino acid sequence are contiguous and in a sequential order.

2. An isolated polypeptide encoding for a head of T23657_P16 (SEQ ID NO:1075), comprising a polypeptide being at least 70%, optionally at least about 80%, preferably at least about 85%, more preferably at least about 90% and most preferably at least about 95% homologous to the sequence MGTSPMADPVPAGRQHGSGLDPTTRLSPLC of T23657_P16 (SEQ ID NO:1075).

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein T23657_P16 (SEQ ID NO:1075) also has the following non-silent SNPs (Single Nucleotide Polymorphisms) as listed in Table 53, (given according to their position(s) on the amino acid sequence, with the alternative amino acid(s) listed; the last column indicates whether the SNP is known or not; the presence of known SNPs in variant protein T23657_P16 (SEQ ID NO:1075) sequence provides support for the deduced sequence of this variant protein according to the present invention).

TABLE 53
Amino acid mutations

| SNP position(s) on | Alternative | Previously |
|---|---|---|
| amino acid sequence | amino acid(s) | known SNP? |
| 36 | G -> | No |
| 36 | G -> S | No |

The glycosylation sites of variant protein T23657_P16 (SEQ ID NO:1075), as compared to the known protein Solute carrier family 21 member 12 (SEQ ID NO:1062), are described in Table 54 (given according to their position(s) on the amino acid sequence in the first column; the second column indicates whether the glycosylation site is present in the variant protein; and the last column indicates whether the position is different on the variant protein).

TABLE 54
Glycosylation site(s)

| Position(s) on known | Present in | Position in |
|---|---|---|
| amino acid sequence | variant protein? | variant protein? |
| 557 | yes | 97 |
| 499 | yes | 39 |

Variant protein T23657_P16 (SEQ ID NO:1075) is encoded by the following transcript(s): T23657_T30 (SEQ ID NO:1023), for which the sequence(s) is/are given at the end of the application. The coding portion of transcript T23657_T30 (SEQ ID NO:1023) is shown in bold; this coding portion starts at position 184 and ends at position 969. The transcript also has the following SNPs as listed in Table 55 (given according to their position on the nucleotide sequence, with the alternative nucleic acid listed; the last column indicates whether the SNP is known or not; the presence of known SNPs in variant protein T23657_P16 (SEQ ID NO:1075) sequence provides support for the deduced sequence of this variant protein according to the present invention).

TABLE 55
Nucleic acid SNPs

| SNP position on | Alternative | Previously |
|---|---|---|
| nucleotide sequence | nucleic acid | known SNP? |
| 35 | G -> A | Yes |
| 38 | G -> A | Yes |
| 145 | G -> T | Yes |
| 146 | C -> G | Yes |
| 289 | G -> | No |
| 289 | G -> A | No |
| 991 | C -> T | No |
| 994 | C -> | No |
| 1017 | C -> T | Yes |
| 1293 | C -> T | Yes |
| 1306 | C -> T | Yes |

Variant protein T23657_P17 (SEQ ID NO:1076) according to the present invention has an amino acid sequence as given at the end of the application; it is encoded by transcript(s) T23657_T31 (SEQ ID NO:1024) and T23657_T32 (SEQ ID NO:1025). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P17 (SEQ ID NO:1076) and S21C_HUMAN (SEQ ID NO:1062):

1. An isolated chimeric polypeptide encoding for T23657_P17 (SEQ ID NO:1076), comprising a first amino acid sequence being at least 90% homologous to MYFSLCHAGCPAATETNVDGQKVYRDCSCIPQNLSSGFGHATAGKCTSTCQRKPLLLVFIFVVIFFTFLSSIP ALTATLRCVRDPQRSFALGIQWIVVRILGGIPGPIAFGWVIDKACLLWQDQCGQQGSCLVYQNSAMSRYIL IMGLLYKVLGVLFFAIACFLYKPLSESSDGLETCLPSQSSAPDSATDSQLQSSV corresponding to amino acids 525-722 of S21C_HUMAN (SEQ ID NO:1062), which also corresponds to amino acids 1-198 of T23657_P17 (SEQ ID NO:1076).

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

The glycosylation sites of variant protein T23657_P17 (SEQ ID NO:1076), as compared to the known protein Solute carrier family 21 member 12 (SEQ ID NO:1062), are described in Table 56 (given according to their position(s) on the amino acid sequence in the first column; the second column indicates whether the glycosylation site is present in the variant protein; and the last column indicates whether the position is different on the variant protein).

TABLE 56
Glycosylation site(s)

| Position(s) on known | Present in | Position in |
|---|---|---|
| amino acid sequence | variant protein? | variant protein? |
| 557 | yes | 33 |
| 499 | no | |

Variant protein T23657_P17 (SEQ ID NO:1076) is encoded by the following transcript(s): T23657_T31 (SEQ ID NO:1024) and T23657_T32 (SEQ ID NO:1025), for which the sequence(s) is/are given at the end of the application.

The coding portion of transcript T23657_T31 (SEQ ID NO:1024) is shown in bold; this coding portion starts at position 216 and ends at position 809. The transcript also has the following SNPs as listed in Table 57 (given according to their position on the nucleotide sequence, with the alternative nucleic acid listed; the last column indicates whether the SNP is known or not; the presence of known SNPs in variant protein T23657_P17 (SEQ ID NO:1076) sequence provides support for the deduced sequence of this variant protein according to the present invention).

TABLE 57
Nucleic acid SNPs

| SNP position on nucleotide | Alternative | |
|---|---|---|
| sequence | nucleic acid | Previously known SNP? |
| 129 | G -> | No |
| 129 | G -> A | No |
| 831 | C -> T | No |
| 834 | C -> | No |
| 857 | C -> T | Yes |
| 1133 | C -> T | Yes |
| 1146 | C -> T | Yes |

The coding portion of transcript T23657_T32 (SEQ ID NO:1025) is shown in bold; this coding portion starts at position 174 and ends at position 767. The transcript also has the following SNPs as listed in Table 58 (given according to their position on the nucleotide sequence, with the alternative nucleic acid listed; the last column indicates whether the SNP is known or not; the presence of known SNPs in variant protein T23657_P17 (SEQ ID NO:1076) sequence provides support for the deduced sequence of this variant protein according to the present invention).

TABLE 58
Nucleic acid SNPs

| SNP position on nucleotide | Alternative | |
|---|---|---|
| sequence | nucleic acid | Previously known SNP? |
| 87 | G -> | No |
| 87 | G -> A | No |
| 789 | C -> T | No |
| 792 | C -> | No |
| 815 | C -> T | Yes |
| 1091 | C -> T | Yes |
| 1104 | C -> T | Yes |

Variant protein T23657_P19 (SEQ ID NO:1077) according to the present invention has an amino acid sequence as given at the end of the application; it is encoded by transcript(s) T23657_T35 (SEQ ID NO:1026). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein T23657_P19 (SEQ ID NO:1077) also has the following non-silent SNPs (Single Nucleotide Polymorphisms) as listed in Table 59, (given according to their position(s) on the amino acid sequence, with the alternative amino acid(s) listed; the last column indicates whether the SNP is known or not; the presence of known SNPs in variant protein T23657_P19 (SEQ ID NO:1077) sequence provides support for the deduced sequence of this variant protein according to the present invention).

TABLE 59
Amino acid mutations

| SNP position(s) on amino acid | Alternative | |
|---|---|---|
| sequence | amino acid(s) | Previously known SNP? |
| 36 | G -> | No |
| 36 | G -> S | No |
| 113 | G -> R | Yes |

Variant protein T23657_P19 (SEQ ID NO:1077) is encoded by the following transcript(s): T23657_T35 (SEQ ID NO:1026), for which the sequence(s) is/are given at the end of the application. The coding portion of transcript T23657_T35 (SEQ ID NO:1026) is shown in bold; this coding portion starts at position 184 and ends at position 663. The transcript also has the following SNPs as listed in Table 60 (given according to their position on the nucleotide sequence, with the alternative nucleic acid listed; the last column indicates whether the SNP is known or not; the presence of known SNPs in variant protein T23657_P19 (SEQ ID NO:1077) sequence provides support for the deduced sequence of this variant protein according to the present invention).

TABLE 60
Nucleic acid SNPs

| SNP position on nucleotide | Alternative | |
|---|---|---|
| sequence | nucleic acid | Previously known SNP? |
| 35 | G -> A | Yes |
| 38 | G -> A | Yes |
| 145 | G -> T | Yes |
| 146 | C -> G | Yes |
| 289 | G -> | No |
| 289 | G -> A | No |
| 520 | G -> A | Yes |
| 849 | G -> A | Yes |
| 1488 | A -> G | Yes |
| 1774 | G -> A | Yes |
| 2429 | C -> T | Yes |
| 2921 | T -> C | Yes |
| 3181 | C -> T | No |
| 3184 | C -> | No |
| 3207 | C -> T | Yes |
| 3483 | C -> T | Yes |
| 3496 | C -> T | Yes |

Variant protein T23657_P21 (SEQ ID NO:1078) according to the present invention has an amino acid sequence as given at the end of the application; it is encoded by transcript(s) T23657_T37 (SEQ ID NO:1027). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P21 (SEQ ID NO:1078) and S21C_HUMAN:

1. An isolated chimeric polypeptide encoding for T23657_P21 (SEQ ID NO:1078), comprising a first amino acid sequence being at least 70%, optionally at least 80%, preferably at least 85%, more preferably at least 90% and most preferably at least 95% homologous to a polypeptide having the sequence MWTAR corresponding to amino acids 1-5 of T23657_P21 (SEQ ID NO:1078), and a second amino acid sequence being at least 90% homologous to RCVRDPQRSFALGIQWIVVRILGGIPGPIAFGWVIDKACLLWQDQCGQQGSCLVYQNSAMSRYILIMGLLY KVLGVLFFAIACFLYKPLSESSDGLETCLPSQSSAPDSATDSQLQSSV corresponding to amino acids 604-722 of S21C_HUMAN (SEQ ID NO:1062), which also corresponds to amino acids 6-124 of T23657_P21 (SEQ ID NO:1078), wherein said first amino acid sequence and second amino acid sequence are contiguous and in a sequential order.

2. An isolated polypeptide encoding for a head of T23657_P21 (SEQ ID NO:1078), comprising a polypeptide being at least 70%, optionally at least about 80%, preferably at least about 85%, more preferably at least about 90% and most preferably at least about 95% homologous to the sequence MWTAR of T23657_P21 (SEQ ID NO:1078).

The location of the variant protein was determined according to results from a number of different software programs and analyses, including analyses from SignalP and other specialized programs. The variant protein is believed to be located as follows with regard to the cell: membrane. The protein localization is believed to be membrane because although one of the signal-peptide prediction programs predicts that this protein has a signal peptide (HMM: Signal peptide/NN: NO), both trans-membrane region prediction programs predict that this protein has a trans-membrane region downstream of this signal peptide.

The glycosylation sites of variant protein T23657_P21 (SEQ ID NO:1078), as compared to the known protein Solute carrier family 21 member 12 (SEQ ID NO:1062), are described in Table 61 (given according to their position(s) on the amino acid sequence in the first column; the second column indicates whether the glycosylation site is present in the variant protein; and the last column indicates whether the position is different on the variant protein).

TABLE 61
Glycosylation site(s)

| Position(s) on known amino acid | Present in | Position in |
|---|---|---|
| sequence | variant protein? | variant protein? |
| 557 | no | |
| 499 | no | |

Variant protein T23657_P21 (SEQ ID NO:1078) is encoded by the following transcript(s): T23657_T37 (SEQ ID NO:1027), for which the sequence(s) is/are given at the end of the application. The coding portion of transcript T23657_T37 (SEQ ID NO:1027) is shown in bold; this coding portion starts at position 223 and ends at position 594. The transcript also has the following SNPs as listed in Table 62 (given according to their position on the nucleotide sequence, with the alternative nucleic acid listed; the last column indicates whether the SNP is known or not; the presence of known SNPs in variant protein T23657_P21 (SEQ ID NO:1078) sequence provides support for the deduced sequence of this variant protein according to the present invention).

TABLE 62
Nucleic acid SNPs

| SNP position on nucleotide | Alternative | |
|---|---|---|
| sequence | nucleic acid | Previously known SNP? |
| 87 | G -> | No |
| 87 | G -> A | No |
| 616 | C -> T | No |
| 619 | C -> | No |
| 642 | C -> T | Yes |
| 918 | C -> T | Yes |
| 931 | C -> T | Yes |

Variant protein T23657_P22 (SEQ ID NO:1079) according to the present invention has an amino acid sequence as given at the end of the application; it is encoded by transcript(s) T23657_T38 (SEQ ID NO:1028). The location of the variant protein was determined according to results from a number of different software programs and analyses, including analyses from SignalP and other specialized programs. The variant protein is believed to be located as follows with regard to the cell: unknown.

Variant protein T23657_P22 (SEQ ID NO:1079) also has the following non-silent SNPs (Single Nucleotide Polymorphisms) as listed in Table 63, (given according to their position(s) on the amino acid sequence, with the alternative amino acid(s) listed; the last column indicates whether the SNP is known or not; the presence of known SNPs in variant protein T23657_P22 (SEQ ID NO:1079) sequence provides support for the deduced sequence of this variant protein according to the present invention).

TABLE 63
Amino acid mutations

| SNP position(s) on amino acid | Alternative | |
|---|---|---|
| sequence | amino acid(s) | Previously known SNP? |
| 119 | L -> P | Yes |
| 125 | T -> P | Yes |

Variant protein T23657_P22 (SEQ ID NO:1079) is encoded by the following transcript(s): T23657_T38 (SEQ ID NO:1028), for which the sequence(s) is/are given at the end of the application. The coding portion of transcript T23657_T38 (SEQ ID NO:1028) is shown in bold; this coding portion starts at position 55 and ends at position 88889. The transcript also has the following SNPs as listed in Table 64 (given according to their position on the nucleotide sequence, with the alternative nucleic acid listed; the last column indicates whether the SNP is known or not; the presence of known SNPs in variant protein T23657_P22 (SEQ ID NO:1079) sequence provides support for the deduced sequence of this variant protein according to the present invention).

TABLE 64
Nucleic acid SNPs

| SNP position on nucleotide | Alternative | |
|---|---|---|
| sequence | nucleic acid | Previously known SNP? |
| 35 | G -> A | Yes |
| 38 | G -> A | Yes |
| 410 | T -> C | Yes |
| 427 | A -> C | Yes |

Variant protein T23657_P23 (SEQ ID NO:1080) according to the present invention has an amino acid sequence as given at the end of the application; it is encoded by transcript(s) T23657_T11 (SEQ ID NO:1009). An alignment is given to the known protein (Solute carrier family 21 member 12 (SEQ ID NO:1062)) at the end of the application. One or more alignments to one or more previously published protein sequences are given at the end of the application. A brief description of the relationship of the variant protein according to the present invention to each such aligned protein is as follows:

Comparison Report Between T23657_P23 (SEQ ID NO:1080) and S21C_HUMAN (SEQ ID NO:1062):

1. An isolated chimeric polypeptide encoding for T23657_P23 (SEQ ID NO:1080), comprising a first amino acid sequence being at least 90% homologous to MPLHQLGDKPLTFPSPNSAMENGLDHTPPSRRASPGTPLSPGSLRSAAHSPLDTSKQPLCQLWAEKHGARG THEVRYVSAGQSVACGWWAFAPPCLQVLNTPKGILFFLCAAAFLQGMTVNGFINTVITSLERRYDLHSYQ SGLIASSYDIAACLCLTFVSYFGGSGHKPRWLGWGVLLMGTGSLVFALPHFTAGRYEVELDAGVRTCPAN PGAVCADSTSGLSRYQLVFMLGQFLHGVGATPLYTLGVTYLDENVKSSCSPVYIAIFYTAAILGPAAGYLI GGALLNIYTEMGRRTELTTESPLWVGAWWVGFLGSGAAAFFTAVPILGYPRQLPGSQRYAVMRAAEMH QLKDSSRGEASNPDFGKTIRDLPLSIWLLLKNPTFILLCLAGATEATLITGMSTFSPKFLESQFSLSASEAATL FGYLVVPAGGGGTFLGGFFVNKLRLRGSAVIKFCLFCTVVSLLGILVFSLHCPSVPMAGVTASYGGSLLPE GHLNLTAPCNAACSCQPEHYSPVCGSDGLMYFSLCHAGCPAATETNVDGQKV corresponding to amino acids 1-547 of S21C_HUMAN (SEQ ID NO:1062), which also corresponds to amino acids 1-547 of T23657_P23 (SEQ ID NO:1080), and a second amino acid sequence being at least 70%, optionally at least 80%, preferably at least 85%, more preferably at least 90% and most preferably at least 95% homologous to a polypeptide having the sequence SGAAAYRPCPPLDPGKGPPCLPLVIGAIVGLPRCTETVAVSLRIFPLVLAMHCREMHFNLSEKAPPSGFHIR CNFLYIPQQHSCTNGNSTVSWGRVCACPELSLQHPEAELCRS (SEQ ID NO:1464) corresponding to amino acids 548-661 of T23657_P23 (SEQ ID NO:1080), wherein said first amino acid sequence and second amino acid sequence are contiguous and in a sequential order.

2. An isolated polypeptide encoding for a tail of T23657_P23 (SEQ ID NO:1080), comprising a polypeptide being at least 70%, optionally at least about 80%, preferably at least about 85%, more preferably at least about 90% and most preferably at least about 95% homologous to the sequence SGAAAYRPCPPLDPGKGPPCLPLVIGAIVGLPRCTETVAVSLRIFPLVLAMHCREMHFNLSEKAPPSGFHIR CNFLYIPQQHSCTNGNSTVSWGRVCACPELSLQHPEAELCRS (SEQ ID NO:1464) in T23657_P23 (SEQ ID NO:1080).

The location of the variant protein was determined according to results from a number of different software programs and analyses, including analyses from SignalP and other specialized programs. The variant protein is believed to be located as follows with regard to the cell: membrane. The protein localization is believed to be membrane because both trans-membrane region prediction programs predicted a trans-membrane region for this protein.

Variant protein T23657_P23 (SEQ ID NO:1080) also has the following non-silent SNPs (Single Nucleotide Polymorphisms) as listed in Table 65, (given according to their position(s) on the amino acid sequence, with the alternative amino acid(s) listed; the last column indicates whether the SNP is known or not; the presence of known SNPs in variant protein T23657_P23 (SEQ ID NO:1080) sequence provides support for the deduced sequence of this variant protein according to the present invention).

TABLE 65
Amino acid mutations

| SNP position(s) on amino acid | Alternative | |
|---|---|---|
| sequence | amino acid(s) | Previously known SNP? |
| 78 | V -> I | Yes |
| 206 | R -> K | Yes |
| 496 | G -> | No |
| 496 | G -> S | No |
| 573 | G -> R | Yes |

The glycosylation sites of variant protein T23657_P23 (SEQ ID NO:1080), as compared to the known protein Solute carrier family 21 member 12 (SEQ ID NO:1062), are described in Table 66 (given according to their position(s) on the amino acid sequence in the first column; the second column indicates whether the glycosylation site is present in the variant protein; and the last column indicates whether the position is different on the variant protein).

TABLE 66
Glycosylation site(s)

| Position(s) on known amino acid | Present in | Position in |
|---|---|---|
| sequence | variant protein? | variant protein? |
| 557 | no | |
| 499 | yes | 499 |

Variant protein T23657_P23 (SEQ ID NO:1080) is encoded by the following transcript(s): T23657_T11 (SEQ ID NO:1009), for which the sequence(s) is/are given at the end of the application. The coding portion of transcript T23657_T11 (SEQ ID NO:1009) is shown in bold; this coding portion starts at position 212 and ends at position 2195. The transcript also has the following SNPs as listed in Table 67 (given according to their position on the nucleotide sequence, with the alternative nucleic acid listed; the last column indicates whether the SNP is known or not; the presence of known SNPs in variant protein T23657_P23 (SEQ ID NO:1080) sequence provides support for the deduced sequence of this variant protein according to the present invention).

TABLE 67
Nucleic acid SNPs

| SNP position on nucleotide | Alternative | |
|---|---|---|
| sequence | nucleic acid | Previously known SNP? |
| 132 | G -> A | Yes |
| 179 | G -> | No |
| 180 | A -> T | No |
| 443 | G -> A | Yes |
| 760 | G -> T | Yes |
| 828 | G -> A | Yes |
| 894 | -> G | No |
| 894 | -> T | No |
| 1252 | G -> A | Yes |
| 1697 | G -> | No |
| 1697 | G -> A | No |
| 1928 | G -> A | Yes |
| 2257 | G -> A | Yes |
| 2792 | C -> T | No |
| 2795 | C -> | No |
| 2818 | C -> T | Yes |
| 3094 | C -> T | Yes |
| 3107 | C -> T | Yes |

As noted above, cluster T23657 features 33 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster T23657_node_{—}2 (SEQ ID NO:1029) according to the present invention is supported by 30 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1001), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T24 (SEQ ID NO:1021) and T23657_T28 (SEQ ID NO:1022). Table 68 below describes the starting and ending position of this segment on each transcript.

TABLE 68
Segment location on transcripts

| | Segment | Segment |
|---|---|---|
| Transcript name | starting position | ending position |
| T23657_T0 (SEQ ID NO: 998) | 116 | 292 |
| T23657_T1 (SEQ ID NO: 999) | 116 | 292 |
| T23657_T2 (SEQ ID NO: 1000) | 116 | 292 |
| T23657_T3 (SEQ ID NO: 1001) | 116 | 292 |
| T23657_T4 (SEQ ID NO: 1002) | 116 | 292 |
| T23657_T5 (SEQ ID NO: 1003) | 116 | 292 |
| T23657_T6 (SEQ ID NO: 1004) | 116 | 292 |
| T23657_T7 (SEQ ID NO: 1005) | 116 | 292 |
| T23657_T8 (SEQ ID NO: 1006) | 116 | 292 |
| T23657_T9 (SEQ ID NO: 1007) | 116 | 292 |
| T23657_T10 (SEQ ID NO: 1008) | 116 | 292 |
| T23657_T11 (SEQ ID NO: 1009) | 116 | 292 |
| T23657_T12 (SEQ ID NO: 1010) | 116 | 292 |
| T23657_T13 (SEQ ID NO: 1011) | 116 | 292 |
| T23657_T14 (SEQ ID NO: 1012) | 116 | 292 |
| T23657_T15 (SEQ ID NO: 1013) | 116 | 292 |
| T23657_T16 (SEQ ID NO: 1014) | 116 | 292 |
| T23657_T17 (SEQ ID NO: 1015) | 116 | 292 |
| T23657_T19 (SEQ ID NO: 1016) | 116 | 292 |
| T23657_T20 (SEQ ID NO: 1017) | 116 | 292 |
| T23657_T21 (SEQ ID NO: 1018) | 116 | 292 |
| T23657_T22 (SEQ ID NO: 1019) | 116 | 292 |
| T23657_T23 (SEQ ID NO: 1020) | 116 | 292 |
| T23657_T24 (SEQ ID NO: 1021) | 116 | 292 |
| T23657_T28 (SEQ ID NO: 1022) | 116 | 292 |

Segment cluster T23657_node_{—}3 (SEQ ID NO:1030) according to the present invention is supported by 54 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T24 (SEQ ID NO:1021) and T23657_T28 (SEQ ID NO:1022). Table 69 below describes the starting and ending position of this segment on each transcript.

TABLE 69
Segment location on transcripts

| | Segment | Segment |
|---|---|---|
| Transcript name | starting position | ending position |
| T23657_T0 (SEQ ID NO: 998) | 293 | 938 |
| T23657_T1 (SEQ ID NO: 999) | 293 | 938 |
| T23657_T2 (SEQ ID NO: 1000) | 293 | 938 |
| T23657_T3 (SEQ ID NO: 1001) | 293 | 938 |
| T23657_T4 (SEQ ID NO: 1002) | 293 | 938 |
| T23657_T5 (SEQ ID NO: 1003) | 293 | 938 |
| T23657_T6 (SEQ ID NO: 1004) | 293 | 938 |
| T23657_T7 (SEQ ID NO: 1005) | 293 | 938 |
| T23657_T8 (SEQ ID NO: 1006) | 293 | 938 |
| T23657_T9 (SEQ ID NO: 1007) | 293 | 938 |
| T23657_T10 (SEQ ID NO: 1008) | 293 | 938 |
| T23657_T11 (SEQ ID NO: 1009) | 293 | 938 |
| T23657_T12 (SEQ ID NO: 1010) | 293 | 938 |
| T23657_T13 (SEQ ID NO: 1011) | 293 | 938 |
| T23657_T14 (SEQ ID NO: 1012) | 293 | 938 |
| T23657_T15 (SEQ ID NO: 1013) | 293 | 938 |
| T23657_T16 (SEQ ID NO: 1014) | 293 | 938 |
| T23657_T17 (SEQ ID NO: 1015) | 293 | 938 |
| T23657_T19 (SEQ ID NO: 1016) | 293 | 938 |
| T23657_T20 (SEQ ID NO: 1017) | 293 | 938 |
| T23657_T21 (SEQ ID NO: 1018) | 293 | 938 |
| T23657_T22 (SEQ ID NO: 1019) | 293 | 938 |
| T23657_T23 (SEQ ID NO: 1020) | 293 | 938 |
| T23657_T24 (SEQ ID NO: 1021) | 293 | 938 |
| T23657_T28 (SEQ ID NO: 1022) | 293 | 938 |

Segment cluster T23657_node_{—}8 (SEQ ID NO:1031) according to the present invention is supported by 25 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T24 (SEQ ID NO:1021) and T23657_T28 (SEQ ID NO:1022). Table 70 below describes the starting and ending position of this segment on each transcript.

TABLE 70
Segment location on transcripts

| | Segment | Segment |
|---|---|---|
| Transcript name | starting position | ending position |
| T23657_T0 (SEQ ID NO: 998) | 1099 | 1220 |
| T23657_T1 (SEQ ID NO: 999) | 1099 | 1220 |
| T23657_T2 (SEQ ID NO: 1000) | 1099 | 1220 |
| T23657_T3 (SEQ ID NO: 1001) | 1099 | 1220 |
| T23657_T4 (SEQ ID NO: 1002) | 1099 | 1220 |
| T23657_T5 (SEQ ID NO: 1003) | 1099 | 1220 |
| T23657_T6 (SEQ ID NO: 1004) | 1099 | 1220 |
| T23657_T7 (SEQ ID NO: 1005) | 1099 | 1220 |
| T23657_T8 (SEQ ID NO: 1006) | 1099 | 1220 |
| T23657_T9 (SEQ ID NO: 1007) | 1099 | 1220 |
| T23657_T10 (SEQ ID NO: 1008) | 1099 | 1220 |
| T23657_T11 (SEQ ID NO: 1009) | 1099 | 1220 |
| T23657_T12 (SEQ ID NO: 1010) | 1099 | 1220 |
| T23657_T13 (SEQ ID NO: 1011) | 1099 | 1220 |
| T23657_T14 (SEQ ID NO: 1012) | 1008 | 1129 |
| T23657_T15 (SEQ ID NO: 1013) | 1099 | 1220 |
| T23657_T16 (SEQ ID NO: 1014) | 1099 | 1220 |
| T23657_T17 (SEQ ID NO: 1015) | 1099 | 1220 |
| T23657_T19 (SEQ ID NO: 1016) | 1099 | 1220 |
| T23657_T20 (SEQ ID NO: 1017) | 1099 | 1220 |
| T23657_T21 (SEQ ID NO: 1018) | 1099 | 1220 |
| T23657_T22 (SEQ ID NO: 1019) | 1099 | 1220 |
| T23657_T23 (SEQ ID NO: 1020) | 1099 | 1220 |
| T23657_T24 (SEQ ID NO: 1021) | 1099 | 1220 |
| T23657_T28 (SEQ ID NO: 1022) | 1099 | 1220 |

Segment cluster T23657_node_{—}16 (SEQ ID NO:1032) according to the present invention is supported by 39 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T24 (SEQ ID NO:1021) and T23657_T28 (SEQ ID NO:1022). Table 71 below describes the starting and ending position of this segment on each transcript.

TABLE 71
Segment location on transcripts

| | Segment | Segment |
|---|---|---|
| Transcript name | starting position | ending position |
| T23657_T0 (SEQ ID NO: 998) | 1333 | 1487 |
| T23657_T1 (SEQ ID NO: 999) | 1333 | 1487 |
| T23657_T2 (SEQ ID NO: 1000) | 1333 | 1487 |
| T23657_T3 (SEQ ID NO: 1001) | 1333 | 1487 |
| T23657_T4 (SEQ ID NO: 1002) | 1333 | 1487 |
| T23657_T5 (SEQ ID NO: 1003) | 1333 | 1487 |
| T23657_T6 (SEQ ID NO: 1004) | 1333 | 1487 |
| T23657_T7 (SEQ ID NO: 1005) | 1333 | 1487 |
| T23657_T8 (SEQ ID NO: 1006) | 1333 | 1487 |
| T23657_T9 (SEQ ID NO: 1007) | 1333 | 1487 |
| T23657_T10 (SEQ ID NO: 1008) | 1333 | 1487 |
| T23657_T11 (SEQ ID NO: 1009) | 1333 | 1487 |
| T23657_T12 (SEQ ID NO: 1010) | 1333 | 1487 |
| T23657_T13 (SEQ ID NO: 1011) | 1333 | 1487 |
| T23657_T14 (SEQ ID NO: 1012) | 1242 | 1396 |
| T23657_T15 (SEQ ID NO: 1013) | 1333 | 1487 |
| T23657_T16 (SEQ ID NO: 1014) | 1333 | 1487 |
| T23657_T17 (SEQ ID NO: 1015) | 1333 | 1487 |
| T23657_T19 (SEQ ID NO: 1016) | 1333 | 1487 |
| T23657_T20 (SEQ ID NO: 1017) | 1333 | 1487 |
| T23657_T21 (SEQ ID NO: 1018) | 1333 | 1487 |
| T23657_T22 (SEQ ID NO: 1019) | 1333 | 1487 |
| T23657_T23 (SEQ ID NO: 1020) | 1333 | 1487 |
| T23657_T24 (SEQ ID NO: 1021) | 1333 | 1487 |
| T23657_T28 (SEQ ID NO: 1022) | 1333 | 1487 |

Segment cluster T23657_node_{—}18 (SEQ ID NO:1033) according to the present invention is supported by 40 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T24 (SEQ ID NO:1021) and T23657_T28 (SEQ ID NO:1022). Table 72 below describes the starting and ending position of this segment on each transcript.

TABLE 72
Segment location on transcripts

| | Segment | Segment |
|---|---|---|
| Transcript name | starting position | ending position |
| T23657_T0 (SEQ ID NO: 998) | 1488 | 1683 |
| T23657_T1 (SEQ ID NO: 999) | 1488 | 1683 |
| T23657_T2 (SEQ ID NO: 1000) | 1488 | 1683 |
| T23657_T3 (SEQ ID NO: 1001) | 1488 | 1683 |
| T23657_T4 (SEQ ID NO: 1002) | 1488 | 1683 |
| T23657_T5 (SEQ ID NO: 1003) | 1488 | 1683 |
| T23657_T6 (SEQ ID NO: 1004) | 1488 | 1683 |
| T23657_T7 (SEQ ID NO: 1005) | 1488 | 1683 |
| T23657_T8 (SEQ ID NO: 1006) | 1488 | 1683 |
| T23657_T9 (SEQ ID NO: 1007) | 1488 | 1683 |
| T23657_T10 (SEQ ID NO: 1008) | 1488 | 1683 |
| T23657_T11 (SEQ ID NO: 1009) | 1488 | 1683 |
| T23657_T12 (SEQ ID NO: 1010) | 1488 | 1683 |
| T23657_T13 (SEQ ID NO: 1011) | 1488 | 1683 |
| T23657_T14 (SEQ ID NO: 1012) | 1397 | 1592 |
| T23657_T15 (SEQ ID NO: 1013) | 1488 | 1683 |
| T23657_T16 (SEQ ID NO: 1014) | 1488 | 1683 |
| T23657_T17 (SEQ ID NO: 1015) | 1488 | 1683 |
| T23657_T19 (SEQ ID NO: 1016) | 1488 | 1683 |
| T23657_T20 (SEQ ID NO: 1017) | 1488 | 1683 |
| T23657_T21 (SEQ ID NO: 1018) | 1488 | 1683 |
| T23657_T22 (SEQ ID NO: 1019) | 1488 | 1683 |
| T23657_T24 (SEQ ID NO: 1021) | 1488 | 1683 |
| T23657_T28 (SEQ ID NO: 1022) | 1488 | 1683 |

Segment cluster T23657_node_{—}23 (SEQ ID NO:1034) according to the present invention is supported by 4 libraries. The number of libraries was determined as previously described. This segment can be found in the following transcript(s): T23657_T30 (SEQ ID NO:1023) and T23657_T35 (SEQ ID NO:1026). Table 73 below describes the starting and ending position of this segment on each transcript.

TABLE 73
Segment location on transcripts

| | Segment | Segment |
|---|---|---|
| Transcript name | starting position | ending position |
| T23657_T30 (SEQ ID NO: 1023) | 118 | 275 |
| T23657_T35 (SEQ ID NO: 1026) | 118 | 275 |

Segment cluster T23657_node_{—}24 (SEQ ID NO:1035) according to the present invention is supported by 42 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T24 (SEQ ID NO:1021), T23657_T28 (SEQ ID NO:1022), T23657_T30 (SEQ ID NO:1023), T23657_T31 (SEQ ID NO:1024), T23657_T32 (SEQ ID NO:1025), T23657_T35 (SEQ ID NO:1026) and T23657_T37 (SEQ ID NO:1027). Table 74 below describes the starting and ending position of this segment on each transcript.

TABLE 74
Segment location on transcripts

| | Segment | Segment |
|---|---|---|
| Transcript name | starting position | ending position |
| T23657_T0 (SEQ ID NO: 998) | 1684 | 1808 |
| T23657_T1 (SEQ ID NO: 999) | 1684 | 1808 |
| T23657_T2 (SEQ ID NO: 1000) | 1684 | 1808 |
| T23657_T3 (SEQ ID NO: 1001) | 1684 | 1808 |
| T23657_T4 (SEQ ID NO: 1002) | 1684 | 1808 |
| T23657_T5 (SEQ ID NO: 1003) | 1684 | 1808 |
| T23657_T6 (SEQ ID NO: 1004) | 1684 | 1808 |
| T23657_T7 (SEQ ID NO: 1005) | 1684 | 1808 |
| T23657_T8 (SEQ ID NO: 1006) | 1684 | 1808 |
| T23657_T9 (SEQ ID NO: 1007) | 1684 | 1808 |
| T23657_T10 (SEQ ID NO: 1008) | 1684 | 1808 |
| T23657_T11 (SEQ ID NO: 1009) | 1684 | 1808 |
| T23657_T12 (SEQ ID NO: 1010) | 1684 | 1808 |
| T23657_T13 (SEQ ID NO: 1011) | 1684 | 1808 |
| T23657_T14 (SEQ ID NO: 1012) | 1593 | 1717 |
| T23657_T15 (SEQ ID NO: 1013) | 1684 | 1808 |
| T23657_T16 (SEQ ID NO: 1014) | 1684 | 1808 |
| T23657_T17 (SEQ ID NO: 1015) | 1684 | 1808 |
| T23657_T19 (SEQ ID NO: 1016) | 1684 | 1808 |
| T23657_T20 (SEQ ID NO: 1017) | 1684 | 1808 |
| T23657_T21 (SEQ ID NO: 1018) | 1684 | 1808 |
| T23657_T22 (SEQ ID NO: 1019) | 1684 | 1808 |
| T23657_T23 (SEQ ID NO: 1020) | 1488 | 1612 |
| T23657_T24 (SEQ ID NO: 1021) | 1684 | 1808 |
| T23657_T28 (SEQ ID NO: 1022) | 1684 | 1808 |
| T23657_T30 (SEQ ID NO: 1023) | 276 | 400 |
| T23657_T31 (SEQ ID NO: 1024) | 116 | 240 |
| T23657_T32 (SEQ ID NO: 1025) | 74 | 198 |
| T23657_T35 (SEQ ID NO: 1026) | 276 | 400 |
| T23657_T37 (SEQ ID NO: 1027) | 74 | 198 |

Segment cluster T23657_node_{—}27 (SEQ ID NO:1036) according to the present invention is supported by 38 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T14 (SEQ ID NO:1012), T23657_T15 (SEQ ID NO:1013), T23657_T16 (SEQ ID NO:1014), T23657_T20 (SEQ ID NO:1017), T23657_T21 (SEQ ID NO:1018), T23657_T24 (SEQ ID NO:1021), T23657_T30 (SEQ ID NO:1023), T23657_T31 (SEQ ID NO:1024), T23657_T32 (SEQ ID NO:1025) and T23657_T35 (SEQ ID NO:1026). Table 75 below describes the starting and ending position of this segment on each transcript.

TABLE 75
Segment location on transcripts

| | Segment | Segment |
|---|---|---|
| Transcript name | starting position | ending position |
| T23657_T0 (SEQ ID NO: 998) | 1850 | 1992 |
| T23657_T1 (SEQ ID NO: 999) | 1850 | 1992 |
| T23657_T2 (SEQ ID NO: 1000) | 1850 | 1992 |
| T23657_T3 (SEQ ID NO: 1001) | 1850 | 1992 |
| T23657_T4 (SEQ ID NO: 1002) | 1850 | 1992 |
| T23657_T5 (SEQ ID NO: 1003) | 1850 | 1992 |
| T23657_T6 (SEQ ID NO: 1004) | 1850 | 1992 |
| T23657_T7 (SEQ ID NO: 1005) | 1850 | 1992 |
| T23657_T8 (SEQ ID NO: 1006) | 1850 | 1992 |
| T23657_T9 (SEQ ID NO: 1007) | 1850 | 1992 |
| T23657_T10 (SEQ ID NO: 1008) | 1951 | 2093 |
| T23657_T11 (SEQ ID NO: 1009) | 1951 | 2093 |
| T23657_T14 (SEQ ID NO: 1012) | 1759 | 1901 |
| T23657_T15 (SEQ ID NO: 1013) | 1850 | 1992 |
| T23657_T16 (SEQ ID NO: 1014) | 1850 | 1992 |
| T23657_T20 (SEQ ID NO: 1017) | 1850 | 1992 |
| T23657_T21 (SEQ ID NO: 1018) | 1850 | 1992 |
| T23657_T24 (SEQ ID NO: 1021) | 1850 | 1992 |
| T23657_T30 (SEQ ID NO: 1023) | 442 | 584 |
| T23657_T31 (SEQ ID NO: 1024) | 282 | 424 |
| T23657_T32 (SEQ ID NO: 1025) | 240 | 382 |
| T23657_T35 (SEQ ID NO: 1026) | 543 | 685 |

Segment cluster T23657_node_{—}29 (SEQ ID NO:1037) according to the present invention is supported by 12 libraries. The number of libraries was determined as previously described. This segment can be found in the following transcript(s): T23657_T5 (SEQ ID NO:1003), T23657_T6 (SEQ ID NO:1004), T23657_T10 (SEQ ID NO:1008), T23657_T11 (SEQ ID NO:1009) and T23657_T35 (SEQ ID NO:1026). Table 76 below describes the starting and ending position of this segment on each transcript.

TABLE 76
Segment location on transcripts

| | Segment | Segment |
|---|---|---|
| Transcript name | starting position | ending position |
| T23657_T5 (SEQ ID NO: 1003) | 2023 | 2278 |
| T23657_T6 (SEQ ID NO: 1004) | 2023 | 2278 |
| T23657_T10 (SEQ ID NO: 1008) | 2124 | 2379 |
| T23657_T11 (SEQ ID NO: 1009) | 2124 | 2379 |
| T23657_T35 (SEQ ID NO: 1026) | 716 | 971 |

Segment cluster T23657_node_{—}34 (SEQ ID NO:1038) according to the present invention is supported by 65 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T24 (SEQ ID NO:1021), T23657_T28 (SEQ ID NO:1022), T23657_T30 (SEQ ID NO:1023), T23657_T31 (SEQ ID NO:1024), T23657_T32 (SEQ ID NO:1025), T23657_T35 (SEQ ID NO:1026), T23657_T37 (SEQ ID NO:1027) and T23657_T38 (SEQ ID NO:1028). Table 77 below describes the starting and ending position of this segment on each transcript.

TABLE 77
Segment location on transcripts

| | Segment | Segment |
|---|---|---|
| Transcript name | starting position | ending position |
| T23657_T0 (SEQ ID NO: 998) | 2088 | 2236 |
| T23657_T1 (SEQ ID NO: 999) | 2088 | 2236 |
| T23657_T2 (SEQ ID NO: 1000) | 2088 | 2236 |
| T23657_T3 (SEQ ID NO: 1001) | 2088 | 2236 |
| T23657_T4 (SEQ ID NO: 1002) | 2151 | 2299 |
| T23657_T5 (SEQ ID NO: 1003) | 2380 | 2528 |
| T23657_T6 (SEQ ID NO: 1004) | 2443 | 2591 |
| T23657_T7 (SEQ ID NO: 1005) | 2088 | 2236 |
| T23657_T8 (SEQ ID NO: 1006) | 2088 | 2236 |
| T23657_T9 (SEQ ID NO: 1007) | 2088 | 2236 |
| T23657_T10 (SEQ ID NO: 1008) | 2481 | 2629 |
| T23657_T11 (SEQ ID NO: 1009) | 2481 | 2629 |
| T23657_T12 (SEQ ID NO: 1010) | 1915 | 2063 |
| T23657_T13 (SEQ ID NO: 1011) | 1945 | 2093 |
| T23657_T14 (SEQ ID NO: 1012) | 1997 | 2145 |
| T23657_T15 (SEQ ID NO: 1013) | 2151 | 2299 |
| T23657_T16 (SEQ ID NO: 1014) | 2088 | 2236 |
| T23657_T17 (SEQ ID NO: 1015) | 1915 | 2063 |
| T23657_T19 (SEQ ID NO: 1016) | 1945 | 2093 |
| T23657_T20 (SEQ ID NO: 1017) | 2088 | 2236 |
| T23657_T21 (SEQ ID NO: 1018) | 2088 | 2236 |
| T23657_T22 (SEQ ID NO: 1019) | 1915 | 2063 |
| T23657_T23 (SEQ ID NO: 1020) | 1719 | 1867 |
| T23657_T24 (SEQ ID NO: 1021) | 2088 | 2236 |
| T23657_T28 (SEQ ID NO: 1022) | 1945 | 2093 |
| T23657_T30 (SEQ ID NO: 1023) | 680 | 828 |
| T23657_T31 (SEQ ID NO: 1024) | 520 | 668 |
| T23657_T32 (SEQ ID NO: 1025) | 478 | 626 |
| T23657_T35 (SEQ ID NO: 1026) | 1073 | 1221 |
| T23657_T37 (SEQ ID NO: 1027) | 305 | 453 |
| T23657_T38 (SEQ ID NO: 1028) | 254 | 402 |

Segment cluster T23657_node_{—}37 (SEQ ID NO:1039) according to the present invention is supported by 2 libraries. The number of libraries was determined as previously described. This segment can be found in the following transcript(s): T23657_T3 (SEQ ID NO:1001), T23657_T4 (SEQ ID NO:1002), T23657_T6 (SEQ ID NO:1004), T23657_T9 (SEQ ID NO:1007), T23657_T13 (SEQ ID NO:1011) and T23657_T21 (SEQ ID NO:1018). Table 78 below describes the starting and ending position of this segment on each transcript.

TABLE 78
Segment location on transcripts

| | Segment | Segment |
|---|---|---|
| Transcript name | starting position | ending position |
| T23657_T3 (SEQ ID NO: 1001) | 2237 | 2376 |
| T23657_T4 (SEQ ID NO: 1002) | 2300 | 2439 |
| T23657_T6 (SEQ ID NO: 1004) | 2592 | 2731 |
| T23657_T9 (SEQ ID NO: 1007) | 2237 | 2376 |
| T23657_T13 (SEQ ID NO: 1011) | 2094 | 2233 |
| T23657_T21 (SEQ ID NO: 1018) | 2237 | 2376 |

Segment cluster T23657_node_{—}38 (SEQ ID NO:1040) according to the present invention is supported by 2 libraries. The number of libraries was determined as previously described. This segment can be found in the following transcript(s): T23657_T3 (SEQ ID NO:1001), T23657_T4 (SEQ ID NO:1002), T23657_T6 (SEQ ID NO:1004), T23657_T9 (SEQ ID NO:1007) and T23657_T13 (SEQ ID NO:1011). Table 79 below describes the starting and ending position of this segment on each transcript.

TABLE 79
Segment location on transcripts

| | Segment | Segment |
|---|---|---|
| Transcript name | starting position | ending position |
| T23657_T3 (SEQ ID NO: 1001) | 2377 | 2534 |
| T23657_T4 (SEQ ID NO: 1002) | 2440 | 2597 |
| T23657_T6 (SEQ ID NO: 1004) | 2732 | 2889 |
| T23657_T9 (SEQ ID NO: 1007) | 2377 | 2534 |
| T23657_T13 (SEQ ID NO: 1011) | 2234 | 2391 |

Segment cluster T23657_node_{—}39 (SEQ ID NO:1041) according to the present invention is supported by 13 libraries. The number of libraries was determined as previously described. This segment can be found in the following transcript(s): T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T9 (SEQ ID NO:1007), T23657_T10 (SEQ ID NO:1008), T23657_T12 (SEQ ID NO:1010), T23657_T13 (SEQ ID NO:1011), T23657_T16 (SEQ ID NO:1014), T23657_T20 (SEQ ID NO:1017), T23657_T22 (SEQ ID NO:1019) and T23657_T35 (SEQ ID NO:1026). Table 80 below describes the starting and ending position of this segment on each transcript.

TABLE 80
Segment location on transcripts

| | Segment | Segment |
|---|---|---|
| Transcript name | starting position | ending position |
| T23657_T2 (SEQ ID NO: 1000) | 2237 | 2406 |
| T23657_T3 (SEQ ID NO: 1001) | 2535 | 2704 |
| T23657_T4 (SEQ ID NO: 1002) | 2598 | 2767 |
| T23657_T5 (SEQ ID NO: 1003) | 2529 | 2698 |
| T23657_T6 (SEQ ID NO: 1004) | 2890 | 3059 |
| T23657_T7 (SEQ ID NO: 1005) | 2237 | 2406 |
| T23657_T9 (SEQ ID NO: 1007) | 2535 | 2704 |
| T23657_T10 (SEQ ID NO: 1008) | 2630 | 2799 |
| T23657_T12 (SEQ ID NO: 1010) | 2064 | 2233 |
| T23657_T13 (SEQ ID NO: 1011) | 2392 | 2561 |
| T23657_T16 (SEQ ID NO: 1014) | 2237 | 2406 |
| T23657_T20 (SEQ ID NO: 1017) | 2237 | 2406 |
| T23657_T22 (SEQ ID NO: 1019) | 2064 | 2233 |
| T23657_T35 (SEQ ID NO: 1026) | 1222 | 1391 |

Segment cluster T23657_node_{—}40 (SEQ ID NO:1042) according to the present invention is supported by 40 libraries. The number of libraries was determined as previously described. This segment can be found in the following transcript(s): T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T9 (SEQ ID NO:1007), T23657_T10 (SEQ ID NO:1008), T23657_T12 (SEQ ID NO:1010), T23657_T13 (SEQ ID NO:1011), T23657_T16 (SEQ ID NO:1014) and T23657_T35 (SEQ ID NO:1026). Table 81 below describes the starting and ending position of this segment on each transcript.

TABLE 81
Segment location on transcripts

| | Segment | Segment |
|---|---|---|
| Transcript name | starting position | ending position |
| T23657_T2 (SEQ ID NO: 1000) | 2407 | 3973 |
| T23657_T3 (SEQ ID NO: 1001) | 2705 | 4271 |
| T23657_T4 (SEQ ID NO: 1002) | 2768 | 4334 |
| T23657_T5 (SEQ ID NO: 1003) | 2699 | 4265 |
| T23657_T6 (SEQ ID NO: 1004) | 3060 | 4626 |
| T23657_T7 (SEQ ID NO: 1005) | 2407 | 3973 |
| T23657_T9 (SEQ ID NO: 1007) | 2705 | 4271 |
| T23657_T10 (SEQ ID NO: 1008) | 2800 | 4366 |
| T23657_T12 (SEQ ID NO: 1010) | 2234 | 3800 |
| T23657_T13 (SEQ ID NO: 1011) | 2562 | 4128 |
| T23657_T16 (SEQ ID NO: 1014) | 2407 | 3973 |
| T23657_T35 (SEQ ID NO: 1026) | 1392 | 2958 |

Segment cluster T23657_node_{—}45 (SEQ ID NO:1043) according to the present invention is supported by 91 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T30 (SEQ ID NO:1023), T23657_T31 (SEQ ID NO:1024), T23657_T32 (SEQ ID NO:1025), T23657_T35 (SEQ ID NO:1026) and T23657_T37 (SEQ ID NO:1027). Table 82 below describes the starting and ending position of this segment on each transcript.

TABLE 82
Segment location on transcripts

| | Segment | Segment |
|---|---|---|
| Transcript name | starting position | ending position |
| T23657_T0 (SEQ ID NO: 998) | 2363 | 2789 |
| T23657_T1 (SEQ ID NO: 999) | 2363 | 2942 |
| T23657_T2 (SEQ ID NO: 1000) | 4160 | 4586 |
| T23657_T3 (SEQ ID NO: 1001) | 4458 | 4884 |
| T23657_T4 (SEQ ID NO: 1002) | 4521 | 4947 |
| T23657_T5 (SEQ ID NO: 1003) | 4452 | 4878 |
| T23657_T6 (SEQ ID NO: 1004) | 4813 | 5239 |
| T23657_T7 (SEQ ID NO: 1005) | 4160 | 4739 |
| T23657_T8 (SEQ ID NO: 1006) | 2363 | 2594 |
| T23657_T9 (SEQ ID NO: 1007) | 4458 | 4689 |
| T23657_T10 (SEQ ID NO: 1008) | 4553 | 4979 |
| T23657_T11 (SEQ ID NO: 1009) | 2756 | 3182 |
| T23657_T12 (SEQ ID NO: 1010) | 3987 | 4413 |
| T23657_T13 (SEQ ID NO: 1011) | 4315 | 4741 |
| T23657_T14 (SEQ ID NO: 1012) | 2272 | 2698 |
| T23657_T15 (SEQ ID NO: 1013) | 2426 | 2852 |
| T23657_T16 (SEQ ID NO: 1014) | 4067 | 4493 |
| T23657_T17 (SEQ ID NO: 1015) | 2190 | 2616 |
| T23657_T19 (SEQ ID NO: 1016) | 2220 | 2646 |
| T23657_T20 (SEQ ID NO: 1017) | 2533 | 2959 |
| T23657_T21 (SEQ ID NO: 1018) | 2503 | 2929 |
| T23657_T22 (SEQ ID NO: 1019) | 2360 | 2786 |
| T23657_T23 (SEQ ID NO: 1020) | 1994 | 2420 |
| T23657_T30 (SEQ ID NO: 1023) | 955 | 1381 |
| T23657_T31 (SEQ ID NO: 1024) | 795 | 1221 |
| T23657_T32 (SEQ ID NO: 1025) | 753 | 1179 |
| T23657_T35 (SEQ ID NO: 1026) | 3145 | 3571 |
| T23657_T37 (SEQ ID NO: 1027) | 580 | 1006 |

Segment cluster T23657_node_{—}46 (SEQ ID NO:1044) according to the present invention is supported by 7 libraries. The number of libraries was determined as previously described. This segment can be found in the following transcript(s): T23657_T1 (SEQ ID NO:999), T23657_T7 (SEQ ID NO:1005) and T23657_T38 (SEQ ID NO:1028). Table 83 below describes the starting and ending position of this segment on each transcript.

TABLE 83
Segment location on transcripts

| | Segment | Segment |
|---|---|---|
| Transcript name | starting position | ending position |
| T23657_T1 (SEQ ID NO: 999) | 2943 | 3109 |
| T23657_T7 (SEQ ID NO: 1005) | 4740 | 4906 |
| T23657_T38 (SEQ ID NO: 1028) | 403 | 569 |

Segment cluster T23657_node_{—}49 (SEQ ID NO:1045) according to the present invention is supported by 5 libraries. The number of libraries was determined as previously described. This segment can be found in the following transcript(s): T23657_T24 (SEQ ID NO:1021) and T23657_T28 (SEQ ID NO:1022). Table 84 below describes the starting and ending position of this segment on each transcript.

TABLE 84
Segment location on transcripts

| | Segment | Segment |
|---|---|---|
| Transcript name | starting position | ending position |
| T23657_T24 (SEQ ID NO: 1021) | 2237 | 2587 |
| T23657_T28 (SEQ ID NO: 1022) | 2094 | 2444 |

According to an optional embodiment of the present invention, short segments related to the above cluster are also provided. These segments are up to about 120 bp in length, and so are included in a separate description.

Segment cluster T23657_node_{—}0 (SEQ ID NO:1046) according to the present invention is supported by 24 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1000), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T24 (SEQ ID NO:1021), T23657_T28 (SEQ ID NO:1022) and T23657_T31 (SEQ ID NO:1024). Table 85 below describes the starting and ending position of this segment on each transcript.

TABLE 85
Segment location on transcripts

| | Segment | Segment |
|---|---|---|
| Transcript name | starting position | ending position |
| T23657_T0 (SEQ ID NO: 998) | 1 | 115 |
| T23657_T1 (SEQ ID NO: 999) | 1 | 115 |
| T23657_T2 (SEQ ID NO: 1000) | 1 | 115 |
| T23657_T3 (SEQ ID NO: 1001) | 1 | 115 |
| T23657_T4 (SEQ ID NO: 1002) | 1 | 115 |
| T23657_T5 (SEQ ID NO: 1003) | 1 | 115 |
| T23657_T6 (SEQ ID NO: 1004) | 1 | 115 |
| T23657_T7 (SEQ ID NO: 1005) | 1 | 115 |
| T23657_T8 (SEQ ID NO: 1006) | 1 | 115 |
| T23657_T9 (SEQ ID NO: 1007) | 1 | 115 |
| T23657_T10 (SEQ ID NO: 1008) | 1 | 115 |
| T23657_T11 (SEQ ID NO: 1009) | 1 | 115 |
| T23657_T12 (SEQ ID NO: 1010) | 1 | 115 |
| T23657_T13 (SEQ ID NQ: 1011) | 1 | 115 |
| T23657_T14 (SEQ ID NO: 1012) | 1 | 115 |
| T23657_T15 (SEQ ID NO: 1013) | 1 | 115 |
| T23657_T16 (SEQ ID NO: 1014) | 1 | 115 |
| T23657_T17 (SEQ ID NO: 1015) | 1 | 115 |
| T23657_T19 (SEQ ID NO: 1016) | 1 | 115 |
| T23657_T20 (SEQ ID NO: 1017) | 1 | 115 |
| T23657_T21 (SEQ ID NO: 1018) | 1 | 115 |
| T23657_T22 (SEQ ID NO: 1019) | 1 | 115 |
| T23657_T23 (SEQ ID NO: 1020) | 1 | 115 |
| T23657_T24 (SEQ ID NO: 1021) | 1 | 115 |
| T23657_T28 (SEQ ID NO: 1022) | 1 | 115 |
| T23657_T31 (SEQ ID NO: 1024) | 1 | 115 |

Segment cluster T23657_node_{—}4 (SEQ ID NO:1047) according to the present invention is supported by 31 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T24 (SEQ ID NO:1021) and T23657_T28 (SEQ ID NO:1022). Table 86 below describes the starting and ending position of this segment on each transcript.

TABLE 86
Segment location on transcripts

| | Segment | Segment |
|---|---|---|
| Transcript name | starting position | ending position |
| T23657_T0 (SEQ ID NO: 998) | 939 | 1007 |
| T23657_T1 (SEQ ID NO: 999) | 939 | 1007 |
| T23657_T2 (SEQ ID NO: 1000) | 939 | 1007 |
| T23657_T3 (SEQ ID NO: 1001) | 939 | 1007 |
| T23657_T4 (SEQ ID NO: 1002) | 939 | 1007 |
| T23657_T5 (SEQ ID NO: 1003) | 939 | 1007 |
| T23657_T6 (SEQ ID NO: 1004) | 939 | 1007 |
| T23657_T7 (SEQ ID NO: 1005) | 939 | 1007 |
| T23657_T8 (SEQ ID NO: 1006) | 939 | 1007 |
| T23657_T9 (SEQ ID NO: 1007) | 939 | 1007 |
| T23657_T10 (SEQ ID NO: 1008) | 939 | 1007 |
| T23657_T11 (SEQ ID NO: 1009) | 939 | 1007 |
| T23657_T12 (SEQ ID NO: 1010) | 939 | 1007 |
| T23657_T13 (SEQ ID NO: 1011) | 939 | 1007 |
| T23657_T14 (SEQ ID NO: 1012) | 939 | 1007 |
| T23657_T15 (SEQ ID NO: 1013) | 939 | 1007 |
| T23657_T16 (SEQ ID NO: 1014) | 939 | 1007 |
| T23657_T17 (SEQ ID NO: 1015) | 939 | 1007 |
| T23657_T19 (SEQ ID NO: 1016) | 939 | 1007 |
| T23657_T20 (SEQ ID NO: 1017) | 939 | 1007 |
| T23657_T21 (SEQ ID NO: 1018) | 939 | 1007 |
| T23657_T22 (SEQ ID NO: 1019) | 939 | 1007 |
| T23657_T23 (SEQ ID NO: 1020) | 939 | 1007 |
| T23657_T24 (SEQ ID NO: 1021) | 939 | 1007 |
| T23657_T28 (SEQ ID NO: 1022) | 939 | 1007 |

Segment cluster T23657_node_{—}6 (SEQ ID NO:1048) according to the present invention is supported by 28 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T24 (SEQ ID NO:1021) and T23657_T28 (SEQ ID NO:1022). Table 87 below describes the starting and ending position of this segment on each transcript.

TABLE 87
Segment location on transcripts

| | Segment | Segment |
|---|---|---|
| Transcript name | starting position | ending position |
| T23657_T0 (SEQ ID NO: 998) | 1008 | 1098 |
| T23657_T1 (SEQ ID NO: 999) | 1008 | 1098 |
| T23657_T2 (SEQ ID NO: 1000) | 1008 | 1098 |
| T23657_T3 (SEQ ID NO: 1001) | 1008 | 1098 |
| T23657_T4 (SEQ ID NO: 1002) | 1008 | 1098 |
| T23657_T5 (SEQ ID NO: 1003) | 1008 | 1098 |
| T23657_T6 (SEQ ID NO: 1004) | 1008 | 1098 |
| T23657_T7 (SEQ ID NO: 1005) | 1008 | 1098 |
| T23657_T8 (SEQ ID NO: 1006) | 1008 | 1098 |
| T23657_T9 (SEQ ID NO: 1007) | 1008 | 1098 |
| T23657_T10 (SEQ ID NO: 1008) | 1008 | 1098 |
| T23657_T11 (SEQ ID NO: 1009) | 1008 | 1098 |
| T23657_T12 (SEQ ID NO: 1010) | 1008 | 1098 |
| T23657_T13 (SEQ ID NO: 1011) | 1008 | 1098 |
| T23657_T15 (SEQ ID NO: 1013) | 1008 | 1098 |
| T23657_T16 (SEQ ID NO: 1014) | 1008 | 1098 |
| T23657_T17 (SEQ ID NO: 1015) | 1008 | 1098 |
| T23657_T19 (SEQ ID NO: 1016) | 1008 | 1098 |
| T23657_T20 (SEQ ID NO: 1017) | 1008 | 1098 |
| T23657_T21 (SEQ ID NO: 1018) | 1008 | 1098 |
| T23657_T22 (SEQ ID NO: 1019) | 1008 | 1098 |
| T23657_T23 (SEQ ID NO: 1020) | 1008 | 1098 |
| T23657_T24 (SEQ ID NO: 1021) | 1008 | 1098 |
| T23657_T28 (SEQ ID NO: 1022) | 1008 | 1098 |

Segment cluster T23657_node_{—}11 (SEQ ID NO:1049) according to the present invention is supported by 33 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T24 (SEQ ID NO:1021) and T23657_T28 (SEQ ID NO:1022). Table 88 below describes the starting and ending position of this segment on each transcript.

TABLE 88
Segment location on transcripts

| | Segment | Segment |
|---|---|---|
| Transcript name | starting position | ending position |
| T23657_T0 (SEQ ID NO: 998) | 1221 | 1332 |
| T23657_T1 (SEQ ID NO: 999) | 1221 | 1332 |
| T23657_T2 (SEQ ID NO: 1000) | 1221 | 1332 |
| T23657_T3 (SEQ ID NO: 1001) | 1221 | 1332 |
| T23657_T4 (SEQ ID NO: 1002) | 1221 | 1332 |
| T23657_T5 (SEQ ID NO: 1003) | 1221 | 1332 |
| T23657_T6 (SEQ ID NO: 1004) | 1221 | 1332 |
| T23657_T7 (SEQ ID NO: 1005) | 1221 | 1332 |
| T23657_T8 (SEQ ID NO: 1006) | 1221 | 1332 |
| T23657_T9 (SEQ ID NO: 1007) | 1221 | 1332 |
| T23657_T10 (SEQ ID NO: 1008) | 1221 | 1332 |
| T23657_T11 (SEQ ID NO: 1009) | 1221 | 1332 |
| T23657_T12 (SEQ ID NO: 1010) | 1221 | 1332 |
| T23657_T13 (SEQ ID NO: 1011) | 1221 | 1332 |
| T23657_T14 (SEQ ID NO: 1012) | 1130 | 1241 |
| T23657_T15 (SEQ ID NO: 1013) | 1221 | 1332 |
| T23657_T16 (SEQ ID NO: 1014) | 1221 | 1332 |
| T23657_T17 (SEQ ID NO: 1015) | 1221 | 1332 |
| T23657_T19 (SEQ ID NO: 1016) | 1221 | 1332 |
| T23657_T20 (SEQ ID NO: 1017) | 1221 | 1332 |
| T23657_T21 (SEQ ID NO: 1018) | 1221 | 1332 |
| T23657_T22 (SEQ ID NO: 1019) | 1221 | 1332 |
| T23657_T23 (SEQ ID NO: 1020) | 1221 | 1332 |
| T23657_T24 (SEQ ID NO: 1021) | 1221 | 1332 |
| T23657_T28 (SEQ ID NO: 1022) | 1221 | 1332 |

Segment cluster T23657_node_{—}20 (SEQ ID NO:1050) according to the present invention is supported by 2 libraries. The number of libraries was determined as previously described. This segment can be found in the following transcript(s): T23657_T32 (SEQ ID NO:1025) and T23657_T37 (SEQ ID NO:1027). Table 89 below describes the starting and ending position of this segment on each transcript.

TABLE 89
Segment location on transcripts

| | Segment | Segment |
|---|---|---|
| Transcript name | starting position | ending position |
| T23657_T32 (SEQ ID NO: 1025) | 1 | 73 |
| T23657_T37 (SEQ ID NO: 1027) | 1 | 73 |

Segment cluster T23657_node_{—}22 (SEQ ID NO:1051) according to the present invention is supported by 3 libraries. The number of libraries was determined as previously described. This segment can be found in the following transcript(s): T23657_T30 (SEQ ID NO:1023), T23657_T35 (SEQ ID NO:1026) and T23657_T38 (SEQ ID NO:1028). Table 90 below describes the starting and ending position of this segment on each transcript.

TABLE 90
Segment location on transcripts

| | Segment | Segment |
|---|---|---|
| Transcript name | starting position | ending position |
| T23657_T30 (SEQ ID NO: 1023) | 1 | 117 |
| T23657_T35 (SEQ ID NO: 1026) | 1 | 117 |
| T23657_T38 (SEQ ID NO: 1028) | 1 | 117 |

Segment cluster T23657_node_{—}25 (SEQ ID NO:1052) according to the present invention is supported by 36 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T24 (SEQ ID NO:1021), T23657_T28 (SEQ ID NO:1022), T23657_T30 (SEQ ID NO:1023), T23657_T31 (SEQ ID NO:1024), T23657_T32 (SEQ ID NO:1025), T23657_T35 (SEQ ID NO:1026), T23657_T37 (SEQ ID NO:1027) and T23657_T38 (SEQ ID NO:1028). Table 91 below describes the starting and ending position of this segment on each transcript.

TABLE 91
Segment location on transcripts

| | Segment | Segment |
|---|---|---|
| Transcript name | starting position | ending position |
| T23657_T0 (SEQ ID NO: 998) | 1809 | 1849 |
| T23657_T1 (SEQ ID NO: 999) | 1809 | 1849 |
| T23657_T2 (SEQ ID NO: 1000) | 1809 | 1849 |
| T23657_T3 (SEQ ID NO: 1001) | 1809 | 1849 |
| T23657_T4 (SEQ ID NO: 1002) | 1809 | 1849 |
| T23657_T5 (SEQ ID NO: 1003) | 1809 | 1849 |
| T23657_T6 (SEQ ID NO: 1004) | 1809 | 1849 |
| T23657_T7 (SEQ ID NO: 1005) | 1809 | 1849 |
| T23657_T8 (SEQ ID NO: 1006) | 1809 | 1849 |
| T23657_T9 (SEQ ID NO: 1007) | 1809 | 1849 |
| T23657_T10 (SEQ ID NO: 1008) | 1809 | 1849 |
| T23657_T11 (SEQ ID NO: 1009) | 1809 | 1849 |
| T23657_T12 (SEQ ID NO: 1010) | 1809 | 1849 |
| T23657_T13 (SEQ ID NO: 1011) | 1809 | 1849 |
| T23657_T14 (SEQ ID NO: 1012) | 1718 | 1758 |
| T23657_T15 (SEQ ID NO: 1013) | 1809 | 1849 |
| T23657_T16 (SEQ ID NO: 1014) | 1809 | 1849 |
| T23657_T17 (SEQ ID NO: 1015) | 1809 | 1849 |
| T23657_T19 (SEQ ID NO: 1016) | 1809 | 1849 |
| T23657_T20 (SEQ ID NO: 1017) | 1809 | 1849 |
| T23657_T21 (SEQ ID NO: 1018) | 1809 | 1849 |
| T23657_T22 (SEQ ID NO: 1019) | 1809 | 1849 |
| T23657_T23 (SEQ ID NO: 1020) | 1613 | 1653 |
| T23657_T24 (SEQ ID NO: 1021) | 1809 | 1849 |
| T23657_T28 (SEQ ID NO: 1022) | 1809 | 1849 |
| T23657_T30 (SEQ ID NO: 1023) | 401 | 441 |
| T23657_T31 (SEQ ID NO: 1024) | 241 | 281 |
| T23657_T32 (SEQ ID NO: 1025) | 199 | 239 |
| T23657_T35 (SEQ ID NO: 1026) | 401 | 441 |
| T23657_T37 (SEQ ID NO: 1027) | 199 | 239 |
| T23657_T38 (SEQ ID NO: 1028) | 118 | 158 |

Segment cluster T23657_node_{—}26 (SEQ ID NO:1053) according to the present invention is supported by 8 libraries. The number of libraries was determined as previously described. This segment can be found in the following transcript(s): T23657_T10 (SEQ ID NO:1008), T23657_T11 (SEQ ID NO:1009) and T23657_T35 (SEQ ID NO:1026). Table 92 below describes the starting and ending position of this segment on each transcript.

TABLE 92
Segment location on transcripts

| | Segment | Segment |
|---|---|---|
| Transcript name | starting position | ending position |
| T23657_T10 (SEQ ID NO: 1008) | 1850 | 1950 |
| T23657_T11 (SEQ ID NO: 1009) | 1850 | 1950 |
| T23657_T35 (SEQ ID NO: 1026) | 442 | 542 |

Segment cluster T23657_node_{—}28 (SEQ ID NO:1054) according to the present invention is supported by 34 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T13 (SEQ ID NO:1011), T23657_T14 (SEQ ID NO:1012), T23657_T15 (SEQ ID NO:1013), T23657_T16 (SEQ ID NO:1014), T23657_T19 (SEQ ID NO:1016), T23657_T20 (SEQ ID NO:1017), T23657_T21 (SEQ ID NO:1018), T23657_T24 (SEQ ID NO:1021), T23657_T28 (SEQ ID NO:1022), T23657_T30 (SEQ ID NO:1023), T23657_T31 (SEQ ID NO:1024), T23657_T32 (SEQ ID NO:1025), T23657_T35 (SEQ ID NO:1026) and T23657_T38 (SEQ ID NO:1028). Table 93 below describes the starting and ending position of this segment on each transcript.

TABLE 93
Segment location on transcripts

| | Segment | Segment |
|---|---|---|
| Transcript name | starting position | ending position |
| T23657_T0 (SEQ ID NO: 998) | 1993 | 2022 |
| T23657_T1 (SEQ ID NO: 999) | 1993 | 2022 |
| T23657_T2 (SEQ ID NO: 1000) | 1993 | 2022 |
| T23657_T3 (SEQ ID NO: 1001) | 1993 | 2022 |
| T23657_T4 (SEQ ID NO: 1002) | 1993 | 2022 |
| T23657_T5 (SEQ ID NO: 1003) | 1993 | 2022 |
| T23657_T6 (SEQ ID NO: 1004) | 1993 | 2022 |
| T23657_T7 (SEQ ID NO: 1005) | 1993 | 2022 |
| T23657_T8 (SEQ ID NO: 1006) | 1993 | 2022 |
| T23657_T9 (SEQ ID NO: 1007) | 1993 | 2022 |
| T23657_T10 (SEQ ID NO: 1008) | 2094 | 2123 |
| T23657_T11 (SEQ ID NO: 1009) | 2094 | 2123 |
| T23657_T13 (SEQ ID NO: 1011) | 1850 | 1879 |
| T23657_T14 (SEQ ID NO: 1012) | 1902 | 1931 |
| T23657_T15 (SEQ ID NO: 1013) | 1993 | 2022 |
| T23657_T16 (SEQ ID NO: 1014) | 1993 | 2022 |
| T23657_T19 (SEQ ID NO: 1016) | 1850 | 1879 |
| T23657_T20 (SEQ ID NO: 1017) | 1993 | 2022 |
| T23657_T21 (SEQ ID NO: 1018) | 1993 | 2022 |
| T23657_T24 (SEQ ID NO: 1021) | 1993 | 2022 |
| T23657_T28 (SEQ ID NO: 1022) | 1850 | 1879 |
| T23657_T30 (SEQ ID NO: 1023) | 585 | 614 |
| T23657_T31 (SEQ ID NO: 1024) | 425 | 454 |
| T23657_T32 (SEQ ID NO: 1025) | 383 | 412 |
| T23657_T35 (SEQ ID NO: 1026) | 686 | 715 |
| T23657_T38 (SEQ ID NO: 1028) | 159 | 188 |

Segment cluster T23657_node_{—}30 (SEQ ID NO:1055) according to the present invention is supported by 7 libraries. The number of libraries was determined as previously described. This segment can be found in the following transcript(s): T23657_T5 (SEQ ID NO:1003), T23657_T6 (SEQ ID NO:1004), T23657_T10 (SEQ ID NO:1008), T23657_T11 (SEQ ID NO:1009) and T23657_T35 (SEQ ID NO:1026). Table 94 below describes the starting and ending position of this segment on each transcript.

TABLE 94
Segment location on transcripts

| | Segment | Segment |
|---|---|---|
| Transcript name | starting position | ending position |
| T23657_T5 (SEQ ID NO: 1003) | 2279 | 2314 |
| T23657_T6 (SEQ ID NO: 1004) | 2279 | 2314 |
| T23657_T10 (SEQ ID NO: 1008) | 2380 | 2415 |
| T23657_T11 (SEQ ID NO: 1009) | 2380 | 2415 |
| T23657_T35 (SEQ ID NO: 1026) | 972 | 1007 |

Segment cluster T23657_node_{—}31 (SEQ ID NO:1056) according to the present invention is supported by 46 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T24 (SEQ ID NO:1021), T23657_T28 (SEQ ID NO:1022), T23657_T30 (SEQ ID NO:1023), T23657_T31 (SEQ ID NO:1024), T23657_T32 (SEQ ID NO:1025), T23657_T35 (SEQ ID NO:1026), T23657_T37 (SEQ ID NO:1027) and T23657_T38 (SEQ ID NO:1028). Table 95 below describes the starting and ending position of this segment on each transcript.

TABLE 95
Segment location on transcripts

| | Segment | Segment |
|---|---|---|
| Transcript name | starting position | ending position |
| T23657_T0 (SEQ ID NO: 998) | 2023 | 2087 |
| T23657_T1 (SEQ ID NO: 999) | 2023 | 2087 |
| T23657_T2 (SEQ ID NO: 1000) | 2023 | 2087 |
| T23657_T3 (SEQ ID NO: 1001) | 2023 | 2087 |
| T23657_T4 (SEQ ID NO: 1002) | 2023 | 2087 |
| T23657_T5 (SEQ ID NO: 1003) | 2315 | 2379 |
| T23657_T6 (SEQ ID NO: 1004) | 2315 | 2379 |
| T23657_T7 (SEQ ID NO: 1005) | 2023 | 2087 |
| T23657_T8 (SEQ ID NO: 1006) | 2023 | 2087 |
| T23657_T9 (SEQ ID NO: 1007) | 2023 | 2087 |
| T23657_T10 (SEQ ID NO: 1008) | 2416 | 2480 |
| T23657_T11 (SEQ ID NO: 1009) | 2416 | 2480 |
| T23657_T12 (SEQ ID NO: 1010) | 1850 | 1914 |
| T23657_T13 (SEQ ID NO: 1011) | 1880 | 1944 |
| T23657_T14 (SEQ ID NO: 1012) | 1932 | 1996 |
| T23657_T15 (SEQ ID NO: 1013) | 2023 | 2087 |
| T23657_T16 (SEQ ID NO: 1014) | 2023 | 2087 |
| T23657_T17 (SEQ ID NO: 1015) | 1850 | 1914 |
| T23657_T19 (SEQ ID NO: 1016) | 1880 | 1944 |
| T23657_T20 (SEQ ID NO: 1017) | 2023 | 2087 |
| T23657_T21 (SEQ ID NO: 1018) | 2023 | 2087 |
| T23657_T22 (SEQ ID NO: 1019) | 1850 | 1914 |
| T23657_T23 (SEQ ID NO: 1020) | 1654 | 1718 |
| T23657_T24 (SEQ ID NO: 1021) | 2023 | 2087 |
| T23657_T28 (SEQ ID NO: 1022) | 1880 | 1944 |
| T23657_T30 (SEQ ID NO: 1023) | 615 | 679 |
| T23657_T31 (SEQ ID NO: 1024) | 455 | 519 |
| T23657_T32 (SEQ ID NO: 1025) | 413 | 477 |
| T23657_T35 (SEQ ID NO: 1026) | 1008 | 1072 |
| T23657_T37 (SEQ ID NO: 1027) | 240 | 304 |
| T23657_T38 (SEQ ID NO: 1028) | 189 | 253 |

Segment cluster T23657_node_{—}32 (SEQ ID NO:1057) according to the present invention is supported by 5 libraries. The number of libraries was determined as previously described. This segment can be found in the following transcript(s): T23657_T4 (SEQ ID NO:1002), T23657_T6 (SEQ ID NO:1004) and T23657_T15 (SEQ ID NO:1013). Table 96 below describes the starting and ending position of this segment on each transcript.

TABLE 96
Segment location on transcripts

| | Segment | Segment |
|---|---|---|
| Transcript name | starting position | ending position |
| T23657_T4 (SEQ ID NO: 1002) | 2088 | 2150 |
| T23657_T6 (SEQ ID NO: 1004) | 2380 | 2442 |
| T23657_T15 (SEQ ID NO: 1013) | 2088 | 2150 |

Segment cluster T23657_node_{—}41 (SEQ ID NO:1058) according to the present invention is supported by 26 libraries. The number of libraries was determined as previously described. This segment can be found in the following transcript(s): T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T9 (SEQ ID NO:1007), T23657_T10 (SEQ ID NO:1008), T23657_T12 (SEQ ID NO:1010), T23657_T13 (SEQ ID NO:1011), T23657_T16 (SEQ ID NO:1014) and T23657_T35 (SEQ ID NO:1026). Table 97 below describes the starting and ending position of this segment on each transcript.

TABLE 97
Segment location on transcripts

| | Segment | Segment |
|---|---|---|
| Transcript name | starting position | ending position |
| T23657_T2 (SEQ ID NO: 1000) | 3974 | 4033 |
| T23657_T3 (SEQ ID NO: 1001) | 4272 | 4331 |
| T23657_T4 (SEQ ID NO: 1002) | 4335 | 4394 |
| T23657_T5 (SEQ ID NO: 1003) | 4266 | 4325 |
| T23657_T6 (SEQ ID NO: 1004) | 4627 | 4686 |
| T23657_T7 (SEQ ID NO: 1005) | 3974 | 4033 |
| T23657_T9 (SEQ ID NO: 1007) | 4272 | 4331 |
| T23657_T10 (SEQ ID NO: 1008) | 4367 | 4426 |
| T23657_T12 (SEQ ID NO: 1010) | 3801 | 3860 |
| T23657_T13 (SEQ ID NO: 1011) | 4129 | 4188 |
| T23657_T16 (SEQ ID NO: 1014) | 3974 | 4033 |
| T23657_T35 (SEQ ID NO: 1026) | 2959 | 3018 |

Segment cluster T23657_node_{—}42 (SEQ ID NO:1059) according to the present invention is supported by 71 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T30 (SEQ ID NO:1023), T23657_T31 (SEQ ID NO:1024), T23657_T32 (SEQ ID NO:1025), T23657_T35 (SEQ ID NO:1026) and T23657_T37 (SEQ ID NO:1027). Table 98 below describes the starting and ending position of this segment on each transcript.

TABLE 98
Segment location on transcripts

| | Segment | Segment |
|---|---|---|
| Transcript name | starting position | ending position |
| T23657_T0 (SEQ ID NO: 998) | 2237 | 2268 |
| T23657_T1 (SEQ ID NO: 999) | 2237 | 2268 |
| T23657_T2 (SEQ ID NO: 1000) | 4034 | 4065 |
| T23657_T3 (SEQ ID NO: 1001) | 4332 | 4363 |
| T23657_T4 (SEQ ID NO: 1002) | 4395 | 4426 |
| T23657_T5 (SEQ ID NO: 1003) | 4326 | 4357 |
| T23657_T6 (SEQ ID NO: 1004) | 4687 | 4718 |
| T23657_T7 (SEQ ID NO: 1005) | 4034 | 4065 |
| T23657_T8 (SEQ ID NO: 1006) | 2237 | 2268 |
| T23657_T9 (SEQ ID NO: 1007) | 4332 | 4363 |
| T23657_T10 (SEQ ID NO: 1008) | 4427 | 4458 |
| T23657_T11 (SEQ ID NO: 1009) | 2630 | 2661 |
| T23657_T12 (SEQ ID NO: 1010) | 3861 | 3892 |
| T23657_T13 (SEQ ID NO: 1011) | 4189 | 4220 |
| T23657_T14 (SEQ ID NO: 1012) | 2146 | 2177 |
| T23657_T15 (SEQ ID NO: 1013) | 2300 | 2331 |
| T23657_T17 (SEQ ID NO: 1015) | 2064 | 2095 |
| T23657_T19 (SEQ ID NO: 1016) | 2094 | 2125 |
| T23657_T20 (SEQ ID NO: 1017) | 2407 | 2438 |
| T23657_T21 (SEQ ID NO: 1018) | 2377 | 2408 |
| T23657_T22 (SEQ ID NO: 1019) | 2234 | 2265 |
| T23657_T23 (SEQ ID NO: 1020) | 1868 | 1899 |
| T23657_T30 (SEQ ID NO: 1023) | 829 | 860 |
| T23657_T31 (SEQ ID NO: 1024) | 669 | 700 |
| T23657_T32 (SEQ ID NO: 1025) | 627 | 658 |
| T23657_T35 (SEQ ID NO: 1026) | 3019 | 3050 |
| T23657_T37 (SEQ ID NO: 1027) | 454 | 485 |

Segment cluster T23657_node_{—}43 (SEQ ID NO:1060) according to the present invention is supported by 80 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T30 (SEQ ID NO:1023), T23657_T31 (SEQ ID NO:1024), T23657_T32 (SEQ ID NO:1025), T23657_T35 (SEQ ID NO:1026) and T23657_T37 (SEQ ID NO:1027). Table 99 below describes the starting and ending position of this segment on each transcript.

TABLE 99
Segment location on transcripts

| | Segment | Segment |
|---|---|---|
| Transcript name | starting position | ending position |
| T23657_T0 (SEQ ID NO: 998) | 2269 | 2329 |
| T23657_T1 (SEQ ID NO: 999) | 2269 | 2329 |
| T23657_T2 (SEQ ID NO: 1000) | 4066 | 4126 |
| T23657_T3 (SEQ ID NO: 1001) | 4364 | 4424 |
| T23657_T4 (SEQ ID NO: 1002) | 4427 | 4487 |
| T23657_T5 (SEQ ID NO: 1003) | 4358 | 4418 |
| T23657_T6 (SEQ ID NO: 1004) | 4719 | 4779 |
| T23657_T7 (SEQ ID NO: 1005) | 4066 | 4126 |
| T23657_T8 (SEQ ID NO: 1006) | 2269 | 2329 |
| T23657_T9 (SEQ ID NO: 1007) | 4364 | 4424 |
| T23657_T10 (SEQ ID NO: 1008) | 4459 | 4519 |
| T23657_T11 (SEQ ID NO: 1009) | 2662 | 2722 |
| T23657_T12 (SEQ ID NO: 1010) | 3893 | 3953 |
| T23657_T13 (SEQ ID NO: 1011) | 4221 | 4281 |
| T23657_T14 (SEQ ID NO: 1012) | 2178 | 2238 |
| T23657_T15 (SEQ ID NO: 1013) | 2332 | 2392 |
| T23657_T17 (SEQ ID NO: 1015) | 2096 | 2156 |
| T23657_T19 (SEQ ID NO: 1016) | 2126 | 2186 |
| T23657_T20 (SEQ ID NO: 1017) | 2439 | 2499 |
| T23657_T21 (SEQ ID NO: 1018) | 2409 | 2469 |
| T23657_T22 (SEQ ID NO: 1019) | 2266 | 2326 |
| T23657_T23 (SEQ ID NO: 1020) | 1900 | 1960 |
| T23657_T30 (SEQ ID NO: 1023) | 861 | 921 |
| T23657_T31 (SEQ ID NO: 1024) | 701 | 761 |
| T23657_T32 (SEQ ID NO: 1025) | 659 | 719 |
| T23657_T35 (SEQ ID NO: 1026) | 3051 | 3111 |
| T23657_T37 (SEQ ID NO: 1027) | 486 | 546 |

Segment cluster T23657_node_{—}44 (SEQ ID NO:1061) according to the present invention is supported by 79 libraries. The number of libraries was determined as previously described. This segment can be found in the following transcript(s): T23657_T0 (SEQ ID NO:998), T23657_T1 (SEQ ID NO:999), T23657_T2 (SEQ ID NO:1000), T23657_T3 (SEQ ID NO:1001), T23657_T4 (SEQ ID NO:1002), T23657_T5 (SEQ ID NO:1003), T23657_T6 (SEQ ID NO:1004), T23657_T7 (SEQ ID NO:1005), T23657_T8 (SEQ ID NO:1006), T23657_T9 (SEQ ID NO:1007), T23657_T10 (SEQ ID NO:1008), T23657_T11 (SEQ ID NO:1009), T23657_T12 (SEQ ID NO:1010), T23657_T13 (SEQ ID NO:1011), T23657_T14 (SEQ ID NO:1012), T23657_T15 (SEQ ID NO:1013), T23657_T16 (SEQ ID NO:1014), T23657_T17 (SEQ ID NO:1015), T23657_T19 (SEQ ID NO:1016), T23657_T20 (SEQ ID NO:1017), T23657_T21 (SEQ ID NO:1018), T23657_T22 (SEQ ID NO:1019), T23657_T23 (SEQ ID NO:1020), T23657_T30 (SEQ ID NO:1023), T23657_T31 (SEQ ID NO:1024), T23657_T32 (SEQ ID NO:1025), T23657_T35 (SEQ ID NO:1026) and T23657_T37 (SEQ ID NO:1027). Table 100 below describes the starting and ending position of this segment on each transcript.

TABLE 100
Segment location on transcripts

| | Segment | Segment |
|---|---|---|
| Transcript name | starting position | ending position |
| T23657_T0 (SEQ ID NO: 998) | 2330 | 2362 |
| T23657_T1 (SEQ ID NO: 999) | 2330 | 2362 |
| T23657_T2 (SEQ ID NO: 1000) | 4127 | 4159 |
| T23657_T3 (SEQ ID NO: 1001) | 4425 | 4457 |
| T23657_T4 (SEQ ID NO: 1002) | 4488 | 4520 |
| T23657_T5 (SEQ ID NO: 1003) | 4419 | 4451 |
| T23657_T6 (SEQ ID NO: 1004) | 4780 | 4812 |
| T23657_T7 (SEQ ID NO: 1005) | 4127 | 4159 |
| T23657_T8 (SEQ ID NO: 1006) | 2330 | 2362 |
| T23657_T9 (SEQ ID NO: 1007) | 4425 | 4457 |
| T23657_T10 (SEQ ID NO: 1008) | 4520 | 4552 |
| T23657_T11 (SEQ ID NO: 1009) | 2723 | 2755 |
| T23657_T12 (SEQ ID NO: 1010) | 3954 | 3986 |
| T23657_T13 (SEQ ID NO: 1011) | 4282 | 4314 |
| T23657_T14 (SEQ ID NO: 1012) | 2239 | 2271 |
| T23657_T15 (SEQ ID NO: 1013) | 2393 | 2425 |
| T23657_T16 (SEQ ID NO: 1014) | 4034 | 4066 |
| T23657_T17 (SEQ ID NO: 1015) | 2157 | 2189 |
| T23657_T19 (SEQ ID NO: 1016) | 2187 | 2219 |
| T23657_T20 (SEQ ID NO: 1017) | 2500 | 2532 |
| T23657_T21 (SEQ ID NO: 1018) | 2470 | 2502 |
| T23657_T22 (SEQ ID NO: 1019) | 2327 | 2359 |
| T23657_T23 (SEQ ID NO: 1020) | 1961 | 1993 |
| T23657_T30 (SEQ ID NO: 1023) | 922 | 954 |
| T23657_T31 (SEQ ID NO: 1024) | 762 | 794 |
| T23657_T32 (SEQ ID NO: 1025) | 720 | 752 |
| T23657_T35 (SEQ ID NO: 1026) | 3112 | 3144 |
| T23657_T37 (SEQ ID NO: 1027) | 547 | 579 |

### Variant Protein Alignment to the Previously Known Protein:

Sequence name: S21C_HUMAN (SEQ ID NO:1062)
Sequence documentation:
Alignment of: T23657_P2 (SEQ ID NO:1064) × S21C_HUMAN (SEQ ID NO:1062) ..
Alignment segment 1/1:

| | | | |
|---|---|---|---|
| Quality: | 6620.00 | Escore: | 0 |
| Matching length: | 675 | Total length: | 675 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

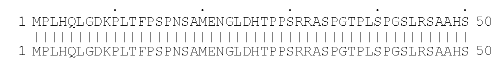

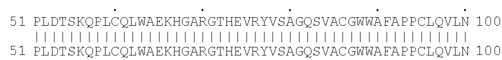

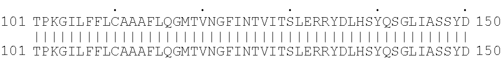

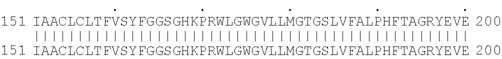

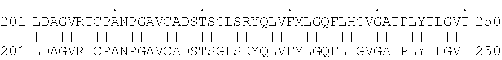

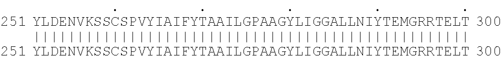

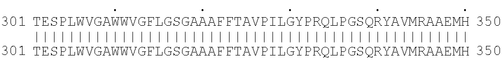

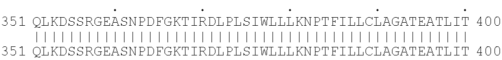

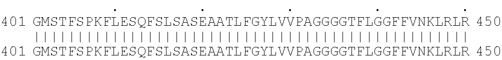

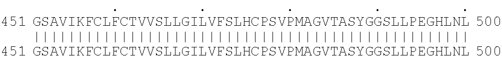

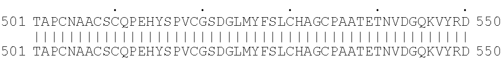

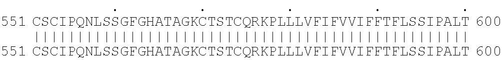

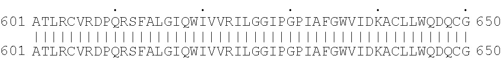

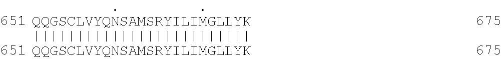

Sequence name: S21C_HUMAN (SEQ ID NO:1062)
Sequence documentation:
Alignment of: T23657_P3 (SEQ ID NO:1065) × S21C_HUMAN (SEQ ID NO:1062) ..
Alignment segment 1/1:

| | | | |
|---|---|---|---|
| Quality: | 6621.00 | Escore: | 0 |
| Matching length: | 677 | Total length: | 677 |
| Matching Percent Similarity: | 99.85 | Matching Percent Identity: | 99.70 |
| Total Percent Similarity: | 99.85 | Total Percent Identity: | 99.70 |
| Gaps: | 0 | | |
| Alignment: | | | |

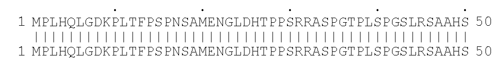

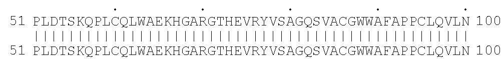

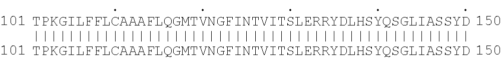

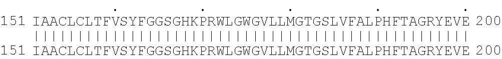

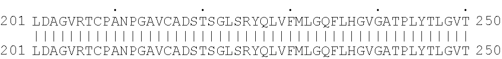

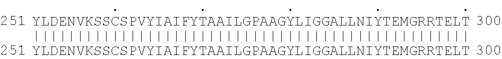

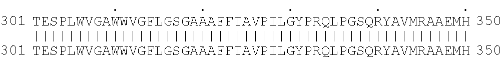

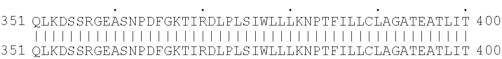

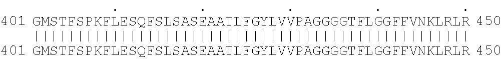

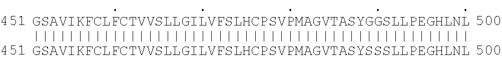

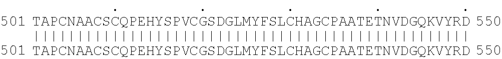

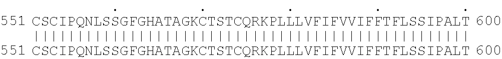

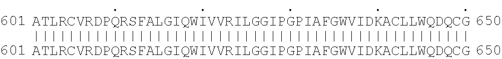

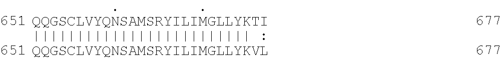

Sequence name: S21C_HUMAN (SEQ ID NO:1062)
Sequence documentation:
Alignment of: T23657_P4 (SEQ ID NO:1066) × S21C_HUMAN (SEQ ID NO:1062) ..
Alignment segment 1/1:

| | | | |
|---|---|---|---|
| Quality: | 6521.00 | Escore: | 0 |
| Matching length: | 677 | Total length: | 698 |
| Matching Percent Similarity: | 99.85 | Matching Percent Identity: | 99.70 |
| Total Percent Similarity: | 96.85 | Total Percent Identity: | 96.70 |
| Gaps: | 1 | | |
| Alignment: | | | |

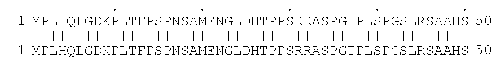

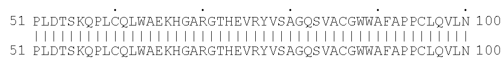

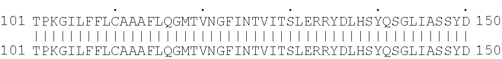

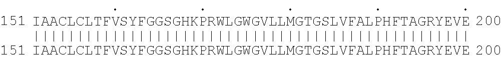

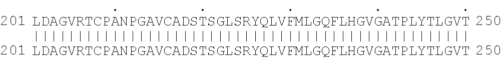

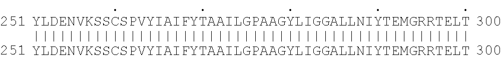

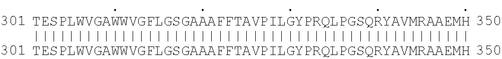

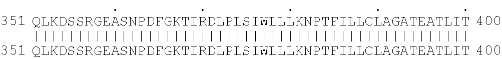

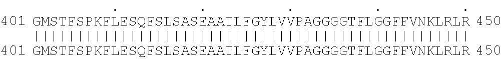

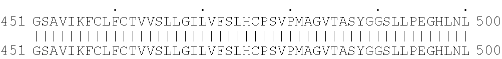

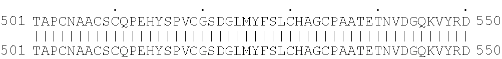

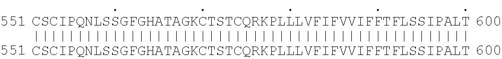

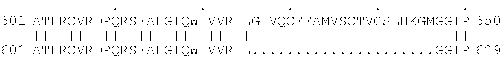

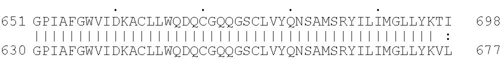

Sequence name: S21C_HUMAN (SEQ ID NO:1062)
Sequence documentation:
Alignment of: T23657_P5 (SEQ ID NO:1067) × S21C_HUMAN (SEQ ID NO:1062) ..
Alignment segment 1/1:

| | | | |
|---|---|---|---|
| Quality: | 5909.00 | Escore: | 0 |
| Matching length: | 604 | Total length: | 604 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

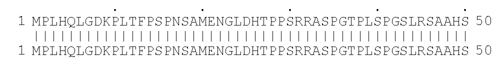

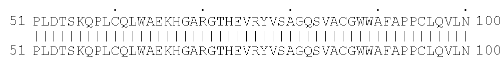

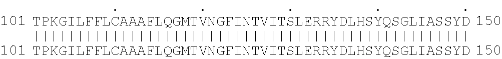

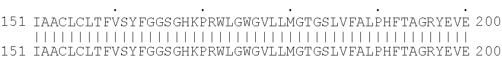

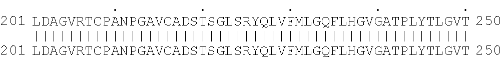

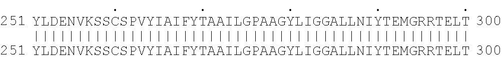

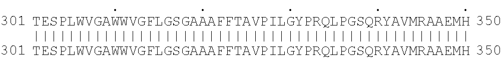

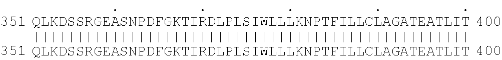

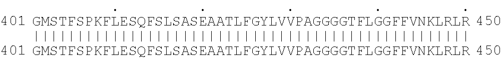

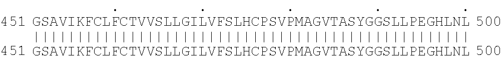

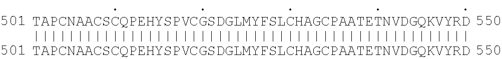

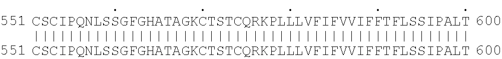

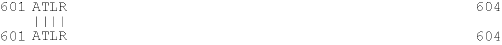

Sequence name: S21C_HUMAN (SEQ ID NO:1062)
Sequence documentation:
Alignment of: T23657_P6 (SEQ ID NO:1068) × S21C_HUMAN (SEQ ID NO:1062) ..
Alignment segment 1/1:

| | | | |
|---|---|---|---|
| Quality: | 5354.00 | Escore: | 0 |
| Matching length: | 547 | Total length: | 547 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

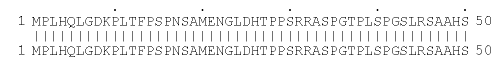

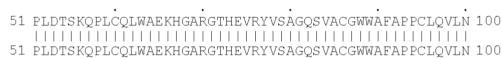

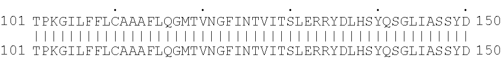

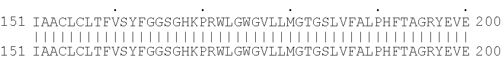

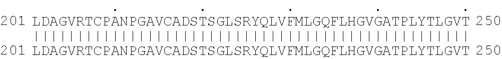

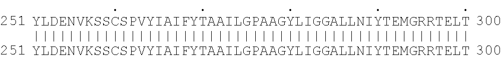

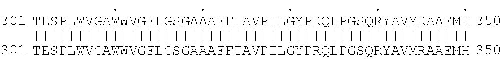

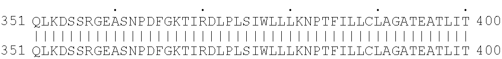

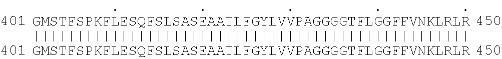

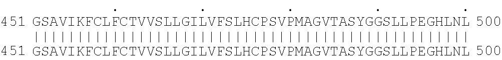

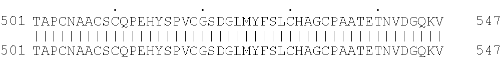

Sequence name: S21C_HUMAN (SEQ ID NO:1062)
Sequence documentation:
Alignment of: T23657_P7 (SEQ ID NO:1069) × S21C_HUMAN (SEQ ID NO:1062) ..
Alignment segment 1/1:

| | | | |
|---|---|---|---|
| Quality: | 5346.00 | Escore: | 0 |
| Matching length: | 546 | Total length: | 546 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

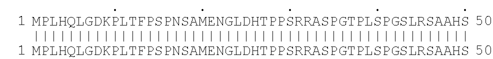

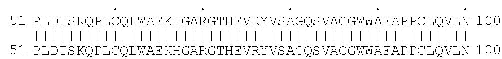

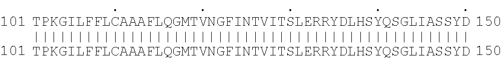

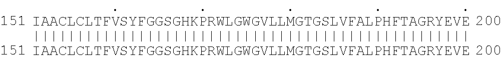

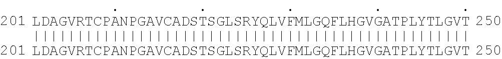

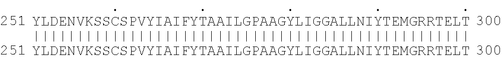

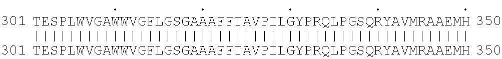

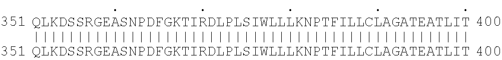

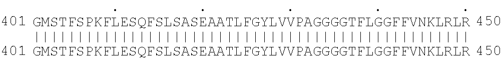

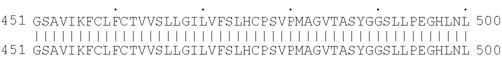

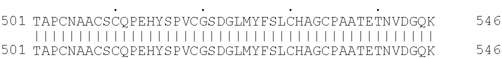

Sequence name: S21C_HUMAN (SEQ ID NO:1062)
Sequence documentation:
Alignment of: T23657_P8 (SEQ ID NO:1070) × S21C_HUMAN (SEQ ID NO:1062) ..
Alignment segment 1/1:

| | | | |
|---|---|---|---|
| Quality: | 5346.00 | Escore: | 0 |
| Matching length: | 546 | Total length: | 546 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

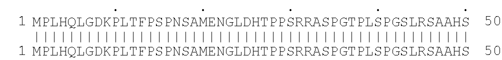

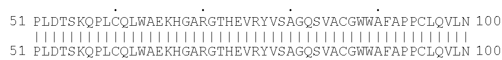

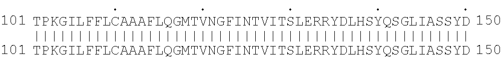

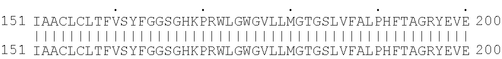

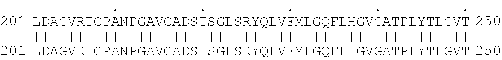

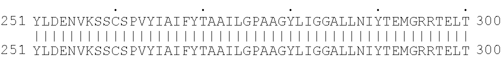

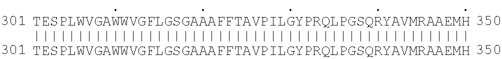

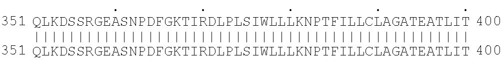

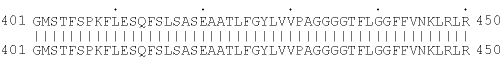

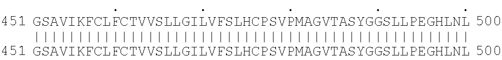

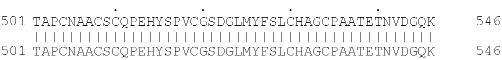

Sequence name: S21C_HUMAN (SEQ ID NO:1062)
Sequence documentation:
Alignment of: T23657_P10 (SEQ ID NO:1072) × S21C_HUMAN (SEQ ID NO:1062)
Alignment segment 1/1:

| | | | |
|---|---|---|---|
| Quality: | 6968.00 | Escore: | 0 |
| Matching length: | 722 | Total length: | 743 |
| Matching Percent Similarity: | 100.00 | Matching Percent Identity: | 100.00 |
| Total Percent Similarity: | 97.17 | Total Percent Identity: | 97.17 |
| Gaps: | 1 | | |
| Alignment: | | | |

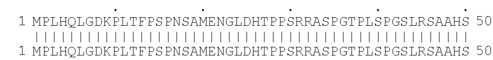

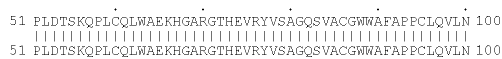

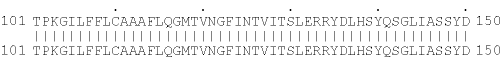

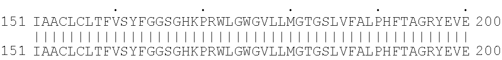

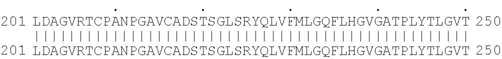

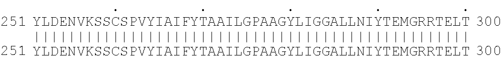

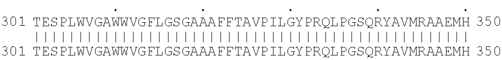

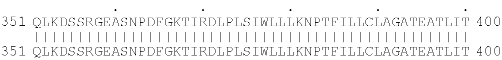

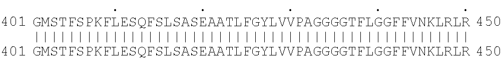

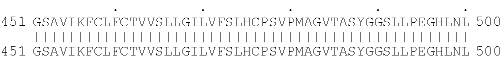

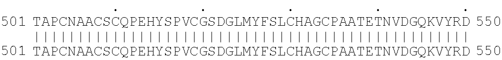

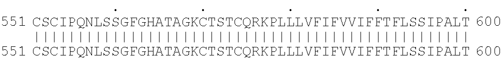

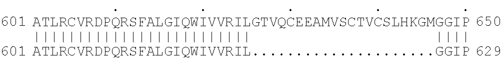

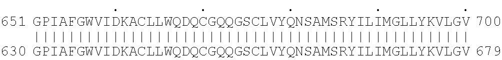

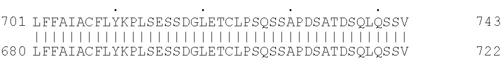

Sequence name: S21C_HUMAN (SEQ ID NO:1062)
Sequence documentation:
Alignment of: T23657_P11 (SEQ ID NO:1073) × S21C_HUMAN (SEQ ID NO:1062)
Alignment segment 1/1:

| | | | |
|---|---|---|---|
| Quality: | 4156.00 | Escore: | 0 |
| Matching length: | 425 | Total length: | 425 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

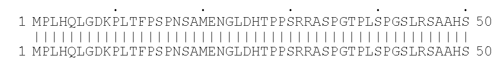

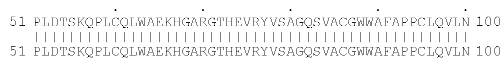

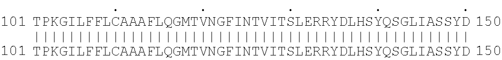

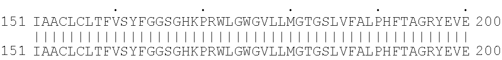

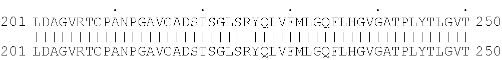

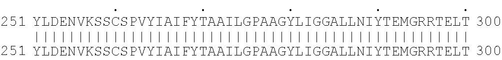

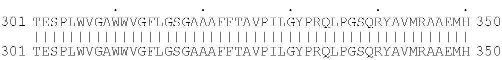

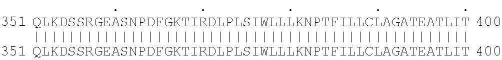

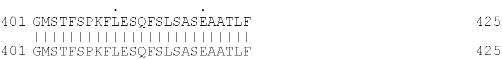

Sequence name: S21C_HUMAN (SEQ ID NO:1062)
Sequence documentation:
Alignment of: T23657_P12 (SEQ ID NO:1074) × S21C_HUMAN (SEQ ID NO:1062)
Alignment segment 1/1:

| | | | |
|---|---|---|---|
| Quality: | 6620.00 | Escore: | 0 |
| Matching length: | 675 | Total length: | 675 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

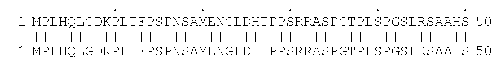

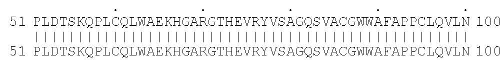

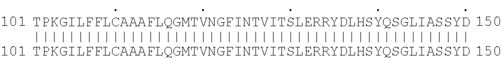

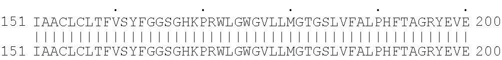

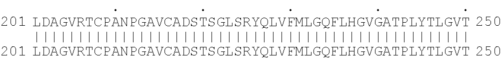

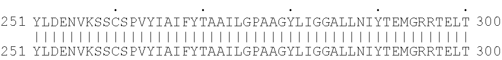

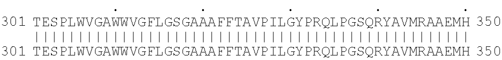

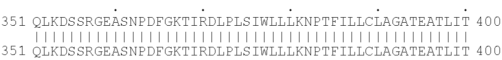

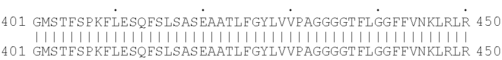

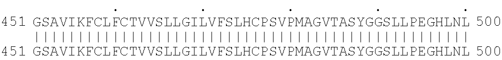

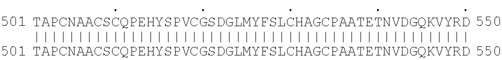

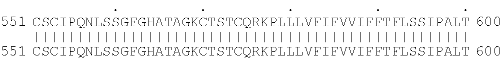

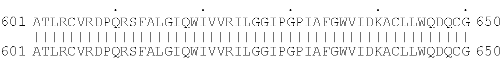

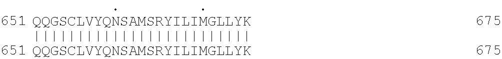

Sequence name: S2lC_HUMAN (SEQ ID NO:1062)
Sequence documentation:
Alignment of: T23657_P16 (SEQ ID NO:1075) × S21C_HUMAN (SEQ ID NO:1062)
Alignment segment 1/1:

| | | | |
|---|---|---|---|
| Quality: | 2296.00 | Escore: | 0 |
| Matching length: | 232 | Total length: | 232 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

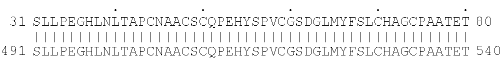

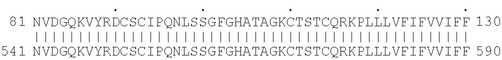

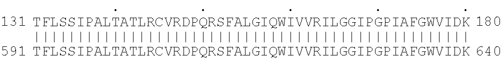

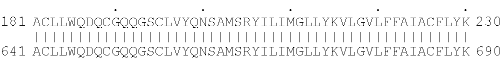

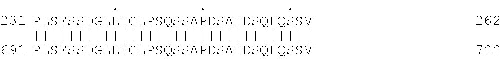

Sequence name: S21C_HUMAN (SEQ ID NO:1062)
Sequence documentation:
Alignment of: T23657_P17 (SEQ ID NO:1076) × S21C_HUMAN (SEQ ID NO:1062)
Alignment segment 1/1:

| | | | |
|---|---|---|---|
| Quality: | 1947.00 | Escore: | 0 |
| Matching length: | 198 | Total length: | 198 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

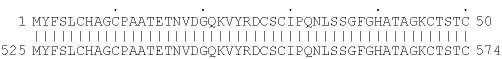

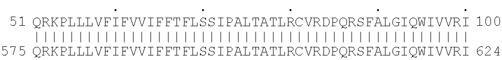

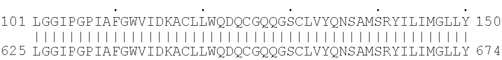

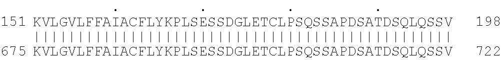

Sequence name: S21C_HUMAN (SEQ ID NO:1062)
Sequence documentation:
Alignment of: T23657_P21 (SEQ ID NO:1078) × S21C_HUMAN (SEQ ID NO:1062)
Alignment segment 1/1:

| | | | |
|---|---|---|---|
| Quality: | 1169.00 | Escore: | 0 |
| Matching length: | 119 | Total length: | 119 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

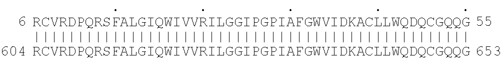

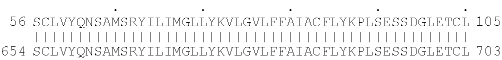

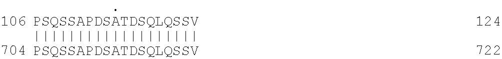

Sequence name: S21C_HUMAN (SEQ ID NO:1062)
Sequence documentation:
Alignment of: T23657_P23 (SEQ ID NO:1080) × S21C_HUMAN (SEQ ID NO:1062)
Alignment segment 1/1:

| | | | |
|---|---|---|---|
| Quality: | 5354.00 | Escore: | 0 |
| Matching length: | 547 | Total length: | 547 |
| Matching Percent Similarity: | 100.00 | Matching Percent Identity: | 100.00 |
| Total Percent Similarity: | 100.00 | Total Percent Identity: | 100.00 |
| Gaps: | 0 | | |
| Alignment: | | | |

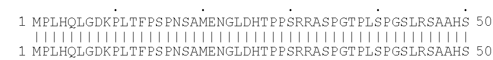

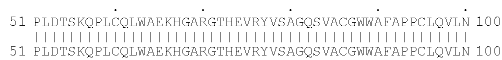

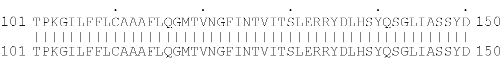

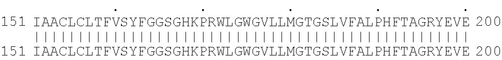

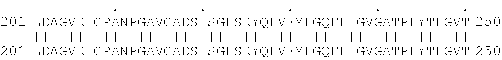

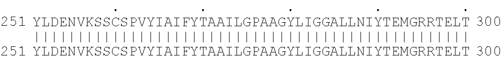

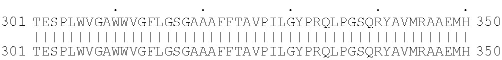

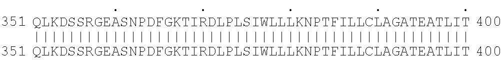

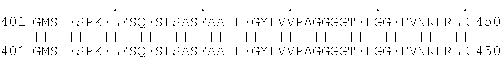

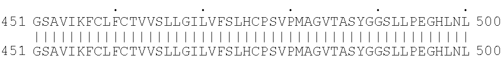

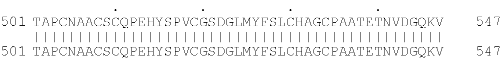

### Expression of Solute Carrier Organic Anion Transporter Family, Member 4A1 (SLCO4A1) T23657 Transcripts, which are Detectable by Amplicon as Depicted in Sequence Name T23657 Seg17-18 (SEQ ID NO: 1357), in Normal and Cancerous Colon Tissues

Expression of solute carrier organic anion transporter family, member 4A1 (SLCO4A1) transcripts detectable by or according to seg17-18, T23657 amplicon (SEQ ID NO: 1357) and T23657 Seg17-18F (SEQ ID NO: 1355) T23657 Seg17-18 R (SEQ ID NO: 1356) primers was measured by real time PCR. In parallel the expression of four housekeeping genes —PBGD (GenBank Accession No. BC019323 (SEQ ID NO:1576); amplicon —PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

FIG. 59 is a histogram showing over expression of the above-indicated solute carrier organic anion transporter family, member 4A1 (SLCO4A1) transcripts in cancerous colon samples relative to the normal samples. (Values represent the average of duplicate experiments. Error bars indicate the minimal and maximal values obtained). The number and percentage of samples that exhibit at least 4 fold over-expression, out of the total number of samples tested is indicated in the bottom.

As is evident from FIG. 59, the expression of solute carrier organic anion transporter family, member 4A1 (SLCO4A1) transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 4 fold was found in 28 out of 37 adenocarcinoma samples,

Statistical analysis was applied to verify the significance of these results, as described below.

The P value for the difference in the expression levels of solute carrier organic anion transporter family, member 4A1 (SLCO4A1) transcripts detectable by the above amplicon in colon cancer samples versus the normal tissue samples was determined by T test as 7.22E-04.

Threshold of 4 fold overexpression was found to differentiate between cancer and normal samples with P value of 7.43E-06 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: T23657seg17-18F forward primer (SEQ ID NO: 1355); and T23657seg17-18R reverse primer (SEQ ID NO: 1356).

The present invention also preferably encompasses any amplicon obtained through the use of any suitable primer pair; for example, for the above experiment, the following amplicon was obtained as a non-limiting illustrative example only of a suitable amplicon: T23657seg17-18 (SEQ ID NO: 1357).

Forward primer (SEQ ID NO: 1355):
CTGCTGGGCATCCTCGTCT
Reverse primer (SEQ ID NO: 1356):
CGTACCCAGGTGCCATCTG
Amplicon (SEQ ID NO: 1357):
CTGGGCATCCTCGTCTTCTCACTGCACTGCCCCAGTGTGCCCATGGCGGG
CGTCACAGCCAGCTACGGCGGGAGGTGAGGGCCAGATGGCACCTGGGTAC
G

### Expression of Solute Carrier Organic Anion Transporter Family, Member 4A1 (SLC04A1) T23657 Transcripts which are Detectable by Amplicon as Depicted in Sequence Name T23657 Seg22 (SEQ ID NO: 1360) in Normal and Cancerous Colon Tissues

Expression of solute carrier organic anion transporter family, member 4A1 (SLCO4A1) transcripts detectable by or according to seg22, T23657 amplicon (SEQ ID NO: 1360) and T23657 seg22F (SEQ ID NO: 1358) T23657 seg22 R (SEQ ID NO: 1359) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon —HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

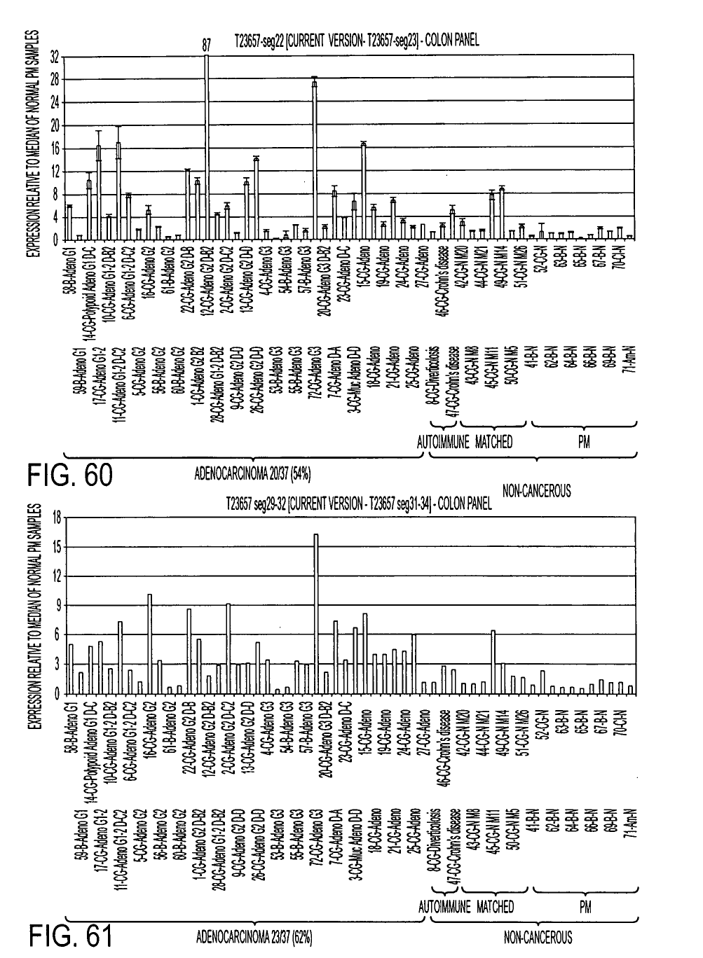

FIG. 60 is a histogram showing over expression of the above-indicated solute carrier organic anion transporter family, member 4A1 (SLCO4A1) transcripts in cancerous colon samples relative to the normal samples (values represent the average of duplicate experiments. Error bars indicate the minimal and maximal values obtained). The number and percentage of samples that exhibit at least 4 fold over-expression, out of the total number of samples tested is indicated in the bottom.

As is evident from FIG. 60, the expression of solute carrier organic anion transporter family, member 4A1 (SLCO4A1) transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 4 fold was found in 20 out of 37 adenocarcinoma samples,

Statistical analysis was applied to verify the significance of these results, as described below.

The P value for the difference in the expression levels of solute carrier organic anion transporter family, member 4A1 (SLCO4A1) transcripts detectable by the above amplicon in colon cancer samples versus the normal tissue samples was determined by T test as 3.62E-03.

Threshold of 4 fold overexpression was found to differentiate between cancer and normal samples with P value of 9.50E-04 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: T23657seg22F forward primer (SEQ ID NO: 1358); and T23657seg22R reverse primer (SEQ ID NO: 1359).

The present invention also preferably encompasses any amplicon obtained through the use of any suitable primer pair; for example, for the above experiment, the following amplicon was obtained as a non-limiting illustrative example only of a suitable amplicon: T23657seg22 (SEQ ID NO: 1360).

Forward primer (SEQ ID NO: 1358):
TGGCAAGTTTGTAGACCCGAA
Reverse primer (SEQ ID NO: 1359):
GGTAGGGTCCAGGCCAGAG
Amplicon (SEQ ID NO: 1360):
TGGCAAGTTTGTAGACCCGAAATGCAGGCTGCATGGGGACGAGCCCCATG
GCTGACCCTGTGCCTGCTGGGCGCCAGCATGGCTCTGGCCTGGACCCTAC
C

### Expression of Solute Carrier Organic Anion Transporter Family, Member 4A1 (SLC04A1) T23657 Transcripts which are Detectable by Amplicon as Depicted in Sequence Name T23657 Seg29-32 (SEQ ID NO: 1363) in Normal and Cancerous Colon Tissues

Expression of solute carrier organic anion transporter family, member 4A1 (SLCO4A1) transcripts detectable by or according to seg29-32, T23657 amplicon (SEQ ID NO: 1363) and T23657 seg29-32F (SEQ ID NO: 1361) T23657 seg29-32 R (SEQ ID NO: 1362) primers was measured by real time PCR. In parallel the expression of four housekeeping genes —PBGD (GenBank Accession No. BC019323 (SEQ ID NO:1576); amplicon —PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

FIG. 61 is a histogram showing over expression of the above-indicated solute carrier organic anion transporter family, member 4A1 (SLCO4A1) transcripts in cancerous colon samples relative to the normal samples. The number and percentage of samples that exhibit at least 3 fold over-expression, out of the total number of samples tested is indicated in the bottom.

As is evident from FIG. 61, the expression of solute carrier organic anion transporter family, member 4A1 (SLCO4A1) transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 3 fold was found in 23 out of 37 adenocarcinoma samples,

Statistical analysis was applied to verify the significance of these results, as described below.

The P value for the difference in the expression levels of solute carrier organic anion transporter family, member 4A1 (SLCO4A1) transcripts detectable by the above amplicon in colon cancer samples versus the normal tissue samples was determined by T test as 1.39E-07.

Threshold of 3 fold overexpression was found to differentiate between cancer and normal samples with P value of 1.97E-04 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: T23657seg29-32F forward primer (SEQ ID NO: 1361); and T23657seg29-32R reverse primer (SEQ ID NO: 1362).

The present invention also preferably encompasses any amplicon obtained through the use of any suitable primer pair; for example, for the above experiment, the following amplicon was obtained as a non-limiting illustrative example only of a suitable amplicon: T23657seg29-32 (SEQ ID NO: 1363).

Forward primer (SEQ ID NO: 1361):
CCTTTGCCCTGGGAATCC
Reverse primer (SEQ ID NO: 1362):
GCCCTGCTGGCCACAC
Amplicon (SEQ ID NO: 1363):
CCTTTGCCCTGGGAATCCAGTGGATTGTAGTTAGAATACTAGGGGGCATC
CCGGGGCCCATCGCCTTCGGCTGGGTGATCGACAAGGCCTGTCTGCTGTG
GCAGGACCAGTGTGGCCAGCAGGGC

### Expression of Solute Carrier Organic Anion Transporter Family, Member 4A1 (SLC04A1) T23657 Transcripts which are Detectable by Amplicon as Depicted in Sequence Name T23657 Seg41 (SEQ ID NO: 1366) in Normal and Cancerous Colon Tissues

Expression of solute carrier organic anion transporter family, member 4A1 (SLCO4A1) transcripts detectable by or according to seg41, T23657 amplicon (SEQ ID NO: 1366) and T23657 Seg41F (SEQ ID NO: 1364) T23657 Seg41 R (SEQ ID NO: 1365) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

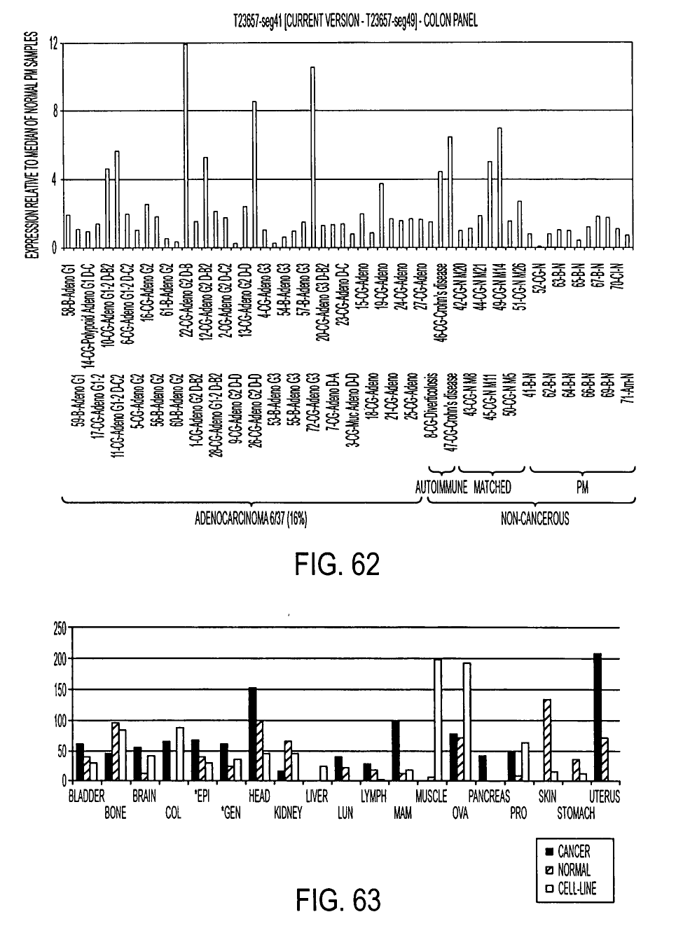

FIG. 62 is a histogram showing over expression of the above-indicated solute carrier organic anion transporter family, member 4A1 (SLCO4A1) transcripts in cancerous colon samples relative to the normal samples. The number and percentage of samples that exhibit at least 4 fold over-expression, out of the total number of samples tested is indicated in the bottom.

As is evident from FIG. 62, the expression of solute carrier organic anion transporter family, member 4A1 (SLCO4A1) transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 4 fold was found in 6 out of 37 adenocarcinoma samples,

Statistical analysis was applied to verify the significance of these results, as described below.

The P value for the difference in the expression levels of solute carrier organic anion transporter family, member 4A1 (SLCO4A1) transcripts detectable by the above amplicon in colon cancer samples versus the normal tissue samples was determined by T test as 3.02E-03.

Threshold of 4 fold overexpression was found to differentiate between cancer and normal samples with P value of 1.89E-01 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: T23657seg41F forward primer (SEQ ID NO: 1364); and T23657seg41R reverse primer (SEQ ID NO: 1365).

The present invention also preferably encompasses any amplicon obtained through the use of any suitable primer pair; for example, for the above experiment, the following amplicon was obtained as a non-limiting illustrative example only of a suitable amplicon: T23657seg41 (SEQ ID NO: 1366).

Forward primer (SEQ ID NO: 1364):
CCGTGATGGATGTGGAGTCTC
Reverse primer (SEQ ID NO: 1365):
GCATCGGAAGCAAATGCATT
Amplicon (SEQ ID NO: 1366):
CCGTGATGGATGTGGAGTCTCGGCTTTCTGACAACGTCTTCCAGAGCAGG
CTTTCTCTAGAGGGTGGACTGCCTGTGTTCTCCTGGGAGAGAATGCATTT
GCTTCCGATGC

### Description for Cluster T51958

Cluster T51958 features 12 transcript(s) and 48 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| T51958_PEA_1_T4 | 1081 |
| T51958_PEA_1_T5 | 1082 |
| T51958_PEA_1_T6 | 1083 |
| T51958_PEA_1_T8 | 1084 |
| T51958_PEA_1_T12 | 1085 |
| T51958_PEA_1_T16 | 1086 |
| T51958_PEA_1_T33 | 1087 |
| T51958_PEA_1_T35 | 1088 |
| T51958_PEA_1_T37 | 1089 |
| T51958_PEA_1_T39 | 1090 |
| T51958_PEA_1_T40 | 1091 |
| T51958_PEA_1_T41 | 1092 |

TABLE 2
Segments of interest

| Segment Name | SEQ ID NO: |
|---|---|
| T51958_PEA_1_node_0 | 1093 |
| T51958_PEA_1_node_7 | 1094 |
| T51958_PEA_1_node_8 | 1095 |
| T51958_PEA_1_node_9 | 1096 |
| T51958_PEA_1_node_14 | 1097 |
| T51958_PEA_1_node_16 | 1098 |
| T51958_PEA_1_node_18 | 1099 |
| T51958_PEA_1_node_21 | 1100 |
| T51958_PEA_1_node_22 | 1101 |
| T51958_PEA_1_node_24 | 1102 |
| T51958_PEA_1_node_27 | 1103 |
| T51958_PEA_1_node_29 | 1104 |
| T51958_PEA_1_node_33 | 1105 |
| T51958_PEA_1_node_40 | 1106 |
| T51958_PEA_1_node_41 | 1107 |
| T51958_PEA_1_node_46 | 1108 |
| T51958_PEA_1_node_51 | 1109 |
| T51958_PEA_1_node_55 | 1110 |
| T51958_PEA_1_node_67 | 1111 |
| T51958_PEA_1_node_70 | 1112 |
| T51958_PEA_1_node_74 | 1113 |
| T51958_PEA_1_node_78 | 1114 |
| T51958_PEA_1_node_11 | 1115 |
| T51958_PEA_1_node_15 | 1116 |
| T51958_PEA_1_node_20 | 1117 |
| T51958_PEA_1_node_26 | 1118 |
| T51958_PEA_1_node_35 | 1119 |
| T51958_PEA_1_node_36 | 1120 |
| T51958_PEA_1_node_38 | 1121 |
| T51958_PEA_1_node_39 | 1122 |
| T51958_PEA_1_node_42 | 1123 |
| T51958_PEA_1_node_43 | 1124 |
| T51958_PEA_1_node_44 | 1125 |
| T51958_PEA_1_node_45 | 1126 |
| T51958_PEA_1_node_47 | 1127 |
| T51958_PEA_1_node_48 | 1128 |
| T51958_PEA_1_node_49 | 1129 |
| T51958_PEA_1_node_50 | 1130 |
| T51958_PEA_1_node_54 | 1131 |
| T51958_PEA_1_node_61 | 1132 |
| T51958_PEA_1_node_71 | 1133 |
| T51958_PEA_1_node_72 | 1134 |
| T51958_PEA_1_node_75 | 1135 |
| T51958_PEA_1_node_76 | 1136 |
| T51958_PEA_1_node_77 | 1137 |
| T51958_PEA_1_node_80 | 1138 |
| T51958_PEA_1_node_82 | 1139 |
| T51958_PEA_1_node_84 | 1140 |

TABLE 3
Proteins of interest

| | SEQ | |
|---|---|---|
| | ID | |
| Protein Name | NO: | Corresponding Transcript(s) |
| T51958_PEA_1_P5 | 1151 | T51958_PEA_1_T4 (SEQ ID NO: |
| | | 1081); T51958_PEA_1_T12 (SEQ |
| | | ID NO: 1085); T51958_PEA_1_T16 |
| | | (SEQ ID NO: 1086); |
| | | T51958_PEA_1_T33 (SEQ ID NO: |
| | | 1087); T51958_PEA_1_T35 (SEQ |
| | | ID NO: 1088) |
| T51958_PEA_1_P6 | 1152 | T51958_PEA_1_T5 (SEQ ID NO: 1082) |
| T51958_PEA_1_P28 | 1153 | T51958_PEA_1_T37 (SEQ ID NO: |
| | | 1089); T51958_PEA_1_T39 (SEQ |
| | | ID NO: 1090) |
| T51958_PEA_1_P30 | 1154 | T51958_PEA_1_T40 (SEQ ID NO: |
| | | 1091) |
| T51958_PEA_1_P34 | 1155 | T51958_PEA_1_T8 (SEQ ID NO: 1084) |
| T51958_PEA_1_P35 | 1156 | T51958_PEA_1_T6 (SEQ ID NO: |
| | | 1083); T51958_PEA_1_T41 (SEQ |
| | | ID NO: 1092) |

These sequences are variants of the known protein Tyrosine-protein kinase-like 7 precursor (SwissProt accession identifier PTK7_HUMAN; known also according to the synonyms Colon carcinoma kinase-4; CCK4), SEQ ID NO:1141, referred to herein as the previously known protein.

Protein Tyrosine-protein kinase-like 7 precursor (SEQ ID NO:1141) is known or believed to have the following function(s): MAY FUNCTION AS A CELL ADHESION MOLECULE. LACKS PROBABLY THE CATALYTIC ACTIVITY OF TYROSINE KINASE. MAY BE CONNECTED TO THE PATHOPHYSIOLOGY OF COLON CARCINOMAS AND/OR MAY REPRESENT A TUMOR PROGRESSION MARKER. The sequence for protein Tyrosine-protein kinase-like 7 precursor is given at the end of the application, as “Tyrosine-protein kinase-like 7 precursor amino acid sequence”. Known polymorphisms for this sequence are as shown in Table 4.

TABLE 4
Amino acid mutations for Known Protein

| SNP position(s) on amino | |
|---|---|
| acid sequence | Comment |
| 92 | P -> R |
| 147 | K -> T |
| 207 | S -> G |
| 495-496 | VL -> RV |
| 515 | G -> E |
| 881 | E -> G |
| 969 | A -> P |
| 992 | S -> F |

Protein Tyrosine-protein kinase-like 7 precursor (SEQ ID NO:1141) localization is believed to be Type I membrane protein.

The following GO Annotation(s) apply to the previously known protein. The following annotation(s) were found: protein amino acid phosphorylation; cell adhesion; signal transduction, which are annotation(s) related to Biological Process; protein tyrosine kinase; transmembrane receptor protein tyrosine kinase; receptor; protein binding; ATP binding; transferase, which are annotation(s) related to Molecular Function; and integral plasma membrane protein, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster T51958 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 63 and Table 5. This cluster is overexpressed (at least at a minimum level) in the following pathological conditions: epithelial malignant tumors and a mixture of malignant tumors from different tissues.

TABLE 5
Normal tissue distribution

| Name of Tissue | Number |
|---|---|
| bladder | 41 |
| bone | 97 |
| brain | 12 |
| colon | 0 |
| epithelial | 40 |
| general | 24 |
| head and neck | 101 |
| kidney | 67 |
| liver | 0 |
| lung | 23 |
| lymph nodes | 18 |
| breast | 13 |
| muscle | 7 |
| ovary | 72 |
| pancreas | 0 |
| prostate | 8 |
| skin | 134 |
| stomach | 36 |
| uterus | 72 |

TABLE 6
P values and ratios for expression in cancerous tissue

| Name of Tissue | P1 | P2 | SP1 | R3 | SP2 | R4 |
|---|---|---|---|---|---|---|
| bladder | 5.4e−01 | 6.3e−01 | 6.0e−01 | 1.3 | 7.6e−01 | 1.0 |
| bone | 6.6e−01 | 5.5e−01 | 8.7e−01 | 0.6 | 8.1e−01 | 0.7 |
| brain | 3.1e−02 | 1.1e−02 | 2.8e−02 | 3.8 | 1.7e−02 | 3.4 |
| colon | 3.0e−02 | 2.5e−02 | 2.4e−01 | 3.1 | 1.6e−01 | 3.0 |
| epithelial | 6.2e−03 | 2.0e−02 | 1.8e−02 | 1.5 | 1.9e−01 | 1.1 |
| general | 7.2e−07 | 2.3e−06 | 4.4e−07 | 2.3 | 1.5e−05 | 1.9 |
| head and neck | 3.4e−01 | 3.3e−01 | 7.1e−01 | 1.3 | 8.4e−01 | 0.9 |
| kidney | 7.3e−01 | 5.8e−01 | 9.7e−01 | 0.4 | 9.3e−01 | 0.6 |
| liver | 1 | 6.8e−01 | 1 | 1.0 | 6.9e−01 | 1.4 |
| lung | 5.9e−01 | 8.4e−01 | 5.4e−01 | 1.5 | 8.4e−01 | 0.8 |
| lymph nodes | 5.1e−01 | 6.0e−01 | 1 | 1.3 | 1 | 0.8 |
| breast | 4.0e−01 | 3.0e−01 | 1.5e−01 | 2.5 | 3.1e−01 | 1.8 |
| muscle | 9.2e−01 | 4.8e−01 | 1 | 0.7 | 9.1e−03 | 2.9 |
| ovary | 3.7e−01 | 2.5e−01 | 7.1e−01 | 1.0 | 4.4e−01 | 1.2 |
| pancreas | 1.2e−01 | 2.1e−01 | 7.6e−02 | 5.1 | 1.5e−01 | 3.7 |
| prostate | 6.5e−01 | 5.4e−01 | 1.4e−01 | 2.5 | 1.0e−01 | 2.7 |
| skin | 7.7e−01 | 8.1e−01 | 1 | 0.1 | 1 | 0.2 |
| stomach | 5.8e−01 | 7.5e−01 | 1 | 0.5 | 9.6e−01 | 0.6 |
| uterus | 2.2e−01 | 5.6e−01 | 4.2e−02 | 1.8 | 3.7e−01 | 1.0 |

As noted above, cluster T51958 features 12 transcript(s), which were listed in Table 1 above. These transcript(s) encode for protein(s) which are variant(s) of protein Tyrosine-protein kinase-like 7 precursor (SEQ ID NO:1141). A description of each variant protein according to the present invention is now provided.

Variant protein T51958_PEA_{—}1_P5 (SEQ ID NO:1151) according to the present invention has an amino acid sequence as given at the end of the application; it is encoded by transcript(s) T51958_PEA_{—}1_T4 (SEQ ID NO:1081). An alignment is given to the known protein (Tyrosine-protein kinase-like 7 precursor (SEQ ID NO:1141)) at the end of the application. One or more alignments to one or more previously published protein sequences are given at the end of the application. A brief description of the relationship of the variant protein according to the present invention to each such aligned protein is as follows:

Comparison Report Between T51958_PEA_{—}1_P5 (SEQ ID NO:1151) and PTK7_HUMAN_V4 (SEQ ID NO:1143):

1. An isolated chimeric polypeptide encoding for T51958_PEA_{—}1_P5 (SEQ ID NO:1151), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQP QTQVTLRCHIDGHPRPTYQWFRDGTPLSDGQSNHTVSSKERNLTLRPAGPEHSGLYSCCAHSAFGQACSS QNFTLSIADESFARVVLAPQDVVVARYEEAMFHCQFSAQPPPSLQWLFEDETPITNRSRPPHLRRATVFAN GSLLLTQVRPRNAGIYRCIGQGQRGPPIILEATLHLAEIEDMPLFEPRVFTAGSEERVTCLPPKGLPEPSVWW EHAGVRLPTHGRVYQKGHELVLANIAESDAGVYTCHAANLAGQRRQDVNITVATVPSWLKKPQDSQLEE GKPGYLDCLTQATPKPTVVWYRNQMLISEDSRFEVFKNGTLRINSVEVYDGTWYRCMSSTPAGSIEAQAR VQVLEKLKFTPPPQPQQCMEFDKEATVPCSATGREKPTIKWERADGSSLPEWVTDNAGTLHFARVTRDDA GNYTCIASNGPQGQIRAHVQLTVAVFITFKVEPERTTVYQGHTALLQCEAQGDPKPLIQWKGKDRILDPTK LGPRMHIFQNGSLVIHDVAPEDSGRYTCIAGNSCNIKHTEAPLYVV corresponding to amino acids 1-682 of PTK7_HUMAN_V4 (SEQ ID NO:1143), which also corresponds to amino acids 1-682 of T51958_PEA_{—}1_P5 (SEQ ID NO:1151), and a second amino acid sequence being at least 70%, optionally at least 80%, preferably at least 85%, more preferably at least 90% and most preferably at least 95% homologous to a polypeptide having the sequence GMGWGGLCCTGSGGPRRLSPCTQPLCTEHGTEAIFVAAVGIRPSHHAAAQS (SEQ ID NO:1451) corresponding to amino acids 683-733 of T51958_PEA_{—}1_P5 (SEQ ID NO:1151), wherein said first amino acid sequence and second amino acid sequence are contiguous and in a sequential order.

2. An isolated polypeptide encoding for a tail of T51958_PEA_{—}1_P5 (SEQ ID NO:1151), comprising a polypeptide being at least 70%, optionally at least about 80%, preferably at least about 85%, more preferably at least about 90% and most preferably at least about 95% homologous to the sequence GMGWGGLCCTGSGGPRRLSPCTQPLCTEHGTEAIFVAAVGIRPSHHAAAQS (SEQ ID NO:1451) in T51958_PEA_{—}1_P5 (SEQ ID NO:1151).

It should be noted that the known protein sequence (PTK7_HUMAN (SEQ ID NO:1141)) has one or more changes than the sequence given at the end of the application and named as being the amino acid sequence for PTK7_HUMAN_V4 (SEQ ID NO:1143). These changes were previously known to occur and are listed in the table below.

TABLE 7
Changes to PTK7_HUMAN_V4 (SEQ ID NO: 1143)

| SNP position(s) on amino | |
|---|---|
| acid sequence | Type of change |
| 93 | conflict |
| 148 | conflict |
| 208 | conflict |
| 496 | conflict |
| 516 | conflict |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein T51958_PEA_{—}1_P5 (SEQ ID NO:1151) is encoded by the following transcript(s): T51958_PEA_{—}1_T4 (SEQ ID NO:1081), for which the sequence(s) is/are given at the end of the application. The coding portion of transcript T51958_PEA_{—}1_T4 (SEQ ID NO:1081) is shown in bold; this coding portion starts at position 209 and ends at position 2407. The transcript also has the following SNPs as listed in Table 8 (given according to their position on the nucleotide sequence, with the alternative nucleic acid listed; the last column indicates whether the SNP is known or not; the presence of known SNPs in variant protein T51958_PEA_{—}1_P5 (SEQ ID NO:1151) sequence provides support for the deduced sequence of this variant protein according to the present invention).

TABLE 8
Nucleic acid SNPs

| SNP position on | Alternative | Previously |
|---|---|---|
| nucleotide sequence | nucleic acid | known SNP? |
| 2059 | G -> A | No |
| 3778 | T -> A | No |
| 3799 | C -> | No |
| 4638 | C -> | No |
| 4771 | T -> C | No |
| 4979 | G -> C | No |

Variant protein T51958_PEA_{—}1_P6 (SEQ ID NO:1152) according to the present invention has an amino acid sequence as given at the end of the application; it is encoded by transcript(s) T51958_PEA_{—}1_T5 (SEQ ID NO:1082). An alignment is given to the known protein (Tyrosine-protein kinase-like 7 precursor (SEQ ID NO:1141)) at the end of the application. One or more alignments to one or more previously published protein sequences are given at the end of the application. A brief description of the relationship of the variant protein according to the present invention to each such aligned protein is as follows:

Comparison Report Between T51958_PEA_{—}1_P6 (SEQ ID NO:1152) and PTK7_HUMAN_V4 (SEQ ID NO:1143):

1. An isolated chimeric polypeptide encoding for T51958_PEA_{—}1_P6 (SEQ ID NO:1152), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQP QTQVTLRCHIDGHPRPTYQWFRDGTPLSDGQSNHTVSSKERNLTLRPAGPEHSGLYSCCAHSAFGQACSS QNFTLSIADESFARVVLAPQDVVVARYEEAMFHCQFSAQPPPSLQWLFEDETPITNRSRPPHLRRATVFAN GSLLLTQVRPRNAGIYRCIGQGQRGPPIILEATLHLAEIEDMPLFEPRVFTAGSEERVTCLPPKGLPEPSVWW EHAGVRLPTHGRVYQKGHELVLANIAESDAGVYTCHAANLAGQRRQDVNITVATVPSWLKKPQDSQLEE GKPGYLDCLTQATPKPTVVWYRNQMLISEDSRFEVFKNGTLRINSVEVYDGTWYRCMSSTPAGSIEAQAR VQVLEKLKFTPPPQPQQCMEFDKEATVPCSATGREKPTIKWERADGSSLPEWVTDNAGTLHFARVTRDDA GNYTCIASNGPQGQIRAHVQLTVAVFITFKVEPERTTVYQGHTALLQCEAQGDPKPLIQWKGKDRILDPTK LGPRM corresponding to amino acids 1-641 of PTK7_HUMAN_V4 (SEQ ID NO:1143), which also corresponds to amino acids 1-641 of T51958_PEA_{—}1_P6 (SEQ ID NO:1152), and a second amino acid sequence being at least 70%, optionally at least 80%, preferably at least 85%, more preferably at least 90% and most preferably at least 95% homologous to a polypeptide having the sequence APW corresponding to amino acids 642-644 of T51958_PEA_{—}1_P6 (SEQ ID NO:1152), wherein said first amino acid sequence and second amino acid sequence are contiguous and in a sequential order.

It should be noted that the known protein sequence (PTK7_HUMAN (SEQ ID NO:1141)) has one or more changes than the sequence given at the end of the application and named as being the amino acid sequence for PTK7_HUMAN_V4 (SEQ ID NO:1143). These changes were previously known to occur and are listed in the table below.

TABLE 9
Changes to PTK7_HUMAN_V4 (SEQ ID NO: 1143)

| SNP position(s) on amino | |
|---|---|
| acid sequence | Type of change |
| 93 | conflict |
| 148 | conflict |
| 208 | conflict |
| 496 | conflict |
| 516 | conflict |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein T51958_PEA_{—}1_P6 (SEQ ID NO:1152) is encoded by the following transcript(s): T51958_PEA_{—}1_T5 (SEQ ID NO:1082), for which the sequence(s) is/are given at the end of the application. The coding portion of transcript T51958_PEA_{—}1_T5 (SEQ ID NO:1082) is shown in bold; this coding portion starts at position 209 and ends at position 2140. The transcript also has the following SNPs as listed in Table 10 (given according to their position on the nucleotide sequence, with the alternative nucleic acid listed; the last column indicates whether the SNP is known or not; the presence of known SNPs in variant protein T51958_PEA_{—}1_P6 (SEQ ID NO:1152) sequence provides support for the deduced sequence of this variant protein according to the present invention).

TABLE 10
Nucleic acid SNPs

| SNP position on | Alternative | Previously |
|---|---|---|
| nucleotide sequence | nucleic acid | known SNP? |
| 2059 | G -> A | No |
| 3762 | T -> A | No |
| 3783 | C -> | No |
| 4622 | C -> | No |
| 4755 | T -> C | No |
| 4963 | G -> C | No |

Variant protein T51958_PEA_{—}1_P28 (SEQ ID NO:1153) according to the present invention has an amino acid sequence as given at the end of the application; it is encoded by transcript(s) T51958_PEA_{—}1_T37 (SEQ ID NO:1089). An alignment is given to the known protein (Tyrosine-protein kinase-like 7 precursor (SEQ ID NO:1141)) at the end of the application. One or more alignments to one or more previously published protein sequences are given at the end of the application. A brief description of the relationship of the variant protein according to the present invention to each such aligned protein is as follows:

Comparison Report Between T51958_PEA_{—}1_P28 (SEQ ID NO:1153) and PTK7_HUMAN_V11 (SEQ ID NO:1144):

1. An isolated chimeric polypeptide encoding for T51958_PEA_{—}1_P28 (SEQ ID NO:1153), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQP QTQVTLRCHIDGHPRPTYQWFRDGTPLSDGQSNHTVSSKERNLTLRPAGPEHSGLYSCCAHSAFGQACSS QNFTLSIADESFARVVLAPQDVVVARYEEAMFHCQFSAQPPPSLQWLFEDETPITNRSRPPHLRRATVFAN GSLLLTQVRPRNAGIYRCIGQGQRGPPIILEATLHLAEIEDMPLFEPRVFTAGSEERVTCLPPKGLPEPSVWW EHAGVRLPTHGRVYQKGHELVLANIAESDAGVYTCHAANLAGQRRQDVNITVA corresponding to amino acids 1-409 of PTK7_HUMAN_V11 (SEQ ID NO:1144), which also corresponds to amino acids 1-409 of T51958_PEA_{—}1_P28 (SEQ ID NO:1153), and a second amino acid sequence being at least 70%, optionally at least 80%, preferably at least 85%, more preferably at least 90% and most preferably at least 95% homologous to a polypeptide having the sequence SEHLCPEGQGEVEGNTGLGVMDRGFPGTHLRSSQFWALQAWESVHYWESV corresponding to amino acids 410-459 of T51958_PEA_{—}1_P28 (SEQ ID NO:1153), wherein said first amino acid sequence and second amino acid sequence are contiguous and in a sequential order.

2. An isolated polypeptide encoding for a tail of T51958_PEA_{—}1_P28 (SEQ ID NO:1153), comprising a polypeptide being at least 70%, optionally at least about 80%, preferably at least about 85%, more preferably at least about 90% and most preferably at least about 95% homologous to the sequence SEHLCPEGQGEVEGNTGLGVMDRGFPGTHLRSSQFWALQAWESVHYWESV in T51958_PEA_{—}1_P28 (SEQ ID NO:1153).

It should be noted that the known protein sequence (PTK7_HUMAN (SEQ ID NO:1141)) has one or more changes than the sequence given at the end of the application and named as being the amino acid sequence for PTK7_HUMAN_V11 (SEQ ID NO:1144). These changes were previously known to occur and are listed in the table below.

TABLE 11
Changes to PTK7_HUMAN_V11 (SEQ ID NO: 1144)

| SNP position(s) on amino | |
|---|---|
| acid sequence | Type of change |
| 93 | conflict |
| 148 | conflict |
| 208 | conflict |

### Comparison Report Between T51958_PEA_{—}1_P28 (SEQ ID NO:1153) and Q8NFA5 (SEQ ID NO:1147):

1. An isolated chimeric polypeptide encoding for T51958_PEA_{—}1_P28 (SEQ ID NO:1153), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQP QTQVTLRCHIDGHPRPTYQWFRDGTPLSDGQSNHTVSSKERNLTLRPAGPEHSGLYSCCAHSAFGQACSS QNFTLSIADESFARVVLAPQDVVVARYEEAMFHCQFSAQPPPSLQWLFEDETPITNRSRPPHLRRATVFAN GSLLLTQVRPRNAGIYRCIGQGQRGPPIILEATLHLAEIEDMPLFEPRVFTAGSEERVTCLPPKGLPEPSVWW EHAGVRLPTHGRVYQKGHELVLANIAESDAGVYTCHAANLAGQRRQDVNITVA corresponding to amino acids 1-409 of Q8NFA5 (SEQ ID NO:1147), which also corresponds to amino acids 1-409 of T51958_PEA_{—}1_P28 (SEQ ID NO:1153), and a second amino acid sequence being at least 70%, optionally at least 80%, preferably at least 85%, more preferably at least 90% and most preferably at least 95% homologous to a polypeptide having the sequence SEHLCPEGQGEVEGNTGLGVMDRGFPGTHLRSSQFWALQAWESVHYWESV corresponding to amino acids 410-459 of T51958_PEA_{—}1_P28 (SEQ ID NO:1153), wherein said first amino acid sequence and second amino acid sequence are contiguous and in a sequential order.

2. An isolated polypeptide encoding for a tail of T51958_PEA_{—}1_P28 (SEQ ID NO:1153), comprising a polypeptide being at least 70%, optionally at least about 80%, preferably at least about 85%, more preferably at least about 90% and most preferably at least about 95% homologous to the sequence SEHLCPEGQGEVEGNTGLGVMDRGFPGTHLRSSQFWALQAWESVHYWESV in T51958_PEA_{—}1_P28 (SEQ ID NO:1153).

### Comparison Report Between T51958_PEA_{—}1_P28 (SEQ ID NO:1153) and Q8NFA6 (SEQ ID NO:1149):

1. An isolated chimeric polypeptide encoding for T51958_PEA_{—}1_P28 (SEQ ID NO:1153), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQP QTQVTLRCHIDGHPRPTYQWFRDGTPLSDGQSNHTVSSKERNLTLRPAGPEHSGLYSCCAHSAFGQACSS QNFTLSIADESFARVVLAPQDVVVARYEEAMFHCQFSAQPPPSLQWLFEDETPITNRSRPPHLRRATVFAN GSLLLTQVRPRNAGIYRCIGQGQRGPPIILEATLHLAEIEDMPLFEPRVFTAGSEERVTCLPPKGLPEPSVWW EHAGVRLPTHGRVYQKGHELVLANIAESDAGVYTCHAANLAGQRRQDVNITVA corresponding to amino acids 1-409 of Q8NFA6 (SEQ ID NO:1149), which also corresponds to amino acids 1-409 of T51958_PEA_{—}1_P28 (SEQ ID NO:1153), and a second amino acid sequence being at least 70%, optionally at least 80%, preferably at least 85%, more preferably at least 90% and most preferably at least 95% homologous to a polypeptide having the sequence SEHLCPEGQGEVEGNTGLGVMDRGFPGTHLRSSQFWALQAWESVHYWESV corresponding to amino acids 410-459 of T51958_PEA_{—}1_P28 (SEQ ID NO:1153), wherein said first amino acid sequence and second amino acid sequence are contiguous and in a sequential order.

2. An isolated polypeptide encoding for a tail of T51958_PEA_{—}1_P28 (SEQ ID NO:1153), comprising a polypeptide being at least 70%, optionally at least about 80%, preferably at least about 85%, more preferably at least about 90% and most preferably at least about 95% homologous to the sequence SEHLCPEGQGEVEGNTGLGVMDRGFPGTHLRSSQFWALQAWESVHYWESV in T51958_PEA_{—}1_P28 (SEQ ID NO:1153).

Comparison Report Between T51958_PEA_{—}1_P28 (SEQ ID NO:1153) and Q8NFA7 (SEQ ID NO:1148):

1. An isolated chimeric polypeptide encoding for T51958_PEA_{—}1_P28 (SEQ ID NO:1153), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQP QTQVTLRCHIDGHPRPTYQWFRDGTPLSDGQSNHTVSSKERNLTLRPAGPEHSGLYSCCAHSAFGQACSS QNFTLSIADESFARVVLAPQDVVVARYEEAMFHCQFSAQPPPSLQWLFEDETPITNRSRPPHLRRATVFAN GSLLLTQVRPRNAGIYRCIGQGQRGPPIILEATLHLAEIEDMPLFEPRVFTAGSEERVTCLPPKGLPEPSVWW EHAGVRLPTHGRVYQKGHELVLANIAESDAGVYTCHAANLAGQRRQDVNITVA corresponding to amino acids 1-409 of Q8NFA7 (SEQ ID NO:1148), which also corresponds to amino acids 1-409 of T51958_PEA_{—}1_P28 (SEQ ID NO:1153), and a second amino acid sequence being at least 70%, optionally at least 80%, preferably at least 85%, more preferably at least 90% and most preferably at least 95% homologous to a polypeptide having the sequence SEHLCPEGQGEVEGNTGLGVMDRGFPGTHLRSSQFWALQAWESVHYWESV corresponding to amino acids 410-459 of T51958_PEA_{—}1_P28 (SEQ ID NO:1153), wherein said first amino acid sequence and second amino acid sequence are contiguous and in a sequential order.

2. An isolated polypeptide encoding for a tail of T51958_PEA_{—}1_P28 (SEQ ID NO:1153), comprising a polypeptide being at least 70%, optionally at least about 80%, preferably at least about 85%, more preferably at least about 90% and most preferably at least about 95% homologous to the sequence SEHLCPEGQGEVEGNTGLGVMDRGFPGTHLRSSQFWALQAWESVHYWESV in T51958_PEA_{—}1_P28 (SEQ ID NO:1153).

Comparison Report Between T51958_PEA_{—}1_P28 (SEQ ID NO:1153) and Q8NFA8 (SEQ ID NO:1146):

1. An isolated chimeric polypeptide encoding for T51958_PEA_{—}1_P28 (SEQ ID NO:1153), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQP QTQVTLRCHIDGHPRPTYQWFRDGTPLSDGQSNHTVSSKERNLTLRPAGPEHSGLYSCCAHSAFGQACSS QNFTLSIADESFARVVLAPQDVVVARYEEAMFHCQFSAQPPPSLQWLFEDETPITNRSRPPHLRRATVFAN GSLLLTQVRPRNAGIYRCIGQGQRGPPIILEATLHLAEIEDMPLFEPRVFTAGSEERVTCLPPKGLPEPSVWW EHAGVRLPTHGRVYQKGHELVLANIAESDAGVYTCHAANLAGQRRQDVNITVA corresponding to amino acids 1-409 of Q8NFA8 (SEQ ID NO:1146), which also corresponds to amino acids 1-409 of T51958_PEA_{—}1_P28 (SEQ ID NO:1153), and a second amino acid sequence being at least 70%, optionally at least 80%, preferably at least 85%, more preferably at least 90% and most preferably at least 95% homologous to a polypeptide having the sequence SEHLCPEGQGEVEGNTGLGVMDRGFPGTHLRSSQFWALQAWESVHYWESV corresponding to amino acids 410-459 of T51958_PEA_{—}1_P28 (SEQ ID NO:1153), wherein said first amino acid sequence and second amino acid sequence are contiguous and in a sequential order.

2. An isolated polypeptide encoding for a tail of T51958_PEA_{—}1_P28 (SEQ ID NO:1153), comprising a polypeptide being at least 70%, optionally at least about 80%, preferably at least about 85%, more preferably at least about 90% and most preferably at least about 95% homologous to the sequence SEHLCPEGQGEVEGNTGLGVMDRGFPGTHLRSSQFWALQAWESVHYWESV in T51958_PEA_{—}1_P28 (SEQ ID NO:1153).

Comparison Report Between T51958_PEA_{—}1_P28 (SEQ ID NO:1153) and AAN04862 (SEQ ID NO:1150) (SEQ ID NO:1150):

1. An isolated chimeric polypeptide encoding for T51958_PEA_{—}1_P28 (SEQ ID NO:1153), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQP QTQVTLRCHIDGHPRPTYQWFRDGTPLSDGQSNHTVSSKERNLTLRPAGPEHSGLYSCCAHSAFGQACSS QNFTLSIADESFARVVLAPQDVVVARYEEAMFHCQFSAQPPPSLQWLFEDETPITNRSRPPHLRRATVFAN GSLLLTQVRPRNAGIYRCIGQGQRGPPIILEATLHLAEIEDMPLFEPRVFTAGSEERVTCLPPKGLPEPSVWW EHAGVRLPTHGRVYQKGHELVLANIAESDAGVYTCHAANLAGQRRQDVNITVA corresponding to amino acids 1-409 of AAN04862 (SEQ ID NO:1150), which also corresponds to amino acids 1-409 of T51958_PEA_{—}1_P28 (SEQ ID NO:1153), and a second amino acid sequence being at least 70%, optionally at least 80%, preferably at least 85%, more preferably at least 90% and most preferably at least 95% homologous to a polypeptide having the sequence SEHLCPEGQGEVEGNTGLGVMDRGFPGTHLRSSQFWALQAWESVHYWESV corresponding to amino acids 410-459 of T51958_PEA_{—}1_P28 (SEQ ID NO:1153), wherein said first amino acid sequence and second amino acid sequence are contiguous and in a sequential order.

2. An isolated polypeptide encoding for a tail of T51958_PEA_{—}1_P28 (SEQ ID NO:1153), comprising a polypeptide being at least 70%, optionally at least about 80%, preferably at least about 85%, more preferably at least about 90% and most preferably at least about 95% homologous to the sequence SEHLCPEGQGEVEGNTGLGVMDRGFPGTHLRSSQFWALQAWESVHYWESV in T51958_PEA_{—}1_P28 (SEQ ID NO:1153).

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein T51958_PEA_{—}1_P28 (SEQ ID NO:1153) is encoded by the following transcript(s): T51958_PEA_{—}1_T37 (SEQ ID NO:1089), for which the sequence(s) is/are given at the end of the application. The coding portion of transcript T51958_PEA_{—}1_T37 (SEQ ID NO:1089) is shown in bold; this coding portion starts at position 209 and ends at position 1585.

Variant protein T51958_PEA_{—}1_P30 (SEQ ID NO:1154) according to the present invention has an amino acid sequence as given at the end of the application; it is encoded by transcript(s) T51958_PEA_{—}1_T40 (SEQ ID NO:1091). An alignment is given to the known protein (Tyrosine-protein kinase-like 7 precursor (SEQ ID NO:1141)) at the end of the application. One or more alignments to one or more previously published protein sequences are given at the end of the application. A brief description of the relationship of the variant protein according to the present invention to each such aligned protein is as follows:

Comparison Report Between T51958_PEA_{—}1_P30 (SEQ ID NO:1154) and PTK7_HUMAN_V13 (SEQ ID NO:1145) (SEQ ID NO:1145):

1. An isolated chimeric polypeptide encoding for T51958_PEA_{—}1_P30 (SEQ ID NO:1154), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIK corresponding to amino acids 1-122 of PTK7_HUMAN_V13 (SEQ ID NO:1145), which also corresponds to amino acids 1-122 of T51958_PEA_{—}1_P30 (SEQ ID NO:1154), and a second amino acid sequence being at least 70%, optionally at least 80%, preferably at least 85%, more preferably at least 90% and most preferably at least 95% homologous to a polypeptide having the sequence CESQGGCAQSPCQTLND (SEQ ID NO:1453) corresponding to amino acids 123-139 of T51958_PEA_{—}1_P30 (SEQ ID NO:1154), wherein said first amino acid sequence and second amino acid sequence are contiguous and in a sequential order.

2. An isolated polypeptide encoding for a tail of T51958_PEA_{—}1_P30 (SEQ ID NO:1154), comprising a polypeptide being at least 70%, optionally at least about 80%, preferably at least about 85%, more preferably at least about 90% and most preferably at least about 95% homologous to the sequence CESQGGCAQSPCQTLND (SEQ ID NO:1453) in T51958_PEA_{—}1_P30 (SEQ ID NO:1154).

It should be noted that the known protein sequence (PTK7_HUMAN (SEQ ID NO:1141)) has one or more changes than the sequence given at the end of the application and named as being the amino acid sequence for PTK7_HUMAN_V13 (SEQ ID NO:1145). These changes were previously known to occur and are listed in the table below.

TABLE 12
Changes to PTK7_HUMAN_V13 (SEQ ID NO: 1145)

| SNP position(s) on amino | |
|---|---|
| acid sequence | Type of change |
| 93 | conflict |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein T51958_PEA_{—}1_P30 (SEQ ID NO:1154) is encoded by the following transcript(s): T51958_PEA_{—}1_T40 (SEQ ID NO:1091), for which the sequence(s) is/are given at the end of the application. The coding portion of transcript T51958_PEA_{—}1_T40 (SEQ ID NO:1091) is shown in bold; this coding portion starts at position 209 and ends at position 625.

Variant protein T51958_PEA_{—}1_P34 (SEQ ID NO:1155) according to the present invention has an amino acid sequence as given at the end of the application; it is encoded by transcript(s) T51958_PEA_{—}1_T8 (SEQ ID NO:1084). An alignment is given to the known protein (Tyrosine-protein kinase-like 7 precursor (SEQ ID NO:1141)) at the end of the application. One or more alignments to one or more previously published protein sequences are given at the end of the application. A brief description of the relationship of the variant protein according to the present invention to each such aligned protein is as follows:

Comparison Report Between T51958_PEA_{—}1_P34 (SEQ ID NO:1155) and PTK7_HUMAN_V3 (SEQ ID NO:1142):

1. An isolated chimeric polypeptide encoding for T51958_PEA_{—}1_P34 (SEQ ID NO:1155), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQP QTQVTLRCHIDGHPR corresponding to amino acids 1-157 of PTK7_HUMAN_V3 (SEQ ID NO:1142), which also corresponds to amino acids 1-157 of T51958_PEA_{—}1_P34 (SEQ ID NO:1155).

It should be noted that the known protein sequence (PTK7_HUMAN (SEQ ID NO:1141)) has one or more changes than the sequence given at the end of the application and named as being the amino acid sequence for PTK7_HUMAN_V3 (SEQ ID NO:1142). These changes were previously known to occur and are listed in the table below.

TABLE 13
Changes to PTK7_HUMAN_V3 (SEQ ID NO: 1142)

| SNP position(s) on amino | |
|---|---|
| acid sequence | Type of change |
| 93 | conflict |
| 148 | conflict |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein T51958_PEA_{—}1_P34 (SEQ ID NO:1155) is encoded by the following transcript(s): T51958_PEA_{—}1_T8 (SEQ ID NO:1084), for which the sequence(s) is/are given at the end of the application. The coding portion of transcript T51958_PEA_{—}1_T8 (SEQ ID NO:1084) is shown in bold; this coding portion starts at position 209 and ends at position 679. The transcript also has the following SNPs as listed in Table 14 (given according to their position on the nucleotide sequence, with the alternative nucleic acid listed; the last column indicates whether the SNP is known or not; the presence of known SNPs in variant protein T51958_PEA_{—}1_P34 (SEQ ID NO:1155) sequence provides support for the deduced sequence of this variant protein according to the present invention).

TABLE 14
Nucleic acid SNPs

| SNP position on | Alternative | Previously |
|---|---|---|
| nucleotide sequence | nucleic acid | known SNP? |
| 1868 | G -> A | No |
| 2465 | T -> A | No |
| 2486 | C -> | No |
| 3325 | C -> | No |
| 3458 | T -> C | No |
| 3666 | G -> C | No |

Variant protein T51958_PEA_{—}1_P35 (SEQ ID NO:1156) according to the present invention has an amino acid sequence as given at the end of the application; it is encoded by transcript(s) T51958_PEA_{—}1_T6 (SEQ ID NO:1083). An alignment is given to the known protein (Tyrosine-protein kinase-like 7 precursor (SEQ ID NO:1141)) at the end of the application. One or more alignments to one or more previously published protein sequences are given at the end of the application. A brief description of the relationship of the variant protein according to the present invention to each such aligned protein is as follows:

Comparison Report Between T51958_PEA_{—}1_P35 (SEQ ID NO:1156) and PTK7_HUMAN_V11 (SEQ ID NO:1144):

1. An isolated chimeric polypeptide encoding for T51958_PEA_{—}1_P35 (SEQ ID NO:1156), comprising a first amino acid sequence being at least 90% homologous to MGAARGSPARPRRLPLLSVLLLPLLGGTQTAIVFIKQPSSQDALQGRRALLRCEVEAPGPVHVYWLLDGAP VQDTERRFAQGSSLSFAAVDRLQDSGTFQCVARDDVTGEEARSANASFNIKWIEAGPVVLKHPASEAEIQP QTQVTLRCHIDGHPRPTYQWFRDGTPLSDGQSNHTVSSKERNLTLRPAGPEHSGLYSCCAHSAFGQACSS QNFTLSIA corresponding to amino acids 1-220 of PTK7_HUMAN_V11 (SEQ ID NO:1144), which also corresponds to amino acids 1-220 of T51958_PEA_{—}1_P35 (SEQ ID NO:1156), and a second amino acid sequence being at least 70%, optionally at least 80%, preferably at least 85%, more preferably at least 90% and most preferably at least 95% homologous to a polypeptide having the sequence GEPGVGAEGMR (SEQ ID NO:1454) corresponding to amino acids 221-231 of T51958_PEA_{—}1_P35 (SEQ ID NO:1156), wherein said first amino acid sequence and second amino acid sequence are contiguous and in a sequential order.

2. An isolated polypeptide encoding for a tail of T51958_PEA_{—}1_P35 (SEQ ID NO:1156), comprising a polypeptide being at least 70%, optionally at least about 80%, preferably at least about 85%, more preferably at least about 90% and most preferably at least about 95% homologous to the sequence GEPGVGAEGMR (SEQ ID NO:1454) in T51958_PEA_{—}1_P35 (SEQ ID NO:1156).

It should be noted that the known protein sequence (PTK7_HUMAN (SEQ ID NO:1141)) has one or more changes than the sequence given at the end of the application and named as being the amino acid sequence for PTK7_HUMAN_V11 (SEQ ID NO:1144). These changes were previously known to occur and are listed in the table below.

TABLE 15
Changes to PTK7_HUMAN_V11 (SEQ ID NO: 1144)

| SNP position(s) on amino | |
|---|---|
| acid sequence | Type of change |
| 93 | conflict |
| 148 | conflict |
| 208 | conflict |

The location of the variant protein was determined according to results from a number of different software programs and analyses, including analyses from SignalP and other specialized programs. The variant protein is believed to be located as follows with regard to the cell: secreted. The protein localization is believed to be secreted because both signal-peptide prediction programs predict that this protein has a signal peptide, and neither trans-membrane region prediction program predicts that this protein has a trans-membrane region.

Variant protein T51958_PEA_{—}1_P35 (SEQ ID NO:1156) is encoded by the following transcript(s): T51958_PEA_{—}1_T6 (SEQ ID NO:1083), for which the sequence(s) is/are given at the end of the application. The coding portion of transcript T51958_PEA_{—}1_T6 (SEQ ID NO:1083) is shown in bold; this coding portion starts at position 209 and ends at position 901. The transcript also has the following SNPs as listed in Table 16 (given according to their position on the nucleotide sequence, with the alternative nucleic acid listed; the last column indicates whether the SNP is known or not; the presence of known SNPs in variant protein T51958_PEA_{—}1_P35 (SEQ ID NO:1156) sequence provides support for the deduced sequence of this variant protein according to the present invention).

TABLE 16
Nucleic acid SNPs

| SNP position on | Alternative | Previously |
|---|---|---|
| nucleotide sequence | nucleic acid | known SNP? |
| 2149 | G -> A | No |
| 2751 | T -> A | No |
| 2772 | C -> | No |
| 3611 | C -> | No |
| 3744 | T -> C | No |
| 3952 | G -> C | No |

As noted above, cluster T51958 features 48 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster T51958_PEA_{—}1_node_{—}0 (SEQ ID NO:1093) according to the present invention is supported by 21 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088), T51958_PEA_{—}1_T37 (SEQ ID NO:1089), T51958_PEA_{—}1_T39 (SEQ ID NO:1090), T51958_PEA_{—}1_T40 (SEQ ID NO:1091) and T51958_PEA_{—}1_T41 (SEQ ID NO:1092). Table 17 below describes the starting and ending position of this segment on each transcript.

TABLE 17
Segment location on transcripts

| | Segment | Segment |
|---|---|---|
| | starting | ending |
| Transcript name | position | position |
| T51958_PEA_1_T4 (SEQ ID NO: 1081) | 1 | 287 |
| T51958_PEA_1_T5 (SEQ ID NO: 1082) | 1 | 287 |
| T51958_PEA_1_T6 (SEQ ID NO: 1083) | 1 | 287 |
| T51958_PEA_1_T8 (SEQ ID NO: 1084) | 1 | 287 |
| T51958_PEA_1_T12 (SEQ ID NO: 1085) | 1 | 287 |
| T51958_PEA_1_T16 (SEQ ID NO: 1086) | 1 | 287 |
| T51958_PEA_1_T33 (SEQ ID NO: 1087) | 1 | 287 |
| T51958_PEA_1_T35 (SEQ ID NO: 1088) | 1 | 287 |
| T51958_PEA_1_T37 (SEQ ID NO: 1089) | 1 | 287 |
| T51958_PEA_1_T39 (SEQ ID NO: 1090) | 1 | 287 |
| T51958_PEA_1_T40 (SEQ ID NO: 1091) | 1 | 287 |
| T51958_PEA_1_T41 (SEQ ID NO: 1092) | 1 | 287 |

Segment cluster T51958_PEA_{—}1_node_{—}7 (SEQ ID NO:1094) according to the present invention is supported by 29 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088), T51958_PEA_{—}1_T37 (SEQ ID NO:1089), T51958_PEA_{—}1_T39 (SEQ ID NO:1090), T51958_PEA_{—}1_T40 (SEQ ID NO:1091) and T51958_PEA_{—}1_T41 (SEQ ID NO:1092). Table 18 below describes the starting and ending position of this segment on each transcript.

TABLE 18
Segment location on transcripts

| | Segment | |
|---|---|---|
| | starting | Segment |
| Transcript name | position | ending position |
| T51958_PEA_1_T4 (SEQ ID NO: 1081) | 288 | 451 |
| T51958_PEA_1_T5 (SEQ ID NO: 1082) | 288 | 451 |
| T51958_PEA_1_T6 (SEQ ID NO: 1083) | 288 | 451 |
| T51958_PEA_1_T8 (SEQ ID NO: 1084) | 288 | 451 |
| T51958_PEA_1_T12 (SEQ ID NO: 1085) | 288 | 451 |
| T51958_PEA_1_T16 (SEQ ID NO: 1086) | 288 | 451 |
| T51958_PEA_1_T33 (SEQ ID NO: 1087) | 288 | 451 |
| T51958_PEA_1_T35 (SEQ ID NO: 1088) | 288 | 451 |
| T51958_PEA_1_T37 (SEQ ID NO: 1089) | 288 | 451 |
| T51958_PEA_1_T39 (SEQ ID NO: 1090) | 288 | 451 |
| T51958_PEA_1_T40 (SEQ ID NO: 1091) | 288 | 451 |
| T51958_PEA_1_T41 (SEQ ID NO: 1092) | 288 | 451 |

Segment cluster T51958_PEA_{—}1_node_{—}8 (SEQ ID NO:1095) according to the present invention is supported by 28 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088), T51958_PEA_{—}1_T37 (SEQ ID NO:1089), T51958_PEA_{—}1_T39 (SEQ ID NO:1090), T51958_PEA_{—}1_T40 (SEQ ID NO:1091) and T51958_PEA_{—}1_T41 (SEQ ID NO:1092). Table 19 below describes the starting and ending position of this segment on each transcript.

TABLE 19
Segment location on transcripts

| | Segment | |
|---|---|---|
| | starting | Segment |
| Transcript name | position | ending position |
| T51958_PEA_1_T4 (SEQ ID NO: 1081) | 452 | 575 |
| T51958_PEA_1_T5 (SEQ ID NO: 1082) | 452 | 575 |
| T51958_PEA_1_T6 (SEQ ID NO: 1083) | 452 | 575 |
| T51958_PEA_1_T8 (SEQ ID NO: 1084) | 452 | 575 |
| T51958_PEA_1_T12 (SEQ ID NO: 1085) | 452 | 575 |
| T51958_PEA_1_T16 (SEQ ID NO: 1086) | 452 | 575 |
| T51958_PEA_1_T33 (SEQ ID NO: 1087) | 452 | 575 |
| T51958_PEA_1_T35 (SEQ ID NO: 1088) | 452 | 575 |
| T51958_PEA_1_T37 (SEQ ID NO: 1089) | 452 | 575 |
| T51958_PEA_1_T39 (SEQ ID NO: 1090) | 452 | 575 |
| T51958_PEA_1_T40 (SEQ ID NO: 1091) | 452 | 575 |
| T51958_PEA_1_T41 (SEQ ID NO: 1092) | 452 | 575 |

Segment cluster T51958_PEA_{—}1_node_{—}9 (SEQ ID NO:1096) according to the present invention is supported by 4 libraries. The number of libraries was determined as previously described. This segment can be found in the following transcript(s): T51958_PEA_{—}1_T40 (SEQ ID NO:1091). Table 20 below describes the starting and ending position of this segment on each transcript.

TABLE 20
Segment location on transcripts

| | Segment | |
|---|---|---|
| | starting | Segment |
| Transcript name | position | ending position |
| T51958_PEA_1_T40 (SEQ ID NO: 1091) | 576 | 972 |

Segment cluster T51958_PEA_{—}1_node_{—}14 (SEQ ID NO:1097) according to the present invention is supported by 27 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088), T51958_PEA_{—}1_T37 (SEQ ID NO:1089), T51958_PEA_{—}1_T39 (SEQ ID NO:1090) and T51958_PEA_{—}1_T41 (SEQ ID NO:1092). Table 21 below describes the starting and ending position of this segment on each transcript.

TABLE 21
Segment location on transcripts

| | Segment | |
|---|---|---|
| | starting | Segment |
| Transcript name | position | ending position |
| T51958_PEA_1_T4 (SEQ ID NO: 1081) | 679 | 869 |
| T51958_PEA_1_T5 (SEQ ID NO: 1082) | 679 | 869 |
| T51958_PEA_1_T6 (SEQ ID NO: 1083) | 679 | 869 |
| T51958_PEA_1_T12 (SEQ ID NO: 1085) | 679 | 869 |
| T51958_PEA_1_T16 (SEQ ID NO: 1086) | 679 | 869 |
| T51958_PEA_1_T33 (SEQ ID NO: 1087) | 679 | 869 |
| T51958_PEA_1_T35 (SEQ ID NO: 1088) | 679 | 869 |
| T51958_PEA_1_T37 (SEQ ID NO: 1089) | 679 | 869 |
| T51958_PEA_1_T39 (SEQ ID NO: 1090) | 679 | 869 |
| T51958_PEA_1_T41 (SEQ ID NO: 1092) | 679 | 869 |

Segment cluster T51958_PEA_{—}1_node_{—}16 (SEQ ID NO:1098) according to the present invention is supported by 25 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088), T51958_PEA_{—}1_T37 (SEQ ID NO:1089), T51958_PEA_{—}1_T39 (SEQ ID NO:1090) and T51958_PEA_{—}1_T41 (SEQ ID NO:1092). Table 22 below describes the starting and ending position of this segment on each transcript.

TABLE 22
Segment location on transcripts

| | Segment | |
|---|---|---|
| | starting | Segment |
| Transcript name | position | ending position |
| T51958_PEA_1_T4 (SEQ ID NO: 1081) | 870 | 1020 |
| T51958_PEA_1_T5 (SEQ ID NO: 1082) | 870 | 1020 |
| T51958_PEA_1_T6 (SEQ ID NO: 1083) | 960 | 1110 |
| T51958_PEA_1_T8 (SEQ ID NO: 1084) | 679 | 829 |
| T51958_PEA_1_T12 (SEQ ID NO: 1085) | 870 | 1020 |
| T51958_PEA_1_T16 (SEQ ID NO: 1086) | 870 | 1020 |
| T51958_PEA_1_T33 (SEQ ID NO: 1087) | 870 | 1020 |
| T51958_PEA_1_T35 (SEQ ID NO: 1088) | 870 | 1020 |
| T51958_PEA_1_T37 (SEQ ID NO: 1089) | 870 | 1020 |
| T51958_PEA_1_T39 (SEQ ID NO: 1090) | 870 | 1020 |
| T51958_PEA_1_T41 (SEQ ID NO: 1092) | 960 | 1110 |

Segment cluster T51958_PEA_{—}1_node_{—}18 (SEQ ID NO:1099) according to the present invention is supported by 26 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088), T51958_PEA_{—}1_T37 (SEQ ID NO:1089), T51958_PEA_{—}1_T39 (SEQ ID NO:1090) and T51958_PEA_{—}1_T41 (SEQ ID NO:1092). Table 23 below describes the starting and ending position of this segment on each transcript.

TABLE 23
Segment location on transcripts

| | Segment | |
|---|---|---|
| | starting | Segment |
| Transcript name | position | ending position |
| T51958_PEA_1_T4 (SEQ ID NO: 1081) | 1021 | 1169 |
| T51958_PEA_1_T5 (SEQ ID NO: 1082) | 1021 | 1169 |
| T51958_PEA_1_T6 (SEQ ID NO: 1083) | 1111 | 1259 |
| T51958_PEA_1_T8 (SEQ ID NO: 1084) | 830 | 978 |
| T51958_PEA_1_T12 (SEQ ID NO: 1085) | 1021 | 1169 |
| T51958_PEA_1_T16 (SEQ ID NO: 1086) | 1021 | 1169 |
| T51958_PEA_1_T33 (SEQ ID NO: 1087) | 1021 | 1169 |
| T51958_PEA_1_T35 (SEQ ID NO: 1088) | 1021 | 1169 |
| T51958_PEA_1_T37 (SEQ ID NO: 1089) | 1021 | 1169 |
| T51958_PEA_1_T39 (SEQ ID NO: 1090) | 1021 | 1169 |
| T51958_PEA_1_T41 (SEQ ID NO: 1092) | 1111 | 1259 |

Segment cluster T51958_PEA_{—}1_node_{—}21 (SEQ ID NO:1100) according to the present invention is supported by 29 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088), T51958_PEA_{—}1_T37 (SEQ ID NO:1089), T51958_PEA_{—}1_T39 (SEQ ID NO:1090) and T51958_PEA_{—}1_T41 (SEQ ID NO:1092). Table 24 below describes the starting and ending position of this segment on each transcript.

TABLE 24
Segment location on transcripts

| | Segment | |
|---|---|---|
| | starting | Segment |
| Transcript name | position | ending position |
| T51958_PEA_1_T4 (SEQ ID NO: 1081) | 1238 | 1436 |
| T51958_PEA_1_T5 (SEQ ID NO: 1082) | 1238 | 1436 |
| T51958_PEA_1_T6 (SEQ ID NO: 1083) | 1328 | 1526 |
| T51958_PEA_1_T8 (SEQ ID NO: 1084) | 1047 | 1245 |
| T51958_PEA_1_T12 (SEQ ID NO: 1085) | 1238 | 1436 |
| T51958_PEA_1_T16 (SEQ ID NO: 1086) | 1238 | 1436 |
| T51958_PEA_1_T33 (SEQ ID NO: 1087) | 1238 | 1436 |
| T51958_PEA_1_T35 (SEQ ID NO: 1088) | 1238 | 1436 |
| T51958_PEA_1_T37 (SEQ ID NO: 1089) | 1238 | 1436 |
| T51958_PEA_1_T39 (SEQ ID NO: 1090) | 1238 | 1436 |
| T51958_PEA_1_T41 (SEQ ID NO: 1092) | 1328 | 1526 |

Segment cluster T51958_PEA_{—}1_node_{—}22 (SEQ ID NO:1101) according to the present invention is supported by 5 libraries. The number of libraries was determined as previously described. This segment can be found in the following transcript(s): T51958_PEA_{—}1_T37 (SEQ ID NO:1089) and T51958_PEA_{—}1_T39 (SEQ ID NO:1090). Table 25 below describes the starting and ending position of this segment on each transcript.

TABLE 25
Segment location on transcripts

| | Segment | |
|---|---|---|
| | starting | Segment |
| Transcript name | position | ending position |
| T51958_PEA_1_T37 (SEQ ID NO: 1089) | 1437 | 2469 |
| T51958_PEA_1_T39 (SEQ ID NO: 1090) | 1437 | 2799 |

Segment cluster T51958_PEA_{—}1_node_{—}24 (SEQ ID NO:1102) according to the present invention is supported by 34 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088) and T51958_PEA_{—}1_T41 (SEQ ID NO:1092). Table 26 below describes the starting and ending position of this segment on each transcript.

TABLE 26
Segment location on transcripts

| | Segment | |
|---|---|---|
| | starting | Segment |
| Transcript name | position | ending position |
| T51958_PEA_1_T4 (SEQ ID NO: 1081) | 1437 | 1570 |
| T51958_PEA_1_T5 (SEQ ID NO: 1082) | 1437 | 1570 |
| T51958_PEA_1_T6 (SEQ ID NO: 1083) | 1527 | 1660 |
| T51958_PEA_1_T8 (SEQ ID NO: 1084) | 1246 | 1379 |
| T51958_PEA_1_T12 (SEQ ID NO: 1085) | 1437 | 1570 |
| T51958_PEA_1_T16 (SEQ ID NO: 1086) | 1437 | 1570 |
| T51958_PEA_1_T33 (SEQ ID NO: 1087) | 1437 | 1570 |
| T51958_PEA_1_T35 (SEQ ID NO: 1088) | 1437 | 1570 |
| T51958_PEA_1_T41 (SEQ ID NO: 1092) | 1527 | 1660 |

Segment cluster T51958_PEA_{—}1_node_{—}27 (SEQ ID NO:1103) according to the present invention is supported by 33 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088) and T51958_PEA_{—}1_T41 (SEQ ID NO:1092). Table 27 below describes the starting and ending position of this segment on each transcript.

TABLE 27
Segment location on transcripts

| | Segment | |
|---|---|---|
| | starting | Segment |
| Transcript name | position | ending position |
| T51958_PEA_1_T4 (SEQ ID NO: 1081) | 1586 | 1706 |
| T51958_PEA_1_T5 (SEQ ID NO: 1082) | 1586 | 1706 |
| T51958_PEA_1_T6 (SEQ ID NO: 1083) | 1676 | 1796 |
| T51958_PEA_1_T8 (SEQ ID NO: 1084) | 1395 | 1515 |
| T51958_PEA_1_T12 (SEQ ID NO: 1085) | 1586 | 1706 |
| T51958_PEA_1_T16 (SEQ ID NO: 1086) | 1586 | 1706 |
| T51958_PEA_1_T33 (SEQ ID NO: 1087) | 1586 | 1706 |
| T51958_PEA_1_T35 (SEQ ID NO: 1088) | 1586 | 1706 |
| T51958_PEA_1_T41 (SEQ ID NO: 1092) | 1676 | 1796 |

Segment cluster T51958_PEA_{—}1_node_{—}29 (SEQ ID NO:1104) according to the present invention is supported by 37 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087) and T51958_PEA_{—}1_T35 (SEQ ID NO:1088). Table 28 below describes the starting and ending position of this segment on each transcript.

TABLE 28
Segment location on transcripts

| | Segment | |
|---|---|---|
| | starting | Segment |
| Transcript name | position | ending position |
| T51958_PEA_1_T4 (SEQ ID NO: 1081) | 1707 | 1826 |
| T51958_PEA_1_T5 (SEQ ID NO: 1082) | 1707 | 1826 |
| T51958_PEA_1_T6 (SEQ ID NO: 1083) | 1797 | 1916 |
| T51958_PEA_1_T8 (SEQ ID NO: 1084) | 1516 | 1635 |
| T51958_PEA_1_T12 (SEQ ID NO: 1085) | 1707 | 1826 |
| T51958_PEA_1_T16 (SEQ ID NO: 1086) | 1707 | 1826 |
| T51958_PEA_1_T33 (SEQ ID NO: 1087) | 1707 | 1826 |
| T51958_PEA_1_T35 (SEQ ID NO: 1088) | 1707 | 1826 |

Segment cluster T51958_PEA_{—}1_node_{—}33 (SEQ ID NO:1105) according to the present invention is supported by 37 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088) and T51958_PEA_{—}1_T41 (SEQ ID NO:1092). Table 29 below describes the starting and ending position of this segment on each transcript.

TABLE 29
Segment location on transcripts

| | Segment | |
|---|---|---|
| | starting | Segment |
| Transcript name | position | ending position |
| T51958_PEA_1_T4 (SEQ ID NO: 1081) | 1827 | 1976 |
| T51958_PEA_1_T5 (SEQ ID NO: 1082) | 1827 | 1976 |
| T51958_PEA_1_T6 (SEQ ID NO: 1083) | 1917 | 2066 |
| T51958_PEA_1_T8 (SEQ ID NO: 1084) | 1636 | 1785 |
| T51958_PEA_1_T12 (SEQ ID NO: 1085) | 1827 | 1976 |
| T51958_PEA_1_T16 (SEQ ID NO: 1086) | 1827 | 1976 |
| T51958_PEA_1_T33 (SEQ ID NO: 1087) | 1827 | 1976 |
| T51958_PEA_1_T35 (SEQ ID NO: 1088) | 1827 | 1976 |
| T51958_PEA_1_T41 (SEQ ID NO: 1092) | 1797 | 1946 |

Segment cluster T51958_PEA_{—}1_node_{—}40 (SEQ ID NO:1106) according to the present invention is supported by 13 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T12 (SEQ ID NO:1085), T51958_PEA_{—}1_T16 (SEQ ID NO:1086), T51958_PEA_{—}1_T33 (SEQ ID NO:1087) and T51958_PEA_{—}1_T35 (SEQ ID NO:1088). Table 30 below describes the starting and ending position of this segment on each transcript.

TABLE 30
Segment location on transcripts

| | Segment | |
|---|---|---|
| | starting | Segment |
| Transcript name | position | ending position |
| T51958_PEA_1_T4 (SEQ ID NO: 1081) | 2256 | 2733 |
| T51958_PEA_1_T5 (SEQ ID NO: 1082) | 2240 | 2717 |
| T51958_PEA_1_T12 (SEQ ID NO: 1085) | 2256 | 2733 |
| T51958_PEA_1_T16 (SEQ ID NO: 1086) | 2256 | 2733 |
| T51958_PEA_1_T33 (SEQ ID NO: 1087) | 2256 | 2733 |
| T51958_PEA_1_T35 (SEQ ID NO: 1088) | 2256 | 2733 |

Segment cluster T51958_PEA_{—}1_node_{—}41 (SEQ ID NO:1107) according to the present invention is supported by 12 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T12 (SEQ ID NO:1085), T51958_PEA_{—}1_T16 (SEQ ID NO:1086) and T51958_PEA_{—}1_T33 (SEQ ID NO:1087). Table 31 below describes the starting and ending position of this segment on each transcript.

TABLE 31
Segment location on transcripts

| | Segment | |
|---|---|---|
| | starting | Segment |
| Transcript name | position | ending position |
| T51958_PEA_1_T4 (SEQ ID NO: 1081) | 2734 | 3372 |
| T51958_PEA_1_T5 (SEQ ID NO: 1082) | 2718 | 3356 |
| T51958_PEA_1_T12 (SEQ ID NO: 1085) | 2734 | 3372 |
| T51958_PEA_1_T16 (SEQ ID NO: 1086) | 2734 | 3372 |
| T51958_PEA_1_T33 (SEQ ID NO: 1087) | 2734 | 3372 |

Segment cluster T51958_PEA_{—}1_node_{—}46 (SEQ ID NO:1108) according to the present invention is supported by 15 libraries. The number of libraries was determined as previously described. This segment can be found in the following transcript(s): T51958_PEA_{—}1_T12 (SEQ ID NO:1085), T51958_PEA_{—}1_T16 (SEQ ID NO:1086) and T51958_PEA_{—}1_T33 (SEQ ID NO:1087). Table 32 below describes the starting and ending position of this segment on each transcript.

TABLE 32
Segment location on transcripts

| | Segment | |
|---|---|---|
| | starting | Segment |
| Transcript name | position | ending position |
| T51958_PEA_1_T12 (SEQ ID NO: 1085) | 3577 | 4406 |
| T51958_PEA_1_T16 (SEQ ID NO: 1086) | 3577 | 4406 |
| T51958_PEA_1_T33 (SEQ ID NO: 1087) | 3577 | 4406 |

Segment cluster T51958_PEA_{—}1_node_{—}51 (SEQ ID NO:1109) according to the present invention is supported by 2 libraries. The number of libraries was determined as previously described. This segment can be found in the following transcript(s): T51958_PEA_{—}1_T33 (SEQ ID NO:1087). Table 33 below describes the starting and ending position of this segment on each transcript.

TABLE 33
Segment location on transcripts

| | Segment | Segment |
|---|---|---|
| | starting | ending |
| Transcript name | position | position |
| T51958_PEA_1_T33 (SEQ ID NO: 1087) | 4563 | 4811 |

Segment cluster T51958_PEA_{—}1_node_{—}55 (SEQ ID NO:1110) according to the present invention is supported by 82 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085) and T51958_PEA_{—}1_T41 (SEQ ID NO:1092). Table 34 below describes the starting and ending position of this segment on each transcript.

TABLE 34
Segment location on transcripts

| | Segment | Segment |
|---|---|---|
| | starting | ending |
| Transcript name | position | position |
| T51958_PEA_1_T4 (SEQ ID NO: 1081) | 3753 | 3965 |
| T51958_PEA_1_T5 (SEQ ID NO: 1082) | 3737 | 3949 |
| T51958_PEA_1_T6 (SEQ ID NO: 1083) | 2726 | 2938 |
| T51958_PEA_1_T8 (SEQ ID NO: 1084) | 2440 | 2652 |
| T51958_PEA_1_T12 (SEQ ID NO: 1085) | 4583 | 4795 |
| T51958_PEA_1_T41 (SEQ ID NO: 1092) | 2606 | 2818 |

Segment cluster T51958_PEA_{—}1_node_{—}67 (SEQ ID NO:1111) according to the present invention is supported by 81 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086) and T51958_PEA_{—}1_T41 (SEQ ID NO:1092). Table 35 below describes the starting and ending position of this segment on each transcript.

TABLE 35
Segment location on transcripts

| | Segment | Segment |
|---|---|---|
| | starting | ending |
| Transcript name | position | position |
| T51958_PEA_1_T4 (SEQ ID NO: 1081) | 4047 | 4198 |
| T51958_PEA_1_T5 (SEQ ID NO: 1082) | 4031 | 4182 |
| T51958_PEA_1_T6 (SEQ ID NO: 1083) | 3020 | 3171 |
| T51958_PEA_1_T8 (SEQ ID NO: 1084) | 2734 | 2885 |
| T51958_PEA_1_T12 (SEQ ID NO: 1085) | 4877 | 5028 |
| T51958_PEA_1_T16 (SEQ ID NO: 1086) | 4644 | 4795 |
| T51958_PEA_1_T41 (SEQ ID NO: 1092) | 2900 | 3051 |

Segment cluster T51958_PEA_{—}1_node_{—}70 (SEQ ID NO:1112) according to the present invention is supported by 85 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086) and T51958_PEA_{—}1_T41 (SEQ ID NO:1092). Table 36 below describes the starting and ending position of this segment on each transcript.

TABLE 36
Segment location on transcripts

| | Segment | Segment |
|---|---|---|
| | starting | ending |
| Transcript name | position | position |
| T51958_PEA_1_T4 (SEQ ID NO: 1081) | 4199 | 4320 |
| T51958_PEA_1_T5 (SEQ ID NO: 1082) | 4183 | 4304 |
| T51958_PEA_1_T6 (SEQ ID NO: 1083) | 3172 | 3293 |
| T51958_PEA_1_T8 (SEQ ID NO: 1084) | 2886 | 3007 |
| T51958_PEA_1_T12 (SEQ ID NO: 1085) | 5029 | 5150 |
| T51958_PEA_1_T16 (SEQ ID NO: 1086) | 4796 | 4917 |
| T51958_PEA_1_T41 (SEQ ID NO: 1092) | 3052 | 3173 |

Segment cluster T51958_PEA_{—}1_node_{—}74 (SEQ ID NO:1113) according to the present invention is supported by 191 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086) and T51958_PEA_{—}1_T41 (SEQ ID NO:1092). Table 37 below describes the starting and ending position of this segment on each transcript.

TABLE 37
Segment location on transcripts

| | Segment | Segment |
|---|---|---|
| | starting | ending |
| Transcript name | position | position |
| T51958_PEA_1_T4 (SEQ ID NO: 1081) | 4378 | 5077 |
| T51958_PEA_1_T5 (SEQ ID NO: 1082) | 4362 | 5061 |
| T51958_PEA_1_T6 (SEQ ID NO: 1083) | 3351 | 4050 |
| T51958_PEA_1_T8 (SEQ ID NO: 1084) | 3065 | 3764 |
| T51958_PEA_1_T12 (SEQ ID NO: 1085) | 5208 | 5907 |
| T51958_PEA_1_T16 (SEQ ID NO: 1086) | 4975 | 5674 |
| T51958_PEA_1_T41 (SEQ ID NO: 1092) | 3231 | 3930 |

Segment cluster T51958_PEA_{—}1_node_{—}78 (SEQ ID NO:1114) according to the present invention is supported by 115 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086) and T51958_PEA_{—}1_T41 (SEQ ID NO:1092). Table 38 below describes the starting and ending position of this segment on each transcript.

TABLE 38
Segment location on transcripts

| | Segment | Segment |
|---|---|---|
| | starting | ending |
| Transcript name | position | position |
| T51958_PEA_1_T4 (SEQ ID NO: 1081) | 5124 | 5376 |
| T51958_PEA_1_T5 (SEQ ID NO: 1082) | 5108 | 5360 |
| T51958_PEA_1_T6 (SEQ ID NO: 1083) | 4097 | 4349 |
| T51958_PEA_1_T8 (SEQ ID NO: 1084) | 3811 | 4063 |
| T51958_PEA_1_T12 (SEQ ID NO: 1085) | 5954 | 6206 |
| T51958_PEA_1_T16 (SEQ ID NO: 1086) | 5721 | 5973 |
| T51958_PEA_1_T41 (SEQ ID NO: 1092) | 3977 | 4229 |

According to an optional embodiment of the present invention, short segments related to the above cluster are also provided. These segments are up to about 120 bp in length, and so are included in a separate description.

Segment cluster T51958_PEA_{—}1_node_{—}11 (SEQ ID NO:11115) according to the present invention is supported by 23 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088), T51958_PEA_{—}1_T37 (SEQ ID NO:1089), T51958_PEA_{—}1_T39 (SEQ ID NO:1090) and T51958_PEA_{—}1_T41 (SEQ ID NO:1092). Table 39 below describes the starting and ending position of this segment on each transcript.

TABLE 39
Segment location on transcripts

| | Segment | Segment |
|---|---|---|
| | starting | ending |
| Transcript name | position | position |
| T51958_PEA_1_T4 (SEQ ID NO: 1081) | 576 | 678 |
| T51958_PEA_1_T5 (SEQ ID NO: 1082) | 576 | 678 |
| T51958_PEA_1_T6 (SEQ ID NO: 1083) | 576 | 678 |
| T51958_PEA_1_T8 (SEQ ID NO: 1084) | 576 | 678 |
| T51958_PEA_1_T12 (SEQ ID NO: 1085) | 576 | 678 |
| T51958_PEA_1_T16 (SEQ ID NO: 1086) | 576 | 678 |
| T51958_PEA_1_T33 (SEQ ID NO: 1087) | 576 | 678 |
| T51958_PEA_1_T35 (SEQ ID NO: 1088) | 576 | 678 |
| T51958_PEA_1_T37 (SEQ ID NO: 1089) | 576 | 678 |
| T51958_PEA_1_T39 (SEQ ID NO: 1090) | 576 | 678 |
| T51958_PEA_1_T41 (SEQ ID NO: 1092) | 576 | 678 |

Segment cluster T51958_PEA_{—}1_node_{—}15 (SEQ ID NO:1116) according to the present invention is supported by 2 libraries. The number of libraries was determined as previously described. This segment can be found in the following transcript(s): T51958_PEA_{—}1_T6 (SEQ ID NO:1083) and T51958_PEA_{—}1_T41 (SEQ ID NO:1092). Table 40 below describes the starting and ending position of this segment on each transcript.

TABLE 40
Segment location on transcripts

| | Segment | Segment |
|---|---|---|
| | starting | ending |
| Transcript name | position | position |
| T51958_PEA_1_T6 (SEQ ID NO: 1083) | 870 | 959 |
| T51958_PEA_1_T41 (SEQ ID NO: 1092) | 870 | 959 |

Segment cluster T51958_PEA_{—}1_node_{—}20 (SEQ ID NO:1117) according to the present invention is supported by 25 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088), T51958_PEA_{—}1_T37 (SEQ ID NO:1089), T51958_PEA_{—}1_T39 (SEQ ID NO:1090) and T51958_PEA_{—}1_T41 (SEQ ID NO:1092). Table 41 below describes the starting and ending position of this segment on each transcript.

TABLE 41
Segment location on transcripts

| | Segment | Segment |
|---|---|---|
| | starting | ending |
| Transcript name | position | position |
| T51958_PEA_1_T4 (SEQ ID NO: 1081) | 1170 | 1237 |
| T51958_PEA_1_T5 (SEQ ID NO: 1082) | 1170 | 1237 |
| T51958_PEA_1_T6 (SEQ ID NO: 1083) | 1260 | 1327 |
| T51958_PEA_1_T8 (SEQ ID NO: 1084) | 979 | 1046 |
| T51958_PEA_1_T12 (SEQ ID NO: 1085) | 1170 | 1237 |
| T51958_PEA_1_T16 (SEQ ID NO: 1086) | 1170 | 1237 |
| T51958_PEA_1_T33 (SEQ ID NO: 1087) | 1170 | 1237 |
| T51958_PEA_1_T35 (SEQ ID NO: 1088) | 1170 | 1237 |
| T51958_PEA_1_T37 (SEQ ID NO: 1089) | 1170 | 1237 |
| T51958_PEA_1_T39 (SEQ ID NO: 1090) | 1170 | 1237 |
| T51958_PEA_1_T41 (SEQ ID NO: 1092) | 1260 | 1327 |

Segment cluster T51958_PEA_{—}1_node_{—}26 (SEQ ID NO:1118) according to the present invention can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088) and T51958_PEA_{—}1_T41 (SEQ ID NO:1092). Table 42 below describes the starting and ending position of this segment on each transcript.

TABLE 42
Segment location on transcripts

| | Segment | Segment |
|---|---|---|
| | starting | ending |
| Transcript name | position | position |
| T51958_PEA_1_T4 (SEQ ID NO: 1081) | 1571 | 1585 |
| T51958_PEA_1_T5 (SEQ ID NO: 1082) | 1571 | 1585 |
| T51958_PEA_1_T6 (SEQ ID NO: 1083) | 1661 | 1675 |
| T51958_PEA_1_T8 (SEQ ID NO: 1084) | 1380 | 1394 |
| T51958_PEA_1_T12 (SEQ ID NO: 1085) | 1571 | 1585 |
| T51958_PEA_1_T16 (SEQ ID NO: 1086) | 1571 | 1585 |
| T51958_PEA_1_T33 (SEQ ID NO: 1087) | 1571 | 1585 |
| T51958_PEA_1_T35 (SEQ ID NO: 1088) | 1571 | 1585 |
| T51958_PEA_1_T41 (SEQ ID NO: 1092) | 1661 | 1675 |

Segment cluster T51958_PEA_{—}1_node_{—}35 (SEQ ID NO:1119) according to the present invention is supported by 41 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088) and T51958_PEA_{—}1_T41 (SEQ ID NO:1092). Table 43 below describes the starting and ending position of this segment on each transcript.

TABLE 43
Segment location on transcripts

| | Segment | Segment |
|---|---|---|
| | starting | ending |
| Transcript name | position | position |
| T51958_PEA_1_T4 (SEQ ID NO: 1081) | 1977 | 2087 |
| T51958_PEA_1_T5 (SEQ ID NO: 1082) | 1977 | 2087 |
| T51958_PEA_1_T6 (SEQ ID NO: 1083) | 2067 | 2177 |
| T51958_PEA_1_T8 (SEQ ID NO: 1084) | 1786 | 1896 |
| T51958_PEA_1_T12 (SEQ ID NO: 1085) | 1977 | 2087 |
| T51958_PEA_1_T16 (SEQ ID NO: 1086) | 1977 | 2087 |
| T51958_PEA_1_T33 (SEQ ID NO: 1087) | 1977 | 2087 |
| T51958_PEA_1_T35 (SEQ ID NO: 1088) | 1977 | 2087 |
| T51958_PEA_1_T41 (SEQ ID NO: 1092) | 1947 | 2057 |

Segment cluster T51958_PEA_{—}1_node_{—}36 (SEQ ID NO:1120) according to the present invention is supported by 35 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088) and T51958_PEA_{—}1_T41 (SEQ ID NO:1092). Table 44 below describes the starting and ending position of this segment on each transcript.

TABLE 44
Segment location on transcripts

| | Segment | Segment |
|---|---|---|
| | starting | ending |
| Transcript name | position | position |
| T51958_PEA_1_T4 (SEQ ID NO: 1081) | 2088 | 2127 |
| T51958_PEA_1_T5 (SEQ ID NO: 1082) | 2088 | 2127 |
| T51958_PEA_1_T6 (SEQ ID NO: 1083) | 2178 | 2217 |
| T51958_PEA_1_T8 (SEQ ID NO: 1084) | 1897 | 1936 |
| T51958_PEA_1_T12 (SEQ ID NO: 1085) | 2088 | 2127 |
| T51958_PEA_1_T16 (SEQ ID NO: 1086) | 2088 | 2127 |
| T51958_PEA_1_T33 (SEQ ID NO: 1087) | 2088 | 2127 |
| T51958_PEA_1_T35 (SEQ ID NO: 1088) | 2088 | 2127 |
| T51958_PEA_1_T41 (SEQ ID NO: 1092) | 2058 | 2097 |

Segment cluster T51958_PEA_{—}1_node_{—}38 (SEQ ID NO:1121) according to the present invention can be found in the following transcript(s): T51958_PEA_{—}1_T4 (SEQ ID NO:1081), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088) and T51958_PEA_{—}1_T41 (SEQ ID NO:1092). Table 45 below describes the starting and ending position of this segment on each transcript.

TABLE 45
Segment location on transcripts

| | Segment | Segment |
|---|---|---|
| | starting | ending |
| Transcript name | position | position |
| T51958_PEA_1_T4 (SEQ ID NO: 1081) | 2128 | 2143 |
| T51958_PEA_1_T6 (SEQ ID NO: 1083) | 2218 | 2233 |
| T51958_PEA_1_T8 (SEQ ID NO: 1084) | 1937 | 1952 |
| T51958_PEA_1_T12 (SEQ ID NO: 1085) | 2128 | 2143 |
| T51958_PEA_1_T16 (SEQ ID NO: 1086) | 2128 | 2143 |
| T51958_PEA_1_T33 (SEQ ID NO: 1087) | 2128 | 2143 |
| T51958_PEA_1_T35 (SEQ ID NO: 1088) | 2128 | 2143 |
| T51958_PEA_1_T41 (SEQ ID NO: 1092) | 2098 | 2113 |

Segment cluster T51958_PEA_{—}1_node_{—}39 (SEQ ID NO:1122) according to the present invention is supported by 40 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087), T51958_PEA_{—}1_T35 (SEQ ID NO:1088) and T51958_PEA_{—}1_T41 (SEQ ID NO:1092). Table 46 below describes the starting and ending position of this segment on each transcript.

TABLE 46
Segment location on transcripts

| | Segment | Segment |
|---|---|---|
| | starting | ending |
| Transcript name | position | position |
| T51958_PEA_1_T4 (SEQ ID NO: 1081) | 2144 | 2255 |
| T51958_PEA_1_T5 (SEQ ID NO: 1082) | 2128 | 2239 |
| T51958_PEA_1_T6 (SEQ ID NO: 1083) | 2234 | 2345 |
| T51958_PEA_1_T8 (SEQ ID NO: 1084) | 1953 | 2064 |
| T51958_PEA_1_T12 (SEQ ID NO: 1085) | 2144 | 2255 |
| T51958_PEA_1_T16 (SEQ ID NO: 1086) | 2144 | 2255 |
| T51958_PEA_1_T33 (SEQ ID NO: 1087) | 2144 | 2255 |
| T51958_PEA_1_T35 (SEQ ID NO: 1088) | 2144 | 2255 |
| T51958_PEA_1_T41 (SEQ ID NO: 1092) | 2114 | 2225 |

Segment cluster T51958_PEA_{—}1_node_{—}42 (SEQ ID NO:1123) according to the present invention can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T12 (SEQ ID NO:1085), T51958_PEA_{—}1_T16 (SEQ ID NO:1086), T51958_PEA_{—}1_T33 (SEQ ID NO:1087) and T51958_PEA_{—}1_T41 (SEQ ID NO:1092). Table 47 below describes the starting and ending position of this segment on each transcript.

TABLE 47
Segment location on transcripts

| | Segment | Segment |
|---|---|---|
| | starting | ending |
| Transcript name | position | position |
| T51958_PEA_1_T4 (SEQ ID NO: 1081) | 3373 | 3377 |
| T51958_PEA_1_T5 (SEQ ID NO: 1082) | 3357 | 3361 |
| T51958_PEA_1_T6 (SEQ ID NO: 1083) | 2346 | 2350 |
| T51958_PEA_1_T12 (SEQ ID NO: 1085) | 3373 | 3377 |
| T51958_PEA_1_T16 (SEQ ID NO: 1086) | 3373 | 3377 |
| T51958_PEA_1_T33 (SEQ ID NO: 1087) | 3373 | 3377 |
| T51958_PEA_1_T41 (SEQ ID NO: 1092) | 2226 | 2230 |

Segment cluster T51958_PEA_{—}1_node_{—}43 (SEQ ID NO:1124) according to the present invention is supported by 50 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087) and T51958_PEA_{—}1_T41 (SEQ ID NO:1092). Table 48 below describes the starting and ending position of this segment on each transcript.

TABLE 48
Segment location on transcripts

| | Segment | Segment |
|---|---|---|
| | starting | ending |
| Transcript name | position | position |
| T51958_PEA_1_T4 (SEQ ID NO: 1081) | 3378 | 3496 |
| T51958_PEA_1_T5 (SEQ ID NO: 1082) | 3362 | 3480 |
| T51958_PEA_1_T6 (SEQ ID NO: 1083) | 2351 | 2469 |
| T51958_PEA_1_T8 (SEQ ID NO: 1084) | 2065 | 2183 |
| T51958_PEA_1_T12 (SEQ ID NO: 1085) | 3378 | 3496 |
| T51958_PEA_1_T16 (SEQ ID NO: 1086) | 3378 | 3496 |
| T51958_PEA_1_T33 (SEQ ID NO: 1087) | 3378 | 3496 |
| T51958_PEA_1_T41 (SEQ ID NO: 1092) | 2231 | 2349 |

Segment cluster T51958_PEA_{—}1_node_{—}44 (SEQ ID NO:1125) according to the present invention is supported by 57 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087) and T51958_PEA_{—}1_T41 (SEQ ID NO:1092). Table 49 below describes the starting and ending position of this segment on each transcript.

TABLE 49
Segment location on transcripts

| | Segment | Segment |
|---|---|---|
| | starting | ending |
| Transcript name | position | position |
| T51958_PEA_1_T4 (SEQ ID NO: 1081) | 3497 | 3560 |
| T51958_PEA_1_T5 (SEQ ID NO: 1082) | 3481 | 3544 |
| T51958_PEA_1_T6 (SEQ ID NO: 1083) | 2470 | 2533 |
| T51958_PEA_1_T8 (SEQ ID NO: 1084) | 2184 | 2247 |
| T51958_PEA_1_T12 (SEQ ID NO: 1085) | 3497 | 3560 |
| T51958_PEA_1_T16 (SEQ ID NO: 1086) | 3497 | 3560 |
| T51958_PEA_1_T33 (SEQ ID NO: 1087) | 3497 | 3560 |
| T51958_PEA_1_T41 (SEQ ID NO: 1092) | 2350 | 2413 |

Segment cluster T51958_PEA_{—}1_node_{—}45 (SEQ ID NO:1126) according to the present invention can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087) and T51958_PEA_{—}1_T41 (SEQ ID NO:1092). Table 50 below describes the starting and ending position of this segment on each transcript.

TABLE 50
Segment location on transcripts

| | Segment | Segment |
|---|---|---|
| | starting | ending |
| Transcript name | position | position |
| T51958_PEA_1_T4 (SEQ ID NO: 1081) | 3561 | 3576 |
| T51958_PEA_1_T5 (SEQ ID NO: 1082) | 3545 | 3560 |
| T51958_PEA_1_T6 (SEQ ID NO: 1083) | 2534 | 2549 |
| T51958_PEA_1_T8 (SEQ ID NO: 1084) | 2248 | 2263 |
| T51958_PEA_1_T12 (SEQ ID NO: 1085) | 3561 | 3576 |
| T51958_PEA_1_T16 (SEQ ID NO: 1086) | 3561 | 3576 |
| T51958_PEA_1_T33 (SEQ ID NO: 1087) | 3561 | 3576 |
| T51958_PEA_1_T41 (SEQ ID NO: 1092) | 2414 | 2429 |

Segment cluster T51958_PEA_{—}1_node_{—}47 (SEQ ID NO:1127) according to the present invention is supported by 65 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087) and T51958_PEA_{—}1_T41 (SEQ ID NO:1092). Table 51 below describes the starting and ending position of this segment on each transcript.

TABLE 51
Segment location on transcripts

| | Segment | Segment |
|---|---|---|
| | starting | ending |
| Transcript name | position | position |
| T51958_PEA_1_T4 (SEQ ID NO: 1081) | 3577 | 3651 |
| T51958_PEA_1_T5 (SEQ ID NO: 1082) | 3561 | 3635 |
| T51958_PEA_1_T6 (SEQ ID NO: 1083) | 2550 | 2624 |
| T51958_PEA_1_T8 (SEQ ID NO: 1084) | 2264 | 2338 |
| T51958_PEA_1_T12 (SEQ ID NO: 1085) | 4407 | 4481 |
| T51958_PEA_1_T16 (SEQ ID NO: 1086) | 4407 | 4481 |
| T51958_PEA_1_T33 (SEQ ID NO: 1087) | 4407 | 4481 |
| T51958_PEA_1_T41 (SEQ ID NO: 1092) | 2430 | 2504 |

Segment cluster T51958_PEA_{—}1_node_{—}48 (SEQ ID NO:1128) according to the present invention is supported by 68 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087) and T51958_PEA_{—}1_T41 (SEQ ID NO:1092). Table 52 below describes the starting and ending position of this segment on each transcript.

TABLE 52
Segment location on transcripts

| | Segment | Segment |
|---|---|---|
| | starting | ending |
| Transcript name | position | position |
| T51958_PEA_1_T4 (SEQ ID NO: 1081) | 3652 | 3681 |
| T51958_PEA_1_T5 (SEQ ID NO: 1082) | 3636 | 3665 |
| T51958_PEA_1_T6 (SEQ ID NO: 1083) | 2625 | 2654 |
| T51958_PEA_1_T8 (SEQ ID NO: 1084) | 2339 | 2368 |
| T51958_PEA_1_T12 (SEQ ID NO: 1085) | 4482 | 4511 |
| T51958_PEA_1_T16 (SEQ ID NO: 1086) | 4482 | 4511 |
| T51958_PEA_1_T33 (SEQ ID NO: 1087) | 4482 | 4511 |
| T51958_PEA_1_T41 (SEQ ID NO: 1092) | 2505 | 2534 |

Segment cluster T51958_PEA_{—}1_node_{—}49 (SEQ ID NO:1129) according to the present invention is supported by 70 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087) and T51958_PEA_{—}1_T41 (SEQ ID NO:1092). Table 53 below describes the starting and ending position of this segment on each transcript.

TABLE 53
Segment location on transcripts

| | Segment | Segment |
|---|---|---|
| | starting | ending |
| Transcript name | position | position |
| T51958_PEA_1_T4 (SEQ ID NO: 1081) | 3682 | 3717 |
| T51958_PEA_1_T5 (SEQ ID NO: 1082) | 3666 | 3701 |
| T51958_PEA_1_T6 (SEQ ID NO: 1083) | 2655 | 2690 |
| T51958_PEA_1_T8 (SEQ ID NO: 1084) | 2369 | 2404 |
| T51958_PEA_1_T12 (SEQ ID NO: 1085) | 4512 | 4547 |
| T51958_PEA_1_T16 (SEQ ID NO: 1086) | 4512 | 4547 |
| T51958_PEA_1_T33 (SEQ ID NO: 1087) | 4512 | 4547 |
| T51958_PEA_1_T41 (SEQ ID NO: 1092) | 2535 | 2570 |

Segment cluster T51958_PEA_{—}1_node_{—}50 (SEQ ID NO:1130) according to the present invention can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086), T51958_PEA_{—}1_T33 (SEQ ID NO:1087) and T51958_PEA_{—}1_T41 (SEQ ID NO:1092). Table 54 below describes the starting and ending position of this segment on each transcript.

TABLE 54
Segment location on transcripts

| | Segment | Segment |
|---|---|---|
| | starting | ending |
| Transcript name | position | position |
| T51958_PEA_1_T4 (SEQ ID NO: 1081) | 3718 | 3732 |
| T51958_PEA_1_T5 (SEQ ID NO: 1082) | 3702 | 3716 |
| T51958_PEA_1_T6 (SEQ ID NO: 1083) | 2691 | 2705 |
| T51958_PEA_1_T8 (SEQ ID NO: 1084) | 2405 | 2419 |
| T51958_PEA_1_T12 (SEQ ID NO: 1085) | 4548 | 4562 |
| T51958_PEA_1_T16 (SEQ ID NO: 1086) | 4548 | 4562 |
| T51958_PEA_1_T33 (SEQ ID NO: 1087) | 4548 | 4562 |
| T51958_PEA_1_T41 (SEQ ID NO: 1092) | 2571 | 2585 |

Segment cluster T51958_PEA_{—}1_node_{—}54 (SEQ ID NO:1131) according to the present invention can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085) and T51958_PEA_{—}1_T41 (SEQ ID NO:1092). Table 55 below describes the starting and ending position of this segment on each transcript.

TABLE 55
Segment location on transcripts

| | Segment | Segment |
|---|---|---|
| | starting | ending |
| Transcript name | position | position |
| T51958_PEA_1_T4 (SEQ ID NO: 1081) | 3733 | 3752 |
| T51958_PEA_1_T5 (SEQ ID NO: 1082) | 3717 | 3736 |
| T51958_PEA_1_T6 (SEQ ID NO: 1083) | 2706 | 2725 |
| T51958_PEA_1_T8 (SEQ ID NO: 1084) | 2420 | 2439 |
| T51958_PEA_1_T12 (SEQ ID NO: 1085) | 4563 | 4582 |
| T51958_PEA_1_T41 (SEQ ID NO: 1092) | 2586 | 2605 |

Segment cluster T51958_PEA_{—}1_node_{—}61 (SEQ ID NO:1132) according to the present invention is supported by 72 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086) and T51958_PEA_{—}1_T41 (SEQ ID NO:1092). Table 56 below describes the starting and ending position of this segment on each transcript.

TABLE 56
Segment location on transcripts

| | Segment | Segment |
|---|---|---|
| | starting | ending |
| Transcript name | position | position |
| T51958_PEA_1_T4 (SEQ ID NO: 1081) | 3966 | 4046 |
| T51958_PEA_1_T5 (SEQ ID NO: 1082) | 3950 | 4030 |
| T51958_PEA_1_T6 (SEQ ID NO: 1083) | 2939 | 3019 |
| T51958_PEA_1_T8 (SEQ ID NO: 1084) | 2653 | 2733 |
| T51958_PEA_1_T12 (SEQ ID NO: 1085) | 4796 | 4876 |
| T51958_PEA_1_T16 (SEQ ID NO: 1086) | 4563 | 4643 |
| T51958_PEA_1_T41 (SEQ ID NO: 1092) | 2819 | 2899 |

Segment cluster T51958_PEA_{—}1_node_{—}71 (SEQ ID NO:1133) according to the present invention is supported by 80 libraries. The number of libraries was determined as previously described. This segment can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086) and T51958_PEA_{—}1_T41 (SEQ ID NO:1092). Table 57 below describes the starting and ending position of this segment on each transcript.

TABLE 57
Segment location on transcripts

| | Segment | Segment |
|---|---|---|
| | starting | ending |
| Transcript name | position | position |
| T51958_PEA_1_T4 (SEQ ID NO: 1081) | 4321 | 4362 |
| T51958_PEA_1_T5 (SEQ ID NO: 1082) | 4305 | 4346 |
| T51958_PEA_1_T6 (SEQ ID NO: 1083) | 3294 | 3335 |
| T51958_PEA_1_T8 (SEQ ID NO: 1084) | 3008 | 3049 |
| T51958_PEA_1_T12 (SEQ ID NO: 1085) | 5151 | 5192 |
| T51958_PEA_1_T16 (SEQ ID NO: 1086) | 4918 | 4959 |
| T51958_PEA_1_T41 (SEQ ID NO: 1092) | 3174 | 3215 |

Segment cluster T51958_PEA_{—}1_node_{—}72 (SEQ ID NO:1134) according to the present invention can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086) and T51958_PEA_{—}1_T41 (SEQ ID NO:1092). Table 58 below describes the starting and ending position of this segment on each transcript.

TABLE 58
Segment location on transcripts

| | Segment | Segment |
|---|---|---|
| | starting | ending |
| Transcript name | position | position |
| T51958_PEA_1_T4 (SEQ ID NO: 1081) | 4363 | 4377 |
| T51958_PEA_1_T5 (SEQ ID NO: 1082) | 4347 | 4361 |
| T51958_PEA_1_T6 (SEQ ID NO: 1083) | 3336 | 3350 |
| T51958_PEA_1_T8 (SEQ ID NO: 1084) | 3050 | 3064 |
| T51958_PEA_1_T12 (SEQ ID NO: 1085) | 5193 | 5207 |
| T51958_PEA_1_T16 (SEQ ID NO: 1086) | 4960 | 4974 |
| T51958_PEA_1_T41 (SEQ ID NO: 1092) | 3216 | 3230 |

Segment cluster T51958_PEA_{—}1_node_{—}75 (SEQ ID NO:1135) according to the present invention can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086) and T51958_PEA_{—}1_T41 (SEQ ID NO:1092). Table 59 below describes the starting and ending position of this segment on each transcript.

TABLE 59
Segment location on transcripts

| | Segment | Segment |
|---|---|---|
| | starting | ending |
| Transcript name | position | position |
| T51958_PEA_1_T4 (SEQ ID NO: 1081) | 5078 | 5084 |
| T51958_PEA_1_T5 (SEQ ID NO: 1082) | 5062 | 5068 |
| T51958_PEA_1_T6 (SEQ ID NO: 1083) | 4051 | 4057 |
| T51958_PEA_1_T8 (SEQ ID NO: 1084) | 3765 | 3771 |
| T51958_PEA_1_T12 (SEQ ID NO: 1085) | 5908 | 5914 |
| T51958_PEA_1_T16 (SEQ ID NO: 1086) | 5675 | 5681 |
| T51958_PEA_1_T41 (SEQ ID NO: 1092) | 3931 | 3937 |

Segment cluster T51958_PEA_{—}1_node_{—}76 (SEQ ID NO:1136) according to the present invention can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084), T51958_PEA_{—}1_T12 (SEQ ID NO:1085), T51958_PEA_{—}1_T16 (SEQ ID NO:1086) and T51958_PEA_{—}1_T41 (SEQ ID NO:1092). Table 60 below describes the starting and ending position of this segment on each transcript.

TABLE 60
Segment location on transcripts

| | Segment | Segment |
|---|---|---|
| | starting | ending |
| Transcript name | position | position |
| T51958_PEA_1_T4 (SEQ ID NO: 1081) | 5085 | 5107 |
| T51958_PEA_1_T5 (SEQ ID NO: 1082) | 5069 | 5091 |
| T51958_PEA_1_T6 (SEQ ID NO: 1083) | 4058 | 4080 |
| T51958_PEA_1_T8 (SEQ ID NO: 1084) | 3772 | 3794 |
| T51958_PEA_1_T12 (SEQ ID NO: 1085) | 5915 | 5937 |
| T51958_PEA_1_T16 (SEQ ID NO: 1086) | 5682 | 5704 |
| T51958_PEA_1_T41 (SEQ ID NO: 1092) | 3938 | 3960 |

Segment cluster T51958_PEA_{—}1_node_{—}77 (SEQ ID NO:1137) according to the present invention can be found in the following transcript(s): T51958_PEA_{—}1_T4 (SEQ ID NO:1081), T51958_PEA_{—}1_T5 (SEQ ID NO:1082), T51958_PEA_{—}1_T6 (SEQ ID NO:1083), T51958_PEA_{—}1_T8 (SEQ ID NO:1084),

T51958_PEA_{—}1_T12 (SEQ ID NO:1085), T51958_PEA_{—}1_T16 (SEQ ID NO:1086) and T51958_PEA_{—}1_T41 (SEQ ID NO:1092). Table 61 below describes the starting and ending position of this segment on each transcript.

TABLE 61
Segment location on transcripts

| | Segment | Segment |
|---|---|---|
| | starting | ending |
| Transcript name | position | position |
| T51958_PEA_1_T4 (SEQ ID NO: 1081) | 5108 | 5123 |
| T51958_PEA_1_T5 (SEQ ID NO: 1082) | 5092 | 5107 |
| T51958_PEA_1_T6 (SEQ ID NO: 1083) | 4081 | 4096 |
| T51958_PEA_1_T8 (SEQ ID NO: 1084) | 3795 | 3810 |
| T51958_PEA_1_T12 (SEQ ID NO: 1085) | 5938 | 5953 |
| T51958_PEA_1_T16 (SEQ ID NO: 1086) | 5705 | 5720 |
| T51958_PEA_1_T41 (SEQ ID NO: 1092) | 3961 | 3976 |

Segment cluster T51958_PEA_{—}1_node_{—}80 (SEQ ID NO:1138) according to the present invention is supported by 1 libraries. The number of libraries was determined as previously described. This segment can be found in the following transcript(s): T51958_PEA_{—}1_T35 (SEQ ID NO:1088). Table 62 below describes the starting and ending position of this segment on each transcript.

TABLE 62
Segment location on transcripts

| | Segment | Segment |
|---|---|---|
| | starting | ending |
| Transcript name | position | position |
| T51958_PEA_1_T35 (SEQ ID NO: 1088) | 2734 | 2788 |

Segment cluster T51958_PEA_{—}1_node_{—}82 (SEQ ID NO:1139) according to the present invention is supported by 1 libraries. The number of libraries was determined as previously described. This segment can be found in the following transcript(s): T51958_PEA_{—}1_T35 (SEQ ID NO:1088). Table 63 below describes the starting and ending position of this segment on each transcript.

TABLE 63
Segment location on transcripts

| | Segment | Segment |
|---|---|---|
| Transcript name | starting position | ending position |
| T51958_PEA_1_T35 | 2789 | 2877 |
| (SEQ ID NO: 1088) | | |

Segment cluster T51958_PEA_{—}1_node_{—}84 (SEQ ID NO:1140) according to the present invention is supported by 1 libraries. The number of libraries was determined as previously described. This segment can be found in the following transcript(s): T51958_PEA_{—}1_T35 (SEQ ID NO:1088). Table 64 below describes the starting and ending position of this segment on each transcript.

TABLE 64
Segment location on transcripts

| | Segment | Segment |
|---|---|---|
| Transcript name | starting position | ending position |
| T51958_PEA_1_T35 | 2878 | 2946 |
| (SEQ ID NO: 1088) | | |

### Variant Protein Alignment to the Previously Known Protein:

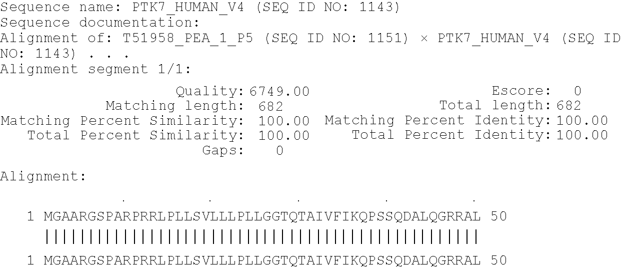

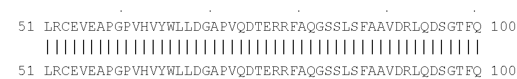

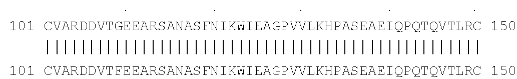

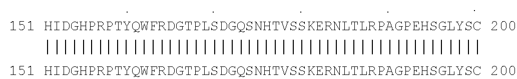

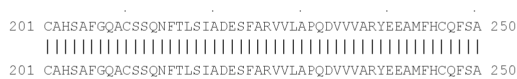

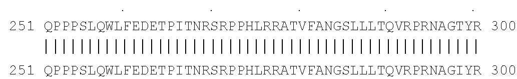

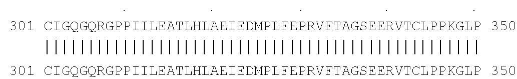

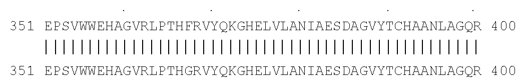

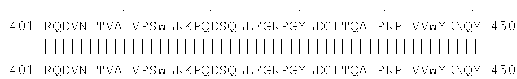

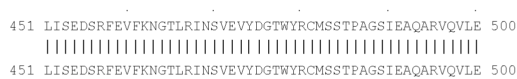

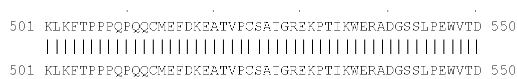

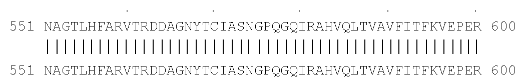

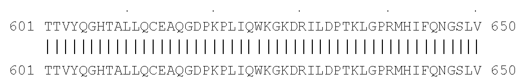

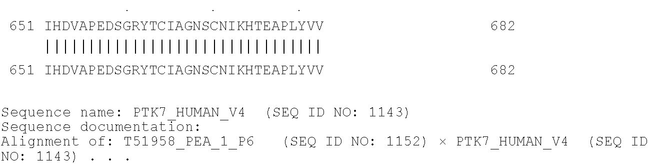

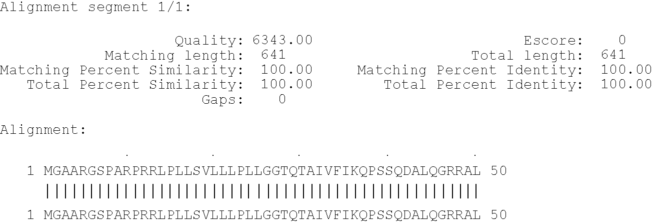

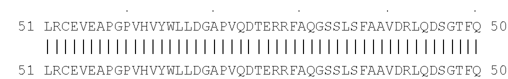

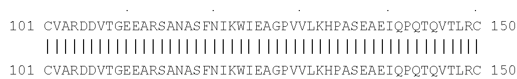

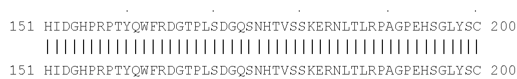

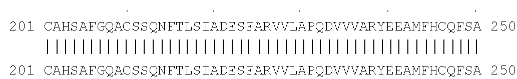

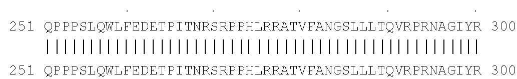

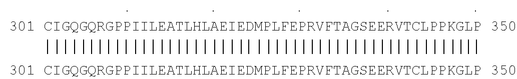

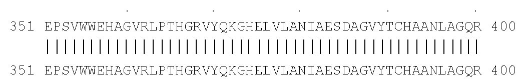

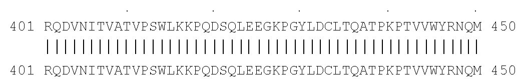

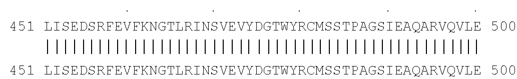

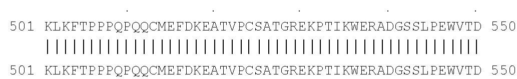

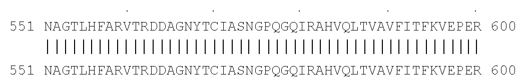

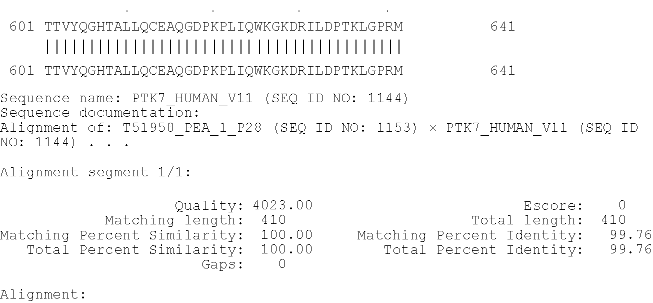

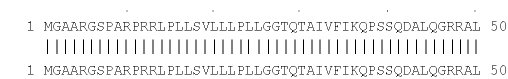

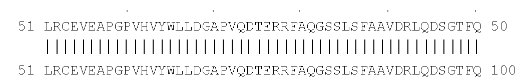

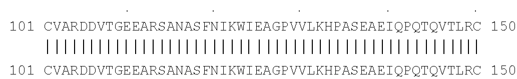

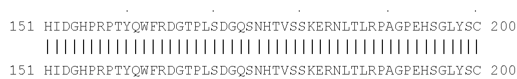

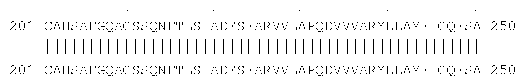

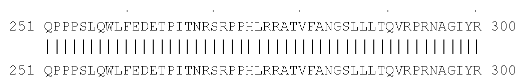

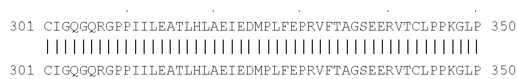

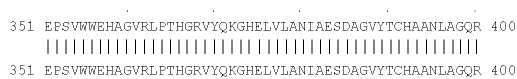

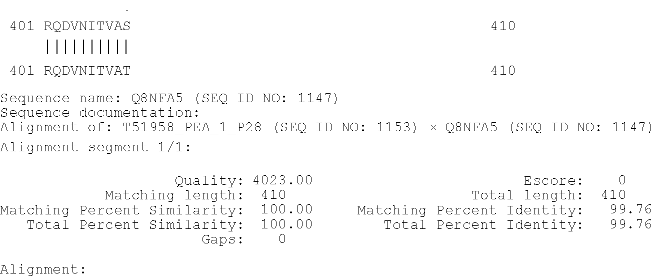

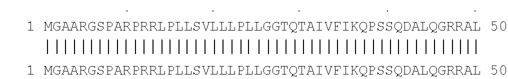

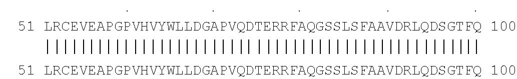

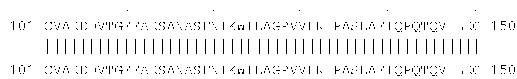

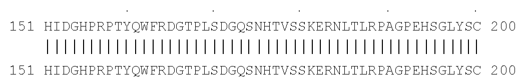

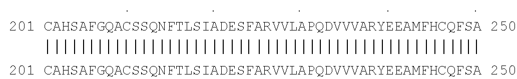

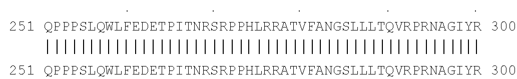

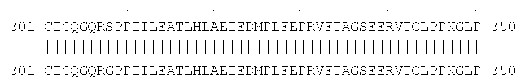

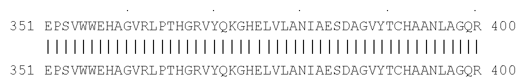

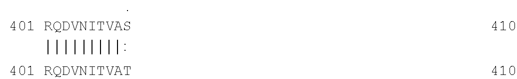

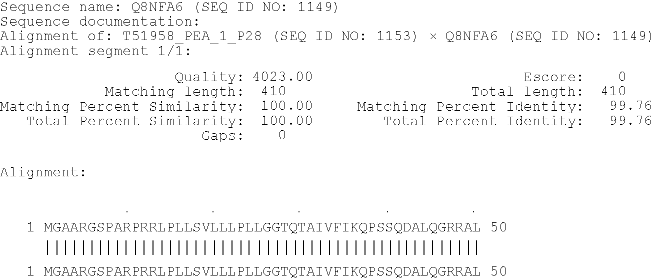

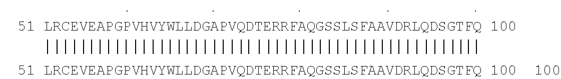

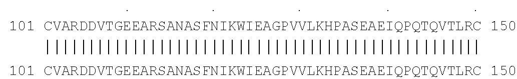

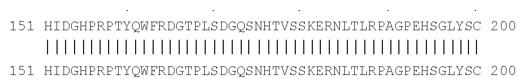

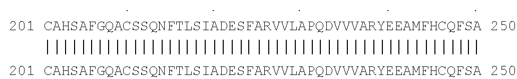

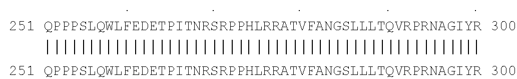

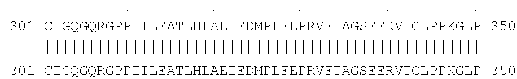

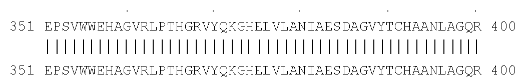

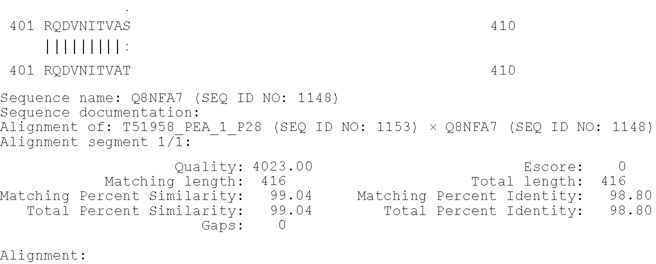

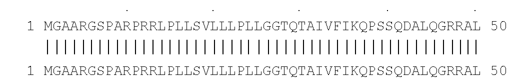

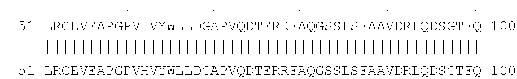

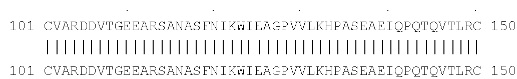

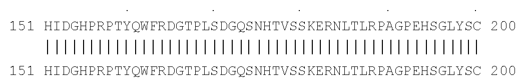

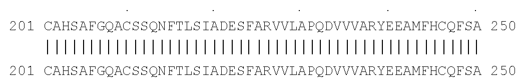

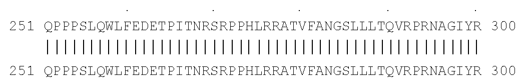

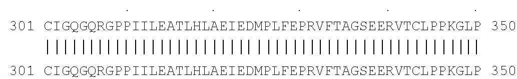

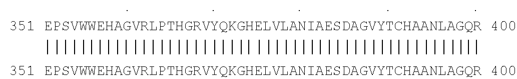

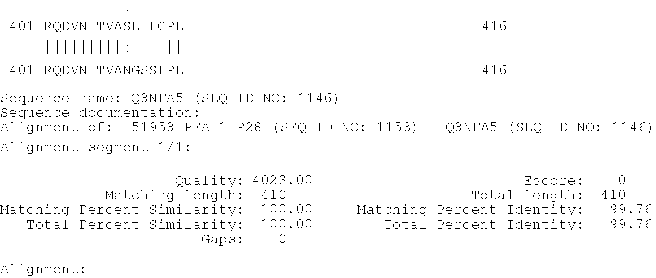

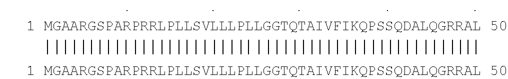

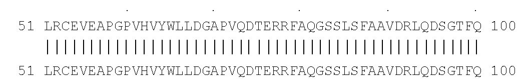

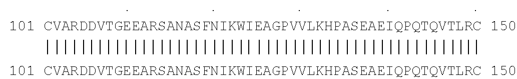

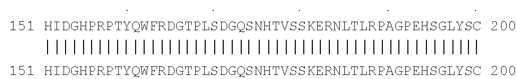

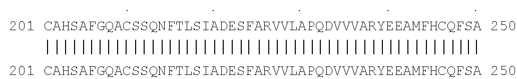

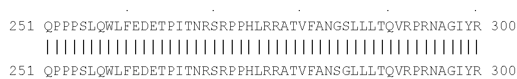

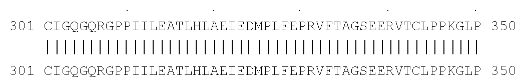

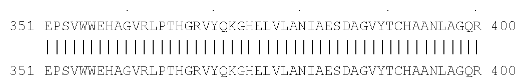

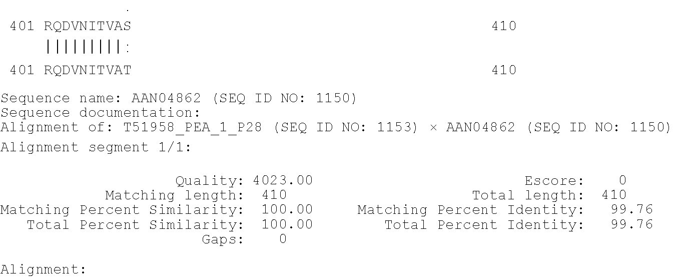

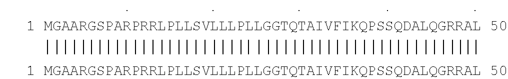

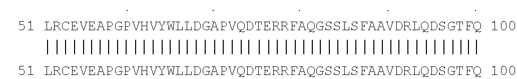

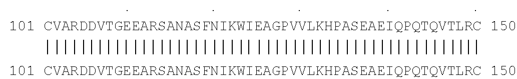

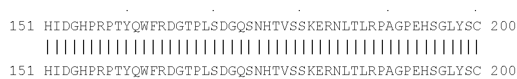

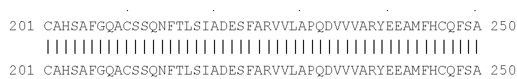

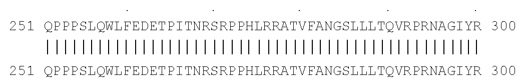

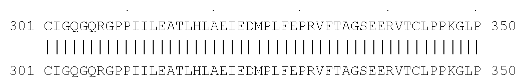

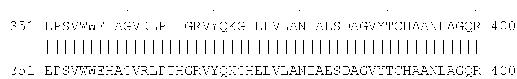

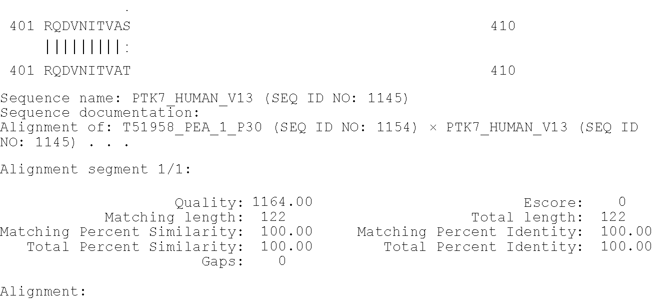

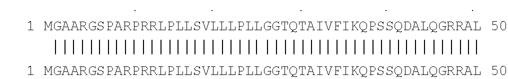

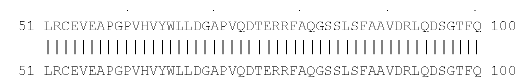

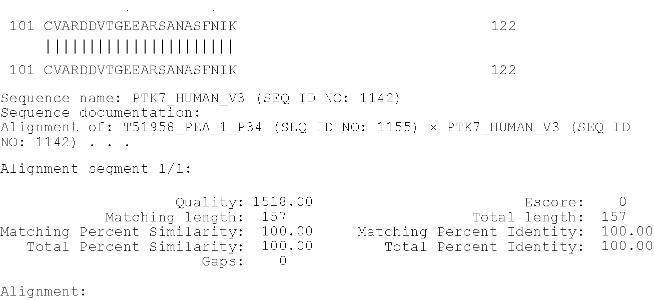

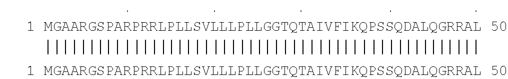

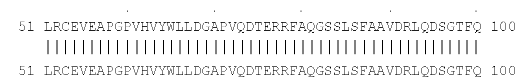

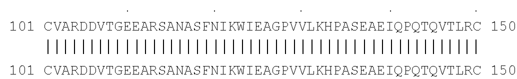

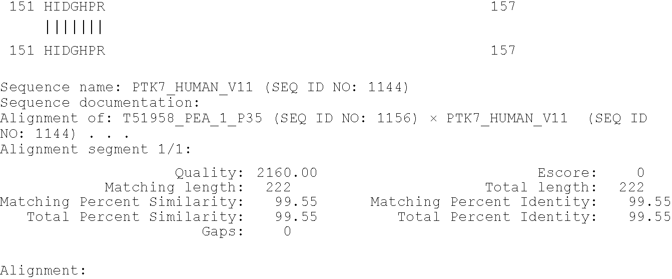

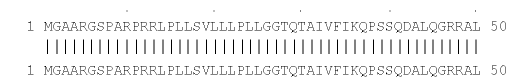

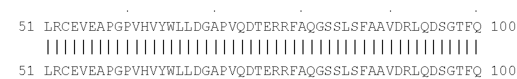

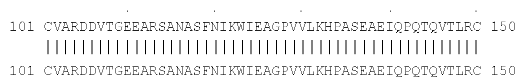

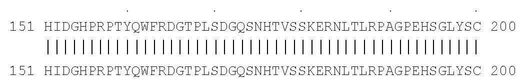

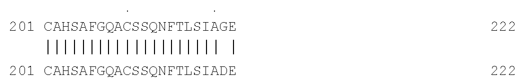

### Expression of Homo sapiens PTK7 Protein Tyrosine Kinase 7 (PTK7) T51958 Transcripts which are Detectable by Amplicon as Depicted in Sequence Name T51958Seg38 (SEQ ID NO: 1369) in Normal and Cancerous Colon Tissues

Expression of Homo sapiens PTK7 protein tyrosine kinase 7 (PTK7) transcripts detectable by or according to seg38, T51958seg38 amplicon (SEQ ID NO: 1369) and T51958seg38F (SEQ ID NO: 1367) and T51958seg38R (SEQ ID NO: 1368) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

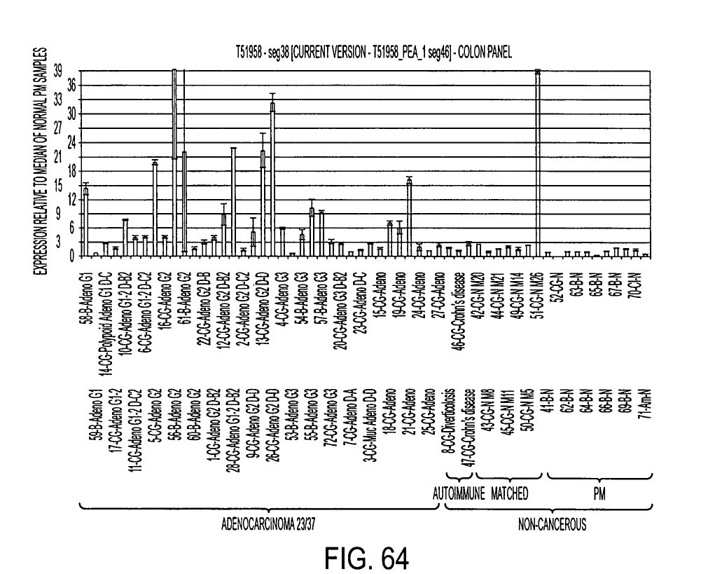

FIG. 64 is a histogram showing over expression of the above-indicated Homo sapiens PTK7 protein tyrosine kinase 7 (PTK7) transcripts in cancerous colon samples relative to the normal samples. (Values represent the average of duplicate experiments. Error bars indicate the minimal and maximal values obtained.) The number and percentage of samples that exhibit at least 3 fold over-expression, out of the total number of samples tested is indicated in the bottom.

As is evident from FIG. 64, the expression of Homo sapiens PTK7 protein tyrosine kinase 7 (PTK7) transcripts detectable by the above amplicon in cancer samples was significantly higher than in the non-cancerous samples (Sample Nos 41, 52, 62-67, 69-71, Table 1, “Tissue samples in testing panel”). Notably an over-expression of at least 3 fold was found in 23 out of 37 adenocarcinoma samples,

Statistical analysis was applied to verify the significance of these results, as described below.

The P value for the difference in the expression levels of Homo sapiens PTK7 protein tyrosine kinase 7 (PTK7) transcripts detectable by the above amplicon in colon cancer samples versus the normal tissue samples was determined by T test as 4.58E-04.

Threshold of 3 fold overexpression was found to differentiate between cancer and normal samples with P value of 1.97E-04 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: T51958seg38F forward primer (SEQ ID NO: 1367); and T51958seg38R reverse primer (SEQ ID NO: 1368).

The present invention also preferably encompasses any amplicon obtained through the use of any suitable primer pair; for example, for the above experiment, the following amplicon was obtained as a non-limiting illustrative example only of a suitable amplicon: T51958seg38 (SEQ ID NO: 1369).

Forward primer (SEQ ID NO: 1367):
GCTTGCCCTTTCATGTGGA
Reverse primer (SEQ ID NO: 1368):
TCACGATGAGACCTGACACTCTG
Amplicon (SEQ ID NO: 1369):
GCTTGCCCTTTCATGTGGAGCACTGTGATTGGACCCAAGTTGGCAAGAGT
GGAAGACCAGGGGACAGAACAGAAATCCCCATGGTGGCCAGAGTGTCAGG
TCTCATCGTGA

### Expression of Homo sapiens PTK7 Protein Tyrosine Kinase 7 (PTK7) T51958 Transcripts which are Detectable by Amplicon as Depicted in Sequence Name T51958Seg7 (SEQ ID NO: 1372) in Normal and Cancerous Colon Tissues

Expression of Homo sapiens PTK7 protein tyrosine kinase 7 (PTK7) transcripts detectable by or according to seg7, T51958seg7 amplicon (SEQ ID NO: 1372) and T51958seg7F (SEQ ID NO: 1370) and T51958seg7R (SEQ ID NO: 1371) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

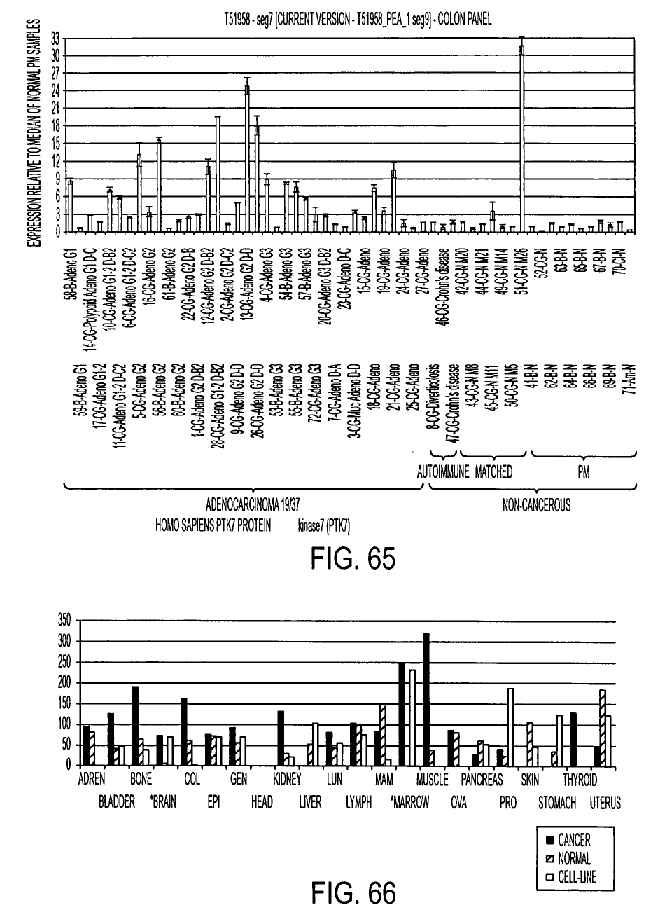

FIG. 65 is a histogram showing over expression of the above-indicated Homo sapiens PTK7 protein tyrosine kinase 7 (PTK7) transcripts in cancerous colon samples relative to the normal samples. (Values represent the average of duplicate experiments. Error bars indicate the minimal and maximal values obtained.) The number and percentage of samples that exhibit at least 3 fold over-expression, out of the total number of samples tested is indicated in the bottom.

As is evident from FIG. 65 the expression of Homo sapiens PTK7 protein tyrosine kinase 7 (PTK7) transcripts detectable by the above amplicon in cancer samples was significantly higher than in the non-cancerous samples (Sample Nos. 41, 52, 62-67, 69-71, Table 1, “Tissue samples in testing panel”). Notably an over-expression of at least 3 fold was found in 19 out of 37 adenocarcinoma samples,

Statistical analysis was applied to verify the significance of these results, as described below.

The P value for the difference in the expression levels of Homo sapiens PTK7 protein tyrosine kinase 7 (PTK7) transcripts detectable by the above amplicon in colon cancer samples versus the normal tissue samples was determined by T test as 1.74E-05.

Threshold of 3 fold overexpression was found to differentiate between cancer and normal samples with P value of 1.53E-03 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: T51958seg7F forward primer (SEQ ID NO: 1370); and T51958seg7R reverse primer (SEQ ID NO: 1371).

The present invention also preferably encompasses any amplicon obtained through the use of any suitable primer pair; for example, for the above experiment, the following amplicon was obtained as a non-limiting illustrative example only of a suitable amplicon: T51958seg7 (SEQ ID NO: 1372).

Forward primer (SEQ ID NO: 1370):
GTGCCCAGTCCCCCTGTC
Reverse primer (SEQ ID NO: 1371):
CCTGGCCCGTTTAACTGGA
Amplicon (SEQ ID NO: 1372):
GTGCCCAGTCCCCCTGTCAGACCCTCAATGACTGAGGCCTGGGGGATCCC
TCCCTTACCTCAGCTTCTCCCATTTCCAGTTAAACGGGCCAGG

### Description for Cluster Z17877

Cluster Z17877 features 9 transcript(s) and 17 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| Z17877_PEA_1_T0 | 1157 |
| Z17877_PEA_1_T2 | 1158 |
| Z17877_PEA_1_T3 | 1159 |
| Z17877_PEA_1_T4 | 1160 |
| Z17877_PEA_1_T6 | 1161 |
| Z17877_PEA_1_T7 | 1162 |
| Z17877_PEA_1_T8 | 1163 |
| Z17877_PEA_1_T11 | 1164 |
| Z17877_PEA_1_T12 | 1165 |

TABLE 2
Segments of interest

| Segment Name | SEQ ID NO: |
|---|---|
| Z17877_PEA_1_node_0 | 1166 |
| Z17877_PEA_1_node_3 | 1167 |
| Z17877_PEA_1_node_8 | 1168 |
| Z17877_PEA_1_node_9 | 1169 |
| Z17877_PEA_1_node_10 | 1170 |
| Z17877_PEA_1_node_11 | 1171 |
| Z17877_PEA_1_node_13 | 1172 |
| Z17877_PEA_1_node_15 | 1173 |
| Z17877_PEA_1_node_16 | 1174 |
| Z17877_PEA_1_node_18 | 1175 |
| Z17877_PEA_1_node_1 | 1176 |
| Z17877_PEA_1_node_2 | 1177 |
| Z17877_PEA_1_node_4 | 1178 |
| Z17877_PEA_1_node_5 | 1179 |
| Z17877_PEA_1_node_6 | 1180 |
| Z17877_PEA_1_node_14 | 1181 |
| Z17877_PEA_1_node_17 | 1182 |

TABLE 3
Proteins of interest

| | SEQ | |
|---|---|---|
| Protein Name | ID NO: | Corresponding Transcript(s) |
| Z17877_PEA_1_P1 | 1183 | Z17877_PEA_1_T0 (SEQ ID NO: |
| | | 1157); Z17877_PEA_1_T3 (SEQ ID |
| | | NO: 1159); Z17877_PEA_1_T7 |
| | | (SEQ ID NO: 1162) |
| Z17877_PEA_1_P2 | 1184 | Z17877_PEA_1_T6 (SEQ ID NO: |
| | | 1161); Z17877_PEA_1_T11 (SEQ ID |
| | | NO: 1164) |
| Z17877_PEA_1_P3 | 1185 | Z17877_PEA_1_T12 (SEQ ID NO: |
| | | 1165) |
| Z17877_PEA_1_P6 | 1186 | Z17877_PEA_1_T2 (SEQ ID NO: |
| | | 1158); Z17877_PEA_1_T4 (SEQ ID |
| | | NO: 1160); Z17877_PEA_1_T8 |
| | | (SEQ ID NO: 1163) |

Cluster Z17877 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 66 and Table 4. This cluster is overexpressed (at least at a minimum level) in the following pathological conditions: brain malignant tumors and malignant tumors involving the bone marrow.

TABLE 4
Normal tissue distribution

| Name of Tissue | Number |
|---|---|
| adrenal | 80 |
| bladder | 41 |
| bone | 64 |
| brain | 5 |
| colon | 63 |
| epithelial | 73 |
| general | 56 |
| head and neck | 0 |
| kidney | 31 |
| liver | 53 |
| lung | 44 |
| lymph nodes | 98 |
| breast | 149 |
| bone marrow | 0 |
| muscle | 40 |
| ovary | 80 |
| pancreas | 61 |
| prostate | 24 |
| skin | 107 |
| stomach | 36 |
| Thyroid | 0 |
| uterus | 186 |

TABLE 5
P values and ratios for expression in cancerous tissue

| Name of Tissue | P1 | P2 | SP1 | R3 | SP2 | R4 |
|---|---|---|---|---|---|---|
| adrenal | 5.2e−01 | 6.0e−01 | 6.2e−01 | 1.1 | 7.4e−01 | 0.9 |
| bladder | 5.4e−01 | 4.5e−01 | 2.8e−01 | 2.0 | 3.8e−01 | 1.7 |
| bone | 6.5e−02 | 3.1e−01 | 1.8e−01 | 2.3 | 5.6e−01 | 1.2 |
| brain | 2.5e−01 | 2.6e−01 | 1.5e−04 | 6.7 | 1.4e−05 | 6.9 |
| colon | 6.7e−02 | 1.0e−01 | 2.0e−01 | 1.9 | 4.0e−01 | 1.4 |
| epithelial | 3.4e−01 | 4.0e−01 | 4.1e−01 | 1.0 | 4.7e−01 | 0.9 |
| general | 3.2e−02 | 4.4e−02 | 2.8e−04 | 1.6 | 1.1e−03 | 1.4 |
| head and neck | 2.1e−01 | 3.3e−01 | 0.0e+00 | 0.0 | 0.0e+00 | 0.0 |
| kidney | 4.2e−01 | 5.2e−01 | 5.7e−02 | 2.6 | 1.5e−01 | 1.9 |
| liver | 8.2e−01 | 6.9e−01 | 1 | 0.5 | 5.1e−01 | 1.4 |
| lung | 3.4e−01 | 5.2e−01 | 2.9e−01 | 1.6 | 3.3e−01 | 1.2 |
| lymph nodes | 1.6e−01 | 2.9e−01 | 6.4e−01 | 1.1 | 7.0e−01 | 0.8 |
| breast | 7.2e−01 | 7.9e−01 | 8.6e−01 | 0.7 | 9.6e−01 | 0.5 |
| bone marrow | 4.3e−01 | 7.1e−02 | 1.5e−01 | 9.0 | 6.5e−03 | 7.5 |
| muscle | 6.0e−01 | 6.7e−01 | 2.6e−02 | 3.9 | 3.0e−01 | 1.3 |
| ovary | 5.8e−01 | 6.4e−01 | 6.1e−01 | 0.9 | 7.9e−01 | 0.7 |
| pancreas | 7.3e−01 | 7.3e−01 | 9.2e−01 | 0.5 | 8.7e−01 | 0.7 |
| prostate | 7.3e−01 | 7.6e−01 | 4.7e−01 | 1.4 | 1.0e−01 | 1.4 |
| skin | 5.7e−01 | 7.1e−01 | 1 | 0.1 | 9.7e−01 | 0.2 |
| stomach | 5.8e−01 | 5.9e−02 | 1 | 0.5 | 3.2e−01 | 1.8 |
| Thyroid | 2.9e−01 | 2.9e−01 | 4.4e−01 | 2.0 | 4.4e−01 | 2.0 |
| uterus | 6.9e−01 | 7.7e−01 | 1 | 0.3 | 9.7e−01 | 0.4 |

As noted above, cluster Z17877 features 9 transcript(s), which were listed in Table 1 above. A description of each variant protein according to the present invention is now provided.

Variant protein Z17877_PEA_{—}1_P1 (SEQ ID NO:1183) according to the present invention has an amino acid sequence as given at the end of the application; it is encoded by transcript(s) Z17877_PEA_{—}1_T0 (SEQ ID NO:1157). The location of the variant protein was determined according to results from a number of different software programs and analyses, including analyses from SignalP and other specialized programs. The variant protein is believed to be located as follows with regard to the cell: intracellularly. The protein localization is believed to be intracellularly because of manual inspection of known protein localization and/or gene structure.

Variant protein Z17877_PEA_{—}1_P1 (SEQ ID NO:1183) is encoded by the following transcript(s): Z17877_PEA_{—}1_T0 (SEQ ID NO:1157), for which the sequence(s) is/are given at the end of the application. The coding portion of transcript Z17877_PEA_{—}1_T0 (SEQ ID NO:1157) is shown in bold; this coding portion starts at position 1206 and ends at position 2522. The transcript also has the following SNPs as listed in Table 6 (given according to their position on the nucleotide sequence, with the alternative nucleic acid listed; the last column indicates whether the SNP is known or not; the presence of known SNPs in variant protein Z17877_PEA_{—}1_P1 (SEQ ID NO:1183) sequence provides support for the deduced sequence of this variant protein according to the present invention).

TABLE 6
Nucleic acid SNPs

| SNP position on | Alternative | Previously |
|---|---|---|
| nucleotide sequence | nucleic acid | known SNP? |
| 84 | C -> T | Yes |
| 209 | C -> G | Yes |
| 274 | G -> T | Yes |
| 457 | G -> C | Yes |
| 503 | A -> C | Yes |
| 628 | C -> T | Yes |
| 706 | G -> A | Yes |
| 771 | T -> C | Yes |
| 819 | C -> T | Yes |
| 930 | C -> | No |
| 1022 | T -> | No |
| 1065 | C -> T | Yes |
| 1237 | A -> G | Yes |
| 1376 | C -> G | No |
| 1379 | C -> G | No |
| 1440 | C -> G | No |
| 1653 | G -> A | No |
| 1683 | G -> T | Yes |
| 1713 | G -> A | Yes |
| 1898 | G -> A | Yes |
| 2123 | G -> A | No |
| 2170 | C -> T | Yes |
| 2297 | C -> | No |
| 2297 | C -> T | No |
| 2391 | -> T | No |
| 2431 | -> G | No |
| 2501 | A -> C | No |
| 2522 | G -> C | Yes |
| 2575 | A -> G | Yes |
| 2634 | T -> C | Yes |
| 2654 | A -> G | Yes |
| 2750 | T -> G | No |
| 2906 | T -> A | No |
| 2921 | T -> G | No |
| 2948 | T -> G | No |

Variant protein Z17877_PEA_{—}1_P2 (SEQ ID NO:1184) according to the present invention has an amino acid sequence as given at the end of the application; it is encoded by transcript(s) Z17877_PEA_{—}1_T6 (SEQ ID NO:1161) and Z17877_PEA_{—}1_T11 (SEQ ID NO:1164). The location of the variant protein was determined according to results from a number of different software programs and analyses, including analyses from SignalP and other specialized programs. The variant protein is believed to be located as follows with regard to the cell: intracellularly. The protein localization is believed to be intracellularly because of manual inspection of known protein localization and/or gene structure.

Variant protein Z17877_PEA_{—}1_P2 (SEQ ID NO:1184) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein Z17877_PEA_{—}1_P2 (SEQ ID NO:1184) sequence provides support for the deduced sequence of this variant protein according to the present invention).

TABLE 7
Amino acid mutations

| SNP position(s) on | Alternative | Previously |
|---|---|---|
| amino acid sequence | amino acid(s) | known SNP? |
| 11 | N -> S | Yes |
| 79 | P -> A | No |
| 150 | A -> T | No |
| 160 | G -> C | Yes |
| 170 | V -> I | Yes |
| 273 | I -> T | Yes |
| 352 | P -> L | Yes |

Variant protein Z17877_PEA_{—}1_P2 (SEQ ID NO:1184) is encoded by the following transcript(s): Z17877_PEA_{—}1_T6 (SEQ ID NO:1161) and Z17877_PEA_{—}1_T11 (SEQ ID NO:1164), for which the sequence(s) is/are given at the end of the application.

The coding portion of transcript Z17877_PEA_{—}1_T6 (SEQ ID NO:1161) is shown in bold; this coding portion starts at position 1206 and ends at position 2270. The transcript also has the following SNPs as listed in Table 8 (given according to their position on the nucleotide sequence, with the alternative nucleic acid listed; the last column indicates whether the SNP is known or not; the presence of known SNPs in variant protein Z17877_PEA_{—}1_P2 (SEQ ID NO:1184) sequence provides support for the deduced sequence of this variant protein according to the present invention).

TABLE 8
Nucleic acid SNPs

| SNP position on | Alternative | Previously |
|---|---|---|
| nucleotide sequence | nucleic acid | known SNP? |
| 84 | C -> T | Yes |
| 209 | C -> G | Yes |
| 274 | G -> T | Yes |
| 457 | G -> C | Yes |
| 503 | A -> C | Yes |
| 628 | C -> T | Yes |
| 706 | G -> A | Yes |
| 771 | T -> C | Yes |
| 819 | C -> T | Yes |
| 930 | C -> | No |
| 1022 | T -> | No |
| 1065 | C -> T | Yes |
| 1237 | A -> G | Yes |
| 1376 | C -> G | No |
| 1379 | C -> G | No |
| 1440 | C -> G | No |
| 1653 | G -> A | No |
| 1683 | G -> T | Yes |
| 1713 | G -> A | Yes |
| 1898 | G -> A | Yes |
| 2023 | T -> C | Yes |
| 2260 | C -> T | Yes |
| 2453 | G -> C | Yes |
| 2482 | G -> A | Yes |
| 2553 | T -> | Yes |
| 2603 | T -> | Yes |
| 2612 | T -> | Yes |
| 2875 | G -> A | Yes |
| 2938 | G -> A | Yes |
| 3295 | A -> G | Yes |
| 3499 | G -> A | No |
| 3546 | C -> T | Yes |
| 3673 | C -> | No |
| 3673 | C -> T | No |
| 3767 | -> T | No |
| 3807 | -> G | No |
| 3877 | A -> C | No |
| 3898 | G -> C | Yes |
| 3951 | A -> G | Yes |
| 4010 | T -> C | Yes |
| 4030 | A -> G | Yes |
| 4126 | T -> G | No |
| 4282 | T -> A | No |
| 4297 | T -> G | No |
| 4324 | T -> G | No |

The coding portion of transcript Z17877_PEA_{—}1_T11 (SEQ ID NO:1164) is shown in bold; this coding portion starts at position 602 and ends at position 1666. The transcript also has the following SNPs as listed in Table 9 (given according to their position on the nucleotide sequence, with the alternative nucleic acid listed; the last column indicates whether the SNP is known or not; the presence of known SNPs in variant protein Z17877_PEA_{—}1_P2 (SEQ ID NO:1184) sequence provides support for the deduced sequence of this variant protein according to the present invention).

TABLE 9
Nucleic acid SNPs

| SNP position on | Alternative | Previously |
|---|---|---|
| nucleotide sequence | nucleic acid | known SNP? |
| 84 | C -> T | Yes |
| 209 | C -> G | Yes |
| 274 | G -> T | Yes |
| 418 | T -> | No |
| 461 | C -> T | Yes |
| 633 | A -> G | Yes |
| 772 | C -> G | No |
| 775 | C -> G | No |
| 836 | C -> G | No |
| 1049 | G -> A | No |
| 1079 | G -> T | Yes |
| 1109 | G -> A | Yes |
| 1294 | G -> A | Yes |
| 1419 | T -> C | Yes |
| 1656 | C -> T | Yes |
| 1849 | G -> C | Yes |
| 1878 | G -> A | Yes |
| 1949 | T -> | Yes |
| 1999 | T -> | Yes |
| 2008 | T -> | Yes |
| 2271 | G -> A | Yes |
| 2334 | G -> A | Yes |
| 2691 | A -> G | Yes |
| 2868 | G -> A | No |
| 2915 | C -> T | Yes |
| 3042 | C -> | No |
| 3042 | C -> T | No |
| 3136 | -> T | No |
| 3176 | -> G | No |
| 3246 | A -> C | No |
| 3267 | G -> C | Yes |
| 3320 | A -> G | Yes |
| 3379 | T -> C | Yes |
| 3399 | A -> G | Yes |
| 3495 | T -> G | No |
| 3651 | T -> A | No |
| 3666 | T -> G | No |
| 3693 | T -> G | No |

Variant protein Z17877_PEA_{—}1_P3 (SEQ ID NO:1185) according to the present invention has an amino acid sequence as given at the end of the application; it is encoded by transcript(s) Z17877_PEA_{—}1_T12 (SEQ ID NO:1165). The location of the variant protein was determined according to results from a number of different software programs and analyses, including analyses from SignalP and other specialized programs. The variant protein is believed to be located as follows with regard to the cell: intracellularly. The protein localization is believed to be intracellularly because of manual inspection of known protein localization and/or gene structure.

Variant protein Z17877_PEA_{—}1_P3 (SEQ ID NO:1185) also has the following non-silent SNPs (Single Nucleotide Polymorphisms) as listed in Table 10, (given according to their position(s) on the amino acid sequence, with the alternative amino acid(s) listed; the last column indicates whether the SNP is known or not; the presence of known SNPs in variant protein Z17877_PEA_{—}1_P3 (SEQ ID NO:1185) sequence provides support for the deduced sequence of this variant protein according to the present invention).

TABLE 10
Amino acid mutations

| SNP position(s) on | Alternative | Previously |
|---|---|---|
| amino acid sequence | amino acid(s) | known SNP? |
| 11 | N -> S | Yes |
| 79 | P -> A | No |
| 150 | A -> T | No |
| 160 | G -> C | Yes |
| 170 | V -> I | Yes |
| 331 | A -> V | Yes |
| 373 | R -> | No |
| 441 | E -> D | No |

Variant protein Z17877_PEA_{—}1_P3 (SEQ ID NO:1185) is encoded by the following transcript(s): Z17877_PEA_{—}1_T12 (SEQ ID NO:1165), for which the sequence(s) is/are given at the end of the application. The coding portion of transcript Z17877_PEA_{—}1_T12 (SEQ ID NO:1165) is shown in bold; this coding portion starts at position 602 and ends at position 1945. The transcript also has the following SNPs as listed in Table 11 (given according to their position on the nucleotide sequence, with the alternative nucleic acid listed; the last column indicates whether the SNP is known or not; the presence of known SNPs in variant protein Z17877_PEA_{—}1_P3 (SEQ ID NO:1185) sequence provides support for the deduced sequence of this variant protein according to the present invention).

TABLE 11
Nucleic acid SNPs

| SNP position on | Alternative | Previously |
|---|---|---|
| nucleotide sequence | nucleic acid | known SNP? |
| 84 | C -> T | Yes |
| 209 | C -> G | Yes |
| 274 | G -> T | Yes |
| 418 | T -> | No |
| 461 | C -> T | Yes |
| 633 | A -> G | Yes |
| 772 | C -> G | No |
| 775 | C -> G | No |
| 836 | C -> G | No |
| 1049 | G -> A | No |
| 1079 | G -> T | Yes |
| 1109 | G -> A | Yes |
| 1294 | G -> A | Yes |
| 1546 | G -> A | No |
| 1593 | C -> T | Yes |
| 1720 | C -> | No |
| 1720 | C -> T | No |
| 1814 | -> T | No |
| 1854 | -> G | No |
| 1924 | A -> C | No |
| 1945 | G -> C | Yes |
| 1998 | A -> G | Yes |
| 2057 | T -> C | Yes |
| 2077 | A -> G | Yes |
| 2173 | T -> G | No |
| 2329 | T -> A | No |
| 2344 | T -> G | No |
| 2371 | T -> G | No |

Variant protein Z17877_PEA_{—}1_P6 (SEQ ID NO:1186) according to the present invention has an amino acid sequence as given at the end of the application; it is encoded by transcript(s) Z17877_PEA_{—}1_T2 (SEQ ID NO:1158), Z17877_PEA_{—}1_T4 (SEQ ID NO:1160) and Z17877_PEA_{—}1_T8 (SEQ ID NO:1163). The location of the variant protein was determined according to results from a number of different software programs and analyses, including analyses from SignalP and other specialized programs. The variant protein is believed to be located as follows with regard to the cell: unknown.

Variant protein Z17877_PEA_{—}1_P6 (SEQ ID NO:1186) also has the following non-silent SNPs (Single Nucleotide Polymorphisms) as listed in Table 12, (given according to their position(s) on the amino acid sequence, with the alternative amino acid(s) listed; the last column indicates whether the SNP is known or not; the presence of known SNPs in variant protein Z17877_PEA_{—}1_P6 (SEQ ID NO:1186) sequence provides support for the deduced sequence of this variant protein according to the present invention).

TABLE 12
Amino acid mutations

| SNP position(s) on | Alternative | Previously |
|---|---|---|
| amino acid sequence | amino acid(s) | known SNP? |
| 57 | A -> G | Yes |
| 79 | G -> C | Yes |

Variant protein Z17877_PEA_{—}1_P6 (SEQ ID NO:1186) is encoded by the following transcript(s): Z17877_PEA_{—}1_T2 (SEQ ID NO:1158), Z17877_PEA_{—}1_T4 (SEQ ID NO:1160) and Z17877_PEA_{—}1_T8 (SEQ ID NO:1163), for which the sequence(s) is/are given at the end of the application.

The coding portion of transcript Z17877_PEA_{—}1_T2 (SEQ ID NO:1158) is shown in bold; this coding portion starts at position 40 and ends at position 381. The transcript also has the following SNPs as listed in Table 13 (given according to their position on the nucleotide sequence, with the alternative nucleic acid listed; the last column indicates whether the SNP is known or not; the presence of known SNPs in variant protein Z17877_PEA_{—}1_P6 (SEQ ID NO:1186) sequence provides support for the deduced sequence of this variant protein according to the present invention).

TABLE 13
Nucleic acid SNPs

| SNP position on | Alternative | Previously |
|---|---|---|
| nucleotide sequence | nucleic acid | known SNP? |
| 84 | C -> T | Yes |
| 209 | C -> G | Yes |
| 274 | G -> T | Yes |
| 457 | G -> C | Yes |
| 503 | A -> C | Yes |
| 628 | C -> T | Yes |
| 706 | G -> A | Yes |
| 771 | T -> C | Yes |
| 819 | C -> T | Yes |
| 930 | C -> | No |
| 1022 | T -> | No |
| 1065 | C -> T | Yes |
| 1344 | G -> T | Yes |
| 1361 | C -> G | Yes |
| 1413 | G -> A | Yes |
| 1418 | G -> A | Yes |
| 1790 | C -> T | Yes |
| 2059 | G -> A | Yes |
| 2244 | G -> A | Yes |
| 2451 | T -> G | No |
| 2460 | T -> A | Yes |
| 2500 | G -> | No |
| 2533 | C -> T | Yes |
| 2581 | C -> T | Yes |
| 2734 | C -> G | Yes |
| 2861 | A -> G | Yes |
| 3000 | C -> G | No |
| 3003 | C -> G | No |
| 3064 | C -> G | No |
| 3277 | G -> A | No |
| 3307 | G -> T | Yes |
| 3337 | G -> A | Yes |
| 3522 | G -> A | Yes |
| 3747 | G -> A | No |
| 3794 | C -> T | Yes |
| 3921 | C -> | No |
| 3921 | C -> T | No |
| 4015 | -> T | No |
| 4055 | -> G | No |
| 4125 | A -> C | No |
| 4146 | G -> C | Yes |
| 4199 | A -> G | Yes |
| 4258 | T -> C | Yes |
| 4278 | A -> G | Yes |
| 4374 | T -> G | No |
| 4530 | T -> A | No |
| 4545 | T -> G | No |
| 4572 | T -> G | No |

The coding portion of transcript Z17877_PEA_{—}1_T4 (SEQ ID NO:1160) is shown in bold; this coding portion starts at position 40 and ends at position 381. The transcript also has the following SNPs as listed in Table 14 (given according to their position on the nucleotide sequence, with the alternative nucleic acid listed; the last column indicates whether the SNP is known or not; the presence of known SNPs in variant protein Z17877_PEA_{—}1_P6 (SEQ ID NO:1186) sequence provides support for the deduced sequence of this variant protein according to the present invention).

TABLE 14
Nucleic acid SNPs

| SNP position on | Alternative | Previously |
|---|---|---|
| nucleotide sequence | nucleic acid | known SNP? |
| 84 | C -> T | Yes |
| 209 | C -> G | Yes |
| 274 | G -> T | Yes |
| 457 | G -> C | Yes |
| 503 | A -> C | Yes |
| 628 | C -> T | Yes |
| 706 | G -> A | Yes |
| 844 | C -> | No |
| 936 | T -> | No |
| 979 | C -> T | Yes |
| 1258 | G -> T | Yes |
| 1275 | C -> G | Yes |
| 1327 | G -> A | Yes |
| 1332 | G -> A | Yes |
| 1704 | C -> T | Yes |
| 1973 | G -> A | Yes |
| 2158 | G -> A | Yes |
| 2365 | T -> G | No |
| 2374 | T -> A | Yes |
| 2414 | G -> | No |
| 2447 | C -> T | Yes |
| 2495 | C -> T | Yes |
| 2648 | C -> G | Yes |
| 2775 | A -> G | Yes |
| 2914 | C -> G | No |
| 2917 | C -> G | No |
| 2978 | C -> G | No |
| 3191 | G -> A | No |
| 3221 | G -> T | Yes |
| 3251 | G -> A | Yes |
| 3436 | G -> A | Yes |
| 3661 | G -> A | No |
| 3708 | C -> T | Yes |
| 3835 | C -> | No |
| 3835 | C -> T | No |
| 3929 | -> T | No |
| 3969 | -> G | No |
| 4039 | A -> C | No |
| 4060 | G -> C | Yes |
| 4113 | A -> G | Yes |
| 4172 | T -> C | Yes |
| 4192 | A -> G | Yes |
| 4288 | T -> G | No |
| 4444 | T -> A | No |
| 4459 | T -> G | No |
| 4486 | T -> G | No |

The coding portion of transcript Z17877_PEA_{—}1_T8 (SEQ ID NO:1163) is shown in bold; this coding portion starts at position 40 and ends at position 381. The transcript also has the following SNPs as listed in Table 15 (given according to their position on the nucleotide sequence, with the alternative nucleic acid listed; the last column indicates whether the SNP is known or not; the presence of known SNPs in variant protein Z17877_PEA_{—}1_P6 (SEQ ID NO:1186) sequence provides support for the deduced sequence of this variant protein according to the present invention).

TABLE 15
Nucleic acid SNPs

| SNP position on | Alternative | Previously |
|---|---|---|
| nucleotide sequence | nucleic acid | known SNP? |
| 84 | C -> T | Yes |
| 209 | C -> G | Yes |
| 274 | G -> T | Yes |
| 457 | G -> C | Yes |
| 503 | A -> C | Yes |
| 628 | C -> T | Yes |
| 706 | G -> A | Yes |
| 771 | T -> C | Yes |
| 819 | C -> T | Yes |
| 930 | C -> | No |
| 1022 | T -> | No |
| 1065 | C -> T | Yes |
| 1237 | A -> G | Yes |
| 1376 | C -> G | No |
| 1379 | C -> G | No |
| 1440 | C -> G | No |
| 1653 | G -> A | No |
| 1683 | G -> T | Yes |
| 1713 | G -> A | Yes |
| 1898 | G -> A | Yes |
| 2150 | G -> A | No |
| 2197 | C -> T | Yes |
| 2324 | C -> | No |
| 2324 | C -> T | No |
| 2418 | -> T | No |
| 2458 | -> G | No |
| 2528 | A -> C | No |
| 2549 | G -> C | Yes |
| 2602 | A -> G | Yes |
| 2661 | T -> C | Yes |
| 2681 | A -> G | Yes |
| 2777 | T -> G | No |
| 2933 | T -> A | No |
| 2948 | T -> G | No |
| 2975 | T -> G | No |

As noted above, cluster Z17877 features 17 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster Z17877_PEA_{—}1_node_{—}0 (SEQ ID NO:1166) according to the present invention is supported by 4 libraries. The number of libraries was determined as previously described. This segment can be found in the following transcript(s): Z17877_PEA_{—}1_T0 (SEQ ID NO:1157), Z17877_PEA_{—}1_T2 (SEQ ID NO:1158), Z17877_PEA_{—}1_T3 (SEQ ID NO:1159), Z17877_PEA_{—}1_T4 (SEQ ID NO:1160), Z17877_PEA_{—}1_T6 (SEQ ID NO:1161), Z17877_PEA_{—}1_T7 (SEQ ID NO:1162), Z17877_PEA_{—}1_T8 (SEQ ID NO:1163), Z17877_PEA_{—}1_T11 (SEQ ID NO:1164) and Z17877_PEA_{—}1_T12 (SEQ ID NO:1165). Table 16 below describes the starting and ending position of this segment on each transcript.

TABLE 16
Segment location on transcripts

| | Segment | Segment |
|---|---|---|
| | starting | ending |
| Transcript name | position | position |
| Z17877_PEA_1_T0 (SEQ ID NO: 1157) | 1 | 330 |
| Z17877_PEA_1_T2 (SEQ ID NO: 1158) | 1 | 330 |
| Z17877_PEA_1_T3 (SEQ ID NO: 1159) | 1 | 330 |
| Z17877_PEA_1_T4 (SEQ ID NO: 1160) | 1 | 330 |
| Z17877_PEA_1_T6 (SEQ ID NO: 1161) | 1 | 330 |
| Z17877_PEA_1_T7 (SEQ ID NO: 1162) | 1 | 330 |
| Z17877_PEA_1_T8 (SEQ ID NO: 1163) | 1 | 330 |
| Z17877_PEA_1_T11 (SEQ ID NO: 1164) | 1 | 330 |
| Z17877_PEA_1_T12 (SEQ ID NO: 1165) | 1 | 330 |

Segment cluster Z17877_PEA_{—}1_node_{—}3 (SEQ ID NO:1167) according to the present invention is supported by 23 libraries. The number of libraries was determined as previously described. This segment can be found in the following transcript(s): Z17877_PEA_{—}1_T0 (SEQ ID NO:1157), Z17877_PEA_{—}1_T2 (SEQ ID NO:1158), Z17877_PEA_{—}1_T3 (SEQ ID NO:1159), Z17877_PEA_{—}1_T4 (SEQ ID NO:1160), Z17877_PEA_{—}1_T6 (SEQ ID NO:1161) and Z17877_PEA_{—}1_T8 (SEQ ID NO:1163). Table 17 below describes the starting and ending position of this segment on each transcript.

TABLE 17
Segment location on transcripts

| | Segment | Segment |
|---|---|---|
| | starting | ending |
| Transcript name | position | position |
| Z17877_PEA_1_T0 (SEQ ID NO: 1157) | 404 | 755 |
| Z17877_PEA_1_T2 (SEQ ID NO: 1158) | 404 | 755 |
| Z17877_PEA_1_T3 (SEQ ID NO: 1159) | 404 | 755 |
| Z17877_PEA_1_T4 (SEQ ID NO: 1160) | 404 | 755 |
| Z17877_PEA_1_T6 (SEQ ID NO: 1161) | 404 | 755 |
| Z17877_PEA_1_T8 (SEQ ID NO: 1163) | 404 | 755 |

Segment cluster Z17877_PEA_{—}1_node_{—}8 (SEQ ID NO:1168) according to the present invention is supported by 100 libraries. The number of libraries was determined as previously described. This segment can be found in the following transcript(s): Z17877_PEA_{—}1_T0 (SEQ ID NO:1157), Z17877_PEA_{—}1_T2 (SEQ ID NO:1158), Z17877_PEA_{—}1_T3 (SEQ ID NO:1159), Z17877_PEA_{—}1_T4 (SEQ ID NO:1160), Z17877_PEA_{—}1_T6 (SEQ ID NO:1161) and Z17877_PEA_{—}1_T8 (SEQ ID NO:1163). Table 18 below describes the starting and ending position of this segment on each transcript.

TABLE 18
Segment location on transcripts

| | Segment | Segment |
|---|---|---|
| | starting | ending |
| Transcript name | position | position |
| Z17877_PEA_1_T0 (SEQ ID NO: 1157) | 862 | 1007 |
| Z17877_PEA_1_T2 (SEQ ID NO: 1158) | 862 | 1007 |
| Z17877_PEA_1_T3 (SEQ ID NO: 1159) | 862 | 1007 |
| Z17877_PEA_1_T4 (SEQ ID NO: 1160) | 776 | 921 |
| Z17877_PEA_1_T6 (SEQ ID NO: 1161) | 862 | 1007 |
| Z17877_PEA_1_T8 (SEQ ID NO: 1163) | 862 | 1007 |

Segment cluster Z17877_PEA_{—}1_node_{—}9 (SEQ ID NO:1169) according to the present invention is supported by 110 libraries. The number of libraries was determined as previously described. This segment can be found in the following transcript(s): Z17877_PEA_{—}1_T0 (SEQ ID NO:1157), Z17877_PEA_{—}1_T2 (SEQ ID NO:1158), Z17877_PEA_{—}1_T3 (SEQ ID NO:1159), Z17877_PEA_{—}1_T4 (SEQ ID NO:1160), Z17877_PEA_{—}1_T6 (SEQ ID NO:1161), Z17877_PEA_{—}1_T7 (SEQ ID NO:1162), Z17877_PEA_{—}1_T8 (SEQ ID NO:1163), Z17877_PEA_{—}1_T11 (SEQ ID NO:1164) and Z17877_PEA_{—}1_T12 (SEQ ID NO:1165). Table 19 below describes the starting and ending position of this segment on each transcript.

TABLE 19
Segment location on transcripts

| | Segment | Segment |
|---|---|---|
| | starting | ending |
| Transcript name | position | position |
| Z17877_PEA_1_T0 (SEQ ID NO: 1157) | 1008 | 1190 |
| Z17877_PEA_1_T2 (SEQ ID NO: 1158) | 1008 | 1190 |
| Z17877_PEA_1_T3 (SEQ ID NO: 1159) | 1008 | 1190 |
| Z17877_PEA_1_T4 (SEQ ID NO: 1160) | 922 | 1104 |
| Z17877_PEA_1_T6 (SEQ ID NO: 1161) | 1008 | 1190 |
| Z17877_PEA_1_T7 (SEQ ID NO: 1162) | 404 | 586 |
| Z17877_PEA_1_T8 (SEQ ID NO: 1163) | 1008 | 1190 |
| Z17877_PEA_1_T11 (SEQ ID NO: 1164) | 404 | 586 |
| Z17877_PEA_1_T12 (SEQ ID NO: 1165) | 404 | 586 |

Segment cluster Z17877_PEA_{—}1_node_{—}10 (SEQ ID NO:1170) according to the present invention is supported by 8 libraries. The number of libraries was determined as previously described. This segment can be found in the following transcript(s): Z17877_PEA_{—}1_T2 (SEQ ID NO:1158) and Z17877_PEA_{—}1_T4 (SEQ ID NO:1160). Table 20 below describes the starting and ending position of this segment on each transcript.

TABLE 20
Segment location on transcripts

| | Segment | Segment |
|---|---|---|
| | starting | ending |
| Transcript name | position | position |
| Z17877_PEA_1_T2 (SEQ ID NO: 1158) | 1191 | 1520 |
| Z17877_PEA_1_T4 (SEQ ID NO: 1160) | 1105 | 1434 |

Segment cluster Z17877_PEA_{—}1_node_{—}11 (SEQ ID NO:1171) according to the present invention is supported by 23 libraries. The number of libraries was determined as previously described. This segment can be found in the following transcript(s): Z17877_PEA_{—}1_T2 (SEQ ID NO:1158) and Z17877_PEA_{—}1_T4 (SEQ ID NO:1160). Table 21 below describes the starting and ending position of this segment on each transcript.

TABLE 21
Segment location on transcripts

| | Segment | Segment |
|---|---|---|
| | starting | ending |
| Transcript name | position | position |
| Z17877_PEA_1_T2 (SEQ ID NO: 1158) | 1521 | 2814 |
| Z17877_PEA_1_T4 (SEQ ID NO: 1160) | 1435 | 2728 |

Segment cluster Z17877_PEA_{—}1_node_{—}13 (SEQ ID NO:1172) according to the present invention is supported by 108 libraries. The number of libraries was determined as previously described. This segment can be found in the following transcript(s): Z17877_PEA_{—}1_T0 (SEQ ID NO:1157), Z17877_PEA_{—}1_T2 (SEQ ID NO:1158), Z17877_PEA_{—}1_T3 (SEQ ID NO:1159), Z17877_PEA_{—}1_T4 (SEQ ID NO:1160), Z17877_PEA_{—}1_T6 (SEQ ID NO:1161), Z17877_PEA_{—}1_T7 (SEQ ID NO:1162), Z17877_PEA_{—}1_T8 (SEQ ID NO:1163), Z17877_PEA_{—}1_T11 (SEQ ID NO:1164) and Z17877_PEA_{—}1_T12 (SEQ ID NO:1165). Table 22 below describes the starting and ending position of this segment on each transcript.

TABLE 22
Segment location on transcripts

| | Segment | Segment |
|---|---|---|
| | starting | ending |
| Transcript name | position | position |
| Z17877_PEA_1_T0 (SEQ ID NO: 1157) | 1191 | 1402 |
| Z17877_PEA_1_T2 (SEQ ID NO: 1158) | 2815 | 3026 |
| Z17877_PEA_1_T3 (SEQ ID NO: 1159) | 1191 | 1402 |
| Z17877_PEA_1_T4 (SEQ ID NO: 1160) | 2729 | 2940 |
| Z17877_PEA_1_T6 (SEQ ID NO: 1161) | 1191 | 1402 |
| Z17877_PEA_1_T7 (SEQ ID NO: 1162) | 587 | 798 |
| Z17877_PEA_1_T8 (SEQ ID NO: 1163) | 1191 | 1402 |
| Z17877_PEA_1_T11 (SEQ ID NO: 1164) | 587 | 798 |
| Z17877_PEA_1_T12 (SEQ ID NO: 1165) | 587 | 798 |

Segment cluster Z17877_PEA_{—}1_node_{—}15 (SEQ ID NO:1173) according to the present invention is supported by 139 libraries. The number of libraries was determined as previously described. This segment can be found in the following transcript(s): Z17877_PEA_{—}1_T0 (SEQ ID NO:1157), Z17877_PEA_{—}1_T2 (SEQ ID NO:1158), Z17877_PEA_{—}1_T3 (SEQ ID NO:1159), Z17877_PEA_{—}1_T4 (SEQ ID NO:1160), Z17877_PEA_{—}1_T6 (SEQ ID NO:1161), Z17877_PEA_{—}1_T7 (SEQ ID NO:1162), Z17877_PEA_{—}1_T8 (SEQ ID NO:1163), Z17877_PEA_{—}1_T11 (SEQ ID NO:1164) and Z17877_PEA_{—}1_T12 (SEQ ID NO:1165). Table 23 below describes the starting and ending position of this segment on each transcript.

TABLE 23
Segment location on transcripts

| | Segment | Segment |
|---|---|---|
| | starting | ending |
| Transcript name | position | position |
| Z17877_PEA_1_T0 (SEQ ID NO: 1157) | 1481 | 1962 |
| Z17877_PEA_1_T2 (SEQ ID NO: 1158) | 3105 | 3586 |
| Z17877_PEA_1_T3 (SEQ ID NO: 1159) | 1481 | 1962 |
| Z17877_PEA_1_T4 (SEQ ID NO: 1160) | 3019 | 3500 |
| Z17877_PEA_1_T6 (SEQ ID NO: 1161) | 1481 | 1962 |
| Z17877_PEA_1_T7 (SEQ ID NO: 1162) | 877 | 1358 |
| Z17877_PEA_1_T8 (SEQ ID NO: 1163) | 1481 | 1962 |
| Z17877_PEA_1_T11 (SEQ ID NO: 1164) | 877 | 1358 |
| Z17877_PEA_1_T12 (SEQ ID NO: 1165) | 877 | 1358 |

Segment cluster Z17877_PEA_{—}1_node_{—}16 (SEQ ID NO:1174) according to the present invention is supported by 21 libraries. The number of libraries was determined as previously described. This segment can be found in the following transcript(s): Z17877_PEA_{—}1_T6 (SEQ ID NO:1161) and Z17877_PEA_{—}1_T11 (SEQ ID NO:1164). Table 24 below describes the starting and ending position of this segment on each transcript.

TABLE 24
Segment location on transcripts

| | Segment | Segment |
|---|---|---|
| | starting | ending |
| Transcript name | position | position |
| Z17877_PEA_1_T6 (SEQ ID NO: 1161) | 1963 | 3311 |
| Z17877_PEA_1_T11 (SEQ ID NO: 1164) | 1359 | 2707 |

Segment cluster Z17877_PEA_{—}1_node_{—}18 (SEQ ID NO:1175) according to the present invention is supported by 263 libraries. The number of libraries was determined as previously described. This segment can be found in the following transcript(s): Z17877_PEA_{—}1_T0 (SEQ ID NO:1157), Z17877_PEA_{—}1_T2 (SEQ ID NO:1158), Z17877_PEA_{—}1_T3 (SEQ ID NO:1159), Z17877_PEA_{—}1_T4 (SEQ ID NO:1160), Z17877_PEA_{—}1_T6 (SEQ ID NO:1161), Z17877_PEA_{—}1_T7 (SEQ ID NO:1162), Z17877_PEA_{—}1_T8 (SEQ ID NO:1163), Z17877_PEA_{—}1_T11 (SEQ ID NO:1164) and Z17877_PEA_{—}1_T12 (SEQ ID NO:1165). Table 25 below describes the starting and ending position of this segment on each transcript.

TABLE 25
Segment location on transcripts

| | Segment | Segment |
|---|---|---|
| | starting | ending |
| Transcript name | position | position |
| Z17877_PEA_1_T0 (SEQ ID NO: 1157) | 1963 | 3001 |
| Z17877_PEA_1_T2 (SEQ ID NO: 1158) | 3587 | 4625 |
| Z17877_PEA_1_T3 (SEQ ID NO: 1159) | 1963 | 2841 |
| Z17877_PEA_1_T4 (SEQ ID NO: 1160) | 3501 | 4539 |
| Z17877_PEA_1_T6 (SEQ ID NO: 1161) | 3339 | 4377 |
| Z17877_PEA_1_T7 (SEQ ID NO: 1162) | 1359 | 2397 |
| Z17877_PEA_1_T8 (SEQ ID NO: 1163) | 1990 | 3028 |
| Z17877_PEA_1_T11 (SEQ ID NO: 1164) | 2708 | 3746 |
| Z17877_PEA_1_T12 (SEQ ID NO: 1165) | 1386 | 2424 |

According to an optional embodiment of the present invention, short segments related to the above cluster are also provided. These segments are up to about 120 bp in length, and so are included in a separate description.

Segment cluster Z17877_PEA_{—}1_node_{—}1 (SEQ ID NO:1176) according to the present invention is supported by 3 libraries. The number of libraries was determined as previously described. This segment can be found in the following transcript(s): Z17877_PEA_{—}1_T0 (SEQ ID NO:1157), Z17877_PEA_{—}1_T2 (SEQ ID NO:1158), Z17877_PEA_{—}1_T3 (SEQ ID NO:1159), Z17877_PEA_{—}1_T4 (SEQ ID NO:1160), Z17877_PEA_{—}1_T6 (SEQ ID NO:1161), Z17877_PEA_{—}1_T7 (SEQ ID NO:1162), Z17877_PEA_{—}1_T8 (SEQ ID NO:1163), Z17877_PEA_{—}1_T11 (SEQ ID NO:1164) and Z17877_PEA_{—}1_T12 (SEQ ID NO:1165). Table 26 below describes the starting and ending position of this segment on each transcript.

TABLE 26
Segment location on transcripts

| | Segment | Segment |
|---|---|---|
| | starting | ending |
| Transcript name | position | position |
| Z17877_PEA_1_T0 (SEQ ID NO: 1157) | 331 | 378 |
| Z17877_PEA_1_T2 (SEQ ID NO: 1158) | 331 | 378 |
| Z17877_PEA_1_T3 (SEQ ID NO: 1159) | 331 | 378 |
| Z17877_PEA_1_T4 (SEQ ID NO: 1160) | 331 | 378 |
| Z17877_PEA_1_T6 (SEQ ID NO: 1161) | 331 | 378 |
| Z17877_PEA_1_T7 (SEQ ID NO: 1162) | 331 | 378 |
| Z17877_PEA_1_T8 (SEQ ID NO: 1163) | 331 | 378 |
| Z17877_PEA_1_T11 (SEQ ID NO: 1164) | 331 | 378 |
| Z17877_PEA_1_T12 (SEQ ID NO: 1165) | 331 | 378 |

Segment cluster Z17877_PEA_{—}1_node_{—}2 (SEQ ID NO:1177) according to the present invention can be found in the following transcript(s): Z17877_PEA_{—}1_T0 (SEQ ID NO:1157), Z17877_PEA_{—}1_T2 (SEQ ID NO:1158), Z17877_PEA_{—}1_T3 (SEQ ID NO:1159), Z17877_PEA_{—}1_T4 (SEQ ID NO:1160), Z17877_PEA_{—}1_T6 (SEQ ID NO:1161), Z17877_PEA_{—}1_T7 (SEQ ID NO:1162), Z17877_PEA_{—}1_T8 (SEQ ID NO:1163), Z17877_PEA_{—}1_T11 (SEQ ID NO:1164) and Z17877_PEA_{—}1_T12 (SEQ ID NO:1165). Table 27 below describes the starting and ending position of this segment on each transcript.

TABLE 27
Segment location on transcripts

| | Segment | Segment |
|---|---|---|
| | starting | ending |
| Transcript name | position | position |
| Z17877_PEA_1_T0 (SEQ ID NO: 1157) | 379 | 403 |
| Z17877_PEA_1_T2 (SEQ ID NO: 1158) | 379 | 403 |
| Z17877_PEA_1_T3 (SEQ ID NO: 1159) | 379 | 403 |
| Z17877_PEA_1_T4 (SEQ ID NO: 1160) | 379 | 403 |
| Z17877_PEA_1_T6 (SEQ ID NO: 1161) | 379 | 403 |
| Z17877_PEA_1_T7 (SEQ ID NO: 1162) | 379 | 403 |
| Z17877_PEA_1_T8 (SEQ ID NO: 1163) | 379 | 403 |
| Z17877_PEA_1_T11 (SEQ ID NO: 1164) | 379 | 403 |
| Z17877_PEA_1_T12 (SEQ ID NO: 1165) | 379 | 403 |

Segment cluster Z17877_PEA_{—}1_node_{—}4 (SEQ ID NO:1178) according to the present invention can be found in the following transcript(s): Z17877_PEA_{—}1_T0 (SEQ ID NO:1157), Z17877_PEA_{—}1_T2 (SEQ ID NO:1158), Z17877_PEA_{—}1_T3 (SEQ ID NO:1159), Z17877_PEA_{—}1_T4 (SEQ ID NO:1160), Z17877_PEA_{—}1_T6 (SEQ ID NO:1161) and Z17877_PEA_{—}1_T8 (SEQ ID NO:1163). Table 28 below describes the starting and ending position of this segment on each transcript.

TABLE 28
Segment location on transcripts

| | Segment | Segment |
|---|---|---|
| | starting | ending |
| Transcript name | position | position |
| Z17877_PEA_1_T0 (SEQ ID NO: 1157) | 756 | 763 |
| Z17877_PEA_1_T2 (SEQ ID NO: 1158) | 756 | 763 |
| Z17877_PEA_1_T3 (SEQ ID NO: 1159) | 756 | 763 |
| Z17877_PEA_1_T4 (SEQ ID NO: 1160) | 756 | 763 |
| Z17877_PEA_1_T6 (SEQ ID NO: 1161) | 756 | 763 |
| Z17877_PEA_1_T8 (SEQ ID NO: 1163) | 756 | 763 |

Segment cluster Z17877_PEA_{—}1_node_{—}5 (SEQ ID NO:1179) according to the present invention is supported by 80 libraries. The number of libraries was determined as previously described. This segment can be found in the following transcript(s): Z17877_PEA_{—}1_T0 (SEQ ID NO:1157), Z17877_PEA_{—}1_T2 (SEQ ID NO:1158), Z17877_PEA_{—}1_T3 (SEQ ID NO:1159), Z17877_PEA_{—}1_T6 (SEQ ID NO:1161) and Z17877_PEA_{—}1_T8 (SEQ ID NO:1163). Table 29 below describes the starting and ending position of this segment on each transcript.

TABLE 29
Segment location on transcripts

| | Segment | Segment |
|---|---|---|
| | starting | ending |
| Transcript name | position | position |
| Z17877_PEA_1_T0 (SEQ ID NO: 1157) | 764 | 849 |
| Z17877_PEA_1_T2 (SEQ ID NO: 1158) | 764 | 849 |
| Z17877_PEA_1_T3 (SEQ ID NO: 1159) | 764 | 849 |
| Z17877_PEA_1_T6 (SEQ ID NO: 1161) | 764 | 849 |
| Z17877_PEA_1_T8 (SEQ ID NO: 1163) | 764 | 849 |

Segment cluster Z17877_PEA_{—}1_node_{—}6 (SEQ ID NO:1180) according to the present invention can be found in the following transcript(s): Z17877_PEA_{—}1_T0 (SEQ ID NO:1157), Z17877_PEA_{—}1_T2 (SEQ ID NO:1158), Z17877_PEA_{—}1_T3 (SEQ ID NO:1159), Z17877_PEA_{—}1_T4 (SEQ ID NO:1160), Z17877_PEA_{—}1_T6 (SEQ ID NO:1161) and Z17877_PEA_{—}1_T8 (SEQ ID NO:1163). Table 30 below describes the starting and ending position of this segment on each transcript.

TABLE 30
Segment location on transcripts

| | Segment | |
|---|---|---|
| | starting | Segment |
| Transcript name | position | ending position |
| Z17877_PEA_1_T0 (SEQ ID NO: 1157) | 850 | 861 |
| Z17877_PEA_1_T2 (SEQ ID NO: 1158) | 850 | 861 |
| Z17877_PEA_1_T3 (SEQ ID NO: 1159) | 850 | 861 |
| Z17877_PEA_1_T4 (SEQ ID NO: 1160) | 764 | 775 |
| Z17877_PEA_1_T6 (SEQ ID NO: 1161) | 850 | 861 |
| Z17877_PEA_1_T8 (SEQ ID NO: 1163) | 850 | 861 |

Segment cluster Z17877_PEA_{—}1_node_{—}14 (SEQ ID NO:1181) according to the present invention is supported by 83 libraries. The number of libraries was determined as previously described. This segment can be found in the following transcript(s): Z17877_PEA_{—}1_T0 (SEQ ID NO:1157), Z17877_PEA_{—}1_T2 (SEQ ID NO:1158), Z17877_PEA_{—}1_T3 (SEQ ID NO:1159), Z17877_PEA_{—}1_T4 (SEQ ID NO:1160), Z17877_PEA_{—}1_T6 (SEQ ID NO:1161), Z17877_PEA_{—}1_T7 (SEQ ID NO:1162), Z17877_PEA_{—}1_T8 (SEQ ID NO:1163), Z17877_PEA_{—}1_T11 (SEQ ID NO:1164) and Z17877_PEA_{—}1_T12 (SEQ ID NO:1165). Table 31 below describes the starting and ending position of this segment on each transcript.

TABLE 31
Segment location on transcripts

| | Segment | |
|---|---|---|
| | starting | Segment |
| Transcript name | position | ending position |
| Z17877_PEA_1_T0 (SEQ ID NO: 1157) | 1403 | 1480 |
| Z17877_PEA_1_T2 (SEQ ID NO: 1158) | 3027 | 3104 |
| Z17877_PEA_1_T3 (SEQ ID NO: 1159) | 1403 | 1480 |
| Z17877_PEA_1_T4 (SEQ ID NO: 1160) | 2941 | 3018 |
| Z17877_PEA_1_T6 (SEQ ID NO: 1161) | 1403 | 1480 |
| Z17877_PEA_1_T7 (SEQ ID NO: 1162) | 799 | 876 |
| Z17877_PEA_1_T8 (SEQ ID NO: 1163) | 1403 | 1480 |
| Z17877_PEA_1_T11 (SEQ ID NO: 1164) | 799 | 876 |
| Z17877_PEA_1_T12 (SEQ ID NO: 1165) | 799 | 876 |

Segment cluster Z17877_PEA_{—}1_node_{—}17 (SEQ ID NO:1182) according to the present invention is supported by 11 libraries. The number of libraries was determined as previously described. This segment can be found in the following transcript(s): Z17877_PEA_{—}1_T6 (SEQ ID NO:1161), Z17877_PEA_{—}1_T8 (SEQ ID NO:1163) and Z17877_PEA_{—}1_T12 (SEQ ID NO:1165). Table 32 below describes the starting and ending position of this segment on each transcript.

TABLE 32
Segment location on transcripts

| | Segment | |
|---|---|---|
| | starting | Segment |
| Transcript name | position | ending position |
| Z17877_PEA_1_T6 (SEQ ID NO: 1161) | 3312 | 3338 |
| Z17877_PEA_1_T8 (SEQ ID NO: 1163) | 1963 | 1989 |
| Z17877_PEA_1_T12 (SEQ ID NO: 1165) | 1359 | 1385 |

### Expression of c-myc-P64 mRNA, Initiating from Promoter P0 Z17877 Transcripts which are Detectable by Amplicon as Depicted in Sequence Name Z17877Seg8 (SEQ ID NO: 1375) in Normal and Cancerous Colon Tissues

Expression of c-myc-P64 mRNA, initiating from promoter P0 transcripts detectable by or according to seg8, Z17877seg8 amplicon (SEQ ID NO: 1375) and Z17877seg8 F (SEQ ID NO: 1373) and Z17877seg8 R (SEQ ID NO: 1374) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing panel”), to obtain a value of fold up-regulation for each sample relative to median of the normal PM samples.

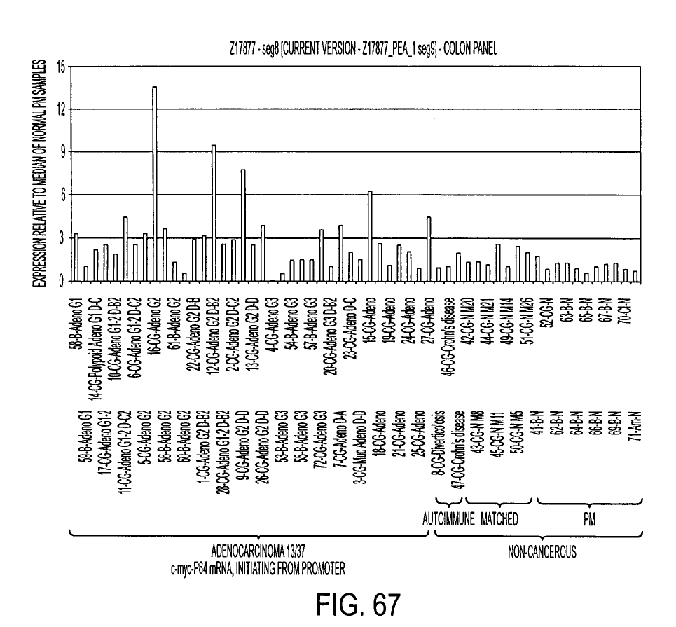

FIG. 67 is a histogram showing over expression of the above-indicated c-myc-P64 mRNA, initiating from promoter P0 transcripts in cancerous colon samples relative to the normal samples. The number and percentage of samples that exhibit at least 3 fold over-expression, out of the total number of samples tested is indicated in the bottom.

As is evident from FIG. 67, the expression of c-myc-P64 mRNA, initiating from promoter P0 transcripts detectable by the above amplicon in cancer samples was significantly higher than in the non-cancerous samples (Sample Nos. 41, 52, 62-67, 69-71 Table 1, “Tissue samples in testing panel”). Notably an over-expression of at least 3 fold was found in 13 out of 37 adenocarcinoma samples,

Statistical analysis was applied to verify the significance of these results, as described below.

The P value for the difference in the expression levels of c-myc-P64 mRNA, initiating from promoter P0 transcripts detectable by the above amplicon in colon cancer samples versus the normal tissue samples was determined by T test as 6.27E-05.

Threshold of 3 fold overexpression was found to differentiate between cancer and normal samples with P value of 1.85E-02 as checked by exact fisher test. The above values demonstrate statistical significance of the results.

Primer pairs are also optionally and preferably encompassed within the present invention; for example, for the above experiment, the following primer pair was used as a non-limiting illustrative example only of a suitable primer pair: Z17877seg8F forward primer (SEQ ID NO: 1373); and Z17877seg8R reverse primer (SEQ ID NO: 1374).

The present invention also preferably encompasses any amplicon obtained through the use of any suitable primer pair; for example, for the above experiment, the following amplicon was obtained as a non-limiting illustrative example only of a suitable amplicon: Z17877seg8 (SEQ ID NO: 1375).

Forward primer (SEQ ID NO: 1373):
AGCAAGGACGCGACTCTCC
Reverse primer (SEQ ID NO: 1374):
AATCCAGCGTCTAAGCAGCTG
Amplicon (SEQ ID NO: 1375):
AGCAAGGACGCGACTCTCCCGACGCGGGGAGGCTATTCTGCCCATTTGGG
GACACTTCCCCGCCGCTGCCAGGACCCGCTTCTCTGAAAGGCTCTCCTTG
CAGCTGCTTAGACGCTGGATT

Combined expression of 19 sequences (T23657seg 29-32 (SEQ ID NO: 1363); T23657seg 22 (SEQ ID NO: 1360); T23657seg 41 (SEQ ID NO: 1366); T23657seg17-18 (SEQ ID NO: 1357); AA315457seg8 (SEQ ID NO:1383); R30650seg76 (SEQ ID NO: 1354); HUM-CEASeg33 (SEQ ID NO: 1345); CEA-Seg35 (SEQ ID NO: 1348); CEA-Seg31 (SEQ ID NO: 1342); AA58339seg1 (SEQ ID NO: 1327); AA583399seg17 (SEQ ID NO: 1324); AA583399-seg30-32 (SEQ ID NO: 1321); HUMCACH1A seg101 (SEQ ID NO: 1337); HSHCGIseg20 (SEQ ID NO: 1378); HSHCGIseg35 (SEQ ID NO: 1381); M78035seg 42 (SEQ ID NO: 1351); T51958seg7 (SEQ ID NO: 1372); T51958seg38 (SEQ ID NO: 1369); Z17877seg8 (SEQ ID NO: 1375)) in normal and cancerous colon tissues.

Expression of solute carrier organic anion transporter family, member 4A1 (SLC04A1), Carcinoembryonic antigen-related cell adhesion molecule 5 [Precursor], myeloma overexpressed gene (in a subset of t(11;14) positive multiple myelomas) (MYEOV), Voltage-dependent L-type calcium channel alpha-1D subunit Calcium channel, L type, alpha-1 polypeptide, isoform 2, TRIM31 tripartite motif, S-adenosylhomocysteine hydrolase (AHCY), Homo sapiens PTK7 protein tyrosine kinase 7 (PTK7) and c-myc-P64 mRNA, initiating from promoter PO transcripts detectable by or according to T23657seg 29-32 (SEQ ID NO: 1363); T23657seg 22 (SEQ ID NO: 1360); T23657seg 41 (SEQ ID NO: 1366); T23657seg17-18 (SEQ ID NO: 1357); AA315457seg8 SEQ ID NO: 1383; R30650seg76 (SEQ ID NO: 1354); HUM-CEASeg33 (SEQ ID NO: 1345); CEA-Seg35 (SEQ ID NO: 1348); CEA-Seg31 (SEQ ID NO: 1342); AA58339seg1 (SEQ ID NO: 1327); AA583399seg17 (SEQ ID NO: 1324); AA583399-seg30-32 (SEQ ID NO: 1321); HUMCACH1A seg101 (SEQ ID NO: 1337); HSHCGIseg20 (SEQ ID NO: 1378); HSHCGIseg35 (SEQ ID NO: 1381); M78035seg 42 (SEQ ID NO: 1351); T51958seg7 (SEQ ID NO: 1372); T51958seg38 (SEQ ID NO: 1369); Z17877seg8 (SEQ ID NO: 1375) amplicons was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicons was normalized to the geometric mean of the quantities of the housekeeping genes. The normalized quantity of each RT sample of each amplicon was then divided by the median of the quantities of the normal post-mortem (PM) samples detected for the same amplicon (Sample Nos. 41, 52, 62-67, 69-71, Table 1, above, “Tissue samples in testing panel”), to obtain a value of fold up-regulation for each sample relative to median of the normal PM samples.

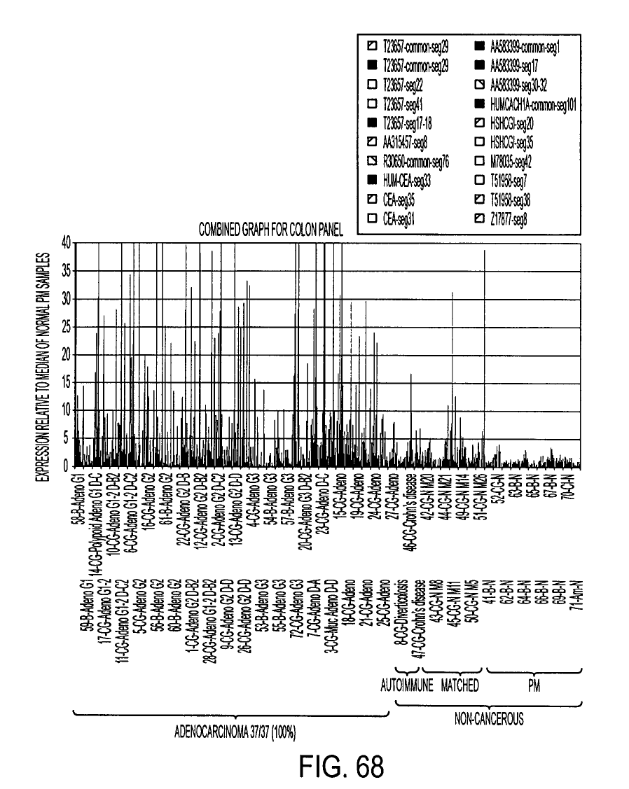

FIG. 68 is a histogram showing over expression of the above-indicated transcripts in cancerous colon samples relative to the normal samples. The number and percentage of samples that exhibit at least 5 fold over-expression of at least one of the sequences, out of the total number of samples tested is indicated in the bottom.

As is evident from FIG. 68, an over-expression of at least 5 fold in at least one of the sequences was found in 37 out of 37 adenocarcinoma samples.

Statistical analysis was applied to verify the significance of these results, as described below. Threshold of 5 fold overexpression of at least one of the amplicons was found to differentiate between cancer and normal samples with P value of 5.31E-10 as checked by exact fisher test.

The above values demonstrate statistical significance of the results.

The FIG. 68 shows combined results for the colon panel marker, as a non-limiting example of a combination of markers according to the present invention.

### Description for Cluster HSHCGI

Cluster HSHCGI features 24 transcript(s) and 29 segment(s) of interest, the names for which are given in Tables 1 and 2, respectively, the sequences themselves are given at the end of the application. The selected protein variants are given in table 3.

TABLE 1
Transcripts of interest

| Transcript Name | SEQ ID NO: |
|---|---|
| HSHCGI_PEA_3_T0 | 1187 |
| HSHCGI_PEA_3_T1 | 1188 |
| HSHCGI_PEA_3_T2 | 1189 |
| HSHCGI_PEA_3_T3 | 1190 |
| HSHCGI_PEA_3_T4 | 1191 |
| HSHCGI_PEA_3_T5 | 1192 |
| HSHCGI_PEA_3_T6 | 1193 |
| HSHCGI_PEA_3_T7 | 1194 |
| HSHCGI_PEA_3_T8 | 1195 |
| HSHCGI_PEA_3_T9 | 1196 |
| HSHCGI_PEA_3_T10 | 1197 |
| HSHCGI_PEA_3_T11 | 1198 |
| HSHCGI_PEA_3_T12 | 1199 |
| HSHCGI_PEA_3_T13 | 1200 |
| HSHCGI_PEA_3_T14 | 1201 |
| HSHCGI_PEA_3_T15 | 1202 |
| HSHCGI_PEA_3_T17 | 1203 |
| HSHCGI_PEA_3_T18 | 1204 |
| HSHCGI_PEA_3_T19 | 1205 |
| HSHCGI_PEA_3_T20 | 1206 |
| HSHCGI_PEA_3_T21 | 1207 |
| HSHCGI_PEA_3_T22 | 1208 |
| HSHCGI_PEA_3_T23 | 1209 |
| HSHCGI_PEA_3_T24 | 1210 |

TABLE 2
Segments of interest

| Segment Name | SEQ ID NO: |
|---|---|
| HSHCGI_PEA_3_node_0 | 1211 |
| HSHCGI_PEA_3_node_2 | 1212 |
| HSHCGI_PEA_3_node_7 | 1213 |
| HSHCGI_PEA_3_node_8 | 1214 |
| HSHCGI_PEA_3_node_14 | 1215 |
| HSHCGI_PEA_3_node_16 | 1216 |
| HSHCGI_PEA_3_node_18 | 1217 |
| HSHCGI_PEA_3_node_20 | 1218 |
| HSHCGI_PEA_3_node_26 | 1219 |
| HSHCGI_PEA_3_node_28 | 1220 |
| HSHCGI_PEA_3_node_30 | 1221 |
| HSHCGI_PEA_3_node_32 | 1222 |
| HSHCGI_PEA_3_node_33 | 1223 |
| HSHCGI_PEA_3_node_34 | 1224 |
| HSHCGI_PEA_3_node_36 | 1225 |
| HSHCGI_PEA_3_node_1 | 1226 |
| HSHCGI_PEA_3_node_4 | 1227 |
| HSHCGI_PEA_3_node_6 | 1228 |
| HSHCGI_PEA_3_node_9 | 1229 |
| HSHCGI_PEA_3_node_11 | 1230 |
| HSHCGI_PEA_3_node_13 | 1231 |
| HSHCGI_PEA_3_node_19 | 1232 |
| HSHCGI_PEA_3_node_21 | 1233 |
| HSHCGI_PEA_3_node_22 | 1234 |
| HSHCGI_PEA_3_node_23 | 1235 |
| HSHCGI_PEA_3_node_24 | 1236 |
| HSHCGI_PEA_3_node_27 | 1237 |
| HSHCGI_PEA_3_node_31 | 1238 |
| HSHCGI_PEA_3_node_35 | 1239 |

TABLE 3
Proteins of interest

| Protein Name | SEQ ID NO: | Corresponding Transcript(s) |
|---|---|---|
| HSHCGI_PEA_3_P17 | 1243 | HSHCGI_PEA_3_T13 (SEQ ID NO: 1200) |
| HSHCGI_PEA_3_P18 | 1244 | HSHCGI_PEA_3_T0 (SEQ ID NO: 1187) |
| HSHCGI_PEA_3_P19 | 1245 | HSHCGI_PEA_3_T11 (SEQ ID NO: 1198) |
| HSHCGI_PEA_3_P1 | 1246 | HSHCGI_PEA_3_T3 (SEQ ID NO: 1190) |
| HSHCGI_PEA_3_P4 | 1247 | HSHCGI_PEA_3_T5 (SEQ ID NO: 1192); HSHCGI_PEA_3_T12 (SEQ |
| | | ID NO: 1199) |
| HSHCGI_PEA_3_P6 | 1248 | HSHCGI_PEA_3_T7 (SEQ ID NO: 1194) |
| HSHCGI_PEA_3_P7 | 1249 | HSHCGI_PEA_3_T8 (SEQ ID NO: 1195) |
| HSHCGI_PEA_3_P8 | 1250 | HSHCGI_PEA_3_T9 (SEQ ID NO: 1196) |
| HSHCGI_PEA_3_P9 | 1251 | HSHCGI_PEA_3_T10 (SEQ ID NO: 1197) |
| HSHCGI_PEA_3_P12 | 1252 | HSHCGI_PEA_3_T14 (SEQ ID NO: 1201); HSHCGI_PEA_3_T15 |
| | | (SEQ ID NO: 1202); HSHCGI_PEA_3_T20 (SEQ ID NO: 1206) |
| HSHCGI_PEA_3_P13 | 1253 | HSHCGI_PEA_3_T17 (SEQ ID NO: 1203); HSHCGI_PEA_3_T19 |
| | | (SEQ ID NO: 1205) |
| HSHCGI_PEA_3_P14 | 1254 | HSHCGI_PEA_3_T18 (SEQ ID NO: 1204) |
| HSHCGI_PEA_3_P15 | 1255 | HSHCGI_PEA_3_T21 (SEQ ID NO: 1207); HSHCGI_PEA_3_T22 |
| | | (SEQ ID NO: 1208) |
| HSHCGI_PEA_3_P16 | 1256 | HSHCGI_PEA_3_T23 (SEQ ID NO: 1209) |
| HSHCGI_PEA_3_P20 | 1257 | HSHCGI_PEA_3_T1 (SEQ ID NO: 1188); HSHCGI_PEA_3_T2 (SEQ |
| | | ID NO: 1189) |
| HSHCGI_PEA_3_P21 | 1258 | HSHCGI_PEA_3_T4 (SEQ ID NO: 1191) |
| HSHCGI_PEA_3_P22 | 1259 | HSHCGI_PEA_3_T6 (SEQ ID NO: 1193) |

As noted above, cluster HSHCGI features 24 transcript(s), which were listed in Table 1 above. A description of each variant protein according to the present invention is now provided.

Variant protein HSHCGI_PEA_{—}3_P17 (SEQ ID NO:1243) according to the present invention has an amino acid sequence as given at the end of the application; it is encoded by transcript(s) HSHCGI_PEA_{—}3_T13 (SEQ ID NO:1200). One or more alignments to one or more previously published protein sequences are given at the end of the application. A brief description of the relationship of the variant protein according to the present invention to each such aligned protein is as follows:

Comparison Report Between HSHCGI_PEA_{—}3_P17 (SEQ ID NO:1243) and TM31_HUMAN (SEQ ID NO: 1242):

1. An isolated chimeric polypeptide encoding for HSHCGI_PEA_{—}3_P17 (SEQ ID NO:1243), comprising a first amino acid sequence being at least 90% homologous to MASGQFVNKLQEEVICPICLDILQKPVTIDCGHNFCPQCITQIGETSCGFFKCPLCKTSVRRDAIRFNSLLRN LVEKIQALQASEVQSKRKEATCPRHQEMFHYFCEDDGKFLCFVCRESKDHKSHNVSLIEEAAQNYQGQIQ EQIQVLQQKEKETVQVKAQGVHRVDVFTDQVEHEKQRILTEFELLHQVLEEEKNFLLSRIYWLGHEGTEA GKHYV corresponding to amino acids 1-218 of TM31_HUMAN (SEQ ID NO:1242), which also corresponds to amino acids 1-218 of HSHCGI_PEA_{—}3_P17 (SEQ ID NO:1243), and a second amino acid sequence being at least 70%, optionally at least 80%, preferably at least 85%, more preferably at least 90% and most preferably at least 95% homologous to a polypeptide having the sequence EIPLMPTVERSQEARCYP (SEQ ID NO:1442) corresponding to amino acids 219-236 of HSHCGI_PEA_{—}3_P17 (SEQ ID NO:1243), wherein said first amino acid sequence and second amino acid sequence are contiguous and in a sequential order.

2. An isolated polypeptide encoding for a tail of HSHCGI_PEA_{—}3_P17 (SEQ ID NO:1243), comprising a polypeptide being at least 70%, optionally at least about 80%, preferably at least about 85%, more preferably at least about 90% and most preferably at least about 95% homologous to the sequence EIPLMPTVERSQEARCYP (SEQ ID NO:1442) in HSHCGI_PEA_{—}3_P17 (SEQ ID NO:1243).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P17 (SEQ ID NO:1243) also has the following non-silent SNPs (Single Nucleotide Polymorphisms) as listed in Table 4, (given according to their position(s) on the amino acid sequence, with the alternative amino acid(s) listed; the last column indicates whether the SNP is known or not; the presence of known SNPs in variant protein HSHCGI_PEA_{—}3_P17 (SEQ ID NO:1243) sequence provides support for the deduced sequence of this variant protein according to the present invention).

TABLE 4
Amino acid mutations

| SNP position(s) on amino | Alternative | Previously |
|---|---|---|
| acid sequence | amino acid(s) | known SNP? |
| 38 | Q -> | No |
| 38 | Q -> K | No |
| 61 | R -> | No |
| 118 | R -> C | Yes |
| 233 | R -> H | Yes |

Variant protein HSHCGI_PEA_{—}3_P17 (SEQ ID NO:1243) is encoded by the following transcript(s): HSHCGI_PEA_{—}3_T13 (SEQ ID NO:1200), for which the sequence(s) is/are given at the end of the application. The coding portion of transcript HSHCGI_PEA_{—}3_T13 (SEQ ID NO:1200) is shown in bold; this coding portion starts at position 111 and ends at position 814. The transcript also has the following SNPs as listed in Table 5 (given according to their position on the nucleotide sequence, with the alternative nucleic acid listed; the last column indicates whether the SNP is known or not; the presence of known SNPs in variant protein HSHCGI_PEA_{—}3_P17 (SEQ ID NO:1243) sequence provides support for the deduced sequence of this variant protein according to the present invention).

TABLE 5
Nucleic acid SNPs

| SNP position on | Alternative | Previously |
|---|---|---|
| nucleotide sequence | nucleic acid | known SNP? |
| 24 | -> C | No |
| 60 | A -> | No |
| 61 | C -> | No |
| 197 | C -> T | Yes |
| 222 | A -> | No |
| 292 | G -> | No |
| 297 | -> G | No |
| 419 | C -> T | Yes |
| 462 | C -> T | Yes |
| 749 | G -> A | Yes |
| 804 | G -> A | Yes |
| 879 | A -> | No |
| 960 | C -> T | Yes |
| 981 | G -> A | Yes |
| 1274 | C -> A | No |
| 1372 | G -> A | Yes |
| 1423 | A -> C | Yes |
| 1592 | G -> A | Yes |
| 1765 | G -> A | Yes |
| 1770 | G -> C | Yes |
| 1858 | T -> C | No |
| 2006 | A -> G | Yes |

Variant protein HSHCGI_PEA_{—}3_P18 (SEQ ID NO:1244) according to the present invention has an amino acid sequence as given at the end of the application; it is encoded by transcript(s) HSHCGI_PEA_{—}3_T0 (SEQ ID NO:1187). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P18 (SEQ ID NO:1244) also has the following non-silent SNPs (Single Nucleotide Polymorphisms) as listed in Table 6, (given according to their position(s) on the amino acid sequence, with the alternative amino acid(s) listed; the last column indicates whether the SNP is known or not; the presence of known SNPs in variant protein HSHCGI_PEA_{—}3_P18 (SEQ ID NO:1244) sequence provides support for the deduced sequence of this variant protein according to the present invention).

TABLE 6
Amino acid mutations

| SNP position(s) on amino | Alternative | Previously |
|---|---|---|
| acid sequence | amino acid(s) | known SNP? |
| 38 | K -> | No |
| 61 | R -> | No |
| 118 | R -> C | Yes |
| 232 | V -> I | Yes |
| 256 | R -> | No |
| 388 | A -> E | No |
| 421 | E -> K | Yes |

Variant protein HSHCGI_PEA_{—}3_P18 (SEQ ID NO:1244) is encoded by the following transcript(s): HSHCGI_PEA_{—}3_T0 (SEQ ID NO:1187), for which the sequence(s) is/are given at the end of the application. The coding portion of transcript HSHCGI_PEA_{—}3_T0 (SEQ ID NO:1187) is shown in bold; this coding portion starts at position 111 and ends at position 1385. The transcript also has the following SNPs as listed in Table 7 (given according to their position on the nucleotide sequence, with the alternative nucleic acid listed; the last column indicates whether the SNP is known or not; the presence of known SNPs in variant protein HSHCGI_PEA_{—}3_P18 (SEQ ID NO:1244) sequence provides support for the deduced sequence of this variant protein according to the present invention).

TABLE 7
Nucleic acid SNPs

| SNP position on | Alternative | Previously |
|---|---|---|
| nucleotide sequence | nucleic acid | known SNP? |
| 24 | -> C | No |
| 60 | A -> | No |
| 61 | C -> | No |
| 197 | C -> T | Yes |
| 222 | A -> | No |
| 292 | G -> | No |
| 297 | -> G | No |
| 419 | C -> T | Yes |
| 462 | C -> T | Yes |
| 749 | G -> A | Yes |
| 804 | G -> A | Yes |
| 878 | A -> | No |
| 959 | C -> T | Yes |
| 980 | G -> A | Yes |
| 1273 | C -> A | No |
| 1371 | G -> A | Yes |
| 1422 | A -> C | Yes |
| 1591 | G -> A | Yes |
| 1764 | G -> A | Yes |
| 1769 | G -> C | Yes |
| 1857 | T -> C | No |
| 2005 | A -> G | Yes |

Variant protein HSHCGI_PEA_{—}3_P19 (SEQ ID NO:1245) according to the present invention has an amino acid sequence as given at the end of the application; it is encoded by transcript(s) HSHCGI_PEA_{—}3_T11 (SEQ ID NO:1198). One or more alignments to one or more previously published protein sequences are given at the end of the application. A brief description of the relationship of the variant protein according to the present invention to each such aligned protein is as follows:

Comparison Report Between HSHCGI_PEA_{—}3_P19 (SEQ ID NO:1245) and TM31_HUMAN_V2 (SEQ ID NO:1241):

1. An isolated chimeric polypeptide encoding for HSHCGI_PEA_{—}3_P19 (SEQ ID NO:1245), comprising a first amino acid sequence being at least 90% homologous to MASGQFVNKLQEEVICPICLDILQKPVTIDCGHNFCLKCITQIGETSCGFFKCPLCKTSVRRDAIRFNSLLRN LVEKIQALQASEVQSKRKEATCPRHQEMFHYFCEDDGKFLCFVCRESKDHKSHNVSLIEEAAQNYQGQIQ EQIQVLQQKEKETVQVKAQGVHRVDVFTDQVEHEKQRILTEFELLHQVLEEEKNFLLSRIYWLGHEGTEA GKHYVASTEPQLNDLKKLVDSLKTKQNMPPRQLLE corresponding to amino acids 1-248 of TM31_HUMAN_V2 (SEQ ID NO:1241), which also corresponds to amino acids 1-248 of HSHCGI_PEA_{—}3_P19 (SEQ ID NO:1245), and a second amino acid sequence being at least 70%, optionally at least 80%, preferably at least 85%, more preferably at least 90% and most preferably at least 95% homologous to a polypeptide having the sequence NWRKNSVKQNQDTTPSQGA (SEQ ID NO:1443) corresponding to amino acids 249-267 of HSHCGI_PEA_{—}3_P19 (SEQ ID NO:1245), wherein said first amino acid sequence and second amino acid sequence are contiguous and in a sequential order.

2. An isolated polypeptide encoding for a tail of HSHCGI_PEA_{—}3_P19 (SEQ ID NO:1245), comprising a polypeptide being at least 70%, optionally at least about 80%, preferably at least about 85%, more preferably at least about 90% and most preferably at least about 95% homologous to the sequence NWRKNSVKQNQDTTPSQGA (SEQ ID NO:1443) in HSHCGI_PEA_{—}3_P19 (SEQ ID NO:1245).

It should be noted that the known protein sequence (TM31_HUMAN (SEQ ID NO:1242)) has one or more changes than the sequence given at the end of the application and named as being the amino acid sequence for TM31_HUMAN_V2 (SEQ ID NO:1241). These changes were previously known to occur and are listed in the table below.

TABLE 8
Changes to TM31_HUMAN_V2 (SEQ ID NO: 1241)

| SNP position(s) on amino | |
|---|---|
| acid sequence | Type of change |
| 38 | conflict |

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P19 (SEQ ID NO:1245) also has the following non-silent SNPs (Single Nucleotide Polymorphisms) as listed in Table 9, (given according to their position(s) on the amino acid sequence, with the alternative amino acid(s) listed; the last column indicates whether the SNP is known or not; the presence of known SNPs in variant protein HSHCGI_PEA_{—}3_P19 (SEQ ID NO:1245) sequence provides support for the deduced sequence of this variant protein according to the present invention).

TABLE 9
Amino acid mutations

| SNP position(s) on amino | Alternative | Previously |
|---|---|---|
| acid sequence | amino acid(s) | known SNP? |
| 38 | K -> | No |
| 61 | R -> | No |
| 118 | R -> C | Yes |
| 232 | V -> I | Yes |
| 261 | T -> M | Yes |

Variant protein HSHCGI_PEA_{—}3_P19 (SEQ ID NO:1245) is encoded by the following transcript(s): HSHCGI_PEA_{—}3_T11 (SEQ ID NO:1198), for which the sequence(s) is/are given at the end of the application. The coding portion of transcript HSHCGI_PEA_{—}3_T11 (SEQ ID NO:1198) is shown in bold; this coding portion starts at position 111 and ends at position 911. The transcript also has the following SNPs as listed in Table 10 (given according to their position on the nucleotide sequence, with the alternative nucleic acid listed; the last column indicates whether the SNP is known or not; the presence of known SNPs in variant protein HSHCGI_PEA_{—}3_P19 (SEQ ID NO:1245) sequence provides support for the deduced sequence of this variant protein according to the present invention).

TABLE 10
Nucleic acid SNPs

| SNP position on | Alternative | Previously |
|---|---|---|
| nucleotide sequence | nucleic acid | known SNP? |
| 24 | -> C | No |
| 60 | A -> | No |
| 61 | C -> | No |
| 197 | C -> T | Yes |
| 222 | A -> | No |
| 292 | G -> | No |
| 297 | -> G | No |
| 419 | C -> T | Yes |
| 462 | C -> T | Yes |
| 749 | G -> A | Yes |
| 804 | G -> A | Yes |
| 892 | C -> T | Yes |
| 913 | G -> A | Yes |
| 1206 | C -> A | No |
| 1304 | G -> A | Yes |
| 1355 | A -> C | Yes |
| 1524 | G -> A | Yes |
| 1697 | G -> A | Yes |
| 1702 | G -> C | Yes |
| 1790 | T -> C | No |
| 1938 | A -> G | Yes |

Variant protein HSHCGI_PEA_{—}3_P1 (SEQ ID NO:1246) according to the present invention has an amino acid sequence as given at the end of the application; it is encoded by transcript(s) HSHCGI_PEA_{—}3_T3 (SEQ ID NO:1190). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P1 (SEQ ID NO:1246) also has the following non-silent SNPs (Single Nucleotide Polymorphisms) as listed in Table 11, (given according to their position(s) on the amino acid sequence, with the alternative amino acid(s) listed; the last column indicates whether the SNP is known or not; the presence of known SNPs in variant protein HSHCGI_PEA_{—}3_P1 (SEQ ID NO:1246) sequence provides support for the deduced sequence of this variant protein according to the present invention).

TABLE 11
Amino acid mutations

| SNP position(s) on amino | Alternative | Previously |
|---|---|---|
| acid sequence | amino acid(s) | known SNP? |
| 38 | K -> | No |
| 61 | R -> | No |
| 118 | R -> C | Yes |
| 232 | V -> I | Yes |
| 256 | R -> | No |
| 388 | A -> E | No |
| 421 | E -> K | Yes |

Variant protein HSHCGI_PEA_{—}3_P1 (SEQ ID NO:1246) is encoded by the following transcript(s): HSHCGI_PEA_{—}3_T3 (SEQ ID NO:1190), for which the sequence(s) is/are given at the end of the application. The coding portion of transcript HSHCGI_PEA_{—}3_T3 (SEQ ID NO:1190) is shown in bold; this coding portion starts at position 139 and ends at position 1413. The transcript also has the following SNPs as listed in Table 12 (given according to their position on the nucleotide sequence, with the alternative nucleic acid listed; the last column indicates whether the SNP is known or not; the presence of known SNPs in variant protein HSHCGI_PEA_{—}3_P1 (SEQ ID NO:1246) sequence provides support for the deduced sequence of this variant protein according to the present invention).

TABLE 12
Nucleic acid SNPs

| SNP position on | Alternative | Previously |
|---|---|---|
| nucleotide sequence | nucleic acid | known SNP? |
| 88 | A -> | No |
| 89 | C -> | No |
| 225 | C -> T | Yes |
| 250 | A -> | No |
| 320 | G -> | No |
| 325 | -> G | No |
| 447 | C -> T | Yes |
| 490 | C -> T | Yes |
| 777 | G -> A | Yes |
| 832 | G -> A | Yes |
| 906 | A -> | No |
| 987 | C -> T | Yes |
| 1008 | G -> A | Yes |
| 1301 | C -> A | No |
| 1399 | G -> A | Yes |
| 1450 | A -> C | Yes |
| 1566 | G -> A | Yes |
| 1571 | G -> C | Yes |
| 1659 | T -> C | No |
| 1807 | A -> G | Yes |

Variant protein HSHCGI_PEA_{—}3_P4 (SEQ ID NO:1247) according to the present invention has an amino acid sequence as given at the end of the application; it is encoded by transcript(s) HSHCGI_PEA_{—}3_T5 (SEQ ID NO:1192). One or more alignments to one or more previously published protein sequences are given at the end of the application. A brief description of the relationship of the variant protein according to the present invention to each such aligned protein is as follows:

Comparison Report Between HSHCGI_PEA_{—}3_P4 (SEQ ID NO:1247) and TM31_HUMAN_V1 (SEQ ID NO:1240):

1. An isolated chimeric polypeptide encoding for HSHCGI_PEA_{—}3_P4 (SEQ ID NO:1247), comprising a first amino acid sequence being at least 90% homologous to MASGQFVNKLQEEVICPICLDILQKPVTIDCGHNFCLKCITQIGETSCGFFKCPLCKTSVRKNAIRFNSLLRN LVEKIQALQASEVQSKRKEATCPRHQEMFHYFCEDDGKFLCFVCRESKDHKSHNVSLIEEAAQNYQGQIQ EQIQVLQQKEKETVQVKAQGVHRVDVFTDQVEHEKQRILTEFELLHQVLEEEKNFLLSRIYWLGHEGTEA GKHYVASTEPQLNDLKKLVDSLKTKQNMPPRQLLEDIKVVLCR corresponding to amino acids 1-256 of TM31_HUMAN_V1 (SEQ ID NO:1240), which also corresponds to amino acids 1-256 of HSHCGI_PEA_{—}3_P4 (SEQ ID NO:1247), and a second amino acid sequence being at least 70%, optionally at least 80%, preferably at least 85%, more preferably at least 90% and most preferably at least 95% homologous to a polypeptide having the sequence YDGPPQMYFAY (SEQ ID NO:1444) corresponding to amino acids 257-267 of HSHCGI_PEA_{—}3_P4 (SEQ ID NO:1247), wherein said first amino acid sequence and second amino acid sequence are contiguous and in a sequential order.

2. An isolated polypeptide encoding for a tail of HSHCGI_PEA_{—}3_P4 (SEQ ID NO:1247), comprising a polypeptide being at least 70%, optionally at least about 80%, preferably at least about 85%, more preferably at least about 90% and most preferably at least about 95% homologous to the sequence YDGPPQMYFAY (SEQ ID NO:1444) in HSHCGI_PEA_{—}3_P4 (SEQ ID NO:1247).

It should be noted that the known protein sequence (TM31_HUMAN (SEQ ID NO:1242)) has one or more changes than the sequence given at the end of the application and named as being the amino acid sequence for TM31_HUMAN_V1 (SEQ ID NO:1240). These changes were previously known to occur and are listed in the table below.

TABLE 13
Changes to TM31_HUMAN_V1 (SEQ ID NO: 1240)

| SNP position(s) on amino | |
|---|---|
| acid sequence | Type of change |
| 38 | conflict |
| 63 | conflict |

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P4 (SEQ ID NO:1247) also has the following non-silent SNPs (Single Nucleotide Polymorphisms) as listed in Table 14, (given according to their position(s) on the amino acid sequence, with the alternative amino acid(s) listed; the last column indicates whether the SNP is known or not; the presence of known SNPs in variant protein HSHCGI_PEA_{—}3_P4 (SEQ ID NO:1247) sequence provides support for the deduced sequence of this variant protein according to the present invention).

TABLE 14
Amino acid mutations

| SNP position(s) on amino acid | Alternative | |
|---|---|---|
| sequence | amino acid(s) | Previously known SNP? |
| 38 | K -> | No |
| 61 | R -> | No |
| 118 | R -> C | Yes |
| 232 | V -> I | Yes |

Variant protein HSHCGI_PEA_{—}3_P4 (SEQ ID NO:1247) is encoded by the following transcript(s): HSHCGI_PEA_{—}3_T5 (SEQ ID NO:1192), for which the sequence(s) is/are given at the end of the application. The coding portion of transcript HSHCGI_PEA_{—}3_T5 (SEQ ID NO:1192) is shown in bold; this coding portion starts at position 139 and ends at position 939. The transcript also has the following SNPs as listed in Table 15 (given according to their position on the nucleotide sequence, with the alternative nucleic acid listed; the last column indicates whether the SNP is known or not; the presence of known SNPs in variant protein HSHCGI_PEA_{—}3_P4 (SEQ ID NO:1247) sequence provides support for the deduced sequence of this variant protein according to the present invention).

TABLE 15
Nucleic acid SNPs

| SNP position on nucleotide | Alternative | Previously |
|---|---|---|
| sequence | nucleic acid | known SNP? |
| 88 | A -> | No |
| 89 | C -> | No |
| 225 | C -> T | Yes |
| 250 | A -> | No |
| 320 | G -> | No |
| 325 | -> G | No |
| 447 | C -> T | Yes |
| 490 | C -> T | Yes |
| 777 | G -> A | Yes |
| 832 | G -> A | Yes |
| 953 | T -> C | Yes |
| 980 | A -> G | Yes |
| 1048 | T -> C | Yes |
| 1072 | T -> C | Yes |
| 1078 | C -> A | Yes |
| 1282 | T -> C | Yes |
| 1283 | G -> T | Yes |
| 1349 | C -> T | Yes |
| 1486 | G -> A | Yes |
| 1541 | G -> A | Yes |
| 1587 | G -> A | Yes |
| 1598 | G -> A | Yes |
| 1635 | G -> T | Yes |
| 1640 | C -> T | Yes |
| 1686 | C -> G | Yes |
| 1688 | A -> G | Yes |
| 1759 | A -> | No |
| 1840 | C -> T | Yes |
| 1861 | G -> A | Yes |
| 2154 | C -> A | No |
| 2252 | G -> A | Yes |
| 2303 | A -> C | Yes |
| 2472 | G -> A | Yes |
| 2645 | G -> A | Yes |
| 2650 | G -> C | Yes |
| 2738 | T -> C | No |
| 2886 | A -> G | Yes |

Variant protein HSHCGI_PEA_{—}3_P6 (SEQ ID NO:1248) according to the present invention has an amino acid sequence as given at the end of the application; it is encoded by transcript(s) HSHCGI_PEA_{—}3_T7 (SEQ ID NO:1194). One or more alignments to one or more previously published protein sequences are given at the end of the application. A brief description of the relationship of the variant protein according to the present invention to each such aligned protein is as follows:

Comparison Report Between HSHCGI_PEA_{—}3_P6 (SEQ ID NO:1248) and TM31_HUMAN_V1 (SEQ ID NO:1240):

1. An isolated chimeric polypeptide encoding for HSHCGI_PEA_{—}3_P6 (SEQ ID NO:1248), comprising a first amino acid sequence being at least 90% homologous to MASGQFVNKLQEEVICPICLDILQKPVTIDCGHNFCLKCITQIGETSCGFFKCPLCKTSVRKNAIRFNSLLRN LVEKIQALQASEVQSKRKEATCPRHQEMFHYFCEDDGKFLCFVCRESKDHKSHNVSLIEEAAQNYQGQIQ EQIQVLQQKEKETVQVKAQGVHRVDVFTDQVEHEKQRILTEFELLHQVLEEEKNFLLSRIYWLGHEGTEA GKHYVASTEPQLNDLKKLVDSLKTKQNMPPRQLLEDIKVVLCR corresponding to amino acids 1-256 of TM31_HUMAN_V1 (SEQ ID NO:1240), which also corresponds to amino acids 1-256 of HSHCGI_PEA_{—}3_P6 (SEQ ID NO:1248), and a second amino acid sequence being at least 70%, optionally at least 80%, preferably at least 85%, more preferably at least 90% and most preferably at least 95% homologous to a polypeptide having the sequence PTPG (SEQ ID NO:1445) corresponding to amino acids 257-260 of HSHCGI_PEA_{—}3_P6 (SEQ ID NO:1248), wherein said first amino acid sequence and second amino acid sequence are contiguous and in a sequential order.

2. An isolated polypeptide encoding for a tail of HSHCGI_PEA_{—}3_P6 (SEQ ID NO:1248), comprising a polypeptide being at least 70%, optionally at least about 80%, preferably at least about 85%, more preferably at least about 90% and most preferably at least about 95% homologous to the sequence PTPG (SEQ ID NO:1445) in HSHCGI_PEA_{—}3_P6 (SEQ ID NO:1248).

It should be noted that the known protein sequence (TM31_HUMAN (SEQ ID NO:1242)) has one or more changes than the sequence given at the end of the application and named as being the amino acid sequence for TM31_HUMAN_V1 (SEQ ID NO:1240). These changes were previously known to occur and are listed in the table below.

TABLE 16
Changes to TM31_HUMAN_V1 (SEQ ID NO: 1240)

| SNP position(s) on amino | |
|---|---|
| acid sequence | Type of change |
| 38 | conflict |
| 63 | conflict |

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P6 (SEQ ID NO:1248) also has the following non-silent SNPs (Single Nucleotide Polymorphisms) as listed in Table 17, (given according to their position(s) on the amino acid sequence, with the alternative amino acid(s) listed; the last column indicates whether the SNP is known or not; the presence of known SNPs in variant protein HSHCGI_PEA_{—}3_P6 (SEQ ID NO:1248) sequence provides support for the deduced sequence of this variant protein according to the present invention).

TABLE 17
Amino acid mutations

| SNP position(s) on amino acid | Alternative | |
|---|---|---|
| sequence | amino acid(s) | Previously known SNP? |
| 38 | K -> | No |
| 61 | R -> | No |
| 118 | R -> C | Yes |
| 232 | V -> I | Yes |

Variant protein HSHCGI_PEA_{—}3_P6 (SEQ ID NO:1248) is encoded by the following transcript(s): HSHCGI_PEA_{—}3_T7 (SEQ ID NO:1194), for which the sequence(s) is/are given at the end of the application. The coding portion of transcript HSHCGI_PEA_{—}3_T7 (SEQ ID NO:1194) is shown in bold; this coding portion starts at position 139 and ends at position 918. The transcript also has the following SNPs as listed in Table 18 (given according to their position on the nucleotide sequence, with the alternative nucleic acid listed; the last column indicates whether the SNP is known or not; the presence of known SNPs in variant protein HSHCGI_PEA_{—}3_P6 (SEQ ID NO:1248) sequence provides support for the deduced sequence of this variant protein according to the present invention).

TABLE 18
Nucleic acid SNPs

| SNP position on nucleotide | | Previously |
|---|---|---|
| sequence | Alternative nucleic acid | known SNP? |
| 88 | A -> | No |
| 89 | C -> | No |
| 225 | C -> T | Yes |
| 250 | A -> | No |
| 320 | G -> | No |
| 325 | -> G | No |
| 447 | C -> T | Yes |
| 490 | C -> T | Yes |
| 777 | G -> A | Yes |
| 832 | G -> A | Yes |
| 1185 | C -> A | No |
| 1283 | G -> A | Yes |
| 1334 | A -> C | Yes |
| 1503 | G -> A | Yes |
| 1676 | G -> A | Yes |
| 1681 | G -> C | Yes |
| 1769 | T -> C | No |
| 1917 | A -> G | Yes |

Variant protein HSHCGI_PEA_{—}3_P7 (SEQ ID NO:1249) according to the present invention has an amino acid sequence as given at the end of the application; it is encoded by transcript(s) HSHCGI_PEA_{—}3_T8 (SEQ ID NO:1195). One or more alignments to one or more previously published protein sequences are given at the end of the application. A brief description of the relationship of the variant protein according to the present invention to each such aligned protein is as follows:

Comparison Report Between HSHCGI_PEA_{—}3_P7 (SEQ ID NO:1249) and TM31_HUMAN_V1 (SEQ ID NO:1240):

1. An isolated chimeric polypeptide encoding for HSHCGI_PEA_{—}3_P7 (SEQ ID NO:1249), comprising a first amino acid sequence being at least 90% homologous to MASGQFVNKLQEEVICPICLDILQKPVTIDCGHNFCLKCITQIGETSCGFFKCPLCKTSVRKNAIRFNSLLRN LVEKIQALQASEVQSKRKEATCPRHQEMFHYFCEDDGKFLCFVCRESKDHKSHNVSLIEEAAQNYQGQIQ EQIQVLQQKEKETVQVKAQGVHRVDVFTDQVEHEKQRILTEFELLHQVLEEEKNFLLSRIYWLGHEGTEA GKHYVASTEPQLNDLKKLVDSLKTKQNMPPRQLLEDIKVVLCRS corresponding to amino acids 1-257 of TM31_HUMAN_V1 (SEQ ID NO:1240), which also corresponds to amino acids 1-257 of HSHCGI_PEA_{—}3_P7 (SEQ ID NO:1249), and a second amino acid sequence being at least 70%, optionally at least 80%, preferably at least 85%, more preferably at least 90% and most preferably at least 95% homologous to a polypeptide having the sequence SFSHTSSPDLTNQLNHIFLEVKSFSFSTQPLFLWNWRKNSVKQNQDTTPSQGA (SEQ ID NO:1446) corresponding to amino acids 258-310 of HSHCGI_PEA_{—}3_P7 (SEQ ID NO:1249), wherein said first amino acid sequence and second amino acid sequence are contiguous and in a sequential order.

2. An isolated polypeptide encoding for a tail of HSHCGI_PEA_{—}3_P7 (SEQ ID NO:1249), comprising a polypeptide being at least 70%, optionally at least about 80%, preferably at least about 85%, more preferably at least about 90% and most preferably at least about 95% homologous to the sequence SFSHTSSPDLTNQLNHIFLEVKSFSFSTQPLFLWNWRKNSVKQNQDTTPSQGA (SEQ ID NO:1446) in HSHCGI_PEA_{—}3_P7 (SEQ ID NO:1249).

It should be noted that the known protein sequence (TM31_HUMAN (SEQ ID NO:1242)) has one or more changes than the sequence given at the end of the application and named as being the amino acid sequence for TM31_HUMAN_V1 (SEQ ID NO:1240). These changes were previously known to occur and are listed in the table below.

TABLE 19
Changes to TM31_HUMAN_V1 (SEQ ID NO: 1240)

| SNP position(s) on amino | |
|---|---|
| acid sequence | Type of change |
| 38 | conflict |
| 63 | conflict |

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P7 (SEQ ID NO:1249) also has the following non-silent SNPs (Single Nucleotide Polymorphisms) as listed in Table 20, (given according to their position(s) on the amino acid sequence, with the alternative amino acid(s) listed; the last column indicates whether the SNP is known or not; the presence of known SNPs in variant protein HSHCGI_PEA_{—}3_P7 (SEQ ID NO:1249) sequence provides support for the deduced sequence of this variant protein according to the present invention).

TABLE 20
Amino acid mutations

| SNP position(s) on amino acid | Alternative | |
|---|---|---|
| sequence | amino acid(s) | Previously known SNP? |
| 38 | K -> | No |
| 61 | R -> | No |
| 118 | R -> C | Yes |
| 232 | V -> I | Yes |
| 277 | E -> | No |
| 304 | T -> M | Yes |

Variant protein HSHCGI_PEA_{—}3_P7 (SEQ ID NO:1249) is encoded by the following transcript(s): HSHCGI_PEA_{—}3_T8 (SEQ ID NO:1195), for which the sequence(s) is/are given at the end of the application. The coding portion of transcript HSHCGI_PEA_{—}3_T8 (SEQ ID NO:1195) is shown in bold; this coding portion starts at position 139 and ends at position 1068. The transcript also has the following SNPs as listed in Table 21 (given according to their position on the nucleotide sequence, with the alternative nucleic acid listed; the last column indicates whether the SNP is known or not; the presence of known SNPs in variant protein HSHCGI_PEA_{—}3_P7 (SEQ ID NO:1249) sequence provides support for the deduced sequence of this variant protein according to the present invention).

TABLE 21
Nucleic acid SNPs

| SNP position on nucleotide | | Previously |
|---|---|---|
| sequence | Alternative nucleic acid | known SNP? |
| 88 | A -> | No |
| 89 | C -> | No |
| 225 | C -> T | Yes |
| 250 | A -> | No |
| 320 | G -> | No |
| 325 | -> G | No |
| 447 | C -> T | Yes |
| 490 | C -> T | Yes |
| 777 | G -> A | Yes |
| 832 | G -> A | Yes |
| 968 | A -> | No |
| 1049 | C -> T | Yes |
| 1070 | G -> A | Yes |
| 1363 | C -> A | No |
| 1461 | G -> A | Yes |
| 1512 | A -> C | Yes |
| 1681 | G -> A | Yes |
| 1854 | G -> A | Yes |
| 1859 | G -> C | Yes |
| 1947 | T -> C | No |
| 2095 | A -> G | Yes |

Variant protein HSHCGI_PEA_{—}3_P8 (SEQ ID NO:1250) according to the present invention has an amino acid sequence as given at the end of the application; it is encoded by transcript(s) HSHCGI_PEA_{—}3_T9 (SEQ ID NO:1196). One or more alignments to one or more previously published protein sequences are given at the end of the application. A brief description of the relationship of the variant protein according to the present invention to each such aligned protein is as follows:

Comparison Report Between HSHCGI_PEA_{—}3_P8 (SEQ ID NO:1250) and TM31_HUMAN_V1 (SEQ ID NO:1240):

1. An isolated chimeric polypeptide encoding for HSHCGI_PEA_{—}3_P8 (SEQ ID NO:1250), comprising a first amino acid sequence being at least 90% homologous to MASGQFVNKLQEEVICPICLDILQKPVTIDCGHNFCLKCITQIGETSCGFFKCPLCKTSVRKNAIRFNSLLRN LVEKIQALQASEVQSKRKEATCPRHQEMFHYFCEDDGKFLCFVCRESKDHKSHNVSLIEEAAQNYQGQIQ EQIQVLQQKEKETVQVKAQGVHRVDVFTDQVEHEKQRILTEFELLHQVLEEEKNFLLSRIYWLGHEGTEA GKHYVASTEPQLNDLKKLVDSLKTKQNMPPRQLLEDIKVVLCRSEEFQFLNPTPVPLELEKKLSEAKSRHD SITGSLKKFKDQLQADRKKDENRFFKSMNKNDMKSWGLLQKNNHKMNKTSEPGSSSAG corresponding to amino acids 1-342 of TM31_HUMAN_V1 (SEQ ID NO:1240), which also corresponds to amino acids 1-342 of HSHCGI_PEA_{—}3_P8 (SEQ ID NO:1250), and a second amino acid sequence being at least 70%, optionally at least 80%, preferably at least 85%, more preferably at least 90% and most preferably at least 95% homologous to a polypeptide having the sequence KSPVSEY corresponding to amino acids 343-349 of HSHCGI_PEA_{—}3_P8 (SEQ ID NO:1250), wherein said first amino acid sequence and second amino acid sequence are contiguous and in a sequential order.

2. An isolated polypeptide encoding for a tail of HSHCGI_PEA_{—}3_P8 (SEQ ID NO:1250), comprising a polypeptide being at least 70%, optionally at least about 80%, preferably at least about 85%, more preferably at least about 90% and most preferably at least about 95% homologous to the sequence KSPVSEY in HSHCGI_PEA_{—}3_P8 (SEQ ID NO:1250).

It should be noted that the known protein sequence (TM31_HUMAN (SEQ ID NO:1242)) has one or more changes than the sequence given at the end of the application and named as being the amino acid sequence for TM31_HUMAN_V1 (SEQ ID NO:1240). These changes were previously known to occur and are listed in the table below.

TABLE 22
Changes to TM31_HUMAN_V1 (SEQ ID NO: 1240)

| SNP position(s) on amino | |
|---|---|
| acid sequence | Type of change |
| 38 | conflict |
| 63 | conflict |

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P8 (SEQ ID NO:1250) also has the following non-silent SNPs (Single Nucleotide Polymorphisms) as listed in Table 23, (given according to their position(s) on the amino acid sequence, with the alternative amino acid(s) listed; the last column indicates whether the SNP is known or not; the presence of known SNPs in variant protein HSHCGI_PEA_{—}3_P8 (SEQ ID NO:1250) sequence provides support for the deduced sequence of this variant protein according to the present invention).

TABLE 23
Amino acid mutations

| SNP position(s) on amino acid | Alternative | |
|---|---|---|
| sequence | amino acid(s) | Previously known SNP? |
| 38 | K -> | No |
| 61 | R -> | No |
| 118 | R -> C | Yes |
| 232 | V -> I | Yes |
| 256 | R -> | No |

Variant protein HSHCGI_PEA_{—}3_P8 (SEQ ID NO:1250) is encoded by the following transcript(s): HSHCGI_PEA_{—}3_T9 (SEQ ID NO:1196), for which the sequence(s) is/are given at the end of the application. The coding portion of transcript HSHCGI_PEA_{—}3_T9 (SEQ ID NO:1196) is shown in bold; this coding portion starts at position 139 and ends at position 1185. The transcript also has the following SNPs as listed in Table 24 (given according to their position on the nucleotide sequence, with the alternative nucleic acid listed; the last column indicates whether the SNP is known or not; the presence of known SNPs in variant protein HSHCGI_PEA_{—}3_P8 (SEQ ID NO:1250) sequence provides support for the deduced sequence of this variant protein according to the present invention).

TABLE 24
Nucleic acid SNPs

| SNP position on nucleotide | | Previously |
|---|---|---|
| sequence | Alternative nucleic acid | know SNP? |
| 88 | A -> | No |
| 89 | C -> | No |
| 225 | C -> T | Yes |
| 250 | A -> | No |
| 320 | G -> | No |
| 325 | -> G | No |
| 447 | C -> T | Yes |
| 490 | C -> T | Yes |
| 777 | G -> A | Yes |
| 832 | G -> A | Yes |
| 906 | A -> | No |
| 987 | C -> T | Yes |
| 1008 | G -> A | Yes |
| 1281 | A -> G | Yes |
| 1613 | C -> A | No |
| 1711 | G -> A | Yes |
| 1762 | A -> C | Yes |
| 1931 | G -> A | Yes |
| 2104 | G -> A | Yes |
| 2109 | G -> C | Yes |
| 2197 | T -> C | No |
| 2345 | A -> G | Yes |

Variant protein HSHCGI_PEA_{—}3_P9 (SEQ ID NO:1251) according to the present invention has an amino acid sequence as given at the end of the application; it is encoded by transcript(s) HSHCGI_PEA_{—}3_T10 (SEQ ID NO:1197). One or more alignments to one or more previously published protein sequences are given at the end of the application. A brief description of the relationship of the variant protein according to the present invention to each such aligned protein is as follows:

Comparison Report Between HSHCGI_PEA_{—}3_P9 (SEQ ID NO:1251) and TM31 HUMAN_V1 (SEQ ID NO:1240):

1. An isolated chimeric polypeptide encoding for HSHCGI_PEA_{—}3_P9 (SEQ ID NO:1251), comprising a first amino acid sequence being at least 90% homologous to MASGQFVNKLQEEVICPICLDILQKPVTIDCGHNFCLKCITQIGETSCGFFKCPLCKTSVRKNAIRFNSLLRN LVEKIQALQASEVQSKRKEATCPRHQEMFHYFCEDDGKFLCFVCRESKDHKSHNVSLIEEAAQNYQGQIQ EQIQVLQQKEKETVQVKAQGVHRVDVFTDQVEHEKQRILTEFELLHQVLEEEKNFLLSRIYWLGHEGTEA GKHYVASTEPQLNDLKKLVDSLKTKQNMPPRQLLEDIKVVLCR corresponding to amino acids 1-256 of TM31_HUMAN_V1 (SEQ ID NO:1240), which also corresponds to amino acids 1-256 of HSHCGI_PEA_{—}3_P9 (SEQ ID NO:1251), and a second amino acid sequence being at least 70%, optionally at least 80%, preferably at least 85%, more preferably at least 90% and most preferably at least 95% homologous to a polypeptide having the sequence TGEKTQ (SEQ ID NO:1448) corresponding to amino acids 257-262 of HSHCGI_PEA_{—}3_P9 (SEQ ID NO:1251), wherein said first amino acid sequence and second amino acid sequence are contiguous and in a sequential order.

2. An isolated polypeptide encoding for a tail of HSHCGI_PEA_{—}3_P9 (SEQ ID NO:1251), comprising a polypeptide being at least 70%, optionally at least about 80%, preferably at least about 85%, more preferably at least about 90% and most preferably at least about 95% homologous to the sequence TGEKTQ (SEQ ID NO:1448) in HSHCGI_PEA_{—}3_P9 (SEQ ID NO:1251).

It should be noted that the known protein sequence (TM31_HUMAN (SEQ ID NO:1242)) has one or more changes than the sequence given at the end of the application and named as being the amino acid sequence for TM31_HUMAN_V1 (SEQ ID NO:1240). These changes were previously known to occur and are listed in the table below.

TABLE 25
Changes to TM31_HUMAN_V1 (SEQ ID NO: 1240)

| SNP position(s) on amino | |
|---|---|
| acid sequence | Type of change |
| 38 | conflict |
| 63 | conflict |

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P9 (SEQ ID NO:1251) also has the following non-silent SNPs (Single Nucleotide Polymorphisms) as listed in Table 26, (given according to their position(s) on the amino acid sequence, with the alternative amino acid(s) listed; the last column indicates whether the SNP is known or not; the presence of known SNPs in variant protein HSHCGI_PEA_{—}3_P9 (SEQ ID NO:1251) sequence provides support for the deduced sequence of this variant protein according to the present invention).

TABLE 26
Amino acid mutations

| SNP position(s) on amino acid | | Previously |
|---|---|---|
| sequence | Alternative amino acid(s) | know SNP? |
| 38 | K -> | No |
| 61 | R -> | No |
| 118 | R -> C | Yes |
| 232 | V -> I | Yes |

Variant protein HSHCGI_PEA_{—}3_P9 (SEQ ID NO:1251) is encoded by the following transcript(s): HSHCGI_PEA_{—}3_T10 (SEQ ID NO:1197), for which the sequence(s) is/are given at the end of the application. The coding portion of transcript HSHCGI_PEA_{—}3_T10 (SEQ ID NO:1197) is shown in bold; this coding portion starts at position 139 and ends at position 924. The transcript also has the following SNPs as listed in Table 27 (given according to their position on the nucleotide sequence, with the alternative nucleic acid listed; the last column indicates whether the SNP is known or not; the presence of known SNPs in variant protein HSHCGI_PEA_{—}3_P9 (SEQ ID NO:1251) sequence provides support for the deduced sequence of this variant protein according to the present invention).

TABLE 27
Nucleic acid SNPs

| SNP position on nucleotide | | Previously |
|---|---|---|
| sequence | Alternative nucleic acid | known SNP? |
| 88 | A -> | No |
| 89 | C -> | No |
| 225 | C -> T | Yes |
| 250 | A -> | No |
| 320 | G -> | No |
| 325 | -> G | No |
| 447 | C -> T | Yes |
| 490 | C -> T | Yes |
| 777 | G -> A | Yes |
| 832 | G -> A | Yes |
| 943 | C -> T | Yes |
| 964 | G -> A | Yes |
| 1257 | C -> A | No |
| 1355 | G -> A | Yes |
| 1406 | A -> C | Yes |
| 1575 | G -> A | Yes |
| 1748 | G -> A | Yes |
| 1753 | G -> C | Yes |
| 1841 | T -> C | No |
| 1989 | A -> G | Yes |

Variant protein HSHCGI_PEA_{—}3_P12 (SEQ ID NO:1252) according to the present invention has an amino acid sequence as given at the end of the application; it is encoded by transcript(s) HSHCGI_PEA_{—}3_T14 (SEQ ID NO:1201). One or more alignments to one or more previously published protein sequences are given at the end of the application. A brief description of the relationship of the variant protein according to the present invention to each such aligned protein is as follows:

Comparison Report Between HSHCGI_PEA_{—}3_P12 (SEQ ID NO:1252) and TM31_HUMAN (SEQ ID NO:1242):

1. An isolated chimeric polypeptide encoding for HSHCGI_PEA_{—}3_P12 (SEQ ID NO:1252), comprising a first amino acid sequence being at least 90% homologous to MNKNDMKSWGLLQKNNHKMNKTSEPGSSSAGGRTTSGPPNHHSSAPSHSLFRASSAGKVTFPVCLLASY DEISGQGASSQDTKTFDVALSEELHAALSEWLTAIRAWFCEVPSS corresponding to amino acids 312-425 of TM31_HUMAN (SEQ ID NO:1242), which also corresponds to amino acids 1-114 of HSHCGI_PEA_{—}3_P12 (SEQ ID NO:1252).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P12 (SEQ ID NO:1252) is encoded by the following transcript(s): HSHCGI_PEA_{—}3_T14 (SEQ ID NO:1201), for which the sequence(s) is/are given at the end of the application. The coding portion of transcript HSHCGI_PEA_{—}3_T14 (SEQ ID NO:1201) is shown in bold; this coding portion starts at position 1795 and ends at position 2136. The transcript also has the following SNPs as listed in Table 28 (given according to their position on the nucleotide sequence, with the alternative nucleic acid listed; the last column indicates whether the SNP is known or not; the presence of known SNPs in variant protein HSHCGI_PEA_{—}3_P12 (SEQ ID NO:1252) sequence provides support for the deduced sequence of this variant protein according to the present invention).

TABLE 28
Nucleic acid SNPs

| SNP position on nucleotide | Alternative | |
|---|---|---|
| sequence | nucleic acid | Previously known SNP? |
| 348 | C -> T | Yes |
| 442 | G -> T | Yes |
| 466 | G -> | Yes |
| 468 | G -> | Yes |
| 823 | T -> C | Yes |
| 850 | A -> G | Yes |
| 918 | T -> C | Yes |
| 942 | T -> C | Yes |
| 948 | C -> A | Yes |
| 1152 | T -> C | Yes |
| 1153 | G -> T | Yes |
| 1219 | C -> T | Yes |
| 1356 | G -> A | Yes |
| 1411 | G -> A | Yes |
| 1457 | G -> A | Yes |
| 1468 | G -> A | Yes |
| 1505 | G -> T | Yes |
| 1510 | C -> T | Yes |
| 1556 | C -> G | Yes |
| 1558 | A -> G | Yes |
| 1629 | A -> | No |
| 1710 | C -> T | Yes |
| 1731 | G -> A | Yes |
| 2024 | C -> A | No |
| 2122 | G -> A | Yes |
| 2173 | A -> C | Yes |
| 2342 | G -> A | Yes |
| 2515 | G -> A | Yes |
| 2520 | G -> C | Yes |
| 2608 | T -> C | No |
| 2756 | A -> G | Yes |

Variant protein HSHCGI_PEA_{—}3_P13 (SEQ ID NO:1253) according to the present invention has an amino acid sequence as given at the end of the application; it is encoded by transcript(s) HSHCGI_PEA_{—}3_T17 (SEQ ID NO:1203). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P13 (SEQ ID NO:1253) is encoded by the following transcript(s): HSHCGI_PEA_{—}3_T17 (SEQ ID NO:1203), for which the sequence(s) is/are given at the end of the application. The coding portion of transcript HSHCGI_PEA_{—}3_T17 (SEQ ID NO:1203) is shown in bold; this coding portion starts at position 585 and ends at position 914. The transcript also has the following SNPs as listed in Table 29 (given according to their position on the nucleotide sequence, with the alternative nucleic acid listed; the last column indicates whether the SNP is known or not; the presence of known SNPs in variant protein HSHCGI_PEA_{—}3_P13 (SEQ ID NO:1253) sequence provides support for the deduced sequence of this variant protein according to the present invention).

TABLE 29
Nucleic acid SNPs

| SNP position on | | |
|---|---|---|
| nucleotide sequence | Alternative nucleic acid | Previously known SNP? |
| 606 | A -> G | Yes |
| 938 | C -> A | No |
| 1036 | G -> A | Yes |
| 1087 | A -> C | Yes |
| 1256 | G -> A | Yes |
| 1429 | G -> A | Yes |
| 1434 | G -> C | Yes |
| 1522 | T -> C | No |
| 1670 | A -> G | Yes |

Variant protein HSHCGI_PEA_{—}3_P14 (SEQ ID NO:1254) according to the present invention has an amino acid sequence as given at the end of the application; it is encoded by transcript(s) HSHCGI_PEA_{—}3_T18 (SEQ ID NO:1204). One or more alignments to one or more previously published protein sequences are given at the end of the application. A brief description of the relationship of the variant protein according to the present invention to each such aligned protein is as follows:

Comparison Report Between HSHCGI_PEA_{—}3_P14 (SEQ ID NO:1254) and TM31_HUMAN_V1 (SEQ ID NO:1240):

1. An isolated chimeric polypeptide encoding for HSHCGI_PEA_{—}3_P14 (SEQ ID NO:1254), comprising a first amino acid sequence being at least 90% homologous to MASGQFVNKLQEEVICPICLDILQKPVTIDCGHNFCLKCITQIGETSCGFFKCPLCKTSVRKNAIRFNSLLRN LVEKIQALQASEVQSKRKEATCPRHQEMFHYFCEDDGKFLCFVCRESKDHKSHNVSLIEEAAQNYQGQIQ EQIQVLQQKEKETVQVKAQGVHRVDVFTDQVEHEKQRILTEFELLHQVLEEEKNFLLSRIYWLGHEGTEA GKHYVASTEPQLNDLKKLVDSLKTKQNMPPRQLLEDIKVVLCRSEEFQFLNPTPVPLELEKKLSEAKSRHD SITGSLKKFKDQLQADRKKDENRFFKSMNKNDMKS corresponding to amino acids 1-319 of TM31_HUMAN_V1 (SEQ ID NO:1240), which also corresponds to amino acids 1-319 of HSHCGI_PEA_{—}3_P14 (SEQ ID NO:1254), and a second amino acid sequence being at least 70%, optionally at least 80%, preferably at least 85%, more preferably at least 90% and most preferably at least 95% homologous to a polypeptide having the sequence CK corresponding to amino acids 320-321 of HSHCGI_PEA_{—}3_P14 (SEQ ID NO:1254), wherein said first amino acid sequence and second amino acid sequence are contiguous and in a sequential order.

It should be noted that the known protein sequence (TM31_HUMAN (SEQ ID NO:1242)) has one or more changes than the sequence given at the end of the application and named as being the amino acid sequence for TM31_HUMAN_V1 (SEQ ID NO:1240). These changes were previously known to occur and are listed in the table below.

TABLE 30
Changes to TM31_HUMAN_V1 (SEQ ID NO: 1240)

| SNP position(s) on amino | |
|---|---|
| acid sequence | Type of change |
| 38 | conflict |
| 63 | conflict |

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P14 (SEQ ID NO:1254) also has the following non-silent SNPs (Single Nucleotide Polymorphisms) as listed in Table 31, (given according to their position(s) on the amino acid sequence, with the alternative amino acid(s) listed; the last column indicates whether the SNP is known or not; the presence of known SNPs in variant protein HSHCGI_PEA_{—}3_P14 (SEQ ID NO:1254) sequence provides support for the deduced sequence of this variant protein according to the present invention).

TABLE 31
Amino acid mutations

| SNP position(s) on | | |
|---|---|---|
| amino acid sequence | Alternative amino acid(s) | Previously known SNP? |
| 38 | K -> | No |
| 61 | R -> | No |
| 118 | R -> C | Yes |
| 232 | V -> I | Yes |
| 256 | R -> | No |

Variant protein HSHCGI_PEA_{—}3_P14 (SEQ ID NO:1254) is encoded by the following transcript(s): HSHCGI_PEA_{—}3_T18 (SEQ ID NO:1204), for which the sequence(s) is/are given at the end of the application. The coding portion of transcript HSHCGI_PEA_{—}3_T18 (SEQ ID NO:1204) is shown in bold; this coding portion starts at position 139 and ends at position 1101. The transcript also has the following SNPs as listed in Table 32 (given according to their position on the nucleotide sequence, with the alternative nucleic acid listed; the last column indicates whether the SNP is known or not; the presence of known SNPs in variant protein HSHCGI_PEA_{—}3_P14 (SEQ ID NO:1254) sequence provides support for the deduced sequence of this variant protein according to the present invention).

TABLE 32
Nucleic acid SNPs

| SNP position on | | |
|---|---|---|
| nucleotide sequence | Alternative nucleic acid | Previously known SNP? |
| 88 | A -> | No |
| 89 | C -> | No |
| 225 | C -> T | Yes |
| 250 | A -> | No |
| 320 | G -> | No |
| 325 | -> G | No |
| 447 | C -> T | Yes |
| 490 | C -> T | Yes |
| 777 | G -> A | Yes |
| 832 | G -> A | Yes |
| 906 | A -> | No |
| 987 | C -> T | Yes |
| 1008 | G -> A | Yes |
| 1320 | A -> G | Yes |
| 1416 | C -> G | Yes |

Variant protein HSHCGI_PEA_{—}3_P15 (SEQ ID NO:1255) according to the present invention has an amino acid sequence as given at the end of the application; it is encoded by transcript(s) HSHCGI_PEA_{—}3_T21 (SEQ ID NO:1207). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P15 (SEQ ID NO:1255) is encoded by the following transcript(s): HSHCGI_PEA_{—}3_T21 (SEQ ID NO:1207), for which the sequence(s) is/are given at the end of the application. The coding portion of transcript HSHCGI_PEA_{—}3_T21 (SEQ ID NO:1207) is shown in bold; this coding portion starts at position 338 and ends at position 505. The transcript also has the following SNPs as listed in Table 33 (given according to their position on the nucleotide sequence, with the alternative nucleic acid listed; the last column indicates whether the SNP is known or not; the presence of known SNPs in variant protein HSHCGI_PEA_{—}3_P15 (SEQ ID NO:1255) sequence provides support for the deduced sequence of this variant protein according to the present invention).

TABLE 33
Nucleic acid SNPs

| SNP position on | | |
|---|---|---|
| nucleotide sequence | Alternative nucleic acid | Previously known SNP? |
| 15 | A -> G | Yes |
| 361 | A -> G | Yes |
| 377 | C -> T | Yes |
| 429 | C -> A | Yes |
| 606 | G -> A | Yes |
| 902 | T -> C | Yes |
| 1104 | G -> C | Yes |
| 1473 | T -> C | Yes |
| 1853 | A -> G | Yes |
| 2005 | C -> A | Yes |
| 2028 | C -> T | Yes |
| 2080 | A -> G | Yes |

Variant protein HSHCGI_PEA_{—}3_P16 (SEQ ID NO:1256) according to the present invention has an amino acid sequence as given at the end of the application; it is encoded by transcript(s) HSHCGI_PEA_{—}3_T23 (SEQ ID NO:1209). One or more alignments to one or more previously published protein sequences are given at the end of the application. A brief description of the relationship of the variant protein according to the present invention to each such aligned protein is as follows:

Comparison Report Between HSHCGI_PEA_{—}3_P16 (SEQ ID NO:1256) and TM31_HUMAN_V1:

1. An isolated chimeric polypeptide encoding for HSHCGI_PEA_{—}3_P16 (SEQ ID NO:1256), comprising a first amino acid sequence being at least 90% homologous to MASGQFVNKLQEEVICPICLDILQKPVTIDCGHNFCLKCITQIGETSCGFFKCPLCKTSVRKNAIRFNSLLRN LVEKIQALQASEVQSKRKEATCPRHQEMFHYFCEDDGKFLCFVCRESKDHKSHNVSLIEEAAQNYQGQIQ EQIQVLQQKEKETVQVKAQGVHRVDVFT corresponding to amino acids 1-171 of TM31_HUMAN_V1 (SEQ ID NO:1240), which also corresponds to amino acids 1-171 of HSHCGI_PEA_{—}3_P16 (SEQ ID NO:1256), and a second amino acid sequence being at least 70%, optionally at least 80%, preferably at least 85%, more preferably at least 90% and most preferably at least 95% homologous to a polypeptide having the sequence VRKTPSHDLWKQKHLCQSSWNPLLH (SEQ ID NO:1449) corresponding to amino acids 172-196 of HSHCGI_PEA_{—}3_P16 (SEQ ID NO:1256), wherein said first amino acid sequence and second amino acid sequence are contiguous and in a sequential order.

2. An isolated polypeptide encoding for a tail of HSHCGI_PEA_{—}3_P16 (SEQ ID NO:1256), comprising a polypeptide being at least 70%, optionally at least about 80%, preferably at least about 85%, more preferably at least about 90% and most preferably at least about 95% homologous to the sequence VRKTPSHDLWKQKHLCQSSWNPLLH (SEQ ID NO:1449) in HSHCGI_PEA_{—}3_P16 (SEQ ID NO:1256).

It should be noted that the known protein sequence (TM31_HUMAN (SEQ ID NO:1242)) has one or more changes than the sequence given at the end of the application and named as being the amino acid sequence for TM31_HUMAN_V1 (SEQ ID NO:1240). These changes were previously known to occur and are listed in the table below.

TABLE 34
Changes to TM31_HUMAN_V1 (SEQ ID NO: 1240)

| SNP position(s) on amino | |
|---|---|
| acid sequence | Type of change |
| 38 | conflict |
| 63 | conflict |

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P16 (SEQ ID NO:1256) also has the following non-silent SNPs (Single Nucleotide Polymorphisms) as listed in Table 35, (given according to their position(s) on the amino acid sequence, with the alternative amino acid(s) listed; the last column indicates whether the SNP is known or not; the presence of known SNPs in variant protein HSHCGI_PEA_{—}3_P16 (SEQ ID NO:1256) sequence provides support for the deduced sequence of this variant protein according to the present invention).

TABLE 35
Amino acid mutations

| SNP position(s) on | | |
|---|---|---|
| amino acid sequence | Alternative amino acid(s) | Previously known SNP? |
| 38 | K -> | No |
| 61 | R -> | No |
| 118 | R -> C | Yes |

Variant protein HSHCGI_PEA_{—}3_P16 (SEQ ID NO:1256) is encoded by the following transcript(s): HSHCGI_PEA_{—}3_T23 (SEQ ID NO:1209), for which the sequence(s) is/are given at the end of the application. The coding portion of transcript HSHCGI_PEA_{—}3_T23 (SEQ ID NO:1209) is shown in bold; this coding portion starts at position 139 and ends at position 726. The transcript also has the following SNPs as listed in Table 36 (given according to their position on the nucleotide sequence, with the alternative nucleic acid listed; the last column indicates whether the SNP is known or not; the presence of known SNPs in variant protein HSHCGI_PEA_{—}3_P16 (SEQ ID NO:1256) sequence provides support for the deduced sequence of this variant protein according to the present invention).

TABLE 36
Nucleic acid SNPs

| SNP position on | | |
|---|---|---|
| nucleotide sequence | Alternative nucleic acid | Previously known SNP? |
| 88 | A -> | No |
| 89 | C -> | No |
| 225 | C -> T | Yes |
| 250 | A -> | No |
| 320 | G -> | No |
| 325 | -> G | No |
| 447 | C -> T | Yes |
| 490 | C -> T | Yes |
| 769 | T -> G | Yes |
| 947 | G -> A | Yes |
| 1000 | C -> A | Yes |
| 1061 | C -> A | Yes |

Variant protein HSHCGI_PEA_{—}3_P20 (SEQ ID NO:1257) according to the present invention has an amino acid sequence as given at the end of the application; it is encoded by transcript(s) HSHCGI_PEA_{—}3_T1 (SEQ ID NO:1188) and HSHCGI_PEA_{—}3_T2 (SEQ ID NO:1189). The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P20 (SEQ ID NO:1257) also has the following non-silent SNPs (Single Nucleotide Polymorphisms) as listed in Table 37, (given according to their position(s) on the amino acid sequence, with the alternative amino acid(s) listed; the last column indicates whether the SNP is known or not; the presence of known SNPs in variant protein HSHCGI_PEA_{—}3_P20 (SEQ ID NO:1257) sequence provides support for the deduced sequence of this variant protein according to the present invention).

TABLE 37
Amino acid mutations

| SNP position(s) on | | |
|---|---|---|
| amino acid sequence | Alternative amino acid(s) | Previously known SNP? |
| 38 | K -> | No |
| 61 | R -> | No |
| 118 | R -> C | Yes |
| 232 | V -> I | Yes |
| 256 | R -> | No |
| 388 | A -> E | No |
| 421 | E -> K | Yes |

Variant protein HSHCGI_PEA_{—}3_P20 (SEQ ID NO:1257) is encoded by the following transcript(s): HSHCGI_PEA_{—}3_T1 (SEQ ID NO:1188) and HSHCGI_PEA_{—}3_T2 (SEQ ID NO:1189), for which the sequence(s) is/are given at the end of the application.

The coding portion of transcript HSHCGI_PEA_{—}3_T1 (SEQ ID NO:1188) is shown in bold; this coding portion starts at position 139 and ends at position 1413. The transcript also has the following SNPs as listed in Table 38 (given according to their position on the nucleotide sequence, with the alternative nucleic acid listed; the last column indicates whether the SNP is known or not; the presence of known SNPs in variant protein HSHCGI_PEA_{—}3_P20 (SEQ ID NO:1257) sequence provides support for the deduced sequence of this variant protein according to the present invention).

TABLE 38
Nucleic acid SNPs

| SNP position on | | |
|---|---|---|
| nucleotide sequence | Alternative nucleic acid | Previously known SNP? |
| 88 | A -> | No |
| 89 | C -> | No |
| 225 | C -> T | Yes |
| 250 | A -> | No |
| 320 | G -> | No |
| 325 | -> G | No |
| 447 | C -> T | Yes |
| 490 | C -> T | Yes |
| 777 | G -> A | Yes |
| 832 | G -> A | Yes |
| 906 | A -> | No |
| 987 | C -> T | Yes |
| 1008 | G -> A | Yes |
| 1301 | C -> A | No |
| 1399 | G -> A | Yes |
| 1450 | A -> C | Yes |
| 1619 | G -> A | Yes |
| 1792 | G -> A | Yes |
| 1797 | G -> C | Yes |
| 1885 | T -> C | No |
| 2033 | A -> G | Yes |

The coding portion of transcript HSHCGI_PEA_{—}3_T2 (SEQ ID NO:1189) is shown in bold; this coding portion starts at position 112 and ends at position 1386. The transcript also has the following SNPs as listed in Table 39 (given according to their position on the nucleotide sequence, with the alternative nucleic acid listed; the last column indicates whether the SNP is known or not; the presence of known SNPs in variant protein HSHCGI_PEA_{—}3_P20 (SEQ ID NO:1257) sequence provides support for the deduced sequence of this variant protein according to the present invention).

TABLE 39
Nucleic acid SNPs

| SNP position on | | |
|---|---|---|
| nucleotide sequence | Alternative nucleic acid | Previously known SNP? |
| 61 | A -> | No |
| 62 | C -> | No |
| 198 | C -> T | Yes |
| 223 | A -> | No |
| 293 | G -> | No |
| 298 | -> G | No |
| 420 | C -> T | Yes |
| 463 | C -> T | Yes |
| 750 | G -> A | Yes |
| 805 | G -> A | Yes |
| 879 | A -> | No |
| 960 | C -> T | Yes |
| 981 | G -> A | Yes |
| 1274 | C -> A | No |
| 1372 | G -> A | Yes |
| 1423 | A -> C | Yes |
| 1592 | G -> A | Yes |
| 1765 | G -> A | Yes |
| 1770 | G -> C | Yes |
| 1858 | T -> C | No |
| 2006 | A -> G | Yes |

Variant protein HSHCGI_PEA_{—}3_P21 (SEQ ID NO:1258) according to the present invention has an amino acid sequence as given at the end of the application; it is encoded by transcript(s) HSHCGI_PEA_{—}3_T4 (SEQ ID NO:1191). One or more alignments to one or more previously published protein sequences are given at the end of the application. A brief description of the relationship of the variant protein according to the present invention to each such aligned protein is as follows:

Comparison Report Between HSHCGI_PEA_{—}3_P21 (SEQ ID NO:1258) and TM31_HUMAN:

1. An isolated chimeric polypeptide encoding for HSHCGI_PEA_{—}3_P21 (SEQ ID NO:1258), comprising a first amino acid sequence being at least 70%, optionally at least 80%, preferably at least 85%, more preferably at least 90% and most preferably at least 95% homologous to a polypeptide having the sequence MHHSDWGNIMWIFQMSPLQNFRKEERNQ (SEQ ID NO:1450) corresponding to amino acids 1-28 of HSHCGI_PEA_{—}3_P21 (SEQ ID NO:1258), and a second amino acid sequence being at least 90% homologous to FLCFVCRESKDHKSHNVSLIEEAAQNYQGQIQEQIQVLQQKEKETVQVKAQGVHRVDVFTDQVEHEKQR1 LTEFELLHQVLEEEKNFLLSRIYWLGHEGTEAGKHYVASTEPQLNDLKKLVDSLKTKQNMPPRQLLEDIK VVLCRSEEFQFLNPTPVPLELEKKLSEAKSRHDSITGSLKKFKDQLQADRKKDENRFFKSMNKNDMKSWG LLQKNNHKMNKTSEPGSSSAGGRTTSGPPNHHSSAPSHSLFRASSAGKVTFPVCLLASYDEISGQGASSQD TKTFDVALSEELHAALSEWLTAIRAWFCEVPSS corresponding to amino acids 112-425 of TM31_HUMAN (SEQ ID NO:1242), which also corresponds to amino acids 29-342 of HSHCGI_PEA_{—}3_P21 (SEQ ID NO:1258), wherein said first amino acid sequence and second amino acid sequence are contiguous and in a sequential order.

2. An isolated polypeptide encoding for a head of HSHCGI_PEA_{—}3_P21 (SEQ ID NO:1258), comprising a polypeptide being at least 70%, optionally at least about 80%, preferably at least about 85%, more preferably at least about 90% and most preferably at least about 95% homologous to the sequence MHHSDWGNIMWIFQMSPLQNFRKEERNQ (SEQ ID NO:1450) of HSHCGI_PEA_{—}3_P21 (SEQ ID NO:1258).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P21 (SEQ ID NO:1258) also has the following non-silent SNPs (Single Nucleotide Polymorphisms) as listed in Table 40, (given according to their position(s) on the amino acid sequence, with the alternative amino acid(s) listed; the last column indicates whether the SNP is known or not; the presence of known SNPs in variant protein HSHCGI_PEA_{—}3_P21 (SEQ ID NO:1258) sequence provides support for the deduced sequence of this variant protein according to the present invention).

TABLE 40
Amino acid mutations

| SNP position(s) on | | |
|---|---|---|
| amino acid sequence | Alternative amino acid(s) | Previously known SNP? |
| 23 | K -> | No |
| 35 | R -> C | Yes |
| 149 | V -> I | Yes |
| 173 | R -> | No |
| 305 | A -> E | No |
| 338 | E -> K | Yes |

Variant protein HSHCGI_PEA_{—}3_P21 (SEQ ID NO:1258) is encoded by the following transcript(s): HSHCGI_PEA_{—}3_T4 (SEQ ID NO:1191), for which the sequence(s) is/are given at the end of the application. The coding portion of transcript HSHCGI_PEA_{—}3_T4 (SEQ ID NO:1191) is shown in bold; this coding portion starts at position 252 and ends at position 1277. The transcript also has the following SNPs as listed in Table 41 (given according to their position on the nucleotide sequence, with the alternative nucleic acid listed; the last column indicates whether the SNP is known or not; the presence of known SNPs in variant protein HSHCGI_PEA_{—}3_P21 (SEQ ID NO:1258) sequence provides support for the deduced sequence of this variant protein according to the present invention).

TABLE 41
Nucleic acid SNPs

| SNP position on | | |
|---|---|---|
| nucleotide sequence | Alternative nucleic acid | Previously known SNP? |
| 88 | A -> | No |
| 89 | C -> | No |
| 225 | C -> T | Yes |
| 250 | A -> | No |
| 320 | G -> | No |
| 325 | -> G | No |
| 354 | C -> T | Yes |
| 641 | G -> A | Yes |
| 696 | G -> A | Yes |
| 770 | A -> | No |
| 851 | C -> T | Yes |
| 872 | G -> A | Yes |
| 1165 | C -> A | No |
| 1263 | G -> A | Yes |
| 1314 | A -> C | Yes |
| 1483 | G -> A | Yes |
| 1656 | G -> A | Yes |
| 1661 | G -> C | Yes |
| 1749 | T -> C | No |
| 1897 | A -> G | Yes |

Variant protein HSHCGI_PEA_{—}3_P22 (SEQ ID NO:1259) according to the present invention has an amino acid sequence as given at the end of the application; it is encoded by transcript(s) HSHCGI_PEA_{—}3_T6 (SEQ ID NO:1193). One or more alignments to one or more previously published protein sequences are given at the end of the application. A brief description of the relationship of the variant protein according to the present invention to each such aligned protein is as follows:

Comparison Report Between HSHCGI_PEA_{—}3_P22 (SEQ ID NO:1259) and TM31_HUMAN:

1. An isolated chimeric polypeptide encoding for HSHCGI_PEA_{—}3_P22 (SEQ ID NO:1259), comprising a first amino acid sequence being at least 90% homologous to MPPRQLLEDIKVVLCRSEEFQFLNPTPVPLELEKKLSEAKSRHDSITGSLKKFKDQLQADRKKDENRFFKS MNKNDMKSWGLLQKNNHKMNKTSEPGSSSAGGRTTSGPPNHHSSAPSHSLFRASSAGKVTFPVCLLASY DEISGQGASSQDTKTFDVALSEELHAALSEWLTAIRAWFCEVPSS corresponding to amino acids 241-425 of TM31_HUMAN (SEQ ID NO:1242), which also corresponds to amino acids 1-185 of HSHCGI_PEA_{—}3_P22 (SEQ ID NO:1259).

The location of the variant protein was determined according to results from a number of different software programs and analyses, including analyses from SignalP and other specialized programs. The variant protein is believed to be located as follows with regard to the cell: intracellularly. The protein localization is believed to be intracellularly because neither of the trans-membrane region prediction programs predicted a trans-membrane region for this protein. In addition both signal-peptide prediction programs predict that this protein is a non-secreted protein.

Variant protein HSHCGI_PEA_{—}3_P22 (SEQ ID NO:1259) also has the following non-silent SNPs (Single Nucleotide Polymorphisms) as listed in Table 42, (given according to their position(s) on the amino acid sequence, with the alternative amino acid(s) listed; the last column indicates whether the SNP is known or not; the presence of known SNPs in variant protein HSHCGI_PEA_{—}3_P22 (SEQ ID NO:1259) sequence provides support for the deduced sequence of this variant protein according to the present invention).

TABLE 42
Amino acid mutations

| SNP position(s) on | | |
|---|---|---|
| amino acid sequence | Alternative amino acid(s) | Previously known SNP? |
| 16 | R -> | No |
| 148 | A -> E | No |
| 181 | E -> K | Yes |

Variant protein HSHCGI_PEA_{—}3_P22 (SEQ ID NO:1259) is encoded by the following transcript(s): HSHCGI_PEA_{—}3_T6 (SEQ ID NO:1193), for which the sequence(s) is/are given at the end of the application. The coding portion of transcript HSHCGI_PEA_{—}3_T6 (SEQ ID NO:1193) is shown in bold; this coding portion starts at position 413 and ends at position 967. The transcript also has the following SNPs as listed in Table 43 (given according to their position on the nucleotide sequence, with the alternative nucleic acid listed; the last column indicates whether the SNP is known or not; the presence of known SNPs in variant protein HSHCGI_PEA_{—}3_P22 (SEQ ID NO:1259) sequence provides support for the deduced sequence of this variant protein according to the present invention).

TABLE 43
Nucleic acid SNPs

| SNP position on nucleotide | Alternative | |
|---|---|---|
| sequence | nucleic acid | Previously known SNP? |
| 9 | A -> T | Yes |
| 73 | A -> G | Yes |
| 331 | G -> A | Yes |
| 386 | G -> A | Yes |
| 460 | A -> | No |
| 541 | C -> T | Yes |
| 562 | G -> A | Yes |
| 855 | C -> A | No |
| 953 | G -> A | Yes |
| 1004 | A -> C | Yes |
| 1173 | G -> A | Yes |
| 1346 | G -> A | Yes |
| 1351 | G -> C | Yes |
| 1439 | T -> C | No |
| 1587 | A -> G | Yes |

As noted above, cluster HSHCGI features 29 segment(s), which were listed in Table 2 above and for which the sequence(s) are given at the end of the application. These segment(s) are portions of nucleic acid sequence(s) which are described herein separately because they are of particular interest. A description of each segment according to the present invention is now provided.

Segment cluster HSHCGI_PEA_{—}3_node_{—}0 (SEQ ID NO:1211) according to the present invention is supported by 3 libraries. The number of libraries was determined as previously described. This segment can be found in the following transcript(s): HSHCGI_PEA_{—}3_T21 (SEQ ID NO:1207) and HSHCGI_PEA_{—}3_T22 (SEQ ID NO:1208). Table 44 below describes the starting and ending position of this segment on each transcript.

TABLE 44
Segment location on transcripts

| | Segment | Segment |
|---|---|---|
| | starting | ending |
| Transcript name | position | position |
| HSHCGI_PEA_3_T21 (SEQ ID NO: 1207) | 1 | 185 |
| HSHCGI_PEA_3_T22 (SEQ ID NO: 1208) | 1 | 185 |

Segment cluster HSHCGI_PEA_{—}3_node_{—}2 (SEQ ID NO:1212) according to the present invention is supported by 6 libraries. The number of libraries was determined as previously described. This segment can be found in the following transcript(s): HSHCGI_PEA_{—}3_T21 (SEQ ID NO:1207) and HSHCGI_PEA_{—}3_T22 (SEQ ID NO:1208). Table 45 below describes the starting and ending position of this segment on each transcript.

TABLE 45
Segment location on transcripts

| | Segment | Segment |
|---|---|---|
| | starting | ending |
| Transcript name | position | position |
| HSHCGI_PEA_3_T21 (SEQ ID NO: 1207) | 186 | 2264 |
| HSHCGI_PEA_3_T22 (SEQ ID NO: 1208) | 186 | 2030 |

Segment cluster HSHCGI_PEA_{—}3_node_{—}7 (SEQ ID NO:1213) according to the present invention is supported by 27 libraries. The number of libraries was determined as previously described. This segment can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T3 (SEQ ID NO:1190), HSHCGI_PEA_{—}3_T4 (SEQ ID NO:1191), HSHCGI_PEA_{—}3_T5 (SEQ ID NO:1192), HSHCGI_PEA_{—}3_T7 (SEQ ID NO:1194), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T11 (SEQ ID NO:1198), HSHCGI_PEA_{—}3_T12 (SEQ ID NO:1199), HSHCGI_PEA_{—}3_T13 (SEQ ID NO:1200), HSHCGI_PEA_{—}3_T18 (SEQ ID NO:1204) and HSHCGI_PEA_{—}3_T23 (SEQ ID NO:1209). Table 46 below describes the starting and ending position of this segment on each transcript.

TABLE 46
Segment location on transcripts

| | Segment | Segment |
|---|---|---|
| | starting | ending |
| Transcript name | position | position |
| HSHCGI_PEA_3_T0 (SEQ ID NO: 1187) | 28 | 307 |
| HSHCGI_PEA_3_T1 (SEQ ID NO: 1188) | 56 | 335 |
| HSHCGI_PEA_3_T2 (SEQ ID NO: 1189) | 29 | 308 |
| HSHCGI_PEA_3_T3 (SEQ ID NO: 1190) | 56 | 335 |
| HSHCGI_PEA_3_T4 (SEQ ID NO: 1191) | 56 | 335 |
| HSHCGI_PEA_3_T5 (SEQ ID NO: 1192) | 56 | 335 |
| HSHCGI_PEA_3_T7 (SEQ ID NO: 1194) | 56 | 335 |
| HSHCGI_PEA_3_T8 (SEQ ID NO: 1195) | 56 | 335 |
| HSHCGI_PEA_3_T9 (SEQ ID NO: 1196) | 56 | 335 |
| HSHCGI_PEA_3_T10 (SEQ ID NO: 1197) | 56 | 335 |
| HSHCGI_PEA_3_T11 (SEQ ID NO: 1198) | 28 | 307 |
| HSHCGI_PEA_3_T12 (SEQ ID NO: 1199) | 56 | 335 |
| HSHCGI_PEA_3_T13 (SEQ ID NO: 1200) | 28 | 307 |
| HSHCGI_PEA_3_T18 (SEQ ID NO: 1204) | 56 | 335 |
| HSHCGI_PEA_3_T23 (SEQ ID NO: 1209) | 56 | 335 |

Segment cluster HSHCGI_PEA_{—}3_node_{—}8 (SEQ ID NO:1214) according to the present invention is supported by 26 libraries. The number of libraries was determined as previously described. This segment can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T3 (SEQ ID NO:1190), HSHCGI_PEA_{—}3_T5 (SEQ ID NO:1192), HSHCGI_PEA_{—}3_T7 (SEQ ID NO:1194), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T11 (SEQ ID NO:1198), HSHCGI_PEA_{—}3_T12 (SEQ ID NO:1199), HSHCGI_PEA_{—}3_T13 (SEQ ID NO:1200), HSHCGI_PEA_{—}3_T18 (SEQ ID NO:1204) and HSHCGI_PEA_{—}3_T23 (SEQ ID NO:1209). Table 47 below describes the starting and ending position of this segment on each transcript.

TABLE 47
Segment location on transcripts

| | Segment | Segment |
|---|---|---|
| | starting | ending |
| Transcript name | position | position |
| HSHCGI_PEA_3_T0 (SEQ ID NO: 1187) | 308 | 443 |
| HSHCGI_PEA_3_T1 (SEQ ID NO: 1188) | 336 | 471 |
| HSHCGI_PEA_3_T2 (SEQ ID NO: 1189) | 309 | 444 |
| HSHCGI_PEA_3_T3 (SEQ ID NO: 1190) | 336 | 471 |
| HSHCGI_PEA_3_T5 (SEQ ID NO: 1192) | 336 | 471 |
| HSHCGI_PEA_3_T7 (SEQ ID NO: 1194) | 336 | 471 |
| HSHCGI_PEA_3_T8 (SEQ ID NO: 1195) | 336 | 471 |
| HSHCGI_PEA_3_T9 (SEQ ID NO: 1196) | 336 | 471 |
| HSHCGI_PEA_3_T10 (SEQ ID NO: 1197) | 336 | 471 |
| HSHCGI_PEA_3_T11 (SEQ ID NO: 1198) | 308 | 443 |
| HSHCGI_PEA_3_T12 (SEQ ID NO: 1199) | 336 | 471 |
| HSHCGI_PEA_3_T13 (SEQ ID NO: 1200) | 308 | 443 |
| HSHCGI_PEA_3_T18 (SEQ ID NO: 1204) | 336 | 471 |
| HSHCGI_PEA_3_T23 (SEQ ID NO: 1209) | 336 | 471 |

Segment cluster HSHCGI_PEA_{—}3_node_{—}14 (SEQ ID NO:1215) according to the present invention is supported by 2 libraries. The number of libraries was determined as previously described. This segment can be found in the following transcript(s): HSHCGI_PEA_{—}3_T23 (SEQ ID NO:1209). Table 48 below describes the starting and ending position of this segment on each transcript.

TABLE 48
Segment location on transcripts

| | Segment | Segment |
|---|---|---|
| | starting | ending |
| Transcript name | position | position |
| HSHCGI_PEA_3_T23 (SEQ ID NO: 1209) | 652 | 1081 |

Segment cluster HSHCGI_PEA_{—}3_node_{—}16 (SEQ ID NO:1216) according to the present invention is supported by 43 libraries. The number of libraries was determined as previously described. This segment can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T3 (SEQ ID NO:1190), HSHCGI_PEA_{—}3_T4 (SEQ ID NO:1191), HSHCGI_PEA_{—}3_T5 (SEQ ID NO:1192), HSHCGI_PEA_{—}3_T6 (SEQ ID NO:1193), HSHCGI_PEA_{—}3_T7 (SEQ ID NO:1194), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T11 (SEQ ID NO:1198), HSHCGI_PEA_{—}3_T12 (SEQ ID NO:1199), HSHCGI_PEA_{—}3_T13 (SEQ ID NO:1200) and HSHCGI_PEA_{—}3_T18 (SEQ ID NO:1204). Table 49 below describes the starting and ending position of this segment on each transcript.

TABLE 49
Segment location on transcripts

| | Segment | Segment |
|---|---|---|
| | starting | ending |
| Transcript name | position | position |
| HSHCGI_PEA_3_T0 (SEQ ID NO: 1187) | 624 | 854 |
| HSHCGI_PEA_3_T1 (SEQ ID NO: 1188) | 652 | 882 |
| HSHCGI_PEA_3_T2 (SEQ ID NO: 1189) | 625 | 855 |
| HSHCGI_PEA_3_T3 (SEQ ID NO: 1190) | 652 | 882 |
| HSHCGI_PEA_3_T4 (SEQ ID NO: 1191) | 516 | 746 |
| HSHCGI_PEA_3_T5 (SEQ ID NO: 1192) | 652 | 882 |
| HSHCGI_PEA_3_T6 (SEQ ID NO: 1193) | 206 | 436 |
| HSHCGI_PEA_3_T7 (SEQ ID NO: 1194) | 652 | 882 |
| HSHCGI_PEA_3_T8 (SEQ ID NO: 1195) | 652 | 882 |
| HSHCGI_PEA_3_T9 (SEQ ID NO: 1196) | 652 | 882 |
| HSHCGI_PEA_3_T10 (SEQ ID NO: 1197) | 652 | 882 |
| HSHCGI_PEA_3_T11 (SEQ ID NO: 1198) | 624 | 854 |
| HSHCGI_PEA_3_T12 (SEQ ID NO: 1199) | 652 | 882 |
| HSHCGI_PEA_3_T13 (SEQ ID NO: 1200) | 624 | 854 |
| HSHCGI_PEA_3_T18 (SEQ ID NO: 1204) | 652 | 882 |

Segment cluster HSHCGI_PEA_{—}3_node_{—}18 (SEQ ID NO:1217) according to the present invention is supported by 4 libraries. The number of libraries was determined as previously described. This segment can be found in the following transcript(s): HSHCGI_PEA_{—}3_T14 (SEQ ID NO:1201). Table 50 below describes the starting and ending position of this segment on each transcript.

TABLE 50
Segment location on transcripts

| | Segment | Segment |
|---|---|---|
| | starting | ending |
| Transcript name | position | position |
| HSHCGI_PEA_3_T14 (SEQ ID NO: 1201) | 1 | 752 |

Segment cluster HSHCGI_PEA_{—}3_node_{—}20 (SEQ ID NO:1218) according to the present invention is supported by 11 libraries. The number of libraries was determined as previously described. This segment can be found in the following transcript(s): HSHCGI_PEA_{—}3_T5 (SEQ ID NO:1192), HSHCGI_PEA_{—}3_T12 (SEQ ID NO:1199) and HSHCGI_PEA_{—}3_T14 (SEQ ID NO:1201). Table 51 below describes the starting and ending position of this segment on each transcript.

TABLE 51
Segment location on transcripts

| | Segment | Segment |
|---|---|---|
| | starting | ending |
| Transcript name | position | position |
| HSHCGI_PEA_3_T5 (SEQ ID NO: 1192) | 906 | 1696 |
| HSHCGI_PEA_3_T12 (SEQ ID NO: 1199) | 906 | 1696 |
| HSHCGI_PEA_3_T14 (SEQ ID NO: 1201) | 776 | 1566 |

Segment cluster HSHCGI_PEA_{—}3_node_{—}26 (SEQ ID NO:1219) according to the present invention is supported by 5 libraries. The number of libraries was determined as previously described. This segment can be found in the following transcript(s): HSHCGI_PEA_{—}3_T15 (SEQ ID NO:1202), HSHCGI_PEA_{—}3_T19 (SEQ ID NO:1205), HSHCGI_PEA_{—}3_T20 (SEQ ID NO:1206) and HSHCGI_PEA_{—}3_T24 (SEQ ID NO:1210). Table 52 below describes the starting and ending position of this segment on each transcript.

TABLE 52
Segment location on transcripts

| | Segment | Segment |
|---|---|---|
| | starting | ending |
| Transcript name | position | position |
| HSHCGI_PEA_3_T15 (SEQ ID NO: 1202) | 1 | 420 |
| HSHCGI_PEA_3_T19 (SEQ ID NO: 1205) | 1 | 420 |
| HSHCGI_PEA_3_T20 (SEQ ID NO: 1206) | 1 | 420 |
| HSHCGI_PEA_3_T24 (SEQ ID NO: 1210) | 1 | 420 |

Segment cluster HSHCGI_PEA_{—}3_node_{—}28 (SEQ ID NO:1220) according to the present invention is supported by 3 libraries. The number of libraries was determined as previously described. This segment can be found in the following transcript(s): HSHCGI_PEA_{—}3_T18 (SEQ ID NO:1204) and HSHCGI_PEA_{—}3_T24 (SEQ ID NO:1210). Table 53 below describes the starting and ending position of this segment on each transcript.

TABLE 53
Segment location on transcripts

| | Segment | Segment |
|---|---|---|
| | starting | ending |
| Transcript name | position | position |
| HSHCGI_PEA_3_T18 (SEQ ID NO: 1204) | 1097 | 1473 |
| HSHCGI_PEA_3_T24 (SEQ ID NO: 1210) | 496 | 872 |

Segment cluster HSHCGI_PEA_{—}3_node_{—}30 (SEQ ID NO:1221) according to the present invention is supported by 3 libraries. The number of libraries was determined as previously described. This segment can be found in the following transcript(s): HSHCGI_PEA_{—}3_T17 (SEQ ID NO:1203). Table 54 below describes the starting and ending position of this segment on each transcript.

TABLE 54
Segment location on transcripts

| | Segment | Segment |
|---|---|---|
| | starting | ending |
| Transcript name | position | position |
| HSHCGI_PEA_3_T17 (SEQ ID NO: 1203) | 1 | 421 |

Segment cluster HSHCGI_PEA_{—}3_node_{—}32 (SEQ ID NO:1222) according to the present invention is supported by 7 libraries. The number of libraries was determined as previously described. This segment can be found in the following transcript(s): HSHCGI_PEA_{—}3_T9 (SEQ ID NO:1196), HSHCGI_PEA_{—}3_T12 (SEQ ID NO:1199), HSHCGI_PEA_{—}3_T17 (SEQ ID NO:1203) and HSHCGI_PEA_{—}3_T19 (SEQ ID NO:1205). Table 55 below describes the starting and ending position of this segment on each transcript.

TABLE 55
Segment location on transcripts

| | Segment | Segment |
|---|---|---|
| | starting | ending |
| Transcript name | position | position |
| HSHCGI_PEA_3_T9 (SEQ ID NO: 1196) | 1163 | 1474 |
| HSHCGI_PEA_3_T12 (SEQ ID NO: 1199) | 2016 | 2327 |
| HSHCGI_PEA_3_T17 (SEQ ID NO: 1203) | 488 | 799 |
| HSHCGI_PEA_3_T19 (SEQ ID NO: 1205) | 562 | 873 |

Segment cluster HSHCGI_PEA_{—}3_node_{—}33 (SEQ ID NO:1223) according to the present invention is supported by 50 libraries. The number of libraries was determined as previously described. This segment can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T3 (SEQ ID NO:1190), HSHCGI_PEA_{—}3_T4 (SEQ ID NO:1191), HSHCGI_PEA_{—}3_T5 (SEQ ID NO:1192), HSHCGI_PEA_{—}3_T6 (SEQ ID NO:1193), HSHCGI_PEA_{—}3_T7 (SEQ ID NO:1194), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T11 (SEQ ID NO:1198), HSHCGI_PEA_{—}3_T12 (SEQ ID NO:1199), HSHCGI_PEA_{—}3_T13 (SEQ ID NO:1200), HSHCGI_PEA_{—}3_T14 (SEQ ID NO:1201), HSHCGI_PEA_{—}3_T15 (SEQ ID NO:1202), HSHCGI_PEA_{—}3_T17 (SEQ ID NO:1203), HSHCGI_PEA_{—}3_T19 (SEQ ID NO:1205) and HSHCGI_PEA_{—}3_T20 (SEQ ID NO:1206). Table 56 below describes the starting and ending position of this segment on each transcript.

TABLE 56
Segment location on transcripts

| | Segment | Segment |
|---|---|---|
| | starting | ending |
| Transcript name | position | position |
| HSHCGI_PEA_3_T0 (SEQ ID NO: 1187) | 1135 | 1452 |
| HSHCGI_PEA_3_T1 (SEQ ID NO: 1188) | 1163 | 1480 |
| HSHCGI_PEA_3_T2 (SEQ ID NO: 1189) | 1136 | 1453 |
| HSHCGI_PEA_3_T3 (SEQ ID NO: 1190) | 1163 | 1480 |
| HSHCGI_PEA_3_T4 (SEQ ID NO: 1191) | 1027 | 1344 |
| HSHCGI_PEA_3_T5 (SEQ ID NO: 1192) | 2016 | 2333 |
| HSHCGI_PEA_3_T6 (SEQ ID NO: 1193) | 717 | 1034 |
| HSHCGI_PEA_3_T7 (SEQ ID NO: 1194) | 1047 | 1364 |
| HSHCGI_PEA_3_T8 (SEQ ID NO: 1195) | 1225 | 1542 |
| HSHCGI_PEA_3_T9 (SEQ ID NO: 1196) | 1475 | 1792 |
| HSHCGI_PEA_3_T10 (SEQ ID NO: 1197) | 1119 | 1436 |
| HSHCGI_PEA_3_T11 (SEQ ID NO: 1198) | 1068 | 1385 |
| HSHCGI_PEA_3_T12 (SEQ ID NO: 1199) | 2328 | 2645 |
| HSHCGI_PEA_3_T13 (SEQ ID NO: 1200) | 1136 | 1453 |
| HSHCGI_PEA_3_T14 (SEQ ID NO: 1201) | 1886 | 2203 |
| HSHCGI_PEA_3_T15 (SEQ ID NO: 1202) | 562 | 879 |
| HSHCGI_PEA_3_T17 (SEQ ID NO: 1203) | 800 | 1117 |
| HSHCGI_PEA_3_T19 (SEQ ID NO: 1205) | 874 | 1191 |
| HSHCGI_PEA_3_T20 (SEQ ID NO: 1206) | 562 | 879 |

Segment cluster HSHCGI_PEA_{—}3_node_{—}34 (SEQ ID NO:1224) according to the present invention is supported by 32 libraries. The number of libraries was determined as previously described. This segment can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T4 (SEQ ID NO:1191), HSHCGI_PEA_{—}3_T5 (SEQ ID NO:1192), HSHCGI_PEA_{—}3_T6 (SEQ ID NO:1193), HSHCGI_PEA_{—}3_T7 (SEQ ID NO:1194), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T11 (SEQ ID NO:1198), HSHCGI_PEA_{—}3_T12 (SEQ ID NO:1199), HSHCGI_PEA_{—}3_T13 (SEQ ID NO:1200), HSHCGI_PEA_{—}3_T14 (SEQ ID NO:1201), HSHCGI_PEA_{—}3_T15 (SEQ ID NO:1202), HSHCGI_PEA_{—}3_T17 (SEQ ID NO:1203) and HSHCGI_PEA_{—}3_T19 (SEQ ID NO:1205). Table 57 below describes the starting and ending position of this segment on each transcript.

TABLE 57
Segment location on transcripts

| | Segment | Segment |
|---|---|---|
| | starting | ending |
| Transcript name | position | position |
| HSHCGI_PEA_3_T0 (SEQ ID NO: 1187) | 1453 | 1678 |
| HSHCGI_PEA_3_T1 (SEQ ID NO: 1188) | 1481 | 1706 |
| HSHCGI_PEA_3_T2 (SEQ ID NO: 1189) | 1454 | 1679 |
| HSHCGI_PEA_3_T4 (SEQ ID NO: 1191) | 1345 | 1570 |
| HSHCGI_PEA_3_T5 (SEQ ID NO: 1192) | 2334 | 2559 |
| HSHCGI_PEA_3_T6 (SEQ ID NO: 1193) | 1035 | 1260 |
| HSHCGI_PEA_3_T7 (SEQ ID NO: 1194) | 1365 | 1590 |
| HSHCGI_PEA_3_T8 (SEQ ID NO: 1195) | 1543 | 1768 |
| HSHCGI_PEA_3_T9 (SEQ ID NO: 1196) | 1793 | 2018 |
| HSHCGI_PEA_3_T10 (SEQ ID NO: 1197) | 1437 | 1662 |
| HSHCGI_PEA_3_T11 (SEQ ID NO: 1198) | 1386 | 1611 |
| HSHCGI_PEA_3_T12 (SEQ ID NO: 1199) | 2646 | 2871 |
| HSHCGI_PEA_3_T13 (SEQ ID NO: 1200) | 1454 | 1679 |
| HSHCGI_PEA_3_T14 (SEQ ID NO: 1201) | 2204 | 2429 |
| HSHCGI_PEA_3_T15 (SEQ ID NO: 1202) | 880 | 1105 |
| HSHCGI_PEA_3_T17 (SEQ ID NO: 1203) | 1118 | 1343 |
| HSHCGI_PEA_3_T19 (SEQ ID NO: 1205) | 1192 | 1417 |

Segment cluster HSHCGI_PEA_{—}3_node_{—}36 (SEQ ID NO:1225) according to the present invention is supported by 38 libraries. The number of libraries was determined as previously described. This segment can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T3 (SEQ ID NO:1190), HSHCGI_PEA_{—}3_T4 (SEQ ID NO:1191), HSHCGI_PEA_{—}3_T5 (SEQ ID NO:1192), HSHCGI_PEA_{—}3_T6 (SEQ ID NO:1193), HSHCGI_PEA_{—}3_T7 (SEQ ID NO:1194), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T11 (SEQ ID NO:1198), HSHCGI_PEA_{—}3_T12 (SEQ ID NO:1199), HSHCGI_PEA_{—}3_T13 (SEQ ID NO:1200), HSHCGI_PEA_{—}3_T14 (SEQ ID NO:1201), HSHCGI_PEA_{—}3_T15 (SEQ ID NO:1202), HSHCGI_PEA_{—}3_T17 (SEQ ID NO:1203), HSHCGI_PEA_{—}3_T19 (SEQ ID NO:1205) and HSHCGI_PEA_{—}3_T20 (SEQ ID NO:1206). Table 58 below describes the starting and ending position of this segment on each transcript.

TABLE 58
Segment location on transcripts

| | Segment | Segment |
|---|---|---|
| | starting | ending |
| Transcript name | position | position |
| HSHCGI_PEA_3_T0 (SEQ ID NO: 1187) | 1792 | 2038 |
| HSHCGI_PEA_3_T1 (SEQ ID NO: 1188) | 1820 | 2066 |
| HSHCGI_PEA_3_T2 (SEQ ID NO: 1189) | 1793 | 2039 |
| HSHCGI_PEA_3_T3 (SEQ ID NO: 1190) | 1594 | 1840 |
| HSHCGI_PEA_3_T4 (SEQ ID NO: 1191) | 1684 | 1930 |
| HSHCGI_PEA_3_T5 (SEQ ID NO: 1192) | 2673 | 2919 |
| HSHCGI_PEA_3_T6 (SEQ ID NO: 1193) | 1374 | 1620 |
| HSHCGI_PEA_3_T7 (SEQ ID NO: 1194) | 1704 | 1950 |
| HSHCGI_PEA_3_T8 (SEQ ID NO: 1195) | 1882 | 2128 |
| HSHCGI_PEA_3_T9 (SEQ ID NO: 1196) | 2132 | 2378 |
| HSHCGI_PEA_3_T10 (SEQ ID NO: 1197) | 1776 | 2022 |
| HSHCGI_PEA_3_T11 (SEQ ID NO: 1198) | 1725 | 1971 |
| HSHCGI_PEA_3_T12 (SEQ ID NO: 1199) | 2985 | 3231 |
| HSHCGI_PEA_3_T13 (SEQ ID NO: 1200) | 1793 | 2039 |
| HSHCGI_PEA_3_T14 (SEQ ID NO: 1201) | 2543 | 2789 |
| HSHCGI_PEA_3_T15 (SEQ ID NO: 1202) | 1219 | 1465 |
| HSHCGI_PEA_3_T17 (SEQ ID NO: 1203) | 1457 | 1703 |
| HSHCGI_PEA_3_T19 (SEQ ID NO: 1205) | 1531 | 1777 |
| HSHCGI_PEA_3_T20 (SEQ ID NO: 1206) | 993 | 1239 |

According to an optional embodiment of the present invention, short segments related to the above cluster are also provided. These segments are up to about 120 bp in length, and so are included in a separate description.

Segment cluster HSHCGI_PEA_{—}3_node_{—}1 (SEQ ID NO:1226) according to the present invention is supported by 0 libraries. The number of libraries was determined as previously described. This segment can be found in the following transcript(s): HSHCGI_PEA_{—}3_T0 (SEQ ID NO:1187), HSHCGI_PEA_{—}3_T11 (SEQ ID NO:1198) and HSHCGI_PEA_{—}3_T13 (SEQ ID NO:1200). Table 59 below describes the starting and ending position of this segment on each transcript.

TABLE 59
Segment location on transcripts

| | Segment | Segment |
|---|---|---|
| | starting | ending |
| Transcript name | position | position |
| HSHCGI_PEA_3_T0 (SEQ ID NO: 1187) | 1 | 27 |
| HSHCGI_PEA_3_T11 (SEQ ID NO: 1198) | 1 | 27 |
| HSHCGI_PEA_3_T13 (SEQ ID NO: 1200) | 1 | 27 |

Segment cluster HSHCGI_PEA_{—}3_node_{—}4 (SEQ ID NO:1227) according to the present invention is supported by 4 libraries. The number of libraries was determined as previously described. This segment can be found in the following transcript(s): HSHCGI_PEA_{—}3_T2 (SEQ ID NO:1189). Table 60 below describes the starting and ending position of this segment on each transcript.

TABLE 60
.Segment location on transcripts

| | Segment | Segment |
|---|---|---|
| | starting | ending |
| Transcript name | position | position |
| HSHCGI_PEA_3_T2 (SEQ ID NO: 1189) | 1 | 28 |

Segment cluster HSHCGI_PEA_{—}3_node_{—}6 (SEQ ID NO:1228) according to the present invention is supported by 3 libraries. The number of libraries was determined as previously described. This segment can be found in the following transcript(s): HSHCGI_PEA_{—}3_T1 (SEQ ID NO:1188), HSHCGI_PEA_{—}3_T3 (SEQ ID NO:1190), HSHCGI_PEA_{—}3_T4 (SEQ ID NO:1191), HSHCGI_PEA_{—}3_T5 (SEQ ID NO:1192), HSHCGI_PEA_{—}3_T7 (SEQ ID NO:1194), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T12 (SEQ ID NO:1199), HSHCGI_PEA_{—}3_T18 (SEQ ID NO:1204) and HSHCGI_PEA_{—}3_T23 (SEQ ID NO:1209). Table 61 below describes the starting and ending position of this segment on each transcript.

TABLE 61
Segment location on transcripts

| | Segment | Segment |
|---|---|---|
| | starting | ending |
| Transcript name | position | position |
| HSHCGI_PEA_3_T1 (SEQ ID NO: 1188) | 1 | 55 |
| HSHCGI_PEA_3_T3 (SEQ ID NO: 1190) | 1 | 55 |
| HSHCGI_PEA_3_T4 (SEQ ID NO: 1191) | 1 | 55 |
| HSHCGI_PEA_3_T5 (SEQ ID NO: 1192) | 1 | 55 |
| HSHCGI_PEA_3_T7 (SEQ ID NO: 1194) | 1 | 55 |
| HSHCGI_PEA_3_T8 (SEQ ID NO: 1195) | 1 | 55 |
| HSHCGI_PEA_3_T9 (SEQ ID NO: 1196) | 1 | 55 |
| HSHCGI_PEA_3_T10 (SEQ ID NO: 1197) | 1 | 55 |
| HSHCGI_PEA_3_T12 (SEQ ID NO: 1199) | 1 | 55 |
| HSHCGI_PEA_3_T18 (SEQ ID NO: 1204) | 1 | 55 |
| HSHCGI_PEA_3_T23 (SEQ ID NO: 1209) | 1 | 55 |

Segment cluster HSHCGI_PEA_{—}3_node_{—}9 (SEQ ID NO:1229) according to the present invention is supported by 32 libraries. The number of libraries was determined as previously described. This segment can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T3 (SEQ ID NO:1190), HSHCGI_PEA_{—}3_T4 (SEQ ID NO:1191), HSHCGI_PEA_{—}3_T5 (SEQ ID NO:1192), HSHCGI_PEA_{—}3_T7 (SEQ ID NO:1194), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T11 (SEQ ID NO:1198), HSHCGI_PEA_{—}3_T12 (SEQ ID NO:1199), HSHCGI_PEA_{—}3_T13 (SEQ ID NO:1200), HSHCGI_PEA_{—}3_T18 (SEQ ID NO:1204) and HSHCGI_PEA_{—}3_T23 (SEQ ID NO:1209). Table 62 below describes the starting and ending position of this segment on each transcript.

TABLE 62
Segment location on transcripts

| | Segment | Segment |
|---|---|---|
| | starting | ending |
| Transcript name | position | position |
| HSHCGI_PEA_3_T0 (SEQ ID NO: 1187) | 444 | 527 |
| HSHCGI_PEA_3_T1 (SEQ ID NO: 1188) | 472 | 555 |
| HSHCGI_PEA_3_T2 (SEQ ID NO: 1189) | 445 | 528 |
| HSHCGI_PEA_3_T3 (SEQ ID NO: 1190) | 472 | 555 |
| HSHCGI_PEA_3_T4 (SEQ ID NO: 1191) | 336 | 419 |
| HSHCGI_PEA_3_T5 (SEQ ID NO: 1192) | 472 | 555 |
| HSHCGI_PEA_3_T7 (SEQ ID NO: 1194) | 472 | 555 |
| HSHCGI_PEA_3_T8 (SEQ ID NO: 1195) | 472 | 555 |
| HSHCGI_PEA_3_T9 (SEQ ID NO: 1196) | 472 | 555 |
| HSHCGI_PEA_3_T10 (SEQ ID NO: 1197) | 472 | 555 |
| HSHCGI_PEA_3_T11 (SEQ ID NO: 1198) | 444 | 527 |
| HSHCGI_PEA_3_T12 (SEQ ID NO: 1199) | 472 | 555 |
| HSHCGI_PEA_3_T13 (SEQ ID NO: 1200) | 444 | 527 |
| HSHCGI_PEA_3_T18 (SEQ ID NO: 1204) | 472 | 555 |
| HSHCGI_PEA_3_T23 (SEQ ID NO: 1209) | 472 | 555 |

Segment cluster HSHCGI_PEA_{—}3_node_{—}11 (SEQ ID NO:1230) according to the present invention is supported by 1 libraries. The number of libraries was determined as previously described. This segment can be found in the following transcript(s): HSHCGI_PEA_{—}3_T6 (SEQ ID NO:1193). Table 63 below describes the starting and ending position of this segment on each transcript.

TABLE 63
Segment location on transcripts

| | Segment | Segment |
|---|---|---|
| | starting | ending |
| Transcript name | position | position |
| HSHCGI_PEA_3_T6 (SEQ ID NO: 1193) | 1 | 109 |

Segment cluster HSHCGI_PEA_{—}3_node_{—}13 (SEQ ID NO:1231) according to the present invention is supported by 35 libraries. The number of libraries was determined as previously described. This segment can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T3 (SEQ ID NO:1190), HSHCGI_PEA_{—}3_T4 (SEQ ID NO:1191), HSHCGI_PEA_{—}3_T5 (SEQ ID NO:1192), HSHCGI_PEA_{—}3_T6 (SEQ ID NO:1193), HSHCGI_PEA_{—}3_T7 (SEQ ID NO:1194), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T11 (SEQ ID NO:1198), HSHCGI_PEA_{—}3_T12 (SEQ ID NO:1199), HSHCGI_PEA_{—}3_T13 (SEQ ID NO:1200), HSHCGI_PEA_{—}3_T18 (SEQ ID NO:1204) and HSHCGI_PEA_{—}3_T23 (SEQ ID NO:1209). Table 64 below describes the starting and ending position of this segment on each transcript.

TABLE 64
Segment location on transcripts

| | Segment | Segment |
|---|---|---|
| | starting | ending |
| Transcript name | position | position |
| HSHCGI_PEA_3_T0 (SEQ ID NO: 1187) | 528 | 623 |
| HSHCGI_PEA_3_T1 (SEQ ID NO: 1188) | 556 | 651 |
| HSHCGI_PEA_3_T2 (SEQ ID NO: 1189) | 529 | 624 |
| HSHCGI_PEA_3_T3 (SEQ ID NO: 1190) | 556 | 651 |
| HSHCGI_PEA_3_T4 (SEQ ID NO: 1191) | 420 | 515 |
| HSHCGI_PEA_3_T5 (SEQ ID NO: 1192) | 556 | 651 |
| HSHCGI_PEA_3_T6 (SEQ ID NO: 1193) | 110 | 205 |
| HSHCGI_PEA_3_T7 (SEQ ID NO: 1194) | 556 | 651 |
| HSHCGI_PEA_3_T8 (SEQ ID NO: 1195) | 556 | 651 |
| HSHCGI_PEA_3_T9 (SEQ ID NO: 1196) | 556 | 651 |
| HSHCGI_PEA_3_T10 (SEQ ID NO: 1197) | 556 | 651 |
| HSHCGI_PEA_3_T11 (SEQ ID NO: 1198) | 528 | 623 |
| HSHCGI_PEA_3_T12 (SEQ ID NO: 1199) | 556 | 651 |
| HSHCGI_PEA_3_T13 (SEQ ID NO: 1200) | 528 | 623 |
| HSHCGI_PEA_3_T18 (SEQ ID NO: 1204) | 556 | 651 |
| HSHCGI_PEA_3_T23 (SEQ ID NO: 1209) | 556 | 651 |

Segment cluster HSHCGI_PEA_{—}3_node_{—}19 (SEQ ID NO:1232) according to the present invention can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T3 (SEQ ID NO:1190), HSHCGI_PEA_{—}3_T4 (SEQ ID NO:1191), HSHCGI_PEA_{—}3_T5 (SEQ ID NO:1192), HSHCGI_PEA_{—}3_T6 (SEQ ID NO:1193), HSHCGI_PEA_{—}3_T7 (SEQ ID NO:1194), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T12 (SEQ ID NO:1199), HSHCGI_PEA_{—}3_T14 (SEQ ID NO:1201) and HSHCGI_PEA_{—}3_T18 (SEQ ID NO:1204). Table 65 below describes the starting and ending position of this segment on each transcript.

TABLE 65
Segment location on transcripts

| | Segment | Segment |
|---|---|---|
| | starting | ending |
| Transcript name | position | position |
| HSHCGI_PEA_3_T0 (SEQ ID NO: 1187) | 855 | 877 |
| HSHCGI_PEA_3_T1 (SEQ ID NO: 1188) | 883 | 905 |
| HSHCGI_PEA_3_T2 (SEQ ID NO: 1189) | 856 | 878 |
| HSHCGI_PEA_3_T3 (SEQ ID NO: 1190) | 883 | 905 |
| HSHCGI_PEA_3_T4 (SEQ ID NO: 1191) | 747 | 769 |
| HSHCGI_PEA_3_T5 (SEQ ID NO: 1192) | 883 | 905 |
| HSHCGI_PEA_3_T6 (SEQ ID NO: 1193) | 437 | 459 |
| HSHCGI_PEA_3_T7 (SEQ ID NO: 1194) | 883 | 905 |
| HSHCGI_PEA_3_T8 (SEQ ID NO: 1195) | 883 | 905 |
| HSHCGI_PEA_3_T9 (SEQ ID NO: 1196) | 883 | 905 |
| HSHCGI_PEA_3_T10 (SEQ ID NO: 1197) | 883 | 905 |
| HSHCGI_PEA_3_T12 (SEQ ID NO: 1199) | 883 | 905 |
| HSHCGI_PEA_3_T14 (SEQ ID NO: 1201) | 753 | 775 |
| HSHCGI_PEA_3_T18 (SEQ ID NO: 1204) | 883 | 905 |

Segment cluster HSHCGI_PEA_{—}3_node_{—}21 (SEQ ID NO:1233) according to the present invention can be found in the following transcript(s): HSHCGI_PEA_{—}3_T13 (SEQ ID NO:1200). Table 66 below describes the starting and ending position of this segment on each transcript.

TABLE 66
Segment location on transcripts

| | Segment | Segment |
|---|---|---|
| | starting | ending |
| Transcript name | position | position |
| HSHCGI_PEA_3_T13 (SEQ ID NO: 1200) | 855 | 878 |

Segment cluster HSHCGI_PEA_{—}3_node_{—}22 (SEQ ID NO:1234) according to the present invention is supported by 6 libraries. The number of libraries was determined as previously described. This segment can be found in the following transcript(s): HSHCGI_PEA_{—}3_T5 (SEQ ID NO:1192), HSHCGI_PEA_{—}3_T8 (SEQ ID NO:1195), HSHCGI_PEA_{—}3_T12 (SEQ ID NO:1199) and HSHCGI_PEA_{—}3_T14 (SEQ ID NO:1201). Table 67 below describes the starting and ending position of this segment on each transcript.

TABLE 67
Segment location on transcripts

| | Segment | Segment |
|---|---|---|
| | starting | ending |
| Transcript name | position | position |
| HSHCGI_PEA_3_T5 (SEQ ID NO: 1192) | 1697 | 1758 |
| HSHCGI_PEA_3_T8 (SEQ ID NO: 1195) | 906 | 967 |
| HSHCGI_PEA_3_T12 (SEQ ID NO: 1199) | 1697 | 1758 |
| HSHCGI_PEA_3_T14 (SEQ ID NO: 1201) | 1567 | 1628 |

Segment cluster HSHCGI_PEA_{—}3_node_{—}23 (SEQ ID NO:1235) according to the present invention is supported by 30 libraries. The number of libraries was determined as previously described. This segment can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T3 (SEQ ID NO:1190), HSHCGI_PEA_{—}3_T4 (SEQ ID NO:1191), HSHCGI_PEA_{—}3_T5 (SEQ ID NO:1192), HSHCGI_PEA_{—}3_T6 (SEQ ID NO:1193), HSHCGI_PEA_{—}3_T8 (SEQ ID NO:1195), HSHCGI_PEA_{—}3_T9 (SEQ ID NO:1196), HSHCGI_PEA_{—}3_T12 (SEQ ID NO:1199), HSHCGI_PEA_{—}3_T13 (SEQ ID NO:1200), HSHCGI_PEA_{—}3_T14 (SEQ ID NO:1201) and HSHCGI_PEA_{—}3_T18 (SEQ ID NO:1204). Table 68 below describes the starting and ending position of this segment on each transcript.

TABLE 68
Segment location on transcripts

| | Segment | Segment |
|---|---|---|
| | starting | ending |
| Transcript name | position | position |
| HSHCGI_PEA_3_T0 (SEQ ID NO: 1187) | 878 | 921 |
| HSHCGI_PEA_3_T1 (SEQ ID NO: 1188) | 906 | 949 |
| HSHCGI_PEA_3_T2 (SEQ ID NO: 1189) | 879 | 922 |
| HSHCGI_PEA_3_T3 (SEQ ID NO: 1190) | 906 | 949 |
| HSHCGI_PEA_3_T4 (SEQ ID NO: 1191) | 770 | 813 |
| HSHCGI_PEA_3_T5 (SEQ ID NO: 1192) | 1759 | 1802 |
| HSHCGI_PEA_3_T6 (SEQ ID NO: 1193) | 460 | 503 |
| HSHCGI_PEA_3_T8 (SEQ ID NO: 1195) | 968 | 1011 |
| HSHCGI_PEA_3_T9 (SEQ ID NO: 1196) | 906 | 949 |
| HSHCGI_PEA_3_T12 (SEQ ID NO: 1199) | 1759 | 1802 |
| HSHCGI_PEA_3_T13 (SEQ ID NO: 1200) | 879 | 922 |
| HSHCGI_PEA_3_T14 (SEQ ID NO: 1201) | 1629 | 1672 |
| HSHCGI_PEA_3_T18 (SEQ ID NO: 1204) | 906 | 949 |

Segment cluster HSHCGI_PEA_{—}3_node_{—}24 (SEQ ID NO:1236) according to the present invention is supported by 38 libraries. The number of libraries was determined as previously described. This segment can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T3 (SEQ ID NO:1190), HSHCGI_PEA_{—}3_T4 (SEQ ID NO:1191), HSHCGI_PEA_{—}3_T5 (SEQ ID NO:1192), HSHCGI_PEA_{—}3_T6 (SEQ ID NO:1193), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T11 (SEQ ID NO:1198), HSHCGI_PEA_{—}3_T12 (SEQ ID NO:1199), HSHCGI_PEA_{—}3_T13 (SEQ ID NO:1200), HSHCGI_PEA_{—}3_T14 (SEQ ID NO:1201) and HSHCGI_PEA_{—}3_T18 (SEQ ID NO:1204). Table 69 below describes the starting and ending position of this segment on each transcript.

TABLE 69
Segment location on transcripts

| | Segment | Segment |
|---|---|---|
| | starting | ending |
| Transcript name | position | position |
| HSHCGI_PEA_3_T0 (SEQ ID NO: 1187) | 922 | 993 |
| HSHCGI_PEA_3_T1 (SEQ ID NO: 1188) | 950 | 1021 |
| HSHCGI_PEA_3_T2 (SEQ ID NO: 1189) | 923 | 994 |
| HSHCGI_PEA_3_T3 (SEQ ID NO: 1190) | 950 | 1021 |
| HSHCGI_PEA_3_T4 (SEQ ID NO: 1191) | 814 | 885 |
| HSHCGI_PEA_3_T5 (SEQ ID NO: 1192) | 1803 | 1874 |
| HSHCGI_PEA_3_T6 (SEQ ID NO: 1193) | 504 | 575 |
| HSHCGI_PEA_3_T8 (SEQ ID NO: 1195) | 1012 | 1083 |
| HSHCGI_PEA_3_T9 (SEQ ID NO: 1196) | 950 | 1021 |
| HSHCGI_PEA_3_T10 (SEQ ID NO: 1197) | 906 | 977 |
| HSHCGI_PEA_3_T11 (SEQ ID NO: 1198) | 855 | 926 |
| HSHCGI_PEA_3_T12 (SEQ ID NO: 1199) | 1803 | 1874 |
| HSHCGI_PEA_3_T13 (SEQ ID NO: 1200) | 923 | 994 |
| HSHCGI_PEA_3_T14 (SEQ ID NO: 1201) | 1673 | 1744 |
| HSHCGI_PEA_3_T18 (SEQ ID NO: 1204) | 950 | 1021 |

Segment cluster HSHCGI_PEA_{—}3_node_{—}27 (SEQ ID NO:1237) according to the present invention is supported by 43 libraries. The number of libraries was determined as previously described. This segment can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T3 (SEQ ID NO:1190), HSHCGI_PEA_{—}3_T4 (SEQ ID NO:1191), HSHCGI_PEA_{—}3_T5 (SEQ ID NO:1192), HSHCGI_PEA_{—}3_T6 (SEQ ID NO:1193), HSHCGI_PEA_{—}3_T7 (SEQ ID NO:1194), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T11 (SEQ ID NO:1198), HSHCGI_PEA_{—}3_T12 (SEQ ID NO:1199), HSHCGI_PEA_{—}3_T13 (SEQ ID NO:1200), HSHCGI_PEA_{—}3_T14 (SEQ ID NO:1201), HSHCGI_PEA_{—}3_T15 (SEQ ID NO:1202), HSHCGI_PEA_{—}3_T18 (SEQ ID NO:1204), HSHCGI_PEA_{—}3_T19 (SEQ ID NO:1205), HSHCGI_PEA_{—}3_T20 (SEQ ID NO:1206) and HSHCGI_PEA_{—}3_T24 (SEQ ID NO:1210). Table 70 below describes the starting and ending position of this segment on each transcript.

TABLE 70
Segment location on transcripts

| | Segment | Segment |
|---|---|---|
| | starting | ending |
| Transcript name | position | position |
| HSHCGI_PEA_3_T0 (SEQ ID NO: 1187) | 994 | 1068 |
| HSHCGI_PEA_3_T1 (SEQ ID NO: 1188) | 1022 | 1096 |
| HSHCGI_PEA_3_T2 (SEQ ID NO: 1189) | 995 | 1069 |
| HSHCGI_PEA_3_T3 (SEQ ID NO: 1190) | 1022 | 1096 |
| HSHCGI_PEA_3_T4 (SEQ ID NO: 1191) | 886 | 960 |
| HSHCGI_PEA_3_T5 (SEQ ID NO: 1192) | 1875 | 1949 |
| HSHCGI_PEA_3_T6 (SEQ ID NO: 1193) | 576 | 650 |
| HSHCGI_PEA_3_T7 (SEQ ID NO: 1194) | 906 | 980 |
| HSHCGI_PEA_3_T8 (SEQ ID NO: 1195) | 1084 | 1158 |
| HSHCGI_PEA_3_T9 (SEQ ID NO: 1196) | 1022 | 1096 |
| HSHCGI_PEA_3_T10 (SEQ ID NO: 1197) | 978 | 1052 |
| HSHCGI_PEA_3_T11 (SEQ ID NO: 1198) | 927 | 1001 |
| HSHCGI_PEA_3_T12 (SEQ ID NO: 1199) | 1875 | 1949 |
| HSHCGI_PEA_3_T13 (SEQ ID NO: 1200) | 995 | 1069 |
| HSHCGI_PEA_3_T14 (SEQ ID NO: 1201) | 1745 | 1819 |
| HSHCGI_PEA_3_T15 (SEQ ID NO: 1202) | 421 | 495 |
| HSHCGI_PEA_3_T18 (SEQ ID NO: 1204) | 1022 | 1096 |
| HSHCGI_PEA_3_T19 (SEQ ID NO: 1205) | 421 | 495 |
| HSHCGI_PEA_3_T20 (SEQ ID NO: 1206) | 421 | 495 |
| HSHCGI_PEA_3_T24 (SEQ ID NO: 1210) | 421 | 495 |

Segment cluster HSHCGI_PEA_{—}3_node_{—}31 (SEQ ID NO:1238) according to the present invention is supported by 34 libraries. The number of libraries was determined as previously described. This segment can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T3 (SEQ ID NO:1190), HSHCGI_PEA_{—}3_T4 (SEQ ID NO:1191), HSHCGI_PEA_{—}3_T5 (SEQ ID NO:1192), HSHCGI_PEA_{—}3_T6 (SEQ ID NO:1193), HSHCGI_PEA_{—}3_T7 (SEQ ID NO:1194), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T11 (SEQ ID NO:1198), HSHCGI_PEA_{—}3_T12 (SEQ ID NO:1199), HSHCGI_PEA_{—}3_T13 (SEQ ID NO:1200), HSHCGI_PEA_{—}3_T14 (SEQ ID NO:1201), HSHCGI_PEA_{—}3_T15 (SEQ ID NO:1202), HSHCGI_PEA_{—}3_T17 (SEQ ID NO:1203), HSHCGI_PEA_{—}3_T19 (SEQ ID NO:1205) and HSHCGI_PEA_{—}3_T20 (SEQ ID NO:1206). Table 71 below describes the starting and ending position of this segment on each transcript.

TABLE 71
Segment location on transcripts

| | Segment | Segment |
|---|---|---|
| | starting | ending |
| Transcript name | position | position |
| HSHCGI_PEA_3_T0 (SEQ ID NO: 1187) | 1069 | 1134 |
| HSHCGI_PEA_3_T1 (SEQ ID NO: 1188) | 1097 | 1162 |
| HSHCGI_PEA_3_T2 (SEQ ID NO: 1189) | 1070 | 1135 |
| HSHCGI_PEA_3_T3 (SEQ ID NO: 1190) | 1097 | 1162 |
| HSHCGI_PEA_3_T4 (SEQ ID NO: 1191) | 961 | 1026 |
| HSHCGI_PEA_3_T5 (SEQ ID NO: 1192) | 1950 | 2015 |
| HSHCGI_PEA_3_T6 (SEQ ID NO: 1193) | 651 | 716 |
| HSHCGI_PEA_3_T7 (SEQ ID NO: 1194) | 981 | 1046 |
| HSHCGI_PEA_3_T8 (SEQ ID NO: 1195) | 1159 | 1224 |
| HSHCGI_PEA_3_T9 (SEQ ID NO: 1196) | 1097 | 1162 |
| HSHCGI_PEA_3_T10 (SEQ ID NO: 1197) | 1053 | 1118 |
| HSHCGI_PEA_3_T11 (SEQ ID NO: 1198) | 1002 | 1067 |
| HSHCGI_PEA_3_T12 (SEQ ID NO: 1199) | 1950 | 2015 |
| HSHCGI_PEA_3_T13 (SEQ ID NO: 1200) | 1070 | 1135 |
| HSHCGI_PEA_3_T14 (SEQ ID NO: 1201) | 1820 | 1885 |
| HSHCGI_PEA_3_T15 (SEQ ID NO: 1202) | 496 | 561 |
| HSHCGI_PEA_3_T17 (SEQ ID NO: 1203) | 422 | 487 |
| HSHCGI_PEA_3_T19 (SEQ ID NO: 1205) | 496 | 561 |
| HSHCGI_PEA_3_T20 (SEQ ID NO: 1206) | 496 | 561 |

Segment cluster HSHCGI_PEA_{—}3_node_{—}35 (SEQ ID NO:1239) according to the present invention is supported by 38 libraries. The number of libraries was determined as previously described. This segment can be found in the following transcript(s): HSHCGI_PEA_{—}3_T0 (SEQ ID NO:1187), HSHCGI_PEA_{—}3_T1 (SEQ ID NO:1188), HSHCGI_PEA_{—}3_T2 (SEQ ID NO:1189), HSHCGI_PEA_{—}3_T3 (SEQ ID NO:1190), HSHCGI_PEA_{—}3_T4 (SEQ ID NO:1191), HSHCGI_PEA_{—}3_T5 (SEQ ID NO:1192), HSHCGI_PEA_{—}3_T6 (SEQ ID NO:1193), HSHCGI_PEA_{—}3_T7 (SEQ ID NO:1194), HSHCGI_PEA_{—}3_T8 (SEQ ID NO:1195), HSHCGI_PEA_{—}3_T9 (SEQ ID NO:1196), HSHCGI_PEA_{—}3_T10 (SEQ ID NO:1197), HSHCGI_PEA_{—}3_T11 (SEQ ID NO:1198), HSHCGI_PEA_{—}3_T12 (SEQ ID NO:1199), HSHCGI_PEA_{—}3_T13 (SEQ ID NO:1200), HSHCGI_PEA_{—}3_T14 (SEQ ID NO:1201), HSHCGI_PEA_{—}3_T15 (SEQ ID NO:1202), HSHCGI_PEA_{—}3_T17 (SEQ ID NO:1203), HSHCGI_PEA_{—}3_T19 (SEQ ID NO:1205) and HSHCGI_PEA_{—}3_T20 (SEQ ID NO:1206). Table 72 below describes the starting and ending position of this segment on each transcript.

TABLE 72
Segment location on transcripts

| | Segment | Segment |
|---|---|---|
| | starting | ending |
| Transcript name | position | position |
| HSHCGI_PEA_3_T0 (SEQ ID NO: 1187) | 1679 | 1791 |
| HSHCGI_PEA_3_T1 (SEQ ID NO: 1188) | 1707 | 1819 |
| HSHCGI_PEA_3_T2 (SEQ ID NO: 1189) | 1680 | 1792 |
| HSHCGI_PEA_3_T3 (SEQ ID NO: 1190) | 1481 | 1593 |
| HSHCGI_PEA_3_T4 (SEQ ID NO: 1191) | 1571 | 1683 |
| HSHCGI_PEA_3_T5 (SEQ ID NO: 1192) | 2560 | 2672 |
| HSHCGI_PEA_3_T6 (SEQ ID NO: 1193) | 1261 | 1373 |
| HSHCGI_PEA_3_T7 (SEQ ID NO: 1194) | 1591 | 1703 |
| HSHCGI_PEA_3_T8 (SEQ ID NO: 1195) | 1769 | 1881 |
| HSHCGI_PEA_3_T9 (SEQ ID NO: 1196) | 2019 | 2131 |
| HSHCGI_PEA_3_T10 (SEQ ID NO: 1197) | 1663 | 1775 |
| HSHCGI_PEA_3_T11 (SEQ ID NO: 1198) | 1612 | 1724 |
| HSHCGI_PEA_3_T12 (SEQ ID NO: 1199) | 2872 | 2984 |
| HSHCGI_PEA_3_T13 (SEQ ID NO: 1200) | 1680 | 1792 |
| HSHCGI_PEA_3_T14 (SEQ ID NO: 1201) | 2430 | 2542 |
| HSHCGI_PEA_3_T15 (SEQ ID NO: 1202) | 1106 | 1218 |
| HSHCGI_PEA_3_T17 (SEQ ID NO: 1203) | 1344 | 1456 |
| HSHCGI_PEA_3_T19 (SEQ ID NO: 1205) | 1418 | 1530 |
| HSHCGI_PEA_3_T20 (SEQ ID NO: 1206) | 880 | 992 |

### Variant Protein Alignment to the Previously Known Protein:

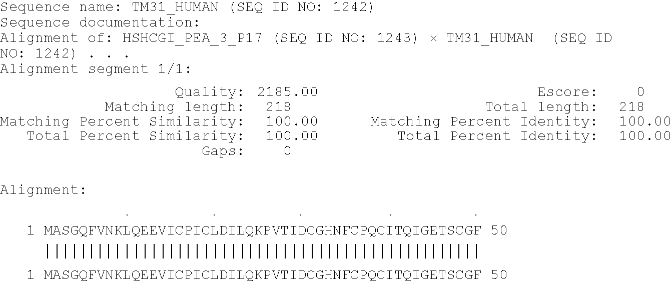

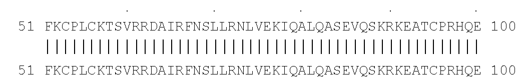

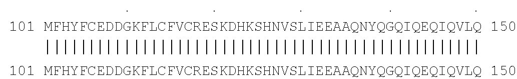

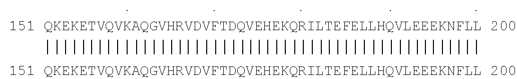

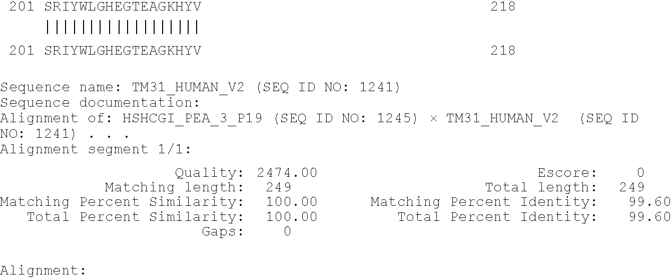

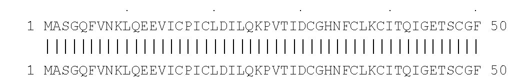

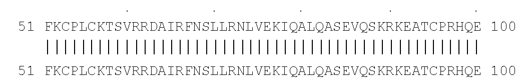

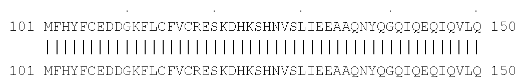

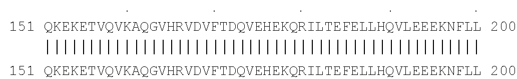

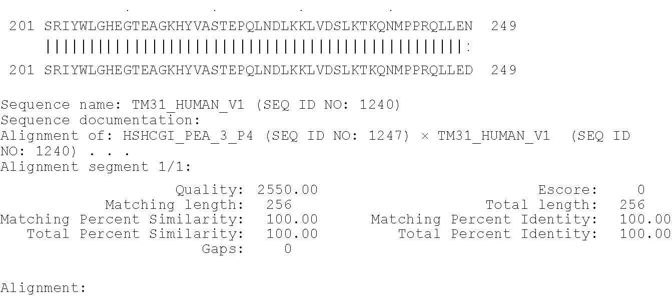

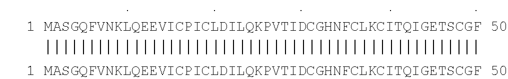

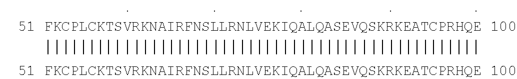

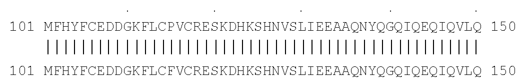

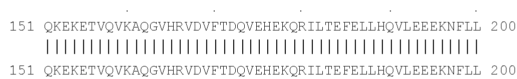

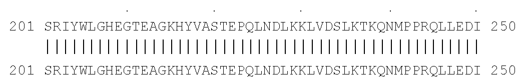

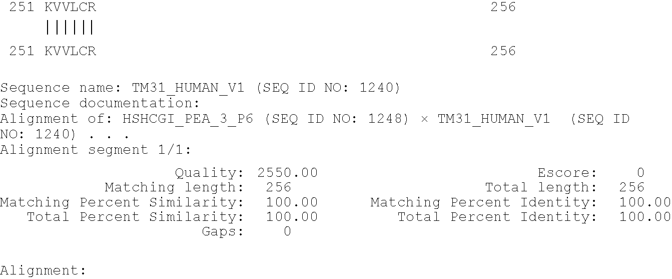

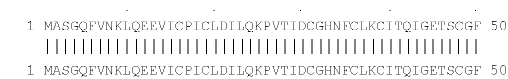

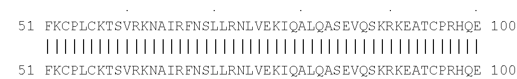

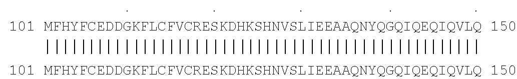

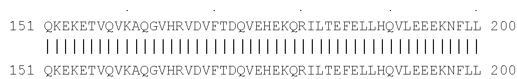

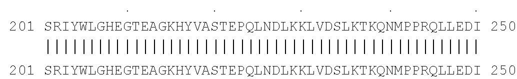

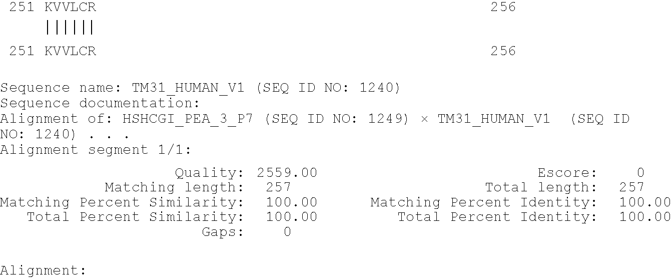

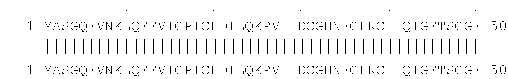

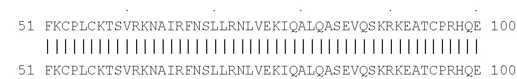

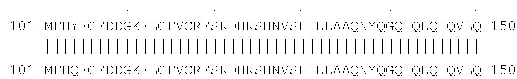

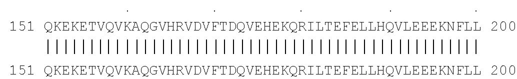

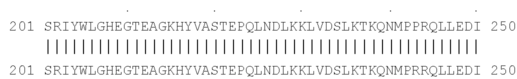

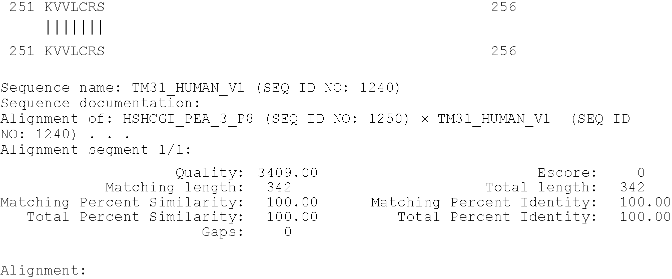

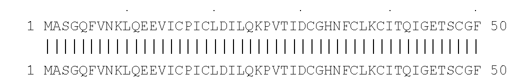

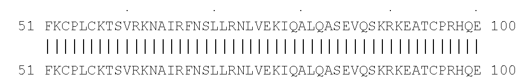

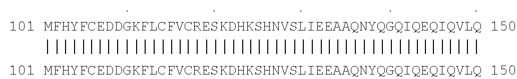

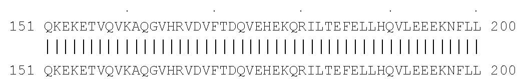

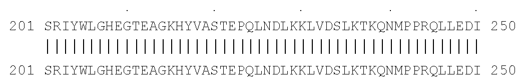

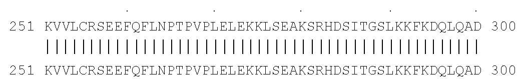

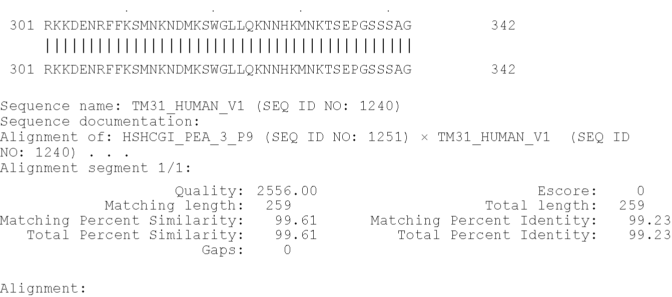

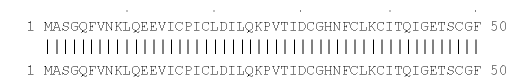

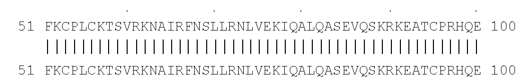

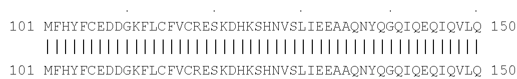

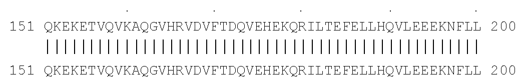

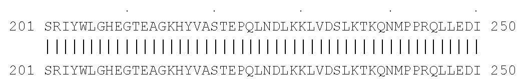

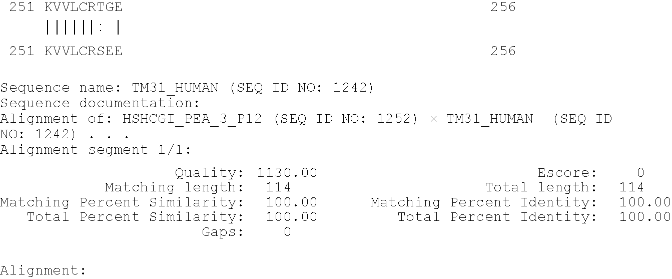

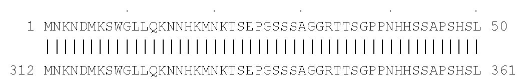

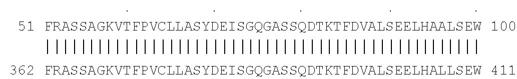

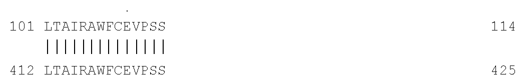

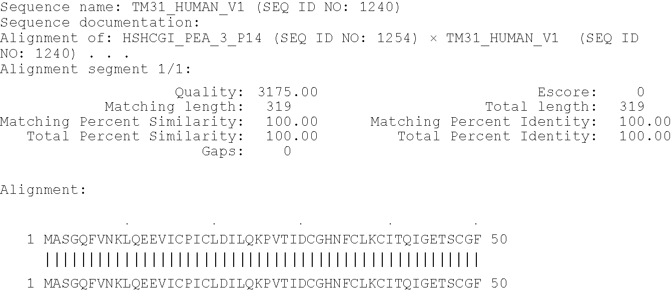

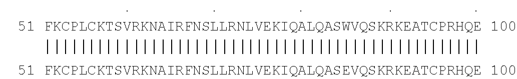

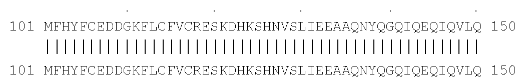

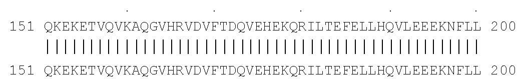

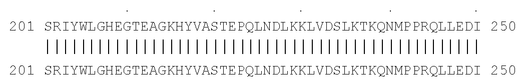

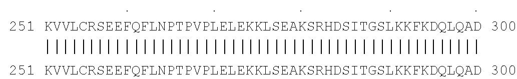

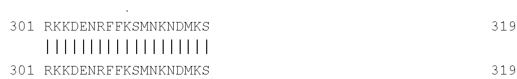

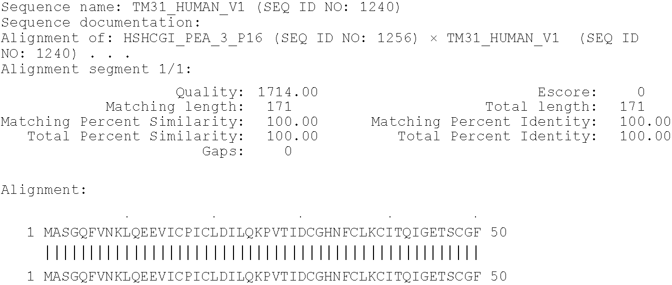

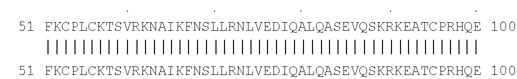

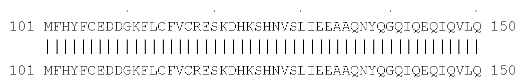

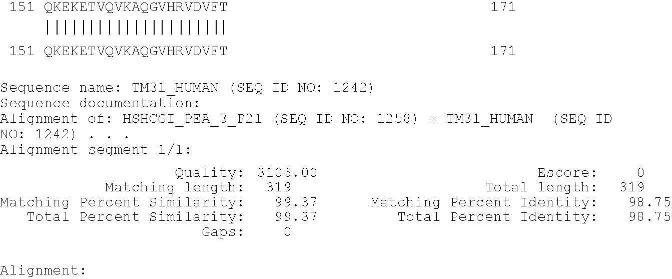

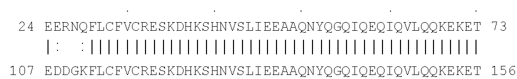

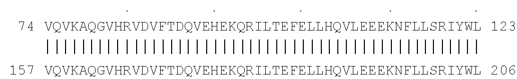

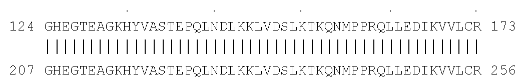

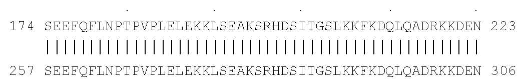

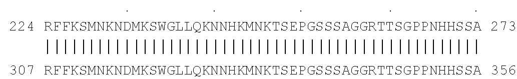

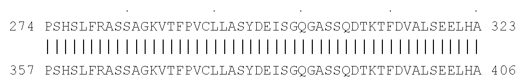

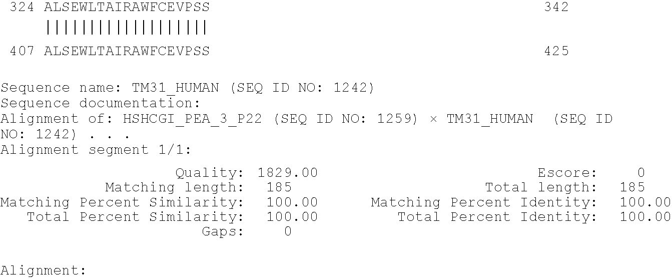

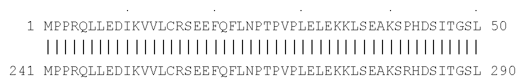

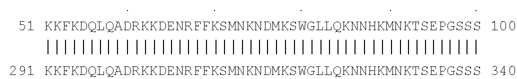

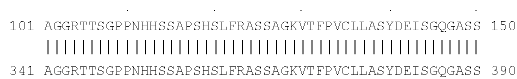

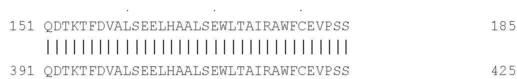

### Expression of TRIM31 Tripartite Motif HSHCGI Transcripts which are Detectable by Amplicon as Depicted in Sequence Name HSHCGI Seg20 (SEQ ID NO:1378) in Normal and Cancerous Colon Tissues

Expression of TRIM31 tripartite motif transcripts detectable by or according to seg20, HSHCGIseg20 amplicon (SEQ ID NO: 1378) and HSHCGIseg20F (SEQ ID NO: 1376) HSHCGIseg20R (SEQ ID NO: 1377) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing samples”), to obtain a value of fold up-regulation for each sample relative to median of the normal PM samples.

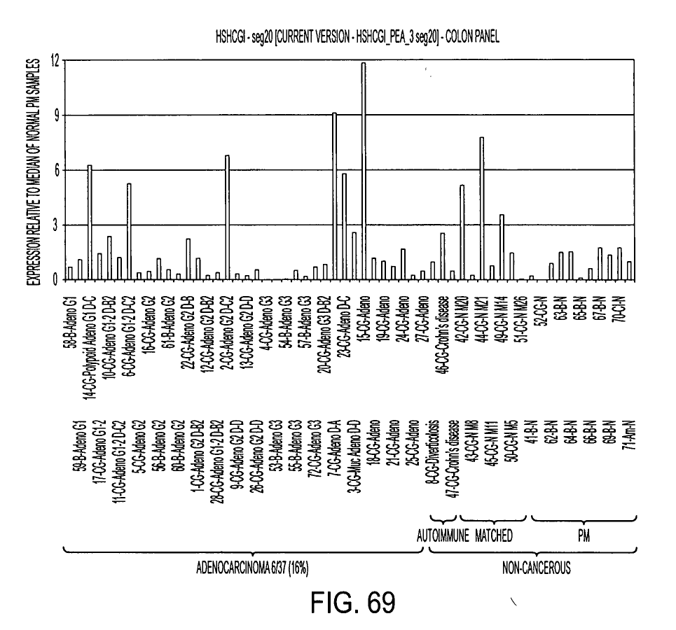

FIG. 69 is a histogram showing over expression of the above-indicated TRIM31 tripartite motif transcripts in cancerous colon samples relative to the normal samples. The number and percentage of samples that exhibit at least 3 fold over-expression, out of the total number of samples tested is indicated in the bottom.

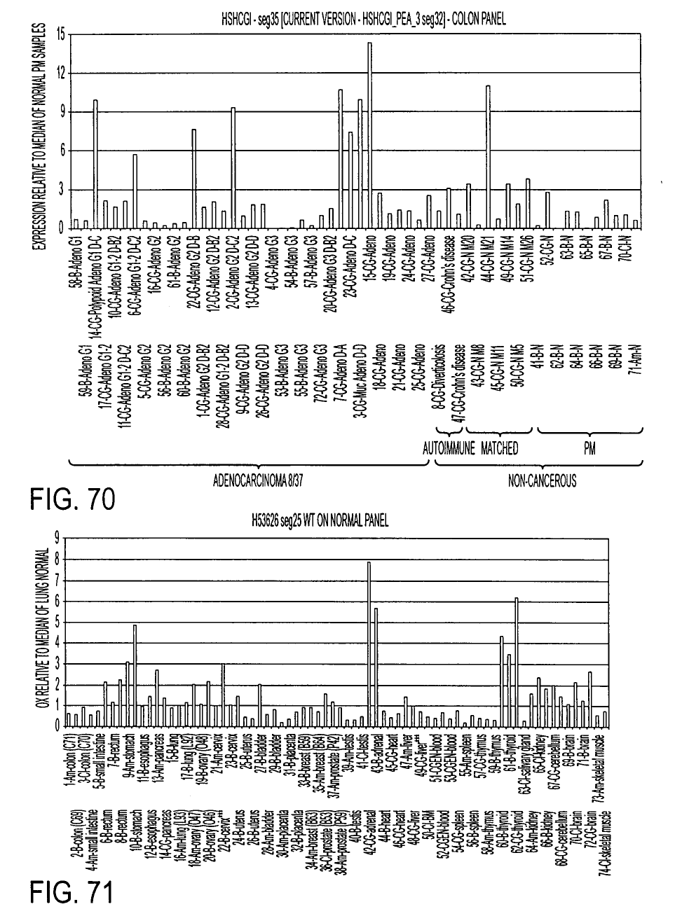

FIG. 70 is a histogram showing over expression of the above-indicated TRIM31 tripartite motif transcripts in cancerous colon samples relative to the normal samples. The number and percentage of samples that exhibit at least 3 fold over-expression, out of the total number of samples tested is indicated in the bottom.

As is evident from FIG. 70, the expression of TRIM31 tripartite motif transcripts detectable by the above amplicon in cancer samples was significantly higher than in the non-cancerous samples (Sample Nos. 41-45, 49-52, 62-67, 69-71 Table 1, , “Tissue samples in testing samples”). Notably an over-expression of at least 3 fold was found in 8 out of 37 adenocarcinoma samples,

Statistical analysis was applied to verify the significance of these results, as described below.

The P value for the difference in the expression levels of TRIM31 tripartite motif transcripts detectable by the above amplicon in colon cancer samples versus the normal tissue samples was determined by T test as 7.56E-03.

Primer pairs are also optionally and preferably encompassed within the present invention; for example, for the above experiment, the following primer pair was used as a non-limiting illustrative example only of a suitable primer pair: HSHCGIseg35F forward primer (SEQ ID NO: 1379); and HSHCGIseg35R reverse primer (SEQ ID NO: 1380).

The present invention also preferably encompasses any amplicon obtained through the use of any suitable primer pair; for example, for the above experiment, the following amplicon was obtained as a non-limiting illustrative example only of a suitable amplicon: HSHCGIseg35 (SEQ ID NO: 1381).

Forward primer (SEQ ID NO: 1379):
TAAGTCTACAGGTGGTCAAAATGCTG
Reverse primer (SEQ ID NO: 1380):
GGAGCGCCCTCTGTTTCC
Amplicon (SEQ ID NO: 1381):
TAAGTCTACAGGTGGTCAAAATGCTGTATCCACCCAATTCCACTAAATGG
AATAAATGAATAAATGAATGAATTCATTTATTCCATTTCCTCAGTTCCTC
CCCAAATTACACCTCTGCCAGGAAACAGAGGGCGCTCC

It should be noted that for R30650_PEA_{—}2-seg73, no differential expression was observed in one Q-PCR experiment carried out with colon panel. For HUMCEA_PEA_{—}1 seg 6—no differential expression was observed in one Q-PCR experiment carried out with colon panel.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

As is evident from FIG. 69, the expression of TRIM31 tripartite motif transcripts detectable by the above amplicon in cancer samples was higher than in the non-cancerous samples (Sample Nos. 41, 52, 62-67, 69-71, Table 1, “Tissue samples in testing samples”). Notably an over-expression of at least 3 fold was found in 6 out of 37 adenocarcinoma samples,

Statistical analysis was applied to verify the significance of these results, as described below.

The P value for the difference in the expression levels of TRIM31 tripartite motif transcripts detectable by the above amplicon in colon cancer samples versus the normal tissue samples was determined by T test as 6.58E-02.

Primer pairs are also optionally and preferably encompassed within the present invention; for example, for the above experiment, the following primer pair was used as a non-limiting illustrative example only of a suitable primer pair: HSHCGIseg20F forward primer (SEQ ID NO: 1376); and HSHCGIseg20R reverse primer (SEQ ID NO: 1377).

The present invention also preferably encompasses any amplicon obtained through the use of any suitable primer pair; for example, for the above experiment, the following amplicon was obtained as a non-limiting illustrative example only of a suitable amplicon: HSHCGIseg20 (SEQ ID NO: 1378).

Forward primer (SEQ ID NO: 1376):
TGCCTACTGATTCATCCACATACA
Reverse primer (SEQ ID NO: 1377):
GCATTCCCCGGCTGC
Amplicon (SEQ ID NO: 1378):
TGCCTACTGATTCATCCACATACAATTCTCAGCGTATATCCAAATGCAGT
CAACATTCCTCTCTCAGAAATACCCACCCACCTCTAACTCTGCATTCATA
CATTTAGGCTGCAGCCGGGGAATGC

### Expression of TRIM31 Tripartite Motif HSHCGI Transcripts which are Detectable by Amplicon as Depicted in Sequence Name HSHCGI Seg35 (SEQ ID NO: 1381) in Normal and Cancerous Colon Tissues

Expression of TRIM31 tripartite motif transcripts detectable by or according to seg35, HSHCGIseg35 amplicon (SEQ ID NO: 1381) and HSHCGIseg35F (SEQ ID NO: 1379) HSHCGIseg35R (SEQ ID NO: 1380) primers was measured by real time PCR. In parallel the expression of four housekeeping genes —PBGD (GenBank Accession No. BC019323 (SEQ ID NO:1576); amplicon—PBGD-amplicon, SEQ ID NO:531), HPRT1 (GenBank Accession No. NM_{—}000194 (SEQ ID NO:1577); amplicon—HPRT1-amplicon, SEQ ID NO:612), G6PD (GenBank Accession No. NM_{—}000402 (SEQ ID NO:1578); G6PD amplicon, SEQ ID NO:615), RPS27A (GenBank Accession No. NM_{—}002954 (SEQ ID NO:1579); RPS27A amplicon, SEQ ID NO:1261), was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (Sample Nos. 41, 52, 62-67, 69-71, Table 1, above, “Tissue samples in testing samples”), to obtain a value of fold up-regulation for each sample relative to median of the normal PM samples.

What is claimed is:

1. An isolated polynucleotide comprising the polynucleotide sequence set forth in a member selected from the group consisting of SEQ ID NOs: 1-522, 643-678, 693-702, 705-789, 806-862, 871-921, 929-985, 998-1061, 1081-1140, 1157-1182, 1187-1239, 1276, 1279, 1282, 1285, 1288, 1291, 1294, 1297, 1300, 1303, 1306, 1309, 1312, 1315, 1318, 1321, 1331-1334, 1337-1339, 1342, 1345, 1348, 1351, 1354, 1357, 1360, 1363, 1366, 1369, 1372, 1375, 1378, 1381, 1398-1441, 1586, 1589 and 1592, or a sequence at least about 95% identical thereto.

2. An isolated polypeptide comprising the polypeptide sequence set forth in a member selected from the group consisting of SEQ ID NOs: 524-528, 534-611, 683-692, 703-704, 792-805, 864-870, 923-928, 991-997, 1063-1080, 1151-1156, 1183-1186, 1243-1259, and 1442-1575.

3. An expression vector comprising the polynucleotide sequence according to claim 1.

4. A host cell comprising the vector of claim 3.

5. A process for producing a polypeptide comprising:
culturing the host cell according to claim 4 under conditions suitable to produce the polypeptide encoded by said polynucleotide; and recovering said polypeptide.

6. An isolated primer pair, comprising the pair of nucleic acid sequences selected from the group consisting of: SEQ NOs: 1274-1275, 1277-1278, 1280-1281, 1283-1284, 1286-1287, 1289-1290, 1292-1293, 1298-1299, 1301-1302, 1304-1305, 1307-1308, 1310-1311, 1313-1314, 1316-1317, 1319-1320, 1335-1336, 1340-1341, 1343-1344, 1346-1347, 1349-1350, 1352-1353, 1355-1356, 1358-1359, 1361-1362, 1364-1365, 1367-1368, 1370-1371, 1373-1374, 1376-1377, 1379-1380, 1584-1585, 1587-1588, and 1590-1591.

7. An antibody to specifically bind to the polypeptide of claim 2.

8. A kit for detecting colon cancer, comprising at least one primer pair of claim 6.

9. A kit for detecting colon cancer, comprising the antibody of claim 7.

10. The kit of claim 9, wherein said immunoassay is selected from the group consisting of an enzyme linked immunosorbent assay (ELISA), an immunoprecipitation assay, an immunofluorescence analysis, an enzyme immunoassay (EIA), a radioimmunoassay (RIA), or a Western blot analysis.

11. A method for detecting colon cancer, comprising detecting overexpression of the polynucleotide sequence set forth in a member selected from the group consisting of SEQ ID NOs: 1-522, 643-678, 693-702, 705-789, 806-862, 871-921, 929-985, 998-1061, 1081-1140, 1157-1182, 1187-1239, 1276, 1279, 1282, 1285, 1288, 1291, 1294, 1297, 1300, 1303, 1306, 1309, 1312, 1315, 1318, 1321, 1331-1334, 1337-1339, 1342, 1345, 1348, 1351, 1354, 1357, 1360, 1363, 1366, 1369, 1372, 1375, 1378, 1381, 1398-1441, 1586, 1589 and 1592, or a sequence at least about 95% identical thereto in a sample from a patient.

12. The method of claim 11, wherein said detecting overexpression comprises performing nucleic acid amplification.

13. A method for detecting colon cancer, comprising detecting overexpression of the polypeptide comprising the polypeptide sequence set forth in a member selected from the group consisting of SEQ ID NOs: 524-528, 534-611, 683-692, 703-704, 792-805, 864-870, 923-928, 991-997, 1063-1080, 1151-1156, 1183-1186, 1243-1259, and 1442-1575 in a sample from a patient.

14. The method of claim 13, wherein said detecting comprises detecting binding of the antibody of claim 7 to the polypeptide comprising the polypeptide sequence set forth in a member selected from the group consisting of SEQ ID NOs: 524-528, 534-611, 683-692, 703-704, 792-805, 864-870, 923-928, 991-997, 1063-1080, 1151-1156, 1183-1186, 1243-1259, and 1442-1575 in a sample from a patient.

15. A biomarker for detecting colon cancer, comprising an amino acid sequence of claim 2, marked with a label.

16. A method to screen for or to diagnose colon cancer, comprising detecting the disease with the biomarker of claim 15.

17. A method for monitoring disease progression, treatment efficacy or relapse of colon cancer, comprising detecting the disease with the biomarker of claim 15.

18. A method of selecting a therapy for colon cancer, comprising detecting the disease with the biomarker of claim 15 and selecting a therapy according to said detection.

19. A biomarker for detecting colon cancer, comprising a nucleotide acid sequence set forth in a member selected from the group consisting of SEQ ID NOs: 1-522, 643-678, 693-702, 705-789, 806-862, 871-921, 929-985, 998-1061, 1081-1140, 1157-1182, 1187-1239, 1276, 1279, 1282, 1285, 1288, 1291, 1294, 1297, 1300, 1303, 1306, 1309, 1312, 1315, 1318, 1321, 1331-1334, 1337-1339, 1342, 1345, 1348, 1351, 1354, 1357, 1360, 1363, 1366, 1369, 1372, 1375, 1378, 1381, 1398-1441, 1586, 1589 and 1592, or a sequence at least about 95% identical thereto.

20. A method to screen for or to diagnose colon cancer, comprising detecting the disease with the biomarker of claim 19.

21. A method for monitoring disease progression, treatment efficacy or relapse of colon cancer, comprising detecting the disease with the biomarker of claim 19.

22. A method of selecting a therapy for colon cancer, comprising detecting the disease with the biomarker of claim 19 and selecting a therapy according to said detection.
